(19) United States
(12) Patent Application Publication
(10) Pub. No.: US 2026/0286329 A1
(43) Pub. Date: Sep. 24, 2026

(54) Compositions, Methods, and Systems for Genomic Editing

(71) Applicant: Intellia Therapeutics, Inc., Cambridge, MA (US)

(72) Inventors: Sabin Mulepati, Somerville, MA (US); Christian Dombrowski, Auburndale, MA (US); Jasmine Josephine Bonanno, Cambridge, MA (US); Kritika Hanamshet, Belmont, MA (US); Anita Ramachandran, Medford, MA (US); Darwin Yang, Cambridge, MA (US); Sung Hee Choi, Brookline, MA (US); Pei Wang, Belmont, MA (US); Weijun Chen, Shrewsbury, MA (US); Rubina Giare Parmar, Acton, MA (US)

(73) Assignee: Intellia Therapeutics, Inc., Cambridge, MA (US)

(21) Appl. No.: 19/670,372

(22) Filed: May 7, 2026

Related U.S. Application Data
(63) Continuation of application No. PCT/US2024/055256, filed on Nov. 8, 2024.
(60) Provisional application No. 63/598,064, filed on Nov. 10, 2023.
(60) Provisional application No. 63/660,308, filed on Jun. 14, 2024.
(60) Provisional application No. 63/694,651, filed on Sep. 13, 2024.
(60) Provisional application No. 63/716,220, filed on Nov. 4, 2024.

(51) Int. Cl. C12N 9/22; C12N 9/12; C12N 15/11; C12N 15/115; C12N 15/90

(52) U.S. Cl. CPC C12N 9/226; C12N 9/1252; C12N 15/11; C12N 15/115; C12N 15/907; C12Y 207/07007; C12N 2310/16; C12N 2310/20; C12N 2310/315; C12N 2310/321; C12N 2310/531

(57) ABSTRACT

The present disclosure relates to compositions, methods, and systems for genomic editing. In certain aspects, the compositions, methods, and systems provided herein can incorporate a SpyCas9 nickase, a DNA-dependent DNA polymerase, and/or a template guide RNA for template-based genome editing applications.

### CROSS-REFERENCE TO RELATED APPLICATIONS

This application is a continuation of International Application No. PCT/US2024/055256, filed Nov. 8, 2024, which claims the benefit of priority to U.S. Provisional Application No. 63/598,064, filed Nov. 10, 2023; U.S. Provisional Application No. 63/660,308, filed Jun. 14, 2024; U.S. Provisional Application No. 63/694,651, filed Sep. 13, 2024; and U.S. Provisional Application No. 63/716,220, filed Nov. 4, 2024, the content of each of which is herein incorporated by reference in its entirety.

### REFERENCE TO ELECTRONIC SEQUENCE LISTING

This application contains a sequence listing, which has been submitted electronically in XML file format and is hereby incorporated by reference in its entirety. Said XML file, created on Nov. 7, 2024, is named “01155-0062-00PCT.xml” and is 7,378,770 bytes in size.

### INTRODUCTION AND SUMMARY

The present disclosure relates to compositions, methods, and systems for genomic editing.

The ability to introduce precise and reproducible edits into the genome of a cell is of interest for gene editing and clinical therapeutic applications. The ability to generate a templated edit at a disease-associated locus-thereby replacing the endogenous, pathogenic sequence with a non-pathogenic, exogenous sequence—is therefore of great therapeutic interest.

CRISPR/Cas9 genome editing has been demonstrated to be highly efficient; however, it has been challenging to generate templated genomic edits. Conventional approaches for generating targeted genomic modifications often exploit the ability of the homology-directed repair (HDR) pathway to repair DNA breaks using a synthetic repair template comprising an insertion, deletion, or substitution. However, HDR is largely restricted to actively dividing cells, limiting its efficacy (Cox et al., Nature Medicine, 21:121-131 (2015). Thus, there is a need for a more robust and efficient approach for generating templated edits within the genome of a cell.

The methods provided herein comprise using a SpyCas9 nickase, DNA-dependent DNA polymerase, and template guide RNA (tgRNA) for template-based genome editing applications, providing substantial advantages over traditional methods.

In some aspects, the present disclosure provides a system comprising a DNA-dependent DNA polymerase (e.g., a T5 DNA polymerase or a PolK DNA polymerase), a SpyCas9 nickase (e.g., a SpyCas9 comprising a mutation in the HINH nuclease domain, e.g., a H840A substitution), and a template guide RNA (tgRNA) comprising a spacer, a scaffold, a template, and a DNA-dependent DNA polymerase recruiting sequence (DRS). In some embodiments, the system further comprises one or more accessory factors (e.g., nicking guide RNA (ngRNA), Fen1 or VPX). In some embodiments the system further comprises a chromatin remodeler (e.g., HMGB1). Such systems can be provided in a variety of configurations, as described herein. These components can be used in the compositions and methods described herein.

Accordingly, in some embodiments, the present disclosure provides for a system comprising a DNA-dependent DNA polymerase, a SpyCas9 nickase (nCas9), and a template guide RNA (tgRNA), wherein the tgRNA comprises a first polynucleotide and a second polynucleotide, wherein: (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; (ii) a DNA-dependent DNA polymerase recruiting sequence (DRS) having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid. In some embodiments, the DRS of the tgRNA comprises RNA nucleotides.

In some embodiments, the DRS of the tgRNA is 6-20 nucleotides in length, optionally 6-17 or 10-14 nucleotides in length. In some embodiments, the DRS of the tgRNA comprises DNA nucleotides. In some embodiments, the DRS of the tgRNA comprises both RNA nucleotide(s) and DNA nucleotide(s). In some embodiments, the DRS of the tgRNA comprises both DNA nucleotides and RNA nucleotides. In some embodiments, the DRS comprises at least one of: (1) a sequence not fully complementary to the DRS-complementary region of the non-target strand of the DNA duplex target nucleic acid; (2) a 1-5 nucleotide, optionally a 1-3 nucleotide 3′ terminal tail comprising a 3′ terminal modified uridine nucleotide; or (3) a 5′ terminal (D1) DNA nucleotide and a penultimate 5′ terminal (D2) RNA nucleotide wherein the 3′ end of the first polynucleotide is covalently linked to the 5′ end of the second polynucleotide by a phosphorothioate or phosphodiester linkage.

In some embodiments, the present disclosure provides for a system comprising a DNA-dependent DNA polymerase, a SpyCas9 nickase (nCas9), and a template guide RNA (tgRNA), wherein the tgRNA comprises a first polynucleotide and a second polynucleotide, wherein: (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; (ii) a DNA-dependent DNA polymerase recruiting sequence (DRS) having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid, wherein the DRS comprises DNA nucleotides; and wherein the DRS comprises at least one of: (1) a sequence not fully complementary to the DRS-complementary region of the non-target strand of the DNA duplex target nucleic acid; (2) a 1-5 nucleotide, optionally a 1-3 nucleotide 3′ terminal tail comprising a 3′ terminal modified uridine nucleotide; or (3) a 5′ terminal (D1) DNA nucleotide and a penultimate 5′ terminal (D2) RNA nucleotide wherein the 3′ end of the first polynucleotide is covalently linked to the 5′ end of the second polynucleotide by a phosphorothioate or phosphodiester linkage. In some embodiments, the DRS of the tgRNA is 6-20 nucleotides in length, optionally 6-17 or 10-14 nucleotides in length.

In some embodiments, the present disclosure provides for a system comprising a DNA-dependent DNA polymerase, a SpyCas9 nickase (nCas9), and a template guide RNA (tgRNA), wherein the tgRNA comprises a first polynucleotide and a second polynucleotide, wherein: (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; (ii) a DNA-dependent DNA polymerase recruiting sequence (DRS) that is 6-20 nucleotides in length, optionally 6-17 or 10-14 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid, wherein the DRS comprises RNA nucleotides; and wherein the DRS comprises at least one of: (1) a sequence not fully complementary to the DRS-complementary region of the non-target strand of the DNA duplex target nucleic acid; (2) a 1-5 nucleotide, optionally a 1-3 nucleotide 3′ terminal tail comprising a 3′ terminal modified uridine nucleotide; or (3) a 5′ terminal (D1) DNA nucleotide and a penultimate 5′ terminal (D2) RNA nucleotide wherein the 3′ end of the first polynucleotide is covalently linked to the 5′ end of the second polynucleotide by a phosphorothioate or phosphodiester linkage.

In some embodiments, the present disclosure provides for a system comprising a DNA polymerase kappa (polK), a SpyCas9 nickase (nCas9), and a template guide RNA (tgRNA), wherein the tgRNA comprises a first polynucleotide and a second polynucleotide, wherein (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; (ii) a DNA-dependent DNA polymerase recruiting sequence (DRS) that is about 6-18 nucleotides in length, optionally about 8-18 or 10-12 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid. In some embodiments, the DRS of the tgRNA comprises RNA nucleotides. In some embodiments, the DRS of the tgRNA comprises DNA nucleotides. In some embodiments, the DRS of the tgRNA comprises both RNA nucleotide(s) and DNA nucleotide(s).

In some embodiments, the present disclosure provides for a system comprising a DNA polymerase kappa (polK), a SpyCas9 nickase (nCas9), and a template guide RNA (tgRNA), wherein the tgRNA comprises a first polynucleotide and a second polynucleotide, wherein (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; (ii) a DNA-dependent DNA polymerase recruiting sequence (DRS) that is about 6-18 nucleotides in length, optionally about 8-18 or 10-12 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid, wherein the DRS comprises RNA nucleotides.

In some embodiments, the present disclosure provides for a system comprising a DNA polymerase kappa (polK), a SpyCas9 nickase (nCas9), and a template guide RNA (tgRNA), wherein the tgRNA comprises a first polynucleotide and a second polynucleotide, wherein (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; (ii) a DNA-dependent DNA polymerase recruiting sequence (DRS) that is about 6-18 nucleotides in length, optionally about 8-18 or 10-12 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid, wherein the DRS comprises DNA nucleotides.

In some embodiments, the present disclosure provides for a system comprising a T5 DNA polymerase (T5 pol), a SpyCas9 nickase (nCas9), and a template guide RNA (tgRNA), wherein the tgRNA comprises a first polynucleotide and a second polynucleotide, wherein (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; and (ii) a DNA-dependent DNA polymerase recruiting sequence (DRS) that is 6-20 nucleotides in length, optionally 6-17 or 10-14 nucleotides in length, and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid comprising nucleotides 5-10 5′ of the PAM in the non-target strand of the DNA duplex target nucleic acid. In some embodiments, the DRS of the tgRNA comprises DNA nucleotides. In some embodiments, the DRS of the tgRNA comprises both RNA nucleotide(s) and DNA nucleotide(s).

In some embodiments, the present disclosure provides for a system comprising a T5 DNA polymerase (T5 pol), a SpyCas9 nickase (nCas9), and a template guide RNA (tgRNA), wherein the tgRNA comprises a first polynucleotide and a second polynucleotide, wherein (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; and (ii) a DNA-dependent DNA polymerase recruiting sequence (DRS) that is 6-20 nucleotides in length, optionally 6-17 or 10-14 nucleotides in length, and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid comprising nucleotides 5-10 5′ of the PAM in the non-target strand of the DNA duplex target nucleic acid, wherein the DRS comprises RNA nucleotides.

In some embodiments, the present disclosure provides for a system comprising a T5 DNA polymerase (T5 pol), a SpyCas9 nickase (nCas9), and a template guide RNA (tgRNA), wherein the tgRNA comprises a first polynucleotide and a second polynucleotide, wherein (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; and (ii) a DNA-dependent DNA polymerase recruiting sequence (DRS) that is 6-20 nucleotides in length, optionally 6-17 or 10-14 nucleotides in length, and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid comprising nucleotides 5-10 5′ of the PAM in the non-target strand of the DNA duplex target nucleic acid, wherein the DRS comprises DNA nucleotides.

In some embodiments, the present disclosure provides for a fusion polypeptide comprising a polK comprising GE411-412RV mutations (i.e., mutations at G411R and E412V) relative to SEQ ID NO: 1021 (referred to herein as “polK (GE411-412RV)”), a SpyCas9 nickase (nCas9), and a heterologous nuclear localization signal (NLS), or a nucleic acid encoding the fusion polypeptide.

In some embodiments, the present disclosure provides for a fusion polypeptide comprising a T5 DNA polymerase comprising D164A and E166A mutations relative to SEQ ID NO: 1021, a SpyCas9 nickase (nCas9), and a heterologous nuclear localization signal (NLS), or a nucleic acid encoding the fusion polypeptide.

In some embodiments, the present disclosure provides for a fusion polypeptide comprising a T5 DNA polymerase comprising D164A and E166A mutations relative to SEQ ID NO: 1116, a SpyCas9 nickase (nCas9), and a heterologous nuclear localization signal (NLS), or a nucleic acid encoding the fusion polypeptide.

In some embodiments, the present disclosure provides for a guide RNA comprising the sequence of GUUUUAGA(L3)AAGUUAAAAUAAGGCUAGUCCGUUAUCAC(L1)GGGCACCGAGUCGGUGC U (SEQ ID NO: 370), wherein L1 denotes that the linker is S18, and L3 denotes that the linker is S6.

### FIGURE LEGENDS

FIG. 1 shows the steps of template-based genomic editing by SpyCas9 nickase, DNA-dependent DNA polymerase, and a template guide RNA, including subsequent repair of the nick site.

FIGS. 2A-2B show schematics of an exemplary template guide RNA complexed with a DNA duplex target nucleic acid indicated as being present in genomic DNA. The guide RNA spacer is complementary to the target strand of the DNA duplex target nucleic acid and the DNA polymerase recruiting sequence (DRS) is complementary to the non-target strand of the DNA duplex target nucleic acid at a site 5′ of the nick in the non-target strand of the DNA duplex target nucleic acid. The template sequence represents incorporation of two templated edits in the target nucleic acid.

FIGS. 3A-3D show exemplary systems for template-based genomic editing. FIG. 3A shows an exemplary complex comprising (1) a fusion protein comprising a SpyCas9 nickase and DNA-dependent DNA polymerase, (2) a template guide RNA including the DNA polymerase recruiting sequence (DRS) and the template sequence encoding an edit, and (3) the target strand of the DNA duplex target nucleic acid complementary to the guide RNA spacer and the non-target strand of the DNA duplex target nucleic acid complementary to the DRS. FIG. 3B shows an exemplary complex comprising (1) a fusion protein comprising a SpyCas9 nickase, DNA-dependent DNA polymerase, and aptamer-binding domain (ABD), (2) an sgRNA, (3) a separable 3′ extension comprising a DRS, template sequence encoding an edit, and an aptamer, and (4) the target strand of the DNA duplex target nucleic acid complementary to the guide RNA spacer and the non-target strand of the DNA duplex target nucleic acid complementary to the DRS. FIG. 3C shows an exemplary complex comprising (1) a SpyCas9 nickase, (2) a DNA-dependent DNA polymerase and ABD, (3) an sgRNA, (4) a separable 3′ extension comprising a DRS, template sequence encoding an edit, and an aptamer, and (5) the target strand of the DNA duplex target nucleic acid complementary to the guide RNA spacer and the non-target strand of the DNA duplex target nucleic acid complementary to the DRS.

FIG. 3D shows the steps of template-based genome editing in the presence of enhancers that (1) promote efficient DNA synthesis (indicated as “AF1”) and (2) mediate the removal of a flap 3′ of a nick (indicated as “AF2”).

FIG. 4 shows a schematic of an exemplary 3′ extension. The MS2 aptamer sequence with the exemplary chemical modifications is a generic structure, whereas the template and DRS sequences are directed to a particular target site to generate a particular edit. RNA nucleotides are indicated in black text, DNA nucleotides are indicated in gray text, 2′-O-Me modified nucleotides are shown in circles and phosphorothioate (PS) linkages are shown with “*”.

FIG. 5 shows a schematic of an exemplary template guide RNA. Dashed black lines indicate nucleotides that the upper stem portion may be shortened, while the arrow indicates nucleotides that may be replaced with an internal linker.

FIG. 6A shows a spacer and a scaffold of an exemplary guide RNA (which may or may not further comprise a 3′ extension) with a first internal linker represented by the line between nucleotides 28 and 41; and a second internal linker represented by the line between nucleotides 69 and 80 (SEQ ID NO: 371). Nucleotide numbering is based on the exemplary SpyCas9 Guide RNA as shown in Table 5B (SEQ ID NO: 115). 2′-O-Me modified nucleotides are shown in circles and phosphorothioate (PS) linkages are shown with “*”.

FIG. 6B shows a schematic of an exemplary template guide RNA (including the SpyCas9 guide RNA sequence of SEQ ID NO: 312). 2′-O-Me modified nucleotides are shown in circles and phosphorothioate (PS) linkages are shown with “*”.

FIG. 6C shows a schematic of an exemplary template guide RNA (including the SpyCas9 guide RNA sequence of SEQ ID NO: 434). 2′-O-Me modified nucleotides are shown in circles and phosphorothioate (PS) linkages are shown with “*”.

FIG. 6D shows a schematic of an exemplary template guide RNA (including the SpyCas9 guide RNA sequence of SEQ ID NO: 349). Black curved lines indicate an internal linker (S18 PEG linker) replacing nucleotides. 2′-O-Me modified nucleotides are shown in circles and phosphorothioate (PS) linkages are shown with “*”.

FIG. 6E shows a schematic of an exemplary template guide RNA (including the SpyCas9 guide RNA sequence of SEQ ID NO: 348). Black curved lines indicate an internal linker (S18 PEG linker) replacing nucleotides. 2′-O-Me modified nucleotides are shown in circles and phosphorothioate (PS) linkages are shown with “*”.

FIG. 6F shows a schematic of an exemplary template guide RNA (including the SpyCas9 guide RNA sequence of SEQ ID NO: 347). Black curved lines indicate an internal linker (S18 PEG linker) replacing nucleotides. 2′-O-Me modified nucleotides are shown in circles and phosphorothioate (PS) linkages are shown with “*”.

FIG. 6G shows a schematic of an exemplary template guide RNA (including the SpyCas9 guide RNA sequence of SEQ ID NO: 438). Black curved lines indicate an internal linker (S18 PEG linker) replacing nucleotides. 2′-O-Me modified nucleotides are shown in circles and phosphorothioate (PS) linkages are shown with “*”.

FIG. 6H shows a schematic of an exemplary template guide RNA (including the SpyCas9 guide RNA sequence of SEQ ID NO: 320). 2′-O-Me modified nucleotides are shown in circles, phosphorothioate (PS) linkages are shown with “*”, and 2′-F modified nucleotides are shown as “fN,” where “N” may be any nucleotide.

FIG. 6I shows a schematic of an exemplary template guide RNA (including the SpyCas9 guide RNA sequence of SEQ ID NO: 319). 2′-O-Me modified nucleotides are shown in circles, phosphorothioate (PS) linkages are shown with “*”, and 2′-F modified nucleotides are shown as “fN,” where “N” may be any nucleotide.

FIG. 7 shows an exemplary nicking guide RNA (SEQ ID NO: 310). 2′-O-Me modified nucleotides are shown in circles and phosphorothioate (PS) linkages are shown with “*”.

FIG. 8 shows the mean percent indels measured at the HEK3 locus for a guide RNA scaffold screen with SpyCas9 mRNA in Huh7 cells.

FIG. 9 shows editing at the VEGFA locus with sgRNA (G034491; SEQ ID NO: 482) and MS2-loop-linked 3′-extensions comprising various RNA/DNA compositions, in the absence of a nicking guide RNA (ngRNA).

FIG. 10 shows editing at the VEGFA locus with sgRNA (G034491) and MS2-loop-linked 3′-extensions comprising various RNA/DNA compositions in the presence of ngRNA (G034622; SEQ ID NO: 527)

FIG. 11 shows the mean percent indels at the HEK3 locus or at the VEGFA locus in Huh7 cells using SpyCas9 mRNA with a sgRNA (G033301 targeting HEK3 or G034491 targeting VEGFA) and MS2-loop-linked 3′-extensions encoding various edits.

FIGS. 12A and 12B show the mean percent editing at the HEK3 locus or at the VEGFA locus in Huh7 cells using template based editing with nCas9-T5Pol(D164A,E166A)-MCP (FIG. 12A) or MCP-PolK-nCas9 (FIG. 12B).

FIG. 13 shows the mean percent editing at the HEK3 locus or at the VEGFA locus as encoded by MS2-loop-linked 3′-extensions in Huh7 cells using MCP-PolK-nCas9 without an ngRNA.

FIG. 14 shows the mean percent templated edits at the HEK3 locus or at the VEGFA locus as encoded by 3′-extensions in Huh7 cells using MCP-PolK-nCas9 in the presence of an ngRNA.

FIG. 15 shows the percent indels at the HEK3 locus in Huh7 cells using SpyCas9 mRNA and a tgRNA with or without an ngRNA, wherein tgRNAs with varying numbers of 2′-O-methyl chemical modifications in the 3′-extension were used.

FIG. 16 shows the percent G-to-C editing at the HEK3 locus using tgRNAs comprising 3′ extensions with different DNA/RNA compositions, in the absence of ngRNA.

FIG. 17 shows the percent G-to-C editing at the HEK3 locus using tgRNAs comprising 3′ extensions with different DNA/RNA compositions, in the presence of ngRNA (G025992; SEQ ID NO: 481).

FIG. 18 shows G-to-C editing at the HEK3 locus in the absence of ngRNA (G025992) 3 days post LNP treatment with a guide RNA comprising a chimeric RNA/DNA 3′ extension and a mRNA encoding different nCas9-polymerase fusion proteins.

FIG. 19A shows G-to-C editing at the HEK3 locus in the absence of ngRNA (G025992) 6 days post LNP treatment with a guide RNA comprising a chimeric RNA/DNA 3′ extension and a mRNA encoding different nCas9-polymerase fusion proteins.

FIG. 19B shows G-to-C editing at the HEK3 locus in the presence of ngRNA (G025992) 3 days post LNP treatment with a guide RNA comprising a chimeric RNA/DNA 3′ extension and a mRNA encoding different nCas9-polymerase fusion proteins.

FIG. 20A shows G-to-C editing at the HEK3 locus in the presence of ngRNA (G025992) 6 days post LNP treatment with a guide RNA comprising a chimeric RNA/DNA 3′ extension and a mRNA encoding different nCas9-polymerase fusion proteins.

FIG. 20B shows G-to-C editing at the HEK3 locus in the absence of ngRNA (G025992) using MessengerMax™ delivery of a guide RNA comprising a chimeric RNA/DNA 3′ extension and a mRNA encoding different nCas9-polymerase fusion proteins.

FIG. 21 shows G-to-C editing at the HEK3 locus in the presence of ngRNA (G025992) using MessengerMax™ delivery of a guide RNA comprising a chimeric RNA/DNA 3′ extension and a mRNA encoding different nCas9-polymerase fusion proteins.

FIG. 22 shows G-to-C editing at the HEK3 locus in the absence of ngRNA, with DNA dependent polymerases using guides containing different DRS and template lengths.

FIG. 23 shows G-to-C editing at the HEK3 locus in the presence of ngRNA, with DNA dependent polymerases using guides containing different DRS and template lengths.

FIG. 24 shows the percentage of G-to-C editing at the HEK3 locus achieved with various ratios of mRNA to tgRNA.

FIG. 25 shows the percent G-to-C editing at the HEK3 locus obtained with nCas9-DNA polymerase fusion proteins with different T5 variants and a tgRNA (G031157).

FIG. 26 shows the percent G-to-C editing at the HEK3 locus using nCas9-DNA polymerase fusion proteins with T5 variant moieties and a tgRNA (G031157) and a nicking guide RNA (G025992).

FIG. 27 shows the percent G-to-C editing at the HEK3 locus using nCas9-DNA polymerase fusion proteins with T5 variant moieties and guides with varied template and DRS lengths.

FIG. 28 shows template-based editing at the HEK3 locus in Huh7 cells using mRNAs encoding different nCas9-polymerase fusion proteins and guide RNAs with 3′ extension sequences composed of RNA and DNA bases.

FIG. 29 shows template-based editing at the HEK3 locus in Huh7 cells using mRNAs encoding different nCas9-polymerase fusion proteins and guide RNAs with 3′ extension sequences composed of RNA and DNA bases.

FIG. 30 shows template-based editing at the HEK3 locus in Huh7 cells using mRNAs encoding different nCas9-polymerase fusion proteins and guide RNAs with 3′ extension sequences composed of RNA and DNA bases

FIG. 31 shows template-based editing at the HEK3 locus in Huh7 cells using different combinations of tgRNAs and mRNAs encoding different nCas9-polymerase fusion proteins.

FIG. 32 shows editing efficiency at the HEK3 locus in Huh7 cells using mRNAs encoding different nCas9-polymerase fusion proteins and guide RNAs with hybrid RNA/DNA 3′ extensions having different template and DRS lengths.

FIG. 33 shows editing efficiency at the HEK3 locus in Huh7 cells using mRNAs encoding different nCas9-polymerase fusion proteins and guide RNAs with hybrid RNA/DNA 3′ extensions having different template and DRS lengths, in the presence of ngRNA.

FIG. 34 shows editing efficiency at the HEK3 locus in HEK293T cells using mRNAs encoding different nCas9-polymerase fusion proteins and guide RNAs with hybrid RNA/DNA 3′ extensions having different template and DRS lengths.

FIG. 35 shows editing efficiency at the HEK3 locus in HEK293T cells using mRNAs encoding different nCas9-polymerase fusion proteins and guide RNAs with hybrid RNA/DNA 3′ extensions having different template and DRS lengths, in the presence of ngRNA.

FIG. 36 shows template-based editing at the HEK3 locus in Huh7 cells using mRNAs encoding different nCas9-polymerase fusion proteins.

FIG. 37 shows template-based editing at the HEK3 locus in Huh7 cells using mRNAs encoding different nCas9-polymerase fusion proteins.

FIGS. 38A and 38B show template-based editing at the HEK3 locus in HEK293T cells using mRNAs encoding different nCas9-polymerase fusion proteins in the absence (FIG. 38A) or presence (FIG. 38B) of ngRNA.

FIG. 39 shows template-based editing at the VEGFA locus in human CD34+ cells using different amounts of mRNA encoding nCas9-POLK (GE411-412RV), with or without a ngRNA.

FIG. 40 shows template-based editing at the HEK3 locus in Huh7 cells using mRNAs encoding different nCas9-polymerase fusion proteins and a guide RNA with a 3′ RNA extension.

FIG. 41 shows template-based editing at the VEGFA locus in Huh7 cells using mRNAs encoding different nCas9-polymerase fusion proteins and a guide RNA with a 3′ RNA extension.

FIG. 42 shows template-based editing at the VEGFA locus in primary human hepatocytes using mRNAs encoding different nCas9-polymerase fusion proteins and a guide RNA with a 3′ RNA extension.

FIG. 43 shows templated G-to-C editing at the HEK3 locus in Huh7 cells using (1) mRNAs encoding different polymerase-aptamer binding domain (ABD) fusion proteins, (2) mRNAs encoding nCas9, (3) an sgRNA, and (4) a separable 3′ extension comprising an aptamer.

FIG. 44 shows the percent indels at the HEK3 locus in Huh7 cells using (1) mRNAs encoding different polymerase-ABD fusion proteins, (2) mRNAs encoding nCas9, (3) an sgRNA, and (4) a separable 3′ extension comprising an aptamer.

FIG. 45A shows the editing efficiency at the VEGFA locus in Huh7 treated with an mRNA encoding MCP-PolK-nCas9, an sgRNA, and a separable MS2-loop-linked 3′ extension encoding a point correction at various positions of the template, with or without an ngRNA.

FIG. 45B shows the indel analysis at the VEGFA locus in Huh7 cells treated with SpyCas9 mRNA, a sgRNA, and a separable MS2-loop-linked 3′ extension encoding a point correction at various positions of the template.

FIG. 46A shows editing efficiency at the VEGFA locus in Huh7 cells treated with an mRNA encoding MCP-PolK-nCas9, an sgRNA, and separable MS2-loop-linked 3′ extensions encoding insertion edits of various lengths, with or without ngRNA.

FIG. 46B shows the indel analysis at the VEGFA locus in Huh7 cells treated with SpyCas9 mRNA, a sgRNA, and a separable MS2-loop-linked 3′ extension encoding insertion edits of various lengths.

FIG. 47A shows editing efficiency at the VEGFA locus in Huh7 cells treated with an mRNA encoding MCP-PolK-nCas9, an sgRNA, and separable MS2-loop-linked 3′ extensions encoding deletion edits of various lengths, with or without ngRNA.

FIG. 47B shows the indel analysis at the VEGFA locus in Huh7 cells treated with SpyCas9 mRNA, a sgRNA, and a separable MS2-loop-linked 3′ extension encoding deletion edits of various lengths.

FIG. 48A shows templated editing at the VEGFA locus in Huh7 cells treated with an mRNA encoding MCP-PolK-nCas9, an sgRNA, and separable MS2-loop-linked 3′ extensions differing in the number and position of MS2 loops and internal linkers, with or without ngRNA.

FIG. 48B shows the indel analysis at the VEGFA locus in Huh7 cells treated with SpyCas9 mRNA, a sgRNA, and a separable MS2-loop-linked 3′ extension differing in the number and position of MS2 loops and internal linkers.

FIG. 49A shows editing efficiency at the VEGFA locus in Huh7 cells treated with an mRNA encoding MCP-PolK-nCas9, an sgRNA, and separable MS2-loop-linked 3′ extensions encoding the GAGG>CACT edit with an additional mismatch in the DRS at varying positions, with or without ngRNA.

FIG. 49B shows the indel analysis at the VEGFA locus in Huh7 cells treated with an mRNA encoding SpyCas9, an sgRNA, and separable MS2-loop-linked 3′ extensions encoding the GAGG>CACT edit with an additional mismatch in the DRS at varying positions.

FIG. 50 shows templated editing at the VEGFA locus in primary human hepatocytes treated with an nCas9-polymerase fusion protein, (2) chemically modified tgRNAs, and (3) Vpx.

FIG. 51 shows the indel analysis at the VEGFA locus in primary human hepatocytes treated with SpyCas9, (2) chemically modified tgRNAs, and (3) Vpx.

FIG. 52 shows the editing efficiency at the VEGFA locus in primary human hepatocytes treated with an nCas9-polymerase fusion protein, (2) chemically modified tgRNAs, (3) Vpx, and (4) FEN1.

FIG. 53 shows the indel analysis at the VEGFA locus in primary human hepatocytes treated with SpyCas9, (2) chemically modified tgRNAs, (3) Vpx, and (4) FEN1.

FIG. 54 shows the editing efficiency at the VEGFA locus in primary human hepatocytes treated with an nCas9-polymerase fusion protein, (2) a tgRNA, and (3) Vpx.

FIG. 55 shows the editing efficiency at the VEGFA locus in primary human hepatocytes treated with an nCas9-polymerase fusion protein, (2) a tgRNA and (3) FEN1.

FIG. 56 shows the editing efficiency at the VEGFA locus in primary human hepatocytes treated with an nCas9-polymerase fusion protein, (2) a tgRNA and (3) MLH1-dn.

FIG. 57 shows the editing efficiency at the VEGFA locus in primary human hepatocytes treated with an nCas9-polymerase fusion protein, (2) a tgRNA and (3) MLH1-NTD.

FIG. 58 shows the editing efficiency at the VEGFA locus in primary human hepatocytes treated with an nCas9-polymerase fusion protein, (2) a tgRNA, and (3) Vpx, FEN1, or a combination of Vpx and FEN1.

FIG. 59 shows the editing efficiency at the VEGFA locus in primary human hepatocytes treated with an nCas9-polymerase fusion protein, (2) a tgRNA, and (3) Vpx, FEN1, or a combination of Vpx and FEN1.

FIG. 60 shows the editing efficiency at the VEGFA locus in Huh7 cells treated with an nCas9-polymerase fusion protein.

FIG. 61 shows the editing efficiency at the VEGFA locus in primary human hepatocytes treated with an nCas9-polymerase fusion protein.

FIG. 62 shows templated editing at the VEGFA locus in primary human hepatocytes treated with one of hFEN1 or hFEN1 w/o heterologous NLS, in combination with nCas9-T5pol(v3).

FIG. 63 shows templated editing at the VEGFA locus in primary human hepatocytes treated with one of hFEN1, cynoFEN1, or pictaFEN1, in combination with nCas9-T5pol(v3).

FIG. 64 shows templated editing at the VEGFA locus in primary human hepatocytes treated with one of hFEN1, hFEN1-KI, or hFEN1-EI, in combination with one of nCas9-T5pol(v3), nCas9-T5-EI, or nCas9-T5-KI.

FIG. 65 shows templated editing at the VEGFA locus in primary human hepatocytes treated with one of hFEN1, hFEN1-KI, or hFEN1-EI, along with one of nCas9-T5pol(v3), nCas9-T5-EI, or nCas9-T5-KI.

FIG. 66 shows the editing efficiency at the VEGFA locus in primary human hepatocytes treated with nCas9-T5pol(v3) in the presence of serial dilutions of hFEN1 and Vpx.

FIG. 67A shows the editing efficiency at the VEGFA locus in primary human hepatocytes treated with nCas9-T5-KI in the presence of serial dilutions of hFEN1-EI and Vpx.

FIG. 67B shows the editing efficiency at the VEGFA locus in primary human hepatocytes treated with nCas9-T5-EI in the presence of serial dilutions of hFEN1-KI and Vpx.

FIG. 68 shows the editing efficiency at the VEGFA locus in primary human hepatocytes treated with nCas9-T5pol(v3) in the presence of serial dilutions of hFEN1 and M97.

FIG. 69A shows the editing efficiency at the VEGFA locus in primary human hepatocytes treated with nCas9-T5-KI in the presence of hFEN1-EI and M97.

FIG. 69B shows the editing efficiency at the VEGFA locus in primary human hepatocytes treated with nCas9-T5-KI in the presence of hFEN1-EI and M97.

FIG. 70 shows the editing at the VEGFA locus in primary human hepatocytes treated with an nCas9-polymerase fusion protein, Vpx and FEN1, and in huh7 cells treated with an nCas9-polymerase fusion protein and MLH1-dn.

FIG. 71A shows editing in 16HBE cells treated with SpyCas9 or a fusion protein comprising a SpyCas9 nickase moiety and a polymerase moiety.

FIG. 71B shows indel in 16HBE cells treated with SpyCas9 or a fusion protein comprising a SpyCas9 nickase moiety and a polymerase moiety.

FIGS. 72A and 72B show editing using guides designs varying the position, number and chemical modifications on or surrounding the MS2-loop feature treated with the MCP-PolK-nCas9 or nCas9-T5Pol(D164A,E166A)-MCP construct, respectively.

FIGS. 73A and 73B show editing using guide designs varying the number of phosphorothioate bonds treated with the MCP-PolK-nCas9 or nCas9-T5Pol(D164A,E166A)-MCP construct, respectively.

FIGS. 74A and 74B show editing using guide designs varying the position of phosphorothioate bonds treated with the MCP-PolK-nCas9 or nCas9-T5Pol(D164A,E166A)-MCP construct, respectively.

FIGS. 75A and 75B show editing using guide designs with varying number of nucleotides with a 2′ O-methyl modification treated with the MCP-PolK-nCas9 or nCas9-T5Pol(D164A,E166A)-MCP construct, respectively.

FIGS. 76A and 76B show editing using guide designs varying the position of 2′ O-methyl modification treated with the MCP-PolK-nCas9 or nCas9-T5Pol(D164A,E166A)-MCP construct, respectively.

FIGS. 77A and 77B show editing using guide designs incorporating deoxynucleotides at various positions relative to the template:DRS junction treated with the MCP-PolK-nCas9 or nCas9-T5Pol(D164A,E166A)-MCP construct, respectively.

FIGS. 78A and 78B show editing using guide designs with varying template lengths, varying DRS lengths, or the incorporation of deoxyuracil at select positions treated with the MCP-PolK-nCas9 or nCas9-T5Pol(D164A,E166A)-MCP construct, respectively.

FIGS. 79A-79C show editing at the VEGFA locus using various guide designs with nCas9-T5Pol(D164A,E166A) (FIG. 79A), nCas9-polK (FIG. 79B), and SpyCas9 (FIG. 79C).

FIGS. 80A-80B show templated editing at the TTR locus in PCH cells treated with nCas9-T5pol(v3) and a tgRNA (G037455) in the presence or absence of HIV-Vpx-1, SIV-Vpx, M97, or MLH1 (FIG. 80A), or huFEN1 or cynoFEN1 (FIG. 80B).

FIG. 81 shows templated editing at the TTR locus in PCH cells treated with a tgRNA (G037455), nCas9-T5pol(v3) or nCas9-T5-EI, and FEN1 or FEN1-KI.

FIG. 82 shows templated editing at the TTR locus in PCH cells treated with nCas9-T5pol(v2), FEN1 and Vpx, and different concentrations of a tgRNA (G037455).

FIG. 83 shows templated editing at the DNMT1 locus in mice treated with an mRNA encoding nCas9-PolK(1-526, GE411-412RV), angRNA (G035722), and a tgRNA (G034987; SEQ ID NO: 427), delivered via LNPs with different formulations.

FIG. 84 shows a Western blot of rat liver protein lysates immunostained with anti-SpyCas9 antibodies. Liver tissue was collected 2.5 hours after in vivo delivery in trans of an mRNA encoding a SpyCas9 nickase fused to a split intein, and an mRNA encoding a polymerase fused to a complementary split intein.

FIG. 85A shows editing at the TTR locus in PCH treated with a tgRNA (G036237; SEQ ID NO: 477), along with nCas9-PolK(1-526, GE411-412RV), or a combination of nCas9-CfaN and Cfa-PolK(1-526, GE411-412RV), in the presence of Vpx.

FIG. 85B shows editing at the TTR locus in PCH treated with a tgRNA (G036237), along with nCas9-T5pol(v4), or a combination of nCas9-CfaN and Cfa-T5pol(v4), in the presence of Vpx.

FIG. 86 shows templated editing at the DNMT1 locus in PMH treated with a tgRNA (G034987), along with nCas9-PolK(1-526, GE411-412RV) or a combination of nCas9-CfaN and Cfa-PolK(1-526, GE411-412RV) in a nCas9-CfaN:CfaC-PolK(1-526, GE411-412RV) mass ratio of 5:1, 3:1, 1:1, or 1:3.

FIG. 87 shows templated editing at the DNMT1 locus in PMH treated with a tgRNA (G034987), along with (i) nCas9-T5(D164A, E166A), (ii) a combination of nCas9-CfaN and CfaC-L30(SV40)-T5(D164A, E166A) in a 5:1 or 1:1 mass ratio, or (iii) a combination of nCas9-CfaN and CfaC-L49(SV40)-T5(D164A, E166A) in a 5:1 or 1:1 mass ratio.

FIGS. 88A and 88B show editing at the TTR locus in PCH treated with a tgRNA (G039268), along with (i) nCas9-PolKv2, (ii) nCas9-T5(D164A, E166A, (iii) a combination of nCas9-CfaN and Cfa-PolK(1-526, GE411-412RV) at a 5:1 mass ratio, (iv) a combination of Spy(2-713)-CfaN with either one of CfaC-Spy(714-1368)-PolKvariant or CfaC-Spy(714-1368)-T5polvariant, at a 1:1 mass ratio, or (v) a combination of HMGB1dT-Spy(2-713)-CfaN and CfaC-Spy(714-1368)-PolKvariant at a 1:1 mass ratio, in the presence of Vpx. In the context of FIG. 88, “HMGBIdt” indicates an HMGB1 polypeptide comprising the HMGB1 Box A domain and Box B domain and lacking an acidic tail domain (“HMGB1_dTail”).

FIG. 89 shows templated editing at the DNMT1 locus in mice treated with a fusion protein comprising a nCas9 moiety and a polymerase Kappa variant moiety, or a combination of a nCas9-CfaN fusion protein and a CfaC-PolK(1-526, GE411-412RV), along with a tgRNA (G034987), and a ngRNA (G035727; SEQ ID NO: 428).

FIG. 90 shows editing efficiencies at the DNMT1 locus in mice treated with a combination of a nCas9-CfaN fusion protein and a CfaC-PolK(1-526, GE411-412RV), along with a tgRNA (G034987), and a ngRNA (G035727).

FIG. 91 shows editing efficiencies obtained at the VEGFA locus in PHH treated with a tgRNA (G032596; SEQ ID NO: 417), along with split intein constructs comprising a nCas9 and a polymerase delivered in trans, with editing enhancers delivered in trans or in cis with the split intein constructs.

FIG. 92 shows editing efficiencies obtained at the TTR locus in PCH treated with tgRNA (G039267) and a ngRNA (G032630), along with split intein constructs comprising a nCas9 and a polymerase delivered in trans, with editing enhancers delivered in trans or in cis with the split intein constructs.

FIG. 93 shows editing efficiencies obtained at the VEGFA locus in PHH treated with a tgRNA (G032596), along with split intein constructs comprising a nCas9 fused with an HMGB1 polypeptide, and a polymerase delivered in trans, with editing enhancers delivered in trans or in cis with the split intein constructs. In the context of FIG. 93, “HMGB1dt” indicates an HMGB1 polypeptide comprising the HMGB1 Box A domain and Box B domain and lacking an acidic tail domain (alternatively referred elsewhere herein as “HMGB1_dTail”).

FIG. 94 shows editing efficiencies obtained at the TTR locus in PCH treated with a tgRNA (G039267) and a ngRNA (G032630), along with split intein constructs comprising a nCas9 fused with a HMGB1 polypeptide and a polymerase delivered in trans, with editing enhancers delivered in trans or in cis with the split intein constructs. In the context of FIG. 94, “HMGB1dt” indicates an HMGB1 polypeptide comprising the HMGB1 Box A domain and Box B domain and lacking an acidic tail domain (“HMGB1_dTail”).

FIGS. 95A and 95B shows templated editing at the HEK3 locus in PHH for cells treated with G032574 (SEQ ID NO: 528), along with nCas9-PolK(1-526, GE411-412RV), HMGB1dT-nCas9-PolK(1-526, GE411-412RV), nCas9-T5pol(v2), or HMGBldT-nCas9-T5pol(v2), in the absence or presence of Vpx. In the context of FIG. 95, “HMGB1dt” indicates an HMGB1 polypeptide comprising the HMGB1 Box A domain and Box B domain and lacking an acidic tail domain (“HMGB1_dTail”).

FIG. 96 shows templated editing at the VEGFA locus in huh7 cells treated with a mRNA encoding a nCas9-polymerase fusion protein selected from nCas9-PolKv2, nCas9-T5 (WT), or nCas9-T5(D164A, E166A), and with tgRNAs encoding a GAGG-to-CACT edit, and comprising different dNTP/rNTP compositions at the template:DRS junction.

FIGS. 97A and 97B shows editing at the VEGFA locus in huh7 cells treated with a mRNA encoding a nCas9-polymerase fusion (nCas9-PolKv2 or nCas9-T5(D164A, E166A)) and with tgRNAs encoding a GAGG-to-CACT edit, and comprising different dNTP/rNTP compositions in the DRS region.

FIG. 98 shows editing at the VEGFA locus in huh7 cells treated with a mRNA encoding a nCas9-polymerase fusion (nCas9-PolKv2 or nCas9-T5(D164A, E166A)) and with tgRNAs encoding a GAGG-to-CACT edit, and comprising different dNTP/rNTP compositions and 2′Ome modifications in the spacer sequence.

FIG. 99 shows editing at the VEGFA locus in huh7 cells treated with a mRNA encoding a nCas9-polymerase fusion protein selected from nCas9-PolKv2, nCas9-T5 (WT), or nCas9-T5(D164A, E166A), and with tgRNAs encoding a GAGG-to-CACT edit, and comprising different dNTP/rNTP compositions in the DRS region, and various modifications of the nucleotides in the spacer region.

FIGS. 100A and 100B show the editing results at the VEGFA locus in PHH treated with tgRNAs comprising different dNTP/rNTP compositions at the template:DRS junction, along with nCas9-PolKv2 (FIG. 100A) or nCas9-T5(D164A, E166A) (FIG. 100B).

FIGS. 101A and 101B show the editing results at the VEGFA locus in PHH treated with tgRNAs comprising different dNTP/rNTP compositions in the DRS region, along with nCas9-PolKv2 (FIG. 101A) or nCas9-T5(D164A, E166A) (FIG. 101B).

FIGS. 102A and 102B show the editing results at the VEGFA locus in PHH treated with tgRNAs comprising different dNTP/rNTP compositions and 2′Ome modifications in the spacer sequence, along with nCas9-PolKv2 (FIG. 102A) or nCas9-T5(D164A, E166A) (FIG. 102B).

FIGS. 103A-103B show templated editing at the TTR locus in PCH treated with nCas9-T5pol(v3) or nCas9-PolK(1-526, GE411-412RV), in the presence of Vpx and FEN-1, and with tgRNAs designed with the scaffold structure shown in FIG. 6F, and with different template lengths.

FIG. 104 shows editing at the TTR locus in PCH treated with nCas9-T5pol(v2), in the presence of Vpx and FEN-1, and with tgRNAs designed with the scaffold structure shown in FIG. 6F, and with different template lengths.

FIGS. 105A-105B show templated editing at the TTR locus in PCH treated with nCas9-T5pol(v2) or nCas9-PolK(1-526, GE411-412RV), in the presence of Vpx and FEN-1, and with tgRNAs designed with the scaffold structure shown in FIG. 6F, and with different template lengths.

FIG. 106A shows a one-site guide ligation approach to produce a tgRNA by ligating two split fragments of the desired final tgRNA product.

FIG. 106B shows a one-step two-site guide ligation approach to produce a tgRNA by ligating three split fragments of the desired final tgRNA product.

FIG. 106C shows a one-step two-site guide ligation approach compared to a one-site guide ligation approach for the same 179-nt desired ligated tgRNA product (LS=ligation site). RNA nucleotides are indicated in black text, DNA nucleotides are indicated in gray text, 2′-O-Me modified nucleotides are shown in circles and phosphorothioate (PS) linkages are shown with “*”.

FIG. 107 shows a gel analysis of the elute mix from one batch following purification by precipitation.

FIG. 108 shows a UPLC-UV chromatogram of the elute batches obtained after purification by precipitation.

FIG. 109 shows a UPLC-UV chromatogram of the final ligated tgRNA product following RP-HPLC purification.

FIG. 110 shows gel analysis of the resuspended solution following purification by precipitation.

FIG. 111 shows a UPLC-UV chromatogram obtained after purification by precipitation.

FIG. 112 shows an HPLC-UV chromatogram showing the fractionation and sample pools obtained during the purification of the ligated tgRNA product (FLP).

FIG. 113 shows a UPLC-UV chromatogram of the final ligated tgRNA product after purification by HPLC.

FIG. 114 shows a gel analysis of the resuspended solution following purification by precipitation.

FIG. 115 shows a UPLC-UV chromatogram obtained after purification by precipitation.

FIG. 116 shows an HPLC-UV chromatogram showing the fractionation and sample pools obtained during the purification of the ligated tgRNA product (FLP).

FIG. 117 shows a UPLC-UV chromatogram of the final ligated tgRNA product after purification by HPLC.

FIG. 118 shows a structure of wild-type HMGB1, including various domains therein.

FIG. 119 shows domains of exemplary fusion proteins comprising an HMGB1 polypeptide and a programmable DNA-binding protein domain. In the context of FIG. 114, “DNA binding domain” refers to a SpyCas9 nickase.

FIG. 120 shows domains of exemplary fusion proteins described herein.

### DETAILED DESCRIPTION

The present disclosure provides, e.g., compositions, methods, and systems for template-based genome editing. In some embodiments, the compositions, methods, and systems provide for generating templated insertions, deletions, or substitutions within the genome of a cell without the involvement of the HDR pathway.

In some embodiments, the compositions, methods, and systems disclosed herein comprise a SpyCas9 nickase, DNA-dependent DNA polymerase, and template guide RNA (tgRNA).

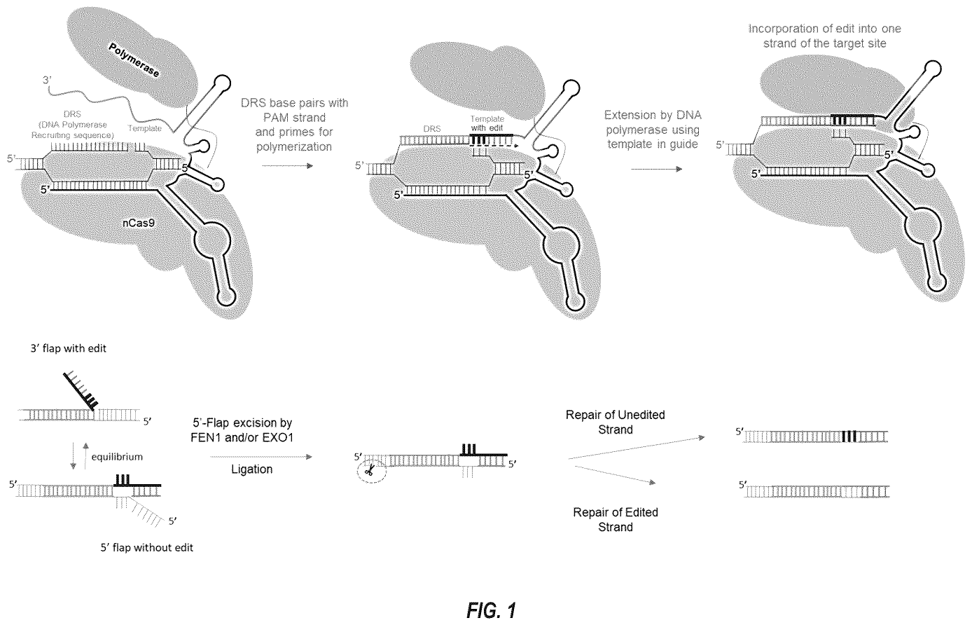

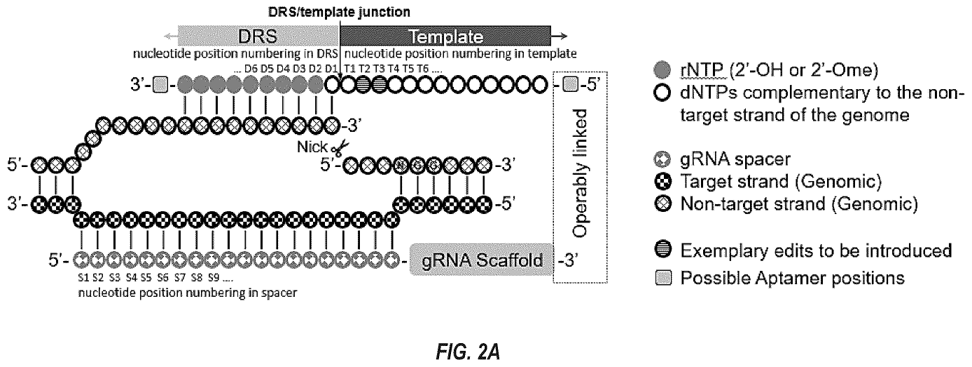

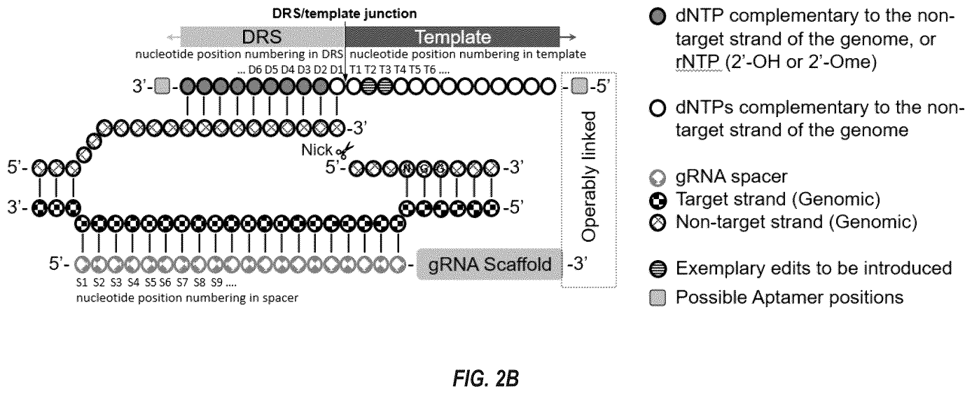

Without being bound by theory, an exemplary tgRNA according to the present disclosure may comprise a SpyCas9 guide RNA that is capable of binding to the SpyCas9 nickase (via a scaffold sequence) and a target region of a target strand of a DNA duplex target nucleic acid (via a spacer sequence) (see, e.g., FIG. 1 (top panel)). The SpyCas9 guide RNA thereby facilitates binding of the SpyCas9 nickase to the target region. The SpyCas9 nickase preferably comprises a substitution in the HNH nuclease domain (e.g., a H840A substitution). Once bound to the target region, the SpyCas9 nickase may generate a nick in a non-target strand of the DNA duplex target nucleic acid. The nicked strand of the DNA duplex target nucleic acid may comprise a flap 5′ of the nick (the 3′ end flap) and a flap 3′ of the nick (the 5′ end flap) (see FIGS. 2A and 2B, which show the 3′ end of the flap 5′ of the nick and the 5′ end of the flap 3′ of the nick).

An exemplary tgRNA according to the present disclosure may further comprise a 3′ extension comprising (from 5′ to 3′) a template sequence and a DNA polymerase-recruiting sequence (DRS). The DRS may be designed to hybridize to the 3′ end of the flap 5′ of the nick in the non-target strand of the DNA duplex target nucleic acid, thereby forming a duplex (see, e.g., FIGS. 1 (top panel), 2A, and 2B). Subsequently, the DNA-dependent DNA polymerase may bind to the duplex formed by the DRS and 3′ end of the flap 5′ of the nick. The 3′ end of the flap 5′ of the nick may serve as a primer for synthesis of a new DNA strand that is complementary to the template sequence. The template sequence may include one or more of an insertion, deletion, or substitution (e.g., a transition or a transversion) relative to an endogenous nucleotide sequence. For example, FIGS. 2A and 2B show an exemplary template comprising a two-nucleotide substitution relative to the endogenous nucleotide sequence. Following DNA synthesis, the newly synthesized DNA strand may include one or more of an insertion, deletion, or substitution relative to the endogenous nucleotide sequence (see FIG. 1, which shows the newly synthesized DNA strand with an edit relative to the endogenous DNA sequence). As shown in FIG. 1 (bottom panel), the newly synthesized DNA strand and the flap 3′ of the nick may competitively bind to the target strand. The flap 3′ of the nick may be excised, for example by FEN1 or EXO1, and the newly synthesized DNA strand may anneal to the target strand. Subsequent mismatch repair may result in either (1) repair of the unedited strand using the newly synthesized DNA strand, thereby incorporating the insertion, deletion, or substitution into the genomic sequence; or (2) repair of the edited strand using the unedited strand, thereby preserving the original sequence.

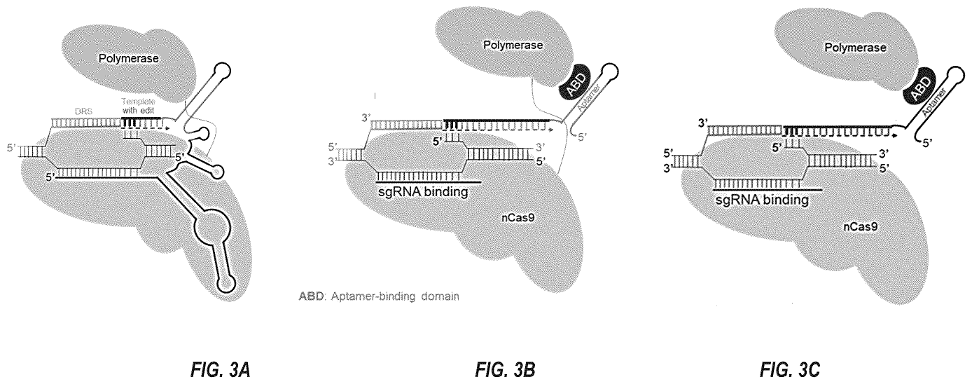

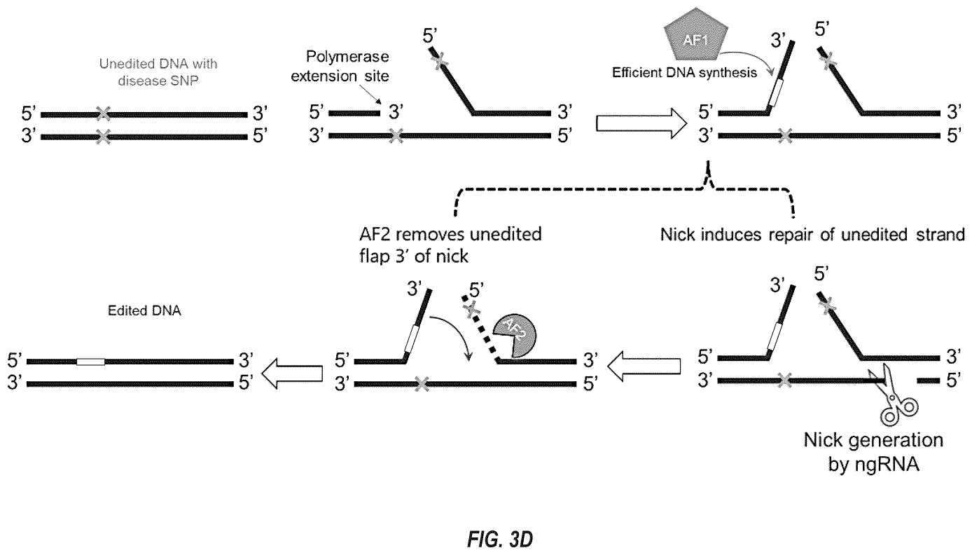

As shown in FIG. 3D, the compositions and methods provided herein further may comprise one or more enhancers of template-based genome editing. In some embodiments, such enhancers may promote DNA synthesis by increasing the concentration of dNTPs within the cell, e.g., by inhibiting a deoxynucleotide triphosphohydrolase protein (see AF1 in FIG. 3D). In some embodiments, such enhancers may promote the hybridization of the newly synthesized DNA strand and the target strand. For example, DNA repair proteins may remove the flap 3′ of the nick (see AF2 in FIG. 3D). Removal of the flap 3′ of the nick may promote hybridization of the newly synthesized DNA strand to the target strand of the DNA duplex target nucleic acid.

Compositions comprising (1) a SpyCas9 nickase, (2) an RNA-dependent DNA polymerase (e.g., a reverse transcriptase), and (3) a guide RNA comprising an RNA-based template sequence have previously been shown to install templated edits into genomic sequences. However, such compositions and methods can produce unintended genetic modifications. Thus, the utility of such compositions and methods is currently limited. For example, after synthesizing a new DNA strand that is complementary to the template sequence, RNA-dependent DNA polymerases may continue DNA synthesis, using the scaffold of the SpyCas9 guide RNA as a template and thereby creating unintended modifications whereby a portion of the scaffold sequence is inadvertently incorporated into the DNA. Accordingly, there is a need for compositions and methods that do not run the risk of incorporating the SpyCas9 guide RNA scaffold sequence into the newly sequenced DNA strand.

Exemplary compositions according to the present disclosure comprise DNA-dependent DNA polymerases instead of RNA-dependent DNA polymerases. DNA-dependent DNA polymerases such as T5 polymerase and PolK polymerase have a strong preference for DNA templates, and may therefore exhibit a reduced propensity to continue DNA synthesis past a DNA-RNA boundary, such as the boundary between a template sequence comprising a plurality of DNA nucleotides and a SpyCas9 guide RNA scaffold sequence comprising RNA nucleotides.

Additionally, there is a need for enzymes (e.g., SpyCas9 nickases and DNA-dependent DNA polymerases) with robust templated editing activity at physiological temperatures, e.g., 37° C. Additionally, the production or synthesis of sufficiently pure polynucleotides (e.g., tgRNAs) over 110 nucleotides in length has proved challenging. Thus, there is a need for methods of producing or synthesizing such polynucleotides.

In some embodiments, the present disclosure provides for a system comprising a DNA-dependent DNA polymerase, a SpyCas9 nickase (nCas9), and a template guide RNA (tgRNA), wherein the tgRNA comprises a first polynucleotide and a second polynucleotide, wherein: (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; (ii) a DNA-dependent DNA polymerase recruiting sequence (DRS) that is 6-20 nucleotides in length, optionally 6-17 or 10-14 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid. In some embodiments, the DRS of the tgRNA comprises DNA nucleotides. In some embodiments, the DRS of the tgRNA comprises both RNA nucleotide(s) and DNA nucleotide(s).

In some embodiments, the present disclosure provides for a system comprising a DNA-dependent DNA polymerase, a SpyCas9 nickase (nCas9), and a template guide RNA (tgRNA), wherein the tgRNA comprises a first polynucleotide and a second polynucleotide, wherein: (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; (ii) a DNA-dependent DNA polymerase recruiting sequence (DRS) that is 6-20 nucleotides in length, optionally 6-17 or 10-14 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid, the DRS comprises DNA nucleotides; and wherein the DRS comprises at least one of: (1) a sequence not fully complementary to the DRS-complementary region of the non-target strand of the DNA duplex target nucleic acid; (2) a 1-5 nucleotide, optionally a 1-3 nucleotide 3′ terminal tail comprising a 3′ terminal modified uridine nucleotide; or (3) a 5′ terminal (D1) DNA nucleotide and a penultimate 5′ terminal (D2) RNA nucleotide wherein the 3′ end of the first polynucleotide is covalently linked to the 5′ end of the second polynucleotide by a phosphorothioate or phosphodiester linkage.

In some embodiments, the present disclosure provides for a system comprising a DNA-dependent DNA polymerase, a SpyCas9 nickase (nCas9), and a template guide RNA (tgRNA), wherein the tgRNA comprises a first polynucleotide and a second polynucleotide, wherein: (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; (ii) a DNA-dependent DNA polymerase recruiting sequence (DRS) that is 6-20 nucleotides in length, optionally 6-17 or 10-14 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid, wherein the DRS comprises RNA nucleotides; and wherein the DRS comprises at least one of: (1) a sequence not fully complementary to the DRS-complementary region of the non-target strand of the DNA duplex target nucleic acid; (2) a 1-5 nucleotide, optionally a 1-3 nucleotide 3′ terminal tail comprising a 3′ terminal modified uridine nucleotide; or (3) a 5′ terminal (D1) DNA nucleotide and a penultimate 5′ terminal (D2) RNA nucleotide wherein the 3′ end of the first polynucleotide is covalently linked to the 5′ end of the second polynucleotide by a phosphorothioate or phosphodiester linkage.

In some embodiments, the present disclosure provides for a system comprising a DNA polymerase kappa (polK), a SpyCas9 nickase (nCas9), and a template guide RNA (tgRNA), wherein the tgRNA comprises a first polynucleotide and a second polynucleotide, wherein (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; (ii) a DNA-dependent DNA polymerase recruiting sequence (DRS) that is about 6-18 nucleotides in length, optionally about 8-18 or 10-12 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid. In some embodiments, the DRS of the tgRNA comprises DNA nucleotides. In some embodiments, the DRS of the tgRNA comprises both RNA nucleotide(s) and DNA nucleotide(s).

In some embodiments, the present disclosure provides for a system comprising a DNA polymerase kappa (polK), a SpyCas9 nickase (nCas9), and a template guide RNA (tgRNA), wherein the tgRNA comprises a first polynucleotide and a second polynucleotide, wherein (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; (ii) a DNA-dependent DNA polymerase recruiting sequence (DRS) that is about 6-18 nucleotides in length, optionally about 8-18 or 10-12 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid, wherein the DRS comprises RNA nucleotides.

In some embodiments, the present disclosure provides for a system comprising a T5 DNA polymerase (T5 pol), a SpyCas9 nickase (nCas9), and a template guide RNA (tgRNA), wherein the tgRNA comprises a first polynucleotide and a second polynucleotide, wherein (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; and (ii) a DNA-dependent DNA polymerase recruiting sequence (DRS) that is 6-20 nucleotides in length, optionally 6-17 or 10-14 nucleotides in length, and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid comprising nucleotides 5-10 5′ of the PAM in the non-target strand of the DNA duplex target nucleic acid. In some embodiments, the DRS of the tgRNA comprises DNA nucleotides. In some embodiments, the DRS of the tgRNA comprises both RNA nucleotide(s) and DNA nucleotide(s).

In some embodiments, the present disclosure provides for a system comprising a T5 DNA polymerase (T5 pol), a SpyCas9 nickase (nCas9), and a template guide RNA (tgRNA), wherein the tgRNA comprises a first polynucleotide and a second polynucleotide, wherein (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; and (ii) a DNA-dependent DNA polymerase recruiting sequence (DRS) that is 6-20 nucleotides in length, optionally 6-17 or 10-14 nucleotides in length, and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid comprising nucleotides 5-10 5′ of the PAM in the non-target strand of the DNA duplex target nucleic acid, wherein the DRS comprises RNA nucleotides.

In some embodiments, the first polynucleotide and the second polynucleotide are covalently linked. In some embodiments, the first polynucleotide and the second polynucleotide are not covalently linked. In some embodiments, the template guide RNA further comprises an affinity tag, wherein the first polynucleotide or the second polynucleotide are bound to a peptide or a peptide complex comprising a SpyCas9 nickase, and a DNA-dependent DNA polymerase by the affinity tag.

In some embodiments, the composition further comprises a SpyCas9 nickase. In some embodiments, the scaffold of the SpyCas9 guide RNA binds to a SpyCas9 nickase. In some embodiments, the SpyCas9 nickase generates a nick on the same strand of the DNA duplex target nucleic acid as a nickase comprising an H840A mutation, i.e., the SpyCas9 nickase cleaves the strand of the DNA duplex target nucleic acid opposite to the strand of the DNA duplex target nucleic acid that hybridizes to the spacer (i.e., the nickase cleaves the non-target strand of the DNA duplex target nucleic acid). In some embodiments, the SpyCas9 nickase generates a nick on the non-target strand of the DNA duplex target nucleic acid. In some embodiments, the SpyCas9 nickase comprises a catalytically inactive HINH domain. In some embodiments, the SpyCas9 nickase comprises a D839K, D839Q, D839N, D839A, H840S, H840T, H840Q, H840D, H840R, H840K, H840Y, H840N, N863S, N863T, N863A, N863E, or K866A mutation within its HNH-like nuclease domain. In some embodiments, the SpyCas9 nickase comprises an H840A point mutation. Throughout the present disclosure, it is to be understood that, when the term “point mutation” is used in the context of an amino acid sequence, it refers to a substitution of a single amino acid residue in the amino acid sequence.

In some embodiments, the composition further comprises a DNA-dependent DNA polymerase. In some embodiments, the DRS bound to the non-target strand of the DNA duplex target nucleic acid forms a binding site for DNA-dependent DNA polymerase. In some embodiments, the DNA-dependent DNA polymerase comprises a eukaryotic DNA-dependent DNA polymerase. In some embodiments, the DNA-dependent DNA polymerase comprises a mammalian DNA-dependent DNA polymerase. In some embodiments, the DNA-dependent DNA polymerase comprises a human DNA-dependent DNA polymerase. In some embodiments, the DNA-dependent DNA polymerase comprises a cynomolgus DNA-dependent DNA polymerase. In some embodiments, the DNA-dependent DNA polymerase comprises a mouse DNA-dependent DNA polymerase. In some embodiments, the DNA-dependent DNA polymerase comprises a bacterial DNA-dependent DNA polymerase. In some embodiments, the DNA-dependent DNA polymerase comprises an E. coli DNA polymerase. In some embodiments, the DNA-dependent DNA polymerase comprises a viral DNA-dependent DNA polymerase. In some embodiments, the DNA-dependent DNA polymerase comprises a phage DNA-dependent DNA polymerase. In some embodiments, the DNA-dependent DNA polymerase comprises human DNA polymerase K, cynomolgus DNA polymerase K, mouse DNA polymerase K, human DNA polymerase θ, human DNA polymerase N, E. coli DNA polymerase I, phage T5 DNA polymerase, or phage Phi29 DNA polymerase. In some embodiments, the DNA-dependent DNA polymerase comprises human DNA polymerase K. In some embodiments, the DNA-dependent DNA polymerase comprises phage T5 DNA polymerase. In some embodiments, the DNA-dependent DNA polymerase does not comprise phage Phi29 DNA polymerase. In some embodiments, the DNA-dependent DNA polymerase comprises a point mutation(s) or deletion(s). In some embodiments, the DNA-dependent DNA polymerase comprises a point mutation(s) or deletion(s) within an exonuclease domain. In some embodiments, the point mutation(s) or deletion(s) within the exonuclease domain abolishes exonuclease activity.

In some embodiments, the SpyCas9 nickase is operably linked to the DNA-dependent DNA polymerase. In some embodiments, the SpyCas9 nickase and DNA-dependent DNA polymerase comprise a single polypeptide. In some embodiments, the SpyCas9 nickase and DNA-dependent DNA polymerase comprise a single polypeptide expressed from a single open reading frame (ORF). In some embodiments, the SpyCas9 nickase and DNA-dependent DNA polymerase comprise a single polypeptide expressed from separate open reading frames (ORFs). In some embodiments, wherein the SpyCas9 nickase and DNA-dependent DNA polymerase comprise a single polypeptide expressed from separate open reading frames (ORFs), the ORF encoding the SpyCas9 nickase can further encode a first intein and the ORF encoding the DNA-dependent DNA polymerase can further encode a second intein capable of binding the first intein (i.e. participating in a trans-splicing reaction with the first intein). In some embodiments, the SpyCas9 nickase and DNA-dependent DNA polymerase are provided as separate polypeptides. In some embodiments, wherein the SpyCas9 nickase and DNA-dependent DNA polymerase comprise a single polypeptide expressed form separate open reading frames (ORFs), the ORF encoding the SpyCas9 nickase can further encode a first intein and the ORF encoding the DNA-dependent DNA polymerase can further encode a second intein capable of binding the first intein (i.e. participating in a trans-splicing reaction with the first intein). In some embodiments, the SpyCas9 nickase and DNA-dependent DNA polymerase are provided as separate polypeptides. In some embodiments, the SpyCas9 nickase and DNA-dependent DNA polymerase directly interact through non-covalent interactions. In some embodiments, the SpyCas9 nickase and DNA-dependent DNA polymerase do not directly interact. As a first example, in some embodiments, the SpyCas9 nickase and DNA-dependent DNA polymerase may both non-covalently bind a single polynucleotide such as a template guide RNA. As a second example, in some embodiments, the SpyCas9 nickase may bind a first polynucleotide comprising a guide RNA and the DNA-dependent DNA polymerase may bind a second polynucleotide comprising a separable 3′ extension, wherein the first and second polynucleotides target the SpyCas9 nickase and DNA-dependent DNA polymerase to proximal genomic loci.

In some embodiments, the composition comprises (a) a single polypeptide comprising a SpyCas9 nickase and a DNA-dependent DNA polymerase; and (b) a single polynucleotide comprising, from 5′ to 3′, (i) a SpyCas9 guide RNA comprising a spacer and a scaffold; and (ii) a template sequence and a DRS.

In some embodiments, the composition comprises (a) a single polypeptide comprising a SpyCas9 nickase and a DNA-dependent DNA polymerase; (b) a first polynucleotide comprising a SpyCas9 guide RNA comprising, from 5′ to 3′, a spacer and a scaffold; and (c) a second polynucleotide comprising, from 5′ to 3′, a template sequence and a DRS, wherein the first and second polynucleotide are provided as separate molecules, optionally wherein the second polynucleotide comprises an affinity tag, optionally an aptamer, for binding to the DNA-dependent DNA polymerase.

In some embodiments, the composition comprises (a) a first polypeptide comprising a SpyCas9 nickase; (b) a second polypeptide comprising a DNA-dependent DNA polymerase; and (c) a single polynucleotide comprising, from 5′ to 3′, (i) a SpyCas9 guide RNA comprising a spacer and a scaffold; and (ii) a template sequence and a DRS, wherein the first and second polypeptide are provided as separate molecules, optionally wherein the second polynucleotide comprises an affinity tag, optionally an aptamer, for binding to a 3′ extension-recruiting domain on the DNA-dependent DNA polymerase. In some embodiments, the SpyCas9 nickase and DNA-dependent DNA polymerase directly interact through non-covalent interactions. In some embodiments, the SpyCas9 nickase and DNA-dependent DNA polymerase do not directly interact. In some embodiments, the SpyCas9 nickase and DNA-dependent DNA polymerase may both non-covalently bind a single polynucleotide such as a template guide RNA.

In some embodiments, the composition comprises (a) a first polypeptide comprising a SpyCas9 nickase; (b) a second polypeptide comprising a DNA-dependent DNA polymerase and a 3′ extension-recruiting domain; (c) a first polynucleotide comprising a SpyCas9 guide RNA comprising, from 5′ to 3′, a spacer and a scaffold; and (d) a second polynucleotide comprising, from 5′ to 3′, a template sequence and a DRS, and further comprising an affinity tag; wherein the first and second polypeptide and first and second polynucleotide are provided as separate molecules. In some embodiments, the SpyCas9 nickase and DNA-dependent DNA polymerase directly interact through non-covalent interactions. In some embodiments, the SpyCas9 nickase and DNA-dependent DNA polymerase do not directly interact. In some embodiments, the SpyCas9 nickase may bind a first polynucleotide comprising a guide RNA and the DNA-dependent DNA polymerase may bind a second polynucleotide comprising a separable 3′ extension, wherein the first and second polynucleotides target the SpyCas9 nickase and DNA-dependent DNA polymerase to proximal genomic loci.

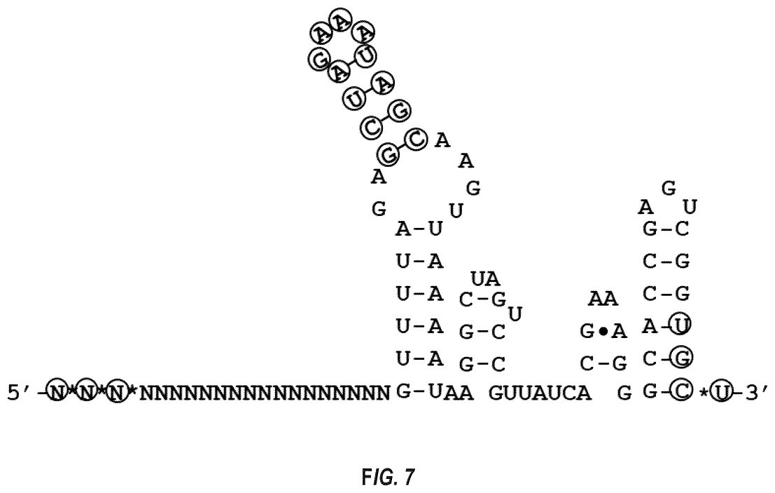

In some embodiments, the composition further comprises a nicking guide RNA (ngRNA; see, e.g., FIG. 7). The ngRNA comprises a spacer that hybridizes to a sequence such that the nick site of the ngRNA is within 200 nucleotides 5′ or 3′ from the nick site of the template guide RNA and on the target strand of the DNA duplex target nucleic acid, i.e., the strand to which the spacer of the tgRNA hybridizes. In other words, the ngRNA/SpyCas9 complex nicks the strand opposite to the strand nicked by the tgRNA/SpyCas9 complex. In some embodiments, the nick site of the ngRNA is about 20-200 nucleotides 5′ or 3′ from the nick site of the template guide RNA and on the target strand of the DNA duplex target nucleic acid. In some embodiments, the nick site of the ngRNA is within the genomic locus targeted by the spacer sequence of the template guide RNA. In some embodiments, the nick site of the ngRNA is preferably outside of the genomic locus targeted by the spacer sequence of the template guide RNA.

In some embodiments, the composition further comprises an enhancer of template-based genome editing. In some embodiments, the enhancer is an inhibitor of deoxynucleotide triphosphohydrolase. In some embodiments, wherein the enhancer is an inhibitor of deoxynucleotide triphosphohydrolase, the deoxynucleotide triphosphohydrolase comprises SAM domain and HD domain-containing protein 1 (SAMHD1). In some embodiments, inhibiting SAMHD1 comprises contacting the SAMHD1 with a Vpx, BGLF4, M97, or KSHV ORF36 protein, or expressing the Vpx, BGLF4, M97, or KSHV ORF36 protein in a cell. In some embodiments, inhibiting SAMHD1 comprises contacting an mRNA encoding the SAMHD1 with a microRNA, shRNA, or siRNA that hybridizes to the mRNA, or expressing the microRNA, shRNA, or siRNA in a cell. In some embodiments, inhibiting SAMHD1 comprises contacting the SAMHD1 with a small molecule SAMHD1 inhibitor. In some embodiments, the enhancer is a DNA repair protein. In some embodiments, the DNA repair protein is a single-stranded endonuclease that removes flaps comprising a free 5′ end, i.e., flaps 3′ of a nick. In some embodiments, the DNA repair protein is a single-stranded endonuclease that removes flaps 5′ of a nick. In some embodiments, the DNA repair protein is flap structure-specific endonuclease 1 (FEN1) or a polynucleotide encoding FEN1. In some embodiments, the DNA repair protein is mutL homolog 1 (MLH1) or a polynucleotide encoding MLH1. In some embodiments, the enhancer comprises a dNTP synthetic enzyme. In some embodiments, the enhancer comprises a kinase. In some embodiments, the kinase comprises a nucleoside kinase, deoxynucleoside kinase, deoxynucleoside monophosphate kinase, or deoxynucleotide diphosphate. In some embodiments, the enhancer comprises Vpx, BGLF4, M97, KSHV1 ORF36, or FEN1. In some embodiments, the enhancer comprises Vpx and FEN1. In some embodiments, the enhancer comprises M97 and FEN1. In some embodiments, the FEN1 comprises a heterodimerization domain, e.g., a self-assembling coiled coil heterodimerization domain, for binding to a DNA-dependent DNA polymerase having the sequence of the other portion of the heterodimerization domain.

In some embodiments, the composition comprises a lipid nanoparticle composition comprising (a) a first polynucleotide encoding a SpyCas9 nickase and a DNA-dependent DNA polymerase; and (b) a second polynucleotide comprising, from 5′ to 3′, a spacer, a scaffold, a template sequence, and a DRS.

In some embodiments, the composition comprises a lipid nanoparticle composition comprising (a) a first polynucleotide encoding a SpyCas9 nickase and a DNA-dependent DNA polymerase; (b) a second polynucleotide comprising, from 5′ to 3′, a spacer and a scaffold; and (c) a third polynucleotide comprising, from 5′ to 3′, a template sequence and a DRS, wherein the second and third polynucleotide are provided as separate molecules, optionally wherein the third polynucleotide comprises an affinity tag, optionally an aptamer, for binding to the DNA-dependent DNA polymerase.

In some embodiments, the composition comprises a lipid nanoparticle composition comprising (a) a first polynucleotide encoding a SpyCas9 nickase; (b) a second polynucleotide encoding a DNA-dependent DNA polymerase; and (c) a third polynucleotide comprising, from 5′ to 3′, a spacer, a scaffold, a template sequence, and a DRS, wherein the first and second polynucleotide are provided as separate molecules, optionally wherein the third polynucleotide comprises an affinity tag, optionally an aptamer, for binding to the DNA-dependent DNA polymerase.

In some embodiments, the composition comprises a lipid nanoparticle composition comprising (a) a first polynucleotide encoding a SpyCas9 nickase; (b) a second polynucleotide encoding a DNA-dependent DNA polymerase; (c) a third polynucleotide comprising, from 5′ to 3′, a spacer and a scaffold; and (d) a fourth polynucleotide comprising, from 5′ to 3′, a template sequence and a DRS, wherein the first, second, third, and fourth polynucleotides are provided as separate molecules, optionally wherein the fourth polynucleotide comprises an affinity tag, optionally an aptamer, for binding to the DNA-dependent DNA polymerase.

In some embodiments, use of the composition results in template-based genome editing. In some embodiments, template-based genome editing results in edits comprising an insertion or deletion in a nucleotide sequence. In some embodiments, template-based genome editing results in edits comprising a substitution in a nucleotide sequence. In some embodiments, template-based genome editing results in at least 5%, 10%, 15%, 20%, 25%, 30%, 35%, 40%, 45%, optionally at least 50%, 55%, 60%, 65%, optionally at least 70%, 75%, or 80%, optionally at least 85%, 90%, 95%, 99% or more templated edits as a percent of total edits in the target nucleic acid. It is understood that non-templated edits, may occur during template-based genome editing, either alone or in conjunction with a templated edit. Non-templated edits include byproduct edits (i.e., mismatches or indels at or near the target site of the tgRNA or the nicking guide RNA, within the sequence analyzed by NGS, that are not encoded in the template). In determining the percent of template-based edits, the number of sequence reads with template-based edits, including byproduct edits, is determined as a percent of the total number of sequence reads. The number of template-based edits without byproduct edits, which are sometimes referred to herein as “perfect edits” or “templated edits” can be similarly determined. The percent of “perfect edits” or “templated edits” as a percent of total template-based edits can also be readily determined.

In some embodiments, use of the composition results in template-based genome editing in a cell or population of cells. In some embodiments, the cell is in vitro. In some embodiments, the cell is ex vivo. In some embodiments, the cell is in vivo. In some embodiments, the cell is a quiescent cell. In some embodiments, the cell is a quiescent cell selected from a hepatocyte, an inactivated T-cell, a muscle cell, a neuronal cell, and a lung cell. In some embodiments, the cell is a typically non-dividing cell type, i.e., a cell type that in adults does not typically divide in the absence of injury or tissue damage, e.g., liver cell, nerve cell, muscle cell. In some embodiments, the cell is a liver cell, such as a hepatocyte. In some embodiments, the cell is an immune cell such as a T cell. In some embodiments, at least 5% of the cells in the cell population comprise a template-based edit in the target nucleic acid. In some embodiments, at least 50% of the cells in the cell population comprise a desired genome edit in the target nucleic acid. In some embodiments, the cell is in vivo and at least 5% of the cells in the cell population comprise a desired genome edit in the target nucleic acid. In some embodiments, the cell is a typically non-dividing cell type in vivo and at least 5% of the cells in the cell population comprise a desired genome edit in the target nucleic acid. In some embodiments, the cell is a hepatocyte in vivo and at least 5% of the cells in the cell population comprise a desired genome edit in the target nucleic acid. In some embodiments, the cell is an immune cell and at least 50% of the cells in the cell population comprise a desired genome edit in the target nucleic acid. In some embodiments, the cell is a T cell and at least 50% of the cells in the cell population comprise a desired genome edit in the target nucleic acid.

In some embodiments, a method of producing a cell or a population of cells is provided. In some embodiments, the method comprises an insertion, deletion, or substitution in the genome of the target cell or cells, the method comprising contacting the cell or cells with (a) a SpyCas9 nickase or a nucleic acid encoding the same, (b) a DNA-dependent DNA polymerase or a nucleic acid encoding the same, and (c) a template guide RNA for template-based genome editing.

In some embodiments, the present disclosure provides, e.g., a system comprising a DNA-dependent DNA polymerase, a SpyCas9 nickase (nCas9), and a template guide RNA (tgRNA), wherein the tgRNA comprises a first polynucleotide and a second polynucleotide, wherein: (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; and (ii) a DNA-dependent DNA polymerase recruiting sequence (DRS) that is 6-20 nucleotides in length, optionally 6-17 or 10-14 nucleotides in length, and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid, wherein the DRS comprises DNA nucleotides; and wherein the DRS comprises at least one of: (1) a sequence not fully complementary to the DRS-complementary region of the non-target strand of the DNA duplex target nucleic acid; (2) a 1-5 nucleotide, optionally a 1-3 nucleotide 3′ terminal tail comprising a 3′ terminal modified uridine nucleotide; or (3) a 5′ terminal (D1) DNA nucleotide and a penultimate 5′ terminal (D2) RNA nucleotide wherein the 3′ end of the first polynucleotide is covalently linked to the 5′ end of the second polynucleotide by a phosphorothioate or phosphodiester linkage.

In some embodiments, the present disclosure provides, e.g., a system comprising a DNA-dependent DNA polymerase, a SpyCas9 nickase (nCas9), and a template guide RNA (tgRNA), wherein the tgRNA comprises a first polynucleotide and a second polynucleotide, wherein: (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; and (ii) a DNA-dependent DNA polymerase recruiting sequence (DRS) that is 6-20 nucleotides in length, optionally 6-17 or 10-14 nucleotides in length, and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid, wherein the DRS comprises RNA nucleotides; and wherein the DRS comprises at least one of: (1) a sequence not fully complementary to the DRS-complementary region of the non-target strand of the DNA duplex target nucleic acid; (2) a 1-5 nucleotide, optionally a 1-3 nucleotide 3′ terminal tail comprising a 3′ terminal modified uridine nucleotide; or (3) a 5′ terminal (D1) DNA nucleotide and a penultimate 5′ terminal (D2) RNA nucleotide wherein the 3′ end of the first polynucleotide is covalently linked to the 5′ end of the second polynucleotide by a phosphorothioate or phosphodiester linkage.

In some embodiments, the template sequence is at least 10-10,000 nucleotides in length. In some embodiments, the template sequence is at least 10, 1000, 2000, 3000, 4000, 5000, 6000, 7000, 8000, 9000, or 10,000 nucleotides in length. In some embodiments, the template sequence is at least 10-1000, 1000-2000, 2000-3000, 3000-4000, 4000-5000, 5000-6000, 6000-7000, 7000-8000, 8000-9000, or 9000-10,000 nucleotides in length. In some embodiments, the template sequence is at least 10-10,000, 1000-10,000, 2000-10,000, 3000-10,000, 4000-10,000, 5000-10,000, 6000-10,000, 7000-10,000, 8-10,000, or 9-10,000 nucleotides in length. In some embodiments, the template sequence is at least 10-1000, 10-2000, 10-3000, 10-4000, 10-5000, 10-6000, 10-7000, 10-8000, 10-9000, or 10-10,000 nucleotides in length.

In some embodiments, the template sequence is about 10-10,000 nucleotides in length. In some embodiments, the template sequence is about 10, 1000, 2000, 3000, 4000, 5000, 6000, 7000, 8000, 9000, or 10,000 nucleotides in length. In some embodiments, the template sequence is about 10-1000, 1000-2000, 2000-3000, 3000-4000, 4000-5000, 5000-6000, 6000-7000, 7000-8000, 8000-9000, or 9000-10,000 nucleotides in length. In some embodiments, the template sequence is about 10-10,000, 1000-10,000, 2000-10,000, 3000-10,000, 4000-10,000, 5000-10,000, 6000-10,000, 7000-10,000, 8-10,000, or 9-10,000 nucleotides in length. In some embodiments, the template sequence is about 10-1000, 10-2000, 10-3000, 10-4000, 10-5000, 10-6000, 10-7000, 10-8000, 10-9000, or 10-10,000 nucleotides in length.

In some embodiments, the template sequence comprises DNA nucleotides, wherein the plurality of nucleotides in the template sequence are DNA nucleotides. In some embodiments, 1-3 nucleotides at the 3′ end of the template (T1-T3), optionally only 1 RNA nucleotide at the 3′ end of the template (T1) are RNA nucleotides. In some embodiments, the template sequence consists of DNA nucleotides.

In some embodiments, the DRS comprises 6-20 nucleotides in length, the DRS is 6-17, 8-17, 10-12, 10-14, 10-16, or 12-14 nucleotides in length. In some embodiments, wherein the DRS comprises 6-17 nucleotides in length, the DRS is about 6-17, 8-17, 10-12, 10-14, 10-16, or 12-14 nucleotides in length. In some embodiments, the DRS is 6, 7, 8, 9, 10, 11, 12, 13, 14, 15, 16, 17, 18, 19, or 20 nucleotides in length.

In some embodiments, the DRS is 75-100% complementary to the DRS-complementary region of the non-target strand of the DNA duplex target nucleic acid. In some embodiments, the DRS is 100% complementary to the DRS-complementary region of the non-target strand of the DNA duplex target nucleic acid. In some embodiments, the DRS is fully complementary to the DRS-complementary region of the non-target strand of the DNA duplex target nucleic acid. In some embodiments, the DRS is not fully complementary to the DRS-complementary region of the non-target strand of the DNA duplex target nucleic acid. In some embodiments, the DRS has 0, 1, 2, 3, or 4 mismatches to the DRS-complementary region of the non-target strand of the DNA duplex target nucleic acid. In some embodiments, the DRS comprises 0, 1, 2, 3, or 4 mismatches to the DRS-complementary region of the non-target strand of the DNA duplex target nucleic acid. In some embodiments, the first 50% of nucleotides at the 5′ end of the DRS have 0 mismatches to the corresponding genomic sequence. In some embodiments, the first 50% of nucleotides at the 5′ end of the DRS have 1, 2, or 3 mismatches to the corresponding genomic sequence. In some embodiments, the first four nucleotides at the 5′ end of the DRS have 0, 1, 2, or 3 mismatches to the corresponding genomic sequence. In some embodiments, the first four nucleotides at the 5′ end of the DRS have 0 mismatches to the corresponding genomic sequence. In some embodiments, the first four nucleotides at the 5′ end of the DRS have 1 or 2 mismatches to the corresponding genomic sequence. In some embodiments, the first three nucleotides at the 5′ end of the DRS have 0 mismatches to the corresponding genomic sequence. In some embodiments, the first three nucleotides at the 5′ end of the DRS have 1 mismatch to the corresponding genomic sequence. In some embodiments, the first 6 nucleotides at the 5′ end of the DRS have 0, 1, or 2 mismatches to the corresponding genomic sequence. In some embodiments, the first 10 nucleotides at the 5′ end of the DRS have 0, 1, or 2 mismatches to the corresponding genomic sequence. In some embodiments, 70%-100% of the nucleotides in the first 10 nucleotides at the 5′ end of the DRS are complementary to the corresponding genomic sequence. In some embodiments, the DRS comprises, e.g., further comprises, one or more mismatches in the 3′ half of the DRS.

In some embodiments, the DRS comprises a 1-5 nucleotide 3′ terminal tail comprising a 3′ terminal modified uridine nucleotide. In some embodiments, the DRS comprises a 1-3 nucleotide 3′ terminal tail comprising a 3′ terminal modified uridine nucleotide. In some embodiments, the 3′ terminal tail is 1, 2, or 3 nucleotides in length.

In some embodiments, wherein the 3′ end of the first polynucleotide is covalently linked to the 5′ end of the second polynucleotide by a phosphorothioate or phosphodiester linkage, the DRS comprises a 5′ terminal (D1) DNA nucleotide and a penultimate 5′ terminal (D2) RNA nucleotide. In some embodiments, the 5′ terminal nucleotide in the DRS (D1) is a DNA nucleotide. In some embodiments, the penultimate 5′ nucleotide (D2) in the DRS is an RNA nucleotide. In some embodiments, each nucleotide from the penultimate 5′ nucleotide (D2) to the 3′ end nucleotide in the DRS is an RNA nucleotide.

In some embodiments, the first polynucleotide and the second polynucleotide are covalently linked. In some embodiments, the first polynucleotide and the second polynucleotide are not covalently linked. In some embodiments, the template guide RNA further comprises an affinity tag, wherein the first polynucleotide or the second polynucleotide are bound to a peptide or a peptide complex comprising a SpyCas9 nickase and a DNA-dependent DNA polymerase by the affinity tag.

In some embodiments, the method further comprises contacting the cell with a SpyCas9 nickase. In some embodiments, the scaffold of the SpyCas9 guide RNA binds to a SpyCas9 nickase. In some embodiments, the SpyCas9 nickase generates a nick on the same strand of a DNA duplex target nucleic acid as a nickase comprising an H840A mutation, i.e., the SpyCas9 is a nickase that cleaves the strand opposite to the strand that hybridizes to the spacer (i.e., the nickase cleaves the non-target strand of the DNA duplex target nucleic acid). In some embodiments, the SpyCas9 nickase generates a nick on the non-target strand of the DNA duplex target nucleic acid. In some embodiments, the SpyCas9 nickase comprises a catalytically inactive HNH domain. In some embodiments, the SpyCas9 nickase comprises a D839K, D839Q, D839N, D839A, H840S, H840T, H840Q, H840D, H840R, H840K, H840Y, H840N, N863S, N863T, N863A, N863E, or K866A mutation within its HNH-like nuclease domain. In some embodiments, the SpyCas9 nickase comprises an H840A point mutation.

In some embodiments, the method further comprises contacting the cell with a DNA-dependent DNA polymerase. In some embodiments, the DRS bound to the non-target strand of the DNA duplex target nucleic acid forms a binding site for a DNA-dependent DNA polymerase. In some embodiments, the DNA-dependent DNA polymerase comprises a eukaryotic DNA-dependent DNA polymerase. In some embodiments, the DNA-dependent DNA polymerase comprises a mammalian DNA-dependent DNA polymerase. In some embodiments, the DNA-dependent DNA polymerase comprises a human DNA-dependent DNA polymerase. In some embodiments, the DNA-dependent DNA polymerase comprises a cynomolgus DNA-dependent DNA polymerase. In some embodiments, the DNA-dependent DNA polymerase comprises a mouse DNA-dependent DNA polymerase. In some embodiments, the DNA-dependent DNA polymerase comprises a bacterial DNA-dependent DNA polymerase. In some embodiments, the DNA-dependent DNA polymerase comprises an E. coli DNA polymerase. In some embodiments, the DNA-dependent DNA polymerase comprises a viral DNA-dependent DNA polymerase. In some embodiments, the DNA-dependent DNA polymerase comprises a phage DNA-dependent DNA polymerase. In some embodiments, the DNA-dependent DNA polymerase comprises human DNA polymerase K, cynomolgus DNA polymerase K, mouse DNA polymerase K, human DNA polymerase θ, human DNA polymerase N, E. coli DNA polymerase I, phage T5 DNA polymerase, or phage Phi29 DNA polymerase. In some embodiments, the DNA-dependent DNA polymerase comprises human DNA polymerase K. In some embodiments, the DNA-dependent DNA polymerase comprises phage T5 DNA polymerase. In some embodiments, the DNA-dependent DNA polymerase does not comprise phage Phi29 DNA polymerase. In some embodiments, the DNA-dependent DNA polymerase comprises a point mutation(s) or deletion(s). In some embodiments, the DNA-dependent DNA polymerase comprises a point mutation(s) or deletion(s) within an exonuclease domain. In some embodiments, the one or more point mutation(s) or deletion(s) within the exonuclease domain abolishes exonuclease activity.

In some embodiments, the SpyCas9 nickase is operably linked to the DNA-dependent DNA polymerase. In some embodiments, the SpyCas9 nickase and DNA-dependent DNA polymerase comprise a single polypeptide. In some embodiments, the SpyCas9 nickase and DNA-dependent DNA polymerase comprise a single polypeptide expressed from a single open reading frame (ORF). In some embodiments, the SpyCas9 nickase and DNA-dependent DNA polymerase comprise a single polypeptide expressed from separate open reading frames (ORFs). In some embodiments, wherein the SpyCas9 nickase and DNA-dependent DNA polymerase comprise a single polypeptide expressed form separate open reading frames (ORFs). In some embodiments, the ORF encoding the SpyCas9 nickase further encodes a first intein and the ORF encoding the DNA-dependent DNA polymerase further encodes a second intein capable of binding the first intein (or capable of participating in a trans-splicing reaction with the first intein). In some embodiments, a first ORF encodes an N-terminal portion of the SpyCas9 nickase further encodes a first intein and a second ORF encodes a second intein the remainder of the SpyCas9 nickase (i.e., a C-terminal portion of SpyCas9) and a polymerase. In some embodiments, the SpyCas9 nickase and DNA-dependent DNA polymerase are provided as separate polypeptides. In some embodiments, the SpyCas9 nickase and DNA-dependent DNA polymerase directly interact through non-covalent interactions. In some embodiments, the SpyCas9 nickase and DNA-dependent DNA polymerase do not directly interact. As a first example, in some embodiments, the SpyCas9 nickase and DNA-dependent DNA polymerase may both non-covalently bind a single polynucleotide such as a template guide RNA. As a second example, in some embodiments, the SpyCas9 nickase may bind a first polynucleotide comprising a guide RNA and the DNA-dependent DNA polymerase may bind a second polynucleotide comprising a separable 3′ extension, wherein the first and second polynucleotides target the SpyCas9 nickase and DNA-dependent DNA polymerase to proximal genomic loci.

In some embodiments, the method comprises contacting a cell with a composition comprising (a) a single polypeptide comprising a SpyCas9 nickase and a DNA-dependent DNA polymerase; and (b) a single polynucleotide comprising, from 5′ to 3′, (i) a SpyCas9 guide RNA comprising a spacer and a scaffold; and (ii) a template sequence and a DRS.

In some embodiments, the method comprises contacting a cell with a composition comprising (a) a single polypeptide comprising a SpyCas9 nickase and a DNA-dependent DNA polymerase; (b) a first polynucleotide comprising a SpyCas9 guide RNA comprising, from 5′ to 3′, a spacer and a scaffold; and (c) a second polynucleotide comprising, from 5′ to 3′, a template sequence and a DRS, wherein the first and second polynucleotide are provided as separate molecules, optionally wherein the second polynucleotide comprises an affinity tag, optionally an aptamer, for binding to the DNA-dependent DNA polymerase.

In some embodiments, the method comprises contacting a cell with a composition comprising (a) a first polypeptide comprising a SpyCas9 nickase; (b) a second polypeptide comprising a DNA-dependent DNA polymerase; and (c) a single polynucleotide comprising, from 5′ to 3′, (i) a SpyCas9 guide RNA comprising a spacer and a scaffold; and (ii) a template sequence and a DRS, wherein the first and second polypeptide are provided as separate molecules, optionally wherein the second polynucleotide comprises an affinity tag, optionally an aptamer, for binding to a 3′ extension-recruiting domain on the DNA-dependent DNA polymerase. In some embodiments, the SpyCas9 nickase and DNA-dependent DNA polymerase directly interact through non-covalent interactions. In some embodiments, the SpyCas9 nickase and DNA-dependent DNA polymerase do not directly interact. In some embodiments, the SpyCas9 nickase and DNA-dependent DNA polymerase may both non-covalently bind a single polynucleotide such as a template guide RNA.

In some embodiments, the method comprises contacting a cell with a composition comprising (a) a first polypeptide comprising a SpyCas9 nickase; (b) a second polypeptide comprising a DNA-dependent DNA polymerase and a 3′ extension-recruiting domain; (c) a first polynucleotide comprising a SpyCas9 guide RNA comprising, from 5′ to 3′, a spacer and a scaffold; and (d) a second polynucleotide comprising, from 5′ to 3′, a template sequence and a DRS, and further comprising an affinity tag; wherein the first and second polypeptide and first and second polynucleotide are provided as separate molecules. In some embodiments, the SpyCas9 nickase and DNA-dependent DNA polymerase directly interact through non-covalent interactions. In some embodiments, the SpyCas9 nickase and DNA-dependent DNA polymerase do not directly interact. In some embodiments, the SpyCas9 nickase may bind a first polynucleotide comprising a guide RNA and the DNA-dependent DNA polymerase may bind a second polynucleotide comprising a separable 3′ extension, wherein the first and second polynucleotides target the SpyCas9 nickase and DNA-dependent DNA polymerase to proximal genomic loci.

In some embodiments, the method further comprises contacting the cell with a nicking guide RNA (ngRNA). The ngRNA comprises a spacer that hybridizes to a sequence such that the nick site of the ngRNA is within 200 nucleotides 5′ or 3′ from the nick site of the template guide RNA and on the target strand of the DNA duplex target nucleic acid. In some embodiments, the nick site of the ngRNA is about 20-200 nucleotides 5′ or 3′ from the nick site of the template guide RNA and on the target strand of the DNA duplex target nucleic acid. In some embodiments, the nick site of the ngRNA is within the genomic locus complementary to the spacer sequence of the template guide RNA. In some embodiments, the nick site of the ngRNA is preferably outside of the genomic locus complementary to the spacer sequence of the template guide RNA.

In some embodiments, the method further comprises contacting the cell with an enhancer of template-based genome editing. In some embodiments, the enhancer is an inhibitor of deoxynucleotide triphosphohydrolase. In some embodiments, wherein the enhancer is an inhibitor of deoxynucleotide triphosphohydrolase, the deoxynucleotide triphosphohydrolase comprises SAM domain and HD domain-containing protein 1 (SAMHD1). In some embodiments, inhibiting SAMHD1 comprises contacting the SAMHD1 with a Vpx, BGLF4, M97, or KSHV ORF36 protein or expressing the Vpx, BGLF4, M97, or KSHV ORF36 protein in a cell. In some embodiments, inhibiting SAMHD1 comprises contacting an mRNA encoding the SAMHD1 with a microRNA, shRNA, or siRNA that hybridizes to the mRNA, or expressing the microRNA, shRNA, or siRNA in a cell. In some embodiments, inhibiting SAMHD1 comprises contacting the SAMHD1 with a small molecule SAMHD1 inhibitor. In some embodiments, the enhancer is a DNA repair protein. In some embodiments, the DNA repair protein is a single-stranded endonuclease that removes flaps 5′ of a nick. In some embodiments, the DNA repair protein is flap structure-specific endonuclease 1 (FEN1) or a polynucleotide encoding FEN1. In some embodiments, the DNA repair protein is mutL homolog 1 (MLH1) or a polynucleotide encoding MLH1. In some embodiments, the enhancer comprises a dNTP synthetic enzyme. In some embodiments, the enhancer comprises a kinase. In some embodiments, the kinase comprises a nucleoside kinase, deoxynucleoside kinase, deoxynucleoside monophosphate kinase, or deoxynucleotide diphosphate. In some embodiments, the enhancer comprises Vpx, BGLF4, M97, KSHV ORF36, FEN1, or MLH1. In some embodiments, the enhancer comprises Vpx and FEN1. In some embodiments, the enhancer comprises M97 and FEN1. In some embodiments, the FEN1 comprises a heterodimerization domain, e.g., a self-assembling coiled coil heterodimerization domain, for binding to a DNA-dependent DNA polymerase having the sequence of the other portion of the heterodimerization domain.

In some embodiments, the method comprises contacting the cell with a composition comprising a lipid nanoparticle composition comprising (a) a first polynucleotide encoding a SpyCas9 nickase and a DNA-dependent DNA polymerase; and (b) a second polynucleotide comprising, from 5′ to 3′, a spacer, a scaffold, a template sequence, and a DRS.

In some embodiments, the method comprises contacting the cell with a composition comprising (a) a first polynucleotide encoding a SpyCas9 nickase and a DNA-dependent DNA polymerase; (b) a second polynucleotide comprising, from 5′ to 3′, a spacer and a scaffold; and (c) a third polynucleotide comprising, from 5′ to 3′, a template sequence and a DRS, wherein the second and third polynucleotide are provided as separate molecules, optionally wherein the third polynucleotide comprises an affinity tag, optionally an aptamer, for binding to the DNA-dependent DNA polymerase.

In some embodiments, the method comprises contacting the cell with a composition comprising a lipid nanoparticle composition comprising (a) a first polynucleotide encoding a SpyCas9 nickase; (b) a second polynucleotide encoding a DNA-dependent DNA polymerase; and (c) a third polynucleotide comprising, from 5′ to 3′, a spacer, a scaffold, a template sequence, and a DRS, wherein the first and second polynucleotide are provided as separate molecules, optionally wherein the third polynucleotide comprises an affinity tag, optionally an aptamer, for binding to the DNA-dependent DNA polymerase.

In some embodiments, the method comprises contacting the cell with a composition comprising a lipid nanoparticle composition comprising (a) a first polynucleotide encoding a SpyCas9 nickase; (b) a second polynucleotide encoding a DNA-dependent DNA polymerase; (c) a third polynucleotide comprising, from 5′ to 3′, a spacer and a scaffold; and (d) a fourth polynucleotide comprising, from 5′ to 3′, a template sequence and a DRS, wherein the first, second, third, and fourth polynucleotides are provided as separate molecules, optionally wherein the fourth polynucleotide comprises an affinity tag, optionally an aptamer, for binding to the DNA-dependent DNA polymerase.

In some embodiments, the method further comprises contacting the cell with a composition comprising a lipid nanoparticle comprising a polynucleotide encoding an enhancer of template-based genome editing. In certain embodiments, the polynucleotide encodes an shRNA for inhibiting expression of SAMHD1. In certain embodiments, the enhancer comprises Vpx. In certain embodiments, the enhancer comprises BGLF4. In certain embodiments, the enhancer comprises M97. In certain embodiments, the enhancer comprises KSHV ORF36. In certain embodiments, the enhancer comprises FEN1. In certain embodiments, the enhancer comprises mutL homolog 1 (MLH1).

In certain embodiments, contacting the cell with an enhancer comprises contacting the cell with Vpx and FEN1. In certain embodiments, Vpx and FEN1 are encoded on separate polynucleotides. In certain embodiments, Vpx and FEN1 are encoded on the same polynucleotide. In certain embodiments, Vpx and FEN1 are encoded on the same polynucleotide, e.g., on the same polynucleotide as a fusion protein. In some embodiments, wherein Vpx and FEN1 are encoded on the same polynucleotide as a fusion protein, the fusion protein comprises a peptide cleavage site between Vpx and FEN1.

In certain embodiments, contacting the cell with an enhancer comprises contacting the cell with M97 and FEN1. In certain embodiments, M97 and FEN1 are encoded on separate polynucleotides. In certain embodiments, M97 and FEN1 are encoded on the same polynucleotide. In certain embodiments, M97 and FEN1 are encoded on the same polynucleotide, e.g., on the same polynucleotide as a fusion protein.

In some embodiments, provided herein is a method of editing a cell, comprising contacting the cell with (a) a SpyCas9 nickase or a nucleic acid encoding the same, (b) a DNA-dependent DNA polymerase or a nucleic acid encoding the same, and (c) a template guide RNA for template-based genome editing, thereby inserting an exogenous DNA sequence near a cleavage site cleaved by the SpyCas9 nickase. In some embodiments, provided herein is a method of editing a cell, comprising contacting the cell with (a) a SpyCas9 nickase or a nucleic acid encoding the same, (b) a DNA-dependent DNA polymerase or a nucleic acid encoding the same, and (c) a template guide RNA for template-based genome editing, thereby deleting an endogenous DNA sequence near a cleavage site cleaved by the SpyCas9 nickase. In some embodiments, provided herein is a method of editing a cell, comprising contacting the cell with (a) a SpyCas9 nickase or a nucleic acid encoding the same, (b) a DNA-dependent DNA polymerase or a nucleic acid encoding the same, and (c) a template guide RNA for template-based genome editing, thereby replacing one or more nucleotides near a cleavage site cleaved by the SpyCas9 nickase.

In some embodiments, template-based genome editing results in edits comprising an insertion or deletion in a nucleotide sequence. In some embodiments, template-based genome editing results in edits comprising a substitution in a nucleotide sequence.

In some embodiments, template-based genome editing results in at least 5%, 10%, 15%, 20%, 25%, 30%, 35%, 40%, 45%, optionally at least 50%, 55%, 60%, 65%, optionally 70%, 75%, or 80% templated edits as a percent of total edits in the target nucleic acid (i.e., the number of NGS reads “templated edits” without byproduct edits divided by (the number of NGS reads with “templated edits” with or without byproduct edits plus the number of reads with indels without “templated edits”).

In some embodiments, use of the composition results in template-based genome editing in a cell or population of cells. In some embodiments, the cell is in vitro. In some embodiments, the cell is ex vivo. In some embodiments, the cell is in vivo. In some embodiments, the cell is a hepatocyte. In some embodiments, the cell is an immune cell such as a T cell. In some embodiments, at least 5% of the cells in the cell population comprise a desired genome edit in the target nucleic acid. In some embodiments, the cell is a hepatocyte and at least 5% of the cells in the cell population comprise a desired genome edit in the target nucleic acid. In some embodiments, the hepatocyte is in vivo. In some embodiments, the cell is an immune cell and at least 50% of the cells in the cell population comprise a desired genome edit in the target nucleic acid. In some embodiments, the cell is a T cell and at least 50% of the cells in the cell population comprise a desired genome edit in the target nucleic acid.

The present disclosure also relates to manufacturing methods to prepare cells in vitro or ex vivo for subsequent therapeutic administration to a subject. In some embodiments, the platform relates to genome editing via simultaneous or sequential administration of lipid nanoparticles (LNPs) comprising (a) a SpyCas9 nickase or a nucleic acid encoding the same, (b) a DNA-dependent DNA polymerase or a nucleic acid encoding the same, and (c) a template guide RNA for template-based genome editing. The systems and methods disclosed herein are relevant to any cell type but are particularly advantageous in preparing cells that require insertion, deletion, or substitution of a defined genomic sequence for full therapeutic applicability, e.g., in primary immune cells, e.g., T cells, B cells, NK cells; or stem cells including hematopoietic stem cells. As provided herein, the platform methods apply to “a cell” or to “a cell population” (or “population of cells”). When referring to delivery or gene editing methods for “a cell” herein, it is understood that the methods may be used for delivery or gene editing to “a cell population.”

In some embodiments, provided herein is use of any cell, population of cells, or composition disclosed herein for treating cancer or autoimmune disease. In some embodiments, provided herein is use of any cell, population of cells, or composition disclosed herein for preparation of a medicament for treating cancer or autoimmune disease. In some embodiments, provided herein is an engineered cell modified by the methods disclosed herein, and the engineered cell comprises at least one genomic modification, e.g., insertion, deletion, or substitution of nucleotides.

In some embodiments, provided herein is a cell treated in vitro with any method or composition disclosed herein. In some embodiments, provided herein is a cell treated in vivo with any method or composition disclosed herein. In some embodiments herein, a cell treated in vivo is a liver cell. In some embodiments herein, a cell treated in vivo is not a liver cell. In some embodiments, provided herein is a population of cells comprising any cell disclosed herein.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### Definitions

Unless stated otherwise, the following terms and phrases as used herein are intended to have the following meanings:

“Template-based genome editing”, “template-based editing”, “templated editing”, and the like, as used herein, is understood as a specific change to a nucleotide sequence, e.g., insertion, deletion, change in nucleotide sequence, dependent upon the sequence of an exogenous nucleotide template sequence and a polymerase delivered to the cell. As used herein, template-based editing uses an exogenously provided polynucleotide template and a DNA-templated DNA polymerase delivered to the cell. The nucleotide sequence of the template, and a complement thereof, is integrated into the genome (See, e.g., FIG. 1).

As used herein, a “transition” is a substitution mutation in which a purine nucleotide is replaced with a different purine nucleotide or a pyrimidine nucleotide is replaced with a different pyrimidine nucleotide. Exemplary transition mutations include A-to-G, G-to-A, C-to-T, and T-to-C.

As used herein, a “transversion” is a substitution mutation in which a purine nucleotide is replaced with a pyrimidine nucleotide or a pyrimidine nucleotide is replaced with a purine nucleotide. Exemplary transversion mutations include A-to-C, C-to-A, G-to-T, T-to-G, A-to-T, T-to-A, G-to-C, and C-to-G.

As used herein, “indel” refers to an insertion or deletion mutation consisting of a number of nucleotides that are either inserted, deleted, or inserted and deleted, e.g., at the site of a nick or double-strand break (DSB), in a target nucleic acid. As used herein, when indel formation results in an insertion, the insertion is a random insertion at the site of a nick or DSB and is not directed by or based on a template sequence.

As used herein, “base editing” is understood as a change in a nucleotide sequence promoted by a deamination reaction close to the site of a nick in a double stranded DNA molecule. That is, the change in the nucleotide sequence resulting from base editing is template independent and is not dependent on a polymerase. Deaminase reactions are catalyzed, for example, by a cytosine deaminase or an adenosine deaminase. The editing method is limited by both the nucleotide sequence present in the genome and the base changes that can be made through deamination, e.g., C to T and A to G. The compositions and methods provided herein do not include a deaminase.

As used herein, the term “operably linked” is understood as a juxtaposition of at least two components, e.g., polypeptide chain components or polynucleotide chain components, in a manner to allow one component to exert an effect on the other, or to allow the components when operably linked to have an effect that the two components could not exert individually or as a mixture of the components. In certain embodiments, two components may be operably linked by a covalent linkage, e.g., a peptide bond between two polypeptide chains encoded by a single open reading frame. In certain embodiments, the linkage can be mediated by a linker, e.g., a peptide linker. In certain embodiments, the linker is covalently attached to at least one of the components. In certain embodiments, the linker is covalently attached to both components. In certain embodiments, e.g., polynucleotides, the components can be joined by one or more phosphodiester or phosphorothioate bonds, e.g., a single PO or PS bond, or a polynucleotide linker sequence, or by other types of linkages, or any combination thereof.

In certain embodiments, components are joined by a linker not covalently attached to either component, e.g., two polypeptides bound to a single nucleic acid with binding sites for two distinct polypeptides, e.g., a guide RNA scaffold and an aptamer. In certain embodiments, polynucleotides, optionally polynucleotides bound to polypeptides, can be operably linked by being targeted to genomic loci with sufficient proximity to each other, typically on opposite strands of the DNA duplex target nucleic acid. For example, a nickase is recruited to a genomic locus by a first polynucleotide sequence comprising a spacer sequence 5′ of a guide scaffold sequence wherein the spacer sequence is complementary to the target strand of the DNA duplex target nucleic acid. Similarly, a polymerase is recruited to the non-target strand (opposite strand) of the DNA duplex target nucleic acid by a second polynucleotide sequence comprising a DNA polymerase recruiting sequence (DRS) complementary to the non-target strand of the DNA duplex target nucleic acid and a sequence to mediate binding of the second polynucleotide to the polymerase, e.g., an aptamer, 3′ of the DRS. The non-target strand of the DNA duplex target nucleic acid is available for hybridization to the DRS by the binding of the spacer sequence to the genomic locus and the nicking of the non-target strand by the nickase. The second polynucleotide further comprises a template sequence 5′ of the DRS.

In certain embodiments, components are joined directly by a non-covalent linkage. For example, each of the components can include a domain, e.g., a heterodimerization domain (or heterodimer domain, which is interchangeably used herein with a heterodimerization domain), that mediates the binding of the components to each other. In some embodiments, each of the components is a polypeptide chain and each polypeptide chain comprises a fusion protein comprising the heterodimer domain. In some embodiments, the heterodimer domains comprise leucine zipper domains, PDZ domains, streptavidin and streptavidin binding protein domains, foldon domains, hydrophobic polypeptides, an antibody or one or more binding fragments thereof (e.g., scFv, VHH domain) on one component that binds an epitope, either naturally occurring or inserted, in the other component. The position of the heterodimer domain is selected independently for each component. In some embodiments, the heterodimerization domain comprises a coiled coil heterodimerization domain, e.g., a self-assembling coiled coil heterodimerization domain. In some embodiments, the heterodimerization domain comprises an EBV, EI, KV, or KI domain, which may form a heterodimer with a corresponding self-assembling coiled coil heterodimerization domain. In some embodiments, the heterodimerization domain comprises an EI or KI domain. In some embodiments, the heterodimerization domain comprises an EI domain. In some embodiments, the heterodimerization domain comprises a KI domain. In some embodiments, the heterodimerization domain is SEQ ID NO: 1589, SEQ ID NO: 1593, or SEQ ID NO: 1595.

In certain embodiments, two polynucleotide chain components are operably linked by polypeptide binding, e.g., by two polynucleotide chains binding to a single polypeptide or two polynucleotide chains binding to a heterodimer.

As used herein, the term “system” refers to at least two operably linked elements, e.g., polypeptides or polynucleotides. In some embodiments, the system may comprise any two or more of a SpyCas9 nickase, a DNA-dependent DNA polymerase, a SpyCas9 guide RNA, and a 3′ extension. In some embodiments, the SpyCas9 nickase and DNA-dependent DNA polymerase may be covalently linked in a fusion protein. In some embodiments, the SpyCas9 nickase may be operably linked to a first split intein and the DNA-dependent DNA polymerase may be operably linked to a second split intein capable of participating in a trans-splicing reaction with the first intein. In some embodiments, the SpyCas9 nickase operably linked to the first intein and the DNA-dependent DNA polymerase operably linked to the second intein may be present as two separate polypeptides (pre-splicing). In some embodiments, the SpyCas9 nickase operably linked to the first intein and the DNA-dependent DNA polymerase operably linked to the second intein may be present as a single polypeptide (post-splicing). In some embodiments, the system may further comprise a nucleic acid encoding the SpyCas9 nickase or DNA-dependent DNA polymerase. In some embodiments, the SpyCas9 guide RNA may be covalently linked to the 3′ extension. In some embodiments, the SpyCas9 guide RNA may not be covalently linked to the 3′ extension. In some embodiments, the system may further comprise one or more enhancers of genomic editing, or one or more nucleic acids encoding the one or more enhancers of genomic editing.

As used herein, a “template guide”, “template guide RNA”, “tgRNA” or the like comprises a first polynucleotide sequence comprising a spacer sequence and a guide scaffold sequence, e.g., a guide RNA, wherein the spacer sequence is positioned 5′ of the guide scaffold sequence, and wherein the spacer sequence is targeted to a target strand of a DNA duplex target nucleic acid at a genomic locus (See, e.g., FIG. 2). The first polynucleotide sequence is operably linked to a second polynucleotide sequence, referred to herein as a 3′ extension, comprising a template sequence and a DNA-polymerase recruiting sequence (DRS), wherein the template sequence is positioned 5′ to the DRS, wherein the DRS is complementary, e.g., 75-100% complementary, to the non-target (i.e., opposite) strand of the double-stranded DNA molecule for at least six consecutive nucleotides within the spacer genomic target sequence, e.g., complementary over 6-20 consecutive nucleotides of the same genomic locus of the target locus of the spacer sequence. In certain embodiments, the DRS is less than 100% complementary to the non-target strand of the DNA duplex target nucleic acid. In certain embodiments, the DRS is 100% complementary to the genomic sequence across at least 3 base pairs, optionally at least 4 base pairs. In certain embodiments, the DRS has at least 6 base pairs with the genomic sequence across the length of the DRS. Mismatches between the DRS and the genomic sequence typically do not direct changes in the genomic sequence. Mismatches only at the 5′ end of the DRS can direct changes in the genomic sequence when the polymerase binds upstream of the mismatch(es) between the DRS and the genomic sequence such that the 5′ end of the DRS can act as a template. In certain embodiments, the DRS can direct a template based edit at one or more of positions D1, D2, and D3, optionally at position D1. In certain embodiments, the DRS position directing an edit is a DNA nucleotide. In certain embodiments, an edit directed by a nucleotide in the DRS is less efficient than an edit directed by the template. In certain embodiments, the positions that are less than 100% complementary to the non-target strand of the DNA duplex target nucleic acid that can direct template-based edits do not introduce changes into the amino acid sequence of the corresponding protein after template-based genome editing, i.e., silent mutations. It is understood that reducing the complementarity of the DRS to the non-target strand of the DNA duplex target nucleic acid will similarly reduce the complementarity of the DRS to the spacer sequence of the guide RNA. In certain embodiments, one or more mismatches between the DRS and the genomic target sequence are at the 5′ end of the DRS, e.g., within the first three nucleotides of the DRS.

In certain embodiments, the DRS further includes a 3′ tail that is not complementary to the genomic sequence, i.e., the mismatch is not flanked by nucleotides complementary to the genomic sequence. In certain embodiments, the 3′ tail of the DRS is 1-5 nucleotides in length, optionally 1, 2, or 3 nucleotides in length. In preferred embodiments, the most 3′ nucleotide in the 3′ tail is a uridine nucleotide. In certain embodiments, all of the nucleotides of the 3′ tail are uridine nucleotides. For the purpose of determining percent complementarity between the DRS and the genomic sequence, mismatches between the tail and the genomic sequence are not considered in percent identity.

The template sequence is less than 100% complementary to the non-target strand of the DNA duplex target nucleic acid. After polymerase-mediated extension, the nucleotide mismatch within the template sequence is incorporated into the genomic sequence such that the genomic sequence contains at least one template-based edit as compared to the unedited genomic sequence. The DRS and template sequence can further include mismatches to the guide RNA spacer sequence that do not result in changes in the corresponding amino acid sequence following template-based editing. Such mismatches may reduce the formation of DNA-RNA duplexes between the DRS and template sequence, and the guide RNA spacer sequence, that can be susceptible to cleavage by RNaseH. Such mismatches may reduce nuclease recruitment by the spacer sequence to genomic target sequence after template-based editing by changing the nucleotide sequence in the genomic locus to which the spacer sequence is targeted.

In certain embodiments, the first polynucleotide sequence, the guide RNA, and the second polynucleotide sequence, the 3′ extension, are operably linked by being in a single polynucleotide chain. In certain embodiments, the first polynucleotide sequence is 5′ of the second polynucleotide sequence in the polynucleotide chain. In certain embodiments, the first polynucleotide sequence is operably linked to the second polynucleotide sequence by a linker. In certain embodiments, the polynucleotide sequences are linked in the following orientation: 5′-first polynucleotide sequence-3′-linker-5′-second polynucleotide sequence-3′. In certain embodiments, the first polynucleotide sequence and the second polynucleotide sequence, optionally with the polynucleotide sequences bound to polypeptides, can be operably linked by being targeted to genomic loci with sufficient proximity to each other, typically on opposite strands of the DNA duplex target nucleic acid. In certain embodiments, the first polynucleotide sequence and the second polynucleotide sequence are in separate polynucleotide chains operably linked by being bound to a single polypeptide or a single polypeptide heterodimer. In certain embodiments, a first polynucleotide comprising the first polynucleotide sequence comprising the guide scaffold sequence is bound to a SpyCas9 enzyme portion of a polypeptide; and a second polynucleotide comprising the second polynucleotide sequence comprises an affinity tag polynucleotide sequence for binding to a corresponding polypeptide sequence in the polypeptide or polypeptide heterodimer. In certain embodiments, the affinity tag is 5′ to the template sequence. In certain embodiments, the affinity tag is 3′ to the DRS sequence. In certain embodiments, there is more than one affinity tag in the second polynucleotide. In certain embodiments, the affinity tag is operably linked to the template sequence or the DRS by a linker. In certain embodiments, the template sequence and affinity tag are linked in the following orientation: In certain embodiments, the template sequence and affinity tag are linked in one of the following orientations: 5′-affinity tag-template-DRS-3′; 5′-template-DRS-affinity tag; or 5′-affinity tag-template-DRS-affinity tag-3′. In certain embodiments, one or more linkers are present between the one or more of the DRS, affinity tag, and template. In certain embodiments, the corresponding polypeptide sequence for binding the affinity tag is present on a DNA dependent DNA polymerase or SpyCas9.

As used herein, “ligated template guide RNA,” “ligated tgRNA,” and the like are used herein to refer to a tgRNA produced by ligating at least two or at least three separate polynucleotides, e.g., two or three separate polynucleotides.

As fused herein, a “splint oligonucleotide” is an oligonucleotide that comprises a first region of complementarity to a first polynucleotide and a second, immediately adjacent region of complementarity to a second polynucleotide. The splint oligonucleotide is therefore capable of simultaneously annealing to the ends of two polynucleotides that flank a ligation site, thereby bringing them into proximity so that they may be ligated together (e.g., via a ligase).

“Guide RNA”, “gRNA”, and “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally occurring sequence, or a trRNA sequence with modifications or variations compared to naturally occurring sequences.

As used herein, a “nicking guide RNA” is a guide RNA, typically an sgRNA, that can bind to the same Cas9 enzyme, e.g., a SpyCas9 enzyme, as the template guide, but that does not contain a DRS or a template sequence. The target site for the spacer sequence in a nicking guide RNA is about 1-200 nucleotides, preferably about 1-100 nucleotides 5′ or 3′ from the nicking site for the template guide, on the opposite DNA strand in the genome i.e., the non-target strand of the DNA duplex target nucleic acid. In certain embodiments, the nicking guide RNA facilitates SpyCas9 nicking between 20-200 nucleotides 5′ or 3′ from the nicking site for the template guide on the opposite DNA strand of the DNA duplex target nucleic acid. In cases where the target site for the nicking guide RNA is within 20 nucleotides of the template guide RNA nicking site, the targeting sequence of the nicking guide RNA preferably already has the intended edits. In preferred embodiments, the nicking guide RNA does not promote editing at its target site.

“Internal linker” as used herein describes a non-nucleotide segment joining two nucleotides within a guide RNA. If the guide RNA contains a spacer region, the internal linker is located outside of the spacer region (e.g., in the scaffold or conserved region of the guide RNA). Exemplary structures of guide RNA scaffolds with internal linkers are provided in WO2022261292, the content of which is incorporated by reference. In some embodiments, an internal linker may join two nucleotides within a template guide RNA. For example, an internal linker may join the guide RNA and 3′ extension, the template sequence and an affinity tag, or the DRS and an affinity tag. In some cases, the non-nucleotide linker having the same structure as an internal linker may be also used to join two nucleotides elsewhere and may be referred to as a non-nucleotide linker.

As used herein, a “spacer,” “spacer sequence,” “guide sequence,” “guide region,” “targeting sequence” and the like refers to a sequence within a guide RNA that is complementary to a target sequence and functions to direct a guide RNA to a target sequence for binding or modification (e.g., cleavage) by an RNA-guided nickase. A guide sequence can be 20 nucleotides in length, e.g., in the case of Streptococcus pyogenes (i.e., SpyCas9 (also referred to as SpCas9)) and related Cas9 homologs/orthologs. Shorter or longer sequences can also be used as guides, e.g., 17-, 18-, 19-, or 21-nucleotides in length.

Exemplary “scaffold” or “conserved region” of a S. pyogenes Cas9 (“spyCas9” (also referred to as “spCas9”)) sgRNA” is shown in Tables 5A-5B. The first row shows the numbering of the nucleotides; the second row shows the sequence (e.g., SEQ ID NO: 22 or 115); and the third row shows the regions. Various SpyCas9 guide RNA scaffold sequences and chemical modifications are known in the art including, but not limited to, those provided in WO2018107028, WO2019237069, WO2021119275, and WO2022261292, the contents of each of which are herein incorporated by reference.

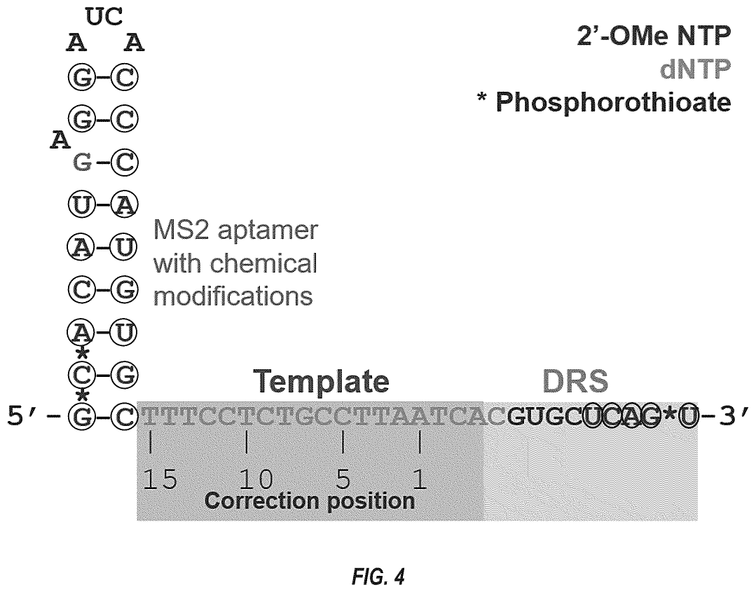

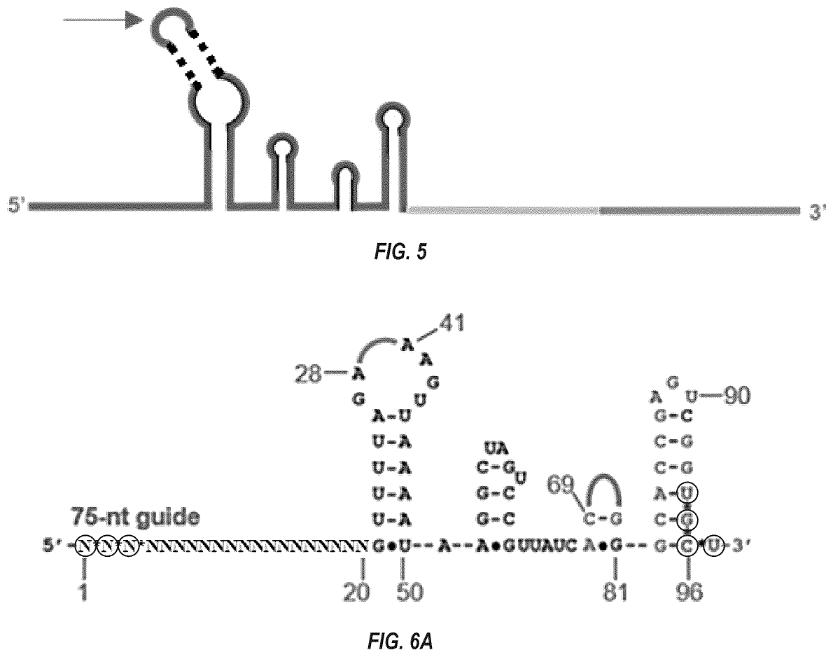

As used herein, a “3′ extension,” “3′ extension polynucleotide,” or the like is understood as a polynucleotide containing, at least a template sequence 5′ to a DNA-dependent DNA polymerase recruiting sequence (DRS), wherein the 3′ extension is operably linked to a spacer sequence and a guide scaffold sequence, e.g., a guide RNA (See, e.g., FIGS. 2-6I). In certain embodiments, the 3′ extension is covalently linked to the 3′ end of the guide scaffold sequence. In certain embodiments, the covalent linkage is a phosphodiester bond or a phosphorothioate bond, and can include a polynucleotide linker sequence. In certain embodiments, the covalent linkage is a non-nucleotide linkage. In certain embodiments, the linkage is a non-covalent linkage, e.g., an operable linkage as provided herein. As used herein, a 3′ extension that is not covalently linked to a guide scaffold sequence may be referred to as a separable 3′ extension.

In the context of the 3′ extension, the boundary between the template sequence and the DRS may be defined relative to the position of the nick generated by the SpyCas9 nickase. The nucleotide at the 5′ end of the flap 3′ of the nick is complementary to the nucleotide at the 3′ end of the template (unless an edit is to be introduced at position T1), while the nucleotide at the 3′ end of the flap 5′ of the nick is complementary to the nucleotide at the 5′ end of the DRS. Accordingly, the boundary between the template sequence and the DRS may be described relative to the extension activity of the DNA-dependent DNA polymerase, with the first nucleotide of the template (T1; the nucleotide immediately 5′ of the boundary between the template sequence and the DRS) being complementary to the first nucleotide of the newly synthesized DNA strand.

As used herein, the term “3′ extension-recruiting domain” (or “template-recruiting domain,” “aptamer-binding domain,” and the like) is understood as a polypeptide sequence or a polynucleotide sequence, a small molecule, or the like that binds to a cognate affinity tag covalently linked to a 3′ extension disclosed herein. In some embodiments, the 3′ extension-recruiting domain is at the C-terminal of the DNA-dependent DNA polymerase. In some embodiments, the 3′ extension-recruiting domain is at the N-terminal of the DNA-dependent DNA polymerase.

As used herein, a “chimeric DNA/RNA 3′ extension” or “chimeric DNA/RNA 3′ extension polynucleotide”, or “3′ extension”, is understood as a 3′ extension polynucleotide having a template portion with a sufficient number of DNA nucleotides (2′-H and 3′-OH) to permit extension by a DNA-dependent DNA polymerase when a DRS portion having independently at each position a DNA nucleotide, an RNA nucleotide or modified RNA nucleotide wherein the modification is tolerated in the scaffold of a guide RNA (e.g., 2′-OH, 2′-Ome, 2′F modified nucleotide) has formed a duplex region with the 3′ end of the flap formed by a nick in the genomic DNA (the flap 5′ of the nick). In certain embodiments, the template portion of the chimeric DNA/RNA 3′ extension includes all DNA nucleotides. In certain embodiments, the DRS portion of the chimeric DNA/RNA 3′ extension includes all DNA nucleotides. In certain embodiments, the 3′ end of the DRS includes all RNA nucleotides, e.g., modified RNA nucleotides, and the 5′ end of the DRS includes DNA nucleotides. In certain embodiments, 1-3 nucleotides on the 5′ end of the DRS (D1-D3) are DNA nucleotides. In certain embodiments, 0-7 nucleotides on the 5′ end of the DRS (D1-D7) are DNA nucleotides. In certain embodiments, the 3′ nucleotide of the DRS is an RNA nucleotide or modified RNA nucleotide and the remaining nucleotides are DNA nucleotides. In certain embodiments, the template portion of the chimeric DNA/RNA 3′ extension includes all DNA nucleotides and the 3′ end of the DRS includes all RNA or modified RNA nucleotides, and the 5′ end of the DRS includes DNA nucleotides. In certain embodiments, 1-3 nucleotides on the 3′ end of the DRS are modified nucleotides, e.g., 2′-Ome modified nucleotides. In certain embodiments, the 3′ end of the DRS has 1-3 phosphorothioate modifications, preferably with a single phosphorothioate, e.g., between the most 3′ nucleotides. In certain embodiments, 1-3 nucleotides on the 3′ end of the DRS are modified nucleotides, e.g., independently 2′-Ome and phosphorothioate modified nucleotides. In certain embodiments, the DRS has a 3′ tail. In certain embodiments, the 3′ tail of the DRS is 1-5 nucleotides in length, optionally 1, 2, or 3 nucleotides in length. In preferred embodiments, the most 3′ nucleotide in the 3′ tail is a uridine nucleotide. In certain embodiments, all of the nucleotides of the 3′ tail are uridine nucleotides. In certain embodiments, the most 3′ uridine nucleotide of the 3′ extension is a modified uridine nucleotide. In certain embodiments, all of the nucleotides of the 3′ tail are modified uridine nucleotides.

“Template sequence” as used herein is a nucleotide sequence 5′ to the DRS, wherein the template sequence provides a nucleotide sequence different from the nucleotide sequence in the strand of genomic DNA prior to editing, wherein the template directs incorporation of at least one nucleotide sequence change into the genome, i.e., template-based genome editing. Such incorporations can come in the form of nucleotide substitutions, insertions or deletions. In some embodiments, the template sequence is at least 5 nucleotides in length, preferably at least 10 nucleotides in length. In preferred embodiments, the template sequence is 100% complementary to the genomic sequence for at least 4 consecutive nucleotides prior to template-based genome editing. In certain embodiments, the 3′ portion of the template sequence includes the nucleotide sequence that is different from the nucleotide sequence in the strand of the genomic DNA prior to template-based genome editing. In certain embodiments, the most 3′ position of the template sequence is a nucleotide that is different from the nucleotide sequence of the strand of the DNA duplex target nucleic acid prior to template-based genome editing. In certain embodiments, the 5′ terminal portion of the template sequence is 100% complementary to the non-target strand of the DNA duplex target nucleic acid across four consecutive nucleotides. The template sequence preferably directs at least one change in the genomic sequence that alters the expression or sequence of an encoded polypeptide, e.g., a change in at least one encoded amino acid, or a change in a promoter or other regulatory element that alters expression of the sequence under control of the regulatory element. In certain embodiments, the positions of the template that are less than 100% complementary to the non-target strand of the DNA duplex target nucleic acid do not introduce changes into a protein sequence after template-based genome editing, i.e., silent mutations. In certain embodiments, the template sequence directs the incorporation of a change in the genomic sequence within the PAM, i.e., by altering the identity or position of one of the G nucleotides relative to the spacer binding site in the non-target strand. In certain embodiments, the incorporation of a change within the genomic sequence of the PAM is a silent mutation. In certain embodiments, the template sequence directs the incorporation of a change in the genomic sequence within the spacer sequence, e.g., within the seed sequence of the spacer, i.e., within the 3-7 nucleotides adjacent to the 5′ end of the PAM sequence in the non-target strand. In certain embodiments, the incorporation of a change within the genomic sequence of the spacer sequence is silent mutation. The introduction of silent mutations within the spacer or PAM sequence can be used to reduce nickase recruitment to the target site.

As used herein, “a DNA polymerase-recruiting sequence” or “DRS” is located 3′ of a template sequence in a 3′ extension. In some embodiments, the DRS can be about 6-20 nucleotides in length, e.g., 6-17, 8-17, 10-12, 10-14, 10-16, or 12-14 nucleotides in length. In certain embodiments, the DRS is complementary or partially complementary to the flap 5′ of the nick, e.g., contains no more than one mismatch within the first 6 nucleotides at the 5′ end of the DRS in the genomic DNA 5′ to the site for nicking by the nickase. However, it is understood that the length and sequence to which the DRS is complementary is determined, in part, by the nucleotide sequence of the genomic locus to be edited and the availability of a PAM to permit nicking of the genomic DNA. In certain embodiments, the DRS is less than 100% complementary to the non-target strand of the genome. It is understood that reducing the complementarity of the DRS to the non-target strand of the DNA duplex target nucleic acid similarly reduces the complementarity of the DRS to the spacer sequence of the guide RNA. In certain embodiments, the DRS is 100% complementary to the genomic sequence across at least 3 base pairs, optionally at least 4 base pairs. In certain embodiments, the DRS has at least 6 base pairs with the genomic sequence across the length of the DRS. In certain embodiments, the DRS comprises 1 or 2 mismatches within the 5′ half of the DRS, i.e., the end closer to the DRS-template sequence junction. In certain embodiments, the DRS comprises 1 or 2 mismatches within the four most 5′ nucleotides in the DRS. In certain embodiments, the DRS comprises, e.g., further comprises, one or more mismatches in the 3′ half of the DRS. In certain embodiments, the DRS further includes a 3′ tail that is not complementary to the genomic sequence, i.e., the mismatch is not flanked by nucleotides complementary to the genomic sequence. In certain embodiments, the 3′ tail of the DRS is 1-5 nucleotides in length, optionally 1, 2, or 3 nucleotides in length. In preferred embodiments, the most 3′ nucleotide in the 3′ tail is a uridine nucleotide. In certain embodiments, all of the nucleotides of the 3′ tail are uridine nucleotides. For the purpose of determining percent complementarity between the DRS and the genomic sequence, mismatches between the tail and the genomic sequence are not considered in percent identity. In some embodiments, one to three consecutive nucleotides from the 5′ end of the DRS is a DNA nucleotide (2′-H). In other embodiments, the DRS contains entirely DNA nucleotides. In some embodiments, the DRS contains a mixture of DNA and RNA nucleotides. In some embodiments, the 3′ end of the DRS can include non-DNA nucleotides, RNA nucleotides including modified RNA nucleotides, e.g., 2′-OH, 2′-Ome, 2′F, 3′-amine, abasic nucleotides, and 3′-PEG nucleotides; dideoxynucleotides and non-nucleotide linkers.

As used herein, the term “affinity tag” is understood to refer to a structural element that binds to a polynucleotide sequence, polypeptide sequence, small molecule, or the like. In certain embodiments, affinity tags may be used to mediate binding of polynucleotides such as guide RNAs to a corresponding polypeptide. In certain embodiments, an affinity tag may be an aptamer. In certain embodiments, an affinity tag may be a small molecule such as biotin, desthiobiotin, or digoxigenin.

As used herein, the term “aptamer” is understood to refer to a structural polynucleotide that binds to a polypeptide sequence, small molecule, or the like. In certain embodiments, aptamers may be used to mediate binding of polynucleotides such as guide RNAs to a corresponding polypeptide. Aptamers may be naturally occurring. For example, some viral nucleic acids bind viral proteins, e.g., the MS2 hairpin that binds to the bacteriophage MS2 coat protein (MCP). As an additional example, bacterial nucleic acids may bind portions of bacterial proteins, e.g., domains from CRISPR endonucleases that bind guide RNAs. Other aptamers may be designed or generated through molecular evolution using known methods in the art. As used herein, the term aptamer does not include the portion of a guide RNA scaffold that binds to a SpyCas9 enzyme.

As used herein, aptamers are single-stranded oligonucleotides that fold into well-defined three-dimensional shapes, allowing them to bind their targets, preferably with high affinity and specificity. Aptamers fold into defined three-dimensional shapes determined by their primary sequences that are usually about 10-80 nt long. Aptamer structures include binding pockets and clefts through which they bind their respective targets by a combination of van der Waals, hydrogen bonding, salt bridges, hydrophobic and electrostatic interactions for specific recognition. (see, e.g., Banerjee and Nilsen-Hamilton, 2013. Aptamers: multifunctional molecules for biomedical research. Journal of Molecular Medicine. 91:1333-1342.)

In certain embodiments, an aptamer may be the MS2 hairpin (alternatively referred to herein as a MS2-loop, MS2 aptamer, or MS2 RNA loop), which binds the bacteriophage MS2 coat protein (MCP), both of which are well known in the art. The coat protein dimer of bacteriophage MS2 binds specifically to an RNA hairpin in the viral genome, thereby repressing the translation of the viral replicase. This hairpin/translational operator (TR) corresponds to nucleotide positions-15 to +4 relative to the start of the replicase gene and is 19 nt in length. The wild-type TR consists of a 7 base pair stem, interrupted by one unpaired residue between base pairs five and six, and is closed by a 4-nt loop. Various MS2 hairpin and MCP sequences are provided, for example, in Helgstrand et al., 2002. Investigating the structural basis of purine specificity in the structures of MS2 coat protein RNA translational operator hairpins NAR 30:2678-2685; and Lim et al., 1994. Altering the RNA binding specificity of a translational repressor. 269:9006-9010. Thus, the structures and requirements for binding of the MS2 hairpin and MCP are well known in the art.

In certain embodiments, the aptamer may be a cognate RNA for binding the PP7 coat protein, which is well characterized. The structure of the cocrystal of the PP7 coat protein and its cognate RNA reveals the molecular basis of the interaction, and it demonstrates that the conserved beta-sheet surface is a flexible architecture that can evolve to recognize diverse RNA hairpins. Various sequences for the PP7 coat protein and cognate RNA and requirements for binding are known in the art. (see, e.g., Chao et al., 2008. Structural basis for the coevolution of a viral RNA-protein complex www.nature.com/doifinder/10.1038/nsmb1327).

In certain embodiments, aptamers and their cognate binding peptides can be derived from bacterial proteins, including portions of CRISPR proteins and nucleic acids, e.g., polypeptide sequences from CRIPSR endonucleases that bind to portions of cognate guide RNAs sequences. Structural studies have defined these interactions between CRISPR endonucleases and guide RNAs see, e.g., Haurwitz et al., Science. 2010; 329:1355-8. doi: 10.1126/science.1192272 and Mulepati et al., Science. 2014; 345:1479-84. doi: 10.1126/science.1256996. Based on these studies, as well as others, those of skill in the art could identify RNA sequences for use as aptamers with their cognate protein binding sequences.

“Polypeptide” as used herein refers to a multimeric compound comprising amino acid residues that can adopt a three-dimensional conformation. Polypeptides include but are not limited to enzymes, enzyme precursor proteins, regulatory proteins, structural proteins, receptors, nucleic acid binding proteins, antibodies, etc. Polypeptides may, but do not necessarily, comprise post-translational modifications, non-natural amino acids, prosthetic groups, and the like. In some embodiments, a polypeptide may comprise an amino acid sequence provided by a SEQ ID NO listed herein (e.g., in Table 6) that includes an N-terminal methionine. In some embodiments, such a polypeptide may comprise the SEQ ID NO comprising the N-terminal methionine. In other embodiments, the polypeptide may comprise the SEQ ID NO: as listed, but without the N-terminal methionine. For example, a SEQ ID NO provided herein may, in some embodiments, be operably linked to another moiety at the N-terminus (e.g., a nuclear localization signal, a second polypeptide, etc.). In such embodiments, a person of skill in the art will recognize that the N-terminal methionine is optional. Accordingly, in SEQ ID NOs provided herein that contain an N-terminal methionine residue, the N-terminal methionine residue should be considered an optional feature. Likewise, for nucleic acid sequences provided herein which encode a protein or polypeptide that comprises an N-terminal methionine residue, the nucleotides encoding the N-terminal methionine residue should be considered an optional feature.

As used herein, “Cas nuclease”, also called “Cas protein” as used herein, encompasses Cas cleavases, Cas nickases, and dCas DNA binding agents. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity. Class 2 Cas nucleases include Class 2 Cas cleavases, Class 2 Cas nickases (e.g., H840A, D10A, or N863A variants), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9 (1.0) (e.g., K810A, K1003A, R1060A variants), and eSPCas9 (1.1) (e.g., K848A, K1003A, R1060A variants) proteins and modifications thereof. Cpf1 protein, Zetsche et al., Cell, 163:1-13 (2015), is homologous to Cas9, and contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015). In certain embodiments, the Cas9 is a Streptococcus pyogenes Cas9 (SpyCas9)

As used herein, a “nickase” is an enzyme that creates a single-strand break (also known as a “nick”) in double strand DNA, i.e., cuts one strand but not the other of the DNA double helix. As used herein, an “RNA-guided nickase” means a polypeptide or complex of polypeptides having DNA nickase activity, wherein the DNA nickase activity is sequence-specific and depends on the sequence of the RNA. Exemplary RNA-guided nickases include Cas nickases particularly Spy Cas9 nickases. Cas nickases include, but are not limited to, nickase forms of a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. Class 2 Cas nickases include Class 2 Cas nuclease variants in which only one of the two catalytic domains is inactivated, which have RNA-guided DNA nickase activity. Class 2 Cas nickases include, for example, Cas9 (e.g., H840A, D10A, or N863A variants of SpyCas9), Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9 (1.0) (e.g., K810A, K1003A, R1060A variants), and eSPCas9 (1.1) (e.g., K848A, K1003A, R1060A variants) proteins and modifications thereof. Cpf1 protein, Zetsche et al., Cell, 163:1-13 (2015), is homologous to Cas9, and contains a RuvC-like protein domain. Cpf1 sequences of Zetsche are incorporated by reference in their entirety. See, e.g., Zetsche, Tables S1 and S3. “Cas9” encompasses S. pyogenes (Spy) Cas9, the variants of Cas9 listed herein, and equivalents thereof. See, e.g., Makarova et al., Nat Rev Microbiol, 13(11): 722-36 (2015); Shmakov et al., Molecular Cell, 60:385-397 (2015).

As used herein, a “DNA-dependent DNA polymerase” or “DNA-templated DNA polymerase” (DDP) synthesizes DNA opposite a nucleic acid template preferentially comprised of DNA over RNA. In some embodiments, the extension activity of a DDP on DNA templates consisting only of DNA nucleotides is at least 4-fold higher than the extension activity of the DDP on RNA templates consisting only of RNA nucleotides, as measured, for example using the method provided in Example 3 below. Editing is compared between a template guide having a fully RNA DRS and a fully RNA template against a template guide having a fully RNA DRS and a fully DNA template. Consequently, editing by the editors disclosed herein (a complex comprising a SpyCas9 nickase operably linked to a DDP using a template guide RNA) is at least 10-fold, optionally 20-fold higher than editing with tgRNAs containing RNA template sequences. In some embodiments, repair DDPs such as human DNA polymerase K may tolerate a template in which fewer than 100% of the nucleotides are DNA nucleotides. For example, in some embodiments, repair DDPs such as human DNA polymerase K may tolerate a template in which 1, 2, or 3 of the nucleotides are RNA nucleotides.

In certain embodiments, DNA-dependent DNA polymerases are prokaryotic DNA polymerases. In certain embodiments, DNA-dependent DNA polymerases are viral DNA polymerases. In certain embodiments, DNA-dependent DNA polymerases are eukaryotic DNA polymerases.

In certain embodiments, DNA-dependent DNA polymerases are wild-type DNA polymerases. In certain embodiments, DNA-dependent DNA polymerases are variant DNA polymerases, i.e., not wild-type DNA polymerases. In certain embodiments, a variant DNA polymerase has altered activity as compared to the wild-type polymerase. For example, in certain embodiments, the processivity of the variant polymerase may be increased as compared to the wild-type polymerase. In certain embodiments, an exonuclease or certain exonuclease activity of the variant polymerase may be decreased or abolished as compared to the wild-type polymerase.

As used herein, an “enhancer” or an “enhancer of template-based genome editing” is a nucleic acid, e.g., siRNA or shRNA, or protein for increasing the efficiency of template-based genome editing. In certain embodiments, one or more enhancers may be used to increase template-based genome editing. See, e.g., FIG. 3D, which shows exemplary mechanisms of action for different enhancers.

In certain embodiments, an enhancer of template-based genome editing is a DNA repair protein, e.g., an mRNA encoding a DNA repair protein or a DNA repair protein. In certain embodiments, the DNA repair protein is a single-strand endonuclease responsible for removing flaps generated at 3′ of a nick. In certain embodiments, removal of flaps 3′ of a nick includes expression of flap structure-specific endonuclease 1 (FEN1) protein (see, e.g., www.ncbi.nlm.nih.gov/gene/2237 which is incorporated herein by reference in the version available as of the earliest filing date of this application). The FEN1 protein removes 5′ overhanging flaps in DNA repair and processes the 5′ ends of Okazaki fragments in lagging strand DNA synthesis. Direct physical interaction between this protein and AP endonuclease 1 during long-patch base excision repair provides coordinated loading of the proteins onto the substrate, thus passing the substrate from one enzyme to another. In certain embodiments, the DNA repair protein is mutL homolog 1 (MLH1) (see, e.g., www.ncbi.nlm.nih.gov/gene/4292/, incorporated herein by reference in the version available on the earliest filing date of the instant application). The MLH1 protein can heterodimerize with mismatch repair endonuclease PMS2 to form MutL alpha, part of the DNA mismatch repair system. When MutL alpha is bound by MutS beta and some accessory proteins, the PMS2 subunit of MutL alpha introduces a single-strand break near DNA mismatches, providing an entry point for exonuclease degradation. In certain embodiments, such an enhancer is used in a dividing cell, e.g., a T cell, e.g., an activated T cell. In certain embodiments, such enhancers are used in a cell that is typically a non-dividing cell, e.g., a liver cell, a monocyte, a neuron, a hematopoietic stem cell, or a muscle cell.

In certain embodiments, an enhancer of template-based genome editing increases the availability of deoxynucleoside triphosphate (dNTP), e.g., the concentration of dNTP in the nucleus relative to a cell not contacted with such an enhancer, particularly in a cell that is typically a non-dividing cell, e.g., a liver cell. In certain embodiments, increasing the dNTP concentration in the cell comprises inhibiting a deoxynucleotide triphosphate triphosphohydrolase in the cell. In certain embodiments, the deoxynucleotide triphosphate triphosphohydrolase comprises SAM domain and HD domain-containing protein 1 (SAMHD1) (see, e.g., ncbi.nlm.nih.gov/gene/25939, which is incorporated herein by reference in the version available as of the earliest filing date of this application). In certain embodiments, inhibiting SAMHD1 comprises contacting the SAMHD1 with a Vpx protein, or expressing the Vpx protein in the cell (see, e.g., www.ncbi.nlm.nih.gov/gene/1724714, which is incorporated herein by reference in the version available as of the earliest filing date of this application). In various aspects, inhibiting SAMHD1 comprises contacting the SAMHD1 with a BGLF4 protein, or expressing the BGLF4 protein in the cell (see, e.g., www.ncbi.nlm.nih.gov/gene/3783704, which is incorporated herein by reference in the version available as of the earliest filing date of this application). In various aspects, inhibiting SAMHD1 comprises contacting the SAMHD1 with an M97 protein, or expressing the M97 protein in a cell (see, e.g., www.ncbi.nlm.nih.gov/gene/80532996, which is incorporated herein by reference in the version available as of the earliest filing date of this application). In various aspects, inhibiting SAMHD1 comprises contacting the SAMHD1 with an KSHV ORF36 protein, or expressing the KSHV ORF36 protein in a cell. In various aspects, inhibiting SAMHD1 comprises contacting an mRNA encoding the SAMHD1 with a microRNA, shRNA, or siRNA that hybridizes to the mRNA, or expressing the microRNA, shRNA or siRNA in the cell. In certain embodiments, inhibiting SAMHD1 comprises contacting the SAMHD1 with a small molecule SAMHD1 inhibitor. In certain embodiments, increasing the dNTP concentration in the cell comprises administering dNTPs to the cell. In certain embodiments, administering dNTPs to the cell comprises administering dNTPs to a subject comprising the cell. In various aspects, increasing the dNTP concentration in the cell comprises administering nucleosides or nucleotides to the cell. The nucleosides or nucleotides may include deoxynucleosides (dNs), deoxynucleoside monophosphates (dNMPs), or nucleoside triphosphates (NTPs). In certain embodiments, the nucleosides or nucleotides are not dNTPs, or do not include dNTPs. In certain embodiments, administering nucleosides or nucleotides to the cell comprises administering the nucleosides or nucleotides to a subject comprising the cell. In certain embodiments, increasing the dNTP concentration in the cell comprises delivering a dNTP synthetic enzyme to the cell. In various aspects, the dNTP synthetic enzyme comprises a kinase. In certain embodiments, the kinase comprises a nucleoside kinase, deoxynucleoside kinase, deoxynucleoside monophsphase kinase, or deoxynucleotide diphosphate kinase. Such enhancers to increase availability of dNTP in a cell are known in the art.

In certain embodiments, a DNA repair protein-based enhancer is used in combination with an enhancer to increase the availability of dNTP in a cell.

In some embodiments, the presence of one or more enhancers may increase the efficiency of template-based genome editing by at least five-fold, optionally by at least 10-fold.

The term “chromatin remodeler,” as used herein, is a polypeptide that is capable of interacting with chromatin and mediating changes in chromatin. The chromatin remodeler may alter the interaction between DNA and DNA binding proteins, thereby increasing chromatin accessibility. In some embodiments, target genomic sequences located in a chromatin-dense region, in particular, may be less susceptible to genome editing. For example, it is known that Cas9 can exhibit reduced editing of targets whose protospacer adjacent motifs (PAMs) are located within nucleosomes. A system or fusion protein comprising a chromatin remodeler may therefore show increased editing efficiency at loci that are positioned within compacted chromatin.

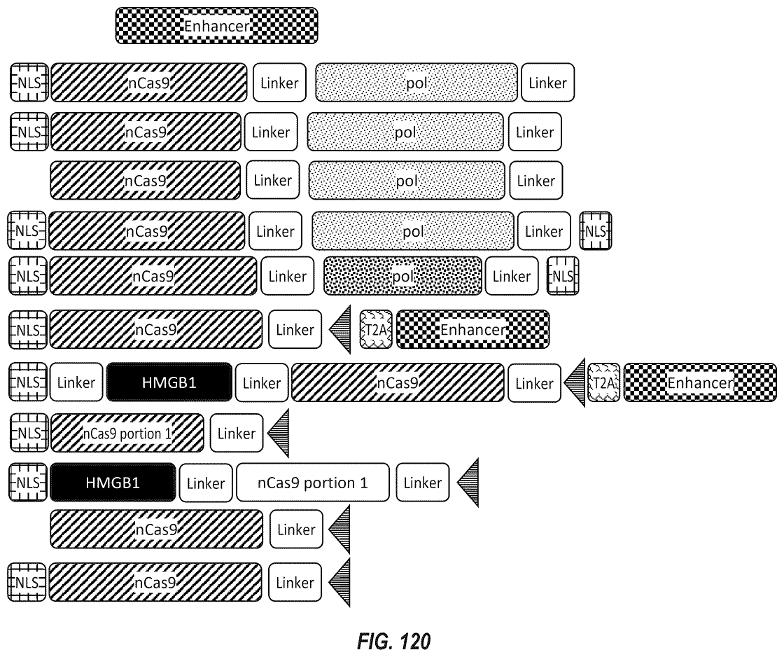

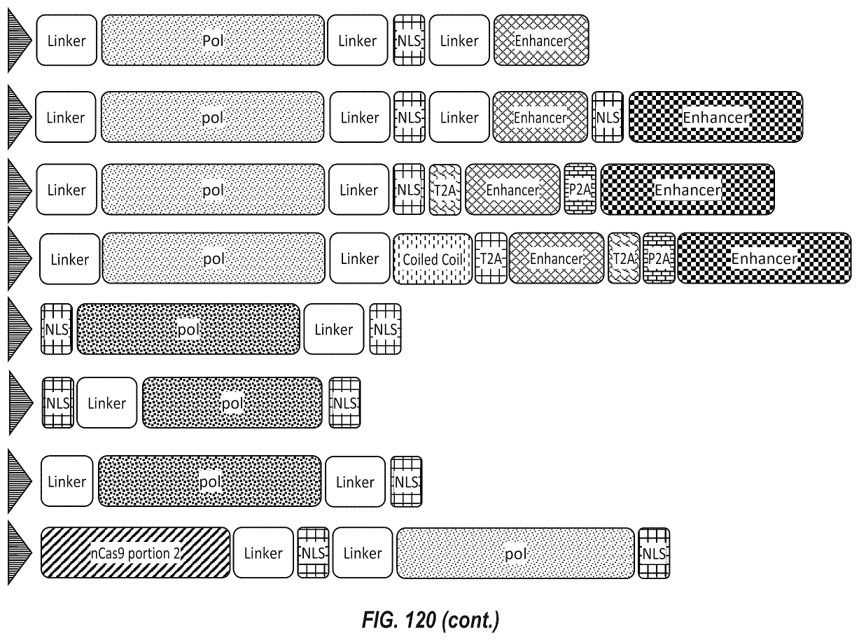

The term “High Mobility Group Box 1” or “HMGB1,” as used herein in the context of the HMGB1 protein, refers to a non-histone, nuclear DNA-binding protein belonging to the High Mobility Group-Box superfamily, or a portion thereof. The wildtype HMGB1 protein is composed of 215 amino acids in three structural domains: a Box A DNA-binding domain (alternatively referred to as an HMGB1 Box A domain elsewhere herein), an Box B DNA-binding domain (alternatively referred to elsewhere herein as an HMGB1 Box B domain elsewhere herein), and an acidic tail domain. The wildtype HMGB1 protein comprising SEQ ID NO: 1783 may be understood to comprise, from N-terminus to C-terminus, a HMGB1 Box A DNA-binding domain comprising the sequence of amino acid residues 2-78 of SEQ ID NO: 1783, a HMGB1 Box B DNA-binding domain comprising the sequence of amino acid residues 89-162 of SEQ ID NO: 1783, a cryptic nuclear localization signal (NLS) comprising the sequence of amino acid residues 179-185 of SEQ ID NO: 1783, and an acidic tail corresponding to amino acid residues 186-215 of SEQ ID NO: 1783. FIG. 120 shows an exemplary schematic of the wildtype HMGB1 protein. The term “HMGB1 polypeptide” as used herein refers to a polypeptide comprising the amino acid sequence of HMGB1, or a portion or fragment thereof. For example, in some embodiments, an HMGB1 polypeptide can comprise one or more HMGB1 domains (such as an HMGB1 Box B domain, an HMGB1 Box A domain, and an acidic tail domain). It is understood that, when an HMGB1 polypeptide is present as part of a fusion protein, the HMGB1 polypeptide may be referred as “HMGB1” (e.g., “a fusion protein comprising, from N-terminus to C-terminus, deaminase-first linker-DNA-binding domain-heterologous NLS-second linker-HMGB1”). In some embodiments, the acidic tail may serve as a transcription stimulatory domain. “HMGB1” as used herein in the context of nucleic acids refers to a nucleic acid (e.g., DNA or mRNA) encoding an HMGB1 polypeptide. The human HMGB1 gene has accession number NC_000013.11 (30456704 . . . 30617597). It is understood that, when an HMGB1 polypeptide (e.g., an HMGB1 protein of SEQ ID NO: 1783) is present as part of a fusion protein, the N-terminal methionine residue of HMGB1 may be omitted.

As used herein, in the context of the wildtype HMGB1 protein, the term “Box A” refers to an amino acid sequence comprising or consisting of SEQ ID NO: 1785. In some embodiments, the HMGB1 Box A domain may comprise or consist of amino acid residues 2-78 of SEQ ID NO: 1783. In some embodiments, the HMGB1 Box A domain may have DNA binding activity.

As used herein, in the context of the wildtype HMGB1 protein, the term “Box B” refers to an amino acid sequence comprising or consisting of SEQ ID NO: 1786. In some embodiments, the HMGB1 Box B domain may comprise or consist of amino acid residues 89-162 of SEQ ID NO: 1783. In some embodiments, the HMGB1 Box B domain may have DNA binding activity.

As used herein, in the context of the wildtype HMGB1 protein, the term “cryptic nuclear localization signal” or “cryptic NLS” refers to an amino acid sequence comprising or consisting of EKSKKKK (SEQ ID NO: 1787) or a variant thereof. In some embodiments, the cryptic NLS may comprise amino acid residues 179-185 of SEQ ID NO: 1783. In some embodiments, the cryptic NLS may comprise additional amino acid residues. In some embodiments, the cryptic NLS may comprise a part of or all of amino acid residues 166-185 of SEQ ID NO: 1783. In some embodiments, the cryptic NLS disclosed herein comprises a variant of EKSKKKK (SEQ ID NO: 1787) wherein one amino acid residue of SEQ ID NO: 1787 comprises a conservative substitution thereof. As used herein, a cryptic NLS, as provided by SEQ ID NO: 1787, or as a variant thereof, is not a heterologous NLS described herein.

In some embodiments, the cryptic NLS may have constitutive NLS activity (that is, act as a signal fragment that mediates the nuclear import of the HMGB1 protein). In some embodiments, the cryptic NLS may not have constitutive NLS activity.

As used herein, in the context of the wildtype HMGB1 protein, the term “receptor for advanced glycation end-products (RAGE) binding domain” or “RAGE binding domain” refers to an amino acid sequence comprising amino acid residues 150-183 of SEQ ID NO: 1783. See also FIG. 120. In some embodiments, the HMGB1 polypeptide described herein comprises a RAGE binding domain, or portion thereof, C-terminal to the HMGB1 Box B domain. In some embodiments, the RAGE binding domain comprises the amino acid sequence of SEQ ID NO: 1803 C terminal to the Box B domain.

As used herein, in the context of the wildtype HMGB1 protein, the term “acidic tail domain” of an amino acid sequence comprising or consisting of SEQ ID NO: 1788. In some embodiments, the acidic tail domain may comprise or consist of amino acid residues 186-215 of SEQ ID NO: 1783. In some embodiments, the acidic tail domain may have transcriptional stimulatory function.

As used herein, the term “fusion protein” (or “fusion polypeptide”) refers to a hybrid polypeptide which comprises polypeptides from at least two different proteins or sources. One polypeptide may be located at the amino-terminal (N-terminal) portion of the fusion protein or at the carboxy-terminal (C-terminal) protein thus forming an “amino-terminal fusion protein” or a “carboxy-terminal fusion protein,” respectively. It is understood that, if one or more of the polypeptides includes an N-terminal methionine corresponding to a start codon, the methionine may be omitted from the fusion protein. For example, a polypeptide that is not located at the N-terminal portion in some cases may lack an N-terminal methionine and its coding sequence may similarly lack a start codon in its open reading frame that ordinarily would encode an N-terminal methionine. Any of the proteins provided herein may be produced by any method known in the art. For example, a fusion protein may be expressed in the cell where the activity of the fusion protein is desired, e.g., in a mammalian cell from an mRNA or expression vector. A fusion protein may be present in a cell. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual 4th ed., Cold Spring Harbor Laboratory Press, Cold Spring Harbor, N.Y. (2012)), the entire contents of which are incorporated herein by reference.

The term “linker,” as used herein, refers to a chemical group or a molecule linking two adjacent molecules or moieties. Typically, the linker is positioned between, or flanked by, two groups, molecules, or other moieties and connected to each one via a covalent bond. In some embodiments, the linker is a peptide linker comprising an amino acid or a plurality of amino acids (e.g., a peptide or protein) such as a 16-amino acid residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 1601), SGSETPGTSESA (SEQ ID NO: 1602), SGSETPGTSESATPEGGSGGS (SEQ ID NO: 1603), or GAPESATESGGTSTESEGSAGPKKKRKVTSTESEGSAGSAGSTSGSSSG (SEQ ID NO: 1600). In some embodiments, the linker comprises an amino acid sequence selected from SEQ ID NOs: 1596-1603, 1604-1665, 1735, 1738, 1815, 1825, 1827, and 1829-1830.

The term “intein,” as used herein refers to a protein domain capable of mediating a process known as protein splicing. In certain instances, the intein may be one formed from two separate but complementary split inteins that are capable of ligating together to mediate a trans-protein splicing reaction. The term “split intein” refers to an intein that may exist as two separate polypeptide fragments—an N-split-intein (N-intein) and a C-split-intein (C-intein)—where the two polypeptide fragments are capable of ligating together and undergoing a protein trans-splicing reaction culminating in the formation of a peptide bond between polypeptide sequences flanking each split intein. For example, a first split intein positioned at the C-terminus of a first polypeptide (the N-terminal intein or N-intein) and a second split intein positioned at the N-terminus of a second polypeptide (the C-terminal intein or C-intein) may undergo a chemical reaction culminating in the formation of a peptide bond between the first polypeptide and the second polypeptide and the excision of the first and second intein domains. Intein-mediated protein splicing is known in the art, and represents a well-established technique for generating a single protein from two separate polypeptides (see, e.g., Shah and Muir. Inteins: Nature's Gift to Protein Chemists. Chem. Sci., 5 (1), 446-461 (2014)).

The term “heterodimerization domain” can include coiled coil polypeptides capable of self-assembling into stable heterodimers with other coiled coil polypeptides referred to herein as “coiled coil heterodimerization domains.” The coiled coil heterodimerization domain is a ubiquitous protein folding and assembly motif which has been identified in well over 200 natural proteins. Structurally, it is made of α-helices wrapping around each other forming a supercoil, either right- or left-handed. Such structures are well known in the art. In some embodiments, coiled coil heterodimerization domains may comprise EV, EI, KV, and KI domains, where E, V, I, and K are aspartic acid, valine, isoleucine, and lysine, respectively. Exemplary coiled coil heterodimerization domains may comprise, from N-terminus to C-terminus, the sequence EVSALEKEVSALEKENSALEWEVSALEK (SEQ ID NO: 1590), KVSALKEKVSALKEKNSALKWKVSALKE (KV domain, SEQ ID NO: 1591), IAALEKEIAALEKENAALEWEIAALEK (EI domain, SEQ ID NO: 1589), EIAALEKEIAALEKENAALEWEIAALEK (EI domain, SEQ ID NO: 1593), or KIAALKEKIAALKEKNAALKWKIAALKE (KI domain, SEQ ID NO: 1595). Coiled coil heterodimerization domains are known in the art and associate with their binding domain (see, e.g., Aronsson C, Dånmark S, Zhou F, Öberg P, Enander K, Su H, Aili D. Self-sorting heterodimeric coiled coil heterodimerization domains with defined and tuneable self-assembly properties. Sci Rep. 2015 Sep. 15; 5:14063. doi: 10.1038/srep14063). KI domains interact with EI domains to form heterodimers, while KV domains interact with EV domains to form heterodimers.

As used herein, the terms “nuclear localization signal” (NLS) or “nuclear localization sequence” refers to an amino acid sequence which induces transport of molecules comprising such sequences or linked to such sequences into the nucleus of eukaryotic cells. The nuclear localization signal may form part of the molecule to be transported. In some embodiments, the NLS may be fused to the molecule by a covalent bond, hydrogen bonds, or ionic interactions. In some embodiments, the NLS may be fused to the molecule via a linker.

As used herein, the term “genomic locus,” when used in the context of a genomic locus being targeted by a guide RNA, includes one or more parts of a genome, the targeting of which affects the expression of the gene that is associated with the locus. For example, a genomic locus may include a coding sequence of a gene, an intron sequence of a gene, a regulatory sequence, a transcriptional control sequence of a gene, a translational control sequence of a gene, a splicing site, or a non-coding sequence between genes (e.g., intergenic space).

As used herein, a “target sequence” or “genomic target sequence” refers to a sequence of nucleic acid in a target genomic locus, in either the positive or the negative strand, that has complementarity to the guide sequence of the guide RNA, i.e., that is sufficiently complementary to the guide sequence of the guide RNA to permit specific binding of the guide to the target sequence. The interaction of the target sequence and the guide scaffold sequence directs an RNA-guided DNA binding agent, e.g., a SpyCas9 nickase, to bind, and potentially nick or cleave (depending on the activity of the agent), within the target sequence. The specific length of the target sequence and the number of mismatches possible between the target sequence and the guide sequence depend, for example, on the identity of the Cas9 nickase being directed by the guide RNA. Target sequences for Cas proteins include both the positive and negative strands of genomic DNA (i.e., the sequence given and the sequence's complement), as a nucleic acid substrate for a Cas protein is a double stranded nucleic acid. Accordingly, where a guide sequence is said to be “complementary to a target sequence,” it is to be understood that the guide sequence may direct SpyCas9 nickase to bind to the complement of a target sequence. Thus, in some embodiments, where the guide sequence binds the complement of a target sequence, the guide sequence is identical to certain nucleotides of the target sequence (e.g., the target sequence which does not include the PAM) except for the substitution of U for T in the guide sequence.

In some embodiments, the target sequence is in a genomic locus or on a chromosome, for example, and is complementary to the guide sequence. In some embodiments, the degree of complementarity or identity between a guide sequence and its corresponding target sequence may be about 80%, 85%, preferably about 90%, 95%, or 100%. In some embodiments, the guide sequence and the target sequence may be 100% complementary or identical. In other embodiments, the guide sequence and the target sequence may contain at least one mismatch. For example, the guide sequence and the target sequence may contain 1, 2, 3, or 4 mismatches, where the total length of the duplex formed between the guide sequence and the target sequence in the genome is typically across at least 20 base pairs, with mismatches possible across the base paired region. In some embodiments, the degree of complementarity or identity between a guide sequence and its corresponding target sequence is at least 80%, 85%, preferably at least 90%, or 95%, for example when, the guide sequence comprises a sequence 20 consecutive nucleotides. In other embodiments, the guide sequence and the target sequence may contain at least one mismatch, i.e., one nucleotide that is not identical or not complementary, depending on the reference sequence.

As used herein, a “target strand” refers to the strand (positive or negative) of the DNA duplex target nucleic acid that comprises the target sequence.

As used herein, a “non-target strand” refers to the strand (positive or negative) of the DNA duplex target nucleic acid that comprises a sequence that is complementary to the target sequence.

As used herein, a “first sequence” is considered to “comprise a sequence that is at least X % identical to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

As used herein, “knockdown” or “knockout” refers to a decrease or loss in expression of a particular gene product (e.g., protein, mRNA, or both). Knockdown of a protein can be measured either by detecting protein secreted by tissue or population of cells (e.g., in serum or cell media) or by detecting total cellular amount of the protein from a tissue or cell population of interest. Methods for measuring knockdown of mRNA are known and include sequencing of mRNA isolated from a tissue or cell population of interest. In some embodiments, “knockdown” may refer to some loss of expression of a particular gene product, for example a decrease in the amount of mRNA transcribed or a decrease in the amount of protein expressed or secreted by a population of cells (including in vivo populations such as those found in tissues). In some embodiments, “knockdown” may refer to a decrease of expression to below the level of detection of the assay used.

As used herein, “reduces or eliminates” (or “reduced or eliminated”) expression of a protein on a cell refers to a partial or complete loss (i.e., to below the limit of detection of the assay used) of expression of the protein, respectively, relative to an unmodified cell. The terms can be used individually or in conjunction. In some embodiments, the surface expression of a protein on a cell is measured by flow cytometry and has “reduced or eliminated” surface expression relative to an unmodified cell as evidenced by a reduction in fluorescence signal upon staining with the same antibody against the protein. A cell that has “eliminated” surface expression of a protein by flow cytometry relative to an unmodified cell may be referred to as “negative” for expression of that protein as evidenced by a fluorescence signal similar to a cell stained with an isotype control antibody. The “reduction or elimination” of protein expression can be measured by other known techniques in the field with appropriate controls known to those skilled in the art, e.g., western blot, ELISA, or immunohistochemistry. In certain embodiments, expression is assayed in a subject sample, e.g., a tissue or body fluid, e.g., urine, blood, or serum or plasma derived therefrom. In certain embodiments, reduction of expression can be assessed in the same subject sample that may not represent reduction at all sites of expression systemically. For example, a protein may be expressed in multiple tissues, but treatment results in reduction of expression predominantly in a single tissue, e.g., in liver. In certain embodiments, reduction can be assessed by an activity assay, or by the level of a surrogate marker present in a subject sample, e.g., urine or blood, that correlates with reduction of protein expression. In certain embodiments, reduction can include a greater reduction of expression of a protein containing a mutation as compared to reduction of expression of the wild-type protein in a subject heterozygous at a particular genomic sequence. In certain embodiments, reduction can include reduction of expression of a protein containing a mutation relative to expression of the wild-type protein. The ratio may be changed, for example, by preferentially reducing the expression of the protein containing a mutation or altering the genomic sequence encoding the protein containing a mutation to the wild-type sequence. In certain embodiments, reduction can include reducing expression of an endogenous protein relative to expression of a protein having a desired polypeptide sequence that may not be a wild-type protein. As used herein, “eliminated” expression is understood as a reduction of expression to below the level of detection of the protein by the method used. It is understood that reduction of expression can include elimination of expression.

As used herein, “increased” (or “increases”) expression of a protein on a cell refers to an increase in expression of the protein relative to an unmodified cell. In some embodiments, the surface expression of a protein on a cell is measured by flow cytometry and has “increased” surface expression relative to an unmodified cell as evidenced by an increase in fluorescence signal upon staining with the same antibody against the protein. The “increase” of protein expression can be measured by other known techniques in the field with appropriate controls known to those skilled in the art, e.g., western blot, ELISA, or immunohistochemistry. In certain embodiments, expression is assayed in a subject sample, e.g., a tissue or body fluid, e.g., urine, blood, or serum or plasma derived therefrom. In certain embodiments, increase of expression can be assessed in the same subject sample that may not represent increase in all sites of expression systemically. For example, a protein may be expressed in multiple tissues, but treatment results in reduction of expression predominantly in a single tissue, e.g., in liver. In certain embodiments, increase can be assessed by an activity assay, or by the level of a surrogate marker present in a subject sample, e.g., urine or blood, that correlates with an increase of protein expression. In certain embodiments, increase can include a greater increase of expression of a protein a wild-type protein as compared to expression of the protein containing a mutation as in a subject heterozygous at a particular genomic sequence. In certain embodiments, increase can include increase of expression of a wild-type protein relative to expression of the protein containing a mutation. The ratio may be changed, for example, by preferentially increasing the expression of the wild-type protein by altering the genomic sequence encoding the protein containing a mutation to the wild-type sequence.

“Messenger RNA” or “mRNA” is used herein to refer to a polynucleotide that comprises an open reading frame that can be translated into a polypeptide (i.e., can serve as a substrate for translation by a ribosome and amino-acylated tRNAs). mRNA can comprise one or more chemically modified nucleosides such as 5-methyl-cytidine (5mC), 2-thio-uridine (2sU), N1-methylpseudouridine (ml VU) and pseudo-uridine (U), or a modified cap structure as provided below.

As used herein, “open reading frame” or “ORF” of a gene refers to a sequence consisting of a series of codons that specify the amino acid sequence of the protein that the gene codes for. The ORF generally begins with a start codon (e.g., ATG in DNA or AUG in RNA) and ends with a stop codon, e.g., TAA, TAG or TGA in DNA or UAA, UAG, or UGA in RNA. The ORF sequences described herein may or may not include a start codon encoding an N-terminal methionine. Thus, in some cases, an ORF described in a SEQ ID NO herein comprises a start codon and the ORF includes the start codon. In other cases, an ORF comprises the sequence of the listed SEQ ID NO but without the start codon provided in the sequence of the listed SEQ ID NO. In some cases, an ORF described in a SEQ ID NO herein comprises a stop codon and the ORF includes the stop codon. In other cases, an ORF comprises the sequence of the listed SEQ ID NO but without the stop codon provided in the sequence of the listed SEQ ID NO.

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy, 2′ halide, or 2′-O-(2-methoxyethyl) (2′-O-moe) substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N4-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, 06-methylguanine, 4-thio-pyrimidines, 4-amino-pyrimidines, 4-dimethylhydrazine-pyrimidines, and 04-alkyl-pyrimidines; U.S. Pat. No. 5,378,825 and PCT No. WO 93/13121). For general discussion see The Biochemistry of the Nucleic Acids 5-36, Adams et al., ed., 11th ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). Nucleic acid includes “unlocked nucleic acid” enables the modulation of the thermodynamic stability and also provides nuclease stability. RNA and DNA have different sugar moieties and can differ by the presence of uridine or analogs thereof in RNA and thymine or analogs thereof in DNA. It is understood that depending on the function of the polynucleotide, certain nucleosides or nucleoside analogs may be preferred, or certain nucleosides or nucleoside analogs may not be tolerated.

As used herein, a “population of cells comprising edited cells” (or “population of cells comprising engineered cells”) or the like refers to a cell population that comprises edited cells (or engineered cells), however not all cells in the population must be edited. A cell population comprising edited cells may also include non-edited cells. The percentage of edited cells within a cell population comprising edited cells may be determined by counting the number of cells within the population that are edited in the population as determined by standard cell counting methods, e.g., cell sorting, immunohistochemistry, NGS. For example, in some embodiments, a cell population comprising edited cells comprising a single genome edit will have at least 5%, 10%, 15%, 20%, 25%, 30%, 35%, 40%, 45%, preferably at least 50%, 60%, 70%, 80%, 90%, or 95% of the cells in the population with the single edit. As used herein, the population of cells comprising edited cells can be a population of cells comprising a template-based edited cells. In certain embodiments, the template-based edited cells may further include non-template based edits, e.g., byproduct edits not encoded by the template.

As used herein, a “subject” refers to any member of the animal kingdom. In some embodiments, “subject” refers to humans. In some embodiments, “subject” refers to non-human animals. In some embodiments, “subject” refers to primates. In some embodiments, a subject may be a transgenic animal, genetically engineered animal, or a clone. In certain embodiments of the present disclosure the subject is an adult, an adolescent, or an infant. In some embodiments, terms “individual” or “patient” are used and are intended to be interchangeable with “subject”.

As used herein, a “control” is understood as an appropriate matched sample or subject for comparison. For example, a control can be a cell population treated in the same manner as the test population except that the treatment used for the control population lacks at least one active agent, e.g., a guide RNA, an mRNA encoding a SpyCas9 nickase, an insertion construct, a lipid formulation. In certain embodiments, a control may be an internal control, e.g., a cell population or subject prior to treatment.

As used herein, the term “contact” refers to providing at least one component so that the component physically contacts a cell, including physically contacting the cell surface, cytosol, or nucleus of the cell. “Contacting” a cell with a polypeptide encompasses, for example, contacting the cell with a nucleic acid that encodes the polypeptide and allowing the cell to express the polypeptide.

As used herein, “delivering” and “administering” are used interchangeably, and include ex vivo and in vivo applications.

Co-administration, as used herein, means that a plurality of substances are administered sufficiently close together in time so that the agents act together. Co-administration encompasses administering substances together in a single formulation and administering substances in separate formulations close enough in time so that the agents act together.

As used herein, “treatment” refers to any administration or application of a therapeutic for disease or disorder in a subject, and includes inhibiting the disease, arresting its development, relieving one or more symptoms of the disease, curing the disease, or preventing one or more symptoms of the disease, including reoccurrence of the symptom.

As used herein, the phrase “pharmaceutically acceptable” means that which is useful in preparing a pharmaceutical composition that is generally non-toxic and is not biologically undesirable and that are not otherwise unacceptable for pharmaceutical use. Pharmaceutically acceptable generally refers to substances that are non-pyrogenic. Pharmaceutically acceptable can refer to substances that are sterile, especially for pharmaceutical substances that are for injection or infusion.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

The term “about” or “approximately” means an acceptable error for a particular value as determined by one of ordinary skill in the art, which depends in part on how the value is measured or determined, or a degree of variation that does not substantially affect the properties of the described subject matter, or within the tolerances accepted in the art, e.g., within 10%, 5%, 2%, or 1% or within two standard deviations of a set of values. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques. When “about” is present before the first value of a series, it is understood to modify each value in the series.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The term “or” is used in an inclusive sense, i.e., equivalent to “and/or” unless the context clearly indicates otherwise.

Ranges are understood to include the numbers at the end of the range and all logical values therebetween. For example, 5-10 nucleotides is understood as 5, 6, 7, 8, 9, or 10 nucleotides, whereas 5-10% is understood to contain 5% and all possible values through 10%.

At least 17 nucleotides of a 20 nucleotide sequence is understood to include 17, 18, 19, or 20 nucleotides of the sequence provided, thereby providing a upper limit even if one is not specifically provided as it would be clearly understood. Similarly, up to 3 nucleotides would be understood to encompass 0, 1, 2, or 3 nucleotides, providing a lower limit even if one is not specifically provided. When “at least,” “up to,” or other similar language modifies a number, it is understood to modify each number in the series.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

In the event of a conflict between a sequence in the application and an indicated accession number or position in an accession number, the sequence in the application predominates.

As used herein, “detecting an analyte” and the like is understood as performing an assay in which the analyte can be detected, if present, wherein the analyte is present in an amount above the level of detection of the assay.

As used herein, it is understood that when the maximum amount of a value is represented by 100% (e.g., 100% inhibition or 100% encapsulation) that the value is limited by the method of detection. For example, 100% inhibition is understood as inhibition to a level below the level of detection of the assay, and 100% encapsulation is understood as no material intended for encapsulation can be detected outside the vesicles.

As used herein, “consecutive” is understood as following one after another without interruption; successive, contiguous.

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

### Template Guide RNA

Provided here are a template guide RNA (tgRNA) comprising a first polynucleotide and a second polynucleotide, wherein: (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; and (ii) a DNA-dependent DNA polymerase recruiting sequence (DRS) having a 3′ end and a 5′ end; wherein the 5′ end of the DRS is covalently attached to the 3′ end of the template sequence, and wherein the DRS comprises a sequence that is reverse complementary to at least 6 nucleotides of a DRS-complementary region of the non-target strand of the DNA duplex target nucleic acid. In some embodiments, the DRS is 6-20 nucleotides in length, optionally 10-14 nucleotides in length. In some embodiments, the DRS comprises at least one of: (a) a sequence not fully complementary to the DRS-complementary region of the non-target strand of the DNA duplex target nucleic acid; (b) a 1-5 nucleotide, optionally a 1-3 nucleotide 3′ terminal tail comprising a 3′ terminal modified uridine nucleotide; or a terminal (D1) DNA nucleotide and a penultimate 5′ terminal (D2) RNA nucleotide wherein the 3′ end of the first polynucleotide is covalently linked to the 5′ end of the second polynucleotide by a phosphorothioate or phosphodiester linkage. In some embodiments, the DRS of the tgRNA comprises DNA nucleotides. In some embodiments, the DRS of the tgRNA comprises both RNA nucleotide(s) and DNA nucleotide(s).

Provided here are systems comprising a template guide RNA (tgRNA) comprising a first polynucleotide and a second polynucleotide, wherein: (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; and (ii) a DNA-dependent DNA polymerase recruiting sequence (DRS) having a 3′ end and a 5′ end; wherein the 5′ end of the DRS is covalently attached to the 3′ end of the template sequence, and wherein the DRS comprises a sequence that is reverse complementary to at least 6 nucleotides of a DRS-complementary region of the non-target strand of the DNA duplex target nucleic acid, wherein the DRS comprises RNA nucleotides.

In some embodiments, the tgRNA comprises a first polynucleotide and a second polynucleotide, wherein: (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; (ii) a DNA-dependent DNA polymerase recruiting sequence (DRS) that is 6-20 nucleotides in length, optionally 6-17 or 10-14 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid; and wherein the DRS comprises at least one of: (1) a sequence not fully complementary to the DRS-complementary region of the non-target strand of the DNA duplex target nucleic acid; (2) a 1-5, optionally a 1-3 nucleotide 3′ terminal tail comprising a 3′ terminal modified uridine nucleotide; or (3) a 5′ terminal (D1) DNA nucleotide and a penultimate 5′ terminal (D2) RNA nucleotide wherein the 3′ end of the first polynucleotide is covalently linked to the 5′ end of the second polynucleotide by a phosphorothioate or phosphodiester linkage. In some embodiments, the DRS of the tgRNA comprises DNA nucleotides. In some embodiments, the DRS of the tgRNA comprises both RNA nucleotide(s) and DNA nucleotide(s).

In some embodiments, the tgRNA comprises a first polynucleotide and a second polynucleotide, wherein: (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; (ii) a DNA-dependent DNA polymerase recruiting sequence (DRS) that is 6-20 nucleotides in length, optionally 6-17 or 10-14 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid, wherein the DRS comprises RNA nucleotides; and wherein the DRS comprises at least one of: (1) a sequence not fully complementary to the DRS-complementary region of the non-target strand of the DNA duplex target nucleic acid; (2) a 1-5, optionally a 1-3 nucleotide 3′ terminal tail comprising a 3′ terminal modified uridine nucleotide; or (3) a 5′ terminal (D1) DNA nucleotide and a penultimate 5′ terminal (D2) RNA nucleotide wherein the 3′ end of the first polynucleotide is covalently linked to the 5′ end of the second polynucleotide by a phosphorothioate or phosphodiester linkage.

In some embodiments, the tgRNA comprises a first polynucleotide and a second polynucleotide, wherein (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; (ii) a DNA-dependent DNA polymerase recruiting sequence (DRS) that is about 6-18 nucleotides in length, optionally about 8-18 or 10-12 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid. In some embodiments, the DRS of the tgRNA comprises DNA nucleotides. In some embodiments, the DRS of the tgRNA comprises both RNA nucleotide(s) and DNA nucleotide(s).

In some embodiments, the tgRNA comprises a first polynucleotide and a second polynucleotide, wherein (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; (ii) a DNA-dependent DNA polymerase recruiting sequence (DRS) that is about 6-18 nucleotides in length, optionally about 8-18 or 10-12 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid, wherein the DRS comprises RNA nucleotides.

In some embodiments, the tgRNA comprises a first polynucleotide and a second polynucleotide, wherein (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; and (ii) a DNA-dependent DNA polymerase recruiting sequence (DRS) that is 6-20 nucleotides in length, optionally 6-17 or 10-14 nucleotides in length, and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid comprising nucleotides 5-10 5′ of the PAM in the non-target strand of the DNA duplex target nucleic acid. In some embodiments, the DRS of the tgRNA comprises DNA nucleotides. In some embodiments, the DRS of the tgRNA comprises both RNA nucleotide(s) and DNA nucleotide(s).

In some embodiments, the tgRNA comprises a first polynucleotide and a second polynucleotide, wherein (a) the first polynucleotide comprises a SpyCas9 guide RNA comprising: (i) a spacer that is complementary to a target region of a target strand of a DNA duplex target nucleic acid; and (ii) a scaffold; and (b) the second polynucleotide is operably linked to the first polynucleotide and comprises: (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; and (ii) a DNA-dependent DNA polymerase recruiting sequence (DRS) that is 6-20 nucleotides in length, optionally 6-17 or 10-14 nucleotides in length, and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid comprising nucleotides 5-10 5′ of the PAM in the non-target strand of the DNA duplex target nucleic acid, wherein the DRS comprises RNA nucleotides.

In some embodiments, the first region in the target strand and the second region in the non-target strand each base pair with a strand of the DNA duplex independently for 6-20 consecutive nucleotides within the spacer genomic target sequence. In some embodiments, the first region in the target strand and the second region in the non-target strand base pair with a strand of the DNA duplex independently for 6-16 consecutive nucleotides within the spacer genomic target sequence. In some embodiments, the first region in the target strand and the second region in the non-target strand base pair with a strand of the DNA duplex independently for 6-10 consecutive nucleotides within the spacer genomic target sequence. In some embodiments, the first region in the target strand and the second region in the non-target strand each base pair with a strand the DNA duplex independently for at least 6, 7, 8, 9, 10, 11, 12, 13, 14, 15, 16, 17, 18, 19, or 20 consecutive nucleotides within the spacer genomic target sequence.

In some embodiments, the first polynucleotide is covalently linked to the second polynucleotide. In some embodiments, the 3′ end of the first polynucleotide is covalently linked to the 5′ end of the second polynucleotide. In some embodiments, the 3′ end of the first polynucleotide is covalently linked to the 5′ end of the second polynucleotide by a phosphodiester bond or a phosphorothioate (PS) bond. In some embodiments, the 3′ end of the first polynucleotide is covalently linked to the 5′ end of the second polynucleotide by a phosphodiester bond. In some embodiments, the 3′ end of the first polynucleotide is covalently linked to the 5′ end of the second polynucleotide by a phosphorothioate bond. In some embodiments, the first polynucleotide is linked to the second polynucleotide by a non-nucleotide linker. In some embodiments, the first polynucleotide is linked to the second polynucleotide by an internal linker. In some embodiments, the 3′ end of the first polynucleotide is covalently linked to the 5′ end of the second polynucleotide by an internal linker.

Provided herein are guide RNAs that in some embodiments include an operably linked 3′ extension sequence, referred to herein as template guides (tgRNA). As used herein, a guide RNA (gRNA) is understood to include at least a spacer (targeting) sequence that is complementary to a target sequence in a genomic locus; and a scaffold sequence for binding a SpyCas9 nickase. The spacer and scaffold can be in a single polynucleotide, i.e., a single guide RNA (sgRNA), or in two polynucleotides, i.e., a dual guide RNA (dgRNA) in which the crRNA and tracrRNA are separate polynucleotides.

In certain embodiments, the guide RNA may be operably linked to a 3′-extension (i.e., a template guide RNA (tgRNA)). A 3′ extension includes, 5′ to 3′, a template and a DNA polymerase recruiting sequence (DRS). The template and DRS are covalently linked by a polynucleotide backbone linkage, e.g., a phosphodiester or phosphorothioate linkage, preferably a phosphorothioate linkage. When covalently attached, the 3′ extension is covalently attached, directly or indirectly, to the 3′ end of the scaffold. When the 3′ extension is not covalently attached to the 3′ end of the scaffold, it is referred to herein as a separable 3′ extension as it provides the same features at a site of template-based genome editing. When the 3′ extension is not covalently linked to the 3′ end of the scaffold, it can be retained at the site of template-based genome editing by binding to the polymerase, e.g., via an affinity tag to a 3′ extension-recruiting domain in the polymerase.

It is understood that each the spacer and scaffold, the template, and the DRS have distinct functions. These distinct functions result in different tolerances for various nucleotides including chemically modified nucleotides and are provided herein. Therefore, as discussed herein, the terms “unmodified nucleotide” or “modified nucleotide” can be understood differently depending on the particular structure in which the nucleotide is present. Similarly, modified nucleotides for inclusion in an ORF or mRNA are different from those for inclusion in a guide RNA. Such considerations are well understood in the art.

In some embodiments, the first polynucleotide sequence and the second polynucleotide sequence are provided as part of separate polynucleotide chains (i.e., the 3′ extension is a separable 3′ extension). In some embodiments, the separate polynucleotide chains comprising the first and second polynucleotide sequences, optionally with the first polynucleotide sequence complexed with SpyCas9 and the second polynucleotide sequence complexed with a DNA-dependent DNA polymerase, may be operably linked by being targeted to at least partially complementary sequences on opposite DNA strands of a genomic locus, i.e., nucleotide sequences that would base pair in the DNA duplex in the absence of disruption of the duplex, e.g., by a tgRNA. In some embodiments, the second polynucleotide sequence may be complexed to the DNA-dependent DNA polymerase through an affinity tag that binds to a corresponding domain on the DNA-dependent DNA polymerase.

In some embodiments, the separate polynucleotide chains comprising the first and second polynucleotide sequences may be operably linked by forming a complex with a single polypeptide or a single polypeptide heterodimer, optionally wherein the polypeptide or single polypeptide heterodimer comprises SpyCas9 and a DNA-dependent DNA polymerase. In some embodiments, the first polynucleotide sequence may be complexed with SpyCas9 and the second polynucleotide sequence may be complexed with the DNA-dependent DNA polymerase. In some embodiments, the second sequence may be complexed with the DNA-dependent DNA polymerase through an affinity tag that binds to a corresponding 3′ extension recruiting domain on the single polypeptide or single polypeptide heterodimer. Guide RNAs

### Target/Spacer Sequences and Genes

In some embodiments, the methods and compositions of the present disclosure utilize a SpyCas9 nickase, and a SpyCas9 guide RNA. In some embodiments, the target region of the target strand complementary to the spacer of the SpyCas9 guide RNA is complementary to the non-target region of the non-target strand. The non-target region of the non-target strand, in turn, may be complementary to the DRS independently over at least six consecutive nucleotides, e.g. over 6-20 consecutive nucleotides, e.g., over 5-20, 2-10, or 2-16 consecutive nucleotides relative to the target site of the spacer genomic target sequence in the DNA duplex target nucleic acid (See, e.g., FIGS. 2A and B). In other words, the sequence of the target region of the target strand may be identical to or partially identical to the sequence of the DRS independently over at least six consecutive nucleotides, e.g., over 6-20 consecutive nucleotides, e.g., over 5-20 2-10, or 2-16 consecutive nucleotides. In some embodiments, the SpyCas9 guide RNA spacer and scaffold target the SpyCas9 nickase to nick the non-target strand of the DNA duplex target nucleic acid.

For example, a non-target strand of a DNA duplex target nucleic acid may be nicked by a SpyCas9 nickase by being directed to the target site by a guide RNA. A target sequence for a Cas nuclease is located near its cognate PAM sequence. A SpyCas9 nickase may be directed by a guide RNA to a target sequence of a gene, where the guide RNA hybridizes with and the SpyCas9 nicks the non-target strand adjacent to its cognate PAM. The target sequence may be complementary or have identity to a spacer sequence (i.e., targeting sequence) of the guide RNA, and the cleavage may occur on one or both strands, preferably on a non-target strand, depending on the activity of the SpyCas9 nuclease. In some embodiments, the target sequence may have complementarity to a spacer sequence (i.e., targeting sequence) of the guide RNA, and the cleavage may occur on a non-target strand. In some embodiments, the degree of complementarity between a targeting sequence of a guide RNA and the portion of the corresponding target sequence that hybridizes to the guide RNA may be about 80%, 85%, preferably about 90%, 95%, or 100%. In some embodiments, the percent identity between a targeting sequence of a guide RNA and the portion of the corresponding target sequence that hybridizes to the guide RNA may be about 80%, 85%, preferably about 90%, 95%, or 100%. The homology region of the target is adjacent to a cognate PAM sequence. In some embodiments, the target sequence may comprise a sequence 100% complementary or 100% identical with the targeting sequence of the guide RNA. In other embodiments, the target sequence may comprise at least one mismatch, deletion, or insertion, as compared to the targeting sequence of the guide RNA.

The length of the target sequence may depend on the nuclease system used. For example, the targeting sequence of a guide RNA for a CRISPR/SpyCas9 system may be 16, 17, 18, 19, 20, 21, or 22 nucleotides in length and the target sequence is a corresponding length, adjacent to a PAM sequence. In some embodiments, the target sequence is 16-22 nucleotides in length. In preferred embodiments, the target sequence is 18-20 nucleotides in length, e.g., 20 nucleotides in length.

The target nucleic acid molecule may be any DNA molecule that is endogenous or exogenous to a cell, e.g., a DNA molecule of a pathogen, e.g., a virus. In some embodiments, the target nucleic acid molecule is a genomic DNA. In some embodiments, the target sequence of the gene may be a genomic sequence from a cell or in a cell, including a human cell. In some embodiments, the target sequence of the gene may be a genomic sequence from a pathogen.

In further embodiments, the target sequence may be a viral sequence. In further embodiments, the target sequence may be a pathogen sequence. In further embodiments, the target sequence may be a genomic sequence. In certain embodiments, the target sequence may comprise a translocation junction, e.g., a translocation associated with a cancer. In some embodiments, the target sequence may be on a eukaryotic chromosome, such as a human chromosome.

In some embodiments, the target sequence may be located in a genomic locus; for example, the target sequence may be located in a coding sequence of a gene, an intron sequence of a gene, a regulatory sequence, a transcriptional control sequence of a gene, a translational control sequence of a gene, a splicing site, or a non-coding sequence between genes (e.g., intergenic space). In some embodiments, the gene may be a protein coding gene. In other embodiments, the genomic locus may be at a non-coding locus. In some embodiments, the target sequence may be in a disease-associated gene. In some embodiments, the target sequence may be located in a non-genic functional site in the genome, for example a site that controls aspects of chromatin organization, such as a scaffold site or locus control region.

In some embodiments involving a Cas nuclease, such as a Class 2 Cas nuclease, the target sequence may be adjacent to a protospacer adjacent motif (“PAM”). In some embodiments, the PAM may be adjacent to or within 1, 2, 3, or 4, nucleotides of the 3′ end of the target sequence. The length and the sequence of the PAM may depend on the Cas protein used. For example, the PAM may be selected from a consensus or a particular PAM sequence for a specific SpyCas9 protein or SpyCas9 ortholog, including those disclosed in FIG. 1 of Ran et al., Nature, 520:186-191 (2015), and Figure S5 of Zetsche 2015, the relevant disclosure of each of which is incorporated herein by reference. In some embodiments, the PAM may be 2, 3, 4, 5, 6, 7, 8, 9, or 10 nucleotides in length. Non-limiting exemplary PAM sequences include NGG, NGGNG, NG, NAAAAN, NNAAAAW, NNNNACA, GNNNCNNA, TTN, and NNNNGATT (wherein N is defined as any nucleotide, and W is defined as either A or T). In some embodiments, the PAM sequence may be NGG. In some embodiments, the PAM sequence may be NGGNG. In some embodiments, the PAM sequence may be TTN. In some embodiments, the PAM sequence may be NNAAAAW. It is understood that SpyCas9 nucleases can be modified to alter PAM recognition. It is understood that the use of SpyCas9 nuclease with altered PAM recognition is within the scope of the disclosure provided herein.

### Guide RNA Scaffolds

In some embodiments, the guide RNA is a SpyCas9 guide RNA. In the case of a SpyCas9 single guide RNA (sgRNA), the above guide sequences may further comprise additional nucleotides to form a sgRNA, e.g., with the following exemplary nucleotide sequence following the 3′ end of the guide sequence:

(SEQ ID NO: 22)
GUUUUAGAGCUAGAAAUAGCAAGUUAAAAUAAGGCUAGUCCGUUAUCAA
CUUGAAAAAGUGGCACCGAGUCGGUGC in 5′ to 3′
orientation.

In the case of a sgRNA, the above guide sequences may further comprise additional nucleotides to form a sgRNA, e.g., with any one of the following exemplary nucleotide sequence following the 3′ end of the guide sequence:

(SEQ ID NO: 4)
GUUUUAGAGCUAGAAAUAGCAAGUUAAAAUAAGGCUAGUCCGUUAUCAA
CUUGAAAAAGUGGCACCGAGUCGGUGCUUUU in 5′ to 3′
orientation;
or
(SEQ ID NO: 26)
GUUUUAGAGCUAGAAAUAGCAAGUUAAAAUAAGGCUAGUCCGUUAUCAA
AAUGGCACCGAGUCGGUGCU in 5′ to 3′ orientation.

In the case of a sgRNA, the guide sequences may be integrated into the following modified motif: mN*mN*mN*NNNNNNNNNNNNNNNNNGUUUUAGAmGmCmUmAmGmAmAmAmUmAmGmC AAGUUAAAAUAAGGCUAGUCCGUUAUCAmAmCmUmUmGmAmAmAmAmAmGmUmGmGmC mAmCmCmGmAmGmUmCmGmGmUmGmCmU*mU*mU*mU (SEQ ID NO: 309) where “N” may be any natural or non-natural nucleotide, preferably an RNA nucleotide; sugar moieties of the nucleotide can be ribose, deoxyribose, or similar compounds with substitutions; m is a 2′-O-methyl modified nucleotide, and * is a phosphorothioate linkage to the adjacent nucleotide residue; and wherein the N's are collectively the nucleotide sequence of a guide sequence. In the context of a modified sequence, unless otherwise indicated, A, C, G, and U are an unmodified adenine, cytosine, guanine, and uridine, respectively, RNA nucleotides, i.e., a 2′-OH sugar moiety with a phosphodiesterase linkage to the adjacent nucleotide residue, or a 5′-terminal PO4; and N is any of A, C, G, and U.

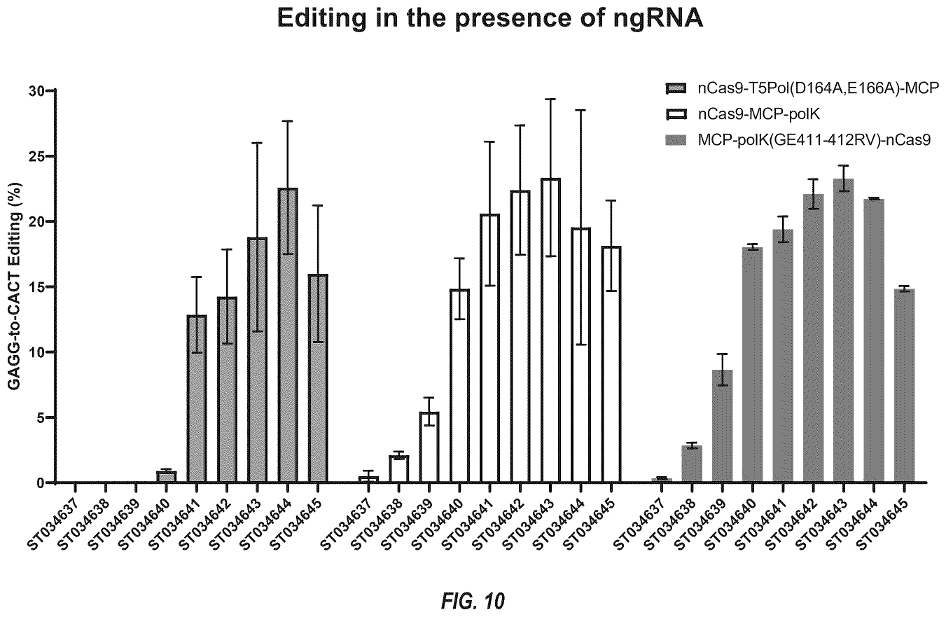

In the case of an sgRNA, the guide sequences may further comprise a SpyCas9 sgRNA scaffold sequence (also referred to herein as the conserved portion of a SpyCas9 sgRNA sequence) disclosed herein (See e.g., Tables 1A and 1C). Thus, in some embodiments, the sgRNA may comprise, from 5′ to 3′, the guide sequence and the conserved portion of the SpyCas9 sgRNA sequence described herein. An example of a SpyCas9 sgRNA sequence is shown in Table 5 (SEQ ID NO: 22: GUUUUAGAGCUAGAAAUAGCAAGUUAAAAUAAGGCUAGUCCGUUAUCAACUUGAAAAAG UGGCACCGAGUCGGUGC-“Exemplary SpyCas9 sgRNA-1”), included at the 3′ end of the guide sequence, and provided with the domains as shown in Table 5A below. LS is lower stem. B is bulge. US is upper stem. H1 and H2 are hairpin 1 and hairpin 2, respectively. Collectively H1 and H2 are referred to as the hairpin region. A model of the structure is provided in FIG. 10A of WO2019237069 which is incorporated herein by reference.

The nucleotide sequence of Exemplary SpyCas9 sgRNA-1 may serve as a template or a backbone sequence for specific chemical modifications, sequence substitutions and truncations.

In certain embodiments, the guide RNA is an sgRNA or a dgRNA, for example, and it optionally comprises a chemical modification. In some embodiments, the modified sgRNA comprises a guide sequence and a SpyCas9 sgRNA sequence, e.g., Exemplary SpyCas9 sgRNA-1. A guide RNA, such as an sgRNA, may include modifications on the 5′ end of the guide sequence or on the 3′ end of the SpyCas9 sgRNA sequence, such as, e.g., Exemplary SpyCas9 sgRNA-1 at one or more of the terminal nucleotides, e.g., at 1, 2, 3, or 4 of the nucleotides at the 3′ end or at the 5′ end. In certain embodiments, the modified nucleotide is selected from a 2′-O-methyl (2′-Ome or 2′-O-Me) modified nucleotide, a 2′-O-(2-methoxyethyl) (2′-O-moe) modified nucleotide, a 2′-fluoro (2′-F) modified nucleotide, a phosphorothioate (PS) linkage between nucleotides, or an inverted abasic modified nucleotide; or a combination thereof. In certain embodiments, the modified nucleotide includes a 2′-Ome modified nucleotide. In certain embodiments, the modified nucleotide includes a PS linkage. In certain embodiments, the modified nucleotide includes a 2′-Ome modified nucleotide and a PS linkage.

In certain embodiments, using SEQ ID NO: 22 (“Exemplary SpyCas9 sgRNA-1”) as an example, the Exemplary SpyCas9 sgRNA-1 further includes one or more of: (A) a shortened hairpin 1 region, or a substituted and optionally shortened hairpin 1 region, wherein (1) at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks (a) any one or two of H1-5 through H1-8, (b) one, two, or three of the following pairs of nucleotides: H1-1 and H1-12, H1-2 and H1-11, H1-3 and H1-10, and H1-4 and H1-9, or (c) 1-8 nucleotides of hairpin 1 region; or (2) the shortened hairpin 1 region lacks 4-8 nucleotides, preferably 4-6 nucleotides, and (a) one or more of positions H1-1, H1-2, or H1-3 is deleted or substituted relative to Exemplary SpyCas9 sgRNA-1 (SEQ ID NO: 22), or (b) one or more of positions H1-6 through H1-10 is substituted relative to Exemplary SpyCas9 sgRNA-1 (SEQ ID NO: 22); or (3) the shortened hairpin 1 region lacks 5-10 nucleotides, preferably 5-6 nucleotides, and one or more of positions N18, H1-12, or n is substituted relative to Exemplary SpyCas9 sgRNA-1 (SEQ ID NO: 22); or (B) a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1 (SEQ ID NO: 22); or (C) a substitution relative to Exemplary SpyCas9 sgRNA-1 (SEQ ID NO: 22) at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or (D) an Exemplary SpyCas9 sgRNA-1 (SEQ ID NO: 22) with an upper stem region, wherein the upper stem modification comprises a modification to any one or more of US1-US12 in the upper stem region, wherein (1) the modified nucleotide is optionally selected from a 2′-O-methyl (2′-Ome) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof; or (2) the modified nucleotide optionally includes a 2′-Ome modified nucleotide.

In some embodiments, the unmodified sgRNA comprises the following sequence:

(SEQ ID NO: 176 or 120)
(N)₂₀GUUUUAGAGCUAGAAAUAGCAAGUUAAAAUAAGGCUAGUCCGUUA
UCACGAAAGGGCACCGAGUCGGUGC;
or
(SEQ ID NO: 105)
(N)₂₀GUUUUAGAGCUAGAAAUAGCAAGUUAAAAUAAGGCUAGUCCGUUA
UCACGAAAGGGCACCGAGUCGGUGCU.

In some embodiments, the sgRNA comprises a modified motif disclosed herein, including any modified motif shown in Tables 1A, 1B, 1C, and 1D, where a guide RNA, or “N” may be any natural or non-natural nucleotide, preferably an RNA nucleotide; sugar moieties of the nucleotide can be ribose, deoxyribose, or similar compounds with substitutions; m is a 2′-O-methyl modified nucleotide, and * is a phosphorothioate linkage to the adjacent nucleotide residue; and wherein the N's are collectively the nucleotide sequence of a guide sequence.

In the context of a modified sequence, unless otherwise indicated, A, C, G, and U are an unmodified adenine, cytosine, guanine, and uridine, respectively, RNA nucleotide, i.e., a 2′-OH sugar moiety with a phosphodiester linkage to the adjacent nucleotide residue, or a 5′-terminal PO4; and N is any of A, C, G, and U.

In some embodiments, the guide RNA that directs the SpyCas9 nickase to a genomic locus is a SpyCas9 guide RNA. In some embodiments, the SpyCas9 guide RNA is a single guide RNA comprising: a conserved portion of an sgRNA comprising an upper stem and hairpin region, wherein every nucleotide in the upper stem region is modified with 2′-O-Me, and every nucleotide in the hairpin region is modified with 2′-O-Me; a 3′ end modification comprising 2′-O-Me modified nucleotides at the last three nucleotides of the 3′ end and phosphorothioate (PS) bonds between the last four nucleotides of the 3′ end; and 5′ end modification comprising 2′-O-Me modified nucleotides at the first three nucleotides of the 5′ end; and phosphorothioate (PS) bonds between the first four nucleotides of the 5′ end.

In some embodiments, the SpyCas9 guide RNA is a short-single guide RNA (short-sgRNA) comprising a conserved portion of an sgRNA comprising a hairpin region, wherein the hairpin region lacks at least 5-10 nucleotides and wherein the short-sgRNA comprises (i) a 5′ end modification or (ii) a 3′ end modification.

In some embodiments, the first guide RNA is a SpyCas9 guide RNA that is a single guide RNA comprising a nucleotide sequence selected from SEQ ID NOs: 159-167, 170-177, and 180-194, or a nucleotide sequence that is at least 85%, 90%, or 95% identical to SEQ ID NOs: 159-167, 170-177, and 180-194.

In some embodiments, the first guide RNA is a SpyCas9 guide RNA that is a single guide RNA comprising a nucleotide sequence selected from SEQ ID NOs: 1-39 or 101-130, or a nucleotide sequence that is at least 85%, 90%, or 95% identical to SEQ ID NOs: 1-39 or 101-130. In some embodiments, the first guide RNA is a SpyCas9 guide RNA that is a single guide RNA comprising a nucleotide sequence of SEQ ID NO: 27.

In some embodiments, the sgRNA comprises Exemplary SpyCas9 sgRNA-1 or the modified versions thereof provided herein, or a version as provided in Tables 1B or 1D, where the totality of the N's comprise a guide sequence that directs a SpyCas9 nickase to a target sequence. Each N is independently modified or unmodified. In certain embodiments, in the absence of an indication of a modification, the nucleotide is an unmodified RNA nucleotide residue, i.e., a ribose sugar and a phosphodiester backbone.

TABLE 1A
Exemplary Unmodified SpyCas9 Scaffold Sequences

| | | SEQ ID |
|---|---|---|
| #mer | Unmodified nucleotide sequence | NO |
| 75 | GUUUUAGAAAGUUAAAAUAAGGCUAGUCCGUUAUCACGGGCACCGAGUCGGUGCU | 1 |
| 79 | GUUUUAGAGCGCAAGUUAAAAUAAGGCUAGUCCGUUAUCACGGGCACCGAGUCGGUGCU | 2 |
| 83 | GUUUUAGAGCUAUAGCAAGUUAAAAUAAGGCUAGUCCGUUAUCACGGGCACCGAGUCGG | 3 |
| | UGCU | |
| 100 | GUUUUAGAGCUAGAAAUAGCAAGUUAAAAUAAGGCUAGUCCGUUAUCAACUUGAAAAAG | 4 |
| | UGGCACCGAGUCGGUGCUUUU | |
| 91 | GUUUUAGAGCUAGAAAUAGCAAGUUAAAAUAAGGCUAGUCCGUUAUCACGAAAGGGCAC | 5 |
| | CGAGUCGGUGCU | |
| 83 | GUUUUAGAGUAUAGCAAGUUAAAAUAAGGCUAGUCCGUUAUCACGGGCACCGAGUCGGU | 6 |
| | GCU | |
| 79 | GUUUUAGAGGCAAGUUAAAAUAAGGCUAGUCCGUUAUCACGGGCACCGAGUCGGUGCU | 7 |
| 84 | GUUUUAGAGCUAUAGCAAGUUAAAAUAAGGCGUCCGUUAUCACGGGCACCGAGUCGGUG | 8 |
| | CU | |
| 42 | GUUUUAGAGCUAUGCUGUUUGG | 9 |
| 39 | GUUUUAGAGCUAUGCUGUG | 10 |
| 36 | GUUUUAGAGCUAUGCG | 11 |
| 33 | GUUUUAGAGCUAG | 12 |
| 43 | GUUUUAGAGUAUGCUGUUUGGU | 13 |
| 43 | GUUUUAGAGUAUGUGUUUGGU | 14 |
| 66 | ACCAAACAGCAUAGCAAGUUAAAAUAAGGCUAGUCCGUUAUCACGAAAGGGCACCGAGU | 15 |
| | CGGUGCU | |
| 65 | CCAAACAGCAUAGCAAGUUAAAAUAAGGCUAGUCCGUUAUCACGAAAGGGCACCGAGUC | 16 |
| | GGUGCU | |
| 62 | CACAGCAUAGCAAGUUAAAAUAAGGCUAGUCCGUUAUCACGAAAGGGCACCGAGUCGGU | 17 |
| | GCU | |
| 59 | CGCAUAGCAAGUUAAAAUAAGGCUAGUCCGUUAUCACGAAAGGGCACCGAGUCGGUGCU | 18 |
| 56 | CUAGCAAGUUAAAAUAAGGCUAGUCCGUUAUCACGAAAGGGCACCGAGUCGGUGCU | 19 |
| 61 | CCAAACAGCAUAGCAAGUUAAAAUAAGGCUAGUCCGUUAUCACGGGCACCGAGUCGGUG | 20 |
| | CU | |
| | GUUUUAGAGCUAUGCUGUUUUG | 21 |
| 96 | GUUUUAGAGCUAGAAAUAGCAAGUUAAAAUAAGGCUAGUCCGUUAUCAACUUGAAAAAG | 22 |
| | UGGCACCGAGUCGGUGC | |
| 97 | GUUUUAGAGCUAGAAAUAGCAAGUUAAAAUAAGGCUAGUCCGUUAUCAACUUGAAAAAG | 23 |
| | UGGCACCGAGUCGGUGCU | |
| 88 | GUUUUAGAGCUAGAAAUAGCAAGUUAAAAUAAGGCUAGUCCGUUAUCAACUUGGCACCG | 24 |
| | AGUCGGUGC | |
| 88 | GUUUUAGAGCUAGAAAUAGCAAGUUAAAAUAAGGCUAGUCCGUUAUCAAAAUGGCACCG | 25 |
| | AGUCGGUGC | |
| 89 | GUUUUAGAGCUAGAAAUAGCAAGUUAAAAUAAGGCUAGUCCGUUAUCAAAAUGGCACCG | 26 |
| | AGUCGGUGCU | |
| 90 | GUUUUAGAGCUAGAAAUAGCAAGUUAAAAUAAGGCUAGUCCGUUAUCACGAAAGGGCAC | 27 |
| | CGAGUCGGUGC | |
| 88 | GGUUUUAGAGCUAGAAAUAGCAAGUUAAAAUAAGGCUAGUCCGUUAUCAAAAUGGCACC | 28 |
| | GAGUCGGUGC | |
| 108 | GUUUUAGAGCGCAAGUUAAAAUAAGGCUAGUCCGUUAUCACGGGCACCGAGUCGGUGCU | 29 |
| | GCCAUCUGGAGCAGUGAUCUGGCCUGCAG | |
| 109 | GUUUUAGAGCGCAAGUUAAAAUAAGGCUAGUCCGUUAUCACGGGCACCGAGUCGGUGCU | 30 |
| | GCCAUCUGGAGCAGUGAUCUGGCCUGCAGU | |
| 110 | GUUUUAGAGCGCAAGUUAAAAUAAGGCUAGUCCGUUAUCACGGGCACCGAGUCGGUGCU | 31 |
| | GCCAUCUGGAGCAGUGAUCUGGCCUGCAGUU | |
| 111 | GUUUUAGAGCGCAAGUUAAAAUAAGGCUAGUCCGUUAUCACGGGCACCGAGUCGGUGCU | 32 |
| | GCCAUCUGGAGCAGUGAUCUGGCCUGCAGUUU | |
| 110 | GUUUUAGAGCUAUAGCAAGUUAAAAUAAGGCUAGUCCGUUAUCACGGGCACCGAGUCGG | 33 |
| | UGCUGCCAUCUGGAGCAGUGAUCUGGCCUGC | |
| 112 | GUUUUAGAGCUAUAGCAAGUUAAAAUAAGGCUAGUCCGUUAUCACGGGCACCGAGUCGG | 34 |
| | UGCUGCCAUCUGGAGCAGUGAUCUGGCCUGCAG | |
| 91 | GUUUUAGAGCUAGAAAUAGCAAGUUAAAAUAAGGCUAGUCCGUUAUCACGAAAGGGCAC | 35 |
| | CGAGUCGGUGCU | |
| 105 | GUUUUAGAGCGCAAGUUAAAAUAAGGCUAGUCCGUUAUCACGGGCACCGAGUCGGUGCA | 36 |
| | AAUAGACACGUAAUCUUACUGGAAGU | |
| 105 | GUUUUAGAGCUAUAGCAAGUUAAAAUAAGGCUAGUCCGUUAUCACGGGCACCGAGUCGG | 37 |
| | UGCGCAAGAUGGCAGCGCGAACAGCU | |
| 78 | GUUUUAGAGCGCAAGUUAAAAUAAGGCUAGUCCGUUAUCACGGGCACCGAGUCGGUGC | 38 |
| 82 | GUUUUAGAGCUAUAGCAAGUUAAAAUAAGGCUAGUCCGUUAUCACGGGCACCGAGUCGG | 39 |
| | UGC | |
| 90 | GUUUUAGACGUAGAAAUACGAAGUUAAAAUAAGGCUAGUCCGUUAUCACGAAAGGGCAC | 40 |
| | CGAGUCGGUGC | |

In some embodiments, the guide RNA comprises a nucleotide sequence selected from the SpyCas9 guide RNA sequences in Table 1B, wherein the (N) 20's are collectively a guide sequence. In some embodiments, each nucleotide of the Spy guide RNA Sequences in Table 1B is any natural or nonnatural nucleotide. In some embodiments, the guide RNA comprises a nucleotide sequence of SEQ ID NO: 120.

TABLE 1B
Exemplary Unmodified SpyCas9 Guide RNA Sequences

| | | SEQ ID |
|---|---|---|
| #mer | Unmodified nucleotide sequence | NO |
| 75 | (N)20GUUUUAGAAAGUUAAAAUAAGGCUAGUCCGUUAUCACGGGCACCGAGUCGGUG | 101 |
| | CU | |
| 79 | (N)20GUUUUAGAGCGCAAGUUAAAAUAAGGCUAGUCCGUUAUCACGGGCACCGAGUC | 102 |
| | GGUGCU | |
| 83 | (N)20GUUUUAGAGCUAUAGCAAGUUAAAAUAAGGCUAGUCCGUUAUCACGGGCACCG | 103 |
| | AGUCGGUGCU | |
| 100 | (N)20GUUUUAGAGCUAGAAAUAGCAAGUUAAAAUAAGGCUAGUCCGUUAUCAACUUG | 104 |
| | AAAAAGUGGCACCGAGUCGGUGCUUUU | |
| 91 | (N)20GUUUUAGAGCUAGAAAUAGCAAGUUAAAAUAAGGCUAGUCCGUUAUCACGAAA | 105 |
| | GGGCACCGAGUCGGUGCU | |
| 83 | (N)20GUUUUAGAGUAUAGCAAGUUAAAAUAAGGCUAGUCCGUUAUCACGGGCACCGA | 106 |
| | GUCGGUGCU | |
| 79 | (N)20GUUUUAGAGGCAAGUUAAAAUAAGGCUAGUCCGUUAUCACGGGCACCGAGUCG | 107 |
| | GUGCU | |
| 84 | (N)20GUUUUAGAGCUAUAGCAAGUUAAAAUAAGGCGUCCGUUAUCACGGGCACCGAG | 108 |
| | UCGGUGCU | |
| 42 | (N)20GUUUUAGAGCUAUGCUGUUUGG | 109 |
| 39 | (N)20GUUUUAGAGCUAUGCUGUG | 110 |
| 36 | (N)20GUUUUAGAGCUAUGCG | 111 |
| 33 | (N)20GUUUUAGAGCUAG | 112 |
| 43 | (N)20GUUUUAGAGUAUGCUGUUUGGU | 113 |
| 43 | (N)20GUUUUAGAGUAUGUGUUUGGU | 114 |
| 96 | (N)20GUUUUAGAGCUAGAAAUAGCAAGUUAAAAUAAGGCUAGUCCGUUAUCAACUUG | 115 |
| | AAAAAGUGGCACCGAGUCGGUGC | |
| 97 | (N)20GUUUUAGAGCUAGAAAUAGCAAGUUAAAAUAAGGCUAGUCCGUUAUCAACUUG | 116 |
| | AAAAAGUGGCACCGAGUCGGUGCU | |
| 88 | (N)20GUUUUAGAGCUAGAAAUAGCAAGUUAAAAUAAGGCUAGUCCGUUAUCAACUUG | 117 |
| | GCACCGAGUCGGUGC | |
| 88 | (N)20GUUUUAGAGCUAGAAAUAGCAAGUUAAAAUAAGGCUAGUCCGUUAUCAAAAUG | 118 |
| | GCACCGAGUCGGUGC | |
| 88 | (N)20GGUUUUAGAGCUAGAAAUAGCAAGUUAAAAUAAGGCUAGUCCGUUAUCAAAAU | 119 |
| | GGCACCGAGUCGGUGC | |
| 90 | (N)20GUUUUAGAGCUAGAAAUAGCAAGUUAAAAUAAGGCUAGUCCGUUAUCACGAAA | 120 |
| | GGGCACCGAGUCGGUGC | |
| 108 | (N)20GUUUUAGAGCGCAAGUUAAAAUAAGGCUAGUCCGUUAUCACGGGCACCGAGUC | 121 |
| | GGUGCUGCCAUCUGGAGCAGUGAUCUGGCCUGCAG | |
| 109 | (N)20GUUUUAGAGCGCAAGUUAAAAUAAGGCUAGUCCGUUAUCACGGGCACCGAGUC | 122 |
| | GGUGCUGCCAUCUGGAGCAGUGAUCUGGCCUGCAGU | |
| 110 | (N)20GUUUUAGAGCGCAAGUUAAAAUAAGGCUAGUCCGUUAUCACGGGCACCGAGUC | 123 |
| | GGUGCUGCCAUCUGGAGCAGUGAUCUGGCCUGCAGUU | |
| 111 | (N)20GUUUUAGAGCGCAAGUUAAAAUAAGGCUAGUCCGUUAUCACGGGCACCGAGUC | 124 |
| | GGUGCUGCCAUCUGGAGCAGUGAUCUGGCCUGCAGUUU | |
| 110 | (N)20GUUUUAGAGCUAUAGCAAGUUAAAAUAAGGCUAGUCCGUUAUCACGGGCACCG | 125 |
| | AGUCGGUGCUGCCAUCUGGAGCAGUGAUCUGGCCUGC | |
| 112 | (N)20GUUUUAGAGCUAUAGCAAGUUAAAAUAAGGCUAGUCCGUUAUCACGGGCACCG | 126 |
| | AGUCGGUGCUGCCAUCUGGAGCAGUGAUCUGGCCUGCAG | |
| 105 | (N)20GUUUUAGAGCGCAAGUUAAAAUAAGGCUAGUCCGUUAUCACGGGCACCGAGUC | 127 |
| | GGUGCAAAUAGACACGUAAUCUUACUGGAAGU | |
| 105 | (N)20GUUUUAGAGCUAUAGCAAGUUAAAAUAAGGCUAGUCCGUUAUCACGGGCACCG | 128 |
| | AGUCGGUGCGCAAGAUGGCAGCGCGAACAGCU | |
| 78 | (N)20GUUUUAGAGCGCAAGUUAAAAUAAGGCUAGUCCGUUAUCACGGGCACCGAGUC | 129 |
| | GGUGC | |
| 82 | (N)20GUUUUAGAGCUAUAGCAAGUUAAAAUAAGGCUAGUCCGUUAUCACGGGCACCG | 130 |
| | AGUCGGUGC | |
| 90 | (N)20GUUUUAGACGUAGAAAUACGAAGUUAAAAUAAGGCUAGUCCGUUAUCACGAAA | 131 |
| | GGGCACCGAGUCGGUGC | |

wherein the Ns collectively are a guide sequence provided herein. Within the table, in the context of an unmodified sequence, A, C, G, U, and N are, independently, any natural or non-natural adenine, cytosine, guanine, uridine, and any nucleotide (e.g., A, C, G, or U), respectively.

In some embodiments of the SpyCas9 guide RNA disclosed herein, the guide sequence may be integrated into the following modified guide scaffold (Table 1C). The #mer refers to the length of the guide RNA when a 20-nucleotide guide sequence, either a modified or unmodified sequence, is included 5′ to the scaffold sequence provided in Table 1C:

TABLE 1C
Exemplary Modified Spy Guide Scaffold Sequences

| | | SEQ ID |
|---|---|---|
| #mer | Modified sequence | NO |
| 75 | GUUUUAGAdSAAGUUAAAAUAAGGCUAGUCCGUUAUCAC(L1)GGGCACCGAGUCGGmU | 201 |
| | *mG*mC*mU | |
| 75 | GUUUUAGA(L4)AAGUUAAAAUAAGGCUAGUCCGUUAUCAC(L1)GGGCACCGAGUCGG | 202 |
| | mU*mG*mC*mU | |
| 75 | GUUUUAGA(L3)AAGUUAAAAUAAGGCUAGUCCGUUAUCAC(L1)GGGCACCGAGUCGG | 203 |
| | mU*mG*mC*mU | |
| 75 | GUUUUAGA(L2)AAGUUAAAAUAAGGCUAGUCCGUUAUCAC(L1)GGGCACCGAGUCGG | 204 |
| | mU*mG*mC*mU | |
| 75 | GUUUUAGA(L1)AAGUUAAAAUAAGGCUAGUCCGUUAUCAC(L1)GGGCACCGAGUCGG | 205 |
| | mU*mG*mC*mU | |
| 79 | GUUUUAGAmGmC(L1)mGmCAAGUUAAAAUAAGGCUAGUCCGUUAUCAC(L1)GGGCAC | 206 |
| | CGAGUCGGmU*mG*mC*mU | |
| 83 | GUUUUAGAmGmCmUmA(L1)mUmAmGmCAAGUUAAAAUAAGGCUAGUCCGUUAUCAC | 207 |
| | (L1)GGGCACCGAGUCGGmU*mG*mC*mU | |
| 83 | GUUUUAGAmGmCmUmA(L1)mUmAmGmCAAGUUAAAAUAAGGCUAGUCCGUUAUCAC | 208 |
| | (L1)GGGCACCGAGUmCmGmGmU*mG*mC*mU | |
| 100 | GUUUUAGAmGmCmUmAmGmAmAmAmUmAmGmCAAGUUAAAAUAAGGCUAGUCCGUUAUC | 209 |
| | AmAmCmUmUmGmAmAmAmAmAmGmUmGmGmCmAmCmCmGmAmGmUmCmGmGmUmGmCmU | |
| | *mU*mU*mU | |
| 91 | GUUUUAGAmGmCmUmAmGmAmAmAmUmAmGmCAAGUUAAAAUAAGGCUAGUCCGUUAUC | 210 |
| | ACGAAAGGGCACCGAGUCGGmU*mG*mC*mU | |
| 90 | GUUUUAGAmGmCmUmA(L1)mUmAmGmCAAGUUAAAAUAAGGCUAGUCCGUUAUCAC | 211 |
| | (L1)GGGCACCGAGUCGGmUmGmC | |
| 90 | GUUUUAGAmGmCmUmAmGmAmAmAmUmAmGmCAAGUUAAAAUAAGGCUAGUCCGUUAUC | 212 |
| | ACGAAAGGGCACCGAGUCGGmUmGmC | |
| 84 | GUUUUAGAmGmCmUmA(L1)mUmAmGmCAAGUUAAAAUAAGGC(L2)GUCCGUUAUCAC | 213 |
| | (L1)GGGCACCGAGUCGGmUmGmC*mU | |
| 42 | GUUUUAGAmGmCmUmAmUmGmCmUmGmUmUmUmG*mG | 214 |
| 42 | GUUUUAGAmGmCmUmAmUmGmCmUmGmUmUmU*mG*mG | 215 |
| 39 | GUUUUAGAmGmCmUmAmUmGmCmUmG*mU*mG | 216 |
| 36 | GUUUUAGAmGmCmUmAmUmG*mC*mG | 217 |
| 33 | GUUUUAGAmGmCmU*mA*mG | 218 |
| | mGUUUUAGmAmGmCmUmAmGmAmAmAmUmAmGmCmAAGUUmAAmAAmUmAmAGmGCUmA | 223 |
| | GUCmCmGUUAUmCAmCGmAmAmAmGmGmCmAmCmCmGmAGUCmGmGmUmGmC | |
| | mGUUUUAGmAmGmCmUmA(L1)mUmAmGmCmAAGUUmAAmAAmUmAmAGmGCUmAGUCm | 224 |
| | CmGUUAUmCAmC(L1)mGGmGmCmAmCmCmGmAGUCmGmGmUmGmC | |
| 42 | GUUUUAGAmGmCmUmA+T+G+C+T+G+T+T+T+G+G | 225 |
| 42 | GUUUUAGAmGmCmUmAmUmGmCmUmG+T+T+T+G+G | 226 |
| 66 | mA*mC*mC*mAmAmAmCmAmGmCmAmUmAmGmCAAGUUAAAAUAAGGCUAGUCCGUUAU | 227 |
| | CACGAAAGGGCACCGAGUCGGmU*mG*mC*mU | |
| 65 | mC*mC*mA*mAmAmCmAmGmCmAmUmAmGmCAAGUUAAAAUAAGGCUAGUCCGUUAUCA | 228 |
| | CGAAAGGGCACCGAGUCGGmU*mG*mC*mU | |
| 62 | mC*mA*mC*mAmGmCmAmUmAmGmCAAGUUAAAAUAAGGCUAGUCCGUUAUCACGAAAG | 229 |
| | GGCACCGAGUCGGmU*mG*mC*mU | |
| 59 | mC*mG*mC*mAmUmAmGmCAAGUUAAAAUAAGGCUAGUCCGUUAUCACGAAAGGGCACC | 230 |
| | GAGUCGGmU*mG*mC*mU | |
| 56 | mC*mU*mA*mGmCAAGUUAAAAUAAGGCUAGUCCGUUAUCACGAAAGGGCACCGAGUCG | 231 |
| | GmU*mG*mC*mU | |
| 58 | GUUUUAGAmGmC(L1)mGmCAAGUUAAAAUAAGGCUAGUCCGUUAUCAC(L1)GGGCAC | 232 |
| | CGAGUCGGmUmGmC | |
| 54 | GUUUUAGAdSAAGUUAAAAUAAGGCUAGUCCGUUAUCAC(L1)GGGCACCGAGUCGGmU | 233 |
| | mGmC | |
| 57 | GUUUUAGA(L4)AAGUUAAAAUAAGGCUAGUCCGUUAUCAC(L1)GGGCACCGAGUCGG | 234 |
| | mUmGmC | |
| 57 | GUUUUAGA(L3)AAGUUAAAAUAAGGCUAGUCCGUUAUCAC(L1)GGGCACCGAGUCGG | 235 |
| | mUmGmC | |
| 61 | mC*mC*mA*mAmAmCmAmGmCmAmUmAmGmCAAGUUAAAAUAAGGCUAGUCCGUUAUCA | 236 |
| | C(L1)GGGCACCGAGUCGGmUmG*mC*mU | |
| 54 | GUUUUAGA(L2)AAGUUAAAAUAAGGCUAGUCCGUUAUCAC(L1)GGGCACCGAGUCGG | 237 |
| | mUmGmC | |
| 54 | GUUUUAGA(L1)AAGUUAAAAUAAGGCUAGUCCGUUAUCAC(L1)GGGCACCGAGUCGG | 238 |
| | mUmGmC | |
| 62 | GUUUUAGAmGmCmUmA(L1)mUmAmGmCAAGUUAAAAUAAGGCUAGUCCGUUAUCAC | 239 |
| | (L1)GGGCACCGAGUmCmGmGmUmGmC | |
| 100 | GUUUUAGAmGmCmUmAmGmAmAmAmUmAmGmCAAGUUAAAAUAAGGCUAGUCCGUUAUC | 240 |
| | AmAmCmUmUmGmAmAmAmAmAmGmUmGmGmCmAmCmCmGmAmGmUmCmGmGmUmGmCmU | |
| | *mU*mU*mU | |
| 96 | GUUUUAGAmGmCmUmAmGmAmAmAmUmAmGmCAAGUUAAAAUAAGGCUAGUCCGUUAUC | 241 |
| | AmAmCmUmUmGmAmAmAmAmAmGmUmGmGmCmAmCmCmGmAmGmUmCmGmGmUmGmC | |
| 97 | GUUUUAGAmGmCmUmAmGmAmAmAmUmAmGmCAAGUUAAAAUAAGGCUAGUCCGUUAUC | 242 |
| | AmAmCmUmUmGmAmAmAmAmAmGmUmGmGmCmAmCmCmGmAmGmUmCmGmGmUmGmCmU | |
| 88 | GUUUUAGAmGmCmUmAmGmAmAmAmUmAmGmCAAGUUAAAAUAAGGCUAGUCCGUUAUC | 243 |
| | AACUUGGCACCGAGUCGG*mU*mG*mC | |
| 88 | GUUUUAGAmGmCmUmAmGmAmAmAmUmAmGmCAAGUUAAAAUAAGGCUAGUCCGUUAUC | 244 |
| | AAAAUGGCACCGAGUCGG*mU*mG*mC | |
| 88 | GUUUUAGAmGmCmUmAmGmAmAmAmUmAmGmCAAGUUAAAAUAAGGCUAGUCCGUUAUC | 245 |
| | AmAmAmAmUmGmGmCmAmCmCmGmAmGmUmCmGmG*mU*mG*mC | |
| 90 | GUUUUAGAmGmCmUmAmGmAmAmAmUmAmGmCAAGUUAAAAUAAGGCUAGUCCGUUAUC | 246 |
| | ACGAAAGGGCACCGAGUCGG*mU*mG*mC | |
| 90 | GUUUUAGAmGmCmUmAmGmAmAmAmUmAmGmCAAGUUAAAAUAAGGCUAGUCCGUUAUC | 247 |
| | AmCmGmAmAmAmGmGmGmCmAmCmCmGmAmGmUmCmGmG*mU*mG*mC | |
| 91 | GUUUUAGAmGmCmUmAmGmAmAmAmUmAmGmCAAGUUAAAAUAAGGCUAGUCCGUUAUC | 248 |
| | ACGAAAGGGCACCGAGUCGGU*mG*mC*mU | |
| 91 | GUUUUAGAmGmCmUmAmGmAmAmAmUmAmGmCAAGUUAAAAUAAGGCUAGUCCGUUAUC | 249 |
| | ACGAAAGGGCACCGAGUCGGmUmGmC*mU | |
| 91 | GUUUfUAGmAmGmCmUmAmGmAmAmAmUmAmGmCmAmAGUfUmAfAmAfAmUAmAmGmG | 250 |
| | mCmUmAGUmCmCGUfUAmUmCAmCmGmAmAmAmGmGmGmCmAmCmCmGmAmGmUmCmGm | |
| | GmU*mG*mC*mU | |
| 108 | GUUUUAGAmGmC(L1)mGmCAAGUUAAAAUAAGGCUAGUCCGUUAUCAC(L1)GGGCAC | 251 |
| | CGAGUCGGmUmGmCdTdGdCdCdAdTdCdTdGdGdAdGdCdAdGdTdGdAdTCUGGmCm | |
| | CmUmGmCmA*mG | |
| 108 | GUUUUAGAmGmC(L1)mGmCAAGUUAAAAUAAGGCUAGUCCGUUAUCAC(L1)GGGCAC | 252 |
| | CGAGUCGGmUmGmCdTdGdCdCdAdTdCdTdGdGdAdGdCdAdGdTdGdAdTCUGGmCm | |
| | CmUmGmC*mA*mG | |
| 108 | GUUUUAGAmGmC(L1)mGmCAAGUUAAAAUAAGGCUAGUCCGUUAUCAC(L1)GGGCAC | 253 |
| | CGAGUCGGmUmGmCdTdGdCdCdAdTdCdTdGdGdAdGdCdAdGdTdGdAdTCUGGmCm | |
| | CmUmG*mC*mA*mG | |
| 108 | GUUUUAGAmGmC(L1)mGmCAAGUUAAAAUAAGGCUAGUCCGUUAUCAC(L1)GGGCAC | 254 |
| | CGAGUCGGmUmGmCdTdGdCdCdAdTdCdTdGdGdAdGdCdAdGdTdGdAdTCUGmGmC | |
| | mCmUmG*mC*mA*mG | |
| 108 | GUUUUAGAmGmC(L1)mGmCAAGUUAAAAUAAGGCUAGUCCGUUAUCAC(L1)GGGCAC | 255 |
| | CGAGUCGGmUmGmCdTdGdCdCdAdTdCdTdGdGdAdGdCdAdGdTdGdAdTCUmGmGm | |
| | CmCmUmG*mC*mA*mG | |
| 108 | GUUUUAGAmGmC(L1)mGmCAAGUUAAAAUAAGGCUAGUCCGUUAUCAC(L1)GGGCAC | 256 |
| | CGAGUCGGmUmGmCdTdGdCdCdAdTdCdTdGdGdAdGdCdAdGdTdGdAdTCmUmGmG | |
| | mCmCmUmG*mC*mA*mG | |
| 108 | GUUUUAGAmGmC(L1)mGmCAAGUUAAAAUAAGGCUAGUCCGUUAUCAC(L1)GGGCAC | 257 |
| | CGAGUCGGmUmGmCdTdGdCdCdAdTdCdTdGdGdAdGdCdAdGdTdGdAdTmCmUmGm | |
| | GmCmCmUmG*mC*mA*mG | |
| 109 | GUUUUAGAmGmC(L1)mGmCAAGUUAAAAUAAGGCUAGUCCGUUAUCAC(L1)GGGCAC | 258 |
| | CGAGUCGGmUmGmCdTdGdCdCdAdTdCdTdGdGdAdGdCdAdGdTdGdAdTCUGGmCm | |
| | CmUmGmCmAmG*mU | |
| 110 | GUUUUAGAmGmC(L1)mGmCAAGUUAAAAUAAGGCUAGUCCGUUAUCAC(L1)GGGCAC | 259 |
| | CGAGUCGGmUmGmCdTdGdCdCdAdTdCdTdGdGdAdGdCdAdGdTdGdAdTCUGGmCm | |
| | CmUmGmCmAmGmU*mU | |
| 110 | GUUUUAGAmGmCmUmA(L1)mUmAmGmCAAGUUAAAAUAAGGCUAGUCCGUUAUCAC | 260 |
| | (L1)GGGCACCGAGUCGGmUmGmCdTdGdCdCdAdTdCdTdGdGdAdGdCdAdGdTdGdAd | |
| | TCUGGmCmCmUmG*mC | |
| 112 | GUUUUAGAmGmCmUmA(L1)mUmAmGmCAAGUUAAAAUAAGGCUAGUCCGUUAUCAC | 261 |
| | (L1)GGGCACCGAGUCGGmUmGmCdTdGdCdCdAdTdCdTdGdGdAdGdCdAdGUGAUCUG | |
| | GmCmCmUmGmCmA*mG | |
| 112 | GUUUUAGAmGmCmUmA(L1)mUmAmGmCAAGUUAAAAUAAGGCUAGUCCGUUAUCAC | 262 |
| | 1(L)GGGCACCGAGUCGGmUmGmCdTdGdCdCdAdTdCdTdGdGdAdGdCdAdGdTGAUCU | |
| | GGmCmCmUmGmCmA*mG | |
| 112 | GUUUUAGAmGmCmUmA(L1)mUmAmGmCAAGUUAAAAUAAGGCUAGUCCGUUAUCAC | 263 |
| | (L1)GGGCACCGAGUCGGmUmGmCdTdGdCdCdAdTdCdTdGdGdAdGdCdAdGdTdGAUC | |
| | UGGmCmCmUmGmCmA*mG | |
| 112 | GUUUUAGAmGmCmUmA(L1)mUmAmGmCAAGUUAAAAUAAGGCUAGUCCGUUAUCAC | 264 |
| | (L1)GGGCACCGAGUCGGmUmGmCdTdGdCdCdAdTdCdTdGdGdAdGdCdAdGdTdGdAU | |
| | CUGGmCmCmUmGmCmA*mG | |
| 112 | GUUUUAGAmGmCmUmA(L1)mUmAmGmCAAGUUAAAAUAAGGCUAGUCCGUUAUCAC | 265 |
| | (L1)GGGCACCGAGUCGGmUmGmCdTdGdCdCdAdTdCdTdGdGdAdGdCdAdGdTdGdAd | |
| | TdCUGGmCmCmUmGmCmA*mG | |
| 112 | GUUUUAGAmGmCmUmA(L1)mUmAmGmCAAGUUAAAAUAAGGCUAGUCCGUUAUCAC | 266 |
| | (L1)GGGCACCGAGUCGGmUmGmCdTdGdCdCdAdTdCdTdGdGdAdGdCdAdGdTdGdAd | |
| | TdCdTGGmCmCmUmGmCmA*mG | |
| 112 | GUUUUAGAmGmCmUmA(L1)mUmAmGmCAAGUUAAAAUAAGGCUAGUCCGUUAUCAC | 267 |
| | (L1)GGGCACCGAGUCGGmUmGmCdTdGdCdCdAdTdCdTdGdGdAdGdCdAdGdTdGdAd | |
| | TdCdTdGdGmCmCmUmGmCmA*mG | |
| 112 | GUUUUAGAmGmCmUmA(L1)mUmAmGmCAAGUUAAAAUAAGGCUAGUCCGUUAUCAC | 268 |
| | (L1)GGGCACCGAGUCGGmUmGmCdTdGdCdCdAdTdCdTdGdGdAdGdCdAdGdTdGdAd | |
| | TdCdTdGdGdCdCmUmGmCmA*mG | |
| 90 | GUUUUAGAmGmCmUmAmGmAmAmAmUmAmGmCAAGUUAAAAUAAGGCUAGUCCGUUAUC | 269 |
| | ACGAAAGGGCACCGAGUCGGmU*mG*mC | |
| 105 | mGUUUUAGmAmGmC(L1)mGmCmAAGUUmAAmAAmUmAmAGmGCUmAGUCmCmGUUAUm | 270 |
| | CAmC(L1)mGGmGmCmAmCmCmGmAGUCmGmGmUmGmCdAdAdAdTdAdGdAdCdAdCd | |
| | GdTdAdAdTdCUUACmUmGmGmAmAmG*mU | |
| 105 | GUUUUAGAmGmCmUmA(L1)mUmAmGmCAAGUUAAAAUAAGGCUAGUCCGUUAUCAC | 271 |
| | (L1)GGGCACCGAGUCGGmUmGmCdGdCdAdAdGdAdTdGdGdCdAdGdCdGdCGAACmAm | |
| | GmC*mU | |
| 78 | mGUUUUAGmAmGmC(L1)mGmCmAAGUUmAAmAAmUmAmAGmGCUmAGUCmCmGUUAUm | 272 |
| | CAmC(L1)mGGmGmCmAmCmCmGmAGUCmGmGmUmGmC | |
| 82 | GUUUUAGAmGmCmUmA(L1)mUmAmGmCAAGUUAAAAUAAGGCUAGUCCGUUAUCAC | |
| | (L1)GGGCACCGAGUCGGmUmGmC | 273 |
| | mGUUUUAGmAmGmCmUmAmGmAmAmAmUmAmGmCmAAGUUmAAmAAmUmAmAGmGCUmA | 274 |
| | GUCmCmGUUAUmCAmCGmAmAmAmGGmGmCmAmCmCmGmAGUCmGmGmU*mG*mC*mU | |
| | mGUUUUAGmAmGmCmUmAmGmAmAmAmUmAmGmCmAAGUUmAAmAAmUmAmAGmGCUmA | 275 |
| | GUCmCmGUUAUmCAmCGmAmAmAmGGmGmCmAmCmCmGmAGUCmGmGmUmGmC | |
| | mGUUUUAGmAmGmCmUmAmGmAmAmAmUmAmGmCmAAGUUmAAmAAmUmAmAGmGCUmA | 276 |
| | GUCmCmGUUAUmCAmCGmAmAmAmGGgcaccgaGUCggugc | |
| 111 | GUUUUAGAmGmC(L1)mGmCAAGUUAAAAUAAGGCUAGUCCGUUAUCAC(L1)GGGCAC | 300 |
| | CGAGUCGGmUmGmCdTdGdCdCdAdTdCdTdGdGdAdGdCdAdGdTdGdAdTCUGGmCm | |
| | CmUmGmCmAmGmUmU*mU | |

In some embodiments, the guide RNA comprises a nucleotide sequence selected from the SpyCas9 guide RNA sequences in Table 1D, wherein the mN*mN*mN*(N)17 or (mN*)3 N17 are collectively a guide sequence.

TABLE 1D
Exemplary Modified SpyCas9 Guide RNA Sequences

| | | SEQ ID |
|---|---|---|
| #mer | Modified sequence | NO |
| 75 | mN*mN*mN*(N)17GUUUUAGAdSAAGUUAAAAUAAGGCUAGUCCGUUAUCAC(L1)GG | 301 |
| | GCACCGAGUCGGmU*mG*mC*mU | |
| 75 | mN*mN*mN*(N)17GUUUUAGA(L4)AAGUUAAAAUAAGGCUAGUCCGUUAUCAC(L1) | 302 |
| | GGGCACCGAGUCGGmU*mG*mC*mU | |
| 75 | mN*mN*mN*(N)17GUUUUAGA(L3)AAGUUAAAAUAAGGCUAGUCCGUUAUCAC(L1) | 303 |
| | GGGCACCGAGUCGGmU*mG*mC*mU | |
| 75 | mN*mN*mN*(N)17GUUUUAGA(L2)AAGUUAAAAUAAGGCUAGUCCGUUAUCAC(L1) | 304 |
| | GGGCACCGAGUCGGmU*mG*mC*mU | |
| 75 | mN*mN*mN*(N)17GUUUUAGA(L1)AAGUUAAAAUAAGGCUAGUCCGUUAUCAC(L1) | 305 |
| | GGGCACCGAGUCGGmU*mG*mC*mU | |
| 79 | mN*mN*mN*(N)17GUUUUAGAmGmC(L1)mGmCAAGUUAAAAUAAGGCUAGUCCGUUA | 306 |
| | UCAC(L1)GGGCACCGAGUCGGmU*mG*mC*mU | |
| 83 | mN*mN*mN*(N)17GUUUUAGAmGmCmUmA(L1)mUmAmGmCAAGUUAAAAUAAGGCUA | 307 |
| | GUCCGUUAUCAC(L1)GGGCACCGAGUCGGmU*mG*mC*mU | |
| 83 | mN*mN*mN*(N)17GUUUUAGAmGmCmUmA(L1)mUmAmGmCAAGUUAAAAUAAGGCUA | 308 |
| | GUCCGUUAUCAC(L1)GGGCACCGAGUmCmGmGmU*mG*mC*mU | |
| 100 | mN*mN*mN*(N)17GUUUUAGAmGmCmUmAmGmAmAmAmUmAmGmCAAGUUAAAAUAAG | 309 |
| | GCUAGUCCGUUAUCAmAmCmUmUmGmAmAmAmAmAmGmUmGmGmCmAmCmCmGmAmGmU | |
| | mCmGmGmUmGmCmU*mU*mU*mU | |
| 91 | mN*mN*mN*(N)17GUUUUAGAmGmCmUmAmGmAmAmAmUmAmGmCAAGUUAAAAUAAG | 310 |
| | GCUAGUCCGUUAUCACGAAAGGGCACCGAGUCGGmU*mG*mC*mU | |
| 90 | mN*mN*mN*(N)17GUUUUAGAmGmCmUmA(L1)mUmAmGmCAAGUUAAAAUAAGGCUA | 311 |
| | GUCCGUUAUCAC(L1)GGGCACCGAGUCGGmUmGmC | |
| 90 | mN*mN*mN*(N)17GUUUUAGAmGmCmUmAmGmAmAmAmUmAmGmCAAGUUAAAAUAAG | 312 |
| | GCUAGUCCGUUAUCACGAAAGGGCACCGAGUCGGmUmGmC | |
| 84 | mN*mN*mN*(N)17GUUUUAGAmGmCmUmA(L1)mUmAmGmCAAGUUAAAAUAAGGC | 313 |
| | (L3)GUCCGUUAUCAC(L1)GGGCACCGAGUCGGmUmGmC*mU | |
| 42 | mN*mN*mN*(N)17GUUUUAGAmGmCmUmAmUmGmCmUmGmUmUmUmG*mG | 314 |
| 42 | mN*mN*mN*(N)17GUUUUAGAmGmCmUmAmUmGmCmUmGmUmUmU*mG*mG | 315 |
| 39 | mN*mN*mN*(N)17GUUUUAGAmGmCmUmAmUmGmCmUmG*mU*mG | 316 |
| 36 | mN*mN*mN*(N)17GUUUUAGAmGmCmUmAmUmG*mC*mG | 317 |
| 33 | mN*mN*mN*(N)17GUUUUAGAmGmCmU*mA*mG | 318 |
| 90 | mN*mN*mN*mNNNNNfNfNfNNfNfNNNfNfNNNmGUUUUAGmAmGmCmUmAmGmAmAm | 319 |
| | AmUmAmGmCmAAGUUmAAmAAmUmAmAGmGCUmAGUCmCmGUUAUmCAmCGmAmAmAmG | |
| | mGmCmAmCmCmGmAGUCmGmGmUmGmC | |
| 90 | mN*mN*mN*mNNNN*NfNfNfNNfNfNNNfNfNNNmGUUUUAGmAmGmCmUmAmGmAmA | 320 |
| | mAmUmAmGmCmAAGUUmAAmAAmUmAmAGmGCUmAGUCmCmGUUAUmCAmCGmAmAmAm | |
| | GmGmCmAmCmCmGmAGUCmGmGmUmGmC | |
| 74 | mN*mN*mN*NNNNNNNNNNNNNNNNNGUUUUAGAdSAAGUUAAAAUAAGGCUAGUCCGU | 321 |
| | UAUCAC(L1)GGGCACCGAGUCGGmUmGmC | |
| 74 | mN*mN*mN*NNNNNNNNNNNNNNNNNGUUUUAGA(L4)AAGUUAAAAUAAGGCUAGUCC | 322 |
| | GUUAUCAC(L1)GGGCACCGAGUCGGmUmGmC | |
| 91 | mN*mN*mN*(N)(17)mGUUUUAGmAmGmCmUmAmGmAmAmAmUmAmGmCmAAGUUmAA | 323 |
| | mAAmUmAmAGmGCUmAGUCmCmGUUAUmCAmCGmAmAmAmGGmGmCmAmCmCmGmAGUC | |
| | mGmGmU*mG*mC*mU | |
| 91 | mN*mN*mN*mNNNN*NfNfNfNNfNfNNNNNNNmGUUUUAGmAmGmCmUmAmGmAmAmAmUm | 324 |
| | AmGmCmAAGUUmAAmAAmUmAmAGmGCUmAGUCmCmGUUAUmCAmCGmAmAmAmGmGmCmAmCm | |
| | CmGmAGUCmGmGmU*mG*mC*mU | |
| 91 | mN*mN*mN*mNNNNNfNfNfNNfNNNNfNfNNNmGUUUUAGmAmGmCmUmAmGmAmAmAmUmA | 325 |
| | mGmCmAAGUUmAAmAAmUmAmAGmGCUmAGUCmCmGUUAUmCAmCGmAmAmAmGmGmCmAmCm | |
| | CmGmAGUCmGmGmU*mG*mC*mU | |
| 90 | mN*mN*mN*(N)17GUUUUAGAmGmCmUmAmGmAmAmAmUmAmGmCAAGUUAAAAUAAG | 326 |
| | GCUAGUCCGUUAUCACGAAAGGGCACCGAGUCGGmU*mG*mC | |
| 88 | (mN*)3N17GUUUUAGAmGmCmUmAmGmAmAmAmUmAmGmCAAGUUAAAAUAAGGCUAG | 327 |
| | UCCGUUAUCAACUUGGCACCGAGUCGG*mU*mG*mC | |
| 88 | (mN*)3N17GUUUUAGAmGmCmUmAmGmAmAmAmUmAmGmCAAGUUAAAAUAAGGCUAG | 328 |
| | UCCGUUAUCAAAAUGGCACCGAGUCGG*mU*mG*mC | |
| 88 | (mN*)3N17GUUUUAGAmGmCmUmAmGmAmAmAmUmAmGmCAAGUUAAAAUAAGGCUAG | 329 |
| | UCCGUUAUCAmAmAmAmUmGmGmCmAmCmCmGmAmGmUmCmGmG*mU*mG*mC | |
| 90 | (mN*)3N17GUUUUAGAmGmCmUmAmGmAmAmAmUmAmGmCAAGUUAAAAUAAGGCUAG | 330 |
| | UCCGUUAUCACGAAAGGGCACCGAGUCGG*mU*mG*mC | |
| 90 | (mN*)3N17GUUUUAGAmGmCmUmAmGmAmAmAmUmAmGmCAAGUUAAAAUAAGGCUAG | 331 |
| | UCCGUUAUCAmCmGmAmAmAmGmGmGmCmAmCmCmGmAmGmUmCmGmG*mU*mG*mC | |
| 91 | (mN*)3N17GUUUUAGAmGmCmUmAmGmAmAmAmUmAmGmCAAGUUAAAAUAAGGCUAG | 332 |
| | UCCGUUAUCACGAAAGGGCACCGAGUCGGU*mG*mC*mU | |
| 91 | (mN*)3N17GUUUUAGAmGmCmUmAmGmAmAmAmUmAmGmCAAGUUAAAAUAAGGCUAG | 333 |
| | UCCGUUAUCACGAAAGGGCACCGAGUCGGmUmGmC*mU | |
| 91 | (mN*)3N17GUUUfUAGmAmGmCmUmAmGmAmAmAmUmAmGmCmAmAGUfUmAfAmAfA | 334 |
| | mUAmAmGmGmCmUmAGUmCmCGUfUAmUmCAmCmGmAmAmAmGmGmGmCmAmCmCmGmA | |
| | mGmUmCmGmGmU*mG*mC*mU | |
| 108 | GUUUUAGAmGmC(L1)mGmCAAGUUAAAAUAAGGCUAGUCCGUUAUCAC(L1)GGGCAC | 335 |
| | CGAGUCGGmUmGmCdTdGdCdCdAdTdCdTdGdGdAdGdCdAdGdTdGdAdTCUGGmCm | |
| | CmUmGmCmA*mG | |
| 108 | mN*mN*mN*(N)17GUUUUAGAmGmC(L1)mGmCAAGUUAAAAUAAGGCUAGUCCGUUA | 336 |
| | UCAC(L1)GGGCACCGAGUCGGmUmGmCdTdGdCdCdAdTdCdTdGdGdAdGdCdAdGd | |
| | TdGdAdTCUGGmCmCmUmGmC*mA*mG | |
| 108 | mN*mN*mN*(N)17GUUUUAGAmGmC(L1)mGmCAAGUUAAAAUAAGGCUAGUCCGUUA | 337 |
| | UCAC(L1)GGGCACCGAGUCGGmUmGmCdTdGdCdCdAdTdCdTdGdGdAdGdCdAdGd | |
| | TdGdAdTCUGGmCmCmUmG*mC*mA*mG | |
| 108 | mN*mN*mN*(N)17GUUUUAGAmGmC(L1)mGmCAAGUUAAAAUAAGGCUAGUCCGUUA | 338 |
| | UCAC(L1)GGGCACCGAGUCGGmUmGmCdTdGdCdCdAdTdCdTdGdGdAdGdCdAdGd | |
| | TdGdAdTCUGmGmCmCmUmG*mC*mA*mG | |
| 108 | mN*mN*mN*(N)17GUUUUAGAmGmC(L1)mGmCAAGUUAAAAUAAGGCUAGUCCGUUA | 339 |
| | UCAC(L1)GGGCACCGAGUCGGmUmGmCdTdGdCdCdAdTdCdTdGdGdAdGdCdAdGd | |
| | TdGdAdTCUmGmGmCmCmUmG*mC*mA*mG | |
| 108 | mN*mN*mN*(N)17GUUUUAGAmGmC(L1)mGmCAAGUUAAAAUAAGGCUAGUCCGUUA | 340 |
| | UCAC(L1)GGGCACCGAGUCGGmUmGmCdTdGdCdCdAdTdCdTdGdGdAdGdCdAdGd | |
| | TdGdAdTCmUmGmGmCmCmUmG*mC*mA*mG | |
| 108 | mN*mN*mN*(N)17GUUUUAGAmGmC(L1)mGmCAAGUUAAAAUAAGGCUAGUCCGUUA | 341 |
| | UCAC(L1)GGGCACCGAGUCGGmUmGmCdTdGdCdCdAdTdCdTdGdGdAdGdCdAdGd | |
| | TdGdAdTmCmUmGmGmCmCmUmG*mC*mA*mG | |
| 109 | mN*mN*mN*(N)17GUUUUAGAmGmC(L1)mGmCAAGUUAAAAUAAGGCUAGUCCGUUA | 342 |
| | UCAC(L1)GGGCACCGAGUCGGmUmGmCdTdGdCdCdAdTdCdTdGdGdAdGdCdAdGd | |
| | TdGdAdTCUGGmCmCmUmGmCmAmG*mU | |
| 110 | mN*mN*mN*(N)17GUUUUAGAmGmC(L1)mGmCAAGUUAAAAUAAGGCUAGUCCGUUA | 343 |
| | UCAC(L1)GGGCACCGAGUCGGmUmGmCdTdGdCdCdAdTdCdTdGdGdAdGdCdAdGd | |
| | TdGdAdTCUGGmCmCmUmGmCmAmGmU*mU | |
| 111 | mN*mN*mN*(N)17GUUUUAGAmGmC(L1)mGmCAAGUUAAAAUAAGGCUAGUCCGUUA | 344 |
| | UCAC(L1)GGGCACCGAGUCGGmUmGmCdTdGdCdCdAdTdCdTdGdGdAdGdCdAdGd | |
| | TdGdAdTCUGGmCmCmUmGmCmAmGmUmU*mU | |
| 105 | mN*mN*mN*(N)17mGUUUUAGmAmGmC(L1)mGmCmAAGUUmAAmAAmUmAmAGmGCU | 345 |
| | mAGUCmCmGUUAUmCAmC(L1)mGGmGmCmAmCmCmGmAGUCmGmGmUmGmCdAdAdAd | |
| | TdAdGdAdCdAdCdGdTdAdAdTdCUUACmUmGmGmAmAmG*mU | |
| 105 | mN*mN*mN*(N)17GUUUUAGAmGmCmUmA(L1)mUmAmGmCAAGUUAAAAUAAGGCUA | 346 |
| | GUCCGUUAUCAC(L1)GGGCACCGAGUCGGmUmGmCdGdCdAdAdGdAdTdGdGdCdAd | |
| | GdCdGdCGAACmAmGmC*mU | |
| 78 | mN*mN*mN*(N)17mGUUUUAGmAmGmC(L1)mGmCmAAGUUmAAmAAmUmAmAGmGCU | 347 |
| | mAGUCmCmGUUAUmCAmC(L1)mGGmGmCmAmCmCmGmAGUCmGmGmUmGmC | |
| 82 | mN*mN*mN*(N)17GUUUUAGAmGmCmUmA(L1)mUmAmGmCAAGUUAAAAUAAGGCUA | 348 |
| | GUCCGUUAUCAC(L1)GGGCACCGAGUCGGmUmGmC | |
| 78 | mN*mN*mN*(N)17GUUUUAGAmGmC(L1)mGmCAAGUUAAAAUAAGGCUAGUCCGUUA | 349 |
| | UCAC(L1)GGGCACCGAGUCGGmUmGmC | |
| 74 | mN*mN*mN*NNNNNNNNNNNNNNNNNGUUUUAGA(L3)AAGUUAAAAUAAGGCUAGUCC | 372 |
| | GUUAUCAC(L1)GGGCACCGAGUCGGmUmGmC | |
| 110 | mN*mN*mN*(N)17GUUUUAGAmGmCmUmA(L1)mUmAmGmCAAGUUAAAAUAAGGCUA | 375 |
| | GUCCGUUAUCAC(L1)GGGCACCGAGUCGGmUmGmCdTdGdCdCdAdTdCdTdGdGdAd | |
| | GdCdAdGdTdGdAdTCUGGmCmCmUmG*mC | |
| 112 | mN*mN*mN*(N)17GUUUUAGAmGmCmUmA(L1)mUmAmGmCAAGUUAAAAUAAGGCUA | 376 |
| | GUCCGUUAUCAC(L1)GGGCACCGAGUCGGmUmGmCdTdGdCdCdAdTdCdTdGdGdAd | |
| | GdCdAdGUGAUCUGGmCmCmUmGmCmA*mG | |
| 112 | mN*mN*mN*(N)17GUUUUAGAmGmCmUmA(L1)mUmAmGmCAAGUUAAAAUAAGGCUA | 377 |
| | GUCCGUUAUCAC(L1)GGGCACCGAGUCGGmUmGmCdTdGdCdCdAdTdCdTdGdGdAd | |
| | GdCdAdGdTGAUCUGGmCmCmUmGmCmA*mG | |
| 112 | mN*mN*mN*(N)17GUUUUAGAmGmCmUmA(L1)mUmAmGmCAAGUUAAAAUAAGGCUA | 378 |
| | GUCCGUUAUCAC(L1)GGGCACCGAGUCGGmUmGmCdTdGdCdCdAdTdCdTdGdGdAd | |
| | GdCdAdGdTdGAUCUGGmCmCmUmGmCmA*mG | |
| 112 | mN*mN*mN*(N)17GUUUUAGAmGmCmUmA(L1)mUmAmGmCAAGUUAAAAUAAGGCUA | 379 |
| | GUCCGUUAUCAC(L1)GGGCACCGAGUCGGmUmGmCdTdGdCdCdAdTdCdTdGdGdAd | |
| | GdCdAdGdTdGdAUCUGGmCmCmUmGmCmA*mG | |
| 112 | mN*mN*mN*(N)17GUUUUAGAmGmCmUmA(L1)mUmAmGmCAAGUUAAAAUAAGGCUA | 380 |
| | GUCCGUUAUCAC(L1)GGGCACCGAGUCGGmUmGmCdTdGdCdCdAdTdCdTdGdGdAd | |
| | GdCdAdGdTdGdAdTdCUGGmCmCmUmGmCmA*mG | |
| 112 | mN*mN*mN*(N)17GUUUUAGAmGmCmUmA(L1)mUmAmGmCAAGUUAAAAUAAGGCUA | 381 |
| | GUCCGUUAUCAC(L1)GGGCACCGAGUCGGmUmGmCdTdGdCdCdAdTdCdTdGdGdAd | |
| | GdCdAdGdTdGdAdTdCdTGGmCmCmUmGmCmA*mG | |
| 112 | mN*mN*mN*(N)17GUUUUAGAmGmCmUmA(L1)mUmAmGmCAAGUUAAAAUAAGGCUA | 382 |
| | GUCCGUUAUCAC(L1)GGGCACCGAGUCGGmUmGmCdTdGdCdCdAdTdCdTdGdGdAd | |
| | GdCdAdGdTdGdAdTdCdTdGdGmCmCmUmGmCmA*mG | |
| 112 | mN*mN*mN*(N)17GUUUUAGAmGmCmUmA(L1)mUmAmGmCAAGUUAAAAUAAGGCUA | 383 |
| | GUCCGUUAUCAC(L1)GGGCACCGAGUCGGmUmGmCdTdGdCdCdAdTdCdTdGdGdAd | |
| | GdCdAdGdTdGdAdTdCdTdGdGdCdCmUmGmCmA*mG | |
| 77 | mN*mN*mN*NNNNNNNNNNNNNNNNNGUUUUAGA(L2)AAGUUAAAAUAAGGCUAGUCC | 431 |
| | GUUAUCAC(L1)GGGCACCGAGUCGGmUmGmC | |
| 77 | mN*mN*mN*NNNNNNNNNNNNNNNNNGUUUUAGA(L1)AAGUUAAAAUAAGGCUAGUCC | 432 |
| | GUUAUCAC(L1)GGGCACCGAGUCGGmUmGmC | |
| 82 | mN*mN*mN*NNNNNNNNNNNNNNNNNGUUUUAGAmGmCmUmA(L1)mUmAmGmCAAGUU | 433 |
| | AAAAUAAGGCUAGUCCGUUAUCAC(L1)GGGCACCGAGUmCmGmGmUmGmC | |
| | mN*mN*mN*NNNNNNNNNNNNNNNNNmGUUUUAGmAmGmCmUmAmGmAmAmAmUmAmGm | 434 |
| | CmAAGUUmAAmAAmUmAmAGmGCUmAGUCmCmGUUAUmCAmCGmAmAmAmGGmGmCmAm | |
| | CmCmGmAGUCmGmGmUmGmC | |

In Tables 1A-1D, SEQ ID NOs: 29-34, 36-37, 121-128, 251-268, 270-271, 300, 335-346, 375-383, and 446-454 comprise a guide RNA scaffold sequence and a 3′ extension sequence as described herein. SEQ ID NOs: 1-28, 35, 38-39, 40, 101-120, 129-130, 131, 201-218, 225-250, 269, 272-273, 274-276, 301-318, 323-325, 326-334, 347-349, 372-374, 431-433, 434-446 comprise a guide RNA scaffold sequence but do not comprise a 3′ extension. In some embodiments, the guide RNA comprises a guide RNA scaffold sequence comprising any one of SEQ ID NOs: 1-28, 35, 38-39, 40, 101-120, 129-130, 131, 201-218, 225-250, 269, 272-273, 274-276, 301-318, 323-325, 326-334, 347-349, 372-374, 431-433, 434-446. In some embodiments, the guide RNA comprises a nucleotide sequence comprising any one of SEQ ID NOs: 29-34, 36-37, 121-128, 251-268, 270-271, 300, 335-346, 375-383, and 446-454, but lacking the 3′ extension of that sequence. For example, in some embodiments, the guide RNA may comprise a guide RNA scaffold sequence of any one of SEQ ID NOs: 29-34, 36-37, 121-128, 251-268, 270-271, 300, 335-346, 375-383, and 446-454, but with a different 3′ extension sequence than that provided in the above sequences. As will be apparent from the disclosure provided herein, the sequence of a 3′ extension can vary depending on the desired edit to be introduced into a genomic sequence. A 3′ extension can be designed for the desired edit, according to the principles set forth herein, and the 3′ extension can, in some embodiments, be linked to a guide RNA scaffold sequence, e.g., the guide RNA scaffold sequences provided in Tables 1A-1D, e.g., SEQ ID NOs: 1-28, 35, 38-39, 40, 101-120, 129-130, 131, 201-218, 225-250, 269, 272-273, 274-276, 301-318, 323-325, 326-334, 347-349, 372-374, 431-433, 434-446.

In some embodiments, where any of the sequences in Tables 1A-ID comprise a 3′ terminal uridine, the guide RNA comprising the sequence does not comprise a 3′ terminal uridine. In some embodiments, where any of the sequences in Tables 1C-1D comprise one or more phosphorothioate (PS) bonds between the last four nucleotides of the 3′ end, the guide RNA comprising the sequence does not comprise the PS bonds between the last four nucleotides when the 3′ end of the guide RNA is covalently linked to the 3′ extension.

As used herein throughout the present disclosure for modified sequences, (mN*) 3 represents three consecutive nucleotides each having any base, a 2′-OMe, and a 3′ PS linkage to the next nucleotide; and N₁₇ or (N) 17 represent 17 consecutive N (any nucleotide), respectively. As used herein throughout the present disclosure for modified sequences, including Tables 1C and 1D or elsewhere, “m” indicates a 2′-O-Me modification, “f” indicates a 2′-fluoro modification, a “*” indicates a phosphorothioate linkage between nucleotides, “T” indicates a thymine nucleotide, “(L1)” an S18 internal linker disclosed herein (see Table 7), “(L2)” an S9 internal linker disclosed herein (see Table 7), “(L3)” denotes an S6 internal linker disclosed herein (see Table 7), “(L4)” denotes an S3 internal linker disclosed herein (see Table 7), a “(dS)” indicates an abasic site having 1′,2′-dideoxyribose modification (e.g., dSpacer from IDT). As used herein, “S3” indicates a 5′-O—CH2-CH2-CH2-O—PO3-3′ internal linker, “S6” indicates a 5′-O—(CH2O)2-PO3-3′ internal linker, “S9” indicates a 5′-O—(CH2O)3-PO3-3′ internal linker, “S18” indicates a 5′-O—(CH2O)6-PO3-3′ internal linker (the structure of such internal linkers are shown in Table 7), and no modification in the context of a modified sequence indicates an RNA (2′-OH) and phosphodiesterase linkage to the 3′ nucleotide when one is present.

In certain embodiments, the guide sequence is a chemically modified sequence. In certain embodiments, the chemically modified guide sequence is (mN*) 3 (N) 13-17. In certain embodiments, the guide sequence is (mN*)3(N)17, i.e., mN*mN*mN*NNNNNNNNNNNNNNNNN. In certain embodiments, each N of the (N)13-17 or the (N)17 is unmodified, i.e., an RNA (2′-OH and phosphodiesterase linkage to the 3′ nucleotide when one is present). In certain embodiments, each N in the (N)13-17 or the (N)17 is independently modified, e.g., independently modified with a 2′-O-methyl modification.

In some embodiments, the sgRNA disclosed herein may be modified as shown herein or in the sequence mN*mN*mN*NNNNNNNNNNNNNNNNN GUUUUAGAmGmCmUmAmGmAmAmAmUmAmGmCAAGUUAAAAUAAGGCUAGUCCGUUAU CACGAAAGGGCACCGAGUCGG*mU*mG*mC (SEQ ID NO: 193 or 330).

In some embodiments, the sgRNA disclosed herein may be modified as shown herein or in the sequence

(SEQ ID NO: 194 or 310)
mN*mN*mN*NNNNNNNNNNNNNNNNNGUUUUAGAmGmCmUmAmGmAmAm
AmUmAmGmCAAGUUAAAAUAAGGCUAGUCCGUUAUCACGAAAGGGCACC
GAGUCGGmU*mG*mC*mU.

In the case of a sgRNA, the guide sequences may further comprise a SpyCas9 sgRNA scaffold sequence. An example of a SpyCas9 sgRNA scaffold sequence is shown in the Table 5A below (SEQ ID NO: 22: GUUUUAGAGCUAGAAAUAGCAAGUUAAAAU AAGGCUAGUCCGUUAUCAACUUGAAAAAGUGGCACCGAGUCGGUGC-“Exemplary SpyCas9 sgRNA-1”), included at the 3′ end of the guide sequence, and provided with the domains as shown in the table below. LS is lower stem. B is bulge. US is upper stem. H1 and H2 are hairpin 1 and hairpin 2, respectively. Collectively H1 and H2 are referred to as the hairpin region. A model of the structure containing both a guide sequence and a scaffold sequence is provided in FIG. 10A of WO2019237069, which is incorporated herein by reference. As will be apparent from the disclosure provided herein, the present disclosure provides various alternatives to the Exemplary SpyCas9 sgRNA-1, modifications, and equivalents, as will be appreciated by those of skill in the art.

The nucleotide sequence of Exemplary SpyCas9 sgRNA-1 may serve as a template sequence for specific chemical modifications, sequence substitutions and truncations.

In certain embodiments, the guide RNA is an sgRNA or a dgRNA, for example, and it optionally comprises a chemical modification. In some embodiments, the modified sgRNA comprises a guide sequence and a SpyCas9 sgRNA sequence, e.g., Exemplary SpyCas9 sgRNA-1. A guide RNA, such as an sgRNA, may include modifications on the 5′ end of the guide sequence or on the 3′ end of the SpyCas9 sgRNA sequence, such as, e.g., Exemplary SpyCas9 sgRNA-1 at one or more of the terminal nucleotides, e.g., at 1, 2, 3, or 4 of the nucleotides at the 3′ end or at the 5′ end. In certain embodiments, the modified nucleotide is selected from a 2′-O-methyl (2′-Ome) modified nucleotide, a 2′-O-(2-methoxyethyl) (2′-O-moe) modified nucleotide, a 2′-fluoro (2′-F) modified nucleotide, a phosphorothioate (PS) linkage between nucleotides, or an inverted abasic modified nucleotide; or a combination thereof. In certain embodiments, the modified nucleotide includes a 2′-Ome modified nucleotide. In certain embodiments, the modified nucleotide includes a PS linkage. In certain embodiments, the modified nucleotide includes a 2′-Ome modified nucleotide and a PS linkage.

In certain embodiments, using SEQ ID NO: 22: GUUUUAGAGCUAGAAAUAGCAAGUUAAAAUAAGGCUAGUCCGUUAUCAACUUGAAAAAG UGGCACCGAGUCGGUGC “Exemplary SpyCas9 sgRNA-1,” see WO2019237069, the contents of which are incorporated herein by reference). The portions of the Exemplary SpyCas9 sgRNA-1 and position numbering scheme are set forth in Table 5A below.

As an example, the Exemplary SpyCas9 sgRNA-1 further includes one or more of:
A. a shortened hairpin 1 region, or a substituted and optionally shortened hairpin 1 region, wherein
   1, at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks
      a. any one or two of H1-5 through H1-8,
      b. one, two, or three of the following pairs of nucleotides: H1-1 and H1-12, H1-2 and H1-11, H1-3 and H1-10, and H1-4 and H1-9, or
      c. 1-8 nucleotides of hairpin 1 region; or
   2. the shortened hairpin 1 region lacks 4-8 nucleotides, preferably 4-6 nucleotides; and
      a. one or more of positions H1-1, H1-2, or H1-3 is deleted or substituted relative to Exemplary SpyCas9 sgRNA-1 (SEQ ID NO: 22) or
      b. one or more of positions H1-6 through H1-10 is substituted relative to Exemplary SpyCas9 sgRNA-1 (SEQ ID NO: 22); or
   3. the shortened hairpin 1 region lacks 5-10 nucleotides, preferably 5-6 nucleotides, and one or more of positions N18, H1-12, or n is substituted relative to Exemplary SpyCas9 sgRNA-1 (SEQ ID NO: 22); or
B. a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1 (SEQ ID NO: 22); or
C. a substitution relative to Exemplary SpyCas9 sgRNA-1 (SEQ ID NO: 22) at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or
D. an Exemplary SpyCas9 sgRNA-1 (SEQ ID NO: 22) with an upper stem region, wherein the upper stem modification comprises a modification to any one or more of US1-US12 in the upper stem region, wherein
   1. the modified nucleotide is optionally selected from a 2′-O-methyl (2′-Ome) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof; or
   2. the modified nucleotide optionally includes a 2′-Ome modified nucleotide.

Other SpyCas9 spacer and scaffold sequences and chemical modification patterns are provided, for example in WO2018107028, WO2019237069, and WO 2021119275, each of which is incorporated herein by reference.

In some embodiments, the scaffold of the guide RNA comprises the nucleotides of SEQ ID NO: 27 or 40. In some embodiments, (a) nucleotides 5′-GUUUUAGAGCUAGAAAUAGCAAGUUAAAAU-3′ or 5′-GUUUUAGACGUAGAAAUACGAAGUUAAAAU-3′ constitute a repeat/antirepeat (R/AR) region, wherein nucleotides 1-6 (LS1-LS6) and 25-30 (LS7-LS12) of the R/AR constitute a lower stem (LS) region, nucleotides 7-8 (B1-B2) and 21-24 (B3-B6) of the R/AR constitute a bulge region; and nucleotides 9-20 (US1-US12) of the R/AR constitute an upper stem (US) region; (b) nucleotides 5′-AAGGCUAGUCCGUUAUCA-3′ constitute a nexus region (N1-N18); (c) nucleotides 5′-CGAAAG-3′ constitute a hairpin 1 (H1) region (from 5′ to 3′, H1-2, H1-5 to H1-8, and H1-11); (d) nucleotides 5′-GCACCGAGUCGGUGC-3′ constitute a hairpin 2 (H2) region H2-1 to H2-15); and (e) a G nucleotide between H1 region and H2 region constitutes nucleotide n; wherein the scaffold comprises at least one region with unmodified and modified nucleotides selected from: (i) the lower stem region comprising modified nucleotides at LS1, LS8, LS10, and LS12; nucleotides not modified with 2′-O-Me at LS2, LS3, LS4, LS5; (ii) the bulge region comprising nucleotides not modified with 2′-O-Me at B5 and B6; (iii) the upper stem region comprising modified nucleotides at at least 10 positions selected from US1, US2, US3, US4, US5, US6, US7, US8, US9, US10, US11, and US12, optionally at all positions US1, US2, US3, US4, US5, US6, US7, US8, US9, US10, US11, and US12; (iv) the nexus region comprising modified nucleotides at N1, N2, N4, N7, N11, N12, and N17; and nucleotides not modified with 2′-O-Me at N6, N8, N9, N10, N13, and N14; (v) the hairpin 1 (H1) region, comprising modified nucleotides at H1-2, H1-6, H1-7, H1-8, and H1-11; and nucleotides not modified with 2′-O-Me at H1-5; (vi) the hairpin 2 (H2) region comprising modified nucleotides at H2-1, H2-2, H2-3, H2-4, H2-5, H2-6, H2-7, H2-12, H2-13, H2-14, and H2-15; and nucleotides not modified with 2′-O-Me at H2-8, H2-9, and H2-10; and (vii) the n is a modified or unmodified nucleotide.

In some embodiments, the scaffold comprises at least one region with unmodified and modified nucleotides selected from: (a) the lower stem region comprising modified nucleotides at LS1, LS8, LS10, and LS12; nucleotides not modified with 2′-O-Me at LS2, LS3, LS4, LS5, and LS6; (b) the bulge region comprising nucleotides not modified with 2′-O-Me at B5 and B6; (c) the upper stem region comprising modified nucleotides at at least 10 positions selected from US1, US2, US3, US4, US5, US6, US7, US8, US9, US10, US11, and US12, optionally at all positions US1, US2, US3, US4, US5, US6, US7, US8, US9, US10, US11, and US12; (d) the nexus region comprising modified nucleotides at N1, N2, N4, N7, N11, N12, and N17; and nucleotides not modified with 2′-O-Me at N6, N8, N9, N10, N13, N14, and N16; (e) the hairpin 1 (H1) region, comprising modified nucleotides at H1-2, H1-6, H1-7, H1-8, and H1-11; and nucleotides not modified with 2′-O-Me at H1-5; (f) the hairpin 2 (H2) region comprising modified nucleotides at H2-1, H2-2, H2-3, H2-4, H2-5, H2-6, H2-7, H2-11, H2-12, H2-13, H2-14, and H2-15; and nucleotides not modified with 2′-O-Me at H2-8, H2-9, and H2-10; and (g) the n is a modified or unmodified nucleotide.

In some embodiments, the scaffold comprises at least one region with unmodified and modified nucleotides selected from: (a) the lower stem region comprising modified nucleotides at LS1, LS8, LS10, and LS12; nucleotides not modified with 2′-O-Me at LS2, LS3, LS4, LS5, LS6, and LS9; (b) the bulge region comprising nucleotides not modified with 2′-O-Me at B5 and B6; (c) the upper stem region comprising modified nucleotides at at least 10 positions selected from US1, US2, US3, US4, US5, US6, US7, US8, US9, US10, US11, and US12, optionally at all positions US1, US2, US3, US4, US5, US6, US7, US8, US9, US10, US11, and US12; (d) the nexus region comprising modified nucleotides at N1, N2, N4, N7, N11, N12, N16, and N17; and nucleotides not modified with 2′-O-Me at N6, N8, N9, N10, N13, N14, and N16; (e) the hairpin 1 (H1) region, comprising modified nucleotides at H1-2, H1-6, H1-7, H1-8, and H1-11; and nucleotides not modified with 2′-O-Me at H1-5; (f) the hairpin 2 (H2) region comprising modified nucleotides at H2-1, H2-2, H2-3, H2-4, H2-5, H2-6, H2-7, H2-11, H2-12, H2-13, H2-14, and H2-15; and nucleotides not modified with 2′-O-Me at H2-8, H2-9, and H2-10; and (g) the n is a modified or unmodified nucleotide. In some embodiments, the lower stem region comprises modified nucleotides at LS1, LS8, LS10, and LS12; nucleotides not modified with 2′-O-Me at LS2, LS3, LS4, LS5, LS6, LS7, LS9, and LS11.

In some embodiments, the lower stem region comprises unmodified nucleotides at LS2, LS3, LS4, and LS5, optionally at LS2, LS3, LS4, LS5, LS6, LS7, LS9, and LS11. In some embodiments, the lower stem region comprises unmodified nucleotides at LS7, LS9, and LS11. In certain embodiments, the sgRNA comprises a modified nucleotide at one or more positions L1, L8, L10, and L12, and comprises an unmodified nucleotide at one or more positions LS2, LS3, LS4, LS5, LS6, LS7, LS9, and LS11. In certain embodiments, the sgRNA comprises a modified nucleotide at one, two, three, or four of positions

L1, L8, L10, and L12, and comprises an unmodified nucleotide at one, two, three, four, five, six, seven, or eight positions LS2, LS3, LS4, LS5, LS6, LS7, LS9, and LS11. In some embodiments, the sgRNA comprises a modified nucleotide at one or more positions L1, L8, L10, and L12 and comprises an unmodified nucleotide at each of positions LS2, LS3, LS4, LS5, LS6, LS7, LS9, and LS11. In some embodiments, L1, L8, L10, and L12, when modified, comprise a 2′-O-Me modification. In certain embodiments, L1, L8, L10, and L12, when modified, independently comprises a 2′-O-Me modification or a 2′F modified nucleotide. In certain embodiments, the sgRNA comprises a 2′-O-Me at L1, L8, L10, and L12, and an unmodified nucleotide at LS2, LS3, LS4, LS5, LS6, LS7, LS9, and LS11.

In some embodiments, the bulge region comprises modified nucleotides at B2 and B3. In some embodiments, the bulge region comprises unmodified nucleotides at B5 and B6, optionally at B1, B4, B5, and B6. In certain embodiments, one or more of B1, B4, B5, and B6 comprise unmodified nucleotides. In some embodiments, the bulge region comprises a modified nucleotide at one or both of positions B2 and B3 and comprises an unmodified nucleotide at each of positions B1, B4, B5, and B6. In some embodiments, B2 and B3, when modified, independently comprise a 2′-O-Me modification or a 2′-F modified nucleotide. In certain embodiments, the buldge region comprises a 2′-O-Me modified nucleotide at each of positions B2 and B3 each comprise, and comprise unmodified nucleotides at each of positions B1, B4, B5, and B6.

In some embodiments, the modified nucleotides of the upper stem are 2′-O-Me modified nucleotides. In some embodiments, the nexus region comprises unmodified nucleotides at N6, N8, N9, N10, N13, N14, and N18, optionally at N3, N5, N6, N8, N9, N10, N13, N14, N15, and N18. In some embodiments, the nexus region comprises unmodified nucleotides at N3, N5, N6, N8, N9, N10, N13, N14, N15, and N18. In certain embodiments, N1, N2, N4, N7, N11, N12, and N17 comprise modified nucleotides. In certain embodiments, N1, N2, N4, N7, N11, N12, and N17 comprise 2′-O-Me modified nucleotides. In certain embodiments, each of positions N3, N5, N6, N8, N9, N10, N13, N14, N15, N16, and N18 comprise unmodified nucleotides. In some embodiments, the nexus region comprises a 2′-O-Me modified nucleotide at each of positions N1, N2, N4, N7, N11, N12, and N17 and comprises an unmodified nucleotide at each of positions N3, N5, N6, N8, N9, N10, N13, N14, N15, N16, and N18. In certain embodiments, positions N1, N2, N4, N7, N11, N12, and N17 comprise modified nucleotides, and positions N3, N5, N6, N8, N9, N10, N13, N14, N15, N16, and N18 are not modified with 2′-O-Me.

In some embodiments, the hairpin 1 region comprises an unmodified nucleotide at H1-5. In certain embodiments, all of H1-2, H1-6, H1-7, H1-8, and H1-11 comprise modified nucleotides, optionally 2′-O-Me modified nucleotides. In certain embodiments, positions H1-2, H1-6, H1-7, H1-8, and H1-11 comprise 2′-O-Me modified nucletodies and position H1-5 comprises a nucleotide that is not modified. In certain embodiments, positions H2-1, H2-2, H2-3, H2-4, H2-5, H2-6, H2-7, H2-11, H2-12, H2-13, H2-14, and H2-15 comprise 2′-O-Me modified nucleotides and positions H2-8, H2-9, and H2-10 comprise a nucleotide that is not modified. In certain embodiments, positions H1-2, H1-6, H1-7, H1-8, and H1-11 comprise 2′-O-Me modified nucletodies and position H1-5 comprises a nucleotide that is not modified; and positions H2-1, H2-2, H2-3, H2-4, H2-5, H2-6, H2-7, H2-11, H2-12, H2-13, H2-14, and H2-15 comprise 2′-O-Me modified nucleotides and positions H2-8, H2-9, and H2-10 comprise a nucleotide that is not modified.

In some embodiments, the hairpin 2 region comprises unmodified nucleotides at H2-8, H2-9, and H2-10. In some embodiments, the gRNA further comprises a 3′ tail. In some embodiments, the gRNA comprises a 3′ end modification.

In some embodiments, the scaffold sequence comprises an internal linker. In some embodiments, wherein the scaffold sequence comprises (i) an internal linker in or substitutes for the upper stem region; (ii) an internal linker in a nexus region; or (iii) an internal linker in the hairpin 1 region. In some embodiments, the guide RNA comprises (a) the sequence of mGUUUUAGmAmGmC(L1)mGmCmAAGUUmAAmAAmUmAmAGmGCUmAGUCmCmGUUAUm CAmC(L1)mGGmGmCmAmCmCmGmAGUCmGmGmUmGmC (SEQ ID NO: 272) or a sequence having at least 90%, 95%, or 98% identity to SEQ ID NO: 272; (b) the sequence of GUUUUAGAmGmCmUmA(L1)mUmAmGmCAAGUUAAAAUAAGGCUAGUCCGUUAUCAC(L1)G GGCACCGAGUCGGmUmGmC (SEQ ID NO: 211) or a sequence having at least 90%, 95%, or 98% identity to SEQ ID NO: 211; (c) the sequence of GUUUUAGAmGmC(L1)mGmCAAGUUAAAAUAAGGCUAGUCCGUUAUCAC(L1)GGGCACCGA GUCGGmUmGmC (SEQ ID NO: 232) or a sequence having at least 90%, 95%, or 98% identity to SEQ ID NO: 232; wherein L1 denotes that the linker is S18, and L3 denotes that the linker is S6; or (d) the sequence of mGUUUUAGmAmGmCmUmA(L1)mUmAmGmCmAAGUUmAAmAAmUmAmAGmGCUmAGUCm CmGUUAUmCAmC(L1)mGGmGmCmAmCmCmGmAGUCmGmGmUmGmC (SEQ ID NO: 224) or a sequence having at least 90%, 95%, or 98% identity to SEQ ID NO: 224; wherein L1 denotes that the linker is S18. In some embodiments, the spacer or the scaffold of the guide RNA comprises a modified nucleotide.

gRNAs; Domains/Regions Thereof

In some embodiments, a guide RNA provided herein is an sgRNA. Briner A E et al., Molecular Cell 56:333-339 (2014) describes functional domains of sgRNAs, referred to herein as “domains”, including the “spacer” domain responsible for targeting, the “lower stem”, the “bulge”, “upper stem” (which may include a tetraloop), the “nexus”, and the “hairpin 1” and “hairpin 2” domains. See Briner et al. at page 334, FIG. 1A. As described in detail elsewhere herein, one or more domains (e.g., hairpin 1 or the upper stem) may be shortened in an sgRNA described herein.

In some embodiments, the sgRNA comprises a guide region and a conserved portion 3′ to the guide region (also referred to as a “scaffold region” or “scaffold” elsewhere herein), wherein the conserved portion comprises a repeat-anti-repeat region, a nexus region, a hairpin 1 region, and a hairpin 2 region. The repeat-anti-repeat region comprises an upper stem region and a lower stem region. Table 5A provides a schematic of the domains of an sgRNA as used herein. In Table 5A, the “n” between regions represents a variable number of nucleotides, for example, from 0 to 1, 2, 3, 4, 5, 6, 7, 8, 9, 10, 11, 12, 13, 14, 15, 16, 17, 18, 19, 20, or more. In some embodiments, n equals 0. In some embodiments, n equals 1.

In some embodiments, the sgRNA comprises at least one of: a first internal linker substituting for at least 4 nucleotides of the upper stem region; a second internal linker substituting for 2 nucleotides of the nexus region; and a third internal linker substituting for at least 2 nucleotides of the hairpin 1.

In some embodiments, the sgRNA comprises the first internal linker and the second internal linker. In some embodiments, the sgRNA comprises the first internal linker and the third internal linker. In some embodiments, the sgRNA comprises the second internal linker and the second internal linker. In some embodiments, the sgRNA comprise the first internal linker, the second internal linker, and the second internal linker.

In some embodiments, the first internal linker has a bridging length of about 9-30 atoms, optionally about 15-21 atoms. In some embodiments, the first internal linker substitutes for 4, 5, 6, 7, 8, 9, 10, 11, or 12 nucleotides of the repeat-anti-repeat region of the guide RNA.

In some embodiments, the second internal linker has a bridging length of about 9-15 atoms. In some embodiments, the second internal linker substitutes for a hairpin region of the nexus region of the sgRNA. In some embodiments, the second internal linker substitutes for 2 nucleotides of a stem region of the nexus region of the sgRNA.

In some embodiments, the third internal linker has a bridging length of about 9-30 atoms, optionally about 15-21 atoms. In some embodiments, the third internal linker substitutes for 4, 5, 6, 7, 8, 9. 10, 11, or 12 nucleotides of the hairpin 1 of the guide RNA.

In some embodiments, the first internal linker is in a hairpin between a first portion and a second portion, and the first portion and the second portion together form a duplex portion.

In some embodiments, the third internal linker is in a hairpin between a first portion of the guide RNA and second portion of the guide RNA, and the first portion and the second portion together form a duplex portion.

In some embodiments, a hairpin 2 region of the guide RNA does not contain any internal linker.

### 5′ Terminus Region

The sgRNA can comprise nucleotides at the 5′ end to the scaffold sequence as shown in Table 5A. The 5′ terminus of the sgRNA comprises a spacer or guide region that functions to direct a Cas protein, e.g., a SpyCas9 protein, to a target nucleotide sequence. The length of the spacer region can vary from 17-20 nucleotides in length, with the nucleotide sequence being dependent on the target sequence within the genome. In some embodiments, the spacer region comprises 17, 18, 19, or 20 nucleotides. For the purpose of defining nucleotide positions therein, the spacer region nucleotides are numbered using the designation 5′-S1 to S20-3′ (as shown in FIGS. 2A-2B).

### Lower Stem

The sgRNA comprises a lower stem (LS) region that when viewed linearly, is separated by a bulge and upper stem regions. See Table 5A.

In some embodiments, the lower stem regions comprise 1-12 nucleotides, e.g. in one embodiment the lower stem regions comprise LS1-LS12. In some embodiments, the lower stem region comprises fewer nucleotides than shown in Table 5A. In some embodiments, the lower stem region comprises more nucleotides than shown in Table 5A. When the lower stem region comprises fewer or more nucleotides than shown in the schematic of Table 5A, the modification pattern, as will be apparent to the skilled artisan, should be maintained.

In some embodiments, the lower stem region has nucleotides that are complementary in nucleic acid sequence when read in opposite directions. In some embodiments, the complementarity in nucleic acid sequence of lower stem leads to a secondary structure of a stem in the sgRNA (e.g., the regions may base pair with one another). In some embodiments, the lower stem regions may not be perfectly complimentary to each other when read in opposite directions.

### Bulge

The sgRNA comprises a bulge region typically comprising six nucleotides, B1-B6. When viewed linearly, when an upper stem is present, the bulge region is separated into two regions. See Table 5A. In some embodiments, the bulge region comprises six nucleotides, wherein the first two nucleotides are followed by an upper stem region, followed by the last four nucleotides of the bulge. In some embodiments, the bulge region comprises fewer nucleotides than shown in Table 5A. In some embodiments, the bulge region comprises more nucleotides than shown in Table 5A. When the bulge region comprises fewer or more nucleotides than shown in the schematic of Table 5A, the modification pattern, as will be apparent to the skilled artisan, should be maintained. In some embodiments, the bulge region includes an internal spacer and no upper stem region as discussed below.

In some embodiments, the presence of a bulge results in a directional kink between the upper and lower stem modules in an sgRNA.

### Upper Stem

In some embodiments, the upper stem region is a shortened upper stem region, such as any of the shortened upper stem regions described elsewhere herein. In some embodiments, no upper stem is present.

In other embodiments, the sgRNA comprises an upper stem region comprising 12 nucleotides. In some embodiments, the upper stem region comprises a loop sequence. In some instances, the loop is a tetraloop (loop consisting of four nucleotides). In some embodiments, the upper stem region comprises more nucleotides than shown in Table 5A.

When the upper stem region comprises fewer or more nucleotides than shown in the schematic of Table 5A, the modification pattern, as will be apparent to the skilled artisan, should be maintained.

The upper stem region has nucleotides that are complementary in nucleic acid sequence when read in opposite directions. In some embodiments, the complementarity in nucleic acid sequence of upper stem leads to a secondary structure of a stem in the sgRNA (e.g., the regions may base pair with one another). In some embodiments, the upper stem regions may not be perfectly complimentary to each other when read in opposite directions.

In some embodiments, the upper stem region comprises fewer nucleotides than shown in FIG. 10A of WO2019237069, and sometimes is not present. In certain embodiments, bulge nucleotides B2 and B3 (corresponding to nucleotides 8 and 21 of SEQ ID: 400; see Table 5A) are directly joined (i.e., such that no intervening nucleotides are present) by an internal linker. In certain embodiments, B2 and B3 are directly joined by one or more, e.g., 1, 2, 3, or 4 abasic nucleosides. In certain embodiments, B2 and B3 are joined by an internal linker or one or more, e.g., 1, 2, 3, or 4, abasic nucleosides wherein additional nucleotides present do not form a duplex portion above the bulge. In certain embodiments, B2 and B3 are joined by an internal linker or one or more, e.g., 1, 2, 3, or 4 abasic nucleoside wherein additional nucleotides present do not form a duplex portion longer than 3 nucleotides above the bulge.

### Nexus

The sgRNA comprises a nexus region that is located between the lower stem region and the hairpin 1 region. In some embodiments, the nexus comprises 18 nucleotides. In some embodiments, the nexus region comprises nucleotides N1 through N18 as shown in Table 5A. In some embodiments, the nexus region comprises a substitution (e.g., at position N18) or lacks a nucleotide, such as any of the nexus regions with a substitution or lacking a nucleotide described in detail elsewhere herein.

In some embodiments, the nexus region comprises fewer nucleotides than shown in Table 5A. In some embodiments, the nexus region comprises more nucleotides than shown in Table 5A. When the nexus region comprises fewer or more nucleotides than shown in the schematic of Table 5A, the modification pattern, as will be apparent to the skilled artisan, should be maintained.

The nexus region has nucleotides that are complementary in nucleic acid sequence when read in opposite directions. In some embodiments, the complementarity in nucleic acid sequence leads to a secondary structure of a stem or stem loop in the sgRNA (e.g., certain nucleotides in the nexus region may base pair with one another). In some embodiments, the nexus regions may not be perfectly complimentary to each other when read in opposite directions.

### Hairpins

In some embodiments, the sgRNA comprises a hairpin structure within the hairpin region. The hairpin region is downstream of (i.e., 3′ to) the repeat-anti-repeat region and the nexus region. The region of nucleotides immediately downstream of the nexus region is termed “hairpin 1” or “H1”. The region of nucleotides 3′ to hairpin 1 is termed “hairpin 2” or “H2”. The hairpin region comprises both hairpin 1 and hairpin 2. In some embodiments, the sgRNA comprises hairpin 1 or hairpin 2.

In some embodiments, the hairpin 1 region is a shortened hairpin 1 region, such as any of the shortened hairpin 1 regions described elsewhere herein. In some embodiments, a shortened hairpin 1 region includes an internal linker as discussed below.

In other embodiments, the hairpin 1 region comprises 12 nucleotides immediately downstream of the nexus region. In some embodiments, the hairpin 1 region comprises nucleotides H1-1 through H1-12 as shown in Table 5A.

In some embodiments, the hairpin 2 region comprises 15 nucleotides downstream of the hairpin 1 region. In some embodiments, the hairpin 2 region comprises nucleotides H2-1 through H2-15 as shown in Table 5A.

In some embodiments, one or more nucleotides is present between the hairpin 1 and the hairpin 2 regions. The one or more nucleotides between the hairpin 1 and hairpin 2 region may be modified or unmodified. In some embodiments, hairpin 1 and hairpin 2 are separated by one nucleotide. In some embodiments, the hairpin regions comprise fewer nucleotides than shown in Table 5A. In some embodiments, the hairpin regions comprise more nucleotides than shown in Table 5A. When a hairpin region comprises fewer or more nucleotides than shown in the schematic of Table 5A, the modification pattern, as will be apparent to the skilled artisan, should be maintained.

A hairpin region has nucleotides that are complementary in nucleic acid sequence when read in opposite directions. In some embodiments, the hairpin regions may not be perfectly complimentary to each other when read in opposite directions (e.g., the top or loop of the hairpin comprises unpaired nucleotides).

### 3′ Terminus

The sgRNA has a 3′ end, which is the last nucleotide of the scaffold region of the SpyCas9 guide sgRNA. The 3′ terminus region includes the last 1-4 nucleotides from the 3′ end. In some embodiments, the 3′ end is the end of hairpin 2. In some embodiments, the sgRNA comprises nucleotides after the hairpin region(s). In some embodiments, the sgRNA includes a 3′ tail region, in which case the last nucleotide of the 3′ tail is the 3′ terminus. In some embodiments, the 3′ tail region comprises 1, 2, 3, or 4 nucleotides that are not associated with the secondary structure of a hairpin. In some embodiments, the 3′ tail region comprises 1 nucleotide that is not associated with the secondary structure of a hairpin. In some embodiments, the 3′ tail region comprises 1, 2, or 3 nucleotides that are not associated with the secondary structure of a hairpin. It is understood that a 3′ extension is distinct, structurally and functionally, from a 3′ tail.

gRNAs Comprising Linkers

In certain embodiments, the SpyCas9 guide RNA scaffold sequence an internal linker. In some embodiments, the SpyCas9 guide RNA scaffold sequence comprises an internal linker in the upper stem region; an internal linker in a nexus region; or an internal linker in the hairpin 1 region. In certain embodiments, the SpyCas9 guide RNA scaffold sequence comprises an internal linker in the upper stem region and an internal linker in the hairpin 1 region. The length of an internal linker may be dependent on, for example, the number of nucleotides replaced by the linker and the position of the linker in the guide RNA. Internal linkers and their use in the context of guide RNA are provided in WO2022261292, the content of which is incorporated by reference.

gRNAs disclosed herein may comprise an internal linker. In general, any internal linker compatible with the function of the guide RNA may be used. It may be desirable for the linker to have a degree of flexibility. In some embodiments, the internal linker comprises at least two, three, four, five, six, or more on-pathway single bonds. A bond is on-pathway if it is part of the shortest path of bonds between the two nucleotides whose 5′ and 3′ positions are connected to the linker.

As used herein the length of the internal linker can be defined by its bridging length. The “bridging length” of an internal linker as used herein refers to the distance or number of atoms in the shortest chain of atoms on the pathway from the first atom of the linker (bound to a 3′ substituent, such as an oxygen or phosphate, of the preceding nucleotide to the last atom of the linker (bound to a 5′ substituent, such as an oxygen or phosphate) of the following nucleotide) (e.g., from ~ to # in the structure of Formula (I) described below). Approximate predicted bridging lengths for various linkers are provided in a table below.

Exemplary predicted linker lengths by number of atoms, number of ethylene glycol units, approximate linker length in Angstroms on the assumption that an ethylene glycol monomer is about 3.7 Angstroms, and suitable location for substitution of at least the entire loop portion of a hairpin structure are provided in Table 2 below. Substitution of two nucleotides requires a linker length of at least about 11 Angstroms. Substitution of at least 3 nucleotides requires a linker length of at least about 16 Angstroms.

In some embodiments, the internal linker has a bridging length of about 3 Angstroms-37 Angstroms. In some embodiments, the internal linker has a bridging length of about 3-23 Angstroms.

TABLE 2

| Number of | Number of Ethylene Glycol | Approximate length in |
|---|---|---|
| atoms | units | Angstroms |
| 3 | 1 | 3.7 |
| 6 | 2 | 7.4 |
| 9 | 3 | 11.1 |
| 12 | 4 | 14.8 |
| 15 | 5 | 18.5 |
| 18 | 6 | 22.2 |
| 21 | 7 | 25.9 |
| 24 | 8 | 29.6 |
| 27 | 9 | 33.3 |
| 30 | 10 | 37 |

In some embodiments, the internal linker comprises a structure of formula (I):

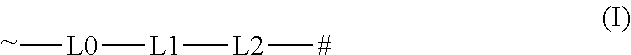

wherein:
~ indicates a bond to a 3′ substituent of the preceding nucleotide; # indicates a bond to a 5′ substituent of the following nucleotide;
L0 is null or C1-3 aliphatic;
L1 is -[E¹-(R¹)]ₘ—, where
each R¹ is independently a C1-5 aliphatic group, optionally substituted with 1 or 2 E²,
each E¹ and E² are independently a hydrogen bond acceptor, or are each independently chosen
from cyclic hydrocarbons, and heterocyclic hydrocarbons, and
each m is 1, 2, 3, 4, 5, 6, 7, 8, 9, or 10; and
L2 is null, C₁₋₃ aliphatic, or is a hydrogen bond acceptor.

In some embodiments, L1 comprises a —CH₂CH₂O—, CH₂OCH₂—, or —OCH₂CH₂— unit (“ethylene glycol subunits”). In some embodiments, the number of —CH₂CH₂O—, —CH₂OCH₂—, or —OCH₂CH₂— units is in the range of 1, 2, 3, 4, 5, 6, 7, 8, 9 or 10.

In some embodiments, m is 1, 2, 3, 4 or 5. In some embodiments, m is 1, 2, or 3. In some embodiments, m is 6, 7, 8, 9, or 10.

In some embodiments, L0 is null. In some embodiments, L0 is —CH₂— or —CH₂CH₂—.

In some embodiments, L2 is null. In some embodiments, L2 is —O—, —S—, or C₁₋₃ aliphatic. In some embodiments, L2 is —O—. In some embodiments, L2 is —S—. In some embodiments, L2 is —CH₂— or —CH₂CH₂—.

In the tables herein, L1 and L2, are optionally, C9 and C18, respectively as follows:

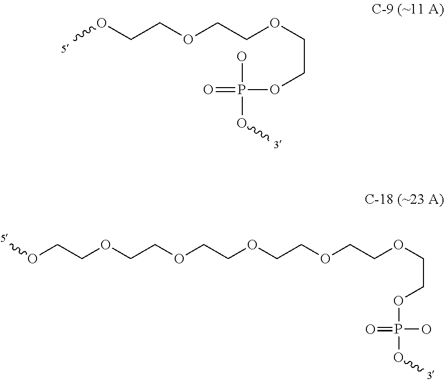

In certain embodiments, the internal linker has a bridging length of about 3-30 atoms, optionally 3-21 atoms, and the linker substitutes for at least 2 nucleotides of the guide RNA. In certain embodiments, the internal linker has a bridging length of about 3-18 atoms, and the linker substitutes for at least 2 nucleotides of the guide RNA. In certain embodiments, the internal linker substitutes for 2-12 nucleotides.

In some embodiments, the guide RNA comprises a nucleic acid sequence of SEQ ID NO: 22, including modifications disclosed elsewhere herein. Table 3A shows various embodiments of the guide RNA structures and species with possible number of internal linkers and positions.

TABLE 3A

| gRNA | | # internal | |
|---|---|---|---|
| structures | Type | linkers | Positions of internal linkers |
| Repeat/anti- | Spy | 3 | Repeat/Anti-Repeat region; nexus |
| R; nexus; | | | (within hairpin or replace hairpin), |
| Hp1; Hp2 | | | Hairpin 1 (Hp1) |
| Repeat/anti- | Spy | 2 | Any two of Repeat/Anti-R; nexus |
| R; nexus; | | | (within hairpin or replace hairpin), Hp1 |
| Hp1; Hp2 | | | |
| Repeat/anti- | Spy | 1 | Any of Repeat/Anti-R; nexus (within |
| R; nexus; | | | hairpin or replace hairpin), Hp1 |
| Hp1; Hp2 | | | |

In certain embodiments, one of the internal linkers is in the repeat/anti-repeat region of the guide RNA. In certain embodiments, the internal linker substitutes for at least 4 nucleotides of the repeat-anti-repeat region of the guide RNA. In certain embodiments, the internal linker substitutes for the loop in the repeat-anti-repeat region of a SpyCas9 guide RNA, corresponding to nucleotides 13-16 in SEQ ID NO: 22.

In some embodiments, wherein the internal linker is in a repeat-anti-repeat region of the guide RNA, the internal linker is in the upper stem region. In certain embodiments, the internal linker in the upper stem region substitutes for US1 through US12 relative to SEQ ID NO: 22.

In certain embodiments, the SpyCas9 guide RNA scaffold sequence further comprises an internal linker in a nexus region. In certain embodiments, the internal linker substitutes for 2, 3, or 4 nucleotides of the nexus region of the guide RNA. In certain embodiments, the internal linker substitutes for the loop in the nexus region of a SpyCas9 guide RNA corresponding to nucleotides 33-36 of SEQ ID NO: 22.

In certain embodiments, the internal linker is in a hairpin region of the guide RNA. In certain embodiments, the internal linker substitutes for at least 4 nucleotides of the hairpin region of the guide RNA. In certain embodiments, the internal linker or one of the internal linkers is in the hairpin 1 region. In certain embodiments, the internal linker substitutes for H1-5 through H1-8 relative to SEQ ID NO: 22.

In certain embodiments, nucleotides 29-40 (US1 through US12 of the upper stem region) are substituted for the first internal linker relative to SEQ ID NO: 22; and nucleotides 70-78 (HP1-HP10 of the hairpin 1 region) are substituted for the second internal linker relative to SEQ ID NO: 22.

In some embodiments, the guide RNA comprises the sequence of GUUUUAGAGCUA-linker-UAGCAAGUUAAAAUAAGGCUAGUCCGUUAUCAC-linker-GGGCACCGAGUCGGUGC (SEQ ID NO: 299), wherein “linker” denotes an internal linker.

In some embodiments, the guide RNA comprises the sequence of GUUUUAGA(L3) AAGUUAAAAUAAGGCUAGUCCGUUAUCAC (L1) GGGCACCGAGUCGGUGC U (SEQ ID NO: 370) or a sequence having at least 90%, 95%, or 98% identity to SEQ ID NO: 370, wherein “(L1)” denotes an S18 internal linker disclosed herein (see Table 7), “(L3)” denotes an S6 internal linker disclosed herein (see Table 7).

In some embodiments, the guide RNA comprises the sequence of GUUUUAGA(L3) AAGUUAAAAUAAGGCUAGUCCGUUAUCAC (L1) GGGCACCGAGUCGGUGC U (SEQ ID NO: 370), or GUUUUAGA(L3) AAGUUAAAAUAAGGCUAGUCCGUUAUCAC (L1) GGGCACCGAGUCGGUGC (SEQ ID NO: 373), wherein “(L1)” denotes an S18 internal linker disclosed herein (see Table 7), “(L3)” denotes an S6 internal linker disclosed herein (see Table 7).

In some embodiments, the guide RNA comprises mN*mN*mN*NNNNNNNNNNNNNNNNNGUUUUAGA(L3)AAGUUAAAAUAAGGCUAGUCCGU UAUCAC(L1)GGGCACCGAGUCGGmU*mG*mC*mU (SEQ ID NO: 371) or a sequence having at least 90%, 95%, 98%, or 99% identity to SEQ ID NO: 371, wherein * denotes that the nucleotide is linked to the next nucleotide with a PS bond, a lower case “m” denotes that the nucleotide is modified with 2′-O-Me, “(L1)” denotes an S18 internal linker disclosed herein (see Table 7), “(L3)” denotes an S6 internal linker disclosed herein (see Table 7), where N is any nucleotide.

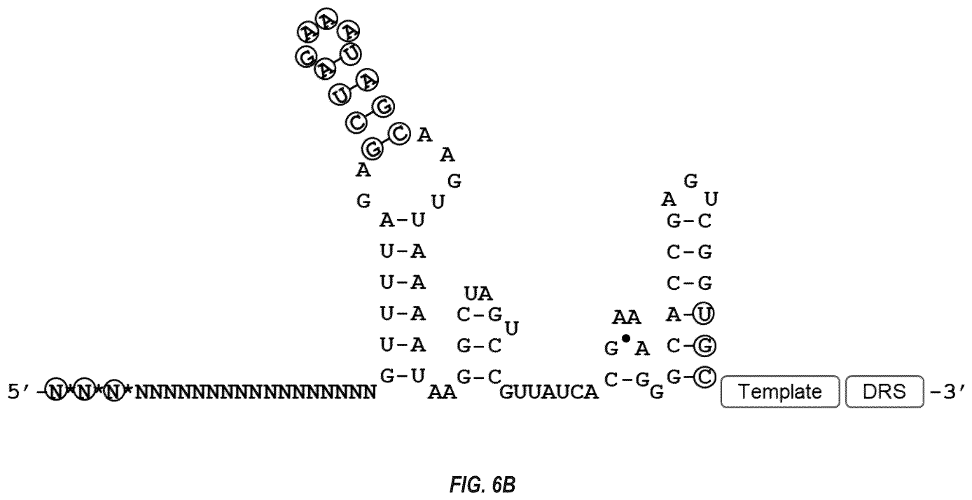

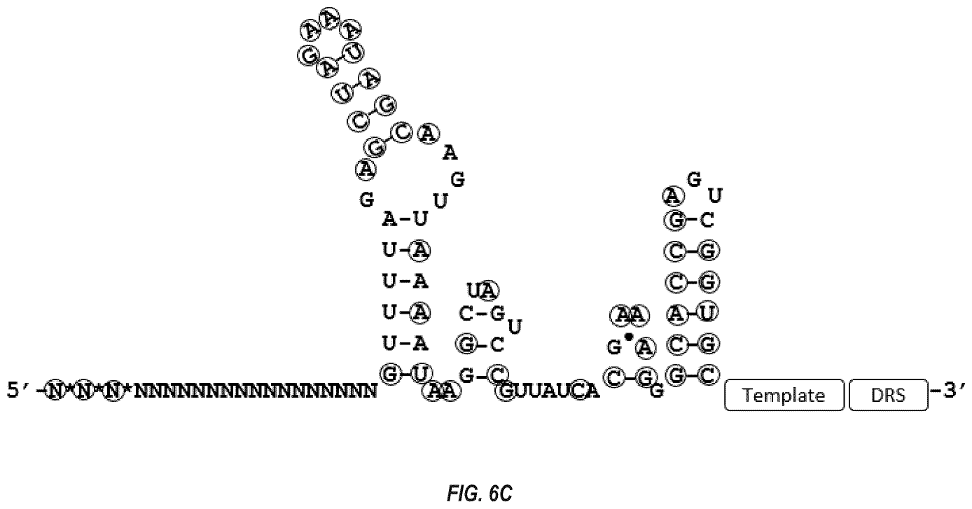

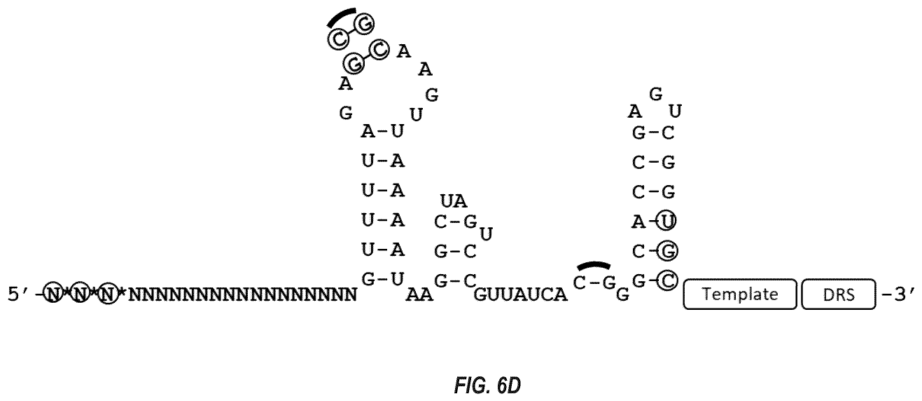

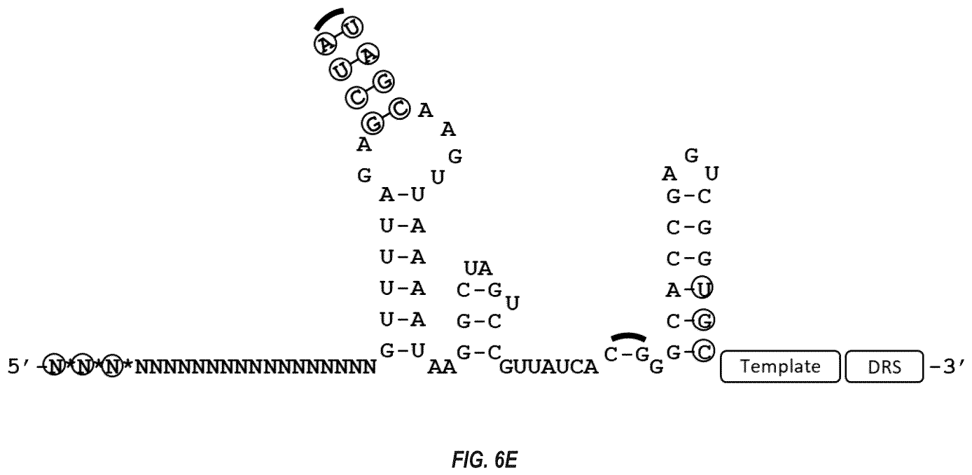

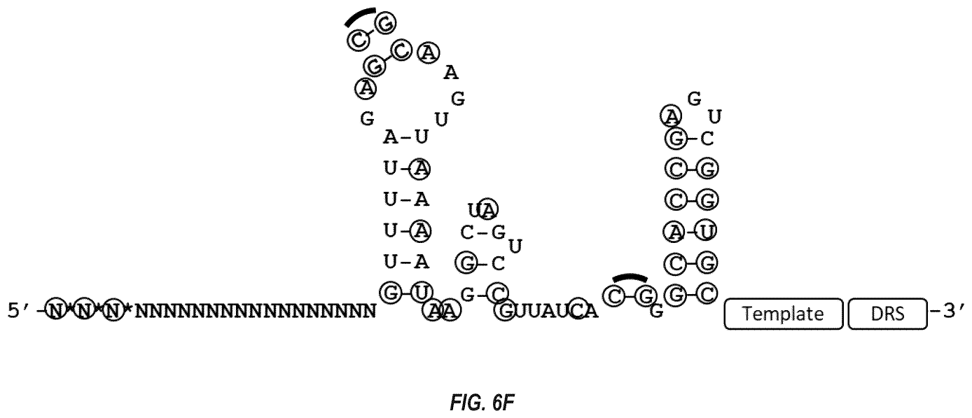

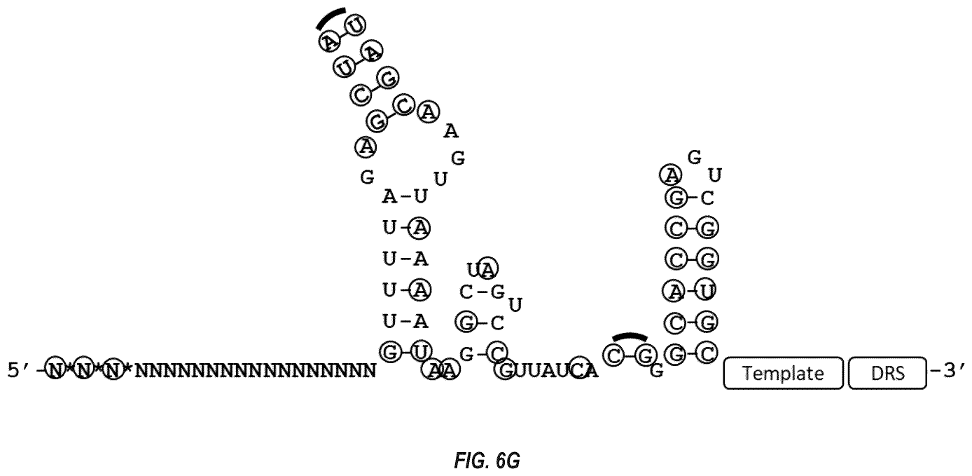

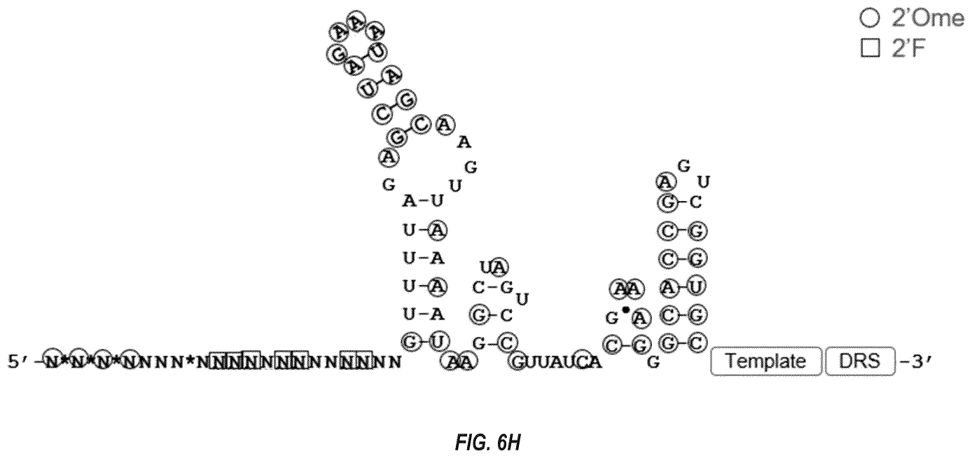

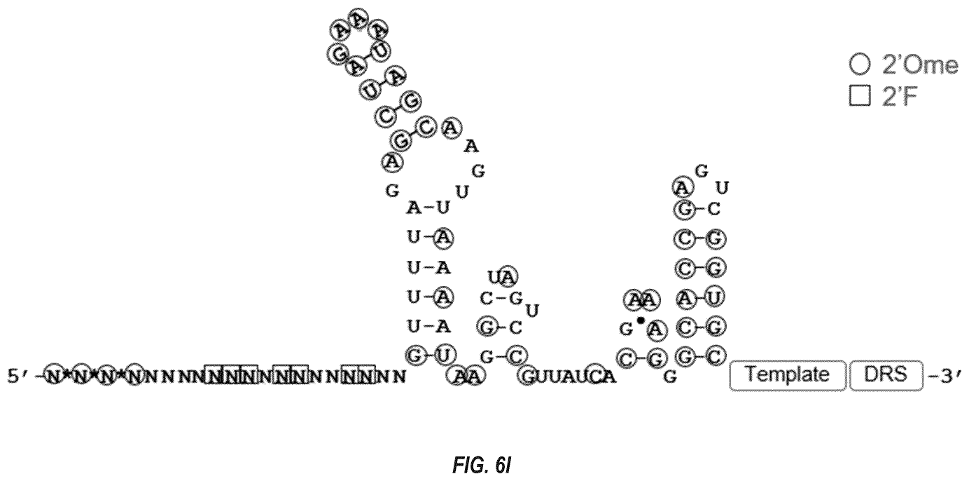

In some embodiments, the guide RNA comprises mN*mN*mN*NNNNNNNNNNNNNNNNNGUUUUAGA(L3) AAGUUAAAAUAAGGCUAGUCCGU UAUCAC (L1) GGGCACCGAGUCGGmU*mG*mC*mU (SEQ ID NO: 371), wherein * denotes that the nucleotide is linked to the next nucleotide with a PS bond, a lower case “m” denotes that the nucleotide is modified with 2′-O-Me, “(L1)” denotes an S18 internal linker disclosed herein (see Table 7), “(L3)” denotes an S6 internal linker disclosed herein (see Table 7), where N is any nucleotide. An example of the above guide RNA is shown in FIG. 6.

In some embodiments, the guide RNA comprises GUUUUAGA(L3) AAGUUAAAAUAAGGCUAGUCCGUUAUCAC (L1) GGGCACCGAGUCGGmU* mG*mC*mU (SEQ ID NO: 203); or mN*mN*mN*NNNNNNNNNNNNNNNNNGUUUUAGA(L3) AAGUUAAAAUAAGGCUAGUCCGU UAUCAC (L1) GGGCACCGAGUCGGmU*mG*mC (SEQ ID NO: 384), wherein * denotes that the nucleotide is linked to the next nucleotide with a PS bond, a lower case “m” denotes that the nucleotide is modified with 2′-O-Me, “(L1)” denotes an S18 internal linker disclosed herein (see Table 7), “(L3)” denotes an S6 internal linker disclosed herein (see Table 7), where N is any nucleotide.

In some embodiments, the guide RNA comprises a scaffold comprising GUUUUAGA(L3) AAGUUAAAAUAAGGCUAGUCCGUUAUCAC (L1) GGGCACCGAGUCGGmU* mG*mC*mU (SEQ ID NO: 203) or mGmUmUmUmUmAmGmA(L3)mAmAmGmUmUmAmAmAmAmUmAmAmGmGmCmUmAmGmU mCmCmGmUmUmAmUmCAC(L1)GmGGCACCGAGUCGGmU*mG*mC*mU (SEQ ID NO: 372), wherein * denotes that the nucleotide is linked to the next nucleotide with a PS bond, a lower case “m” denotes that the nucleotide is modified with 2′-O-Me, “(L1)” denotes an S18 internal linker disclosed herein (see Table 7), “(L3)” denotes an S6 internal linker disclosed herein (see Table 7). In some embodiments, the SpyCas9 guide RNA comprises a modified nucleotide sequence having at least 90%, 95%, 98% identity to any one of SEQ ID NOs: 350-371 or any one of the sequences listed in Table 3B.

TABLE 3B
Exemplary SpyCas9 guide RNAs comprising linkers

| SEQ ID | |
|---|---|
| NO: | gRNA sequence |
| 350 | NNNNNNNNNNNNNNNNNNNNGUUUUAGAGCUA(L1)UAGCAAGUUAAAAUAAGGC(L2)GUCC |
| | GUUAUCAACUU(L1)AAGUGGCACCGAGUCGGUGCUUUU |
| 351 | NNNNNNNNNNNNNNNNNNNNGUUUUAGAGCUA(L1)UAGCAAGUUAAAAUAAGGC(L2)GUCC |
| | GUUAUCAC(L1)GGGCACCGAGUCGGUGC |
| 352 | mN*mN*mN*NNNNNNNNNNNNNNNNNGUUUUAGAmGmCmUmA(L1)mUmAmGmCAAGUUAAAA |
| | UAAGGC(L2)GUCCGUUAUCAC(L1)GGGCACCGAGUCGG*mU*mG*mC |
| 353 | mN*mN*mN*NNNNNNNNNNNNNNNNNGUUUUAGAmGmCmUmA(L1)mUmAmGmCAAGUUAAAA |
| | UAAGGC(L2)GUCCGUUAUCA(L1)GGCACCGAGUCGG*mU*mG*mC |
| 354 | mN*mN*mN*NNNNNNNNNNNNNNNNNGUUUUAGAmGmCmU(L1)mAmGmCAAGUUAAAAUAAG |
| | GC(L2)GUCCGUUAUCAC(L1)GGGCACCGAGUCGG*mU*mG*mC |
| 355 | mN*mN*mN*NNNNNNNNNNNNNNNNNGUUUUAGAmGmCmU(L1)mAmGmCAAGUUAAAAUAAG |
| | GC(L2)GUCCGUUAUCA(L1)GGCACCGAGUCGG*mU*mG*mC |
| 356 | mN*mN*mN*NNNNNNNNNNNNNNNNNGUUUUAGAmGmCmU(L1)mAmGmCAAGUUAAAAUAAG |
| | GC(L2)GUCCGUUAUCA(L1)GGCACCGAGUCGG*mU*mG*mC |
| 357 | mN*mN*mN*NNNNNNNNNNNNNNNNNGUUUUAGAmGmCmU(L1)mAmGmCAAGUUAAAAUAAG |
| | GC(L2)GUCCGUUAUCA(L1)GGCACCGAGUCGG*mU*mG*mC |
| 358 | mN*mN*mN*NNNNNNNNNNNNNNNNNGUUUUAGAmGmCmUmA(L1)mUmAmGmCAAGUUAAAA |
| | UAAGGC(L2)GUCCGUUAUCAC(L1)GGGCACCGAGUCGG*mU*mG*mC |
| 359 | mN*mN*mN*NNNNNNNNNNNNNNNNNGUUUUAGAmGmCmUmA(L1)mUmAmGmCAAGUUAAAA |
| | UAAGGC(L2)GUCCGUUAUCA(L1)GGCACCGAGUCGG*mU*mG*mC |
| 360 | mN*mN*mN*NNNNNNNNNNNNNNNNNGUUUUAGAmGmCmU(L1)mAmGmCAAGUUAAAAUAAG |
| | GC(L2)GUCCGUUAUCAC(L1)GGGCACCGAGUCGG*mU*mG*mC |
| 361 | mN*mN*mN*NNNNNNNNNNNNNNNNNGUUUUAGAmGmCmU(L1)mAmGmCAAGUUAAAAUAAG |
| | GC(L2)GUCCGUUAUCA(L1)GGCACCGAGUCGG*mU*mG*mC |
| 362 | mN*mN*mN*NNNNNNNNNNNNNNNNNGUUUUAGA(dS)AAGUUAAAAUAAGGCUAGUCCGUUA |
| | UCAC(L1)GGGCACCGAGUCGGmU*mG*mC*mU |
| 363 | mN*mN*mN*NNNNNNNNNNNNNNNNNGUUUUAGA(L4)AAGUUAAAAUAAGGCUAGUCCGUUA |
| | UCAC(L1)GGGCACCGAGUCGGmU*mG*mC*mU |
| 364 | mN*mN*mN*NNNNNNNNNNNNNNNNNGUUUUAGA(L3)AAGUUAAAAUAAGGCUAGUCCGUUA |
| | UCAC(L1)GGGCACCGAGUCGGmU*mG*mC*mU |
| 365 | mN*mN*mN*NNNNNNNNNNNNNNNNNGUUUUAGA(L2)AAGUUAAAAUAAGGCUAGUCCGUUA |
| | UCAC(L1)GGGCACCGAGUCGGmU*mG*mC*mU |
| 366 | mN*mN*mN*NNNNNNNNNNNNNNNNNGUUUUAGA(L1)AAGUUAAAAUAAGGCUAGUCCGUUA |
| | UCAC(L1)GGGCACCGAGUCGGmU*mG*mC*mU |
| 367 | mN*mN*mN*NNNNNNNNNNNNNNNNNGUUUUAGAmGmC(L1)mGmCAAGUUAAAAUAAGGCUA |
| | GUCCGUUAUCAC(L1)GGGCACCGAGUCGGmU*mG*mC*mU |
| 368 | mN*mN*mN*NNNNNNNNNNNNNNNNNGUUUUAGAmGmCmUmA(L1)mUmAmGmCAAGUUAAAA |
| | UAAGGCUAGUCCGUUAUCAC(L1)GGGCACCGAGUCGGmU*mG*mC*mU |
| 369 | mN*mN*mN*NNNNNNNNNNNNNNNNNGUUUUAGAmGmCmUmA(L1)mUmAmGmCAAGUUAAAA |
| | UAAGGCUAGUCCGUUAUCAC(L1)GGGCACCGAGUmCmGmGmU*mG*mC*mU |
| 370 | GUUUUAGA(L3)AAGUUAAAAUAAGGCUAGUCCGUUAUCAC(L1)GGGCACCGAGUCGGUGCU |
| 371 | mN*mN*mN*NNNNNNNNNNNNNNNNNGUUUUAGA(L3)AAGUUAAAAUAAGGCUAGUCCGUUA |
| | UCAC(L1)GGGCACCGAGUCGGmU*mG*mC*mU |
| 373 | GUUUUAGA(L3)AAGUUAAAAUAAGGCUAGUCCGUUAUCAC(L1)GGGCACCGAGUCGGUGC |
| 374 | GUUUUAGA(L3)AAGUUAAAAUAAGGCUAGUCCGUUAUCAC(L1)GGGCACCGAGUCGGmU*m |
| | G*mC* |
| 384 | mN*mN*mN*NNNNNNNNNNNNNNNNNGUUUUAGA(L3)AAGUUAAAAUAAGGCUAGUCCGUUA |
| | UCAC(L1)GGGCACCGAGUCGGmU*mG*mC |
| 385 | mN*mN*mN*NNNNNNNNNNNNNNNNNGUUUUAGAdSAAGUUAAAAUAAGGCUAGUCCGUUAUC |
| | AC(L1)GGGCACCGAGUCGGmUmGmCUCUGCGAUCACGUGCmUmCmAmGmUmCmU*mG |
| 386 | mN*mN*mN*NNNNNNNNNNNNNNNNNGUUUUAGA(L4)AAGUUAAAAUAAGGCUAGUCCGUUA |
| | UCAC(L1)GGGCACCGAGUCGGmUmGmCUCUGCGAUCACGUGCmUmCmAmGmUmCmU*mG |
| 387 | mN*mN*mN*NNNNNNNNNNNNNNNNNGUUUUAGA(L3)AAGUUAAAAUAAGGCUAGUCCGUUA |
| | UCAC(L1)GGGCACCGAGUCGGmUmGmCUCUGCGAUCACGUGCmUmCmAmGmUmCmU*mG |
| 388 | mN*mN*mN*NNNNNNNNNNNNNNNNNGUUUUAGA(L2)AAGUUAAAAUAAGGCUAGUCCGUUA |
| | UCAC(L1)GGGCACCGAGUCGGmUmGmCUCUGCGAUCACGUGCmUmCmAmGmUmCmU*mG |
| 389 | mN*mN*mN*NNNNNNNNNNNNNNNNNGUUUUAGA(L1)AAGUUAAAAUAAGGCUAGUCCGUUA |
| | UCAC(L1)GGGCACCGAGUCGGmUmGmCUCUGCGAUCACGUGCmUmCmAmGmUmCmU*mG |
| 390 | mN*mN*mN*NNNNNNNNNNNNNNNNNGUUUUAGAmGmC(L1)mGmCAAGUUAAAAUAAGGCUA |
| | GUCCGUUAUCAC(L1)GGGCACCGAGUCGGmUmGmCUCUGCGAUCACGUGCmUmCmAmGmUmC |
| | mU*mG |
| 391 | mN*mN*mN*NNNNNNNNNNNNNNNNNGUUUUAGAmGmCmUmA(L1)mUmAmGmCAAGUUAAAA |
| | UAAGGCUAGUCCGUUAUCAC(L1)GGGCACCGAGUCGGmUmGmCUCUGCGAUCACGUGCmUmC |
| | mAmGmUmCmU*mG |
| 392 | mN*mN*mN*NNNNNNNNNNNNNNNNNGUUUUAGAmGmCmUmA(L1)mUmAmGmCAAGUUAAAA |
| | UAAGGCUAGUCCGUUAUCAC(L1)GGGCACCGAGUmCmGmGmUmGmCUCUGCGAUCACGUGCm |
| | UmCmAmGmUmCmU*mG |
| 393 | GUUUUAGAdSAAGUUAAAAUAAGGCUAGUCCGUUAUCAC(L1)GGGCACCGAGUCGGmUmGmC |
| | UCUGCGAUCACGUGCmUmCmAmGmUmCmU*mG |
| 394 | GUUUUAGA(L4)AAGUUAAAAUAAGGCUAGUCCGUUAUCAC(L1)GGGCACCGAGUCGGmUmG |
| | mCUCUGCGAUCACGUGCmUmCmAmGmUmCmU*mG |
| 395 | GUUUUAGA(L3)AAGUUAAAAUAAGGCUAGUCCGUUAUCAC(L1)GGGCACCGAGUCGGmUmG |
| | mCUCUGCGAUCACGUGCmUmCmAmGmUmCmU*mG |
| 396 | GUUUUAGA(L2)AAGUUAAAAUAAGGCUAGUCCGUUAUCAC(L1)GGGCACCGAGUCGGmUmG |
| | mCUCUGCGAUCACGUGCmUmCmAmGmUmCmU*mG |
| 397 | GUUUUAGA(L1)AAGUUAAAAUAAGGCUAGUCCGUUAUCAC(L1)GGGCACCGAGUCGGmUmG |
| | mCUCUGCGAUCACGUGCmUmCmAmGmUmCmU*mG |
| 398 | GUUUUAGAmGmC(L1)mGmCAAGUUAAAAUAAGGCUAGUCCGUUAUCAC(L1)GGGCACCGAG |
| | UCGGmUmGmCUCUGCGAUCACGUGCmUmCmAmGmUmCmU*mG |
| 399 | GUUUUAGAmGmCmUmA(L1)mUmAmGmCAAGUUAAAAUAAGGCUAGUCCGUUAUCAC(L1)GG |
| | GCACCGAGUCGGmUmGmCUCUGCGAUCACGUGCmUmCmAmGmUmCmU*mG |
| 400 | GUUUUAGAmGmCmUmA(L1)mUmAmGmCAAGUUAAAAUAAGGCUAGUCCGUUAUCAC(L1)GG |
| | GCACCGAGUmCmGmGmUmGmCUCUGCGAUCACGUGCmUmCmAmGmUmCmU*mG |
| 429 | GUUUUAGAGC(L1)GCAAGUUAAAAUAAGGCUAGUCCGUUAUCAC(L1)GGGCACCGAGUCGG |
| | UGC |
| 430 | GUUUUAGAGCUA(L1)UAGCAAGUUAAAAUAAGGCUAGUCCGUUAUCAC(L1)GGGCACCGAG |
| | UCGGUGC |
| 435 | NNNNNNNNNNNNNNNNNNNNGUUUUAGA(L3)AAGUUAAAAUAAGGCUAGUCCGUUAUCAC |
| | (L1)GGGCACCGAGUCGGUGCU |
| 436 | NNNNNNNNNNNNNNNNNNNNGUUUUAGAGC(L1)GCAAGUUAAAAUAAGGCUAGUCCGUUAUC |
| | AC(L1)GGGCACCGAGUCGGUGC |
| 437 | NNNNNNNNNNNNNNNNNNNNGUUUUAGAGCUA(L1)UAGCAAGUUAAAAUAAGGCUAGUCCGU |
| | UAUCAC(L1)GGGCACCGAGUCGGUGC |
| 438 | mN*mN*mN*NNNNNNNNNNNNNNNNNmGUUUUAGmAmGmCmUmA(L1)mUmAmGmCmAAGUUm |
| | AAmAAmUmAmAGmGCUmAGUCmCmGUUAUmCAmC(L1)mGGmGmCmAmCmCmGmAGUCmGmGm |
| | UmGmC |
| 439 | GUUUUAGA(ds)AAGUUAAAAUAAGGCUAGUCCGUUAUCAC(L1)GGGCACCGAGUCGGmU*mG*mC*m |
| | U |
| 440 | GUUUUAGA(L4)AAGUUAAAAUAAGGCUAGUCCGUUAUCAC(L1)GGGCACCGAGUCGGmU*mG*mC*m |
| | U |
| 441 | GUUUUAGA(L3)AAGUUAAAAUAAGGCUAGUCCGUUAUCAC(L1)GGGCACCGAGUCGGmU*mG*mC*m |
| | U |
| 442 | GUUUUAGA(L2)AAGUUAAAAUAAGGCUAGUCCGUUAUCAC(L1)GGGCACCGAGUCGGmU*mG*mC*m |
| | U |
| 443 | GUUUUAGA(L1)AAGUUAAAAUAAGGCUAGUCCGUUAUCAC(L1)GGGCACCGAGUCGGmU*mG*mC*m |
| | U |
| 444 | GUUUUAGAmGmC(L1)mGmCAAGUUAAAAUAAGGCUAGUCCGUUAUCAC(L1)GGGCACCGAGUCGGmU |
| | *mG*mC*mU |
| 445 | GUUUUAGAmGmCmUmA(L1)mUmAmGmCAAGUUAAAAUAAGGCUAGUCCGUUAUCAC(L1)GGGCACCG |
| | AGUCGGmU*mG*mC*mU |
| 446 | GUUUUAGAmGmCmUmA(L1)mUmAmGmCAAGUUAAAAUAAGGCUAGUCCGUUAUCAC(L1)GGGCACCG |
| | AGUmCmGmGmU*mG*mC*mU |
| 447 | GUUUUAGA(ds)AAGUUAAAAUAAGGCUAGUCCGUUAUCAC(L1)GGGCACCGAGUCGGmUmGmCUCUG |
| | CGAUCACGUGCmUmCmAmGmUmCmU*mG |
| 448 | GUUUUAGA(L4)AAGUUAAAAUAAGGCUAGUCCGUUAUCAC(L1)GGGCACCGAGUCGGmUmGmCUCUG |
| | CGAUCACGUGCmUmCmAmGmUmCmU*mG |
| 449 | GUUUUAGA(L3)AAGUUAAAAUAAGGCUAGUCCGUUAUCAC(L1)GGGCACCGAGUCGGmUmGmCUCUG |
| | CGAUCACGUGCmUmCmAmGmUmCmU*mG |
| 450 | GUUUUAGA(L2)AAGUUAAAAUAAGGCUAGUCCGUUAUCAC(S18)GGGCACCGAGUCGGmUmGmCUCU |
| | GCGAUCACGUGCmUmCmAmGmUmCmU*mG |
| 451 | GUUUUAGA(L1)AAGUUAAAAUAAGGCUAGUCCGUUAUCAC(S18)GGGCACCGAGUCGGmUmGmCUCU |
| | GCGAUCACGUGCmUmCmAmGmUmCmU*mG |
| 452 | GUUUUAGAmGmC(L1)mGmCAAGUUAAAAUAAGGCUAGUCCGUUAUCAC(S18)GGGCACCGAGUCGGm |
| | UmGmCUCUGCGAUCACGUGCmUmCmAmGmUmCmU*mG |
| 453 | GUUUUAGAmGmCmUmA(L1)mUmAmGmCAAGUUAAAAUAAGGCUAGUCCGUUAUCAC(L1)GGGCACCG |
| | AGUCGGmUmGmCUCUGCGAUCACGUGCmUmCmAmGmUmCmU*mG |
| 454 | GUUUUAGAmGmCmUmA(L1)mUmAmGmCAAGUUAAAAUAAGGCUAGUCCGUUAUCAC(L1)GGGCACCG |
| | AGUmCmGmGmUmGmCUCUGCGAUCACGUGCmUmCmAmGmUmCmU*mG |

In some embodiments, where any of the sequences in Table 3B comprise a 3′ terminal uridine, the guide RNA comprising the sequence does not comprise a 3′ terminal uridine.

In certain embodiments, Exemplary SpyCas9 sgRNA-1, or an sgRNA, such as an sgRNA comprising an Exemplary SpyCas9 sgRNA-1. In certain embodiments, the Exemplary SpyCas9 sgRNA-1 does not include a 3′ tail. In certain embodiments, the Exemplary SpyCas9 sgRNA-1, further may include a 3′ tail, e.g., a 3′ tail of 1, 2, 3, 4, or more nucleotides, e.g., a tail of 1 nucleotide, e.g., a tail of 1 uridine nucleotide. It is understood that a 3′ tail is not a 3′ extension disclosed herein, which includes a template and a DRS, is distinct in structure and function from a 3′ tail. The nucleotides and nucleotide modifications discussed in relation to the Exemplary SpyCas9 sgRNA-1 spacer and scaffold may or may not be tolerated in a 3′ extension as discussed further below.

### Methods of Making

Methods of producing or synthesizing a guide RNA comprising an internal linker disclosed herein are known in the art. Suitable precursors, e.g., linker can be introduced into an sgRNA oligonucleotide by using the corresponding phosphoramidite building block in methods of making sgRNA in a single production or synthetic process. Such building blocks are commercially available or can be prepared by known methods.

Methods of production or synthesis include a series of sequential coupling reactions including covalently linking a first nucleotide to a second nucleotide; covalently linking an internal linker to a second nucleotide; and covalently linking a third nucleotide to the internal linker. In certain embodiments, such linkages are performed using phosphoramidite chemistry. In certain embodiments, the method includes covalent linkage of a second linker to the first linker prior to covalent linkage of the third nucleotide.

In some embodiments, a solid support covalently attached to the linker of the guide RNA disclosed herein is provided.

The guide RNA provided herein with internal linkers are made in a single production or synthetic process such that a full-length guide RNA strand (sgRNA, crRNA, or trRNA) is produced by the production or synthetic method. In the case of a dgRNA, the crRNA and trRNA are produced or synthesized separately and annealed. That is, when the guide RNA is made as a dgRNA, the separately produced or synthesized portions do not require covalent linkage to form a stable guide RNA. In certain embodiments, the crRNA and trRNA of a dgRNA containing an internal linker as provided herein, does not include a covalent linkage between the crRNA and the trRNA.

In preferred embodiments, the guide RNA is not made using click chemistry.

### Guide RNAs Comprising Chemical Modifications

In some embodiments, the Exemplary SpyCas9 sgRNA-1 gRNA is chemically modified. An Exemplary SpyCas9 sgRNA-1, SpyCas9 spacer and scaffold, or more simply guide RNA comprising one or more modified nucleosides or nucleotides is called a “modified” guide RNA or “chemically modified” guide RNA, to describe the presence of one or more non-naturally or naturally occurring components or configurations that are used instead of or in addition to the canonical A, G, C, and U residues. In some embodiments, a modified guide RNA that is produced or synthesized with a non-canonical nucleoside or nucleotide, is here called “modified.” Modified nucleosides and nucleotides can include one or more of: (i) alteration, e.g., replacement, of one or both of the non-linking phosphate oxygens or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) modification or replacement of a naturally occurring nucleobase, including with a non-canonical nucleobase (an exemplary base modification); and (iv) modification of nucleotides at the 3′ end or 5′ end of the oligonucleotide, e.g., to provide exonuclease stability, e.g., with 2′ O-me, or 2′ halide substituted ribose; or inverted abasic terminal nucleotide, or replacement of phosphodiester with phosphorothioate. The RNA modification used depends, at least in part, to the type of the guide RNA (e.g., sgRNA, tgRNA) and the portion of the guide (e.g., spacer, scaffold, DRS, template when RNA is present in the template).

Chemical modifications such as those listed above can be combined to provide modified guide RNAs comprising nucleosides and nucleotides (collectively “residues”) that can have two, three, four, or more modifications. For example, a modified residue can have a modified sugar and a modified nucleobase. In certain embodiments, phosphate groups of a guide RNA molecule are replaced with phosphorothioate groups. In some embodiments, modified guide RNAs comprise at least one modified residue at or near the 5′ end of the RNA. In some embodiments, modified guide RNAs comprise at least one modified residue at or near the 3′ end of the RNA. In some embodiments, when the modified guide RNA comprises a 3′ extension that is covalently linked to the 3′ end of the scaffold sequence, the modified guide RNA does not comprise a modified residue at or near the 3′ end of the scaffold sequence.

In some embodiments, the guide RNA disclosed herein comprises one, two, three or more modified residues. In some embodiments, at least 5% (e.g., at least 5%, 10%, 15%, preferably at least 20%, 25%, 30%, 35%, 40%, 45%, 50%, 55%, or 60%) of the positions in a modified guide RNA are modified nucleosides or nucleotides. In some embodiments, 20-60% of the positions in the modified guide RNA spacer and scaffold are modified nucleotides.

Unmodified nucleic acids can be prone to degradation by, e.g., intracellular nucleases or nucleases found in serum. For example, nucleases can hydrolyze nucleic acid phosphodiester bonds. Accordingly, in one aspect the guide RNAs described herein can contain one or more modified nucleosides or nucleotides, e.g., to introduce stability toward intracellular or serum-based nucleases. In some embodiments, the modified guide RNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, borano phosphate esters, methyl phosphonates, phosphoroamidates, phosphodithioate, alkyl or aryl phosphonates and phosphotriesters. In certain embodiments, the modified phosphate group is a phosphorothioate group. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications, e.g., an amide linkage. In some embodiments, the charged phosphate group can be replaced by a neutral moiety. Examples of moieties which can replace the phosphate group can include, without limitation, e.g., methyl phosphonate, carboxymethyl, carbamate, amide, and thioether. Further examples of moieties which can replace the phosphate group can Include, without limitation, e.g., ethylene oxide linker, sulfonate, sulfonamide, thioformacetal, formacetal, methyleneimino, methylenemethylimino, methylenehydrazo, methylenedimethylhydrazo, and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine, and peptide nucleic acid (PNA) nucleoside surrogates.

The modified nucleosides and modified nucleotides can include one or more modifications to the sugar group, i.e., a sugar modification. For example, the 2′ hydroxyl group (OH) can be modified, e.g., replaced with a number of different “oxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH2CH₂O),CH2CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino).

Locked nucleic acids can be depicted as follows:

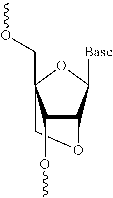

In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative). 2′ modifications can include hydrogen (i.e. deoxyribose sugars); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g., L-nucleosides. As used herein, a single abasic sugar is not understood to result in a discontinuity of a duplex.

In certain embodiments, 2′ modifications, include, for example, modifications include 2′-Ome, 2′-F, LNA, optionally 2′-O-Me.

The modified nucleosides and modified nucleotides described herein, which can be incorporated into a modified nucleic acid, can include a modified base, also called a modified nucleobase. Examples of nucleobases include, but are not limited to, adenine (A), guanine (G), cytosine (C), and uridine (U). These nucleobases can be modified or wholly replaced to provide modified residues that can be incorporated into modified nucleic acids. The nucleobase of the nucleotide can be independently selected from a purine, a pyrimidine, a purine analog, or a pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA or tracr RNA. In some embodiments, when the modified tracrRNA comprises a 3′ extension that is covalently linked to the 3′ end of the scaffold sequence, the modified tracrRNA does not comprise a modified residue at or near the 3′ end of the scaffold sequence. In embodiments comprising an sgRNA, one or more residues at one or both ends of the sgRNA may be chemically modified, or internal nucleosides may be modified, or the sgRNA may be chemically modified throughout. Certain embodiments comprise a 5′ end modification. Certain embodiments comprise a 3′ end modification. Certain embodiments comprise a 5′ end modification and a 3′ end modification. In some embodiments, when the modified sgRNA comprises a 3′ extension that is covalently linked to the 3′ end of the scaffold sequence, the modified sgRNA does not comprise a modified residue at or near the 3′ end of the scaffold sequence.

In some embodiments, the guide RNAs, spacer and scaffold, disclosed herein comprise one of the modification patterns disclosed in WO2018107028, the contents of which are hereby incorporated by reference in their entirety. In some embodiments, the guide RNAs disclosed herein comprise one of the structures/modification patterns disclosed in US20170114334, the contents of which are hereby incorporated by reference in their entirety. In some embodiments, the guide RNAs disclosed herein comprise one of the structures/modification patterns disclosed in WO2017136794, the contents of which are hereby incorporated by reference in their entirety. In some embodiments, the guide RNAs disclosed herein comprise one of the structures/modification patterns disclosed in WO2019237069, the contents of which are hereby incorporated by reference in their entirety. In some embodiments, the guide RNAs disclosed herein comprise one of the structures/modification patterns disclosed in WO2021119275, the contents of which are hereby incorporated by reference in their entirety. In some embodiments, the guide RNAs disclosed herein comprise one of the structures/modification patterns disclosed in WO2023081687, the contents of which are hereby incorporated by reference in their entirety. In some embodiments, the guide RNAs disclosed herein comprise one of the structures/modification patterns disclosed in WO2022/261292, the contents of which are hereby incorporated by reference in their entirety.

Any of the modifications described below may be present in the guide RNAs and mRNAs described herein.

In the context of chemically modified sequences, “A,” “C,” “G,” “N,” and “U” denote an RNA nucleotide, i.e., 2′-OH with a phosphodiesterase linkage to the 3′ nucleotide.

The terms “mA,” “mC,” “mU,” or “mG” are used to denote an adenine, cytosine, uridine, or guanidine nucleotide, respectively, that has been modified with 2′-O-Me.

Modification of 2′-O-methyl can be depicted as follows:

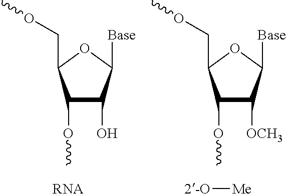

In some embodiments, the modification may be 2′-O-(2-methoxyethyl) (2′-O-moe). Modification of a ribonucleotide as a 2′-O-moe ribonucleotide can be depicted as follows:

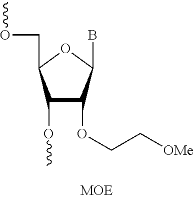

The terms “moeA,” “moeC,” “moeU,” or “moeG” may be used to denote an adenine, cytosine, uridine, or guanidine nucleotide, respectively, that has been modified with 2′-O-moe.

Another chemical modification that has been shown to influence nucleotide sugar rings is halogen substitution. For example, 2′-fluoro (2′-F) substitution on nucleotide sugar rings can increase oligonucleotide binding affinity and nuclease stability.

In this application, the terms “fA,” “fC,” “fU,” or “fG” are used to denote an adenine, cytosine, uridine, or guanidine nucleotide, respectively, that has been substituted with 2′-F.

Substitution of 2′-F can be depicted as follows:

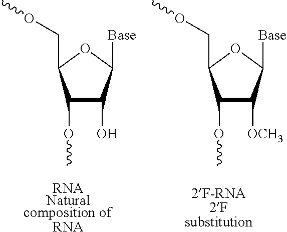

Phosphorothioate (PS) linkage or bond refers to a bond where a sulfur is substituted for one non-bridging phosphate oxygen in a phosphodiester linkage, for example in the bonds between nucleotide bases. When phosphorothioates are used to generate oligonucleotides, the modified oligonucleotides may also be referred to as S-oligos.

A “*” is used to denote a PS modification. In this application, the terms A*, C*, U*, or G* may be used to denote an adenine, cytosine, uridine, or guanidine nucleotide, respectively, that is linked to the next (e.g., 3′) nucleotide with a PS bond.

In this application, the terms “mA*,” “mC*,” “mU*,” or “mG*” are used to denote an adenine, cytosine, uridine, or guanidine nucleotide, respectively, that has been substituted with 2′-O-Me and that is linked to the next (e.g., 3′) nucleotide with a PS bond.

The diagram below shows the substitution of S-into a non-bridging phosphate oxygen, generating a PS bond in lieu of a phosphodiester bond:

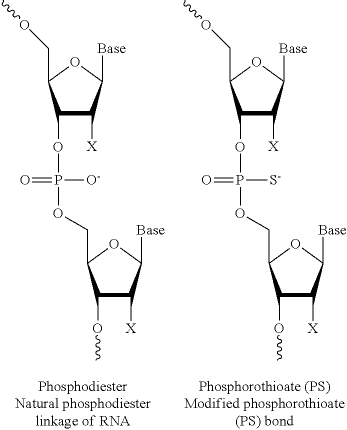

In some embodiments, guide RNAs are modified with sequence substitutions that do not comprise chemical modifications. In some embodiments, modified guide RNAs are engineered with G-C pairings (e.g., in lower or upper stem regions) that are not found in the parental guide RNA sequence. In some embodiments, modified guide RNAs are engineered with G-U mismatches (“GU wobbles” or mismatch pairings) that are not found in the parental guide RNA sequence.

Abasic nucleotides refer to those which lack nitrogenous bases. The figure below depicts an oligonucleotide with an abasic (also known as apurinic) site that lacks a base. As used herein, the presence of a single abasic site is not considered to disrupt a duplex, e.g., a duplex formed between the targeting sequence of a guide RNA and a target site in the genome:

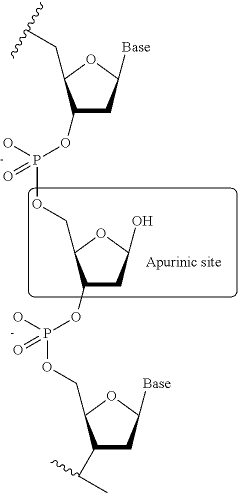

Inverted bases refer to those with linkages that are inverted from the normal 5′ to 3′ linkage (i.e., either a 5′ to 5′ linkage or a 3′ to 3′ linkage). Such inverted bases can only be present as a terminal nucleotide. In chemical synthesis methods performed 3′ to 5′, inverted bases do not have 5′ hydroxy available to grow the chain. For example:

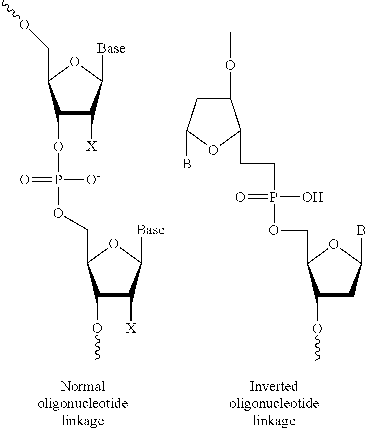

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

Exemplary bicyclic ribose analogs include locked nucleic acid (LNA), ENA, bridged nucleic acid (BNA), or another LNA-like modifications. In some instances, a bicyclic ribose analog has 2′ and 4′ positions connected through a linker. The linker can be of the formula —X—(CH2)n- where n is 1 or 2; X is O, NR, or S; and R is H or C₁-3 alkyl, e.g., methyl. Examples of bicyclic ribose analogs include LNAs comprising a 2′-O—CH2-4′ bicyclic structure (oxy-LNA) (see WO 98/39352 and WO 99/14226); 2′-NH—CH2-4′ or 2′-N(CH3)-CH2-4′ (amino-LNAs) (Singh et al., J. Org. Chem. 63:10035-10039 (1998); Singh et al., J. Org. Chem. 63:6078-6079 (1998)); and 2′-S—CH2-4′ (thio-LNA) (Singh et al., J. Org. Chem. 63:6078-6079 (1998); Kumar et al., Biorg. Med. Chem. Lett. 8:2219-2222 (1998)).

An ENA modification refers to a nucleotide comprising a 2′-O,4′-C-ethylene modification. An exemplary structure of an ENA nucleotide is shown below, in which wavy lines indicate connections to the adjacent nucleotides (or terminal positions as the case may be, with the understanding that if the 3′ terminal nucleotide is an ENA nucleotide, the 3′ position may comprise a hydroxyl rather than phosphate). For further discussion of ENA nucleotides, see, e.g., Koizumi et al., Nucleic Acids Res. 31:3267-3273 (2003).

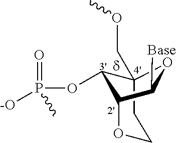

A UNA or unlocked nucleic acid modification refers to a nucleotide comprising a 2′,3′-seco-RNA modification, in which the 2′ and 3′ carbons are not bonded directly to each other. An exemplary structure of a UNA nucleotide is shown below, in which wavy lines indicate connections to the adjacent phosphates or modifications replacing phosphates (or terminal positions as the case may be). For further discussion of UNA nucleotides, see, e.g., Snead et al., Molecular Therapy 2: e103, doi: 10.1038/mtna.2013.36 (2013).

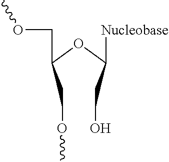

A base modification is any modification that alters the structure of a nucleobase or its bond to the backbone, including isomerization (as in pseudouridine). In some embodiments, a base modification includes inosine. In some embodiments, a modification comprises a base modification that reduces RNA endonuclease activity, e.g., by interfering with recognition of a cleavage site by an Rnase or by stabilizing an RNA structure (e.g., secondary structure) that decreases accessibility of a cleavage site to an Rnase. Exemplary base modifications that can stabilize RNA structures are pseudouridine and 5-methylcytosine. See Peacock et al., J Org Chem. 76:7295-7300 (2011). In some embodiments, a base modification can increase or decrease the melting temperature I of a nucleic acid, e.g., by increasing the hydrogen bonding in a Watson-Crick base pair, forming non-canonical base pair, or creating a mismatched base pair. Exemplary base modifications that can increase hydrogen bonding in a Watson-Crick base pair include G-clamps.

The above modifications and their equivalents are included within the scope of the embodiments described herein.

A modification at a YA site (also referred to as a YA modification) can be a modification of the internucleoside linkage, a modification of the base (pyrimidine or adenine), e.g. by chemical modification, substitution, or otherwise, or a modification of the sugar (e.g. at the 2′ position, such as 2′-O-alkyl, 2′-F, 2′-moe, 2′-F arabinose, (deoxyribose), and the like). In some embodiments, a “YA modification” is any modification that alters the structure of the dinucleotide motif to reduce RNA endonuclease activity, e.g., by interfering with recognition or cleavage of a YA site by an Rnase or by stabilizing an RNA structure (e.g., secondary structure) that decreases accessibility of a cleavage site to an RNase. See Peacock et al., J Org Chem. 76:7295-7300 (2011); Behlke, Oligonucleotides 18:305-320 (2008); Ku et al., Adv. Drug Delivery Reviews 104:16-28 (2016); Ghidini et al., Chem. Commun., 2013, 49, 9036. Peacock et al., Belhke, Ku, and Ghidini provide exemplary modifications suitable as YA modifications. Modifications known to those of skill in the art to reduce endonucleolytic degradation are encompassed. Exemplary 2′ ribose modifications that affect the 2′ hydroxyl group involved in Rnase cleavage are 2′-O-alkyl, including 2′-O-Me. Modifications such as bicyclic ribose analogs, UNA, and modified internucleoside linkages of the residues at the YA site can be YA modifications. Exemplary base modifications that can stabilize RNA structures are pseudouridine and 5-methylcytosine. In some embodiments, at least one nucleotide of the YA site is modified. In some embodiments, the pyrimidine (also called “pyrimidine position”) of the YA site comprises a modification (which includes a modification altering the internucleoside linkage immediately 3′ of the sugar of the pyrimidine, a modification of the pyrimidine base, and a modification of the ribose, e.g. at its 2′ position). In some embodiments, the adenine (also called “adenine position”) of the YA site comprises a modification (which includes a modification altering the internucleoside linkage immediately 3′ of the sugar of the pyrimidine, a modification of the pyrimidine base, and a modification of the ribose, e.g. at its 2′ position). In some embodiments, the pyrimidine and the adenine of the YA site comprise modifications. In some embodiments, the YA modification reduces RNA endonuclease activity.

The above modifications and their equivalents are included within the scope of the embodiments described herein.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification known in the art to increase stability or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide. In some embodiments, the first three nucleotides at the 5′ terminus, and the last three nucleotides at the 3′ terminus comprise an inverted abasic nucleotide.

### Modifications to Spacer Sequence

In some embodiments, the spacer sequence of a guide RNA is modified.

In some embodiments, the spacer sequence of a guide RNA comprises a 2′-O-methyl (2′-O-Me) modified nucleotide. In some embodiments, the spacer of the tgRNA comprises at least one, at least two, at least three, at least four, at least five, at least six, at least seven, at least eight, at least nine, or at least ten 2′O-Me modifications. In some embodiments, the spacer of the tgRNA comprises one, two, three, four, five, six, seven, eight, nine, ten, or eleven 2′O-Me modifications. In some embodiments, the spacer of the tgRNA comprises a 2′O-Me modification at one or more positions S1, S2, S3, S9, S10, S13, S14, S17, and S20.

In some embodiments, the spacer sequence is 20 nucleotides in length. In some embodiments, the spacer of the guide RNA comprises a modified nucleotide. In some embodiments, the spacer of the guide RNA comprises a phosphorothioate (PS) modification, a 2′-O-methyl (2′-O-Me) modified nucleotide, or a 2′-F modified nucleotide. In some embodiments, the spacer of the guide RNA comprises a phosphorothioate (PS) modification. In some embodiments, the spacer of the guide RNA comprises a 2′-O-methyl (2′-O-Me) modified nucleotide. In some embodiments, the spacer of the guide RNA comprises a phosphorothioate (PS) modification, and a 2′-F modified nucleotide.

In some embodiments, the spacer of the tgRNA comprises both DNA nucleotides and RNA nucleotides. In some embodiments, the spacer of the tgRNA comprises at least one, at least two, at least three, at least four, at least five, at least six, at least seven, at least eight, at least nine, or at least ten DNA nucleotides. In some embodiments, the spacer of the tgRNA comprises one, two, three, four, five, six, seven, eight, nine, ten, or eleven DNA nucleotides. In some embodiments, the spacer of the tgRNA comprises a DNA nucleotide at one, two, or three of positions S11, S12, and S18.

In some embodiments, the spacer of the guide RNA (or tgRNA) comprises DNA nucleotides. Without wishing to be bound by theory, the inclusion of DNA nucleotides in the spacer can prevent tgRNA degradation by RNAse H, in embodiments where the DRS region comprises DNA nucleotides. In some embodiments, the spacer of the guide RNA (or tgRNA) comprises a DNA nucleotide at at least 2, 3, 4, 5, 6, 7, 8, 9, or 10 nucleotides. In some embodiments, the spacer of the guide RNA (or tgRNA) comprises a DNA nucleotide at 1, 2, 3, 4, 5, 6, 7, 8, 9, 10, or 11 nucleotides. In some embodiments, the spacer of the guide RNA (or tgRNA) comprises a DNA nucleotide at one or more, two or more, three or more, or four or more of positions S1, S2, S3, S4, S5, S9, S10, S11, S12, S13, S17, S18, or S20, optionally wherein the spacer of the guide RNA (or tgRNA) comprises a DNA nucleotide at one or more of positions S11, S12, and S18, optionally at each of positions S11, S12, and S18. In some embodiments, the spacer of the guide RNA (or tgRNA) does not comprise DNA nucleotides. In some embodiments, the spacer of the tgRNA comprises one or more (e.g., one, two, three, four, five, six, seven, eight, nine, ten, or eleven) DNA nucleotides, and in addition comprises one or more 2′OMe or 2′F modifications. For example, a spacer containing DNA nucleotides as described herein can additionally contain 2′OMe or 2′F modifications at one or more of positions S1, S2, S3, S4, S9, S10, S11, S13, S14, S17, or S18. In one embodiment, a tgRNA spacer can comprise 2′OMe modifications at positions S1, S2, S3, S9, S10, S13, S14, S17, and S20, and DNA nucleotides at positions S11, S12, and S18. In another embodiment, a tgRNA spacer can comprise 2′OMe modifications at positions S1, S2, S3, S9, S10, S13, S14, S17, and S20, and can optionally comprise DNA nucleotides at one or more additional positions. In certain embodiments of the foregoing aspects, the spacer can be present in a tgRNA in which the DRS region contains DNA nucleotides. For example, in some embodiments, the DRS region can contain 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more DNA nucleotides. In some embodiments, each nucleotide in the DRS region contains a DNA nucleotide. In some embodiments, the nucleotides in the DRS region contain primarily DNA nucleotides, with 1, 2, 3, 4, 5, 6, 7, 8, 9, or 10 RNA nucleotides, optionally wherein the RNA nucleotides in the DRS region comprise a 2′OMe modification.

In some embodiments, the spacer of the guide RNA (e.g., tgRNA or ngRNA) comprises a modified nucleotide at one or more of the first five nucleotides at the 5′ end. In some embodiments, the spacer of the guide RNA (e.g., tgRNA or ngRNA) comprises a modified nucleotide at one or more of the first three nucleotides at the 5′ end of the spacer.

The scaffold sequences and chemical modification patterns may improve the activity of the sgRNA and the sgRNA/Spy Cas9, to cleave target DNA. There may be further considerations in selecting nucleotides for modification (e.g., for enhancing potency, half-life, stability, etc.). For example, as described herein, sgRNA nucleotides having a 2′-OH ribose moiety within hydrogen bonding distance of SpyCas9 amino acid residues may interact with SpyCas9 at certain conformation states of the sgRNA/Cas9 complex, thus rendering these nucleotides less suitable, in certain embodiments, for chemical modification at the 2′-OH ribose moiety. Where modification at one or more of these nucleotides is nonetheless desired, a 2′F may be more suitable than a 2′OMe modification, due to the relatively compact size of 2′-F relative to 2′-OMe.

Accordingly, in certain embodiments, positions S4, S5, S6, S7, S8, S9, S10, S11, S12, S13, S14, S15, S16, S17, S18, S19, and S20 comprise unmodified nucleotides. In certain embodiments, positions S5, S6, S7, S8, S12, S15, S16, S19, and S20 comprise unmodified nucleotides. In certain embodiments, positions S5, S6, S8, S12, S15, S16, S19, and S20 comprise unmodified nucleotides. In some embodiments, the spacer of the guide RNA (e.g., tgRNA or ngRNA) does not comprise a modification at one or more of positions S4, S5, S6, S7, S8, S12, S15, S16, S19, and S20. In some embodiments, the spacer of the guide RNA (e.g., tgRNA or ngRNA) does not comprise a modification at one or more of positions S6, S7, S8, S15, S16, and S19. In some embodiments, the spacer of the guide RNA (e.g., tgRNA or ngRNA) does not comprise a modification at one or more of positions S5, S6, S7, S8, S12, S15, S16, S19, and S20. In some embodiments, the spacer of the guide RNA (e.g., tgRNA or ngRNA) does not comprise a modification at one or more of positions S5, S6, S8, S12, S15, S16, S19, and S20. In some embodiments, the spacer of the guide RNA does not comprise a modification at one or more of positions S4, S5, S6, S7, S8, S11, S12, S15, S16, S18, and S19.

In some embodiments, the spacer of the guide RNA (e.g., tgRNA or ngRNA) comprises a modification at one or more of positions S4, S5, S9, S10, S11, S12, S13, S14, S17, S18, and S20. In some embodiments, the spacer of the guide RNA comprises a modification at one or more of positions S4, S9, S10, S11, S13, S14, S17, and S18. In some embodiments, the spacer of the guide RNA comprises 3 PS linkages between the 4 consecutive 5′ terminal nucleotides of the spacer. In some embodiments, the spacer of the guide RNA comprises a PS linkage between positions S7 and S8.

In some embodiments, the spacer of the guide RNA (e.g., tgRNA or ngRNA) comprises a 2′-O-Me modified nucleotide or a 2′-F modified nucleotide in at least 2, 3, 4, 5, 6, 7, 8, 9, or 10 nucleotides. In some embodiments, the spacer of the guide RNA comprises 2′-O-Me modified nucleotides at each of the first three nucleotides (S1, S2, and S3) at the 5′ end of the spacer. In some embodiments, the spacer of the guide RNA comprises 2′-F modified nucleotides at one or more of positions S9, S10, S11, S13, S14, S17, and S18. In some embodiments, the spacer of the guide RNA comprises a 2′-O-Me modified nucleotide at position S4 and 2′-F modified nucleotides at each of positions S9, S10, S11, S13, S14, S17, and S18. In some embodiments, the spacer of the guide RNA comprises a 2′-O-Me modified nucleotide at position S4, 2′-F modified nucleotides at each of positions S9, S10, S11, S13, S14, S17, and S18, and a PS linkage between positions S7 and S8.

In some embodiments, the spacer of the guide RNA (e.g., tgRNA or ngRNA) comprises 2′-O-Me modified nucleotides at one or more of positions S4, S5, S9, S10, S11, S12, S13, S14, S17, S18, and S20. In some embodiments, the spacer of the guide RNA comprises 2′-O-Me modified nucleotides at one or more of positions S4, S5, S9, S10, S13, S14, S17 and S20. In some embodiments, the spacer of the guide RNA comprises 2′-O-Me modified nucleotides at each of positions S9, S10, S13, S14, S17, and S20. In some embodiments, the spacer of the guide RNA comprises 2′-O-Me modified nucleotides at each of positions S9, S10, S17, and S20 and DNA nucleotides at each of positions S11, S12, and S18.

In some embodiments, (1) the spacer of the guide RNA (e.g., tgRNA or ngRNA) comprises 2′-O-Me modified nucleotides at each of S1, S2, S3, S9, S10, S13, S14, S17, and S20; and (b) the spacer of the guide RNA does not comprise a modification at each of S4, S5, S6, S7, S8, S11, S12, S15, S16, S18, and S19. In some embodiments, (a) the spacer of the guide RNA comprises 2′-O-Me modified nucleotides at each of S1, S2, S3, S9, S10, S13, S14, S17, and S20; (b) the spacer of the guide RNA comprises DNA nucleotides at each of positions S11, S12, and S18; and (c) the spacer of the guide RNA does not comprise a modification at each of S4, S5, S6, S7, S8, S15, S16, and S19.

In certain embodiments, the sgRNA (e.g., tgRNA or ngRNA) comprises a modified nucleotide at one or more of positions S1, S2, and S3; and comprises an unmodified nucleotide at one or more positions S4, S5, S6, S7, S8, S9, S10, S11, S12, S13, S14, S15, S16, S17, S18, S19, and S20. In certain embodiments, the sgRNA comprises a modified nucleotide at positions S1, S2, and S3; and comprises an unmodified nucleotide at positions S4, S5, S6, S7, S8, S9, S10, S11, S12, S13, S14, S15, S16, S17, S18, S19, and S20.

In certain embodiments, the sgRNA (e.g., tgRNA or ngRNA) comprises a modified nucleotide at one or more of positions S1, S2, S3, S4, S9, S10, S11, S13, S14, S17, and S18; and comprises an unmodified nucleotide at one or more of positions S5, S6, S7, S8, S12, S15, S16, S19, and S20. In certain embodiments, the sgRNA comprises a modified nucleotide at positions S1, S2, S3, S4, S9, S10, S11, S13, S14, S17, and S18; and comprises an unmodified nucleotide at positions S5, S6, S7, S8, S12, S15, S16, S19, and S20.

In certain embodiments, the sgRNA (e.g., tgRNA or ngRNA) comprises a modified nucleotide at one or more of positions S1, S2, S3, S4, S7, S9, S10, S11, S13, S14, S17, and S18; and comprises an unmodified nucleotide at one or more of positions S5, S6, S8, S12, S15, S16, S19, and S20. In certain embodiments, the sgRNA comprises a modified nucleotide at positions S1, S2, S3, S4, S7, S9, S10, S11, S13, S14, S17, and S18; and comprises an unmodified nucleotide at positions S5, S6, S8, S12, S15, S16, S19, and S20.

In some embodiments, (1) the spacer of the guide RNA (e.g., tgRNA or ngRNA) comprises 2′-O-Me modified nucleotides at each of S1, S2, S3, and S4; (2) the spacer of the guide RNA (e.g., tgRNA or ngRNA) comprises 2′-F modified nucleotides at each of positions S9, S10, S11, S13, S14, S17, and S18; and (3) the spacer of the guide RNA does not comprise a modification at each of positions S5, S6, S7, S8, S12, S15, S16, S19, and S20. In some embodiments, (1) the spacer of the guide RNA comprises 2′-O-Me modified nucleotides at each of S1, S2, S3, and S4; (2) the spacer of the guide RNA comprises 2′-F modified nucleotides at each of positions S9, S10, S11, S13, S14, S17, and S18; (3) the spacer of the guide RNA does not comprise a modification at each of positions S5, S6, S8, S12, S15, S16, S19, and S20; and (4) the spacer of the guide RNA comprises a PS linkage between positions S7 and S8.

### Modifications to Terminal Nucleotides

In some embodiments, the 5′ or 3′ terminus region of a guide RNA is modified.

### 3′ Terminus Region Modifications

In some embodiments, the terminal (i.e., last) 1, 2, 3, or 4 nucleotides in the 3′ terminus region are modified. Throughout, this modification may be referred to as a “3′ end modification”. In some embodiments, the terminal (i.e., last) 1, 2, 3, or 4 nucleotides in the 3′ terminus region comprise more than one modification. In some embodiments, at least one of the terminal (i.e., last) 1, 2, 3, or 4 nucleotides in the 3′ terminus region are modified. In some embodiments, at least two of the terminal (i.e., last) 1, 2, 3, or 4 nucleotides in the 3′ terminus region are modified. In some embodiments, at least three of the terminal (i.e., last) 1, 2, 3, or 4 nucleotides in the 3′ terminus region are modified. In some embodiments, the modification comprises a PS linkage. In some embodiments, the modification to the 3′ terminus region is a 3′ protective end modification. In some embodiments, the 3′ end modification comprises a 3′ protective end modification. It is understood that the 3′ end of a tgRNA may be the 3′ end of a guide RNA scaffold when the 3′ extension is not covalently linked to the 3′ extension. The 3′ end of a tgRNA may be the 3′ end of the 3′ extension, which may be the 3′ end of the DRS or the 3′ end of an aptamer, when present. When the 3′ extension is not covalently linked to the guide RNA scaffold, the 3′ end of both the guide RNA scaffold and the 3′ extension may be modified.

In some embodiments, the 3′ end modification comprises a modified nucleotide selected from 2′-O-methyl (2′-O-Me) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof.

In some embodiments, the 3′ end modification comprises or further comprises a 2′-O-methyl (2′-O-Me) modified nucleotide.

In some embodiments, the 3′ end modification comprises or further comprises a phosphorothioate (PS) linkage between nucleotides.

In some embodiments, the 3′ end modification comprises or further comprises a modification of any one or more of the last 4, 3, 2, or 1 nucleotides.

In some embodiments, the 3′ end modification comprises or further comprises any one or more of the following: a phosphorothioate (PS) linkage between nucleotides, a 2′-O-Me modified nucleotide, a 2′-O-moe modified nucleotide, a 2′-F modified nucleotide, an inverted abasic modified nucleotide.

In some embodiments, the 3′ end modification comprises or further comprises 1, 2, 3, or 4 PS linkages between nucleotides.

In some embodiments, the 3′ end modification comprises or further comprises at least one 2′-O-Me, 2′-O-moe, inverted abasic, or 2′-F modified nucleotide.

In some embodiments, the 3′ end modification comprises or further comprises one PS linkage, wherein the linkage is between the last and second to last nucleotide. In some embodiments, the 3′ end modification comprises or further comprises two PS linkages between the last three nucleotides. In some embodiments, the 3′ end modification comprises or further comprises four PS linkages between the last four nucleotides.

In some embodiments, the 3′ end modification comprises or further comprises a modification of one or more of the last 1-4 nucleotides, wherein the modification is one or more of a PS linkage, 2′-Ome, 2′-O-moe, or 2′-F; or a terminal inverted abasic nucleotide.

In some embodiments, the 3′ end modification comprises or further comprises a modification to the last nucleotide with 2′-Ome, 2′-O-moe, 2′-F, or combinations thereof, and an optionally one or two PS linkages to the next nucleotide or the first nucleotide of the 3′ tail.

In certain embodiments, the 3′ end modification comprises a 2′-O-Me modified nucleotide and a PS linkage. In some embodiments, the 3′ end modification comprises the same number of 2′-O-Me modified nucleotides and PS linkages. In some embodiments, the 3′ end modification comprises one more 2′-O-Me modified nucleotides than PS linkages. In some embodiments, the 3′ end modification comprises one fewer 2′-O-Me modified nucleotides than PS linkages. In certain embodiments, the 3′ end modification comprises 4 2′-O-Me modified nucleotides. In certain embodiments, the 3′ end modification comprises 3 2′-O-Me modified nucleotides.

In some embodiments, when the modified guide RNA comprises a 3′ extension that is covalently linked to the 3′ end of the scaffold sequence, the modified guide RNA does not comprise a modified residue at or near the 3′ end of the scaffold sequence.

### 3′ Tail

In some embodiments, the guide RNA comprising a 3′ end modification comprises or further comprises a 3′ tail, wherein the 3′ tail comprises a modification of any one or more of the nucleotides present in the 3′ tail. In some embodiments, the 3′ tail is fully modified. In some embodiments, the 3′ tail comprises 1, 2, 3, 1-2, or 1-4 nucleotides, optionally where any one or more of these nucleotides are modified.

In some embodiments, the guide RNA comprises a 3′ terminus comprising a 3′ tail, which follows and is 3′ of the conserved portion of a guide RNA. In some embodiments, the 3′ tail comprises 1-4 nucleotides, e.g., 1-2 nucleotides. In some embodiments, the 3′ tail comprises 1 nucleotide. In some embodiments, the 3′ tail comprises 2 nucleotides. In some embodiments, the 3′ tail comprises 3 nucleotides. In some embodiments, the tail terminates with a nucleotide comprising a uridine nucleotide or a modified uridine nucleotide. In some embodiments, the 3′ tail is 1 nucleotide in length and is a nucleotide comprising a uridine nucleotide or a modified uridine nucleotide. In some embodiments, the 3′ nucleotide of the guide RNA is a nucleotide comprising a uridine nucleotide or a modified uridine nucleotide.

In some embodiments, the 3′ tail comprising 1-4 nucleotides, e.g., 1-2 nucleotides, is at the 3′ end of the conserved portion of a guide RNA. In some embodiments, the 3′ tail comprises 1~4 nucleotides, e.g., 1-2 nucleotides is at the 3′ end of the 3′ extension.

In some embodiments, the 3′ tail comprises or further comprises a protective end modification selected from a phosphorothioate (PS) linkage between nucleotides, a 2′-Ome modified nucleotide, a 2′-O-moe modified nucleotide, a 2′-F modified nucleotide, an inverted abasic modified nucleotide as a terminal nucleotide.

In some embodiments, the 3′ tail comprises or further comprises a phosphorothioate (PS) linkages between nucleotides. In some embodiments, the 3′ tail comprises or further comprises a 2′-Ome modified nucleotide. In some embodiments, the 3′ tail comprises or further comprises a combination of a phosphorothioate (PS) linkage between nucleotides, and a 2′-Ome modified nucleotide.

In some embodiments, the guide RNA does not comprise a 3′ tail.

### 5′ Terminus Region Modification

In some embodiments, the 5′ terminus region is modified, for example, the first 1, 2, 3, or 4 nucleotides of the guide RNA are modified. Throughout, this modification may be referred to as a “5′ end modification”. In some embodiments, one or more of the first 1, 2, 3, or 4 nucleotides of the 5′ terminus region comprise more than one modification. In some embodiments, the first nucleotide at the 5′ terminus comprises more than one chemical modification. In some embodiments, the 5′ end modification is a 5′ protective end modification.

In some embodiments, both the 5′ and 3′ terminus regions (e.g., ends) of the guide RNA are modified. In some embodiments, only the 5′ terminus region of the guide RNA is modified. In some embodiments, only the 3′ terminus region (plus or minus a 3′ tail) of the conserved portion of a guide RNA is modified.

In some embodiments, the guide RNA comprises modifications at 1, 2, 3, or 4 of the first 4 nucleotides at a 5′ terminus region of the guide RNA. In some embodiments, the guide RNA comprises modifications at 1, 2, 3, or 4 of the 4 terminal nucleotides at a 3′ terminus region. In some embodiments, 2, 3, or 4 of the first 4 nucleotides at the 5′ terminus region, or 2, 3, or 4 of the terminal 4 nucleotides at the 3′ terminus region are modified. In some embodiments, 2, 3, or 4 of the first 4 nucleotides at the 5′ terminus region are linked with phosphorothioate (PS) bonds.

In some embodiments, the modification to the 5′ terminus or 3′ terminus comprises a 2′-O-methyl(2′-O-Me), 2′-O-(2-methoxyethyl) (2′-O-moe) modification, or a 2′-fluoro (2′-F) modification, or a phosphorothioate (PS) linkage between nucleotides. In some embodiments, the modification comprises an inverted abasic terminal nucleotide. In some embodiments, the modification comprises a protective end modification. In some embodiments, the modification comprises a more than one modification. In some embodiments, the modifications comprise 2′-O-Me and PS.

In some embodiments, the guide RNA comprises a phosphorothioate (PS) linkage between the first 1, 2, 3, or 4nucleotides at the 5′ terminus. In some embodiments, the guide RNA comprises a PS linkage between the last 1, 2, 3, or 4 nucleotides at the 3′ terminus. In some embodiments, the guide RNA comprises a PS linkage between both the last 1, 2, 3, or 4 nucleotides at the 3′ terminus and the 1, 2, 3, or 4 nucleotides from the 5′ end of the 5′ terminus. In some embodiments, in addition to PS linkages, the 5′ and 3′ terminal nucleotides may comprise 2′-O-Me modified nucleotides.

In some embodiments, the guide RNA comprises a 5′ end modification, e.g., wherein the first nucleotide of the guide region is modified. In some embodiments, the guide RNA comprises a 5′ end modification, wherein the first nucleotide of the guide region comprises a 5′ protective end modification.

In some embodiments, the 5′ end modification comprises a modified nucleotide selected from 2′-O-methyl (2′-O-Me) modified nucleotide, 2′-O-(2-methoxyethyl) (2′-O-moe) modified nucleotide, a 2′-fluoro (2′-F) modified nucleotide, or a phosphorothioate (PS) linkage between nucleotides; or an inverted abasic modified terminal nucleotide.

In some embodiments, the 5′ end modification comprises or further comprises a 2′-O-methyl (2′-O-Me) modified nucleotide.

In some embodiments, the 5′ end modification comprises or further comprises a phosphorothioate (PS) linkage between nucleotides.

In some embodiments, the 5′ end modification comprises or further comprises a modification of any one or more of nucleotides 1~4 of the guide region of a guide RNA. In some embodiments, the 5′ end modification comprises or further comprises one modified nucleotide. In some embodiments, the 5′ end modification comprises or further comprises two modified nucleotides. In some embodiments, the 5′ end modification comprises or further comprises three modified nucleotides. In some embodiments, the 5′ end modification comprises or further comprises four modified nucleotides.

In some embodiments, the 5′ end modification comprises or further comprises a modification of 1-4 nucleotides from the 5′ end. In some embodiments, the 5′ end modification comprises or further comprises a modification of the most 5′ terminal nucleotide. In some embodiments, the 5′ end modification comprises or further comprises a modification of the 3 most 5′ terminal nucleotides.

In some embodiments, the 5′ end modification comprises or further comprises a phosphorothioate (PS) linkage between nucleotides, or a 2′-O-Me modified nucleotide, or a 2′-O-moe modified nucleotide, or a 2′-F modified nucleotide, or an inverted abasic modified nucleotide. In some embodiments, the 5′ end modification comprises or further comprises a phosphorothioate (PS) linkage between nucleotides and a 2′-O-Me modified nucleotide.

### Repeat-Anti-Repeat Region Modifications

In some embodiments, a guide RNA is provided comprising a repeat-anti-repeat region modification, wherein the repeat-anti-repeat region modification comprises a modification of at least 1, 2, 3, 4, 5, 6, 7, 8, 9, 10, 11, or 12, or all of the nucleotides in the upper stem of the repeat-anti-repeat region.

In some embodiments, a guide RNA is provided comprising a repeat-anti-repeat region modification, wherein the repeat-anti-repeat region modification comprises a modification of all of the upper stem region, when an upper stem region is present. In some embodiments, no upper stem region is present, e.g., the upper stem region is substituted with an internal linker in the bulge region.

In some embodiments, a guide RNA is provided comprising a repeat-anti-repeat region modification, wherein the upper stem modification comprises a 2′-Ome modified nucleotide. In some embodiments, a guide RNA is provided comprising a repeat-anti-repeat region modification, wherein the upper stem modification comprises a 2′-Ome modified nucleotide, a 2′-O-moe modified nucleotide, or a 2′-F modified nucleotide.

In some embodiments, the guide RNA comprises a 5′ end modification and a repeat-anti-repeat region modification, e.g., an upper stem modification. In some embodiments, the guide RNA comprises a 3′ end modification and a repeat-anti-repeat region modification, e.g., an upper stem modification. In some embodiments, the guide RNA comprises a 5′ end modification, a 3′ end modification, and a repeat-anti-repeat region modification, e.g., an upper stem modification.

### Hairpin Modifications

In some embodiments, the guide RNA comprises a modification in the hairpin region. In some embodiments, the hairpin region modification comprises at least one modified nucleotide selected from a 2′-O-methyl (2′-Ome) modified nucleotide, a 2′-fluoro (2′-F) modified nucleotide.

In some embodiments, the hairpin region modification is in the hairpin 1 region. In some embodiments, the hairpin region modification is in the hairpin 2 region. In some embodiments, the hairpin region modification is only in the hairpin 2 region. In some embodiments, the hairpin region modification is in the hairpin 2 region at positions that could be characterized a 3′ end modification. In some embodiments, modifications are within the hairpin 1 and hairpin 2 regions, optionally wherein the “n” between hairpin 1 and 2 is also modified.

In some embodiments, the hairpin modification comprises or further comprises a 2′-O-methyl (2′-Ome) modified nucleotide.

In some embodiments, the hairpin modification comprises or further comprises a PS modified nucleotide.

In some embodiments, the hairpin region modification comprises at least one modified nucleotide selected from a 2′H modified nucleotide (DNA), PS modified nucleotide, a 2′-O-methyl (2′-O-Me) modified nucleotide, a 2′-fluoro (2′-F) modified nucleotide.

In some embodiments, the guide RNA comprises a 5′ end modification, and a modification in the hairpin region, e.g., a hairpin 2 region modification, e.g., a hairpin 2 region modification at positions that could be characterized a 3′ end modification.

In some embodiments, the guide RNA comprises a repeat-anti-repeat region modification, e.g., an upper stem modification, and a modification in the hairpin region, e.g., a hairpin 2 region modification, e.g., a hairpin 2 region modification at positions that could be characterized a 3′ end modification.

In some embodiments, the guide RNA comprises a 3′ end modification, a modification in the hairpin region, e.g., a hairpin 2 region modification, e.g., a hairpin 2 region modification at positions that could be characterized a 3′ end modification, a repeat-anti-repeat region modification, e.g., an upper stem modification, and a 5′ end modification.

### 3′ Extension

In some embodiments, the second polynucleotide of the template guide RNA is a 3′ extension. In some embodiments, the second polynucleotide of the template guide RNA is a chimeric DNA/RNA 3′ extension polynucleotide. In some embodiments, all of the nucleotides constituting the template sequence are DNA nucleotides and the DRS comprise a plurality of RNA nucleotides. In some embodiments, all of the nucleotides constituting the template sequence are DNA nucleotides and the DRS comprise a single DNA nucleotide at its 5′ end. In some embodiments all of the nucleotides constituting the template sequence are DNA nucleotides and all of the nucleotides constituting the DRS are RNA nucleotides. In some embodiments, 1-3 nucleotides at the 3′ end of the template (T1-T3), optionally only 1 nucleotide at the 3′ end of the template (T1) are RNA nucleotides, with the remaining nucleotides in the template being DNA nucleotides. In some embodiments, 1-7 nucleotides at the 5′ end of the DRS (nucleotides D1-D7) are DNA nucleotides, with the remaining nucleotides in the DRS being RNA nucleotides.

In some embodiments, the template sequence comprises a modified nucleotide; or the DRS comprises a modified nucleotide. In some embodiments, the one or more modified nucleotides for use at an RNA position is selected from the group: 2′-O-Me; 2′-F; an amine; an alkoxy; an LNA; methoxyethyl; a halo; PS linkages; or an amine. In certain embodiments, the modified nucleotide is a 2′-O-Me modified nucleotide.

Provided herein are 3′ extensions comprising a template sequence for the DNA-dependent DNA polymerase, a DNA-dependent DNA polymerase recruiting sequence (DRS), and, optionally, one or more affinity tags such as a small molecule or an aptamer. In some embodiments, the 3′ extension comprises a template sequence positioned 5′ of a DRS. In some embodiments, the 3′ extension comprises an affinity tag in addition to the template sequence positioned 5′ of a DRS. In some embodiments, the template sequence, DRS, and affinity tag are linked in the following orientation: 5′-affinity tag-template sequence-DRS-3′. In some embodiments, the template sequence, DRS, and affinity tag are linked in the following orientation: 5′-template sequence-DRS-affinity tag-3′. In some embodiments, the 3′ extension comprises two affinity tags and the template sequence, DRS, and affinity tag are linked in the following orientation: 5′-first affinity tag-template sequence-DRS-second affinity tag-3′. In some embodiments, the first and second affinity tags are the same. In some embodiments, the first and second affinity tags are different.

### Template Sequence

In some embodiments, the present disclosure provides for a 3′ extension comprising a template sequence. The template sequence provides a nucleotide sequence that is different from an endogenous nucleotide sequence prior to template-based genome editing. That is, the template sequence may comprise one or more of an insertion, deletion, or substitution (e.g., a transition or transversion) relative to the endogenous nucleotide sequence prior to template-based genome editing. After template-based genome editing and subsequent DNA repair, the endogenous nucleotide sequence may include the insertion, deletion, or substitution encoded by the template sequence.

Because of their ability to facilitate precise, templated editing at a target sequence in a genomic locus, template sequences are of great interest for correcting mutations associated with genetic diseases. In some embodiments, the template can direct single nucleotide insertions, (e.g., insertions of 1 nucleotide), small insertions (e.g., insertions of 1-5 nucleotides), large insertions (e.g, insertions of 5 or more nucleotides), single nucleotide deletions (e.g., deletion of 1 nucleotide), small deletions (e.g., deletion of 1-5 nucleotides), or large deletions (e.g., deletion of 5 or more nucleotides). In other embodiments, the template can direct the specific substitution of one or more nucleotides. For example, the template can, in some embodiments, direct the substitution of 1 nucleotide, 1-5 nucleotides, or 5 or more nucleotides with a desired sequence. In some embodiments, the template encodes a transition (i.e., replacement of a purine with another purine, or replacement of a pyrimidine with another pyrimidine). In some embodiments, the template encodes a transversion (i.e., replacement of a purine with a pyrimidine, or replacement of a pyrimidine with a purine).

The ability to introduce precise edits into a genomic sequence has numerous applications, non-limiting examples of which are described herein. For example, in certain embodiments, a template sequence comprising a single nucleotide insertion or deletion relative to the target sequence may be used to restore an open reading frame in an allele of a protein-coding gene harboring a frameshift mutation. In certain embodiments, a template sequence comprising a single nucleotide insertion, deletion, or substitution relative to a target sequence may be used to correct a disease-causing mutation. For example, a non-sense mutation can be corrected by removing a premature stop codon. A point mutation can be corrected by substituting a correct nucleotide for an altered or mutant nucleotide, thereby restoring the correct coding sequence of a gene of interest, or to restore a wildtype start or stop codon in a protein-coding gene. In certain embodiments, template sequences comprising an insertion relative to a target sequence may be used to restore the full-length coding sequence in an allele of a protein-coding gene comprising a large deletion. In certain embodiments, template sequences comprising a deletion relative to a target sequence may be used to restore the full-length coding sequence in an allele of a protein-coding gene comprising a repeat expansion. In certain embodiments, template sequences comprising one or more substitutions relative to a target sequence may be used to make one or more nonsynonymous substitutions in an allele of a protein-coding gene. In all of these exemplary cases, correction of the disease-associated mutation may lead to partial or full restoration of wildtype protein expression and activity.

In other embodiments, the template may be used to introduce a stop codon at a precise location within a genomic sequence.

In certain embodiments, template sequences comprising one or more insertions, deletions, or substitutions relative to a target sequence may be used to make one or more edits in a noncoding region of the genome. In certain non-limiting embodiments, the template sequence may comprise one or more insertions, deletions, or substitutions relative to one or more DNA or RNA regulatory elements, e.g., an enhancer, promoter, silencer, DNA boundary element, splicing enhancer, splicing silencer, or polyadenylation signal.

It is to be understood that the design of the template sequence, including but not limited to features such as the length, relative position of the edit within the template sequence, and number and position of DNA nucleotides varies based on the target sequence and desired edit. For example, the distance between the PAM and the site of the desired insertion, deletion, or substitution influences the position of the insertion, deletion, or substitution (relative to the genomic DNA sequence) within the template sequence. For example, if the desired edit falls between the nick site and the PAM (i.e., within the 3 nucleotides 5′ of the PAM), then the template sequence can comprise an insertion, deletion, or substitution within the last 3 nucleotides at its 3′ end (i.e., the last three nucleotides before the boundary between the template sequence and the DRS). Alternatively, if the desired edit falls downstream of the PAM, then the insertion, deletion, or substitution can be positioned farther away from the boundary between the template and the DRS. In addition, the number of nucleotides at the 5′ end of the template sequence that are complementary to the genomic DNA sequence can be varied based on factors including but not limited to the number of edited nucleotides (e.g., the number of substituted nucleotides or the length of an insertion or deletion) and the G/C and A/T content of the genomic DNA.

The template sequence comprises DNA nucleotides wherein the plurality of nucleotides in the template sequence are DNA nucleotides. In some embodiments, all nucleotides of the template are preferably DNA nucleotides (dNTPs). In some embodiments, the template of the tgRNA comprises both DNA nucleotides and RNA nucleotides. In some embodiments, 1-3 nucleotides at the 3′ end of the template (T1-T3), optionally only 1 RNA nucleotide at the 3′ end of the template (T1) are RNA nucleotides. In some embodiments, 1-3 nucleotides at the 3′ end of the template (T1-T3), optionally two nucleotides (e.g., at positions T1 and T2) or only one nucleotide at the 3′ end of the template (T1) are RNA nucleotides. In some embodiments, 1-3 nucleotides at the 3′ end of the template (T1-T3) are RNA nucleotides. In some embodiments, two nucleotides at the 3′ end of the template (e.g., at positions T1 and T2, at positions T1 and T3, or at positions T2 and T3). In some embodiments, only one nucleotide at the 3′ end of the template (e.g., T1) is a RNA nucleotide.

In some embodiments, the template sequence comprises one or more modified nucleotides. In some embodiments, the modified nucleotide is selected from the group: 2′-O-Me; 2′-F; an amine; an alkoxy; an LNA; methoxyethyl; a halo; PS linkages; or an amine. In some embodiments, the modified nucleotide is 2′-O-Me. In some embodiments, the modification in the template comprises a PS modification.

In some embodiments, the template sequence is at least 5 nucleotides in length. In some embodiments, the template sequence is at least 10 nucleotides in length. In some embodiments, the template sequence is 5-100 nucleotides in length. In some embodiments, the template sequence is 10-100 nucleotides in length. In some embodiments, the template sequence is 10-500 nucleotides in length. In some embodiments, the template sequence is 10-80 nucleotides in length. In some embodiments, the template sequence is at least 6, 7, 8, 9, or 10 nucleotides in length. In some embodiments, the template sequence is at least 5, 10, 15, 20, 25, 30, 35, 40, 45, 50, 55, 60, 65, 70, 75, 80, 85, 90, 95, or 100 nucleotides in length. In some embodiments, the template sequence is at least 50, 100, 150, 200, 250, 300, 350, 400, 450, or 500 nucleotides in length.

In some embodiments, the template sequence is 10-20 nucleotides in length. In some embodiments, the template sequence is 12-20 nucleotides in length. In some embodiments, the template sequence is 14, 15, 16, 17, or 18 nucleotides in length. In some embodiments, the template sequence is 14 nucleotides in length. In some embodiments, the template sequence is 15 nucleotides in length. In some embodiments, the template sequence is 16 nucleotides in length. In some embodiments, the template sequence is 17 nucleotides in length.

In some embodiments, the template sequence is 10-10,000 nucleotides in length. In some embodiments, the template sequence is at least 10, 1000, 2000, 3000, 4000, 5000, 6000, 7000, 8000, 9000, or 10,000 nucleotides in length. In some embodiments, the template sequence is at least 10-1000, 1000-2000, 2000-3000, 3000-4000, 4000-5000, 5000-6000, 6000-7000, 7000-8000, 8000-9000, or 9000-10,000 nucleotides in length. In some embodiments, the template sequence is at least 10-10,000, 1000-10,000, 2000-10,000, 3000-10,000, 4000-10,000, 5000-10,000, 6000-10,000, 7000-10,000, 8-10,000, or 9-10,000 nucleotides in length. In some embodiments, the template sequence is at least 10-1000, 10-2000, 10-3000, 10-4000, 10-5000, 10-6000, 10-7000, 10-8000, 10-9000, or 10-10,000 nucleotides in length.

In some embodiments, the template sequence comprises a mismatch relative to the genomic DNA sequence. In some embodiments, the mismatch directs a template-based edit in the genomic DNA within nucleotides T1-T25, optionally within nucleotides T1-T15 of the template. In some embodiments, the mismatch directs a template-based edit in the genomic DNA within nucleotides T1-T10 of the template. In some embodiments, the template sequence comprises a mismatch to the genomic sequence at at least one of positions T1-T6, optionally at at least one of positions T5-T6 5′ of the template junction with the DRS, wherein the template introduces a change in the amino acid sequence encoded by the genomic sequence

In some embodiments, the template sequence further comprises at least 10 nucleotides 5′ of a sequence to be edited by template-based genome editing, wherein the at least 10 nucleotides are complementary to the second region of the target nucleic acid. In some embodiments, the template sequence comprises a sequence of at least 10 nucleotides in length that is complementary to the second region of the target nucleic acid. In some embodiments, the template sequence comprises at least 10 nucleotides that are complementary to the second region of the target nucleic acid. In some embodiments, at least 3 consecutive nucleotides in the template sequence are fully complementary to the corresponding genomic sequence of a second region of the target nucleic acid. In some embodiments, the template sequence comprises at least 5, 10, 15, or 20nucleotides that are fully complementary to the second region of the target nucleic acid.

In some embodiments, the nucleotides that are complementary to the second region of the DNA duplex target nucleic acid are positioned 5′ of a templated insertion or deletion. In some embodiments, the nucleotides that are complementary to the second region of the DNA-duplex target nucleic acid are positioned 5′ of a substitution. In some embodiments, the nucleotides that are complementary to the second region of the target nucleic acid are fully complementary to the second region of the target nucleic acid.

The template sequence provides a nucleotide sequence that is different from an endogenous nucleotide sequence prior to template-based genome editing. In some embodiments, the template sequence comprises a sequence for directing a template-based insertion, deletion, or substitution relative to an endogenous nucleotide sequence prior to template-based genome editing. In some embodiments, the template sequence comprises an insertion and a substitution. In some embodiments, the template sequence comprises a deletion and a substitution. In some embodiments, the template sequence comprises an insertion and a deletion. In some embodiments, the template sequence comprises an insertion relative to an endogenous nucleotide sequence prior to template-based genome editing. In some embodiments, the template sequence comprises a deletion relative to an endogenous nucleotide sequence prior to template-based genome editing. In some embodiments, the template sequence comprises a substitution relative to an endogenous nucleotide sequence prior to template-based genome editing. In some embodiments, the substitution is a transition. In some embodiments, the substitution is a transversion.

In some embodiments, the template sequence comprises an insertion sequence relative to the corresponding genomic sequence. In some embodiments, the template sequence comprises an insertion sequence of at least one nucleotide. In some embodiments, the template sequence comprises a sequence for directing at least two non-adjacent insertions relative to the corresponding genomic sequence, further wherein each insertion comprises at least one nucleotide.

In some embodiments, the template sequence comprises an insertion sequence of 1-5, 1-10, 1-15, 1-20, 1-30, 1-40, 1-50, 1-60, 1-70, 1-80, 1-90, or 1-100 nucleotides in length. In some embodiments, the template sequence comprises an insertion sequence of at least 1, 2, 3, 4, 5, 6, 7, 8, 9, or 10 nucleotides in length. In some embodiments, the template sequence comprises an insertion sequence of at least 1, 5, 10, 15, 20, 25, 30, 35, 40, 45, 50, 55, 60, 65, 70, 75, 80, 85, 90, 95, or 100 nucleotides in length. In some embodiments, the template sequence comprises an insertion sequence of 1-100, 25-75, 5-100, 50-100, or 100-200 nucleotides in length.

In some embodiments, the template sequence comprises an insertion sequence of at least 3, 10, 15, 20, 30, 40, or 50 nucleotides in length. In some embodiments, the template sequence comprises an insertion sequence of up to 500, 1000, 3500, or 5000 nucleotides in length, optionally up to 10,000 nucleotides in length. In some embodiments, the template sequence comprises a nucleotide sequence 5′ of the insertion sequence that is at least 5 nucleotides, optionally at least 10, 20, 30, 40, or 50 nucleotides complementary to the genomic sequence of the non-target strand of the DNA duplex target nucleic acid.

In some embodiments, the template sequence comprises an insertion sequence of at least 100 nucleotides in length. In some embodiments, the template sequence comprises an insertion sequence of at least 100, 200, 300, 400, 500, 600, 700, 800, 900, or 1000 nucleotides in length. In some embodiments, the template sequence comprises an insertion sequence of 1-1000 nucleotides in length. In some embodiments, the template sequence comprises an insertion sequence of 1-10,000, 10-5,000, or 10-1000 nucleotides in length.

In some embodiments, the template region 5′ of the insertion, substitution, or deletion can comprise at least 5, at least 10, at least 15, at least 20, at least 25, at least 30, at least 35, at least 40, at least 45, at least 50 or more nucleotides complementary to the genomic DNA sequence. In some embodiments, the template region 5′ of the insertion, substitution, or deletion can comprise 5-40 nucleotides complementary to the genomic DNA sequence.

In some embodiments, the insertion or at least two non-adjacent insertions are positioned at or near the 5′ end of the template. In some embodiments, the 5′ terminal nucleotide of the template sequence does not comprise one or more inserted nucleotides. In some embodiments, at least 1, 2, 3, 4, 5, 6, 7, 8, 9, or 10 consecutive nucleotides at the 5′ end of the template sequence do not comprise one or more inserted nucleotides. In some embodiments, at least 1, 2, 3, 4, 5, 6, 7, 8, 9, 10, 11, 12, 13, 14, 15, 16, 17, 18, 19, 20, 21, 22, 23, 24, or 25 consecutive nucleotides at the 5′ end of the template sequence do not comprise one or more inserted nucleotides. In some embodiments, the insertion or at least two non-adjacent insertions are positioned at or near the 3′ end of the template (i.e., the boundary between the template and the DRS). In some embodiments, one or more of the last 1, 2, or 3 nucleotides at the 3′ end of the template comprise an insertion or at least two non-adjacent insertions. In some embodiments, the 3′ terminal nucleotide of the template comprises one or more inserted nucleotide. In some embodiments, the 3′ terminal nucleotide of the template sequence does not comprise one or more inserted nucleotides. In some embodiments, the insertion or at least two non-adjacent insertions are positioned near the middle of the template (i.e., the template comprises similar numbers of non-inserted nucleotides 5′ and 3′ of the insertion or at least two non-adjacent insertions).

In some embodiments, the template sequence can comprise an insertion of one or more nucleotides relative to the corresponding genomic sequence. In some embodiments, the inserted nucleotide or nucleotides is within positions T1-T100 (see, e.g., FIGS. 2A-2B). In some embodiments, the inserted nucleotide or nucleotides is within positions T1-T50, for example, within positions T1-T5, T5-T10, T10-15, T15-T20, T20-T25, T25-T30, T30-T35, T35-T40, T40-T45, or T45-T50.

In some embodiments, the template sequence comprises a sequence for directing a deletion relative to the corresponding genomic sequence. In some embodiments, the template sequence comprises a sequence for directing a deletion of at least one nucleotide. In some embodiments, the template sequence comprises a sequence for directing at least two non-adjacent deletions relative to the corresponding genomic sequence, further wherein each deletion comprises at least one nucleotide.

In some embodiments, the template sequence comprises a sequence for directing a deletion of 1-40 nucleotides in length. In some embodiments, the template sequence comprises a sequence for directing a deletion of at least 1, 2, 3, 4, 5, 6, 7, 8, 9, or 10 nucleotides in length. In some embodiments, the template sequence comprises a sequence for directing a deletion of at least 1, 5, 10, 15, 20, 25, 30, 35, 40, 45, 50, 55, 60, 65, 70, 75, 80, 85, 90, 95, or 100 nucleotides in length. In some embodiments, the template sequence comprises a sequence for directing a deletion of at least 100 nucleotides in length. In some embodiments, the template sequence comprises a sequence for directing a deletion of at least 100, 200, 300, 400, 500, 600, 700, 800, 900, or 1000 nucleotides in length.

In some embodiments, the template sequence comprises a sequence to direct a deletion of at least 3, 10, 15, 20, 30, 40, or 50 nucleotides in length. In some embodiments, the template sequence comprises the template sequence comprises a sequence to direct a deletion of up to 3500 nucleotides in length, optionally up to 10,000 nucleotides in length. In some embodiments, the template sequence comprises a nucleotide sequence 5′ of the sequence to direct a deletion that is at least 10 nucleotides, optionally at least 20, 30, 40, or 50 nucleotides complementary to the genomic sequence of the non-target strand of the DNA duplex target nucleic acid.

In some embodiments, the template sequence comprises a sequence for directing a deletion of up to 3500 nucleotides in length, optionally up to 10,000 nucleotides in length. In some embodiments, the template sequence comprises a sequence for directing a deletion of 1-10,000 nucleotides in length.

In some embodiments, the deletion or at least two non-adjacent deletions are positioned at or near the 5′ end of the template. In some embodiments, the 5′ terminal nucleotide of the template sequence does not comprise one or more deleted nucleotides. In some embodiments, at least 1, 2, 3, 4, 5, 6, 7, 8, 9, or 10 consecutive nucleotides at the 5′ end of the template sequence do not comprise a deleted nucleotide. In some embodiments, at least 1, 2, 3, 4, 5, 6, 7, 8, 9, 10, 11, 12, 13, 14, 15, 16, 17, 18, 19, 20, 21, 22, 23, 24, or 25 consecutive nucleotides at the 5′ end of the template sequence do not comprise a deleted nucleotide. In some embodiments, the deletion or at least two non-adjacent deletions are positioned at or near the 3′ end of the template (i.e., the boundary between the template and the DRS). In some embodiments, one or more of the last 1, 2, or 3 nucleotides at the 3′ end of the template comprise a deletion or at least two non-adjacent deletions. In some embodiments, the 3′ terminal nucleotide of the template comprises one or more deleted nucleotide. In some embodiments, the 3′ terminal nucleotide of the template sequence does not comprise one or more deleted nucleotides. In some embodiments, the deletion or at least two non-adjacent deletions are positioned near the middle of the template (i.e., the template comprises similar numbers of non-deleted nucleotides 5′ and 3′ of the deletion or at least two non-adjacent deletions).

In some embodiments, the template sequence can comprise a deletion of one or more nucleotides relative to the corresponding genomic sequence. In some embodiments, (1) the nucleotide position that corresponds to the nucleotide immediately 5′ of the genomic sequence that is to be deleted and (2) the nucleotide position that corresponds to the nucleotide immediately 3′ of the genomic sequence that is to be deleted are within positions T1-T50 (see, e.g., FIGS. 2A-2B). In some embodiments, (1) the nucleotide position that corresponds to the nucleotide immediately 5′ of the genomic sequence that is to be deleted and (2) the nucleotide position that corresponds to the nucleotide immediately 3′ of the genomic sequence that is to be deleted are within positions T1-T5, T5-T10, T10-15, T15-T20, T20-T25, T25-T30, T30-T35, T35-T40, T40-T45, or T45-T50.

In some embodiments, the insertion sequence or sequence for directing a deletion in the template sequence provides the template sequence for template-based genome editing comprising an insertion or deletion. In some embodiments, the insertion or deletion disrupts the spacing between a target sequence and a PAM.

In some embodiments, the template sequence comprises a sequence for directing a substitution relative to the corresponding genomic sequence. In some embodiments, the substitution is a transition. In some embodiments, the substitution is a transversion. In some embodiments, the template sequence comprises a sequence for directing a substitution of a single nucleotide. In some embodiments, the template sequence comprises a sequence for directing a substitution of at least two nucleotides. In some embodiments, wherein the template sequence comprises a sequence for directing a substitution of at least two nucleotides, the at least two nucleotides are consecutive. In some embodiments, wherein the template sequence comprises a sequence for directing a substitution of at least two nucleotides, the at least two nucleotides are non-consecutive. In some embodiments, the substituted nucleotide or nucleotides are positioned at or near the 5′ end of the template. In some embodiments, the 5′ terminal nucleotide of the template sequence does not comprise a substituted nucleotide. In some embodiments, at least 1, 2, 3, 4, 5, 6, 7, 8, 9, or 10 consecutive nucleotides at the 5′ end of the template sequence do not comprise a substituted nucleotide. In some embodiments, at least 1, 2, 3, 4, 5, 6, 7, 8, 9, 10, 11, 12, 13, 14, 15, 16, 17, 18, 19, 20, 21, 22, 23, 24, or 25 consecutive nucleotides at the 5′ end of the template sequence do not comprise a substituted nucleotide. In some embodiments, the substituted nucleotide or nucleotides are positioned at or near the 3′ end of the template (i.e., the boundary between the template and the DRS). In some embodiments, one or more of the last 1, 2, or 3 nucleotides at the 3′ end of the template comprise a substituted nucleotide. In some embodiments, the 3′ terminal nucleotide of the template comprises a substituted nucleotide. In some embodiments, the 3′ terminal nucleotide of the template sequence does not comprise a substituted nucleotide. In some embodiments, the substituted nucleotide or nucleotides are positioned near the middle of the template (i.e., the template comprises similar numbers of non-substituted nucleotides 5′ and 3′ of the substituted nucleotide or nucleotides).

In some embodiments, the template sequence can comprise a substitution of one or more nucleotides relative to the corresponding genomic sequence. In some embodiments, the substituted nucleotide or nucleotides is within positions T1-T50 (see, e.g., FIGS. 2A-2B). In some embodiments, the substituted nucleotide or nucleotides is within positions T1-T5, T5-T10, T10-15, T15-T20, T20-T25, T25-T30, T30-T35, T35-T40, T40-T45, or T45-T50.

In some embodiments, the template sequence comprises at least one mismatch relative to the corresponding genomic sequence, wherein the mismatches provide the template sequence for template-based genome editing comprising a substitution. In some embodiments, the template sequence comprises at least three mismatches relative to the corresponding genomic sequence, wherein the mismatches provide the template sequence for template-based genome editing comprising a substitution. In some embodiments, the template further comprises an insertion sequence or a sequence for directing a deletion. In some embodiments, the mismatches introduce a mutation into the PAM of the edited DNA sequence. In some embodiments, the mismatches introduce a mutation into a region of the target nucleic acid adjacent to the PAM.

In some embodiments, the template contains one or more silent mutation(s), in addition to the desired edit(s). Such silent mutations create alternations in the genomic sequence (e.g., substitutions) which do not alter the amino acid sequence of an encoded protein. In some embodiments, the template sequence comprises a silent mutation immediately adjacent to a desired edit. In some embodiments, the template sequence comprises two silent mutations immediately adjacent to a desired edit. In some embodiments, the template sequence comprises up to three, up to four, up to five, or more silent mutations within 10, within 20, or within 30 nucleotides of the desired edit(s). In one embodiment, the template sequence comprises up to three silent mutations within 10 nucleotides of the desired edit(s). In some embodiments, the silent mutation is a G>C mutation.

In some embodiments, the template comprises a mismatch to the genomic sequence, optionally 1-4 mismatches to the genomic sequence. In some embodiments, the mismatch that directs a template-based edit in the genomic DNA is within nucleotides T1-T25, optionally within nucleotides T1-T15 of the template. In some embodiments, the template-based edit in the genomic DNA comprises a substitution. In some embodiments, the template-based edit in the genomic DNA comprises an insertion of a nucleotide in the genomic sequence. In some embodiments, the template-based edit in the genomic DNA comprises a deletion of a nucleotide in the genomic sequence. In some embodiments, the template-based edit in the genomic sequence results in a change in an amino acid sequence encoded by a locus in which the genomic sequence is present. In some embodiments, the template-based edit in the genomic sequence does not result in a change in an amino acid sequence encoded by a locus in which the genomic sequence is present.

In some embodiments, the template comprises DNA nucleotides. In some embodiments, the 3′ terminal nucleotide of the template is a DNA nucleotide. In some embodiments, the template consists of DNA nucleotides from nucleotide 4 (T4) from the 3′ end of the template to the 5′ end of the template. In some embodiments, the template consists of DNA nucleotides. In some embodiments, the template comprises RNA nucleotides. In some embodiments, the template comprises 0-1 RNA nucleotides, optionally 1 RNA nucleotide, at the 3′ end of the template. In some embodiments, the template comprises 1-3 RNA nucleotides, optionally only 1 RNA nucleotide, at the 3′ end of the template.

### DNA-Dependent DNA Polymerase Recruiting Sequence

In some embodiments, the present disclosure provides for a 3′ extension comprising a DNA-dependent DNA polymerase recruiting sequence (DRS). The DRS is complementary to a second region of a non-target strand of a target nucleic acid and forms a duplex with the 3′ end of the flap 5′ of the nick in the genomic DNA (see, e.g., FIGS. 2A and B). When the DRS is base-paired to the 3′ end of the flap 5′ of the nick, this flap-DRS duplex can serve as a primer for synthesis of a new DNA strand that is complementary to the template sequence 5′ of the DRS.

It is to be understood that the DRS is partially or fully complementary to the spacer of the SpyCas9 guide RNA of the tgRNA. On account of this complementary sequence, in certain embodiments, the DRS may form a duplex with the spacer region. One of ordinary skill in the art would appreciate that RNA sequences within DNA-RNA duplexes, e.g., a spacer hybridized to a DRS comprising or consisting of DNA nucleotides, are susceptible to cleavage by RNAse H. Thus, there can be a need for both DRS and spacer sequences that minimize the likelihood of RNAse H cleavage following hybridization of the DRS to the spacer. The present disclosure therefore provides for DRS sequences that lead to enhanced tgRNA stability when paired with a corresponding spacer sequence. Such DRS sequences can reduce or minimize the hybridization of the DRS to the spacer sequence, and/or reduce or minimize RNAse H-mediated cleavage of the tgRNA, should such hybridization occur.

In certain embodiments, the DRS comprises DNA nucleotides. In certain embodiments, the plurality of nucleotides in the DRS are DNA nucleotides. In certain embodiments, the DRS comprises both DNA and RNA nucleotides. In certain embodiments, the DRS comprises an RNA nucleotide at its 3′ end and consecutive DNA nucleotides at its 5′ end. In certain embodiments, the DRS consists of DNA nucleotides. For example, FIG. 2B shows an exemplary DRS comprising DNA nucleotides at every position within the DRS. In such embodiments, the DRS or the spacer may further comprise chemical modifications that reduce the susceptibility of the resulting DRS-spacer duplex to cleavage by RNAse H.

In certain embodiments, the DRS comprises RNA nucleotides. In some embodiments, the DRS comprises modified RNA nucleotides. In some embodiments, the plurality of nucleotides in the DRS are RNA nucleotides. In some embodiments, the DRS of the tgRNA comprises both DNA nucleotides and RNA nucleotides. In some embodiments, the DRS comprises consecutive RNA nucleotides at its 3′ end and consecutive DNA nucleotides at its 5′ end. For example, FIG. 2A shows an exemplary DRS comprising a single DNA nucleotide at its 5′ end and consecutive RNA nucleotides from the penultimate 5′ terminal nucleotide of the DRS (D2) through the end of the DRS. In some embodiments, the DRS consists of RNA nucleotides. In embodiments in which the plurality of nucleotides in the DRS are RNA nucleotides or in which the DRS consists of RNA nucleotides, hybridization between the DRS and the spacer results in an RNA-RNA duplex, which is not susceptible to cleavage by RNAse H.

In some embodiments, 1-7 nucleotides (e.g., 1-7, 1-6, 1-5, 1-4, 1-3, 1-2 nucleotides) at the 5′ end of the DRS (nucleotides D1-D7) are DNA nucleotides. In some embodiments, 1-7 nucleotides at the 5′ end of the DRS (nucleotides D1-D7) are DNA nucleotides, with the remaining nucleotides in the DRS being RNA nucleotides.

In some embodiments, the 3′ nucleotide of the DRS is an RNA nucleotide and the remaining nucleotides in the DRS are DNA nucleotides.

In some embodiments, 1-5 nucleotides (e.g., 1-5, 1-4, 1-3, 1-2 nucleotides) at the 5′ end of the DRS (nucleotides D1-D5) are DNA nucleotides. In some embodiments, 1-5 nucleotides at the 5′ end of the DRS (nucleotides D1-D5) are DNA nucleotides, with the remaining nucleotides in the DRS being RNA nucleotides.

In some embodiments, 1-3 nucleotides (e.g., 1-3, 1-2 nucleotides) at the 5′ end of the DRS (nucleotides D1-D3) are DNA nucleotides. In some embodiments, 1-3 nucleotides at the 5′ end of the DRS (nucleotides D1-D3) are DNA nucleotides, with the remaining nucleotides in the DRS being RNA nucleotides.

In some embodiments, at least one nucleotide at positions D1-D5 of the DRS is a DNA nucleotide. In some embodiments, at least two nucleotides at positions D1-D5 of the DRS are DNA nucleotides. In some embodiments, at least three nucleotides at positions D1-D5 of the DRS are DNA nucleotides. In some embodiments, at least four nucleotides at positions D1-D5 of the DRS are DNA nucleotides. In some embodiments, one nucleotide at positions D1-D5 of the DRS is a DNA nucleotide. In some embodiments, two nucleotides at positions D1-D5 of the DRS are DNA nucleotides. In some embodiments, three nucleotides at positions D1-D5 of the DRS are DNA nucleotides. In some embodiments, four nucleotides at positions D1-D5 of the DRS are DNA nucleotides. In some embodiments, all five nucleotides at positions D1-D5 of the DRS are DNA nucleotides.

In some embodiments, the nucleotide at the 5′ end of the DRS (nucleotide D1) is a DNA nucleotide. In some embodiments, the nucleotide at the 5′ end of the DRS (nucleotide D1) is a DNA nucleotide, with the remaining nucleotides in the DRS being RNA nucleotides. In some embodiments, the penultimate 5′ terminal nucleotide of the DRS (nucleotide D2) is an RNA nucleotide. In some embodiments, positions D1 and D3 of the DRS are DNA nucleotides. In some embodiments, positions D1 and D3 of the DRS are DNA nucleotides, with the remaining nucleotides in the DRS being RNA nucleotides. In some embodiments, positions D1, D3, and D5 of the DRS are DNA nucleotides. In some embodiments, positions D1, D3, and D5 of the DRS are DNA nucleotides, with the remaining nucleotides in the DRS being RNA nucleotides.

In some embodiments, the 5′ terminal and penultimate 5′ terminal nucleotides of the DRS are DNA nucleotides. In some embodiments, 1-7 consecutive 5′ terminal nucleotides of the DRS are DNA nucleotides. In some embodiments, 1, 2, 3, 4, 5, 6, or 7 consecutive 5′ terminal nucleotides of the DRS are DNA nucleotides. In some embodiments, at least 3 consecutive 5′ terminal nucleotides of the DRS are DNA nucleotides. In some embodiments, 3 consecutive 5′ terminal nucleotides of the DRS are DNA nucleotides.

In some embodiments, at least one nucleotide in the 3′ region of the DRS (e.g., a nucleotide at any one of positions one, two, three, four, five, six, or all seven of D6-D13) is a DNA nucleotide. In some embodiments, position D6, D7, D8, D9, D10, D11, D12, or D13 of the DRS is a DNA nucleotide. In some embodiments, at least one, at least two, at least three, at least four, at least five, at least six, at least seven, or at all eight nucleotides at positions D6-D13 of the DRS is a DNA nucleotide. In some embodiments, one, two, three, four, five, six, seven, or all eight nucleotides at positions D6-D13 of the DRS is a DNA nucleotide. In some embodiments, one to three nucleotides at positions D6-D13 of the DRS is a DNA nucleotide. In some embodiments, position D9 of the DRS is a DNA nucleotide. In some embodiments, one or both of positions D8 and D11 of the DRS are DNA nucleotides. In some embodiments, one, two, or three of positions D7, D9, and D11 of the DRS are DNA nucleotides. In some embodiments, one, two, or three of positions D6, D9 and D12 of the DRS are DNA nucleotides.

In some embodiments, the DRS consists of DNA nucleotides. In some embodiments, the spacer may comprise one or more modifications that reduce its susceptibility to degradation by RNAse H following binding to the DRS. For example, spacer nucleotides whose 2′-hydroxyl group does not interact with Cas9 may be modified with a 2′-O-methyl (2′-O-me) or a 2′-fluoro (2′-F) modification. In some embodiments, the spacer of the guide RNA (or tgRNA) comprises a modified nucleotide. In some embodiments, the spacer of the guide RNA comprises a phosphorothioate (PS) modification. In some embodiments, the spacer of the guide RNA comprises a modified nucleotide selected from a 2′-O-methyl (2′-O-Me) modified nucleotide, or a 2′-fluoro (2′-F) modified nucleotide. In some embodiments, the spacer of the guide RNA comprises a 2′-O-methyl (2′-O-Me) modified nucleotide. In some embodiments, the spacer of the guide RNA comprises a 2′-fluoro (2′-F) modified nucleotide. In some embodiments, the spacer of the guide RNA comprises DNA nucleotides. In some embodiments, the spacer of the guide RNA comprises a DNA nucleotide at at least 2, 3, 4, 5, 6, 7, 8, 9, or 10 nucleotides. In some embodiments, the spacer of the guide RNA comprises a DNA nucleotide at 1, 2, 3, 4, 5, 6, 7, 8, 9, 10, or 11 nucleotides. In some embodiments, the spacer of the guide RNA comprises a DNA nucleotide at one or more of positions S11, S12, and S18, optionally at each of positions S11, S12, and S18. In some embodiments, the spacer of the guide RNA comprises a DNA nucleotide at each of position S11. In some embodiments, the spacer of the guide RNA comprises a DNA nucleotide at position S12. In some embodiments, the spacer of the guide RNA comprises a DNA nucleotide at position S18. In some embodiments, the spacer of the guide RNA comprises a DNA nucleotide at each of positions S11 and S12. In some embodiments, the spacer of the guide RNA comprises a DNA nucleotide at each of positions S11 and S18. In some embodiments, the spacer of the guide RNA comprises a DNA nucleotide at each of positions S12 and S18. In some embodiments, the spacer of the guide RNA comprises a DNA nucleotide at each of positions S11, S12, and S18. In some embodiments, the spacer of the guide RNA does not comprise DNA nucleotides.

In some embodiments, the spacer of the guide RNA comprises a modified nucleotide at one or more of the first five nucleotides at the 5′ end, e.g., 1, 2, 3, 4, or 5 of the first five nucleotides at the 5′ end. In some embodiments, the spacer of the guide RNA comprises a modified nucleotide at one or more of the first three nucleotides at the 5′ end of the spacer, e.g., at 1, 2, or 3 of the first three nucleotides at the 5′ end of the spacer. In some embodiments, the spacer of the guide RNA does not comprise a modification at one or more of positions S6, S7, S8, S15, S16, and S19. In some embodiments, the spacer of the guide RNA does not comprise a modification at one or more of positions S5, S6, S7, S8, S11, S12, S15, S16, S18, and S19. In some embodiments, the spacer of the guide RNA comprises a modification (e.g., a 2′Ome or 2′F modification) at one or more of positions S4, S5, S9, S10, S11, S12, S13, S14, S17, S18, and S20. In some embodiments, the spacer of the guide RNA comprises 3 PS linkages between the 4 consecutive 5′ terminal nucleotides of the spacer. In some embodiments, the spacer of the guide RNA comprises a PS linkage between positions S7 and S8. In some embodiments, the spacer of the guide RNA comprises a 2′-O-Me modified nucleotide or a 2′-F modified nucleotide in at least 2, 3, 4, 5, 6, 7, 8, 9, or 10 nucleotides. In some embodiments, the spacer of the guide RNA comprises 2′-O-Me modified nucleotides at each of the first three nucleotides (S1, S2, and S3) at the 5′ end of the spacer. In some embodiments, the spacer of the guide RNA comprises 2′-F modified nucleotides at one or more of positions S9, S10, S11, S13, S14, S17, and S18. In some embodiments, the spacer of the guide RNA comprises a 2′-O-Me modified nucleotide at position S4 and 2′-F modified nucleotides at each of positions S9, S10, S11, S13, S14, S17, and S18. In some embodiments, the spacer of the guide RNA comprises a 2′-O-Me modified nucleotide at each of positions S1, S2, S3, and S4, and 2′-F modified nucleotides at each of positions S9, S10, S11, S13, S14, S17, and S18. In some embodiments, the spacer of the guide RNA comprises a 2′-O-Me modified nucleotide at position S4, 2′-F modified nucleotides at each of positions S9, S10, S11, S13, S14, S17, and S18, and a PS linkage between positions S7 and S8. In some embodiments, the spacer of the guide RNA comprises a 2′-O-Me modified nucleotide at each of positions S1, S2, S3, and S4; 2′-F modified nucleotides at each of positions S9, S10, S11, S13, S14, S17; and S18, and a PS linkage between positions S7 and S8. In some embodiments, the spacer of the guide RNA comprises 2′-O-Me modified nucleotides at one or more of positions S4, S5, S9, S10, S11, S12, S13, S14, S17, S18, and S20. In some embodiments, the spacer of the guide RNA comprises 2′-O-Me modified nucleotides at one or more of positions S1, S2, S3, S4, S5, S9, S10, S11, S12, S13, S14, S17, S18, and S20. In some embodiments, the spacer of the guide RNA comprises 2′-O-Me modified nucleotides at one or more of positions S4, S5, S9, S10, S13, S14, S17 and S20. In some embodiments, the spacer of the guide RNA comprises 2′-O-Me modified nucleotides at each of positions S9, S10, S13, S14, S17, and S20. In some embodiments, the spacer of the guide RNA comprises 2′-O-Me modified nucleotides at each of positions S9, S10, S17, and S20 and DNA nucleotides at each of positions S11, S12, and S18. In some embodiments, the spacer of the guide RNA comprises 2′-O-Me modified nucleotides at each of S1, S2, S3, S9, S10, S13, S14, S17, and S20; and the spacer of the guide RNA does not comprise a modification at each of S4, S5, S6, S7, S8, S11, S12, S15, S16, S18, and S19. In some embodiments, the spacer of the guide RNA comprises 2′-O-Me modified nucleotides at each of S1, S2, S3, S9, S10, S13, S14, S17, and S20; the spacer of the guide RNA comprises DNA nucleotides at each of positions S11, S12, and S18; and the spacer of the guide RNA does not comprise a modification at each of S4, S5, S6, S7, S8, S15, S16, and S19.

In some embodiments, where the DRS consists of DNA nucleotides. In some embodiments, the 3′ terminal nucleotide in the DRS is an RNA nucleotide and the remaining nucleotides in the DRS are DNA nucleotides. Accordingly, the spacer may comprise one or more modifications that reduce its susceptibility to degradation by RNAse H following binding to the DRS. In some embodiments, the spacer of the guide RNA comprises 2′-O-Me modified nucleotides at each of S1, S2, S3, S9, S10, S13, S14, S17, and S20, and unmodified nucleotides at each of S6, S7, S8, S15, S16, and S19, optionally unmodified nucleotides at one, two, three, four, or all of S4, S5, S11, S12, and S18. In some embodiments, the spacer of the guide RNA comprises 2′-O-Me modified nucleotides at each of S1, S2, S3, S9, S10, S13, S14, S17, and S20, DNA nucleotides at each of S11, S12, and S18, and unmodified nucleotides at each of S6, S7, S8, S15, S16, and S19, optionally unmodified nucleotides at one or both of S4 and S5. In some embodiments, the spacer of the guide RNA comprises the sequence of SEQ ID NO: 295 or 296, wherein the N is any nucleotide.

In some embodiments, the DRS consists of DNA nucleotides and (1) the spacer of the guide RNA comprises 2′-O-Me modified nucleotides at each of S1, S2, S3, S9, S10, S13, S14, S17, and S20, and unmodified nucleotides at each of S6, S7, S8, S15, S16, and S19, optionally unmodified nucleotides at one, two, three, four, or all of S4, S5, S11, S12, and S18; or (2) the spacer of the guide RNA comprises 2′-O-Me modified nucleotides at each of S1, S2, S3, S9, S10, S13, S14, S17, and S20, DNA nucleotides at each of S11, S12, and S18, and unmodified nucleotides at each of S6, S7, S8, S15, S16, and S19, optionally unmodified nucleotides at one or both of S4 and S5. In some embodiments, the spacer of the guide RNA comprises the sequence of SEQ ID NO: 295 or 296 wherein the N is any nucleotide.

In some embodiments, the 3′ terminal nucleotide in the DRS is an RNA nucleotide and the remaining nucleotides in the DRS are DNA nucleotides; and (1) the spacer of the guide RNA comprises 2′-O-Me modified nucleotides at each of S1, S2, S3, S9, S10, S13, S14, S17, and S20, and unmodified nucleotides at each of S6, S7, S8, S15, S16, and S19, optionally unmodified nucleotides at one, two, three, four, or all of S4, S5, S11, S12, and S18; or (2) the spacer of the guide RNA comprises 2′-O-Me modified nucleotides at each of S1, S2, S3, S9, S10, S13, S14, S17, and S20, DNA nucleotides at each of S11, S12, and S18, and unmodified nucleotides at each of S6, S7, S8, S15, S16, and S19, optionally unmodified nucleotides at one or both of S4 and S5. In some embodiments, the spacer of the guide RNA comprises the sequence of SEQ ID NO: 295 or 296 wherein the N is any nucleotide.

In some embodiments, the DRS consists of RNA nucleotides. In some embodiments, the DRS consisting of RNA nucleotides comprises of modified RNA nucleotides.

In some embodiments, the DRS comprises a modified nucleotide. In some embodiments, the modified nucleotide is selected from the group: 2′-O-Me modified nucleotide; a 2′-F modified nucleotide; an amine modified nucleotide; an alkoxy modified nucleotide; an LNA modified nucleotide; a methoxyethyl modified nucleotide; a halo modified nucleotide; or a PS linkage modified nucleotide. In some embodiments, the modified nucleotide is 2′-O-Me or a PS linkage modified nucleotide. In some embodiments, 1-3 nucleotides at the 3′ end of the DRS are modified nucleotides. In some embodiments, 1, 2, or 3 nucleotides at the 3′ end of the DRS are modified nucleotides. In some embodiments, the 3′ end of the DRS comprises 1-3 phosphorothioate (PS) linkages, optionally wherein at least one PS linkage is between the last two nucleotides at the 3′ terminus. In some embodiments, the 3′ end of the DRS comprises 1, 2, or 3 phosphorothioate (PS) linkages, optionally wherein at least one PS linkage is between the last two nucleotides at the 3′ terminus. In some embodiments, wherein the 3′ end of the DRS is at least 9 nucleotides in length, the 3′ end of the DRS comprises 2 phosphorothioate linkages between the last three nucleotides at the 3′ terminus.

In some embodiments, the modification in the DRS comprises a phosphorothioate modification. In some embodiments, wherein the modification in the DRS comprises a phosphorothioate modification, the DRS comprises a phosphorothioate modification at or 3′ of position D10 in the DRS sequence. In some embodiments, the DRS comprises PS linkages between 2, 3, 4, 5, or 6 consecutive 3′ terminal nucleotides of the DRS. In some embodiments, the DRS comprises a PS linkage between the 3′ terminal and penultimate 3′ terminal nucleotides of the DRS. In some embodiments, the DRS comprises 3 PS linkages between the 4 consecutive nucleotides at the 3′ end of the DRS. In some embodiments, the DRS comprises a PS linkage between 2 nucleotides other than the 3′ terminal and penultimate 3′ terminal nucleotides of the DRS. In some embodiments, wherein the modification in the DRS comprises a phosphorothioate modification, the DRS does not comprise a phosphorothioate modification at position D1-D9 in the DRS sequence.

In some embodiments, the modification in the DRS comprises a 2′-O-Me modification. In some embodiments, wherein the modification in the DRS comprises a 2′-O-Me modification, the DRS comprises a 2′-O-Me modification at or 3′ of position D4 in the DRS sequence. In some embodiments, wherein the modification in the DRS comprises a 2′-O-Me modification, the DRS does not comprise a 2′-O-Me modification at position D1-D3 in the DRS sequence. In some embodiments, the DRS comprises a 2′-O-Me modification at a nucleotide other than the 5′ terminal and 3′ terminal nucleotides of the DRS. In some embodiments, the 3′ terminal nucleotide of the DRS comprises a 2′-O-Me modification.

In some embodiments, the DRS is at least 6 nucleotides in length. In some embodiments, the DRS is about 6-20 nucleotides in length, e.g., 6-17, 8-17, 10-12, 10-14, 10-16, or 12-14 nucleotides in length. In some embodiments, the DRS is 6-17 nucleotides in length. In some embodiments, the DRS is 10-16 nucleotides in length. In some embodiments, the DRS is 6, 7, 8, 9, 10, 11, 12, 13, 14, 15, 16, 17, 18, 19, or 20 nucleotides in length.

In some embodiments, the DRS comprises a 3′ tail. In some embodiments, the 3′ tail comprises a 1-5 nucleotide tail. In some embodiments, the 3′ tail comprises a 1, 2, or 3 nucleotide tail. In some embodiments, the 3′ tail comprises a terminal uridine nucleotide. In some embodiments, the 3′ tail consists of uridine nucleotides. In some embodiments, the 3′ tail comprises a modified nucleotide, e.g., a modified uridine nucleotide.

In some embodiments, the DRS has 0, 1, 2, 3, or 4 mismatches to the corresponding genomic sequence. In some embodiments, the DRS comprises 0 mismatches to the corresponding genomic sequence. In some embodiments, the DRS comprises 1~4 mismatches to the corresponding genomic sequence. In some embodiments, the first 50% of nucleotides at the 5′ end of the DRS have 0, 1, 2, or 3 mismatches to the corresponding genomic sequence. In some embodiments, the first 50% of nucleotides at the 5′ end of the DRS have 0 mismatches to the corresponding genomic sequence. In some embodiments, the first 50% of nucleotides at the 5′ end of the DRS have 1, 2, or 3 mismatches to the corresponding genomic sequence. In some embodiments, the first four nucleotides at the 5′ end of the DRS have 0, 1, 2, or 3 mismatches to the corresponding genomic sequence. In some embodiments, the first four nucleotides at the 5′ end of the DRS have 0 mismatches to the corresponding genomic sequence. In some embodiments, the first four nucleotides at the 5′ end of the DRS have 1 or 2 mismatches to the corresponding genomic sequence. In some embodiments, the first three nucleotides at the 5′ end of the DRS have 0 mismatches to the corresponding genomic sequence. In some embodiments, the first three nucleotides at the 5′ end of the DRS have 1 mismatch to the corresponding genomic sequence. In some embodiments, the first 6 nucleotides at the 5′ end of the DRS have 0, 1, or 2 mismatches to the corresponding genomic sequence. In some embodiments, the first 10 nucleotides at the 5′ end of the DRS have 0, 1, or 2 mismatches to the corresponding genomic sequence. In some embodiments, 70%-100% of the nucleotides in the first 10 nucleotides at the 5′ end of the DRS are complementary to the corresponding genomic sequence. In some embodiments, the DRS comprises, e.g., further comprises, one or more mismatches in the 3′ half of the DRS.

In some embodiments, the sequence of the DRS that is complementary to the DRS-complementary region of the non-target strand of the DNA duplex target nucleic acid comprises nucleotides D2-D9 of the DRS, optionally D2-D8 or D3-D9 of the DRS.

In some embodiments, when the DRS is less than 100% complementary to the corresponding genomic sequence, the positions that are less than 100% complementary to the corresponding genomic sequence do not introduce changes into a genomic sequence after template-based editing. In some embodiments, a mismatch between the DRS and the genomic sequence is at position D1, D2, or D3, optionally at position D1 or D2, can introduce a template-based edit into the genomic sequence. However, the template-based edit may be less efficient than a template-based edit directed by the template. In certain embodiments, the protein sequence after template-based genome editing may not result in an amino acid change, i.e., silent mutations. That is, due to the degeneracy of the genetic code, most amino acids are specified by more than one codon so that a change in the nucleotide sequence does not necessarily give rise to a change in the sequence that is encoded. It is understood that reducing the complementarity of the DRS to the corresponding genomic sequence similarly reduces the complementarity of the DRS to the spacer sequence of the guide RNA.

In some embodiments, the DRS is 6-18 nucleotides in length, optionally 8-18 or 10-12 nucleotides in length. In some embodiments, the DRS is 10-16 or 12-14 nucleotides in length. In some embodiments, the sequence of the DRS that is complementary to the DRS-complementary region of the non-target strand of the DNA duplex target nucleic acid comprises nucleotides 5-10 5′ of the PAM in the non-target strand of the DNA duplex target nucleic acid. In some embodiments, the DRS comprises a sequence that is complementary to at least 6 nucleotides of the DRS-complementary region of the non-target strand of the DNA duplex target nucleic acid comprising nucleotides D2-D9 of the DRS, optionally D2-D8 or D3-D9 of the DRS.

In some embodiments, the DRS comprises a sequence that is complementary to a DRS-complementary region of a non-target strand of the DNA duplex target nucleic acid comprising nucleotides D6-D13 of the DRS, optionally wherein 1, 2, 3, 4, 5, 6, 7, or 8 of nucleotides D6-D13 are DNA nucleotides. In some embodiments, the DRS comprises a sequence that is not fully complementary to the DRS-complementary region of the non-target strand of the DNA duplex target nuclei acid. In some embodiments, the DRS comprises RNA nucleotides. In some embodiments, the penultimate 5′ terminal nucleotide of the DRS (D2) is an RNA nucleotide. In some embodiments, each nucleotide from the penultimate 5′ nucleotide (D2) to the 3′ end nucleotide in the DRS is an RNA nucleotide. In some embodiments, the 3′ terminal nucleotide of the DRS is an RNA nucleotide. In some embodiments, the DRS consists of RNA nucleotides. In some embodiments, the DRS comprises DNA nucleotides. In some embodiments, the 5′ terminal nucleotide of the DRS (D1) is a DNA nucleotide. In some embodiments, the DRS comprises a 3′ end and a 5′ end, wherein 0-7 nucleotides at the 5′ end of the DRS (nucleotides D1-D7) are DNA nucleotides. In some embodiments, one or more of nucleotides 1-7 nucleotides at the 5′ end of the DRS (nucleotides D1-D7) are DNA nucleotides. In some embodiments, one or more of nucleotides 1-5 at the 5′ end of the DRS (nucleotides D1-D5) are DNA nucleotides, optionally wherein the remaining nucleotides in the DRS are RNA nucleotides. In some embodiments, one or more of nucleotides 1-3 at the 5′ end of the DRS (nucleotides D1-D3), optionally the 5′ terminal nucleotide in the DRS (nucleotide D1), is a DNA nucleotide. In some embodiments, nucleotides 1 and 3 at the 5′ end of the DRS (nucleotides D1 and D3) are DNA nucleotides, optionally wherein the remaining nucleotides in the DRS are RNA nucleotides. In some embodiments, nucleotides 1, 3, and 5 at the 5′ end of the DRS (nucleotides D1, D3, and D5) are DNA nucleotides, optionally wherein the remaining nucleotides in the DRS are RNA nucleotides. In some embodiments, the 3′ terminal nucleotide of the template and the 5′ terminal nucleotide of the DRS are DNA nucleotides, and the penultimate 5′ terminal nucleotide of the DRS is an RNA nucleotide. In some embodiments, the 3′ terminal nucleotide in the DRS is an RNA nucleotide and the remaining nucleotides in the DRS are DNA nucleotides.

In some embodiments, the DRS consists of DNA nucleotides. In some embodiments, the spacer of the guide RNA comprises 2′-O-Me modified nucleotides at one or more of positions S1, S2, S3, S4, S5, S9, S10, S11, S12, S13, S14, S17, S18, and S20. In some embodiments, the spacer of the guide RNA does not comprise a modification at one or more of positions S6, S7, S8, S15, S16, and S19. In some embodiments, (a) the spacer of the guide RNA comprises 2′-O-Me modified nucleotides at each of S1, S2, S3, S9, S10, S13, S14, S17, and S20; and (b) the spacer of the guide RNA does not comprise a modification at each of S4, S5, S6, S7, S8, S11, S12, S15, S16, S18, and S19. In some embodiments, (a) the spacer of the guide RNA comprises 2′-O-Me modified nucleotides at each of S1, S2, S3, S9, S10, S13, S14, S17, and S20; (b) the spacer of the guide RNA comprises DNA nucleotides at each of positions S11, S12, and S18; and (c) the spacer of the guide RNA does not comprise a modification at each of S4, S5, S6, S7, S8, S15, S16, and S19.

In some embodiments, the DRS comprises a 3′ tail at the 3′ end of the DRS, wherein the tail is 1-5 nucleotides, optionally 1-3 nucleotides in length. In some embodiments, the 3′ tail of the DRS comprises an RNA nucleotide. In some embodiments, the 3′ tail comprises a uridine nucleotide, optionally wherein the 3′ terminal nucleotide is a modified uridine nucleotide. In some embodiments, the DRS comprises a modified nucleotide. In some embodiments, the DRS comprises a modified nucleotide selected from: (a) 2′-O-methyl (2′-O-Me) modified nucleotide; (b) 2′-fluoro (2′-F) modified nucleotide; (c) an amine modified nucleotide; (d) an alkoxy modified nucleotide; (e) an LNA modified nucleotide; (f) methoxyethyl modified nucleotide; (g) a halo modified nucleotide; or (h) phosphorothioate (PS) linkage modified nucleotide; optionally, wherein the modified nucleotide is a 2′-O-methyl (2′-O-Me) modified nucleotide or a phosphorothioate (PS) linkage modified nucleotide. In some embodiments, the modification in the DRS comprises a phosphorothioate modification, and the DRS does not comprise a phosphorothioate modification at position D1-D9 in the DRS sequence. In some embodiments, the modification in the DRS comprises a phosphorothioate modification, and the DRS comprises a phosphorothioate modification at or 3′ of position D10 in the DRS sequence. In some embodiments, the modification in the DRS comprises a 2′-O-Me modification, and the DRS does not comprise a 2′-O-Me modification at position D1-D3 in the DRS sequence. In some embodiments, the modification in the DRS comprises a 2′-O-Me modification, and the DRS does not comprise a 2′-O-Me modification at the 5′ terminal and 3′ terminal nucleotides of the DRS. In some embodiments, the modification in the DRS comprises a 2′-O-Me modification, and the DRS comprises a 2′-O-Me modification at or 3′ of position D4 in the DRS sequence. In some embodiments, the modification in the DRS comprises a 2′-O-Me or a 2′-F modification, and the DRS comprises a 2′-O-Me or 2′-F modification at the 3′ terminal nucleotide in the DRS. In some embodiments, the DRS comprises PS linkages between 2, 3, 4, 5, or 6 consecutive 3′ terminal nucleotides of the DRS. In some embodiments, the DRS comprises a PS linkage between the 3′ terminal and penultimate 3′ terminal nucleotides of the DRS. In some embodiments, the DRS comprises 3 PS linkages between the 4 consecutive 3′ terminal nucleotides of the DRS. In some embodiments, the DRS comprises a PS linkage between 2 nucleotides other than the 3′ terminal and penultimate 3′ terminal nucleotides of the DRS. In some embodiments, the DRS comprises a phosphorothioate modification, and wherein the DRS does not comprise a phosphorothioate modification at position D1-D9 in the DRS sequence. In some embodiments, the DRS comprises a phosphorothioate modification at or 3′ of position D10 in the DRS sequence.

### Affinity Tag

In some embodiments, the template guide RNA further comprises an affinity tag, wherein the first polynucleotide or the second polynucleotide are bound to a peptide or a peptide complex comprising a SpyCas9 nickase and a DNA-dependent DNA polymerase by the affinity tag. In some embodiments, the affinity tag mediates binding of the 3′ extension to a corresponding 3′ extension-recruiting domain on a DNA-dependent DNA polymerase or SpyCas9 nickase. In some embodiments, the affinity tag recruits a DNA-dependent DNA polymerase to an RNA: DNA duplex comprising the DRS and a flap with an available 3′ end, 5′ of a nick generated as a result of a single-strand break (i.e., a nick) in the DNA, thereby allowing the DNA-dependent DNA polymerase to initiate DNA synthesis from the 3′ end of the flap with an available 3′ end, 5′ of the nick, with the template sequence that is 5′ of the DRS serving as a template sequence for DNA synthesis.

In some embodiments, an affinity tag is located on the second polynucleotide. In some embodiments, the affinity tag is located 5′ of the template sequence. In some embodiments, the affinity tag is located 3′ of the DRS. In some embodiments, the template guide RNA further comprises two affinity tags. In some embodiments, the first and second affinity tags are the same. In some embodiments, the first and second affinity tags are different.

In some embodiments, the tgRNA comprises one affinity tag located 5′ of the template and has a a 5′ terminus, wherein the affinity tag is an aptamer comprising one or more modified nucleotide at the 5′ end thereof.

In some embodiments, wherein the template guide RNA comprises two affinity tags, both affinity tags are located 5′ of the template sequence. In some embodiments, wherein the template guide RNA comprises two affinity tags, a first affinity tag is located 5′ of the template sequence and a second affinity tag is located 3′ of the DRS.

In some embodiments, the affinity tag comprises an aptamer. In some embodiments, the aptamer comprises a DNA aptamer. In some embodiments, the aptamer comprises an RNA aptamer. In some embodiments, the aptamer is naturally occurring. In some embodiments, the aptamer is a viral nucleic acid and the 3′ extension-recruiting domain is a viral protein. In some embodiments, the aptamer comprises an MS2 aptamer sequence, a PP7 aptamer sequence, or a sequence from a guide RNA that does not bind to a SpyCas9 nickase. In some embodiments, the aptamer comprises an MS2 aptamer sequence (alternatively referred to herein as a MS2-loop, MS2 aptamer, or MS2 RNA loop). In some embodiments, the aptamer comprises an MS2 aptamer sequence comprising a nucleotide sequence that has at least 90%, 93%, 95%, or 100% identity to SEQ ID NO: 601. In some embodiments, the aptamer comprises a PP7 aptamer sequence. In some embodiments, the aptamer comprises a sequence from a guide RNA that does not bind to a SpyCas9 nickase. In some embodiments, the aptamer is not naturally occurring, i.e., synthetic.

In some embodiments, the affinity tag is an aptamer comprising one or more modified nucleotides. In some embodiments, the aptamer sequence is modified. In some embodiments, wherein the aptamer sequence comprises a modified nucleotide selected from 2′-O-methyl (2′-O-Me), 2′-fluoro (2′-F), a phosphorothioate (PS) linkage, an amine, an abasic nucleotide, a dideoxynucleotide, or a non-nucleotide linker such as a PEG. In some embodiments, wherein the aptamer sequence is modified, the modification comprises a 2′-O-Me modified nucleotide or a phosphorothioate PS linkage. In some embodiments, the modified nucleotide comprises a 2′-O-Me modified nucleotide. In some embodiments, the modified nucleotide comprises a PS linkage. It is understood that the tolerance for inclusion of modified nucleotides in an aptamer will be aptamer dependent.

In some embodiments, the aptamer comprises a stem-loop. In some embodiments, wherein the aptamer comprises a stem-loop, the aptamer comprises 2′-O-Me modified nucleotides at each nucleotide in the stem of the stem-loop. In some embodiments, wherein the aptamer comprises a stem-loop, the aptamer comprises 2′-O-Me modified nucleotides at each nucleotide.

In some embodiments, the aptamer is located 5′ of the template sequence. In some embodiments, wherein the aptamer is located 5′ of the template sequence and has a 5′ terminus, one or more modified nucleotides is at the 5′ end of the aptamer. In some embodiments, wherein the aptamer is located 5′ of the template sequence, the aptamer comprises a 5′ end modification. In some embodiments, the 5′ end modification comprises a 2′-O-Me modified nucleotide or PS linkage. In some embodiments, wherein the aptamer is located 5′ of the template sequence, the aptamer comprises a PS linkage between each of the first three nucleotides at the 5′ end. In some embodiments, wherein the aptamer is located 5′ of the template sequence, the aptamer comprises 2′-O-Me modified nucleotides at each of the first two nucleotides at the 5′ end. In some embodiments, wherein the aptamer is located 5′ of the template sequence, the aptamer comprises 2′-O-Me modified nucleotides at each of the first two nucleotides at the 5′ end and a PS linkage between each of the first three nucleotides at the 5′ end. In some embodiments, wherein the aptamer is located 5′ of the template sequence and comprises a stem-loop, the aptamer comprises 2′-O-Me modified nucleotides at each nucleotide in the stem of the stem-loop and a PS linkage between each of the first three nucleotides at the 5′ end.

In some embodiments, the aptamer is located 3′ of the DRS. In some embodiments, wherein the aptamer is located 3′ of the DRS, the aptamer comprises a 3′ end modification. In some embodiments, the 3′ end modification comprises a 2′-O-Me modified nucleotide or PS linkage. In some embodiments, wherein the aptamer is located 3′ of the DRS, the aptamer comprises a PS linkage between the last two nucleotides at the 3′ end. In some embodiments, wherein the aptamer is located 3′ of the DRS and comprises a stem-loop, the aptamer comprises 2′-O-Me modified nucleotides at each nucleotide in the stem of the stem-loop and a PS linkage between the last two nucleotides at the 3′ end. In some embodiments, the aptamer has the modification pattern of SEQ ID NO: 601.

In some embodiments, the template guide RNA comprises two aptamers. In some embodiments, the first aptamer is located 5′ of the template sequence and the second aptamer is located 3′ of the DRS. If the aptamer has a free 5′ end, the aptamer may include 5′ end modifications. If the aptamer has a free 3′ end, the aptamer may include 3′ end modifications. In some embodiments, the first or second aptamer has the modification pattern of SEQ ID NO: 601. In some embodiments, the first and second aptamers are the same. In some embodiments, the first and second aptamers are different.

In some embodiments, the aptamer, the template sequence, and the DRS are linked in one of the following orientations: (a) 5′-aptamer-template sequence-DRS-3′; (b) 5′-template sequence-DRS-aptamer-3′; or (c) 5′-aptamer-template sequence-DRS-aptamer-3′. In some embodiments, the template guide RNA comprises one aptamer and the aptamer, the template sequence, and the DRS are linked in the following orientation: 5′-aptamer-template sequence-DRS-3′. In some embodiments, the template guide RNA comprises one aptamer and the aptamer, the template sequence, and the DRS are linked in the following orientation: 5′-template sequence-DRS-aptamer-3′. In some embodiments, the template guide RNA comprises two aptamers and the aptamer, the template sequence, and the DRS are linked in the following orientation: 5′-aptamer-template sequence-DRS-aptamer-3′. In some embodiments, the aptamer is linked to the template sequence or the DRS by an internal linker. In some embodiments, a 3′ extension-recruiting domain (i.e., an aptamer binding domain) for binding the aptamer is present on the DNA-dependent DNA polymerase.

In some embodiments, the affinity tag comprises a small molecule. In some embodiments, wherein the affinity tag comprises a small molecule, the affinity tag comprises biotin, desthiobiotin, or digoxigenin. In some embodiments, the affinity tag comprises biotin. In some embodiments, the affinity tag comprises desthiobiotin. In some embodiments, the affinity tag comprises digoxigenin. In some embodiments, wherein the affinity tag comprises a small molecule, a small molecule-binding domain for binding the small molecule is present on a DNA-dependent DNA polymerase.

In some embodiments, wherein the tgRNA comprises an affinity tag, the 5′ terminal and penultimate 5′ terminal nucleotides of the DRS are DNA nucleotides. In some embodiments, 1-7 consecutive 5′ terminal nucleotides of the DRS are DNA nucleotides. In some embodiments, 1, 2, 3, 4, 5, 6, or 7 consecutive 5′ terminal nucleotides of the DRS are DNA nucleotides. In some embodiments, at least 3 consecutive 5′ terminal nucleotides of the DRS are DNA nucleotides. In some embodiments, 3 consecutive 5′ terminal nucleotides of the DRS are DNA nucleotides.

In some embodiments, wherein the tgRNA comprises an affinity tag, the DRS comprises a modified nucleotide. In some embodiments, the DRS comprises a modified nucleotide selected from a 2′-O-methyl(2′-O-Me) modified nucleotide, a 2′-fluoro (2′-F) modified nucleotide, an amine modified nucleotide, an alkoxy modified nucleotide, an LNA modified nucleotide, a methoxyethyl modified nucleotide, a halo modified nucleotide, or a phosphorothioate (PS) linkage modified nucleotide, optionally wherein the modified nucleotide is a 2′-O-methyl (2′-O-Me) modified nucleotide or a phosphorothioate (PS) linkage modified nucleotide. In some embodiments, the modification in the DRS comprises a phosphorothioate modification. In some embodiments, wherein the modification in the DRS comprises a phosphorothioate modification, the DRS comprises a phosphorothioate modification at or 3′ of position D10 in the DRS sequence. In some embodiments, the modification in the template comprises a phosphorothioate modification. In some embodiments, wherein the modification in the DRS comprises a 2′-O-Me modification, the DRS comprises a 2′-O-Me modification at or 3′ of position D4 in the DRS sequence. In some embodiments, wherein the modification in the DRS comprises a 2′-O-Me modification, the DRS does not comprise a 2′-O-Me modification at position D1-D3 in the DRS sequence.

In some embodiments, the tgRNA further comprises an affinity tag, wherein the first polynucleotide or the second polynucleotide are bound to a peptide or a peptide complex comprising an nCas9 and a DNA-dependent DNA polymerase by the affinity tag. In some embodiments, 1-7 consecutive 5′ terminal nucleotides of the DRS are DNA nucleotides. In some embodiments, the 5′ terminal and penultimate 5′ terminal nucleotides of the DRS are DNA nucleotides. In some embodiments, at least 3 consecutive 5′ terminal nucleotides of the DRS are DNA nucleotides. In some embodiments, 3 consecutive 5′ terminal nucleotides of the DRS are DNA nucleotides. In some embodiments, the affinity tag is located 5′ of the template sequence or the affinity tag is located 3′ of the DRS. In some embodiments, the tgRNA comprises two affinity tags. In some embodiments, the tgRNA comprises two affinity tags, further wherein both affinity tags are located 5′ of the template. In some embodiments, the tgRNA comprises two affinity tags, further wherein a first affinity tag is located 5′ of the template sequence and a second affinity tag is located 3′ of the DRS, wherein the DNA-dependent DNA polymerase is T5 DNA polymerase. In some embodiments, the affinity tag is an aptamer comprising one or more modified nucleotides. In some embodiments, the tgRNA comprises one affinity tag located 5′ of the template and has a 5′ terminus, and wherein the affinity tag is an aptamer comprising one or more modified nucleotide at the 5′ end thereof. In some embodiments, the modified nucleotide is a 2′-O-methyl (2′-O-Me) modified nucleotide or a phosphorothioate (PS) linkage modified nucleotide. In some embodiments, the aptamer is located 5′ of the template sequence, further wherein the aptamer comprises a PS linkage between each of the first three nucleotides at the 5′ end. In some embodiments, the aptamer is located 3′ of the DRS, further wherein the affinity tag comprises a PS linkage between the last two nucleotides at the 3′ end. In some embodiments, the aptamer is located 5′ of the template sequence, further wherein the aptamer comprises 2′-O-Me modified nucleotides at each of the first two nucleotides at the 5′ end. In some embodiments, the aptamer comprises a stem-loop, further wherein the aptamer comprises 2′-O-Me modified nucleotides at each nucleotide in the stem of the stem-loop. In some embodiments, the aptamer comprises 2′-O-Me modified nucleotides at each nucleotide. In some embodiments, the aptamer is located 5′ of the template sequence, further wherein the aptamer comprises 2′-O-Me modified nucleotides at each of the first two nucleotides at the 5′ end and a PS linkage between each of the first three nucleotides at the 5′ end. In some embodiments, the aptamer is located 5′ of the template sequence and comprises a stem-loop, further wherein the aptamer comprises 2′-O-Me modified nucleotides at each nucleotide in the stem of the stem-loop and a PS linkage between each of the first three nucleotides at the 5′ end. In some embodiments, the aptamer is located 3′ of the DRS and comprises a stem-loop, further wherein the aptamer comprises 2′-O-Me modified nucleotides at each nucleotide in the stem of the stem-loop and a PS linkage between the last two nucleotides at the 3′ end.

### Exemplary Template Guide RNAs

In some embodiments, the present disclosure provides for exemplary tgRNAs comprising a first polynucleotide and a second polynucleotide described herein.

Provided herein is also a template guide RNA (tgRNA), wherein the tgRNA comprises a first polynucleotide and a second polynucleotide, wherein: (a) the first polynucleotide comprises a SpyCas9 guide RNA comprising, from 5′ to 3′: (i) a spacer that is complementary to a target region of a target strand of a DNA duplex target nucleic acid; and (ii) a scaffold; and the second polynucleotide is operably linked to the first polynucleotide and comprises, from 5′ to 3′: (i) (i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; and (ii) a DNA-dependent DNA polymerase recruiting sequence (DRS) having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid, optionally wherein the DRS is 6-20 nucleotides in length, further optionally 10-14 nucleotides in length. In some embodiments, the DRS comprises at least one of: (a) a sequence not fully complementary to the DRS-complementary region of the non-target strand of the DNA duplex target nucleic acid; (b) a 1-5 nucleotide, optionally a 1-3 nucleotide 3′ terminal tail comprising a 3′ terminal modified uridine nucleotide; or a terminal (D1) DNA nucleotide and a penultimate 5′ terminal (D2) RNA nucleotide wherein the 3′ end of the first polynucleotide is covalently linked to the 5′ end of the second polynucleotide by a phosphorothioate or phosphodiester linkage.

In some embodiments, the first polynucleotide is covalently linked to the second polynucleotide. In some embodiments, the 3′ end of the first polynucleotide is covalently linked to the 5′ end of the second polynucleotide. In some embodiments, the first polynucleotide is linked to the second polynucleotide by a non-nucleotide linker. In some embodiments, the first polynucleotide is linked to the second polynucleotide by a phosphodiester bond or a phosphorothioate bond.

In some embodiments, the first polynucleotide is not covalently linked to the second polynucleotide. In some embodiments, 1-7 consecutive 5′ terminal nucleotides of the DRS are DNA nucleotides. In some embodiments, the 5′ terminal and penultimate 5′ terminal nucleotides of the DRS are DNA nucleotides. In some embodiments, at least 3 consecutive 5′ terminal nucleotides of the DRS are DNA nucleotides. In some embodiments, 3 consecutive 5′ terminal nucleotides of the DRS are DNA nucleotides

In some embodiments, the present disclosure provides for a tgRNA comprising a first polynucleotide and a second polynucleotide, wherein (1) the first polynucleotide comprises a guide RNA (gRNA) comprising: (a) a spacer sequence of 17-20 nucleotides in length, and (b) a conserved portion comprising the nucleotide sequence of SEQ ID NO: 27 or 40, wherein (i) nucleotides 5′-GUUUUAGAGCUAGAAAUAGCAAGUUAAAAU-3′ or 5′-GUUUUAGACGUAGAAAUACGAAGUUAAAAU-3′ constitute a repeat/antirepeat (R/AR) region, wherein nucleotides 1-6 (LS1-LS6) and 25-30 (LS7-LS12) of the R/AR constitute a lower stem (LS) region, nucleotides 7-8 (B1-B2) and 21-24 (B3-B6) of the R/AR constitute a bulge region; and nucleotides 9-20 (US1-US12) of the R/AR constitute an upper stem (US) region; (ii) nucleotides 5′-AAGGCUAGUCCGUUAUCA-3′ constitute a nexus region (N1-N18); (iii) nucleotides 5′-CGAAAG-3′ constitute a hairpin 1 (H1) region (from 5′ to 3′, H1-2, H1-5 to H1-8, and H1-11); (iv) nucleotides 5′-GCACCGAGUCGGUGC-3′ constitute a hairpin 2 (H2) region H2-1 to H2-15); and (v) a G nucleotide between H1 region and H2 region constitutes nucleotide n; (c) wherein the conserved portion comprises at least one region with unmodified and modified nucleotides selected from: (i) the lower stem region comprising modified nucleotides at LS1, LS8, LS10, and LS12; nucleotides not modified with 2′-O-Me at LS2, LS3, LS4, LS5; (ii) the bulge region comprising nucleotides not modified with 2′-O-Me at B5 and B6; (iii) the upper stem region comprising modified nucleotides at at least 10 positions selected from US1, US2, US3, US4, US5, US6, US7, US8, US9, US10, US11, and US12, optionally at all positions US1, US2, US3, US4, US5, US6, US7, US8, US9, US10, US11, and US12; (iv) the nexus region comprising modified nucleotides at N1, N2, N4, N7, N11, N12, and N17; and nucleotides not modified with 2′-O-Me at N6, N8, N9, N10, N13, and N14; (v) the hairpin 1 (H1) region, comprising modified nucleotides at H1-2, H1-6, H1-7, H1-8, and H1-11; and nucleotides not modified with 2′-O-Me at H1-5; (vi) the hairpin 2 (H2) region comprising modified nucleotides at H2-1, H2-2, H2-3, H2-4, H2-5, H2-6, H2-7, H2-12, H2-13, H2-14, and H2-15; and nucleotides not modified with 2′-O-Me at H2-8, H2-9, and H2-10; and (vii) the n is a modified or unmodified; (d) wherein the gRNA comprises a 5′ end modification; and (2) the second polynucleotide comprises (a) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; and (b) a DNA-dependent DNA polymerase recruiting sequence (DRS) that is 6-20 nucleotides in length, optionally 10-14 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid. In some embodiments, the DRS of the tgRNA comprises RNA nucleotides. In some embodiments, the DRS of the tgRNA comprises DNA nucleotides. In some embodiments, the DRS of the tgRNA comprises RNA nucleotide(s) and DNA nucleotide(s). In some embodiments, the DRS comprises at least one of: (a) a sequence not fully complementary to the DRS-complementary region of the non-target strand of the DNA duplex target nucleic acid; (b) a 1-5 nucleotide, optionally a 1-3 nucleotide 3′ terminal tail comprising a 3′ terminal modified uridine nucleotide; or (c) a 5′ terminal (D1) DNA nucleotide and a penultimate 5′ terminal (D2) RNA nucleotide wherein the 3′ end of the first polynucleotide is covalently linked to the 5′ end of the second polynucleotide by a phosphorothioate or phosphodiester linkage. In some embodiments, the template is about 15 nucleotides in length, e.g., 15 nucleotides in length. In some embodiments, the DRS is about 12 nucleotides in length, e.g., 12 nucleotides in length. In some embodiments, the tgRNA comprises one or more internal linkers. In some embodiments, one or both of the R/AR and hairpin 1 region comprises an internal linker. In some embodiments, the R/AR region comprises an internal linker. In some embodiments, the internal linker substitutes for about 8 nucleotides within the R/AR region, e.g., 8 nucleotides within the R/AR region. In some embodiments, the internal linker substitutes for US3 through US10. In some embodiments, the hairpin 1 region comprises an internal linker. In some embodiments, internal linker substitutes for about 6 nucleotides within the hairpin 1 region, e.g., 6 nucleotides within the hairpin 1 region. In some embodiments, the internal linker substitutes for H1-5 through H1-8. In some embodiments, the internal linker is L1. In some embodiments, the tgRNA does not comprise an internal linker. In some embodiments, the first polynucleotide comprises a sequence selected from any one of SEQ ID NOs: 310, 325, 347-349, 434, and 438, or a sequence that is at least 85%, 90%, or 95% identical to any one of SEQ ID NOs: 310, 325, 347-349, 434, and 438. In some embodiments, the first polynucleotide comprises a sequence selected from SEQ ID NOs: 347 and 434.

In some embodiments, the present disclosure provides for a tgRNA comprising a first polynucleotide and a second polynucleotide, wherein (1) the first polynucleotide comprises a guide RNA (gRNA) comprising: (a) a spacer sequence of 17-20 nucleotides in length, and (b) a conserved portion comprising the nucleotide sequence of SEQ ID NO: 27 or 40, wherein (i) nucleotides 5′-GUUUUAGAGCUAGAAAUAGCAAGUUAAAAU-3′ or 5′-GUUUUAGACGUAGAAAUACGAAGUUAAAAU-3′ constitute a repeat/antirepeat (R/AR) region, wherein nucleotides 1-6 (LS1-LS6) and 25-30 (LS7-LS12) of the R/AR constitute a lower stem (LS) region, nucleotides 7-8 (B1-B2) and 21-24 (B3-B6) of the R/AR constitute a bulge region; and nucleotides 9-20 (US1-US12) of the R/AR constitute an upper stem (US) region; (ii) nucleotides 5′-AAGGCUAGUCCGUUAUCA-3′ constitute a nexus region (N1-N18); (iii) nucleotides 5′-CGAAAG-3′ constitute a hairpin 1 (H1) region (from 5′ to 3′, H1-2, H1-5 to H1-8, and H1-11); (iv) nucleotides 5′-GCACCGAGUCGGUGC-3′ constitute a hairpin 2 (H2) region H2-1 to H2-15); and (v) a G nucleotide between H1 region and H2 region constitutes nucleotide n; (c) wherein the conserved portion comprises at least one region with unmodified and modified nucleotides selected from: (i) the lower stem region comprising modified nucleotides at LS1, LS8, LS10, and LS12; nucleotides not modified with 2′-O-Me at LS2, LS3, LS4, LS5; (ii) the bulge region comprising nucleotides not modified with 2′-O-Me at B5 and B6; (iii) the upper stem region comprising modified nucleotides at at least 10 positions selected from US1, US2, US3, US4, US5, US6, US7, US8, US9, US10, US11, and US12, optionally at all positions US1, US2, US3, US4, US5, US6, US7, US8, US9, US10, US11, and US12; (iv) the nexus region comprising modified nucleotides at N1, N2, N4, N7, N11, N12, and N17; and nucleotides not modified with 2′-O-Me at N6, N8, N9, N10, N13, and N14; (v) the hairpin 1 (H1) region, comprising modified nucleotides at H1-2, H1-6, H1-7, H1-8, and H1-11; and nucleotides not modified with 2′-O-Me at H1-5; (vi) the hairpin 2 (H2) region comprising modified nucleotides at H2-1, H2-2, H2-3, H2-4, H2-5, H2-6, H2-7, H2-12, H2-13, H2-14, and H2-15; and nucleotides not modified with 2′-O-Me at H2-8, H2-9, and H2-10; and (vii) the n is a modified or unmodified; (d) wherein the gRNA comprises a 5′ end modification; and (2) the second polynucleotide comprises (a) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; (b) a DNA-dependent DNA polymerase recruiting sequence (DRS) that is 6-18 nucleotides in length, optionally 8-18 or 10-12 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid. In some embodiments, the DRS of the tgRNA comprises RNA nucleotides. In some embodiments, the DRS of the tgRNA comprises DNA nucleotides. In some embodiments, the DRS of the tgRNA comprises RNA nucleotide(s) and DNA nucleotide(s).

In some embodiments, the template is about 15 nucleotides in length, e.g., 15 nucleotides in length. In some embodiments, the DRS is about 12 nucleotides in length, e.g., 12 nucleotides in length. In some embodiments, the tgRNA comprises one or more internal linkers. In some embodiments, one or both of the R/AR and hairpin 1 region comprises an internal linker. In some embodiments, the R/AR region comprises an internal linker. In some embodiments, the internal linker substitutes for about 8 nucleotides within the R/AR region, e.g., 8 nucleotides within the R/AR region. In some embodiments, the internal linker substitutes for US3 through US10. In some embodiments, the hairpin 1 region comprises an internal linker. In some embodiments, internal linker substitutes for about 6 nucleotides within the hairpin 1 region, e.g., 6 nucleotides within the hairpin 1 region. In some embodiments, the internal linker substitutes for H1-5 through H1-8. In some embodiments, the internal linker is L1. In some embodiments, the tgRNA does not comprise an internal linker. In some embodiments, the first polynucleotide comprises a sequence selected from any one of SEQ ID NOs: 310, 325, 347-349, 434, and 438, or a sequence that is at least 85%, 90%, or 95% identical to any one of SEQ ID NOs: 310, 325, 347-349, 434, and 438. In some embodiments, the first polynucleotide comprises a sequence selected from SEQ ID NOs: 347 and 434.

In some embodiments, the present disclosure provides for a tgRNA comprising a first polynucleotide and a second polynucleotide, wherein (1) the first polynucleotide comprises a guide RNA (gRNA) comprising: (a) a spacer sequence of 17-20 nucleotides in length, and (b) a conserved portion comprising the nucleotide sequence of SEQ ID NO: 27 or 40, wherein (i) nucleotides 5′-GUUUUAGAGCUAGAAAUAGCAAGUUAAAAU-3′ or 5′-GUUUUAGACGUAGAAAUACGAAGUUAAAAU-3′ constitute a repeat/antirepeat (R/AR) region, wherein nucleotides 1-6 (LS1-LS6) and 25-30 (LS7-LS12) of the R/AR constitute a lower stem (LS) region, nucleotides 7-8 (B1-B2) and 21-24 (B3-B6) of the R/AR constitute a bulge region; and nucleotides 9-20 (US1-US12) of the R/AR constitute an upper stem (US) region; (ii) nucleotides 5′-AAGGCUAGUCCGUUAUCA-3′ constitute a nexus region (N1-N18); (iii) nucleotides 5′-CGAAAG-3′ constitute a hairpin 1 (H1) region (from 5′ to 3′, H1-2, H1-5 to H1-8, and H1-11); (iv) nucleotides 5′-GCACCGAGUCGGUGC-3′ constitute a hairpin 2 (H2) region H2-1 to H2-15); and (v) a G nucleotide between H1 region and H2 region constitutes nucleotide n; (c) wherein the conserved portion comprises at least one region with unmodified and modified nucleotides selected from: (i) the lower stem region comprising modified nucleotides at LS1, LS8, LS10, and LS12; nucleotides not modified with 2′-O-Me at LS2, LS3, LS4, LS5; (ii) the bulge region comprising nucleotides not modified with 2′-O-Me at B5 and B6; (iii) the upper stem region comprising modified nucleotides at at least 10 positions selected from US1, US2, US3, US4, US5, US6, US7, US8, US9, US10, US11, and US12, optionally at all positions US1, US2, US3, US4, US5, US6, US7, US8, US9, US10, US11, and US12; (iv) the nexus region comprising modified nucleotides at N1, N2, N4, N7, N11, N12, and N17; and nucleotides not modified with 2′-O-Me at N6, N8, N9, N10, N13, and N14; (v) the hairpin 1 (H1) region, comprising modified nucleotides at H1-2, H1-6, H1-7, H1-8, and H1-11; and nucleotides not modified with 2′-O-Me at H1-5; (vi) the hairpin 2 (H2) region comprising modified nucleotides at H2-1, H2-2, H2-3, H2-4, H2-5, H2-6, H2-7, H2-12, H2-13, H2-14, and H2-15; and nucleotides not modified with 2′-O-Me at H2-8, H2-9, and H2-10; and (vii) the n is a modified or unmodified; (d) wherein the gRNA comprises a 5′ end modification; and (2) the second polynucleotide comprises (a) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; (b) a DNA-dependent DNA polymerase recruiting sequence (DRS) that is 6-20 nucleotides in length, optionally 10-16 or 12-14 nucleotides in length, and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid comprising nucleotides 5-10 5′ of the PAM in the non-target strand of the DNA duplex target nucleic acid. In some embodiments, the DRS of the tgRNA comprises RNA nucleotides. In some embodiments, the DRS of the tgRNA comprises DNA nucleotides. In some embodiments, the DRS of the tgRNA comprises RNA nucleotide(s) and DNA nucleotide(s).

In some embodiments, the template is about 15 nucleotides in length, e.g., 15 nucleotides in length. In some embodiments, the DRS is about 12 nucleotides in length, e.g., 12 nucleotides in length. In some embodiments, the tgRNA comprises one or more internal linkers. In some embodiments, one or both of the R/AR and hairpin 1 region comprises an internal linker. In some embodiments, the R/AR region comprises an internal linker. In some embodiments, the internal linker substitutes for about 8 nucleotides within the R/AR region, e.g., 8 nucleotides within the R/AR region. In some embodiments, the internal linker substitutes for US3 through US10. In some embodiments, the hairpin 1 region comprises an internal linker. In some embodiments, internal linker substitutes for about 6 nucleotides within the hairpin 1 region, e.g., 6 nucleotides within the hairpin 1 region. In some embodiments, the internal linker substitutes for H1-5 through H1-8. In some embodiments, the internal linker is L1. In some embodiments, the tgRNA does not comprise an internal linker. In some embodiments, the first polynucleotide comprises a sequence selected from any one of SEQ ID NOs: 310, 325, 347-349, 434, and 438, or a sequence that is at least 85%, 90%, or 95% identical to any one of SEQ ID NOs: 310, 325, 347-349, 434, and 438. In some embodiments, the first polynucleotide comprises a sequence selected from SEQ ID NOs: 347 and 434.

Exemplary Methods of Producing tgRNAs

Template guide RNAs (tgRNAs) provided herein may be produced using methods known in the art or otherwise provided herein. In some embodiments, the tgRNAs herein are produced using solid-phase synthesis. Alternatively, in some embodiments, the tgRNAs are produced from two or more separate polynucleotides (e.g., two or three polynucleotides) that are subsequently ligated together to form a ligated tgRNA. The ligation may be mediated by one or more splint oligonucleotides (e.g., one or two splint oligonucleotides). A splint oligonucleotide is an oligonucleotide that is capable of annealing to the ends of two polynucleotides that flank a ligation site, thereby bringing the two polynucleotides into proximity so that they may be ligated together (e.g., via a ligase).

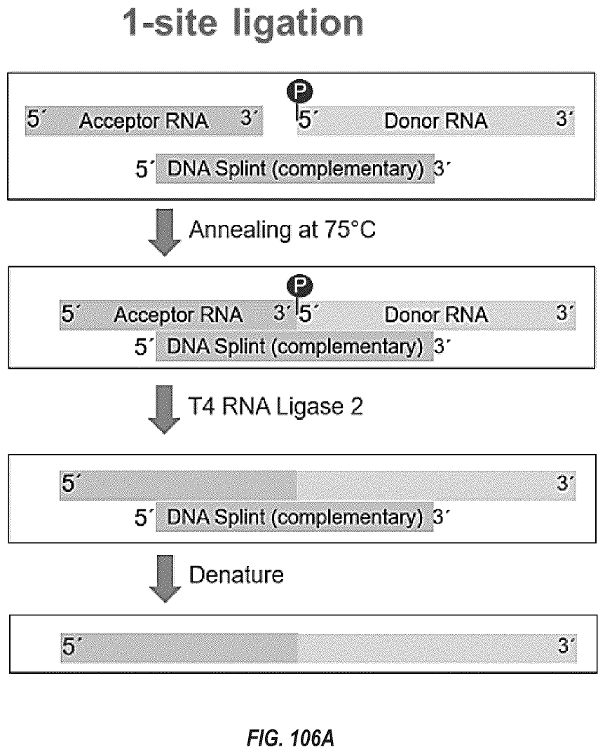

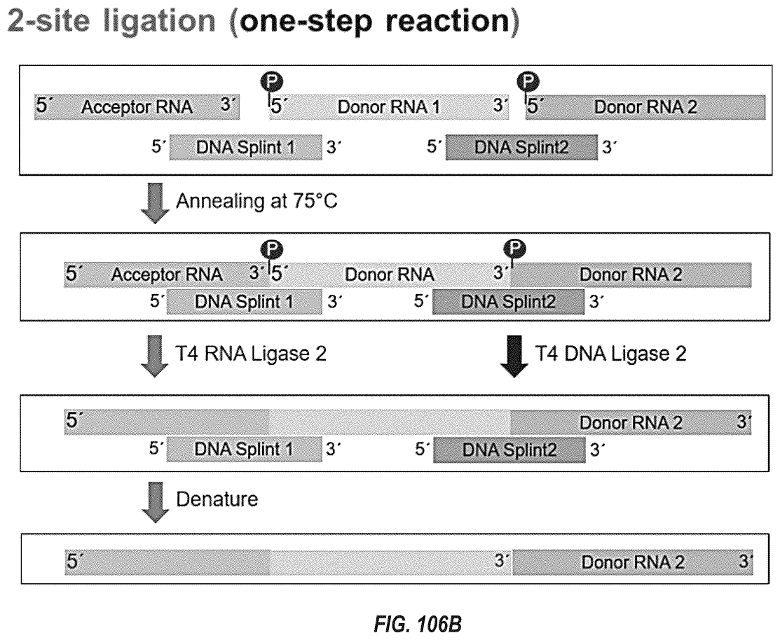

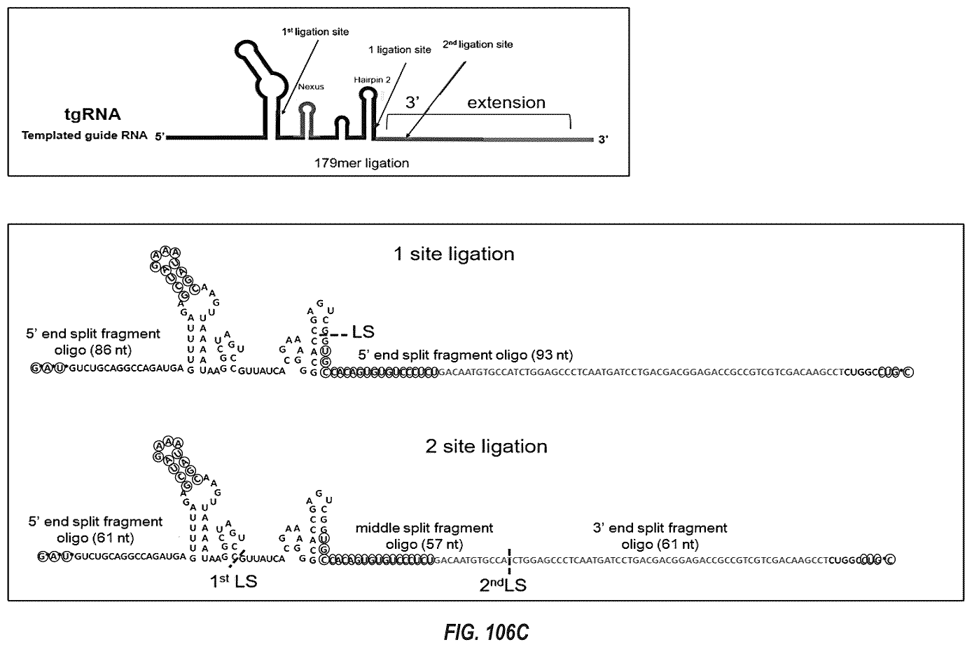

In some embodiments, a ligated tgRNA is produced by ligating together two polynucleotides (i.e., joined at one ligation site, as shown in FIG. 106A). In some such embodiments, the ligation is mediated by one splint oligonucleotide. Accordingly in some embodiments, provided herein is a method of producing a ligated tgRNA the method comprising: providing a first polynucleotide comprising a 3′ end; providing a second polynucleotide comprising a 5′ end; providing a splint oligonucleotide capable of annealing to nucleotides at the 3′ end of the first polynucleotide and nucleotides at the 5′ end of the second polynucleotide; providing a ligase to ligate the 3′ end of the first polynucleotide to the 5′ end of the second polynucleotide thereby forming a ligated tgRNA. In some such embodiments, the 5′ end of the second polynucleotide comprises a 5′ phosphate moiety.

In some embodiments, a ligated tgRNA is produced by ligating together three polynucleotides (i.e., joined at two ligation sites, as shown in FIG. 106B). In some such embodiments, the ligation is mediated by two splint oligonucleotides. Accordingly in some embodiments, provided herein is a method of producing a ligated tgRNA the method comprising: providing a first polynucleotide comprising a 3′ end; providing a second polynucleotide comprising a 3′ and 5′ end; providing a third polynucleotide comprising a 5′ end. providing a first splint oligonucleotide capable of annealing to nucleotides at the 3′ end of the first polynucleotide and nucleotides at the 5′ end of the second polynucleotide; providing a second splint oligonucleotide capable of annealing to nucleotides at the 3′ end of the second polynucleotide and nucleotides at the 5′ end of the third polynucleotide; providing one or more ligases to ligate (i) the 3′ end of the first polynucleotide to the 5′ end of the second polynucleotide and (ii) the 3′ end of the second polynucleotide to the 5′ end of the third polynucleotide thereby forming a ligated tgRNA. In some embodiments, the 5′ ends of both the second polynucleotide and the third polynucleotide comprise a 5′ phosphate moiety.

In some embodiments, the ligated tgRNA comprises a SpyCas9 guide RNA, a template sequence, and a DNA-dependent DNA polymerase recruiting sequence (DRS). In some embodiments, the ligated tgRNA comprises in order from 5′ to 3′ a SpyCas9 guide RNA, a template sequence, and a DNA-dependent DNA polymerase recruiting sequence (DRS).

In some embodiments, the ligated tgRNA is at least 100, 110, 120, 130, 140, 150, 160, 170, 180, 190, or 200 nucleotides in length. In some embodiments, the ligated tgRNA is 100-110, 110-120, 120-130, 130-140, 140-150, 150-160, 160-170, 170-180, 180-190, or 190-200 nucleotides in length. In some embodiments, the ligated tgRNA is 100-110, 100-120, 100-130, 100-140, 100-150, 100-160, 100-170, 100-180, 100-190, or 100-200 nucleotides in length. In some embodiments, the ligated tgRNA is 110-120, 110-130, 110-140, 110-150, 110-160, 110-170, 110-180, 110-190, or 110-200 nucleotides in length. In some embodiments, the ligated tgRNA is 120-130, 120-140, 120-150, 120-160, 120-170, 120-180, 120-190, or 120-200 nucleotides in length. In some embodiments, the ligated tgRNA is 130-140, 130-150, 130-160, 130-170, 130-180, 130-190, or 130-200 nucleotides in length. In some embodiments, the ligated tgRNA is 140-150, 140-160, 140-170, 140-180, 140-190, or 140-200 nucleotides in length. In some embodiments, the ligated tgRNA is 150-160, 150-170, 150-180, 150-190, or 150-200 nucleotides in length. In some embodiments, the ligated tgRNA is 160-170, 160-180, 160-190, or 160-200 nucleotides in length. In some embodiments, the ligated tgRNA is 170-180, 170-190, or 170-200 nucleotides in length. In some embodiments, the ligated tgRNA is 180-190 or 180-200 nucleotides in length. In some embodiments, the ligated tgRNA is 190-200 nucleotides in length. In some embodiments, the ligated tgRNA is 100-150 nucleotides in length. In some embodiments, the ligated tgRNA is 150-200 nucleotides in length. In some embodiments, the ligated tgRNA is 160-190 nucleotides in length. In some embodiments, the ligated tgRNA is 117 nucleotides in length (a “117 mer”). In some embodiments, the ligated tgRNA is 179 nucleotides in length (a “179 mer”).

In embodiments where the tgRNA is produced by ligating together two polynucleotides, in some embodiments, the first polynucleotide and the second polynucleotide are each about 20 to about 100 nucleotides, about 50 to about 100 nucleotides, about 75 to about 100 nucleotides, about 80 to about 95 nucleotides, or about 85 to about 95 nucleotides in length. In some embodiments, the first polynucleotide and the second polynucleotide are each about 50 to 80 nucleotides in length. In some embodiments, the first polynucleotide and the second polynucleotide are 50-55, 55-60, 60-65, 65-70, 70-75, or 75-80 nucleotides in length. In some embodiments, the first polynucleotide and the second polynucleotide are 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length. some embodiments, wherein the tgRNA is produced by ligating together two polynucleotides, the lengths of the first and second polynucleotide are similar. In some embodiments, the first polynucleotide is no more than 30 nucleotides longer or shorter than the second polynucleotide. In some embodiments, the first polynucleotide is less than 5, 10, 15, 20, 25, or 30 nucleotides longer or shorter than the second polynucleotide. In some embodiments, the first polynucleotide is less than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides longer or shorter than the second polynucleotide. In some embodiments, the first polynucleotide is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides longer or shorter than the second polynucleotide. In some embodiments, the first and second polynucleotide are equal in length.

In embodiments where the tgRNA is produced by ligating together three polynucleotides, in some embodiments, the first polynucleotide, the second polynucleotide, and the third polynucleotide are each independently about 10 to about 90 nucleotides, about 20 to about 80 nucleotides, about 25 to about 75 nucleotides, about 50 to about 70 nucleotides, about 50 to about 65 nucleotides in length. In some embodiments, the first polynucleotide, the second polynucleotide, and the third polynucleotide are each about 50 to 80 nucleotides in length. In some embodiments, the first polynucleotide, the second polynucleotide, and the third polynucleotide are 50-55, 55-60, 60-65, 65-70, 70-75, or 75-80 nucleotides in length. In some embodiments, the first polynucleotide, the second polynucleotide, and the third polynucleotide are 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length. In some embodiments, wherein the tgRNA is produced by ligating together three polynucleotides, the lengths of the first, second, and third polynucleotides are similar. In some embodiments, the first polynucleotide is no more than 30 nucleotides longer or shorter than the second or third polynucleotide. In some embodiments, the first polynucleotide is less than 5, 10, 15, 20, 25, or 30 nucleotides longer or shorter than the second or third polynucleotide. In some embodiments, the first polynucleotide is less than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides longer or shorter than the second or third polynucleotide. In some embodiments, the first polynucleotide is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides longer or shorter than the second or third polynucleotide. In some embodiments, the first, second, and third polynucleotide are equal in length.

In some embodiments, the splint oligonucleotide is a DNA oligonucleotide. In some embodiments, the splint oligonucleotide is an RNA oligonucleotide. In some embodiments, the splint oligonucleotide is about 20 to about 100 nucleotides, about 20 to about 90 nucleotides, about 20 to about 80 nucleotides, about 20 to about 70 nucleotides, about 20 to about 60 nucleotides, about 20 to about 50 nucleotides, about 20 to about 40 nucleotides, or about 20 to about 30 nucleotides in length. In some embodiments, the splint oligonucleotide is about 35-50 nucleotides. In some embodiments, the splint oligonucleotide is about 40-50 nucleotides. In some embodiments, the splint oligonucleotide is about 40-60 nucleotides. In some embodiments, the splint oligonucleotide is about 40-70 nucleotides. In some embodiments, the splint oligonucleotide is about 15 to 30 nucleotides in length. In some embodiments, the splint oligonucleotide is about 15 to 25 nucleotides in length, for example, about 15, 16, 17, 18, 19, 20, 21, 22, 23, 24, 25, 26, 27, 28, 29, and 30 nucleotides in length. In some embodiments, the splint oligonucleotide is 15, 16, 17, 18, 19, 20, 21, 22, 23, 24, 25, 26, 27, 28, 29, and 30 nucleotides in length. In some embodiments, the splint oligonucleotide is about 25 to 30 nucleotides in length, In some embodiments, the splint oligonucleotide is about 25 to 30 nucleotides in length, for example, about 25, 26, 27, 28, 29, or 30 nucleotides in length. In some embodiments, the splint oligonucleotide is 25, 26, 27, 28, 29, or 30 nucleotides in length. In one embodiment, the splint oligonucleotide is about 27 nucleotides in length. In one embodiment, the splint oligonucleotide is 27 nucleotides in length. In one embodiment, the splint oligonucleotide is at least about 35 nucleotides in length. In one embodiment, the splint oligonucleotide is at least about 40 nucleotides in length.

In some embodiments, the splint oligonucleotide has complementarity to at least 5, 6, 7, 8, 9, 10, 11, 12, 13, 14, 15, 16, 17, 18, 19, 20, 21, 22, 23, 24, 25, 26, 27, 28, 29, 30, or more of each polynucleotide in the ligation reaction (e.g., the first polynucleotide and/or the second polynucleotide, and optionally a third polynucleotide). In some embodiments, the splint oligonucleotide has complementarity to 5, 6, 7, 8, 9, 10, 11, 12, 13, 14, 15, 16, 17, 18, 19, 20, 25, 30, or more of each polynucleotide in the ligation reaction (e.g., the first polynucleotide and/or the second polynucleotide, and optionally a third polynucleotide).

In some embodiments, the first splint oligonucleotide has complementarity to at least 10 nucleotides at the 3′ end of the first polynucleotide. In some embodiments, the first splint oligonucleotide has complementarity to at least 10 nucleotides at the 5′ end of the second polynucleotide. In some embodiments, the second splint oligonucleotide has complementarity to at least 10 nucleotides at the 3′ end of the second polynucleotide. In some embodiments, the second splint oligonucleotide has complementarity to at least 10 nucleotides at the 5′ end of the third polynucleotide.

In some embodiments, the splint oligonucleotide comprises RNA. In some embodiments, the splint oligonucleotide comprises DNA. In some embodiments, the splint oligonucleotide comprises RNA and DNA. In some embodiments, the first splint oligonucleotide is an RNA splint oligonucleotide. In some embodiments, the first splint oligonucleotide is a DNA splint oligonucleotide. In some embodiments, the first splint oligonucleotide is an RNA splint oligonucleotide and the second splint oligonucleotide is an RNA splint oligonucleotide. In some embodiments, the first splint oligonucleotide is a DNA splint oligonucleotide and the second splint oligonucleotide is a DNA splint oligonucleotide. In some embodiments, the first splint oligonucleotide is an RNA splint oligonucleotide and the second splint oligonucleotide is a DNA splint oligonucleotide. In some embodiments, the first splint oligonucleotide is a DNA splint oligonucleotide and the second splint oligonucleotide is an RNA splint oligonucleotide.

Ligation can be performed using a ligase, such as a DNA ligase or an RNA ligase. In some embodiments, the ligation is performed with a DNA ligase (e.g., where the ligation site is flanked by DNA nucleotides). In some embodiments, the ligation is performed with an RNA ligase (e.g., where the ligation site is flanked by RNA nucleotides). In some embodiments, a ligation involving two ligation sites is performed with both a DNA ligase and an RNA ligase (e.g, where a first ligation site is flanked by DNA nucleotides and a second ligation site is flanked by RNA nucleotides). In some embodiments, the ligation is performed using both a DNA ligase and an RNA ligase in the same reaction mixture. For example, in some embodiments, the ligation is performed using both a DNA ligase and an RNA ligase simultaneously.

A variety of ligases may be used in the presently described methods. For example, the ligase can be a T4 DNA ligase, T4 RNA ligase 1, T4 RNA ligase 2, RtcB ligase, T3 DNA ligase, T7 DNA ligase, Taq DNA ligase, PBCV-1 DNA ligase, thermostable DNA ligase (e.g., 5′AppDNA/RNA ligase), an ATP dependent DNA ligase, or SplintR ligase. In instances where both a DNA ligase and a RNA ligase are simultaneously used, In some embodiments, the DNA ligase is a T4 DNA ligase (e.g., T4 DNA ligase 4) and the RNA ligase is a T4 RNA ligase (e.g., a T4 RNA ligase 2).

In some embodiments, the ligation occurs at a ligation site within a scaffold of the SpyCas9 guide RNA. In some embodiments, the ligation site within the scaffold of the SpyCas9 guide RNA is not within a loop region. In some embodiments, the ligation site within the scaffold is not within a loop region but is within 1, 2, 3, 4, or 5 nucleotides of a loop region. In some embodiments, the ligation site within the scaffold is not within a loop region but is within 4 or 5 nucleotides of a loop region. In some embodiments, the ligation site within the scaffold of the SpyCas9 guide RNA is within a hairpin 2 region. In some embodiments, the ligation occurs at a ligation site within the 3′ extension region of the tgRNA (e.g., the template sequence or DRS).

In some embodiments, wherein the ligated tgRNA is formed from two polynucleotides, the last nucleotide at the 3′ end of the first polynucleotide is not modified. In some embodiments, wherein the ligated tgRNA is formed from two polynucleotides, the first nucleotide at the 5′ end of the second polynucleotide is not modified. In some embodiments, wherein the ligated tgRNA is formed from two polynucleotides, the last nucleotide at the 3′ end of the first polynucleotide and the first nucleotide at the 5′ end of the second polynucleotide are not modified.

In instances where the ligated tgRNA is formed from three polynucleotides, ligation may occur at two sites (e.g., a first ligation site and a second ligation site). In some embodiments, the first ligation occurs at a first ligation site that is flanked by RNA nucleotides and a second ligation occurs at a second ligation site that is flanked by DNA nucleotides. In some embodiments, a first ligation occurs at a first ligation site within a scaffold of the SpyCas9 guide RNA. In some embodiments, the first ligation site within the scaffold of the SpyCas9 guide RNA is within 1-2 nucleotides of a nexus region. In some embodiments, the first ligation site is within 2 nucleotides of a nexus region. In some embodiments, the first ligation site is within 1 nucleotide of a nexus region. In some embodiments, the first ligation site is within a nexus region. In some embodiments, a second ligation occurs at a second ligation site within the 3′ extension region of the tgRNA. In some embodiments, a second ligation occurs at a second ligation site within a template sequence of the tgRNA. In some embodiments, a second ligation occurs at a second ligation site within the DRS of the tgRNA.

In some embodiments, wherein the ligated tgRNA is formed from three polynucleotides, the last nucleotide at the 3′ end of the first polynucleotide is not modified. In some embodiments, wherein the ligated tgRNA is formed from three polynucleotides, the first nucleotide at the 5′ end of the second polynucleotide is not modified. In some embodiments, wherein the ligated tgRNA is formed from three polynucleotides, the last nucleotide at the 3′ end of the second polynucleotide is not modified. In some embodiments, wherein the ligated tgRNA is formed from three polynucleotides, the first nucleotides at the 5′ end of the third polynucleotide is not modified. In some embodiments, wherein the ligated tgRNA is formed from three polynucleotides, the last nucleotide at the 3′ end of the first polynucleotide, the first nucleotide at the 5′ end of the second polynucleotide, the last nucleotide at the 3′ end of the second polynucleotide, and the first nucleotide at the 5′ end of the third polynucleotide are not modified.

To anneal the splint oligonucleotide(s) to the first polynucleotide and/or second polynucleotide (and/or optionally third polynucleotide), in some embodiments, the polynucleotides and the splint oligonucleotide can be denatured, i.e., the intramolecular structures of the polynucleotides and/or the splint oligonucleotide are disrupted to allow for annealing between the polynucleotides and the splint oligonucleotide. Denaturing can be achieved, for example, by heating a solution containing the polynucleotides and the splint oligonucleotide to, to about, or to at least about, 37° C., 38° C., 39° C., 40° C., 42° C., 44° C., 46° C., 48° C., 50° C., 52° C., 54° C., 56° C., 58° C., 60° C., 65° C., 70° C., 75° C., 80° C., 85° C., 90° C., 95° C., 100° C., or a number or a range between any two of these values (e.g., for a set period of time, e.g., 5 minutes).

In some embodiments, the annealing process includes cooling the solution to a temperature of, or of about, 0° C., 2° C., 4° C., 6° C., 8° C., 10° C., 12° C., 14° C., 16° C., 18° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., or a number or a range between any two of these values, after heating. For example, in some embodiments, hybridizing includes cooling the solution to about 4° C. after heating. In some embodiments, hybridizing includes cooling the solution to about 4° C. for 40 minutes. Hybridizing can include cooling the solution to a temperature at which a ligase retains ligase activity sufficient to ligate the first and second polynucleotides (and optionally, third polynucleotide), and/or to a temperature below the melting temperature of the complex formed by the polynucleotides and the splint oligonucleotide upon hybridization.

Ligating the first and second polynucleotides (and optionally, third polynucleotide) can be carried out at various temperatures, for example about 15° C. to about 40° C., and optionally the ligating is carried out at about 15° C., 20° C., 22° C., 24° C., 27° C., 30° C., 33° C., 34° C., 35° C., 36° C., or 37° C. The ligation reaction forming a tgRNA can be carried out for various duration, for example, a duration of, or of about, 10 minutes, 20 minutes, 30 minutes, 40 minutes, 50 minutes, 1 hour, 1.5 hours, 2 hours, 3 hours, 4 hours, 5 hours, 6 hours, 7 hours, 8 hours, 9 hours, 10 hours, 12 hours, 14 hours, 16 hours, 18 hours, 20 hours, 25 hours, 30 hours, 35 hours, 40 hours, 45 hours, or a number or a range between any two of these values. In some embodiments, the ligation reaction is carried out for 30 min to 60 min (e.g., 40 min).

After ligation, in some embodiments, the method further comprises separating, isolating and/or purifying the ligated tgRNA. In some embodiments, the method can include separating the splint oligonucleotides from the ligated tgRNAs after ligation. The separated splint oligonucleotides and/or the ligated tgRNAs can be isolated and/or purified in accordance to any method known in the art such as ultra-filtration and chromatographic methods. In some embodiments, the ligated tgRNA is separated from the splint oligonucleotide(s) by chromatography (e.g., HPLC).

In some embodiments, the ligated tgRNA is separated from impurities such as protein and salts by RNA precipitation. In some embodiments, the ligated tgRNA is separated from the ligase by RNA precipitation. In some embodiments, precipitation is accomplished using an RNA precipitation buffer. In one embodiment, the RNA precipitation comprises 25 mM sodium citrate, 0.2% (v/v)N-lauroylsarcosine sodium, and 46% of guanidine thiocyanate. In some embodiments, the RNA precipitation buffer may be prepared by mixing sodium citrate buffer (pH 6.5), N-lauroylsarcosine sodium, guanidine thiocyanate, and water. In some embodiments, the RNA precipitation buffer may be prepared by mixing 400 mL of 250 mM sodium citrate buffer, pH 6.5, with 26.67 mL of 30% (v/v)N-lauroylsarcosine sodium, 1840 g of guanidine thiocyanate, and 1733 mL of water. In some embodiments, the splint oligonucleotides and ligated tgRNAs are mixed with the RNA precipitation buffer. In some embodiments, the splint oligonucleotides, ligated tgRNAs, and RNA precipitation buffer are further mixed with ethanol. The solution may be chilled, e.g., at −20° C., for an hour and centrifuged, yielding an RNA pellet, which may be further washed in ethanol, air dried, and resuspended in water. In some embodiments, the solution is chilled. In some embodiments, the solution is chilled for an hour. In some embodiments, the solution is chilled for at least an hour. In some embodiments, the solution is centrifuged. In some embodiments, the pellet is further washed at least one, two, or three times. In some embodiments, the pellet is further washed at least one, two, or three times with ethanol. In some embodiments, the pellet is air dried. In some embodiments, the air dried pellet is resuspended in water.

In some embodiments, the ligated tgRNA is subjected to one or more purification steps (e.g., in addition to or alternatively to an initial separation step). In some embodiments, the purification steps comprises subjecting the ligated tgRNA to high-performance liquid chromatography (HPLC). For example, in some embodiments, the purification step comprises subjecting the ligated tgRNA to HPLC on a Waters Acquity Peptide BEH C18 2.5 μM, 4.6×150 mm column. In some embodiments, the column is coupled to an ultraviolet detector.

In some embodiments, wherein the purification step comprises subjecting the ligated tgRNA to HPLC, the mobile phase A comprises triethylamine acetate (TEAA). In some embodiments, wherein the purification step comprises subjecting the ligat In some embodiments, the pellet is further washed at least one, two, or three times with ethanol. In some embodiments, the pellet is washed in about 70 to 95% ethanol, for example, about 70%, 75%, 80%, 85%, 90%, or 95% ethanol. In some embodiments, the pellet is washed in 70%, 75%, 80%, 85%, 90%, or 95% ethanol. In some embodiments, the pellet is air dried. In some embodiments, the air dried pellet is resuspended in water. ed tgRNA to HPLC, the mobile phase A comprises 100 mM triethylamine acetate (TEAA).

In some embodiments, wherein the purification step comprises subjecting the ligated tgRNA to HPLC, the mobile phase B comprises TEAA. In some embodiments, wherein the purification step comprises subjecting the ligated tgRNA to HPLC, the mobile phase B comprises acetonitrile. In some embodiments, wherein the purification step comprises subjecting the ligated tgRNA to HPLC, the mobile phase B comprises TEAA and acetonitrile. In some embodiments, wherein the purification step comprises subjecting the ligated tgRNA to HPLC, the mobile phase B comprises 100 mM TEAA and 40% acetonitrile.

In some embodiments, wherein the purification step comprises subjecting the ligated tgRNA to HPLC, the column is operated at 50° C., 55° C., 60° C., 65° C., 70° C., 75° C., 80° C., 85° C., 90° C., or 95° C. In some embodiments, wherein the purification step comprises subjecting the ligated tgRNA to HPLC, the column is operated at 75° C. In some embodiments, wherein the purification step comprises subjecting the ligated tgRNA to HPLC, the column is operated with a flow rate of 100, 150, 200, 250, 300, 350, 400, 450, 500, 550, 600, 650, 700, 750, 800, 850, or 900 μL/min. In some embodiments, wherein the purification step comprises subjecting the ligated tgRNA to HPLC, the column is operated with a flow rate of 500 μL/min. In some embodiments, wherein the purification step comprises subjecting the ligated tgRNA to HPLC, the column is operated with a gradient comprising a linear increase from 5% to 50%, e.g., 5% to 10%, 5% to 15%, 5% to 20%, 5% to 25%, 5% to 30% 5% to 35%, 5% to 40%, 5% to 45%, 5% to 50%, 10% to 15%, 10% to 20%, 10% to 25%, 10% to 30% 10% to 35%, 10% to 40%, 10% to 45%, 10% to 50%, 15% to 20%, 15% to 25%, 15% to 30% 15% to 35%, 15% to 40%, 15% to 45%, 15% to 50%, 20% to 25%, 20% to 30% 20% to 20%, 20% to 40%, 20% to 45%, 20% to 50%, 25% to 30% 25% to 35%, 25% to 40%, 25% to 45%, 25% to 50%, 30% to 35%, 30% to 40%, 30% to 45%, 30% to 50%, 35% to 40%, 35% to 45%, 35% to 50%, 40% to 45%, 40% to 50%, or 45 to 50% mobile phase B. In some embodiments, wherein the purification step comprises subjecting the ligated tgRNA to HPLC, the column is operated with a gradient comprising a linear increase from 10% to 30% mobile phase B. In some embodiments, the linear increase is over 5, 10, 15, 20, 25, 30, 35, 40, 45, 50, 55, 60, 65, 70, 75, 80, 85, 90, 95, 100, 105, or 110 minutes. In some embodiments, the linear increase is over 55 minutes.

In some embodiments, wherein the purification step comprises subjecting the ligated tgRNA to HPLC, a 1, 2, 3, 4, 5, 6, 7, 8, 9, or 10 minute wash with mobile phase B is performed. In some embodiments, a 5 minute wash with mobile phase B is then performed. In some embodiments, the wash with mobile phase B is followed by a final column equilibration time of 1, 2, 3, 4, 5, 6, 7, 8, 9, 10, 11, 12, 13, 14, 15, 16, 17, 18, 19, or 20 minutes.

In some embodiments, the wash with mobile phase B is followed by a final column equilibration time of 10 minutes. In some embodiments, fraction collection is enabled based on a retention time window.

In some embodiments, the ligated tgRNA is concentrated and buffer-exchanged. In some embodiments, the ligated tgRNA is concentrated and buffer-exchanged to water using a 15 mL-3 kDa cut-of Amicon filter.

In some embodiments, the HPLC is reversed-phase HPLC (RP-HPLC).

In some embodiments, the purification step comprises subjecting the ligated tgRNA to ultra-performance liquid chromatography (UPLC). For example, in some embodiments, the purification step comprises subjecting the ligated tgRNA to UPLC on a Waters Acquity Peptide BEH C18 1.7 μM, 2.1×150 mm column coupled to an ultraviolet detector and a Thermo QExactive high-resolution mass spectrometer (UPLC-UV-HRMS).

In some embodiments, wherein the purification step comprises subjecting the ligated tgRNA to UPLC, the mobile phase A comprises 1,1,1,3,3,3-hexafluoroisopropanol (HFIP). In some embodiments, the mobile phase A comprises hexylamine (HA). In some embodiments, the mobile phase A comprises methanol. In some embodiments, the mobile phase A comprises 1,1,1,3,3,3-hexafluoroisopropanol (HFIP), hexylamine (HA), and methanol. In some embodiments, wherein the purification step comprises subjecting the ligated tgRNA to UPLC, the mobile phase A comprises 150 mM 1,1,1,3,3,3-hexafluoroisopropanol (HFIP), 20 mM hexylamine (HA), and 5% methanol.

In some embodiments, wherein the purification step comprises subjecting the ligated tgRNA to UPLC, the mobile phase B comprises HFIP. In some embodiments, wherein the purification step comprises subjecting the ligated tgRNA to UPLC, the mobile phase B comprises HA. In some embodiments, wherein the purification step comprises subjecting the ligated tgRNA to UPLC, the mobile phase B comprises methanol. In some embodiments, wherein the purification step comprises subjecting the ligated tgRNA to UPLC, the mobile phase B comprises acetonitrile. In some embodiments, wherein the purification step comprises subjecting the ligated tgRNA to UPLC, the mobile phase B comprises HFIP, HA, methanol, and acetonitrile. In some embodiments, wherein the purification step comprises subjecting the ligated tgRNA to UPLC, the mobile phase B comprises 75 mM HFIP, 10 mM HA, and 50% each of methanol and acetonitrile.

In some embodiments, wherein the purification step comprises subjecting the ligated tgRNA to UPLC, the mobile phase C comprises methanol. In some embodiments, wherein the purification step comprises subjecting the ligated tgRNA to UPLC, the mobile phase C comprises formic acid. In some embodiments, wherein the purification step comprises subjecting the ligated tgRNA to UPLC, the mobile phase C comprises methanol and formic acid. In some embodiments, wherein the purification step comprises subjecting the ligated tgRNA to UPLC, the mobile phase C comprises methanol containing 0.2% formic acid.

In some embodiments, wherein the purification step comprises subjecting the ligated tgRNA to UPLC, the column is operated at 50° C., 55° C., 60° C., 65° C., 70° C., 75° C., 80° C., 85° C., or 90° C. In some embodiments, wherein the purification step comprises subjecting the ligated tgRNA to UPLC, the column is operated at 70° C. In some embodiments, the column is operated with a flow rate of 50, 75, 100, 125, 150, 175, 200, 225, 250, 275, 300, 325, or 350 μL/min. In some embodiments, the column is operated with a flow rate of 200 μL/min. In some embodiments, the column is operated with a gradient comprising a first linear increase from 5% to 50%, e.g., 5% to 10%, 5% to 15%, 5% to 20%, 5% to 25%, 5% to 30% 5% to 35%, 5% to 40%, 5% to 45%, 5% to 50%, 10% to 15%, 10% to 20%, 10% to 25%, 10% to 30% 10% to 35%, 10% to 40%, 10% to 45%, 10% to 50%, 15% to 20%, 15% to 25%, 15% to 30% 15% to 35%, 15% to 40%, 15% to 45%, 15% to 50%, 20% to 25%, 20% to 30% 20% to 20%, 20% to 40%, 20% to 45%, 20% to 50%, 25% to 30% 25% to 35%, 25% to 40%, 25% to 45%, 25% to 50%, 30% to 35%, 30% to 40%, 30% to 45%, 30% to 50%, 35% to 40%, 35% to 45%, 35% to 50%, 40% to 45%, 40% to 50%, or 45 to 50% mobile phase B. In some embodiments, the column is operated with a gradient comprising a first linear increase from 10% to 35% mobile phase B. In some embodiments, the linear increase is over 0.5, 1.0, 1.5, 2.0, 2.5, 3.0, 3.5, 4.0, 5.0, 5.5, 6.0, 6.5, 7.0, 7.5, 8.0, 8.5, 9.0, 9.5, or 10.0 minutes. In some embodiments, the linear increase is over 5.5 minutes. In some embodiments, the linear increase occurs in background of mobile phase A. In some embodiments, the mobile phase A is 90%-65%. In some embodiments, the first linear increase is followed by a second linear increase. In some embodiments, the second linear increase is to 55% mobile phase B/45% mobile phase A. In some embodiments, the linear increase is over 20, 22, 24, 26, 28, 30, 32, 34, 36, 38, 40, 42, 44, 46, 48, 50, 52, 54, 56, 58, or 60 minutes. In some embodiments, the linear increase is over 38 minutes.

In some embodiments, wherein the purification step comprises subjecting the ligated tgRNA to UPLC, a 5 minute wash is performed. In some embodiments, the wash is performed with mobile phase B. In some embodiments, the wash is performed with 100% mobile phase B. In some embodiments, the wash with mobile phase B is followed by a 5 minute wash with mobile phase C. In some embodiments, the wash is performed with 100% mobile phase C. In some embodiments, a final column equilibration to mobile phase A and B is performed. In some embodiments, the final column equilibration is to 90% mobile phase B/10% mobile phase A. In some embodiments, the final column equilibration over 10 minutes.

In some embodiments, the method is effective to increase the amount of full length product (FLP) of the ligated tgRNA relative to a the amount prior to the separation, isolation, or purification step. In some embodiments, the purification step results in a ligated tgRNA that is at least 50%, 60%, 70%, 80%, 85%, 90%, 95%, or 98% in purity, or a number or a range between any two of these values.

In some embodiments, the methods disclosed herein produce yields of at least 50 mg, for example, 50-100 mg, 50-1000 mg, or 50-10,000 mg.

In some embodiments, the methods disclosed herein produce yields of 50-100 mg. In some embodiments, the methods disclosed herein produce yields of 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, or 100 mg.

In some embodiments, the methods disclosed herein produce yields of 50-55 mg, 55-60 mg, 60-65 mg, 65-70 mg, 70-75 mg, 75-80 mg, 80-85 mg, 85-90 mg, 90-95 mg, or 95-100 mg. In some embodiments, the methods disclosed herein produce yields of 50-55 mg, 50-60 mg, 50-65 mg, 50-70 mg, 50-75 mg, 50-80 mg, 50-85 mg, 50-90 mg, 50-95 mg, or 50-100 mg. In some embodiments, the methods disclosed herein produce yields of 55-60 mg, 55-65 mg, 55-70 mg, 55-75 mg, 55-80 mg, 55-85 mg, 55-90 mg, 55-95 mg, or 55-100 mg. In some embodiments, the methods disclosed herein produce yields of 60-65 mg, 60-70 mg, 60-75 mg, 60-80 mg, 60-85 mg, 60-90 mg, 60-95 mg, or 60-100 mg. In some embodiments, the methods disclosed herein produce yields of 65-70 mg, 65-75 mg, 65-80 mg, 65-85 mg, 65-90 mg, 65-95 mg, or 65-100 mg. In some embodiments, the methods disclosed herein produce yields of 70-75 mg, 70-80 mg, 70-85 mg, 70-90 mg, 70-95 mg, or 70-100 mg. In some embodiments, the methods disclosed herein produce yields of 75-80 mg, 77-85 mg, 75-90 mg, 75-95 mg, or 75-100 mg. In some embodiments, the methods disclosed herein produce yields of 80-85 mg, 80-90 mg, 80-95 mg, or 80-100 mg. In some embodiments, the methods disclosed herein produce yields of 85-90 mg, 85-95 mg, or 85-100 mg. In some embodiments, the methods disclosed herein produce yields of 90-95 mg, or 90-100 mg. In some embodiments, the methods disclosed herein produce yields of 95-100 mg.

In some embodiments, the methods disclosed herein produce yields of 50-1000 mg. In some embodiments, the methods disclosed herein produce yields of 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, or 1000 mg.

In some embodiments, the methods disclosed herein produce yields of 50-100 mg, 50-150 mg, 50-200 mg, 50-250 mg, 50-300 mg, 50-350 mg, 50-400 mg, 50-450 mg, 50-500 mg, 50-550 mg, 50-600 mg, 50-650 mg, 50-700 mg, 50-750 mg, 50-800 mg, 50-850 mg, 50-900 mg, 50-950 mg, or 50-1000 mg. In some embodiments, the methods disclosed herein produce yields of 100-150 mg, 100-200 mg, 100-250 mg, 100-300 mg, 100-350 mg, 100-400 mg, 100-450 mg, 100-500 mg, 100-550 mg, 100-600 mg, 100-650 mg, 100-700 mg, 100-750 mg, 100-800 mg, 100-850 mg, 100-900 mg, or 100-950 mg, or 100-1000 mg. In some embodiments, the methods disclosed herein produce yields of 150-200 mg, 150-250 mg, 150-300 mg, 150-350 mg, 150-400 mg, 150-450 mg, 150-500 mg, 150-550 mg, 150-600 mg, 150-650 mg, 150-700 mg, 150-750 mg, 150-800 mg, 150-850 mg, 150-900 mg, 150-950 mg, or 150-1000 mg. In some embodiments, the methods disclosed herein produce yields of 200-250 mg, 200-300 mg, 200-350 mg, 200-400 mg, 200-450 mg, 200-500 mg, 200-550 mg, 200-600 mg, 200-650 mg, 200-700 mg, 200-750 mg, 200-800 mg, 200-850 mg, 200-900 mg, 200-950 mg, or 200-1000 mg. In some embodiments, the methods disclosed herein produce yields of 250-300 mg, 250-350 mg, 250-400 mg, 250-450 mg, 250-500 mg, 250-550 mg, 250-600 mg, 250-650 mg, 250-700 mg, 250-750 mg, 250-800 mg, 250-850 mg, 250-900 mg, 250-950 mg, or 250-1000 mg. In some embodiments, the methods disclosed herein produce yields of 300-350 mg, 300-400 mg, 300-450 mg, 300-500 mg, 300-550 mg, 300-600 mg, 300-650 mg, 300-700 mg, 300-750 mg, 300-800 mg, 300-850 mg, 300-900 mg, 300-950 mg, or 300-1000 mg. In some embodiments, the methods disclosed herein produce yields of 350-400 mg, 350-450 mg, 350-500 mg, 350-550 mg, 350-600 mg, 350-650 mg, 350-700 mg, 350-750 mg, 350-800 mg, 350-850 mg, 350-900 mg, 350-950 mg, or 350-1000 mg. In some embodiments, the methods disclosed herein produce yields of 400-450 mg, 400-500 mg, 400-550 mg, 400-600 mg, 400-650 mg, 400-700 mg, 400-750 mg, 400-800 mg, 400-850 mg, 400-900 mg, 400-950 mg, or 400-1000 mg. In some embodiments, the methods disclosed herein produce yields of 450-500 mg, 450-550 mg, 450-600 mg, 450-650 mg, 450-700 mg, 450-750 mg, 450-800 mg, 450-850 mg, 450-900 mg, 450-950 mg, or 450-1000 mg. In some embodiments, the methods disclosed herein produce yields of 500-550 mg, 500-600 mg, 500-650 mg, 500-700 mg, 500-750 mg, 500-800 mg, 500-850 mg, 500-900 mg, 500-950 mg, or 500-1000 mg. In some embodiments, the methods disclosed herein produce yields of 550-600 mg, 550-650 mg, 550-700 mg, 550-750 mg, 550-800 mg, 550-850 mg, 550-900 mg, 550-950 mg, or 550-1000 mg. In some embodiments, the methods disclosed herein produce yields of 600-650 mg, 600-700 mg, 600-750 mg, 600-800 mg, 600-850 mg, 600-900 mg, 600-950 mg, or 600-1000 mg. In some embodiments, the methods disclosed herein produce yields of 650-700 mg, 650-750 mg, 650-800 mg, 650-850 mg, 650-900 mg, 650-950 mg, or 650-1000 mg. In some embodiments, the methods disclosed herein produce yields of 700-750 mg, 700-800 mg, 700-850 mg, 700-900 mg, 700-950 mg, or 700-1000 mg. In some embodiments, the methods disclosed herein produce yields of 750-800 mg, 750-850 mg, 750-900 mg, 750-950 mg, or 750-1000 mg. In some embodiments, the methods disclosed herein produce yields of 800-850 mg, 800-900 mg, 800-950 mg, or 800-1000 mg. In some embodiments, the methods disclosed herein produce yields of 850-900 mg, 850-950 mg, or 850-1000 mg. In some embodiments, the methods disclosed herein produce yields of 900-950 mg, or 900-1000 mg. In some embodiments, the methods disclosed herein produce yields of 950-1000 mg.

In some embodiments, the methods disclosed herein produce yields of 50-10,000 mg. In some embodiments, the methods disclosed herein produce yields of 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 3050 mg, 3100 mg, 3150 mg, 3200 mg, 3250 mg, 3300 mg, 3350 mg, 3400 mg, 3450 mg, 3500 mg, 3550 mg, 3600 mg, 3650 mg, 3700 mg, 3750 mg, 3800 mg, 3850 mg, 3900 mg, 3950 mg, 4000 mg, 4050 mg, 4100 mg, 4150 mg, 4200 mg, 4250 mg, 4300 mg, 4350 mg, 4400 mg, 4450 mg, 4500 mg, 4550 mg, 4600 mg, 4650 mg, 4700 mg, 4750 mg, 4800 mg, 4850 mg, 4900 mg, 4950 mg, 5000 mg, 5050 mg, 5100 mg, 5150 mg, 5200 mg, 5250 mg, 5300 mg, 5350 mg, 5400 mg, 5450 mg, 5500 mg, 5550 mg, 5600 mg, 5650 mg, 5700 mg, 5750 mg, 5800 mg, 5850 mg, 5900 mg, 5950 mg, 6000 mg, 6050 mg, 6100 mg, 6150 mg, 6200 mg, 6250 mg, 6300 mg, 6350 mg, 6400 mg, 6450 mg, 6500 mg, 6550 mg, 6600 mg, 6650 mg, 6700 mg, 6750 mg, 6800 mg, 6850 mg, 6900 mg, 6950 mg, 7000 mg, 7050 mg, 7100 mg, 7150 mg, 7200 mg, 7250 mg, 7300 mg, 7350 mg, 7400 mg, 7450 mg, 7500 mg, 7550 mg, 7600 mg, 7650 mg, 7700 mg, 7750 mg, 7800 mg, 7850 mg, 7900 mg, 7950 mg, 8000 mg, 8050 mg, 8100 mg, 8150 mg, 8200 mg, 8250 mg, 8300 mg, 8350 mg, 8400 mg, 8450 mg, 8500 mg, 8550 mg, 8600 mg, 8650 mg, 8700 mg, 8750 mg, 8800 mg, 8850 mg, 8900 mg, 8950 mg, 9000 mg, 9050 mg, 9100 mg, 9150 mg, 9200 mg, 9250 mg, 9300 mg, 9350 mg, 9400 mg, 9450 mg, 9500 mg, 9550 mg, 9600 mg, 9650 mg, 9700 mg, 9750 mg, 9800 mg, 9850 mg, 9900 mg, 9950 mg, or 10,000 mg.

In some embodiments, the methods disclosed herein produce yields of 50-1000 mg, 1000-2000 mg, 2000-3000 mg, 3000-4000 mg, 4000-5000 mg, 5000-6000 mg, 6000-7000 mg, 7000-8000 mg, 8000-9000 mg, or 9000-10,000 mg. In some embodiments, the methods disclosed herein yield 50-1000 mg, 50-2000 mg, 50-3000 mg, 50-4000 mg, 50-5000 mg, 50-6000 mg, 50-7000 mg, 50-8000 mg, 50-9000 mg, or 50-10,000 mg. In some embodiments, the methods disclosed herein produce yields of 1000-2000 mg, 1000-3000 mg, 1000-4000 mg, 1000-5000 mg, 1000-6000 mg, 1000-7000 mg, 1000-8000 mg, 1000-9000 mg, or 1000-10,000 mg. In some embodiments, the methods disclosed herein produce yields of 2000-3000 mg, 2000-4000 mg, 2000-5000 mg, 2000-6000 mg, 2000-7000 mg, 2000-8000 mg, 2000-9000 mg, or 2000-10,000 mg. In some embodiments, the methods disclosed herein produce yields of 3000-4000 mg, 3000-5000 mg, 3000-6000 mg, 3000-7000 mg, 3000-8000 mg, 3000-9000 mg, or 3000-10,000 mg. In some embodiments, the methods disclosed herein produce yields of 4000-5000 mg, 4000-6000 mg, 4000-7000 mg, 4000-8000 mg, 4000-9000 mg, or 4000-10,000 mg. In some embodiments, the methods disclosed herein produce yields of 5000-6000 mg, 5000-7000 mg, 5000-8000 mg, 5000-9000 mg, or 5000-10,000 mg. In some embodiments, the methods disclosed herein produce yields of 6000-7000 mg, 6000-8000 mg, 6000-9000 mg, or 6000-10,000 mg. In some embodiments, the methods disclosed herein produce yields of 7000-8000 mg, 7000-9000 mg, or 7000-10,000 mg. In some embodiments, the methods disclosed herein produce yields of 8000-9000 mg, or 8000-10,000 mg. In some embodiments, the methods disclosed herein produce yields of 9000-10,000 mg.

### Enzymes

In some embodiments, the compositions, systems, methods, and other aspects set forth herein, make use of a SpyCas9 nickase that is operably linked to a DNA-dependent DNA polymerase. In some embodiments, the SpyCas9 nickase is operably linked to the DNA-dependent DNA polymerase. In some embodiments, the SpyCas9 nickase is operably linked to a DNA-dependent DNA polymerase and one or more 3′ extension-recruiting domains. In some embodiments, the SpyCas9 nickase generates a single-strand break (i.e., a nick) within a second region of a non-target strand of a target genomic sequence, thereby creating a flap with an available 3′ end, 5′ of the nick. Following hybridization of the DRS to the flap with an available 3′ end, 5′ of the nick, the DNA-dependent DNA polymerase uses the 3′ end of the flap as a primer for DNA synthesis, with the template sequence 5′ of the DRS serving as its template.

### SpyCas9

In some embodiments, the scaffold of the SpyCas9 guide RNA binds to a SpyCas9 nickase. In some embodiments, the SpyCas9 nickase comprises an endonucleolytic active site that has been inactivated, e.g., by one or more alterations (e.g., point mutations) in a catalytic domain. See, e.g., U.S. Pat. No. 8,889,356 for discussion of Cas nickases and exemplary catalytic domain alterations.

Wild type S. pyogenes Cas9 has two catalytic domains: RuvC and HNH. The RuvC domain cleaves the non-target DNA strand, and the HINH domain cleaves the target strand of DNA. In some embodiments, the SpyCas9 nickase may comprise an amino acid substitution in the HINH or HNH-like nuclease domain. Exemplary amino acid substitutions in the HINH or HNH-like nuclease domain include D839K, D839Q, D839N, D839A, H840S, H840T, H840Q, H840D, H840R, H840K, H840Y, H840N, N863S, N863T, N863A, N863E, and K866A.

In some embodiments, the SpyCas9 nickase comprises an amino acid sequence that has at least 90%, 91%, 92%, 93%, 94%, 95%, 96%, 97%, 98%, 99%, or 100% identity to any one of SEQ ID NOs: 1014, 972, 974, 976, 982, 984, 986, 988, 990, 992, 994, 996, 998, and 1000. In some embodiments, the SpyCas9 nickase comprises an amino acid sequence of any one of SEQ ID NOs: 1014, 972, 974, 976, 982, 984, 986, 988, 990, 992, 994, 996, 998, and 1000. In some embodiments, the SpyCas9 nickase is encoded by a nucleotide sequence that has at least 90%, 91%, 92%, 93%, 94%, 95%, 96%, 97%, 98%, 99%, or 100% identity to any one of SEQ ID NOs: 971, 973, 975, 981, 983, 985, 987, 989, 991, 993, 995, 997, 999, 1010 or 1013. In some embodiments, the SpyCas9 nickase is encoded by a nucleotide sequence that has at least 90%, 91%, 92%, 93%, 94%, 95%, 96%, 97%, 98%, 99%, or 100% identity to SEQ ID NO: 1010 or 1013. In some embodiments, the SpyCas9 nickase is encoded by a nucleotide sequence of any one of SEQ ID NOs: 971, 973, 975, 981, 983, 985, 987, 989, 991, 993, 995, 997, 999, 1010 or 1013. In some embodiments, the SpyCas9 nickase is encoded by a nucleotide sequence of SEQ ID NO: 1010 or 1013. In some embodiments, the nucleotide sequence comprises an ORF that has at least 90%, 91%, 92%, 93%, 94%, 95%, 96%, 97%, 98%, 99%, or 100% identity to any one of SEQ ID NOs: 1013, 971, 973, 975, 981, 983, 985, 987, 989, 991, 993, 995, 997, 999. In some embodiments, the nucleotide sequence comprises an ORF of any one of SEQ ID NOs: 1013, 971, 973, 975, 981, 983, 985, 987, 989, 991, 993, 995, 997, 999. In some embodiments, a nucleic acid (e.g., mRNA) encoding a SpyCas9 nickase is provided, the nucleic acid comprising an ORF that has at least 90%, 91%, 92%, 93%, 94%, 95%, 96%, 97%, 98%, 99%, or 100% identity to any one of SEQ ID NOs: 1013, 971, 973, 975, 981, 983, 985, 987, 989, 991, 993, 995, 997, 999; or an ORF of any one of SEQ ID NOs: 1013, 971, 973, 975, 981, 983, 985, 987, 989, 991, 993, 995, 997, 999.

In some embodiments, the amino acid sequence of the SpyCas9 nickase lacks an N-terminal methionine, for instance, where the SpyCas9 nickase is comprised within a fusion protein. In some embodiments, the amino acid sequence of the SpyCas9 nickase lacks the N-terminal methionine of any one of SEQ ID NOs: 972, 974, 976, 982, 984, 986, 988, 990, 992, 994, 996, 998, 1000, and 1014, for instance, where the SpyCas9 nickase is comprised within a fusion protein. In other embodiments, the amino acid sequence of the SpyCas9 nickase comprises any one of SEQ ID NOs: 972, 974, 976, 982, 984, 986, 988, 990, 992, 994, 996, 998, 1000, and 1014, including or lacking the N-terminal methionine. In some embodiments, the nucleotide sequence encoding the SpyCas9 nickase lacks a start codon or stop codon, for instance, where the SpyCas9 nickase is comprised within a fusion protein. In some embodiments, the nucleotide sequence encoding the SpyCas9 nickase lacks the start codon or stop codon of any one of SEQ ID NOs: 971, 973, 975, 981, 983, 985, 987, 989, 991, 993, 995, 997, 999, 1010 and 1013, for instance, where the SpyCas9 nickase is comprised within a fusion protein. In other embodiments, the nucleotide sequence encoding the SpyCas9 nickase comprises any one of SEQ ID NOs: 971, 973, 975, 981, 983, 985, 987, 989, 991, 993, 995, 997, 999, 1010 and 1013, including or lacking the start codon or stop codon.

In some embodiments, the SpyCas9 nickase generates a nick on the non-target strand of the DNA duplex target nucleic acid, i.e., the SpyCas9 nickase generates a nick on the strand opposite to the strand that hybridizes to the spacer. In some embodiments, the SpyCas9 nickase generates a nick on the same strand of the DNA duplex target nucleic acid as a SpyCas9 nuclease comprising a catalytically inactive HNH domain. In some embodiments, the SpyCas9 nickase comprises a catalytically inactive HINH domain. In some embodiments, the SpyCas9 nickase comprises a D839K, D839Q, D839N, D839A, H840S, H840T, H840Q, H840D, H840R, H840K, H840Y, H840N, N863S, N863T, N863A, N863E, or K866A mutation within its HNH domain. In some embodiments, the SpyCas9 nickase comprises an H840A point mutation. In some embodiments, wherein the SpyCas9 nuclease is a nickase, the SpyCas9 nickase comprises an amino acid sequence of any one of SEQ ID NOs: 1014, 972, 974, 976, 982, 984, 986, 988, 990, 992, 994, 996, 998, 1000.

In some embodiments, the SpyCas9 nickase comprises an amino acid sequence that has at least 90%, 91%, 92%, 93%, 94%, 95%, 96%, 97%, 98%, 99%, or 100% identity to any one of any one of SpyCas9 nickases listed in Table 6. In some embodiments, a nucleotide sequence encoding an SpyCas9 nickase comprises an ORF that has at least 90%, 91%, 92%, 93%, 94%, 95%, 96%, 97%, 98%, 99%, or 100% identity to any one of the ORF sequences encoding a SpyCas9 nickase listed in Table 6. It is understood that if a DNA sequence (comprising Ts) is referenced with respect to an RNA, then Ts should be replaced with Us (which may be modified or unmodified depending on the context), and vice versa.

In some embodiments, a SpyCas9 nickase described herein is operably linked to a DNA-dependent DNA polymerase. In some embodiments, the SpyCas9 nickase and the DNA-dependent DNA polymerase are linked in a fusion protein. In such a fusion protein, the SpyCas9 nickase can be N-terminal of the DNA-dependent DNA polymerase, in some embodiments. In other embodiments, the DNA-dependent DNA polymerase can be N-terminal of the SpyCas9 nickase. In some embodiments, the fusion protein is expressed from a single mRNA. In other embodiments, the fusion protein can be expressed from multiple mRNAs, and linked via intein splicing, as described herein.

### DNA-Dependent DNA Polymerase

In some embodiments, the DRS bound to the non-target strand of the DNA duplex target nucleic acid forms a binding site for a DNA-dependent DNA polymerase.

In some embodiments, the DNA-dependent DNA polymerase comprises human DNA polymerase K, cynomolgus DNA polymerase K, mouse DNA polymerase K, human DNA polymerase θ, human DNA polymerase N, E. coli DNA polymerase I, phage T5 DNA polymerase, or phage Phi29 DNA polymerase. In some embodiments, the DNA-dependent DNA polymerase is human DNA polymerase K. In some embodiments, the DNA-dependent DNA polymerase is phage T5 DNA polymerase. In some embodiments, the DNA-dependent DNA polymerase is not phage Phi29 DNA polymerase. In some embodiments, the DNA-dependent DNA polymerase comprises a substitution mutation (such as a point mutation). In some embodiments, the DNA-dependent DNA polymerase comprises a substitution mutation (such as a point mutation) within an exonuclease domain. In some embodiments, the substitution or deletion is present in a 3′ to 5′ exonuclease domain. Accordingly, in some embodiments, the DNA-dependent DNA polymerase used in the compositions, systems, and methods provided herein lacks a 3′ to 5′ exonuclease activity. In some embodiments, the DNA-dependent DNA polymerase comprises a deletion. In some embodiments, the deletion is within an exonuclease domain. In some embodiments, the deletion comprises the entire exonuclease domain. In some embodiments, the deletion is at the N-terminus, the C-terminus, or both the N-terminus and the C-terminus. In some embodiments, the deletion is an internal deletion.

In some embodiments, the DNA-dependent DNA polymerase comprises a human DNA-dependent DNA polymerase. In some embodiments, the human DNA-dependent DNA polymerase is DNA polymerase K, DNA polymerase θ, or DNA polymerase N.

In some embodiments, the DNA-dependent DNA polymerase comprises an amino acid sequence that has at least 90%, 91%, 92%, 93%, 94%, 95%, 96%, 97%, 98%, 99%, or 100% identity to any one of any one of DNA-dependent DNA polymerases listed in Table 6. In some embodiments, a nucleotide sequence encoding a DNA-dependent DNA polymerase comprises an ORF that has at least 90%, 91%, 92%, 93%, 94%, 95%, 96%, 97%, 98%, 99%, or 100% identity to any one of the ORF sequences encoding a DNA-dependent DNA polymerase listed in Table 6. It is understood that if a DNA sequence (comprising Ts) is referenced with respect to an RNA, then Ts should be replaced with Us (which may be modified or unmodified depending on the context), and vice versa.

In some embodiments, the DNA-dependent DNA polymerase further comprises a heterodimerization domain. In some embodiments, the heterodimerization domain comprises a coiled coil heterodimerization domain, e.g., a self-assembling coiled coil heterodimerization domain. In some embodiments, the heterodimerization domain comprises an EV, EI, KV, or KI domain, which may form a heterodimer with a corresponding self-assembling coiled coil heterodimerization domain. In some embodiments, the heterodimerization domain comprises an EI or KI domain. In some embodiments, the heterodimerization domain comprises an EI domain. In some embodiments, the heterodimerization domain comprises a KI domain. In some embodiments, the heterodimerization domain is SEQ ID NO: 1589, SEQ ID NO: 1593, or SEQ ID NO: 1595.

In some embodiments, the DNA-dependent DNA polymerase is a human DNA polymerase K. In some embodiments, the DNA-dependent DNA polymerase is a human DNA polymerase K comprising an amino acid sequence that has at least 90%, 91%, 92%, 93%, 94%, 95%, 96%, 97%, 98%, 99%, or 100% identity to SEQ ID NO: 1021. In some embodiments, the DNA-dependent DNA polymerase is a human DNA polymerase K comprising an amino acid sequence of SEQ ID NO: 1021. A human polymerase K lacks an endogenous 3′ to 5′ exonuclease activity. Accordingly, in some embodiments, the DNA-dependent polymerase is a human PolK lacking 3′ to 5′ exonuclease activity. In some embodiments, the DNA-dependent DNA polymerase K comprises 3′ exonuclease activity. In some embodiments, the DNA-dependent DNA polymerase is a human DNA polymerase K comprising: one or more point mutations relative to SEQ ID NO: 1021; or a deletion relative to SEQ ID NO: 1021. In some embodiments, the DNA-dependent DNA polymerase is a human DNA polymerase K comprising: a point mutation at one or more of positions 411, 412, 415, 423, and 507 relative to SEQ ID NO: 1021; or a deletion of one or more of amino acid residues 1-18 and 527-870 relative to SEQ ID NO: 1021. A human DNA polymerase K lacking amino acid residues 1-18 and 527-870 relative to SEQ ID NO: 1021, e.g., SEQ ID NO: 1206, may be referred to herein as “polK (19-526).” In some embodiments, the DNA-dependent DNA polymerase is a human DNA polymerase K comprising point mutations at positions 411 and 412 relative to SEQ ID NO: 1021. In some embodiments, the point mutations comprise G411R and E412V (i.e., alternatively referred to herein as GE411-412RV mutations). A human DNA polymerase K comprising GE411-412RV relative to SEQ ID NO: 1021 may be referred to herein as “polK (1-526, GE411-412RV)”. In some embodiments, the DNA-dependent DNA polymerase is a human DNA polymerase K comprising a point mutation at position 415 relative to SEQ ID NO: 1021. In some embodiments, the point mutation comprises S415A. In some embodiments, the DNA-dependent DNA polymerase is a human DNA polymerase K comprising a point mutation at position 423 relative to SEQ ID NO: 1021. In some embodiments, the point mutation comprises S423R. In some embodiments, the DNA-dependent DNA polymerase is a human DNA polymerase K comprising a point mutation at position 507 relative to SEQ ID NO: 1021. In some embodiments, the point mutation comprises R507A or R507G. In some embodiments, the DNA-dependent DNA polymerase is a human DNA polymerase K comprising a deletion of amino acid residues 527-870 relative to SEQ ID NO: 1021. In some embodiments, the DNA-dependent DNA polymerase is a human DNA polymerase K comprising a deletion of amino acid residues 1-18 and 527-870 relative to SEQ ID NO: 1021, i.e., in some embodiments, the human DNA polymerase K comprises amino acids 19-526 relative to SEQ ID NO: 1021. In some embodiments, the DNA-dependent DNA polymerase is a human DNA polymerase K comprising point mutations at positions 411 and 412 and a deletion of amino acid residues 527-870 relative to SEQ ID NO: 1021. In some embodiments, the DNA-dependent DNA polymerase is a human DNA polymerase K comprising G411R and E412V and a deletion of amino acid residues 527-870 relative to SEQ ID NO: 1021. In some embodiments, the DNA-dependent DNA polymerase is a human DNA polymerase K comprising point mutations at positions 411 and 412 and a deletion of amino acid residues 1-18 and 527-870 relative to SEQ ID NO: 1021. In some embodiments, the DNA-dependent DNA polymerase is a human DNA polymerase K comprising G411R and E412V and a deletion of amino acid residues 1-18 and 527-870 relative to SEQ ID NO: 1021, i.e., in some embodiments, the human DNA polymerase K comprising point mutations comprising G411R and E412V and comprising amino acids 19-526 relative to SEQ ID NO: 1021 may be referred to herein as “polK (1-526, GE411-412RV)”. In some embodiments, the human DNA polymerase K does not comprise a point mutation that abrogates polymerase activity. In some embodiments, the human DNA polymerase K does not comprise a point mutation in R507.

In some embodiments, the amino acid sequence of the human DNA polymerase K lacks an N-terminal methionine, where the human DNA polymerase K is comprised within a fusion protein. In some embodiments, the amino acid sequence of the human DNA polymerase K lacks the N-terminal methionine of SEQ ID NO: 1021, for instance, where the human DNA polymerase K is comprised within a fusion protein. In other embodiments, the amino acid sequence of the human DNA polymerase K comprises SEQ ID NO: 1021, including or lacking the N-terminal methionine.

In some embodiments, the DNA-dependent DNA polymerase is a human DNA polymerase K that is encoded by a nucleotide sequence that has at least 90%, 91%, 92%, 93%, 94%, 95%, 96%, 97%, 98%, 99%, or 100% identity to SEQ ID NO: 1020. In some embodiments, a nucleic acid (e.g., mRNA) comprising an open reading frame (ORF) encoding a human DNA polymerase K is provided, the nucleic acid comprising a nucleotide sequence comprising at least 90%, 91%, 92%, 93%, 94%, 95%, 96%, 97%, 98%, 99%, or 100% identity to SEQ ID NO: 1020.

In some embodiments, the present disclosure provides for a polypeptide comprising a human DNA polymerase K comprising GE411-412RV mutations relative to SEQ ID NO: 1021, the polypeptide comprising the amino acid sequence of SEQ ID NO: 1265. In some embodiments, the present disclosure provides for a polypeptide comprising a human DNA polymerase K comprising GE411-412RV mutations relative to SEQ ID NO: 1021 and comprising amino acids 19-526 relative to SEQ ID NO: 1021, the polypeptide comprising the amino acid sequence of SEQ ID NO: 1019.

In some embodiments, the present disclosure provides for a nucleic acid (e.g., mRNA) comprising an open reading frame (ORF) encoding a human DNA polymerase K comprising GE411-412RV mutations relative to SEQ ID NO: 1021, the nucleic acid comprising the nucleotide sequence of SEQ ID NO: 1264. In some embodiments, the present disclosure provides for a nucleic acid (e.g., mRNA) comprising an open reading frame (ORF) encoding a human DNA polymerase K the present disclosure provides for a polypeptide comprising a human DNA polymerase K comprising GE411-412RV mutations relative to SEQ ID NO: 1021 and comprising amino acids 19-526 relative to SEQ ID NO: 1021, the nucleic acid comprising the nucleotide sequence of SEQ ID NO: 1018.

In some embodiments, the nucleotide sequence encoding the human DNA polymerase K lacks a start codon or stop codon, for instance, where the human DNA polymerase K is comprised within a fusion protein. In some embodiments, the nucleotide sequence encoding the human DNA polymerase K lacks the start codon or stop codon of SEQ ID NO: 1020, for instance, where the human DNA polymerase K is comprised within a fusion protein. In other embodiments, the nucleotide sequence of the human DNA polymerase K comprises SEQ ID NO: 1020, including or lacking the start codon or stop codon.

In some embodiments, the DNA-dependent DNA polymerase is a cynomolgus DNA polymerase K. In some embodiments, the DNA-dependent DNA polymerase is a cynomolgus DNA polymerase K comprising an amino acid sequence that has at least 90%, 91%, 92%, 93%, 94%, 95%, 96%, 97%, 98%, 99%, or 100% identity to SEQ ID NO: 1261. In some embodiments, the DNA-dependent DNA polymerase is a cynomolgus DNA polymerase K lacking 3′ to 5′ exonuclease activity. In some embodiments, the DNA-dependent DNA polymerase is a cynomolgus DNA polymerase K comprising: one or more point mutations relative to SEQ ID NO: 1261; or a deletion relative to SEQ ID NO: 1261. In some embodiments, the DNA-dependent DNA polymerase is a cynomolgus DNA polymerase K comprising: a point mutation at one or both of positions 411 or 412 relative to SEQ ID NO: 1261; or a deletion of amino acid residues 527-863 relative to SEQ ID NO: 1261. In some embodiments, the DNA-dependent DNA polymerase is a cynomolgus DNA polymerase K comprising point mutations at positions 411 and 412 relative to SEQ ID NO: 1261. In some embodiments, the point mutations comprise G411R and E412V (i.e., alternatively referred to elsewhere herein as GE411-412RV mutations). In some embodiments, the DNA-dependent DNA polymerase is a cynomolgus DNA polymerase K comprising a deletion of amino acid residues 527-863 relative to SEQ ID NO: 1261. In some embodiments, the DNA-dependent DNA polymerase is a cynomolgus DNA polymerase K comprising point mutations at positions 411 and 412 and a deletion of amino acid residues 527-863 relative to SEQ ID NO: 1261. In some embodiments, the DNA-dependent DNA polymerase is a cynomolgus DNA polymerase K comprising G411R and E412V and a deletion of amino acid residues 527-863 relative to SEQ ID NO: 1261.

In some embodiments, the amino acid sequence of the cynomolgus DNA polymerase K lacks an N-terminal methionine, for instance, where the cynomolgus DNA polymerase K is comprised within a fusion protein. In some embodiments, the amino acid sequence of the cynomolgus DNA polymerase K lacks the N-terminal methionine of SEQ ID NO: 1261, for instance, where the cynomolgus DNA polymerase K is comprised within a fusion protein. In other embodiments, the amino acid sequence of the cynomolgus DNA polymerase comprises SEQ ID NO: 1261, including or lacking the N-terminal methionine.

In some embodiments, the DNA-dependent DNA polymerase is a cynomolgus DNA polymerase K that is encoded by a nucleotide sequence that has at least 90%, 91%, 92%, 93%, 94%, 95%, 96%, 97%, 98%, 99%, or 100% identity to SEQ ID NO: 1260. In some embodiments, a nucleic acid (e.g., mRNA) encoding the cynomolgus DNA polymerase K is provided, the nucleic acid comprising a nucleotide sequence that has at least 90%, 91%, 92%, 93%, 94%, 95%, 96%, 97%, 98%, 99%, or 100% identity to SEQ ID NO: 1260.

In some embodiments, the nucleotide sequence encoding the cynomolgus DNA polymerase K lacks a start codon or stop codon, for instance, where the cynomolgus DNA polymerase K is comprised within a fusion protein. In some embodiments, the nucleotide sequence encoding the cynomolgus DNA polymerase K lacks the start codon or stop codon of SEQ ID NO: 1260, for instance, where the cynomolgus DNA polymerase K is comprised within a fusion protein. In other embodiments, the nucleotide sequence encoding the cynomolgus DNA polymerase K comprises SEQ ID NO: 1260, including or lacking the start codon or stop codon.

In some embodiments, the DNA-dependent DNA polymerase is a mouse DNA polymerase K. In some embodiments, the DNA-dependent DNA polymerase is a mouse DNA polymerase K comprising an amino acid sequence that has at least 90%, 91%, 92%, 93%, 94%, 95%, 96%, 97%, 98%, 99%, or 100% identity to SEQ ID NO: 1263. In some embodiments, the DNA-dependent DNA polymerase is a mouse DNA polymerase K comprising: one or more point mutations relative to SEQ ID NO: 1263; or a deletion relative to SEQ ID NO: 1263. In some embodiments, the DNA-dependent DNA polymerase is a mouse DNA polymerase K comprising: a point mutation at one or both of positions 410 or 411 relative to SEQ ID NO: 1263; or a deletion of amino acid residues 526-852 relative to SEQ ID NO: 1263. In some embodiments, the DNA-dependent DNA polymerase is a mouse DNA polymerase K comprising point mutations at positions 410 and 411 relative to SEQ ID NO: 1263. In some embodiments, the point mutations comprise G410R and E411V. In some embodiments, the DNA-dependent DNA polymerase is a mouse DNA polymerase K comprising a deletion of amino acid residues 526-852 relative to SEQ ID NO: 1263. In some embodiments, the DNA-dependent DNA polymerase is a mouse DNA polymerase K comprising point mutations at positions 410 and 411 and a deletion of amino acid residues 526-852 relative to SEQ ID NO: 1263. In some embodiments, the DNA-dependent DNA polymerase is a mouse DNA polymerase K comprising G410R and E411V and a deletion of amino acid residues 526-852 relative to SEQ ID NO: 1263.

In some embodiments, the amino acid sequence of the mouse DNA polymerase K lacks an N-terminal methionine, for instance, where the mouse DNA polymerase K is comprised within a fusion protein. In some embodiments, the amino acid sequence of the mouse DNA polymerase K lacks the N-terminal methionine of SEQ ID NO: 1263, for instance, where the cynomolgus DNA polymerase K is comprised within a fusion protein. In other embodiments, the amino acid sequence of the mouse DNA polymerase K comprises SEQ ID NO: 1263, including or lacking the N-terminal methionine.

In some embodiments, the DNA-dependent DNA polymerase is a mouse DNA polymerase K that is encoded by a nucleotide sequence that has at least 90%, 91%, 92%, 93%, 94%, 95%, 96%, 97%, 98%, 99%, or 100% identity to SEQ ID NO: 1262. In some embodiments, a nucleic acid (e.g., mRNA) encoding the DNA-dependent DNA polymerase K is provided, the nucleic acid comprising a nucleotide sequence that has at least 90%, 91%, 92%, 93%, 94%, 95%, 96%, 97%, 98%, 99%, or 100% identity to SEQ ID NO: 1262.

In some embodiments, the nucleotide sequence encoding the mouse DNA polymerase K lacks a start codon or stop codon, for instance, where the mouse DNA polymerase K is comprised within a fusion protein. In some embodiments, the nucleotide sequence encoding the mouse DNA polymerase K lacks the start codon or stop codon of SEQ ID NO: 1262, for instance, where the mouse DNA polymerase K is comprised within a fusion protein. In other embodiments, the nucleotide sequence encoding the mouse DNA polymerase K comprises SEQ ID NO: 1262, including or lacking the start codon or stop codon.

In some embodiments, the DNA-dependent DNA polymerase is human DNA polymerase θ. In some embodiments, the DNA-dependent DNA polymerase is a human DNA polymerase θ comprising an amino acid sequence that has at least 90%, 91%, 92%, 93%, 94%, 95%, 96%, 97%, 98%, 99%, or 100% identity to SEQ ID NO: 1076 or 1081. In some embodiments, the DNA-dependent DNA polymerase is a human DNA polymerase θ comprising an amino acid sequence of SEQ ID NOs: 1076. In some embodiments, the DNA-dependent DNA polymerase is a human DNA polymerase θ comprising a point mutation or deletion, optionally of amino acid residues 1-1818 relative to SEQ ID NO: 1076, i.e., in some embodiments, the human DNA polymerase θ comprises amino acids 1819-2590 of SEQ ID NO: 1076. In some embodiments, the DNA-dependent DNA polymerase is a human DNA polymerase θ comprising a deletion of amino acid residues 1-1818 relative to SEQ ID NO: 1076. In some embodiments, the DNA-dependent DNA polymerase is a human DNA polymerase θ comprising SEQ ID NO: 1081. In some embodiments, the DNA-dependent DNA polymerase is a human DNA polymerase θ that is encoded by a nucleotide sequence that has at least 90%, 91%, 92%, 93%, 94%, 95%, 96%, 97%, 98%, 99%, or 100% identity to SEQ ID NO: 1075 or 1080. In some embodiments, a nucleic acid (e.g., mRNA) encoding a human DNA polymerase θ is provided, the nucleic acid comprising a nucleotide sequence that has at least 90%, 91%, 92%, 93%, 94%, 95%, 96%, 97%, 98%, 99%, or 100% identity to SEQ ID NO: 1075 or 1080.

In some embodiments, the amino acid sequence of the human DNA polymerase θ lacks an N-terminal methionine, for instance, where the human DNA polymerase θ is comprised within a fusion protein. In some embodiments, the amino acid sequence of the human DNA polymerase θ lacks the N-terminal methionine of SEQ ID NO: 1076 or 1081, for instance, where the human DNA polymerase θ is comprised within a fusion protein. In other embodiments, the amino acid sequence of the human DNA polymerase θ comprises SEQ ID NO: 1076 or 1081, including or lacking the N-terminal methionine. In some embodiments, the nucleotide sequence of the human DNA polymerase θ lacks a start codon or stop codon, for instance, where the human DNA polymerase θ is comprised within a fusion protein. In some embodiments, the nucleotide sequence encoding the human DNA polymerase θ lacks the start codon or stop codon of SEQ ID NO: 1075 or 1080, for instance, where the human DNA polymerase θ is comprised within a fusion protein. In other embodiments, the nucleotide sequence encoding the human DNA polymerase θ comprises SEQ ID NO: 1075 or 1080, including or lacking the start codon or stop codon.

In some embodiments, the DNA-dependent DNA polymerase is human DNA polymerase N. In some embodiments, the DNA-dependent DNA polymerase is a human DNA polymerase N comprising an amino acid sequence that has at least 90%, 91%, 92%, 93%, 94%, 95%, 96%, 97%, 98%, 99%, or 100% identity to SEQ ID NO: 1088. In some embodiments, the DNA-dependent DNA polymerase is a human DNA polymerase N comprising an amino acid sequence of SEQ ID NO: 1088. In some embodiments, the DNA-dependent DNA polymerase is a human DNA polymerase N that is encoded by a nucleotide sequence that has at least 90%, 91%, 92%, 93%, 94%, 95%, 96%, 97%, 98%, 99%, or 100% identity to any one of SEQ ID NOs: 1087, 1089, or 1090. In some embodiments, a nucleic acid (e.g., mRNA) encoding a human DNA polymerase N is provided, the nucleic acid comprising a nucleotide sequence that has at least 90%, 91%, 92%, 93%, 94%, 95%, 96%, 97%, 98%, 99%, or 100% identity to SEQ ID NOs: 1087, 1089, or 1090.

In some embodiments, the amino acid sequence of the human DNA polymerase N lacks an N-terminal methionine, for instance, where the human DNA polymerase N is comprised within a fusion protein. In some embodiments, the amino acid sequence of the human DNA polymerase N lacks the N-terminal methionine of SEQ ID NO: 1088, for instance, where the human DNA polymerase N is comprised within a fusion protein. In other embodiments, the amino acid sequence of the human DNA polymerase N comprises SEQ ID NO: 1088, including or lacking the N-terminal methionine. In some embodiments, the nucleotide sequence encoding the human DNA polymerase N lacks a start codon or stop codon, for instance, where the human DNA polymerase N is comprised within a fusion protein. In some embodiments, the nucleotide sequence encoding the human DNA polymerase N lacks the start codon or stop codon of SEQ ID NO: 1087, 1089, or 1090, for instance, where the human DNA polymerase N is comprised within a fusion protein. In other embodiments, the nucleotide sequence encoding the human DNA polymerase N comprises any one of SEQ ID NOs: 1087, 1089, and 1090, including or lacking the start codon or stop codon.

In some embodiments, the DNA-dependent DNA polymerase disclosed herein comprises a bacterial DNA-dependent DNA polymerase such as an E. coli DNA-dependent DNA polymerase. In some embodiments, the E. coli DNA-dependent DNA polymerase is DNA polymerase I. In some embodiments, the DNA-dependent DNA polymerase disclosed herein comprises a viral DNA-dependent DNA polymerase such as a phage DNA-dependent DNA polymerase. In some embodiments, the phage DNA-dependent DNA polymerase is T5 DNA polymerase or Phi29 DNA polymerase.

In some embodiments, the DNA-dependent DNA polymerase is an E. coli DNA polymerase I. In some embodiments, the DNA-dependent DNA polymerase is an E. coli DNA polymerase I comprising an amino acid sequence that has at least 90%, 91%, 92%, 93%, 94%, 95%, 96%, 97%, 98%, 99%, or 100% identity to SEQ ID NO: 1099. In some embodiments, the DNA-dependent DNA polymerase is an E. coli DNA polymerase I comprising an amino acid sequence of SEQ ID NO: 1099. In some embodiments, the DNA-dependent DNA polymerase is an E. coli DNA polymerase I that is encoded by a nucleotide sequence that has at least 90%, 91%, 92%, 93%, 94%, 95%, 96%, 97%, 98%, 99%, or 100% identity to SEQ ID NO: 1098. In some embodiments, a nucleic acid (e.g., mRNA) encoding an E. coli DNA polymerase I is provided, the nucleic acid comprising a nucleotide sequence that has at least 90%, 91%, 92%, 93%, 94%, 95%, 96%, 97%, 98%, 99%, or 100% identity to SEQ ID NO: 1098.

In some embodiments, the amino acid sequence of the E. coli DNA polymerase I lacks an N-terminal methionine, for instance, where the E. coli DNA polymerase I is comprised within a fusion protein. In some embodiments, the amino acid sequence of the E. coli DNA polymerase I lacks the N-terminal methionine of SEQ ID NO: 1098, for instance, where the E. coli DNA polymerase I is comprised within a fusion protein. In other embodiments, the amino acid sequence of the E. coli DNA polymerase I comprises SEQ ID NO: 1098, including or lacking the N-terminal methionine.

In some embodiments, the E. coli DNA polymerase I comprises a point mutation or deletion relative to SEQ ID NO: 1099. In some embodiments, the E. coli DNA polymerase I comprises a point mutation in positions 355 or 357 relative to SEQ ID NO: 1099. In some embodiments, the E. coli DNA polymerase I comprises point mutations in both positions 355 and 357 relative to SEQ ID NO: 1099, optionally wherein the point mutations comprise both D355A and E357A. In some embodiments, the E. coli DNA polymerase I comprises a deletion within its exonuclease domain relative to SEQ ID NO: 1099. In some embodiments, the E. coli DNA polymerase I comprises a deletion of the entire exonuclease domain relative to SEQ ID NO: 1099. In some embodiments, the deletion abolishes some or all of the catalytic activity of the exonuclease domain.

In some embodiments, the nucleotide sequence of the E. coli DNA polymerase I lacks a start codon or stop codon, for instance, where the E. coli DNA polymerase I is comprised within a fusion protein. In some embodiments, the nucleotide sequence encoding the E. coli DNA polymerase I lacks the start codon or stop codon of SEQ ID NO: 1099, for instance, where the E. coli DNA polymerase I is comprised within a fusion protein. In other embodiments, the nucleotide sequence encoding the E. coli DNA polymerase I comprises SEQ ID NO: 1099, including or lacking the start codon or stop codon.

In some embodiments, the DNA-dependent DNA polymerase is an E. coli DNA polymerase I comprising a Klenow fragment. In some embodiments, the DNA-dependent DNA polymerase is an E. coli DNA polymerase I comprising a deletion of amino acid residues 1-323 relative to SEQ ID NO: 1099. In some embodiments, the DNA-dependent DNA polymerase is an E. coli DNA polymerase I comprising D355A, E357A, and a deletion of amino acid residues 1-323 relative to SEQ ID NO: 1099 In some embodiments, the DNA-dependent DNA polymerase is an E. coli DNA polymerase I comprising a Klenow fragment that has at least 90%, 91%, 92%, 93%, 94%, 95%, 96%, 97%, 98%, 99%, or 100% identity to SEQ ID NO: 1104 or 1109. In some embodiments, the DNA-dependent DNA polymerase is an E. coli DNA polymerase I comprising a Klenow fragment that has the amino acid sequence of SEQ ID NO: 1104 or 1109. In some embodiments, the DNA-dependent DNA polymerase is an E. coli DNA polymerase I comprising a Klenow fragment that is encoded by a nucleotide sequence that has at least 90%, 91%, 92%, 93%, 94%, 95%, 96%, 97%, 98%, 99%, or 100% identity to SEQ ID NO: 1103 or 1108. In some embodiments, a nucleic acid (e.g., mRNA) encoding an E. coli DNA polymerase I comprising a Klenow fragment is provided, the nucleic acid comprising a nucleotide sequence that has at least 90%, 91%, 92%, 93%, 94%, 95%, 96%, 97%, 98%, 99%, or 100% identity to SEQ ID NO: 1103 or 1108.

In some embodiments, the amino acid sequence of the E. coli DNA polymerase I lacks an N-terminal methionine, for instance, where the E. coli DNA polymerase I is comprised within a fusion protein. In some embodiments, the amino acid sequence of the E. coli DNA polymerase I lacks the N-terminal methionine of SEQ ID NO: 1104 or 1109, for instance, where the E. coli DNA polymerase I is comprised within a fusion protein. In other embodiments, the amino acid sequence of the E. coli DNA polymerase I comprises SEQ ID NO: 1104 or 1109, including or lacking the N-terminal methionine. In some embodiments, the nucleotide sequence encoding the E. coli DNA polymerase I lacks a start codon or stop codon, for instance, where the E. coli DNA polymerase I is comprised within a fusion protein. In some embodiments, the nucleotide sequence encoding the E. coli DNA polymerase I lacks the start codon or stop codon of SEQ ID NO: 1103 or 1108, for instance, where the E. coli DNA polymerase I is comprised within a fusion protein. In other embodiments, the nucleotide sequence encoding the E. coli DNA polymerase I comprises SEQ ID NO: 1103 or 1108, including or lacking the start codon or stop codon.

In some embodiments, the DNA-dependent DNA polymerase is a phage T5 DNA polymerase. In some embodiments, the DNA-dependent DNA polymerase is a phage T5 DNA polymerase comprising an amino acid sequence that has at least 90%, 91%, 92%, 93%, 94%, 95%, 96%, 97%, 98%, 99%, or 100% identity to any one of SEQ ID NO: 1116 or 1121. In some embodiments, the DNA-dependent DNA polymerase is a phage T5 DNA polymerase comprising an amino acid sequence of SEQ ID NO: 1116. In some embodiments, the DNA-dependent DNA polymerase is a phage T5 DNA polymerase that lacks 3′ to 5′ exonuclease activity. In some embodiments, the DNA-dependent DNA polymerase is a phage T5 DNA polymerase comprising a deletion relative to SEQ ID NO: 1116; or one or more point mutations relative to SEQ ID NO: 1116. In some embodiments, the DNA-dependent DNA polymerase is a phage T5 DNA polymerase comprising a deletion of one or more of amino acid residues 1-30 and the exonuclease domain relative to SEQ ID NO: 1116. In some embodiments, the DNA-dependent DNA polymerase is a phage T5 DNA polymerase comprising point mutations at one or more of positions 164, 166, 425, and 593 relative to SEQ ID NO: 1116. In some embodiments, the DNA-dependent DNA polymerase is a phage T5 DNA polymerase comprising point mutations at positions 164 and 166 relative to SEQ ID NO: 1116. In some embodiments, the point mutations comprise D164A and E166A. In some embodiments, the DNA-dependent DNA polymerase comprises the sequence of SEQ ID NO: 1121. In some embodiments, the DNA-dependent DNA polymerase is a phage T5 DNA polymerase comprising point mutations at positions 164, 166, and 425 relative to SEQ ID NO: 1116. In some embodiments, the point mutations comprise D164A, E166A, and K425I. In some embodiments, the DNA-dependent DNA polymerase is a phage T5 DNA polymerase comprising point mutations at positions 164, 166, and 593 relative to SEQ ID NO: 1116. In some embodiments, the point mutations comprise D164A, E166A, and A593R. In some embodiments, the DNA-dependent DNA polymerase is a phage T5 DNA polymerase comprising a deletion of amino acid residues 1-30 and point mutations at positions 164 and 166 relative to SEQ ID NO: 1116. In some embodiments, the DNA-dependent DNA polymerase is a phage T5 DNA polymerase comprising D164A, E166A, and a deletion of amino acid residues 1-30. In some embodiments, the DNA-dependent DNA polymerase is a phage T5 DNA polymerase that does not comprise a mutation that abrogates polymerase activity. In some embodiments, the DNA-dependent DNA polymerase as a phage T5 DNA polymerase that does not comprise a deletion of one or more of amino acid residues 1-30, 793-855, or 832-855. In some embodiments, the DNA-dependent DNA polymerase is a phage T5 DNA polymerase that is encoded by a nucleotide sequence that has at least 90%, 91%, 92%, 93%, 94%, 95%, 96%, 97%, 98%, 99%, or 100% identity to any one of SEQ ID NOs: 1115 or 1120. In some embodiments, a nucleic acid (e.g., mRNA) encoding a phage T5 DNA polymerase comprising D164A and E166A mutations is provided, the nucleic acid comprising a nucleotide sequence that has at least 90%, 91%, 92%, 93%, 94%, 95%, 96%, 97%, 98%, 99%, or 100% identity to any one of SEQ ID NOs: 1115 or 1120.

In some embodiments, the present disclosure provides for a polypeptide comprising a phage T5 DNA polymerase comprising D164A and E166A mutations relative to SEQ ID NO: 1116 (also referred to as “T5Pol(D164A, E166A)” elsewhere herein), the polypeptide comprising an amino acid sequence having at least 90%, 91%, 92%, 93%, 94%, 95%, 96%, 97%, 98%, 99%, or 100% identity to SEQ ID NO: 1121. In some embodiments, the present disclosure provides for a nucleic acid (e.g., mRNA) comprising an open reading frame (ORF) encoding a phage T5 DNA polymerase comprising D164A and E166A mutations relative to SEQ ID NO: 1116 (also referred to as “T5Pol(D164A,E166A)” elsewhere herein), the ORF having at least 90%, 91%, 92%, 93%, 94%, 95%, 96%, 97%, 98%, 99%, or 100% identity to SEQ ID NO: 1120.

In some embodiments, the amino acid sequence of the phage T5 DNA polymerase lacks an N-terminal methionine, for instance, where the phage T5 DNA polymerase is comprised within a fusion protein. In some embodiments, the amino acid sequence of the phage T5 DNA polymerase lacks the N-terminal methionine of SEQ ID NO: 1116, for instance, where the phage T5 DNA polymerase is comprised within a fusion protein. In other embodiments, the amino acid sequence of the phage T5 DNA polymerase comprises SEQ ID NO: 1116, including or lacking the N-terminal methionine. In some embodiments, the nucleotide sequence encoding the phage T5 DNA polymerase lacks the start codon or stop codon, for instance, where the phage T5 DNA polymerase is comprised within a fusion protein. In some embodiments, the nucleotide sequence encoding the phage T5 DNA polymerase lacks the start codon or stop codon of SEQ ID NO: 1115 or 1120, for instance, where the phage T5 DNA polymerase is comprised within a fusion protein. In other embodiments, the nucleotide sequence encoding the phage T5 DNA polymerase comprises SEQ ID NO: 1115 or 1120, including or lacking the start codon or stop codon.

In some embodiments, the DNA-dependent DNA polymerase is a phage Phi29 DNA polymerase. In some embodiments, the DNA-dependent DNA polymerase is a phage Phi29 DNA polymerase comprising an amino acid sequence that has at least 90%, 91%, 92%, 93%, 94%, 95%, 96%, 97%, 98%, 99%, or 100% identity to SEQ ID NO: 1184. In some embodiments, the DNA-dependent DNA polymerase is a phage Phi29 DNA polymerase comprising an amino acid sequence of SEQ ID NO: 1184. In some embodiments, the DNA-dependent DNA polymerase is a phage Phi29 DNA polymerase that lacks 3′ to 5′ exonuclease activity. In some embodiments, the DNA-dependent DNA polymerase is a phage Phi29 DNA polymerase comprising one or more point mutations. In some embodiments, the DNA-dependent DNA polymerase is a phage Phi29 DNA polymerase comprising one or more point mutations, optionally at one or more of positions 8, 12, 14, 51, 97, 197, 221, 497, 512, and 526 relative to SEQ ID NO: 1184. In some embodiments, the DNA-dependent DNA polymerase is a phage Phi29 DNA polymerase comprising point mutations at each of positions 8, 51, 97, 197, 221, 497, 512, and 526 relative to SEQ ID NO: 1184. In some embodiments, the DNA-dependent DNA polymerase is a phage Phi29 DNA polymerase comprising M8R, V51A, M97T, G197D, E221K, Q497P, K512E and F526L. In some embodiments, the DNA-dependent DNA polymerase is a phage Phi29 DNA polymerase comprising point mutations at each of positions 8, 12, 14, 51, 97, 197, 221, 497, 512, and 526 relative to SEQ ID NO: 1184. In some embodiments, the DNA-dependent DNA polymerase is a phage Phi29 DNA polymerase comprising M8R, D12A, E14A, V51A, M97T, G197D, E221K, Q497P, K512E, and F526L. In some embodiments, the DNA-dependent DNA polymerase is a phage Phi29 DNA polymerase that is encoded by a nucleotide sequence that has at least 90%, 91%, 92%, 93%, 94%, 95%, 96%, 97%, 98%, 99%, or 100% identity to any one of SEQ ID NOs: 1183 or 1188. In some embodiments, a nucleic acid (e.g., mRNA) encoding a phage Phi29 DNA polymerase is provided, the nucleic acid comprising a nucleotide sequence that has at least 90%, 91%, 92%, 93%, 94%, 95%, 96%, 97%, 98%, 99%, or 100% identity to any one of SEQ ID NOs: 1183 or 1188. In some embodiments, the DNA-dependent DNA polymerase is not a phage Phi29 DNA polymerase.

In some embodiments, the amino acid sequence of the phage Phi29 DNA polymerase lacks an N-terminal methionine, for instance, where the phage Phi29 DNA polymerase is comprised within a fusion protein. In some embodiments, the amino acid sequence of the phage Phi29 DNA polymerase lacks the N-terminal methionine of SEQ ID NO: 1184, for instance, where the phage Phi29 DNA polymerase is comprised within a fusion protein. In other embodiments, the amino acid sequence of the phage Phi29 DNA polymerase comprises SEQ ID NO: 1184, including or lacking the N-terminal methionine. In some embodiments, the nucleotide sequence encoding the phage Phi29 DNA polymerase lacks a start codon or stop codon, for instance, where the phage Phi29 DNA polymerase is comprised within a fusion protein. In some embodiments, the nucleotide sequence encoding the phage Phi29 DNA polymerase lacks the start codon or stop codon of SEQ ID NO: 1183 or 1188, for instance, where the phage Phi29 DNA polymerase is comprised within a fusion protein. In other embodiments, the nucleotide sequence encoding the phage Phi29 DNA polymerase comprises SEQ ID NO: 1183 or 1188, including or lacking the start codon or stop codon.

In some embodiments, the extension activity of the DNA-dependent DNA polymerase is at least 4-fold higher, e.g., at least 10-fold higher, at least 20-fold higher, in the presence of a template sequence consisting of DNA nucleotides than in the presence of a template sequence consisting of RNA nucleotides. In some embodiments, the extension activity of the DNA-dependent DNA polymerase is at least 4-fold higher, e.g., at least 10-fold higher, at least 20-fold higher, in the presence of a template sequence consisting of DNA nucleotides than in the presence of a template sequence consisting of RNA nucleotides.

In some embodiments, the DNA-dependent DNA polymerase does not comprise a mutation in its 3′->5′ exonuclease domain, if present. In some embodiments, the DNA-dependent DNA polymerase comprises a mutation in its 3′->5′ exonuclease domain, if present. In some embodiments the DNA-dependent DNA polymerase is E. coli DNA Polymerase I and comprises mutations in one or more metal-coordinating amino acid residue(s). In some embodiments, the DNA-dependent DNA polymerase is E. coli DNA polymerase I and comprises mutations D355A and E357A. In some embodiments, the E. coli DNA polymerase I comprises the mutation D355A. In some embodiments, the E. coli DNA polymerase I comprises the mutation E357A.

In some embodiments, the polymerase does not include 5 or more, optionally 8 or more point mutations in the amino acid sequence as compared to the wild-type sequence, with point mutations being distinct from truncations. It can be advantageous, in some embodiments, for a DNA-dependent DNA polymerase intended for use in mammalian cells (e.g., human, cyno, or mouse cells) to be active at physiological temperatures, e.g., 37° C. In some embodiments, the wild-type polymerase is more active at 37° C. than 30° C., e.g., the polymerase has at least twice the polymerization rate, preferably at least five times the polymerization rate at 37° C. as compared to 30° C. In some embodiments, the DNA-dependent DNA polymerase present in the compositions, systems, methods, and other aspects provided herein is more active at 37° C. than 30° C., e.g., the polymerase has at least twice the polymerization rate, preferably at least five times the polymerization rate at 37° C. as compared to 30° C. 3′ Extension-Recruiting Domain

In some embodiments, the DNA-dependent DNA polymerase further comprises a 3′ extension-recruiting domain for binding one or more affinity tags. In some embodiments, wherein the affinity tag is an MS2 aptamer, the 3′ extension-recruiting domain is an MS2 coat protein (MCP). In some embodiments, the 3′ extension-recruiting domain is an MS2 coat protein (MCP) with an amino acid sequence that is at least 90%, 91%, 92%, 93%, 94%, 95%, 96%, 97%, 98%, 99%, or 100% identical to SEQ ID NO: 1520 or a nucleotide sequence that is at least 90%, 91%, 92%, 93%, 94%, 95%, 96%, 97%, 98%, 99%, or 100% identical to SEQ ID NO: 1519. In some embodiments, a nuclei acid (e.g., mRNA) encoding an MS2 coat protein (MCP) is provided, the nucleic acid comprising a nucleotide sequence that is at least 90%, 91%, 92%, 93%, 94%, 95%, 96%, 97%, 98%, 99%, or 100% identical to SEQ ID NO: 1519.

In some embodiments, the amino acid sequence of the MCP lacks an N-terminal methionine, for instance, where the MCP is comprised within a fusion protein. In some embodiments, the amino acid sequence of the MCP lacks the N-terminal methionine of SEQ ID NO: 1520, for instance, where the MCP is comprised within a fusion protein. In other embodiments, the amino acid sequence of the MCP comprises SEQ ID NO: 1520, including or lacking the N-terminal methionine. In some embodiments, the nucleotide sequence encoding the MCP lacks a start codon or stop codon, for instance, where the MCP is comprised within a fusion protein. In some embodiments, the nucleotide sequence encoding the MCP lacks the start codon or stop codon of SEQ ID NO: 1519, for instance, where the MCP is comprised within a fusion protein. In other embodiments, the nucleotide sequence encoding the MCP comprises SEQ ID NO: 1519, including or lacking the start codon or stop codon.

In some embodiments, wherein the affinity tag is a PP7 aptamer sequence, the 3′ extension-recruiting domain is a PP7 coat protein. In some embodiments, wherein the affinity tag is a bacterial nucleic acid, the 3′ extension-recruiting domain is a bacterial protein. In some embodiments, wherein the affinity tag is a guide RNA, the 3′ extension-recruiting domain is a portion of a CRISPR protein that binds the guide RNA.

In some embodiments, wherein the affinity tag comprises a small molecule, the 3′ extension-recruiting domain comprises a domain for binding the small molecule. In some embodiments, wherein the small molecule is biotin, desthiobiotin, or digoxigenin and the 3′ extension-recruiting domain is streptavidin.

### Heterodimerization Domain

In some embodiments of the present disclosure, the SpyCas9 nickase and operably linked DNA-dependent DNA polymerase further comprises a heterodimerization domain. In some embodiments of the present disclosure, the SpyCas9 nickase and operably linked DNA-dependent DNA polymerase further comprises a heterodimerization domain or further comprises an additional heterodimerization domain for binding (e.g., coupling, or operably linking) a polypeptide to the DNA-dependent DNA polymerase. In some embodiments, the polypeptide for binding to the DNA-dependent DNA polymerase is not Cas9. In some embodiments, the polypeptide for binding to the DNA-dependent DNA polymerase is an enhancer of template-based genome editing, e.g., a Vpx, BGLF4, M97, KSHV ORF36, or FEN1 protein. In some embodiments, the polypeptide for binding to the DNA-dependent DNA polymerase is a FEN1 protein. In some embodiments, the polypeptide for binding to the DNA-dependent DNA polymerase is a VPX protein. In some embodiments, the polypeptide for binding to the DNA-dependent DNA polymerase is a FEN1 protein and a VPX protein. In some embodiments, the polypeptide for binding to the DNA-dependent DNA polymerase is a VPX protein. In some embodiments, the polypeptide for binding to the DNA-dependent DNA polymerase is a FEN1 protein and a VPX protein. In some embodiments, the heterodimerization domain comprises a coiled coil heterodimerization domain, e.g., a self-assembling coiled coil heterodimerization domain. In some embodiments, the heterodimerization domain comprises an EV, EI, KV, or KI domain to form a heterodimer with a corresponding self-assembling coiled coil heterodimerization domain. In some embodiments, the heterodimerization domain comprises an EI or KI domain. In some embodiments, the heterodimerization domain comprises an EI domain. In some embodiments, the heterodimerization domain comprises a KI domain. In some embodiments, the heterodimerization domain comprises SEQ ID NO: 1589, SEQ ID NO: 1593, or SEQ ID NO: 1595.

### Operable Linkage

In some embodiments of the present disclosure, the SpyCas9 nickase is operably linked to the DNA-dependent DNA polymerase. In some embodiments, the SpyCas9 nickase and DNA-dependent DNA polymerase are operably linked by a covalent linkage between the two enzymes, as described below and elsewhere herein. In some embodiments, the SpyCas9 nickase and DNA-dependent DNA polymerase are operably linked by non-covalent interactions. In some embodiments, the SpyCas9 nickase and DNA-dependent DNA polymerase are operably linked by non-covalent interactions between the SpyCas9 nickase and DNA-dependent DNA polymerase. In some embodiments, the SpyCas9 nickase and DNA-dependent DNA polymerase are operably linked by non-covalent interactions, wherein the SpyCas9 nickase and DNA-dependent DNA polymerase bind separate binding sites on the same template guide RNA. In some embodiments, the SpyCas9 nickase and DNA-dependent DNA polymerase are operably linked by non-covalent interactions, wherein the SpyCas9 nickase binds a first polynucleotide comprising a guide RNA and the DNA-dependent DNA polymerase binds a second polynucleotide comprising a separable 3′ extension, wherein the first and second polynucleotides are separate.

In some embodiments, the SpyCas9 nickase and the DNA-dependent DNA polymerase form a complex bound by an affinity tag. In some embodiments, the affinity tag comprises an MS2 aptamer sequence, e.g., a MS2-loop (alternatively referred to as “MS2 hairpin” or “MS2 RNA loop” elsewhere herein). In some embodiments, the separable 3′ extension of the second polynucleotide comprises a MS2-loop (alternatively referred to elsewhere herein as “MS2 hairpin” or “MS2 RNA loop”). In some embodiments, the separable 3′ extension of the second polynucleotide is a MS2-loop-linked 3′-extension.

### The SpyCas9 Nickase and DNA-Dependent DNA Polymerase May be Covalently Linked

In some embodiments, the SpyCas9 nickase and DNA-dependent DNA polymerase are presented in a single polypeptide. In some embodiments, the SpyCas9 nickase and DNA-dependent DNA polymerase comprises or are comprised in a single polypeptide. In some embodiments, the SpyCas9 nickase and DNA-dependent DNA polymerase comprise a single polypeptide expressed from a single open reading frame (ORF). In some embodiments, wherein the SpyCas9 nickase and DNA-dependent DNA polymerase are covalently linked, the SpyCas9 nickase and DNA-dependent DNA polymerase are expressed from separate ORFs (e.g., separate ORFs in the same mRNA, or separate ORFs provided in separate mRNAs).

In some embodiments, wherein the SpyCas9 nickase and DNA-dependent DNA polymerase are expressed from separate ORFs, a first polypeptide comprising the SpyCas9 nickase and a second polypeptide comprising the DNA-dependent DNA polymerase each comprise an intein. In some embodiments, the covalently linked SpyCas9 nickase and DNA-dependent DNA polymerase are encoded by (a) a first polynucleotide comprising an ORF encoding a polypeptide comprising the SpyCas9 nickase and a first intein and (b) a second polynucleotide comprising an ORF encoding a polypeptide comprising the DNA-dependent DNA polymerase and a second intein capable of binding the first intein (or capable of participating in a trans-splicing reaction with the first intein), wherein the polypeptide comprising the SpyCas9 nickase binds to the polypeptide comprising the DNA-dependent DNA polymerase through intein catalysis.

In some embodiments, wherein the SpyCas9 nickase and DNA-dependent DNA polymerase are expressed from separate ORFs, a first polypeptide comprising the SpyCas9 nickase and a second polypeptide comprising the DNA-dependent DNA polymerase each comprise a portion of an intein (i.e., a split intein). In some embodiments, the covalently linked SpyCas9 nickase and DNA-dependent DNA polymerase are encoded by (a) a first polynucleotide comprising an ORF encoding a polypeptide comprising the SpyCas9 nickase or portion thereof, and a first split intein and (b) a second polynucleotide comprising an ORF encoding a polypeptide comprising the DNA-dependent DNA polymerase, or portion thereof, and a second split intein capable of participating in a trans-splicing reaction with the first split intein, resulting in the formation of a peptide bond between the polypeptide comprising the SpyCas9 nickase and the polypeptide comprising the DNA-dependent DNA polymerase. The trans-splicing reaction thereby results in the formation of a single polypeptide fusion comprising the SpyCas9 nickase and the DNA-dependent DNA polymerase.

In some embodiments, the present disclosure provides for a polynucleotide (e.g., mRNA) comprising an ORF encoding a polypeptide comprising the SpyCas9 nickase or portion thereof and a split intein. In some embodiments, the present disclosure provide for a polynucleotide (e.g., mRNA) comprising an ORF encoding a polypeptide comprising the DNA-dependent DNA polymerase, or portion thereof, and a split intein. In some embodiments, the composition disclosed herein comprises (1) a first polynucleotide (e.g., mRNA) comprising an ORF encoding a polypeptide comprising the SpyCas9 nickase or portion thereof, and a first split intein; and (2) a second polynucleotide (e.g., mRNA) comprising an ORF encoding a polypeptide comprising the DNA-dependent DNA polymerase or portion thereof, and a second split intein. capable of undergoing a trans-splicing reaction with the first split intein. The trans-splicing reaction thereby results in the formation of a single polypeptide comprising the SpyCas9 nickase and the DNA-dependent DNA polymerase.

In some embodiments, the split inteins are an N-split intein polypeptide (N-intein) and a C-split intein polypeptide (C-intein). In some embodiments, the first split intein is an N-intein and the second split intein is a C-intein. For example, a first split intein (N-intein) may be positioned at the C-terminus of the polypeptide comprising the SpyCas9 nickase and a second split intein (C-intein) may be positioned at the N-terminus of the polypeptide comprising the DNA-dependent DNA polymerase. The N-intein and C-intein may then bind together and undergo a trans-splicing reaction resulting in (i) the excision of the first and second split inteins and (ii) the formation of a peptide bond between the polypeptide comprising the SpyCas9 nickase and the polypeptide comprising the DNA-dependent DNA polymerase. In such embodiments, the trans-splicing reaction results in the formation of a single polypeptide fusion comprising from N-terminus to C-terminus a SpyCas9 nickase and a DNA-dependent DNA polymerase.

A person skilled in the art will appreciate that there can be additional amino acids present between the SpyCas9 nickase and the DNA-dependent DNA polymerase, for example, a sequence comprising a peptide linker. Additional amino acids can also result from the residual intein amino acid sequence remaining in the construct after intein splicing. For example, in some embodiments, splicing of CfaN and CfaC split inteins can result in the amino acid sequence “AEYCFN” or “RGKCFN” at the splice site. This residual intein sequence, or “intein scar,” generally does not impact the function of the fusion protein. The intein sequences, and consequently the sequence of the intein scar, can be optimized to improve ligation efficiency, in some embodiments. Other intein scar sequences that may arise following intein fusion include, but are not limited to, the following amino acid sequences: GWNCML, GLTCMY, RGKCWE, GALCWN, AEKCWL, and GDLCKQ.

In some embodiments, the first split intein is a C-intein and the second split intein is an N-intein. For example, a first split intein (C-intein) may be positioned at the N-terminus of the polypeptide comprising the SpyCas9 nickase and a second split intein (N-intein) may be positioned at the C-terminus of the polypeptide comprising the DNA-dependent DNA polymerase. The C-intein and N-intein may then undergo a trans-splicing reaction resulting in (i) the formation of a peptide bond between the polypeptide comprising the SpyCas9 nickase and the polypeptide comprising the DNA-dependent DNA polymerase and (ii) the excision of the first and second split inteins. In such embodiments, the trans-splicing reaction results in the formation of a single polypeptide fusion comprising from N-terminus to C-terminus a DNA-dependent DNA polymerase and a SpyCas9 nickase. In such embodiments, the trans-splicing reaction results in the formation of a single polypeptide fusion comprising from N-terminus to C-terminus a SpyCas9 nickase and a DNA-dependent DNA polymerase.

In some embodiments, the split inteins (e.g., the first and second split inteins) are selected from Cfa, Npu, M86, NpuSsp, gp41-1, gp41-8, NrdJ-1, IMPDH-1, SspDnaX, SspGyrB, Cth-Ter, NrdA-2, Mja-KlbA, RBS1, TE3S11, and Rma. In one embodiment, the first and second split inteins are Cfa split inteins (e.g., CfaN and CfaC split inteins). In one embodiment, the first and second split inteins are M86 split inteins (e.g., M86N and M86C split inteins). In one embodiment, the first and second split inteins are NpuSsp split inteins (e.g., NpuSspN and NpuSspC split inteins). In one embodiment, the first and second split inteins are gp41-1 split inteins (e.g., gp41-1N and gp41-1C split inteins). In one embodiment, the first and second split inteins are gp41-8 split inteins (e.g., gp41-8N and gp41-8C split inteins). In one embodiment, the first and second split inteins are split NrdJ-1 inteins (e.g., NrdJ-1N and NrdJ-1C split inteins). In one embodiment, the first and second split inteins are IMPDH-1 split inteins (e.g., IMPDH-IN and IMPDH-1C split inteins).

In some embodiments, the split inteins are CfaN and CfaC split inteins. In some embodiments, the CfaC split intein comprises an amino acid sequence having at least 90% identity (e.g., at least 90%, 91%, 92%, 93%, 94%, 95%, 96%, 97%, 98%, 99%, or 100%) to SEQ ID NO: 1744 and the CfaN split-intein comprises an amino acid sequence having at least 90% identity (e.g., at least 90%, 91%, 92%, 93%, 94%, 95%, 96%, 97%, 98%, 99%, or 100%) to SEQ ID NO: 1746. In some embodiments, the CfaN split intein comprises the amino acid sequence of SEQ ID NO: 1744 and the CfaC split-intein comprises the amino acid sequence of SEQ ID NO: 1746.

In some embodiments, the split inteins are NpuN and NpuC split inteins. In some embodiments, the NpuN split intein comprises an amino acid sequence having at least 90% identity (e.g., at least 90%, 91%, 92%, 93%, 94%, 95%, 96%, 97%, 98%, 99%, or 100%) to SEQ ID NO: 1753 and the NpuC split-intein comprises an amino acid sequence having at least 90% identity (e.g., at least 90%, 91%, 92%, 93%, 94%, 95%, 96%, 97%, 98%, 99%, or 100%) to SEQ ID NO: 1754. In some embodiments, the NpuN split intein comprises the amino acid sequence of SEQ ID NO: 1753 and the NpuC split-intein comprises the amino acid sequence of SEQ ID NO: 1754.

In some embodiments, the split inteins are M86N and M86C split inteins (alternatively referred elsewhere herein as M86-N2 and M86-C2 split inteins). In one embodiment, the M86N split intein comprises an amino acid sequence having at least 90% identity (e.g., at least 90%, 91%, 92%, 93%, 94%, 95%, 96%, 97%, 98%, 99%, or 100%) to SEQ ID NO: 1755 and the M86C split-intein comprises an amino acid sequence having at least 90% identity (e.g., at least 90%, 91%, 92%, 93%, 94%, 95%, 96%, 97%, 98%, 99%, or 100%) to SEQ ID NO: 1756. In one embodiment, the M86C split intein comprises the amino acid sequence of SEQ ID NO: 1755 and the M86N split-intein comprises the amino acid sequence of SEQ ID NO: 1756.

In some embodiments, the split inteins are NpuSspN and NpuSspC split inteins, (alternatively referred elsewhere herein as NpuSsp-N3 and NpuSsp-C3 split inteins), In some embodiments, the NpuSspN split intein comprises an amino acid sequence having at least 90% identity (e.g., at least 90%, 91%, 92%, 93%, 94%, 95%, 96%, 97%, 98%, 99%, or 100%) to SEQ ID NO: 1757 and the NpuSspC split-intein comprises an amino acid sequence having at least 90% identity (e.g., at least 90%, 91%, 92%, 93%, 94%, 95%, 96%, 97%, 98%, 99%, or 100%) to SEQ ID NO: 1758. In some embodiments, the NpuSspN split intein comprises the amino acid sequence of SEQ ID NO: 1757 and the NpuSspC split-intein comprises the amino acid sequence of SEQ ID NO: 1758.

In some embodiments, the split inteins are gp41-1-N and gp41-1-C split inteins (alternatively referred elsewhere herein as gp41-1-N2 and gp41-1-C2 split inteins, respectively). In some embodiments, the gp41-1-N split intein comprises an amino acid sequence having at least 90% identity (e.g., at least 90%, 91%, 92%, 93%, 94%, 95%, 96%, 97%, 98%, 99%, or 100%) to SEQ ID NO: 1759 and the gp41-1-C split-intein comprises an amino acid sequence having at least 90% identity (e.g., at least 90%, 91%, 92%, 93%, 94%, 95%, 96%, 97%, 98%, 99%, or 100%) to SEQ ID NO: 1760. In some embodiments, the gp41-1-N split intein comprises the amino acid sequence of SEQ ID NO: 1759 and the gp41-1-C split-intein comprises the amino acid sequence of SEQ ID NO: 1760.

In some embodiments, the split inteins are gp41-8-N and gp41-8-C split inteins (alternatively referred elsewhere herein as gp41-8-N2 and gp41-8-C2 split inteins, respectively). In some embodiments, the gp41-8-N split intein comprises an amino acid sequence having at least 90% identity (e.g., at least 90%, 91%, 92%, 93%, 94%, 95%, 96%, 97%, 98%, 99%, or 100%) to SEQ ID NO: 1761 and the gp41-8-C split-intein comprises an amino acid sequence having at least 90% identity (e.g., at least 90%, 91%, 92%, 93%, 94%, 95%, 96%, 97%, 98%, 99%, or 100%) to SEQ ID NO: 1762 In some embodiments, the gp41-8-N intein comprises the amino acid sequence of SEQ ID NO: 1761 and the gp41-8-C split-intein comprises the amino acid sequence of SEQ ID NO: 1762.

In some embodiments, the split inteins are NrdJ-1-N and NjdJ-1-C split inteins (alternatively referred elsewhere herein as NrdJ-1-N2 and NjdJ-1-C2 split inteins, respectively). In some embodiments, the NrdJ-1-N split intein comprises an amino acid sequence having at least 90% identity (e.g., at least 90%, 91%, 92%, 93%, 94%, 95%, 96%, 97%, 98%, 99%, or 100%) to SEQ ID NO: 1763 and the NjdJ-1-C split-intein comprises an amino acid sequence having at least 90% identity (e.g., at least 90%, 91%, 92%, 93%, 94%, 95%, 96%, 97%, 98%, 99%, or 100%) to SEQ ID NO: 1764. In some embodiments, the NrdJ-1-N intein comprises the amino acid sequence of SEQ ID NO: 1763 and the NjdJ-1-C split-intein comprises the amino acid sequence of SEQ ID NO: 1764.

In some embodiments, the split inteins are IMPDH-1-N and IMPDH-1-C split inteins (alternatively referred elsewhere herein as IMPDH-1-N2 and IMPDH-1-C2 split inteins, respectively). In some embodiments, the IMPDH-1-N split intein comprises an amino acid sequence having at least 90% identity (e.g., at least 90%, 91%, 92%, 93%, 94%, 95%, 96%, 97%, 98%, 99%, or 100%) to SEQ ID NO: 1765 and the IMPDH-1-C split-intein comprises an amino acid sequence having at least 90% identity (e.g., at least 90%, 91%, 92%, 93%, 94%, 95%, 96%, 97%, 98%, 99%, or 100%) to SEQ ID NO: 1766. In some embodiments, the IMPDH-1-N intein comprises the amino acid sequence of SEQ ID NO: 1765 and the IMPDH-1-C split-intein comprises the amino acid sequence of SEQ ID NO: 1766.

In some embodiments, the split inteins are SspDnaX-N and SspDnaX-C split inteins (alternatively referred elsewhere herein as SspDnaX-N2 and SspDnaX-C2 split inteins, respectively). In some embodiments, the SspDnaX-N split intein comprises an amino acid sequence having at least 90% identity (e.g., at least 90%, 91%, 92%, 93%, 94%, 95%, 96%, 97%, 98%, 99%, or 100%) to SEQ ID NO: 1767 and the SspDnaX-C split-intein comprises an amino acid sequence having at least 90% identity (e.g., at least 90%, 91%, 92%, 93%, 94%, 95%, 96%, 97%, 98%, 99%, or 100%) to SEQ ID NO: 1768. In some embodiments, the SspDnaX-N intein comprises the amino acid sequence of SEQ ID NO: 1767 and the SspDnaX-C split-intein comprises the amino acid sequence of SEQ ID NO: 1768.

In some embodiments, the split inteins are SspGyrB-N and SspGyrB-C split inteins (alternatively referred elsewhere herein as SspGyrB-N2 and SspGyrB-C2 split inteins, respectively). In some embodiments, the SspGyrB-N split intein comprises an amino acid sequence having at least 90% identity (e.g., at least 90%, 91%, 92%, 93%, 94%, 95%, 96%, 97%, 98%, 99%, or 100%) to SEQ ID NO: 1769 and the SspGyrB-C split-intein comprises an amino acid sequence having at least 90% identity (e.g., at least 90%, 91%, 92%, 93%, 94%, 95%, 96%, 97%, 98%, 99%, or 100%) to SEQ ID NO: 1770. In some embodiments, the SspGyrB-N intein comprises the amino acid sequence of SEQ ID NO: 1769 and the SspGyrB-C split-intein comprises the amino acid sequence of SEQ ID NO: 1770.

In some embodiments, the split inteins are Cth-Ter-N and Cth-Ter-C split inteins (alternatively referred elsewhere herein as Cth-Ter-N2 and Cth-Ter-C2 split inteins, respectively). In some embodiments, the Cth-Ter-N split intein comprises an amino acid sequence having at least 90% identity (e.g., at least 90%, 91%, 92%, 93%, 94%, 95%, 96%, 97%, 98%, 99%, or 100%) to SEQ ID NO: 1771 and the Cth-Ter-C split-intein comprises an amino acid sequence having at least 90% identity (e.g., at least 90%, 91%, 92%, 93%, 94%, 95%, 96%, 97%, 98%, 99%, or 100%) to SEQ ID NO: 1772. In some embodiments, the Cth-Ter-N intein comprises the amino acid sequence of SEQ ID NO: 1771 and the Cth-Ter-C split-intein comprises the amino acid sequence of SEQ ID NO: 1772.

In some embodiments, the split inteins are NrdA-2-N and NrdA-2-C split inteins (alternatively referred elsewhere herein as NrdA-2-N2 and NrdA-2-C2 split split inteins, respectively). In some embodiments, the NrdA-2-N split intein comprises an amino acid sequence having at least 90% identity (e.g., at least 90%, 91%, 92%, 93%, 94%, 95%, 96%, 97%, 98%, 99%, or 100%) to SEQ ID NO: 1773 and the NrdA-2-C split-intein comprises an amino acid sequence having at least 90% identity (e.g., at least 90%, 91%, 92%, 93%, 94%, 95%, 96%, 97%, 98%, 99%, or 100%) to SEQ ID NO: 1774. In some embodiments, the NrdA-2-N intein comprises the amino acid sequence of SEQ ID NO: 1773 and the NrdA-2-C split-intein comprises the amino acid sequence of SEQ ID NO: 1774.

In some embodiments, the split inteins are Mja-KlbA-N and Mja-KlbA-C split inteins (alternatively referred elsewhere herein as Mja-KlbA-N2 and Mja-KlbA-C2 split split inteins, respectively). In some embodiments, the Mja-KlbA-N split intein comprises an amino acid sequence having at least 90% identity (e.g., at least 90%, 91%, 92%, 93%, 94%, 95%, 96%, 97%, 98%, 99%, or 100%) to SEQ ID NO: 1775 and the Mja-KlbA-C split-intein comprises an amino acid sequence having at least 90% identity (e.g., at least 90%, 91%, 92%, 93%, 94%, 95%, 96%, 97%, 98%, 99%, or 100%) to SEQ ID NO: 1776. In some embodiments, the Mja-KlbA-N intein comprises the amino acid sequence of SEQ ID NO: 1775 and the Mja-KlbA-C split-intein comprises the amino acid sequence of SEQ ID NO: 1776.

In some embodiments, the split inteins are RBS1N and RBS1C split inteins (alternatively referred elsewhere herein as RBS1-N and RBS1-C split inteins, respectively). In some embodiments, the RBS1N split intein comprises an amino acid sequence having at least 90% identity (e.g., at least 90%, 91%, 92%, 93%, 94%, 95%, 96%, 97%, 98%, 99%, or 100%) to SEQ ID NO: 1777 and the RBS1C split-intein comprises an amino acid sequence having at least 90% identity (e.g., at least 90%, 91%, 92%, 93%, 94%, 95%, 96%, 97%, 98%, 99%, or 100%) to SEQ ID NO: 1778. In some embodiments, the RBS1N intein comprises the amino acid sequence of SEQ ID NO: 1777 and the RBS1C split-intein comprises the amino acid sequence of SEQ ID NO: 1778.

In some embodiments, the split inteins are TE3S1IN and TE3S11C split inteins (alternatively referred elsewhere herein as TE3S11-N and TE3S11-C split inteins, respectively). In some embodiments, the TE3S1IN split intein comprises an amino acid sequence having at least 90% identity (e.g., at least 90%, 91%, 92%, 93%, 94%, 95%, 96%, 97%, 98%, 99%, or 100%) to SEQ ID NO: 1779 and the TE3S11C split-intein comprises an amino acid sequence having at least 90% identity (e.g., at least 90%, 91%, 92%, 93%, 94%, 95%, 96%, 97%, 98%, 99%, or 100%) to SEQ ID NO: 1780. In some embodiments, the TE3S11N intein comprises the amino acid sequence of SEQ ID NO: 1779 and the TE3S11C split-intein comprises the amino acid sequence of SEQ ID NO: 1780.

In some embodiments, the split inteins are RmaN and RmaC split inteins (alternatively referred elsewhere herein as Rma-N and Rma-C split inteins, respectively). In some embodiments, the RmaN split intein comprises an amino acid sequence having at least 90% identity (e.g., at least 90%, 91%, 92%, 93%, 94%, 95%, 96%, 97%, 98%, 99%, or 100%) to SEQ ID NO: 1781 and the RmaC split-intein comprises an amino acid sequence having at least 90% identity (e.g., at least 90%, 91%, 92%, 93%, 94%, 95%, 96%, 97%, 98%, 99%, or 100%) to SEQ ID NO: 1782. In some embodiments, the RmaN intein comprises the amino acid sequence of SEQ ID NO: 1781 and the RmaC split-intein comprises the amino acid sequence of SEQ ID NO: 1782.

The split inteins may each be operably linked to flanking polypeptide sequences by a way of additional amino acids (e.g., a peptide linker). Accordingly, in some embodiments, the first polynucleotide further encodes a first peptide linker that operably links the first split intein to the SpyCas9 nickase. In some embodiments, the second polynucleotide further encodes a second peptide linker that operably links the second split intein to the DNA-dependent DNA polymerase. The peptide linker may include those known in the art or peptide linkers further described herein.

Additional amino acids can also result from the residual intein amino acid sequence remaining in the construct after intein splicing. For example, in some embodiments, the amino acid sequence flanking the CfaN split intein is AEY (referred to as “CfaN_flank” elsewhere herein); in other embodiments, in some embodiments, the amino acid sequence flanking the CfaN split intein is RGK. In some embodiments, the amino acid sequence flanking the CfaC split intein is CFN (referred to as “CfaC_flank” elsewhere herein). Accordingly, splicing of CfaN and CfaC split inteins can result in the amino acid sequence “AEYCFN” or “RGKCFN” at the splice site. See also Table 4C (disclosing the N-split intein and C-split intein constructs including these sequence flanking the split inteins). This residual intein sequence, or “intein scar,” generally does not impact the function of the fusion protein. Other intein scar sequences that may arise following intein fusion include, but are not limited to, the following amino acid sequences: GWNCML, GLTCMY, RGKCWE, GALCWN, AEKCWL, and GDLCKQ.

In some embodiments, the first and/or second polypeptide encoding the split inteins and the SpyCas9 nickase or DNA-dependent DNA polymerase may further encode additional elements described herein in the context of single polypeptide fusions (e.g., peptide linkers, a heterologous NLS, a chromatin remodeler (e.g., HMGB1), and/or an enhancer of template-based genome editing (e.g., VPX and/or FEN-1).

In some embodiments, the first and/or second polypeptide encoding the split inteins further comprises a heterologous nuclear localization signal (NLS). For example, in some embodiments, the first polynucleotide further encodes a heterologous NLS that is operably linked to the SpyCas9 nickase. In some embodiments, the second polynucleotide further encodes a heterologous NLS that is operably linked to the DNA-dependent DNA polymerase. In some embodiments, the heterologous nuclear localization signal is selected from an SV40 NLS, SV40-cMyc, and nucleoplasmin.

In some embodiments, the first and/or second polypeptide encoding the split inteins further comprises one or more enhancers of template-based genome editing (e.g., Vpx and/or FEN-1). In some embodiments, the first and/or second polypeptide encoding the split inteins may further comprise two or more enhancers of template-based genome editing (e.g., Vpx and FEN-1). For example, in some embodiments, Vpx and/or FEN1 are encoded on the first polynucleotide and operably linked to the SpyCas9 nickase. In some embodiments, Vpx and/or FEN-1 are encoded on the second polynucleotide and operably linked to the DNA-dependent DNA polymerase. Alternatively, the one or more enhancers of template-based genome editing (e.g., Vpx and/or FEN-1) are encoded on a third polynucleotide (i.e., independent of the polynucleotides encoding the split inteins).

In some embodiments, the first and/or second polypeptide encoding the split inteins further comprises a 5′ cap. In some embodiments, the 5′cap is selected from Cap 0, Cap 1, and Cap 2. In certain embodiments, the 5′ cap is a cap analog. Examples of RNA caps are known in the art and further described herein.

In some embodiments, the first polynucleotide encodes a fusion polypeptide comprising one or more NLSs, the SpyCas9 nickase, one or more peptide linkers, and the first split-intein. In some embodiments, the first polynucleotide encodes a fusion polypeptide comprising sequentially from N-terminus to C-terminus an NLS, the SpyCas9 nickase, and the second split-intein. In some embodiments, the first polynucleotide further comprises a peptide linker between the NLS and the SpyCas9 nickase. In some embodiments, the first polynucleotide further comprises a peptide linker between the SpyCas9 nickase and the second split-intein.

In some embodiments, the second polynucleotide encodes a fusion polypeptide comprising the second split-intein, the DNA-dependent DNA polymerase, one or more peptide linkers, and one or more NLSs. In some embodiments, the second polynucleotide encodes a fusion polypeptide comprising sequentially from N-terminus to C-terminus the second split-intein, the DNA-dependent DNA polymerase, and an NLS. In some embodiments, the second polynucleotide further comprises a peptide linker between the second split intein and the DNA-dependent DNA polymerase. In some embodiments, the second polynucleotide further comprises a peptide linker between the NLS and the DNA-dependent DNA polymerase.

In some embodiments, the first and/or second polypeptide encoding the split inteins further comprises one or more chromatin remodelers (e.g., HMGB1). In some embodiments, the at least one chromatin remodeler comprises an HMGB1 polypeptide. In some embodiments, the HMGB1 polypeptide comprises at least 90% sequence identity (e.g., at least 90%, 91%, 92%, 93%, 94%, 95%, 96%, 97%, 98%, 99%, or 100%) to any one of SEQ ID NOs: 1784-1787 and 1789-1792 or that is encoded by a nucleic acid having at least 80%, 85%, 90%, 91%, 92%, 93%, 94%, 95%, 96%, 97%, 98%, or 99% identity to any one of SEQ ID NOs: 1794-1802. In some embodiments, the HMGB1 polypeptide comprises the amino acid sequence of any one of SEQ ID NOs: 1784-1787 and 1789-1792 or is encoded by a nucleic acid comprising any one of SEQ ID NOs: 1794-1802. In some embodiments, the HMGB1 polypeptide comprises at least 80%, 85%, 90%, 91%, 92%, 93%, 94%, 95%, 96%, 97%, 98%, or 99% identity to SEQ ID NO: 1789 or that is encoded by a nucleic acid having at least 80%, 85%, 90%, 91%, 92%, 93%, 94%, 95%, 96%, 97%, 98%, or 99% identity to SEQ ID NO: 1799. In some embodiments, the first polynucleotide encodes one or more chromatin remodeler operably linked to the SpyCas9 nickase. In some embodiments, the second polynucleotide encodes one or more chromatin remodeler operably linked to the DNA-dependent DNA polymerase.

Further provided herein is a method of generating a single polypeptide fusion comprising a SpyCas9 nickase and a DNA-dependent DNA polymerase, wherein the method comprises expressing the first and second polynucleotides comprising the split inteins described herein in a cell to form a polypeptide fusion comprising a DNA-dependent DNA polymerase and a SpyCas9 nickase (nCas9) via intein-mediated protein splicing. The resulting single polypeptide fusion may be used in combination with a template guide RNA in accordance with any of the systems and methods described herein for templated gene editing.

In some embodiments, the single polypeptide (i.e., comprising both the SpyCas9 nickase and the DNA-dependent DNA polymerase) further comprises a heterologous nuclear localization signal (NLS). In some embodiments, the single polypeptide comprises a heterologous NLS comprising a c-Myc, SV40, nucleoplasmin, or snurportin-1 importin-β NLS. In some embodiments, the single polypeptide comprises a heterologous NLS comprising a c-Myc NLS. In some embodiments, the single polypeptide comprises a heterologous NLS comprising an SV40 NLS. In some embodiments, the single polypeptide comprises a heterologous NLS comprising a nucleoplasmin NLS. In some embodiments, the polypeptide comprises a C-terminal heterologous NLS. In some embodiments, the polypeptide comprises an N-terminal heterologous NLS. In some embodiments, the heterologous NLS is between the SpyCas9 nickase and the DNA-dependent DNA polymerase wherein the nickase and polymerase are in a single fusion protein. In some embodiments, the single polypeptide comprises two heterologous NLSs. In some embodiments, the single polypeptide comprises a C-terminal heterologous NLS and an N-terminal heterologous NLS. In some embodiments, the single polypeptide comprises an NLS between the SpyCas9 nickase and the DNA-dependent DNA polymerase and an NLS at the N-terminus or at the C-terminus of the polypeptide. In some embodiments, the first and second heterologous NLS are the same. In some embodiments, the first and second heterologous NLS are different.

In some embodiments, the SpyCas9 nickase is N-terminal of the DNA-dependent DNA polymerase. In some embodiments, the SpyCas9 nickase is C-terminal of the DNA-dependent DNA polymerase.

In some embodiments, the single polypeptide (i.e., comprising both the SpyCas9 nickase and the DNA-dependent DNA polymerase) further comprises one or more linkers. For example, the single polypeptide may include one or more peptide linkers between elements encoded by different open reading frames. In some embodiments, SpyCas9 nickase and DNA-dependent DNA polymerase are joined by a peptide linker. In some embodiments, the linker is an organic molecule, polymer, or chemical moiety. In some embodiments, the linker is a peptide linker. In some embodiments, the nucleic acid encoding the polypeptide comprising the SpyCas9 nickase or the DNA-dependent DNA polymerase further comprises a sequence encoding the peptide linker. In some embodiments, one or more NLS are joined to the SpyCas9 nickase or the DNA dependent DNA polymerase by a linker, e.g., a peptide linker.

In some embodiments, the peptide linker is any stretch of amino acids having at least 1, 2, 3, 4, 5, 6, 7, 8, 9, 10, 15, 20, 25, 30, 40, 50, 60, 70, or 80 amino acids.

In some embodiments, the peptide linker is any stretch of amino acids having at least 11, 21, 31, 41, 51, 61, 71, 81, or 91 amino acids. In some embodiments, the peptide linker comprises 4, 5, 6, 7, 8, 9, 10, 11, 12, 13, 14, 15, 16, 17, 18, 19, 20, 21, 22, 23, 24, 25, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, the peptide linker is 4, 5, 6, 7, 8, 9, 10, 11, 12, 13, 14, 15, 16, 17, 18, 19, 20, 21, 22, 23, 24, 25, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids in length. In some embodiments, the peptide linker is 4 amino acids in length. In some embodiments, the peptide linker is 30 amino acids in length. In some embodiments, the peptide linker is 33 amino acids.

In some embodiments, the peptide linker comprises an amino acid sequence that is at least 90%, 92%, 93%, 94%, 95%, 97%, 99%, or 100% identical to any one of SEQ ID NO: 1596-1603, 1604-1665, 1735, 1738, 1815, 1825, 1827, and 1829-1830, or that is encoded by a nucleic acid sequence that is at least 90%, 91%, 92%, 93%, 94%, 95%, 96%, 97%, 98%, 99%, or 100% identical to any one of SEQ ID NOs: 1730-1734, 1736, 1737, 1739, 1810-1814, and 1816. In some embodiments, the peptide linker comprises an amino acid sequence of any one of SEQ ID NO: 1596-1603, 1604-1665, 1735, 1738, 1815, 1825, 1827, and 1829-1830 or that is encoded by a nucleic acid sequence that is at least 90%, 91%, 92%, 93%, 94%, 95%, 96%, 97%, 98%, 99%, or 100% identical to any one of SEQ ID NOs: 1730-1734, 1736, 1737, 1739, 1810-1814, and 1816.

In some embodiments, the peptide linker comprises 14 amino acid acids (e.g., Linker 14; L14; SEQ ID NO: 1596). In some embodiments, the peptide linker comprises 16 amino acids (e.g., Linker 16; L16; SEQ ID NO: 1597). In some embodiments, the peptide linker comprises 20 amino acid acids (e.g., Linker 20; L20; MGKRTADGSEFESPKKKRKV (SEQ ID NO: 1827)). In some embodiments, the peptide linker comprises 30 amino acid acids (e.g., Linker 30; L30; SGGSKRTADGSEFESPKKKRKVSGGSSGGS (SEQ ID NO: 1829)). In some embodiments, the peptide linker comprises 41 amino acid acids (e.g., Linker 41; L41; GAPESATESGGTSTESEGSAGTSTESEGSAGSAGSTSGSSS (SEQ ID NO: 1735)). In some embodiments, the peptide linker comprises 42 amino acid acids (e.g., Linker 42; L42; SEQ ID NO: 1600). In some embodiments, the peptide linker comprises 59 amino acid acids (e.g., Linker 59; L59; SEQ ID NO: 1830). In some embodiments, the peptide linker comprises GH5 (SEQ ID: 1661). In some embodiments, the linker comprises G4S (GGGGS (SEQ ID NO: 1605)).

In some embodiments, the fusion protein comprises a peptide linker between the SpyCas9 nickase and the DNA-dependent DNA polymerase and the peptide linker comprises an amino acid sequence of any one of SEQ ID NOs: 1596-1600; or an amino acid sequence is at least 85%, 90%, or 95% identical to any one of SEQ ID NOs: 1596-1600.

Other types of peptide linkers may be used herein. The peptide linker may be the 16 residue “XTEN” linker, or a variant thereof (See, e.g., Schellenberger et al. A recombinant polypeptide extends the in vivo half-life of peptides and proteins in a tunable manner. Nat. Biotechnol. 27, 1186-1190 (2009)). In some embodiments, the XTEN linker comprises a sequence that is any one of SGSETPGTSESATPES (SEQ ID NO: 1601), SGSETPGTSESA (SEQ ID NO: 1602), or SGSETPGTSESATPEGGSGGS (SEQ ID NO: 1603). In some embodiments, the XTEN linker consists of the sequence SGSETPGTSESATPES (SEQ ID NO: 1601), SGSETPGTSESA (SEQ ID NO: 1602), or SGSETPGTSESATPEGGSGGS (SEQ ID NO: 1603).

In some embodiments, the peptide linker comprises a (GGGGS)ₙ (e.g., SEQ ID NOs: 1605, 1609-1611, 1614-1618, 1620-1631, or 1633-1659), a (G)ₙ, an (EAAAK), (e.g., SEQ ID NOs: 1606, 1610-1612, 1615-1618, 1620-1631, or 1634-1660), a (GGS)ₙ, an SGSETPGTSESATPES (SEQ ID NO: 1601) motif (see, e.g., Guilinger J P, Thompson D B, Liu D R. Fusion of catalytically inactive Cas9 to FokI nuclease improves the specificity of genome modification. Nat. Biotechnol. 2014; 32 (6): 577-82; the entire contents are incorporated herein by reference), or an (XP)ₙ motif, or a combination of any of these, wherein n is independently an integer between 1 and 30. See, WO2015089406, e.g., paragraph [0012], the entire content of which is incorporated herein by reference.

In some embodiments, the peptide linker comprises a sequence selected from SEQ ID NOs: 1596-1665, 1735, 1738, 1815, 1825, 1827, and 1829-1830. In some embodiments, the peptide linker comprises a sequence selected from SEQ ID NOs: 1596-1600. In some embodiments, the peptide linker comprises a sequence selected from SEQ ID NOs: 1735, 1738, 1815, 1825, 1827, and 1829-1830. In some embodiments, the peptide linker comprises SEQ ID NO: 1829. In some embodiments, the peptide linker comprises SEQ ID NO: 1738.

In some embodiments, wherein the composition comprises (a) a single polypeptide comprising a SpyCas9 nickase and a DNA-dependent DNA polymerase, the single polypeptide is at least 90%, 91%, 92%, 93%, 94%, 95%, 96%, 97%, 98%, 99%, or 100% identical to any one of SEQ ID NOs: 1024, 1027, 1030, 1033, 1036, 1039, 1042, 1045, 1048, 1053, 1056, 1059, 1074, 1081, 1084, 1086, 1094, 1097, 1102, 1114, 1124, 1129, 1134, 1137, 1142, 1147, 1152, 1157, 1162, 1167, 1172, 1192, 1195, 1198, 1219, 1224, 1229, 1234, 1242, 1244, 1247, 1249, 1252, 1254, 1257, 1259, and 1268, and 1273, 1276, 1300, 1303, 1307, 1373, 1376, 1388, 1447, 1450, 1453, 1923, 1926, 1929, 1932, 1935, and 1938. In some embodiments, the single polypeptide is at least 90%, 91%, 92%, 93%, 94%, 95%, 96%, 97%, 98%, 99%, or 100% identical to SEQ ID NO: 1219. In some embodiments, the single polypeptide comprises SEQ ID NO: 1219. In some embodiments, the single polypeptide is at least 90%, 91%, 92%, 93%, 94%, 95%, 96%, 97%, 98%, 99%, or 100% identical to SEQ ID NO: 1234. In some embodiments, the single polypeptide comprises SEQ ID NO: 1234. In some embodiments, the single polypeptide is encoded by a nucleotide sequence that has at least 90%, 91%, 92%, 93%, 94%, 95%, 96%, 97%, 98%, 99%, or 100% identity to any one of SEQ ID NOs: 1022, 1023, 1025, 1026, 1028, 1029, 1031, 1032, 1034, 1035, 1037, 1038, 1040, 1041, 1043, 1044, 1046, 1047, 1052, 1054, 1055, 1057, 1058, 1073, 1085, 1092, 1093, 1095, 1096, 1100, 1101, 1113, 1122, 1123, 1128, 1133, 1135, 1136, 1141, 1146, 1151, 1156, 1161, 1166, 1171, 1190, 1191, 1193, 1194, 1196, 1197, 1217, 1218, 1228, 1233, 1240, 1243, 1245, 1248, 1250, 1253, 1255, 1258, and 1267. In some embodiments, the single polypeptide is encoded by a nucleotide sequence that has at least 90%, 91%, 92%, 93%, 94%, 95%, 96%, 97%, 98%, 99%, or 100% identical to SEQ ID NO: 1218. In some embodiments, the single polypeptide is encoded by a nucleic acid comprising an open reading frame (ORF) comprising a sequence that has at least 90%, 91%, 92%, 93%, 94%, 95%, 96%, 97%, 98%, 99%, or 100% identical to SEQ ID NO: 1218. In some embodiments, the single polypeptide is encoded by a nucleic acid comprising an open reading frame (ORF) comprising the sequence of SEQ ID NO: 1218. In some embodiments, the single polypeptide is encoded by a nucleotide sequence that has at least 90%, 91%, 92%, 93%, 94%, 95%, 96%, 97%, 98%, 99%, or 100% identical to SEQ ID NO: 1232 or 1233. In some embodiments, the single polypeptide is encoded by a nucleic acid comprising an open reading frame (ORF) comprising a sequence that has at least 90%, 91%, 92%, 93%, 94%, 95%, 96%, 97%, 98%, 99%, or 100% identical to SEQ ID NO: 1233. In some embodiments, the single polypeptide is encoded by a nucleic acid comprising an open reading frame (ORF) comprising the sequence of SEQ ID NO: 1233. It is to be understood that, if the sequence includes a HiBiT tag, the tag (and preceding linker, if present) is not included in the amino acid sequence within the scope of the claimed invention.

In some embodiments, wherein the SpyCas9 nickase and DNA-dependent DNA polymerase are expressed from separate ORFs, a first polypeptide comprising the SpyCas9 nickase portion thereof, and a first split intein; and a second polypeptide comprising the DNA-dependent DNA polymerase portion thereof, and a second split intein, wherein (1) the first polypeptide comprises at least 90%, 91%, 92%, 93%, 94%, 95%, 96%, 97%, 98%, 99%, or 100% identical to any one of SEQ ID NOs: 1279, 1282, 1364, 1367, and 1379, 1392, 1396, 1400, 1404, 1408, 1412, 1416, 1420, 1424, 1428, 1432, 1436, 1440, 1444, 1908, 1914, 1941, and 1944; or is encoded by a nucleic acid comprising an ORF comprising a nucleotide sequence at least 90%, 91%, 92%, 93%, 94%, 95%, 96%, 97%, 98%, 99%, or 100% identical to any one of SEQ ID NOs: 1278, 1281, 1363, 1366, and 1378; 1391, 1395, 1399, 1403, 1407, 1411, 1415, 1419, 1423, 1427, 1431, 1435, 1439, 1443, 1907, 1913, 1940, and 1943; or is encoded by a sequence at least 90%, 91%, 92%, 93%, 94%, 95%, 96%, 97%, 98%, 99%, or 100% identical to any one of SEQ ID NOs: 1277, 1280, 1362, 1365, 1377, 1906, 1912, 1939, 1942, 3006-3008, 3010, 3012, and 3017-3018, or any other sequences encoding the first polypeptide shown in Table 6; and (2) the second polypeptide comprises at least 90%, 91%, 92%, 93%, 94%, 95%, 96%, 97%, 98%, 99%, or 100% identical to any one of SEQ ID NOs: 1285, 1288, 1291, 1294, 1297, 1370, 1382, 1385, 1390, 1394, 1398, 1402, 1406, 1410, 1414, 1422, 1426, 1430, 1434, 1438, 1442, 1902, 1905, 1911, 1917, 1920, 1947, 1950, 1953, 1956, 1959, or is encoded by a nucleic acid comprising an ORF comprising a nucleotide sequence at least 90%, 91%, 92%, 93%, 94%, 95%, 96%, 97%, 98%, 99%, or 100% identical to any one of SEQ ID NOs: 1284, 1287, 1290, 1293, 1296, 1369, 1381, 1384, 1389, 1393; 1397, 1401, 1405, 1409, 1413, 1417, 1421, 1425, 1429, 1433, 1437, 1441, 1901, 1904, 1910, 1916, 1918, 1946, 1949, 1952, 1955, 1958; or is encoded by a sequence at least 90%, 91%, 92%, 93%, 94%, 95%, 96%, 97%, 98%, 99%, or 100% identical to any one of SEQ ID NOs: 1283, 1286, 1289, 1292, 1295, 1368, 1380, 1383, 1900, 1903, 1909, 1915, 1919, 1945, 1948, 1951, 1954, 1957, 3001-3005, 3009, 3011, 3013-3016, and 3019-3022 or any other sequences encoding the second polypeptide shown in Table 6.

In some embodiments, the present disclosure provides for (a) a first polynucleotide comprising an ORF encoding a polypeptide comprising the SpyCas9 nickase or portion thereof, and a first split intein and (b) a second polynucleotide comprising an ORF encoding a polypeptide comprising the DNA-dependent DNA polymerase, or portion thereof, and a second split intein, wherein the first polynucleotide comprises an ORF comprising a nucleotide sequence at least 90%, 91%, 92%, 93%, 94%, 95%, 96%, 97%, 98%, 99%, or 100% identical to any one of SEQ ID NOs: 1278, 1281, 1284, 1287, 1290, 1363, 1366, and 1378; and (2) the second polynucleotide comprises an ORF comprising a nucleotide sequence at least 90%, 91%, 92%, 93%, 94%, 95%, 96%, 97%, 98%, 99%, or 100% identical to any one of SEQ ID NOs: 1293, 1296, 1369, 1381, and 1384.

In some embodiments, the present disclosure provides for a first mRNA comprising an ORF encoding a polypeptide comprising the SpyCas9 nickase or portion thereof and an N-intein, the first mRNA comprising a nucleotide sequence having at least 85%, 86%, 87%, 88%, 89%, 90%, 91%, 92%, 93%, 94%, 95%, 96%, 97%, 98%, 99%, or 100% identity to any one of SEQ ID NOs: 1277, 1280, 1283, 1286, 1289, 1362, 1365, 1377, 3006-3008, 3010, 3012, 3017, and 3018; or a second mRNA comprising an ORF encoding a polypeptide comprising the DNA-dependent DNA polymerase, or portion thereof, and a C split intein, the second mRNA comprising a nucleotide sequence having at least 85%, 86%, 87%, 88%, 89%, 90%, 91%, 92%, 93%, 94%, 95%, 96%, 97%, 98%, 99%, or 100% identity to any one of SEQ ID NOs: 1292, 1295, 1368, 1380, 1383, 3001-3005, 3009, 3011, 3013-3016, 3019-3022.

### The SpyCas9 Nickase and DNA-Dependent DNA Polymerase May not be Covalently Linked

In some embodiment, the SpyCas9 nickase and DNA-dependent DNA polymerase are provided in separate polypeptides. In some embodiments, the SpyCas9 nickase and the DNA-dependent DNA polymerase are expressed from separate ORFs. In some embodiments, the SpyCas9 nickase and the DNA-dependent DNA polymerase are not covalently linked. In some embodiments, the SpyCas9 nickase and DNA-dependent DNA polymerase directly interact through non-covalent interactions. In some embodiments, the SpyCas9 nickase and the DNA-dependent DNA polymerase do not directly interact. In some embodiments, the SpyCas9 nickase and the DNA-dependent DNA polymerase both bind to a single biomolecule such as a template guide RNA. In some embodiments, the SpyCas9 nickase and the DNA-dependent DNA polymerase both bind to a template guide RNA. In some embodiments, wherein the SpyCas9 nickase and the DNA-dependent DNA polymerase both bind to a template guide RNA, the SpyCas9 nickase and the DNA-dependent DNA polymerase are operably linked by the guide RNA, the 3′ extension, and optionally, the affinity tag.

In some embodiments, the SpyCas9 nickase and DNA-dependent DNA polymerase directly interact through non-covalent interactions. In some embodiments, a first polypeptide comprising the SpyCas9 nickase and a second polypeptide comprising the DNA-dependent DNA polymerase each comprise, for example, a heterodimerization domain that mediates the binding of the first polypeptide to the second polypeptide. In some embodiments, the heterodimerization domains comprise: leucine zipper domains; PDZ domains; streptavidin and a streptavidin-binding domain; foldon domains; hydrophobic polypeptides; or an antibody or one or more binding fragments thereof (e.g., scFv, VHH domain) on one component that binds an epitope, either naturally occurring or inserted, in the other component. In some embodiments, the position of the heterodimer domain in the first and second polypeptides is independently selected.

In some embodiments, the SpyCas9 nickase and DNA-dependent DNA polymerase do not directly interact. As a first example, in some embodiments, the SpyCas9 nickase and DNA-dependent DNA polymerase may both non-covalently bind a single polynucleotide such as a template guide RNA. As a second example, in some embodiments, the SpyCas9 nickase may bind a first polynucleotide comprising a guide RNA and the DNA-dependent DNA polymerase may bind a second polynucleotide comprising a separable 3′ extension, wherein the first and second polynucleotides target the SpyCas9 nickase and DNA-dependent DNA polymerase to proximal genomic loci.

In some embodiments, wherein the SpyCas9 nickase and DNA-dependent DNA polymerase are provided in separate polypeptides and the SpyCas9 nickase or the DNA-dependent DNA polymerase comprises a heterologous NLS. In some embodiments, the SpyCas9 nickase comprises a heterologous NLS. In some embodiments the DNA-dependent DNA polymerase comprises a heterologous NLS. In some embodiments, the heterologous NLS of the SpyCas9 nickase or the DNA-dependent DNA polymerase comprises a c-Myc, SV40, nucleoplasmin, or snurportin-1 importin-β NLS. In some embodiments, the heterologous NLS of the SpyCas9 nickase or the DNA-dependent DNA polymerase comprises a c-Myc NLS. In some embodiments, the heterologous NLS of the SpyCas9 nickase or the DNA-dependent DNA polymerase comprises an SV40 NLS. In some embodiments, the heterologous NLS of the SpyCas9 nickase or the DNA-dependent DNA polymerase comprises a nucleoplasmin NLS. In some embodiments, the heterologous NLS of the SpyCas9 nickase or the DNA-dependent DNA polymerase comprises a C-terminal NLS. In some embodiments, the heterologous NLS comprises an N-terminal NLS. In some embodiments, the single polypeptide comprises two heterologous NLSs. In some embodiments, the single polypeptide comprises a first heterologous NLS at the N-terminus and a second heterologous NLS at the C-terminus. In some embodiments, the first and second heterologous NLS are the same. In some embodiments, the first and second heterologous NLS are different.

In some embodiments, the composition comprises (a) a first polypeptide comprising a SpyCas9 nickase and (b) a second polypeptide comprising a DNA-dependent DNA polymerase. In some embodiments, the first polypeptide is at least 90%, 91%, 92%, 93%, 94%, 95%, 96%, 97%, 98%, 99%, or 100% identical to SEQ ID NO: any one of SEQ ID NOs: 972, 974, 976, 982, 984, 986, 988, 990, 992, 994, 996, 998, 1000, and 1014; and the second polypeptide is at least 90%, 91%, 92%, 93%, 94%, 95%, 96%, 97%, 98%, 99%, or 100% identical to any one of SEQ ID NOs: 1064, 1069, 1177, or 1182. In some embodiments, the first polypeptide is encoded by a nucleotide sequence that has at least 90%, 91%, 92%, 93%, 94%, 95%, 96%, 97%, 98%, 99%, or 100% identity to any one of SEQ ID NOs: 971, 973, 975, 981, 983, 985, 987, 989, 991, 993, 995, 997, 999, 1010 and 1013; and the second polypeptide is encoded by a nucleotide sequence that has at least 90%, 91%, 92%, 93%, 94%, 95%, 96%, 97%, 98%, 99%, or 100% identity to any one of SEQ ID NOs: 1063, 1068, 1176, 1181.

### Additional Features

The following section provides additional features of the SpyCas9 nickase, DNA-dependent DNA polymerase, fusion protein, nucleic acid or nucleic acids encoding the same, guide RNAs, and compositions disclosed herein. In any of the embodiments set forth herein, the nucleic acid or nucleic acids may be one or more expression constructs comprising a promoter operably linked to an ORF encoding the SpyCas9 nickase, DNA-dependent DNA polymerase, or fusion proteins disclosed herein.

### Codon Optimization

In some embodiments, the nucleic acid or nucleic acids encoding a polypeptide or polypeptides comprising the SpyCas9 nickase, the DNA-dependent DNA polymerase, or fusion protein disclosed herein comprise one or more ORFs comprising one or more codon optimized nucleic acid sequences. See, e.g., WO2019/067910 A1 and WO2020/198641, both of which are incorporated herein by reference.

### Heterologous Functional Domains; Nuclear Localization Signals (NLSs)

In some embodiments, the heterologous functional domain may be capable of modifying the intracellular half-life of the SpyCas9 nickase, DNA-dependent DNA polymerase, or fusion protein disclosed herein. In some embodiments, the half-life of the SpyCas9 nickase, DNA-dependent DNA polymerase, or fusion protein disclosed herein may be increased. In some embodiments, the half-life of the SpyCas9 nickase, DNA-dependent DNA polymerase, or fusion protein disclosed herein may be reduced. In some embodiments, the heterologous functional domain may be capable of increasing the stability of the SpyCas9 nickase, DNA-dependent DNA polymerase, or fusion protein disclosed herein. In some embodiments, the heterologous functional domain may be capable of reducing the stability of the SpyCas9 nickase, DNA-dependent DNA polymerase, or fusion protein disclosed herein. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the polypeptide may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub1 in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Any known fluorescent proteins may be used as the marker domain such as GFP, YFP, EBFP, ECFP, DsRed or any other suitable fluorescent protein. In some embodiments, the marker domain may be a purification tag or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6xHis, 8xHis, biotin carboxyl carrier protein (BCCP), poly-His, and calmodulin. In some embodiments, the marker domain may be a reporter gene. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the SpyCas9 nickase, DNA-dependent DNA polymerase, or fusion protein disclosed herein to a specific organelle, cell type, tissue, or organ. In some embodiments, the heterologous functional domain may target the SpyCas9 nickase, DNA-dependent DNA polymerase, or fusion protein disclosed herein to mitochondria.

In some embodiments, the heterologous functional domain may facilitate transport of one or both of the SpyCas9 nickase and the DNA-dependent DNA polymerase proteins disclosed herein into the nucleus of a cell. For example, the heterologous functional domain may be a nuclear localization signal (NLS). In some embodiments, the fusion protein comprises a nuclear localization signal (NLS).

In some embodiments, the proteins disclosed herein may be fused with 1-5 NLS(s). In some embodiments, the fusion protein disclosed herein may be fused with two NLSs.

Where one NLS is used, the NLS may be fused at the N-terminus or the C-terminus of the fusion protein. In some embodiments, the fusion protein disclosed herein may be fused C-terminally to at least one NLS. An NLS may also be inserted within the fusion protein, e.g., between the SpyCas9 nickase and the DNA dependent DNA polymerase. In other embodiments, the fusion protein may be fused with more than one NLS.

In some embodiments, the fusion protein may be fused with 2, 3, 4, or 5 NLSs. In some embodiments, the fusion protein may be fused with two NLSs. In certain circumstances, the two NLSs may be the same (e.g., two SV40 NLSs) or different. For example, in some embodiments, the fusion protein may comprise an SV40 NLS and a C-Myc NLS. In some embodiments, the fusion protein is fused to two SV40 NLS sequences at the C-terminus. In some embodiments, the fusion protein may be fused with two NLSs, one at the N-terminus and one at the C-terminus. In some embodiments, the fusion protein may be fused with 3 NLSs.

In some embodiments, the fusion protein may be fused with no NLS.

In some embodiments, the NLS may be a monopartite sequence, such as, e.g., the SV40 NLS, for example, PKKKRKVE (SEQ ID NO: 1666), KKKRKVE (SEQ ID NO: 1667), PKKKRKV (SEQ ID NO: 1671) or PKKKRRV (SEQ ID NO: 1705). In some embodiments, wherein the NLS is a monopartite sequence, the NLS may be a c-myc like NLS, for example, PAAKKKKLD (SEQ ID NO: 1669). In some embodiments, the NLS may be a bipartite sequence, such as the NLS of nucleoplasmin, KRPAATKKAGQAKKKK (SEQ ID NO: 1706). One or more linkers are optionally included at the fusion site (e.g., between fusion protein disclosed herein and NLS).

In some embodiments, one or more NLS(s) according to any of the foregoing embodiments are present in the fusion protein in combination with one or more additional heterologous functional domains, such as any of the heterologous functional domains described below.

In some embodiments, the fusion protein comprises a nuclear localization signal (NLS) and the NLS is present at the C-terminus of the fusion protein. In some embodiments, the fusion protein comprises a nuclear localization signal (NLS) and the NLS is present at the N-terminus of the fusion protein. In some embodiments, the fusion protein comprises a nuclear localization signal (NLS) and the NLS is present at both the N-terminus and C-terminus of the fusion protein. In some embodiments, the fusion protein comprises a nuclear localization signal (NLS) and the NLS is present between the SpyCas9 protein and DNA dependent DNA polymerase. In some embodiments, the fusion protein comprises a nuclear localization signal (NLS) and the NLS is present within the linker sequence.

In some embodiments, the fusion protein comprises a nuclear localization signal (NLS), and wherein the NLS comprises a sequence having at least 80%, 85%, 90%, or 95% identity to any one of SEQ ID NOs: 1666-1671 and 1694-1706. In some embodiments, the fusion protein comprises one, two, or three nuclear localization signals (NLSs) independently selected from SEQ ID NOs: 1666-1671 and 1694-1706.

In some embodiments, the fusion protein comprises a nuclear localization signal (NLS), and wherein the NLS is encoded by a sequence having at least 80%, 85%, 90%, or 100% identity to the sequence of any one of SEQ ID NOs: 1666-1671 and 1694-1706.

### Chromatin Remodelers

Chromatin structure may impact the efficacy of various genome editing techniques, and target sequences located in chromatin-dense regions, in particular, may be less amenable to genome editing. For example, the CRISPR-associated nuclease Cas9 can exhibit insufficient editing of certain targets whose protospacer adjacent motifs (PAMs) are located within nucleosomes. Chromatin modulating peptides fused with Cas9 nucleases have been reported to improve editing efficiency at refractory target sites. Accordingly, in some embodiments, the system or fusion protein disclosed herein further comprises at least one chromatin remodeler. In some embodiments, the at least one chromatin remodeler is operably linked to the DNA-dependent DNA polymerase or SpyCas9 nickase (nCas9). In some embodiments, the at least one chromatin remodeler is operably linked to the DNA-dependent DNA polymerase. In some embodiments, the at least one chromatin remodeler is operably linked to the nCas9.

In some embodiments, the at least one chromatin remodeler is selected from a polypeptide comprising HMGB1, HMGN1, HMGN2, HMGN3a, HMGN3b, HMGN4, HMGN5, histone H1.0, histone H1.2, CHD1, ISWI, DNA helicase, TOP1, herpesvirus 8 LANA, CMV IE1 or a domain thereof.

In some embodiments, the at least one chromatin remodeler comprises HMGB1. In some embodiments, the at least one chromatin remodeler comprises an HMGB1 polypeptide. In some embodiments, the HMGB1 polypeptide comprises a sequence having at least 80%, 85%, 90%, 91%, 92%, 93%, 94%, 95%, 96%, 97%, 98%, or 99% identity to SEQ ID NO: 1783. In some embodiments, the HMGB1 polypeptide comprises the amino acid sequence of SEQ ID NO: 1783. In some embodiments, the HMGB1 polypeptide comprises a sequence having at least 80%, 85%, 90%, 91%, 92%, 93%, 94%, 95%, 96%, 97%, 98%, or 99% identity to any one of SEQ ID NOs: 1783-1792 and 1803-1804. In some embodiments, the HMGB1 polypeptide comprises the amino acid sequence of any one of SEQ ID NOs: 1783-1792 and 1803-1804. In some embodiments, the HMGB1 polypeptide comprises at least 80%, 85%, 90%, 91%, 92%, 93%, 94%, 95%, 96%, 97%, 98%, or 99% identity to SEQ ID NO: 1789 or that is encoded by a nucleic acid having at least at least 80%, 85%, 90%, 91%, 92%, 93%, 94%, 95%, 96%, 97%, 98%, or 99% identity to SEQ ID NO: 1799.

In some embodiments, the nCas9 and the DNA-dependent DNA polymerase are covalently linked sequentially in a polypeptide comprising from N-terminus to C-terminus a chromatin remodeler, the nCas9, and the DNA-dependent DNA polymerase. In some embodiments, the nCas9 and the DNA-dependent DNA polymerase are covalently linked sequentially in a polypeptide comprising from N-terminus to C-terminus the nCas9, a chromatin remodeler, and the DNA-dependent DNA polymerase. In some embodiments, the nCas9 and the DNA-dependent DNA polymerase are covalently linked sequentially in a polypeptide comprising from N-terminus to C-terminus a nCas9, the DNA-dependent DNA polymerase, and a chromatin remodeler.

In some embodiments, the nCas9 and the DNA-dependent DNA polymerase are covalently linked sequentially in a polypeptide from N-terminus to C-terminus, wherein the order is nuclear localization signal-chromatin remodeler-nCas9-DNA Dependent DNA polymerase-nuclear localization signal. In some embodiments, the nCas9 and the DNA-dependent DNA polymerase are covalently linked sequentially in a polypeptide from N-terminus to C-terminus, wherein the order is nuclear localization signal-nCas9-chromatin remodeler-DNA Dependent DNA polymerase-nuclear localization signal. In some embodiments, the nCas9 and the DNA-dependent DNA polymerase are covalently linked sequentially in a polypeptide from N-terminus to C-terminus, wherein the order is nuclear localization signal-nCas9-DNA Dependent DNA polymerase-chromatin remodeler-nuclear localization signal. In some embodiments, the polypeptide fusion further comprises one or more linkers between the nuclear localization signal(s), nCas9, DNA-dependent DNA polymerase, and/or the chromatin remodeler.

### HMGB1

The wildtype HMGB1 protein is a non-sequence-specific DNA-binding protein that interacts with DNA through its DNA-binding domains. HMGB1 binding to DNA introduces local DNA distortions, which may affect the structure and stability of nearby DNA-protein complexes. In particular, HMGB1 binding within the linker region of nucleosomal DNA may destabilize the nucleosome structure, thereby increasing the accessibility of the surrounding chromatin. See, e.g., Starkova T Y, Polyanichko A M, Artamonova T O, Tsimokha A S, Tomilin A N, Chikhirzhina E V. Structural Characteristics of High-Mobility Group Proteins HMGB1 and HMGB2 and Their Interaction with DNA. Int J Mol Sci. 2023 Feb. 10; 24(4):3577 and Lange S S, Vasquez K M. HMGB1: the jack-of-all-trades protein is a master DNA repair mechanic. Mol Carcinog. 2009 July; 48(7):571-80.

The wildtype HMGB1 protein comprises, from N-terminus to C-terminus, a Box A DNA-binding domain, a Box B DNA-binding domain, a cryptic nuclear localization signal (NLS), and an acidic tail. The wildtype HMGB1 protein further comprises a receptor for advanced glycation end-products (RAGE) binding domain.

In the context of the wildtype protein, the acidic tail interacts with the Box A and Box B domains, causing the HMGB1 protein to adopt an auto-inhibited conformation that is not competent for DNA binding. The acidic tail therefore suppresses the chromatin remodeling activity of the wildtype HMGB1 protein. In contrast, a truncated HMGB1 protein lacking the acidic tail may remain in the active conformation and thereby exhibit increased chromatin remodeling activity.

One of ordinary skill in the art would appreciate that chromatin structure affects the efficacy of various genome editing techniques, and that target sequences located in chromatin-dense regions, in particular, may be less susceptible to genome editing. For example, the CRISPR-associated nuclease Cas9 exhibits reduced editing of targets whose protospacer adjacent motifs (PAMs) are located within nucleosomes. Thus, truncated HMGB1 proteins may be fused with a programmable DNA-binding protein (e.g., a CRISPR-associated nuclease), thereby conferring increased editing efficiency at loci that are positioned within compacted chromatin. See, e.g., Ding X, Seebeck T, Feng Y, Jiang Y, Davis G D, Chen F. Improving CRISPR-Cas9 Genome Editing Efficiency by Fusion with Chromatin-Modulating Peptides. CRISPR J. 2019 February; 2:51-63.

While truncated HMGB1 proteins lacking an acidic tail may have enhanced chromatin remodeling activity relative to the wildtype HMGB1 protein, other elements within the HMGB1 protein may also promote chromatin remodeling activity. For example, truncated HMGB1 proteins comprising a cryptic NLS, which may or may not have constitutive NLS activity, may exhibit increased chromatin remodeling activity compared to truncated HMGB1 proteins that lack the cryptic NLS.

The present disclosure provides for an HMGB1 polypeptide comprising certain domains of HMGB1. In some embodiments, the system, the fusion protein or a nucleic acid encoding same, comprises a HMGB1 polypeptide, or a nucleic acid encoding same.

In some embodiments, the HMGB1 polypeptide comprises an HMGB1 Box B domain. In some embodiments, the HMGB1 polypeptide comprises at least 7 contiguous HMGB1 amino acid residues C-terminal to the HMGB1 Box B domain. In some embodiments, the HMGB1 polypeptide comprises 7, 8, 9, 10, 11, 12, 13, 14, 15, 16, 17, 18, 19, or 20 contiguous HMGB1 amino acid residues C-terminal to the HMGB1 Box B domain. In some embodiments, the HMGB1 polypeptide comprises at least 7, 8, 9, 10, 11, 12, 13, 14, 15, 16, 17, 18, or 19 contiguous HMGB1 amino acid residues C-terminal to the HMGB1 Box B domain. In some embodiments, the HMGB1 polypeptide comprises 7-10, 7-12, 7-14, 7-16, 7-18, 7-20, 8-10, 8-12, 8-14, 8-16, 8-18, 8-18, 8-20, 10-12, 10-14, 10-16, 10-18, 10-20, 12-14, 12-16, 12-18, 12-20, 14-16, 14-18, 14-20, 16-18, 16-20, or 18-20 contiguous HMGB1 amino acid residues C-terminal to the HMGB1 Box B domain. In some embodiments, the HMGB1 polypeptide comprises 7-9, 7-11, 7-13, 7-15, 7-17, 7-19, 7-20, 9-11, 9-13, 9-15, 9-17, 9-19, 9-20, 11-13, 11-15, 11-17, 11-19, 11-20, 13-15, 13-17, 13-19, 13-20, 15-17, 15-19, 15-20, 17-19, 17-20, or 19-20 contiguous HMGB1 amino acid residues C-terminal to the HMGB1 Box B domain.

In some embodiments, the HMGB1 polypeptide lacks all or part of an HMGB1 acidic tail domain. In some embodiments, the HMGB1 polypeptide lacks all of an acidic tail domain. In some embodiments, the HMGB1 polypeptide lacks part of an acidic tail domain. In some embodiments, the HMGB1 polypeptide comprises an HMGB1 Box B domain and at least 7 contiguous amino acid residues C-terminal to the HMGB1 Box B domain, wherein the HMGB1 polypeptide lacks all or part of an acidic tail domain. In some embodiments, the HMGB1 polypeptide comprises 7, 8, 9, 10, 11, 12, 13, 14, 15, 16, 17, 18, 19, or 20 contiguous amino acid residues C-terminal to the HMGB1 Box B domain. In some embodiments, the HMGB1 polypeptide comprises at least 7, 8, 9, 10, 11, 12, 13, 14, 15, 16, 17, 18, or 19 contiguous amino acid residues C-terminal to the HMGB1 Box B domain. In some embodiments, the HMGB1 polypeptide comprises 7-10, 7-12, 7-14, 7-16, 7-18, 7-20, 8-10, 8-12, 8-14, 8-16, 8-18, 8-18, 8-20, 10-12, 10-14, 10-16, 10-18, 10-20, 12-14, 12-16, 12-18, 12-20, 14-16, 14-18, 14-20, 16-18, 16-20, or 18-20 contiguous amino acid residues C-terminal to the HMGB1 Box B domain. In some embodiments, the HMGB1 polypeptide comprises 7-9, 7-11, 7-13, 7-15, 7-17, 7-19, 7-20, 9-11, 9-13, 9-15, 9-17, 9-19, 9-20, 11-13, 11-15, 11-17, 11-19, 11-20, 13-15, 13-17, 13-19, 13-20, 15-17, 15-19, 15-20, 17-19, 17-20, or 19-20 contiguous amino acid residues C-terminal to the HMGB1 Box B domain.

In some embodiments, the HMGB1 polypeptide comprises a cryptic nuclear localization signal (NLS). In some embodiments, the cryptic NLS comprises the sequence of EKSKKKK (SEQ ID NO: 1787) or a variant thereof. In some embodiments, the HMGB1 polypeptide comprises a receptor for advanced glycation end-products (RAGE) binding domain. In some embodiments, the RAGE binding domain comprises the sequence of SEQ ID NO: 1803. In some embodiments, the HMGB1 polypeptide comprises 7-20 amino acid residues C-terminal to the HMGB1 Box B domain. In some embodiments, the HMGB1 polypeptide comprises 7-20 amino acid residues immediately C-terminal to the HMGB1 Box B domain. In some embodiments, the polypeptide comprises the sequence of SEQ ID NO: 1804.

In some embodiments, the HMGB1 polypeptide lacks an acidic tail domain. In some embodiments, the HMGB1 polypeptide lacks a sequence at least 50%, 60%, 70%, 80%, 90%, 94%, or 97% identical to SEQ ID NO: 1788. In some embodiments, the HMGB1 polypeptide lacks the sequence of SEQ ID NO: 1788. In some embodiments, the HMGB1 polypeptide lacks transcriptional stimulatory function.

In some embodiments, the HMGB1 polypeptide comprises a cryptic NLS that is located C-terminal to the HMGB1 Box B domain. In some embodiments, the HMGB1 polypeptide comprises a cryptic NLS that is located C-terminal to the HMGB1 Box B domain, optionally wherein the cryptic NLS is comprised within the at least 7 contiguous amino acid residues. In some embodiments, the cryptic NLS immediately follows the C-terminal end of the HMGB1 Box B domain. In some embodiments, the HMGB1 polypeptide comprises a cryptic NLS that is located N-terminal to the HMGB1 Box B domain. In some embodiments, the cryptic NLS immediately precedes the N-terminal end of the HMGB1 Box B domain.

In some embodiments, the HMGB1 polypeptide lacks an HMGB1 Box A domain. In some embodiments, the HMGB1 polypeptide comprises two HMGB1 Box B domains. In some embodiments, the cryptic NLS is located C-terminal to the C-terminal end of the two HMGB1 Box B domains. In some embodiments, the HMGB1 polypeptide comprises an HMGB1 Box A domain. In some embodiments, the HMGB1 Box B domain is located C-terminal to the HMGB1 Box A domain.

In some embodiments, the HMGB1 polypeptide comprises, from N-terminus to C-terminus: (1) HMGB1 Box A domain-HMGB1 Box B domain-cryptic NLS; (2) HMGB1 Box B domain-HMGB1 Box B domain-cryptic NLS; or (3) HMGB1 Box B domain-cryptic NLS. In some embodiments, the HMGB1 polypeptide comprises a sequence having at least 80%, 90%, 95%, 98%, or 100% identity to any one of SEQ ID NOs: 1783-1792 and 1803-1804, or is encoded by a nucleic acid having at least 80%, 85%, 90%, 95%, 98% or 100% identity to the sequence of any one of SEQ ID NOs: 1793-1802. In some embodiments, the HMGB1 polypeptide comprises a sequence having at least 80%, 90%, 95%, 98%, or 100% identity to any one of SEQ ID NOs: 1783-1792 and 1803-1804. In some embodiments, the HMGB1 polypeptide comprises a sequence that is encoded by a nucleic acid having at least 80%, 85%, 90%, 95%, 98% or 100% identity to the sequence of any one of SEQ ID NOs: 1793-1802.

In some embodiments, the HMGB1 Box A domain comprises amino acid residues 2-78 of SEQ ID NO: 1783. In some embodiments, the HMGB1 Box A domain comprises amino acid residues 2-84 of SEQ ID NO: 1783. In some embodiments, the HMGB1 Box A domain comprises amino acid residues 2-88 of SEQ ID NO: 1783. In some embodiments, the HMGB1 Box B domain comprises amino acid residues 89-162 of SEQ ID NO: 1783. In some embodiments, the HMGB1 Box B domain comprises amino acid residues 85-185 of SEQ ID NO: 1783, including the cryptic nuclear localization signal. In some embodiments, the HMGB1 Box B domain consists of amino acid residues 85-185, including the cryptic nuclear localization signal.

In some embodiments, the HMGB1 polypeptide comprises an HMGB1 Box B domain and at least 7 amino acid residues C-terminal to the HMGB1 Box B domain, and wherein the HMGB1 polypeptide lacks all or part of an acidic tail domain. In some embodiments, the acidic tail domain comprises amino acid residues 186-215 relative to SEQ ID NO: 1783. In some embodiments, the cryptic NLS comprises the sequence of EKSKKKK (SEQ ID NO: 1787) or a variant thereof. In some embodiments, the HMGB1 polypeptide comprises an HMGB1 Box B domain and lacking an acidic tail domain, further wherein the HMGB1 polypeptide comprises a cryptic NLS comprising amino acid residues 179-185 of SEQ ID NO: 1783 or a variant thereof. In some embodiments, the HMGB1 polypeptide comprises an HMGB1 Box B domain and lacking an acidic tail domain, further wherein the HMGB1 polypeptide comprises amino acid residues 166-185 of SEQ ID NO: 1783, which comprises the cryptic NLS (SEQ ID NO: 1787), or a variant of amino acid residues 166-185 of SEQ ID NO: 1783.

In some embodiments, the cryptic NLS is located C-terminal to the HMGB1 Box B domain. In some embodiments, the cryptic NLS immediately follows the C-terminal end of the HMGB1 Box B domain. In some embodiments, the cryptic NLS is located N-terminal to the HMGB1 Box B domain. In some embodiments, the cryptic NLS immediately precedes the C-terminal end of the HMGB1 Box B domain.

In some embodiments, the sequence of EKSKKKK (SEQ ID NO: 1787) is located C-terminal to the HMGB1 Box B domain. In some embodiments, the sequence of EKSKKKK (SEQ ID NO: 1787) immediately follows the C-terminal end of the HMGB1 Box B domain. In some embodiments, the sequence of EKSKKKK (SEQ ID NO: 1787) is located N-terminal to the HMGB1 Box B domain. In some embodiments, the sequence of EKSKKKK (SEQ ID NO: 1787) immediately precedes the C-terminal end of the HMGB1 Box B domain.

In some embodiments, the HMGB1 polypeptide comprises the sequence of amino acid residues 166-185 relative to SEQ ID NO: 1783, which comprises the cryptic NLS (SEQ ID NO: 1787). In some embodiments, the HMGB1 polypeptide of amino acid residues 166-185 relative to SEQ ID NO: 1783 has 2, 1, or 0 amino acid substitutions. In some embodiments, the amino acid substitutions are conservative amino acid substitutions.

In some embodiments, the HMGB1 polypeptide lacks an HMGB1 Box A domain. In some embodiments, the HMGB1 polypeptide comprises two HMGB1 Box B domains. In some embodiments, wherein the HMGB1 polypeptide comprises two HMGB1 Box B domains, the cryptic NLS is located C-terminal to the most C-terminal HMGB1 Box B domain. In some embodiments, wherein the HMGB1 polypeptide comprises two HMGB1 Box B domains, the sequence of EKSKKKK (SEQ ID NO: 1787) is located C-terminal to the most C-terminal HMGB1 Box B domain. In some embodiments, the HMGB1 polypeptide comprises an HMGB1 Box A domain. In some embodiments, wherein the HMGB1 polypeptide comprises an HMGB1 Box A domain, the HMGB1 Box B domain is located C-terminal to the HMGB1 Box A domain.

In some embodiments, amino acid residues 166-185 relative to SEQ ID NO: 1783 is present, the domain are located C-terminal to the most C-terminal Box B domain. When only one HMGB1 Box B domain is present, it is understood to be the most C-terminal HMGB1 Box B domain.

In some embodiments, the HMGB1 polypeptide comprises a sequence having at least 80%, 90%, 95%, 98%, or 100% identity to any one of SEQ ID NOs: 1783-1792 and 1803-1804, or that is encoded by a nucleic acid having at least 80%, 85%, 90%, 95%, 98% or 100% identity to the sequence of any one of SEQ ID NOs: 1793-1802.

In some embodiments, the HMGB1 polypeptide comprises a sequence having at least 80%, 90%, 95%, 98%, or 100% identity to SEQ ID NO: 1789 or that is encoded by a nucleic acid having at least 80%, 85%, 90%, 95%, 98% or 100% identity to SEQ ID NO: 1799.

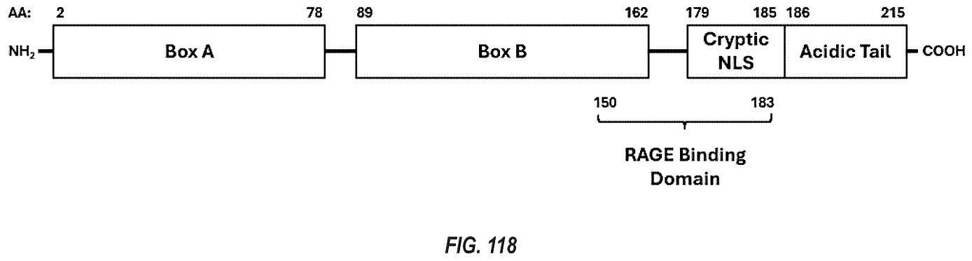

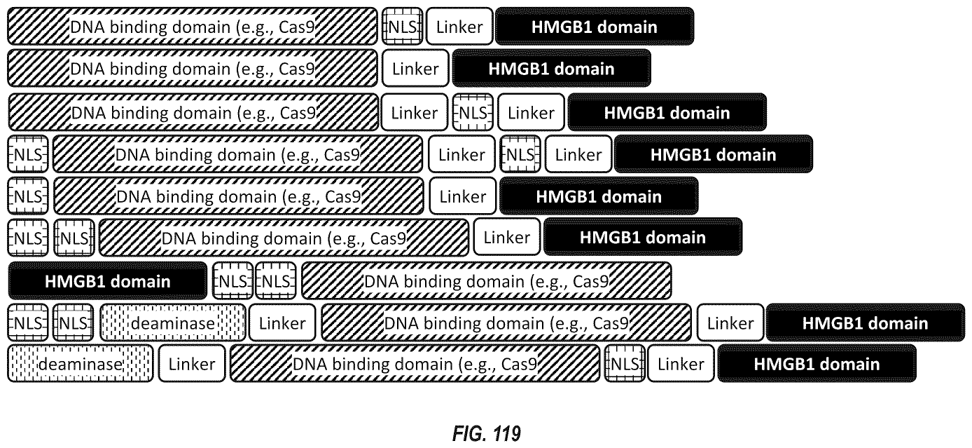

In some embodiments, the system or fusion protein disclosed herein comprises a programmable DNA-binding protein and an HMGB1 polypeptide comprising an HMGB1 Box B domain and lacking an acid tail domain. In some embodiments, the HMGB1 polypeptide comprises a cryptic NLS. In some embodiments, the cryptic NLS comprises the sequence of EKSKKK (SEQ ID NO: 1787). In some embodiments, the programmable DNA-binding protein is located N-terminal to the HMGB1 polypeptide. In some embodiments, the programmable DNA-binding protein is located C-terminal to the HMGB1 domain. In some embodiments, the system or fusion protein comprises at least one heterologous nuclear localization signal (NLS). See, e.g., FIG. 118, which shows certain non-limiting, exemplary HMGB1-programmable DNA-binding protein fusion proteins.

In some embodiments, the nCas9 and the DNA-dependent DNA polymerase are covalently linked sequentially in a polypeptide comprising from N-terminus to C-terminus a HMGB1 polypeptide, the nCas9, and the DNA-dependent DNA polymerase. In some embodiments, the nCas9 and the DNA-dependent DNA polymerase are covalently linked sequentially in a polypeptide comprising from N-terminus to C-terminus the nCas9, a HMGB1 polypeptide, and the DNA-dependent DNA polymerase. In some embodiments, the nCas9 and the DNA-dependent DNA polymerase are covalently linked sequentially in a polypeptide comprising from N-terminus to C-terminus a nCas9, the DNA-dependent DNA polymerase, and a HMGB1 polypeptide.

In some embodiments, the nCas9 and the DNA-dependent DNA polymerase are covalently linked sequentially in a polypeptide from N-terminus to C-terminus, wherein the order is nuclear localization signal-HMGB1 polypeptide-nCas9-DNA Dependent DNA polymerase-nuclear localization signal. In some embodiments, the nCas9 and the DNA-dependent DNA polymerase are covalently linked sequentially in a polypeptide from N-terminus to C-terminus, wherein the order is nuclear localization signal-nCas9-HMGB1 polypeptide-DNA Dependent DNA polymerase-nuclear localization signal. In some embodiments, the nCas9 and the DNA-dependent DNA polymerase and are covalently linked sequentially in a polypeptide from N-terminus to C-terminus, wherein the order is nuclear localization signal-nCas9-DNA Dependent DNA polymerase-HMGB1 polypeptide-nuclear localization signal. In some embodiments, the polypeptide fusion further comprises one or more linkers between the nuclear localization signal(s), nCas9, DNA-dependent DNA polymerase, and/or the HMGB1 polypeptide.

### UTRs; Kozak Sequences

In some embodiments, the nucleic acid (e.g., mRNA) disclosed herein comprises a 5′ UTR, 3′ UTR, or 5′ and 3′ UTRs from Hydroxysteroid 17-Beta Dehydrogenase 4 (HSD17B4 or HSD) or globin such as human alpha globin (HBA), human beta globin (HBB), Xenopus laevis beta globin (XBG), bovine growth hormone, cytomegalovirus (CMV), mouse Hba-al, heat shock protein 90 (Hsp90), glyceraldehyde 3-phosphate dehydrogenase (GAPDH), beta-actin, alpha-tubulin, tumor protein (p53), or epidermal growth factor receptor (EGFR).

In some embodiments, the mRNA comprises a 5′ UTR selected from 5′ XBG, Apt17, 4EApt1, PABPv3, NELL2, Markup2, and ANKRD1. In some embodiments, the 5′ UTR comprises 5′ XBG. In some embodiments, the 5′ UTR comprises 4Eapt1. In some embodiments, the 5′ UTR comprises 4Gapt1. In some embodiments, the 5′ UTR comprises PABPv3. In some embodiments, the 5′ UTR comprises Apt17 and 5′ XBG. In some embodiments, the 5′ UTR comprises Apt17, PABPv3, and 5′ XBG. In some embodiments, the 5′ UTR comprises 4Gapt1 and 5′XBG. In some embodiments, the 5′ UTR comprises 4Gapt1, PABPv3, and 5′XBG and NELL2. In some embodiments, the 5′ UTR comprises 4Gapt1 and Markup2. In some embodiments, the 5′ UTR comprises 4Eapt1 and 5′XBG. Non-limiting exemplary sequences of the 5′ UTR are provided in Table 4A and Table 6. In some embodiments, the 5′ UTR comprises a nucleic acid sequence selected from any one of SEQ ID NOs 1720-1723.

In some embodiments, mRNA comprises a 3′ UTR selected from 3′ XBG or xr-RNA. In some embodiments, the 3′ UTR is 3′ XBG. In some embodiments, the 3′ UTR is xr-RNA. Non-limiting exemplary sequences of the 5′ UTR are provided in Table 4A and Table 6. In some embodiments, the 5′ UTR comprises a nucleic acid sequence selected from any one of SEQ ID NOs: 1684 and 1740.

In some embodiments, the nucleic acid described herein does not comprise a 5′ UTR, e.g., there are no additional nucleotides between the 5′ cap and the start codon. In some embodiments, the nucleic acid comprises a Kozak sequence (described below) between the 5′ cap and the start codon, but does not have any additional 5′ UTR. In some embodiments, the nucleic acid does not comprise a 3′ UTR, e.g., there are no additional nucleotides between the stop codon and the poly-A tail.

In some embodiments, the polynucleotide comprises a 5′ UTR with at least 85%, 90%, or 95% identity to any one of SEQ ID NOs: 1674-1681. In some embodiments, the polynucleotide comprises a 3′ UTR with at least 85%, 90%, or 95% identity to any one of SEQ ID NOs: 1682-1689. In some embodiments, the polynucleotide comprises a 5′ UTR and 3′ UTR from the same source.

In some embodiments, the nucleic acid herein comprises a Kozak sequence. The Kozak sequence can affect translation initiation and the overall yield of a polypeptide translated from an mRNA. A Kozak sequence includes a methionine codon that can function as the start codon. A minimal Kozak sequence is NNNRUGN wherein at least one of the following is true: the first N is A or G and the second N is G. In the context of a nucleotide sequence, R means a purine (A or G). In some embodiments, the Kozak sequence is RNNRUGN, NNNRUGG, RNNRUGG, RNNAUGN, NNNAUGG, RNNAUGG, or GCCACCAUG. In some embodiments, the Kozak sequence is Kozak VI (SEQ ID NO: 1724). In some embodiments, the Kozak sequence is Kozak V2 (SEQ ID NO: 1725).

### Poly-A Tails

In some embodiments, the nucleic acid disclosed herein further comprises a poly-adenylated (poly-A) tail sequence or a polyadenylation signal sequence. The poly-A tails may comprise at least 8 consecutive adenine nucleotides, but also comprise one or more non-adenine nucleotide. As used herein, “non-adenine nucleotides” refers to any natural or non-natural nucleotides that do not comprise adenine. Guanine, thymine, and cytosine nucleotides are exemplary non-adenine nucleotides. Thus, the poly-A tails on the nucleic acid described herein may comprise consecutive adenine nucleotides located 3′ to nucleotides encoding a polypeptide of interest. In some instances, the poly-A tails on the nucleic acid comprise non-consecutive adenine nucleotides located 3′ to nucleotides encoding the polypeptide, wherein non-adenine nucleotides interrupt the adenine nucleotides at regular or irregularly spaced intervals.

In some embodiments, the poly-A tail sequence is an encoded poly-A tail sequence. In some embodiments, the poly-A tail sequence is not encoded, optionally wherein the poly-A tail sequence is added enzymatically. In some embodiments, the poly-A tail sequence is added enzymatically.

In some embodiments, the poly-A tail is encoded in a plasmid used for in vitro transcription of an mRNA and becomes part of the transcript. The poly-A sequence encoded in the plasmid, i.e., the number of consecutive adenine nucleotides in the poly-A sequence, may not be exact, e.g., a 100 poly-A sequence in the plasmid may not result in a precisely 100 poly-A sequence in the transcribed mRNA. In some embodiments, the poly-A tail is not encoded in the plasmid, and is added by PCR tailing or enzymatic tailing, e.g., using E. coli poly(A) polymerase.

In some embodiments, the poly-A tail sequence comprises non-adenine nucleotides. In some embodiments, one or more non-adenine nucleotides are positioned to interrupt the consecutive adenine nucleotides so that a poly(A) binding protein can bind to a stretch of consecutive adenine nucleotides. In some embodiments, one or more non-adenine nucleotide(s) is located after at least 8, 9, 10, 11, or 12 consecutive adenine nucleotides. In some embodiments, the one or more non-adenine nucleotide is located after 8-50 consecutive adenine nucleotides. In some embodiments, the one or more non-adenine nucleotide is located after 8-100 consecutive adenine nucleotides.

In some embodiments, the poly-A tail comprises or contains one non-adenine nucleotide or one consecutive stretch of 2-10 non-adenine nucleotides.

In some embodiments, the non-adenine nucleotide is guanine, cytosine, or thymine. In some instances, where more than one non-adenine nucleotide is present, the non-adenine nucleotide may be selected from: a) guanine and thymine nucleotides; b) guanine and cytosine nucleotides; c) thymine and cytosine nucleotides; or d) guanine, thymine and cytosine nucleotides.

In some embodiments, the poly-A tail sequence comprises 100-400 nucleotides.

In some embodiments, the poly-A tail sequence comprises a sequence of SEQ ID NO: 1692 or 1693.

In some embodiments, the poly-A tail sequence comprises a sequence of SEQ ID NO: 1741 or 1742.

In one embodiment, the poly-A tail sequence comprises SEQ ID NO: 1741. In one embodiment, the poly-A tail sequence comprises SEQ ID NO: 1742.

### Modified Nucleotides

In some embodiments, the ORF or mRNA disclosed herein comprises a modified uridine at some or all uridine positions. In some embodiments, the modified uridine is a uridine modified at the 5 position, e.g., with a halogen or C1-C3 alkoxy. In some embodiments, the modified uridine is a pseudouridine modified at the 1 position, e.g., with a C1-C3 alkyl. The modified uridine can be, for example, pseudouridine, N1-methyl-pseudouridine, 5-methoxyuridine, 5-iodouridine, or a combination thereof.

In some embodiments, at least 10%, 20%, 30%, 40%, 50%, 60%, 70%, 80%, 90%, or 100% of the uridine positions in the nucleic acid disclosed herein are modified uridines. In some embodiments, the uridine positions in an mRNA disclosed herein are modified uridines, e.g., 5-methoxyuridine, 5-iodouridine, N1-methyl pseudouridine, pseudouridine, or a combination thereof. In some embodiments, 80-95% or 80-100% of the uridine positions in an mRNA disclosed herein are modified uridines, e.g., 5-methoxyuridine, 5-iodouridine, N1-methyl pseudouridine, or pseudouridine.

In some embodiments, the modified uridine is one or more of N1-methyl-pseudouridine, pseudouridine, 5-methoxyuridine, or 5-iodouridine. In some embodiments, the modified uridine is N1-methyl-pseudouridine or 5-methoxyuridine. In some embodiments, the modified uridine is N1-methyl-pseudouridine. In some embodiments, the modified uridine is pseudouridine. In some embodiments, the modified uridine is 5-methoxyuridine. In some embodiments, the modified uridine is 5-iodouridine. In some embodiments, at least 80% of the uridine is substituted with the modified uridine. In some embodiments, 100% uridine is substituted with the modified uridine.

### 5′ Caps

In some embodiments, the nucleotide sequence encoding the SpyCas9 nickase, DNA-dependent DNA polymerase, or SpyCas9 nickase and DNA-dependent DNA polymerase fusion protein comprises a 5′ cap selected from Cap0, Cap1, and Cap2. In some embodiments, the 5′ cap is Cap0. In some embodiments, the 5′ cap is Cap1.

A 5′ cap is generally a 7-methylguanine ribonucleotide (which may be further modified, as discussed below e.g., with respect to ARCA) linked through a 5′-triphosphate to the 5′ position of the first nucleotide of the 5′-to-3′ chain of the nucleic acid, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the nucleic acid comprise a 2′-methoxy and a 2′-hydroxyl, respectively. In Cap2, the riboses of the first and second cap-proximal nucleotides of the nucleic acid both comprise a 2′-methoxy. See, e.g., Katibah et al. (2014) Proc Natl Acad Sci USA 111(33):12025-30; Abbas et al. (2017) Proc Natl Acad Sci USA 114(11):E2106-E2115. Most endogenous higher eukaryotic nucleic acids, including mammalian nucleic acids such as human nucleic acids, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of a nucleic acids with a cap other than Cap1 or Cap2, potentially inhibiting translation of the nucleic acid.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap or a Cap0-like cap in which the 2′ position of the first cap-proximal nucleotide is hydroxyl. See, e.g., Stepinski et al., (2001) “Synthesis and properties of mRNAs containing the novel ‘anti-reverse’ cap analogs 7′methyl (3′-O-methyl)GpppG and 7′methyl(3′deoxy)GpppG,” RNA 7:1486-1495. The ARCA structure is shown below.

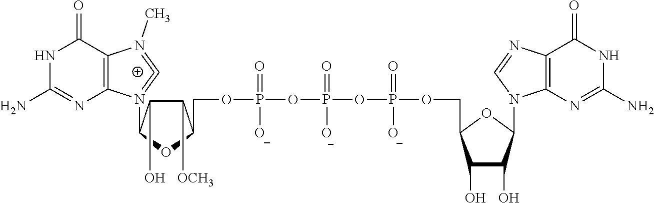

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively. The CleanCap™ AG structure is shown below. CleanCap™ structures are sometimes referred to herein using the last three digits of the catalog numbers listed above (e.g., “CleanCap™ 113” for TriLink Biotechnologies Cat. No. N-7113).

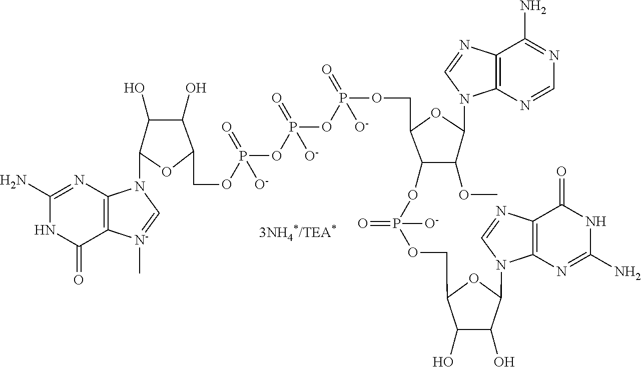

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479. For additional discussion of caps and capping approaches, see, e.g., WO2017/053297 and Ishikawa et al., Nucl. Acids. Symp. Ser. (2009) No. 53, 129-130.

### Exemplary Compositions and Systems

Provided herein are systems comprising (a) a SpyCas9 nuclease nickase or a nucleic acid encoding the same, (b) a DNA-dependent DNA polymerase or a nucleic acid encoding the same, and (c) a template guide RNA for template-based genome editing. In some embodiments, the system further comprises an enhancer of template-based genome editing, also referred to herein as an enhancer, e.g., a Vpx, BGLF4, M97, KSHV ORF36, or FEN1 protein. In some embodiments the enhancers of template-based genome editing are Vpx and FEN1. In some embodiments, the system further comprises a heterodimerization domain, optionally a self-assembling coiled coil heterodimerization domain, e.g., a KI domain and an EI domain, in each the DNA-dependent DNA polymerase and in the enhancer to promote heterodimerization of the DNA-dependent DNA polymerase and the enhancer. Additional exemplary heterodimerization domains include, but are not limited to: leucine zipper domains; PDZ domains; streptavidin and a streptavidin-binding domain; foldon domains; hydrophobic polypeptides; or an antibody or one or more binding fragments thereof (e.g., scFv, VHH domain) on one component that binds an epitope, either naturally occurring or inserted, in the other component. In some embodiments, the position of the heterodimer domain in the first and second polypeptides is independently selected.

In some embodiments, the enhancer comprising a heterodimerization domain is a FEN1 protein.

In some embodiments, the system comprises (a) a single polypeptide comprising a SpyCas9 nickase and a DNA-dependent DNA polymerase; and (b) a single polynucleotide comprising, from 5′ to 3′, (i) a SpyCas9 guide RNA comprising a spacer and a scaffold; and (ii) a template sequence and a DRS. In some embodiments, the composition comprises (a) a single polypeptide comprising a SpyCas9 nickase and a DNA-dependent DNA polymerase; (b) a first polynucleotide comprising a SpyCas9 guide RNA comprising, from 5′ to 3′, a spacer and a scaffold; and (c) a second polynucleotide comprising, from 5′ to 3′, a template sequence and a DRS, wherein the first and second polynucleotide are provided as separate molecules, optionally wherein the second polynucleotide comprises an affinity tag, optionally an aptamer, for binding to the DNA-dependent DNA polymerase. In some embodiments, the system further comprises an enhancer of template-based genome editing, also referred to herein as an enhancer. In some embodiments, the system further comprises a heterodimerization domain, optionally a self-assembling coiled coil heterodimerization domain, e.g., a KI domain and an EI domain, in each the DNA-dependent DNA polymerase and in the enhancer to promote heterodimerization of the DNA-dependent DNA polymerase and the enhancer.

In some embodiments, the DNA-dependent DNA polymerase and nCas9 are covalently linked sequentially in a polypeptide from N-terminus to C-terminus, wherein the order is NLS-nCas9-linker-NLS-linker-DNA-dependent DNA polymerase-NLS-NLS-linker-heterodimerization domain. In some embodiments, the DNA-dependent DNA polymerase and nCas9 are covalently linked sequentially in a polypeptide from N-terminus to C-terminus, wherein the order is NLS-nCas9-linker-NLS-linker-DNA-dependent DNA polymerase-linker-NLS-linker-heterodimerization domain. In further embodiments, the heterodimerization domain comprises a coiled coil heterodimerization domain. In further embodiments, the heterodimerization domain comprises an EV, EI, KV, or KI domain. In further embodiments, the heterodimerization domain comprises an EI or KI domain. In further embodiments, the heterodimerization domain comprises SEQ ID NO: 1589, SEQ ID NO: 1593, or SEQ ID NO: 1595.

In some embodiments, the DNA-dependent DNA polymerase and nCas9 are covalently linked sequentially in a polypeptide from N-terminus to C-terminus, wherein the order is: (a) NLS-nCas9-linker-NLS-linker-DNA-dependent DNA polymerase-NLS-NLS-linker-EI domain; or (b) NLS-nCas9-linker-NLS-linker-DNA-dependent DNA polymerase-NLS-NLS-linker-KI domain.

In some embodiments, the DNA-dependent DNA polymerase and nCas9 are covalently linked sequentially in a polypeptide from N-terminus to C-terminus in an order of: (a) NLS-nCas9-linker-NLS-linker-polK (1-526, GE411-412RV)-linker-NLS-linker-EI domain; (b) NLS-nCas9-linker-NLS-linker-polK (1-526, GE411-412RV)-linker-NLS-linker-KI domain; (c) NLS-nCas9-linker-NLS-linker-T5Pol-linker-NLS-NLS-linker-EI domain; or (d) NLS-nCas9-linker-NLS-linker-T5Pol-linker-NLS-NLS-linker-KI domain.

In some embodiments, the DNA-dependent DNA polymerase comprises human DNA polymerase K (polK), cynomolgus polK, mouse polK, phage T5 DNA polymerase (T5 pol), DNA polymerase Θ, E. coli DNA polymerase I, optionally wherein the E. coli dependent DNA polymerase is a Klenow(exo-), or DNA polymerase N.

In some embodiments, the system comprises an nCas9-polymerase fusion protein, wherein the polymerase is selected from: polymerase Θ, E. coli polymerase I, polymerase N, or Phi29 polymerase, wherein (a) the polymerase Θ and nCas9 are covalently linked sequentially in a polypeptide from N-terminus to C-terminus in an order selected from: (i) nCas9-polymerase Θ-NLS, or (ii) nCas9-linker-polymerase Θ-linker-NLS; or (b) the E. coli polymerase I comprising Klenow(exo-) and nCas9 are covalently linked sequentially in a polypeptide from N-terminus to C-terminus in an order selected from: (i) nCas9-Klenow(exo-)-NLS; (ii) nCas9-linker-Klenow(exo-)-linker-NLS; (iii) NLS-nCas9-NLS-E coli PolI; or (iv) NLS-nCas9-linker-NLS-linker-E coli PolI; or (c) the polymerase N and nCas9 are covalently linked sequentially in a polypeptide from N-terminus to C-terminus in an order selected from: (i) polymerase N-NLS-nCas9-NLS; or (ii) polymerase N-linker-NLS-nCas9-linker-NLS; (d) the Phi29 polymerase and nCas9 are covalently linked sequentially in a polypeptide from N-terminus to C-terminus in an order selected from: (i) NLS-nCas9-NLS-Phi29 polymerase; (ii) NLS-nCas9-linker-NLS-linker-Phi29 polymerase; (iii) nCas9-Phi29 polymerase-NLS; or (iv) nCas9-linker-Phi29 polymerase-linker-NLS. In some embodiments, the fusion protein comprises a linker or further comprises an additional linker. In some embodiments, the fusion protein comprises an amino acid sequence that is at least 90%, 91%, 92%, 93%, 94%, 95%, 96%, 97%, 98%, 99%, or 100% identical to any one of SEQ ID NOs: 1024, 1027, 1030, 1033, 1036, 1039, 1042, 1045, 1048, 1053, 1056, 1059, 1074, 1081, 1084, 1086, 1094, 1097, 1100, 1102, 1112, 1114, 1124, 1129, 1134, 1137, 1142, 1147, 1152, 1157, 1162, 1167, 1172, 1192, 1195, 1198, 1219, 1224, 1229, 1234, 1236, 1239, 1254, and 1259, 1268, and 1273, and 1276, 1300, 1303, 1307, 1373, 1376, 1388, 1447, 1450, 1453, 1923, 1926, 1929, 1932, 1935, and 1938, or a nucleic acid encoding the fusion protein sequence of any one of SEQ ID NOs: 1024, 1027, 1030, 1033, 1036, 1039, 1042, 1045, 1048, 1059, 1053, 1056, 1059, 1074, 1081, 1084, 1086, 1094, 1097, 1100, 1102, 1112, 1114, 1124, 1129, 1134, 1137, 1142, 1147, 1152, 1157, 1162, 1167, 1172, 1192, 1195, 1198, 1219, 1224, 1229, 1234, 1236, 1239, 1254, and 1259, 1268, and 1273, 1276, 1300, 1303, 1307, 1373, 1376, 1388, 1477, 1450, 1453, 1923, 1926, 1929, 1932, 1935, and 1938.

In some embodiments, the DNA-dependent DNA polymerase is a human DNA polymerase. In some embodiments, the DNA-dependent DNA polymerase is not a Phi29 DNA polymerase. In some embodiments, the DNA-dependent DNA polymerase does not comprise at least 5 point mutations. In some embodiments, the DNA-dependent polymerase does not comprise at least 8 point mutations. In some embodiments, the wild-type DNA-dependent DNA polymerase has a polymerization rate at least twice as high, optionally at least five times as high at 37° C. as compared to 30° C.

In some embodiments, the system comprises a human DNA polymerase kappa (polK). In some embodiments, the human polk comprises an amino acid sequence that has at least 90%, 91%, 92%, 93%, 94%, 95%, 96%, 97%, 98%, 99%, or 100% identity to amino acid residues 19-526 of SEQ ID NO: 1021; or an amino acid sequence that has at least 90%, 91%, 92%, 93%, 94%, 95%, 96%, 97%, 98%, 99%, or 100% identity to SEQ ID NO: 1021 or 1206. In some embodiments, the polK comprises amino acids 19-526, optionally amino acids 1-526, of polK relative to SEQ ID NO: 1021. In some embodiments, the polk comprises a mutation at G411 or E412 relative to SEQ ID NO: 1021, optionally wherein the polk comprises a mutation at G411 and E412. In some embodiments, the polk comprises GE411-412RV mutations relative to SEQ ID NO: 1021. In some embodiments, polk is encoded by: (a) a nucleotide sequence that has at least 90%, 91%, 92%, 93%, 94%, 95%, 96%, 97%, 98%, 99%, or 100% identity to a coding sequence for amino acid residues 19-526 within SEQ ID NO: 1020; (b) a nucleotide sequence that has at least 90%, 91%, 92%, 93%, 94%, 95%, 96%, 97%, 98%, 99%, or 100% identity to SEQ ID NO: 1020; (c) a human DNA polymerase K that is encoded by a nucleotide sequence that has at least 90%, 91%, 92%, 93%, 94%, 95%, 96%, 97%, 98%, 99%, or 100% identity to SEQ ID NO: 1201; (d) a nucleotide sequence that has at least 90%, 91%, 92%, 93%, 94%, 95%, 96%, 97%, 98%, 99%, or 100% identity to SEQ ID NO: 1203; (e) a nucleotide sequence that has at least 90%, 91%, 92%, 93%, 94%, 95%, 96%, 97%, 98%, 99%, or 100% identity to SEQ ID NO: 1205; (f) a nucleotide sequence that has at least 90%, 91%, 92%, 93%, 94%, 95%, 96%, 97%, 98%, 99%, or 100% identity to SEQ ID NO: 1264; or (g) a nucleotide sequence that has at least 90%, 91%, 92%, 93%, 94%, 95%, 96%, 97%, 98%, 99%, or 100% identity to SEQ ID NO: 1018. In some embodiments, the polK is encoded by a nucleotide sequence that has at least 90%, 91%, 92%, 93%, 94%, 95%, 96%, 97%, 98%, 99%, or 100% identity to SEQ ID NO: 1020. In some embodiments, the polk is encoded by a nucleotide sequence that has at least 90%, 91%, 92%, 93%, 94%, 95%, 96%, 97%, 98%, 99%, or 100% identity to SEQ ID NO: 1201. In some embodiments, the polK is encoded by a nucleotide sequence that has at least 90%, 91%, 92%, 93%, 94%, 95%, 96%, 97%, 98%, 99%, or 100% identity to SEQ ID NO: 1205.

In some embodiments, the system comprises a cynomolgus DNA polymerase kappa (polK). In some embodiments, the cynomolgus polK comprises an amino acid sequence that has at least 90%, 91%, 92%, 93%, 94%, 95%, 96%, 97%, 98%, 99%, or 100% identity to amino acid residues 1-526 of SEQ ID NO: 1261; or an amino acid sequence that has at least 90%, 91%, 92%, 93%, 94%, 95%, 96%, 97%, 98%, 99%, or 100% identity to SEQ ID NO: 1261. In some embodiments, the polk comprises amino acids 1-526 of polK relative to SEQ ID NO: 1261. In some embodiments, the polK comprises a mutation at G411 or E412 relative to SEQ ID NO: 1261, optionally wherein the polk comprises a mutation at G411 and E412. In some embodiments, the polk comprises GE411-412RV mutations relative to SEQ ID NO: 1261. In some embodiments, polK is encoded by: (a) a nucleotide sequence that has at least 90%, 91%, 92%, 93%, 94%, 95%, 96%, 97%, 98%, 99%, or 100% identity to a coding sequence for amino acid residues 1-526 within SEQ ID NO: 1260; (b) a nucleotide sequence that has at least 90%, 91%, 92%, 93%, 94%, 95%, 96%, 97%, 98%, 99%, or 100% identity to SEQ ID NO: 1260; or (c) a cynomolgus DNA polymerase K that is encoded by a nucleotide sequence that has at least 90%, 91%, 92%, 93%, 94%, 95%, 96%, 97%, 98%, 99%, or 100% identity to SEQ ID NO: 1260.

In some embodiments, the system comprises a mouse DNA polymerase kappa (polK). In some embodiments, the mouse polK comprises an amino acid sequence that has at least 90%, 91%, 92%, 93%, 94%, 95%, 96%, 97%, 98%, 99%, or 100% identity to amino acid residues 1-525 of SEQ ID NO: 1263; or an amino acid sequence that has at least 90%, 91%, 92%, 93%, 94%, 95%, 96%, 97%, 98%, 99%, or 100% identity to SEQ ID NO: 1263. In some embodiments, the polK comprises amino acids 1-525 of polK relative to SEQ ID NO: 1263. In some embodiments, the polK comprises a mutation at G410 or E411 relative to SEQ ID NO: 1263, optionally wherein the polK comprises a mutation at G410 and E411. In some embodiments, the polk comprises GE410-411RV mutations relative to SEQ ID NO: 1263. In some embodiments, polk is encoded by: (a) a nucleotide sequence that has at least 90%, 91%, 92%, 93%, 94%, 95%, 96%, 97%, 98%, 99%, or 100% identity to a coding sequence for amino acid residues 1-525 within SEQ ID NO: 1262; (b) a nucleotide sequence that has at least 90%, 91%, 92%, 93%, 94%, 95%, 96%, 97%, 98%, 99%, or 100% identity to SEQ ID NO: 1262; or (c) a mouse DNA polymerase K that is encoded by a nucleotide sequence that has at least 90%, 91%, 92%, 93%, 94%, 95%, 96%, 97%, 98%, 99%, or 100% identity to SEQ ID NO: 1262.

In some embodiments, the polk and nCas9 are covalently linked sequentially in a polypeptide from N-terminus to C-terminus in an order selected from (a) NLS-nCas9-NLS-polK; (b) NLS-nCas9-linker-NLS-linker-polK; (c) polK-NLS-nCas9-NLS; (d) polK-linker-NLS-linker-nCas9-linker-NLS; (e) NLS-nCas9-NLS-polK (GE411-412RV); (f) NLS-nCas9-linker-NLS-polK (GE411-412RV); (g) NLS-nCas9-NLS-polK (19-526); (h) NLS-nCas9-linker-NLS-linker-polK (19-526); (i) polK (19-526)-NLS-nCas9-NLS; (j) polK (19-526)-Linker-NLS-linker-nCas9-linker-NLS; (k) polK (GE411-412RV)-NLS-NLS-nCas9; (1) polK (GE411-412RV)-linker-NLS-linker-NLS-nCas9; (m) NLS-nCas9-NLS-polK (19-526, GE411-412RV); (n) NLS-nCas9-linker-NLS-linker-polK (19-526, GE411-412RV); (o) NLS-nCas9-NLS-polK-NLS; (p) NLS-nCas9-linker-NLS-linker-polK-linker-NLS; (q) NLS-nCas9-NLS-polK (1-526, GE411-412RV)-NLS; or (r) NLS-nCas9-linker-NLS-linker-polK (1-526, GE411-412RV)-linker-NLS; (s) nCas9-polK (1-526); (t) polK (1-526, GE411-412RV)-nCas9-NLS-heterodimerization domain, optionally wherein the heterodimerization domain comprises a coiled coil heterodimerization domain, optionally an EI domain or KI domain; (u) polK (1-526, GE411-412RV)-linker-NLS-nCas9-linker-NLS-heterodimerization domain, optionally wherein the heterodimerization domain comprises a coiled coil heterodimerization domain, optionally an EI domain or a KI domain; optionally wherein the order is NLS-nCas9-linker-NLS-linker-polK (1-526, GE411-412RV)-linker-NLS, optionally further comprising a heterodimerization domain comprising a coiled coil heterodimerization domain, optionally an EI domain or a KI domain. In some embodiments, wherein the polK and nCa9s are covalently linked sequentially in a polypeptide from N-terminus to C-terminus, the order is: (a) NLS-nCas9-linker-NLS-linker-polK; or (b) NLS-nCas9-linker-NLS-linker-polK-linker-NLS-linker-heterodimerization domain. In some embodiments, wherein the polk and nCa9s are covalently linked sequentially in a polypeptide from N-terminus to C-terminus, the order is: (a) NLS-nCas9-linker-NLS-linker-polK; or (b) NLS-nCas9-linker-NLS-linker-polK-linker-NLS-linker-KI domain. In some embodiments, wherein polK and nCas9 are covalently linked sequentially in a polypeptide from N-terminus to C-terminus, the order is NLS-nCas9-linker-NLS-linker-polK (1-526, GE411-412RV)-linker-NLS-linker-heterodimerization domain. In some embodiments, wherein polK and nCas9 are covalently linked sequentially in a polypeptide from N-terminus to C-terminus, the order is NLS-nCas9-linker-NLS-linker-polK (1-526, GE411-412RV)-linker-NLS-linker-EI domain; or NLS-nCas9-linker-NLS-linker-polK (1-526, GE411-412RV)-linker-NLS-linker-KI domain. In some embodiments, wherein polK and nCas9 are covalently linked sequentially in a polypeptide from N-terminus to C-terminus, the order is: (a) SV40 NLS-nCas9-linker-SV40 NLS-linker-polK (1-526, GE411-412RV)-linker-SV40NLS; (b) SV40 NLS-nCas9-linker-SV40 NLS-linker-polK (1-526, GE411-412RV)-linker-SV40 NLS-linker-EI domain; or (c) SV40 NLS-nCas9-linker-SV40 NLS-linker-polK (1-526, GE411-412RV)-linker-SV40 NLS-linker-KI domain.

In some embodiments, the system comprises (a) a polK polymerase and a SpyCas9 nickase (nCas9), wherein the polk and nCas9 are covalently linked sequentially in a polypeptide from N-terminus to C-terminus wherein the order is SV40 NLS-nCas9-linker-SV40 NLS-linker-polK (1-526, GE411-412RV)-linker-SV40NLS; and (b) at least two enhancers of template-based genome editing, comprising Vpx and FEN-1. In some embodiments, the system comprises (a) a polK polymerase and a SpyCas9 nickase (nCas9), wherein the polK and nCas9 are covalently linked sequentially in a polypeptide from N-terminus to C-terminus wherein the order is SV40 NLS-nCas9-linker-SV40 NLS-linker-polK (1-526, GE411-412RV)-linker-SV40NLS; and (b) a template guide RNA (tgRNA) comprising a first polynucleotide and a second polynucleotide, wherein (i) the first polynucleotide comprises a SpyCas9 guide RNA comprising, from 5′ to 3′, a guide sequence and the sequence of SEQ ID NO: 210, 212, 223, or 269; and (ii) the second polynucleotide is operably linked to the first polynucleotide and comprises a template sequence and a DNA-dependent DNA polymerase recruiting sequence (DRS). In some embodiments, the system comprises (a) a polK polymerase and a SpyCas9 nickase (nCas9), wherein the polK and nCas9 are covalently linked sequentially in a polypeptide from N-terminus to C-terminus wherein the order is SV40 NLS-nCas9-linker-SV40 NLS-linker-polK (1-526, GE411-412RV)-linker-SV40NLS; (b) a template guide RNA (tgRNA) comprising a first polynucleotide and a second polynucleotide, wherein (i) the first polynucleotide comprises a SpyCas9 guide RNA comprising, from 5′ to 3′, a guide sequence and the sequence of SEQ ID NO: 210, 212, 223, or 269; and (ii) the second polynucleotide is operably linked to the first polynucleotide and comprises a template sequence and a DNA-dependent DNA polymerase recruiting sequence (DRS); and (c) at least two enhancers of template-based genome editing, comprising Vpx and FEN-1. In some embodiments, the polypeptide comprises the sequence of SEQ ID NO: 1234. In some embodiments, the system further comprises a nicking guide RNA (ngRNA).

In some embodiments, the system comprises (a) a polK polymerase and a SpyCas9 nickase (nCas9), wherein the polk and nCas9 are covalently linked sequentially in a polypeptide from N-terminus to C-terminus wherein the order is SV40 NLS-nCas9-linker-SV40 NLS-linker-polK (1-526, GE411-412RV)-linker-SV40NLS; and (b) at least two enhancers of template-based genome editing, comprising Vpx and FEN-1. In some embodiments, the system comprises (a) a polK polymerase and a SpyCas9 nickase (nCas9), wherein the polK and nCas9 are covalently linked sequentially in a polypeptide from N-terminus to C-terminus wherein the order is SV40 NLS-nCas9-linker-SV40 NLS-linker-polK (1-526, GE411-412RV)-linker-SV40NLS; and (b) a template guide RNA (tgRNA) comprising a first polynucleotide and a second polynucleotide, wherein (i) the first polynucleotide comprises (a) a SpyCas9 guide RNA comprising, from 5′ to 3′, a guide sequence and the sequence of SEQ ID NO: 5, 27, 210, 221, 223, 269, 310, 326, 370, 201-208, 371, 373-374 and 384-400; (b) a SpyCas9 guide RNA comprising, from 5′ to 3′, a guide sequence and a nucleotide sequence that is at least 90%, 91%, 92%, 93%, 93%, 94%, 95%, 96%, 97%, 98%, 99%, or 100% of any one of SEQ ID NOs: 5, 27, 35, 210, 212, 223, 269,370, 201-208, 373-374, and 393-400; or (c) a SpyCas9 guide RNA comprising a nucleotide sequence that is at least 90%, 91%, 92%, 93%, 93%, 94%, 95%, 96%, 97%, 98%, 99%, or 100% of any one of SEQ ID NOs: 310, 312, 319-326, 347-349, 371, 384-392, 434, 438; and (ii) the second polynucleotide is operably linked to the first polynucleotide and comprises a template sequence and a DNA-dependent DNA polymerase recruiting sequence (DRS). In some embodiments, the system comprises (a) a polK polymerase and a SpyCas9 nickase (nCas9), wherein the polK and nCas9 are covalently linked sequentially in a polypeptide from N-terminus to C-terminus wherein the order is SV40 NLS-nCas9-linker-SV40 NLS-linker-polK (1-526, GE411-412RV)-linker-SV40NLS; (b) a template guide RNA (tgRNA) comprising a first polynucleotide and a second polynucleotide, wherein (i) the first polynucleotide comprises (a) a SpyCas9 guide RNA comprising, from 5′ to 3′, a guide sequence and the sequence of any one of SEQ ID NOs: 5, 27, 210, 212, 223, 269, 310, 326, 370, 201-208, 371, 373-374 or 384-400; (b) a SpyCas9 guide RNA comprising, from 5′ to 3′, a guide sequence and a nucleotide sequence that is at least 90%, 91%, 92%, 93%, 93%, 94%, 95%, 96%, 97%, 98%, 99%, or 100% of any one of SEQ ID NOs: 5, 27, 35, 210, 212, 223, 269, 370, 201-208, 373-374, and 393-400; (c) a SpyCas9 guide RNA comprising a nucleotide sequence that is at least 90%, 91%, 92%, 93%, 93%, 94%, 95%, 96%, 97%, 98%, 99%, or 100% of any one of SEQ ID NOs: 310, 312, 319-326, 347-349, 371, 384-392, 434, 438; and (ii) the second polynucleotide is operably linked to the first polynucleotide and comprises a template sequence and a DNA-dependent DNA polymerase recruiting sequence (DRS); and (c) at least two enhancers of template-based genome editing, comprising Vpx and FEN-1. In some embodiments, the polypeptide comprises the sequence of SEQ ID NO: 1234. In some embodiments, the system further comprises a nicking guide RNA (ngRNA).

In some embodiments, the polK and nCas9 are not covalently linked, and the polK is provided with a 3′ extension recruiting domain, wherein, the polk and the 3′ extension recruiting domain are covalently linked sequentially in a polypeptide from N-terminus to C-terminus in an order selected from: (a) 3′ extension recruiting domain-polK-NLS; (b) 3′ extension recruiting domain-linker-polK-linker-NLS; (c) 3′ extension recruiting domain-polK (GE411-412RV)-NLS; (d) 3′ extension recruiting domain-polK (GE411-412RV)-linker-NLS.

In some embodiments, wherein the DNA-dependent DNA polymerase is polK, the polypeptide comprises a peptide linker or further comprises an additional peptide linker. In some embodiments, the NLS is selected from c-Myc, SV40, nucleoplasmin, or snurportin-1 importin-β NLS, optionally wherein the NLS is an SV40 NLS.

In some embodiments, the polypeptide comprises a sequence selected from any one of SEQ ID NOs: 1024, 1027, 1030, 1036, 1039, 1042, 1045, 1048, 1053, 1056, 1059, 1074, 1200, 1202, 1229, 1234, and 1268, 1300, 1373, 1388, 1447, 1453, 1923, 1926, and 1929. In some embodiments, the polypeptide is encoded by a nucleotide sequence that has at least 90%, 91%, 92%, 93%, 94%, 95%, 96%, 97%, 98%, 99%, or 100% identity to any one of SEQ ID NOs: 1023, 1026, 1029, 1035, 1038, 1041, 1044, 1047, 1052, 1055, 1058, 1073, 1199, 1201, 1228, 1229, 1233, and 1267, 1299, 1372, 1387, 1446, 1452, 1922, 1925, or 1928. In some embodiments, the polypeptide comprises a sequence selected from any one of SEQ ID NOs: 1062 and 1067. In some embodiments, the polypeptide is encoded by a nucleotide sequence that has at least 90%, 91%, 92%, 93%, 94%, 95%, 96%, 97%, 98%, 99%, or 100% identity to any one of SEQ ID NOs: 1061 and 1066.

In some embodiments, the system comprises a T5 DNA polymerase (T5 pol). In some embodiments, the T5 pol comprises an amino acid sequence that has at least 90%, 91%, 92%, 93%, 94%, 95%, 96%, 97%, 98%, 99%, or 100% identity to any one of SEQ ID NO: 1116 or 1121. In some embodiments, the T5 pol comprises a mutation at D164 or E166 relative to SEQ ID NO: 1116. In some embodiments, the T5 pol comprises D164A and E166A mutations relative to SEQ ID NO: 1116. In some embodiments, the T5 pol comprises an amino acid sequence comprising an N-terminal deletion of 30 amino acids relative to SEQ ID NO: 1116. In some embodiments, the T5 pol is encoded by a nucleotide sequence that has at least 90%, 91%, 92%, 93%, 94%, 95%, 96%, 97%, 98%, 99%, or 100% identity to any one of SEQ ID NOs: 1115 or 1120.

In some embodiments, wherein the DNA-dependent DNA polymerase is T5, the T5 pol or the nCas9 is linked to a heterologous nuclear localization signal (NLS). In some embodiments, the T5 pol is linked to an MS2 coat protein (MCP domain). In some embodiments, the T5 pol is covalently linked to the nCas9. In some embodiments, the T5 pol is covalently linked to the nCas9 in sequence from N-terminus to C-terminus, nCas9-T5 pol. In some embodiments, the T5 pol and nCas9 are covalently linked with a peptide linker.

In some embodiments, the T5 pol and nCas9 are covalently linked sequentially in a polypeptide from N-terminus to C-terminus in an order selected from: (a) nCas9-T5Pol-NLS; (b) nCas9-linker-T5Pol-linker-NLS; (c) T5Pol-nCas9-NLS; (d) T5Pol-linker-nCas9-linker-NLS; (e) NLS-nCas9-NLS-T5Pol-binding site-NLS-NLS; (f) NLS-nCas9-linker-NLS-linker-T5Pol-linker-MCP-linker-NLS-linker-NLS; (g) nCas9-linker-T5Pol-linker-NLS; (h) nCas9-T5Pol(N30del)-NLS; (i) nCas9-linker-T5Pol(N30del)-linker-NLS; (j) nCas9-T5Pol(DA593R)-NLS; (k) nCas9-linker-T5Pol (A593R)-linker-NLS; (1) NLS-nCas9-NLS-T5Pol-NLS-NLS; (m) NLS-nCas9-linker-NLS-linker-T5Pol-linker-NLS-linker-NLS; (n) nCas9-NLS-T5Pol; (o) nCas9-linker-NLS-linker-T5Pol; (p) NLS-nCas9-NLS-T5Pol-NLS-NLS; (q) NLS-nCas9-linker-NLS-linker-T5Pol-linker-NLS-NLS; (r) nCas9-T5Pol (K425I)-NLS; or (s) nCas9-linker-T5Pol (K425I)-linker-NLS; (t) nCas9-T5Pol-NLS-heterodimerization domain, optionally wherein the heterodimerization domain comprises a coiled coil heterodimerization domain, optionally an EI domain or a KI domain; or (u) nCas9-linker-T5Pol-linker-NLS-linker-heterodimerization domain, optionally wherein the heterodimerization domain comprises a coiled coil heterodimerization domain, optionally an EI domain or a KI domain, optionally, wherein the T5pol comprises a mutation at D164 or E166 relative to SEQ ID NO: 1116, optionally wherein the T5 pol comprises D164A and E166A mutations relative to SEQ ID NO: 1116. In some embodiments, the T5 pol and nCas9 are covalently linked sequentially in a polypeptide from N-terminus to C-terminus in order wherein the order is NLS-nCas9-linker-NLS-linker-T5Pol-linker-NLS-NLS; optionally, wherein the T5pol comprises a mutation at D164 or E166 relative to SEQ ID NO: 1116, optionally wherein the T5 pol comprises D164A and E166A mutations relative to SEQ ID NO: 1116. In some embodiments, wherein the T5Pol and nCas9 are covalently linked sequentially in a polypeptide from N-terminus to C-terminus, the order is NLS-nCas9-linker-NLS-linker-T5Pol-linker-NLS-NLS-linker, wherein the T5Pol comprises a mutation at D164 or E166 relative to SEQ ID NO: 1116. In some embodiments, wherein the T5Pol and nCas9 are covalently linked sequentially in a polypeptide from N-terminus to C-terminus, the order is (a) NLS-nCas9-linker-NLS-linker-T5Pol-linker-NLS-NLS-linker-EI domain; or (b) b) NLS-nCas9-linker-NLS-linker-T5Pol-linker-NLS-NLS-linker-KI domain; wherein the T5Pol comprises a mutation at D164 or E166 relative to SEQ ID NO: 1116. In some embodiments, wherein the T5Pol and nCas9 are covalently linked sequentially in a polypeptide from N-terminus to C-terminus, the order is: (a) nucleoplasmin-NLS-nCas9-linker-SV40 NLS-linker-T5Pol-linker-SV40 NLS-c-Myc NLS; (b) nucleoplasmin NLS-nCas9-linker-SV40 NLS-linker-T5Pol-linker-SV40 NLS-c-Myc NLS-EI domain; or (c) nucleoplasmin NLS-nCas9-linker-SV40 NLS-linker-T5Pol-linker-SV40 NLS-c-Myc NLS-KI domain; wherein the T5Pol comprises a mutation at D164 or E166 relative to SEQ ID NO: 1116.

In some embodiments, the system comprises (a) a T5Pol polymerase and a SpyCas9 nickase (nCas9), wherein the T5Pol and nCas9 are covalently linked sequentially in a polypeptide from N-terminus to C-terminus wherein the order is nucleoplasmin NLS-nCas9-linker-SV40 NLS-linker-T5Pol-linker-SV40 NLS-c-Myc NLS; and (b) at least two enhancers of template-based genome editing, comprising Vpx and FEN-1. In some embodiments, the system comprises (a) a T5Pol polymerase and a SpyCas9 nickase (nCas9), wherein the T5Pol and nCas9 are covalently linked sequentially in a polypeptide from N-terminus to C-terminus wherein the order is nucleoplasmin NLS-nCas9-linker-SV40 NLS-linker-T5Pol-linker-SV40 NLS-c-Myc NLS; and (b) a template guide RNA (tgRNA) comprising a first polynucleotide and a second polynucleotide, wherein: (i) the first polynucleotide comprises (a) a SpyCas9 guide RNA comprising, from 5′ to 3′, a guide sequence and the sequence of any one of SEQ ID NOs: 5, 27, 210, 212, 223, 269, 310, 326, 370, 201-208, 371, 373-374, and 384-400; (b) a SpyCas9 guide RNA comprising, from 5′ to 3′, a guide sequence and a nucleotide sequence that is at least 90%, 91%, 92%, 93%, 93%, 94%, 95%, 96%, 97%, 98%, 99%, or 100% of any one of SEQ ID NOs: 5, 27, 35, 210, 212, 223, 269,370, 201-208, 373-374, and 393-400; or (c) a SpyCas9 guide RNA comprising a nucleotide sequence that is at least 90%, 91%, 92%, 93%, 93%, 94%, 95%, 96%, 97%, 98%, 99%, or 100% of any one of SEQ ID NOs: 310, 312, 319-326, 347-349, 371, 384-392, 434, 438; and (ii) the second polynucleotide is operably linked to the first polynucleotide and comprises a template sequence and a DNA-dependent DNA polymerase recruiting sequence (DRS). In some embodiments, the system comprises (a) a T5Pol polymerase and a SpyCas9 nickase (nCas9), wherein the T5Pol and nCas9 are covalently linked sequentially in a polypeptide from N-terminus to C-terminus wherein the order is nucleoplasmin NLS-nCas9-linker-SV40 NLS-linker-T5Pol-linker-SV40 NLS-c-Myc NLS; (b) a template guide RNA (tgRNA) comprising a first polynucleotide and a second polynucleotide, wherein: (i) the first polynucleotide comprises (a) a SpyCas9 guide RNA comprising, from 5′ to 3′, a guide sequence and the sequence of any one of SEQ ID NOs: 5, 27, 210, 212, 223, 269, 310, 326, 370, 201-208, 371, 373-374 and 384-400; (b) a SpyCas9 guide RNA comprising, from 5′ to 3′, a guide sequence and a nucleotide sequence that is at least 90%, 91%, 92%, 93%, 93%, 94%, 95%, 96%, 97%, 98%, 99%, or 100% of any one of SEQ ID NOs: 5, 27, 35, 210, 212, 223, 269, 370, 201-208, 373-374, and 393-400; or (c) a SpyCas9 guide RNA comprising a nucleotide sequence that is at least 90%, 91%, 92%, 93%, 93%, 94%, 95%, 96%, 97%, 98%, 99%, or 100% of any one of SEQ ID NOs: 310, 312, 319-326, 347-349, 371, 384-392, 434, 438; and (ii) the second polynucleotide is operably linked to the first polynucleotide and comprises a template sequence and a DNA-dependent DNA polymerase recruiting sequence (DRS); and (c) at least two enhancers of template-based genome editing, comprising Vpx and FEN-1. In some embodiments, the polypeptide comprises the sequence of SEQ ID NO: 1219. In some embodiments, the system further comprises a nicking guide RNA (ngRNA).

In some embodiments, the T5 pol and the nCas9 are not covalently linked, and the T5 pol is provided with a 3′ extension-recruiting domain, wherein, the T5 pol and the 3′ extension-recruiting domain are covalently linked sequentially in a polypeptide from N-terminus to C-terminus in an order selected from: (a) 3′ extension-recruiting domain-T5Pol(D164A,E166A)-NLS; or (b) 3′ extension-recruiting domain-linker-T5Pol(D164A,E166A)-linker-NLS.

In some embodiments, wherein the DNA-dependent DNA polymerase comprises T5 pol, the polypeptide comprises a peptide linker or further comprises an additional peptide linker. In some embodiments, the NLS is selected from a c-Myc NLS, an SV40 NLS, a nucleoplasmin NLS, or a snurportin-1 importin-β NLS, optionally wherein each NLS is independently selected from an SV40 NLS, a nucleoplasmin NLS, or a c-Myc NLS.

In some embodiments, wherein the DNA-dependent DNA polymerase comprises T5 pol, the polypeptide comprises a heterodimerization domain, optionally a self-assembling coiled coil heterodimerization domain. In some embodiments, the heterodimerization domain is selected from KI, EI, KV, and EV. In some embodiments, the heterodimerization domain is KI or EI. In some embodiments, the heterodimerization domain is KI. In some embodiments, the heterodimerization domain is EI.

In some embodiments, the polypeptide comprises a sequence selected from any one of SEQ ID NO: 1124, 1127, 1129, 1132, 1134, 1137, 1140, 1142, 1145, 1147, 1150, 1152, 1155, 1157, 1160, 1162, 1165, 1167, 1170, 1172, 1216, 1219, 1224, 1254, and 1259, 1276, 1303, 1307, 1376, 1450, 1932, 1935, and 1938. In some embodiments, the polypeptide comprises a sequence selected from any one of SEQ ID NO: 1124, 1129, 1134, 1137, 1142, 1147, 1152, 1157, 1162, 1167, 1172, 1219, 1224, 1254, and 1259, 1276, 1307, 1932, 1935, and 1938. In some embodiments, the polypeptide is encoded by a nucleotide sequence that has at least 90%, 91%, 92%, 93%, 94%, 95%, 96%, 97%, 98%, 99%, or 100% identity to any one of SEQ ID NOs: 1123, 1126, 1128, 1131, 1133, 1136, 1139, 1141, 1144, 1146, 1149, 1151, 1154, 1156, 1159, 1161, 1164, 1166, 1169, 1171, 1215, 1218, 1223, 1253, and 1258, 1275, 1302, 1306, 1375, 1449, 1931, 1934, and 1937. In some embodiments, the polypeptide is encoded by a nucleotide sequence that has at least 90%, 91%, 92%, 93%, 94%, 95%, 96%, 97%, 98%, 99%, or 100% identity to any one of SEQ ID NOs: 1123, 1128, 1133, 1136, 1141, 1146, 1151, 1156, 1161, 1166, 1171, 1218, 1223, 1253, and 1258, 1275, 1306, 1931, 1934, and 1937. In some embodiments, the polypeptide comprises a sequence selected from any one of SEQ ID NO: 1175, 1177, 1180, and 1182. In some embodiments, the polypeptide comprises a sequence selected from SEQ ID NO: 1177 and 1182. In some embodiments, the polypeptide is encoded by a nucleotide sequence that has at least 90%, 91%, 92%, 93%, 94%, 95%, 96%, 97%, 98%, 99%, or 100% identity to any one of SEQ ID NOs: 1174, 1176, 1179, and 1181. In some embodiments, the polypeptide is encoded by a nucleotide sequence that has at least 90%, 91%, 92%, 93%, 94%, 95%, 96%, 97%, 98%, 99%, or 100% identity to SEQ ID NOs: 1176 or 1181. It is to be understood that, if the sequence includes a HiBiT tag, the tag is not included in the amino acid sequence within the scope of the claimed invention.

Also provided herein are systems for template-based genome editing. In some embodiments, the system comprises (a) a first polynucleotide comprising a SpyCas9 guide RNA comprising (i) a spacer that is complementary to a target region in a target strand of a DNA duplex target nucleic acid; and (ii) a scaffold; (b) a second polynucleotide operably linked to the first polynucleotide, comprising: (iii) a template sequence; and (iv) a DNA-dependent DNA polymerase recruiting sequence (DRS) 3′ of the template sequence, further wherein the DRS comprises a sequence that is complementary to at least 8 nucleotides of a DRS-complementary region of a non-target strand of the DNA duplex target nucleic acid; (c) a SpyCas9 nickase; and (d) a DNA-dependent DNA polymerase operably linked to the SpyCas9 nickase. In some embodiments, the DRS comprises RNA nucleotides. In some embodiments, the DRS of the tgRNA comprises DNA nucleotides. In some embodiments, the DRS of the tgRNA comprises both RNA nucleotide(s) and DNA nucleotide(s).

In some embodiments, the system comprises (a) a first polynucleotide comprising a SpyCas9 guide RNA comprising (i) a spacer that is complementary to a target region in a target strand of a DNA duplex target nucleic acid; and (ii) a scaffold; (b) a second polynucleotide operably linked to the first polynucleotide, comprising: (iii) a template sequence; and (iv) a DNA-dependent DNA polymerase recruiting sequence (DRS) 3′ of the template sequence, further wherein the DRS comprises a sequence that is complementary to at least 8 nucleotides of a DRS-complementary region of a non-target strand of the DNA duplex target nucleic acid, wherein the DRS comprises RNA nucleotides; (c) a SpyCas9 nickase; and (d) a DNA-dependent DNA polymerase operably linked to the SpyCas9 nickase.

In some embodiments, the system further comprises a nicking guide RNA (ngRNA).

In some embodiments, the ngRNA comprises a spacer that hybridizes to a sequence such that the nick site of the ngRNA is within 200 nucleotides 5′ or 3′ from the nick site of the template guide RNA and on the same strand as the first region. In some embodiments, the nick site of the ngRNA is about 20-200 nucleotides 5′ or 3′ from the nick site of the template guide RNA and on the same strand as the first region, that is the target strand of the DNA duplex target nucleic acid, i.e., the strand to which the spacer of the tgRNA hybridizes. In some embodiments, the nick site of the ngRNA is within the first region. In some embodiments, the nick site of the ngRNA is outside the first region.

Provided herein are also fusion polypeptides for template-based genome editing.

In some embodiments, the fusion polypeptide comprises a polK comprising GE411-412RV mutations relative to SEQ ID NO: 1021, a SpyCas9 nickase (nCas9), and a heterologous nuclear localization signal (NLS). In some embodiments, the polK comprises amino acid sequence that has at least 90%, 91%, 92%, 93%, 94%, 95%, 96%, 97%, 98%, 99%, or 100% identity to amino acid residues 19-526 of SEQ ID NO: 1021; or an amino acid sequence that has at least 90%, 91%, 92%, 93%, 94%, 95%, 96%, 97%, 98%, 99%, or 100% identity to SEQ ID NO: 1021 or 1206. In some embodiments, the polK comprises an amino acid sequence of residues 19-526 of SEQ ID NO: 1021. In some embodiments, the polK comprises an amino acid sequence of SEQ ID NO: 1206. In some embodiments, the polk is encoded by a nucleotide sequence that has at least 90%, 91%, 92%, 93%, 94%, 95%, 96%, 97%, 98%, 99%, or 100% identity to any one of SEQ ID NOs: 1020 or 1205. In some embodiments, the nCas9 comprises an amino acid sequence that has at least 90%, 91%, 92%, 93%, 94%, 95%, 96%, 97%, 98%, 99%, or 100% identity to any one of SEQ ID NOs: 972, 974, 976, 982, 984, 986, 988, 990, 992, 994, 996, 998, 1000, and 1014. In some embodiments, the nCas9 comprises an amino acid sequence of any one of SEQ ID NOs: 972, 974, 976, 982, 984, 986, 988, 990, 992, 994, 996, 998, 1000, and 1014. In some embodiments, the nCas9 is encoded by a nucleotide sequence that has at least 90%, 91%, 92%, 93%, 94%, 95%, 96%, 97%, 98%, 99%, or 100% identity to any one of SEQ ID NOs: 971, 973, 975, 981, 983, 985, 987, 989, 991, 993, 995, 997, 999, and 1013.

In some embodiments, the fusion polypeptide comprises a human polK comprising GE411-412RV mutations relative to SEQ ID NO: 1021, a SpyCas9 nickase (nCas9), and a heterologous nuclear localization signal (NLS). In some embodiments, the polK comprises amino acid sequence that has at least 90%, 91%, 92%, 93%, 94%, 95%, 96%, 97%, 98%, 99%, or 100% identity to amino acid residues 19-526 of SEQ ID NO: 1021; or an amino acid sequence that has at least 90%, 91%, 92%, 93%, 94%, 95%, 96%, 97%, 98%, 99%, or 100% identity to SEQ ID NO: 1021. In some embodiments, the polK comprises an amino acid sequence of residues 19-526 of SEQ ID NO: 1021. In some embodiments, the polK is encoded by a nucleotide sequence that has at least 90%, 91%, 92%, 93%, 94%, 95%, 96%, 97%, 98%, 99%, or 100% identity to any one of SEQ ID NOs: 1020 or 1205. In some embodiments, the nCas9 comprises an amino acid sequence that has at least 90%, 91%, 92%, 93%, 94%, 95%, 96%, 97%, 98%, 99%, or 100% identity to any one of SEQ ID NOs: 1014, 972, 974, 976, 982, 984, 986, 988, 990, 992, 994, 996, 998, and 1000. In some embodiments, the nCas9 comprises an amino acid sequence of any one of SEQ ID NOs: 1014, 972, 974, 976, 982, 984, 986, 988, 990, 992, 994, 996, 998, and 1000. In some embodiments, the nCas9 is encoded by a nucleotide sequence that has at least 90%, 91%, 92%, 93%, 94%, 95%, 96%, 97%, 98%, 99%, or 100% identity to any one of SEQ ID NOs: 1013, 971, 973, 975, 981, 983, 985, 987, 989, 991, 993, 995, 997, and 999.

In some embodiments, the fusion polypeptide comprises a cynomolgus polK comprising GE411-412RV mutations relative to SEQ ID NO: 1261, a SpyCas9 nickase (nCas9), and a heterologous nuclear localization signal (NLS). In some embodiments, the polK comprises amino acid sequence that has at least 90%, 91%, 92%, 93%, 94%, 95%, 96%, 97%, 98%, 99%, or 100% identity to amino acid residues 19-526 of SEQ ID NO: 1261; or an amino acid sequence that has at least 90%, 91%, 92%, 93%, 94%, 95%, 96%, 97%, 98%, 99%, or 100% identity to SEQ ID NO: 1261. In some embodiments, the polk comprises an amino acid sequence of residues 19-526 of SEQ ID NO: 1261. In some embodiments, the polK is encoded by a nucleotide sequence that has at least 90%, 91%, 92%, 93%, 94%, 95%, 96%, 97%, 98%, 99%, or 100% identity to any one of SEQ ID NO: 1260. In some embodiments, the nCas9 comprises an amino acid sequence that has at least 90%, 91%, 92%, 93%, 94%, 95%, 96%, 97%, 98%, 99%, or 100% identity to any one of SEQ ID NOs: 1014, 972, 974, 976, 982, 984, 986, 988, 990, 992, 994, 996, 998, and 1000. In some embodiments, the nCas9 comprises an amino acid sequence of any one of SEQ ID NOs: 1014, 972, 974, 976, 982, 984, 986, 988, 990, 992, 994, 996, 998, and 1000. In some embodiments, the nCas9 is encoded by a nucleotide sequence that has at least 90%, 91%, 92%, 93%, 94%, 95%, 96%, 97%, 98%, 99%, or 100% identity to any one of SEQ ID NOs: 1013, 971, 973, 975, 981, 983, 985, 987, 989, 991, 993, 995, 997, and 999. In some embodiments, a nucleic acid (e.g., mRNA) comprising an open reading frame (ORF) encoding the aforementioned fusion polypeptide comprising cynomolgus polK comprising GE411-412RV mutations relative to SEQ ID NO: 1261, a SpyCas9 nickase (nCas9), and a heterologous nuclear localization signal (NLS), is provided wherein the nucleic acid comprises a sequence encoding a nucleotide sequence that has at least 90%, 91%, 92%, 93%, 94%, 95%, 96%, 97%, 98%, 99%, or 100% identity to any one of SEQ ID NO: 1260

In some embodiments, the fusion polypeptide comprises a mouse polK comprising GE410-411RV mutations relative to SEQ ID NO: 1263, a SpyCas9 nickase (nCas9), and a heterologous nuclear localization signal (NLS). In some embodiments, the polK comprises amino acid sequence that has at least 90%, 91%, 92%, 93%, 94%, 95%, 96%, 97%, 98%, 99%, or 100% identity to amino acid residues 19-526 of SEQ ID NO: 1263; or an amino acid sequence that has at least 90%, 91%, 92%, 93%, 94%, 95%, 96%, 97%, 98%, 99%, or 100% identity to SEQ ID NO: 1263. In some embodiments, the polk comprises an amino acid sequence of residues 19-526 of SEQ ID NO: 1263. In some embodiments, the polK is encoded by a nucleotide sequence that has at least 90%, 91%, 92%, 93%, 94%, 95%, 96%, 97%, 98%, 99%, or 100% identity to any one of SEQ ID NO: 1262. In some embodiments, the nCas9 comprises an amino acid sequence that has at least 90%, 91%, 92%, 93%, 94%, 95%, 96%, 97%, 98%, 99%, or 100% identity to any one of SEQ ID NOs: 1014, 972, 974, 976, 982, 984, 986, 988, 990, 992, 994, 996, 998, and 1000. In some embodiments, the nCas9 comprises an amino acid sequence of any one of SEQ ID NOs: 1014, 972, 974, 976, 982, 984, 986, 988, 990, 992, 994, 996, 998, and 1000. In some embodiments, the nCas9 is encoded by a nucleotide sequence that has at least 90%, 91%, 92%, 93%, 94%, 95%, 96%, 97%, 98%, 99%, or 100% identity to any one of SEQ ID NOs: 1013, 971, 973, 975, 981, 983, 985, 987, 989, 991, 993, 995, 997, and 999. In some embodiments, a nucleic acid (e.g., mRNA) comprising an open reading frame (ORF) encoding the aforementioned fusion polypeptide comprising a mouse polk comprising GE410-411RV mutations relative to SEQ ID NO: 1263, a SpyCas9 nickase (nCas9), and a heterologous nuclear localization signal (NLS) is provided.

In some embodiments, the polK and nCas9 are in a sequence from N-terminus to C-terminus, polk-nCas9. In some embodiments, the polk and nCas9 are in a sequence from N-terminus to C-terminus, nCas9-polK.

In some embodiments, the fusion protein further comprises a peptide linker. In some embodiments, the polK and nCas9 are covalently linked in sequence from N-terminus to C-terminus selected from (a) NLS-nCas9-NLS-polK; (b) NLS-nCas9-linker-NLS-linker-polK; (c) polK-NLS-nCas9-NLS; (d) polK-linker-NLS-linker-nCas9-linker-NLS; (e) NLS-nCas9-NLS-polK (GE411-412RV); (f) NLS-nCas9-linker-NLS-polK (GE411-412RV); (g) NLS-nCas9-NLS-polK (19-526); (h) NLS-nCas9-linker-NLS-linker-polK (19-526); (i) polK (19-526)-NLS-nCas9-NLS; (j) polK (19-526)-Linker-NLS-linker-nCas9-linker-NLS; (k) polK (GE411-412RV)-NLS-NLS-nCas9; (l) polK (GE411-412RV)-linker-NLS-linker-NLS-nCas9; (m) NLS-nCas9-NLS-polK (19-526, GE411-412RV); (n) NLS-nCas9-linker-NLS-linker-polK (19-526, GE411-412RV); (o) NLS-nCas9-NLS-polK-NLS; (p) NLS-nCas9-linker-NLS-linker-polK-linker-NLS; (q) NLS-nCas9-NLS-polK (1-526, GE411-412RV)-NLS; (r) NLS-nCas9-linker-NLS-linker-polK (1-526, GE411-412RV)-linker-NLS; or (s) nCas9-polK (1-526); (t) polK (1-526, GE411-412RV)-nCas9-NLS-heterodimerization domain, optionally wherein the heterodimerization domain comprises a coiled coil heterodimerization domain, optionally an EI domain or a KI domain; (u) polK (1-526, GE411-412RV)-linker-NLS-nCas9-linker-NLS-heterodimerization domain, optionally wherein the heterodimerization domain comprises a coiled coil heterodimerization domain, optionally an EI domain or a KI domain, optionally wherein the order is NLS-nCas9-linker-NLS-linker-polK (1-526, GE411-412RV)-linker-NLS, optionally further comprising a heterodimerization domain comprising a coiled coil heterodimerization domain, optionally an EI domain or a KI domain. In some embodiments, the fusion protein comprises a peptide linker or further comprises an additional peptide linker. In some embodiments, the NLS is selected from c-Myc, SV40, nucleoplasmin, or snurportin-1 importin-β NLS, optionally wherein the NLS is an SV40 NLS.

In some embodiments, the fusion polypeptide comprises a sequence selected from any one of SEQ ID NOs: 1024, 1027, 1030, 1036, 1039, 1042, 1045, 1048, 1053, 1056, 1059, 1074, 1200, 1202, 1229, 1234, and 1268, 1300, 1373, 1388, 1447, 1453, 1923, 1926, and 1929. In some embodiments, the polypeptide is encoded by a nucleotide sequence that has at least 90%, 91%, 92%, 93%, 94%, 95%, 96%, 97%, 98%, 99%, or 100% identity to any one of SEQ ID NOs: 1023, 1026, 1029, 1035, 1038, 1041, 1044, 1047, 1052, 1055, 1058, 1073, 1199, 1201, 1228, 1233, and 1267, 1299, 1372, 1387, 1446, 1452, 1922, 1925, and 1928. In some embodiments, a nucleic acid (e.g., mRNA) comprising an open reading frame (ORF) encoding the aforementioned fusion polypeptide is provided, wherein the nucleic acid comprising a nucleotide sequence that has at least 90%, 91%, 92%, 93%, 94%, 95%, 96%, 97%, 98%, 99%, or 100% identity to any one of SEQ ID NOs: 1023, 1026, 1029, 1035, 1038, 1041, 1044, 1047, 1052, 1055, 1058, 1073, 1199, 1201, 1228, 1233, 1267, and 1299, 1372, 1387, 1446, 1452, 1922, 1925, and 1928.

In some embodiments, the fusion polypeptide comprises a T5 pol comprising D164A and E166A mutations relative to SEQ ID NO: 1021 or 1116, a SpyCas9 nickase (nCas9), and a heterologous nuclear localization signal (NLS). In some embodiments, the fusion polypeptide comprises a T5 pol comprising D164A and E166A mutations relative to SEQ ID NO: 1116, a SpyCas9 nickase (nCas9), and a heterologous nuclear localization signal (NLS). In some embodiments, the T5 pol comprises an amino acid sequence that has at least 90%, 91%, 92%, 93%, 94%, 95%, 96%, 97%, 98%, 99%, or 100% identity to any one of SEQ ID NO: 1116 or 1121. In some embodiments, the T5 pol comprises an N-terminal deletion of 30 amino acids relative to SEQ ID NO: 1116. In some embodiments, the T5 pol is encoded by a nucleotide sequence that has at least 90%, 91%, 92%, 93%, 94%, 95%, 96%, 97%, 98%, 99%, or 100% identity to any one of SEQ ID NOs: 1115 or 1120. In some embodiments, the nCas9 comprises an amino acid sequence that has at least 90%, 91%, 92%, 93%, 94%, 95%, 96%, 97%, 98%, 99%, or 100% identity to any one of SEQ ID NOs: 972, 974, 976, 982, 984, 986, 988, 990, 992, 994, 996, 998, 1000, and 1014. In some embodiments, the nCas9 comprises an amino acid sequence of any one of SEQ ID NOs: 972, 974, 976, 982, 982, 984, 986, 988, 990, 992, 994, 996, 998, 1000, and 1014. In some embodiments, the nCas9 is encoded by a nucleotide sequence that has at least 90%, 91%, 92%, 93%, 94%, 95%, 96%, 97%, 98%, 99%, or 100% identity to any one of SEQ ID NOs: 971, 973, 975, 981, 983, 985, 987, 989, 991, 993, 995, 997, 999, and 1013. In some embodiments, a nucleic acid (e.g., mRNA) comprising an open reading frame (ORF) encoding the aforementioned fusion protein is provided.

In some embodiments, the T5 pol and the nCas9 are in a sequence from N-terminus to C-terminus, nCas9-T5 pol. In some embodiments, the fusion protein further comprises a peptide linker.

In some embodiments, the polk and nCas9 are covalently linked in sequence from (a)N-terminus to C-terminus selected from a) nCas9-T5Pol-NLS (b) nCas9-linker-T5Pol-linker-NLS; (c) T5Pol-nCas9-NLS; (d) T5Pol-linker-nCas9-linker-NLS; (e) NLS-nCas9-NLS-T5Pol-binding site-NLS-NLS; (f) NLS-nCas9-linker-NLS-linker-T5Pol-linker-MCP-linker-NLS-linker-NLS; (g) nCas9-linker-T5Pol-linker-NLS; (h) nCas9-T5Pol(N30del,)-NLS; (i) nCas9-linker-T5Pol(N30del)-linker-NLS; (j) nCas9-T5Pol (A593R)-NLS; (k) nCas9-linker-T5Pol(DA593R)-linker-NLS; (l) NLS-nCas9-NLS-T5Pol-NLS-NLS; (m) NLS-nCas9-linker-NLS-linker-T5Pol-linker-NLS-linker-NLS; (n) nCas9-NLS-T5Pol; (o) nCas9-linker-NLS-linker-T5Pol; (p) NLS-nCas9-NLS-T5Pol-NLS-NLS; (q) NLS-nCas9-linker-NLS-linker-T5Pol-linker-NLS-NLS; (r) nCas9-T5Pol (K425I)-NLS; or (s) nCas9-linker-T5Pol (K425I)-linker-NLS; (t) nCas9-T5Pol-NLS-heterodimerization domain, optionally wherein the heterodimerization domain comprises a coiled coil peptide, optionally an EI domain or a KI domain; or (u) nCas9-linker-T5Pol-linker-NLS-linker-heterodimerization domain, optionally wherein the heterodimerization domain comprises a coiled coil heterodimerization domain, optionally an EI domain or a KI domain, optionally, wherein the T5pol comprises a mutation at D164 or E166 relative to SEQ ID NO: 1116, optionally wherein the T5 pol comprises D164A and E166A mutations relative to SEQ ID NO: 1116. In some embodiments, a nucleic acid (e.g., mRNA) comprising an open reading frame (ORF) encoding the aforementioned fusion polypeptide is provided.

In some embodiments, the T5 pol and nCas9 are covalently linked sequentially in a polypeptide from N-terminus to C-terminus in order wherein the order is NLS-nCas9-linker-NLS-linker-T5Pol-linker-NLS-NLS; optionally, wherein the T5pol comprises a mutation at D164 or E166 relative to SEQ ID NO: 1116, optionally wherein the T5 pol comprises D164A and E166A mutations relative to SEQ ID NO: 1116. In some embodiments, the polypeptide comprises a linker or further comprises an additional linker. In some embodiments, the NLS is selected from a c-Myc NLS, an SV40 NLS, a nucleoplasmin NLS, or a snurportin-1 importin-β NLS, optionally wherein the NLS is selected from c-Myc NLS, an SV40 NLS, or a nucleoplasmin NLS. In some embodiments, a nucleic acid (e.g., mRNA) comprising an open reading frame (ORF) encoding the aforementioned fusion polypeptide is provided.

In some embodiments, the DNA-dependent DNA polymerase polypeptide comprises a heterodimerization domain, optionally a self-assembling coiled coil heterodimerization domain. In some embodiments, the heterodimerization domain is selected from KI, EI, KV, and EV. In some embodiments, the heterodimerization domain is KI or EI. In some embodiments, the heterodimerization domain is KI. In some embodiments, the heterodimerization domain is EI.

In some embodiments, the fusion polypeptide comprises a sequence selected from any one of SEQ ID NO: 1124, 1127, 1129, 1132, 1134, 1137, 1140, 1142, 1145, 1147, 1150, 1152, 1155, 1157, 1160, 1162, 1165, 1167, 1170, 1172, 1216, 1219, 1224, 1254, and 1259, 1276, 1303, 1307, 1376, 1450, 1932, 1935, and 1938. In some embodiments, the polypeptide comprises a sequence selected from any one of SEQ ID NO: 1124, 1129, 1134, 1137, 1142, 1147, 1152, 1157, 1162, 1167, 1172, 1219, 1224, 1254, and 1259, 1276, 1307, 1932, 1935, and 1938. In some embodiments the polypeptide is encoded by a nucleotide sequence that has at least 90%, 91%, 92%, 93%, 94%, 95%, 96%, 97%, 98%, 99%, or 100% identity to any one of SEQ ID NOs: 1123, 1126, 1128, 1131, 1133, 1136, 1139, 1144, 1146, 1149, 1151, 1154, 1156, 1159, 1161, 1164, 1166, 1169, 1171, 1215, 1218, 1223, 1253, and 1258, 1275, 1302, 1306, 1375, 1449, 1931, 1934, and 1937. In some embodiments, the polypeptide is encoded by a nucleotide sequence that has at least 90%, 91%, 92%, 93%, 94%, 95%, 96%, 97%, 98%, 99%, or 100% identity to any one of SEQ ID NOs: 1123, 1128, 1133, 1136, 1141, 1146, 1151, 1156, 1161, 1166, 1171, 1218, 1223, 1253, and 1258, 1275, 1306, 1931, 1934, and 1937. In some embodiments, a nucleic acid (e.g., mRNA) comprising an open reading frame (ORF) encoding the aforementioned fusion polypeptide is provided, the nucleic acid comprising a nucleotide sequence that has at least 90%, 91%, 92%, 93%, 94%, 95%, 96%, 97%, 98%, 99%, or 100% identity to any one of SEQ ID NOs: 1123, 1126, 1128, 1131, 1133, 1136, 1139, 1144, 1146, 1149, 1151, 1154, 1156, 1159, 1161, 1164, 1166, 1169, 1171, 1215, 1218, 1223, 1253, and 1258, 1275, 1302, 1306, 1375, 1449, 1931, 1934, and 1937.

It is to be understood that, if the sequence includes a HiBiT tag, the tag is not included in the amino acid sequence within the scope of the claimed invention.

As used herein, “HiBiT” or “HiBiT tag” refers to a 11-amino acid tag sequence (e.g., SEQ ID NO: 1832) typically used in vitro for observing protein expression. A person of skill in the art would recognize that a HiBiT tag can be useful for research purposes, but that any polypeptide or fusion protein constructs described herein containing a tag, or polynucleotides encoding same, can also be used without the tag, without further optimization. It is to be understood that, if the sequence includes a tag, the tag is not included in the amino acid sequence within the scope of the claimed invention. It is to be understood that, if the amino acid sequence includes a tag (e.g., HiBiT tag or HiBiT tag with preceding linker (e.g., SEQ ID NO: 1833 or 1836), the tag (and preceding linker, if present) is not included in the amino acid sequence within the scope of the claimed invention. It is to be understood that, if the nucleic acid sequence includes a sequence encoding tag (e.g., HiBiT tag (e.g., SEQ ID NO: 1832 or 1834) or a HiBiT tag with preceding linker (e.g., SEQ ID NO: 1835), the sequence encoding the tag (and preceding linker) is not included in the nucleic acid sequence within the scope of the claimed invention.

### Compositions, Systems and Kits

Compositions, systems, and kits comprising (a) a SpyCas9 nickase or a nucleic acid encoding the same, (b) a DNA-dependent DNA polymerase or a nucleic acid encoding the same, and (c) a template guide RNA for template-based genome editing described herein and a carrier, excipient, diluent, or the like are encompassed. In some instances, the excipient or diluent is inert. In some instances, the excipient or diluent is not inert. In some instances, a pharmaceutical formulation is provided comprising (a) a SpyCas9 nickase or a nucleic acid encoding the same, (b) a DNA-dependent DNA polymerase or a nucleic acid encoding the same, and (c) a template guide RNA for template-based genome editing described herein and a pharmaceutically acceptable carrier, excipient, diluent, or the like. In some embodiments, the pharmaceutical formulation further comprises an LNP.

Also provided are compositions, systems, and kits comprising (a) a SpyCas9 nickase or a nucleic acid encoding the same, (b) a DNA-dependent DNA polymerase or a nucleic acid encoding the same, and (c) a template guide RNA for template-based genome editing, compositions, or pharmaceutical formulations described herein. In some embodiments, a kit further comprises a solvent, solution, buffer, each separate from the composition or pharmaceutical formulation, instructions, or desiccant.

Also provided are compositions, systems, kits, and pharmaceutical compositions comprising an enhancer of template-based genome editing. In some embodiments, the composition, system, kit, or pharmaceutical formulations comprises (a) a SpyCas9 nickase or a nucleic acid encoding the same, (b) a DNA-dependent DNA polymerase or a nucleic acid encoding the same, (c) a template guide RNA for template-based genome editing, and (d) one or more enhancers. In some embodiments, the enhancer is an inhibitor of deoxynucleotide triphosphohydrolase. In some embodiments, wherein the enhancer is an inhibitor of deoxynucleotide triphosphohydrolase, the deoxynucleotide triphosphohydrolase comprises SAM domain and HD domain-containing protein 1 (SAMHD1). In some embodiments, inhibiting SAMHD1 comprises contacting the SAMHD1 with a Vpx, BGLF4, M97, or KSHV ORF36 protein or expressing the Vpx, BGLF4, M97, or KSHV ORF36 protein in a cell. In some embodiments, inhibiting SAMHD1 comprises contacting an mRNA encoding the SAMHD1 with a microRNA, shRNA, or siRNA that hybridizes to the mRNA, or expressing the microRNA, shRNA, or siRNA in a cell. In some embodiments, inhibiting SAMHD1 comprises contacting the SAMHD1 with a small molecule SAMHD1 inhibitor.

As shown in FIG. 3D, the compositions and methods provided herein further may comprise one or more enhancers of template-based genome editing. In some embodiments, such enhancers may promote DNA synthesis by increasing the concentration of dNTPs within the cell, e.g., by inhibiting a deoxynucleotide triphosphohydrolase protein (see AF1 in FIG. 3D). In some embodiments, such enhancers may promote the hybridization of the newly synthesized DNA strand and the target strand. For example, DNA repair proteins may remove the unedited flap 3′ of the nick (see AF2 in FIG. 3D). Removal of the unedited flap 3′ of the nick may promote hybridization of the newly synthesized DNA strand to the target strand of the DNA duplex target nucleic acid.

In some embodiments, the enhancer is a Vpx protein, BGLF4 protein, M97 protein, KSHV ORF36 protein, or an MLP1 protein. In some embodiments, the enhancer is a Vpx protein. In some embodiments, the Vpx protein comprises an amino acid sequence that has at least 90%, 91%, 92%, 93%, 94%, 95%, 96%, 97%, 98%, 99%, or 100% identity to any one of SEQ ID NOs: 1464, 1469, and 1503-1505. In some embodiments, the Vpx protein is encoded by a nucleotide sequence that has at least 90%, 91%, 92%, 93%, 94%, 95%, 96%, 97%, 98%, 99%, or 100% identity to SEQ ID NO: 1462, 1463, 1468, 1470, 1499, 1501, 1502, 1521, or 1585, or 3530. In some embodiments, the Vpx comprises a mutation at P109 relative to SEQ ID NO: 1504. In some embodiments, the Vpx comprises a P109A mutation relative to SEQ ID NO: 1504. In some embodiments, the amino acid sequence of the Vpx protein lacks an N-terminal methionine, for instance, where the Vpx protein is comprised within a fusion protein. In some embodiments, the amino acid sequence of the Vpx protein lacks the N-terminal methionine of any one of SEQ ID NOs: 1464, 1469, and 1503-1505, for instance, where the Vpx protein is comprised within a fusion protein. In other embodiments, the amino acid sequence of the Vpx protein comprises any one of SEQ ID NOs: 1464, 1469, and 1503-1505, including or lacking the N-terminal methionine. In some embodiments, the nucleotide sequence of the Vpx protein lacks a start codon or stop codon. In some embodiments, the nucleotide sequence encoding the Vpx protein lacks the start codon or stop codon of any one of SEQ ID NOs: 1462, 1463, 1468, 1499, 1470, 1501, 1502, 1521, or 1585, or 3530, for instance, where the Vpx protein is comprised within a fusion protein. In other embodiments, the nucleotide sequence encoding the Vpx protein comprises any one of SEQ ID NOs: 1462, 1463, 1468, 1499, 1501, 1470, 1502, 1521, or 1585, or 3530, including or lacking the start codon or stop codon.

In some embodiments, the enhancer is a BGLF4 protein. In some embodiments, the BGLF4 comprises an amino acid sequence that has at least 90%, 91%, 92%, 93%, 94%, 95%, 96%, 97%, 98%, 99%, or 100% identity to SEQ ID NO: 1517 or 1518. In some embodiments, the BGLF4 protein is encoded by a nucleotide sequence that has at least 90%, 91%, 92%, 93%, 94%, 95%, 96%, 97%, 98%, 99%, or 100% identity to SEQ ID NO: 1522 or 1523. In some embodiments, the amino acid sequence of the BGLF4 protein lacks an N-terminal methionine. In some embodiments, the amino acid sequence of the BGLF4 protein lacks the N-terminal methionine of SEQ ID NO: 1517 or 1518, for instance, where the BGLF4 protein is comprised within a fusion protein. In other embodiments, the amino acid sequence of the BGLF4 protein comprises SEQ ID NO: 1517 or 1518, including or lacking the N-terminal methionine. In some embodiments, the nucleotide sequence of the BGLF4 protein lacks a start codon or stop codon. In some embodiments, the nucleotide sequence encoding the BGLF4 protein lacks the start codon or stop codon of SEQ ID NO: 1522 or 1523, for instance, where the BGLF4 protein is comprised within a fusion protein. In other embodiments, the nucleotide sequence encoding the BGLF4 protein comprises SEQ ID NO: 1522 or 1523, including or lacking the start codon or stop codon.

In some embodiments, the enhancer is an M97 protein. In some embodiments, the M97 protein comprises an amino acid sequence that has at least 90%, 91%, 92%, 93%, 94%, 95%, 96%, 97%, 98%, 99%, or 100% identity to SEQ ID NO: 1500. In some embodiments, the M97 protein is encoded by a nucleotide sequence that has at least 90%, 91%, 92%, 93%, 94%, 95%, 96%, 97%, 98%, 99%, or 100% identity to SEQ ID NO: 1499. In some embodiments, the amino acid sequence of the M97 protein lacks an N-terminal methionine. In some embodiments, the amino acid sequence of the M97 protein lacks the N-terminal methionine of SEQ ID NO: 1500, for instance, where the M97 protein is comprised within a fusion protein. In other embodiments, the amino acid sequence of the M97 protein comprises SEQ ID NO: 1500, including or lacking the N-terminal methionine. In some embodiments, the nucleotide sequence encoding the M97 protein lacks a start codon or stop codon. In some embodiments, the nucleotide sequence encoding the M97 protein lacks the start codon or stop codon of SEQ ID NO: 1499, for instance, where the M97 protein is comprised within a fusion protein. In other embodiments, the nucleotide sequence encoding the M97 protein comprises SEQ ID NO: 1499, including or lacking the start codon or stop codon.

In some embodiments, the enhancer is a KSHV ORF36 protein. In some embodiments, the KSHV ORF36 protein comprises an amino acid sequence that has at least 90%, 91%, 92%, 93%, 94%, 95%, 96%, 97%, 98%, 99%, or 100% identity to SEQ ID NO: 1576. In some embodiments, the KSHV ORF36 protein is encoded by a nucleotide sequence that has at least 90%, 91%, 92%, 93%, 94%, 95%, 96%, 97%, 98%, 99%, or 100% identity to SEQ ID NO: 1575. In some embodiments, the amino acid sequence of the KSHV ORF36 protein lacks an N-terminal methionine, for instance, where the KSHV ORF36 protein is comprised within a fusion protein. In some embodiments, the amino acid sequence of the KSHV ORF36 protein lacks the N-terminal methionine of SEQ ID NO: 1576, for instance, where the KSHV ORF36 protein is comprised within a fusion protein. In other embodiments, the amino acid sequence of the KSHV ORF36 protein comprises SEQ ID NO: 1576, including or lacking the N-terminal methionine. In some embodiments, the nucleotide sequence encoding the KSHV ORF36 protein lacks the start codon or stop codon. In some embodiments, the nucleotide sequence encoding the KSHV ORF36 protein lacks the start codon or stop codon of SEQ ID NO: 1575, for instance, wherein the KSHV ORF36 protein is comprised within a fusion protein. In other embodiments, the nucleotide sequence encoding the KSHV ORF36 protein comprises SEQ ID NO: 1575, including or lacking the start codon or stop codon.

In some embodiments, the enhancer is a DNA repair protein. In some embodiments, the DNA repair protein is a single-stranded endonuclease that removes flaps with an available 5′ end 3′ of a nick. In some embodiments, the DNA repair protein is flap structure-specific endonuclease 1 (FEN1) or a polynucleotide encoding FEN1. In one embodiment, FEN1 may be provided by contacting a cell with a nucleic acid (e.g., an mRNA) encoding a FEN1 polypeptide. In some embodiments, the FEN1 comprises an amino acid sequence that has at least 90%, 91%, 92%, 93%, 94%, 95%, 96%, 97%, 98%, 99%, or 100% identity to any one of SEQ ID NOs: 1467, 1475, 1485, 1490, 1495, and 1510-1516. In some embodiments, the FEN1 is encoded by a nucleotide sequence that has at least 90%, 91%, 92%, 93%, 94%, 95%, 96%, 97%, 98%, 99%, or 100% identity to SEQ ID NO: 1466, 1474, 1479, 1484, 1489, 1494, 1509, and 1524-1529. In some embodiments, the amino acid sequence of the FEN1 protein lacks an N-terminal methionine, for instance, where the FEN1 protein is comprised within a fusion protein. In some embodiments, the amino acid sequence of the FEN1 protein lacks the N-terminal methionine of any one of SEQ ID NOs: 1467, 1475, 1485, 1490, 1495, and 1510-1516, for instance, where the FEN1 protein is comprised within a fusion protein. In other embodiments, the amino acid sequence of the FEN1 protein comprises any one of SEQ ID NOs: 1467, 1475, 1485, 1490, 1495, and 1510-1516, including or lacking the N-terminal methionine. In some embodiments, the nucleotide sequence of the FEN1 protein lacks a start codon or stop codon. In some embodiments, the nucleotide sequence of the FEN1 protein lacks the start codon or stop codon of any one of SEQ ID NOs: 1466, 1474, 1479, 1484, 1489, 1494, 1509, and 1524-1529, for instance, wherein the FEN1 protein is comprised within a fusion protein. In other embodiments, the nucleotide sequence encoding the FEN1 protein comprises any one of SEQ ID NOs: 1466, 1474, 1479, 1484, 1489, 1494, 1509, and 1524-1529, including or lacking the start codon or stop codon.

In some embodiments, the enhancer comprises a dNTP synthetic enzyme. In some embodiments, the enhancer comprises a kinase. In some embodiments, the kinase comprises a nucleoside kinase, deoxynucleoside kinase, deoxynucleoside monophosphate kinase, or deoxynucleotide diphosphate.

In some embodiments, the enhancer comprises Vpx, BGLF4, M97, KSHV ORF36, or FEN1. In some embodiments, the enhancer comprises Vpx and FEN1. In some embodiments, the enhancer comprises M97 and FEN1. In some embodiments, the FEN1 comprises a heterodimerization domain, e.g., a self-assembling coiled coil heterodimerization domain, for binding to a DNA-dependent DNA polymerase having the sequence of the other portion of the heterodimerization domain.

In some embodiments, the enhancer comprises a heterologous NLS. In some embodiments, the enhancer comprises a C-terminal heterologous NLS. In some embodiments, the enhancer comprises an N-terminal heterologous NLS. In some embodiments, the enhancer comprises a C-terminal heterologous NLS and a C-terminal heterologous NLS. In some embodiments, the enhancer does not comprise a heterologous NLS.

In some embodiments, the enhancer confers an increase in gene editing. In some embodiments, the enhancer confers a two-fold increase in gene editing. In some embodiments, the enhancer confers a three-fold increase in gene editing. In some embodiments, the enhancer confers a four-fold increase in gene editing. In some embodiments, the enhancer confers a five-fold increase in gene editing. In some embodiments, the enhancer confers a ten-fold increase in gene editing.

In some embodiments, a DNA binding accessory factor is in a composition, system, or kit with a DNA-dependent DNA polymerase when the polymerase is present in combination with a SpyCas9 nickase. In some embodiments, the composition comprises an Sso7 DNA binding accessory factor in combination with a DNA-dependent DNA polymerase when the polymerase is present in combination with a SpyCas9 nickase.

### Compositions and Kits Comprising Agents to Increase Template-Based Editing

In some embodiments, compositions, pharmaceutical formulations, kits, methods, or uses are provided comprising (a) a SpyCas9 nickase or a nucleic acid encoding the same, (b) a DNA-dependent DNA polymerase or a nucleic acid encoding the same, (c) a template guide RNA for template-based genome editing, and optionally (d) an agent for increasing template-based editing. In certain embodiments, the agent for increasing template-based editing is an agent to increase dNTP concentration in the cell. In certain embodiments, the agent for increasing template-based editing is a DNA repair protein. In certain embodiments, the agent to increase template-based editing is a combination of an agent to increase dNTP concentration in the cell and a DNA repair protein. In certain embodiments, the agent for increasing template-based editing is a nicking guide. In certain embodiments, the agent to increase template-based editing is a non-specific DNA binding protein.

In certain embodiments, the agent is for increasing the dNTP concentration in a cell. For example, inhibiting SAMHD1 may increase the dNTP concentration. Some embodiments include compositions or pharmaceutical formulations for increased genome editing efficiency comprising inhibiting SAMHD1 in a cell. In certain embodiments, the inhibitor of SAMHD1 is an siRNA, shRNA, or microRNA targeted to SAMHD1.

In some embodiments, a composition, kit, use, or method of the present disclosure may comprise a protein or a nucleic acid encoding the protein for increasing editing efficiency of one or more constructs of the present disclosure (e.g., a SpyCas9 nickase/DNA-dependent DNA polymerase fusion construct). In some embodiments, a protein or a nucleic acid encoding the protein for increasing editing efficiency of a SpyCas9 nickase/DNA-dependent DNA polymerase fusion construct may comprise a protein that inhibits the dNTP cleavage activity of SAMHD1 or a nucleic acid encoding a protein that inhibits the dNTP cleavage activity of SAMHD1 e.g., Vpx, BGLF4, M97, or KSHV ORF36.

Some embodiments include compositions, systems, pharmaceutical formulations, kits, methods or uses include agents that increase the dNTP concentration in the cell, relative to a baseline dNTP concentration.

In various aspects, the present disclosure provides compositions, systems, pharmaceutical formulations, kits, uses, and methods for increased gene editing efficiency in a cell, e.g., a quiescent cell or typically non-dividing cell expected to have a low deoxynucleoside triphosphate (dNTP) concentration and comprising a DNA polymerase, wherein the composition or pharmaceutical formulation increases the dNTP concentration in the cell relative to a baseline dNTP concentration. In various aspects, increasing the dNTP concentration in the cell comprises inhibiting a deoxynucleotide triphosphate triphosphohydrolase in the cell. In various aspects, the deoxynucleotide triphosphate triphosphohydrolase comprises SAM domain and HD domain-containing protein 1 (SAMHD1). In various aspects, inhibiting SAMHD1 comprises contacting the SAMHD1 with a Vpx protein, a BGLF4 protein, an M97 protein, or a KSHV ORF36 protein, or expressing the Vpx protein, the BGLF4 protein, the M97 protein, or the KSHV ORF36 protein in the cell. In various aspects, the low dNTP concentration comprises a dNTP concentration found in a nondividing cell or in an activated peripheral blood mononuclear cell.

Disclosed herein are compositions, systems, and pharmaceutical formulations for increased genome editing efficiency. The compositions and pharmaceutical formulations may inhibit SAMHD1 in a cell. The cell may express (a) a SpyCas9 nickase or a nucleic acid encoding the same, (b) a DNA-dependent DNA polymerase or a nucleic acid encoding the same, and (c) a template guide RNA for template-based genome editing. An example of inhibiting SAMHD1 may include treating the cell with a SAMHD1 inhibitor such as a protein. An example of inhibiting SAMHD1 may include expressing a Vpx protein, a BGLF4 protein, an M97 protein, or a KSHV ORF36 protein in the cell.

A protein for increasing editing efficiency may be a Vpx protein (e.g., any one of SEQ ID NOs: 1464, 1469, and 1503-1505 or an amino acid sequence having at least 90%, 91%, 92%, 93%, 94%, 95%, 96%, 97%, 98%, 99%, or 100% identity to SEQ ID NOs: 1464, 1469, and 1503-1505). Vpx is in some instances a lentiviral protein. Vpx is in some instances a immunodeficiency virus (SIV) protein which may be used for increasing editing efficiency (e.g. by inhibiting SAMHD1). A Vpx peptide may be an HIV Vpx (e.g., SEQ ID NO: 1504) or an amino acid sequence having at least 90%, 91%, 92%, 93%, 94%, 95%, 96%, 97%, 98%, 99%, or 100% identity to SEQ ID NO: 1504. In some embodiments, a Vpx protein may be expressed as its own coding sequence. In some embodiments, a Vpx protein may be expressed in the same coding sequence as the SpyCas9 nickase or DNA-dependent DNA polymerase. For example, a Vpx protein may be expressed in the same coding sequence as the SpyCas9 nickase or DNA-dependent DNA polymerase, separated by a P2A or T2A self-cleaving peptide. In some embodiments, a Vpx protein may be expressed in the same coding sequence as the SpyCas9 protein.

A protein for increasing editing efficiency may be a BGLF4 protein (e.g., SEQ ID NO: 1517 or 1518 or an amino acid sequence having at least 90%, 91%, 92%, 93%, 94%, 95%, 96%, 97%, 98%, 99%, or 100% identity to SEQ ID NO: 1517 or 1518).

A protein for increasing editing efficiency may be an M97 protein (e.g., SEQ ID NO: 1500 or an amino acid sequence having at least 90%, 91%, 92%, 93%, 94%, 95%, 96%, 97%, 98%, 99%, or 100% identity to SEQ ID NO: 1500); or a protein encoded by a nucleic acid sequence of SEQ ID NO: 1499 or a nucleic sequence having at least 90%, 91%, 92%, 93%, 94%, 95%, 96%, 97%, 98%, 99%, or 100% identity to SEQ ID NO: 1499.

A protein for increasing editing efficiency may be a KSHV ORF36 protein (e.g., SEQ ID NO: 1576 or an amino acid sequence having at least 90%, 91%, 92%, 93%, 94%, 95%, 96%, 97%, 98%, 99%, or 100% identity to SEQ ID NO: 1576).

Inhibiting SAMHD1 may comprise expressing a Vpx protein, a BGLF4 protein, an M97, or a KSHV ORF36 protein in the cell. For example, in one embodiment, inhibiting SAMHD1 may comprise providing a cell with a nucleic acid (e.g., an mRNA) encoding a Vpx polypeptide.

Disclosed herein are compositions, systems, and pharmaceutical formulations for increased genome editing efficiency comprising a Vpx protein, a BGLF4 protein, an M97 protein, or a KSHV ORF36 protein, wherein the Vpx protein, BGLF4 protein, M97 protein, or KSHV ORF36 is expressed in a cell. The cell may express a composition described herein. The cell may express a guide nucleic acid described herein.

Some embodiments include compositions, systems, and pharmaceutical formulations for increased dNTP concentration in a cell, or of improved gene editing, wherein the composition or pharmaceutical formulation inhibits a deoxynucleotide triphosphate triphosphohydrolase (dNTPase) such as SAMHD1. Some embodiments relate to a composition for inhibiting SAMHD1. A Vpx protein, BGLF4 protein, M97 protein, or KSHV ORF36 protein may be used to inhibit SAMHD1.

In certain embodiments, the compositions, pharmaceutical formulations, kits, methods, or uses include more than one enhancer of template-based editing. In certain embodiments, the enhancers of template-based editing comprise an agent to increase dNTP in a cell and a DNA repair protein, or nucleic acid encoding the same. In certain embodiments the enhancers of template-based editing comprise Vpx (e.g., Vpx-1) and FEN1. In certain embodiments, the enhancers of template-based editing comprise M97 and FEN1. In some embodiments, the FEN1 comprises a heterodimerization domain, e.g., a self-assembling coiled coil heterodimerization domain, for binding to a DNA-dependent DNA polymerase having the sequence of the other portion of the heterodimerization domain.

In certain embodiments, Vpx (e.g., Vpx-1) and FEN1 are encoded on separate polynucleotides. In certain embodiments, Vpx and FEN1 are encoded on the same polynucleotide. In certain embodiments, Vpx and FEN1 are encoded on the same polynucleotide, e.g., on the same polynucleotide as a fusion protein. In some embodiments, wherein Vpx and FEN1 are encoded on the same polynucleotide as a fusion protein, the fusion protein comprises a peptide cleavage site between Vpx and FEN1. In some embodiments, the fusion protein comprises an amino acid sequence that has at least 90%, 91%, 92%, 93%, 94%, 95%, 96%, 97%, 98%, 99%, or 100% identity to any one of SEQ ID NOs: 1578, 1580, 1582, and 1584. In some embodiments, the fusion protein is encoded by a nucleic acid sequence that has at least 90%, 91%, 92%, 93%, 94%, 95%, 96%, 97%, 98%, 99%, or 100% identity to any one of SEQ ID NOs: 1577, 1579, 1581, and 1583, 3523, 3524, 3526, and 3527.

Disclosed herein are compositions, systems, and pharmaceutical formulations for increased genome editing efficiency comprising a nicking guide as described herein.

Disclosed herein are compositions, systems, and pharmaceutical formulations for increased genome editing efficiency comprising a non-specific DNA binding protein, e.g. a low molecular weight non-specific DNA binding protein. In certain embodiments, the non-specific DNA binding protein is Sso7, a small protein (7 kD) capable of covalently binding to dsDNA without any preference for specific sequences. The binding of the Sso7d domain to a DNA polymerase, is able to smoothly slide along the template. In certain embodiments, the Sso7 polypeptide is covalently attached to the polymerase. In some embodiments, the Sso7 polypeptide comprises an amino acid sequence that has at least 90%, 91%, 92%, 93%, 94%, 95%, 96%, 97%, 98%, 99%, or 100% identity to SEQ ID NO: 1547.

### Compositions and Kits Comprising a DNA Repair Protein

In some embodiments, compositions, systems, pharmaceutical formulations, kits, methods, or uses are provided comprising (a) a SpyCas9 nickase or a nucleic acid encoding the same, (b) a DNA-dependent DNA polymerase or a nucleic acid encoding the same, (c) a template guide RNA for template-based genome editing, and (d) a DNA repair protein, or nucleic acid encoding the same.

In some embodiments, the DNA repair protein is an agent for removing a displaced flap of endogenous DNA may be a flap endonuclease. As used herein, the term “flap endonuclease” refers to an enzyme that catalyzes the removal from the 5′ end of single strand DNA flaps generated at the 3′ ends of nicks. These are naturally occurring enzymes that process the removal from the 5′ end of flaps generated at the 3′ ends of nicks formed during cellular processes, including DNA replication. The template-based genome editing methods described herein may utilize endogenously supplied flap endonucleases or those provided in trans to remove from the 3′ end the flap generated at the 3′ end of a nick in the endogenous

DNA formed at the target site during template-based genome editing. Flap endonucleases are known in the art and can be found described in Patel et al., “Flap endonucleases pass 5′-flaps through a flexible arch using a disorder-thread-order mechanism to confer specificity for free 5′-ends,” Nucleic Acids Research, 2012, 40 (10): 4507-4519, Tsutakawa et al., “Human flap endonuclease structures, DNA double-base flipping, and a unified understanding of the FEN1 superfamily,” Cell, 2011, 145(2): 198-211, and Balakrishnan et al., “Flap Endonuclease 1,” Annu Rev Biochem, 2013, Vol 82: 119-138 (each of which are incorporated herein by reference). An exemplary flap endonuclease is FEN1.

In some embodiments, the FEN1 is an animal FEN1. In some embodiments, the FEN1 is a vertebrate FEN1. In some embodiments, the FEN1 is a mammalian FEN1. In some embodiments, the FEN1 is a human FEN1. In some embodiments, wherein the FEN1 is a human FEN1, the FEN1 comprises an amino acid sequence that has at least 90%, 91%, 92%, 93%, 94%, 95%, 96%, 97%, 98%, 99%, or 100% identity to any one of SEQ ID NOs: 1475 and 1510-1512. In some embodiments, wherein the FEN1 is a human FEN1, the FEN1 comprises a D181A mutation relative to SEQ ID NO: 1510. In some embodiments, the FEN1 is a cynomolgus FEN1. In some embodiments, wherein the FEN1 is a cynomolgus FEN1, the FEN1 comprises an amino acid sequence that has at least 90%, 91%, 92%, 93%, 94%, 95%, 96%, 97%, 98%, 99%, or 100% identity to SEQ ID NO: 1514. In some embodiments, the FEN1 is a rodent FEN1. In some embodiments, the FEN1 is a mouse FEN1. In some embodiments, wherein the FEN1 is a mouse FEN1, the mouse FEN1 comprises an amino acid sequence that has at least 90%, 91%, 92%, 93%, 94%, 95%, 96%, 97%, 98%, 99%, or 100% identity to SEQ ID NO: 1513. In some embodiments, the FEN1 is a turtle FEN1. In some embodiments, the FEN1 is a Chrysemys picta bellii FEN1. In some embodiments, wherein the FEN1 is a C. picta bellii FEN1, the FEN1 comprises an amino acid sequence that has at least 90%, 91%, 92%, 93%, 94%, 95%, 96%, 97%, 98%, 99%, or 100% identity to SEQ ID NOs: 1516 and 1485.

In some embodiments, the flap endonuclease is a fungal flap endonuclease. In some embodiments, the flap endonuclease is a yeast flap endonuclease. In some embodiments, the flap endonuclease is a Schizosaccharomyces pombe flap endonuclease. In some embodiments, wherein the flap endonuclease is an S. pombe flap endonuclease, the flap endonuclease is RAD2. In some embodiments, wherein the flap endonuclease is RAD2, the flap endonuclease comprises an amino acid sequence that has at least 90%, 91%, 92%, 93%, 94%, 95%, 96%, 97%, 98%, 99%, or 100% identity to SEQ ID NO: 1515.

In some embodiments, the FEN1 comprises a heterologous NLS. In some embodiments, the FEN1 does not comprise a heterologous NLS. In some embodiments, the FEN1 is recruited to the nick site of the tgRNA. In some embodiments, the FEN1 comprises a heterodimerization domain, e.g., a self-assembling coiled coil heterodimerization domain, for binding to a DNA-dependent DNA polymerase having the sequence of the other portion of the heterodimerization domain.

In some embodiments, the DNA repair protein is MHL1 protein is mutL homolog 1 (MLH1). In some embodiments, MLH1 comprises an amino acid sequence that has at least 90%, 91%, 92%, 93%, 94%, 95%, 96%, 97%, 98%, 99%, or 100% identity to SEQ ID NO: 1572 or 1573. The MLH1 protein can heterodimerize with mismatch repair endonuclease PMS2 to form MutL alpha, part of the DNA mismatch repair system. When MutL alpha is bound by MutS beta and some accessory proteins, the PMS2 subunit of MutL alpha introduces a single-strand break near DNA mismatches, providing an entry point for exonuclease degradation.

In some embodiments, the DNA repair protein comprises a heterodimerization domain or further comprises an additional heterodimerization domain. In some embodiments, the heterodimerization domain comprises an EV, EI, KV, or KI domain. In some embodiments, the heterodimerization domain comprises an EI or KI domain. In some embodiments, the heterodimerization domain comprises an EI domain. In some embodiments, the heterodimerization domain comprises a KI domain. In some embodiments, the heterodimerization domain comprises SEQ ID NO: 1589, SEQ ID NO: 1593, or SEQ ID NO: 1595. In such embodiments, the DNA-dependent DNA polymerase comprises the corresponding heterodimerization domain to the heterodimerization domain present on the DNA repair protein to allow dimerization of the DNA repair protein and the DNA-dependent DNA polymerase.

### Exemplary Nucleic Acid Compositions

In some embodiments, provided herein is one or more nucleic acid (e.g., mRNA) encoding a polypeptide disclosed herein. In some embodiments, the DNA-dependent DNA polymerase, nCas9, and enhancers are provided as one or more nucleic acids. In some embodiments, the present disclosure provides for one or more nucleic acid (e.g., mRNA) encoding a polypeptide (a) a SpyCas9 nickase (nCas9) and (b) a DNA-dependent DNA polymerase.

In some embodiments, the present disclosure provides for one or more nucleic acid (e.g., mRNA) encoding a polypeptide comprising a first split intein and an nCas9. In some embodiments, the present disclosure provides for one or more nucleic acid (e.g., mRNA) encoding a polypeptide comprising a second split intein and a DNA-dependent DNA polymerase. In some embodiments, the first split intein is an N-intein and the second split intein is a C-intein. In some embodiments, the first split intein construct is a C-intein and the second split intein construct is an N-intein. In some embodiments, the present disclosure provides for a polynucleotide (e.g., mRNA) comprising an ORF encoding a polypeptide comprising the SpyCas9 nickase or portion thereof and an N-intein. In some embodiments, the present disclosure provides for a polynucleotide (e.g., mRNA) comprising an ORF encoding a polypeptide comprising the DNA-dependent DNA polymerase, or portion thereof, and a C-intein. In some embodiments, the present disclosure provides for a nucleic acid (e.g., mRNA) comprising an ORF encoding a polypeptide comprising the SpyCas9 nickase or portion thereof, and a N-intein; and (2) a nucleic acid (e.g., mRNA) comprising an ORF encoding a polypeptide comprising the DNA-dependent DNA polymerase or portion thereof, and a C-intein.

In some embodiments, the polypeptide comprises at least one enhancer of template-based genome editing disclosed herein. Non-limiting examples of enhancers of template-based genome editing include Vpx, BGLF4, M97, KSHV ORF36, and FEN1. In some embodiments, the enhancer is Vpx. In some embodiments, the enhancer is FEN1. In some embodiments, the enhancers are Vpx and FEN1. In some embodiments, the polypeptide further comprises a heterodimerization domain, optionally a self-assembling coiled coil heterodimerization domain, e.g., a KI domain or an EI domain, in each of the DNA-dependent DNA polymerase and enhancer to promote heterodimerization of the DNA-dependent DNA polymerase and the enhancer. In some embodiments, the polypeptide further comprises at least one heterologous NLS.

In some embodiments, the one or more nucleic acid (e.g., mRNA) disclosed herein comprises one or more nucleotide sequence encoding a heterologous NLS independently selected from a nucleoplasmin NLS, C-Myc NLS, and SV40 NLS. In some embodiments, the nucleic acid (e.g., mRNA) disclosed herein comprises one or more nucleotide sequence encoding an NLS, the one or more nucleotide sequence independently selected from SEQ ID NOs: 1726, 1727, 1729, 1805, 1806, 1807, 1808, 1810, 1831.

In some embodiments, the nucleic acid (e.g., mRNA) disclosed herein comprises a Cap; a 5′ UTR; a Kozak sequence; a 3′ UTR, a poly-A tail; or combinations thereof. Non-limiting examples for each element for the mRNA encoding a system or polypeptide comprising an nCas9 and a DNA-dependent DNA polymerase are provided in Table 4A.

In some embodiments, the nucleic acid (e.g., mRNA) disclosed herein comprises a cap selected from Cap0 and Cap1. In some embodiments, the nucleic acid (e.g., mRNA) disclosed herein comprises a 5′ UTR selected from SEQ ID NOs: 1720-1723. In some embodiments, the nucleic acid (e.g., mRNA) disclosed herein comprises a Kozak sequence selected from SEQ ID NOs: 1724 and 1725. In some embodiments, the nucleic acid (e.g., mRNA) disclosed herein comprises a 3′ UTR selected from SEQ ID NOs: 1684 and 1740. In some embodiments, the nucleic acid (e.g., mRNA) disclosed herein comprises a poly-adenylated (poly-A) tail sequence selected from SEQ ID NOs: 1741 and 1742.

In some embodiments, the nucleic acid (e.g., mRNA) disclosed herein comprises:
i.a Cap selected from Cap0 or Cap1;
ii.a 5′ UTR selected from SEQ ID NOs: 1720-1723;
iii.a Kozak sequence selected from SEQ ID NOs: 1724 and 1725;
iv.a 3′ UTR selected from SEQ ID NOs: 1684 and 1740;
v.a poly-A tail selected from SEQ ID NOs: 1741 and 1742; or
vi.a combination of two, three, four, or five of (i)-(v).

In some embodiments, the nucleic acid (e.g., mRNA) encoding the nCas9 and the DNA-dependent DNA polymerase disclosed herein comprises, from 5′ to 3′ (1) a 5′ Cap; (2) a 5′ UTR; (3) a Kozak sequence; (4) a coding sequence encoding (i) an nCas9; (ii) a DNA polymerase; (iii) at least one heterologous NLS; (iv) a first linker, optionally comprising a heterologous NLS; (v) an optional second linker, optionally comprising a heterologous NLS or a heterodimerization domain; (5) a 3′UTR; and (6) a poly-A tail. Non-limiting examples for the mRNAs disclosed herein are provided in Table 4B. In some embodiments, the nucleic acid (e.g., mRNA) disclosed herein comprises a nucleotide sequence having at least 85%, 86%, 87%, 88%, 89%, 90%, 91%, 92%, 93%, 94%, 95%, 96%, 97%, 98%, 99%, or 100% identity to any one of SEQ ID NOs: 2586-2870.

Non-limiting examples for the mRNAs encoding a polypeptide comprising a split intein disclosed herein are provided in Table 4C. In some embodiments, the one or more nucleic acid (e.g., mRNA) disclosed herein comprises a nucleotide sequence having at least 85%, 86%, 87%, 88%, 89%, 90%, 91%, 92%, 93%, 94%, 95%, 96%, 97%, 98%, 99%, or 100% identity to any one of SEQ ID NOs: 3001-3022. In some embodiments, the present disclosure provides for a polynucleotide (e.g., mRNA) comprising an ORF encoding a polypeptide comprising the SpyCas9 nickase or portion thereof and an N-intein, comprising a nucleotide sequence having at least 85%, 86%, 87%, 88%, 89%, 90%, 91%, 92%, 93%, 94%, 95%, 96%, 97%, 98%, 99%, or 100% identity to any one of SEQ ID NOs: 3006, 3007, 3008, 3010, 3012, 3017, 3018. In some embodiments, the present disclosure provides for a polynucleotide (e.g., mRNA) comprising an ORF encoding a polypeptide comprising the DNA-dependent DNA polymerase, or portion thereof, and a C-intein comprising a nucleotide sequence having at least 85%, 86%, 87%, 88%, 89%, 90%, 91%, 92%, 93%, 94%, 95%, 96%, 97%, 98%, 99%, or 100% identity to any one of SEQ ID NOs: 3001-3005, 3009, 3011, 3013-3016, 3019-3022. In some embodiments, the present disclosure provides for a first polynucleotide (e.g., mRNA) comprising an ORF encoding a polypeptide comprising the SpyCas9 nickase or portion thereof and an N-intein, comprising a nucleotide sequence having at least 85%, 86%, 87%, 88%, 89%, 90%, 91%, 92%, 93%, 94%, 95%, 96%, 97%, 98%, 99%, or 100% identity to any one of SEQ ID NOs: 3006, 3007, 3008, 3010, 3012, 3017, 3018 and a second polynucleotide (e.g., mRNA) comprising an ORF encoding a polypeptide comprising the DNA-dependent DNA polymerase, or portion thereof, and a C-intein comprising a nucleotide sequence having at least 85%, 86%, 87%, 88%, 89%, 90%, 91%, 92%, 93%, 94%, 95%, 96%, 97%, 98%, 99%, or 100% identity to any one of SEQ ID NOs: 3001-3005, 3009, 3011, 3013-3016, 3019-3022.

In some embodiments, the present disclosure provides for a first polynucleotide (e.g., mRNA) comprising an ORF encoding a polypeptide comprising the SpyCas9 nickase or portion thereof and an CfaN split intein, comprising a nucleotide sequence having at least 85%, 86%, 87%, 88%, 89%, 90%, 91%, 92%, 93%, 94%, 95%, 96%, 97%, 98%, 99%, or 100% identity to any one of SEQ ID NOs: 3006, 3007, 3008, 3010, 3017, 3018 and a second polynucleotide (e.g., mRNA) comprising an ORF encoding a polypeptide comprising the DNA-dependent DNA polymerase, or portion thereof, and a CfaC split intein comprising a nucleotide sequence having at least 85%, 86%, 87%, 88%, 89%, 90%, 91%, 92%, 93%, 94%, 95%, 96%, 97%, 98%, 99%, or 100% identity to any one of SEQ ID NOs: 3001-3005, 3009, 3011, 3014-3016, 3019-3022.

In some embodiments, the present disclosure provides for a first polynucleotide (e.g., mRNA) comprising an ORF encoding a polypeptide comprising the SpyCas9 nickase or portion thereof and an NpuN split intein, comprising a nucleotide sequence having at least 85%, 86%, 87%, 88%, 89%, 90%, 91%, 92%, 93%, 94%, 95%, 96%, 97%, 98%, 99%, or 100% identity to SEQ ID NO: 3012 and a second polynucleotide (e.g., mRNA) comprising an ORF encoding a polypeptide comprising the DNA-dependent DNA polymerase, or portion thereof, and a NpuC split comprising a nucleotide sequence having at least 85%, 86%, 87%, 88%, 89%, 90%, 91%, 92%, 93%, 94%, 95%, 96%, 97%, 98%, 99%, or 100% identity to SEQ ID NO: 3013.

In some embodiments, the nucleic acid (e.g., mRNA) encoding an enhancer of template-based genome editing disclosed herein comprises (1) a 5′ Cap; (2) a 5′ UTR; (3) a Kozak sequence; (4) a coding sequence encoding at least one enhancer and optionally a linker, optionally comprising a heterologous NLS, a heterodimerization domain, or P2A or T2A self-cleaving peptide; (5) a 3′ UTR; and (6) a poly-A tail. Non-limiting examples for the mRNAs disclosed herein are provided in Table 4D. In some embodiments, the one or more nucleic acid (e.g., mRNA) encoding an enhancer of template-based genome editing disclosed herein comprises a nucleotide sequence having at least 85%, 86%, 87%, 88%, 89%, 90%, 91%, 92%, 93%, 94%, 95%, 96%, 97%, 98%, 99%, or 100% identity to any one of SEQ ID NOs: 3516-3530.

TABLE 4A
Exemplary elements for nucleic acid (or mRNA) constructs provided herein
(Note that for some of the elements, alternative DNA sequences encoding the same amino
acid sequence may be provided.)

| mRNA | Exemplary | | Amino Acid Sequence |
|---|---|---|---|
| region | element | DNA Sequence (SEQ ID NO) | (SEQ ID NO) |
| Cap | Cap0 | | |
| | Cap1 | | |
| 5′ UTR | 5′_XBG | AAGCTCAGAATAAACGCTCAACTTTG | N/A |
| | | GCCGGATCT (SEQ ID NO: 1720) | |
| | 4GApt1 | ACTCACTATTTGTTTTCGCGCCCAGT | N/A |
| | | TGCAAAAAGTGTCG (SEQ ID | |
| | | NO: 1721) | |
| | 4EApt1 | TGTTCAACCAGAGTGAAACCACTAAC | N/A |
| | | GGGTCAGAGCCCC (SEQ ID | |
| | | NO: 1722) | |
| | PABPv3 | AAAAAAAAAAAACCAAAAAAAAAAAA | N/A |
| | | CAAAAAAAAAAAATAATTGACTAA | |
| | | (SEQ ID NO: 1723) | |
| Kozak | Kozak V1 | GCCACCATGA (SEQ ID | N/A |
| | | NO: 1724) | |
| | Kozak V2 | GCCACCATGG (SEQ ID | N/A |
| | | NO: 1725) | |
| NLS | Nucleoplasmin | AAGCGGCCCGCCGCCACCAAGAAGGC | KRPAATKKAGQAKKKK (SEQ ID |
| | | CGGCCAGGCCAAGAAGAAGAAG | NO: 1706) |
| | | (SEQ ID NO: 1726) | |
| | Nucleoplasmin | AAGCGGCCGGCTGCGACCAAGAAGGC | KRPAATKKAGQAKKKK (SEQ ID |
| | | TGGTCAGGCTAAGAAGAAGAAG | NO: 1706) |
| | | (SEQ ID NO: 1805) | |
| | cMyc | CCTGCCGCTAAGAGAGTGAAGCTGGA | PAAKRVKLD (SEQ ID |
| | | C (SEQ ID NO: 1727) | NO: 1728) |
| | cMyc | CCCGCTGCGAAGCGGGTAAAGCTTGA | PAAKRVKLD (SEQ ID |
| | | T (SEQ ID NO: 1806) | NO: 1728) |
| | SV40 | CCCAAGAAGAAGCGGAAGGTG (SEQ | PKKKRKV (SEQ ID NO: 1671) |
| | | ID NO: 1807) | |
| | SV40 | CCGAAAAAGAAGCGGAAGGTT (SEQ | PKKKRKV (SEQ ID NO: 1671) |
| | | ID NO: 1729) | |
| | bpSV40 | AAGCGGACCGCCGACGGCTCCGAGTT | KRTADGSEFESPKKKRKV (SEQ |
| | | CGAGTCCCCGAAAAAGAAGCGGAAGG | ID NO: 1809) |
| | | TG (SEQ ID NO: 1808) | |
| | bpSV40 | AAGAGGACTGCGGATGGTTCTGAATT | KRTADGSEFESPKKKRKV (SEQ |
| | | CGAATCACCTAAGAAGAAGCGGAAGG | ID NO: 1809) |
| | | TC (SEQ ID NO: 1810) | |
| | bpSV40 | AAGCGGACTGCGGACGGGTCCGAGTT | KRTADGSEFESPKKKRKV (SEQ |
| | | CGAGTCTCCGAAGAAGAAGCGCAAGG | ID NO: 1809) |
| | | TC (SEQ ID NO: 1831) | |
| linker | G4S | GGGGGTGGGGGAAGT (SEQ ID | GGGGS (SEQ ID NO: 1605) |
| | | NO: 1736) | |
| | SGGS | TCCGGCGGCTCC (SEQ ID | SGGS (SEQ ID NO: 1738) |
| | | NO: 1737) | |
| | Linker14 (L14) | TCCGGCGGCTCCAAGCGGACCGCCGA | SGGSKRTADGSEFE (SEQ ID |
| | | CGGCTCCGAGTTCGAG (SEQ ID | NO: 1596) |
| | | NO: 1731) | |
| | Linker16 (L16) | GGCGGAGGCTCAGGCAAGCGGACCGC | GGGSGKRTADGSGGGS (SEQ ID |
| | | TGACGGCTCCGGGGGGGGCTCT | NO: 1597) |
| | | (SEQ ID NO: 1730) | |
| | L16 | GGCGGGGGCTCCGGCAAGCGCACCGC | GGGSGKRTADGSGGGS (SEQ ID |
| | | TGACGGCTCCGGGGGGGGCTCT | NO: 1597) |
| | | (SEQ ID NO: 1811) | |
| | L16 | GGCGGCGGCTCCGGCAAGCGGACCGC | GGGSGKRTADGSGGGS (SEQ ID |
| | | CGACGGCTCCGGCGGCGGCTCC | NO: 1597) |
| | | (SEQ ID NO: 1812) | |
| | Linker41 (L41) | GGCGCCCCCGAGTCCGCCACCGAGTC | GAPESATESGGTSTESEGSAGTSTE |
| | | CGGCGGCACCTCCACCGAGTCCGAGG | SEGSAGSAGSTSGSSS (SEQ ID |
| | | GCTCCGCCGGCACCTCCACCGAGTCC | NO: 1735) |
| | | GAGGGCTCCGCCGGCTCCGCCGGCTC | |
| | | CACCTCCGGCTCCTCCTCC (SEQ | |
| | | ID NO: 1734) | |
| | Linker42 (L42) | GGCGCCCCCGAGTCCGCCACCGAGTC | GAPESATESGGTSTESEGSAGPKKK |
| | | CGGCGGCACCTCCACCGAGTCCGAGG | RKVTSTESEGSAGSAGSTSGSSSG |
| | | GCTCCGCCGGCCCCAAGAAGAAGCGG | (SEQ ID NO: 1600) |
| | | AAGGTGACCTCCACCGAGTCCGAGGG | |
| | | CTCCGCCGGCTCCGCCGGCTCCACCT | |
| | | CCGGCTCCTCCTCCGGC (SEQ ID | |
| | | NO: 1732) | |
| | L42(SV40)- | GGCGCCCCCGAGTCTGCGACAGAGTC | GAPESATESGGTSTESEGSAGPKKK |
| | linker42 - | GGGCGGGACTTCCACCGAGAGTGAAG | RKVTSTESEGSAGSAGSTSGSSSG |
| | comprising a | GATCGGCCGGCCCCAAGAAGAAGCGC | (SEQ ID NO: 1600) |
| | SV40 NLS | AAAGTCACGTCGACGGAGTCGGAGGG | |
| | | GTCTGCGGGGTCCGCGGGGAGCACGT | |
| | | CCGGCTCCTCCTCGGGC (SEQ ID | |
| | | NO: 1813) | |
| | Linker42 (L42) | GGCGCCCCCGAGTCCGCCACCGAGTC | GAPESATESGGTSTESEGSAGTSTE |
| | | CGGCGGCACCTCCACCGAGTCCGAGG | SEGSAGSAGSTSGSSSS (SEQ ID |
| | | GCTCCGCCGGCACCTCCACCGAGTCC | NO: 1815) |
| | | GAGGGCTCCGCCGGCTCCGCCGGCTC | |
| | | CACCTCCGGCTCCTCCTCCTCC | |
| | | (SEQ ID NO: 1814) | |
| | GH5 | GGTACCAAGGACAGCACCAAAGATAT | GTKDSTKDIPETPSKD (SEQ ID |
| | | CCCGGAAACCCCGAGCAAGGAC | NO: 1661) |
| | | (SEQ ID NO: 1733) | |
| | GH5 | GGCACCAAGGACTCCACCAAGGACAT | GTKDSTKDIPETPSKD (SEQ ID |
| | | CCCCGAGACCCCCTCCAAGGAC | NO: 1661) |
| | | (SEQ ID NO: 1816) | |
| | X10 Linker | TCCGGCTCCGAGACCCCCGGCACCTC | SGSETPGTSESATPES (SEQ ID |
| | | CGAGTCCGCCACCCCCGAGTCC | NO: 1601) |
| | | (SEQ ID NO: 1739) | |
| 3′ UTR | 3′ XBG | ACCAGCCTCAAGAACACCCGAATGGA | N/A |
| | | GTCTCTAAGCTACATAATACCAACTT | |
| | | ACACTTTACAAAATGTTGTCCCCCAA | |
| | | AATGTAGCCATTCGTATCTGCTCCTA | |
| | | ATAAAAAGAAAGTTTCTTCACATTCT | |
| | | (SEQ ID NO: 1684) | |
| | xr-RNA | TGTCAGCCCAGAACCCCACACGAGTT | N/A |
| | | TTGCCACTGCTAAGCTGTGAGGCAGT | |
| | | GCAGGCTGGGACAGCCGACCTCCAGG | |
| | | TTGCGAAAAACCTGGT (SEQ ID | |
| | | NO: 1740) | |
| polyA | pA1-4 | AAAAAAAAAAAATGGAAAAAAAAAAA | N/A |
| tail | | ACGGAAAAAAAAAAAAGGTAAAAAAA | |
| | | AAAAATATAAAAAAAAAAAACATAAA | |
| | | AAAAAAAAACGAAAAAAAAAAAACGT | |
| | | AAAAAAAAAAAACTCAAAAAAAAAAA | |
| | | AGATAAAAAAAAAAAACCTAAAAAAA | |
| | | AAAAATGTAAAAAAAAAAAAGGGAAA | |
| | | AAAAAAAAACGCAAAAAAAAAAAACA | |
| | | CAAAAAAAAAAAATGCAAAAAAAAAA | |
| | | AATCGAAAAAAAAAAAATCTAAAAAA | |
| | | AAAAAACGAAAAAAAAAAAACCCAAA | |
| | | AAAAAAAAAGACAAAAAAAAAAAATA | |
| | | GAAAAAAAAAAAGTTAAAAAAAAAAA | |
| | | ACTGAAAAAAAAAAAATTTAAAAAAA | |
| | | AAAAAT (SEQ ID NO: 1741) | |
| | pA100 | AAAAAAAAAAAAAAAAAAAAAAAAAA | N/A |
| | | AAAAAAAAAAAAAAAAAAAAAAAAAA | |
| | | AAAAAAAAAAAAAAAAAAAAAAAAAA | |
| | | AAAAAAAAAAAAAAAAAAAAAA | |
| | | (SEQ ID NO: 1742) | |
| hetero | EIcc | ATCGCCGCCCTGGAGAAGGAGATCGC | IAALEKEIAALEKENAALEWEIAAL |
| dimeri | | CGCCCTGGAGAAGGAGAACGCCGCCC | EK (SEQ ID NO: 1589) |
| zation | | TGGAGTGGGAGATCGCCGCCCTGGAG | |
| domain | | AAG (SEQ ID NO: 1588) | |
| | EIcc (with E at | GAGATCGCCGCCCTGGAGAAGGAGAT | EIAALEKEIAALEKENAALEWEIAA |
| | the N terminus) | CGCCGCCCTGGAGAAGGAGAACGCCG | LEK (SEQ ID NO: 1593) |
| | | CCCTGGAGTGGGAGATCGCCGCCCTG | |
| | | GAGAAG (SEQ ID NO: 1592) | |
| | KIcc | AAGATCGCCGCCCTGAAGGAGAAGAT | KIAALKEKIAALKEKNAALKWKIAA |
| | | CGCCGCCCTGAAGGAGAAGAACGCCG | LKE (SEQ ID NO: 1595) |
| | | CCCTGAAGTGGAAGATCGCCGCCCTG | |
| | | AAGGAG (SEQ ID NO: 1594) | |
| split | CfaN | TGCCTGTCCTACGACACCGAGATCCT | CLSYDTEILTVEYGFLPIGKIVEER |
| intein | | GACCGTGGAGTACGGCTTCCTGCCCA | IECTVYTVDKNGFVYTQPIAQWHNR |
| | | TCGGCAAGATCGTGGAGGAGCGGATC | GEQEVFEYCLEDGSIIRATKDHKFM |
| | | GAGTGCACCGTGTACACCGTGGACAA | TTDGQMLPIDEIFERGLDLKQVDGL |
| | | GAACGGCTTCGTGTACACCCAGCCCA | P (SEQ ID NO: 1744) |
| | | TCGCCCAGTGGCACAACCGCGGCGAG | |
| | | CAGGAGGTGTTCGAGTACTGCCTGGA | |
| | | GGACGGCTCCATCATCCGCGCCACCA | |
| | | AGGACCACAAGTTCATGACCACCGAC | |
| | | GGCCAGATGCTGCCCATCGACGAGAT | |
| | | CTTCGAGCGCGGCCTGGACCTGAAGC | |
| | | AGGTGGACGGCCTGCCC (SEQ ID | |
| | | NO: 1743) | |
| | CfaC | GTGAAGATCATCTCCCGCAAGTCCCT | VKIISRKSLGTQNVYDIGVEKDHNF |
| | | GGGCACCCAGAACGTGTACGACATCG | LLKNGLVASN (SEQ ID |
| | | GCGTGGAGAAGGACCACAACTTCCTG | NO: 1746) |
| | | CTGAAGAACGGCCTGGTGGCCTCCAA | |
| | | C (SEQ ID NO: 1745) | |
| | NpuN_flank | CGGGGCAAG | RGK |
| | NpuC_flank | TGCTGGGAG | CWE |
| | Cfa_flank | TGCTTCAAC | CFN |
| editing | Vpx | SEQ ID NO: 1468 | AAAMTDPRETVPPGNSGEETIGEAF |
| enhancers | | (See Table 6) | AWLNRTVEAINREAVNHLPRELIFQ |
| | | | VWQRSWRYWHDEQGMSESYTKYRYL |
| | | | CIIQKAVYMHVRKGCTCLGRGHGPG |
| | | | GWRPGPPPPPPPGLV (SEQ ID |
| | | | NO: 1469 |
| | FEN1 | SEQ ID NO: 1474 | MGIQGLAKLIADVAPSAIRENDIKS |
| | | (See Table 6) | YFGRKVAIDASMSIYQFLIAVRQGG |
| | | | DVLQNEEGETTSHLMGMFYRTIRMM |
| | | | ENGIKPVYVFDGKPPQLKSGELAKR |
| | | | SERRAEAEKQLQQAQAAGAEQEVEK |
| | | | FTKRLVKVTKQHNDECKHLLSLMGI |
| | | | PYLDAPSEAEASCAALVKAGKVYAA |
| | | | ATEDMDCLTFGSPVLMRHLTASEAK |
| | | | KLPIQEFHLSRILQELGLNQEQFVD |
| | | | LCILLGSDYCESIRGIGPKRAVDLI |
| | | | QKHKSIEEIVRRLDPNKYPVPENWL |
| | | | HKEAHQLFLEPEVLDPESVELKWSE |
| | | | PNEEELIKFMCGEKQFSEERIRSGV |
| | | | KRLSKSRQGSTQGRLDDFFKVTGSL |
| | | | SSAKRKEPEPKGSTKKKAKTGAAGK |
| | | | FKRGK (SEQ ID NO: 1475) |
| other | 3xFLAG tag | GACTACAAGGACGACGACGACAAGGA | DYKDDDDKDYKDDDDKDYKDDDDK |
| elements | | CTACAAGGACGACGACGACAAGGACT | (SEQ ID NO: 1748) |
| | | ACAAGGACGACGACGACAAG (SEQ | |
| | | ID NO: 1747) | |
| | HiBiT tag | GTGAGCGGCTGGCGGCTGTTCAAGAA | VSGWRLFKKIS (SEQ ID NO: |
| | | GATTAGC (SEQ ID NO: 1832) | 1833) |
| | P2A | GGCTCCGGCGCCACCAACTTCTCCCT | GSGATNFSLLKQAGDVEENPGP |
| | | GCTGAAGCAGGCCGGCGACGTGGAGG | (SEQ ID NO: 1750) |
| | | AGAACCCCGGCCCC (SEQ ID | |
| | | NO: 1749) | |
| | T2A | GGCTCCGGCGAGGGCCGGGGCTCCCT | GSGEGRGSLLTCGDVEENPGP |
| | | GCTGACCTGCGGCGACGTGGAGGAGA | (SEQ ID NO: 1752) |
| | | ACCCCGGCCCC (SEQ ID | |
| | | NO: 1751) | |

TABLE 4B
Exemplary nucleic caid (e.g., mRNA) constructs encoding nCas9-DNA Polymerase

| Exemplary | | mRNA regions from 5′ to 3′ | | | | | | | | | |
|---|---|---|---|---|---|---|---|---|---|---|---|
| nucleic acid | | | | | | | | Poly- | | | |
| (SEQ ID NO) | | | | | | Cas9 | | merase | | | |
| with | without | Cap | | | | coding | Linker | coding | Linker | | |
| a tag | a tag | analog | 5′UTR | Kozak | NLS | sequence | Region 1 | sequence | Region 2 | 3′UTR | Poly(A) |
| 2501 | 2586 | Cap0 | XBG | Kozak V1 | Nucleoplasmin | nCas9 | L42 | T5 Pol | L16-SV40- | XBG | pA1-4 |
| | | | | | | | | | cMyc | | |
| 2502 | 2587 | Cap1 | 4GApt1-XBG | Kozak V2 | Nucleoplasmin | nCas9 | L42(SV40) | T5 Pol | L16-SV40- | XBG | pA1-4 |
| | | | | | | | | | cMyc | | |
| N/A | 2588 | Cap1 | 4GApt1-XBG | Kozak V2 | Nucleoplasmin | nCas9 | L42(SV40) | T5 Pol | L16-SV40- | XBG | pA1-4 |
| | | | | | | | | | cMyc | | |
| N/A | 2589 | Cap1 | 4GApt1-XBG | Kozak V2 | Nucleoplasmin | nCas9 | L42(SV40) | T5 Pol | L16-SV40- | XBG | pA1-4 |
| | | | | | | | | | cMyc | | |
| N/A | 2590 | Cap1 | 4GApt1-XBG | Kozak V2 | Nucleoplasmin | nCas9 | L42(SV40) | T5 Pol | L16-SV40- | XBG | pA1-4 |
| | | | | | | | | | cMyc | | |
| 2506 | 2591 | Cap1 | 4GApt1-XBG | Kozak V1 | Nucleoplasmin | nCas9 | L42(SV40) | T5 Pol | X10-SV40 | XBG | pA1-4 |
| 2507 | 2592 | Cap1 | 4GApt1-XBG | Kozak V1 | Nucleoplasmin | nCas9 | GH5-SV40-GH5 | T5 Pol | X10-SV40 | XBG | pA1-4 |
| 2508 | 2593 | Cap1 | 4GApt1- | Kozak V1 | Nucleoplasmin | nCas9 | GH5-SV40-GH5 | T5 Pol | X10-SV40 | XBG | pA1-4 |
| | | | PABPv3-XBG | | | | | | | | |
| 2509 | 2594 | Cap1 | XBG | Kozak V1 | Nucleoplasmin | nCas9 | L42(SV40) | T5 Pol | L16-SV40- | XBG | pA1-4 |
| | | | | | | | | | cMyc | | |
| 2510 | 2595 | Cap1 | XBG | Kozak V1 | Nucleoplasmin | nCas9 | L42(SV40) | T5 Pol | L16-SV40- | XBG | pA100 |
| | | | | | | | | | cMyc | | |
| 2511 | 2596 | Cap1 | 4GApt1-XBG | Kozak V1 | Nucleoplasmin | nCas9 | L42(SV40) | T5 Pol | L16-SV40- | XBG | pA1-4 |
| | | | | | | | | | cMyc | | |
| 2512 | 2597 | Cap1 | XBG | Kozak V1 | Nucleoplasmin | nCas9 | L42(SV40) | T5 Pol | L16-SV40- | XBG- | pA1-4 |
| | | | | | | | | | cMyc | xrRNA | |
| 2513 | 2598 | Cap1 | 4GApt1-XBG | Kozak V1 | Nucleoplasmin | nCas9 | L42(SV40) | T5 Pol | L16-SV40- | XBG- | pA1-4 |
| | | | | | | | | | cMyc | xrRNA | |
| 2514 | 2599 | Cap1 | 4GApt1- | Kozak V1 | Nucleoplasmin | nCas9 | L42(SV40) | T5 Pol | L16-SV40- | XBG | pA1-4 |
| | | | PABPv3-XBG | | | | | | cMyc | | |
| 2515 | 2600 | Cap1 | 4EApt1-XBG | Kozak V1 | Nucleoplasmin | nCas9 | L42(SV40) | T5 Pol | L16-SV40- | XBG | pA1-4 |
| | | | | | | | | | cMyc | | |
| 2516 | 2601 | Cap1 | XBG | Kozak V1 | Nucleoplasmin | nCas9 | L42(SV40) | T5 Pol | L16-SV40- | XBG | pA1-4 |
| | | | | | | | | | cMyc | | |
| 2517 | 2602 | Cap1 | 4GApt1-XBG | Kozak V1 | Nucleoplasmin | nCas9 | L42(SV40) | T5 Pol | L16-SV40- | XBG | pA1-4 |
| | | | | | | | | | cMyc | | |
| 2518 | 2603 | Cap1 | XBG | Kozak V1 | Nucleoplasmin | nCas9 | L42(SV40) | T5 Pol | L16-SV40- | XBG- | pA1-4 |
| | | | | | | | | | cMyc | xrRNA | |
| 2519 | 2604 | Cap1 | 4GApt1-XBG | Kozak V1 | Nucleoplasmin | nCas9 | L42(SV40) | T5 Pol | L16-SV40- | XBG- | pA1-4 |
| | | | | | | | | | cMyc | xrRNA | |
| 2520 | 2605 | Cap1 | 4GApt1- | Kozak V1 | Nucleoplasmin | nCas9 | L42(SV40) | T5 Pol | L16-SV40- | XBG | pA1-4 |
| | | | PABPv3-XBG | | | | | | cMyc | | |
| 2521 | 2606 | Cap1 | 4EApt1-XBG | Kozak V1 | Nucleoplasmin | nCas9 | L42(SV40) | T5 Pol | L16-SV40- | XBG | pA1-4 |
| | | | | | | | | | cMyc | | |
| 2522 | 2607 | Cap1 | XBG | Kozak V1 | Nucleoplasmin | nCas9 | L42(SV40) | T5 Pol | L16-SV40- | XBG | pA1-4 |
| | | | | | | | | | cMyc | | |
| 2523 | 2608 | Cap1 | 4GApt1- | Kozak V1 | Nucleoplasmin | nCas9 | L42(SV40) | T5 Pol | L16-SV40- | XBG | pA1-4 |
| | | | PABPv3-XBG | | | | | | cMyc | | |
| 2524 | 2609 | Cap1 | XBG | Kozak V1 | Nucleoplasmin | nCas9 | L42(SV40) | T5 Pol | L16-SV40- | XBG | pA1-4 |
| | | | | | | | | | cMyc | | |
| 2525 | 2610 | Cap1 | 4GApt1- | Kozak V1 | Nucleoplasmin | nCas9 | L42(SV40) | T5 Pol | L16-SV40- | XBG | pA1-4 |
| | | | PABPv3-XBG | | | | | | cMyc | | |
| 2526 | 2611 | Cap1 | XBG | Kozak V1 | Nucleoplasmin | nCas9 | L42(SV40) | T5 Pol | X10-SV40- | XBG | pA1-4 |
| | | | | | | | | | cMyc | | |
| 2527 | 2612 | Cap1 | XBG | Kozak V1 | Nucleoplasmin | nCas9 | GH5-SV40-GH5 | T5 Pol | X10-SV40- | XBG | pA1-4 |
| | | | | | | | | | cMyc | | |
| 2528 | 2613 | Cap1 | 4GApt1-XBG | Kozak V2 | Nucleoplasmin | nCas9 | L42(SV40) | T5 Pol | L16-SV40- | XBG | pA1-4 |
| | | | | | | | | | cMyc-L16- | | |
| | | | | | | | | | EIcc | | |
| 2529 | 2614 | Cap1 | 4GApt1-XBG | Kozak V2 | Nucleoplasmin | nCas9 | L42(SV40) | T5 Pol | X10-SV40- | XBG | pA1-4 |
| | | | | | | | | | cMyc-X10- | | |
| | | | | | | | | | EIcc | | |
| 2530 | 2615 | Cap1 | 4GApt1-XBG | Kozak V2 | Nucleoplasmin | nCas9 | L42(SV40) | T5 Pol | X10-EIcc- | XBG | pA1-4 |
| | | | | | | | | | X10-SV40- | | |
| | | | | | | | | | cMyc | | |
| 2531 | 2616 | Cap1 | 4GApt1-XBG | Kozak V2 | Nucleoplasmin | nCas9 | 3xGH5 | T5 Pol | X10-SV40- | XBG | pA1-4 |
| | | | | | | | | | cMyc | | |
| 2532 | 2617 | Cap1 | 4GApt1-XBG | Kozak V2 | Nucleoplasmin | nCas9 | 3xGH5 | T5 Pol | X10-SV40- | XBG | pA1-4 |
| | | | | | | | | | cMyc-X10- | | |
| | | | | | | | | | Eicc | | |
| 2533 | 2618 | Cap1 | 4GApt1- | Kozak V1 | Nucleoplasmin | nCas9 | L42(SV40) | T5 Pol | X10-SV40 | XBG | pA1-4 |
| | | | PABPv3-XBG | | | | | | | | |
| 2534 | 2619 | Cap1 | 4GApt1- | Kozak V1 | Nucleoplasmin | nCas9 | GH5-SV40-GH5 | T5 Pol | X10-SV40 | XBG | pA1-4 |
| | | | PABPv3-XBG | | | | | | | | |
| 2535 | 2620 | Cap1 | 4GApt1-XBG | Kozak V1 | Nucleoplasmin | nCas9 | GH5-SV40-GH5 | T5 Pol | X10-SV40 | XBG | pA1-4 |
| 2536 | 2621 | Cap1 | XBG | Kozak V1 | Nucleoplasmin | nCas9 | L42(SV40) | T5 Pol | L16-SV40- | XBG | pA1-4 |
| | | | | | | | | | cMyc | | |
| 2537 | 2622 | Cap0 | XBG | Kozak V1 | bpSV40 | nCas9 | 2xSGGS-bpSV40- | PolK | L16-SV40 | XBG | pA1-4 |
| | | | | | | | 2xSGGS | | | | |
| 2538 | 2623 | Cap1 | 4GApt1-XBG | Kozak V1 | bpSV40 | nCas9 | GH5-SV40-GH5 | PolK | X10-SV40 | XBG | pA1-4 |
| 2539 | 2624 | Cap1 | 4GApt1- | Kozak V1 | bpSV40 | nCas9 | GH5-SV40-GH5 | PolK | X10-SV40 | XBG | pA1-4 |
| | | | PABPv3-XBG | | | | | | | | |
| 2540 | 2625 | Cap1 | 4GApt1-XBG | Kozak V2 | bpSV40 | nCas9 | 2xSGGS-bpSV40- | PolK | L16-SV40 | XBG | pA1-4 |
| | | | | | | | 2xSGGS | | | | |
| | 2626 | Cap1 | 4GApt1-XBG | Kozak V2 | bpSV40 | nCas9 | 2xSGGS-bpSV40- | PolK | L16-SV40 | XBG | pA1-4 |
| | | | | | | | 2xSGGS | | | | |
| | 2627 | Cap1 | 4GApt1-XBG | Kozak V2 | bpSV40 | nCas9 | 2xSGGS-bpSV40- | PolK | L16-SV40 | XBG | pA1-4 |
| | | | | | | | 2xSGGS | | | | |
| | 2628 | Cap1 | 4GApt1-XBG | Kozak V2 | bpSV40 | nCas9 | 2xSGGS-bpSV40- | PolK | L16-SV40 | XBG | pA1-4 |
| | | | | | | | 2xSGGS | | | | |
| 2544 | 2629 | Cap1 | 4GApt1-XBG | Kozak V1 | bpSV40 | nCas9 | 2xSGGS-bpSV40- | PolK | X10-SV40 | XBG | pA1-4 |
| | | | | | | | 2xSGGS | | | | |
| 2545 | 2630 | Cap1 | 4GApt1-XBG | Kozak V1 | bpSV40 | nCas9 | L42(SV40) | PolK | X10-SV40 | XBG | pA1-4 |
| 2546 | 2631 | Cap1 | XBG | Kozak V1 | bpSV40 | nCas9 | 2xSGGS-bpSV40- | PolK | L16-SV40 | XBG | pA1-4 |
| | | | | | | | 2xSGGS | | | | |
| 2547 | 2632 | Cap1 | XBG | Kozak V1 | bpSV40 | nCas9 | 2xSGGS-bpSV40- | PolK | L16-SV40 | XBG | pA100 |
| | | | | | | | 2xSGGS | | | | |
| 2548 | 2633 | Cap1 | 4GApt1-XBG | Kozak V1 | bpSV40 | nCas9 | 2xSGGS-bpSV40- | PolK | L16-SV40 | XBG | pA1-4 |
| | | | | | | | 2xSGGS | | | | |
| 2549 | 2634 | Cap1 | XBG | Kozak V1 | bpSV40 | nCas9 | 2xSGGS-bpSV40- | PolK | L16-SV40 | XBG- | pA1-4 |
| | | | | | | | 2xSGGS | | | xrRNA | |
| 2550 | 2635 | Cap1 | 4GApt1-XBG | Kozak V1 | bpSV40 | nCas9 | 2xSGGS-bpSV40- | PolK | L16-SV40 | XBG- | pA1-4 |
| | | | | | | | 2xSGGS | | | xrRNA | |
| 2551 | 2636 | Cap1 | 4EApt1-XBG | Kozak V1 | bpSV40 | nCas9 | 2xSGGS-bpSV40- | PolK | L16-SV40 | XBG | pA1-4 |
| | | | | | | | 2xSGGS | | | | |
| 2552 | 2637 | Cap1 | 4GApt1- | Kozak V1 | bpSV40 | nCas9 | 2xSGGS-bpSV40- | PolK | L16-SV40 | XBG | pA1-4 |
| | | | PABPv3-XBG | | | | 2xSGGS | | | | |
| 2553 | 2638 | Cap1 | XBG | Kozak V1 | bpSV40 | nCas9 | 2xSGGS-bpSV40- | PolK | L16-SV40 | XBG | pA1-4 |
| | | | | | | | 2xSGGS | | | | |
| 2554 | 2639 | Cap1 | 4GApt1-XBG | Kozak V1 | bpSV40 | nCas9 | 2xSGGS-bpSV40- | PolK | L16-SV40 | XBG | pA1-4 |
| | | | | | | | 2xSGGS | | | | |
| 2555 | 2640 | Cap1 | XBG | Kozak V1 | bpSV40 | nCas9 | 2xSGGS-bpSV40- | PolK | L16-SV40 | XBG- | pA1-4 |
| | | | | | | | 2xSGGS | | | xrRNA | |
| 2556 | 2641 | Cap1 | 4GApt1-XBG | Kozak V1 | bpSV40 | nCas9 | 2xSGGS-bpSV40- | PolK | L16-SV40 | XBG- | pA1-4 |
| | | | | | | | 2xSGGS | | | xrRNA | |
| 2557 | 2642 | Cap1 | 4GApt1- | Kozak V1 | bpSV40 | nCas9 | 2xSGGS-bpSV40- | PolK | L16-SV40 | XBG | pA1-4 |
| | | | PABPv3-XBG | | | | 2xSGGS | | | | |
| 2558 | 2643 | Cap1 | 4EApt1-XBG | Kozak V1 | bpSV40 | nCas9 | 2xSGGS-bpSV40- | PolK | L16-SV40 | XBG | pA1-4 |
| | | | | | | | 2xSGGS | | | | |
| 2559 | 2644 | Cap1 | XBG | Kozak V1 | bpSV40 | nCas9 | 2xSGGS-bpSV40- | PolK | L16-SV40 | XBG | pA1-4 |
| | | | | | | | 2xSGGS | | | | |
| 2560 | 2645 | Cap1 | 4GApt1- | Kozak V1 | bpSV40 | nCas9 | 2xSGGS-bpSV40- | PolK | L16-SV40 | XBG | pA1-4 |
| | | | PABPv3-XBG | | | | 2xSGGS | | | | |
| 2561 | 2646 | Cap1 | XBG | Kozak V1 | bpSV40 | nCas9 | 2xSGGS-bpSV40- | PolK | L16-SV40 | XBG | pA1-4 |
| | | | | | | | 2xSGGS | | | | |
| 2562 | 2647 | Cap1 | 4GApt1- | Kozak V1 | bpSV40 | nCas9 | 2xSGGS-bpSV40- | PolK | L16-SV40 | XBG | pA1-4 |
| | | | PABPv3-XBG | | | | 2xSGGS | | | | |
| 2563 | 2648 | Cap1 | 4GApt1- | Kozak V1 | bpSV40 | nCas9 | 2xSGGS-bpSV40- | PolK | X10-SV40 | XBG | pA1-4 |
| | | | PABPv3-XBG | | | | 2xSGGS | | | | |
| 2564 | 2649 | Cap1 | 4GApt1- | Kozak V1 | bpSV40 | nCas9 | L42(SV40) | PolK | X10-SV40 | XBG | pA1-4 |
| | | | PABPv3-XBG | | | | | | | | |
| 2565 | 2650 | Cap1 | 4GApt1- | Kozak V1 | bpSV40 | nCas9 | GH5-SV40-GH5 | PolK | X10-SV40 | XBG | pA1-4 |
| | | | PABPv3-XBG | | | | | | | | |
| 2566 | 2651 | Cap1 | 4GApt1-XBG | Kozak V1 | bpSV40 | nCas9 | GH5-SV40-GH5 | PolK | X10-SV40 | XBG | pA1-4 |
| 2567 | 2652 | Cap1 | XBG | Kozak V1 | bpSV40 | nCas9 | 2xSGGS-bpSV40- | PolK | L16-SV40 | XBG | pA1-4 |
| | | | | | | | 2xSGGS | | | | |
| 2568 | 2653 | Cap1 | 4GApt1-XBG | Kozak V1 | bpSV41 | nCas9 | 2xSGGS-bpSV40- | PolK | X10-EIcc- | XBG | pA1-4 |
| | | | | | | | 2xSGGS | | X10-SV40 | | |
| 2569 | 2654 | Cap1 | XBG | Kozak V1 | bpSV42 | nCas9 | 2xSGGS-bpSV40- | PolK | L16-SV40 | XBG | pA1-4 |
| | | | | | | | 2xSGGS | | | | |
| 2570 | 2655 | Cap1 | XBG | Kozak V1 | bpSV43 | nCas9 | 2xSGGS-bpSV40- | PolK | L16-SV40 | XBG | pA1-4 |
| | | | | | | | 2xSGGS | | | | |
| 2571 | 2656 | Cap1 | XBG | Kozak V1 | bpSV40 | nCas9 | 2xSGGS-bpSV40- | PolK | L16-SV40 | XBG | pA1-4 |
| | | | | | | | 2xSGGS | | | | |
| 2572 | 2657 | Cap1 | XBG | Kozak V1 | bpSV40 | nCas9 | 2xSGGS-bpSV40- | PolK | L16-SV40 | XBG | pA1-4 |
| | | | | | | | 2xSGGS | | | | |
| 2573 | 2658 | Cap1 | XBG | Kozak V1 | bpSV40 | nCas9 | 2xSGGS-bpSV40- | PolK | L16-SV40 | XBG | pA1-4 |
| | | | | | | | 2xSGGS | | | | |
| 2574 | 2659 | Cap1 | XBG | Kozak V1 | bpSV40 | nCas9 | 2xSGGS-bpSV40- | PolK | L16-SV40 | XBG | pA1-4 |
| | | | | | | | 2xSGGS | | | | |
| 2575 | 2660 | Cap1 | XBG | Kozak V1 | bpSV40 | nCas9 | 2xSGGS-bpSV40- | PolK | L16-SV40 | XBG | pA1-4 |
| | | | | | | | 2xSGGS | | | | |
| 2576 | 2661 | Cap1 | XBG | Kozak V1 | bpSV40 | nCas9 | 2xSGGS-bpSV40- | PolK | L16-SV40 | XBG | pA1-4 |
| | | | | | | | 2xSGGS | | | | |
| 2577 | 2662 | Cap1 | XBG | Kozak V1 | bpSV40 | nCas9 | 2xSGGS-bpSV40- | PolK | X10-SV40 | XBG | pA1-4 |
| | | | | | | | 2xSGGS | | | | |
| 2578 | 2663 | Cap1 | XBG | Kozak V1 | bpSV40 | nCas9 | L42 | PolK | X10-SV40 | XBG | pA1-4 |
| 2579 | 2664 | Cap1 | XBG | Kozak V1 | bpSV40 | nCas9 | GH5-SV40-GH5 | PolK | X10-SV40 | XBG | pA1-4 |
| 2580 | 2665 | Cap1 | 4GApt1-XBG | Kozak V2 | bpSV40 | nCas9 | 2xSGGS-bpSV40- | PolK | L16-SV40- | XBG | pA1-4 |
| | | | | | | | 2xSGGS | | L16-Eicc | | |
| 2581 | 2666 | Cap1 | 4GApt1-XBG | Kozak V2 | bpSV40 | nCas9 | 2xSGGS-bpSV40- | PolK | X10-SV40- | XBG | pA1-4 |
| | | | | | | | 2xSGGS | | X10-EIcc | | |
| 2582 | 2667 | Cap1 | 4GApt1-XBG | Kozak V2 | bpSV40 | nCas9 | 2xSGGS-bpSV40- | PolK | X10-EIcc- | XBG | pA1-4 |
| | | | | | | | 2xSGGS | | X10-SV40 | | |
| 2583 | 2668 | Cap1 | 4GApt1-XBG | Kozak V2 | bpSV40 | nCas9 | 3xGH5 | PolK | X10-EIcc- | XBG | pA1-4 |
| | | | | | | | | | X10-SV40 | | |
| 2584 | 2669 | Cap1 | 4GApt1-XBG | Kozak V2 | bpSV40 | nCas9 | 3xGH5 | PolK | X10-EIcc- | XBG | pA1-4 |
| | | | | | | | | | X10-SV40 | | |
| 2585 | 2670 | Cap1 | 4GApt1-XBG | Kozak V2 | bpSV40 | nCas9 | 3xGH5 | PolK | X10-SV40 | XBG | pA1-4 |

*** Linker region 1 may optionally comprise a NLS. Linker region 2 may optionally comprise a NLS or a heterodimerzation domain. Linker region 3 may optionally comprise a NLS or a heterodimerzation domain.

TABLE 4C
Exemplary nucleic acid (e.g., mRNA) constructs encoding N-intein and C-intein constructs described herein

| Exemplary | mRNA region from 5′ to 3′ | | | | | | | | | |
|---|---|---|---|---|---|---|---|---|---|---|
| nucleic | | | | | coding | | coding | | | |
| acid (SEQ | Cap | | | Linker | sequence | Linker | sequence | Linker | | |
| ID NO) | analog | 5′UTR | Kozak | region 1 | 1 | region 2 | 2 | region 3 | 3′UTR | Poly(A) |
| 3001 | Cap | 4GApt1- | Kozak V2 | CfaC-Cfa_flank-SGGS- | T5 Pol | L16-SV40-cMyc | N/A | N/A | XBG | pA1-4 |
| | 1 | XBG | | bpSV40-2xSGGS | | | | | | |
| 3002 | Cap | 4GApt1- | Kozak V2 | CfaC-Cfa_flank-SGGS- | T5 Pol | L16-SV40-cMyc- | N/A | N/A | XBG | pA1-4 |
| | 1 | XBG | | bpSV40-2xSGGS | | L16-EIcc | | | | |
| 3003 | Cap | 4GApt1- | Kozak V2 | CfaC-Cfa_flank-SGGS- | T5 Pol | L16-SV40-cMyc- | N/A | N/A | XBG | pA1-4 |
| | 1 | XBG | | bpSV40-2xSGGS | | X10-EIcc | | | | |
| 3004 | Cap | 4GApt1- | Kozak V2 | CfaC-Cfa_flank-SGGS- | PolK | L16-SV40-cMyc- | N/A | N/A | XBG | pA1-4 |
| | 1 | XBG | | bpSV40-2xSGGS | | L16-EIcc | | | | |
| 3005 | Cap | 4GApt1- | Kozak V2 | CfaC-Cfa_flank-SGGS- | PolK | L16-SV40-cMyc- | N/A | N/A | XBG | pA1-4 |
| | 1 | XBG | | bpSV40-2xSGGS | | X10-EIcc | | | | |
| 3006 | Cap | 4GApt1- | Kozak V2 | Nucleoplasmin-X10- | nCas9 | SGGS- | N/A | N/A | XBG | pA1-4 |
| | 1 | XBG | | HMGB1dT-2xGH5 | | NpuN_flank-CfaN | | | | |
| 3007 | Cap | 4GApt1- | Kozak V2 | Nucleoplasmin- | nCas9 | SGGS- | N/A | N/A | XBG | pA1-4 |
| | 1 | XBG | | HMGN1-2xGH5 | | NpuN_flank-CfaN | | | | |
| 3008 | Cap | 4GApt1- | Kozak V2 | Nucleoplasmin | nCas9 | SGGS- | N/A | N/A | XBG | pA1-4 |
| | 1 | XBG | | | | NpuN_flank-CfaN | | | | |
| 3009 | Cap | 4GApt1- | Kozak V2 | CfaC-Cfa_flank-SGGS- | PolK | L16-SV40-cMyc | N/A | N/A | XBG | pA1-4 |
| | 1 | XBG | | bpSV40-2xSGGS | | | | | | |
| 3010 | Cap | 4GApt1- | Kozak V2 | N/A | nCas9 | 2xSGGS-bpSV40- | N/A | N/A | XBG | pA1-4 |
| | 1 | XBG | | | | SGGS- | | | | |
| | | | | | | NpuN_flank-CfaN | | | | |
| 3011 | Cap | 4GApt1- | Kozak V2 | CfaC-Cfa_flank-SGGS | PolK | L16-SV40-cMyc | N/A | N/A | XBG | pA1-4 |
| | 1 | XBG | | | | | | | | |
| 3012 | Cap | 4GApt1- | Kozak V2 | Nucleoplasmin | nCas9 | SGGS- | N/A | N/A | XBG | pA1-4 |
| | 1 | XBG | | | | NpuN_flank-NpuN | | | | |
| 3013 | Cap | 4GApt1- | Kozak V2 | NpuC-NpuC_flank- | PolK | L16-SV40-cMyc | N/A | N/A | XBG | pA1-4 |
| | 1 | XBG | | SGGS-bpSV40- | | | | | | |
| | | | | 2xSGGS | | | | | | |
| 3014 | Cap | 4GApt1- | Kozak V2 | CfaC-Cfa_flank-SGGS- | PolK | L16-SV40-cMyc- | N/A | N/A | XBG | pA1-4 |
| | 1 | XBG | | bpSV40-2xSGGS | | T2A-Vpx | | | | |
| 3015 | Cap | 4GApt1- | Kozak V2 | CfaC-Cfa_flank- | T5 Pol | L16-SV40-cMyc- | N/A | N/A | XBG | pA1-4 |
| | 1 | XBG | | L42(SV40) | | T2A-Vpx | | | | |
| 3016 | Cap | 4GApt1- | Kozak V2 | CfaC-Cfa_flank- | T5 Pol | L16-SV40-cMyc | N/A | N/A | XBG | pA1-4 |
| | 1 | XBG | | L42(SV40) | | | | | | |
| 3017 | Cap | 4GApt1- | Kozak V2 | SGGS-Nucleoplasmin- | nCas9* | NpuN_flank-CfaN | N/A | N/A | XBG | pA1-4 |
| | 1 | XBG | | GGSGGG | | | | | | |
| 3018 | Cap | 4GApt1- | Kozak V2 | Nucleoplasmin-X10- | nCas9* | NpuN_flank-CfaN | N/A | N/A | XBG | pA1-4 |
| | 1 | XBG | | HMGB1dT-2xGH5 | | | | | | |
| 3019 | Cap | 4GApt1- | Kozak V2 | CfaC-Cfa_flank | nCas9** | 2xSGGS-bpSV40- | PolK | L16-SV40- | XBG | pA1-4 |
| | 1 | XBG | | | | 2xSGGS | | cMyc | | |
| 3020 | Cap | 4GApt1- | Kozak V2 | CfaC-Cfa_flank | nCas9** | 2xSGGS-bpSV40- | PolK | L16-SV40- | XBG | pA1-4 |
| | 1 | XBG | | | | 2xSGGS | | cMyc-X10- | | |
| | | | | | | | | Eicc | | |
| 3021 | Cap | 4GApt1- | Kozak V2 | CfaC-Cfa_flank | nCas9** | 2xSGGS-bpSV40- | T5 Pol | L16-SV40- | XBG | pA1-4 |
| | 1 | XBG | | | | 2xSGGS | | cMyc | | |
| 3022 | Cap | 4GApt1- | Kozak V2 | CfaC-Cfa_flank | nCas9** | 2xSGGS-bpSV40- | T5 Pol | L16-SV40- | XBG | pA1-4 |
| | 1 | XBG | | | | 2xSGGS | | cMyc-X10- | | |
| | | | | | | | | Eicc | | |

*The Cas9 in this construct is SpyCas9(2-713) and is encoded by the sequence of SEQ ID NO:
**The Cas9 in this construct is SpyCas9(714-1368)
*** Linker region 1 may optionally comprise NLS, chromatin remodeler, or split intein. Linker region 2 may optionally comprise NLS, split intein, editing enhancer, or coiled coil. Linker region 3 may optionally comprise NLS, split intein, editing enhancer, or coiled coil.

TABLE 4D
Exemplary nucleic caid (e.g., mRNA) constructs encoding one or more enhancer for template editing

| Exemplary mRNA sequence | | | | | | | | |
|---|---|---|---|---|---|---|---|---|
| SEQ ID NO | | mRNA regions from 5′ to 3′ | | | | | | |
| with | Without | Cap | | | Enhancer | | | |
| a tag | a tag | analog | 5′UTR | Kozak | coding sequence | Linker region* | 3′UTR | Poly(A) |
| 3501 | 3516 | Cap0 | XBG | Kozak V2 | FEN1 | L14-SV40 | XBG | pA1-4 |
| 3502 | 3517 | Cap1 | 4GApt1-XBG | Kozak V2 | FEN1 | N/A | XBG | pA1-4 |
| 3503 | 3518 | Cap1 | XBG | Kozak V2 | FEN1 | L14-SV40 | XBG | pA1-4 |
| 3504 | 3519 | Cap1 | XBG | Kozak V2 | FEN1 | L14-SV40 | XBG | pA100 |
| 3505 | 3520 | Cap1 | 4GApt1-XBG | Kozak V2 | FEN1 | L14-SV40 | XBG | pA1-4 |
| 3506 | 3521 | Cap1 | XBG | Kozak V2 | FEN1 | L14-SV40 | XBG-xrRNA | pA1-4 |
| 3507 | 3522 | Cap1 | 4GApt1-XBG | Kozak V2 | FEN1 | L14-SV40 | XBG-xrRNA | pA1-4 |
| 3508 | 3523 | Cap1 | 4GApt1-XBG | Kozak V2 | Vpx-P2A-FEN1 | KIcc | XBG | pA1-4 |
| 3509 | 3524 | Cap1 | 4GApt1-XBG | Kozak V2 | Vpx-P2A-FEN1 | KIcc | XBG | pA1-4 |
| 3510 | 3525 | Cap1 | 4GApt1-XBG | Kozak V2 | FEN1 | KIcc | XBG | pA1-4 |
| 3511 | 3526 | Cap1 | 4GApt1-XBG | Kozak V2 | Vpx-X10-FEN1 | KIcc | XBG | pA1-4 |
| 3512 | 3527 | Cap1 | 4GApt1-XBG | Kozak V2 | Vpx-X10-FEN1 | KIcc | XBG | pA1-4 |
| 3513 | 3528 | Cap1 | 4GApt1-XBG | Kozak V2 | FEN1 | KIcc-P2A-3xFLAG-Vpx | XBG | pA1-4 |
| 3514 | 3529 | Cap1 | 4GApt1-XBG | Kozak V2 | FEN1 | KIcc-P2A-Vpx | XBG | pA1-4 |
| 3515 | 3530 | Cap1 | 4GApt1-XBG | Kozak V2 | Vpx | N/A | XBG | pA1-4 |

*Linker region may optionally comprise an NLS, heterodimerization domain, P2A.

### Methods of Use

In some embodiments, any one or more of the SpyCas9 nickase or a nucleic acid encoding the same, DNA-dependent DNA polymerase or a nucleic acid encoding the same, or template guide RNA for template-based genome editing, compositions, or pharmaceutical formulations described herein is for use in preparing a medicament for treating or preventing a disease or disorder in a subject.

In some embodiments, the present disclosure comprises a method of treating or preventing a disease or disorder in subject comprising administering any one or more of the SpyCas9 nickase or a nucleic acid encoding the same, DNA-dependent DNA polymerase or a nucleic acid encoding the same, or template guide RNA for template-based genome editing, compositions, or pharmaceutical formulations described herein.

In some embodiments, the present disclosure comprises a method or use of modifying a target DNA comprising, administering or delivering any one or more of the SpyCas9 nickase or a nucleic acid encoding the same, DNA-dependent DNA polymerase or a nucleic acid encoding the same, or template guide RNA for template-based genome editing, compositions, or pharmaceutical formulations described herein.

In some embodiments, the present disclosure comprises a method or use for modulation of a target gene comprising, administering or delivering any one or more of the SpyCas9 nickase or a nucleic acid encoding the same, DNA-dependent DNA polymerase or a nucleic acid encoding the same, or template guide RNA for template-based genome editing, compositions, or pharmaceutical formulations described herein. In some embodiments, the modulation is editing of the target gene. In some embodiments, the modulation is a change in expression of the protein encoded by the target gene. As used herein, “genome editing,” “gene editing,” or “genetic modification” is a change at the DNA sequence, e.g., induced by a Cas9 nickase, DNA-dependent DNA polymerase, and template guide RNA, that results in a change of expression of an mRNA or protein. A gene editing or genetic modification may comprise an insertion, deletion, or substitution (substitution of one or more bases, e.g., a point mutation such as C-to-T, a transition, a transversion, or a larger substitution such as GAGG-to-CACT), typically within a defined sequence or genomic locus. A genetic modification changes the nucleic acid sequence of the DNA. A genetic modification may be at a single nucleotide position. A genetic modification may be at multiple nucleotides, e.g., 2, 3, 4, 5 or more nucleotides, typically in close proximity to each other, e.g, consecutive nucleotides.

In some embodiments, the method or use results in template-based genome editing. In some embodiments, the method or use results in a single-strand break within the target gene. In some embodiments, the method or use further comprises delivering to the cell a polynucleotide comprising a 3′ extension, wherein the DRS of the 3′ extension base-pairs with the 3′ end of the flap generated at the 5′ end of a nick by the single-strand break, thereby allowing the 3′ end of the flap to serve as a primer for DNA synthesis by DNA-dependent DNA polymerase, wherein the template sequence of the 3′ extension serves as a template for DNA synthesis. In some embodiments, template-based genome editing results in edits comprising an insertion, deletion, or substitution in a nucleotide sequence. In some embodiments, template-based genome editing results in an insertion and a substitution in a nucleotide sequence. In some embodiments, template-based genome editing results in a deletion and a substitution in a nucleotide sequence. In some embodiments, template-based genome editing results in an insertion and a deletion in a nucleotide sequence.

In some embodiments, the template sequence comprises an insertion sequence relative to the corresponding genomic sequence. In some embodiments, the template sequence comprises an insertion sequence of at least one nucleotide. In some embodiments, the template sequence comprises an insertion sequence of 1-40 nucleotides in length. In some embodiments, the template sequence comprises an insertion sequence of at least 1, 2, 3, 4, 5, 6, 7, 8, 9, or 10 nucleotides in length. In some embodiments, the template sequence comprises an insertion sequence of at least 1, 5, 10, 15, 20, 25, 30, 35, 40, 45, 50, 55, 60, 65, 70, 75, 80, 85, 90, 95, or 100 nucleotides in length. In some embodiments, the template sequence comprises an insertion sequence of 1-100 or 5-100 nucleotides in length. In some embodiments, the template sequence comprises an insertion sequence of at least 100 nucleotides in length. In some embodiments, the template sequence comprises an insertion sequence of at least 100, 200, 300, 400, 500, 600, 700, 800, 900, or 1000 nucleotides in length. In some embodiments, the template sequence comprises an insertion sequence of 1-1000 nucleotides in length. In some embodiments, the template sequence comprises an insertion sequence of 500, 1000, 3500, 5000, or 10,000 nucleotides in length.

In some embodiments, the template sequence comprises a sequence for directing a deletion relative to the corresponding genomic sequence. In some embodiments, the template sequence comprises a sequence for directing a deletion of at least one nucleotide. In some embodiments, the template sequence comprises a sequence for directing a deletion of 1-40 nucleotides in length. In some embodiments, the template sequence comprises a sequence for directing a deletion of at least 1, 2, 3, 4, 5, 6, 7, 8, 9, or 10 nucleotides in length. In some embodiments, the template sequence comprises a sequence for directing a deletion of at least 1, 5, 10, 15, 20, 25, 30, 35, 40, 45, 50, 55, 60, 65, 70, 75, 80, 85, 90, 95, or 100 nucleotides in length. In some embodiments, the template sequence comprises a sequence for directing a deletion of at least 100 nucleotides in length. In some embodiments, the template sequence comprises a sequence for directing a deletion of at least 100, 200, 300, 400, 500, 600, 700, 800, 900, or 1000 nucleotides in length.

In some embodiments, the template sequence comprises a sequence for directing a deletion of at least 1000, 2000, 3000, 4000, 5000, 6000, 7000, 8000, 9000, or 10,000 nucleotides in length. In some embodiments, the template sequence comprises a sequence for directing a deletion of 1-10,000 nucleotides in length.

In some embodiments, the length of the templated substitution in nucleotide sequence is at least 1 nucleotide.

In some embodiments, the insertion, deletion, or substitution of nucleotides in a target gene leads to a frameshift mutation or premature stop codon that results in a non-functional protein. In some embodiments, the insertion, deletion, or substitution of nucleotides in a target gene corrects a frameshift mutation or removes a premature stop codon, restoring protein expression or function. In some embodiments, the insertion, deletion, or substitution of nucleotides in a target gene leads to a knockdown or elimination of target gene expression. In some embodiments, the insertion, deletion, or substitution of nucleotides in a target gene leads to an increase in target gene expression. The gene editing typically occurs within or adjacent to the portion of the target gene with which the spacer sequence forms a duplex.

In some embodiments, the method or use results in gene modulation. In some embodiments, the gene modulation is an increase or decrease in gene expression. In some embodiments, the method or use results in increased or decreased expression of the protein encoded by the target gene.

The efficacy of (a) a SpyCas9 nickase or a nucleic acid encoding the same, (b) a DNA-dependent DNA polymerase or a nucleic acid encoding the same, and (c) a template guide RNA for template-based genome editing can be tested in vitro and in vivo. In some embodiments, the present disclosure comprises the SpyCas9 nickase or a nucleic acid encoding the same, DNA-dependent DNA polymerase or a nucleic acid encoding the same, template guide RNA for template-based genome editing, compositions, or pharmaceutical formulations described herein, wherein the template guide RNA results in gene modulation when provided to a cell together with a Cas nuclease, e.g., SpyCas9 or mRNA encoding SpyCas9 and DNA-dependent DNA polymerase, e.g., a DNA-dependent DNA polymerase or mRNA encoding the DNA-dependent DNA polymerase. In some embodiments, the efficacy of guide RNA can be measured in vitro or in vivo.

In some embodiments, the efficiency of a SpyCas9 nickase, DNA-dependent DNA polymerase, and template guide RNA in increasing or decreasing target protein expression is determined by measuring the amount of target protein.

In some embodiments, the efficiency of editing with a specific SpyCas9 nickase, DNA-dependent DNA polymerase, and template guide RNA is determined by the editing present at the target location in the genome following delivery of the SpyCas9 nickase, DNA-dependent DNA polymerase, and template guide RNA. In some embodiments, the efficiency of editing with a specific SpyCas9 nickase, DNA-dependent DNA polymerase, and template guide RNA is measured by next-generation sequencing (NGS). In some embodiments, the editing percentage of the target sequence of interest is determined. In some embodiments, the total number of sequence reads with templated sequence alterations, e.g., insertions, deletions, or substitutions of nucleotides into the target sequence of interest over the total number of sequence reads is measured following delivery of a SpyCas9 nickase, DNA-dependent DNA polymerase, and template guide RNA. In some embodiments, the editing percentage is calculated including sequence reads with alterations in addition to the templated sequence alterations. In some embodiments, the editing percentage is calculated not including sequence reads with alterations in addition to the templated sequence alterations, i.e., perfect edits.

In some embodiments, the efficiency of editing with a specific SpyCas9 nickase, DNA-dependent DNA polymerase, and template guide RNA is measured by the presence of sequence alterations, e.g., insertions, deletions, or substitutions introduced by successful template-based gene editing. In some embodiments, activity of a SpyCas9 nickase, DNA-dependent DNA polymerase, and template guide RNA is tested in biochemical assays. In some embodiments, activity of a SpyCas9 nickase, DNA-dependent DNA polymerase, and template guide RNA is tested in primary cells, e.g., primary hepatocytes.

In some embodiments, the activity of SpyCas9 nickase, DNA-dependent DNA polymerase, and template guide RNA is measured after in vivo dosing of LNPs comprising a SpyCas9 nickase or nucleic acid encoding a SpyCas9 nickase, DNA-dependent DNA polymerase or nucleic acid encoding a DNA-dependent DNA polymerase, and template guide RNA.

In some embodiments, in vivo efficacy of a SpyCas9 nickase, DNA-dependent DNA polymerase, and template guide RNA, or composition provided herein is determined by editing efficacy measured in DNA extracted from tissue (e.g., liver tissue) after administration of a SpyCas9 nickase or nucleic acid encoding a SpyCas9 nickase, DNA-dependent DNA polymerase or nucleic acid encoding a DNA-dependent DNA polymerase, and template guide RNA.

In some embodiments, template-based genome editing results in at least 5%, 10%, 15%, 20%, 25%, 30%, 35%, 40%, 45%, optionally at least 50%, 55%, 60%, 65%, optionally at least 70%, 75%, 80%, 90%, 95% templated edits as a percent of total edits in the target nucleic acid.

In some embodiments, the cell is a liver cell, e.g., a hepatocyte, an immune cell, muscle cell, or nerve cell. In some embodiments, the immune cell is a T cell. In some embodiments, the cell is a hepatocyte and at least 5% of the cells in the cell population comprise a desired genome edit in the target nucleic acid. In some embodiments, the hepatocyte is in vivo. In some embodiments, the cell is an immune cell (T cell) and at least 50% of the cells in the cell population comprise a desired genome edit in the target nucleic acid.

In some embodiments, the method further comprises a nicking guide RNA (ngRNA). In some embodiments, the nicking guide RNA comprises a sequence of any one of SEQ ID NOs: 1-39, 101-130, 201-218, 225-231, 236, 240-273, 300-318, 326-348, and 375-383 or any one of the sequences listed in Tables 1A-1D. In some embodiments, the method further comprises a nicking guide RNA (ngRNA). In some embodiments, the nicking guide RNA comprises a sequence of any one of SEQ ID NOs: 1-39, 101-130, 201-218, 225-273, 274-276, 300-318, 325, 326-349, 372-383, and 431-433 or any one of the sequences listed in Tables 1A-1D. In some embodiments, the nicking guide RNA comprises a sequence of any one of SEQ ID NOs: 1-28, 35, 38-39, 101-120, 129-130, 201-218, 225-250, 269, 272-273, 274-276, 301-318, 325, 326-334, 347-349, 372-374, and 431-433. In some embodiments, the nicking guide RNA comprises a modification pattern of any one of SEQ ID NOs: 201-218, 225-231, 236, 240-273, 274, 300-318, 326-348, and 375-383 or any one of the sequences listed in Tables 1C-1D. In some embodiments, the nicking guide RNA comprises a modification pattern of any one of SEQ ID NOs: 201-218, 225-273, 274-276, 300-318, 325, 326-349, 372-383, and 431-433 or any one of the sequences listed in Tables 1C-1D. In some embodiments, the nicking guide RNA comprises a modification pattern of any one of SEQ ID NOs: 201-218, 225-250, 269, 272-273, 301-318, 326-334, 347-349, 372-374, and 431-433. In some embodiments, the scaffold of the nicking guide RNA comprises a modification pattern of nucleotide 21 to nucleotide at 3′ of any one of SEQ ID NOs: 301-318, 326-348, and 375-383 or any one of the sequences listed in Table ID. In some embodiments, the scaffold of the nicking guide RNA comprises a modification pattern of nucleotide 21 to nucleotide at 3′ of any one of SEQ ID NOs: 301-318, 326-349, 372-383, and 431-433 or any one of the sequences listed in Table 1D. In some embodiments, the scaffold of the nicking guide RNA comprises a modification pattern of nucleotide 21 to nucleotide at 3′ of any one of SEQ ID NOs: 301-318, 326-334, 347-349, 372-374, and 431-433. In some embodiments, the nicking guide RNA comprises a sequence of any one of SEQ ID NOs: 1-28, 101-120, 201-251, 301-334, or 350-372. In some embodiments, the nicking guide RNA comprises a sequence of any one of SEQ ID NOs: 1-28, 35, 38-39, 101-120, 129-130, 201-218, 225-250, 269, 272-273, 274-276, 301-318, 325, 326-334, 347-349, 372-374, or 431-433. In some embodiments, the nicking guide RNA comprises a modification pattern of any one of SEQ ID NOs: 201-251, 301-334, or 350-372. In some embodiments, the nicking guide RNA comprises a modification pattern of any one of SEQ ID NOs: 201-218, 225-250, 269, 272-273, 274-276, 301-318, 325, 326-334, 347-349, 372-374, or 431-433. In some embodiments, the scaffold of the nicking guide RNA comprises a modification pattern of nucleotide 21 to the nucleotide at 3′ of any one of SEQ ID NOs: 401-529. In some embodiments, the ngRNA comprises a spacer that is complementary to the non-target strand of the DNA duplex target nucleic acid. In some embodiments, the ngRNA comprises a spacer that hybridizes to a sequence such that the nick site of the ngRNA is within 200 nucleotides 5′ or 3′ from the nick site of the template guide RNA on the same strand as the first region. In some embodiments, the nick site of the ngRNA is about 20-200 nucleotides 5′ or 3′ from the nick site of the template guide RNA and on the same strand as the first region. In some embodiments, the nick site of the ngRNA is within the target site of the spacer. In some embodiments, the nick site of the ngRNA is outside the target site of the spacer.

In some embodiments, the method further comprises an enhancer of template-based genome editing. In some embodiments, the enhancer is an inhibitor of deoxynucleotide triphosphohydrolase. In some embodiments, wherein the enhancer is an inhibitor of deoxynucleotide triphosphohydrolase, the deoxynucleotide triphosphohydrolase comprises SAM domain and HD domain-containing protein 1 (SAMHD1). In some embodiments, inhibiting SAMHD1 comprises contacting the SAMHD1 with a Vpx, BGLF4, M97, or KSHV ORF36 protein or expressing the Vpx, BGLF4, M97, or KSHV ORF36 protein in a cell. In some embodiments, inhibiting SAMHD1 comprises contacting an mRNA encoding the SAMHD1 with a microRNA, shRNA, or siRNA that hybridizes to the mRNA, or expressing the microRNA, shRNA, or siRNA in a cell. In some embodiments, inhibiting SAMHD1 comprises contacting the SAMHD1 with a small molecule SAMHD1 inhibitor.

In some embodiments, the enhancer is a Vpx protein, BGLF4 protein, M97 protein, or KSHV ORF36 protein. In some embodiments, the enhancer is a Vpx protein. In some embodiments, the Vpx protein comprises an amino acid sequence that has at least 90%, 91%, 92%, 93%, 94%, 95%, 96%, 97%, 98%, 99%, or 100% identity to any one of SEQ ID NOs: 1464, 1469, and 1503-1505. In some embodiments, the Vpx protein is encoded by a nucleotide sequence that has at least 90%, 91%, 92%, 93%, 94%, 95%, 96%, 97%, 98%, 99%, or 100% identity to SEQ ID NO: 1462, 1463, 1468, 1470, 1499, 1501, 1502, 1521, or 1585, or 3530. In some embodiments, the enhancer is BGLF4 protein. In some embodiments, the BGLF4 comprises an amino acid sequence that has at least 90%, 91%, 92%, 93%, 94%, 95%, 96%, 97%, 98%, 99%, or 100% identity to SEQ ID NO: 1517 or 1518. In some embodiments, the enhancer is an M97 protein. In some embodiments, the M97 protein comprises an amino acid sequence that has at least 90%, 91%, 92%, 93%, 94%, 95%, 96%, 97%, 98%, 99%, or 100% identity to SEQ ID NO: 1500. In some embodiments, the M97 protein is encoded by a nucleotide sequence that has at least 90%, 91%, 92%, 93%, 94%, 95%, 96%, 97%, 98%, 99%, or 100% identity to SEQ ID NO: 1499. In some embodiments, the enhancer is a KSHV ORF36 protein. In some embodiments, the KSHV ORF36 protein comprises an amino acid sequence that has at least 90%, 91%, 92%, 93%, 94%, 95%, 96%, 97%, 98%, 99%, or 100% identity to SEQ ID NO: 1576. In some embodiments, the KSHV ORF36 protein is encoded by a nucleotide sequence that has at least 90%, 91%, 92%, 93%, 94%, 95%, 96%, 97%, 98%, 99%, or 100% identity to SEQ ID NO: 1575.

In some embodiments, the enhancer is a DNA repair protein. In some embodiments, the DNA repair protein is a single-stranded endonuclease that removes 5′ flaps at the 3′ ends of a nick. In some embodiments, the DNA repair protein is flap structure-specific endonuclease 1 (FEN1) or a polynucleotide encoding FEN1. In some embodiments, the FEN1 comprises an amino acid sequence that has at least 90%, 91%, 92%, 93%, 94%, 95%, 96%, 97%, 98%, 99%, or 100% identity to any one of SEQ ID NOs: 1467, 1475, 1485, 1490, 1495, and 1510-1516. In some embodiments, the FEN1 is encoded by a nucleotide sequence that has at least 90%, 91%, 92%, 93%, 94%, 95%, 96%, 97%, 98%, 99%, or 100% identity to SEQ ID NO: 1466, 1474, 1479, 1484, 1489, 1494, 1509, and 1524-1529. In some embodiments, the DNA repair protein is the MLH1 protein. In some embodiments, the MLH1 comprises an amino acid sequence that has at least an amino acid sequence that has at least 90%, 91%, 92%, 93%, 94%, 95%, 96%, 97%, 98%, 99%, or 100% identity to any one of SEQ ID NOs: 1572 or 1573. In some embodiments, the DNA repair protein comprises a heterodimerization domain, e.g., a self-assembling coiled coil heterodimerization domain, for binding to a DNA-dependent DNA polymerase having the sequence of the other portion of the heterodimerization domain. In some embodiments, the FEN1 comprises a heterodimerization domain, e.g., a self-assembling coiled coil heterodimerization domain, for binding to a DNA-dependent DNA polymerase having the sequence of the other portion of the heterodimerization domain.

In some embodiments, the enhancer comprises a dNTP synthetic enzyme. In some embodiments, the enhancer comprises a kinase. In some embodiments, the kinase comprises a nucleoside kinase, deoxynucleoside kinase, deoxynucleoside monophosphate kinase, or deoxynucleotide diphosphate.

In some embodiments, the enhancer comprises Vpx, BGLF4, M97, KSHV ORF36 FEN1, or MLH1. In some embodiments, the enhancer comprises Vpx and FEN1. In some embodiments, the enhancer comprises M97 and FEN1.

In some embodiments, the enhancer confers an increase in gene editing. In some embodiments, the enhancer, or the combination of enhancers, confers a two-fold increase in gene editing as compared to gene editing in the absence of the enhancer, or the combination of enhancers. In some embodiments, the enhancer, or the combination of enhancers, confers a five-fold increase in gene editing. In some embodiments, the enhancer, or the combination of enhancers, confers a ten-fold increase in gene editing.

### Delivery

The following section provides additional features of lipid-based delivery compositions, including lipid nanoparticles (LNPs) and lipoplexes, for the nucleic acid or nucleic acids encoding the same. In some embodiments, the nucleic acid or nucleic acids encoding the same is delivered to the cell via at least one lipid nanoparticle (LNP).

Lipid nanoparticles (LNPs) are a well-known means for delivery of nucleotide and protein cargo, and may be used for delivery of the guide RNAs (e.g., sgRNAs, dgRNAs, or crRNAs), compositions, or pharmaceutical formulations disclosed herein. In some embodiments, the LNPs deliver nucleic acid, protein, or nucleic acid together with protein. As used herein, lipid nanoparticle (LNP) refers to a particle that comprises a plurality of (i.e., more than one) lipid molecules physically associated with each other by intermolecular forces. Any LNP known to those of skill in the art to be capable of delivering nucleotides to subjects may be utilized.

In certain embodiments, an LNP has a diameter of about 50-175 nm, or a population of the LNP with an average diameter of about 50-175 nm as measured by dynamic light scattering. In preferred embodiments, an LNP composition has a diameter of 75-150 nm.

In embodiments, the average diameter is a Z-average diameter. In certain embodiments, the Z-average diameter is measured by dynamic light scattering (DLS) using methods known in the art. For example, average particle size and polydispersity can be measured by dynamic light scattering (DLS) using a Malvern Zetasizer DLS instrument. LNP samples are diluted with PBS buffer prior to being measured by DLS. Z-average diameter and number average diameter along with a polydispersity index (pdi) can be determined. The Z average is the intensity weighted mean hydrodynamic size of the ensemble collection of particles. The number average is the particle number weighted mean hydrodynamic size of the ensemble collection of particles. A Malvern Zetasizer instrument can also be used to measure the zeta potential of the LNP using methods known in the art.

In some embodiments, Dynamic Light Scattering (“DLS”) may be used to characterize the polydispersity index (PDI) and size of the LNPs of the present disclosure. DLS measures the scattering of light that results from subjecting a sample to a light source. PDI, as determined from DLS measurements, represents the distribution of particle size (around the mean particle size) in a population, with a perfectly uniform population having a PDI of zero.

In some embodiments, the LNPs disclosed herein have a PDI from about 0.005 to about 0.75. In some embodiments, the LNPs disclosed herein have a PDI from about 0.005 to about 0.1. In some embodiments, the LNPs disclosed herein have a PDI from about 0.005 to about 0.09, about 0.005 to about 0.08, about 0.005 to about 0.07, or about 0.006 to about 0.05. In some embodiments, the LNP may have a PDI from about 0.01 to about 0.5. In some embodiments, the LNP may have a PDI from about zero to about 0.4. In some embodiments, the LNP may have a PDI from about zero to about 0.35. In some embodiments, the LNP PDI may range from about zero to about 0.3. In some embodiments, the LNP may have a PDI that may range from about zero to about 0.25. In some embodiments, the LNP PDI may range from about zero to about 0.2. In some embodiments, the LNP may have a PDI from about zero to about 0.05. In some embodiments, the LNP may have a PDI from about zero to about 0.01. In some embodiments, the LNP may have a PDI less than about 0.01, 0.02, 0.05, 0.08, 0.1, 0.15, 0.2, or 0.4.

LNP size may be measured by various analytical methods known in the art. In some embodiments, LNP size may be measured using Asymmetric-Flow Field Flow Fractionation-Multi-Angle Light Scattering (AF4-MALS). In certain embodiments, LNP size may be measured by separating particles in the composition by hydrodynamic radius, followed by measuring the molecular weights, hydrodynamic radii and root mean square radii of the fractionated particles. In some embodiments, LNP size and particle concentration may be measured by nanoparticle tracking analysis (NTA, Malvern Nanosight). In certain embodiments, LNP samples are diluted appropriately and injected onto a microscope slide. A camera records the scattered light as the particles are slowly infused through field of view. After the movie is captured, the Nanoparticle Tracking Analysis processes the movie by tracking pixels and calculating a diffusion coefficient. This diffusion coefficient can be translated into the hydrodynamic radius of the particle. Such methods may also count the number of individual particles to give particle concentration. In some embodiments, LNP size, morphology, and structural characteristics may be determined by cryo-electron microscopy (“cryo-EM”).

In some embodiments, the LNPs of the LNP compositions disclosed herein can have a size (e.g. Z-average diameter or number-average diameter) of about 1 to about 250 nm. In some embodiments, the LNPs have a size of about 10 to about 200 nm. In further embodiments, the LNPs have a size of about 20 to about 150 nm. In some embodiments, the LNPs have a size of about 50 to about 150 nm or about 70 to 130 nm. In some embodiments, the LNPs have a size of about 50 to about 100 nm. In some embodiments, the LNPs have a size of about 50 to about 120 nm. In some embodiments, the LNPs have a size of about 60 to about 100 nm. In some embodiments, the LNPs have a size of about 75 to about 150 nm. In some embodiments, the LNPs have a size of about 75 to about 120 nm. In some embodiments, the LNPs have a size of about 75 to about 100 nm. In some embodiments, the LNPs have a size of about 50 to about 145 nm, about 50 to about 120 nm, about 50 to about 120 nm, about 50 to about 115 nm, about 50 to about 100 nm, about 60 to about 145 nm, about 60 to about 120 nm, about 60 to about 115 nm, or about 60 to about 100 nm. In some embodiments, the LNPs have a size of less than about 145 nm, less than about 120 nm, less than about 115 nm, less than about 100 nm, or less than about 80 nm. In some embodiments, the LNPs have a size of greater than about 50 nm or greater than about 60 nm. In some embodiments, the particle size is a Z-average particle size. In some embodiments, the particle size is a number-average particle size. In some embodiments, the particle size is the size of an individual LNP. Unless indicated otherwise, all sizes referred to herein are the average sizes (diameters) of the fully formed nanoparticles, as measured by dynamic light scattering on a Malvern Zetasizer or Wyatt NanoStar. The nanoparticle sample is diluted in phosphate buffered saline (PBS) so that the count rate is approximately 200-400 kcps.

LNPs are formed by precise mixing a lipid component (e.g., in ethanol) with an aqueous nucleic acid component and LNPs are uniform in size. Lipoplexes are particles formed by bulk mixing the lipid and nucleic acid components and are between about 100 nm and 1 micron in size. In certain embodiments the lipid nucleic acid assemblies are LNPs.

As used herein, a “lipid nucleic acid assembly” comprises a plurality of (i.e., more than one) lipid molecules physically associated with each other by intermolecular forces. A lipid nucleic acid assembly may comprise a bioavailable lipid having a pKa value of <7.5 or <7. The lipid nucleic acid assemblies are formed by mixing an aqueous nucleic acid-containing solution with an organic solvent-based lipid solution, e.g., 100% ethanol. Suitable solutions or solvents include or may contain: water, PBS, Tris buffer, NaCl, citrate buffer, ethanol, chloroform, diethylether, cyclohexane, tetrahydrofuran, methanol, isopropanol. A pharmaceutically acceptable buffer may optionally be comprised in a pharmaceutical formulation comprising the lipid nucleic acid assemblies, e.g., for an ex vivo ACT therapy. In some embodiments, the aqueous solution comprises an RNA, such as an mRNA or a template guide RNA. In some embodiments, the aqueous solution comprises an mRNA encoding an RNA-guided DNA binding agent, such as a SpyCas9 nickase. In some embodiments, the aqueous solution comprises an mRNA encoding a DNA-dependent DNA polymerase.

In some embodiments, the lipid nucleic acid assembly formulations include an “amine lipid” (sometimes herein or elsewhere described as an “ionizable lipid” or a “biodegradable lipid”), together with an optional “helper lipid”, a “neutral lipid”, and a stealth lipid such as a PEG lipid. In some embodiments, the amine lipids or ionizable lipids are cationic depending on the pH.

### Ionizable/Amine Lipids

In some embodiments, LNPs comprise an ionizable lipid such as those provided herein. In some embodiments, LNPs comprise an ionizable lipid such as Lipid A, or Lipid D or their equivalents, including acetal analogs of Lipid A or Lipid D.

In some embodiments, the ionizable lipid (e.g., amine lipid) is represented by Formula (I),

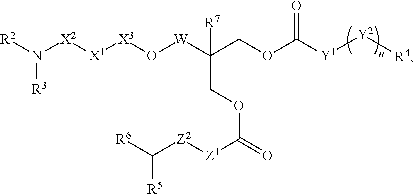

wherein
X¹ is O, NR¹, or a direct bond,
X² is C₂₋₅ alkylene,
X³ is C(═O) or a direct bond,
R¹ is H or Me,
R³ is C₁₋₃ alkyl,
R² is C₁₋₃ alkyl, or
R² taken together with the nitrogen atom to which it is attached and 1-3 carbon atoms of X² form a 4-, 5-, or 6-membered ring, or
X¹ is NR¹, R¹ and R² taken together with the nitrogen atoms to which they are attached form a 5- or 6-membered ring, or
R² taken together with R3 and the nitrogen atom to which they are attached form a 5-, 6-, or 7-membered ring,
Y¹ is C2-12 alkylene,
Y² is selected from

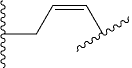 [SMILES: */C=C\C*]

(in either orientation),

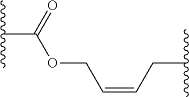 [SMILES: *C/C=C\COC(*)=O]

(in either orientation),

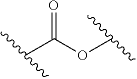 [SMILES: *OC(*)=O]

(in either orientation),
n is 0 to 3,
R⁴ is C₁₋₁₅ alkyl,
Z¹ is C₂₋₆ alkylene or a direct bond,
   Z² is

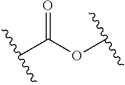 [SMILES: *OC(*)=O]

(in either orientation) or absent, provided that if Z¹ is a direct bond, Z² is absent;
R⁵ is C₅₋₉ alkyl or C₆₋₁₀ alkoxy,
R⁶ is C₅₋₉ alkyl or C₆₋₁₀ alkoxy,
W is methylene or a direct bond, and
R⁷ is H or Me,
or a salt thereof,
provided that if R³ and R² are C₂ alkyls, X¹ is O, X² is linear C₃ alkylene, X³ is
C(═O), Y¹ is linear C₆ alkylene, (Y²)n-R⁴ is

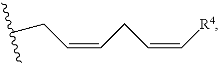

R⁴ is linear C₅ alkyl, Z¹ is C₂ alkylene, Z² is absent, W is methylene, and R⁷ is H, then R⁵ and R⁶ are not C₈ alkoxy,
or a salt thereof.

In some embodiments, the ionizable lipid (e.g., amine lipid) is Lipid A, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. Lipid A can be depicted as:

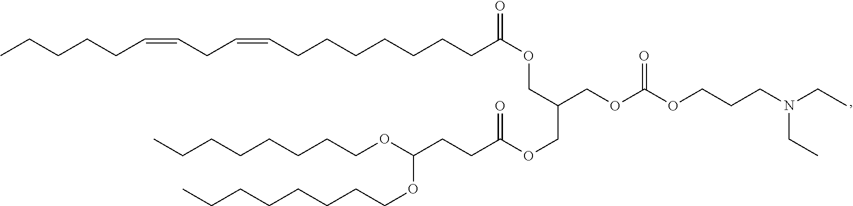 [SMILES: CCCCC/C=C\C/C=C\CCCCCCCC(=O)OCC(COC(=O)CCC(OCCCCCCCC)OCCCCCCCC)COC(=O)OCCCN(CC)CC]

or a salt thereof.

Lipid A may be synthesized according to WO2015/095340 (e.g., pp. 84-86). In some embodiments, the ionizable lipid (e.g., amine lipid) is Lipid A, or an ionizable lipid (e.g., amine lipid) provided in WO2020/219876, which is hereby incorporated by reference.

In some embodiments, the ionizable lipid (e.g., amine lipid) is represented by structural Formula (II),

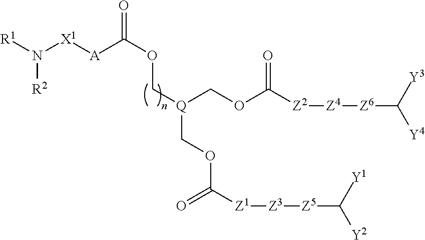

or a salt thereof,
wherein,
Q is CR³ or

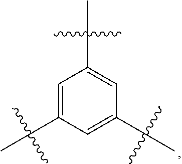 [SMILES: *c1cc(*)cc(*)c1]

A is O, NH, or a direct bond,
X¹ is a C₁₋₅ alkylene,
R¹ and R² are each independently a C₁₋₃ alkyl, or
R¹ taken together with the nitrogen atom to which it is attached and 1-3 carbon atoms of X¹ form a 4-, 5-, or 6-membered ring, or
R¹ taken together with R2 and the nitrogen atom to which they are attached form a 5-, 6-, or 7-membered ring, and
R³ is H or C₁₋₃ alkyl,
Z¹ and Z² are each independently a C1-5 alkylene,
Z³ and Z⁴ are each independently a —C(═O)O— in either direction,
Z⁵ and Z⁶ are each independently a direct bond or a C1-3 alkylene,
Y¹ is selected from H, a C₁₋₁₀ alkyl, C₃₋₁₀ alkenyl, and C₃₋₁₀ alkynyl,
Y², Y³, and Y⁴ are each independently selected from a C₃₋₁₀ alkyl, C₃₋₁₀ alkenyl, or C₃₋₁₀ alkynyl, and
n is 0 or 1,
or a salt thereof.

In some embodiments, the ionizable lipid (e.g., amine lipid) is Lipid B, which is O,O′-(2-((((2-(diethylamino)ethyl)carbamoyl)oxy)methyl)propane-1,3-diyl) di(heptadecan-9-yl) diglutarate. Lipid B can be depicted as:

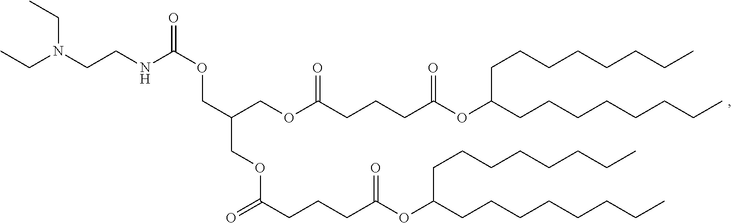 [SMILES: CCCCCCCCC(CCCCCCCC)OC(=O)CCCC(=O)OCC(COC(=O)CCCC(=O)OC(CCCCCCCC)CCCCCCCC)COC(=O)NCCN(CC)CC]

or a salt thereof.

Lipid B may be synthesized as follows: to a solution of di(heptadecan-9-yl) O,O′-(2-((((4-nitrophenoxy)carbonyl)oxy)methyl)propane-1,3-diyl) diglutarate in MeCN (0.05-0.25 M) was added N′,N′-diethylethane-1,2-diamine (1.0-3.0 equiv.), pyridine (1.0-2.0 equiv.) and DMAP (0.1-1.0 equiv.). Then the mixture was stirred at 15-25° C. for at least 12 h under N₂ atmosphere. The reaction mixture was concentrated under reduced pressure to remove solvent. The residue was diluted with EtOAc and washed 2-5× with IN NaHCO₃ and 3× with H₂O. The organic layer was dried over Na₂SO₄, filtered and the filtrate was concentrated under reduced pressure to give a residue. The residue was purified by silica gel chromatography to afford product as a colorless oil. ¹H NMR (400 MHZ, CDCl₃) δ 5.35 (s, 1H), 4.85 (p, J=6.3 Hz, 2H), 4.12 (t, J=6.0 Hz, 6H), 3.22 (q, J=5.8 Hz, 2H), 2.53 (d, J=6.8 Hz, 6H), 2.36 (dt, J=14.7, 7.4 Hz, 9H), 1.93 (p, J=7.5 Hz, 4H), 1.49 (q, J=5.9 Hz, 8H), 1.24 (s, 48H), 1.00 (t, J=7.1 Hz, 6H), 0.87 (t, J=6.7 Hz, 12H). MS: 953.6 m/z [M+H].

In some embodiments, the ionizable lipid (e.g., amine lipid) is Lipid C, which is 3-(((2-(azepan-1-yl)ethyl)carbamoyl)oxy)-2-((((9Z,12Z)-octadeca-9,12-dienoyl)oxy)methyl)propyl heptadecan-9-yl glutarate. Lipid C can be depicted as:

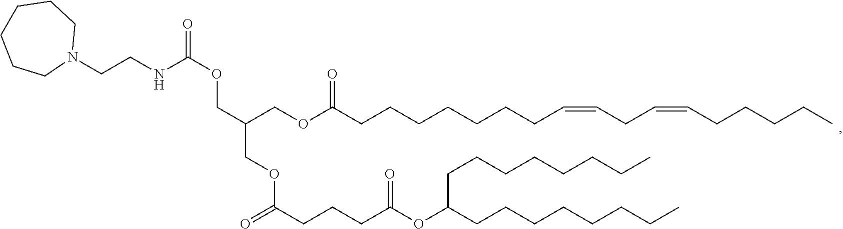 [SMILES: CCCCC/C=C\C/C=C\CCCCCCCC(=O)OCC(COC(=O)CCCC(=O)OC(CCCCCCCC)CCCCCCCC)COC(=O)NCCN1CCCCCC1]

or a salt thereof.

Lipid C may be synthesized from heptadecan-9-yl (3-(((4-nitrophenoxy)carbonyl)oxy)-2-((((9Z,12Z)-octadeca-9,12-dienoyl)oxy)methyl)propyl) glutarate and 2-(azepan-1-yl)ethan-1-amine using the same method employed for Lipid B. ¹H NMR (400 MHZ, CDCl₃) δ 5.44-5.26 (m, 5H), 4.86 (p, J=6.3 Hz, 1H), 4.13 (t, J=5.8 Hz, 6H), 3.21 (t, J=6.0 Hz, 2H), 2.81-2.73 (m, 2H), 2.66-2.56 (m, 5H), 2.42-2.26 (m, 7H), 2.05 (q, J=6.8 Hz, 4H), 1.94 (p, J=7.5 Hz, 2H), 1.62 (d, J=16.8 Hz, 10H), 1.50 (d, J=6.2 Hz, 4H), 1.41-1.26 (m, 24H), 1.25 (s, 16H), 0.88 (td, J=6.9, 5.0 Hz, 9H). MS: 890.5 m/z [M+H].

In some embodiments, the ionizable lipid is represented by structural Formula (III),

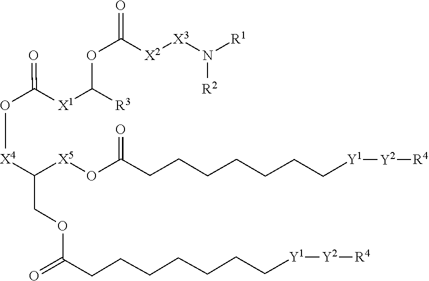

wherein, independently for each occurrence,
X¹ is C₁₋₃ alkylene or

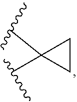 [SMILES: *C1(*)CC1]

X² is selected from O, NH, NMe, and a bond, provided that when X² is O, R² taken together with the nitrogen atom and either R¹ or a carbon atom of X³ form a 4-membered, 5-membered, or 6-membered ring;
X³ is C₂₋₄ alkylene,
X⁴ is C1 alkylene or a bond,
X⁵ is C1 alkylene or a bond,
R¹ is C₁₋₃ alkyl,
R² is C₁₋₃ alkyl, or
R² taken together with the nitrogen atom and either R¹ or a carbon atom of X³ form a 4-membered, 5-membered, or 6-membered ring,
Y¹ is selected from a bond, —CH═CH—, —(C═O)O—, and —O(C═O)—,
Y² is selected from —CH2-CH═CH—and C₃-C₄ alkylene,
R³ is selected from H, C₅₋₇ cycloalkyl, C₈-C₁₀ alkenyl, and C₃₋₁₈ alkyl, and
R⁴ is C₄₋₈ alkyl,
or a salt thereof.

In some embodiments, the ionizable lipid is Lipid D, which is 2-((4-(((3-(pyrrolidin-1-yl)propoxy)carbonyl)oxy)decanoyl)oxy)propane-1,3-diyl (9Z,9′Z,12Z,12′Z)-bis(octadeca-9,12-dienoate). Lipid D can be depicted as:

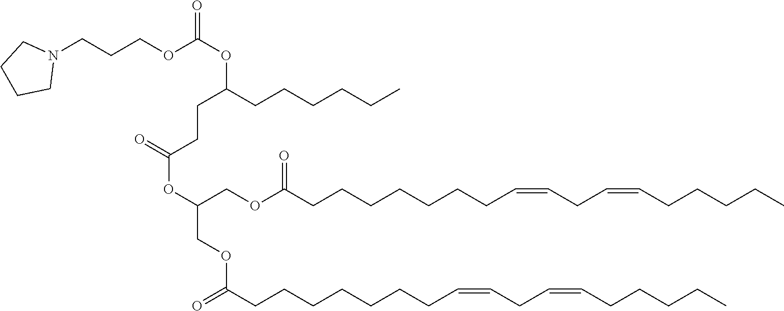 [SMILES: CCCCC/C=C\C/C=C\CCCCCCCC(=O)OCC(COC(=O)CCCCCCC/C=C\C/C=C\CCCCC)OC(=O)CCC(CCCCCC)OC(=O)OCCCN1CCCC1]

or a salt thereof.

Lipid D may be synthesized according to WO 2020/118041, which is incorporated by reference in its entirety. In some embodiments, the ionizable lipid is Lipid D, or an ionizable lipid provided in WO 2020/118041, which is hereby incorporated by reference.

In some embodiments, the ionizable lipid is represented by structural Formula (IV),

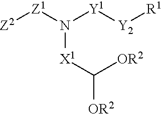

wherein, independently for each occurrence,
X¹ is C₅₋₁₁ alkylene,
Y¹ is C₃₋₁₁ alkylene,
Y² is

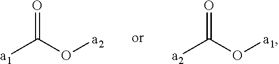

wherein a₁ is a bond to Y¹, and a₂ is a bond to R¹,
Z¹ is C₂₋₄ alkylene,
Z² is selected from —OH, —NH₂, —OC(═O)R³, —OC(═O)NHR³, —NHC(═O)NHR³, and —NHS(═O)₂R³,
R¹ is C₄₋₁₂ alkyl or C₃₋₁₂ alkenyl,
each R² is independently C₄₋₁₂ alkyl, and
R³ is C₁₋₃ alkyl,
or a salt thereof.

In some embodiments, the ionizable lipid is Lipid E, which is 8-((8,8-bis(octyloxy)octyl)(2-hydroxyethyl)amino)octyl nonanoate. Lipid E can be depicted as:

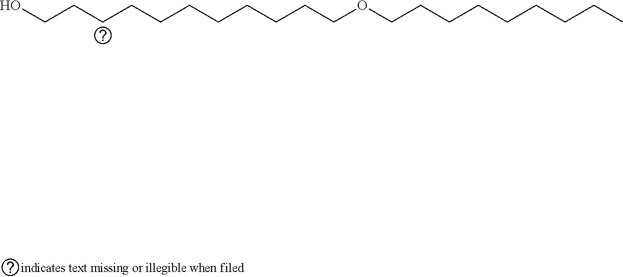 [SMILES: CCCCCCCCCOCCCCCCCCCCCO]

or a salt thereof.

In some embodiments, the ionizable lipid (e.g., amine lipid) is Lipid F, which is nonyl 8-((7,7-bis(octyloxy)heptyl)(2-hydroxyethyl)amino)octanoate. Lipid F can be depicted as:

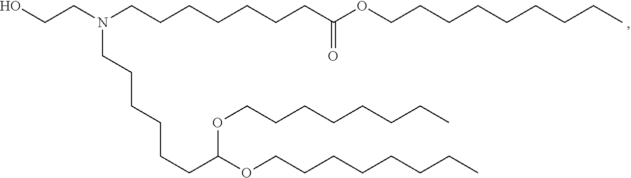 [SMILES: CCCCCCCCCOC(=O)CCCCCCCN(CCO)CCCCCCC(OCCCCCCCC)OCCCCCCCC]

or a salt thereof.

Lipids E and F may be synthesized according to WO2020072605 and Mol. Ther. 2018, 26(6), 1509-1519 (“Sabnis”), which are incorporated by reference in their entireties. In some embodiments, the ionizable lipid (e.g., amine lipid) Lipids E and F, or an ionizable lipid (e.g., amine lipid) provided in WO2020072605, which is hereby incorporated by reference.

In other embodiments, ionizable lipids can include, for example the ionizable lipids of WO 2020/219876 (e.g., at pp. 13-33, 66-87), WO 2020/118041, WO 2020/072605 (e.g., at pp. 5-12, 21-29, 61-68, WO 2019/067992, WO 2017/173054, WO 2015/095340, and WO 2014/136086, the entire contents of which, are hereby incorporated by reference.

Ionizable lipids (e.g., amine lipids) and other “biodegradable lipids” suitable for use in the lipid nucleic acid assemblies described herein are biodegradable in vivo or ex vivo. The ionizable lipids (e.g., amine lipids) have low toxicity (e.g., are tolerated in animal models without adverse effect in amounts of greater than or equal to 10 mg/kg). In some embodiments, lipid nucleic acid assemblies comprising an ionizable lipid (e.g., amine lipid) include those where at least 75% of the ionizable lipid (e.g., amine lipid) is cleared from the plasma or the engineered cell within 8, 10, 12, 24, or 48 hours, or 3, 4, 5, 6, 7, or 10 days. In some embodiments, lipid nucleic acid assemblies comprising an ionizable lipid (e.g., amine lipid) include those where at least 50% of the nucleic acid, e.g., mRNA or guide RNA, is cleared from the plasma within 8, 10, 12, 24, or 48 hours, or 3, 4, 5, 6, 7, or 10 days. In some embodiments, lipid nucleic acid assemblies comprising an ionizable lipid (e.g., amine lipid) include those where at least 50% of the lipid nucleic acid assembly is cleared from the plasma within 8, 10, 12, 24, or 48 hours, or 3, 4, 5, 6, 7, or 10 days, for example by measuring a lipid (e.g., an ionizable lipid (e.g., amine lipid)), nucleic acid, e.g., RNA/mRNA, or other component. In some embodiments, lipid-encapsulated versus free lipid, RNA, or nucleic acid component of the lipid nucleic acid assembly is measured.

Lipid clearance may be measured as described in literature. See Maier, M. A., et al. Biodegradable Lipids Enabling Rapidly Eliminated Lipid Nanoparticles for Systemic Delivery of RNAi Therapeutics. Mol. Ther. 2013, 21(8), 1570-78 (“Maier”).

Ionizable and bioavailable lipids for LNP delivery of nucleic acids known in the art are suitable. The ionizable lipids of the present disclosure may form salts depending upon the pH of the medium they are in. For example, in a slightly acidic medium, the ionizable lipids may be protonated and thus bear a positive charge. Conversely, in a slightly basic medium, such as, for example, blood where pH is approximately 7.35, the ionizable lipids may not be protonated and thus bear no charge. In some embodiments, the ionizable lipids of the present disclosure may be predominantly protonated at a pH of at least about 9. In some embodiments, the ionizable lipids of the present disclosure may be predominantly protonated at a pH of at least about 10.

The pH at which the ionizable lipids is predominantly protonated is related to its intrinsic pKa. In some embodiments, an ionizable lipid of the present disclosure has a pKa in the range of from about 5.1 to about 8.0, even more preferably from about 5.5 to about 7.6. In some embodiments, an ionizable lipid of the present disclosure has a pKa in the range of from about 5.7 to about 8, from about 5.7 to about 7.6, from about 6 to about 8, from about 6 to about 7.5, from about 6 to about 7, from about 6 to about 6.9, from about 6 to about 6.5, from about 6.1 to about 6.9, or from about 6 to about 6.85. In some embodiments, an ionizable lipid of the present disclosure has a pKa of about 6.0, about 6.1, about 6.1, about 6.2, about 6.3, about 6.4, about 6.6, about 6.7, about 6.8, or about 6.9. Alternatively, an ionizable lipid of the present disclosure has a pKa in the range of from about 6 to about 8. The pKa can be an important consideration in formulating LNPs, as it has been found that LNPs formulated with certain lipids having a pKa ranging from about 5.5 to about 7.0 are effective for delivery of cargo in vivo. Further, it has been found that LNPs formulated with certain lipids having a pKa ranging from about 5.3 to about 6.4 are effective for delivery in vivo, e.g. to tumors. See, e.g., WO 2014/136086. In some embodiments, the ionizable lipids are positively charged at an acidic pH but neutral in the blood.

Ionizable lipids (e.g., amine lipids) and other “biodegradable lipids” suitable for use in the lipid nucleic acid assemblies described herein or can be biodegradable in vivo or ex vivo. The amine lipids have low toxicity (e.g., are tolerated in animal models without adverse effect in amounts of greater than or equal to 10 mg/kg). In some embodiments, lipid nucleic acid assemblies comprising an amine lipid include those where at least 75% of the amine lipid is cleared from the plasma or the engineered cell within 8, 10, 12, 24, or 48 hours, or 3, 4, 5, 6, 7, or 10 days. In some embodiments, lipid nucleic acid assemblies comprising an amine lipid include those where at least 50% of the nucleic acid, e.g., mRNA or guide RNA, is cleared from the plasma within 8, 10, 12, 24, or 48 hours, or 3, 4, 5, 6, 7, or 10 days. In some embodiments, lipid nucleic acid assemblies comprising an amine lipid include those where at least 50% of the lipid nucleic acid assembly is cleared from the plasma within 8, 10, 12, 24, or 48 hours, or 3, 4, 5, 6, 7, or 10 days, for example by measuring a lipid (e.g., an amine lipid), nucleic acid, e.g., RNA/mRNA, or other component. In some embodiments, lipid-encapsulated versus free lipid, RNA, or nucleic acid component of the lipid nucleic acid assembly is measured.

Biodegradable lipids include, for example the biodegradable lipids of WO 2020/219876 (e.g., at pp. 13-33, 66-87), WO 2020/118041, WO 2020/072605 (e.g., at pp. 5-12, 21-29, 61-68, WO 2019/067992, WO 2017/173054, WO 2015/095340, and WO 2014/136086, and LNPs include LNP compositions described therein, the lipids and compositions of which are hereby incorporated by reference.

Lipid clearance may be measured as described in literature. See Maier, M. A., et al. Biodegradable Lipids Enabling Rapidly Eliminated Lipid Nanoparticles for Systemic Delivery of RNAi Therapeutics. Mol. Ther. 2013, 21(8), 1570-78 (“Maier”).

Ionizable and bioavailable lipids for LNP delivery of nucleic acids known in the art are suitable. Lipids may be ionizable depending upon the pH of the medium they are in. For example, in a slightly acidic medium, the lipid, such as an amine lipid, may be protonated and thus bear a positive charge. Conversely, in a slightly basic medium, such as, for example, blood where pH is approximately 7.35, the lipid, such as an amine lipid, may not be protonated and thus bear no charge.

The ability of a lipid to bear a charge is related to its intrinsic pKa. In some embodiments, the amine lipids of the present disclosure may each, independently, have a pKa in the range of about 5.1-7.4.

### Additional Lipids

“Neutral lipids” suitable for use in a lipid composition of the disclosure include, for example, a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), 1,2-dioleoyl-sn-glycero-3-phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In certain embodiments, the neutral phospholipid may be selected from distearoylphosphatidylcholine (DSPC) and dimyristoyl phosphatidyl ethanolamine (DMPE). In certain embodiments, the neutral phospholipid may be selected from the group consisting of distearoylphosphatidylcholine (DSPC) and dimyristoyl phosphatidyl ethanolamine (DMPE). In other embodiments, the neutral phospholipid may be distearoylphosphatidylcholine (DSPC).

“Helper lipids” include steroids, sterols, and alkyl resorcinols. Helper lipids suitable for use in the present disclosure include, but are not limited to, cholesterol, 5-heptadecylresorcinol, and cholesterol hemisuccinate. In certain embodiments, the helper lipid may be cholesterol or a derivative thereof. In one embodiment, the helper lipid may be cholesterol hemisuccinate.

“Stealth lipids” are lipids that alter the length of time the nanoparticles can exist in vivo (e.g., in the blood). Stealth lipids may assist in the formulation process by, for example, reducing particle aggregation and controlling particle size. Stealth lipids used herein may modulate pharmacokinetic properties of the lipid nucleic acid assembly or aid in stability of the nanoparticle ex vivo. Stealth lipids suitable for use in a lipid composition of the disclosure include, but are not limited to, stealth lipids having a hydrophilic head group linked to a lipid moiety. Stealth lipids suitable for use in a lipid composition of the present disclosure and information about the biochemistry of such lipids can be found in Romberg et al., Pharmaceutical Research, Vol. 25, No. 1, 2008, pg. 55-71 and Hoekstra et al., Biochimica et Biophysica Acta 1660 (2004) 41-52. Additional suitable PEG lipids are disclosed, e.g., in WO 2006/007712.

In one embodiment, the hydrophilic head group of stealth lipid comprises a polymer moiety selected from polymers based on PEG. Stealth lipids may comprise a lipid moiety. In some embodiments, the stealth lipid is a PEG lipid.

In one embodiment, a stealth lipid comprises a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids and poly[N-(2-hydroxypropyl)methacrylamide].

In one embodiment, the PEG lipid comprises a polymer moiety based on PEG (sometimes referred to as poly(ethylene oxide)).

The PEG lipid further comprises a lipid moiety. In some embodiments, the lipid moiety may be derived from diacylglycerol or diacylglycamide, including those comprising a dialkylglycerol or dialkylglycamide group having alkyl chain length independently comprising from about C₄ to about C40 saturated or unsaturated carbon atoms, wherein the chain may comprise one or more functional groups such as, for example, an amide or ester. In some embodiments, the alkyl chain length comprises about C10 to C20. The dialkylglycerol or dialkylglycamide group can further comprise one or more substituted alkyl groups. The chain lengths may be symmetrical or asymmetrical.

Unless otherwise indicated, the term “PEG” as used herein means any polyethylene glycol or other polyalkylene ether polymer. In one embodiment, PEG is an optionally substituted linear or branched polymer of ethylene glycol or ethylene oxide. In one embodiment, PEG is unsubstituted. In one embodiment, the PEG is substituted, e.g., by one or more alkyl, alkoxy, acyl, hydroxy, or aryl groups. In one embodiment, the term includes PEG copolymers such as PEG-polyurethane or PEG-polypropylene (see, e.g., J. Milton Harris, Poly (ethylene glycol) chemistry: biotechnical and biomedical applications (1992)); in another embodiment, the term does not include PEG copolymers. In one embodiment, the PEG has a molecular weight of from about 130 to about 50,000, and in a sub-embodiment, about 1,500 to about 2,500.

In some embodiments, the PEG (e.g., conjugated to a lipid moiety or lipid, such as a stealth lipid), is a “PEG-2K,” also termed “PEG 2000,” which has an average molecular weight of about 2,000 Daltons. PEG-2K is represented herein by the following formula (IV), wherein n is 45, meaning that the number averaged degree of polymerization comprises about 45 subunits

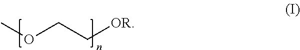

However, other PEG embodiments known in the art may be used, including, e.g., those where the number-averaged degree of polymerization comprises about 23 subunits (n=23), or 68 subunits (n=68). In some embodiments, R may be selected from H, substituted alkyl, and unsubstituted alkyl. In some embodiments, R may be unsubstituted alkyl. In some embodiments, R may be methyl.

In any of the embodiments described herein, the PEG lipid may be selected from PEG-dilauroylglycerol, PEG-dimyristoylglycerol (PEG-DMG; catalog #GM-020 from NOF, Tokyo, Japan), such as e.g., 1,2-dimyristoyl-rac-glycero-3-methylpolyoxyethylene glycol 2000 (PEG2k-DMG), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide, PEG-dipalmitoylglycamide, and PEG-distearoylglycamide, PEG-cholesterol (1-[8′-(Cholest-5-en-3 [beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMPE) (cat. #880150P from Avanti Polar Lipids, Alabaster, Alabama, USA), 1,2-distearoyl-sn-glycero-3-phosphoethanolamine-N-[methoxy(polyethylene glycol)-2000] (PEG2k-DSPE) (cat. #880120C from Avanti Polar Lipids, Alabaster, Alabama, USA), 1,2-distearoyl-sn-glycerol, methoxypolyethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy(polyethylene glycol)-2000] (PEG2k-DSA), and methoxy-PEG2000-carbamoyl-1,2-tridecyoxypropylamine) (C13 Ether), and methoxy-PEG2000-carbamoyl-1,2-tetradecyoxypropylamine (C14 Ether). In one embodiment, the PEG lipid may be 1,2-dimyristoyl-rac-glycero-3-methylpolyoxyethylene glycol 2000. In one embodiment, the PEG lipid may be PEG2k-DMG. In some embodiments, the PEG lipid may be PEG2k-DSG. In one embodiment, the PEG lipid may be PEG2k-DSPE. In one embodiment, the PEG lipid may be PEG2k-DMA. In one embodiment, the PEG lipid may be PEG2k-C-DMA. In one embodiment, the PEG lipid may be compound S027, disclosed in WO2016/010840 (paragraphs [00240] to [00244]). In one embodiment, the PEG lipid may be PEG2k-DSA. In one embodiment, the PEG lipid may be PEG2k-C11. In some embodiments, the PEG lipid may be PEG2k-C14. In some embodiments, the PEG lipid may be PEG2k-C16. In some embodiments, the PEG lipid may be PEG2k-C18.

In some embodiments, the PEG lipid includes a glycerol group. In some embodiments, the PEG lipid includes a dimyristoylglycerol (DMG) group. In some embodiments, the PEG lipid comprises PEG-2k. In some embodiments, the PEG lipid is a PEG-DMG. In some embodiments, the PEG lipid is a PEG-2k-DMG. In some embodiments, the e PEG lipid is 1,2-dimyristoyl-rac-glycero-3-methoxypolyethylene glycol2000. In some embodiments, the PEG-2k-DMG is 1,2-dimyristoyl-rac-glycero-3-methoxypolyethylene glycol-2000.

### Lipid Nanoparticles (LNPs)

The LNP may contain (i) an ionizable (biodegradable) lipid (e.g., amine lipid), (ii) an optional neutral lipid, (iii) a helper lipid, and (iv) a stealth lipid, such as a PEG lipid. The lipid nucleic acid assembly may contain a biodegradable lipid and one or more of a neutral lipid, a helper lipid, and a stealth lipid, such as a PEG lipid.

The lipid nucleic acid assembly may contain (i) an ionizable lipid (e.g., amine lipid) for encapsulation and for endosomal escape, (ii) a neutral lipid for stabilization, (iii) a helper lipid, also for stabilization, and (iv) a stealth lipid, such as a PEG lipid. The lipid nucleic acid assembly may contain an ionizable lipid (e.g., amine lipid) and one or more of a neutral lipid, a helper lipid, also for stabilization, and a stealth lipid, such as a PEG lipid. (e.g., amine lipid)

An LNP may comprise a nucleic acid, e.g., an RNA, component that includes one or more of a Spy Cas9 nickase a DNA-dependent DNA polymerase, an enhancer of template-based editing, and a template guide RNA as the RNA component. In some embodiments, an LNP may comprise the RNA component, an ionizable lipid (e.g., amine lipid), a helper lipid, a neutral lipid, and a stealth lipid. In certain LNPs, the helper lipid is cholesterol. In other compositions, the neutral lipid is DSPC. In additional embodiments, the stealth lipid is PEG2k-DMG or PEG2k-C11. In some embodiments, the LNP comprises Lipid A-F or an equivalent of Lipid A-F; a helper lipid; a neutral lipid; a stealth lipid; and an RNA such as a guide RNA. In some embodiments, the LNP comprises Lipid A-F or an equivalent of Lipid A-F; a helper lipid; a stealth lipid; and an RNA such as a guide RNA. In some compositions, the ionizable lipid (e.g., amine lipid) is Lipid A-F. In some compositions, the ionizable lipid (e.g., amine lipid) is Lipid A-F or an acetal analog thereof; the helper lipid is cholesterol; the neutral lipid is DSPC; and the stealth lipid is PEG2k-DMG. In some compositions, the amine lipid is Lipid A-F or an acetal analog thereof; the helper lipid is cholesterol; the neutral lipid is DSPC; and the stealth lipid is C13 Ether.

In some embodiments, lipid nanoparticle (LNP) compositions are described according to the respective molar ratios of the component lipids in the formulation. Embodiments of the present disclosure provide lipid nanoparticle (LNP) compositions described according to the respective molar ratios of the component lipids in the formulation. In one embodiment, the mol % of the amine lipid may be from about 30 mol % to about 60 mol %.

Embodiments of the present disclosure provide LNPs described according to the respective molar ratios of the component lipids in the composition. All mol % numbers are given as a fraction of the lipid component of the lipid composition or, more specifically, the LNP compositions. In some embodiments, the lipid mol % of a lipid relative to the lipid component will be ±30%, ±25%, ±20%, ±15%, ±10%, ±5%, or ±2.5% of the specified, nominal, or actual mol % of the lipid. In some embodiments, the lipid mol % of a lipid relative to the lipid component will be ±4 mol %, ±3 mol %, ±2 mol %, ±1.5 mol %, ±1 mol %, ±0.5 mol %, ±0.25 mol %, or ±0.05 mol % of the specified, nominal, or actual mol % of the lipid component. In certain embodiments, the lipid mol % will vary by less than 15%, 10%, 5%, 1%, or 0.5% from the specified, nominal, or actual mol % of the lipid. In some embodiments, the mol % numbers are based on nominal concentration. As used herein, “nominal concentration” refers to concentration based on the input amounts of substances combined to form a resulting composition. For example, if 100 mg of solute is added to 1 L water, the nominal concentration is 100 mg/L. In some embodiments, the mol % numbers are based on actual concentration, e.g., concentration determined by an analytic method. In some embodiments, actual concentration of the lipids of the lipid component may be determined, for example, from chromatography, such as liquid chromatography, followed by a detection method, such as charged aerosol detection. In some embodiments, actual concentration of the lipids of the lipid component may be characterized by lipid analysis, AF4-MALS, NTA, and/or cryo-EM. All mol % numbers are given as a percentage of the lipids of the lipid component.

In one embodiment, the mol % of the ionizable lipid may be from about 30 mol % to about 60 mol %. In one embodiment, the mol % of the neutral lipid may be from about 5 mol % to about 15 mol %.

In one embodiment, the mol % of the helper lipid may be from about 20 mol % to about 60 mol %. In one embodiment, the mol % of the helper lipid is adjusted based on the ionizable lipid (e.g., amine lipid), neutral lipid, and PEG lipid concentrations to bring the lipid component to 100 mol %. In one embodiment, the mol % of the PEG lipid may be from about 1 mol % to about 10 mol %.

Embodiments of the present disclosure provide LNP compositions, for example, LNP compositions comprising an ionizable lipid (e.g., amine lipid) (e.g., Lipid A-F or an analog thereof), a helper lipid, a helper lipid, and a PEG lipid, described according to the respective molar ratios of the component lipids in the formulation. In certain embodiments, the amount of the ionizable lipid is from about 25 mol % to about 45 mol %; the amount of the neutral lipid is from about 10 mol % to about 30 mol %; the amount of the helper lipid is from about 25 mol % to about 65 mol %; and the amount of the PEG lipid is from about 1.5 mol % to about 3.5 mol %.

Other embodiments of the present disclosure provide LNP compositions, for example, LNP compositions comprising an ionizable lipid (e.g., Lipid A-F or an analog thereof), a helper lipid, a helper lipid, and a PEG lipid, described according to the respective molar ratios of the component lipids in the formulation. In certain embodiments, the amount of the ionizable lipid is from about 25 mol % to about 50 mol %; the amount of the neutral lipid is from about 7 mol % to about 25 mol %; the amount of the helper lipid is from about 39 mol % to about 65 mol %; and the amount of the PEG lipid is from about 0.5 mol % to about 1.8 mol %.

In certain embodiments, the amount of the ionizable lipid (e.g., amine lipid) (e.g., Lipid A-F or an analog thereof) is about 20-55 mol %. In certain embodiments, the amount of the neutral lipid is about 7-25 mol %. In certain embodiments, the amount of the helper lipid is about 39-65 mol %. In certain embodiments, the amount of the PEG lipid is about 0.5-1.8 mol %.

In some embodiments, the cargo includes an mRNA encoding an RNA-guided DNA-binding agent (e.g., a Cas nuclease, a Class 2 Cas nuclease, or Cas9), an mRNA encoding a DNA-dependent DNA polymerase, or a template guide RNA or a nucleic acid encoding a template guide RNA, or a combination of mRNA and template guide RNA. In one embodiment, an LNP comprising this cargo may contain an ionizable lipid, e.g., an ionizable lipid provided herein. In some embodiments, an LNP comprising this cargo may comprise Lipid A. In some embodiments, an LNP comprising this cargo may comprise Lipid B. In some embodiments, an LNP comprising this cargo may comprise Lipid C. In some embodiments, an LNP comprising this cargo may comprise Lipid D. In some embodiments, an LNP comprising this cargo may comprise Lipid E. In some embodiments, an LNP comprising this cargo may comprise Lipid F.

In one embodiment, an LNP may comprise a Lipid A or its equivalents, or an amine lipid as provided in WO2020219876; or Lipid D or Lipid F or an amine lipid provided in WO2020/072605. In some aspects, the amine lipid is Lipid A, or Lipid D or Lipid F. In some aspects, the amine lipid is a Lipid A equivalent, e.g. an analog of Lipid A, or an amine lipid provided in WO2020/219876. In certain aspects, the amine lipid is an acetal analog of Lipid A, optionally, an amine lipid provided in WO2020/219876. In some aspects, the amine lipid is a Lipid D or Lipid For an amine lipid found in in W2020072605. In various embodiments, a LNP comprises an amine lipid, a neutral lipid, a helper lipid, and a PEG lipid. In some embodiments, the helper lipid is cholesterol. In some embodiments, the neutral lipid is DSPC. In specific embodiments, PEG lipid is PEG2k-DMG. In some embodiments, a LNP may comprise an amine lipid (e.g., Lipid A, Lipid B, Lipid C, Lipid D, Lipid E, or Lipid F), a helper lipid, a neutral lipid, and a PEG lipid. In some embodiments, a LNP comprises an amine lipid (e.g., Lipid A, Lipid B, Lipid C, Lipid D, Lipid E, or Lipid F), DSPC, cholesterol, and a PEG lipid. In some embodiments, the LNP comprises a PEG lipid comprising DMG. In some embodiments, the amine lipid is selected from Lipid A, and an equivalent of Lipid A, including an acetal analog of Lipid A, or an amine lipid provided in WO2020/219876; or Lipid D; or Lipid F or an amine lipid provided in WO2020/072605. In additional embodiments, a LNP comprises Lipid A, cholesterol, DSPC, and PEG2k-DMG. In additional embodiments, a LNP comprises Lipid B, cholesterol, DSPC, and PEG2k-DMG. In additional embodiments, a LNP comprises Lipid C, cholesterol, DSPC, and PEG2k-DMG. In additional embodiments, a LNP comprises Lipid D, cholesterol, DSPC, and PEG2k-DMG. In additional embodiments, a LNP comprises Lipid E, cholesterol, DSPC, and PEG2k-DMG. In additional embodiments, a LNP comprises Lipid F, cholesterol, DSPC, and PEG2k-DMG.

Embodiments of the present disclosure also provide lipid compositions described according to the molar ratio between the positively charged amine groups of the ionizable lipid (e.g., amine lipid) (N) and the negatively charged phosphate groups (P) of the nucleic acid to be encapsulated. This may be mathematically represented by the equation N/P. In some embodiments, a LNP may comprise a lipid component that comprises an ionizable lipid (e.g., amine lipid), a helper lipid, a neutral lipid, and a helper lipid; and a nucleic acid component, wherein the N/P ratio is about 3 to 10. For example, the N/P ratio may be about 4-7, 5-7, or 6 to 7. In some embodiments, the N/P ratio may about 6, e.g., 6±1, or 6±0.5. In some embodiments, the N/P ratio may about 7, e.g., 7±1, or 7±0.5.

In some embodiments, the lipid nucleic acid assembly comprises an RNA component, which may comprise an mRNA, such as an mRNA encoding a SpyCas9 nickase or DNA-dependent DNA polymerase. In one embodiment, the RNA component may comprise a SpyCas9 nickase mRNA or a DNA-dependent DNA polymerase mRNA. In some compositions comprising an mRNA encoding a Cas nickase or a DNA-dependent DNA polymerase, the lipid nucleic acid assembly further comprises a template guide RNA nucleic acid, such as an sgRNA operably linked to a 3′ extension. In some embodiments, the RNA component comprises a SpyCas9 nickase mRNA, DNA-dependent DNA polymerase mRNA, and a template guide RNA.

In some embodiments, a LNP may comprise an mRNA encoding a SpyCas9 nickase, an ionizable lipid (e.g., amine lipid), a DNA-dependent DNA polymerase, a helper lipid, a neutral lipid, and a PEG lipid. In certain LNPs comprising an mRNA encoding a SpyCas9 nickase, or an mRNA encoding a DNA-dependent DNA polymerase, the helper lipid is cholesterol. In other compositions comprising an mRNA encoding a SpyCas9 nickase, or an mRNA encoding a DNA-dependent DNA polymerase, the neutral lipid is DSPC. In additional embodiments comprising an mRNA encoding SpyCas9 nickase, or an mRNA encoding a DNA-dependent DNA polymerase, the PEG lipid is PEG2k-DMG or PEG2k-C₁₁. In specific compositions comprising an mRNA encoding a SpyCas9 nickase, or an mRNA encoding a DNA-dependent DNA polymerase, the ionizable lipid (e.g., amine lipid) is selected from Lipid A-F and its equivalents, such as an acetal analog of Lipid A-F, or amine lipids provided in WO2020/219876; or Lipid D or Lipid F or amine lipids provided in WO2020/072605.

In some embodiments, an LNP may comprise a template guide RNA. In some embodiments, a LNP may comprise an ionizable lipid (e.g., amine lipid), a template guide RNA, a helper lipid, a neutral lipid, and a PEG lipid. In certain LNPs comprising a template guide RNA, the helper lipid is cholesterol. In some compositions comprising a template guide RNA, the neutral lipid is DSPC. In additional embodiments comprising a template guide RNA, the PEG lipid is PEG2k-DMG or PEG2k-C11. In some embodiments, the ionizable lipid (e.g., amine lipid) is selected from Lipid A, Lipid B, Lipid C, Lipid D, Lipid E, or Lipid F and its equivalents, such as an acetal analog of Lipid A-F, or amine lipids provided in WO2020/219876 and their equivalents; or Lipid D or Lipid F or amine lipids provided in WO2020/072605 and their equivalents.

In one embodiment, a LNP may comprise an sgRNA. In one embodiment, a LNP may comprise a SpyCas9 sgRNA. In some compositions comprising an sgRNA, the lipid nucleic acid assembly includes an ionizable lipid (e.g., amine lipid), a helper lipid, a neutral lipid, and a PEG lipid. In certain compositions comprising an sgRNA, the helper lipid is cholesterol. In other compositions comprising an sgRNA, the neutral lipid is DSPC. In additional embodiments comprising an sgRNA, the PEG lipid is PEG2k-DMG or PEG2k-C11. In some embodiments, the ionizable lipid (e.g., amine lipid) is selected from Lipid A, Lipid B, Lipid C, Lipid D, Lipid E, or Lipid F and its equivalents, such as acetal analogs of Lipid A, Lipid B, Lipid C, Lipid D, Lipid E, or Lipid F, or amine lipids provided in WO2020/219876; or Lipid D or Lipid F or amine lipids provided in WO2020/072605.

In some embodiments, a LNP comprises an mRNA encoding a SpyCas9 nickase, an mRNA encoding a DNA-dependent DNA polymerase, or a template guide RNA, which may be an sgRNA operably linked to a 3′ extension. In some embodiments, the RNA component further comprises an enhancer of template-based editing. In one embodiment, a LNP may comprise an ionizable lipid (e.g., amine lipid), an mRNA encoding a SpyCas9 nickase, an mRNA encoding a DNA-dependent DNA polymerase, a template guide RNA, a helper lipid, a neutral lipid, and a PEG lipid. In certain compositions comprising an mRNA encoding a SpyCas9 nickase, an mRNA encoding a DNA-dependent DNA polymerase, and a template guide RNA, the helper lipid is cholesterol. In some compositions comprising an mRNA encoding a SpyCas9 nickase, a DNA-dependent DNA polymerase, and a template guide RNA, the neutral lipid is DSPC. In additional embodiments comprising an mRNA encoding a SpyCas9 nickase, an mRNA encoding a DNA-dependent DNA polymerase, and a template guide RNA, the PEG lipid is PEG2k-DMG or PEG2k-C11. In some embodiments, the ionizable lipid (e.g., amine lipid) is selected from Lipid A, Lipid B, Lipid C, Lipid D, Lipid E, or Lipid F and its equivalents, such as acetal analogs of Lipid A-F, or amine lipids provided in WO2020/219876; or Lipid D or Lipid F and amine lipids provided in WO2020/072605.

In some embodiments, the LNPs include a SpyCas9 nickase mRNA, a DNA-dependent DNA polymerase mRNA, and at least one template guide RNA. In some embodiments, the LNP includes a ratio of template guide RNA to Spy Cas9 nickase mRNA from about 25:1-1:25 wt/wt.

In some embodiments, the LNPs include a SpyCas9 nickase mRNA, a DNA-dependent DNA polymerase mRNA, and at least one template guide RNA. In some embodiments, the LNP includes a ratio of template guide RNA to Spy Cas9 nickase mRNA from about 25:1-1:25 wt/wt.

In some embodiments, LNP compositions are formed by mixing an aqueous RNA solution with an organic solvent-based lipid solution. Suitable solutions or solvents include or may contain: water, PBS, Tris buffer, NaCl, citrate buffer, acetate buffer, ethanol, chloroform, diethylether, cyclohexane, tetrahydrofuran, methanol, isopropanol. For example, the organic solvent may be 100% ethanol. A pharmaceutically acceptable buffer, e.g., for in vivo administration of LNP compositions, may be used. In certain embodiments, a buffer is used to maintain the pH of the composition comprising LNPs at or above pH 6.5. In certain embodiments, a buffer is used to maintain the pH of the composition comprising LNPs at or above pH 7.0. In certain embodiments, the composition has a pH ranging from about 7.2 to about 7.7. In additional embodiments, the composition has a pH ranging from about 7.3 to about 7.7 or ranging from about 7.4 to about 7.6. In further embodiments, the composition has a pH of about 7.2, 7.3, 7.4, 7.5, 7.6, or 7.7. The pH of a composition may be measured with a micro pH probe. In certain embodiments, a cryoprotectant is included in the composition. Non-limiting examples of cryoprotectants include sucrose, trehalose, glycerol, DMSO, and ethylene glycol. Exemplary compositions may include up to 10% cryoprotectant, such as, for example, sucrose. In certain embodiments, the composition may comprise tris saline sucrose (TSS). In certain embodiments, the LNP composition may include about 1, 2, 3, 4, 5, 6, 7, 8, 9, or 10% cryoprotectant. In certain embodiments, the LNP composition may include about 1, 2, 3, 4, 5, 6, 7, 8, 9, or 10% sucrose. In some embodiments, the LNP composition may include a buffer. In some embodiments, the buffer may comprise a phosphate buffer (PBS), a Tris buffer, a citrate buffer, and mixtures thereof. In certain exemplary embodiments, the buffer comprises NaCl. In certain embodiments, the buffer lacks NaCl. Exemplary amounts of NaCl may range from about 20 mM to about 45 mM. Exemplary amounts of NaCl may range from about 40 mM to about 50 mM. In some embodiments, the amount of NaCl is about 45 mM. In some embodiments, the buffer is a Tris buffer. Exemplary amounts of Tris may range from about 20 mM to about 60 mM. Exemplary amounts of Tris may range from about 40 mM to about 60 mM. In some embodiments, the amount of Tris is about 50 mM. In some embodiments, the buffer comprises NaCl and Tris. Certain exemplary embodiments of the LNP compositions contain 5% sucrose and 45 mM NaCl in Tris buffer. In other exemplary embodiments, compositions contain sucrose in an amount of about 5% w/v, about 45 mM NaCl, and about 50 mM Tris at pH 7.5. The salt, buffer, and cryoprotectant amounts may be varied such that the osmolality of the overall composition is maintained. For example, the final osmolality may be maintained at less than 450 mOsm/L. In further embodiments, the osmolality is between 350 and 250 mOsm/L. Certain embodiments have a final osmolality of 300±20 mOsm/L or 310±40 mOsm/L.

In some embodiments, microfluidic mixing, T-mixing, or cross-mixing of the aqueous RNA solution and the lipid solution in an organic solvent is used. In certain aspects, flow rates, junction size, junction geometry, junction shape, tube diameter, solutions, and/or RNA and lipid concentrations may be varied. LNPs or LNP compositions may be concentrated or purified, e.g., via dialysis, centrifugal filter, tangential flow filtration, or chromatography. The LNP compositions may be stored as a suspension, an emulsion, or a lyophilized powder, for example. In some embodiments, an LNP composition is stored at 2-8° C., in certain aspects, the LNP compositions are stored at room temperature. In additional embodiments, an LNP composition is stored frozen, for example at −20° C. or −80° C. In other embodiments, an LNP composition is stored at a temperature ranging from about 0° C. to about −80° C. Frozen LNP compositions may be thawed before use, for example on ice, at room temperature, or at 25° C.

In some embodiments, lipid nucleic acid assemblies are formed by mixing an aqueous RNA solution with an organic solvent-based lipid solution, e.g., 100% ethanol. Suitable solutions or solvents include or may contain: water, PBS, Tris buffer, NaCl, citrate buffer, ethanol, chloroform, diethylether, cyclohexane, tetrahydrofuran, methanol, isopropanol. A pharmaceutically acceptable buffer, e.g., for in vivo administration of lipid nucleic acid assemblies, may be used. In some embodiments, a buffer is used to maintain the pH of the composition comprising lipid nucleic acid assemblies at or above pH 6.5. In some embodiments, a buffer is used to maintain the pH of the composition comprising lipid nucleic acid assemblies at or above pH 7.0. In some embodiments, the composition has a pH ranging from about 7.2 to about 7.7. The pH of a composition may be measured with a micro pH probe. In some embodiments, a cryoprotectant is included in the composition. Non-limiting examples of cryoprotectants include sucrose, trehalose, glycerol, DMSO, and ethylene glycol. Exemplary compositions may include up to 10% cryoprotectant, such as, for example, sucrose. In some embodiments, the LNP may include a buffer. In some embodiments, the buffer may comprise a phosphate buffer (PBS), a Tris buffer, a citrate buffer, and mixtures thereof. In some exemplary embodiments, the buffer comprises NaCl. In some embodiments, NaCl is omitted. Exemplary amounts of NaCl may range from about 20 mM to about 50 mM. In some embodiments, the buffer is a Tris buffer. Exemplary amounts of Tris may range from about 20 mM to about 60 mM. In some embodiments, the buffer comprises NaCl and Tris. Certain exemplary embodiments of the LNPs contain 5% sucrose and 45 mM NaCl in Tris buffer. In other exemplary embodiments, compositions contain sucrose in an amount of about 5% w/v, about 45 mM NaCl, and about 50 mM Tris at pH 7.5. The salt, buffer, and cryoprotectant amounts may be varied such that the osmolality of the overall formulation is maintained. For example, the final osmolality may be maintained at less than 450 mOsm/L. In further embodiments, the osmolality is between 350 and 250 mOsm/L. Certain embodiments have a final osmolality of 300+/−20 mOsm/L.

In some embodiments, microfluidic mixing, T-mixing, or cross-mixing is used. In certain aspects, flow rates, junction size, junction geometry, junction shape, tube diameter, solutions, or RNA and lipid concentrations may be varied. Lipid nucleic acid assemblies or LNPs may be concentrated or purified, e.g., via dialysis, tangential flow filtration, or chromatography. The lipid nucleic acid assemblies may be stored as a suspension, an emulsion, or a lyophilized powder, for example. In some embodiments, a LNP is stored at 2-8° C., in certain aspects, the LNPs are stored at room temperature. In additional embodiments, a LNP is stored frozen, for example at −20° C. or −80° C. In other embodiments, a LNP is stored at a temperature ranging from about 0° C. to about −80° C. Frozen LNPs may be thawed before use, for example on ice, at 4° C., at room temperature, or at 25° C. Frozen LNPs may be maintained at various temperatures, for example on ice, at 4° C., at room temperature, at 25° C., or at 37° C.

Exemplary lipid compositions, such as LNP compositions, in some embodiments, are biodegradable, for example, in that they do not accumulate to cytotoxic levels in vivo at a therapeutically effective dose. In some embodiments, the compositions do not cause an innate immune response that leads to substantial adverse effects at a therapeutic dose level. In some embodiments, the compositions provided herein do not cause toxicity at a therapeutic dose level.

In some embodiments, the concentration of the LNPs in the LNP composition is about 1-10 μg/mL, 2-10 μg/mL, 2.5-10 μg/mL, 1-5 μg/mL, 2-5 μg/mL, 2.5-5 μg/mL, 0.04 μg/mL, 0.08 μg/mL, 0.16 μg/mL, 0.25 μg/mL, 0.63 μg/mL, 1.25 μg/mL, 2.5 μg/mL, or 5 μg/mL.

### LNP Delivery

In some embodiments, provided herein is a method for delivering any one of the guide RNAs (e.g., sgRNAs, dgRNAs, crRNAs, tgRNAs with or without separable 3′ extensions) disclosed herein to a subject, wherein the guide RNA is associated with an LNP. The guide RNA/LNP is also associated with an mRNA encoding a Cas9 nickase and a DNA dependent DNA polymerase, optionally further in combination with an enhancer of template-based editing. The components may be formulated individually or in various combinations for delivery.

In some embodiments, provided herein is a composition comprising any one of the template guide RNAs disclosed and an LNP. In some embodiments, the composition further comprises an mRNA encoding Cas9-DNA-dependent DNA polymerase fusion protein. In some embodiments, the composition further comprises an mRNA encoding a first polypeptide comprising the SpyCas9 nickase and a first split intein and a second mRNA encoding a second polypeptide comprising the DNA-dependent DNA polymerase, or a portion thereof, and a second split intein. The first split-intein and the second split-intein may undergo trans-splicing reaction, resulting in the formation of a single fusion polypeptide comprising the DNA-dependent DNA polymerase and the nCas9.

In some embodiments, the composition further comprises an mRNA encoding a first polypeptide comprising the SpyCas9 nickase and a first split intein and a second mRNA encoding a second polypeptide comprising the DNA-dependent DNA polymerase, or a portion thereof, and a second split intein. The first split-intein and the second split-intein may undergo trans-splicing reaction, resulting in the formation of a single fusion polypeptide comprising the DNA-dependent DNA polymerase and the nCas9.

In some embodiments, provided herein is a method for delivering any of the guide RNAs described herein to a cell or a population of cells or a subject, including to a cell or population of cells in a subject in vivo, wherein any one or more of the components is associated with an LNP, e.g., an mRNA encoding a Cas9 nickase, an mRNA encoding a DNA-dependent DNA polymerase, and a guide RNA, e.g. a template guide RNA.

In some embodiments, a component associated with an LNP is an mRNA encoding an enhancer of template-based editing. In some embodiments, the amount of the mRNA encoding an enhancer of template-based editing is from about 2 mol % to about 20 mol % of the total RNA amount in the composition. As used herein, the total RNA amount in the composition includes the amount of the mRNA components and the amount of the template guide RNA component, if present. In some embodiments, the amount of the mRNA encoding an enhancer of template-based editing is from about 2 mol % to about 20 mol %, from about 4 mol % to about 20 mol %, from about 5 mol % to about 20 mol %, from about 8 mol % to about 20 mol %, from about 10 mol % to about 20 mol %, from about 2 mol % to about 15 mol %, from about 2 mol % to about 12 mol %, from about 5 mol % to about 15 mol %, from about 8 mol % to about 15 mol %, from about 8 mol % to about 14 mol %, from about 8 mol % to about 12 mol %, of the total RNA amount. In some embodiments, the amount of the mRNA encoding an enhancer of template-based editing is about 2, 3, 4, 5, 6, 7, 8, 9, 10, 11, 12, 13, 14, 15, 16, 17, 18, 19, or 20 mol % of the total RNA amount. In some embodiments, the amount of the mRNA encoding an enhancer of template-based editing is about 5, 6, 7, 8, 9, 10, 11, 12, 13, 14, or 15 mol % o of the total RNA amount. In some embodiments, the amount of the mRNA encoding an enhancer of template-based editing is about 8, 9, 10, 11, 12, 13, or 14 mol % of the total RNA amount.

In some embodiments, each component is formulated individually in an LNP. In some embodiments, each mRNA is formulated in a separate LNP. In some embodiments, the Cas9 nickase and the DNA-dependent DNA polymerase are encoded as a fusion protein by a single mRNA. In some embodiments, each enhancer of template-based editing is associated with a separate LNP. In certain embodiments, each mRNA is associated with a separate LNP. In certain embodiments, more than one mRNA is associated with a single LNP.

In some embodiments, the one or more LNPs include the tgRNA to one or more mRNA encoding the polypeptide or fusion protein disclosed herein from with a molar ratio of about 25:1 to about 1:25 wt/wt. In some embodiments, the LNP includes a ratio of the tgRNA to the one or more mRNA, from about 10:1 to about 1:10. As measured herein, the ratios are by weight. In some embodiments, the LNP includes a ratio of the tgRNA to the one or more mRNA. In some embodiments, ratio range is from about 8:1 to about 1:8, from about 5:1 to about 1:5, from about 3:1 to about 1:3, about 2:1 to about 1:2, about 5:1 to about 1:2, about 5:1 to about 1:1, about 3:1 to about 1:2, about 3:1 to about 1:1, about 3:1, about 2:1 to about 1:1. In some embodiments, the gRNA to mRNA ratio is about 3:1 or about 2:1. In some embodiments the ratio of gRNA to the one or more mRNA, is about 25:1, 10:1, 5:1, 3:1, 2:1, 1:1, 1:2, 1:3, 1:5, 1:10, or 1:25.

In some embodiments, the one or more LNPs include the mRNA encoding the first polypeptide comprising the SpyCas9 nickase, or a portion thereof, and the second mRNA encoding the second polypeptide comprising the DNA-dependent DNA polymerase, or a portion thereof, disclosed herein, with the molar ratio of about 1:1, 2:1, 3:1, 4:1, 5:1, 6:1, 7:1, 8:1, 9:1, 10:1, 11:1, 12:1, 13:1, 14:1, or 15:1.

In some embodiments, the one or more LNPs include the tgRNA, the mRNA encoding the first polypeptide comprising the SpyCas9 nickase and the first split intein, and the second mRNA encoding the second polypeptide comprising the DNA-dependent DNA polymerase, or a portion thereof, and the second split intein, disclosed herein, with the molar ratio of about 5:1:1, 6:1:1, 7:1:1, 8:1,1, 4:1:1, 3:1:1, 2:1:1, 1 5:2:1, 6:2:1, 7:2:1, 8:2,1, 4:2:1, 3:2:1, 2:2:1, 5:3:1, 6:3:1, 7:3:1, 8:3,1, 4:3:1, 3:3:1, 5:4:1, 6:4:1, 7:4:1, 8:4,1, 4:4:1, or 5:5:1.

In certain embodiments, each guide RNA is associated with a separate LNP, e.g., template guide associated with one LNP and nicking guide associated with one LNP. In certain embodiments, the template guide with a separate 3′ extension are formulated together with a single LNP. In certain embodiments, the template guide spacer and scaffold component is formulated separately with individual LNPs. In certain embodiments, a guide RNA or guide RNA component in combination with an mRNA is associated with one LNP. Compositions and methods for association and formulation of mRNAs and guide RNAs with LNP are known in the art and provided herein.

In some embodiments, the LNP is pretreated with a serum factor before contacting the cell. In some embodiments, the LNP is pretreated with a primate serum factor before contacting the cell. In some embodiments, the LNP is pretreated with a human serum factor before contacting the cell.

In some embodiments, the methods disclosed herein comprise preincubating a serum factor and the LNP for about 30 seconds to overnight. In some embodiments, the preincubation step comprises preincubating a serum factor and the LNP for about 1 minute to 1 hour. In some embodiments, it comprises preincubating for about 1-30 minutes.

In some embodiments, the LNP compositions are administered sequentially. In some embodiments, the LNP compositions are administered simultaneously. In some embodiments, the population of cells is contacted with 3 LNP compositions. In some embodiments, the population of cells is contacted with 4 LNP compositions.

In some embodiments, the cells are frozen between sequential contacting or editing steps.

In some embodiments, the LNP is pretreated with a serum factor before contacting the cell. In some embodiments, the LNP is pretreated with a human serum before contacting the cell. In some embodiments, the LNP is pretreated with a serum replacement, e.g., a commercially available serum replacement, preferably wherein the serum replacement is appropriate for ex vivo use.

In some embodiments, a LNP is provided to a “non-activated” cell. A “non-activated” cell refers to a cell that has not been stimulated in vitro. In some embodiments, a “non-activated” T cell may have been stimulated in vivo (e.g., by antigen) while in the body, however said cell may be referred to as non-activated herein if said cell has not been stimulated in vitro in culture. An “activated” cell is also useful in the methods disclosed herein and can refer to a cell that has been stimulated in vitro. Agents for activating cells in vitro are provided herein and are known in the art, particularly for activation of T cells or B cells.

In some embodiments, the T cell is activated prior to contact with a LNP, is activated in between contact with LNPs, or is activated after contact with a LNP.

In some embodiments, the methods disclosed herein comprise electroporation or lipofection. Electroporation is a well-known means for delivery of cargo, and any electroporation methodology may be used for delivery of any one of the guide RNAs disclosed herein. In some embodiments, electroporation may be used to deliver any one of the guide RNAs disclosed herein and SpyCas9 or a polynucleotide (e.g., mRNA or DNA) encoding SpyCas9.

### Exemplary DNA Molecules, Vectors, Expression Constructs, Host Cells, and Production Methods

In certain embodiments, the disclosure provides one or more DNA molecule comprising a sequence encoding any of the nucleic acids (e.g., mRNAs) encoding a SpyCas9 nickase, a polymerase, or a fusion protein described herein. In some embodiments, the DNA molecule may further include, but is not limited to, promoters, enhancers, and regulatory sequences.

In certain embodiments, the disclosure provides one or more DNA molecule comprising a sequence encoding one or more guide RNA as described herein. In some embodiments, the DNA molecule further may further include, but is not limited to, promoters, enhancers, and regulatory sequences. In some embodiments, the DNA molecule further comprises a promoter operably linked to the sequence encoding a guide RNA described herein.

In some embodiments, the DNA molecule further comprises a promoter operably linked to the sequence encoding any of the nucleic acids (e.g., mRNAs) encoding the SpyCas9 nickase, the polymerase, or the fusion protein, or the guide RNAs described herein. In some embodiments, the DNA molecule is an expression construct suitable for expression in a mammalian cell, e.g., a human cell or a mouse cell, such as a human hepatocyte or a rodent (e.g., mouse) hepatocyte. In some embodiments, the DNA molecule is an expression construct suitable for expression in a cell of a mammalian organ, e.g., a human liver or a rodent (e.g., mouse) liver. In some embodiments, the DNA molecule is a plasmid or an episome. In some embodiments, the DNA molecule is contained in a host cell, such as a bacterium or a cultured eukaryotic cell. Exemplary bacteria include proteobacteria such as E. coli. Exemplary cultured eukaryotic cells include primary hepatocytes, including hepatocytes of rodent (e.g., mouse) or human origin; hepatocyte cell lines, including hepatocytes of rodent (e.g., mouse) or human origin; human cell lines; rodent (e.g., mouse) cell lines; Chinese hamster ovary (CHO) cells; microbial fungi, such as fission or budding yeasts, e.g., Saccharomyces, such as S. cerevisiae; and insect cells.

In some embodiments, a method of producing a nucleic acid (e.g., an mRNA or guide RNA) disclosed herein is provided. In some embodiments, such a method comprises contacting a DNA molecule described herein with an RNA polymerase under conditions permissive for transcription. In some embodiments, the contacting is performed in vitro, e.g., in a cell-free system. In some embodiments, the RNA polymerase is an RNA polymerase of bacteriophage origin, such as T7 RNA polymerase. In some embodiments, NTPs are provided that include at least one modified nucleotide as discussed above. In some embodiments, the NTPs include at least one modified nucleotide as discussed above and do not comprise UTP.

In some embodiments, a nucleic acid (e.g., an mRNA or guide RNA) disclosed herein alone or together with one or more guide RNAs, may be comprised within or delivered by a vector system of one or more vectors. In some embodiments, one or more of the vectors, or all of the vectors, may be DNA vectors. In some embodiments, one or more of the vectors, or all of the vectors, may be RNA vectors. In some embodiments, one or more of the vectors, or all of the vectors, may be circular. In other embodiments, one or more of the vectors, or all of the vectors, may be linear. In some embodiments, one or more of the vectors, or all of the vectors, may be enclosed in a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

Non-limiting exemplary viral vectors include adeno-associated virus (AAV) vectors, lentivirus vectors, adenovirus vectors, helper dependent adenoviral (HDAd) vectors, herpes simplex virus (HSV-1) vectors, bacteriophage T4, baculovirus vectors, and retrovirus vectors. In some embodiments, the viral vector may be an AAV vector. In other embodiments, the viral vector may be a lentivirus vector. In some embodiments, the lentivirus may be non-integrating. In some embodiments, the viral vector may be an adenovirus vector. In some embodiments, the adenovirus may be a high-cloning capacity or “gutless” adenovirus, where all coding viral regions apart from the 5′ and 3′ inverted terminal repeats (ITRs) and the packaging signal (‘I’) are deleted from the virus to increase its packaging capacity. In yet other embodiments, the viral vector may be an HSV-1 vector. In some embodiments, the HSV-1-based vector is helper dependent, and in other embodiments it is helper independent. For example, an amplicon vector that retains only the packaging sequence requires a helper virus with structural components for packaging, while a 30 kb-deleted HSV-1 vector that removes non-essential viral functions does not require helper virus. In additional embodiments, the viral vector may be bacteriophage T4. In some embodiments, the bacteriophage T4 may be able to package any linear or circular DNA or RNA molecules when the head of the virus is emptied. In further embodiments, the viral vector may be a baculovirus vector. In yet further embodiments, the viral vector may be a retrovirus vector. In embodiments using AAV or lentiviral vectors, which have smaller cloning capacity, it may be necessary to use more than one vector to deliver all the components of a vector system as disclosed herein. For example, one AAV vector may contain sequences encoding a Cas protein, while a second AAV vector may contain one or more guide sequences.

In some embodiments, the vector may be capable of driving expression of one or more coding sequences, such as the coding sequence of an mRNA disclosed herein, in a cell. In some embodiments, the cell may be a prokaryotic cell, such as, e.g., a bacterial cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the promoter may be constitutive, inducible, or tissue-specific. In some embodiments, the promoter may be a constitutive promoter. Non-limiting exemplary constitutive promoters include cytomegalovirus (CMV) immediate early promoter, simian virus (SV40) promoter, adenovirus major late promoter (MLP), Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EF1a promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In some embodiments, the promoter may be a tissue-specific promoter, e.g., a promoter specific for expression in the liver.

The vector may further comprise a nucleotide sequence encoding at least one guide RNA. In some embodiments, the vector comprises one copy of the guide RNA. In other embodiments, the vector comprises more than one copy of the guide RNA. In embodiments with more than one guide RNA, the guide RNAs may be non-identical such that they target different target sequences, or may be identical in that they target the same target sequence. In some embodiments where the vectors comprise more than one guide RNA, each guide RNA may have other different properties, such as activity or stability within a ribonucleoprotein complex with the SpyCas9 nickase or the fusion protein disclosed herein. In some embodiments, the nucleotide sequence encoding the guide RNA may be operably linked to at least one transcriptional or translational control sequence, such as a promoter, a 3′ UTR, or a 5′ UTR. In one embodiment, the promoter may be a tRNA promoter, e.g., tRNA^{Lys3}, or a tRNA chimera. See Mefferd et al., RNA. 2015 21:1683-9; Scherer et al., Nucleic Acids Res. 2007 35:2620-2628. In some embodiments, the promoter may be recognized by RNA polymerase III (Pol III). Non-limiting examples of Pol III promoters include U6 and H1 promoters. In some embodiments, the nucleotide sequence encoding the guide RNA may be operably linked to a mouse or human U6 promoter. In other embodiments, the nucleotide sequence encoding the guide RNA may be operably linked to a mouse or human H1 promoter. In embodiments with more than one guide RNA, the promoters used to drive expression may be the same or different. In some embodiments, the nucleotide sequence encoding the crRNA of the guide RNA and the nucleotide sequence encoding the trRNA of the guide RNA may be provided on the same vector. In some embodiments, the nucleotide sequence encoding the crRNA and the nucleotide sequence encoding the trRNA may be driven by the same promoter. In some embodiments, the crRNA and trRNA may be transcribed into a single transcript. For example, the crRNA and trRNA may be processed from the single transcript to form a double-molecule guide RNA. Alternatively, the crRNA and trRNA may be transcribed into a single-molecule guide RNA. In other embodiments, the crRNA and the trRNA may be driven by their corresponding promoters on the same vector. In yet other embodiments, the crRNA and the trRNA may be encoded by different vectors.

In some embodiments, the composition may comprise a vector system, wherein the system comprises more than one vector. In some embodiments, the vector system may comprise one single vector. In other embodiments, the vector system may comprise two vectors. In additional embodiments, the vector system may comprise three vectors. When multiple copies of the guide RNA are used, the vector system may comprise more than three vectors.

In some embodiments, the vector system may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the vector system may comprise tissue-specific promoters to start expression only after it is delivered into a specific tissue.

### Cells

In some embodiments, a cell contacted with the operably linked SpyCas9 nickase and polymerase disclosed herein is a human cell.

In some embodiments, a cell is treated in vitro with any method or composition disclosed herein. In some embodiments, a cell is treated in vivo with any method or composition disclosed herein.

In some embodiments, the cell in any of the embodiments provided herein is engineered by an operably linked SpyCas9 nickase and polymerase disclosed herein.

In some embodiments, the fusion protein disclosed herein is delivered to the cell via electroporation. In some embodiments, the operably linked SpyCas9 nickase and polymerase disclosed herein is delivered to the cell via at least one lipid nanoparticle (LNP). In some embodiments, the operably linked SpyCas9 nickase and polymerase disclosed herein is delivered to the cell on at least one vector. In some embodiments, the operably linked SpyCas9 nickase and polymerase disclosed herein is delivered as at least one nucleic acid encoding the fusion protein disclosed herein. In some embodiments, the at least one nucleic acid comprises at least one mRNA. In some embodiments, the operably linked SpyCas9 nickase and polymerase disclosed herein is delivered to the cell as at least one polypeptide or at least one mRNA. In some embodiments, the at least one guide RNA is delivered to the cell as at least one polynucleotide that encodes the guide RNA.

In some embodiments, the cell is an immune cell. As used herein, “immune cell” refers to a cell of the immune system, including e.g., a lymphocyte (e.g., T cell, B cell, natural killer cell (“NK cell”, and NKT cell, or iNKT cell)), monocyte, macrophage, mast cell, dendritic cell, or granulocyte (e.g., neutrophil, eosinophil, and basophil). In some embodiments, the cell is a primary immune cell. In some embodiments, the immune system cell may be selected from CD3⁺, CD4⁺ and CD8⁺ T cells, regulatory T cells (Tregs), B cells, NK cells, and dendritic cells (DC). In some embodiments, the immune cell is allogeneic.

In some embodiments, the cell is a lymphocyte. In some embodiments, the cell is an adaptive immune cell. In some embodiments, the cell is a T cell. In some embodiments, the cell is a B cell. In some embodiments, the cell is a NK cell.

In some embodiments, the cell is a mononuclear cell, such as from bone marrow or peripheral blood. In some embodiments, the cell is a peripheral blood mononuclear cell (“PBMC”). In some embodiments, the cell is a PBMC, e.g. a lymphocyte or monocyte. In some embodiments, the cell is a peripheral blood lymphocyte (“PBL”).

In some embodiments, the cell is derived from a progenitor cell before editing. In some embodiments, the cell is an induced pluripotent stem cell (iPSC).

Cells used in ACT therapy are included, such as mesenchymal stem cells (e.g., isolated from bone marrow (BM), peripheral blood (PB), placenta, umbilical cord (UC) or adipose); hematopoietic stem cells (HSCs; e.g. isolated from BM); mononuclear cells (e.g., isolated from BM or PB); endothelial progenitor cells (EPCs; isolated from BM, PB, and UC); neural stem cells (NSCs); limbal stem cells (LSCs); or tissue-specific primary cells or cells derived therefrom (TSCs). Cells used in ACT therapy further include induced pluripotent stem cells (iPSCs; see e.g., Mahla, International J. Cell Biol. 2016 (Article ID 6940283): 1-24 (2016)) that may be induced to differentiate into other cell types including e.g., islet cells, neurons, and blood cells; ocular stem cells; pluripotent stem cells (PSCs); embryonic stem cells (ESCs); cells for organ or tissue transplantations such as islet cells, cardiomyocytes, thyroid cells, thymocytes, neuronal cells, skin cells, retinal cells, chondrocytes, myocytes, and keratinocytes.

In some embodiments, the cell is a human cell, such as a cell from a subject. In some embodiments, the cell is isolated from a human subject. In some embodiments, the cell is isolated from a patient. In some embodiments, the cell is isolated from a donor. In some embodiments, the cell is isolated from human donor PBMCs or leukopaks. In some embodiments, the cell is from a subject with a condition, disorder, or disease. In some embodiments, the cell is from a human donor with Epstein Barr Virus (“EBV”).

In some embodiments, the methods disclosed herein are carried out ex vivo. As used herein, “ex vivo” refers to an in vitro method wherein the cell is capable of being transferred into a subject, e.g. as an ACT therapy. In some embodiments, an ex vivo method is an in vitro method involving an ACT therapy cell or cell population.

In some embodiments, the cell is maintained in culture. In some embodiments, the cell is transplanted into a patient. In some embodiments, the cell is removed from a subject, genetically modified ex vivo, and then administered back to the same patient. In some embodiments, the cell is removed from a subject, genetically modified ex vivo, and then administered to a subject other than the subject from which it was removed.

In some embodiments, the cell is from a cell line. In some embodiments, the cell line is derived from a human subject. In some embodiments, the cell line is a lymphoblastoid cell line (“LCL”). The cell may be cryopreserved and thawed. The cell may not have been previously cryopreserved.

In some embodiments, the cell is from a cell bank. In some embodiments, the cell is genetically modified and then transferred into a cell bank. In some embodiments the cell is removed from a subject, genetically modified ex vivo, and transferred into a cell bank. In some embodiments, a genetically modified population of cells is transferred into a cell bank. In some embodiments, a genetically modified population of immune cells is transferred into a cell bank. In some embodiments, a genetically modified population of immune cells comprising a first and second subpopulations, wherein the first and second sub-populations have at least one common genetic modification and at least one different genetic modification are transferred into a cell bank.

In some embodiments, a population of cells comprises any cell edited using any method or composition disclosed herein.

In some embodiments, a population of cells comprises edited T cells, and wherein at least 30%, 40%, 50%, 55%, 60%, 65% of the cells of the population have a memory phenotype (CD27+, CD45RA+).

In some embodiments, a population of cells comprises non-activated immune cells. In some embodiments, the population of cells comprises activated immune cells.

In some embodiments, a population of cells comprises T cells and is responsive to repeat stimulation after editing. In some embodiments, the population of cells is cultured, expanded, differentiated, or proliferated ex vivo.

In some embodiments, at least 5% of cells in a cell population comprise a templated edit following template-based genome editing. In some embodiments, at least 5%, 10%, 15%, 20%, 25%, 30%, 35%, 40%, 45%, optionally at least 50%, 55%, 60%, 65%, optionally at least 70%, 75%, 80%, 85%, 90%, of cells in a cell population comprise a templated edit following template-based genome editing. In some embodiments, the percentage of edited cells required to provide a therapeutic benefit depends, e.g., on the tissue and the disease to be treated.

TABLE 5A
Exemplary SpyCas9 sgRNA conserved portion (“Exemplary SpyCas9 sgRNA-1”; SEQ ID NO: 22)

| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 | 29 | 30 |
|---|---|---|---|---|---|---|---|---|---|---|---|---|---|---|---|---|---|---|---|---|---|---|---|---|---|---|---|---|---|
| G | U | U | U | U | A | G | A | G | C | U | A | G | A | A | A | U | A | G | C | A | A | G | U | U | A | A | A | A | U |
| LS1-LS6 | | | | | | B1-B2 | | US1-US12 | | | | | | | | | | | | B2-B6 | | | | LS7-LS12 | | | | | |
| 31 | 32 | 33 | 34 | 35 | 36 | 37 | 38 | 39 | 40 | 41 | 42 | 43 | 44 | 45 | 46 | 47 | 48 | 49 | 50 | 51 | 52 | 53 | 54 | 55 | 56 | 57 | 58 | 59 | 60 |
| A | A | G | G | C | U | A | G | U | C | C | G | U | U | A | U | C | A | A | C | U | U | G | A | A | A | A | A | G | U |

| Nexus | | | | | | | | | H1-1 through H1-12 | | | | | | |
|---|---|---|---|---|---|---|---|---|---|---|---|---|---|---|---|
| 61 | 62 | 63 | 64 | 65 | 66 | 67 | 68 | 69 | 70 | 71 | 72 | 73 | 74 | 75 | 76 |
| G | G | C | A | C | C | G | A | G | U | C | G | G | U | G | C |

| | |
|---|---|
| N | H2-1 through H2-15 |

TABLE 5B
Exemplary SpyCas9 sgRNA (SEQ ID NO: 115)

| 1-20 | 21 | 22 | 23 | 24 | 25 | 26 | 27 | 28 | 29 | 30 | 31 | 32 | 33 | 34 | 35 | 36 | 37 | 38 | 39 | 40 |
|---|---|---|---|---|---|---|---|---|---|---|---|---|---|---|---|---|---|---|---|---|
| NNNNNNNNNNNNNNNNNNNN | G | U | U | U | U | A | G | A | G | C | U | A | G | A | A | A | U | A | G | C |

| | | | |
|---|---|---|---|
| Spacer | LS1-LS6 | B1-B2 | US1-US12 |

| 41 | 42 | 43 | 44 | 45 | 46 | 47 | 48 | 49 | 50 | 51 | 52 | 53 | 54 | 55 | 56 | 57 | 58 | 59 | 60 | 61 | 62 | 63 | 64 | 65 | 66 | 67 | 68 |
|---|---|---|---|---|---|---|---|---|---|---|---|---|---|---|---|---|---|---|---|---|---|---|---|---|---|---|---|
| A | A | G | U | U | A | A | A | A | U | A | AG | | G | C | U | A | G | U | C | C | G | U | U | A | U | C | A |

| B2-B6 | | | | LS7-LS12 | | | | | | Nexus | | | | | | | | | | | | | | | | | |
|---|---|---|---|---|---|---|---|---|---|---|---|---|---|---|---|---|---|---|---|---|---|---|---|---|---|---|---|
| 69 | 70 | 71 | 72 | 73 | 74 | 75 | 76 | 77 | 78 | 79 | 80 | 81 | 82 | 83 | 84 | 85 | 86 | 87 | 88 | 89 | 90 | 91 | 92 | 93 | 94 | 95 | 96 |
| A | C | U | U | G | A | A | A | A | A | G | U | G | G | C | A | C | C | G | A | G | U | C | G | G | U | G | C |

| | | |
|---|---|---|
| H1-1 through H1-12 | N | H2-1 through H2-15 |

Lengthy table referenced here
US20260286329A1-20260924-T00001
Please refer to the end of the specification for access instructions.

### EXAMPLES

The following examples are provided to illustrate certain disclosed embodiments and are not to be construed as limiting the scope of this disclosure in any way.

### Example 1. Materials and Methods

The following materials and methods were utilized in the Examples provided herein, unless otherwise indicated.

1.1.1 In Vitro Transcription (“IVT”) of mRNA, and Production of Guide RNAs

Capped and polyadenylated mRNA containing N1-methyl pseudo-U was generated by in vitro transcription using a linearized plasmid DNA template and T7 RNA polymerase. The linearized plasmid DNA containing a T7 promoter, and a sequence for transcription was linearized by restriction endonuclease digestion followed by heat inactivation of the reaction mixture and purified from enzyme and buffer salts. Messenger RNA was synthesized and purified using standard techniques known in the art.

Messenger RNAs encoding enzymes or other polypeptides used in the experiments herein were generated from plasmid DNA encoding an open reading frame according to SEQ ID Nos: 1001-1573 (see sequences in Table 6). When the sequences cited in this paragraph are referred to below with respect to RNAs, it is understood that Ts should be replaced with Us (which can be modified nucleosides as described above). Messenger RNAs used in the Examples include a 5′ cap and a 3′ polyadenylation sequence e.g., up to 100 nts. Guide RNAs were chemically synthesized by commercial vendors or using standard in vitro synthesis techniques with modified nucleotides, unless otherwise indicated in the below examples.

### 1.2 Lipid Nanoparticle Formulation

In general, the lipid nanoparticle (LNP) components were dissolved in 100% ethanol at various molar ratios. The RNA cargos (e.g., SpyCas9 nickase mRNA, guide RNA) were dissolved in 25 mM citrate, 100 mM NaCl, pH 5.0, resulting in a concentration of RNA cargo of approximately 0.45 mg/mL. The LNPs used contained an ionizable lipid (e.g., amine lipid) (e.g., (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), also called herein Lipid A, Lipid B, Lipid C, Lipid D, Lipid E, or Lipid F, cholesterol, distearoylphosphatidylcholine (DSPC), and a PEG lipid (e.g., 1,2-dimyristoyl-rac-glycero-3-methoxypolyethylene glycol-2000 (PEG2k-DMG) (catalog #GM 020 from NOF, Tokyo, Japan), or C13 Ether) in a molar ratio of 50% ionizable lipid, 38% cholesterol, 9% DSPC, and 3% PEG (e.g., PEG-2k DMG) lipid, unless otherwise indicated. The LNPs were formulated with a lipid amine to RNA phosphate (N: P) molar ratio of about 6. The LNPs used comprised a single RNA species such a mRNA or a guide RNA. LNPs were similarly prepared with a mixture of mRNA and guide RNA.

The LNPs were prepared using a cross-flow technique utilizing impinging jet mixing of the lipid in ethanol with two volumes of RNA solution and one volume of water. First, the lipid in ethanol was mixed through a mixing cross with the two volumes of RNA solution. Then, a fourth stream of water was mixed with the outlet stream of the cross through an inline tee (See WO2016010840 FIG. 2). The LNPs were held for at least 1 hour at room temperature, and further diluted with water (approximately 1:1 v/v). Diluted LNPs were buffer exchanged into 50 mM Tris, 45 mM NaCl, 5% (w/v) sucrose, pH 7.5 (TSS) and concentrated as needed by methods known in the art. The resulting mixture was then filtered using a 0.2 μm sterile filter. The final LNPs were characterized to determine the encapsulation efficiency, polydispersity index, and average particle size. The final LNP was stored at 4° C. or −80° C. until further use.

### 1.3 Next-Generation Sequencing (“NGS”) and Analysis for Editing Efficiency

DNA was extracted using a commercial kit according to the manufacturer's protocol, for example QuickExtract™ DNA Extraction Solution (Lucigen, Cat. QE09050). To quantitatively determine the efficiency of editing at the target location in the genome, deep sequencing was utilized to identify the presence of insertions and deletions introduced by gene editing. PCR primers were designed around the target site within the gene of interest (e.g., HEK3) and the genomic area of interest was amplified. Primer sequence design was done as is standard in the field. Additional PCR was performed according to the manufacturer's protocols (e.g., Illumina, PacBio) to add chemistry for sequencing. The amplicons were sequenced on a NGS platform (e.g., Illumina, PacBio). The reads were aligned to the reference genome (e.g., hg38) after eliminating those having nucleotides with low quality scores (PHRED score <20, unless otherwise indicated). Reads that overlapped the target sequence of interest were re-aligned to the local genome sequence to improve the alignment.

### 1.3.1 Indel Analysis

The number of wild-type reads versus the number of reads which contain indels was calculated. Insertions and deletions were scored in a ~20 bp region centered on the predicted SpyCas9 cleavage or nicking site. Indel percentage is defined as the total number of sequencing reads with one or more base inserted or deleted within the ~20 bp scoring region divided by the total number of sequencing reads, including wild type.

### 1.3.2 Editing Analysis

To quantify targeted repair, each read was aligned to a reference sequence with the desired templated edit(s) (e.g., G-to-C transversion, GAGG-to-CACT edit, insertion or deletion) in the middle, plus about 45-80 nt upstream and about 45-80 nt downstream from the target genomic locus (Smith & Waterman, 1981; Zhao et al., 2013). Reads aligned to this reference sequence without mismatches or indels were classified as templated editing or templated repair, disregarding end-clippings. The “templated editing” percentage was defined as the total number of templated editing reads divided by the total number of sequencing reads inspected.

Reads comprising more than 50% and less than 100% of the templated edit(s) and reads comprising the templated edit(s) and one or more byproduct mismatches or indels (i.e., mismatches or indels not encoded in the template) were counted as reads with “templated editing+byproduct editing”, and the corresponding percentage was calculated as the number of reads with “templated editing+byproduct editing” divided by the total number of sequencing reads inspected.

When one or more mismatches (relative to the wild-type genome) were introduced in the DRS region of the 3′ extension of the guide RNA, reads that comprised mismatches (relative to the reference sequence) that were complementary to the DRS mismatch(es) were counted as “DRS mismatch editing” and the percentage corresponding to the “templated editing+DRS mismatch editing” was calculated as the number of reads with both the templated edit(s) and the edit(s) complementary to the DRS mismatch(es) divided by the total number of sequencing reads inspected.

When the template region of the 3′ extension of the guide RNA encoded an insertion or a deletion edit, reads with the encoded insertion or deletion were counted towards the “templated edits” percentage as described above, and the “non-templated indels” were calculated as the subtraction of the “templated edits” as described above from the “indels” count as described in Example 1.3.1.

### 1.4. Guide RNA and Cas9 mRNA Lipofection

Lipofection of mRNA and guide RNAs used pre-mixed lipid formulations. The lipofection reagent contained ionizable Lipid A, cholesterol, DSPC, and PEG2k-DMG in a molar ratio of 50% Lipid A, 38% cholesterol, 9% DSPC, and 3% PEG2k-DMG, unless indicated otherwise. This mixture was reconstituted in 100% ethanol then mixed with RNA (e.g., SpyCas9 nickase mRNA and guide RNA) at a lipid amine to RNA phosphate (N: P) molar ratio ranging from about 6 to about 50, unless otherwise indicated, to produce lipid nucleic acid assemblies.

Lipofections were performed with a ratio of guide RNA to mRNA of 1:2 by weight, unless otherwise indicated. Briefly, cells were incubated at 37° C., 5% CO₂ for 24 hours prior to treatment with the lipid nucleic acid mixtures. Lipid nucleic acid mixtures were incubated in media containing 10% fetal bovine serum (FBS) at 37° C. for 10 minutes. Post-incubation, the lipid nucleic acid mixtures were added to the cells (e.g., mouse, cynomolgus, or human hepatocytes, T cells, or hepatoma cells) in a 6 or 12 point 3-fold dilution series starting at 300 ng SpyCas9 nickase mRNA, unless otherwise indicated. The cells were lysed 72 hours post-treatment for NGS analysis as described in Example 1.

### 1.5. Huh7 Cell Preparation

Cells were thawed in complete media containing 1× Dulbecco's Modification of Eagle's Medium with 4.5 g/L glucose, L-glutamine & sodium pyruvate (Corning, Cat. 10-013-CV), FBS (Gibco, Cat. A13450) at 10%, and Penstrep (Corning, Cat. 30-002-CI) at 1% and passaged following centrifugation and resuspension. Cells were trypsinized, then counted and plated on Collagen I Cellware 96-well plates (e.g., Corning, Cat. 354407; Thermo Fisher, Cat. 877272; or StemCell Technologies, Cat. 100-0366) at a density of 15,000 cells/well. Cells were incubated in a tissue culture incubator at 37° C. and 5% CO₂ atmosphere for 24 hours.

### 1.6. Primary Human Hepatocyte, Primary Cynomolgus Hepatocyte, and Primary Mouse Hepatocyte Preparation

Primary human hepatocytes (e.g., Gibco, Lot 8381), primary cynomolgus hepatocytes (e.g., Gibco, Lot CY423), or primary mouse hepatocytes (e.g., Gibco, Lot MCM855) were thawed and resuspended in Cryopreserved Hepatocyte Recovery Medium (Invitrogen, Cat. CM7000) followed by centrifugation. The supernatant was discarded, and the pelleted cells were resuspended in hepatocyte plating medium (Gibco Williams' Medium E, Cat. A12176-01) plus supplement pack (Gibco, Cat. A15563 CM3000) and 5% FBS (Gibco, A13450). Cells were counted and plated on Bio-coat collagen I coated 96-well plates (Thermo Fisher, Cat. 877272; or StemCell Technologies, Cat. 100-0366) at a density of 35,000 cells/well. Plated cells were allowed to settle and adhere for 4 hours in a tissue culture incubator at 37° C. and 5% CO₂ atmosphere. After incubation, cells were checked for monolayer formation and washed once with hepatocyte maintenance medium (Gibco Williams' Medium E, Cat. A12176-01) plus cell maintenance supplements (Gibco, Cat. A15564 CM4000).

### 1.7 Modified Nucleotides Used in Examples

Table 7 lists the indicators of nucleotide modification and linkers in the context of modified polynucleotides that are used in tables below in these examples.

TABLE 7
Indicators representing modified nucleotides

| Indicator | Definition |
|---|---|
| A | Adenosine ribonucleotide |
| C | Cytosine ribonucleotide |
| G | Guanosine ribonucleotide |
| U | Uridine ribonucleotide |
| mA | 2′O-methyl adenosine nucleotide |
| mC | 2′O-methyl cytosine nucleotide |
| mG | 2′O-methyl guanosine nucleotide |
| mU | 2′O-methyl uridine nucleotide |
| * | 3′ phosphorothioate linkage between nucleotides |
| dA | Adenosine deoxynucleotide |
| dC | Cytosine deoxynucleotide |
| dG | Guanosine deoxynucleotide |
| dT | Thymidine deoxynucleotide |
| fA | 2′F adenosine nucleotide |
| fC | 2′F cytosine nucleotide |
| fG | 2′F guanosine nucleotide |
| fU | 2′F uridine nucleotide |
| C7NH2 | 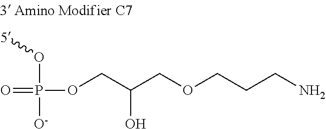 |
| L4 (or S3) internal linker | 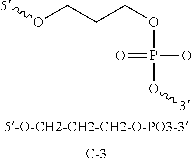 |
| L3 (or S6) internal linker | 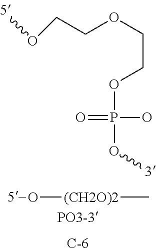 |
| L2 (or S9) internal linker | 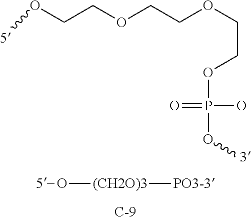 |
| S12 internal linker | 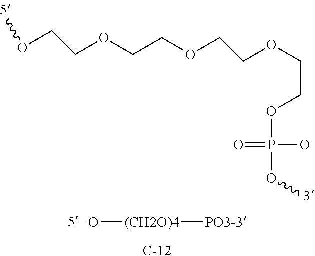 |
| S15 internal linker | 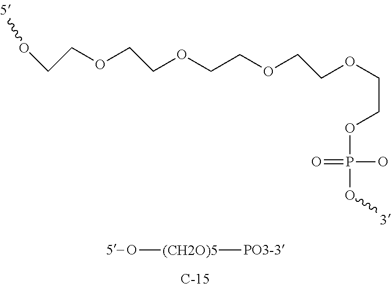 |
| L1 (or S18) internal linker | 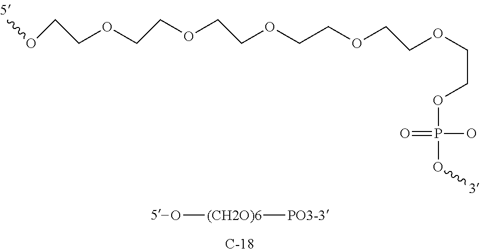 |

### Example 2—Dose Response Experiment with Truncated Guide RNAs in Huh7 Cells

This Example evaluated the editing efficiency of template guide RNAs (tgRNAs) complexed with SpyCas9. In particular, this study evaluated tgRNAs with a 3′ extension encoding a base edit as compared to (i) guide RNAs without a 3′ extension; (ii) guide RNAs having different truncation lengths in the flagpole region (also referred as upper stem region elsewhere herein); or (iii) guide RNAs with various internal linkers as described in Table 7. Each guide RNA was complexed with SpyCas9 and assessed for in vitro editing via a dose response experiment in human hepatoma (Huh7) cells. See Table 8A for a description of each guide RNA assessed in this Example.

Guide RNAs targeting the same exemplary/proof of concept target sequence in the HEK3 genomic locus, with a 3′ extension (i.e., a tgRNA) or without a 3′ extension encoding a G-to-C edit in the genomic DNA, were designed with different truncation lengths in the guide RNA flagpole region (also referred to as upper stem region elsewhere herein), and variations in internal linkers as shown in Table 7. The guide RNAs were lipofected into human hepatoma (Huh7) cells to determine guide potency as follows.

Cells were prepared as described in Example 1, plated at a density of 15,000 cells/well, and incubated in a tissue culture incubator at 37° C. and 5% CO₂ atmosphere for 24 hours. Lipofectamine™ MessengerMAX™ Reagent (Invitrogen, Cat. LMRNA015) was used for lipofection and samples were prepared according to the manufacturer's protocol with 50 ng of SpyCas9 mRNA (SEQ ID NO: 1001) per reaction and an initial 50 nM guide concentration. Each guide RNA was diluted in Opti-MEM™ (Gibco, Cat. 31985-062) in a 5-fold, 6-point dilution series starting at 50 nM guide RNA prior to adding 0.3 μL of lipoplex. Samples were tested in duplicates. The cells were lysed 72 hours post-treatment for NGS analysis as described in Example 1.

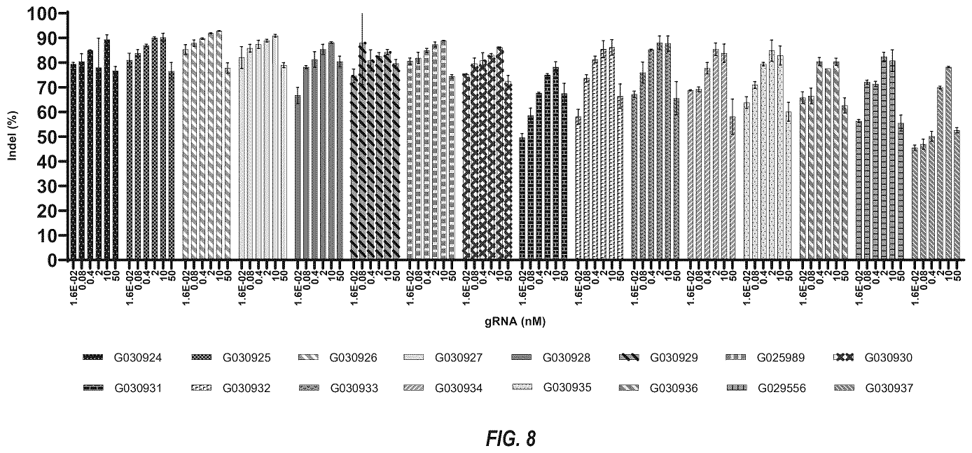

Mean percent indel results with standard deviation (SD) are shown in Table 8B and FIG. 8.

TABLE 8A
Description of guide RNA Constructs

| Guide ID | Scaffold structure (if a 3′ extension is included, it is shown in italic) |
|---|---|
| G030924 | GUUUUAGA(dS)AAGUUAAAAUAAGGCUAGUCCGUUAUCAC(L1)GGGCACCG |
| (SEQ ID | AGUCGGmU*mG*mC*mU (SEQ ID NO: 439) |
| NO: 401) | |
| G030925 | GUUUUAGA(L4)AAGUUAAAAUAAGGCUAGUCCGUUAUCAC(L1)GGGCACCG |
| (SEQ ID | AGUCGGmU*mG*mC*mU (SEQ ID NO: 440) |
| NO: 402) | |
| G030926 | GUUUUAGA(L3)AAGUUAAAAUAAGGCUAGUCCGUUAUCAC(L1)GGGCACCG |
| (SEQ ID | AGUCGGmU*mG*mC*mU (SEQ ID NO: 441) |
| NO: 403) | |
| G030927 | GUUUUAGA(L2)AAGUUAAAAUAAGGCUAGUCCGUUAUCAC(L1)GGGCACCG |
| (SEQ ID | AGUCGGmU*mG*mC*mU |
| NO: 404) | |
| G030928 | GUUUUAGA(LI)AAGUUAAAAUAAGGCUAGUCCGUUAUCAC(L1)GGGCACCG |
| (SEQ ID | AGUCGGmU*mG*mC*mU |
| NO: 405) | |
| G030929 | GUUUUAGAmGmC(L1)mGmCAAGUUAAAAUAAGGCUAGUCCGUUAUCAC(L1) |
| (SEQ ID | GGGCACCGAGUCGGmU*mG*mC*mU |
| NO: 406) | |
| G025989 | GUUUUAGAmGmCmUmA(L1)mUmAmGmCAAGUUAAAAUAAGGCUAGUCCGU |
| (SEQ ID | UAUCAC(L1)GGGCACCGAGUCGGmU*mG*mC*mU |
| NO: 407) | |
| G030930 | GUUUUAGAmGmCmUmA(L1)mUmAmGmCAAGUUAAAAUAAGGCUAGUCCGU |
| (SEQ ID | UAUCAC(L1)GGGCACCGAGUmCmGmGmU*mG*mC*mU |
| NO: 408) | |
| G030931 | GUUUUAGA(dS)AAGUUAAAAUAAGGCUAGUCCGUUAUCAC(L1)GGGCACCG |
| (SEQ ID | AGUCGGmUmGmCUCUGCGAUCACGUGCmUmCmAmGmUmCmU*mG |
| NO: 409) | |
| G030932 | GUUUUAGA(L4)AAGUUAAAAUAAGGCUAGUCCGUUAUCAC(L1)GGGCACCG |
| (SEQ ID | AGUCGGmUmGmCUCUGCGAUCACGUGCmUmCmAmGmUmCmU*mG |
| NO: 410) | |
| G030933 | GUUUUAGA(L3)AAGUUAAAAUAAGGCUAGUCCGUUAUCAC(L1)GGGCACCG |
| (SEQ ID | AGUCGGmUmGmCUCUGCGAUCACGUGCmUmCmAmGmUmCmU*mG |
| NO: 411) | |
| G030934 | GUUUUAGA(L2)AAGUUAAAAUAAGGCUAGUCCGUUAUCAC(S18)GGGCACCG |
| (SEQ ID | AGUCGGmUmGmCUCUGCGAUCACGUGCmUmCmAmGmUmCmU*mG |
| NO: 412) | |
| G030935 | GUUUUAGA(L1)AAGUUAAAAUAAGGCUAGUCCGUUAUCAC(S18)GGGCACCG |
| (SEQ ID | AGUCGGmUmGmCUCUGCGAUCACGUGCmUmCmAmGmUmCmU*mG |
| NO: 413) | |
| G030936 | GUUUUAGAmGmC(L1)mGmCAAGUUAAAAUAAGGCUAGUCCGUUAUCAC |
| (SEQ ID | (S18)GGGCACCGAGUCGGmUmGmCUCUGCGAUCACGUGCmUmCmAmGmUmCmU* |
| NO: 414) | mG |
| G029556 | GUUUUAGAmGmCmUmA(L1)mUmAmGmCAAGUUAAAAUAAGGCUAGUCCGU |
| (SEQ ID | UAUCAC(L1)GGGCACCGAGUCGGmUmGmCUCUGCGAUCACGUGCmUmCmAm |
| NO: 415) | GmUmCmU*mG |
| G030937 | GUUUUAGAmGmCmUmA(L1)mUmAmGmCAAGUUAAAAUAAGGCUAGUCCGU |
| (SEQ ID | UAUCAC(L1)GGGCACCGAGUmCmGmGmUmGmCUCUGCGAUCACGUGCmUmC |
| NO: 416) | mAmGmUmCmU*mG |

TABLE 8B
Mean percent indels measured at the HEK3 locus with
the delivery of the SpyCas9 mRNA in Huh7 cells.

| Guide ID | Guide RNA (nM) | Mean Indels (%) | SD |
|---|---|---|---|
| G030924 | 50 | 76.65 | 1.77 |
| | 10 | 89.35 | 1.91 |
| | 2 | 78.00 | 11.88 |
| | 0.4 | 84.95 | 0.21 |
| | 0.08 | 80.45 | 3.18 |
| | 0.016 | 79.40 | 0.71 |
| G030925 | 50 | 76.50 | 3.54 |
| | 10 | 90.20 | 1.70 |
| | 2 | 90.00 | 0.42 |
| | 0.4 | 86.95 | 0.50 |
| | 0.08 | 83.75 | 1.49 |
| | 0.016 | 81.00 | 2.83 |
| G030926 | 50 | 77.80 | 2.12 |
| | 10 | 92.80 | 0.14 |
| | 2 | 91.85 | 0.21 |
| | 0.4 | 89.70 | 0.28 |
| | 0.08 | 87.90 | 1.27 |
| | 0.016 | 85.30 | 1.98 |
| G030927 | 50 | 79.00 | 0.99 |
| | 10 | 90.90 | 0.57 |
| | 2 | 88.90 | 0.57 |
| | 0.4 | 87.40 | 1.70 |
| | 0.08 | 85.80 | 1.56 |
| | 0.016 | 82.05 | 4.46 |
| G030928 | 50 | 80.45 | 2.19 |
| | 10 | 88.15 | 0.35 |
| | 2 | 85.35 | 2.05 |
| | 0.4 | 81.25 | 3.18 |
| | 0.08 | 78.15 | 0.64 |
| | 0.016 | 66.70 | 3.25 |
| G030929 | 50 | 79.65 | 1.63 |
| | 10 | 84.25 | 1.06 |
| | 2 | 82.55 | 1.49 |
| | 0.4 | 80.85 | 4.31 |
| | 0.08 | 88.00 | 16.97 |
| | 0.016 | 74.60 | 2.83 |
| G025989 | 50 | 74.40 | 0.71 |
| | 10 | 88.90 | 0.14 |
| | 2 | 87.35 | 1.06 |
| | 0.4 | 84.90 | 0.85 |
| | 0.08 | 81.90 | 2.26 |
| | 0.016 | 80.60 | 1.27 |
| G030930 | 50 | 72.45 | 2.33 |
| | 10 | 86.15 | 0.21 |
| | 2 | 83.05 | 0.64 |
| | 0.4 | 80.90 | 3.11 |
| | 0.08 | 79.60 | 2.26 |
| | 0.016 | 75.25 | 0.21 |
| G030931 | 50 | 67.45 | 4.17 |
| | 10 | 78.15 | 2.19 |
| | 2 | 75.05 | 0.50 |
| | 0.4 | 67.65 | 0.35 |
| | 0.08 | 58.55 | 3.04 |
| | 0.016 | 49.65 | 1.63 |
| G030932 | 50 | 66.40 | 4.95 |
| | 10 | 86.20 | 3.11 |
| | 2 | 85.45 | 3.46 |
| | 0.4 | 81.25 | 1.34 |
| | 0.08 | 73.65 | 1.49 |
| | 0.016 | 58.10 | 2.97 |
| G030933 | 50 | 65.55 | 6.72 |
| | 10 | 87.70 | 3.11 |
| | 2 | 88.00 | 2.83 |
| | 0.4 | 85.25 | 0.21 |
| | 0.08 | 75.95 | 4.31 |
| | 0.016 | 67.15 | 1.34 |
| G030934 | 50 | 58.05 | 7.14 |
| | 10 | 83.80 | 3.68 |
| | 2 | 85.40 | 2.55 |
| | 0.4 | 77.75 | 2.33 |
| | 0.08 | 69.20 | 0.99 |
| | 0.016 | 68.75 | 0.21 |
| G030935 | 50 | 60.10 | 3.82 |
| | 10 | 82.85 | 3.89 |
| | 2 | 84.85 | 4.31 |
| | 0.4 | 79.40 | 0.71 |
| | 0.08 | 70.95 | 1.34 |
| | 0.016 | 63.80 | 2.40 |
| G030936 | 50 | 62.65 | 3.04 |
| | 10 | 80.35 | 1.49 |
| | 2 | 77.60* | N/A |
| | 0.4 | 80.45 | 1.63 |
| | 0.08 | 66.45 | 3.18 |
| | 0.016 | 65.80 | 2.40 |
| G029556 | 50 | 55.50 | 3.25 |
| | 10 | 80.80 | 4.38 |
| | 2 | 82.45 | 1.77 |
| | 0.4 | 71.35 | 1.06 |
| | 0.08 | 72.10 | 0.85 |
| | 0.016 | 56.35 | 0.64 |
| G030937 | 50 | 52.55 | 1.06 |
| | 10 | 78.20 | 0.28 |
| | 2 | 69.90 | 0.57 |
| | 0.4 | 50.15 | 2.05 |
| | 0.08 | 46.95 | 2.05 |
| | 0.016 | 45.55 | 1.06 |

*Indicates an experimental group for which N = 1

### Example 3—DNA-Templated Editing in Huh7 Cells with Various DNA/RNA 3′ Extensions

This Example evaluated templated editing efficiency with tgRNAs having a separable 3′ extension sequence (e.g., a 3′ extension including a template sequence and a DNA polymerase recruiting sequence (DRS)). When the 3′ extension is not covalently attached to the 3′ end of the scaffold, it is referred to herein as a separable 3′ extension. The separable 3′ extension can be retained at the site of template-based genome editing by binding to the DNA polymerase, e.g., via an affinity tag (e.g., an aptamer, such as an MS2 loop, also known herein as an MS2 hairpin, MS2 aptamer, and MS2 RNA loop) to a 3′ extension-recruiting domain in the polymerase (e.g., MS2 coat protein; MCP).

Further, the impact of the DNA/RNA composition in the template sequence and DRS sequence of the separable 3′ extension was assessed. Each guide RNA was complexed with a SpyCas9 nickase (nCas9)-DNA polymerase fusion protein that further included a tag (3′ extension-recruiting domain) capable of binding the aptamer on the tgRNA (e.g., a MS2 coat protein) and assessed for in vitro templated editing in human hepatoma (Huh7) cells. See Table 9 for a description of the guide RNAs evaluated in this Example.

Polynucleotides were designed for delivery of a first polynucleotide being an sgRNA targeting and exemplary target locus (e.g., VEGFA), and a second polynucleotide being an MS2-loop-linked 3′-extension, i.e., a separable 3′ extension, including, 5′ to 3′, an aptamer sequence (MS2 loop; SEQ ID NO: 601) for binding to a SpyCas9 nickase-DNA polymerase fusion protein, a template sequence encoding a GAGG-to-CACT edit in the genomic locus, and a DRS. Each of the MS2-loop-linked 3′-extensions had a different RNA/DNA composition, as shown in Table 9. The sgRNA was paired with each of the MS2-loop-linked 3′-extension and delivered to human hepatoma cells (Huh7) along with mRNAs encoding a H840A SpyCas9 nickase fused to an MS2 coat protein (MCP; SEQ ID NO: 1519) and a DNA-dependent DNA polymerase. An additional nicking guide RNA (G034622; SEQ ID NO: 527), which targeted the opposite strand of the tgRNA 90 bp away from the cut site, was also used.

TABLE 9
Exemplary sequences of MS2-loop-linked separable 3′ extensions

| MS2-loop- | |
|---|---|
| linked 3′ | |
| extension ID | Sequence (template in italic, DRS in bold, and MS2-loop in plain text) |
| ST034637 | mG*mC*mAmCmAmUGAmGmGAUCAmCmCmCmAmUmGmUmGmC |
| (SEQ ID NO: | dTdGdCdCdAdTdCdTdGdGdAdGdCdAdGUGAUCUGGmCmCmUmGmCmAmG |
| 483) | mAmCmAmU*mC |
| ST034638 | mG*mC*mAmCmAmUGAmGmGAUCAmCmCmCmAmUmGmUmGmCdTdGdCd |
| (SEQ ID NO: | CdAdTdCdTdGdGdAdGdCdAdGdTGAUCUGGmCmCmUmGmCmAmGmAmCm |
| 484) | AmU*mC |
| ST034639 | mG*mC*mAmCmAmUGAmGmGAUCAmCmCmCmAmUmGmUmGmCdTdGdCd |
| (SEQ ID NO: | CdAdTdCdTdGdGdAdGdCdAdGdTdGAUCUGGmCmCmUmGmCmAmGmAmC |
| 485) | mAmU*mC |
| ST034640 | mG*mC*mAmCmAmUGAmGmGAUCAmCmCmCmAmUmGmUmGmCdTdGdCd |
| (SEQ ID NO: | CdAdTdCdTdGdGdAdGdCdAdGdTdGdAUCUGGmCmCmUmGmCmAmGmAm |
| 486) | CmAmU*mC |
| ST034641 | mG*mC*mAmCmAmUGAmGmGAUCAmCmCmCmAmUmGmUmGmCdTdGdCd |
| (SEQ ID NO: | CdAdTdCdTdGdGdAdGdCdAdGdTdGdAdTCUGGmCmCmUmGmCmAmGmAm |
| 487) | CmAmU*mC |
| ST034642 | mG*mC*mAmCmAmUGAmGmGAUCAmCmCmCmAmUmGmUmGmCdTdGdCd |
| (SEQ ID NO: | CdAdTdCdTdGdGdAdGdCdAdGdTdGdAdTdCUGGmCmCmUmGmCmAmGmA |
| 488) | mCmAmU*mC |
| ST034643 | mG*mC*mAmCmAmUGAmGmGAUCAmCmCmCmAmUmGmUmGmCdTdGdCd |
| (SEQ ID NO: | CdAdTdCdTdGdGdAdGdCdAdGdTdGdAdTdCdTGGmCmCmUmGmCmAmGm |
| 489) | AmCmAmU*mC |
| ST034644 | mG*mC*mAmCmAmUGAmGmGAUCAmCmCmCmAmUmGmUmGmCdTdGdCd |
| (SEQ ID NO: | CdAdTdCdTdGdGdAdGdCdAdGdTdGdAdTdCdTdGdGmCmCmUmGmCmAmG |
| 490) | mAmCmAmU*mC |
| ST034645 | mG*mC*mAmCmAmUGAmGmGAUCAmCmCmCmAmUmGmUmGmCdTdGdCd |
| (SEQ ID NO: | CdAdTdCdTdGdGdAdGdCdAdGdTdGdAdTdCdTdGdGdCdCmUmGmCmAmGm |
| 491) | AmCmAmU*mC |
| ST034828 | mG*mC*mAmCmAmUGAmGmGAUCAmCmCmCmAmUmGmUmGmCUGCCA |
| (SEQ ID NO: | UCUGGAGCAGUGAUCUGGmCmCmUmGmCmAmGmAmCmAmU*mC |
| 492) | |

Note:
*denotes that the nucleotide is linked to the next nucleotide with a phosphorothioate (PS bond), a lower case “m” denotes that the nucleotide is modified with 2′-O-Me, and a lowercase “d” denotes that the nucleotide is a DNA nucleotide.

Cells were prepared as described in Example 1, plated at a density of 15,000 cells/well and incubated at 37° C. and 5% CO₂ atmosphere for 24 hours.

mRNAs and guides were delivered using Lipofectamine™ MessengerMAX™ Reagent (Thermo Fisher, Cat. LMRNA001) according to the manufacturer's protocol. Lipoplexes comprising 100 ng of each of the different mRNAs were formed. Lipoplexes containing a mixture of 25 nM sgRNA (G034491; SEQ ID NO: 482) and 25 nM MS2-loop-linked 3′-extension were also formed. mRNA lipoplexes and guide lipoplexes were then delivered simultaneously. Samples were run in duplicates.

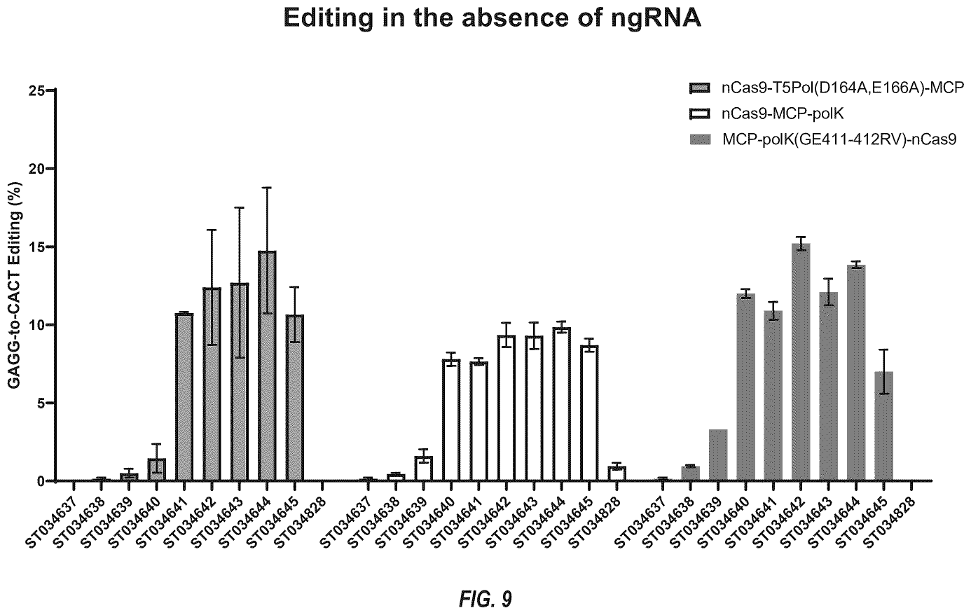

Cells were lysed 3 days post-treatment for NGS analysis as described in Example 1. Mean percent templated editing with standard deviation (SD) are shown in Tables 10 and 11 and FIGS. 9 and 10.

TABLE 10
Templated GAGG-to-CACT editing at the VEGFA locus with sgRNA (G034491) and MS2-loop-
linked 3′-extensions comprising various RNA/DNA compositions in the absence of ngRNA

| | mRNA ID | | | | | |
|---|---|---|---|---|---|---|
| | nCas9-T5Pol(D164A, E166A)-MCP | | nCas9-MCP-polK | | MCP-polK(GE411-412RV)-nCas9 | |
| | (SEQ ID NO: 1168) | | (SEQ ID NO: 1049) | | (SEQ ID NO: 1054) | |
| | Mean % | | Mean % | | Mean % | |
| Guide ID | Editing | SD | Editing | SD | Editing | SD |
| ST034637 | 0.00 | 0.00 | 0.15 | 0.07 | 0.15 | 0.07 |
| ST034638 | 0.15 | 0.07 | 0.45 | 0.07 | 0.95 | 0.07 |
| ST034639 | 0.50 | 0.28 | 1.60 | 0.42 | 3.30 | 0.00 |
| ST034640 | 1.45 | 0.92 | 7.80 | 0.42 | 12.00 | 0.28 |
| ST034641 | 10.75 | 0.07 | 7.65 | 0.21 | 10.90 | 0.57 |
| ST034642 | 12.40 | 3.68 | 9.35 | 0.78 | 15.20 | 0.42 |
| ST034643 | 12.70 | 4.81 | 9.30 | 0.85 | 12.10 | 0.85 |
| ST034644 | 14.75 | 4.03 | 9.85 | 0.35 | 13.85 | 0.21 |
| ST034645 | 10.65 | 1.77 | 8.70 | 0.42 | 7.00 | 1.41 |
| ST034828 | 0.00 | 0.00 | 0.95 | 0.21 | 0.00 | 0.00 |

TABLE 11
Templated GAGG-to-CACT editing at the VEGFA locus with sgRNA (G034491) and MS2-loop-linked
3′-extensions comprising various RNA/DNA compositions in the presence of ngRNA (G034622)

| | mRNA ID | | | | | |
|---|---|---|---|---|---|---|
| | nCas9-T5Pol(D164A,E166A)-MCP | | nCas9-MCP-polK | | MCP-polK(GE411-412RV)-nCas9 | |
| | (SEQ ID NO: 1168) | | (SEQ ID NO: 1049) | | (SEQ ID NO: 1054) | |
| | Mean % | | Mean % | | Mean % | |
| Guide ID | editing | SD | editing | SD | editing | SD |
| ST034637 | 0.00 | 0.00 | 0.50 | 0.42 | 0.35 | 0.07 |
| ST034638 | 0.10 | 0.00 | 2.10 | 0.28 | 2.85 | 0.21 |
| ST034639 | 0.10 | 0.00 | 5.45 | 1.06 | 8.65 | 1.20 |
| ST034640 | 0.90 | 0.14 | 14.85 | 2.33 | 18.05 | 0.21 |
| ST034641 | 12.85 | 2.90 | 20.60 | 5.52 | 19.40 | 0.99 |
| ST034642 | 14.25 | 3.61 | 22.40 | 4.95 | 22.10 | 1.13 |
| ST034643 | 18.80 | 7.21 | 23.35 | 6.01 | 23.30 | 0.99 |
| ST034644 | 22.60 | 5.09 | 19.55 | 8.98 | 21.75 | 0.07 |
| ST034645 | 16.00 | 5.23 | 18.15 | 3.46 | 14.85 | 0.21 |

*indicates an experimental group for which N = 1

### Example 4—Editing with MS2-Loop-Linked 3′ Extensions in Huh7 Cells with Various Types and Positions of Base Edits, and Template Lengths for Various Length Insertions or Deletions

This Example evaluated templated editing efficiency with tgRNAs having a separable 3′ extension sequence (e.g., including a template sequence and a DNA polymerase recruiting sequence (DRS)). Further, this Example assessed the impact of the position of the base edit in the template sequence of the tgRNA (e.g., at any one of positions T1-T15), the type of base edit encoded in the tgRNA (e.g., base transition or transversion, insertion, or deletion), and the length of the template sequence. Each guide RNA was complexed with a SpyCas9 nickase-DNA polymerase fusion protein that further included an MS2 coat protein and assessed for in vitro templated editing in human hepatoma (Huh7) cells. See Table 12 for a description of the guide RNAs evaluated in this Example.

Guide RNAs targeting the HEK3 locus were designed, comprising two separate polynucleotides. The first polynucleotide was an sgRNA (G033301) comprising a spacer sequence targeting HEK3 to recruit the SpyCas9 protein at the HEK3 locus. The second polynucleotide was an MS2-loop-linked 3′-extension comprising an MS2 loop aptamer sequence (SEQ ID NO: 601) and, 3′ of the MS2 loop, a 3′-extension that comprised a template sequence and a DRS targeting HEK3, as listed in Table 12. Some MS2-loop-linked 3′-extension encoded a base transition (A-to-G, G-to-A, C-to-T, T-to-C) or transversion (A-to-C, C-to-A, G-to-T, T-to-G, A-to-T, T-to-A, G-to-C, C-to-G) edit in the genomic DNA and varied in the position of the specific transition or transversion edit within the template. Some MS2 3′-extensions encoded for deletions ranging from 1 nucleotide to 50 nucleotides in length, or for insertions ranging from 1 nucleotide to 39 nucleotides in length, and varied in template lengths. One sgRNA (G034491) targeting the VEGFA locus was also used as a control along with 3 different MS2-loop-linked 3′-extensions each comprising a MS2 loop, a template sequence encoding a GAGG-to-CACT edit in the VEGFA locus, and a DRS targeting VEGFA.

TABLE 12
Exemplary MS2-loop-linked 3′-extensions

| MS2-loop-linked 3′ | MS2-loop-linked 3′ | Target | |
|---|---|---|---|
| extension ID | extension alias | locus | Description/Encoded edit |
| ST034737 (SEQ ID | MS2-only Control | HEK3 | MS2 loop - no template or DRS |
| NO: 601) | | | |
| ST034738 (SEQ ID | WT | HEK3 | 15 nt template, no edit encoded, |
| NO: 602) | | | 10 nt DRS |
| ST034739 (SEQ ID | T > A-positionT1 | HEK3 | 15 nt template, T-to-A edit, 10 nt |
| NO: 603) | | | DRS, Position 1 |
| ST034740 (SEQ ID | T > C-positionT1 | HEK3 | 15 nt template, T-to-C edit, 10 nt |
| NO: 604) | | | DRS, Position 1 |
| ST034741 (SEQ ID | T > G-positionT1 | HEK3 | 15 nt template, T-to-G edit, 10 nt |
| NO: 605) | | | DRS, Position 1 |
| ST034742 (SEQ ID | G > A-positionT2 | HEK3 | 15 nt template, G-to-A edit, 10 nt |
| NO: 606) | | | DRS, Position 2 |
| ST034743 (SEQ ID | G > C-positionT2 | HEK3 | 15 nt template, G-to-C edit, 10 nt |
| NO: 607) | | | DRS, Position 2 |
| ST034744 (SEQ ID | G > T-positionT2 | HEK3 | 15 nt template, G-to-T edit, 10 nt |
| NO: 608) | | | DRS, Position 2 |
| ST034745 (SEQ ID | A > C-positionT3 | HEK3 | 15 nt template, A-to-C edit, 10 nt |
| NO: 609) | | | DRS, Position 3 |
| ST034746 (SEQ ID | A > G-positionT3 | HEK3 | 15 nt template, A-to-G edit, 10 nt |
| NO: 610) | | | DRS, Position 3 |
| ST034748 (SEQ ID | T > A-positionT4 | HEK3 | 15 nt template, T-to-A edit, 10 nt |
| NO: 611) | | | DRS, Position 4 |
| ST034749 (SEQ ID | T > C-positionT4 | HEK3 | 15 nt template, T-to-C edit, 10 nt |
| NO: 612) | | | DRS, Position 4 |
| ST034750 (SEQ ID | T > G-positionT4 | HEK3 | 15 nt template, T-to-G edit, 10 nt |
| NO: 613) | | | DRS, Position 4 |
| ST034751 (SEQ ID | G > C-positionT5 | HEK3 | 15 nt template, G-to-C edit, 10 nt |
| NO: 614) | | | DRS, Position 5 |
| ST034752 (SEQ ID | G > A-positionT5 | HEK3 | 15 nt template, G-to-A edit, 10 nt |
| NO: 615) | | | DRS, Position 5 |
| ST034753 (SEQ ID | G > T-positionT5 | HEK3 | 15 nt template, G-to-T edit, 10 nt |
| NO: 616) | | | DRS, Position 5 |
| ST034754 (SEQ ID | G > C.rNTP-positionT5 | HEK3 | 15 nt template, G-to-C edit, 10 nt |
| NO: 617) | | | DRS, Position 5 |
| ST034755 (SEQ ID | ΔMS2 G > C- | HEK3 | No MS2 loop, 15 nt template, G- |
| NO: 618) | positionT5 | | to-C edit, 10 nt DRS, Position 5 |
| ST034756 (SEQ ID | G > C-positionT6 | HEK3 | 15 nt template, G-to-C edit, 10 nt |
| NO: 619) | | | DRS, Position 6 |
| ST034757 (SEQ ID | C > A-positionT7 | HEK3 | 15 nt template, C-to-A edit, 10 nt |
| NO: 620) | | | DRS, Position 7 |
| ST034758 (SEQ ID | C > G-positionT7 | HEK3 | 15 nt template, C-to-G edit, 10 nt |
| NO: 621) | | | DRS, Position 7 |
| ST034759 (SEQ ID | C > T-positionT7 | HEK3 | 15 nt template, C-to-T edit, 10 nt |
| NO: 622) | | | DRS, Position 7 |
| ST034760 (SEQ ID | A > C-positionT8 | HEK3 | 15 nt template, A-to-C edit, 10 nt |
| NO: 623) | | | DRS, Position 8 |
| ST034761 (SEQ ID | A > G-positionT8 | HEK3 | 15 nt template, A-to-G edit, 10 nt |
| NO: 624) | | | DRS, Position 8 |
| ST034762 (SEQ ID | A > T-positionT8 | HEK3 | 15 nt template, A-to-T edit, 10 nt |
| NO: 625) | | | DRS, Position 8 |
| ST034763 (SEQ ID | G > A-positionT9 | HEK3 | 15 nt template, G-to-A edit, 10 nt |
| NO: 626) | | | DRS, Position 9 |
| ST034765 (SEQ ID | G > T-positionT9 | HEK3 | 15 nt template, G-to-T edit, 10 nt |
| NO: 627) | | | DRS, Position 9 |
| ST034766 (SEQ ID | A > C- positionT10 | HEK3 | 15 nt template, A-to-C edit, 10 nt |
| NO: 628) | | | DRS, Position 10 |
| ST034767 (SEQ ID | A > G-positionT10 | HEK3 | 15 nt template, A-to-G edit, 10 nt |
| NO: 629) | | | DRS, Position 10 |
| ST034768 (SEQ ID | A > T-positionT10 | HEK3 | 15 nt template, A-to-T edit, 10 nt |
| NO: 630) | | | DRS, Position 10 |
| ST034769 (SEQ ID | G > A-positionT11 | HEK3 | 15 nt template, G-to-A edit, 10 nt |
| NO: 631) | | | DRS, Position 11 |
| ST034770 (SEQ ID | G > C-positionT11 | HEK3 | 15 nt template, G-to-C edit, 10 nt |
| NO: 632) | | | DRS, Position 11 |
| ST034771 (SEQ ID | G > T-positionT11 | HEK3 | 15 nt template, G-to-T edit, 10 nt |
| NO: 633) | | | DRS, Position 11 |
| ST034772 (SEQ ID | G > A-positionT12 | HEK3 | 15 nt template, G-to-A edit, 10 nt |
| NO: 634) | | | DRS, Position 12 |
| ST034773 (SEQ ID | G > C-positionT12 | HEK3 | 15 nt template, G-to-C edit, 10 nt |
| NO: 635) | | | DRS, Position 12 |
| ST034774 (SEQ ID | G > T-positionT12 | HEK3 | 15 nt template, G-to-T edit, 10 nt |
| NO: 636) | | | DRS, Position 12 |
| ST034775 (SEQ ID | A > C-positionT13 | HEK3 | 15 nt template, A-to-C edit, 10 nt |
| NO: 637) | | | DRS, Position 13 |
| ST034776 (SEQ ID | A > G-positionT13 | HEK3 | 15 nt template, A-to-G edit, 10 nt |
| NO: 638) | | | DRS, Position 13 |
| ST034777 (SEQ ID | A > T-positionT13 | HEK3 | 15 nt template, A-to-T edit, 10 nt |
| NO: 639) | | | DRS, Position 13 |
| ST034778 (SEQ ID | A > C-positionT14 | HEK3 | 15 nt template, A-to-C edit, 10 nt |
| NO: 640) | | | DRS, Position 14 |
| ST034779 (SEQ ID | A > G-positionT14 | HEK3 | 15 nt template, A-to-G edit, 10 nt |
| NO: 641) | | | DRS, Position 14 |
| ST034780 (SEQ ID | A > T-positionT14 | HEK3 | 15 nt template, A-to-T edit, 10 nt |
| NO: 642) | | | DRS, Position 14 |
| ST034781 (SEQ ID | A > C-positionT15 | HEK3 | 15 nt template, A-to-C edit, 10 nt |
| NO: 643) | | | DRS, Position 15 |
| ST034782 (SEQ ID | A > G-positionT15 | HEK3 | 15 nt template, A-to-G edit, 10 nt |
| NO: 644) | | | DRS, Position 15 |
| ST034783 (SEQ ID | A > T-positionT15 | HEK3 | 15 nt template, A-to-T edit, 10 nt |
| NO: 645) | | | DRS, Position 15 |
| ST034784 (SEQ ID | 15DT-Del1 | HEK3 | 15 nt template, Deletion 1-nt, |
| NO: 646) | | | 10 nt DRS |
| ST034785 (SEQ ID | 20DT-Del1 | HEK3 | 20 nt template, Deletion 1-nt, |
| NO: 647) | | | 10 nt DRS |
| ST034786 (SEQ ID | 30DT-Del1 | HEK3 | 30 nt template, Deletion 1-nt, |
| NO: 648) | | | 10 nt DRS |
| ST034787 (SEQ ID | 50DT-Del1 | HEK3 | 50 nt template, Deletion 1-nt, |
| NO: 649) | | | 10 nt DRS |
| ST034788 (SEQ ID | 15DT-Del5 | HEK3 | 15 nt template, Deletion 5-nt, |
| NO: 650) | | | 10 nt DRS |
| ST034789 (SEQ ID | 20DT-Del5 | HEK3 | 20 nt template, Deletion 5-nt, |
| NO: 651) | | | 10 nt DRS |
| ST034790 (SEQ ID | 30DT-Del5 | HEK3 | 30 nt template, Deletion 5-nt, |
| NO: 652) | | | 10 nt DRS |
| ST034791 (SEQ ID | 50DT-Del5 | HEK3 | 50 nt template, Deletion 5-nt, |
| NO: 653) | | | 10 nt DRS |
| ST034792 (SEQ ID | 15DT-Del10 | HEK3 | 15 nt template, Deletion 10-nt, |
| NO: 654) | | | 10 nt DRS |
| ST034793 (SEQ ID | 20DT-Del10 | HEK3 | 20 nt template, Deletion 10-nt, |
| NO: 655) | | | 10 nt DRS |
| ST034794 (SEQ ID | 30DT-Del10 | HEK3 | 30 nt template, Deletion 10-nt, |
| NO: 656) | | | 10 nt DRS |
| ST034795 (SEQ ID | 50DT-Del10 | HEK3 | 50 nt template, Deletion 10-nt, |
| NO: 657) | | | 10 nt DRS |
| ST034796 (SEQ ID | 15DT-Del25 | HEK3 | 15 nt template, Deletion 25-nt, |
| NO: 658) | | | 10 nt DRS |
| ST034797 (SEQ ID | 20DT-Del25 | HEK3 | 20 nt template, Deletion 25-nt, |
| NO: 659) | | | 10 nt DRS |
| ST034798 (SEQ ID | 30DT-Del25 | HEK3 | 30 nt template, Deletion 25-nt, |
| NO: 660) | | | 10 nt DRS |
| ST034799 (SEQ ID | 50DT-Del25 | HEK3 | 50 nt template, Deletion 25-nt, |
| NO: 661) | | | 10 nt DRS |
| ST034800 (SEQ ID | 15DT-Del50 | HEK3 | 15 nt template, Deletion 50-nt, |
| NO: 662) | | | 10 nt DRS |
| ST034801 (SEQ ID | 20DT-Del50 | HEK3 | 20 nt template, Deletion 50-nt, |
| NO: 663) | | | 10 nt DRS |
| ST034802 (SEQ ID | 30DT-Del50 | HEK3 | 30 nt template, Deletion 50-nt, |
| NO: 664) | | | 10 nt DRS |
| ST034803 (SEQ ID | 50DT-Del50 | HEK3 | 50 nt template, Deletion 50-nt, |
| NO: 665) | | | 10 nt DRS |
| ST034804 (SEQ ID | 15DT-Ins1 | HEK3 | 15 nt template, Insertion 1-nt, |
| NO: 666) | | | 10 nt DRS |
| ST034805 (SEQ ID | 20DT-Ins1 | HEK3 | 20 nt template, Insertion 1-nt, |
| NO: 667) | | | 10 nt DRS |
| ST034806 (SEQ ID | 30DT-Ins1 | HEK3 | 30 nt template, Insertion 1-nt, |
| NO: 668) | | | 10 nt DRS |
| ST034807 (SEQ ID | 50DT-Ins1 | HEK3 | 50 nt template, Insertion 1-nt, |
| NO: 669) | | | 10 nt DRS |
| ST034808 (SEQ ID | 17DT-Ins3 | HEK3 | 17 nt template, Insertion 3-nt, |
| NO: 670) | | | 10 nt DRS |
| ST034809 (SEQ ID | 22DT-Ins3 | HEK3 | 22 nt template, Insertion 3-nt, |
| NO: 671) | | | 10 nt DRS |
| ST034810 (SEQ ID | 32DT-Ins3 | HEK3 | 32 nt template, Insertion 3-nt, |
| NO: 672) | | | 10 nt DRS |
| ST034811 (SEQ ID | 52DT-Ins3 | HEK3 | 52 nt template, Insertion 3-nt, |
| NO: 673) | | | 10 nt DRS |
| ST034812 (SEQ ID | 23DT-Ins9 | HEK3 | 23 nt template, Insertion 9-nt, |
| NO: 674) | | | 10 nt DRS |
| ST034813 (SEQ ID | 26DT-Ins9 | HEK3 | 26 nt template, Insertion 9-nt, |
| NO: 675) | | | 10 nt DRS |
| ST034814 (SEQ ID | 36DT-Ins9 | HEK3 | 36 nt template, Insertion 9-nt, |
| NO: 676) | | | 10 nt DRS |
| ST034815 (SEQ ID | 56DT-Ins9 | HEK3 | 56 nt template, Insertion 9-nt, |
| NO: 677) | | | 10 nt DRS |
| ST034816 (SEQ ID | 35DT-Ins21 | HEK3 | 35 nt template, Insertion 21-nt, |
| NO: 678) | | | 10 nt DRS |
| ST034817 (SEQ ID | 40DT-Ins21 | HEK3 | 40 nt template, Insertion 21-nt, |
| NO: 679) | | | 10 nt DRS |
| ST034818 (SEQ ID | 50DT-Ins21 | HEK3 | 50 nt template, Insertion 21-nt, |
| NO: 680) | | | 10 nt DRS |
| ST034819 (SEQ ID | 70DT-Ins21 | HEK3 | 70 nt template, Insertion 21-nt, |
| NO: 681) | | | 10 nt DRS |
| ST034824 (SEQ ID | 53DT-Ins39 | HEK3 | 53 nt template, Insertion 39-nt, |
| NO: 682) | | | 10 nt DRS |
| ST034825 (SEQ ID | 58DT-Ins39 | HEK3 | 58 nt template, Insertion 39-nt, |
| NO: 683) | | | 10 nt DRS |
| ST034826 (SEQ ID | 68DT-Ins39 | HEK3 | 68 nt template, Insertion 39-nt, |
| NO: 684) | | | 10 nt DRS |
| ST034827 (SEQ ID | 77DT-Ins39 | HEK3 | 77 nt template, Insertion 39-nt, |
| NO: 685) | | | 10 nt DRS |
| ST034641 (SEQ ID | 18DT-17DRS | VEGFA | 18 nt template, GAGG-to-CACT |
| NO: 686) | | | edit, 17-nt DRS |
| ST034828 (SEQ ID | 18RT-17DRS | VEGFA | 18 nt template, GAGG-to-CACT |
| NO: 687) | | | edit, 17-nt DRS |
| ST034829 (SEQ ID | 18DT-11DRS | VEGFA | 18 nt template, GAGG-to-CACT |
| NO: 688) | | | edit, 11-nt DRS |

MS2-loop-linked 3′-extensions were delivered into human hepatoma (Huh7) cells along with a sgRNA targeting the HEK3 or VEGFA locus to assess efficiency of various types of templated edits including transitions and transversions at multiple locations within the target DNA, as well as insertions and deletions of various lengths.

Huh7 cells were prepared as described in Example 1, plated at a density of 15,000 cells/well, and incubated in a tissue culture incubator at 37° C. and 5% CO₂ atmosphere for 24 hours. After incubation, sgRNAs and MS2-loop-linked 3′ extensions were heat shocked at 95 C for 3 minutes and immediately placed on ice for 5 minutes before dilution in Opti-MEM™ (Gibco, Cat. 31985-062). Cells were transfected using Lipofectamine™ MessengerMAX™ Reagent (Invitrogen, Cat. LMRNA015) according to manufacturer's protocol, with 25 nM of MS2-loop-linked 3′-extension and 25 nM of sgRNA in a first lipoplex, and 100 ng of mRNA (encoding a SpyCas9 (SEQ ID NO: 1001) or a nCas9-polymerase (SEQ ID NO: 1010) fusion protein) in a second lipoplex, in a total of 0.3 μL lipoplex. Samples were run in duplicates. The cells were lysed 72 hours post-treatment for NGS analysis as described in Example 1.

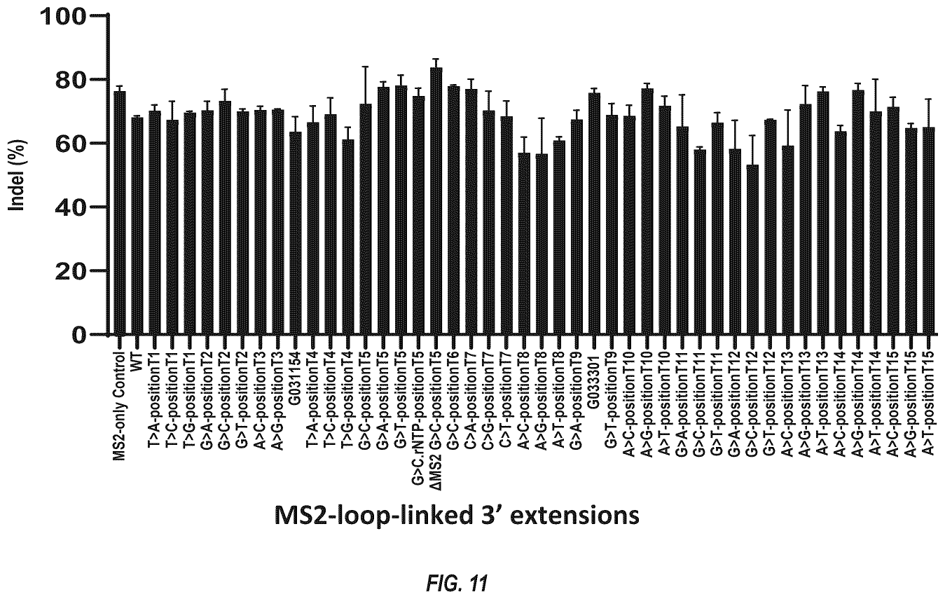

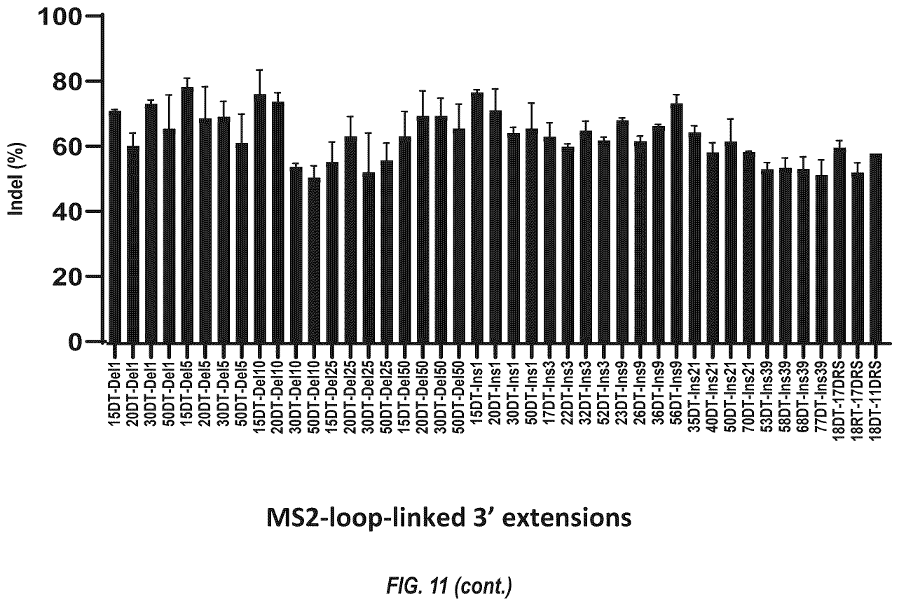

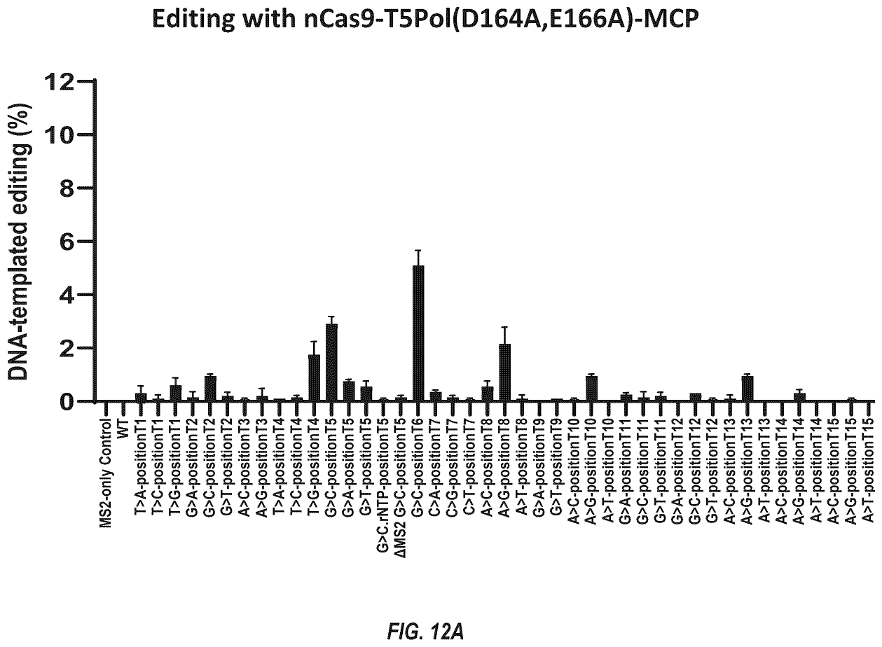

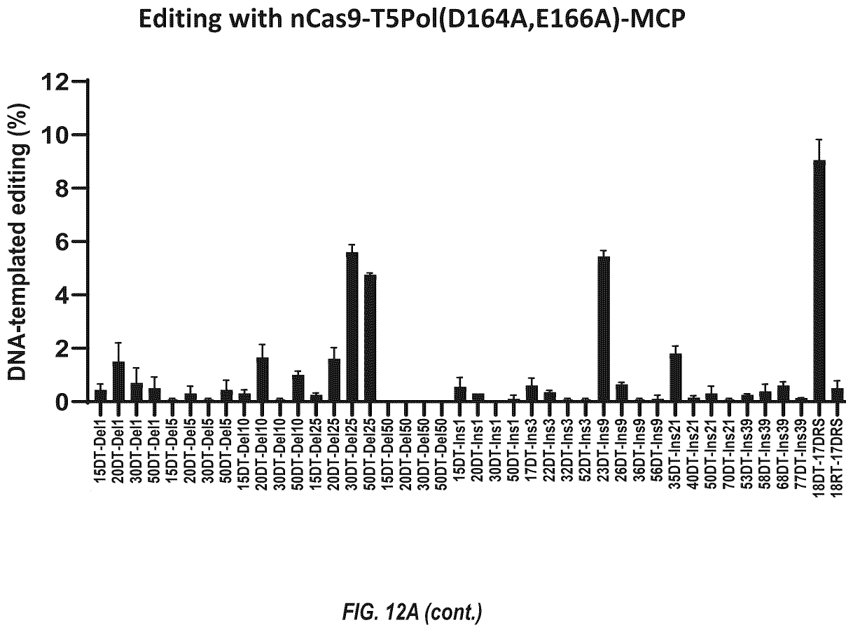

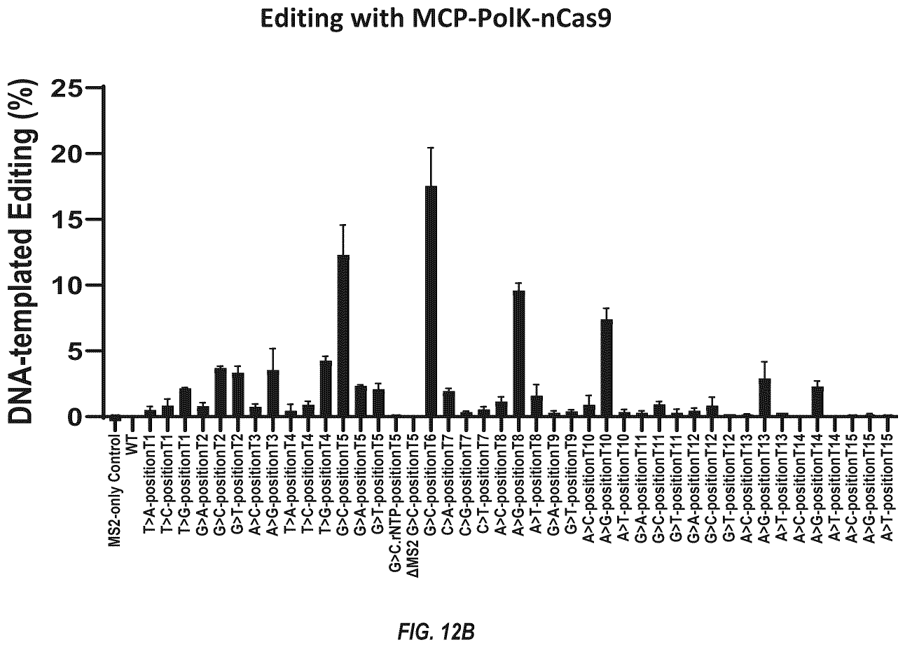

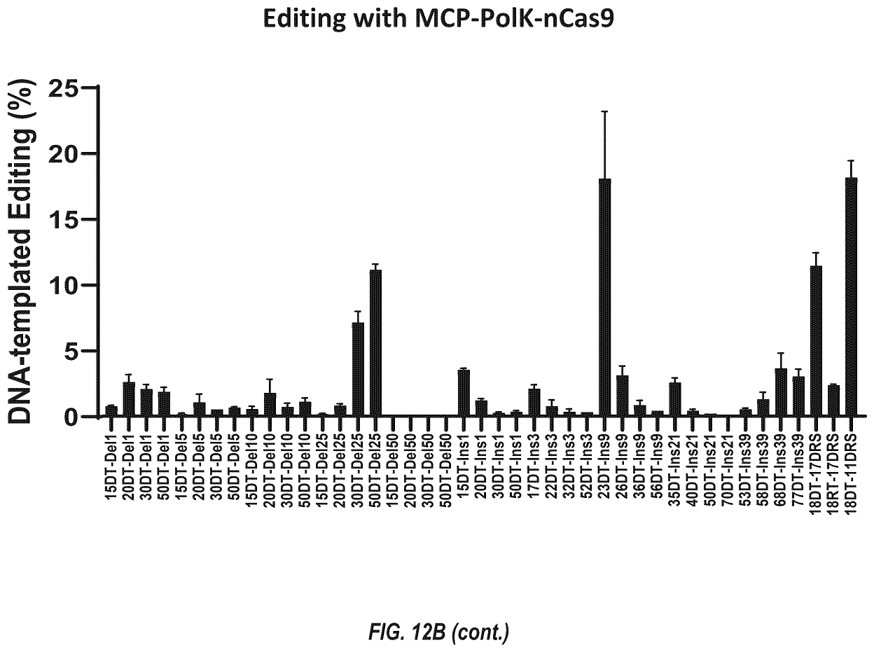

Mean indel results with standard deviation (SD) are shown in FIG. 11 and Table 13. Mean percent templated edit results with standard deviation (SD) are shown in FIGS. 12A-B and Table 14.

TABLE 13
Mean percent indels at HEK3 or VEGFA locus
in Huh7 cell using SpyCas9 mRNA.

| | | Mean | |
|---|---|---|---|
| MS2-loop-linked 3′ | MS2-loop-linked 3′ | Indel | |
| extension ID | extension alias | (%) | SD |
| ST034737 | MS2-only Control | 76.40 | 1.56 |
| ST034738 | WT | 68.15 | 0.49 |
| ST034739 | T > A-positionT1 | 70.20 | 1.84 |
| ST034740 | T > C-positionT1 | 67.35 | 5.87 |
| ST034741 | T > G-positionT1 | 69.60 | 0.42 |
| ST034742 | G > A-positionT2 | 70.35 | 2.90 |
| ST034743 | G > C-positionT2 | 73.30 | 3.68 |
| ST034744 | G > T-positionT2 | 70.05 | 0.78 |
| ST034745 | A > C-positionT3 | 70.45 | 1.20 |
| ST034746 | A > G-positionT3 | 70.50 | 0.28 |
| G031154 (tgRNA benchmark) | G031154 | 63.65 | 4.74 |
| (SEQ ID NO: 480) | | | |
| ST034748 | T > A-positionT4 | 66.65 | 5.02 |
| ST034749 | T > C-positionT4 | 69.15 | 5.16 |
| ST034750 | T > G-positionT4 | 61.20 | 3.82 |
| ST034751 | G > C-positionT5 | 72.35 | 11.67 |
| ST034752 | G > A-positionT5 | 77.70 | 1.56 |
| ST034753 | G > T-positionT5 | 78.10 | 3.25 |
| ST034754 | G > C.rNTP-positionT5 | 74.85 | 2.47 |
| ST034755 | ΔMS2 G > C-positionT5 | 83.80 | 2.69 |
| ST034756 | G > C-positionT6 | 77.95 | 0.35 |
| ST034757 | C > A-positionT7 | 77.05 | 3.04 |
| ST034758 | C > G-positionT7 | 70.25 | 6.15 |
| ST034759 | C > T-positionT7 | 68.45 | 4.88 |
| ST034760 | A > C-positionT8 | 57.00 | 4.95 |
| ST034761 | A > G-positionT8 | 56.70 | 11.17 |
| ST034762 | A > T-positionT8 | 60.90 | 1.13 |
| ST034763 | G > A-positionT9 | 67.45 | 2.90 |
| G033301 (sgRNA benchmark) | G033301 | 75.75 | 1.48 |
| ST034765 | G > T-positionT9 | 68.85 | 3.61 |
| ST034766 | A > C-positionT10 | 68.65 | 3.32 |
| ST034767 | A > G-positionT10 | 77.20 | 1.56 |
| ST034768 | A > T-positionT10 | 71.75 | 3.04 |
| ST034769 | G > A-positionT11 | 65.30 | 9.90 |
| ST034770 | G > C-positionT11 | 58.00 | 0.85 |
| ST034771 | G > T-positionT11 | 66.45 | 3.18 |
| ST034772 | G > A-positionT12 | 58.30 | 8.91 |
| ST034773 | G > C-positionT12 | 53.30 | 9.19 |
| ST034774 | G > T-positionT12 | 67.35 | 0.21 |
| ST034775 | A > C-positionT13 | 59.25 | 11.24 |
| ST034776 | A > G-positionT13 | 72.25 | 5.87 |
| ST034777 | A > T-positionT13 | 76.30 | 1.41 |
| ST034778 | A > C-positionT14 | 63.75 | 1.91 |
| ST034779 | A > G-positionT14 | 76.70 | 2.12 |
| ST034780 | A > T-positionT14 | 70.05 | 10.11 |
| ST034781 | A > C-positionT15 | 71.45 | 3.04 |
| ST034782 | A > G-positionT15 | 64.80 | 1.41 |
| ST034783 | A > T-positionT15 | 65.05 | 8.84 |
| ST034784 | 15DT-Del1 | 71.70 | 0.42 |
| ST034785 | 20DT-Del1 | 60.95 | 3.89 |
| ST034786 | 30DT-Del1 | 73.85 | 1.20 |
| ST034787 | 50DT-Del1 | 66.20 | 10.47 |
| ST034788 | 15DT-Del5 | 79.15 | 2.62 |
| ST034789 | 20DT-Del5 | 69.40 | 9.76 |
| ST034790 | 30DT-Del5 | 69.85 | 4.74 |
| ST034791 | 50DT-Del5 | 61.80 | 8.91 |
| ST034792 | 15DT-Del10 | 76.85 | 7.42 |
| ST034793 | 20DT-Del10 | 74.50 | 2.83 |
| ST034794 | 30DT-Del10 | 54.45 | 1.06 |
| ST034795 | 50DT-Del10 | 51.10 | 3.68 |
| ST034796 | 15DT-Del25 | 55.95 | 6.15 |
| ST034797 | 20DT-Del25 | 63.90 | 6.08 |
| ST034798 | 30DT-Del25 | 52.75 | 12.09 |
| ST034799 | 50DT-Del25 | 56.45 | 5.30 |
| ST034800 | 15DT-Del50 | 63.90 | 7.64 |
| ST034801 | 20DT-Del50 | 70.10 | 7.78 |
| ST034802 | 30DT-Del50 | 70.10 | 5.52 |
| ST034803 | 50DT-Del50 | 66.25 | 7.57 |
| ST034804 | 15DT-Ins1 | 77.40 | 0.85 |
| ST034805 | 20DT-Ins1 | 71.85 | 6.58 |
| ST034806 | 30DT-Ins1 | 64.85 | 1.77 |
| ST034807 | 50DT-Ins1 | 66.25 | 7.85 |
| ST034808 | 17DT-Ins3 | 63.75 | 4.31 |
| ST034809 | 22DT-Ins3 | 60.65 | 0.92 |
| ST034810 | 32DT-Ins3 | 65.60 | 2.97 |
| ST034811 | 52DT-Ins3 | 62.60 | 0.99 |
| ST034812 | 23DT-Ins9 | 68.80 | 0.71 |
| ST034813 | 26DT-Ins9 | 62.40 | 1.56 |
| ST034814 | 36DT-Ins9 | 67.05 | 0.49 |
| ST034815 | 56DT-Ins9 | 74.00 | 2.69 |
| ST034816 | 35DT-Ins21 | 65.05 | 2.05 |
| ST034817 | 40DT-Ins21 | 58.85 | 3.04 |
| ST034818 | 50DT-Ins21 | 62.30 | 6.93 |
| ST034819 | 70DT-Ins21 | 59.05 | 0.21 |
| ST034824 | 53DT-Ins39 | 53.70 | 2.12 |
| ST034825 | 58DT-Ins39 | 54.15 | 3.04 |
| ST034826 | 68DT-Ins39 | 53.90 | 3.68 |
| ST034827 | 77DT-Ins39 | 51.85 | 4.74 |
| ST034641 | 18DT-17DRS | 60.40 | 2.12 |
| ST034828 | 18RT-17DRS | 52.70 | 2.97 |
| ST034829 | 18DT-11DRS | 58.50 | 0.00 |

TABLE 14
Mean percent templated editing at the HEK3 or VEGFA locus in Huh7
cells using template based editing with nCas9-T5Pol(D164A, E166A)-
MCP (SEQ ID NO: 1168), or MCP-PolK-nCas9 (SEQ ID NO: 1057)

| | | | Mean | |
|---|---|---|---|---|
| | | | DNA- | |
| MS2-loop- | MS2-loop- | | templated | |
| linked 3′ | linked 3′ | | Editing | |
| extension ID | extension alias | mRNA ID | (%) | SD |
| ST034737 | MS2-only | nCas9-T5Pol(D164A, E166A)-MCP | 0.00 | 0.00 |
| | Control | | | |
| ST034738 | WT | nCas9-T5Pol(D164A, E166A)-MCP | 0.00 | 0.00 |
| ST034739 | T > A-positionT1 | nCas9-T5Pol(D164A, E166A)-MCP | 0.30 | 0.28 |
| ST034740 | T > C-positionT1 | nCas9-T5Pol(D164A, E166A)-MCP | 0.10 | 0.14 |
| ST034741 | T > G-positionT1 | nCas9-T5Pol(D164A, E166A)-MCP | 0.60 | 0.28 |
| ST034742 | G > A-positionT2 | nCas9-T5Pol(D164A, E166A)-MCP | 0.15 | 0.21 |
| ST034743 | G > C-positionT2 | nCas9-T5Pol(D164A, E166A)-MCP | 0.95 | 0.07 |
| ST034744 | G > T-positionT2 | nCas9-T5Pol(D164A, E166A)-MCP | 0.20 | 0.14 |
| ST034745 | A > C-positionT3 | nCas9-T5Pol(D164A, E166A)-MCP | 0.05 | 0.07 |
| ST034746 | A > G-positionT3 | nCas9-T5Pol(D164A, E166A)-MCP | 0.20 | 0.28 |
| ST034748 | T > A-positionT4 | nCas9-T5Pol(D164A, E166A)-MCP | 0.10 | 0.00 |
| ST034749 | T > C-positionT4 | nCas9-T5Pol(D164A, E166A)-MCP | 0.15 | 0.07 |
| ST034750 | T > G-positionT4 | nCas9-T5Pol(D164A, E166A)-MCP | 1.75 | 0.49 |
| ST034751 | G > C-positionT5 | nCas9-T5Pol(D164A, E166A)-MCP | 2.90 | 0.28 |
| ST034752 | G > A-positionT5 | nCas9-T5Pol(D164A, E166A)-MCP | 0.75 | 0.07 |
| ST034753 | G > T-positionT5 | nCas9-T5Pol(D164A, E166A)-MCP | 0.55 | 0.21 |
| ST034754 | G > C.rNTP- | nCas9-T5Pol(D164A, E166A)-MCP | 0.05 | 0.07 |
| | positionT5 | | | |
| ST034755 | ΔMS2 G > C- | nCas9-T5Pol(D164A, E166A)-MCP | 0.15 | 0.07 |
| | positionT5 | | | |
| ST034756 | G > C-positionT6 | nCas9-T5Pol(D164A, E166A)-MCP | 5.10 | 0.57 |
| ST034757 | C > A-positionT7 | nCas9-T5Pol(D164A, E166A)-MCP | 0.35 | 0.07 |
| ST034758 | C > G-positionT7 | nCas9-T5Pol(D164A, E166A)-MCP | 0.15 | 0.07 |
| ST034759 | C > T-positionT7 | nCas9-T5Pol(D164A, E166A)-MCP | 0.05 | 0.07 |
| ST034760 | A > C-positionT8 | nCas9-T5Pol(D164A, E166A)-MCP | 0.55 | 0.21 |
| ST034761 | A > G-positionT8 | nCas9-T5Pol(D164A, E166A)-MCP | 2.15 | 0.64 |
| ST034762 | A > T-positionT8 | nCas9-T5Pol(D164A, E166A)-MCP | 0.10 | 0.14 |
| ST034763 | G > A-positionT9 | nCas9-T5Pol(D164A, E166A)-MCP | 0.00 | 0.00 |
| ST034765 | G > T-positionT9 | nCas9-T5Pol(D164A, E166A)-MCP | 0.10 | 0.00 |
| ST034766 | A > C- | nCas9-T5Pol(D164A, E166A)-MCP | 0.05 | 0.07 |
| | positionT1positionT10 | | | |
| ST034767 | A > G- | nCas9-T5Pol(D164A, E166A)-MCP | 0.95 | 0.07 |
| | positionT1positionT10 | | | |
| ST034768 | A > T- | nCas9-T5Pol(D164A, E166A)-MCP | 0.00 | 0.00 |
| | positionT1positionT10 | | | |
| ST034769 | G > A- | nCas9-T5Pol(D164A, E166A)-MCP | 0.25 | 0.07 |
| | positionT1positionT11 | | | |
| ST034770 | G > C- | nCas9-T5Pol(D164A, E166A)-MCP | 0.15 | 0.21 |
| | positionT1positionT11 | | | |
| ST034771 | G > T- | nCas9-T5Pol(D164A, E166A)-MCP | 0.20 | 0.14 |
| | positionT1positionT11 | | | |
| ST034772 | G > A- | nCas9-T5Pol(D164A, E166A)-MCP | 0.00 | 0.00 |
| | positionT1positionT12 | | | |
| ST034773 | G > C- | nCas9-T5Pol(D164A, E166A)-MCP | 0.30 | 0.00 |
| | positionT1positionT12 | | | |
| ST034774 | G > T- | nCas9-T5Pol(D164A, E166A)-MCP | 0.05 | 0.07 |
| | positionT1positionT12 | | | |
| ST034775 | A > C- | nCas9-T5Pol(D164A, E166A)-MCP | 0.10 | 0.14 |
| | positionT1positionT13 | | | |
| ST034776 | A > G- | nCas9-T5Pol(D164A, E166A)-MCP | 0.95 | 0.07 |
| | positionT1positionT13 | | | |
| ST034777 | A > T- | nCas9-T5Pol(D164A, E166A)-MCP | 0.00 | 0.00 |
| | positionT1positionT13 | | | |
| ST034778 | A > C- | nCas9-T5Pol(D164A, E166A)-MCP | 0.00 | 0.00 |
| | positionT1positionT14 | | | |
| ST034779 | A > G- | nCas9-T5Pol(D164A, E166A)-MCP | 0.30 | 0.14 |
| | positionT1positionT14 | | | |
| ST034780 | A > T- | nCas9-T5Pol(D164A, E166A)-MCP | 0.00 | 0.00 |
| | positionT1positionT14 | | | |
| ST034781 | A > C- | nCas9-T5Pol(D164A, E166A)-MCP | 0.00 | 0.00 |
| | positionT1positionT15 | | | |
| ST034782 | A > G- | nCas9-T5Pol(D164A, E166A)-MCP | 0.05 | 0.07 |
| | positionT1positionT15 | | | |
| ST034783 | A > T- | nCas9-T5Pol(D164A, E166A)-MCP | 0.00 | 0.00 |
| | positionT1positionT15 | | | |
| ST034784 | 15DT-Del1 | nCas9-T5Pol(D164A, E166A)-MCP | 0.45 | 0.21 |
| ST034785 | 20DT-Del1 | nCas9-T5Pol(D164A, E166A)-MCP | 1.50 | 0.71 |
| ST034786 | 30DT-Del1 | nCas9-T5Pol(D164A, E166A)-MCP | 0.70 | 0.57 |
| ST034787 | 50DT-Del1 | nCas9-T5Pol(D164A, E166A)-MCP | 0.50 | 0.42 |
| ST034788 | 15DT-Del5 | nCas9-T5Pol(D164A, E166A)-MCP | 0.05 | 0.07 |
| ST034789 | 20DT-Del5 | nCas9-T5Pol(D164A, E166A)-MCP | 0.30 | 0.28 |
| ST034790 | 30DT-Del5 | nCas9-T5Pol(D164A, E166A)-MCP | 0.05 | 0.07 |
| ST034791 | 50DT-Del5 | nCas9-T5Pol(D164A, E166A)-MCP | 0.45 | 0.35 |
| ST034792 | 15DT-Del10 | nCas9-T5Pol(D164A, E166A)-MCP | 0.30 | 0.14 |
| ST034793 | 20DT-Del10 | nCas9-T5Pol(D164A, E166A)-MCP | 1.65 | 0.49 |
| ST034794 | 30DT-Del10 | nCas9-T5Pol(D164A, E166A)-MCP | 0.05 | 0.07 |
| ST034795 | 50DT-Del10 | nCas9-T5Pol(D164A, E166A)-MCP | 1.00 | 0.14 |
| ST034796 | 15DT-Del25 | nCas9-T5Pol(D164A, E166A)-MCP | 0.25 | 0.07 |
| ST034797 | 20DT-Del25 | nCas9-T5Pol(D164A, E166A)-MCP | 1.60 | 0.42 |
| ST034798 | 30DT-Del25 | nCas9-T5Pol(D164A, E166A)-MCP | 5.60 | 0.28 |
| ST034799 | 50DT-Del25 | nCas9-T5Pol(D164A, E166A)-MCP | 4.75 | 0.07 |
| ST034800 | 15DT-Del50 | nCas9-T5Pol(D164A, E166A)-MCP | Technical | Technical |
| | | | Fail | Fail |
| ST034801 | 20DT-Del50 | nCas9-T5Pol(D164A, E166A)-MCP | Technical | Technical |
| | | | Fail | Fail |
| ST034802 | 30DT-Del50 | nCas9-T5Pol(D164A, E166A)-MCP | Technical | Technical |
| | | | Fail | Fail |
| ST034803 | 50DT-De150 | nCas9-T5Pol(D164A, E166A)-MCP | Technical | Technical |
| | | | Fail | Fail |
| ST034804 | 15DT-Ins1 | nCas9-T5Pol(D164A, E166A)-MCP | 0.55 | 0.35 |
| ST034805 | 20DT-Ins1 | nCas9-T5Pol(D164A, E166A)-MCP | 0.30 | 0.00 |
| ST034806 | 30DT-Ins1 | nCas9-T5Pol(D164A, E166A)-MCP | 0.00 | 0.00 |
| ST034807 | 50DT-Ins1 | nCas9-T5Pol(D164A, E166A)-MCP | 0.10 | 0.14 |
| ST034808 | 17DT-Ins3 | nCas9-T5Pol(D164A, E166A)-MCP | 0.60 | 0.28 |
| ST034809 | 22DT-Ins3 | nCas9-T5Pol(D164A, E166A)-MCP | 0.35 | 0.07 |
| ST034810 | 32DT-Ins3 | nCas9-T5Pol(D164A, E166A)-MCP | 0.05 | 0.07 |
| ST034811 | 52DT-Ins3 | nCas9-T5Pol(D164A, E166A)-MCP | 0.05 | 0.07 |
| ST034812 | 23DT-Ins9 | nCas9-T5Pol(D164A, E166A)-MCP | 5.45 | 0.21 |
| ST034813 | 26DT-Ins9 | nCas9-T5Pol(D164A, E166A)-MCP | 0.65 | 0.07 |
| ST034814 | 36DT-Ins9 | nCas9-T5Pol(D164A, E166A)-MCP | 0.05 | 0.07 |
| ST034815 | 56DT-Ins9 | nCas9-T5Pol(D164A, E166A)-MCP | 0.10 | 0.14 |
| ST034816 | 35DT-Ins21 | nCas9-T5Pol(D164A, E166A)-MCP | 1.80 | 0.28 |
| ST034817 | 40DT-Ins21 | nCas9-T5Pol(D164A, E166A)-MCP | 0.15 | 0.07 |
| ST034818 | 50DT-Ins21 | nCas9-T5Pol(D164A, E166A)-MCP | 0.30 | 0.28 |
| ST034819 | 70DT-Ins21 | nCas9-T5Pol(D164A, E166A)-MCP | 0.05 | 0.07 |
| ST034824 | 53DT-Ins39 | nCas9-T5Pol(D164A, E166A)-MCP | 0.26 | 0.03 |
| ST034825 | 58DT-Ins39 | nCas9-T5Pol(D164A, E166A)-MCP | 0.38 | 0.27 |
| ST034826 | 68DT-Ins39 | nCas9-T5Pol(D164A, E166A)-MCP | 0.61 | 0.14 |
| ST034827 | 77DT-Ins39 | nCas9-T5Pol(D164A, E166A)-MCP | 0.14 | 0.01 |
| ST034641 | 18DT-17DRS | nCas9-T5Pol(D164A, E166A)-MCP | 9.05 | 0.78 |
| ST034828 | 18RT-17DRS | nCas9-T5Pol(D164A, E166A)-MCP | 0.50 | 0.28 |
| ST034737 | MS2-only | MCP-PolK-nCas9 | 0.05 | 0.07 |
| | Control | | | |
| ST034738 | WT | MCP-PolK-nCas9 | 0.00 | 0.00 |
| ST034739 | T > A-positionT1 | MCP-PolK-nCas9 | 0.50 | 0.28 |
| ST034740 | T > C-positionT1 | MCP-PolK-nCas9 | 0.85 | 0.49 |
| ST034741 | T > G-positionT1 | MCP-PolK-nCas9 | 2.15 | 0.07 |
| ST034742 | G > A-positionT2 | MCP-PolK-nCas9 | 0.80 | 0.28 |
| ST034743 | G > C-positionT2 | MCP-PolK-nCas9 | 3.70 | 0.14 |
| ST034744 | G > T-positionT2 | MCP-PolK-nCas9 | 3.35 | 0.49 |
| ST034745 | A > C-positionT3 | MCP-PolK-nCas9 | 0.75 | 0.21 |
| ST034746 | A > G-positionT3 | MCP-PolK-nCas9 | 3.55 | 1.63 |
| ST034748 | T > A-positionT4 | MCP-PolK-nCas9 | 0.45 | 0.49 |
| ST034749 | T > C-positionT4 | MCP-PolK-nCas9 | 0.90 | 0.28 |
| ST034750 | T > G-positionT4 | MCP-PolK-nCas9 | 4.25 | 0.35 |
| ST034751 | G > C-positionT5 | MCP-PolK-nCas9 | 12.30 | 2.26 |
| ST034752 | G > A-positionT5 | MCP-PolK-nCas9 | 2.35 | 0.07 |
| ST034753 | G > T-positionT5 | MCP-PolK-nCas9 | 2.10 | 0.42 |
| ST034754 | G > C.rNTP- | MCP-PolK-nCas9 | 0.05 | 0.07 |
| | positionT5 | | | |
| ST034755 | ΔMS2 G > C- | MCP-PolK-nCas9 | 0.00 | 0.00 |
| | positionT5 | | | |
| ST034756 | G > C-positionT6 | MCP-PolK-nCas9 | 17.55 | 2.90 |
| ST034757 | C > A-positionT7 | MCP-PolK-nCas9 | 1.95 | 0.21 |
| ST034758 | C > G-positionT7 | MCP-PolK-nCas9 | 0.35 | 0.07 |
| ST034759 | C > T-positionT7 | MCP-PolK-nCas9 | 0.55 | 0.21 |
| ST034760 | A > C-positionT8 | MCP-PolK-nCas9 | 1.15 | 0.35 |
| ST034761 | A > G-positionT8 | MCP-PolK-nCas9 | 9.60 | 0.57 |
| ST034762 | A > T-positionT8 | MCP-PolK-nCas9 | 1.60 | 0.85 |
| ST034763 | G > A-positionT9 | MCP-PolK-nCas9 | 0.30 | 0.14 |
| ST034765 | G > T-positionT9 | MCP-PolK-nCas9 | 0.40 | 0.14 |
| ST034766 | A > C- | MCP-PolK-nCas9 | 0.90 | 0.71 |
| | positionT1positionT10 | | | |
| ST034767 | A > G- | MCP-PolK-nCas9 | 7.40 | 0.85 |
| | positionT1positionT10 | | | |
| ST034768 | A > T- | MCP-PolK-nCas9 | 0.35 | 0.21 |
| | positionT1positionT10 | | | |
| ST034769 | G > A- | MCP-PolK-nCas9 | 0.30 | 0.14 |
| | positionT1positionT11 | | | |
| ST034770 | G > C- | MCP-PolK-nCas9 | 0.95 | 0.21 |
| | positionT1positionT11 | | | |
| ST034771 | G > T- | MCP-PolK-nCas9 | 0.30 | 0.28 |
| | positionT1positionT11 | | | |
| ST034772 | G > A- | MCP-PolK-nCas9 | 0.45 | 0.21 |
| | positionT1positionT12 | | | |
| ST034773 | G > C- | MCP-PolK-nCas9 | 0.85 | 0.64 |
| | positionT1positionT12 | | | |
| ST034774 | G > T- | MCP-PolK-nCas9 | 0.20 | 0.00 |
| | positionT1positionT12 | | | |
| ST034775 | A > C- | MCP-PolK-nCas9 | 0.15 | 0.07 |
| | positionT1positionT13 | | | |
| ST034776 | A > G- | MCP-PolK-nCas9 | 2.90 | 1.27 |
| | positionT1positionT13 | | | |
| ST034777 | A > T- | MCP-PolK-nCas9 | 0.30 | 0.00 |
| | positionT1positionT13 | | | |
| ST034778 | A > C- | MCP-PolK-nCas9 | 0.00 | 0.00 |
| | positionT1positionT14 | | | |
| ST034779 | A > G- | MCP-PolK-nCas9 | 2.30 | 0.42 |
| | positionT1positionT14 | | | |
| ST034780 | A > T- | MCP-PolK-nCas9 | 0.00 | 0.00 |
| | positionT1positionT14 | | | |
| ST034781 | A > C- | MCP-PolK-nCas9 | 0.05 | 0.07 |
| | positionT1positionT15 | | | |
| ST034782 | A > G- | MCP-PolK-nCas9 | 0.10 | 0.14 |
| | positionT1positionT15 | | | |
| ST034783 | A > T- | MCP-PolK-nCas9 | 0.05 | 0.07 |
| | positionT1positionT15 | | | |
| ST034784 | 15DT-Del1 | MCP-PolK-nCas9 | 0.75 | 0.07 |
| ST034785 | 20DT-Del1 | MCP-PolK-nCas9 | 2.60 | 0.57 |
| ST034786 | 30DT-Del1 | MCP-PolK-nCas9 | 2.05 | 0.35 |
| ST034787 | 50DT-Del1 | MCP-PolK-nCas9 | 1.85 | 0.35 |
| ST034788 | 15DT-Del5 | MCP-PolK-nCas9 | 0.10 | 0.14 |
| ST034789 | 20DT-Del5 | MCP-PolK-nCas9 | 1.05 | 0.64 |
| ST034790 | 30DT-Del5 | MCP-PolK-nCas9 | 0.50 | 0.00 |
| ST034791 | 50DT-Del5 | MCP-PolK-nCas9 | 0.65 | 0.07 |
| ST034792 | 15DT-Del10 | MCP-PolK-nCas9 | 0.55 | 0.21 |
| ST034793 | 20DT-Del10 | MCP-PolK-nCas9 | 1.75 | 1.06 |
| ST034794 | 30DT-Del10 | MCP-PolK-nCas9 | 0.70 | 0.28 |
| ST034795 | 50DT-Del10 | MCP-PolK-nCas9 | 1.10 | 0.28 |
| ST034796 | 15DT-Del25 | MCP-PolK-nCas9 | 0.15 | 0.07 |
| ST034797 | 20DT-Del25 | MCP-PolK-nCas9 | 0.80 | 0.14 |
| ST034798 | 30DT-Del25 | MCP-PolK-nCas9 | 7.10 | 0.85 |
| ST034799 | 50DT-Del25 | MCP-PolK-nCas9 | 11.10 | 0.42 |
| ST034800 | 15DT-Del50 | MCP-PolK-nCas9 | Technical | Technical |
| | | | Fail | Fail |
| ST034801 | 20DT-Del50 | MCP-PolK-nCas9 | Technical | Technical |
| | | | Fail | Fail |
| ST034802 | 30DT-Del50 | MCP-PolK-nCas9 | Technical | Technical |
| | | | Fail | Fail |
| ST034803 | 50DT-Del50 | MCP-PolK-nCas9 | Technical | Technical |
| | | | Fail | Fail |
| ST034804 | 15DT-Ins1 | MCP-PolK-nCas9 | 3.50 | 0.14 |
| ST034805 | 20DT-Ins1 | MCP-PolK-nCas9 | 1.20 | 0.14 |
| ST034806 | 30DT-Ins1 | MCP-PolK-nCas9 | 0.25 | 0.07 |
| ST034807 | 50DT-Ins1 | MCP-PolK-nCas9 | 0.35 | 0.07 |
| ST034808 | 17DT-Ins3 | MCP-PolK-nCas9 | 2.10 | 0.28 |
| ST034809 | 22DT-Ins3 | MCP-PolK-nCas9 | 0.75 | 0.49 |
| ST034810 | 32DT-Ins3 | MCP-PolK-nCas9 | 0.35 | 0.21 |
| ST034811 | 52DT-Ins3 | MCP-PolK-nCas9 | 0.30 | 0.00 |
| ST034812 | 23DT-Ins9 | MCP-PolK-nCas9 | 18.00 | 5.09 |
| ST034813 | 26DT-Ins9 | MCP-PolK-nCas9 | 3.10 | 0.71 |
| ST034814 | 36DT-Ins9 | MCP-PolK-nCas9 | 0.85 | 0.35 |
| ST034815 | 56DT-Ins9 | MCP-PolK-nCas9 | 0.40 | 0.00 |
| ST034816 | 35DT-Ins21 | MCP-PolK-nCas9 | 2.55 | 0.35 |
| ST034817 | 40DT-Ins21 | MCP-PolK-nCas9 | 0.40 | 0.14 |
| ST034818 | 50DT-Ins21 | MCP-PolK-nCas9 | 0.20 | 0.00 |
| ST034819 | 70DT-Ins21 | MCP-PolK-nCas9 | 0.00 | 0.00 |
| ST034824 | 53DT-Ins39 | MCP-PolK-nCas9 | 0.54 | 0.08 |
| ST034825 | 58DT-Ins39 | MCP-PolK-nCas9 | 1.29 | 0.54 |
| ST034826 | 68DT-Ins39 | MCP-PolK-nCas9 | 3.63 | 1.16 |
| ST034827 | 77DT-Ins39 | MCP-PolK-nCas9 | 3.01 | 0.57 |
| ST034641 | 18DT-17DRS | MCP-PolK-nCas9 | 11.40 | 0.99 |
| ST034828 | 18RT-17DRS | MCP-PolK-nCas9 | 2.35 | 0.07 |
| ST034829 | 18DT-11DRS | MCP-PolK-nCas9 | 18.10 | 1.27 |

A second experiment was conducted using the same guides and MS2-loop-linked 3′ extensions as listed in Table 12, following the same protocol as described above. Huh7 cells were transfected with 25 nM of MS2-loop-linked 3′ extension and 25 nM of sgRNA in a first lipoplex, and 100 ng of mRNA encoding MCP-PolK-nCas9 in a second lipoplex, in a total of 0.3 μL lipoplex. When used, ngRNA was delivered in the same guide RNA lipoplex at a 5:3:2 sgRNA: MS2-loop-linked 3′-extension: ngRNA ratio (25 nM sgRNA, 15 nM MS2-loop-linked 3′ extension, 10 nM ngRNA, and 100 ng mRNA). Guide RNAs and MS2-loop-linked 3′ extensions were heat shocked at 95 C for 3 minutes and immediately placed on ice for 5 minutes before dilution in Opti-MEM™ (Gibco, Cat. 31985-062) prior to transfection. Samples were run in duplicates. The cells were lysed 72 hours post-treatment for NGS analysis as described in Example 1.

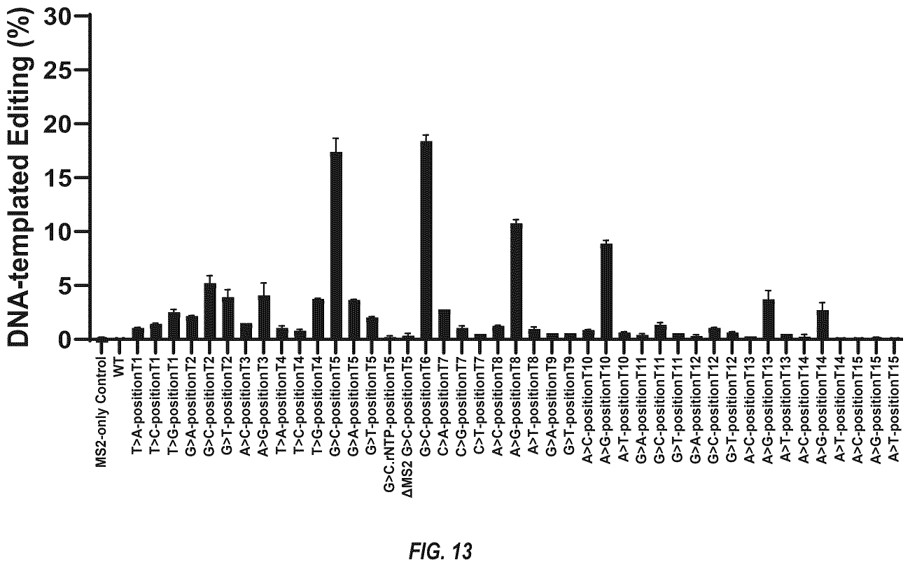

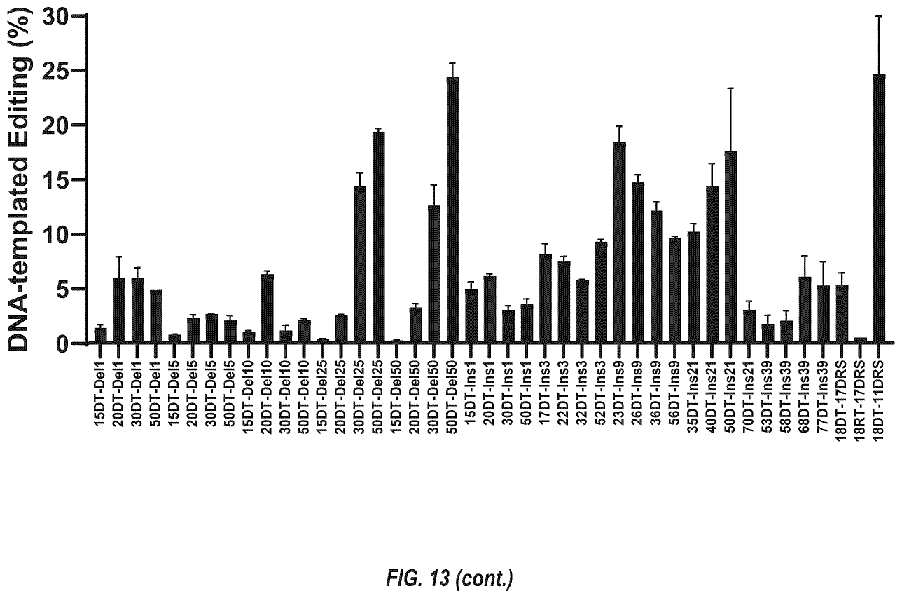

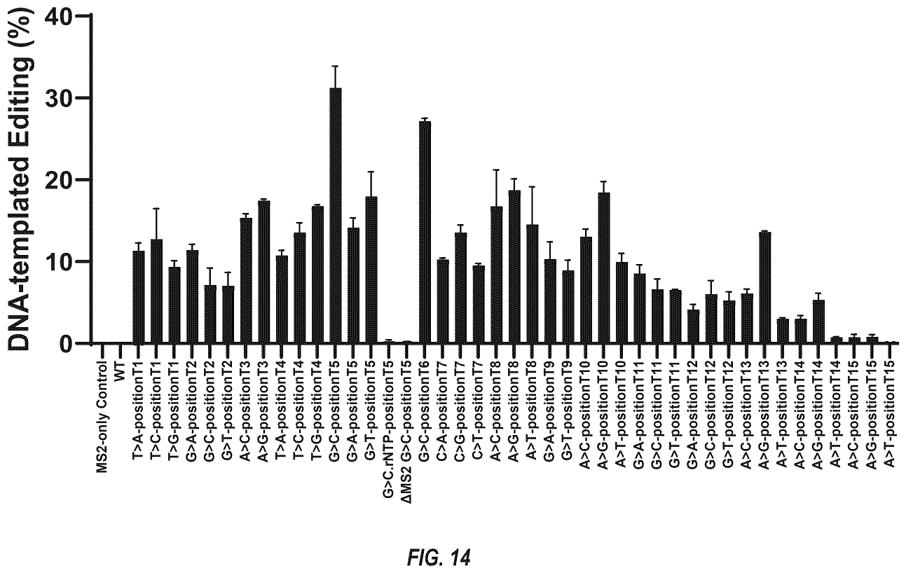

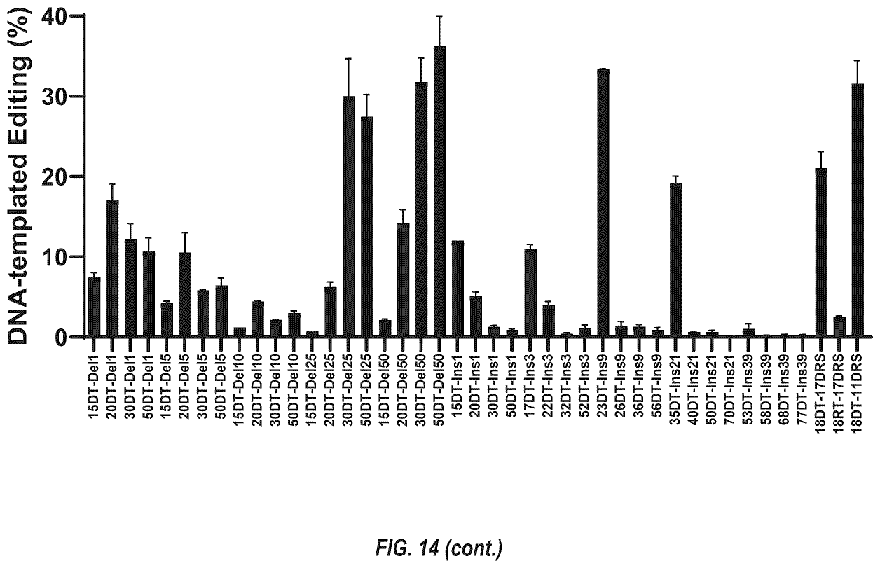

Mean templated editing results with standard deviation (SD) are shown in FIG. 13 and Table 15 for conditions treated without ngRNA. Mean templated editing results with standard deviation (SD) are shown in FIG. 14 and Table 16 for conditions treated with their corresponding ngRNA.

TABLE 15
Mean percent DNA-templated edits at the HEK3 or VEGFA locus
as encoded by MS2-loop-linked 3′ extensions in Huh7 cells
using MCP-PolK-nCas9 (SEQ ID NO: 1057) in the absence of ngRNA.

| MS2-loop- | MS2-loop- | Mean DNA- | |
|---|---|---|---|
| linked 3′ | linked 3′ | templated | |
| extensionID | extension alias | Editing (%) | SD |
| ST034737 | MS2-only Control | 0.00 | 0.00 |
| ST034738 | WT | 0.05 | 0.07 |
| ST034739 | T > A-positionT1 | 0.90 | 0.14 |
| ST034740 | T > C-positionT1 | 0.75 | 0.07 |
| ST034741 | T > G-positionT1 | 2.55 | 0.49 |
| ST034742 | G > A-positionT2 | 1.60 | 0.28 |
| ST034743 | G > C-positionT2 | 4.30 | 0.28 |
| ST034744 | G > T-positionT2 | 3.20 | 1.13 |
| ST034745 | A > C-positionT3 | 1.05 | 0.35 |
| ST034746 | A > G-positionT3 | 3.85 | 1.20 |
| ST034748 | T > A-positionT4 | 0.45 | 0.21 |
| ST034749 | T > C-positionT4 | 0.70 | 0.14 |
| ST034750 | T > G-positionT4 | 3.30 | 0.14 |
| ST034751 | G > C-positionT5 | 16.10 | 1.70 |
| ST034752 | G > A-positionT5 | 2.95 | 0.21 |
| ST034753 | G > T-positionT5 | 1.70 | 0.14 |
| ST034754 | G > C.rNTP-positionT5 | 0.00 | 0.00 |
| ST034755 | ΔMS2 G > C-positionT5 | 0.20 | 0.00 |
| ST034756 | G > C-positionT6 | 15.85 | 2.05 |
| ST034757 | C > A-positionT7 | 2.20 | 0.28 |
| ST034758 | C > G-positionT7 | 1.00 | 0.00 |
| ST034759 | C > T-positionT7 | 0.55 | 0.21 |
| ST034760 | A > C-positionT8 | 1.00 | 0.14 |
| ST034761 | A > G-positionT8 | 9.65 | 0.92 |
| ST034762 | A > T-positionT8 | 0.65 | 0.49 |
| ST034763 | G > A-positionT9 | 0.20 | 0.14 |
| ST034765 | G > T-positionT9 | 0.40 | 0.28 |
| ST034766 | A > C- | 1.05 | 0.21 |
| | positionT1positionT10 | | |
| ST034767 | A > G- | 8.55 | 0.07 |
| | positionT1positionT10 | | |
| ST034768 | A > T- | 0.40 | 0.28 |
| | positionT1positionT10 | | |
| ST034769 | G > A- | 0.35 | 0.21 |
| | positionT1positionT11 | | |
| ST034770 | G > C- | 1.05 | 0.21 |
| | positionT1positionT11 | | |
| ST034771 | G > T- | 0.45 | 0.07 |
| | positionT1positionT11 | | |
| ST034772 | G > A- | 0.30 | 0.14 |
| | positionT1positionT12 | | |
| ST034773 | G > C- | 0.55 | 0.07 |
| | positionT1positionT12 | | |
| ST034774 | G > T- | 0.25 | 0.07 |
| | positionT1positionT12 | | |
| ST034775 | A > C- | 0.20 | 0.14 |
| | positionT1positionT13 | | |
| ST034776 | A > G- | 3.55 | 0.35 |
| | positionT1positionT13 | | |
| ST034777 | A > T- | 0.10 | 0.14 |
| | positionT1positionT13 | | |
| ST034778 | A > C- | 0.00 | 0.00 |
| | positionT1positionT14 | | |
| ST034779 | A > G- | 2.60 | 0.85 |
| | positionT1positionT14 | | |
| ST034780 | A > T- | 0.00 | 0.00 |
| | positionT1positionT14 | | |
| ST034781 | A > C- | 0.00 | 0.00 |
| | positionT1positionT15 | | |
| ST034782 | A > G- | 0.05 | 0.07 |
| | positionT1positionT15 | | |
| ST034783 | A > T- | 0.00 | 0.00 |
| | positionT1positionT15 | | |
| ST034784 | 15DT-Del1 | 0.30 | 0.14 |
| ST034785 | 20DT-Del1 | 2.05 | 0.78 |
| ST034786 | 30DT-Del1 | 1.50 | 0.57 |
| ST034787 | 50DT-Del1 | 2.15 | 0.35 |
| ST034788 | 15DT-Del5 | 0.05 | 0.07 |
| ST034789 | 20DT-Del5 | 1.00 | 0.14 |
| ST034790 | 30DT-Del5 | 1.15 | 0.64 |
| ST034791 | 50DT-Del5 | 0.95 | 0.07 |
| ST034792 | 15DT-Del10 | 0.40 | 0.14 |
| ST034793 | 20DT-Del10 | 1.95 | 0.21 |
| ST034794 | 30DT-Del10 | 0.30 | 0.14 |
| ST034795 | 50DT-Del10 | 1.05 | 0.49 |
| ST034796 | 15DT-Del25 | 0.00 | 0.00 |
| ST034797 | 20DT-Del25 | 1.25 | 0.49 |
| ST034798 | 30DT-Del25 | 11.80 | 0.57 |
| ST034799 | 50DT-Del25 | 15.65 | 3.18 |
| ST034800 | 15DT-Del50 | 0.05 | 0.07 |
| ST034801 | 20DT-Del50 | 2.60 | 0.71 |
| ST034802 | 30DT-Del50 | 11.45 | 2.47 |
| ST034803 | 50DT-Del50 | 24.00 | 2.97 |
| ST034804 | 15DT-Ins1 | 2.75 | 0.21 |
| ST034805 | 20DT-Ins1 | 2.65 | 0.35 |
| ST034806 | 30DT-Ins1 | 0.50 | 0.14 |
| ST034807 | 50DT-Ins1 | 0.55 | 0.21 |
| ST034808 | 17DT-Ins3 | 3.95 | 0.78 |
| ST034809 | 22DT-Ins3 | 1.50 | 0.28 |
| ST034810 | 32DT-Ins3 | 0.30 | 0.00 |
| ST034811 | 52DT-Ins3 | 0.30 | 0.14 |
| ST034812 | 23DT-Ins9 | 22.50 | 2.55 |
| ST034813 | 26DT-Ins9 | 3.15 | 0.21 |
| ST034814 | 36DT-Ins9 | 0.90 | 0.57 |
| ST034815 | 56DT-Ins9 | 0.70 | 0.00 |
| ST034816 | 35DT-Ins21 | 16.00 | 0.99 |
| ST034817 | 40DT-Ins21 | 1.70 | 0.14 |
| ST034818 | 50DT-Ins21 | 0.70 | 0.14 |
| ST034819 | 70DT-Ins21 | 0.10 | 0.14 |
| ST034824 | 53DT-Ins39 | 1.90 | 1.27 |
| ST034825 | 58DT-Ins39 | 0.10 | 0.14 |
| ST034826 | 68DT-Ins39 | 0.15 | 0.21 |
| ST034827 | 77DT-Ins39 | 0.15 | 0.07 |
| ST034641 | 18DT-17DRS | 5.05 | 1.06 |
| ST034828 | 18RT-17DRS | 0.45 | 0.07 |
| ST034829 | 18DT-11DRS | 22.25 | 4.31 |

TABLE 16
Mean percent templated edits at the HEK3 or VEGFA locus
as encoded by template in Huh7 cells using MCP-PolK-
nCas9 (SEQ ID NO: 1057) in the presence of ngRNA.

| MS2-loop- | MS2-loop- | | Mean DNA- | |
|---|---|---|---|---|
| linked 3′- | linked 3′- | | templated | |
| extension ID | extension alias | ngRNA | Editing (%) | SD |
| ST034737 | MS2-only Control | G023020 | 0.00 | 0.00 |
| ST034738 | WT | G023020 | 0.00 | 0.00 |
| ST034739 | T > A-positionT1 | G023020 | 11.30 | 0.99 |
| ST034740 | T > C-positionT1 | G023020 | 12.75 | 3.75 |
| ST034741 | T > G-positionT1 | G023020 | 9.35 | 0.78 |
| ST034742 | G > A-positionT2 | G023020 | 11.40 | 0.71 |
| ST034743 | G > C-positionT2 | G023020 | 7.15 | 2.05 |
| ST034744 | G > T-positionT2 | G023020 | 7.05 | 1.63 |
| ST034745 | A > C-positionT3 | G023020 | 15.35 | 0.49 |
| ST034746 | A > G-positionT3 | G023020 | 17.45 | 0.21 |
| ST034748 | T > A-positionT4 | G023020 | 10.75 | 0.64 |
| ST034749 | T > C-positionT4 | G023020 | 13.55 | 1.20 |
| ST034750 | T > G-positionT4 | G023020 | 16.80 | 0.14 |
| ST034751 | G > C-positionT5 | G023020 | 31.20 | 2.69 |
| ST034752 | G > A-positionT5 | G023020 | 14.15 | 1.20 |
| ST034753 | G > T-positionT5 | G023020 | 17.95 | 3.04 |
| ST034754 | G > C.rNTP- | G023020 | 0.25 | 0.21 |
| | positionT5 | | | |
| ST034755 | ΔMS2 G > C- | G023020 | 0.10 | 0.14 |
| | positionT5 | | | |
| ST034756 | G > C-positionT6 | G023020 | 27.15 | 0.35 |
| ST034757 | C > A-positionT7 | G023020 | 10.25 | 0.21 |
| ST034758 | C > G-positionT7 | G023020 | 13.55 | 0.92 |
| ST034759 | C > T-positionT7 | G023020 | 9.50 | 0.28 |
| ST034760 | A > C-positionT8 | G023020 | 16.75 | 4.45 |
| ST034761 | A > G-positionT8 | G023020 | 18.70 | 1.41 |
| ST034762 | A > T-positionT8 | G023020 | 14.55 | 4.60 |
| ST034763 | G > A-positionT9 | G023020 | 10.30 | 2.12 |
| ST034765 | G > T-positionT9 | G023020 | 8.90 | 1.27 |
| ST034766 | A > C- | G023020 | 13.05 | 0.92 |
| | positionT1positionT10 | | | |
| ST034767 | A > G- | G023020 | 18.45 | 1.34 |
| | positionT1positionT10 | | | |
| ST034768 | A > T- | G023020 | 9.95 | 1.06 |
| | positionT1positionT10 | | | |
| ST034769 | G > A- | G023020 | 8.55 | 1.06 |
| | positionT1positionT11 | | | |
| ST034770 | G > C- | G023020 | 6.60 | 1.27 |
| | positionT1positionT11 | | | |
| ST034771 | G > T- | G023020 | 6.55 | 0.07 |
| | positionT1positionT11 | | | |
| ST034772 | G > A- | G023020 | 4.15 | 0.64 |
| | positionT1positionT12 | | | |
| ST034773 | G > C- | G023020 | 6.00 | 1.70 |
| | positionT1positionT12 | | | |
| ST034774 | G > T- | G023020 | 5.25 | 1.06 |
| | positionT1positionT12 | | | |
| ST034775 | A > C- | G023020 | 6.10 | 0.57 |
| | positionT1positionT13 | | | |
| ST034776 | A > G- | G023020 | 13.60 | 0.14 |
| | positionT1positionT13 | | | |
| ST034777 | A > T- | G023020 | 3.00 | 0.14 |
| | positionT1positionT13 | | | |
| ST034778 | A > C- | G023020 | 3.00 | 0.42 |
| | positionT1positionT14 | | | |
| ST034779 | A > G- | G023020 | 5.30 | 0.85 |
| | positionT1positionT14 | | | |
| ST034780 | A > T- | G023020 | 0.75 | 0.07 |
| | positionT1positionT14 | | | |
| ST034781 | A > C- | G023020 | 0.75 | 0.35 |
| | positionT1positionT15 | | | |
| ST034782 | A > G- | G023020 | 0.80 | 0.28 |
| | positionT1positionT15 | | | |
| ST034783 | A > T- | G023020 | 0.10 | 0.00 |
| | positionT1positionT15 | | | |
| ST034784 | 15DT-Del1 | G023020 | 7.55 | 0.49 |
| ST034785 | 20DT-Del1 | G023020 | 17.10 | 1.98 |
| ST034786 | 30DT-Del1 | G023020 | 12.25 | 1.91 |
| ST034787 | 50DT-Del1 | G023020 | 10.75 | 1.63 |
| ST034788 | 15DT-Del5 | G023020 | 4.20 | 0.28 |
| ST034789 | 20DT-Del5 | G023020 | 10.55 | 2.47 |
| ST034790 | 30DT-Del5 | G023020 | 5.85 | 0.07 |
| ST034791 | 50DT-Del5 | G023020 | 6.45 | 0.92 |
| ST034792 | 15DT-Del10 | G023020 | 1.20 | 0.00 |
| ST034793 | 20DT-Del10 | G023020 | 4.45 | 0.07 |
| ST034794 | 30DT-Del10 | G023020 | 2.15 | 0.07 |
| ST034795 | 50DT-Del10 | G023020 | 3.00 | 0.28 |
| ST034796 | 15DT-Del25 | G023020 | 0.70* | N/A |
| ST034797 | 20DT-Del25 | G023020 | 6.25 | 0.64 |
| ST034798 | 30DT-Del25 | G023020 | 30.00 | 4.67 |
| ST034799 | 50DT-Del25 | G023020 | 27.45 | 2.76 |
| ST034800 | 15DT-Del50 | G023020 | 2.10 | 0.14 |
| ST034801 | 20DT-Del50 | G023020 | 14.20 | 1.70 |
| ST034802 | 30DT-Del50 | G023020 | 31.80 | 2.97 |
| ST034803 | 50DT-Del50 | G023020 | 36.25 | 3.75 |
| ST034804 | 15DT-Ins1 | G023020 | 12.00 | 0.00 |
| ST034805 | 20DT-Ins1 | G023020 | 5.15 | 0.49 |
| ST034806 | 30DT-Ins1 | G023020 | 1.30 | 0.14 |
| ST034807 | 50DT-Ins1 | G023020 | 0.90 | 0.14 |
| ST034808 | 17DT-Ins3 | G023020 | 11.00 | 0.57 |
| ST034809 | 22DT-Ins3 | G023020 | 3.95 | 0.49 |
| ST034810 | 32DT-Ins3 | G023020 | 0.40 | 0.14 |
| ST034811 | 52DT-Ins3 | G023020 | 1.10 | 0.42 |
| ST034812 | 23DT-Ins9 | G023020 | 33.35 | 0.07 |
| ST034813 | 26DT-Ins9 | G023020 | 1.45 | 0.49 |
| ST034814 | 36DT-Ins9 | G023020 | 1.30 | 0.28 |
| ST034815 | 56DT-Ins9 | G023020 | 0.90 | 0.28 |
| ST034816 | 35DT-Ins21 | G023020 | 19.20 | 0.85 |
| ST034817 | 40DT-Ins21 | G023020 | 0.65 | 0.07 |
| ST034818 | 50DT-Ins21 | G023020 | 0.65 | 0.21 |
| ST034819 | 70DT-Ins21 | G023020 | 0.10 | 0.00 |
| ST034824 | 53DT-Ins39 | G023020 | 1.05 | 0.64 |
| ST034825 | 58DT-Ins39 | G023020 | 0.10 | 0.14 |
| ST034826 | 68DT-Ins39 | G023020 | 0.20 | 0.14 |
| ST034827 | 77DT-Ins39 | G023020 | 0.25 | 0.07 |
| ST034641 | 18DT-17DRS | G034622 | 21.05 | 2.05 |
| ST034828 | 18RT-17DRS | G034622 | 2.50 | 0.14 |
| ST034829 | 18DT-11DRS | G034622 | 31.55 | 2.90 |

*Indicates experimental group for which N = 1

### Example 5—Indel Analysis Using Chemically Modified Guide RNAs with Various DRS and Template Modifications

This Example evaluated genome editing efficiency with tgRNAs having various chemical modifications. In particular, the impact of varying numbers of 2′-O-methyl modified nucleotides at the 3′ end of the tgRNA (e.g., in the DNA polymerase recruiting sequence; DRS) was assessed. This Example further evaluated whether guides comprising a 3′ extension, with or without modifications, maintain the capacity to generate indels when used with a SpyCas9 cleavase. See Table 17 for a description of the guide RNAs evaluated in this Example.

Guide RNAs targeting the same target sequence in the HEK3 genomic locus with 3′ extensions encoding a G-to-C transversion mutation were designed with varying numbers of 2′-O-methyl modified nucleotides at the 3′ end of the nucleic acid. Table 17 shows the 3′ extension sequences of these guide nucleic acids where lowercase ‘m’ preceding a nucleotide base indicates a 2′ O-methyl modification to that base. The guides were lipofected into human hepatoma (Huh7) cells alongside a SpyCas9 cleavase mRNA (SEQ ID NO: 1001). Editing efficiency of the guides was determined as follows.

TABLE 17
Chemical modification of 3′ guide RNA extension

| Guide ID | Extension Sequence (DRS in bold; template region in italics) |
|---|---|
| G029546 | UCUGCGAUCACGUGCUCAGUCUG |
| (SEQ ID NO: 509) | (SEQ ID NO: 924) |
| G029547 | UCUGCGAUCACGUGCUCAGUCU*G |
| (SEQ ID NO: 510) | (SEQ ID NO: 925) |
| G029548 | UCUGCGAUCACGUGCUCAGUCUmG |
| (SEQ ID NO: 511) | (SEQ ID NO: 926) |
| G029549 | UCUGCGAUCACGUGCUCAGUCU*mG |
| (SEQ ID NO: 512) | (SEQ ID NO: 927) |
| G029550 | UCUGCGAUCACGUGCUCAGUCmU*mG |
| (SEQ ID NO: 513) | (SEQ ID NO: 928) |
| G029551 | UCUGCGAUCACGUGCUCAGUmCmU*mG |
| (SEQ ID NO: 514) | (SEQ ID NO: 929) |
| G029552 | UCUGCGAUCACGUGCUCAGmUmCmU*mG |
| (SEQ ID NO: 515) | (SEQ ID NO: 930) |
| G029553 | UCUGCGAUCACGUGCUCAmGmUmCmU*mG |
| (SEQ ID NO: 516) | (SEQ ID NO: 931) |
| G029554 | UCUGCGAUCACGUGCUCmAmGmUmCmU*mG |
| (SEQ ID NO: 517) | (SEQ ID NO: 932) |
| G029555 | UCUGCGAUCACGUGCUmCmAmGmUmCmU*mG |
| (SEQ ID NO: 518) | (SEQ ID NO: 933) |
| G029556 | UCUGCGAUCACGUGCmUmCmAmGmUmCmU*mG |
| (SEQ ID NO: 415) | (SEQ ID NO: 934) |
| G029557 | UCUGCGAUCACGUGmCmUmCmAmGmUmCmU*mG |
| (SEQ ID NO: 519) | (SEQ ID NO: 935) |
| G029558 | UCUGCGAUCACGUmGmCmUmCmAmGmUmCmU*mG |
| (SEQ ID NO: 520 | (SEQ ID NO: 936) |
| G029559 | UCUGCGAUCACGmUmGmCmUmCmAmGmUmCmU*mG |
| (SEQ ID NO: 521) | (SEQ ID NO: 937) |
| G029560 | UCUGCGAUCACmGmUmGmCmUmCmAmGmUmCmU*mG |
| (SEQ ID NO: 522) | (SEQ ID NO: 938) |
| G029561 | UCUGCGAUCAmCmGmUmGmCmUmCmAmGmUmCmU*mG |
| (SEQ ID NO: 523) | (SEQ ID NO: 939) |
| G029562 | UCUGCGAUCmAmCmGmUmGmCmUmCmAmGmUmCmU*mG |
| (SEQ ID NO: 524) | (SEQ ID NO: 940) |
| G029563 | UCUGCGAUmCmAmCmGmUmGmCmUmCmAmGmUmCmU*mG |
| (SEQ ID NO: 525) | (SEQ ID NO: 941) |

Note:
*denotes that the nucleotide is linked to the next nucleotide with a phosphorothioate (PS bond), a lower case “m” denotes that the nucleotide is modified with 2′-O-Me, and a lowercase “d” denotes that the nucleotide is a DNA nucleotide.

Cells were prepared as described in Example 1, plated at a density of 15,000 cells/well, and incubated for 24 hours at 37° C. and 5% CO₂ atmosphere.

Lipofectamine™ MessengerMAX™ Reagent (Thermo Fisher, Cat. LMRNA001) was used and samples were prepared according to the manufacturer's protocol with 100 ng of SpyCas9 mRNA and 50 nM guide per transfection well. Guide RNAs were prepared either as single-molecule tgRNAs or as mixtures of a tgRNA with a nicking guide RNA (G023020) in a 1:1 molar ratio. Samples were run in duplicate. The cells were lysed 72 hours post-treatment for NGS analysis as described in Example 1.

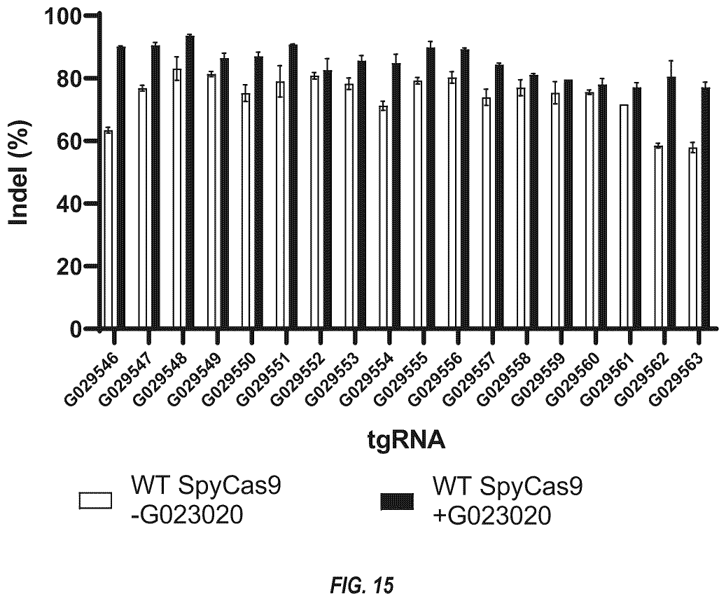

Mean percent indel results with standard deviation (SD) are shown in Table 18 and FIG. 15.

TABLE 18
Percent indels at the HEK3 locus in Huh7 cells using
SpyCas9 mRNA and a tgRNA with or without a ngRNA.

| | gRNA | Nicking | ngRNA | Mean Indel | |
|---|---|---|---|---|---|
| Guide ID | (nM) | Guide | (nM) | (%) | SD |
| G029546 | 50 | — | 0 | 63.5 | 0.9 |
| G029547 | 50 | — | 0 | 76.9 | 0.9 |
| G029548 | 50 | — | 0 | 83.2 | 3.7 |
| G029549 | 50 | — | 0 | 81.4 | 0.8 |
| G029550 | 50 | — | 0 | 75.3 | 2.7 |
| G029551 | 50 | — | 0 | 79.1 | 5.0 |
| G029552 | 50 | — | 0 | 80.9 | 1.1 |
| G029553 | 50 | — | 0 | 78.3 | 1.8 |
| G029554 | 50 | — | 0 | 71.3 | 1.5 |
| G029555 | 50 | — | 0 | 79.3 | 1.1 |
| G029556 | 50 | — | 0 | 80.3 | 1.8 |
| G029557 | 50 | — | 0 | 74.0 | 2.6 |
| G029558 | 50 | — | 0 | 77.0 | 2.5 |
| G029559 | 50 | — | 0 | 75.4 | 3.5 |
| G029560 | 50 | — | 0 | 75.6 | 0.7 |
| G029561 | 50 | — | 0 | 71.7 | 0.0 |
| G029562 | 50 | — | 0 | 58.6 | 0.8 |
| G029563 | 50 | — | 0 | 58.0 | 1.6 |
| G029546 | 25 | G023020 | 25 | 90.2 | 0.2 |
| G029547 | 25 | G023020 | 25 | 90.6 | 0.9 |
| G029548 | 25 | G023020 | 25 | 93.7 | 0.4 |
| G029549 | 25 | G023020 | 25 | 86.5 | 1.6 |
| G029550 | 25 | G023020 | 25 | 87.0 | 1.4 |
| G029551 | 25 | G023020 | 25 | 90.8 | 0.2 |
| G029552 | 25 | G023020 | 25 | 82.6 | 3.7 |
| G029553 | 25 | G023020 | 25 | 85.6 | 1.7 |
| G029554 | 25 | G023020 | 25 | 84.9 | 2.9 |
| G029555 | 25 | G023020 | 25 | 89.9 | 1.9 |
| G029556 | 25 | G023020 | 25 | 89.3 | 0.4 |
| G029557 | 25 | G023020 | 25 | 84.4 | 0.5 |
| G029558 | 25 | G023020 | 25 | 81.1 | 0.4 |
| G029559 | 25 | G023020 | 25 | 79.6 | 0.0 |
| G029560 | 25 | G023020 | 25 | 78.0 | 2.0 |
| G029561 | 25 | G023020 | 25 | 77.2 | 1.5 |
| G029562 | 25 | G023020 | 25 | 80.5 | 5.1 |
| G029563 | 25 | G023020 | 25 | 77.2 | 1.6 |

### Example 6—Template Based Editing in Huh7 Cells with Various DNA-Dependent DNA Polymerases (Klenow, DNA Polymerase θ, and T5 DNA Polymerase; Including Variants) with Various DNA/RNA Composition in the 3′ Extension

This Example evaluated templated editing efficiency with tgRNAs having varied DNA/RNA compositions in the 3′ extension region of the tgRNA. Further, single polypeptide fusions were evaluated that included a SpyCas9 nickase (nCas9) operably linked to different DNA polymerases (i.e., Klenow(exo-), PolQ, or T5). In particular, the following single polypeptide fusions were assessed: nCas9-Klenow(exo-) (SEQ ID NO: 1110); nCas9-polQ(1819-2590) (SEQ ID NO: 1082); and nCas9-T5 Pol(D164A,E166A (SEQ ID NO: 1130). Each Cas9/DNA polymerase fusion was evaluated in complex with a tgRNA having a chimeric 3′ extension region (e.g., with varying DNA/RNA compositions) and assessed for in vitro templated editing. See Table 19A for a description of the guide RNAs evaluated in this Example.

Template guide RNAs (tgRNAs) comprising the same 20-nucleotide spacer sequence targeting the HEK3 genomic locus were designed with 3′ extensions containing different DNA/RNA compositions. The tgRNAs were used for editing with or without an additional nicking guide RNA (G025992; SEQ ID NO: 481), which targets the opposite strand of the DNA duplex target nucleic acid from the tgRNA, 90 bp away from the cut site.

Cells were prepared as described in Example 1, plated at a density of 15,000 cells/well and incubated at 37° C. and 5% CO₂ atmosphere for 24 hours.

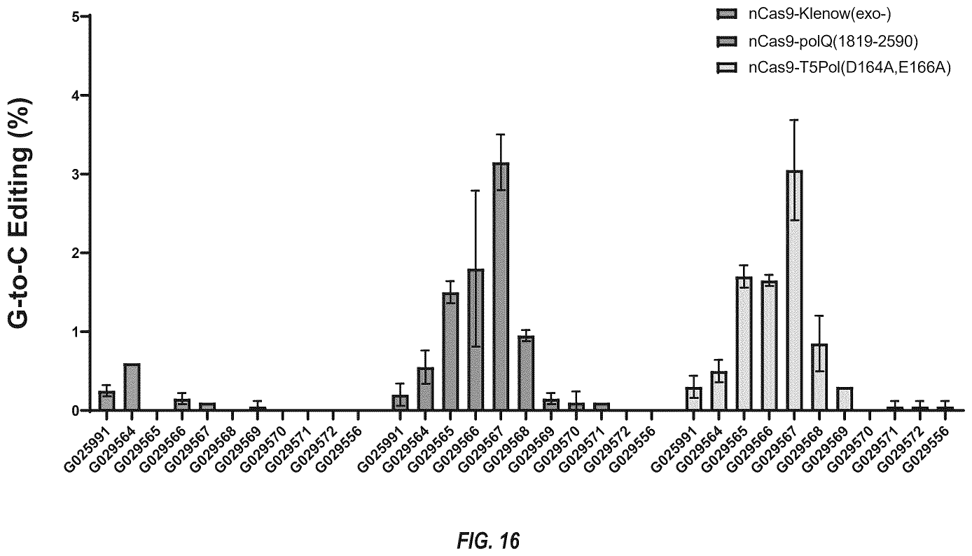

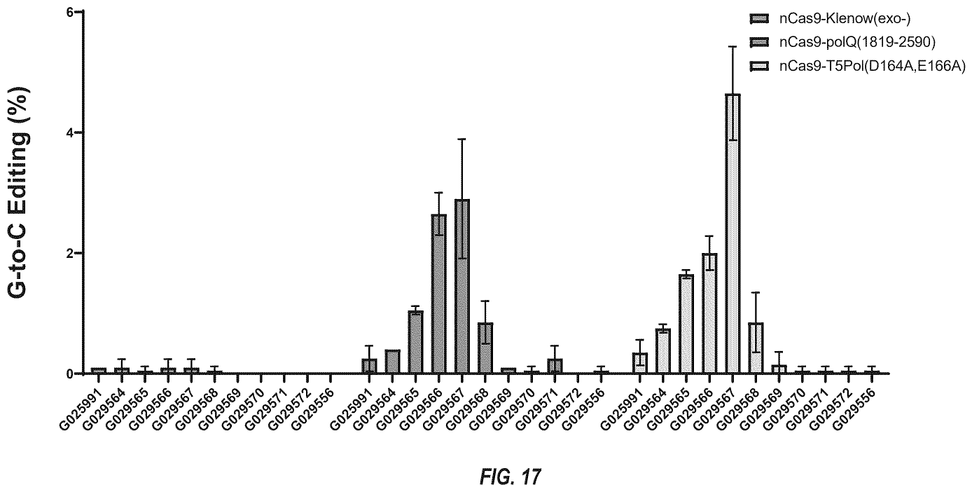

mRNAs encoding a H840A SpyCas9 nickase fused to a DNA-dependent DNA polymerase and guide RNAs were delivered using Lipofectamine™ MessengerMAX™ Reagent (Thermo Fisher, Cat. LMRNA001) according to the manufacturer's protocol. Lipoplexes containing 100 ng of mRNA or 25 nM guide RNA were formed then delivered simultaneously. Guide lipoplexes contained either 25 nM of tgRNA, or 25 nM of tgRNA plus ngRNA in a molar ratio of 3:2 tgRNA:ngRNA. Samples were run in duplicates. Cells were lysed 72 hours post-treatment for NGS analysis as described in Example 1. Mean percent templated G-to-C transversions, with standard deviation (SD) are shown in Tables 19B and 20, and FIGS. 16 and 17 below.

TABLE 19A
Description of guide RNA Constructs

| Guide ID | Description of Guide RNA-3′ Extension Sequence (DRS in bold) |
|---|---|
| G025991 (SEQ ID | dTdCdTdGdCdGdAdTdCdAdCdGdTdGdCdTdCdAdGdTdT*dT*dG |
| NO: 493) | (C7NH2) (SEQ ID NO: 912) |
| G029564 (SEQ ID | dTdCdTdGdCdGdAdTdCdAdCdGdTdGdCdTdCmAmGmUmCmU*mG |
| NO: 494) | (SEQ ID NO: 913) |
| G029565 (SEQ ID | dTdCdTdGdCdGdAdTdCdAdCdGdTGCUCmAmGmUmCmU*mG (SEQ |
| NO: 495) | ID NO: 914) |
| G029566 (SEQ ID | dTdCdTdGdCdGdAdTdCdAdCdGUGCUCmAmGmUmCmU*mG (SEQ ID |
| NO: 496) | NO: 915) |
| G029567 (SEQ ID | dTdCdTdGdCdGdAdTdCdAdCGUGCUCmAmGmUmCmU*mG (SEQ ID |
| NO: 497) | NO: 916) |
| G029568 (SEQ ID | dTdCdTdGdCdGdAdTdCdACGUGCUCmAmGmUmCmU*mG (SEQ ID |
| NO: 498) | NO: 917) |
| G029569 (SEQ ID | dTdCdTdGdCdGdAdTdCACGUGCUCmAmGmUmCmU*mG (SEQ ID NO: |
| NO: 499) | 918) |
| G029570 (SEQ ID | dTdCdTdGdCdGdAdTCACGUGCUCmAmGmUmCmU*mG (SEQ ID NO: |
| NO: 500) | 919) |
| G029571 (SEQ ID | dTdCdTGdCdGdAUCACGUGCUCmAmGmUmCmU*mG (SEQ ID NO: |
| NO: 501) | 920) |
| G029572 (SEQ ID | dTdCdTdGCGAUCACGUGCUCmAmGmUmCmU*mG (SEQ ID NO: 921) |
| NO: 502) | |
| G029556 (SEQ ID | UCUGCGAUCACGUGCmUmCmAmGmUmCmU*mG (SEQ ID NO: 922) |
| NO: 415) | |

Note:
*denotes that the nucleotide is linked to the next nucleotide with a phosphorothioate (PS bond), a lower case “m” denotes that the nucleotide is modified with 2′-O-Me, and a lowercase “d” denotes that the nucleotide is a DNA nucleotide.

TABLE 19B
Percent templated G-to-C editing at the HEK3 locus in the absence of ngRNA

| | mRNA ID | | | | | |
|---|---|---|---|---|---|---|
| | nCas9-Klenow(exo-) | | nCas9-polQ(1819-2590) | | nCas9-T5Pol(D164A, E166A) | |
| | (SEQ ID NO: 1110) | | (SEQ ID NO: 1082) | | (SEQ ID NO: 1130) | |
| | Mean G-to-C | | Mean G-to-C | | Mean G-to-C | |
| Guide ID | Editing (%) | SD | Editing (%) | SD | Editing (%) | SD |
| G025991 | 0.25 | 0.07 | 0.20 | 0.14 | 0.30 | 0.14 |
| G029564 | 0.60 | 0.00 | 0.55 | 0.21 | 0.50 | 0.14 |
| G029565 | 0.00 | 0.00 | 1.50 | 0.14 | 1.70 | 0.14 |
| G029566 | 0.15 | 0.07 | 1.80 | 0.99 | 1.65 | 0.07 |
| G029567 | 0.10 | 0.00 | 3.15 | 0.35 | 3.05 | 0.64 |
| G029568 | 0.00 | 0.00 | 0.95 | 0.07 | 0.85 | 0.35 |
| G029569 | 0.05 | 0.07 | 0.15 | 0.07 | 0.30 | 0.00 |
| G029570 | 0.00 | 0.00 | 0.10 | 0.14 | 0.00 | 0.00 |
| G029571 | 0.00 | 0.00 | 0.10 | 0.00 | 0.05 | 0.07 |
| G029572 | 0.00 | 0.00 | 0.00 | 0.00 | 0.05 | 0.07 |
| G029556 | 0.00 | 0.00 | 0.00 | 0.00 | 0.05 | 0.07 |

TABLE 20
Templated G-to-C editing at the HEK3 locus in the presence of ngRNA (G025992)

| | mRNA ID | | | | | |
|---|---|---|---|---|---|---|
| | nCas9-Klenow(exo-) | | nCas9-polQ(1819-2590) | | nCas9-T5Pol(D164A, E166A) | |
| | (SEQ ID NO: 1110) | | (SEQ ID NO: 1082) | | (SEQ ID NO: 1130) | |
| | Mean G-to-C | | Mean G-to-C | | Mean G-to-C | |
| | Editing (%) | SD | Editing (%) | SD | Editing (%) | SD |
| G025991 | 0.10 | 0.00 | 0.25 | 0.21 | 0.35 | 0.21 |
| G029564 | 0.10 | 0.14 | 0.40 | 0.00 | 0.75 | 0.07 |
| G029565 | 0.05 | 0.07 | 1.05 | 0.07 | 1.65 | 0.07 |
| G029566 | 0.10 | 0.14 | 2.65 | 0.35 | 2.00 | 0.28 |
| G029567 | 0.10 | 0.14 | 2.90 | 0.99 | 4.65 | 0.78 |
| G029568 | 0.05 | 0.07 | 0.85 | 0.35 | 0.85 | 0.49 |
| G029569 | 0.00 | 0.00 | 0.10 | 0.00 | 0.15 | 0.21 |
| G029570 | 0.00 | 0.00 | 0.05 | 0.07 | 0.05 | 0.07 |
| G029571 | 0.00 | 0.00 | 0.25 | 0.21 | 0.05 | 0.07 |
| G029572 | 0.00 | 0.00 | 0.00 | 0.00 | 0.05 | 0.07 |
| G029556 | 0.00 | 0.00 | 0.05 | 0.07 | 0.05 | 0.07 |

### Example 7—Template Based Editing in Huh7 Cells with Various DNA-Dependent DNA Polymerases (DNA Polymerase K, DNA Polymerase θ, and T5 DNA Polymerase; Including Variants)

This Example evaluated templated editing efficiency with single polypeptide fusions including a SpyCas9 nickase (nCas9) operably linked to different DNA polymerases (PolK, T5, and PolQ). In particular, the following single polypeptide fusions were assessed: polK-nCas9 (SEQ ID NO: 1025); nCas9-T5Pol(D164A,E166A) (SEQ ID NO: 1130) and nCas9-polQ(1819-2590) (SEQ ID NO: 1082). Each Cas9/DNA polymerase fusion was complexed with a tgRNA having a chimeric RNA/DNA 3′ extension and assessed for in vitro template-based editing.

A template guide RNA (tgRNA) comprising a 20-nucleotide spacer sequence targeting the HEK3 genomic locus was designed with a chimeric RNA/DNA 3′ extension (G031155; SEQ ID NO: 503) encoding a G-to-C transversion. An additional ngRNA (G025992), which targets the opposite strand of the DNA duplex target nucleic acid from the tgRNA, 90 bp away from the cut site, was used. Guides were delivered into human hepatoma (Huh7) cells along with mRNAs encoding a SpyCas9 H840A nickase fused to a polymerase, and editing efficiency was evaluated by NGS as described in Example 1.

Cells were prepared as described in Example 1, plated at a density of 15,000 cells/well and incubated at 37° C. and 5% CO₂ atmosphere for 24 hours.

LNPs were prepared as described in Example 1, with a molar ratio of 50% Lipid A, 38% cholesterol, 9% DSPC, and 3% PEG2k-DMG, and a lipid amine to RNA phosphate (N: P) molar ratio of about 6. Each LNP comprised a single RNA cargo. LNP containing mRNA and LNP containing guide RNA were delivered simultaneously at a ratio of 2:1 by weight, respectively. When ngRNA was added, the LNP containing ngRNA was delivered simultaneously along with the LNP containing mRNA and the LNP containing tgRNA, wherein the total guide RNA (tgRNA+ngRNA) to mRNA weight ratio remained 2:1, and the tgRNA:ngRNA G031155 ratio was 3:2 by weight. Total RNA cargo dose corresponded to the combined weight of guide RNA and mRNA species. Total RNA cargo was diluted in a 2-fold 6-point dilution series. LNPs were diluted in cell culture media with additional 10% FBS, incubated at 37° C. for 10 minutes and then added to the cells. All conditions were carried out in duplicates.

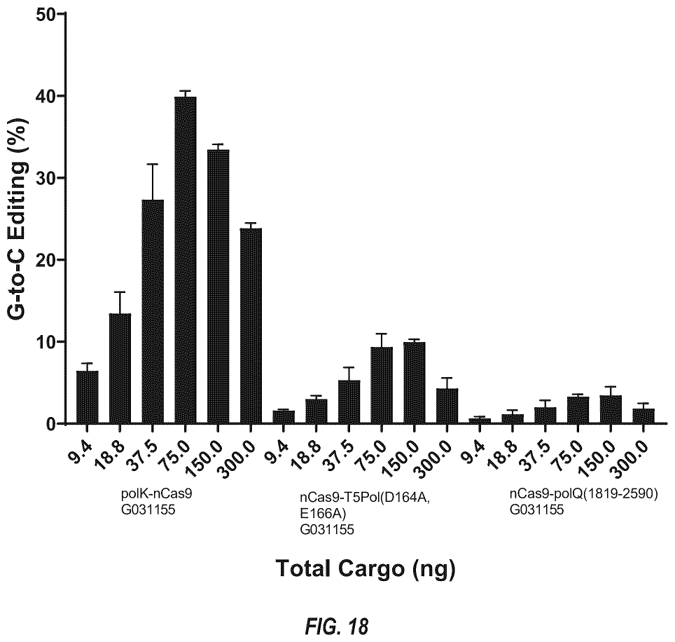

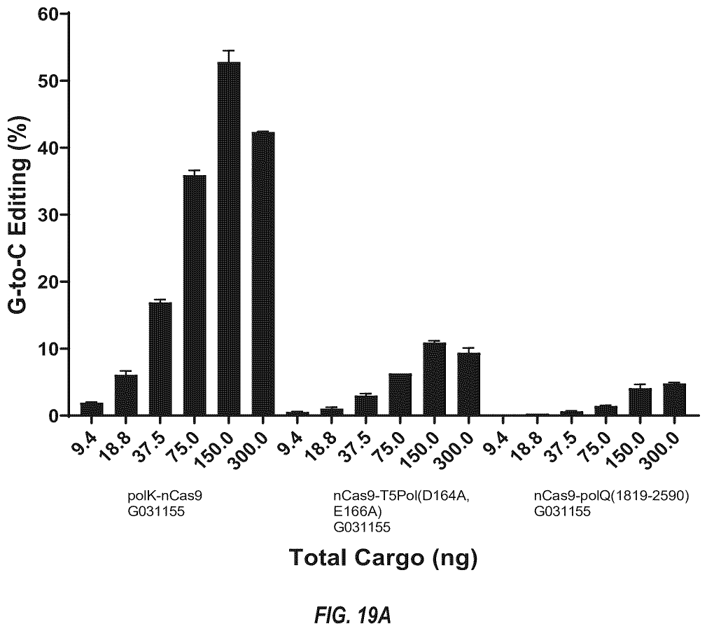

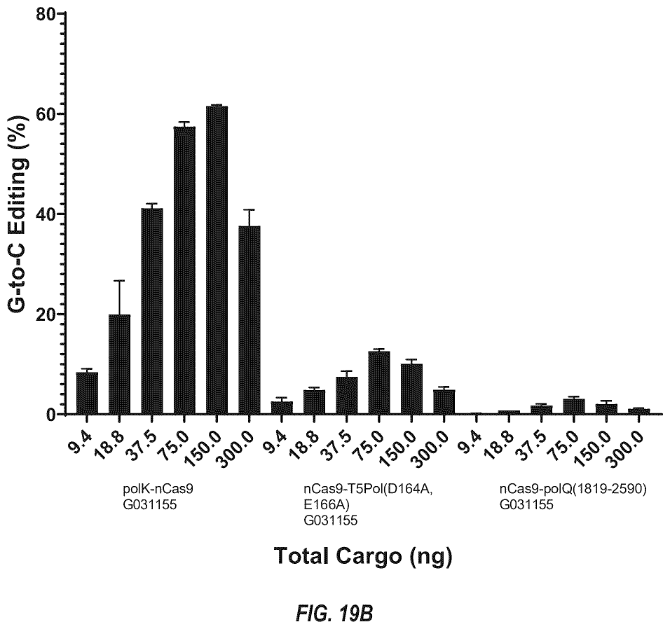

Cells were lysed 3 or 6 days post-treatment for NGS analysis as described in Example 1. Mean percent templated G-to-C transversions, with standard deviation (SD) are shown in Tables 21 and 22, and FIGS. 18, 19A, 19B, and 20A.

TABLE 21
Templated editing at the HEK3 locus in Huh7 cells with
LNP delivery of RNA species 3 days post-treatment

| LNP Delivery | | | | |
|---|---|---|---|---|
| | | Cargo | | |
| | | Dose | G-to-C Editing (%) | |
| mRNA ID | gRNA | (ng RNA) | Mean | SD |
| polK-nCas9 (SEQ ID | G031155 | 300 | 23.85 | 0.64 |
| NO: 1025) | | 150 | 33.45 | 0.64 |
| | | 75 | 39.90 | 0.71 |
| | | 37.5 | 27.35 | 4.31 |
| | | 18.75 | 13.45 | 2.62 |
| | | 9.38 | 6.45 | 0.92 |
| nCas9-T5Pol(D164A, | G031155 | 300 | 4.30 | 1.27 |
| E166A) (SEQ ID NO: | | 150 | 9.95 | 0.35 |
| 1130) | | 75 | 9.35 | 1.63 |
| | | 37.5 | 5.30 | 1.56 |
| | | 18.75 | 3.00 | 0.42 |
| | | 9.38 | 1.60 | 0.14 |
| nCas9-polQ(1819-2590) | G031155 | 300 | 1.85 | 0.64 |
| (SEQ ID NO: 1082) | | 150 | 3.45 | 1.06 |
| | | 75 | 3.30 | 0.28 |
| | | 37.5 | 2.00 | 0.85 |
| | | 18.75 | 1.15 | 0.49 |
| | | 9.38 | 0.65 | 0.21 |
| polK-nCas9 (SEQ ID | G031155 + | 300 | 37.60 | 3.25 |
| NO: 1025) | G025992 | 150 | 61.55 | 0.21 |
| | | 75 | 57.45 | 0.92 |
| | | 37.5 | 41.15 | 0.92 |
| | | 18.75 | 19.95 | 6.72 |
| | | 9.38 | 8.40 | 0.71 |
| nCas9-T5Pol(D164A, | G031155 + | 300 | 4.90 | 0.57 |
| E166A) (SEQ ID NO: | G025992 | 150 | 10.10 | 0.85 |
| 1130) | | 75 | 12.60 | 0.42 |
| | | 37.5 | 7.50 | 1.13 |
| | | 18.75 | 4.85 | 0.49 |
| | | 9.38 | 2.55 | 0.78 |
| nCas9-polQ(1819-2590) | G031155 + | 300 | 1.10 | 0.14 |
| (SEQ ID NO: 1082) | G025992 | 150 | 2.05 | 0.64 |
| | | 75 | 3.10 | 0.42 |
| | | 37.5 | 1.75 | 0.35 |
| | | 18.75 | 0.80 | 0.00 |
| | | 9.38 | 0.15 | 0.07 |

TABLE 22
Templated editing at the HEK3 locus in Huh7 cells with
LNP delivery of RNA species 6 days post-treatment

| LNP Delivery | | | G-to-C | |
|---|---|---|---|---|
| | | Cargo Dose | Editing (%) | |
| mRNA ID | gRNA | (ng RNA) | Mean | SD |
| polK-nCas9 (SEQ ID NO: | G031155 | 300 | 42.35 | 0.07 |
| 1025) | | 150 | 52.80 | 1.70 |
| | | 75 | 35.90 | 0.71 |
| | | 37.5 | 16.90 | 0.42 |
| | | 18.75 | 6.10 | 0.57 |
| | | 9.38 | 1.95 | 0.07 |
| nCas9-T5Pol(D164A, | G031155 | 300 | 9.40 | 0.71 |
| E166A) (SEQ ID NO: | | 150 | 10.90 | 0.28 |
| 1130) | | 75 | 6.30 | 0.00 |
| | | 37.5 | 3.00 | 0.28 |
| | | 18.75 | 1.05 | 0.21 |
| | | 9.38 | 0.55 | 0.07 |
| nCas9-polQ(1819-2590) | G031155 | 300 | 4.80 | 0.14 |
| (SEQ ID NO: 1082) | | 150 | 4.10 | 0.57 |
| | | 75 | 1.45 | 0.07 |
| | | 37.5 | 0.65 | 0.07 |
| | | 18.75 | 0.30 | 0.00 |
| | | 9.38 | 0.10 | 0.00 |
| polK-nCas9 (SEQ ID NO: | G031155 + | 300 | 68.60 | 2.97 |
| 1025) | G025992 | 150 | 76.70 | 0.14 |
| | | 75 | 53.45 | 4.17 |
| | | 37.5 | 23.20 | 0.42 |
| | | 18.75 | 6.25 | 0.21 |
| | | 9.38 | 1.85 | 0.07 |
| nCas9-T5Pol(D164A, | G031155 + | 300 | 11.00 | 1.56 |
| E166A) (SEQ ID NO: | G025992 | 150 | 12.25 | 0.35 |
| 1130) | | 75 | 7.25 | 0.21 |
| | | 37.5 | 3.25 | 0.21 |
| | | 18.75 | 1.20 | 0.14 |
| | | 9.38 | 0.60 | 0.14 |
| nCas9-polQ(1819-2590) | G031155 + | 300 | 2.95 | 0.21 |
| (SEQ ID NO: 1082) | G025992 | 150 | 3.35 | 0.64 |
| | | 75 | 1.95 | 0.21 |
| | | 37.5 | 0.85 | 0.07 |
| | | 18.75 | 0.40 | 0.00 |
| | | 9.38 | 0.10 | 0.14 |

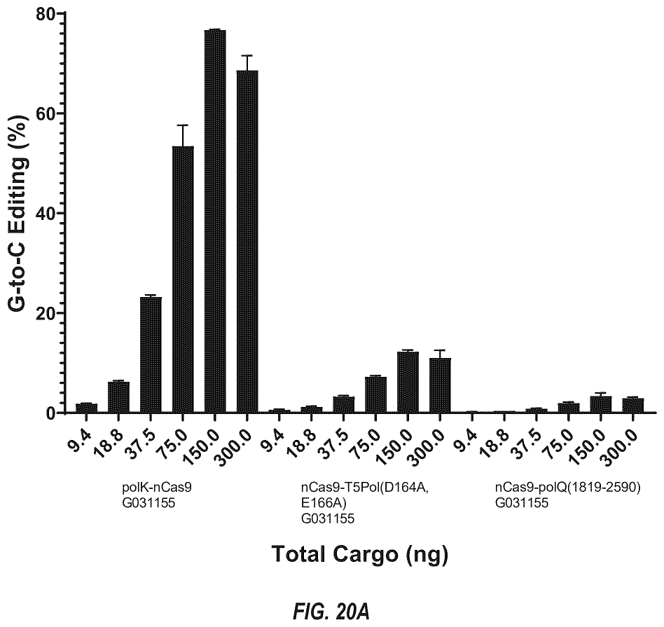

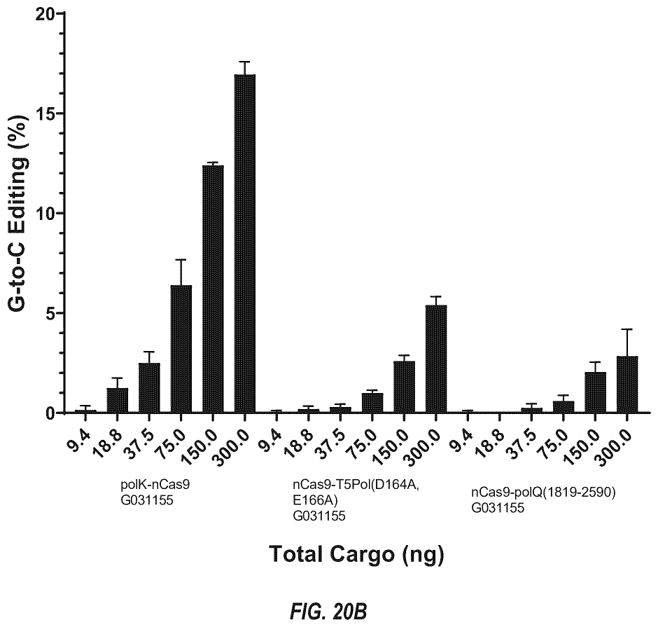

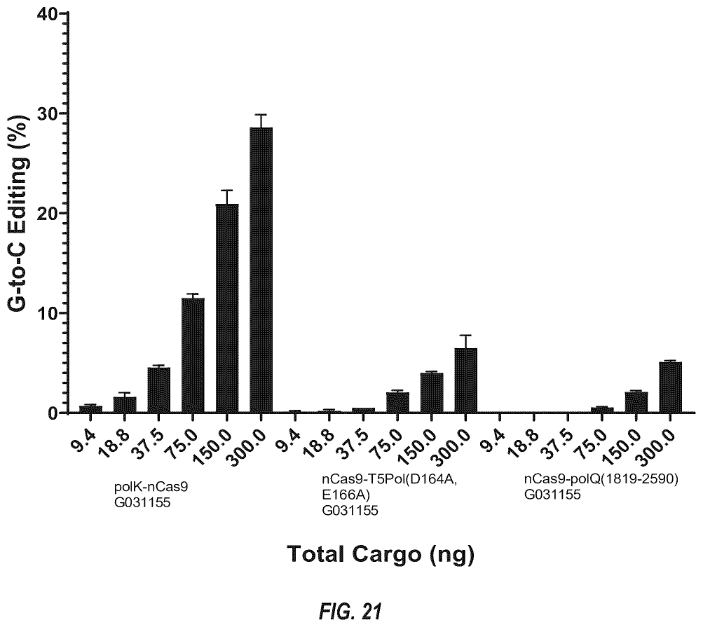

In a second experiment, the same guides and mRNAs were delivered to Huh7 cells. The cells were prepared as described in Example 1, plated at a density of 15,000 cells/well, and incubated at 37° C. and 5% CO₂ atmosphere for 24 hours. RNAs were delivered to the cells using Lipofectamine™ MessengerMAX™ Reagent (Thermo Fisher, Cat #LMRNA001) according to the manufacturer's protocol. Lipoplexes were formed, each containing either mRNA or guide RNA. Lipoplexes comprising mRNA and lipoplexes comprising guide RNA were delivered simultaneously to the cells at a ratio of 2:1 mRNA: gRNA by weight. When ngRNA (G025992) was added, the lipoplexes containing ngRNA were delivered simultaneously along with the lipoplexes containing mRNA and the lipoplexes containing tgRNA, wherein the total guide RNA (tgRNA+ngRNA) to mRNA weight ratio remained 2:1, and the tgRNA:ngRNA ratio was 3:2 by weight. Total RNA cargo dose corresponded to the combined weight of guide RNA and mRNA species. Total cargo was serially diluted by 2-fold for a 6-point dilution series. Samples were run in duplicates. Cells were lysed 72 hours post-treatment for NGS analysis as described in Example 1. Mean percent templated G-to-C transversions with standard deviation (SD) are shown in Table 23 and FIGS. 20B and 21 below.

TABLE 23
Templated editing at the HEK3 locus in Huh7 cells with
MessengerMax ™ delivery of RNA species.

| MessengerMax ™ Delivery | | | G-to-C | |
|---|---|---|---|---|
| | Guide | Cargo Dose | Editing (%) | |
| mRNA ID | RNA | (ng RNA) | Mean | SD |
| polK-nCas9 (SEQ ID NO: | G031155 | 300 | 16.95 | 0.64 |
| 1025) | | 150 | 12.40 | 0.14 |
| | | 75 | 6.40 | 1.27 |
| | | 37.5 | 2.50 | 0.57 |
| | | 18.75 | 1.25 | 0.49 |
| | | 9.38 | 0.15 | 0.21 |
| nCas9- | G031155 | 300 | 5.40 | 0.42 |
| T5Pol(D164A, E166A) | | 150 | 2.60 | 0.28 |
| (SEQ ID NO: 1130) | | 75 | 1.00 | 0.14 |
| | | 37.5 | 0.30 | 0.14 |
| | | 18.75 | 0.20 | 0.14 |
| | | 9.38 | 0.05 | 0.07 |
| nCas9-polQ(1819-2590) | G031155 | 300 | 2.85 | 1.34 |
| (SEQ ID NO: 1082) | | 150 | 2.05 | 0.49 |
| | | 75 | 0.60 | 0.28 |
| | | 37.5 | 0.25 | 0.21 |
| | | 18.75 | 0.00 | 0.00 |
| | | 9.38 | 0.05 | 0.07 |
| polK-nCas9 (SEQ ID NO: | G031155 + | 300 | 28.60 | 1.27 |
| 1025) | G025992 | 150 | 20.95 | 1.34 |
| | | 75 | 11.50 | 0.42 |
| | | 37.5 | 4.55 | 0.21 |
| | | 18.75 | 1.60 | 0.42 |
| | | 9.38 | 0.70 | 0.14 |
| nCas9- | G031155 + | 300 | 6.50 | 1.27 |
| T5Pol(D164A, E166A) | G025992 | 150 | 4.00 | 0.14 |
| (SEQ ID NO: 1130) | | 75 | 2.05 | 0.21 |
| | | 37.5 | 0.50 | 0.00 |
| | | 18.75 | 0.20 | 0.14 |
| | | 9.38 | 0.15 | 0.07 |
| nCas9-polQ(1819-2590) | G031155 + | 300 | 5.10 | 0.14 |
| (SEQ ID NO: 1082) | G025992 | 150 | 2.10 | 0.14 |
| | | 75 | 0.55 | 0.07 |
| | | 37.5 | 0.10 | 0.00 |
| | | 18.75 | 0.10 | 0.00 |
| | | 9.38 | 0.00 | 0.00 |

### Example 8—Template Based Editing in Huh7 Cells with Template Guides with Various DRS and Template Lengths

This Example evaluated templated editing efficiency with tgRNAs having different DNA polymerase recruiting sequence (DRS) lengths or template lengths. Six different tgRNAs were assessed with a DRS length of either 10 or 12 nucleotides and a template length of either 11, 13, or 15 nucleotides. See Table 24 for a description of the guide RNAs evaluated in this Example.

Further, the tgRNAs were evaluated in combination with single polypeptide fusion proteins including a SpyCas9 nickase operably linked to different DNA-dependent DNA polymerases (PolK or T5). In particular, the following single polypeptide fusions were evaluated polk-nCas9 (SEQ ID NO: 1025) and nCas9-T5Pol(D164A,E166A (SEQ ID NO: 1130). Each Cas9/DNA polymerase fusion was complexed with a tgRNA and assessed for in vitro template-based editing.

Template guide RNAs (tgRNAs) comprising the same 20-nucleotide spacer sequence targeting the HEK3 genomic locus, were designed with chimeric RNA/DNA 3′ extensions encoding a G-to-C transversion and that varied in the length of the DRS or template region, as listed in Table 24. An additional ngRNA (G025992), which targets the opposite strand of the DNA duplex target nucleic acid from the tgRNA, 90 bp away from the cut site, was used. Guides were delivered into human hepatoma (Huh7) cells along with mRNAs encoding a fusion enzyme selected from polK-nCas9 (pol Kappa moiety fused to a SpyCas9 H840A nickase in N-to-C orientation) (SEQ ID NO: 1025) and nCas9-T5Pol(D164A,E166A) (a SpyCas9 H840A nickase fused to a T5 Pol comprising mutations D164A and E166A in N-to-C orientation) (SEQ ID NO: 1130). Editing efficiency was evaluated by NGS.

TABLE 24
DRS and template sequence lengths in different tgRNAs.

| Guide ID | DRS (nt) | Template (nt) |
|---|---|---|
| G031154 (SEQ ID NO: 480) | 10 | 15 |
| G031155 (SEQ ID NO: 503) | | 13 |
| G031156 (SEQ ID NO: 504) | | 11 |
| G031157 (SEQ ID NO: 505) | 12 | 15 |
| G031158 (SEQ ID NO: 506) | | 13 |
| G031159 (SEQ ID NO: 507) | | 11 |

Cells were prepared as described in Example 1, plated at a density of 15,000 cells/well and incubated at 37° C. and 5% CO₂ atmosphere for 24 hours.

LNPs were prepared as described in Example 1, with a molar ratio of 50% Lipid A, 38% cholesterol, 9% DSPC, and 3% PEG2k-DMG, and a lipid amine to RNA phosphate (N: P) molar ratio of about 6.

Each LNP contained a single RNA cargo. LNP containing mRNA and LNP containing guide RNA were delivered simultaneously at a ratio of 2:1 by RNA weight, respectively. Total RNA cargo dose corresponded to the combined weight of guide RNA and mRNA. Total cargo was serially diluted by 2-fold for a 6-point dilution series. LNPs were diluted in cell culture media as prepared above with additional 10% FBS, incubated at 37° C. for 10 minutes, and then added to the cells. All conditions were carried out in duplicates.

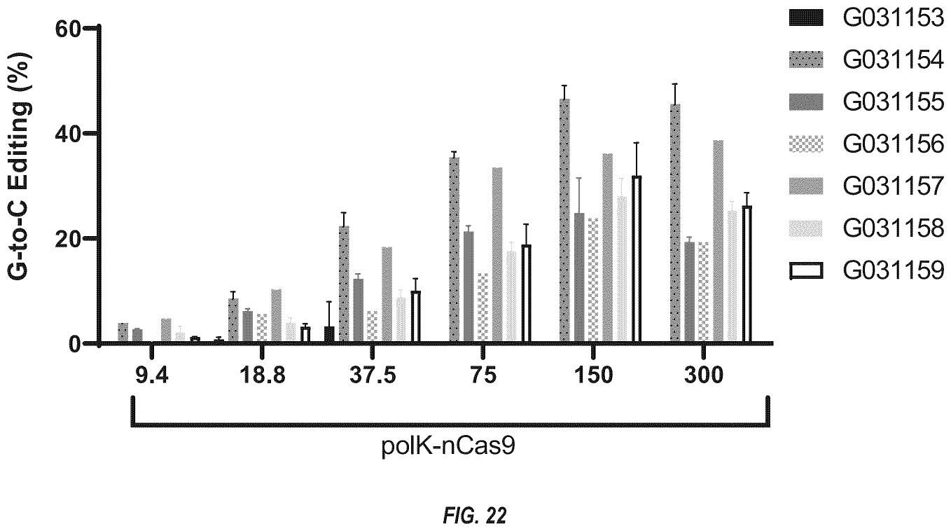

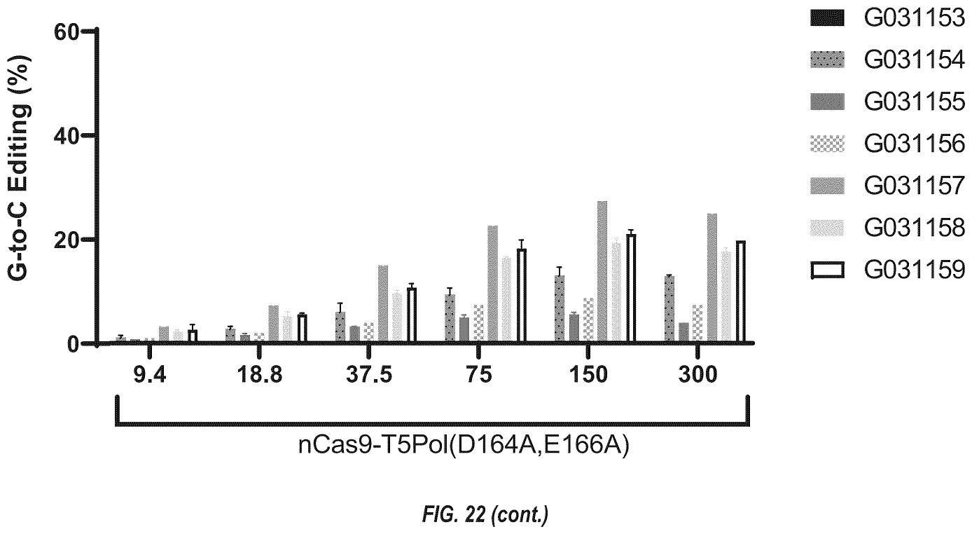

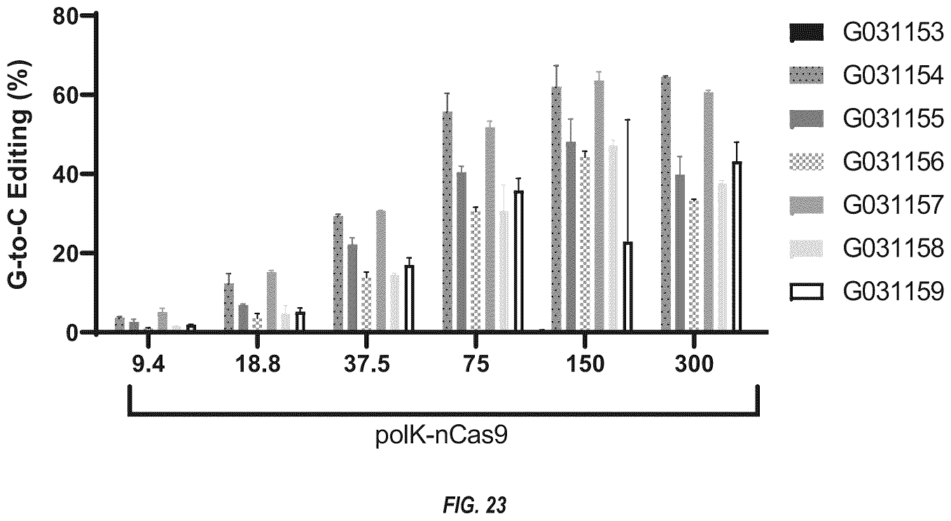

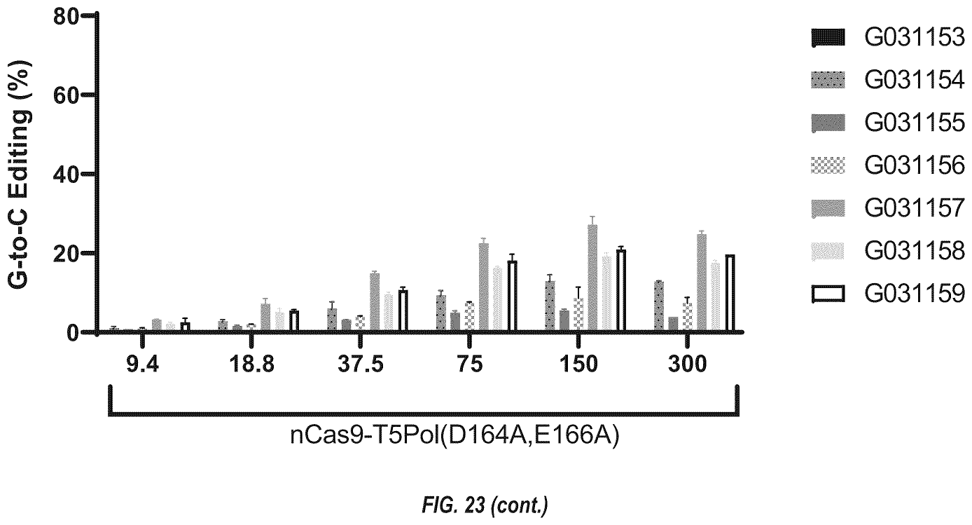

Cells were lysed 72 hours post-treatment for NGS analysis as described in Example 1. Mean percent templated G-to-C transversions with standard deviation (SD) are shown in Tables 25 and 26 and FIGS. 22 and 23 below.

TABLE 25
Templated G-to-C editing at the HEK3 locus in the absence of ngRNA, with DNA dependent
polymerases using guides containing different DRS and template lengths

| | | G031153 | | | | | | | | | | | | | |
|---|---|---|---|---|---|---|---|---|---|---|---|---|---|---|---|
| | | (SEQ ID NO: 479) | | G031154 | | G031155 | | G031156 | | G031157 | | G031158 | | G031159 | |
| | | Mean | | Mean | | Mean | | Mean | | Mean | | Mean | | Mean | |
| | Total | G-to-C | | G-to-C | | G-to-C | | G-to-C | | G-to-C | | G-to-C | | G-to-C | |
| | Cargo | Editing | | Editing | | Editing | | Editing | | Editing | | Editing | | Editing | |
| mRNA ID | (ng) | (%) | SD | (%) | SD | (%) | SD | (%) | SD | (%) | SD | (%) | SD | (%) | SD |
| polK- | 300 | 0.10* | N/A | 45.60 | 3.82 | 19.30 | 0.99 | 19.30 | 1.41 | 38.70 | 4.10 | 25.30 | 1.84 | 26.25 | 2.47 |
| nCas9 | 150 | 0.25 | 0.07 | 46.60 | 2.55 | 24.90 | 6.65 | 23.85 | 4.31 | 36.20 | 7.35 | 28.05 | 3.46 | 31.95 | 6.29 |
| (SEQ ID | 75 | 0.20* | N/A | 35.45 | 1.06 | 21.30 | 1.13 | 13.40 | 0.71 | 33.50 | 3.68 | 17.55 | 1.77 | 18.90 | 3.82 |
| NO: 1025) | 37.5 | 3.30 | 4.67 | 22.40 | 2.55 | 12.35 | 0.92 | 6.20 | 1.70 | 18.35 | 1.91 | 8.75 | 1.48 | 10.10 | 2.26 |
| | 18.75 | 0.75 | 0.49 | 8.55 | 1.34 | 6.20 | 0.42 | 5.65 | 3.89 | 10.35 | 0.07 | 4.00 | 0.99 | 3.20 | 0.57 |
| | 9.38 | 0.00 | 0.00 | 3.90 | 0.00 | 2.70 | 0.14 | 0.45 | 0.64 | 4.75 | 2.05 | 2.10 | 1.27 | 1.25 | 0.07 |
| nCas9- | 300 | 0.00 | 0.00 | 12.85 | 0.21 | 3.90 | 0.00 | 7.35 | 1.48 | 24.85 | 0.78 | 17.60 | 0.71 | 19.70 | 0.00 |
| T5Pol | 150 | 0.20 | 0.14 | 13.00 | 1.56 | 5.55 | 0.35 | 8.65 | 2.76 | 27.25 | 2.05 | 19.30 | 0.85 | 20.95 | 0.78 |
| (D164A, | 75 | 0.00 | 0.00 | 9.35 | 1.20 | 4.95 | 0.49 | 7.35 | 0.35 | 22.55 | 1.20 | 16.40 | 0.28 | 18.15 | 1.63 |
| E166A) | 37.5 | 0.00 | 0.00 | 6.00 | 1.70 | 3.15 | 0.07 | 3.90 | 0.28 | 14.95 | 0.49 | 9.60 | 0.57 | 10.70 | 0.71 |
| (SEQ ID | 18.75 | 0.00 | 0.00 | 2.80 | 0.42 | 1.65 | 0.21 | 2.05 | 0.07 | 7.25 | 1.34 | 5.15 | 0.92 | 5.50 | 0.28 |
| NO: 1130) | 9.38 | 0.00 | 0.00 | 1.10 | 0.42 | 0.80 | 0.00 | 0.95 | 0.21 | 3.25 | 0.07 | 2.25 | 0.35 | 2.60 | 0.99 |

*Indicates an experimental group for which N = 1

TABLE 26
Templated G-to-C transversions at the HEK3 locus in the presence of ngRNA, with DNA-
dependent DNA polymerases using guides containing different DRS and template lengths

| | | G031153 | | G031154 | | G031155 | | G031156 | | G031157 | | G031158 | | G031159 | |
|---|---|---|---|---|---|---|---|---|---|---|---|---|---|---|---|
| | | Mean | | Mean | | Mean | | Mean | | Mean | | Mean | | Mean | |
| | Total | G-to-C | | G-to-C | | G-to-C | | G-to-C | | G-to-C | | G-to-C | | G-to-C | |
| mRNA | Cargo | Editing | | Editing | | Editing | | Editing | | Editing | | Editing | | Editing | |
| ID | (ng) | (%) | SD | (%) | SD | (%) | SD | (%) | SD | (%) | SD | (%) | SD | (%) | SD |
| polK- | 300 | 0.15 | 0.07 | 64.60 | 0.14 | 39.85 | 4.60 | 33.20 | 0.42 | 60.60 | 0.57 | 37.70 | 0.71 | 43.20 | 4.81 |
| nCas9 | 150 | 0.50 | 0.14 | 62.00 | 5.37 | 48.15 | 5.73 | 44.20 | 1.56 | 63.60 | 2.26 | 47.30 | 1.27 | 22.90 | 30.83 |
| (SEQ ID | 75 | 0.25 | 0.07 | 55.75 | 4.60 | 40.40 | 1.56 | 30.50 | 1.13 | 51.85 | 1.48 | 30.70 | 6.51 | 35.85 | 3.04 |
| NO: 1025) | 37.5 | 0.15 | 0.07 | 29.45 | 0.35 | 22.20 | 1.7 | 13.80 | 1.41 | 30.70 | 0.14 | 14.55 | 0.49 | 17.05 | 1.77 |
| | 18.75 | 0.05 | 0.07 | 12.40 | 2.40 | 6.90 | 0.28 | 3.55 | 1.20 | 15.25 | 0.35 | 4.75 | 2.05 | 5.20 | 0.99 |
| | 9.38 | 0.05 | 0.07 | 3.70 | 0.28 | 2.65 | 0.64 | 0.95 | 0.21 | 5.15 | 0.92 | 1.65 | 0.07 | 1.95 | 0.07 |
| nCas9- | 300 | 0.00 | 0.00 | 12.85 | 0.21 | 3.90 | 0.00 | 7.35 | 1.48 | 24.85 | 0.78 | 17.60 | 0.71 | 19.70 | 0.00 |
| T5Pol | 150 | 0.20 | 0.14 | 13.00 | 1.56 | 5.55 | 0.35 | 8.65 | 2.76 | 27.25 | 2.05 | 19.30 | 0.85 | 20.95 | 0.78 |
| (D164A, | 75 | 0.00 | 0.00 | 9.35 | 1.20 | 4.95 | 0.49 | 7.35 | 0.35 | 22.55 | 1.20 | 16.40 | 0.28 | 18.15 | 1.63 |
| E166A) | 37.5 | 0.00 | 0.00 | 6.00 | 1.70 | 3.15 | 0.07 | 3.90 | 0.28 | 14.95 | 0.49 | 9.60 | 0.57 | 10.70 | 0.71 |
| (SEQ ID | 18.75 | 0.00 | 0.00 | 2.80 | 0.42 | 1.65 | 0.21 | 2.05 | 0.07 | 7.25 | 1.34 | 5.15 | 0.92 | 5.50 | 0.28 |
| NO: 1130) | 9.38 | 0.00 | 0.00 | 1.10 | 0.42 | 0.80 | 0.00 | 0.95 | 0.21 | 3.25 | 0.07 | 2.25 | 0.35 | 2.60 | 0.99 |

### Example 9—Template Based Editing in Huh7 Cells with Various Template Guide RNA (tgRNA):mRNA Ratios

This Example evaluated templated editing efficiency with different ratios of tgRNA to mRNA (i.e., mRNA encoding a SpyCas9 nickase fused to a DNA polymerase). Further, the tgRNAs were evaluated in combination with single polypeptide fusion proteins including a SpyCas9 nickase operably linked with different DNA-dependent DNA polymerases (PolK or T5).

In particular, the following single polypeptide fusions were evaluated: polk-nCas9 (SEQ ID NO: 1025) and nCas9-T5Pol(D164A,E166A (SEQ ID NO: 1130). Each Cas9/DNA polymerase fusion was complexed with a tgRNA and assessed for in vitro template-based editing. See Table 27 for a description of the guide RNAs evaluated in this Example.

Two template guide RNAs (tgRNAs), comprising the same 20-nucleotide spacer sequence targeting the HEK3 genomic locus, were designed with either an RNA 3′ extension or a chimeric RNA/DNA 3′ extension with varying DRS and template lengths as listed in Table 27. These guides were paired with mRNAs encoding a SpyCas9 H840A nickase fused to a DNA polymerase, at different mRNA: gRNA ratios. Guides and mRNAs were delivered into human hepatoma (Huh7) cells and editing efficiency was evaluated by NGS.

TABLE 27
DRS and template sequence lengths in different tgRNAs.

| Guide ID | DRS (nt) | Template (nt) |
|---|---|---|
| G031154 | 10 | 15 |
| G031157 | 12 | 15 |

Cells were prepared as described in Example 1, plated at a density of 15,000 cells/well and incubated at 37° C. and 5% CO₂ atmosphere for 24 hours.

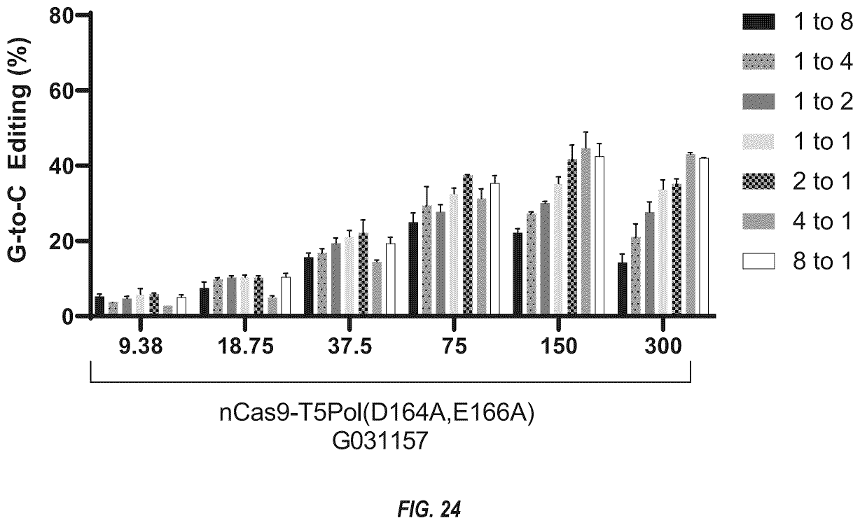

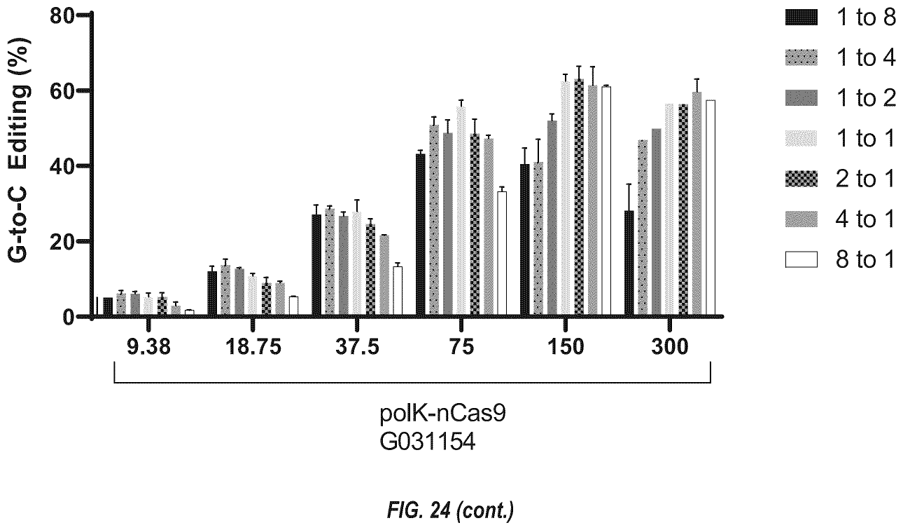

LNPs were prepared as described in Example 1, with a molar ratio of 50% Lipid A, 38% cholesterol, 9% DSPC, and 3% PEG2k-DMG, and a lipid amine to RNA phosphate (N:P) molar ratio of about 6. Each LNP contained a single RNA cargo. LNP containing mRNA and LNP containing guide RNA were delivered simultaneously at varying ratios of mRNA to guide RNA, as indicated in Table 28 and FIG. 24. Total RNA cargo dose corresponded to the combined weight of guide RNA and mRNA. Total RNA cargo was serially diluted 2-fold for a 6-point dilution series. LNPs were diluted in cell culture media with additional 10% FBS, incubated at 37° C. for 10 minutes and then added to cells. All conditions were carried out in duplicates.

Cells were lysed 4 days post-treatment for NGS analysis as described in Example 1. Mean templated G-to-C transversion percentage with standard deviation (SD) are indicated in Table 28 and FIG. 24 below.

TABLE 28
Percentage of templated G-to-C editing achieved at the HEK3 locus with various ratios of mRNA to tgRNA

| Total RNA | | | Ratio of mRNA to tgRNA by weight | | | | | | | | | | | | | |
|---|---|---|---|---|---|---|---|---|---|---|---|---|---|---|---|---|
| Cargo | mRNA | tgRNA | 1:8 | | 1:4 | | 1:2 | | 1:1 | | 2:1 | | 4:1 | | 8:1 | |
| Dose (ng) | ID | ID | Mean | SD | Mean | SD | Mean | SD | Mean | SD | Mean | SD | Mean | SD | Mean | SD |
| 300 | nCas9- | G031157 | 14.30 | 2.26 | 21.05 | 3.46 | 27.65 | 2.76 | 33.75 | 2.47 | 35.20 | 1.27 | 43.10 | 0.42 | 42.00 | 0.14 |
| 150 | T5Pol | | 22.10 | 1.13 | 27.30 | 0.42 | 30.05 | 0.49 | 35.25 | 1.77 | 41.75 | 3.75 | 44.70 | 4.24 | 42.50 | 3.39 |
| 75 | (D164A, | | 24.95 | 2.47 | 29.45 | 5.02 | 27.75 | 1.91 | 32.55 | 1.48 | 37.50 | 0.14 | 31.30 | 2.55 | 35.35 | 2.05 |
| 37.50 | E166A) | | 15.70 | 1.13 | 16.85 | 1.06 | 19.40 | 1.41 | 21.10 | 1.70 | 22.20 | 3.39 | 14.50 | 0.42 | 19.35 | 1.63 |
| 18.75 | (SEQ ID | | 7.50 | 1.56 | 9.85 | 0.35 | 10.30 | 0.42 | 10.40 | 0.57 | 10.25 | 0.49 | 4.95 | 0.49 | 10.40 | 0.99 |
| 9.38 | NO: 1130) | | 5.25 | 0.64 | 3.80 | 0.00 | 4.70 | 0.57 | 5.80 | 1.56 | 5.90 | 0.28 | 2.80 | 0.00 | 5.05 | 0.64 |
| 300 | polK- | G031154 | 28.15 | 7.00 | 46.90 | N/A | 49.90* | N/A | 56.70* | N/A | 56.40* | N/A | 59.70 | 3.39 | 57.50* | N/A |
| 150 | nCas9 | | 40.50 | 4.24 | 41.00 | 6.08 | 52.00 | 1.84 | 62.55 | 1.77 | 63.15 | 3.32 | 61.40 | 4.95 | 61.05 | 0.35 |
| 75 | (SEQ ID | | 43.20 | 0.99 | 50.95 | 2.05 | 48.75 | 3.46 | 55.85 | 1.63 | 48.55 | 3.89 | 47.30 | 0.85 | 33.25 | 1.20 |
| 37.50 | NO: 1025) | | 27.15 | 2.47 | 28.75 | 0.64 | 26.75 | 1.06 | 27.90 | 3.11 | 24.60 | 1.41 | 21.60 | 0.14 | 13.35 | 0.92 |
| 18.75 | | | 12.00 | 1.41 | 13.65 | 1.63 | 12.70 | 0.28 | 10.80 | 0.71 | 8.95 | 1.48 | 8.95 | 0.49 | 5.35 | 0.07 |
| 9.38 | | | 5.00* | N/A | 6.15 | 0.78 | 6.10 | 0.57 | 5.30 | 0.99 | 5.20 | 1.13 | 2.90 | 0.99 | 1.75 | 0.07 |

*Indicates an experimental group for which N = 1

### Example 10—Template Based Editing in Huh7 Cells with T5 DNA Polymerase Variants

This Example evaluated templated editing efficiency with different T5 DNA Polymerases. In particular, this study assessed single polypeptide fusions including a SpyCas9 nickase (nCas9) operably linked to T5 DNA polymerases having various amino acid substitutions (e.g., selected from two or more of D164A, E166A, N30del, C24del, C63del, K425I, or A593R). Further assessed were (i) single polypeptide fusions with different C- to N-terminus arrangements; (ii) a single polypeptide fusion having heterologous nuclear localization signals (e.g., SV40 and cMycNLS); and (iv) a single polypeptide fusion having an MS2 coat protein (MCP). Each Cas9/DNA polymerase fusion was complexed with a tgRNA and assessed for in vitro template-based editing.

The following single polypeptide fusions were evaluated in this Example:
(i) nCas9-T5Pol(D164A,E166A) (SEQ ID NO: 1130)
(ii) T5Pol(D164A,E166A)-nCas9 (SEQ ID NO: 1125)
(iii) nCas9-T5Pol(D164A,E166A)-bpSV40-CMycNLS (SEQ ID NO: 1138)
(iv) nCas9-T5Pol(D164A,E166A)-MCP (SEQ ID NO: 1168)
(v) nCas9-T5Pol(N30del,D164A,E166A) (SEQ ID NO: 1148);
(vi) nCas9-T5Pol (C24del,D164A,E166A) (SEQ ID NO: 1153)
(vii) nCas9-T5Pol (C63del,D164A,E166A) (SEQ ID NO: 1158)
(viii) nCas9-T5Pol(D164A,E166A,K425I) (SEQ ID NO: 1163); and
(ix) nCas9-T5Pol(D164A,E166A,A593R) (SEQ ID NO: 1143).

Further, six different tgRNAs were assessed with a DNA polymerase recruiting sequence (DRS) length of either 10 or 12 nucleotides and a template length of either 11, 13, or 15 nucleotides. See Table 29 for a description of the guide RNAs evaluated in this Example.

Template guide RNAs (tgRNAs) comprising the same 20-nucleotide spacer sequence targeting the HEK3 genomic locus, were designed with chimeric RNA/DNA 3′ extensions that varied in the length of the DRS or template region, as listed in Table 29.

TABLE 29
DRS and template sequence lengths in different tgRNAs.

| Guide ID | DRS (nt) | Template (nt) |
|---|---|---|
| G031154 | 10 | 15 |
| G031155 | | 13 |
| G031156 | | 11 |
| G031157 | 12 | 15 |
| G031158 | | 13 |
| G031159 | | 11 |

These guides were delivered into human hepatoma (Huh7) cells along with mRNAs encoding a H840A SpyCas9 nickase fused to different variants of T5 polymerase, referred to herein as T5 variant moieties. An additional ngRNA (G025992), which targets the opposite strand of the DNA duplex target nucleic acid from the tgRNA 90 bp away from the cut site, was used. Editing efficiency was evaluated by NGS as described in Example 1.

Cells were prepared as described in Example 1, plated at a density of 15,000 cells/well and incubated at 37° C. and 5% CO₂ atmosphere for 24 hours.

RNAs were delivered to the cells using Lipofectamine™ MessengerMAX™ Reagent (Thermo Fisher, Cat. LMRNA001) according to the manufacturer's protocol. Lipoplexes were formed, each containing a single species of either mRNA or guide RNA, and then were delivered simultaneously to the cells at a ratio of 2:1 mRNA: gRNA by weight.

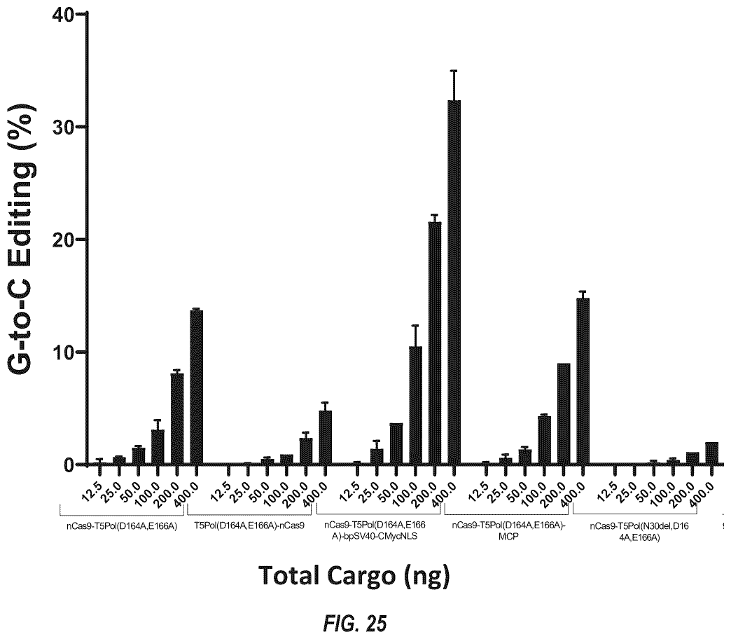

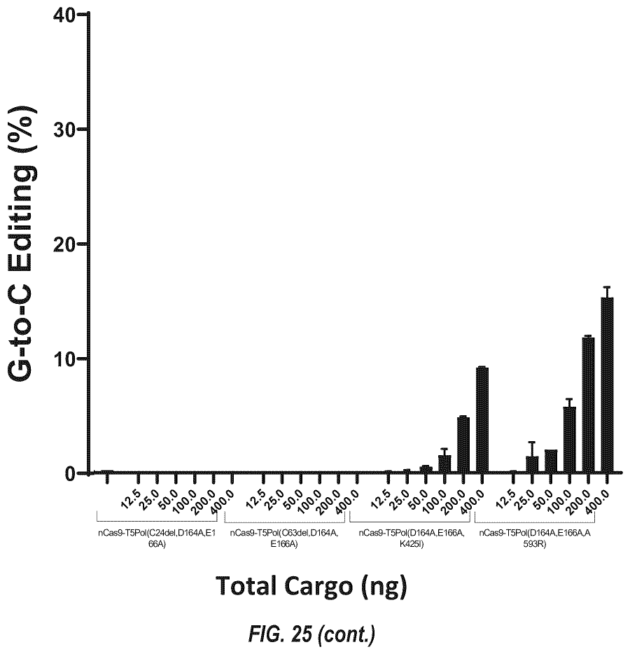

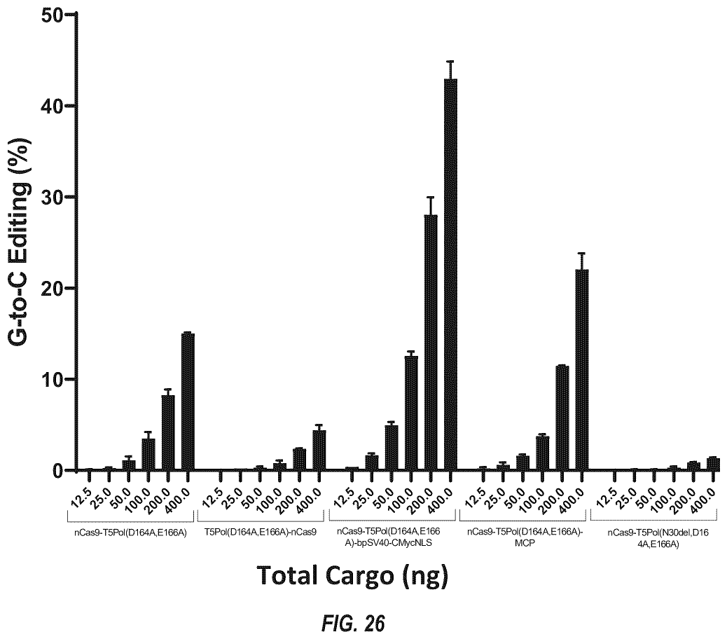

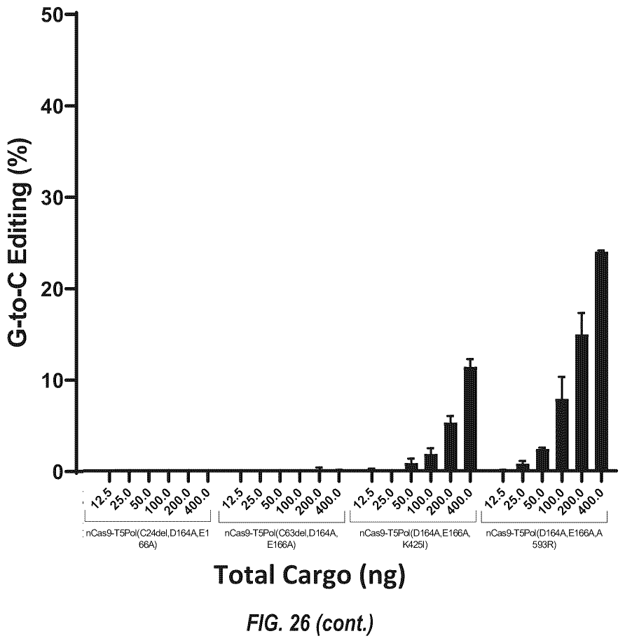

Two separate experiments were conducted. In the first experiment, mRNAs were delivered with G031157 with or without the addition of ngRNA (G025992), and total RNA cargo was serially diluted 2-fold for a 6-point dilution series. When adding ngRNA, the guide RNA amount comprised both tgRNA and ngRNA and the molar ratio of tgRNA:ngRNA was 3:2. Samples were run in duplicates. Cells were lysed 72 hours post-treatment for NGS analysis as described in Example 1. Mean percent templated G-to-C transversions, with standard deviation (SD) for this experiment are shown in Tables 30 and 31 and FIGS. 25 and 26.

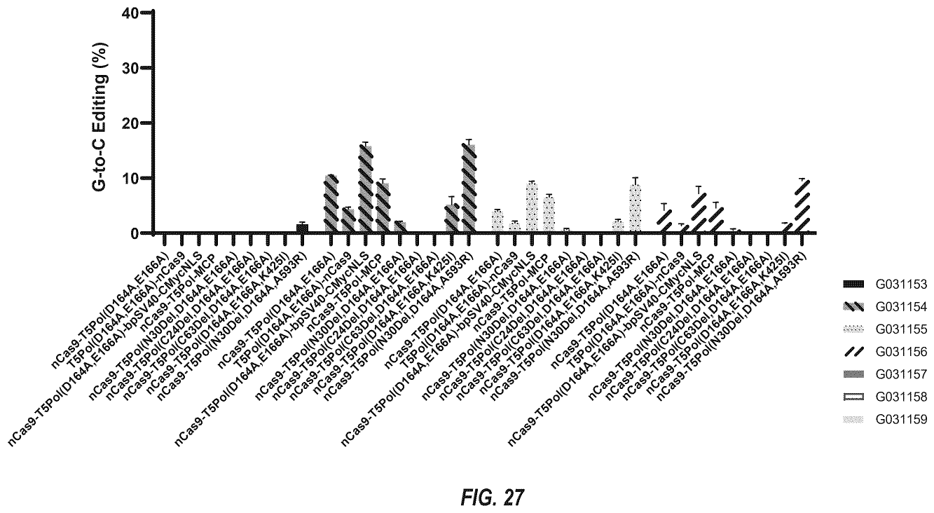

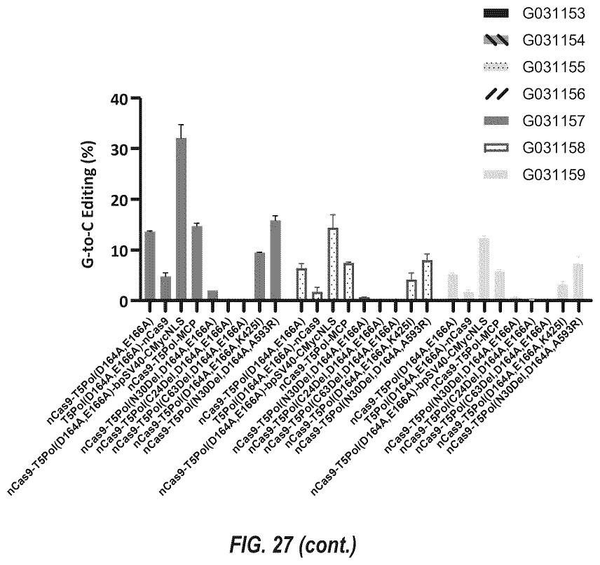

In the second experiment, each mRNA was delivered with each of the guides listed in Table 29 at a single dose of 400 ng total RNA cargo. Samples were run in duplicates. Cells were lysed 72 hours post-treatment for NGS analysis as described in Example 1. Mean percent templated G-to-C transversions with standard deviation (SD) for this experiment are shown in Table 32 and FIG. 27.

TABLE 30
Percent templated G-to-C editing at the HEK3 locus in Huh7 cells using G031157
and nCas9-DNA polymerase fusion proteins with T5 variant moieties

| | | | | | nCas9- | | | | | | | | | | | | | |
|---|---|---|---|---|---|---|---|---|---|---|---|---|---|---|---|---|---|---|
| | | | | | T5Pol | | nCas9- | | nCas9- | | nCas9- | | nCas9- | | nCas9- | | nCas9- | |
| | nCas9- | | T5Pol | | (D164A, | | T5Pol | | T5Pol, | | T5Pol | | T5Pol | | T5Pol | | T5Pol | |
| | T5Pol | | (D164A, | | E166A)- | | (D164A, | | (N30del | | (C24del, | | (C63del, | | (D164A, | | (D164A, | |
| | (D164A, | | E166A)- | | bpSV40- | | E166A)- | | D164A, | | D164A, | | D164A, | | E166A, | | E166A, | |
| mRNA | E166A) | | nCas9 | | CMycNLS | | MCP | | E166A) | | E166A) | | E166A) | | K425I) | | A593R) | |
| ID | (SEQ | | (SEQ | | (SEQ | | (SEQ | | (SEQ | | (SEQ | | (SEQ | | (SEQ | | (SEQ | |
| Total | ID NO: | | ID NO: | | ID NO: | | ID NO: | | ID NO: | | ID NO: | | ID NO: | | ID NO: | | ID NO: | |
| Cargo | 1130) | | 1125) | | 1138) | | 1168) | | 1148) | | 1153) | | 1158) | | 1163) | | 1143) | |
| (ng) | Mean | SD | Mean | SD | Mean | SD | Mean | SD | Mean | SD | Mean | SD | Mean | SD | Mean | SD | Mean | SD |
| 400 | 13.7 | 0.1 | 4.8 | 0.7 | 32.4 | 2.6 | 14.8 | 0.6 | 2.0 | 0.0 | 0.0 | 0.0 | 0.0 | 0.0 | 9.6 | 0.1 | 16.0 | 0.9 |
| 200 | 8.1 | 0.3 | 2.4 | 0.5 | 21.6 | 0.6 | 9.0* | N/A | 1.1 | 0.0 | 0.0 | 0.0 | 0.0 | 0.0 | 5.1 | 0.1 | 12.3 | 0.1 |
| 100 | 3.1 | 0.9 | 0.9 | 0.0 | 10.5 | 1.8 | 4.3 | 0.1 | 0.4 | 0.1 | 0.0 | 0.0 | 0.0 | 0.0 | 1.6 | 0.6 | 6.0 | 0.7 |
| 50 | 1.5 | 0.1 | 0.5 | 0.1 | 3.7 | 0.0 | 1.4 | 0.2 | 0.2 | 0.1 | 0.0 | 0.0 | 0.0 | 0.0 | 0.6 | 0.1 | 2.1* | N/A |
| 25 | 0.7 | 0.1 | 0.1 | 0.1 | 1.4 | 0.7 | 0.6 | 0.3 | 0.0 | 0.0 | 0.0 | 0.0 | 0.1* | N/A | 0.1 | 0.1 | 1.5 | 1.3 |
| 12.5 | 0.2 | 0.3 | 0.1 | 0.0 | 0.2 | 0.1 | 0.2 | 0.1 | 0.0 | 0.0 | 0.0 | 0.0 | 0.0* | N/A | 0.1 | 0.1 | 0.1 | 0.1 |

*Indicates experimental group for which N = 1

TABLE 31
Percent templated G-to-C editing at the HEK3 locus in Huh7 cells using G031157 and nCas9-DNA
polymerase fusion proteins with T5 variant moieties in the presence of a nicking guide

| | | | | | nCas9- | | | | | | | | | | | | | |
|---|---|---|---|---|---|---|---|---|---|---|---|---|---|---|---|---|---|---|
| | | | | | T5Pol | | nCas9- | | nCas9- | | nCas9- | | nCas9- | | nCas9- | | nCas9- | |
| | nCas9- | | T5Pol | | (D164A, | | T5Pol | | T5Pol, | | T5Pol | | T5Pol | | T5Pol | | T5Pol | |
| | T5Pol | | (D164A, | | E166A)- | | (D164A, | | (N30del | | (C24del, | | (C63del, | | (D164A, | | (D164A, | |
| | (D164A, | | E166A)- | | bpSV40- | | E166A)- | | D164A, | | D164A, | | D164A, | | E166A, | | E166A, | |
| mRNA | E166A) | | nCas9 | | CMycNLS | | MCP | | E166A) | | E166A) | | E166A) | | K425I) | | A593R) | |
| ID | (SEQ | | (SEQ | | (SEQ | | (SEQ | | (SEQ | | (SEQ | | (SEQ | | (SEQ | | (SEQ | |
| Total | ID NO: | | ID NO: | | ID NO: | | ID NO: | | ID NO: | | ID NO: | | ID NO: | | ID NO: | | ID NO: | |
| Cargo | 1130) | | 1125) | | 1138) | | 1168) | | 1148) | | 1153) | | 1158) | | 1163) | | 1143) | |
| (ng) | Mean | SD | Mean | SD | Mean | SD | Mean | SD | Mean | SD | Mean | SD | Mean | SD | Mean | SD | Mean | SD |
| 400 | 15.0 | 0.1 | 4.4 | 0.6 | 43.0 | 1.9 | 22.1 | 1.8 | 1.4 | 0.1 | 0.0 | 0.0 | 0.1 | 0.1 | 11.4 | 0.9 | 24.0 | 0.1 |
| 200 | 8.3 | 0.6 | 2.4 | 0.1 | 28.1 | 1.9 | 11.5 | 0.1 | 0.9 | 0.1 | 0.0 | 0.0 | 0.2 | 0.2 | 5.3 | 0.7 | 15.0 | 2.3 |
| 100 | 3.5 | 0.7 | 0.8 | 0.3 | 12.6 | 0.5 | 3.8 | 0.2 | 0.3 | 0.1 | 0.0 | 0.0 | 0.0* | N/A | 1.9 | 0.6 | 7.9 | 2.4 |
| 50 | 1.1 | 0.4 | 0.3 | 0.1 | 5.0 | 0.4 | 1.6 | 0.1 | 0.1 | 0.1 | 0.0 | 0.0 | 0.0* | N/A | 0.9 | 0.5 | 2.4 | 0.1 |
| 25 | 0.3 | 0.1 | 0.2 | 0.0 | 1.7 | 0.2 | 0.6 | 0.3 | 0.1 | 0.1 | 0.0 | 0.0 | 0.0 | 0.0 | 0.1 | 0.0 | 0.8 | 0.3 |
| 12.5 | 0.1 | 0.1 | 0.1 | 0.0 | 0.4 | 0.0 | 0.2 | 0.1 | 0.0 | 0.0 | 0.0 | 0.0 | 0.0 | 0.0 | 0.1 | 0.1 | 0.1 | 0.1 |

*indicates experimental group for which N = 1

TABLE 32
Percent templated G-to-C editing at the HEK3 locus obtained in Huh7 cells using nCas9-DNA polymerase fusion
proteins comprising T5 variant moieties with guide RNAs comprising different template and DRS lengths

| | G031153 | | G031154 | | G031155 | | G031156 | | G031157 | | G031158 | | G031159 | |
|---|---|---|---|---|---|---|---|---|---|---|---|---|---|---|
| mRNA ID | Mean | SD | Mean | SD | Mean | SD | Mean | SD | Mean | SD | Mean | SD | Mean | SD |
| nCas9-T5Pol (D164A, E166A) | 0.0* | N/A | 10.4 | 0.1 | 4.0 | 0.3 | 4.2 | 1.2 | 13.7 | 0.1 | 6.5 | 0.9 | 5.3 | 0.4 |
| (SEQ ID NO: 1130) | | | | | | | | | | | | | | |
| T5Pol (D164A, E166A)-nCas9 | 0.0 | 0.0 | 4.4 | 0.4 | 1.9 | 0.4 | 1.4 | 0.4 | 4.8 | 0.7 | 1.8 | 0.9 | 1.8 | 0.4 |
| (SEQ ID NO: 1125) | | | | | | | | | | | | | | |
| nCas9-T5Pol (D164A, E166A)- | 0.1 | 0.1 | 15.8 | 0.7 | 9.1 | 0.4 | 7.2 | 1.3 | 32.4 | 2.6 | 14.5 | 2.6 | 12.5 | 0.5 |
| bpSV40-CMycNLS (SEQ ID | | | | | | | | | | | | | | |
| NO: 1138) | | | | | | | | | | | | | | |
| nCas9-T5Pol(D164A, E166A)- | 0.0 | 0.0 | 9.1 | 0.8 | 6.6 | 0.5 | 4.6 | 1.1 | 14.8 | 0.6 | 7.5 | 0.2 | 5.9 | 0.4 |
| MCP (SEQ ID NO: 1168) | | | | | | | | | | | | | | |
| nCas9-T5Pol(N30del, D164A, | 0.0* | N/A | 2.0 | 0.2 | 0.7 | 0.2 | 0.5 | 0.3 | 2.0 | 0.0 | 0.7 | 0.1 | 0.7 | 0.2 |
| E166A) (SEQ ID NO: 1148) | | | | | | | | | | | | | | |
| nCas9-T5Pol(C24del, D164A, | 0.0 | 0.0 | 0.1 | 0.1 | 0.2 | 0.1 | 0.1 | 0.1 | 0.0 | 0.0 | 0.1 | 0.0 | 0.1 | 0.1 |
| E166A) (SEQ ID NO: 1153) | | | | | | | | | | | | | | |
| nCas9-T5Pol(C63del, D164A, | 0.0 | 0.0 | 0.0 | 0.0 | 0.2 | 0.1 | 0.0 | 0.0 | 0.0 | 0.0 | 0.1 | 0.0 | 0.0 | 0.0 |
| E166A)(SEQ ID NO: 1158) | | | | | | | | | | | | | | |
| nCas9-T5Pol(D164A, E166A, | 0.0 | 0.0 | 5.2 | 1.5 | 2.2 | 0.4 | 1.7 | 0.1 | 9.6 | 0.1 | 4.2 | 1.3 | 3.3 | 0.6 |
| K425I) (SEQ ID NO: 1163) | | | | | | | | | | | | | | |
| nCas9-T5Pol(D164A, E166A, | 1.6 | 0.4 | 16.1 | 0.9 | 8.8 | 1.3 | 9.5 | 0.4 | 16.0 | 0.9 | 8.1 | 1.2 | 7.4 | 1.4 |
| A593R) (SEQ ID NO: 1143) | | | | | | | | | | | | | | |

*indicates experimental group for which N = 1

### Example 11—Polymerase Screening for Template-Based Editing in Huh7 Cells with Various Polymerases (DNA Polymerase Nu, DNA Polymerase K, and DNA Polymerase θ; Including Variants)

In this Example, various DNA polymerases fused to a H840A nicking SpyCas9 nickase (nCas9) were screened for template-based editing in conjunction with an array of synthetic nucleic acid guides containing chimeric DNA/RNA 3′ extensions in Huh7 cells. The DNA polymerases assessed in this example were PolN, PolK, PolK (19-526), EcAPolI, T5, and Phi29Pol.

The DNA polymerase-Cas9 fusion differed in the DNA polymerase moieties used and the orientation of the protein moieties (i.e., nCas9-DNA polymerase fusions where the DNA polymerase moiety is C-terminal to the nCas9 moiety versus DNA polymerase-nCas9 fusions where the DNA polymerase moiety is C-terminal to the nCas9 moiety). In particular, the following DNA polymerases were assessed in the context of the DNA-polymerase-nCas9 fusions: polN (e.g., nCas9-polN; SEQ ID NO: 1095); polK (e.g., nCas9-polK, SEQ ID NO: 1022; or polK-nCas9, SEQ ID NO: 1025); polK(19-526) (e.g., nCas9-polK(19-526); SEQ ID NO: 1037); EcoPolI (e.g., nCas9-EcoPolI (SEQ ID NO: 1100)); T5 (e.g., nCas9-T5Pol (SEQ ID NO: 1135)); or Phi29Pol (e.g., nCas9-Phi29Pol; SEQ ID NO: 1193).

In vitro transcription was used to produce mRNAs encoding each of the fusion proteins as described in Example 1. Template guide RNAs included 3′ chimeric extensions differing in terms of their RNA and DNA contents as summarized in Table 33.

TABLE 33
Synthetic guide 3′ extension sequences composed of RNA and DNA bases

| Guide ID | 3′ Extension Sequence (DRS in bold) |
|---|---|
| G025991 | dTdCdTdGdCdGdAdTdCdAdCdGdTdGdCdTdCdAdGdTdT*dT*dG(C7NH2) |
| (SEQ ID | (SEQ ID NO: 912) |
| NO: 493) | |
| G029564 | dTdCdTdGdCdGdAdTdCdAdCdGdTdGdCdTdCmAmGmUmCmU*mG (SEQ ID |
| (SEQ ID | NO: 913) |
| NO: 494) | |
| G029565 | dTdCdTdGdCdGdAdTdCdAdCdGdTGCUCmAmGmUmCmU*mG (SEQ ID NO: |
| (SEQ ID | 914) |
| NO: 495) | |
| G029566 | dTdCdTdGdCdGdAdTdCdAdCdGUGCUCmAmGmUmCmU*mG (SEQ ID NO: 915) |
| (SEQ ID | |
| NO: 496) | |
| G029567 | dTdCdTdGdCdGdAdTdCdAdCGUGCUCmAmGmUmCmU*mG (SEQ ID NO: 916) |
| (SEQ ID | |
| NO: 497) | |
| G029568 | dTdCdTdGdCdGdAdTdCdACGUGCUCmAmGmUmCmU*mG (SEQ ID NO: 917) |
| (SEQ ID | |
| NO: 498) | |
| G029569 | dTdCdTdGdCdGdAdTdCACGUGCUCmAmGmUmCmU*mG (SEQ ID NO: 918) |
| (SEQ ID | |
| NO: 499) | |
| G029570 | dTdCdTdGdCdGdAdTCACGUGCUCmAmGmUmCmU*mG (SEQ ID NO: 919) |
| (SEQ ID | |
| NO: 500) | |
| G029571 | dTdCdTGdCdGdAUCACGUGCUCmAmGmUmCmU*mG (SEQ ID NO: 920) |
| (SEQ ID | |
| NO: 501) | |
| G029572 | dTdCdTdGCGAUCACGUGCUCmAmGmUmCmU*mG (SEQ ID NO: 921) |
| (SEQ ID | |
| NO: 502) | |
| G029555 | UCUGCGAUCACGUGCUmCmAmGmUmCmU*mG (SEQ ID NO: 923) |
| (SEQ ID | |
| NO: 518) | |

Note:
*denotes that the nucleotide is linked to the next nucleotide with a phosphorothioate (PS bond), a lower case “m” denotes that the nucleotide is modified with 2′-O-Me, and a lowercase “d” denotes that the nucleotide is a DNA nucleotide.

mRNA and guide components were prepared as described in Example 1 and plated at a density of 15,000 cells/well. After 24 hours of incubation at 37° C., 5% CO₂, cells were transfected with mRNAs and guides. Each guide comprised: 1) a spacer sequence targeting the HEK293 Site 3 locus (HEK3), 2) a synthetic SpyCas9 scaffold, and 3) a 3′ RNA/DNA hybrid extension encoding a G>C point mutation at the target locus.

All nucleic acid components were transfected in Huh7 cells using Lipofectamine™ MessengerMAX™ Reagent (Thermo Fisher, Cat. LMRNA001) according to manufacturer's instructions. Each transfection well contained 80 μL media, 20 μL Opti-MEM™ (Gibco, Cat. 31985088), 0.3 μL MessengerMAX™ reagent, 100 ng mRNA, and 50 nM synthetic guide RNA. The cells were lysed 72-hours post-treatment for NGS analysis as described in Example 1. Templated editing events were characterized as G>C transversion mutations at the HEK3 locus targeted by the guide RNA spacer sequence.

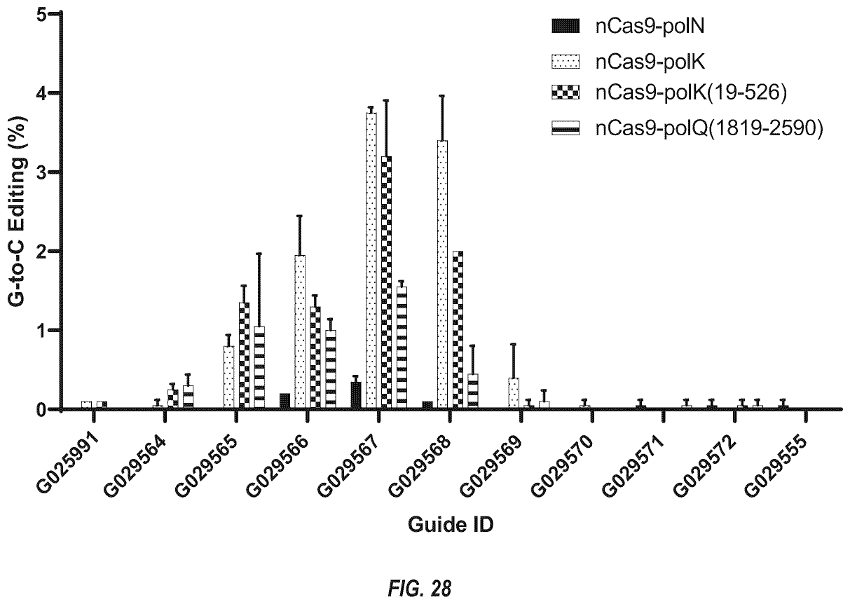

Table 34 and FIG. 28 show mean templated editing efficiency and standard deviation (N=2).

TABLE 34
Templated editing at the HEK3 locus in Huh7 cells

| mRNA ID | Guide ID | G-to-C Editing (%) | SD |
|---|---|---|---|
| nCas9-polN (SEQ ID NO: 1095) | G025991 | 0.0 | 0.0 |
| | G029564 | 0.0 | 0.0 |
| | G029565 | 0.0 | 0.0 |
| | G029566 | 0.2 | 0.0 |
| | G029567 | 0.4 | 0.1 |
| | G029568 | 0.1 | 0.0 |
| | G029569 | 0.0 | 0.0 |
| | G029570 | 0.0 | 0.0 |
| | G029571 | 0.1 | 0.1 |
| | G029572 | 0.1 | 0.1 |
| | G029555 | 0.1 | 0.1 |
| nCas9-polK (SEQ ID NO: 1022) | G025991 | 0.1 | 0.0 |
| | G029564 | 0.1 | 0.1 |
| | G029565 | 0.8 | 0.1 |
| | G029566 | 2.0 | 0.5 |
| | G029567 | 3.8 | 0.1 |
| | G029568 | 3.4 | 0.6 |
| | G029569 | 0.4 | 0.4 |
| | G029570 | 0.1 | 0.1 |
| | G029571 | 0.0 | 0.0 |
| | G029572 | 0.0 | 0.0 |
| | G029555 | 0.0 | 0.0 |
| nCas9-polK(19-526) | G025991 | 0.1 | 0.0 |
| (SEQ ID NO: 1037) | G029564 | 0.3 | 0.1 |
| | G029565 | 1.4 | 0.2 |
| | G029566 | 1.3 | 0.1 |
| | G029567 | 3.2 | 0.7 |
| | G029568 | 2.0 | 0.0 |
| | G029569 | 0.1 | 0.1 |
| | G029570 | 0.0 | 0.0 |
| | G029571 | 0.0 | 0.0 |
| | G029572 | 0.1 | 0.1 |
| | G029555 | 0.0 | 0.0 |
| nCas9-polQ(1819-2590) | G025991 | 0.0 | 0.0 |
| (SEQ ID NO: 1082) | G029564 | 0.3 | 0.1 |
| | G029565 | 1.1 | 0.9 |
| | G029566 | 1.0 | 0.1 |
| | G029567 | 1.6 | 0.1 |
| | G029568 | 0.5 | 0.4 |
| | G029569 | 0.1 | 0.1 |
| | G029570 | 0.0 | 0.0 |
| | G029571 | 0.1 | 0.1 |
| | G029572 | 0.1 | 0.1 |
| | G029555 | 0.0 | 0.0 |

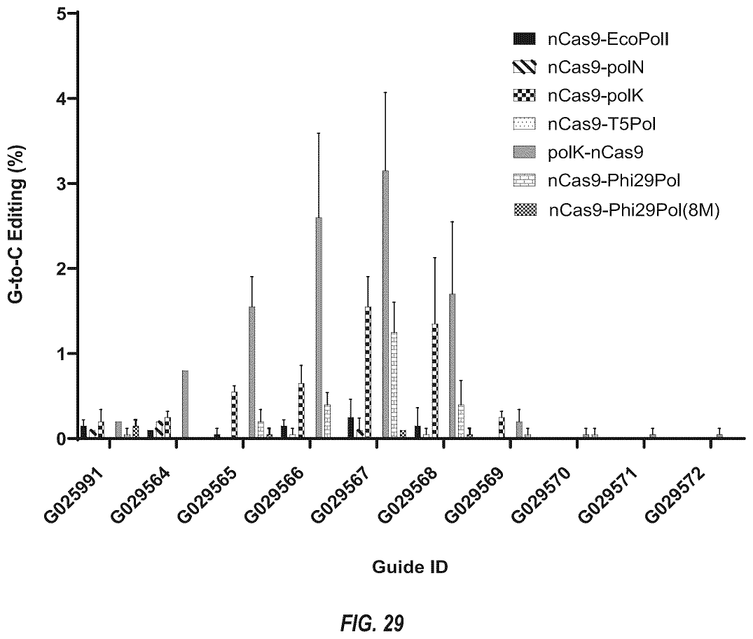

In a second experiment, another set of DNA polymerase-Cas9 fusion encoded by mRNAs was used to edit the HEK3 locus in Huh7 cells in combination with guides listed in Table 33 using the same protocol as described above. Samples were run in duplicate. Table 35 and FIG. 29 show Huh7 templated editing efficiency obtained with these fusion proteins.

TABLE 35
Templated editing at the HEK3 locus in Huh7 cells

| mRNA ID | Guide ID | G-to-C Editing (%) | SD | N |
|---|---|---|---|---|
| nCas9-EcoPolI (SEQ ID | G025991 | 0.2 | 0.1 | 2 |
| NO: 1100) | G029564 | 0.1 | 0.0 | 2 |
| | G029565 | 0.1 | 0.1 | 2 |
| | G029566 | 0.2 | 0.1 | 2 |
| | G029567 | 0.3 | 0.2 | 2 |
| | G029568 | 0.2 | 0.2 | 2 |
| | G029569 | 0.0 | 0.0 | 2 |
| | G029570 | 0.0 | 0.0 | 2 |
| | G029571 | 0.0 | 0.0 | 2 |
| | G029572 | 0.0 | 0.0 | 2 |
| nCas9-polN (SEQ ID NO: | G025991 | 0.1 | 0.0 | 2 |
| 1095) | G029564 | 0.2 | 0.0 | 2 |
| | G029565 | 0.0 | 0.0 | 2 |
| | G029566 | 0.1 | 0.1 | 2 |
| | G029567 | 0.1 | 0.1 | 2 |
| | G029568 | 0.1 | 0.1 | 2 |
| | G029569 | 0.0 | 0.0 | 2 |
| | G029570 | 0.0 | 0.0 | 2 |
| | G029571 | 0.0 | 0.0 | 2 |
| | G029572 | 0.0 | 0.0 | 2 |
| nCas9-polK (SEQ ID NO: | G025991 | 0.2 | 0.1 | 2 |
| 1022) | G029564 | 0.3 | 0.1 | 2 |
| | G029565 | 0.6 | 0.1 | 2 |
| | G029566 | 0.7 | 0.2 | 2 |
| | G029567 | 1.6 | 0.4 | 2 |
| | G029568 | 1.4 | 0.8 | 2 |
| | G029569 | 0.3 | 0.1 | 2 |
| | G029570 | 0.0 | 0.0 | 2 |
| | G029571 | 0.0 | 0.0 | 2 |
| | G029572 | 0.0 | 0.0 | 2 |
| nCas9-T5Pol (SEQ ID | G025991 | 0.0 | 0.0 | 2 |
| NO: 1135) | G029564 | 0.0 | 0.0 | 2 |
| | G029565 | 0.0 | 0.0 | 2 |
| | G029566 | 0.0* | N/A | 1 |
| | G029567 | 0.0 | 0.0 | 2 |
| | G029568 | 0.0 | 0.0 | 2 |
| | G029569 | 0.0 | 0.0 | 2 |
| | G029570 | 0.0 | 0.0 | 2 |
| | G029571 | 0.0 | 0.0 | 2 |
| | G029572 | 0.0 | 0.0 | 2 |
| polK-nCas9 (SEQ ID NO: | G025991 | 0.2 | 0.0 | 2 |
| 1025) | G029564 | 0.8* | N/A | 1 |
| | G029565 | 1.6 | 0.4 | 2 |
| | G029566 | 2.6 | 1.0 | 2 |
| | G029567 | 3.2 | 0.9 | 2 |
| | G029568 | 1.7 | 0.8 | 2 |
| | G029569 | 0.2 | 0.1 | 2 |
| | G029570 | 0.1 | 0.1 | 2 |
| | G029571 | 0.1 | 0.1 | 2 |
| | G029572 | 0.1 | 0.1 | 2 |
| nCas9-Phi29Pol (SEQ ID | G025991 | 0.1 | 0.1 | 2 |
| NO: 1193) | G029564 | 0.0* | N/A | 1 |
| | G029565 | 0.2 | 0.1 | 2 |
| | G029566 | 0.4 | 0.1 | 2 |
| | G029567 | 1.3 | 0.4 | 2 |
| | G029568 | 0.4 | 0.3 | 2 |
| | G029569 | 0.1 | 0.1 | 2 |
| | G029570 | 0.1 | 0.1 | 2 |
| | G029571 | 0.0 | 0.0 | 2 |
| | G029572 | 0.0 | 0.0 | 2 |
| nCas9-Phi29Pol(8M) ** | G025991 | 0.2 | 0.1 | 2 |
| (SEQ ID NO: 1196) | G029564 | 0.0 | 0.0 | 2 |
| | G029565 | 0.1 | 0.1 | 2 |
| | G029566 | 0.0 | 0.0 | 2 |
| | G029567 | 0.1 | 0.0 | 2 |
| | G029568 | 0.1 | 0.1 | 2 |
| | G029569 | 0.0 | 0.0 | 2 |
| | G029570 | 0.0 | 0.0 | 2 |
| | G029571 | 0.0 | 0.0 | 2 |
| | G029572 | 0.0 | 0.0 | 2 |

*Indicates experimental group for which N = 1
** Bars not visible in corresponding graph due to low editing.

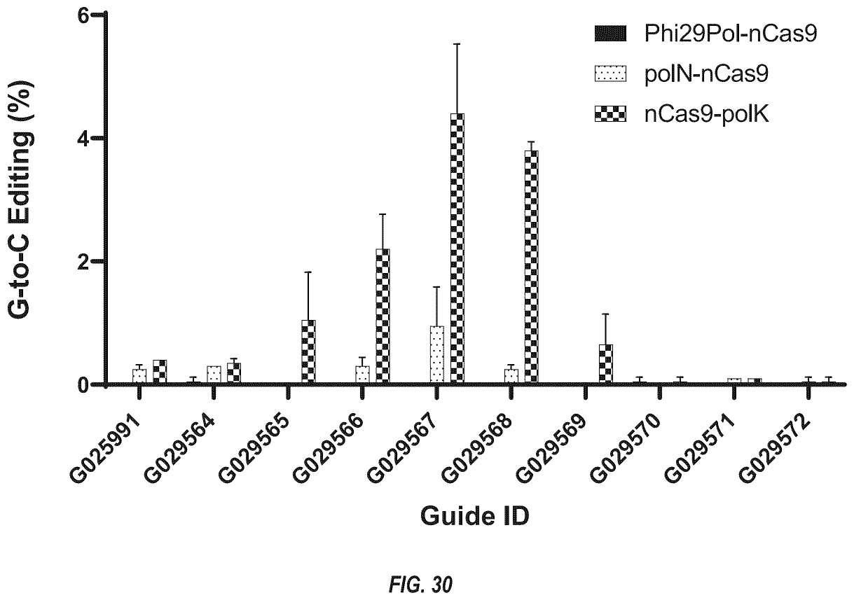

In a third experiment, another set of mRNAs encoding DNA polymerase-Cas9 fusion proteins were transfected in Huh7 cells as described above in combination with guides listed in Table 33 using the same protocol as described above. Samples were run in duplicate. Templated editing results are shown in Table 36 and FIG. 30.

TABLE 36
Templated editing at the HEK3 locus in Huh7 cells

| mRNA ID | Guide ID | G-to-C Editing (%) | SD |
|---|---|---|---|
| Phi29Pol-nCas9 (SEQ | G025991 | 0.0 | 0.0 |
| ID NO: 1190) | G029564 | 0.1 | 0.1 |
| | G029565 | 0.0 | 0.0 |
| | G029566 | 0.0 | 0.0 |
| | G029567 | 0.0 | 0.0 |
| | G029568 | 0.0 | 0.0 |
| | G029569 | 0.0 | 0.0 |
| | G029570 | 0.1 | 0.1 |
| | G029571 | 0.0 | 0.0 |
| | G029572 | 0.0 | 0.0 |
| polN-nCas9 (SEQ ID | G025991 | 0.3 | 0.1 |
| NO: 1092) | G029564 | 0.3 | 0.0 |
| | G029565 | 0.0 | 0.0 |
| | G029566 | 0.3 | 0.1 |
| | G029567 | 1.0 | 0.6 |
| | G029568 | 0.3 | 0.1 |
| | G029569 | 0.0 | 0.0 |
| | G029570 | 0.0 | 0.0 |
| | G029571 | 0.1 | 0.0 |
| | G029572 | 0.1 | 0.1 |
| nCas9-polK (SEQ ID | G025991 | 0.4 | 0.0 |
| NO: 1022) | G029564 | 0.4 | 0.1 |
| | G029565 | 1.1 | 0.8 |
| | G029566 | 2.2 | 0.6 |
| | G029567 | 4.4 | 1.1 |
| | G029568 | 3.8 | 0.1 |
| | G029569 | 0.7 | 0.5 |
| | G029570 | 0.1 | 0.1 |
| | G029571 | 0.1 | 0.0 |
| | G029572 | 0.1 | 0.1 |

### Example 12—Template Based Editing in Huh7 Cells, Various DNA Polymerases (DNA Polymerase K And Phi29 DNA Polymerase; Including Variants)

This Example evaluated templated editing efficiency with polk (e.g., full-length polK or polK(19-526)) or phi29 DNA polymerases. Additionally assessed were single polypeptide fusions with different C- to N-terminus arrangements. Each Cas9/DNA polymerase fusion was complexed with a tgRNA and assessed for in vitro template-based editing.

In vitro transcription was used to produce mRNAs encoding each of the fusion proteins as described in Example 1. Template guide RNAs included 3′ chimeric extensions differing in terms of their RNA and DNA contents as summarized in Table 33 of Example 11.

Template guide RNAs (tgRNAs) targeting the same target sequence in the HEK3 genomic locus with various 3′ chimeric DNA/RNA extensions encoding a G>C point mutation at the target locus were used. Guide extensions differed in their DNA and RNA contents. Guides were screened along with a panel of mRNAs encoding fusion proteins comprising a H840A SpyCas9 nickase (nCas9) moiety and a DNA polymerase moiety in a C-terminal to N-terminal or a N-terminal to C-terminal orientation. The mRNAs and guide RNAs were transfected into human hepatoma (Huh7) cells to determine editing efficiency as follows.

Cells were prepared as described in Example 1, plated at a density of 15,000 cells/well, and incubated for 24 hours at 37° C., 5% CO₂.

Lipofection of mRNAs and guide RNAs used pre-mixed lipid formulations as described in Example 1. The lipofection reagent contained a molar ratio of 50% Lipid A, 38% cholesterol, 9% DSPC, and 3% PEG2k-DMG, and the resulting N:P ratio of the lipid nucleic acid mixtures ranged from 6.5-7. In total, each transfection well contained 100 nM guide, 200 ng mRNA and 0.7 μL of pre-mixed lipid solution.

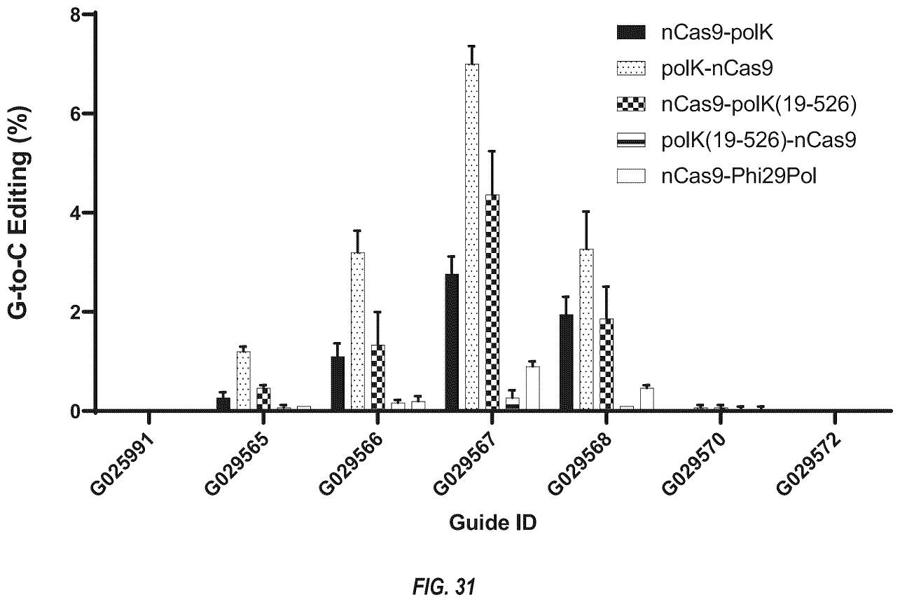

Cells were lysed 72 hours post-treatment, and genomic DNA was harvested and used for NGS sample preparation and bioinformatics analysis as described in Example 1. Samples were run in triplicate. Table 37 and FIG. 31 show the templated editing efficiencies of each combination of mRNA and tgRNA.

TABLE 37
Templated editing at the HEK3 locus in Huh7 cells using different
combinations of tgRNAs and mRNAs encoding fusion enzymes.

| mRNA ID | Guide ID | G-to-C Editing (%) | SD |
|---|---|---|---|
| nCas9-polK (SEQ ID NO: | G025991 | 0.0 | 0.0 |
| 1022) | G029565 | 0.3 | 0.1 |
| | G029566 | 1.1 | 0.3 |
| | G029567 | 2.8 | 0.4 |
| | G029568 | 2.0* | 0.4 |
| | G029570 | 0.0 | 0.0 |
| | G029572 | 0.0 | 0.0 |
| polK-nCas9 (SEQ ID NO: | G025991 | 0.0 | 0.0 |
| 1025) | G029565 | 1.2 | 0.1 |
| | G029566 | 3.2 | 0.4 |
| | G029567 | 7.0 | 0.4 |
| | G029568 | 3.3 | 0.8 |
| | G029570 | 0.1 | 0.1 |
| | G029572 | 0.0 | 0.0 |
| nCas9-polK(19-526) (SEQ ID | G025991 | 0.0 | 0.0 |
| NO: 1037) | G029565 | 0.5 | 0.1 |
| | G029566 | 1.3 | 0.7 |
| | G029567 | 4.4 | 0.9 |
| | G029568 | 1.9 | 0.6 |
| | G029570 | 0.1 | 0.1 |
| | G029572 | 0.0 | 0.0 |
| polK(19-526)-nCas9 (SEQ ID | G025991 | 0.0 | 0.0 |
| NO: 1040) | G029565 | 0.1 | 0.1 |
| | G029566 | 0.2 | 0.1 |
| | G029567 | 0.3 | 0.2 |
| | G029568 | 0.1 | 0.0 |
| | G029570 | 0.0 | 0.1 |
| | G029572 | 0.0 | 0.0 |
| nCas9-Phi29Pol (SEQ ID NO: | G025991 | 0.0 | 0.0 |
| 1193) | G029565 | 0.1 | 0.0 |
| | G029566 | 0.2 | 0.1 |
| | G029567 | 0.9 | 0.1 |
| | G029568 | 0.5 | 0.1 |
| | G029570 | 0.0 | 0.1 |
| | G029572 | 0.0 | 0.0 |

*Indicates experimental group for which N = 2

### Example 13—Template Based Editing in Huh7 and HEK293T Cells with DNA Polymerase K, Including Variants

This Example evaluated templated editing efficiency with a DNA polymerase-Cas9 fusion protein comprising a H840A nicking SpyCas9 (nCas9) moiety fused to a DNA-dependent DNA polymerase K moiety, or variants thereof (PolK variants with the amino acid substitution(s) R507A, R507G, or GE411-412RV).

Each of the DNA polymerase-Cas9 fusion proteins were screened in Huh7 and HEK293T cells for editing efficiency in conjunction with a tgRNA. In particular, this study assessed the following single polypeptide fusions including a DNA Polymerase K variant; nCas9-polK (R507A) (SEQ ID NO: 1031); nCas9-polK (R507G) (SEQ ID NO: 1034), and nCas9-polK(GE411-412RV) (SEQ ID NO: 1028). Additionally assessed were fusions including PolK and Cas9 in alternative arrangements (e.g., nCas9-polK (SEQ ID NO: 1022) and polK-nCas9 (SEQ ID NO: 1025)). Cas9/DNA polymerase fusion was complexed with one of six tgRNAs and assessed for in vitro template-based editing. Template guide RNAs included template regions and DNA polymerase recruiting sequence (DRS) regions of varying lengths (see, e.g., description of gRNAs in Table 29 of Example 10, and Table 38A).

Huh7 and HEK293T cell lines (ATCC) were cultured according to standard tissue culture procedures in media containing Dulbecco's Modified Eagle Medium (DMEM, Corning, Cat. 10-013-CV) containing 10% fetal bovine serum (Gibco, Cat. A13450) and penicillin-streptomycin antibiotic supplement (Gibco, Cat. 30-002-CI) at 1%. Huh7 cells were seeded in 96-well cell culture-treated plates (Corning, Cat. 353072) at a density of 15,000 cells/well. HEK293T cells were seeded in 96-well cell culture-treated plates (Corning, Cat. 353072) coated with poly-D-lysine solution (Gibco, A3890401) according to manufacturer instructions at a density of 32,000 cells/well. After 24 hours of incubation at 37° C., 5% CO₂, cells were transfected with a tgRNA and a mRNA encoding a fusion protein comprising a nCas9 moiety and a DNA polymerase moiety. The tgRNA comprised: 1) a spacer sequence targeting the HEK293 Site 3 locus, 2) a synthetic SpyCas9 guide scaffold, and 3) a 3′ RNA/DNA hybrid extension comprising a template sequence encoding a G>C point mutation at the target locus and a DRS. Different tgRNAs were screened, which had different template sequence and DRS lengths.

mRNAs and tgRNAs were transfected in Huh7 and HEK293T cells using Lipofectamine™ MessengerMAX™ Reagent (ThermoFisher, Cat. LMRNA001) according to manufacturer's instructions. Each transfection well contained 80 μL media, 20 μL Opti-MEM™ (Gibco, Cat. 31985088), 0.3 μL MessengerMAX™ reagent, 100 ng mRNA, and 50 nM tgRNA. In a separate set of experiments, a nicking guide RNA (ngRNA) was also transfected to the cells along with tgRNA and mRNA in a 3:2 tgRNA:ngRNA molar ratio. In these transfections, each well contained 80 μL media, 20 μL Opti-MEM™, 0.3 μL MessengerMAX™ reagent, 100 ng mRNA, 30 nM tgRNA, and 20 nM ngRNA (G025992). The cells were lysed 72-hours post-treatment for NGS analysis as described in Example 1. Samples were run in duplicates.

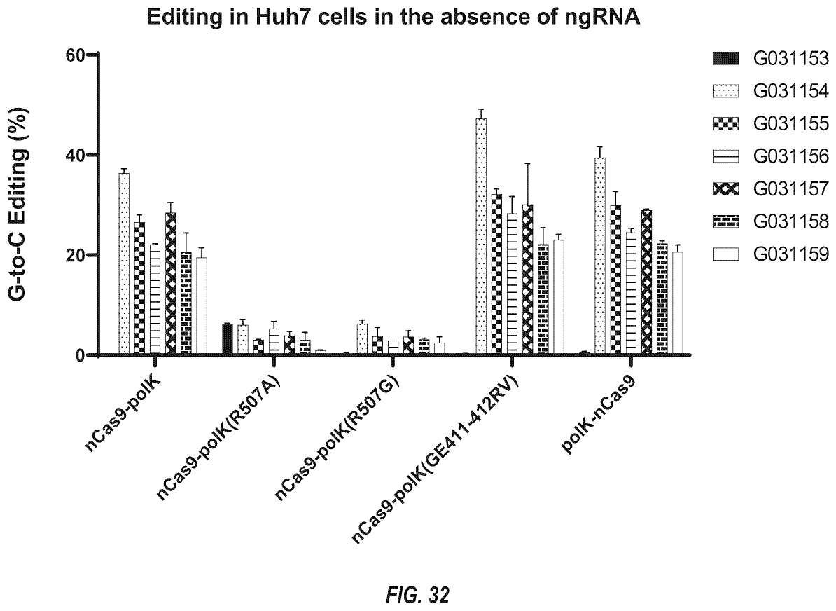

Table 38B and FIG. 32 show the templated editing efficiencies of each combination of genome editor mRNA and tgRNA in Huh7 cells.

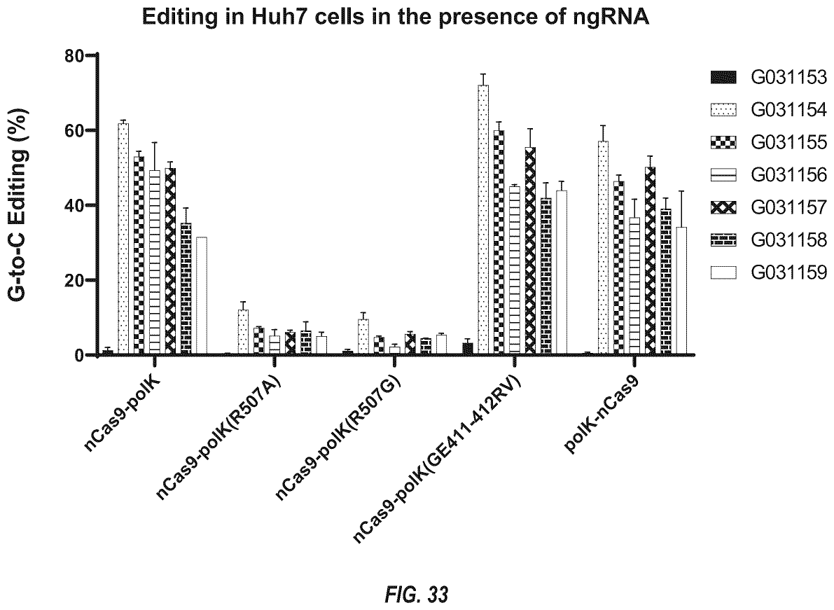

Table 39 and FIG. 33 show the templated editing efficiencies of each combination of genome editor mRNA and tgRNA in the presence of ngRNA in Huh7 cells.

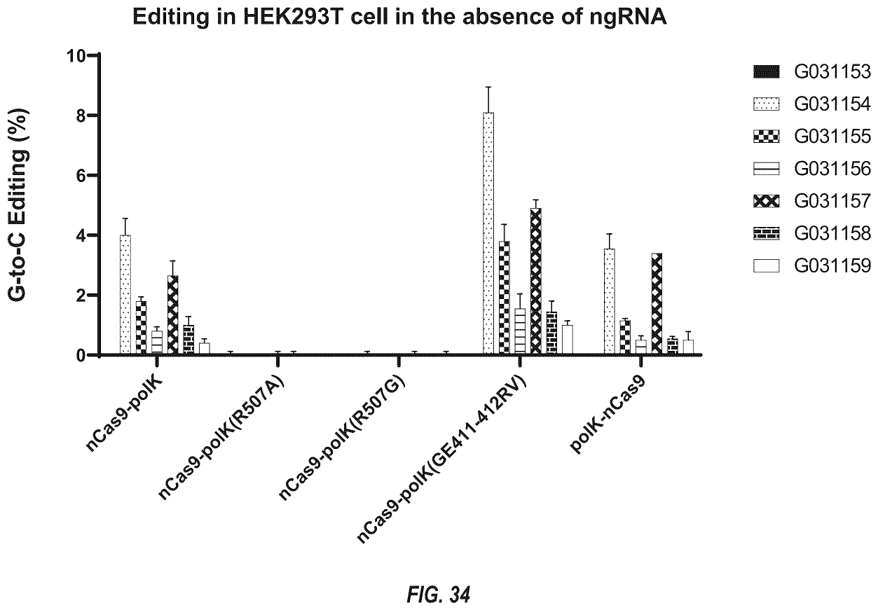

Table 40 and FIG. 34 show the templated editing efficiencies of each combination of genome editor mRNA and tgRNA in HEK293T cells.

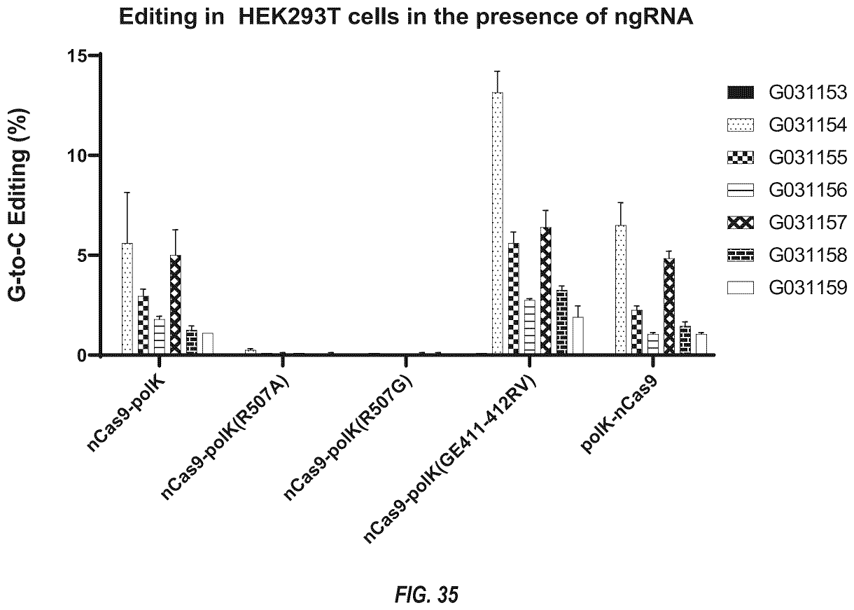

Table 41 and FIG. 35 show the templated editing efficiencies of each combination of genome editor mRNA and tgRNA in the presence of ngRNA in HEK293T cells.

TABLE 38A
Description of guide RNA Constructs

| Guide ID | 3′ Extension Sequence (DRS in bold) |
|---|---|
| G031153 (SEQ | UCCUCUGCGAUCACGUGCmUmCmA*mG*mU |
| ID NO: 479) | |
| G031154 (SEQ | dTdTdTdCdCdTdCdTdGdCdGdAdTdCdAdCGUGCmUmCmA*mG*mU |
| ID NO: 480) | |
| G031155 (SEQ | dTdCdCdTdCdTdGdCdGdAdTdCdAdCGUGCmUmCmA*mG*mU |
| ID NO: 503) | |
| G031156 (SEQ | dCdTdCdTdGdCdGdAdTdCdAdCGUGCmUmCmA*mG*mU |
| ID NO: 504) | |
| G031157 (SEQ | dTdTdTdCdCdTdCdTdGdCdGdAdTdCdAdCGUGCmUmCmAmGmU*mC*m |
| ID NO: 505) | U |
| G031158 (SEQ | dTdCdCdTdCdTdGdCdGdAdTdCdAdCGUGCmUmCmAmGmU*mC*mU |
| ID NO: 506) | |
| G031159 (SEQ | dCdTdCdTdGdCdGdAdTdCdAdCGUGCmUmCmAmGmU*mC*mU |
| ID NO: 507) | |

Note:
*denotes that the nucleotide is linked to the next nucleotide with a phosphorothioate (PS bond), a lower case “m” denotes that the nucleotide is modified with 2′-O-Me, and a lowercase “d” denotes that the nucleotide is a DNA nucleotide.

TABLE 38B
Templated editing efficiency at the HEK3 locus in Huh7 cells

| mRNA ID | Guide ID | G-to-C Editing (%) | SD |
|---|---|---|---|
| nCas9-polK (SEQ ID NO: | G031153 | 0.0 | 0.0 |
| 1022) | G031154 | 36.4 | 0.9 |
| | G031155 | 26.6 | 1.5 |
| | G031156 | 22.1 | 0.2 |
| | G031157 | 28.5 | 2.1 |
| | G031158 | 20.6 | 3.9 |
| | G031159 | 19.5 | 2.0 |
| nCas9-polK (R507A) (SEQ | G031153 | 6.1 | 0.3 |
| ID NO: 1031) | G031154 | 6.0 | 1.1 |
| | G031155 | 3.0 | 0.1 |
| | G031156 | 5.3 | 1.5 |
| | G031157 | 3.9 | 0.8 |
| | G031158 | 3.0 | 1.6 |
| | G031159 | 0.9 | 0.1 |
| nCas9-polK (R507G) (SEQ | G031153 | 0.2 | 0.3 |
| ID NO: 1034) | G031154 | 6.3 | 0.8 |
| | G031155 | 3.7 | 1.8 |
| | G031156 | 2.9 | 0.0 |
| | G031157 | 3.7 | 1.2 |
| | G031158 | 3.2 | 0.2 |
| | G031159 | 2.5 | 1.2 |
| nCas9-polK(GE411-412RV) | G031153 | 0.3 | 0.1 |
| (SEQ ID NO: 1028) | G031154 | 47.3 | 1.9 |
| | G031155 | 32.2 | 1.1 |
| | G031156 | 28.3 | 3.4 |
| | G031157 | 30.1 | 8.2 |
| | G031158 | 22.2 | 3.3 |
| | G031159 | 23.0 | 1.1 |
| polK-nCas9 (SEQ ID NO: | G031153 | 0.7 | 0.1 |
| 1025) | G031154 | 39.5 | 2.2 |
| | G031155 | 30.0 | 2.8 |
| | G031156 | 24.5 | 0.8 |
| | G031157 | 29.0 | 0.2 |
| | G031158 | 22.3 | 0.6 |
| | G031159 | 20.6 | 1.4 |

TABLE 39
Templated editing efficiency at the HEK3 locus
in Huh7 cells in the presence of ngRNA

| | | | G-to-C Editing | |
|---|---|---|---|---|
| mRNA ID | Guide ID | ngRNA | (%) | SD |
| nCas9-polK (SEQ ID | G031153 | G025992 | 1.3 | 0.8 |
| NO: 1022) | G031154 | G025992 | 61.9 | 0.9 |
| | G031155 | G025992 | 53.0 | 1.4 |
| | G031156 | G025992 | 49.4 | 7.4 |
| | G031157 | G025992 | 50.0 | 1.6 |
| | G031158 | G025992 | 35.3 | 4.0 |
| | G031159 | G025992 | 31.5* | N/A |
| nCas9-polK (R507A) | G031153 | G025992 | 0.5 | 0.1 |
| (SEQ ID NO: 1031) | G031154 | G025992 | 12.1 | 2.1 |
| | G031155 | G025992 | 7.3 | 0.4 |
| | G031156 | G025992 | 5.2 | 1.6 |
| | G031157 | G025992 | 6.1 | 0.6 |
| | G031158 | G025992 | 6.6 | 2.3 |
| | G031159 | G025992 | 5.1 | 1.1 |
| nCas9-polK (R507G) | G031153 | G025992 | 1.1 | 0.4 |
| (SEQ ID NO: 1034) | G031154 | G025992 | 9.6 | 1.8 |
| | G031155 | G025992 | 4.8 | 0.3 |
| | G031156 | G025992 | 2.2 | 0.7 |
| | G031157 | G025992 | 5.7 | 0.6 |
| | G031158 | G025992 | 4.5 | 0.1 |
| | G031159 | G025992 | 5.4 | 0.4 |
| nCas9-polK(GE411- | G031153 | G025992 | 3.2 | 1.1 |
| 412RV) (SEQ ID NO: | G031154 | G025992 | 72.1 | 3.0 |
| 1028) | G031155 | G025992 | 60.0 | 2.3 |
| | G031156 | G025992 | 45.1 | 0.5 |
| | G031157 | G025992 | 55.5 | 4.9 |
| | G031158 | G025992 | 42.0 | 4.0 |
| | G031159 | G025992 | 44.0 | 2.5 |
| polK-nCas9 (SEQ ID | G031153 | G025992 | 0.6 | 0.2 |
| NO: 1025) | G031154 | G025992 | 57.2 | 4.2 |
| | G031155 | G025992 | 46.5 | 1.6 |
| | G031156 | G025992 | 36.7 | 4.9 |
| | G031157 | G025992 | 50.2 | 3.0 |
| | G031158 | G025992 | 39.1 | 2.9 |
| | G031159 | G025992 | 34.2 | 9.7 |

*Indicates experimental group for which N = 1

TABLE 40
Templated editing efficiency at the HEK3 locus in HEK293T cells

| mRNA ID | Guide ID | G-to-C Editing (%) | SD |
|---|---|---|---|
| nCas9-polK (SEQ ID NO: | G031153 | 0.0 | 0.0 |
| 1022) | G031154 | 4.0 | 0.6 |
| | G031155 | 1.8 | 0.1 |
| | G031156 | 0.8 | 0.1 |
| | G031157 | 2.7 | 0.5 |
| | G031158 | 1.0 | 0.3 |
| | G031159 | 0.4 | 0.1 |
| nCas9-polK (R507A) (SEQ | G031153 | 0.1 | 0.1 |
| ID NO: 1031) | G031154 | 0.0 | 0.0 |
| | G031155 | 0.0 | 0.0 |
| | G031156 | 0.1 | 0.1 |
| | G031157 | 0.1 | 0.1 |
| | G031158 | 0.0 | 0.0 |
| | G031159 | 0.0 | 0.0 |
| nCas9-polK (R507G) (SEQ | G031153 | 0.0 | 0.0 |
| ID NO: 1034) | G031154 | 0.1 | 0.1 |
| | G031155 | 0.0 | 0.0 |
| | G031156 | 0.0 | 0.0 |
| | G031157 | 0.1 | 0.1 |
| | G031158 | 0.0 | 0.0 |
| | G031159 | 0.1 | 0.1 |
| nCas9-polK(GE411-412RV) | G031153 | 0.0 | 0.0 |
| (SEQ ID NO: 1028) | G031154 | 8.1 | 0.8 |
| | G031155 | 3.8 | 0.6 |
| | G031156 | 1.6 | 0.5 |
| | G031157 | 4.9 | 0.3 |
| | G031158 | 1.5 | 0.4 |
| | G031159 | 1.0 | 0.1 |
| polK-nCas9 (SEQ ID NO: | G031153 | 0.0 | 0.0 |
| 1025) | G031154 | 3.6 | 0.5 |
| | G031155 | 1.2 | 0.1 |
| | G031156 | 0.5 | 0.1 |
| | G031157 | 3.4 | 0.0 |
| | G031158 | 0.6 | 0.1 |
| | G031159 | 0.5 | 0.3 |

TABLE 41
Templated editing efficiency at the HEK3 locus
in HEK293T cells in the presence of ngRNA

| mRNA ID | Guide ID | ngRNA | G-to-C Editing (%) | SD |
|---|---|---|---|---|
| nCas9-polK (SEQ ID | G031153 | G025992 | 0.0 | 0.0 |
| NO: 1022) | G031154 | G025992 | 5.6 | 2.5 |
| | G031155 | G025992 | 3.0 | 0.4 |
| | G031156 | G025992 | 1.8 | 0.1 |
| | G031157 | G025992 | 5.0 | 1.3 |
| | G031158 | G025992 | 1.3 | 0.2 |
| | G031159 | G025992 | 1.1 | 0.0 |
| nCas9-polK (R507A) | G031153 | G025992 | 0.0 | 0.0 |
| (SEQ ID NO: 1031) | G031154 | G025992 | 0.3 | 0.1 |
| | G031155 | G025992 | 0.1 | 0.0 |
| | G031156 | G025992 | 0.1 | 0.1 |
| | G031157 | G025992 | 0.1 | 0.0 |
| | G031158 | G025992 | 0.0 | 0.0 |
| | G031159 | G025992 | 0.1 | 0.1 |
| nCas9-polK (R507G) | G031153 | G025992 | 0.0 | 0.0 |
| (SEQ ID NO: 1034) | G031154 | G025992 | 0.1 | 0.0 |
| | G031155 | G025992 | 0.0 | 0.0 |
| | G031156 | G025992 | 0.0 | 0.0 |
| | G031157 | G025992 | 0.1 | 0.1 |
| | G031158 | G025992 | 0.1 | 0.1 |
| | G031159 | G025992 | 0.0 | 0.0 |
| nCas9-polK(GE411- | G031153 | G025992 | 0.1 | 0.0 |
| 412RV) (SEQ ID NO: | G031154 | G025992 | 13.2 | 1.1 |
| 1028) | G031155 | G025992 | 5.6 | 0.6 |
| | G031156 | G025992 | 2.8 | 0.1 |
| | G031157 | G025992 | 6.4 | 0.8 |
| | G031158 | G025992 | 3.3 | 0.2 |
| | G031159 | G025992 | 1.9 | 0.6 |
| polK-nCas9 (SEQ ID | G031153 | G025992 | 0.0 | 0.0 |
| NO: 1025) | G031154 | G025992 | 6.5 | 1.1 |
| | G031155 | G025992 | 2.3 | 0.2 |
| | G031156 | G025992 | 1.1 | 0.1 |
| | G031157 | G025992 | 4.9 | 0.4 |
| | G031158 | G025992 | 1.5 | 0.2 |
| | G031159 | G025992 | 1.1 | 0.1 |

### Example 14—Template Based Editing in Huh7 Cells with DNA Polymerase K, Including Variants

In this Example, DNA polymerase-Cas9 fusion protein comprised of a H840A nicking SpyCas9 (nCas9) moiety fused to variants of DNA polymerase kappa (e.g., polK variants with amino acid substitution(s) at R507G or GE411-412RV) were screened in Huh7 cells. Further, the impact of different levels of total cargo of RNA (mRNA and guide RNA) on in vitro genome editing efficiency was assessed.

Further, the following DNA polymerase-Cas9 fusion proteins with DNA Polymerase K variants were assessed: nCas9-polK (R507G) (SEQ ID NO: 1034) and nCas9-polK(GE411-412RV) (SEQ ID NO: 1028). Additionally assessed were fusions including PolK and Cas9 in alternative arrangements (e.g., nCas9-polK (SEQ ID NO: 1022) and polK-nCas9 (SEQ ID NO: 1025)). Each Cas9/DNA polymerase fusion was complexed with a tgRNA and assessed for in vitro template-based editing.

DNA polymerase-Cas9 fusion protein comprised of a H840A nicking SpyCas9 (nCas9) moiety fused to variants of DNA polymerase kappa were screened in Huh7 cells. Cells were prepared as described in Example 1, plated at a density of 15,000 cells/well, and incubated for 24 hours at 37° C., 5% CO₂. Cells were transfected with mRNAs encoding a genome editor along with guide RNAs using Lipofectamine™ MessengerMAX™ Reagent (Thermofisher, Cat. LMRNA001) according to manufacturer's protocol in a 2:1 mRNA to guide RNA ratio by weight. The guide RNAs transfected comprised a tgRNA (G031154) comprising a hybrid RNA/DNA 3′ extension encoding a G>C transversion mutation at the HEK293 site 3 genomic locus and a ngRNA (G025992) in a tgRNA:ngRNA ratio of 3:2 by weight. The total cargo of RNA (mRNA and guide RNA) delivered was diluted in growth media in a two-fold, 7-point dilution series where the highest dose condition was 400 ng of total RNA, 0.3 μL of MessengerMAX™ reagent, and 20 μL Opti-MEM™ (Gibco, Cat. 31985088) in growth media to a total volume of 100 μL per transfection well. Samples were run in duplicates. The cells were lysed 72 hours post-treatment for NGS analysis as described in Example 1.

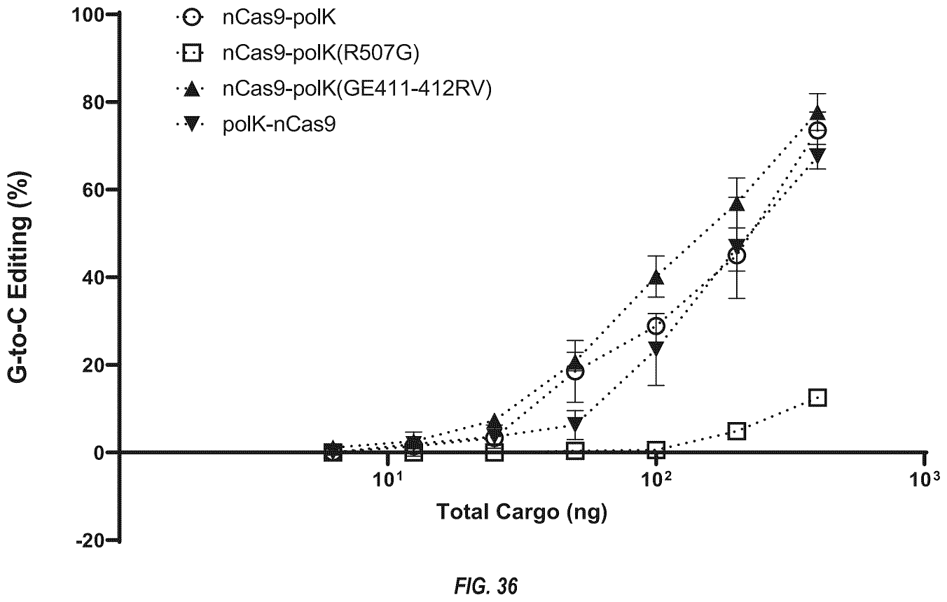

Table 42 and FIG. 36 show the templated editing efficiency and standard deviation as obtained from the dose response analysis.

TABLE 42
Templated editing at the HEK3 locus in Huh7 cells.

| | Guide | ngRNA | Total | G-to-C | |
|---|---|---|---|---|---|
| mRNA ID | RNA ID | ID | Cargo (ng) | Editing (%) | SD |
| nCas9-polK | G031154 | G025992 | 400 | 73.5 | 4.2 |
| (SEQ ID NO: | G031154 | G025992 | 200 | 45.1 | 3.6 |
| 1022) | G031154 | G025992 | 100 | 28.9 | 1.0 |
| | G031154 | G025992 | 50 | 18.5 | 7.1 |
| | G031154 | G025992 | 25 | 3.3 | 0.7 |
| | G031154 | G025992 | 12.5 | 1.5 | 0.4 |
| | G031154 | G025992 | 6.25 | 0.0 | 0.0 |
| | G031154 | G025992 | 0 | 0.0 | 0.0 |
| nCas9- | G031154 | G025992 | 400 | 12.5 | 1.1 |
| polK(R507G) | G031154 | G025992 | 200 | 4.9 | 1.1 |
| (SEQ ID NO: | G031154 | G025992 | 100 | 0.6 | 0.8 |
| 1034) | G031154 | G025992 | 50 | 0.4 | 0.4 |
| | G031154 | G025992 | 25 | 0.0 | 0.0 |
| | G031154 | G025992 | 12.5 | 0.0 | 0.0 |
| | G031154 | G025992 | 6.25 | 0.0 | 0.0 |
| | G031154 | G025992 | 0 | 0.0 | 0.0 |
| nCas9- | G031154 | G025992 | 400 | 77.7 | 4.2 |
| polK(GE411- | G031154 | G025992 | 200 | 57.0 | 5.7 |
| 412RV) (SEQ | G031154 | G025992 | 100 | 40.2 | 4.7 |
| ID NO: 1028) | G031154 | G025992 | 50 | 20.8 | 2.1 |
| | G031154 | G025992 | 25 | 7.3 | 0.7 |
| | G031154 | G025992 | 12.5 | 2.6 | 0.4 |
| | G031154 | G025992 | 6.25 | 1.2 | 1.2 |
| | G031154 | G025992 | 0 | 0.0 | 0.0 |
| polK-nCas9 | G031154 | G025992 | 400 | 67.5 | 2.8 |
| (SEQ ID NO: | G031154 | G025992 | 200 | 46.7 | 11.6 |
| 1025) | G031154 | G025992 | 100 | 23.5 | 8.2 |
| | G031154 | G025992 | 50 | 6.3 | 3.3 |
| | G031154 | G025992 | 25 | 3.6 | 2.7 |
| | G031154 | G025992 | 12.5 | 2.0 | 2.8 |
| | G031154 | G025992 | 6.25 | 0.0 | 0.0 |
| | G031154 | G025992 | 0 | 0.0 | 0.0 |

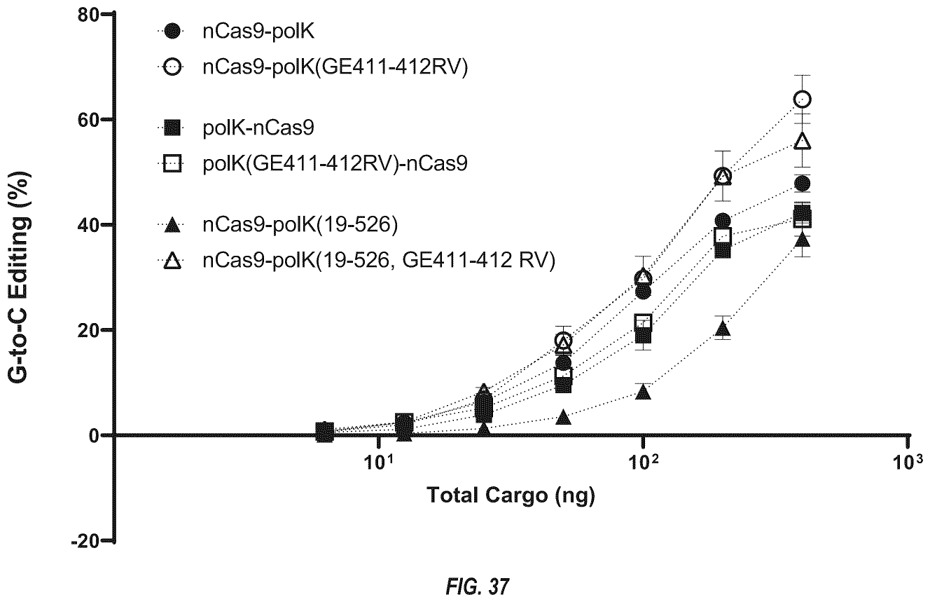

In a separate experiment, genome editors comprised of nCas9 fused to variants of DNA polymerase kappa were used for editing in Huh7 cells in combination with tgRNA G031154 and ngRNA G025992 according to the same protocol as described above. Samples were run in triplicate. Table 43 and FIG. 37 show the templated editing efficiency of these mRNA constructs at the HEK3 locus in Huh7 cells.

TABLE 43
Templated editing at the HEK3 locus in Huh7 cells.

| | | ngRNA | Total | G-to-C | |
|---|---|---|---|---|---|
| mRNA ID | Guide ID | ID | Cargo (ng) | Editing (%) | SD |
| nCas9-polK | G031154 | G025992 | 400 | 47.9 | 1.6 |
| (SEQ ID NO: | G031154 | G025992 | 200 | 40.8 | 1.2 |
| 1022) | G031154 | G025992 | 100 | 27.3 | 1.3 |
| | G031154 | G025992 | 50 | 13.8 | 1.2 |
| | G031154 | G025992 | 25 | 6.4 | 1.6 |
| | G031154 | G025992 | 12.5 | 2.4 | 1.1 |
| | G031154 | G025992 | 6.25 | 0.8 | 0.7 |
| | G031154 | G025992 | 0 | 0.0 | 0.1 |
| nCas9- | G031154 | G025992 | 400 | 63.8 | 4.6 |
| polK(GE411- | G031154 | G025992 | 200 | 49.3 | 4.8 |
| 412RV) (SEQ ID | G031154 | G025992 | 100 | 29.7 | 0.3 |
| NO: 1028) | G031154 | G025992 | 50 | 18.0 | 2.7 |
| | G031154 | G025992 | 25 | 6.8 | 2.3 |
| | G031154 | G025992 | 12.5 | 2.0 | 0.7 |
| | G031154 | G025992 | 6.25 | 0.7 | 0.2 |
| | G031154 | G025992 | 0 | 0.0 | 0.1 |
| polK-nCas9 | G031154 | G025992 | 400 | 42.3 | 1.9 |
| (SEQ ID NO: | G031154 | G025992 | 200 | 35.2 | 1.5 |
| 1025) | G031154 | G025992 | 100 | 19.0 | 2.8 |
| | G031154 | G025992 | 50 | 9.6 | 0.5 |
| | G031154 | G025992 | 25 | 3.9 | 1.4 |
| | G031154 | G025992 | 12.5 | 1.1 | 0.1 |
| | G031154 | G025992 | 6.25 | 0.6 | 0.3 |
| | G031154 | G025992 | 0 | 0.0 | 0.0 |
| polK(GE411- | G031154 | G025992 | 400 | 41.1 | 3.2 |
| 412RV)-nCas9 | G031154 | G025992 | 200 | 37.8 | 2.5 |
| (SEQ ID NO: | G031154 | G025992 | 100 | 21.4 | 0.2 |
| 1043) | G031154 | G025992 | 50 | 11.3 | 0.5 |
| | G031154 | G025992 | 25 | 5.2 | 0.6 |
| | G031154 | G025992 | 12.5 | 2.5 | 0.1 |
| | G031154 | G025992 | 6.25 | 0.7 | 0.3 |
| | G031154 | G025992 | 0 | 0.0 | 0.0 |
| nCas9-polK(19- | G031154 | G025992 | 400 | 37.4 | 3.5 |
| 526) (SEQ ID | G031154 | G025992 | 200 | 20.4 | 2.3 |
| NO: 1037) | G031154 | G025992 | 100 | 8.3 | 1.5 |
| | G031154 | G025992 | 50 | 3.6 | 1.2 |
| | G031154 | G025992 | 25 | 1.3 | 0.2 |
| | G031154 | G025992 | 12.5 | 0.4 | 0.3 |
| | G031154 | G025992 | 6.25 | 0.1 | 0.1 |
| | G031154 | G025992 | 0 | 0.0 | 0.0 |
| nCas9-polK(19- | G031154 | G025992 | 400 | 56.0 | 5.1 |
| 526, GE411- | G031154 | G025992 | 200 | 49.2 | 1.4 |
| 412RV) (SEQ ID | G031154 | G025992 | 100 | 30.3 | 3.7 |
| NO: 1046) | G031154 | G025992 | 50 | 17.2 | 2.0 |
| | G031154 | G025992 | 25 | 8.3 | 1.0 |
| | G031154 | G025992 | 12.5 | 2.5 | 0.1 |
| | G031154 | G025992 | 6.25 | 1.1 | 0.4 |
| | G031154 | G025992 | 0 | 0.0 | 0.0 |

### Example 15—Templated Editing in HEK293T Cells Using LNP Delivery with DNA Polymerase K and T5 DNA Polymerase; Including Variants

In this Example, template based editing ability of fusion proteins comprising a nCas9 moiety and a DNA polymerase moiety was tested in HEK293T cells. Further, the impact of different levels of total cargo of RNA (mRNA and guide RNA) on in vitro genome editing efficiency at an exemplary target locus (HEK3) was assessed.

Single polypeptide fusions were evaluated that included nCas9 operably linked to one of the following DNA polymerases: polK (e.g., polk-nCas9 (SEQ ID NO: 1025) or nCas9-polK (SEQ ID NO: 1022)) or T5 (e.g., nCas9-T5Pol(D164A,E166A; SEQ ID NO: 1130). Each fusion protein was assessed in combination with template guide RNAs (tgRNAs) comprising hybrid 3′ extensions which differed by DNA polymerase recruiting sequence (DRS) and template sequence lengths with or without a nicking guide RNA (ngRNA).

HEK293T cells (ATCC, lot #70049877) were thawed and resuspended in complete media containing 1× Dulbecco's Modification of Eagle's Medium with 4.5 g/L glucose, L-glutamine & sodium pyruvate (Corning, Cat. 10-013-CV), FBS (Gibco, Cat. A13450) at 10%, and penicillin-streptomycin antibiotic supplement (Gibco, Cat. 30-002-CI) at 1% and passaged following centrifugation and resuspension. Cells were trypsinized, then counted and plated on 96-well cell culture-treated plates (Corning, Cat. 353072) coated with poly-D-lysine solution (Gibco, A3890401) according to manufacturer instructions at a density of 32,000 cells/well. Cells were incubated in a tissue culture incubator at 37° C. and 5% CO₂ atmosphere for 24 hours.

Lipid nanoparticles (LNPs) comprising a single RNA species were prepared as described in Example 1, with a molar ratio of 50% Lipid A, 38% cholesterol, 9% DSPC, and 3% PEG2k-DMG. Briefly, cells were incubated at 37° C., 5% CO₂ for 24 hours prior to treatment with LNPs. LNPs were incubated in media containing 10% fetal bovine serum (FBS) at 37° C. for 10 minutes. Post-incubation, LNPs comprising mRNA encoding a fusion protein comprising a nCas9 moiety and a DNA polymerase moiety, LNPs comprising a tgRNA, and LNPs comprising a ngRNA were delivered to the cells in a guide RNA: mRNA ratio of 1:2 by weight. In experimental groups treated without ngRNA, the guide RNA amount consisted only of tgRNA. In experimental groups treated with ngRNA, the guide RNA amount comprised both tgRNA and ngRNA in a 3:2 tgRNA:ngRNA mass ratio. The total RNA cargo (mRNA+guide RNA) was serially diluted in a seven-point, two-fold dilution series starting at a highest dose of 400 ng. Samples were run in triplicate. Cells were lysed 72 hours post-treatment for NGS analysis as described in Example 1.

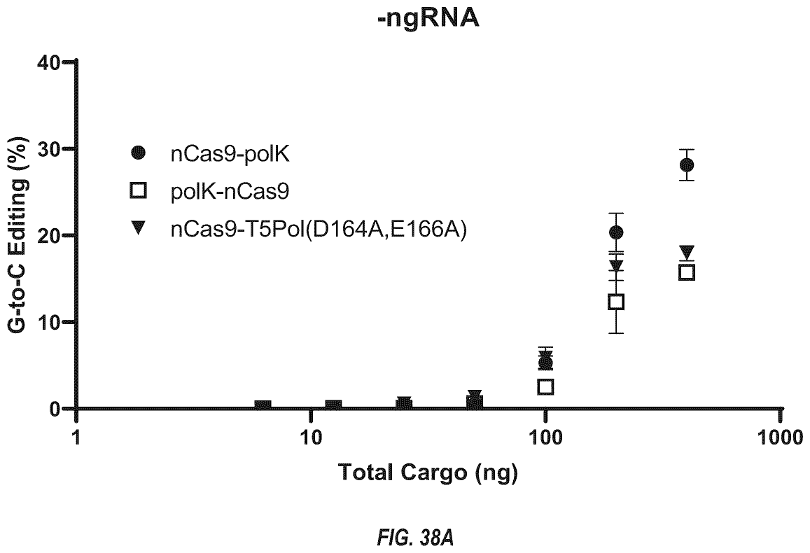

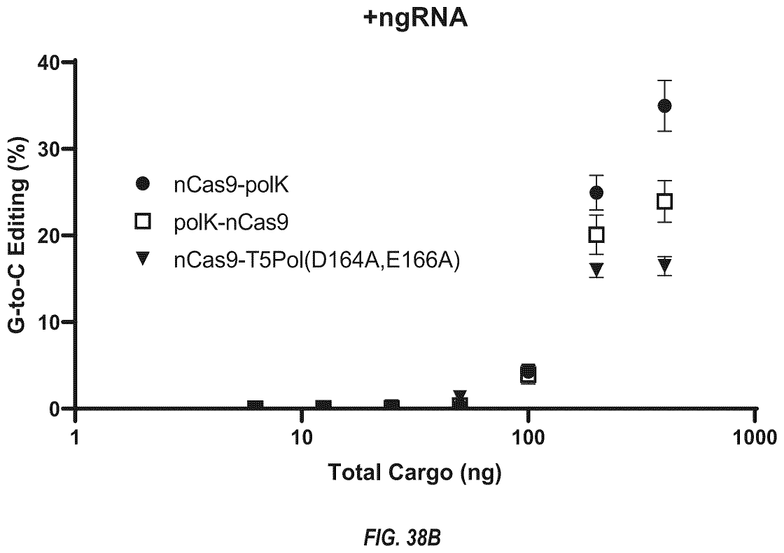

Table 44 and FIGS. 38A-38B show template editing efficiency of each combination of mRNA and tgRNA with or without ngRNA.

TABLE 44
Templated editing at the HEK3 locus in HEK293T cells

| | | Nicking | Total Cargo | G-to-C | |
|---|---|---|---|---|---|
| mRNA ID | Guide ID | Guide ID | (ng) | Editing (%) | SD |
| nCas9-polK | G031154 | — | 400 | 28.1 | 1.8 |
| (SEQ ID | G031154 | — | 200 | 20.4 | 2.2 |
| NO: 1022) | G031154 | — | 100 | 5.3 | 0.8 |
| | G031154 | — | 50 | 0.6 | 0.1 |
| | G031154 | — | 25 | 0.2 | 0.1 |
| | G031154 | — | 12.5 | 0.0 | 0.1 |
| | G031154 | — | 6.25 | 0.0 | 0.0 |
| polK-nCas9 | G031154 | — | 400 | 15.7 | 0.6 |
| (SEQ ID | G031154 | — | 200 | 12.3 | 3.6 |
| NO: 1025) | G031154 | — | 100 | 2.5 | 0.5 |
| | G031154 | — | 50 | 0.6 | 0.4 |
| | G031154 | — | 25 | 0.1 | 0.1 |
| | G031154 | — | 12.5 | 0.0 | 0.1 |
| | G031154 | — | 6.25 | 0.0 | 0.0 |
| nCas9- | G031157 | — | 400 | 17.9 | 0.9 |
| T5Pol(D164A, | G031157 | — | 200 | 16.3 | 1.5 |
| E166A) | G031157 | — | 100 | 5.9 | 1.2 |
| (SEQ ID | G031157 | — | 50 | 1.4 | 0.1 |
| NO: 1130) | G031157 | — | 25 | 0.6 | 0.4 |
| | G031157 | — | 12.5 | 0.2 | 0.2 |
| | G031157 | — | 6.25 | 0.1 | 0.1 |
| nCas9-polK | G031154 | G029552 | 400 | 35.0 | 2.9 |
| (SEQ ID | G031154 | G029552 | 200 | 24.9 | 2.0 |
| NO: 1022) | G031154 | G029552 | 100 | 4.3 | 0.7 |
| | G031154 | G029552 | 50 | 0.3 | 0.2 |
| | G031154 | G029552 | 25 | 0.1 | 0.1 |
| | G031154 | G029552 | 12.5 | 0.0 | 0.0 |
| | G031154 | G029552 | 6.25 | 0.0 | 0.0 |
| polK-nCas9 | G031154 | G029552 | 400 | 23.9 | 2.4 |
| (SEQ ID | G031154 | G029552 | 200 | 20.1 | 2.3 |
| NO: 1025) | G031154 | G029552 | 100 | 3.9 | 1.1 |
| | G031154 | G029552 | 50 | 0.4 | 0.2 |
| | G031154 | G029552 | 25 | 0.0 | 0.1 |
| | G031154 | G029552 | 12.5 | 0.0 | 0.1 |
| | G031154 | G029552 | 6.25 | 0.0 | 0.0 |
| nCas9- | G031157 | G029552 | 400 | 16.5 | 1.1 |
| T5Pol(D164A, | G031157 | G029552 | 200 | 16.0 | 0.8 |
| E166A) | G031157 | G029552 | 100 | 4.4 | 0.2 |
| (SEQ ID | G031157 | G029552 | 50 | 1.3 | 0.3 |
| NO: 1130) | G031157 | G029552 | 25 | 0.3 | 0.2 |
| | G031157 | G029552 | 12.5 | 0.1 | 0.1 |
| | G031157 | G029552 | 6.25 | 0.1 | 0.1 |

### Example 16—Template Based Editing in Human CD34+ Cells with DNA Polymerase K Including Variants

In this Example, templated editing with a DNA polymerase-Cas9 fusion (e.g., nCas9-POLK (GE411-412RV)) was evaluated in CD34+ cells. The DNA polymerase-Cas9 fusion was complexed with a template guide RNA (tgRNA, G032595; SEQ ID NO: 526) comprising (i) a spacer sequence targeting the VEGFA locus, and (ii) a hybrid RNA/DNA 3′ extension comprising a 12-nt DNA polymerase recruiting sequence (DRS) and a 18-nt template sequence. The template sequence encoded a 3-nucleotide substitution mutation (GAGG>CACT), with or without a nicking guide RNA (ngRNA).

CD34+ cells were thawed and resuspended in StemSpan™ Serum Free Expansion Media (SFEM II, STEMCELL Technologies, Cat. 09655) with added StemSpan CC100 cytokine cocktail (STEMCELL Technologies, Cat. 02690), penicillin-streptomycin antibiotic supplement (Gibco, Cat. 30-002-CI) at 1%, and 7.4 μM AhR Antagonist II SR-1 (Millipore Sigma, Cat. 182706). Cells were incubated for 3 days at 37° C., 5% CO₂ prior to use.

Cells were transfected with mRNA encoding a H840A SpyCas9 nickase moiety fused to a DNA polymerase moiety, and with guide RNA (either a tgRNA alone, or both a tgRNA and a ngRNA). Mixtures of cells, and mRNA and guide RNA combinations were prepared using the P3 Primary Cell 96-well Nucleofector Kit (Lonza, Cat. V4SP-3960) according to manufacturer's instructions. Each electroporation well contained 9.375-300 ng mRNA, 2 μM guide RNA, and 50,000 CD34+ cells in P3 buffer to a total volume of 20 μL. Each well was electroporated using the Amaxa 96-well Shuttle System (Lonza, CB00025797) with the CL-120 protocol and 80 μL of complete SFEM II media was added to each well. Cells were incubated at 37° C., 5% CO₂ for 15 min then transferred to corresponding wells of a 96-well U-bottom plate (Corning, Cat. 07-200-95) containing an equal amount of complete media. Following 24 hours of incubation, cells were centrifuged at 450 g for 5 minutes, media was removed and replaced. Following an additional 72 hours of incubation at 37° C., 5% CO2, cells were pelleted by centrifugation at 450 g for 5 min. Media was decanted and genomic DNA was harvested from cell pellets for NGS editing efficiency analysis as described in Example 1.

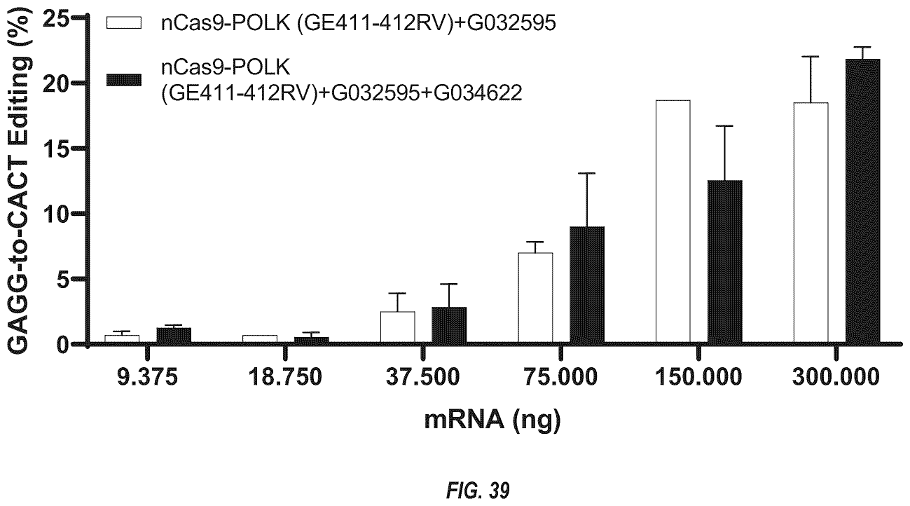

Table 45 and FIG. 39 show templated editing efficiency of different combinations of mRNA and guide RNA(s).

TABLE 45
Templated editing at the VEGFA locus in CD34+ cells

| | | | | | | GAGG- | | |
|---|---|---|---|---|---|---|---|---|
| | mRNA | Guide | Guide | ngRNA | ngRNA | to-CACT | | |
| mRNA ID | (ng) | ID | (μM) | ID | (μM) | Editing (%) | SD | N |
| nCas9-POLK | 300 | G032595 | 2 | — | — | 18.5% | 3.5% | 2 |
| (GE411-412RV) | 150 | G032595 | 2 | — | — | 18.7%* | N/A | 1 |
| (SEQ ID NO: | 75 | G032595 | 2 | — | — | 7.0% | 0.8% | 2 |
| 1028) | 37.5 | G032595 | 2 | — | — | 2.5% | 1.4% | 2 |
| | 18.75 | G032595 | 2 | — | — | 0.7%* | N/A | 1 |
| | 9.375 | G032595 | 2 | — | — | 0.7% | 0.3% | 2 |
| nCas9-POLK | 300 | G032595 | 1.2 | G034622 | 0.8 | 21.9% | 0.9% | 2 |
| (GE411-412RV) | 150 | G032595 | 1.2 | G034622 | 0.8 | 12.6% | 4.2% | 2 |
| (SEQ ID NO: | 75 | G032595 | 1.2 | G034622 | 0.8 | 9.0% | 4.1% | 2 |
| 1028) | 37.5 | G032595 | 1.2 | G034622 | 0.8 | 2.9% | 1.8% | 2 |
| | 18.75 | G032595 | 1.2 | G034622 | 0.8 | 0.6% | 0.4% | 2 |
| | 9.375 | G032595 | 1.2 | G034622 | 0.8 | 1.3% | 0.2% | 2 |

*indicates experimental group for which N = 1

### Example 17—Templated Editing in Huh7 Cells with Various DNA-Dependent DNA Polymerases (Polymerase θ, Klenow, Phi29 Polymerase, Polymerase N, and T5 DNA Polymerase; Including Variants)

Template based editing can be performed using two separate mRNAs, one encoding a SpyCas9 nickase (nCas9) and the other encoding a DNA polymerase, that are delivered to cells in trans with the template guide RNA. Accordingly, in this Example, the editing efficiency of delivering mRNA encoding nCas9 and mRNA encoding a DNA polymerase in trans, was compared to that of mRNAs encoding fusion proteins nCas9-T5pol and nCas9-polK(GE411-412RV). As such, mRNAs encoding DNA-dependent DNA polymerases were delivered to Huh7 cells in trans with mRNA encoding a H840A nicking SpyCas9 (nCas9), a template guide RNA (tgRNA), and a nicking guide RNA (ngRNA).

The assessed DNA polymerases included polymerase theta (amino acids 1819-2590) referred to herein as PolQ(1819-2590) (SEQ ID NO: 1077), Klenow fragment exo-(D355A, E357A) referred to herein as Klenow exo-(D355A, E357A) (SEQ ID NO: 1105), Phi29 polymerase (with the following 11 mutations: M8R, D12A, E14A, V51A, M97T, G197D, E221K, Q497P, K512E, E515A, F526L) referred to herein as Phi29Pol (11M) (SEQ ID NO: 1185), polymerase Nu referred to herein as PolN (SEQ ID NO: 1089), or T5 polymerase (D164A, E166A) referred to herein as T5Pol(D164A,E166A) (SEQ ID NO: 1117). Each DNA polymerase was were delivered to Huh7 cells in trans with mRNA encoding a H840A nicking SpyCas9 (nCas9) (SEQ ID NO: 1010), a template guide RNA (tgRNA), and a nicking guide RNA (ngRNA).

Huh7 cells were prepared as described in Example 1, and at a density of 15,000 cells/well. After 24 hours of incubation at 37° C., 5% CO₂, cells were transfected using Lipofectamine™ MessengerMAX™ Reagent (Invitrogen, Cat. LMRNA015) according to manufacturer's instructions with mRNA(s) encoding nCas9 and DNA polymerase, in cis or in trans, and guide RNAs in a six-point, two-fold dilution series starting at a highest dose of 400 ng total RNA. The total RNA comprised mRNA (in a 1:1 molar ratio of nCas9 mRNA to DNA polymerase mRNA where delivered in trans) and guide RNAs in a 2:1 mRNA: guide RNA mass ratio. The guide RNA dose comprised tgRNA G032574 (with 3′ RNA extension; SEQ ID NO: 528) and ngRNA G025992 a 3:2 mass ratio of tgRNA:ngRNA. Samples were run in triplicate.

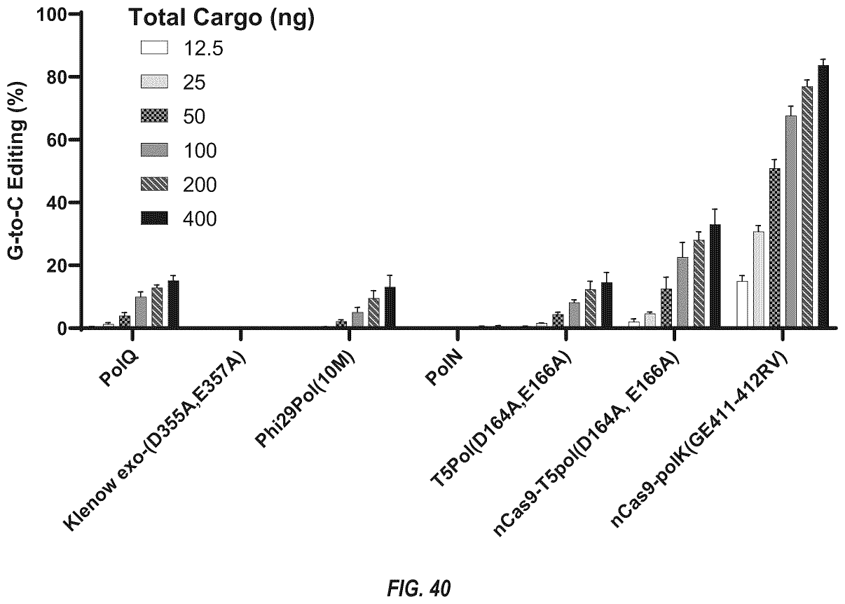

Table 46 and FIG. 40 show templated editing efficiency results.

TABLE 46
Templated editing at the HEK3 locus in Huh7 cells

| | | | Total | G-to-C | SD |
|---|---|---|---|---|---|
| | | Nicking | Cargo | Editing | (N = |
| mRNA ID | Guide ID | Guide ID | (ng) | (%) | 3) |
| PolQ(1819-2590) + | G032574 | G025992 | 400 | 15.1 | 1.7 |
| nCas9 in trans | G032574 | G025992 | 200 | 12.9 | 0.9 |
| (SEQ ID NOs: | G032574 | G025992 | 100 | 10.0 | 1.6 |
| 1077 and 1010) | G032574 | G025992 | 50 | 3.9 | 1.0 |
| | G032574 | G025992 | 25 | 1.3 | 0.5 |
| | G032574 | G025992 | 12.5 | 0.3 | 0.3 |
| Klenow exo- | G032574 | G025992 | 400 | 0.2 | 0.1 |
| (D355A, E357A) + | G032574 | G025992 | 200 | 0.2 | 0.1 |
| nCas9 in trans | G032574 | G025992 | 100 | 0.1 | 0.1 |
| (SEQ ID NOs: | G032574 | G025992 | 50 | 0.0 | 0.0 |
| 1105 and 1010) | G032574 | G025992 | 25 | 0.0 | 0.0 |
| | G032574 | G025992 | 12.5 | 0.0 | 0.0 |
| Phi29Pol(11M) + | G032574 | G025992 | 400 | 13.1 | 3.7 |
| nCas9 in trans | G032574 | G025992 | 200 | 9.6 | 2.4 |
| (SEQ ID NOs: | G032574 | G025992 | 100 | 5.1 | 1.5 |
| 1185 and 1010) | G032574 | G025992 | 50 | 2.1 | 0.6 |
| | G032574 | G025992 | 25 | 0.5 | 0.1 |
| | G032574 | G025992 | 12.5 | 0.2 | 0.1 |
| PolN + nCas9 in | G032574 | G025992 | 400 | 0.6 | 0.2 |
| trans (SEQ ID | G032574 | G025992 | 200 | 0.6 | 0.2 |
| NOs: 1089 and | G032574 | G025992 | 100 | 0.2 | 0.2 |
| 1010) | G032574 | G025992 | 50 | 0.1 | 0.1 |
| | G032574 | G025992 | 25 | 0.1 | 0.1 |
| | G032574 | G025992 | 12.5 | 0.0 | 0.0 |
| T5Pol(D164A, | G032574 | G025992 | 400 | 14.5 | 3.2 |
| E166A) + nCas9 in | G032574 | G025992 | 200 | 12.3 | 2.7 |
| trans (SEQ ID | G032574 | G025992 | 100 | 8.2 | 0.9 |
| Nos: 1117 and | G032574 | G025992 | 50 | 4.4 | 0.7 |
| 1010) | G032574 | G025992 | 25 | 1.6 | 0.1 |
| | G032574 | G025992 | 12.5 | 0.6 | 0.1 |
| nCas9- | G032574 | G025992 | 400 | 33.0 | 4.9 |
| T5pol(D164A, | G032574 | G025992 | 200 | 28.1 | 2.7 |
| E166A) fusion | G032574 | G025992 | 100 | 22.6 | 4.6 |
| (SEQ ID NO: | G032574 | G025992 | 50 | 12.5 | 3.7 |
| 1130) | G032574 | G025992 | 25 | 4.6 | 0.6 |
| | G032574 | G025992 | 12.5 | 2.0 | 1.0 |
| nCas9- | G032574 | G025992 | 400 | 83.7 | 2.0 |
| polK(GE411- | G032574 | G025992 | 200 | 77.0 | 2.1 |
| 412RV) fusion | G032574 | G025992 | 100 | 67.6 | 3.1 |
| (SEQ ID NO: | G032574 | G025992 | 50 | 50.9 | 2.8 |
| 1028) | G032574 | G025992 | 25 | 30.8 | 1.9 |
| | G032574 | G025992 | 12.5 | 15.0 | 1.8 |

### Example 18—DNA-Templated Editing in Huh7 Cells with Various DNA-Dependent DNA Polymerases (Polymerase K, Phi29 Polymerase, and T5 DNA Polymerase; Including Variants)

In this Example, templated DNA editing systems comprising (i) mRNA encoding a single polypeptide fusion (e.g., comprising nCas9 and DNA polymerase) and guide RNA components were designed and assessed for editing at the VEGFA locus in Huh7 cells. Editing efficiency at this target site was compared using mRNAs encoding fusion proteins each comprising a nicking SpyCas9 (nCas9) moiety and a different DNA-dependent DNA polymerase moiety (e.g., T5, PolK (GE411-412RV), PolK(1-526), PolK(1-526,GE411-412RV), and Phi29pol(11M)).

The following fusion proteins were screened: nCas9-T5pol(v2), nCas9-POLK(GE411-412RV), nCas9-POLK(1-526), nCas9-POLK(1-526,GE411-412RV), and nCas9-Phi29pol(11M). Each of these mRNAs was delivered to cells with a template guide RNA (tgRNA, G032595) comprising a spacer sequence targeting the VEGFA locus and a 3′ extension comprising a 12-nt DNA polymerase recruiting sequence (DRS) and an 18-nt DNA polymerase template encoding a 3-nucleotide substitution mutation (GAGG-to-CACT).

Huh7 cells were prepared as described in Example 1, and plated at a density of 15,000 cells/well. After 24 hours of incubation at 37° C., 5% CO₂, cells were transfected using Lipofectamine™ MessengerMAX™ Reagent (Invitrogen, Cat. LMRNA015) according to manufacturer's instructions with a mRNA encoding a nCas9-DNA polymerase fusion protein and a tgRNA (G032595) in a six-point, two-fold dilution series starting at a highest dose of 150 ng total RNA. The total RNA consisted of mRNA and tgRNA in a 2:1 mRNA:tgRNA mass ratio. Samples were run in triplicate.

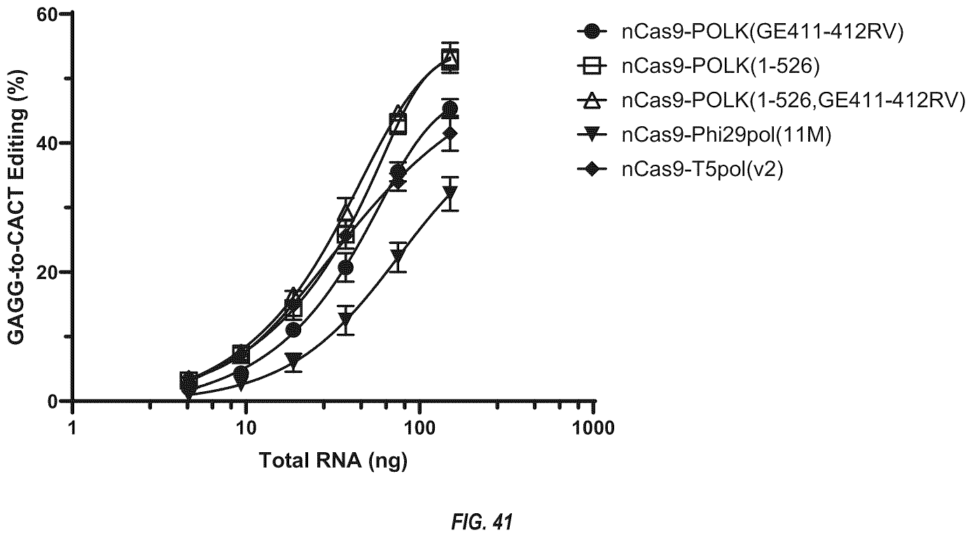

Cells were lysed 3 days post-treatment for NGS analysis as described in Example 1. Mean percent templated editing with standard deviation (SD) are shown in Table 47 and FIG. 41.

TABLE 47
Templated editing at the VEGFA locus in Huh7 cells

| | | Total RNA | Mean % | SD |
|---|---|---|---|---|
| mRNA ID | Guide ID | (ng) | Editing | (N = 3) |
| nCas9-POLK(GE411- | G032595 | 150 | 45.3% | 1.5% |
| 412RV) (SEQ ID NO: | G032595 | 75 | 35.5% | 1.5% |
| 1028) | G032595 | 37.5 | 20.7% | 2.2% |
| | G032595 | 18.8 | 11.0% | 0.6% |
| | G032595 | 9.4 | 4.3% | 0.6% |
| | G032595 | 4.7 | 2.0% | 0.3% |
| nCas9-POLK(1-526) | G032595 | 150 | 52.9% | 1.6% |
| (SEQ ID NO: 1227) | G032595 | 75 | 43.0% | 1.6% |
| | G032595 | 37.5 | 25.8% | 2.2% |
| | G032595 | 18.8 | 14.4% | 1.1% |
| | G032595 | 9.4 | 7.1% | 0.6% |
| | G032595 | 4.7 | 3.2% | 0.3% |
| nCas9-POLK(1-526, | G032595 | 150 | 53.2% | 2.3% |
| GE411-412RV) (SEQ | G032595 | 75 | 44.7% | 0.7% |
| ID NO: 1232) | G032595 | 37.5 | 29.3% | 2.2% |
| | G032595 | 18.8 | 16.5% | 0.7% |
| | G032595 | 9.4 | 7.6% | 0.5% |
| | G032595 | 4.7 | 3.5% | 0.2% |
| nCas9-Phi29pol(11M) | G032595 | 150 | 32.1% | 2.6% |
| (SEQ ID NO: 1237) | G032595 | 75 | 22.3% | 2.3% |
| | G032595 | 37.5 | 12.5% | 2.3% |
| | G032595 | 18.8 | 6.0% | 1.4% |
| | G032595 | 9.4 | 2.6% | 0.7% |
| | G032595 | 4.7 | 1.0% | 0.2% |
| nCas9-T5pol(v2) | G032595 | 150 | 41.5% | 2.7% |
| (SEQ ID NO: 1217) | G032595 | 75 | 33.9% | 1.4% |
| | G032595 | 37.5 | 25.6% | 1.2% |
| | G032595 | 18.8 | 14.8% | 2.2% |
| | G032595 | 9.4 | 7.3% | 1.3% |
| | G032595 | 4.7 | 3.6% | 0.4% |

### Example 19—Dose Response Experiment with VEGFA Guide RNAs in Primary Human Hepatocytes with Various DNA-Dependent DNA Polymerases (T5 DNA Polymerase, Polymerase K, and Phi29 DNA Polymerase; Including Variants)

In this Example, templated editing was assessed in primary human hepatocytes at an exemplary target locus (VEGFA). mRNAs encoding fusion proteins comprising a SpyCas9 nickase (nCas9) moiety and a DNA polymerase moiety were delivered to primary human hepatocytes (PHH) with a template guide RNA (tgRNA, G032595) encoding a 3-nt substitution.

In particular, the tgRNA comprised (i) a spacer sequence targeting the VEGFA locus and (ii) a 3′ extension sequence comprising a DNA polymerase recruitment site (i.e., a DNA polymerase recruiting sequence; DRS) and a DNA polymerase template encoding a GAGG-to-CACT 3-nt substitution mutation. Each mRNA encoded one of the following fusion proteins: nCas9-T5pol(v2), nCas9-POLK(GE411-412RV), nCas9-POLK(1-526), nCas9-POLK(1-526,GE411-412RV), and nCas9-Phi29pol(11M).

Primary human hepatocytes were prepared as described in Example 1, and plated at a density of 40,000 cells/well. After 24 hours of incubation at 37° C., 5% CO₂, cells were lipofected with mRNA and tgRNA (G032595) as described in Example 1. Guide nucleic acids and mRNAs were prepared in separate lipoplexes before mixing and diluting in a seven-point, two-fold dilution series. The highest dose was 300 ng total RNA in a 2:1 mRNA:tgRNA mass ratio. Samples were run in triplicate.

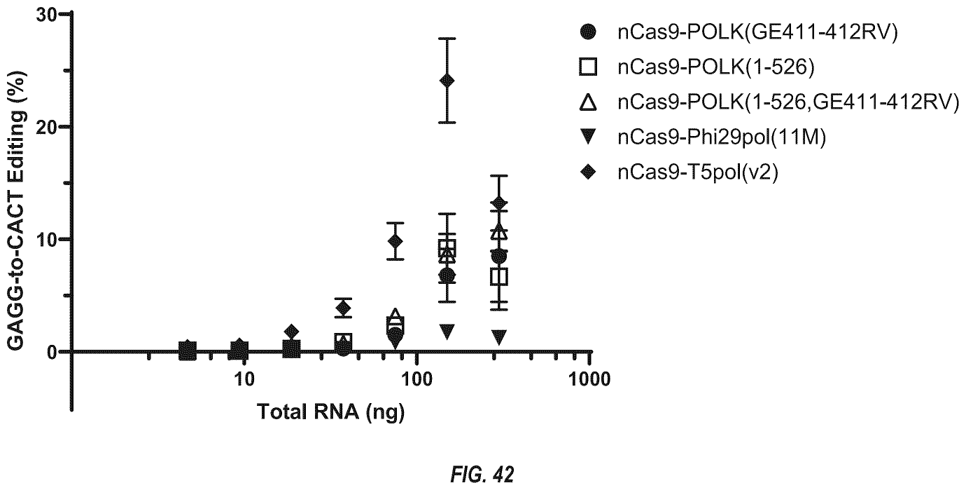

Cells were lysed 3 days post-treatment for NGS analysis as described in Example 1. Mean percent templated editing with standard deviation (SD) are shown in Table 48 and FIG. 42.

TABLE 48
Templated editing at the VEGFA locus
in primary human hepatocytes

| | | Total | | SD |
|---|---|---|---|---|
| | Guide | RNA | Mean % | (N = |
| mRNA ID | ID | (ng) | Editing | 3) |
| nCas9-POLK(GE411-412RV) | G032595 | 300 | 8.5% | 4.8% |
| (SEQ ID NO: 1028) | G032595 | 150 | 6.8% | 2.4% |
| | G032595 | 75 | 1.5% | 0.2% |
| | G032595 | 37.5 | 0.3% | 0.1% |
| | G032595 | 18.8 | 0.2% | 0.1% |
| | G032595 | 9.4 | 0.1% | 0.1% |
| | G032595 | 4.7 | 0.1% | 0.0% |
| nCas9-POLK(1-526) | G032595 | 300 | 6.7% | 2.2% |
| (SEQ ID NO: 1227) | G032595 | 150 | 9.2% | 3.1% |
| | G032595 | 75 | 2.3% | 0.1% |
| | G032595 | 37.5 | 0.9% | 0.3% |
| | G032595 | 18.8 | 0.3% | 0.2% |
| | G032595 | 9.4 | 0.1% | 0.0% |
| | G032595 | 4.7 | 0.1% | 0.1% |
| nCas9-POLK(1-526, | G032595 | 300 | 10.7% | 1.8% |
| GE411-412RV) | G032595 | 150 | 8.7% | 1.8% |
| (SEQ ID NO: 1232) | G032595 | 75 | 3.2% | 0.3% |
| | G032595 | 37.5 | 0.7% | 0.2% |
| | G032595 | 18.8 | 0.5% | 0.3% |
| | G032595 | 9.4 | 0.3% | 0.2% |
| | G032595 | 4.7 | 0.0% | 0.1% |
| nCas9-Phi29pol(11M) | G032595 | 300 | 1.2% | 0.4% |
| (SEQ ID NO: 1237) | G032595 | 150 | 1.7% | 0.4% |
| | G032595 | 75 | 0.8% | 0.5% |
| | G032595 | 37.5 | 0.3% | 0.2% |
| | G032595 | 18.8 | 0.1% | 0.2% |
| | G032595 | 9.4 | 0.1% | 0.1% |
| | G032595 | 4.7 | 0.0% | 0.0% |
| nCas9-T5pol(v2) | G032595 | 300 | 13.2% | 2.4% |
| (SEQ ID NO: 1217) | G032595 | 150 | 24.1% | 3.7% |
| | G032595 | 75 | 9.8% | 1.6% |
| | G032595 | 37.5 | 3.9% | 0.8% |
| | G032595 | 18.8 | 1.8% | 0.2% |
| | G032595 | 9.4 | 0.6% | 0.3% |
| | G032595 | 4.7 | 0.4% | 0.1% |

### Example 20—DNA Templated Editing in Huh7 Cells Using HEK3 Guide and MS2-Recruited Template With Various DNA-Dependent DNA Polymerases (T5 DNA Polymerase and Polymerase K; Including Variants)

This Example evaluated the ability of an aptamer in the 3′ extension region of a tgRNA to mediate templated editing via a corresponding 3′ extension-recruiting domain on a DNA-dependent DNA polymerase. Further, this Example evaluated whether such an aptamer could mediate templated editing following delivery in trans of an mRNA encoding a SpyCas9 nickase and an mRNA encoding a DNA polymerase (i.e., fused to a 3′ extension recruiting domain capable of recognizing the aptamer in the 3′ extension region of the tgRNA). Here, an MS2-loop aptamer linked to the 3′ extension region of a tgRNA was evaluated as an exemplary affinity tag for recruiting a corresponding MS2-coat protein (MCP) fused to a DNA polymerase.

A panel of mRNAs, each encoding one of the following DNA polymerases fused to MS2-coat protein (MCP) at the N-terminus, was screened for editing efficiency: T5pol(exo-), T5pol(exo-, A593R), POLK, POLK(GE411-412RV). MCP-DNA polymerase fusions were transfected into Huh7 cells along with an mRNA encoding a nicking SpyCas9 (nCas9) (SEQ ID NO: 1010). An mRNA encoding a fusion protein comprising a MCP region, a Polymerase K moiety and a nCas9 moiety (MCP-POLK-nCas9) was also screened. Guide nucleic acid components were designed for G-to-C editing at the HEK3 genomic locus. Three separate components were used: 1) a guide RNA (sgRNA) targeting the HEK3 locus, 2) an MS2-loop-linked 3′ extension (comprising a MS2 loop, a template sequence, and a DRS sequence) encoding a G-to-C edit at the sgRNA target site, and 3) a nicking guide (ngRNA). A panel of MS2-loop-linked 3′ extensions was designed in which the length of the template region was 15nt and the length of the DRS region was varied from 6- to 17-nt.

Huh7 cells were prepared as described in Example 1, plated at a density of 15,000 cells/well in a 96-well plate format and incubated at 37° C. and 5% CO₂ atmosphere for 24 hours. Nucleic acid components were delivered using Lipofectamine™ MessengerMAX™ Reagent (Thermo Fisher, Cat. LMRNA001) according to the manufacturer's protocol. Each well of Huh7 cells was transfected with 150 ng mRNA, 15 nM sgRNA (G023019), 15 nM MS2-loop-linked 3′ extension, and 10 nM ngRNA (G025992). For conditions where two mRNA molecules were delivered, a 1:1 molar ratio of the two mRNAs was applied. Samples were run in triplicate.

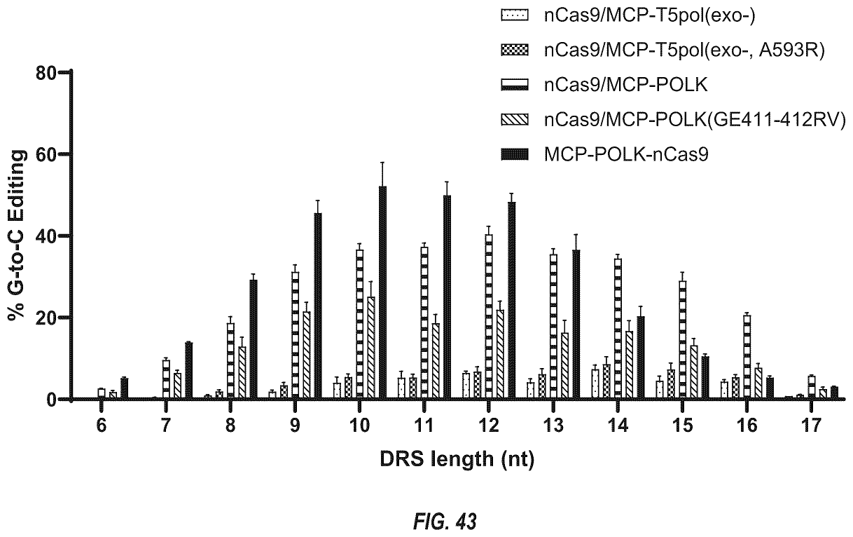

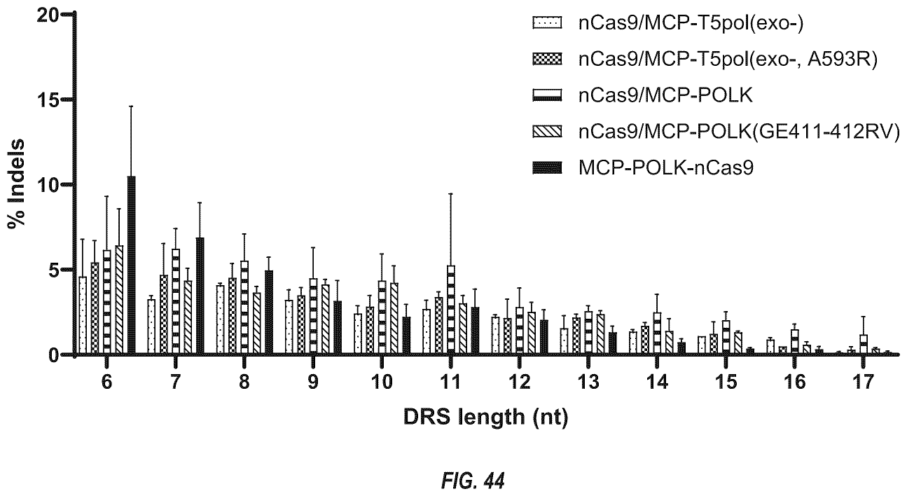

Cells were lysed 3 days post-treatment for NGS analysis as described in Example 1 to measure editing at the HEK3 locus, as well as byproduct indels generated at the HEK3 locus. Mean percent templated editing with standard deviation (SD) are shown in Table 49 and FIG. 43. Mean percent indels with standard deviation (SD) are shown in Table 50 and FIG. 44.

TABLE 49
Templated G-to-C editing at the HEK3 locus in Huh7 cells

| | | MS2- | | Mean | |
|---|---|---|---|---|---|
| | | loop- | DRS | % G- | SD |
| | | linked 3′ | length | to-C | (N = |
| mRNA 1 | mRNA 2 | extension | (nt) | Editing | 3) |
| nCas9 | MCP- | ST036312 | 6 | 0.1% | 0.1% |
| (SEQ ID NO: | T5pol(exo-) | ST036313 | 7 | 0.1% | 0.1% |
| 1010) | (SEQ ID NO: | ST036314 | 8 | 1.0% | 0.2% |
| | 1173) | ST036315 | 9 | 1.9% | 0.4% |
| | | ST034751 | 10 | 4.1% | 1.4% |
| | | ST036316 | 11 | 5.3% | 1.5% |
| | | ST036317 | 12 | 6.5% | 0.4% |
| | | ST036318 | 13 | 4.2% | 0.8% |
| | | ST036319 | 14 | 7.4% | 1.1% |
| | | ST036320 | 15 | 4.6% | 1.1% |
| | | ST036321 | 16 | 4.3% | 0.5% |
| | | ST036322 | 17 | 0.8% | 0.0% |
| nCas9 | MCP- | ST036312 | 6 | 0.0% | 0.0% |
| (SEQ ID NO: | T5pol(exo-, | ST036313 | 7 | 0.5% | 0.1% |
| 1010) | A593R) (SEQ | ST036314 | 8 | 1.9% | 0.4% |
| | ID NO: | ST036315 | 9 | 3.4% | 0.7% |
| | 1178) | ST034751 | 10 | 5.5% | 0.8% |
| | | ST036316 | 11 | 5.4% | 0.8% |
| | | ST036317 | 12 | 6.8% | 1.2% |
| | | ST036318 | 13 | 6.2% | 1.3% |
| | | ST036319 | 14 | 8.6% | 1.9% |
| | | ST036320 | 15 | 7.3% | 1.5% |
| | | ST036321 | 16 | 5.4% | 0.6% |
| | | ST036322 | 17 | 1.1% | 0.1% |
| nCas9 | MCP-POLK | ST036312 | 6 | 2.6% | 0.1% |
| (SEQ ID NO: | (SEQ ID NO: | ST036313 | 7 | 9.6% | 0.5% |
| 1010) | 1060) | ST036314 | 8 | 18.7% | 1.6% |
| | | ST036315 | 9 | 31.2% | 1.7% |
| | | ST034751 | 10 | 36.7% | 1.5% |
| | | ST036316 | 11 | 37.4% | 0.9% |
| | | ST036317 | 12 | 40.4% | 2.0% |
| | | ST036318 | 13 | 35.5% | 1.3% |
| | | ST036319 | 14 | 34.5% | 1.0% |
| | | ST036320 | 15 | 29.0% | 2.1% |
| | | ST036321 | 16 | 20.6% | 0.6% |
| | | ST036322 | 17 | 5.7% | 0.2% |
| nCas9 | MCP- | ST036312 | 6 | 1.8% | 0.3% |
| (SEQ ID NO: | POLK(GE411- | ST036313 | 7 | 6.5% | 0.7% |
| 1010) | 412RV) | ST036314 | 8 | 12.9% | 2.3% |
| | (SEQ ID NO: | ST036315 | 9 | 21.5% | 2.3% |
| | 1065) | ST034751 | 10 | 25.2% | 3.7% |
| | | ST036316 | 11 | 18.6% | 2.1% |
| | | ST036317 | 12 | 21.9% | 2.1% |
| | | ST036318 | 13 | 16.4% | 3.0% |
| | | ST036319 | 14 | 16.7% | 2.6% |
| | | ST036320 | 15 | 13.3% | 1.7% |
| | | ST036321 | 16 | 7.8% | 1.0% |
| | | ST036322 | 17 | 2.5% | 0.5% |
| MCP-POLK- | None | ST036312 | 6 | 5.2% | 0.3% |
| nCas9 | | ST036313 | 7 | 14.0% | 0.1% |
| (SEQ ID NO: | | ST036314 | 8 | 29.3% | 1.4% |
| 1057) | | ST036315 | 9 | 45.6% | 3.1% |
| | | ST034751 | 10 | 52.2% | 5.8% |
| | | ST036316 | 11 | 49.9% | 3.3% |
| | | ST036317 | 12 | 48.3% | 2.1% |
| | | ST036318 | 13 | 36.6% | 3.7% |
| | | ST036319 | 14 | 20.4% | 2.4% |
| | | ST036320 | 15 | 10.5% | 0.6% |
| | | ST036321 | 16 | 5.3% | 0.4% |
| | | ST036322 | 17 | 3.1% | 0.1% |

TABLE 50
Byproduct indels generated at the HEK3 locus in Huh7 cells

| | | | DRS | Mean | SD |
|---|---|---|---|---|---|
| | | Guide | length | % | (N = |
| mRNA 1 | mRNA 2 | ID | (nt) | Indels | 3) |
| nCas9 | MCP-T5pol(exo-) | ST036312 | 6 | 4.6% | 2.2% |
| (SEQ ID | (SEQ ID NO: 1173) | ST036313 | 7 | 3.3% | 0.2% |
| NO: 1010) | | ST036314 | 8 | 4.1% | 0.1% |
| | | ST036315 | 9 | 3.2% | 0.6% |
| | | ST034751 | 10 | 2.4% | 0.5% |
| | | ST036316 | 11 | 2.7% | 0.5% |
| | | ST036317 | 12 | 2.2% | 0.1% |
| | | ST036318 | 13 | 1.6% | 0.7% |
| | | ST036319 | 14 | 1.4% | 0.1% |
| | | ST036320 | 15 | 1.1% | 0.0% |
| | | ST036321 | 16 | 0.9% | 0.1% |
| | | ST036322 | 17 | 0.1% | 0.1% |
| nCas9 | MCP-T5pol(exo-, | ST036312 | 6 | 5.4% | 1.3% |
| (SEQ ID | A593R) | ST036313 | 7 | 4.7% | 1.8% |
| NO: 1010) | (SEQ ID NO: 1178) | ST036314 | 8 | 4.5% | 0.8% |
| | | ST036315 | 9 | 3.5% | 0.5% |
| | | ST034751 | 10 | 2.8% | 0.7% |
| | | ST036316 | 11 | 3.4% | 0.3% |
| | | ST036317 | 12 | 2.2% | 1.1% |
| | | ST036318 | 13 | 2.2% | 0.2% |
| | | ST036319 | 14 | 1.7% | 0.2% |
| | | ST036320 | 15 | 1.2% | 0.7% |
| | | ST036321 | 16 | 0.5% | 0.0% |
| | | ST036322 | 17 | 0.3% | 0.2% |
| nCas9 | MCP-POLK | ST036312 | 6 | 6.2% | 3.2% |
| (SEQ ID | (SEQ ID NO: 1060) | ST036313 | 7 | 6.2% | 1.2% |
| NO: 1010) | | ST036314 | 8 | 5.5% | 1.6% |
| | | ST036315 | 9 | 4.5% | 1.8% |
| | | ST034751 | 10 | 4.4% | 1.6% |
| | | ST036316 | 11 | 5.3% | 4.2% |
| | | ST036317 | 12 | 2.8% | 1.1% |
| | | ST036318 | 13 | 2.6% | 0.3% |
| | | ST036319 | 14 | 2.5% | 1.1% |
| | | ST036320 | 15 | 2.0% | 0.5% |
| | | ST036321 | 16 | 1.5% | 0.3% |
| | | ST036322 | 17 | 1.2% | 1.0% |
| nCas9 | MCP-POLK(GE411- | ST036312 | 6 | 6.4% | 2.1% |
| (SEQ ID | 412RV) | ST036313 | 7 | 4.4% | 0.7% |
| NO: 1010) | (SEQ ID NO: 1065) | ST036314 | 8 | 3.7% | 0.4% |
| | | ST036315 | 9 | 4.1% | 0.3% |
| | | ST034751 | 10 | 4.2% | 1.0% |
| | | ST036316 | 11 | 3.0% | 0.5% |
| | | ST036317 | 12 | 2.5% | 0.6% |
| | | ST036318 | 13 | 2.4% | 0.2% |
| | | ST036319 | 14 | 1.4% | 0.7% |
| | | ST036320 | 15 | 1.3% | 0.1% |
| | | ST036321 | 16 | 0.6% | 0.2% |
| | | ST036322 | 17 | 0.4% | 0.1% |
| MCP- | None | ST036312 | 6 | 10.5% | 4.1% |
| POLK- | | ST036313 | 7 | 6.9% | 2.0% |
| nCas9 | | ST036314 | 8 | 5.0% | 0.8% |
| (SEQ ID | | ST036315 | 9 | 3.2% | 1.2% |
| NO: 1057) | | ST034751 | 10 | 2.2% | 0.7% |
| | | ST036316 | 11 | 2.8% | 1.1% |
| | | ST036317 | 12 | 2.1% | 0.6% |
| | | ST036318 | 13 | 1.3% | 0.4% |
| | | ST036319 | 14 | 0.7% | 0.2% |
| | | ST036320 | 15 | 0.4% | 0.1% |
| | | ST036321 | 16 | 0.3% | 0.2% |
| | | ST036322 | 17 | 0.2% | 0.1% |

### Example 21—Multiple Edit Types at VEGFA Locus in Huh7 Cells with Various Types and Positions of Base Edits, and Template Lengths for Various Length Insertions or Deletions

This Example assessed (a) the editing efficiency of different 3′ extensions with an aptamer (MS2-loop-linked 3′ extensions) encoding various templated edits at different distances from the DRS/template junction; (b) the editing efficiency of various template lengths to direct deletions and insertions of different lengths; and (c) the effect on editing efficiency of various numbers and positions of aptamers (MS2-loops within the MS2-loop-linked 3′ extension). Indel analysis and editing analysis were performed using guide RNAs (gRNAs) targeting the VEGFA locus. These gRNAs included the same sgRNA (G034491) paired with an MS2-loop-linked 3′ extension comprising a DNA template sequence and a DRS sequence.

To assess the editing efficiency of different MS2-loop-linked 3′ extensions encoding various templated edits at different distances from the DRS/template junction, MS2-loop-linked 3′ extensions encoding either a transition or a transversion at different positions of the MS2-loop-linked 3′ extension, up to 18 nucleotides away from the DRS/template junction in the 3′ to 5′ direction, were generated. The gRNAs and MS2-loop-linked 3′ extensions were delivered to Huh7 cells with either a SpyCas9 encoding mRNA (SEQ ID NO: 1001) to observe indels resulting from SpyCas9 cleavage activity, or an mRNA encoding an MCP-PolK-nCas9 fusion protein (SEQ ID NO: 1057) to observe templated editing efficiency.

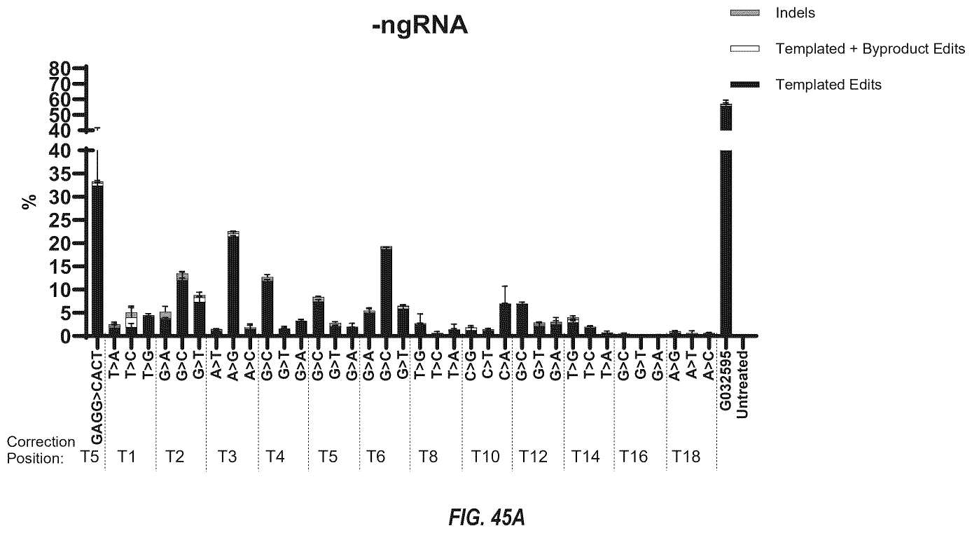

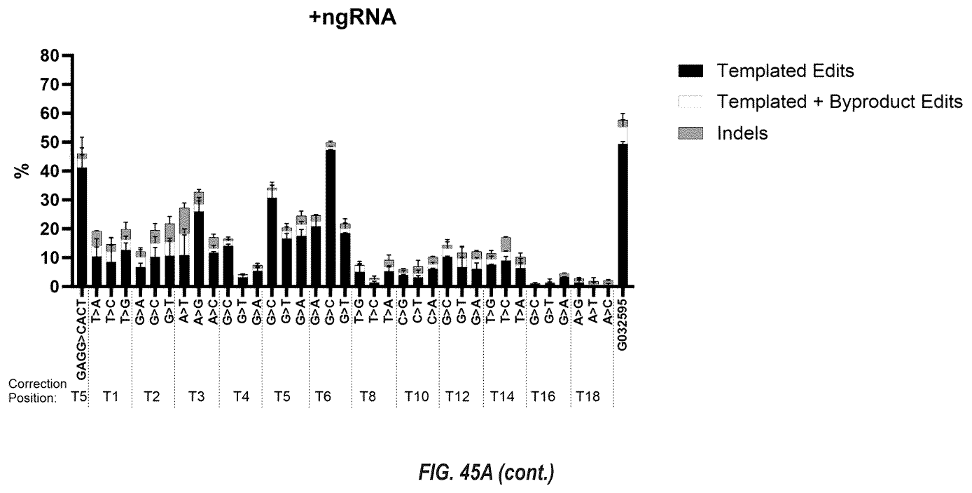

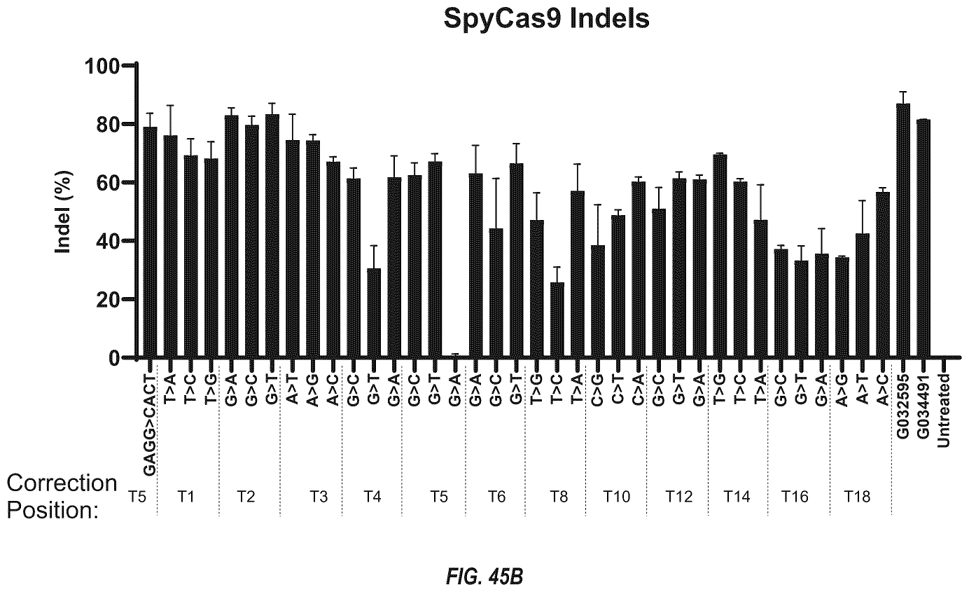

Huh7 cells were prepared as described in Example 1, and plated at a density of 15,000 cells/well. After 24 hours of incubation at 37° C., 5% CO₂, cells were transfected using Lipofectamine™ MessengerMAX™ Reagent (Invitrogen, Cat. LMRNA015) according to manufacturer's instructions with 300 ng of mRNA, sgRNA (G034491) at a final concentration of 25 nM, and either a MS2-loop-linked 3′ extension at a final concentration of 25 nM, or a MS2-loop-linked 3′ extension and a ngRNA at a 3:2 molar ratio respectively (15 nM:10 nM). Control groups treated with only tgRNA (G032595) or sgRNA (G034491) (i.e., no MS2-loop-linked 3′ extension) were transfected with 300 ng of mRNA and a final concentration of 25 nM of gRNA. Samples were prepared in duplicate. After transfection and incubation at 37° C., 5% CO₂ for 72 hours, samples were prepared for next-generation sequencing (NGS) and analysis for editing efficiency as described in Example 1. Table 51A and FIG. 45A show the editing data at the VEGFA locus using MCP-PolK-nCas9. Three measurements are shown: the “templated edits” corresponding to reads with the point correction as encoded in the template sequence of the corresponding MS2-loop-linked 3′ extension, the “templated+byproduct edits” corresponding to reads where the template-encoded point correction is observed along with byproduct edit(s) or indel(s) at or near the target site of the tgRNA, and the “indels” corresponding to reads where indels are observed at or near the target site of the tgRNA, as described in Example 1. Table 51B and FIG. 45B show the indel data at the VEGFA locus using SpyCas9.

TABLE 51A
Templated editing at VEGFA locus using MCP-PolK-nCas9 in Huh7 cells.

| | | | | | | | | | SD of | | |
|---|---|---|---|---|---|---|---|---|---|---|---|
| | | | | | | | | Mean % | Templated | | |
| | | | | | | Mean | SD of | Templated | + | | |
| | | | MS2-loop- | Correction | | % | Templated | + | Byproduct | Mean | SD of |
| mRNA | ngRNA | Guide | linked 3' | Position in | | Templated | Edits | Byproduct | Edits | % | Indels |
| ID | ID | ID | extension | Template | Edit Type | Editing | (N = 2) | Edits | (N = 2) | Indels | (N = 2) |
| MCP- | N/A | G034491 | ST036353 | T5 | GAGG > CACT | 32.45 | 9.26 | 0.50 | 0.14 | 0.35 | 0.21 |
| PolK- | | (SEQ ID | (SEQ ID | | | | | | | | |
| nCas9 | | NO: 482) | NO: 713) | | | | | | | | |
| (SEQ ID | | | ST036359 | T1 | T > A | 1.85 | 0.78 | 0.30 | 0.28 | 0.35 | 0.49 |
| NO: | | | (SEQ ID | | | | | | | | |
| 1057) | | | NO: 719) | | | | | | | | |
| | | | ST036360 | T1 | T > C | 1.95 | 0.78 | 2.05 | 2.47 | 1.05 | 1.06 |
| | | | (SEQ ID | | | | | | | | |
| | | | NO: 720) | | | | | | | | |
| | | | ST036361 | T1 | T > G | 4.20 | 0.14 | 0.05 | 0.07 | 0.25 | 0.35 |
| | | | (SEQ ID | | | | | | | | |
| | | | NO: 721) | | | | | | | | |
| | | | ST036362 | T2 | G > A | 3.70 | 0.28 | 0.15 | 0.07 | 1.35 | 1.20 |
| | | | (SEQ ID | | | | | | | | |
| | | | NO: 722) | | | | | | | | |
| | | | ST036363 | T2 | G > C | 12.10 | 1.84 | 0.30 | 0.14 | 1.10 | 0.28 |
| | | | (SEQ ID | | | | | | | | |
| | | | NO: 723) | | | | | | | | |
| | | | ST036364 | T2 | G > T | 7.40 | 1.13 | 0.95 | 0.07 | 0.45 | 0.64 |
| | | | (SEQ ID | | | | | | | | |
| | | | NO: 724) | | | | | | | | |
| | | | ST036365 | T3 | A > T | 1.15 | 0.07 | 0.20 | 0.28 | 0.15 | 0.07 |
| | | | (SEQ ID | | | | | | | | |
| | | | NO: 725) | | | | | | | | |
| | | | ST036366 | T3 | A > G | 21.45 | 0.21 | 0.65 | 0.35 | 0.45 | 0.07 |
| | | | (SEQ ID | | | | | | | | |
| | | | NO: 726) | | | | | | | | |
| | | | ST036367 | T3 | A > C | 1.50 | 1.13 | 0.00 | 0.00 | 0.35 | 0.49 |
| | | | (SEQ ID | | | | | | | | |
| | | | NO: 727) | | | | | | | | |
| | | | ST036368 | T4 | G > C | 11.85 | 0.35 | 0.25 | 0.07 | 0.60 | 0.57 |
| | | | (SEQ ID | | | | | | | | |
| | | | NO: 728) | | | | | | | | |
| | | | ST036369 | T4 | G > T | 1.40 | 0.71 | 0.10 | 0.14 | 0.10 | 0.14 |
| | | | (SEQ ID | | | | | | | | |
| | | | NO: 729) | | | | | | | | |
| | | | ST036370 | T4 | G > A | 3.15 | 0.49 | 0.00 | 0.00 | 0.15 | 0.21 |
| | | | (SEQ ID | | | | | | | | |
| | | | NO: 730) | | | | | | | | |
| | | | ST036371 | T5 | G > C | 7.35 | 0.49 | 0.10 | 0.00 | 1.00 | 0.14 |
| | | | (SEQ ID | | | | | | | | |
| | | | NO: 731) | | | | | | | | |
| | | | ST036372 | T5 | G > T | 1.95 | 0.21 | 0.15 | 0.21 | 0.70 | 0.28 |
| | | | (SEQ ID | | | | | | | | |
| | | | NO: 732) | | | | | | | | |
| | | | ST036373 | T5 | G > A | 1.85 | 0.92 | 0.05 | 0.07 | 0.10 | 0.00 |
| | | | (SEQ ID | | | | | | | | |
| | | | NO: 733) | | | | | | | | |
| | | | ST036374 | T6 | G > A | 4.90 | 0.99 | 0.10 | 0.00 | 0.45 | 0.64 |
| | | | (SEQ ID | | | | | | | | |
| | | | NO: 734) | | | | | | | | |
| | | | ST036375 | T6 | G > C | 18.75 | 0.35 | 0.30 | 0.00 | 0.30 | 0.00 |
| | | | (SEQ ID | | | | | | | | |
| | | | NO: 735) | | | | | | | | |
| | | | ST036376 | T6 | G > T | 5.95 | 0.78 | 0.40 | 0.42 | 0.15 | 0.07 |
| | | | (SEQ ID | | | | | | | | |
| | | | NO: 736) | | | | | | | | |
| | | | ST036377 | T8 | T > G | 2.45 | 2.33 | 0.05 | 0.07 | 0.25 | 0.07 |
| | | | (SEQ ID | | | | | | | | |
| | | | NO: 737) | | | | | | | | |
| | | | ST036378 | T8 | T > C | 0.65 | 0.35 | 0.00 | 0.00 | 0.00 | 0.00 |
| | | | (SEQ ID | | | | | | | | |
| | | | NO: 738) | | | | | | | | |
| | | | ST036379 | T8 | T > A | 1.25 | 1.34 | 0.00 | 0.00 | 0.15 | 0.21 |
| | | | (SEQ ID | | | | | | | | |
| | | | NO: 739) | | | | | | | | |
| | | | ST036380 | T10 | C > G | 1.25 | 0.64 | 0.55 | 0.49 | 0.05 | 0.07 |
| | | | (SEQ ID | | | | | | | | |
| | | | NO: 740) | | | | | | | | |
| | | | ST036381 | T10 | C > T | 1.20 | 0.14 | 0.00 | 0.00 | 0.25 | 0.21 |
| | | | (SEQ ID | | | | | | | | |
| | | | NO: 741) | | | | | | | | |
| | | | ST036382 | T10 | C > A | 6.85 | 3.89 | 0.10 | 0.00 | 0.05 | 0.07 |
| | | | (SEQ ID | | | | | | | | |
| | | | NO: 742) | | | | | | | | |
| | | | ST036383 | T12 | G > C | 6.90 | 0.42 | 0.10 | 0.00 | 0.00 | 0.00 |
| | | | (SEQ ID | | | | | | | | |
| | | | NO: 743) | | | | | | | | |
| | | | ST036384 | T12 | G > T | 2.20 | 0.85 | 0.25 | 0.35 | 0.45 | 0.07 |
| | | | (SEQ ID | | | | | | | | |
| | | | NO: 744) | | | | | | | | |
| | | | ST036385 | T12 | G > A | 2.55 | 1.48 | 0.30 | 0.00 | 0.20 | 0.28 |
| | | | (SEQ ID | | | | | | | | |
| | | | NO: 745) | | | | | | | | |
| | | | ST036386 | T14 | T > G | 2.95 | 0.35 | 0.60 | 0.14 | 0.45 | 0.35 |
| | | | (SEQ ID | | | | | | | | |
| | | | NO: 746) | | | | | | | | |
| | | | ST036387 | T14 | T > C | 1.75 | 0.35 | 0.00 | 0.00 | 0.25 | 0.21 |
| | | | (SEQ ID | | | | | | | | |
| | | | NO: 747) | | | | | | | | |
| | | | ST036388 | T14 | T > A | 0.75 | 0.35 | 0.00 | 0.00 | 0.00 | 0.00 |
| | | | (SEQ ID | | | | | | | | |
| | | | NO: 748) | | | | | | | | |
| | | | ST036389 | T16 | G > C | 0.50 | 0.14 | 0.00 | 0.00 | 0.00 | 0.00 |
| | | | (SEQ ID | | | | | | | | |
| | | | NO: 749) | | | | | | | | |
| | | | ST036390 | T16 | G > T | 0.30 | 0.00 | 0.00 | 0.00 | 0.00 | 0.00 |
| | | | (SEQ ID | | | | | | | | |
| | | | NO: 750) | | | | | | | | |
| | | | ST036391 | T16 | G > A | 0.05 | 0.07 | 0.00 | 0.00 | 0.10 | 0.14 |
| | | | (SEQ ID | | | | | | | | |
| | | | NO: 751) | | | | | | | | |
| | | | ST036392 | T18 | A > G | 0.65 | 0.21 | 0.00 | 0.00 | 0.40 | 0.14 |
| | | | (SEQ ID | | | | | | | | |
| | | | NO: 752) | | | | | | | | |
| | | | ST036393 | T18 | A > T | 0.00 | 0.00 | 0.10 | 0.14 | 0.55 | 0.49 |
| | | | (SEQ ID | | | | | | | | |
| | | | NO: 753) | | | | | | | | |
| | | | ST036394 | T18 | A > C | 0.50 | 0.28 | 0.00 | 0.00 | 0.15 | 0.07 |
| | | | (SEQ ID | | | | | | | | |
| | | | NO: 754) | | | | | | | | |
| | | G032595 | N/A | T5 | GAGG > CACT | 55.75 | 3.75 | 0.90 | 0.14 | 0.45 | 0.64 |
| | | (SEQ ID | | | | | | | | | |
| | | NO: 526) | | | | | | | | | |
| | G034622 | G034491 | ST036353 | T5 | GAGG > CACT | 41.25 | 10.54 | 3.00 | 1.41 | 1.85 | 1.91 |
| | (SEQ ID | | ST036359 | T1 | T > A | 10.35 | 3.32 | 3.70 | 2.55 | 5.20 | 0.14 |
| | NO: 527) | | ST036360 | T1 | T > C | 8.50 | 5.80 | 3.60 | 4.95 | 2.50 | 2.26 |
| | | | ST036361 | T1 | T > G | 12.70 | 2.40 | 3.60 | 1.13 | 3.55 | 2.47 |
| | | | ST036362 | T2 | G > A | 6.70 | 1.41 | 3.50 | 2.55 | 2.05 | 1.20 |
| | | | ST036363 | T2 | G > C | 10.30 | 3.25 | 4.65 | 2.33 | 4.55 | 2.33 |
| | | | ST036364 | T2 | G > T | 10.60 | 5.23 | 4.85 | 1.34 | 6.35 | 2.47 |
| | | | ST036365 | T3 | A > T | 10.80 | 7.07 | 7.50 | 1.70 | 9.00 | 1.70 |
| | | | ST036366 | T3 | A > G | 26.00 | 4.81 | 2.50 | 1.13 | 4.30 | 0.85 |
| | | | ST036367 | T3 | A > C | 11.70 | 0.42 | 1.55 | 1.06 | 3.80 | 1.13 |
| | | | ST036368 | T4 | G > C | 14.05 | 0.64 | 1.75 | 0.49 | 0.80 | 0.57 |
| | | | ST036369 | T4 | G > T | 3.15 | 0.92 | 0.80 | 0.42 | 0.25 | 0.21 |
| | | | ST036370 | T4 | G > A | 5.35 | 1.20 | 0.95 | 0.64 | 1.15 | 0.64 |
| | | | ST036371 | T5 | G > C | 30.75 | 5.44 | 2.65 | 1.63 | 0.80 | 0.99 |
| | | | ST036372 | T5 | G > T | 16.65 | 1.77 | 2.55 | 2.62 | 1.20 | 0.28 |
| | | | ST036373 | T5 | G > A | 17.55 | 2.19 | 3.95 | 1.06 | 3.05 | 1.63 |
| | | | ST036374 | T6 | G > A | 20.80 | 3.96 | 1.85 | 2.33 | 1.95 | 0.07 |
| | | | ST036375 | T6 | G > C | 47.20 | 0.28 | 1.15 | 0.21 | 1.55 | 0.49 |
| | | | ST036376 | T6 | G > T | 18.45 | 0.21 | 1.65 | 1.91 | 1.70 | 1.70 |
| | | | ST036377 | T8 | T > G | 5.00 | 2.97 | 2.25 | 1.48 | 0.20 | 0.14 |
| | | | ST036378 | T8 | T > C | 1.45 | 0.35 | 0.90 | 1.27 | 0.55 | 0.78 |
| | | | ST036379 | T8 | T > A | 5.30 | 1.56 | 1.55 | 0.35 | 2.30 | 1.84 |
| | | | ST036380 | T10 | C > G | 4.05 | 0.21 | 0.75 | 0.78 | 1.25 | 0.21 |
| | | | ST036381 | T10 | C > T | 3.15 | 0.64 | 1.40 | 1.13 | 2.40 | 2.12 |
| | | | ST036382 | T10 | C > A | 6.20 | 0.14 | 1.60 | 0.57 | 2.40 | 0.28 |
| | | | ST036383 | T12 | G > C | 10.30 | 0.28 | 2.85 | 2.33 | 1.35 | 1.77 |
| | | | ST036384 | T12 | G > T | 6.75 | 3.32 | 3.05 | 3.89 | 1.90 | 2.26 |
| | | | ST036385 | T12 | G > A | 6.20 | 1.98 | 3.30 | 0.57 | 2.65 | 0.35 |
| | | | ST036386 | T14 | T > G | 7.55 | 0.21 | 1.95 | 1.06 | 1.95 | 1.06 |
| | | | ST036387 | T14 | T > C | 8.95 | 1.48 | 3.15 | 0.21 | 5.00 | 0.14 |
| | | | ST036388 | T14 | T > A | 6.35 | 3.32 | 1.25 | 0.64 | 2.60 | 1.41 |
| | | | ST036389 | T16 | G > C | 0.90 | 0.42 | 0.00 | 0.00 | 0.25 | 0.21 |
| | | | ST036390 | T16 | G > T | 1.10 | 1.56 | 0.00 | 0.00 | 0.35 | 0.21 |
| | | | ST036391 | T16 | G > A | 3.25 | 0.21 | 0.10 | 0.00 | 1.25 | 0.07 |
| | | | ST036392 | T18 | A > G | 1.40 | 1.27 | 0.35 | 0.49 | 1.05 | 0.35 |
| | | | ST036393 | T18 | A > T | 0.30 | 0.14 | 0.50 | 0.71 | 1.00 | 1.27 |
| | | | ST036394 | T18 | A > C | 0.25 | 0.07 | 0.35 | 0.49 | 1.40 | 0.42 |
| | | G032595 | N/A | T5 | N/A | 49.45 | 0.78 | 5.85 | 2.47 | 2.40 | 2.26 |
| Untreated | N/A | N/A | N/A | N/A | N/A | 0.00 | 0.00 | 0.00 | 0.00 | 0.00 | 0.00 |

TABLE 51B
Indel analysis at VEGFA locus using SpyCas9 in Huh7 cells.

| | | | MS2-loop- | | | Mean | |
|---|---|---|---|---|---|---|---|
| mRNA | ngRNA | Guide | linked 3' | Correction | Templated | % | SD |
| ID | ID | ID | extension | Position | Edit Type | Indels | (N = 2) |
| SpyCas9 | N/A | G034 | ST036353 | T5 | GAGG > CACT | 79.00 | 4.67 |
| (SEQ | | 491 | ST036359 | T1 | T > A | 76.15 | 10.25 |
| ID | | | ST036360 | T1 | T > C | 69.30 | 5.66 |
| NO: | | | ST036361 | T1 | T > G | 68.20 | 5.80 |
| 1001) | | | ST036362 | T2 | G > A | 83.00 | 2.55 |
| | | | ST036363 | T2 | G > C | 79.70 | 2.97 |
| | | | ST036364 | T2 | G > T | 83.35 | 3.75 |
| | | | ST036365 | T3 | A > T | 74.50 | 8.91 |
| | | | ST036366 | T3 | A > G | 74.35 | 2.05 |
| | | | ST036367 | T3 | A > C | 67.10 | 1.70 |
| | | | ST036368 | T4 | G > C | 61.40 | 3.54 |
| | | | ST036369 | T4 | G > T | 30.65 | 7.71 |
| | | | ST036370 | T4 | G > A | 61.80 | 7.35 |
| | | | ST036371 | T5 | G > C | 62.55 | 4.17 |
| | | | ST036372 | T5 | G > T | 67.20 | 2.69 |
| | | | ST036373 | T5 | G > A | 0.60 | 0.71 |
| | | | ST036374 | T6 | G > A | 63.15 | 9.55 |
| | | | ST036375 | T6 | G > C | 44.30 | 17.11 |
| | | | ST036376 | T6 | G > T | 66.50 | 6.79 |
| | | | ST036377 | T8 | T > G | 47.10 | 9.33 |
| | | | ST036378 | T8 | T > C | 25.75 | 5.30 |
| | | | ST036379 | T8 | T > A | 57.15 | 9.12 |
| | | | ST036380 | T10 | C > G | 38.50 | 13.86 |
| | | | ST036381 | T10 | C > T | 48.80 | 1.84 |
| | | | ST036382 | T10 | C > A | 60.30 | 1.56 |
| | | | ST036383 | T12 | G > C | 51.05 | 7.28 |
| | | | ST036384 | T12 | G > T | 61.45 | 2.19 |
| | | | ST036385 | T12 | G > A | 61.05 | 1.48 |
| | | | ST036386 | T14 | T > G | 69.65 | 0.35 |
| | | | ST036387 | T14 | T > C | 60.30 | 0.99 |
| | | | ST036388 | T14 | T > A | 47.20 | 12.02 |
| | | | ST036389 | T16 | G > C | 37.20 | 1.27 |
| | | | ST036390 | T16 | G > T | 33.25 | 5.02 |
| | | | ST036391 | T16 | G > A | 35.65 | 8.56 |
| | | | ST036392 | T18 | A > G | 34.40 | 0.42 |
| | | | ST036393 | T18 | A > T | 42.55 | 11.24 |
| | | | ST036394 | T18 | A > C | 56.80 | 1.41 |
| | | G032595 | N/A | T5 | N/A | 87.05 | 4.03 |
| | | G034491 | N/A | N/A | N/A | 81.55 | 0.07 |
| Untreated | | N/A | N/A | N/A | N/A | 0.00 | 0.00 |

To assess the editing efficiency of various template lengths within the MS2-loop-linked 3′ extensions to direct deletions and insertions of different lengths, MS2-loop-linked 3′ extensions with different template lengths were generated. Huh7 cells were prepared as described in Example 1, and plated at a density of 15,000 cells/well. After 24 hours, of incubation at 37° C., 5% CO₂, cells were transfected using Lipofectamine™ MessengerMAX™ Reagent (Invitrogen, Cat. LMRNA015) according to manufacturer's instructions with 300 ng of mRNA, sgRNA (G034491) at a final concentration of 25 nM, and either a MS2-loop-linked 3′ extension at a final concentration of 25 nM, or a MS2-loop-linked 3′ extension and a ngRNA at a 3:2 molar ratio respectively (15 nM:10 nM). Cells treated with only tgRNA (G032595) or sgRNA (G034491) (i.e., no MS2-loop-linked 3′ extension) were transfected with 300 ng of mRNA and a final concentration of 25 nM of gRNA. Samples were prepared in duplicate. After transfection and incubation at 37° C., 5% CO₂ for 72 hours, samples were prepared for next-generation sequencing (NGS) and analysis for editing efficiency as described in Example 1.

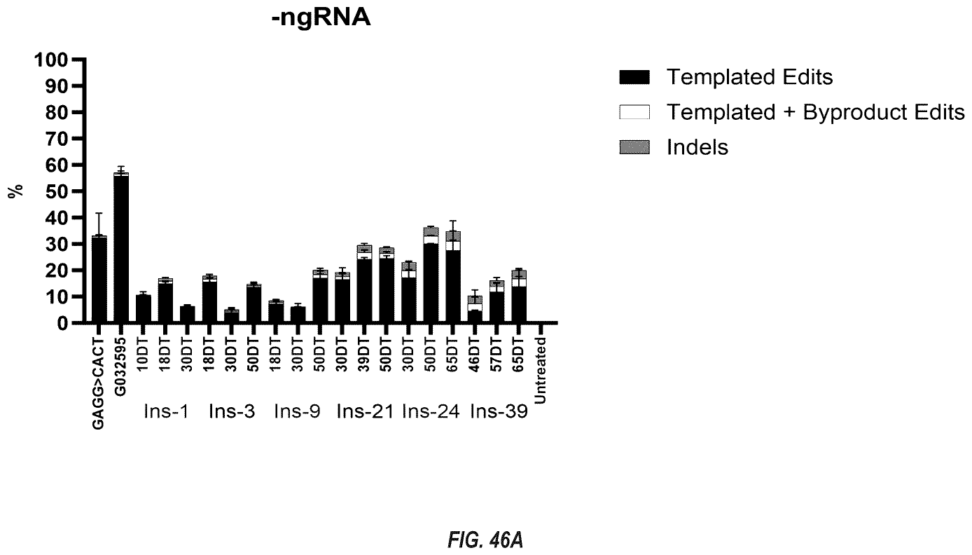

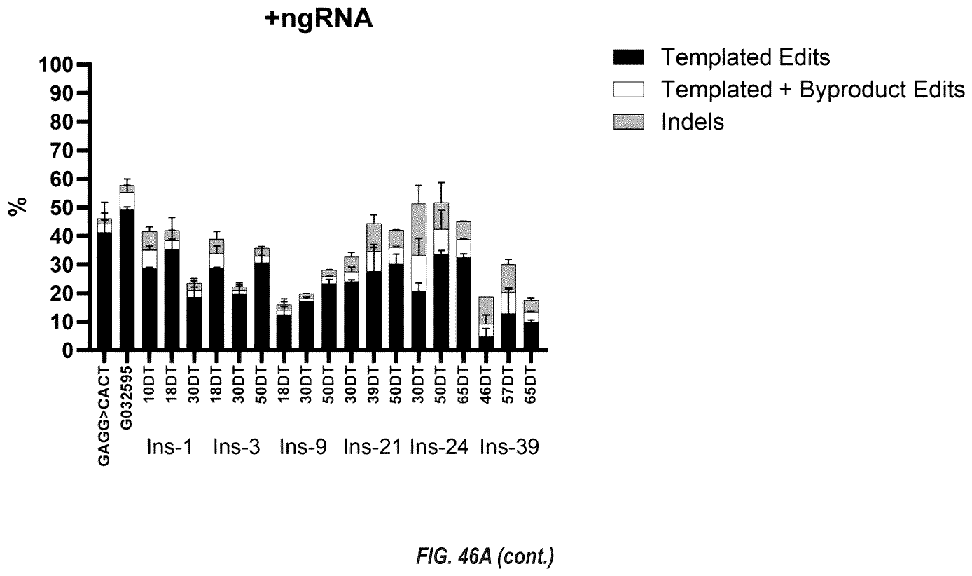

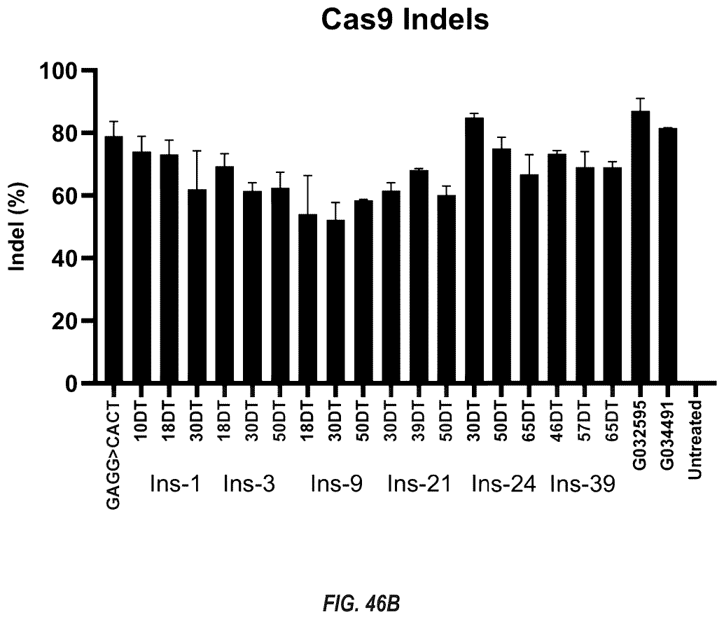

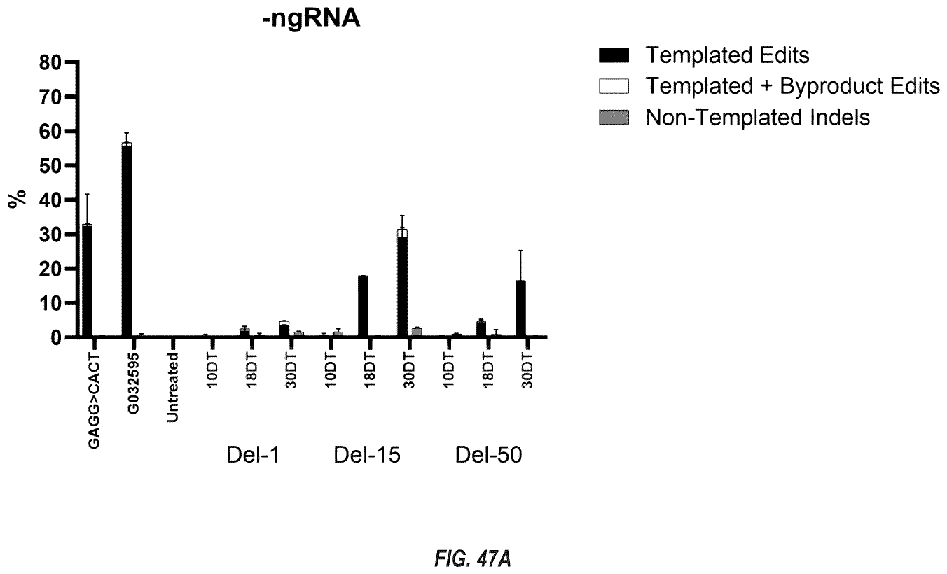

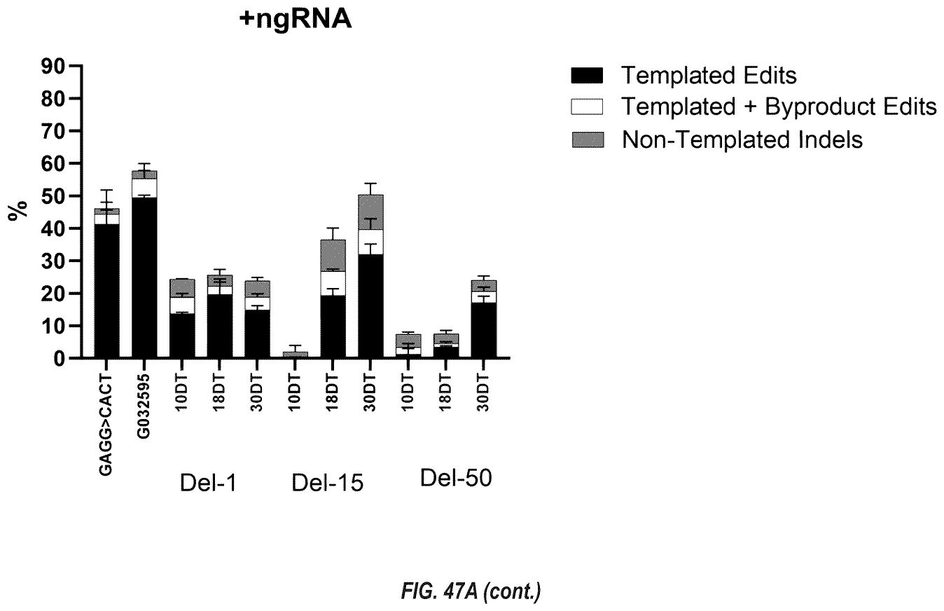

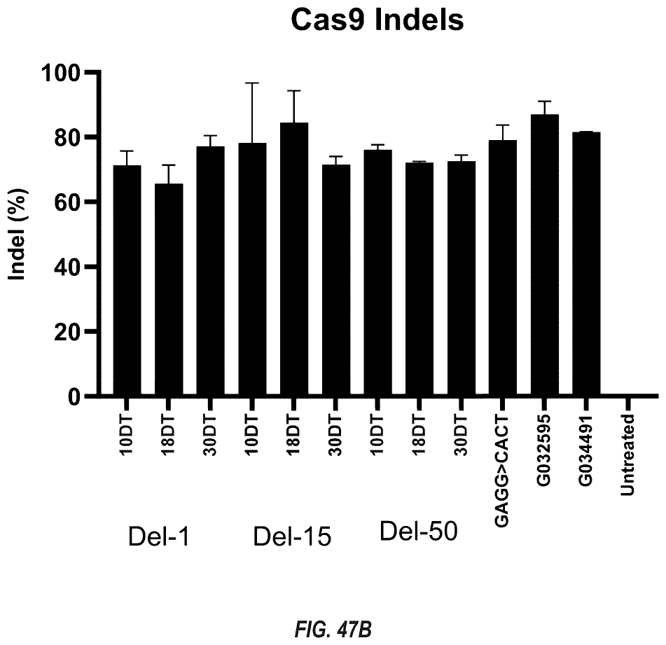

Table 52A and FIG. 46A show the insertion editing efficiency results in cells transfected with MCP-PolK-nCas9. Table 52B and FIG. 46B show the indel analysis in cells transfected with SpyCas9 and the MS2-loop-linked 3′ extensions encoding insertions. Table 53A and FIG. 47A show the deletion editing efficiency results in cells transfected with MCP-PolK-nCas9. Table 53B and FIG. 47B show the indel analysis in cells transfected with SpyCas9 and the MS2-loop-linked 3′ extensions encoding deletions.

Three measurements are reported in the analysis of insertion or deletion editing efficiency in cells transfected with MCP-PolK-nCas9: the “templated edits” corresponding to reads with the templated insertion or deletion edit as encoded in the template sequence of the corresponding MS2-loop-linked 3′ extension, the “templated+byproduct edits” corresponding to reads where the template-encoded insertion or deletion edit is observed along with byproduct edit(s) or indel(s) at or near the target site of the tgRNA, and the “non-templated indels” corresponding to reads where random insertion or deletion edit(s) not encoded by the template are observed at or near the target site of the tgRNA, as described in Example 1.

TABLE 52A
Templated insertion edits at VEGFA locus in Huh7 cells using MCP-PolK-nCas9.

| | | | | | | | | | Mean | SD of | | |
|---|---|---|---|---|---|---|---|---|---|---|---|---|
| | | | | | | | | | % | Tem- | | |
| | | | | | | | | | Tem- | plated | Mean | SD of |
| | | | | | | | Mean | SD of | plated | + | % | non- |
| | | | MS2- | | MS2-loop- | Tem- | % | Tem- | + | By- | non- | tem- |
| | | | loop- | Insertion | linked 3' | plate | Tem- | plated | By- | product | tem- | plated |
| mRNA | ngRNA | Guide | linked 3' | Length | extension | length | plated | Edits | product | Edits | plated | indels |
| ID | ID | ID | extension | (nt) | alias | (nt) | Edits | (N = 2) | Edits | (N = 2) | indels | (N = 2) |
| MCP- | N/A | G032595 | N/A | N/A | N/A | 18 | 55.75 | 3.75 | 0.90 | 0.14 | 0.45 | 0.64 |
| PolK- | | G034491 | ST036353 | N/A | GAGG > CACT | 18 | 32.45 | 9.26 | 0.50 | 0.14 | 0.35 | 0.21 |
| nCas9 | | | (SEQ | | | | | | | | | |
| (SEQ | | | ID NO: | | | | | | | | | |
| ID | | | 713) | | | | | | | | | |
| NO: | | | ST036410 | Ins-1 | 10DT | 10 | 10.25 | 1.63 | 0.20 | 0.14 | 0.15 | 0.21 |
| 1057) | | | (SEQ | | | | | | | | | |
| | | | ID NO: | | | | | | | | | |
| | | | 770) | | | | | | | | | |
| | | | ST036411 | | 18DT | 18 | 14.95 | 0.64 | 0.95 | 0.07 | 1.15 | 0.21 |
| | | | (SEQ | | | | | | | | | |
| | | | ID NO: | | | | | | | | | |
| | | | 771) | | | | | | | | | |
| | | | ST036412 | | 30DT | 30 | 6.10 | 0.85 | 0.10 | 0.00 | 0.15 | 0.07 |
| | | | (SEQ | | | | | | | | | |
| | | | ID NO: | | | | | | | | | |
| | | | 772) | | | | | | | | | |
| | | | ST036413 | Ins-3 | 18DT | 18 | 15.60 | 2.97 | 1.10 | 0.42 | 1.15 | 0.49 |
| | | | (SEQ | | | | | | | | | |
| | | | ID NO: | | | | | | | | | |
| | | | 773) | | | | | | | | | |
| | | | ST036414 | | 30DT | 30 | 4.05 | 1.48 | 0.50 | 0.71 | 0.60 | 0.71 |
| | | | (SEQ | | | | | | | | | |
| | | | ID NO: | | | | | | | | | |
| | | | 774) | | | | | | | | | |
| | | | ST036415 | | 50DT | 50 | 13.65 | 1.91 | 0.55 | 0.49 | 0.60 | 0.42 |
| | | | (SEQ | | | | | | | | | |
| | | | ID NO: | | | | | | | | | |
| | | | 774) | | | | | | | | | |
| | | | ST036416 | Ins-9 | 18DT | 18 | 7.30 | 1.56 | 0.60 | 0.42 | 0.60 | 0.42 |
| | | | (SEQ | | | | | | | | | |
| | | | ID NO: | | | | | | | | | |
| | | | 776) | | | | | | | | | |
| | | | ST036417 | | 30DT | 30 | 5.75 | 1.63 | 0.25 | 0.07 | 0.15 | 0.07 |
| | | | (SEQ | | | | | | | | | |
| | | | ID NO: | | | | | | | | | |
| | | | 777) | | | | | | | | | |
| | | | ST036418 | | 50DT | 50 | 17.10 | 2.69 | 1.30 | 0.14 | 1.70 | 0.71 |
| | | | (SEQ | | | | | | | | | |
| | | | ID NO: | | | | | | | | | |
| | | | 778) | | | | | | | | | |
| | | | ST036419 | Ins-21 | 30DT | 30 | 16.55 | 2.62 | 1.10 | 1.13 | 1.65 | 1.63 |
| | | | (SEQ | | | | | | | | | |
| | | | ID NO: | | | | | | | | | |
| | | | 779) | | | | | | | | | |
| | | | ST036420 | | 39DT | 39 | 24.25 | 0.64 | 2.55 | 0.92 | 2.70 | 0.71 |
| | | | (SEQ | | | | | | | | | |
| | | | ID NO: | | | | | | | | | |
| | | | 780) | | | | | | | | | |
| | | | ST036421 | | 50DT | 50 | 24.45 | 1.06 | 2.00 | 0.57 | 2.05 | 0.49 |
| | | | (SEQ | | | | | | | | | |
| | | | ID NO: | | | | | | | | | |
| | | | 781) | | | | | | | | | |
| | | | ST036422 | Ins-24 | 30DT | 30 | 17.25 | 6.01 | 2.65 | 0.35 | 3.20 | 0.42 |
| | | | (SEQ | | | | | | | | | |
| | | | ID NO: | | | | | | | | | |
| | | | 782) | | | | | | | | | |
| | | | ST036423 | | 50DT | 50 | 30.10 | 0.14 | 3.05 | 0.07 | 3.10 | 0.42 |
| | | | (SEQ | | | | | | | | | |
| | | | ID NO: | | | | | | | | | |
| | | | 783) | | | | | | | | | |
| | | | ST036424 | | 65DT | 65 | 27.55 | 11.24 | 3.55 | 0.35 | 3.75 | 0.07 |
| | | | (SEQ | | | | | | | | | |
| | | | ID NO: | | | | | | | | | |
| | | | 784) | | | | | | | | | |
| | | | ST036425 | Ins-39 | 46DT | 46 | 4.65 | 0.21 | 2.75 | 2.47 | 3.00 | 2.12 |
| | | | (SEQ | | | | | | | | | |
| | | | ID NO: | | | | | | | | | |
| | | | 785) | | | | | | | | | |
| | | | ST036426 | | 57DT | 57 | 11.85 | 3.18 | 2.00 | 1.41 | 2.30 | 1.13 |
| | | | (SEQ | | | | | | | | | |
| | | | ID NO: | | | | | | | | | |
| | | | 786) | | | | | | | | | |
| | | | ST036427 | | 65DT | 65 | 13.80 | 6.36 | 3.10 | 0.71 | 3.05 | 0.78 |
| | | | (SEQ | | | | | | | | | |
| | | | ID NO: | | | | | | | | | |
| | | | 787 | | | | | | | | | |
| | G034622 | G032595 | N/A | N/A | N/A | 18 | 49.45 | 0.78 | 5.85 | 2.47 | 2.40 | 2.26 |
| | | G034491 | ST036353 | N/A | GAGG > CACT | 18 | 41.25 | 10.54 | 3.00 | 1.41 | 1.85 | 1.91 |
| | | | ST036410 | Ins-1 | 10DT | 10 | 28.55 | 0.49 | 6.60 | 1.41 | 6.50 | 1.56 |
| | | | ST036411 | | 18DT | 18 | 35.30 | 3.68 | 3.15 | 3.61 | 3.60 | 4.53 |
| | | | ST036412 | | 30DT | 30 | 18.60 | 6.51 | 2.45 | 1.06 | 2.40 | 0.85 |
| | | | ST036413 | Ins-3 | 18DT | 18 | 28.90 | 0.14 | 5.05 | 2.62 | 5.00 | 2.69 |
| | | | ST036414 | | 30DT | 30 | 19.75 | 3.18 | 1.25 | 1.34 | 1.25 | 1.34 |
| | | | ST036415 | | 50DT | 50 | 30.60 | 5.80 | 2.35 | 0.21 | 2.85 | 0.49 |
| | | | ST036416 | Ins-9 | 18DT | 18 | 12.50 | 4.53 | 1.55 | 1.34 | 2.00 | 1.98 |
| | | | ST036417 | | 30DT* | 30 | 17.10 | 0.00 | 1.05 | 0.35 | 1.65 | 0.07 |
| | | | ST036418 | | 50DT | 50 | 23.40 | 1.41 | 2.40 | 0.14 | 2.30 | 0.14 |
| | | | ST036419 | Ins-21 | 30DT | 30 | 24.05 | 0.64 | 3.40 | 1.56 | 5.30 | 1.56 |
| | | | ST036420 | | 39DT | 39 | 27.65 | 8.41 | 7.05 | 2.33 | 9.75 | 3.04 |
| | | | ST036421 | | 50DT | 50 | 30.20 | 3.54 | 5.85 | 0.21 | 6.05 | 0.07 |
| | | | ST036422 | Ins-24 | 30DT | 30 | 20.75 | 2.76 | 12.45 | 6.01 | 18.25 | 6.29 |
| | | | ST036423 | | 50DT | 50 | 33.65 | 1.34 | 8.70 | 6.79 | 9.40 | 6.93 |
| | | | ST036424 | | 65DT | 65 | 32.50 | 1.27 | 6.25 | 0.21 | 6.25 | 0.21 |
| | | | ST036425 | Ins-39 | 46DT | 46 | 4.75 | 2.90 | 4.45 | 3.18 | 9.50 | 0.00 |
| | | | ST036426 | | 57DT | 57 | 12.90 | 8.49 | 7.35 | 1.63 | 9.80 | 1.84 |
| | | | ST036427 | | 65DT | 65 | 9.80 | 0.85 | 3.65 | 0.07 | 4.15 | 0.78 |
| Untreated | N/A | N/A | N/A | N/A | N/A | N/A | 0.00 | 0.00 | 0.00 | 0.00 | 0.00 | 0.00 |

*N = 2

TABLE 52B
Indel analysis at the VEGFA locus in cells transfected with SpyCas9
mRNA and MS2-loop-linked 3' extensions encoding insertions.

| | | | | | MS2-loop- | | |
|---|---|---|---|---|---|---|---|
| | | | MS2-loop- | Insertion | linked 3' | Mean | |
| mRNA | ngRNA | Guide | linked 3' | Length | extension | % | SD |
| ID | ID | ID | extension | (nt) | alias | Indels | (N = 2) |
| SpyCas9 | N/A | G034491 | ST036353 | N/A | GAGG > CACT | 79.00 | 4.67 |
| (SEQ | | | ST036410 | Ins-1 | 10DT | 74.00 | 4.95 |
| ID NO: | | | ST036411 | | 18DT | 73.15 | 4.60 |
| 1001) | | | ST036412 | | 30DT | 62.05 | 12.23 |
| | | | ST036413 | Ins-3 | 18DT | 69.45 | 3.89 |
| | | | ST036414 | | 30DT | 61.55 | 2.62 |
| | | | ST036415 | | 50DT | 62.55 | 4.88 |
| | | | ST036416 | Ins-9 | 18DT | 54.10 | 12.30 |
| | | | ST036417 | | 30DT | 52.30 | 5.52 |
| | | | ST036418 | | 50DT | 58.45 | 0.35 |
| | | | ST036419 | Ins-21 | 30DT | 61.65 | 2.47 |
| | | | ST036420 | | 39DT | 68.20 | 0.42 |
| | | | ST036421 | | 50DT | 60.15 | 2.90 |
| | | | ST036422 | Ins-24 | 30DT | 84.95 | 1.34 |
| | | | ST036423 | | 50DT | 75.05 | 3.61 |
| | | | ST036424 | | 65DT | 66.75 | 6.29 |
| | | | ST036425 | Ins-39 | 46DT | 73.35 | 1.06 |
| | | | ST036426 | | 57DT | 69.10 | 4.95 |
| | | | ST036427 | | 65DT | 69.05 | 1.77 |
| | | G032595 | N/A | N/A | N/A | 87.05 | 4.03 |
| | | G034491 | N/A | N/A | N/A | 81.55 | 0.07 |
| Untreated | | N/A | N/A | N/A | N/A | 0.00 | 0.00 |

TABLE 53A
Templated deletion edits at VEGFA locus in Huh7 cells treated with MCP-PolK-nCas9 with or without ngRNA.

| | | | | | | | | | Mean | SD of | | |
|---|---|---|---|---|---|---|---|---|---|---|---|---|
| | | | | | | | | | % | Tem | | |
| | | | | | | | | | Tem- | plated | Mean | SD of |
| | | | | | | | Mean | SD of | plated | + | % | non- |
| | | | MS2- | | MS2-loop- | Tem- | % | Tem- | + | By- | non- | tem- |
| | | | loop- | Deletion | linked 3' | plate | Tem- | plated | By- | product | tem- | plated |
| mRNA | ngRNA | Guide | linked 3' | Length | extension | length | plated | Edits | product | Edits | plated | indels |
| ID | ID | ID | extension | (nt) | alias | (nt) | Edits | (N = 2) | Edits | (N = 2) | indels | (N = 2) |
| MCP- | N/A | G032595 | N/A | N/A | N/A | 18 | 55.75 | 3.75 | 0.90 | 0.14 | 0.45 | 0.64 |
| PolK- | | G034491 | ST036353 | N/A | GAGG > CACT | 18 | 32.45 | 9.26 | 0.50 | 0.14 | 0.35 | 0.21 |
| nCas9 | | | (SEQ | | | | | | | | | |
| | | | ID NO: | | | | | | | | | |
| | | | 713) | | | | | | | | | |
| | | | ST036395 | Del-1 | 10DT | 10 | 0.60 | 0.28 | 0.00 | 0.00 | 0.25 | 0.07 |
| | | | (SEQ | | | | | | | | | |
| | | | ID NO: | | | | | | | | | |
| | | | 755) | | | | | | | | | |
| | | | ST036400 | | 18DT | 18 | 1.95 | 1.34 | 0.60 | 0.71 | 0.75 | 0.49 |
| | | | (SEQ | | | | | | | | | |
| | | | ID NO: | | | | | | | | | |
| | | | 760) | | | | | | | | | |
| | | | ST036405 | | 30DT | 30 | 3.65 | 0.07 | 1.05 | 0.21 | 1.70 | 0.14 |
| | | | (SEQ | | | | | | | | | |
| | | | ID NO: | | | | | | | | | |
| | | | 765) | | | | | | | | | |
| | | | ST036396 | Del-15 | 10DT | 10 | 0.15 | 0.07 | 0.60 | 0.42 | 1.60 | 0.99 |
| | | | (SEQ | | | | | | | | | |
| | | | ID NO: | | | | | | | | | |
| | | | 756) | | | | | | | | | |
| | | | ST036401 | | 18DT | 18 | 17.70 | 0.28 | 0.30 | 0.00 | 0.45 | 0.21 |
| | | | (SEQ | | | | | | | | | |
| | | | ID NO: | | | | | | | | | |
| | | | 761) | | | | | | | | | |
| | | | ST036406 | | 30DT | 30 | 29.30 | 6.22 | 2.20 | 0.57 | 2.75 | 0.21 |
| | | | (SEQ | | | | | | | | | |
| | | | ID NO: | | | | | | | | | |
| | | | 766) | | | | | | | | | |
| | | | ST036397 | Del-50 | 10DT | 10 | 0.05 | 0.07 | 0.45 | 0.07 | 1.10 | 0.14 |
| | | | (SEQ | | | | | | | | | |
| | | | ID NO: | | | | | | | | | |
| | | | 757) | | | | | | | | | |
| | | | ST036402 | | 18DT | 18 | 4.35 | 0.92 | 0.30 | 0.42 | 0.95 | 1.34 |
| | | | (SEQ | | | | | | | | | |
| | | | ID NO: | | | | | | | | | |
| | | | 762) | | | | | | | | | |
| | | | ST036407 | | 30DT | 30 | 16.45 | 8.84 | 0.20 | 0.00 | 0.35 | 0.21 |
| | | | (SEQ | | | | | | | | | |
| | | | ID NO: | | | | | | | | | |
| | | | 767) | | | | | | | | | |
| | G034622 | G032595 | N/A | N/A | N/A | 18 | 49.45 | 0.78 | 5.85 | 2.47 | 2.40 | 2.26 |
| | | G034491 | ST036353 | N/A | GAGG > CACT | 18 | 41.25 | 10.54 | 3.00 | 1.41 | 1.85 | 1.91 |
| | | | ST036395 | Del-1 | 10DT | 10 | 13.65 | 0.49 | 5.10 | 1.13 | 5.55 | 0.21 |
| | | | ST036400 | | 18DT | 18 | 19.65 | 4.74 | 2.50 | 1.27 | 3.45 | 1.77 |
| | | | ST036405 | | 30DT | 30 | 14.85 | 1.34 | 4.00 | 0.99 | 4.95 | 1.06 |
| | | | ST036396 | Del-15 | 10DT | 10 | 0.00 | 0.00 | 0.25 | 0.07 | 1.70 | 1.98 |
| | | | ST036401 | | 18DT | 18 | 19.35 | 2.05 | 7.45 | 0.64 | 9.65 | 3.61 |
| | | | ST036406 | | 30DT | 30 | 31.95 | 3.18 | 7.65 | 3.32 | 10.75 | 3.46 |
| | | | ST036397 | Del-50 | 10DT | 10 | 1.30 | 1.70 | 2.00 | 1.27 | 4.15 | 0.64 |
| | | | ST036402 | | 18DT | 18 | 3.50 | 0.28 | 1.05 | 0.49 | 2.95 | 1.06 |
| | | | ST036407 | | 30DT | 30 | 17.10 | 1.98 | 3.45 | 1.34 | 3.45 | 1.34 |
| Untreated | N/A | N/A | N/A | N/A | N/A | N/A | 0.00 | 0.00 | 0.00 | 0.00 | 0.00 | 0.00 |

TABLE 53B
Indel analysis at the VEGFA locus in cells transfected with SpyCas9
mRNA and MS2-loop-linked 3' extensions encoding deletions.

| | | | | | MS2-loop- | | |
|---|---|---|---|---|---|---|---|
| | | | MS2-loop- | Deletion | linked 3' | Mean | |
| mRNA | ngRNA | Guide | linked 3' | Length | extension | % | SD |
| ID | ID | ID | extension | (nt) | alias | Indels | (N = 2) |
| SpyCas9 | N/A | G034491 | ST036353 | N/A | GAGG > CACT | 79.00 | 4.67 |
| | | G034491 | ST036395 | Del-1 | 10DT | 71.30 | 4.38 |
| | | | ST036400 | | 18DT | 65.70 | 5.66 |
| | | | ST036405 | | 30DT | 77.20 | 3.25 |
| | | | ST036396 | Del-15 | 10DT | 78.20 | 18.53 |
| | | | ST036401 | | 18DT | 84.50 | 9.76 |
| | | | ST036406 | | 30DT | 71.45 | 2.62 |
| | | | ST036397 | Del-50 | 10DT | 76.10 | 1.56 |
| | | | ST036402 | | 18DT | 72.15 | 0.35 |
| | | | ST036407 | | 30DT | 72.60 | 1.84 |
| | | G032595 | N/A | N/A | N/A | 87.05 | 4.03 |
| | | G034491 | N/A | N/A | N/A | 81.55 | 0.07 |
| Untreated | N/A | N/A | N/A | N/A | N/A | 0.00 | 0.00 |

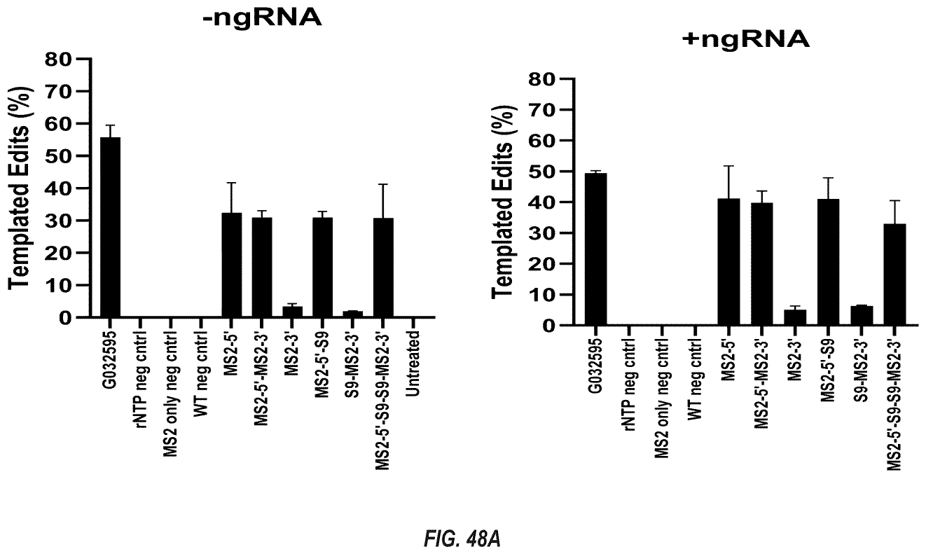

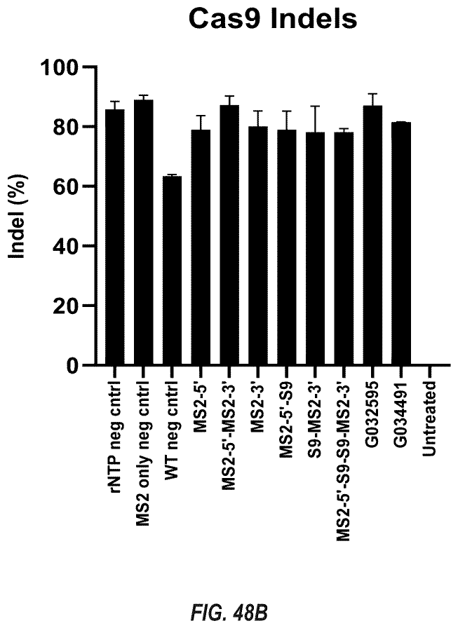

To assess the effect on editing efficiency of various numbers and positions of MS2-loops within the MS2-loop-linked 3′ extension, MS2-loop-linked 3′ extensions were generated with an MS2 loop at the 3′-end, 5′-end, or both ends, with or without linkers. Huh7 cells were prepared as described in Example 1, and plated at a density of 15,000 cells/well. After 24 hours, of incubation at 37° C., 5% CO₂, cells were transfected using Lipofectamine™ MessengerMAX™ Reagent (Invitrogen, Cat. LMRNA015) according to manufacturer's instructions with 300 ng of mRNA, sgRNA (G034491) at a final concentration of 25 nM, and either a MS2-loop-linked 3′ extension at a final concentration of 25 nM, or a MS2-loop-linked 3′ extension and a ngRNA at a 3:2 molar ratio respectively (15 nM:10 nM). Cells treated with only tgRNA (G032595) or sgRNA (G034491) (i.e., no MS2-loop-linked 3′ extension) were transfected with 300 ng of mRNA and a final concentration of 25 nM of gRNA. Samples were prepared in duplicate. After transfection and incubation at 37° C., 5% CO₂ for 72 hours, samples were prepared for next-generation sequencing (NGS) and analysis for editing efficiency as described in Example 1. Table 54A and FIG. 48A show the templated editing data at the VEGFA locus. Table 54B and FIG. 48B show the indel analysis using SpyCas9.

TABLE 54A
Templated editing analysis in Huh7 cells treated with MS2-loop-linked 3' extensions differing in
the number and position of MS2 loops and internal linkers; without or with nicking guides.

| | | | | | | | | SD of | | |
|---|---|---|---|---|---|---|---|---|---|---|
| | | | | | | | Mean % | Templated | | |
| | | | | | | | Templated | + | | |
| | | | | | | SD of | + | By- | | |
| | | | MS2-loop- | | Mean % | Templated | By- | product | Mean | SD of |
| mRNA | ngRNA | Guide | linked 3' | MS2-loop-linked 3' | Templated | Edits | product | Edits | % | Indels |
| ID | ID | ID | extension | extension alias | Edits | (N = 2) | Edits | (N = 2) | Indels | (N=2) |
| MCP- | N/A | G032595 | N/A | N/A | 55.75 | 3.75 | 0.90 | 0.14 | 0.45 | 0.64 |
| PolK- | | G034491 | ST036350 | rNTP neg cntrl | 0.00 | 0.00 | 0.00 | 0.00 | 0.35 | 0.07 |
| nCas9 | | | (SEQ ID | | | | | | | |
| | | | NO: 710) | | | | | | | |
| | | | ST036351 | MS2 only neg cntrl | 0.00 | 0.00 | 0.00 | 0.00 | 1.85 | 2.33 |
| | | | (SEQ ID | | | | | | | |
| | | | NO: 711) | | | | | | | |
| | | | ST036352 | WT neg cntrl | 0.00 | 0.00 | 0.00 | 0.00 | 0.50 | 0.14 |
| | | | (SEQ ID | | | | | | | |
| | | | NO: 712) | | | | | | | |
| | | | ST036353 | MS2-5' | 32.45 | 9.26 | 0.50 | 0.14 | 0.35 | 0.21 |
| | | | (SEQ ID | | | | | | | |
| | | | NO: 713) | | | | | | | |
| | | | ST036354 | MS2-5'-MS2-3' | 30.95 | 2.05 | 1.15 | 1.06 | 0.00 | 0.00 |
| | | | (SEQ ID | | | | | | | |
| | | | NO: 714) | | | | | | | |
| | | | ST036355 | MS2-3' | 3.35 | 0.92 | 0.10 | 0.00 | 1.70 | 0.85 |
| | | | (SEQ ID | | | | | | | |
| | | | NO: 715) | | | | | | | |
| | | | ST036356 | MS2-5'-S9 | 30.95 | 1.91 | 1.05 | 0.35 | 0.65 | 0.21 |
| | | | (SEQ ID | | | | | | | |
| | | | NO: 716) | | | | | | | |
| | | | ST036357 | S9-MS2-3' | 1.95 | 0.07 | 0.45 | 0.49 | 1.35 | 0.49 |
| | | | (SEQ ID | | | | | | | |
| | | | NO: 717) | | | | | | | |
| | | | ST036358 | MS2-5'-S9-S9-MS2-3' | 30.80 | 10.47 | 0.65 | 0.07 | 0.80 | 0.14 |
| | | | (SEQ ID | | | | | | | |
| | | | NO: 718) | | | | | | | |
| | G034622 | G032595 | N/A | N/A | 49.45 | 0.78 | 5.85 | 2.47 | 2.40 | 2.26 |
| | | G034491 | ST036350 | rNTP neg cntrl | 0.00 | 0.00 | 0.00 | 0.00 | 0.45 | 0.35 |
| | | | ST036351 | MS2 only neg cntrl | 0.00 | 0.00 | 0.30 | 0.28 | 7.6 | 0.00 |
| | | | ST036352 | WT neg cntrl | 0.00 | 0.00 | 0.05 | 0.07 | 2.85 | 0.64 |
| | | | ST036353 | MS2-5' | 41.25 | 10.54 | 3.00 | 1.41 | 1.85 | 1.91 |
| | | | ST036354 | MS2-5'-MS2-3' | 39.80 | 3.82 | 4.60 | 2.69 | 2.65 | 1.91 |
| | | | ST036355 | MS2-3' | 5.00 | 1.27 | 0.85 | 1.06 | 4.25 | 4.45 |
| | | | ST036356 | MS2-5'-S9 | 41.05 | 6.86 | 2.90 | 0.85 | 1.75 | 1.06 |
| | | | ST036357 | S9-MS2-3' | 6.30 | 0.28 | 0.50 | 0.57 | 4.80 | 0.85 |
| | | | ST036358 | MS2-5'-S9-S9-MS2-3' | 33.05 | 7.42 | 3.10 | 0.57 | 2.05 | 0.92 |
| Untreated | N/A | N/A | N/A | N/A | 0.00 | 0.00 | 0.00 | 0.00 | 0.00 | 0.00 |

TABLE 54B
Indel analysis using SpyCas9.

| mRNA | ngRNA | Guide | MS2-loop-linked | MS2-loop-linked 3' | Mean % | SD |
|---|---|---|---|---|---|---|
| ID | ID | ID | 3' extension | extension alias | Indels | (N=2) |
| SpyCas9 | N/A | G034491 | ST036350 | rNTP neg cntrl | 85.85 | 2.62 |
| | | | ST036351 | MS2 only neg cntrl | 89.05 | 1.48 |
| | | | ST036352 | WT neg cntrl | 63.40 | 0.57 |
| | | | ST036353 | MS2-5' | 79.00 | 4.67 |
| | | | ST036354 | MS2-5'-MS2-3' | 87.20 | 3.11 |
| | | | ST036355 | MS2-3' | 80.15 | 5.16 |
| | | | ST036356 | MS2-5'-S9 | 79.05 | 6.15 |
| | | | ST036357 | S9-MS2-3' | 78.10 | 8.77 |
| | | | ST036358 | MS2-5'-S9-S9-MS2-3' | 78.15 | 1.20 |
| | | G032595 | N/A | tgRNA | 87.05 | 4.03 |
| | | G034491 | N/A | sgRNA | 81.55 | 0.07 |
| Untreated | N/A | N/A | N/A | N/A | 0.00 | 0.00 |

### Example 22—Templated Editing with DRS Containing Mismatches Relative to the Genomic Locus with DNA Polymerase K

This Example assessed template-based editing with a DRS not fully complementary to the genomic sequence. Mismatches between the DRS and the genomic sequence also resulted in mismatches between the guide spacer sequence and the DRS. Indel analysis and editing analysis were performed using guide RNAs (gRNAs) targeting the VEGFA locus in Huh7 cells. These gRNAs included the same sgRNA (G034491) paired with an aptamer in the 3′ extension region of the tgRNA (MS2-loop-linked 3′ extension) comprising a template sequence and a DRS sequence including one mismatch relative to the genomic sequence.

To assess editing efficiency with one mismatch between the 11-nucleotide DRS and the genomic sequence, 11 MS2-loop-linked 3′ extensions were generated each with one mismatch at a different nucleotide position within the DRS sequence. The position numbering for the mismatch was counted from the first nucleotide of the DRS onward in the 5′ to 3′ direction from the DRS/template junction. The DRS included 2′OMe modifications at positions 1-3 (D1-D3) and 6-11 (D6-D11) from the DRS/template junction in the 5′ to 3′ direction.

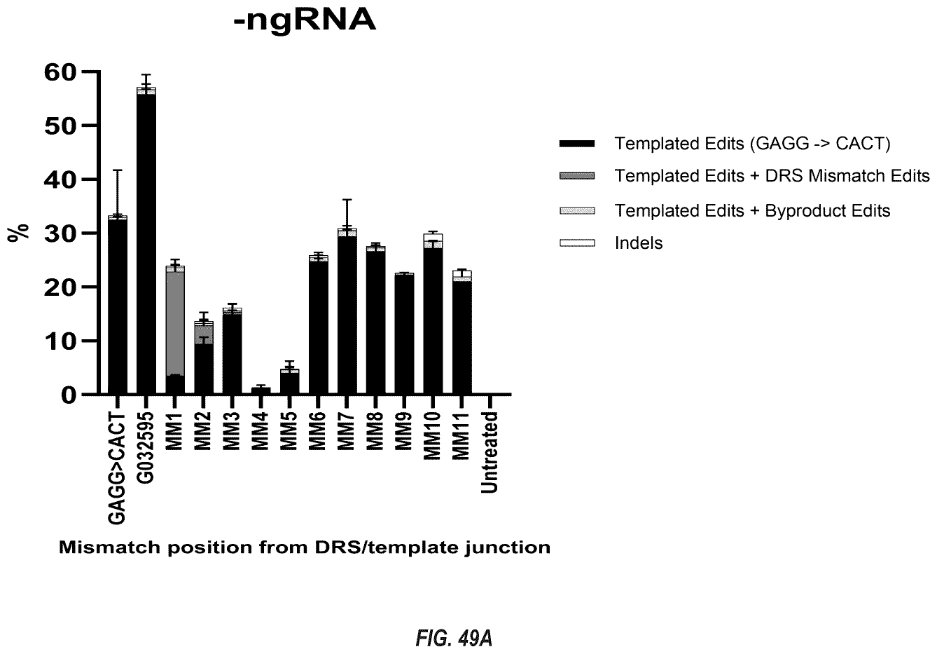

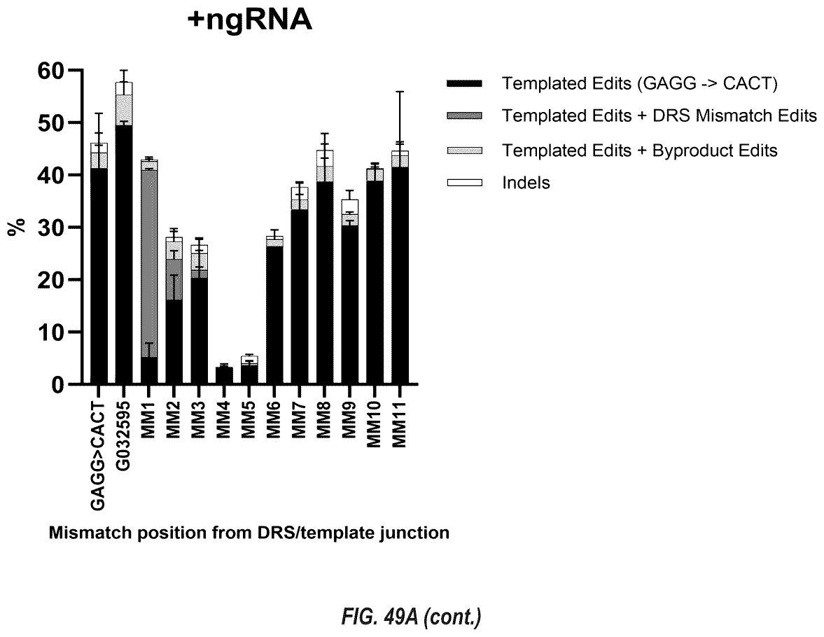

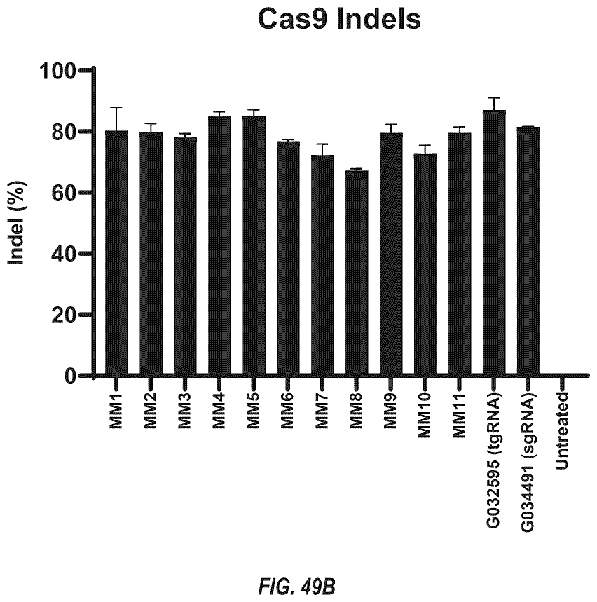

Huh7 cells were prepared as described in Example 1, and plated at a density of 15,000 cells/well. After 24 hours, of incubation at 37° C., 5% CO₂, cells were transfected using Lipofectamine™ MessengerMAX™ Reagent (Invitrogen, Cat. LMRNA015) according to manufacturer's instructions with 300 ng of mRNA (encoding SpyCas9 or MCP-PolK-nCas9), sgRNA (G034491) at a final concentration of 25 nM, and either a MS2-loop-linked 3′ extension at a final concentration of 25 nM, or a MS2-loop-linked 3′ extension and a ngRNA at a 3:2 molar ratio respectively (15 nM:10 nM). Cells treated with only tgRNA (G032595) or sgRNA (G034491) (i.e., no MS2-loop-linked 3′ extension) were transfected with 300 ng of mRNA and a final concentration of 25 nM of gRNA. Samples were prepared in duplicate. After transfection and incubation at 37° C., 5% CO₂ for 72 hours, samples were prepared for next-generation sequencing (NGS) and analysis for editing efficiency as described in Example 1. Table 55A and FIG. 49A show the editing data at the VEGFA locus in cells treated with MCP-PolK-nCas9. In FIG. 49A, four measurements are shown: the “templated edits” corresponding to reads with the GAGG>CACT edit as encoded in the template sequence of the corresponding MS2-loop-linked 3′ extension, the “templated edits+DRS mismatch edits” corresponding to reads where both the template-encoded GAGG>CACT edit and the edit complementary to the mismatch in the DRS are observed, the “templated edits+byproduct edits” corresponding to reads where the template-encoded GAGG>CACT edit encoded in the template is observed along with byproduct edit(s) at or near the target site of the tgRNA, and the “indels” corresponding to reads where indels are observed at or near the target site of the tgRNA, as described in Example 1. Table 55B and FIG. 49B show the indel data at the VEGFA locus in cells treated with SpyCas9.

TABLE 55A
Editing analysis at the VEGFA locus in Huh7 cells treated with
MS2-loop-linked 3′ extensions encoding the GAGG >
CACT edit with an additional mismatch in the DRS at varying positions.

| | | | | MS2-loop- | | SD of |
|---|---|---|---|---|---|---|
| | | | MS2-loop- | linked 3′ | Mean % | templated |
| mRNA | ngRNA | Guide | linked 3′ | extension | templated | edits |
| ID | ID | ID | extension | alias | edits | (N = 2) |
| MCP- | N/A | G032595 | N/A | N/A | 55.75 | 3.75 |
| PolK- | | G034491 | ST036353 | GAGG > CACT | 32.45 | 9.26 |
| nCas9 | | | (SEQ ID | | | |
| | | | NO: 713) | | | |
| | | | ST036438 | MM1 | 3.45 | 0.2 |
| | | | (SEQ ID | | | |
| | | | NO: 798) | | | |
| | | | ST036437 | MM2 | 9.35 | 1.34 |
| | | | (SEQ ID | | | |
| | | | NO: 797) | | | |
| | | | ST036436 | MM3 | 14.85 | 2.05 |
| | | | (SEQ ID | | | |
| | | | NO: 796) | | | |
| | | | ST036435 | MM4 | 1.20 | 0.57 |
| | | | (SEQ ID | | | |
| | | | NO: 795) | | | |
| | | | ST036434 | MM5 | 4.05 | 2.19 |
| | | | (SEQ ID | | | |
| | | | NO: 794) | | | |
| | | | ST036433 | MM6 | 24.70 | 0.57 |
| | | | (SEQ ID | | | |
| | | | NO: 793) | | | |
| | | | ST036432 | MM7 | 29.35 | 6.86 |
| | | | (SEQ ID | | | |
| | | | NO: 792) | | | |
| | | | ST036431 | MM8 | 26.60 | 1.56 |
| | | | (SEQ ID | | | |
| | | | NO: 791) | | | |
| | | | ST036430 | MM9 | 22.25 | 0.35 |
| | | | (SEQ ID | | | |
| | | | NO: 790) | | | |
| | | | ST036429 | MM10 | 27.20 | 1.27 |
| | | | (SEQ ID | | | |
| | | | NO: 789) | | | |
| | | | ST036428 | MM11* | 21.00* | N/A |
| | | | (SEQ ID | | | |
| | | | NO: 788) | | | |
| | G034622 | G032595 | N/A | N/A | 49.45 | 0.78 |
| | | G034491 | ST036353 | GAGG > CACT | 41.25 | 10.54 |
| | | | ST036438 | MM1 | 5.20 | 2.69 |
| | | | ST036437 | MM2 | 16.15 | 4.74 |
| | | | ST036436 | MM3 | 20.25 | 5.30 |
| | | | ST036435 | MM4 | 3.10 | 0.85 |
| | | | ST036434 | MM5 | 3.60 | 0.85 |
| | | | ST036433 | MM6* | 26.40* | N/A |
| | | | ST036432 | MM7 | 33.40 | 5.09 |
| | | | ST036431 | MM8 | 38.65 | 9.26 |
| | | | ST036430 | MM9 | 30.30 | 0.99 |
| | | | ST036429 | MM10 | 38.90 | 3.39 |
| | | | ST036428 | MM11 | 41.45 | 14.50 |
| Untreated | N/A | N/A | N/A | N/A | 0.00 | 0.00 |

| | | SD of | | | | |
|---|---|---|---|---|---|---|
| | Mean % | templated | | SD of | | |
| | templated | edits + DRS | Mean % | Templated + | | |
| | edits + DRS | mismatch | Templated + | Byproduct | | SD of |
| mRNA | mismatch | edits | Byproduct | Edits | Mean % | Indels |
| ID | edits | (N = 2) | Edits | (N = 2) | Indels | (N = 2) |
| MCP- | N/A | N/A | 0.90 | 0.14 | 0.45 | 0.64 |
| PolK- | N/A | N/A | 0.50 | 0.14 | 0.35 | 0.21 |
| nCas9 | 19.35 | 2.33 | 0.80 | 0.42 | 0.30 | 0.28 |
| | 3.45 | 2.47 | 0.45 | 0.49 | 0.35 | 0.35 |
| | 0.45 | 0.35 | 0.25 | 0.07 | 0.65 | 0.64 |
| | 0.00 | 0.00 | 0.00 | 0.00 | 0.10 | 0.00 |
| | 0.00 | 0.00 | 0.60 | 0.57 | 0.15 | 0.21 |
| | 0.00 | 0.00 | 0.75 | 0.49 | 0.40 | 0.57 |
| | 0.00 | 0.00 | 1.10 | 0.28 | 0.45 | 0.49 |
| | 0.00 | 0.00 | 0.70 | 0.28 | 0.25 | 0.21 |
| | 0.00 | 0.00 | 0.30 | 0.00 | 0.05 | 0.07 |
| | 0.00 | 0.00 | 1.30 | 0.14 | 1.35 | 0.49 |
| | 0.00* | N/A | 0.95 | 1.34 | 1.10* | N/A |
| | N/A | N/A | 5.85 | 2.47 | 2.40 | 2.26 |
| | N/A | N/A | 3.00 | 1.41 | 1.85 | 1.91 |
| | 35.70 | 0.28 | 1.70 | 0.85 | 0.30 | 0.14 |
| | 7.80 | 1.56 | 3.35 | 2.47 | 0.85 | 1.06 |
| | 1.55 | 0.64 | 3.20 | 2.97 | 1.60 | 1.13 |
| | 0.00 | 0.00 | 0.05 | 0.07 | 0.15 | 0.21 |
| | 0.00 | 0.00 | 0.45 | 0.49 | 1.40 | 0.28 |
| | 0.00* | 0.00 | 1.30 | 1.84 | 0.60* | N/A |
| | 0.00 | 0.00 | 1.90 | 0.99 | 2.40 | 0.99 |
| | 0.00 | 0.00 | 3.00 | 1.56 | 3.05 | 1.20 |
| | 0.00 | 0.00 | 2.25 | 0.35 | 2.80 | 1.70 |
| | 0.00 | 0.00 | 2.25 | 0.92 | 0.15 | 0.21 |
| | 0.00 | 0.00 | 2.25 | 2.62 | 0.90 | 1.27 |
| Untreated | N/A | N/A | 0.00 | 0.00 | 0.00 | 0.00 |

*N = 1

TABLE 55B
Indel analysis using SpyCas9.

| mRNA | ngRNA | Guide | MS2-loop-linked | MS2-loop-linked | Mean % | SD |
|---|---|---|---|---|---|---|
| ID | ID | ID | 3′ extension | 3′ extension alias | Indels | (N = 2) |
| SpyCas9 | N/A | G034491 | ST036353 | GAGG > CACT | 79.00 | 4.67 |
| | | | ST036438 | MM1 | 80.25 | 7.71 |
| | | | ST036437 | MM2 | 79.90 | 2.69 |
| | | | ST036436 | MM3 | 78.00 | 1.27 |
| | | | ST036435 | MM4 | 85.20 | 1.27 |
| | | | ST036434 | MM5 | 85.00 | 2.12 |
| | | | ST036433 | MM6 | 76.80 | 0.57 |
| | | | ST036432 | MM7 | 72.30 | 3.54 |
| | | | ST036431 | MM8 | 67.20 | 0.57 |
| | | | ST036430 | MM9 | 79.50 | 2.83 |
| | | | ST036429 | MM10 | 72.60 | 2.83 |
| | | | ST036428 | MM11 | 79.50 | 1.98 |
| | | G032595 | N/A | tgRNA | 87.05 | 4.03 |
| | | G034491 | N/A | sgRNA | 81.55 | 0.07 |
| Untreated | | N/A | N/A | Untreated | 0.00 | 0.00 |

### Example 23—Dose Response of Templated Editing in Primary Human Hepatocytes with tgRNAs Comprising Various Chemical Modifications in the Presence of Enhancers (Vpx and FEN1) of Template-Based Editing with Polymerase K and Phi29, Including Variants

This Example evaluated templated editing efficiency with tgRNAs having various chemical modifications in the 3′ end of the tgRNA. In particular, the following chemical modifications were assessed: phosphorothioate (PS) bonds on the 3′-end, 2′-O-methyl-U on the 3′-end, or 2′-O-methyl modified (2′-Ome) nucleotides. See Table 56 for further description of the tgRNAs and the position of the chemical modifications.

Templated guide RNAs (tgRNAs) targeting the VEGFA locus with varying amounts of phosphorothioate (PS) bonds on the 3′-end, 2′-O-methyl-U on the 3′-end, or 2′-O-methyl modified (2′-Ome) nucleotides within the DRS were delivered to primary human hepatocytes (PHH) to assess SpyCas9 indel activity and template-based editing efficiency. Editing was also assessed in the presence of enhancers Vpx and FEN1.

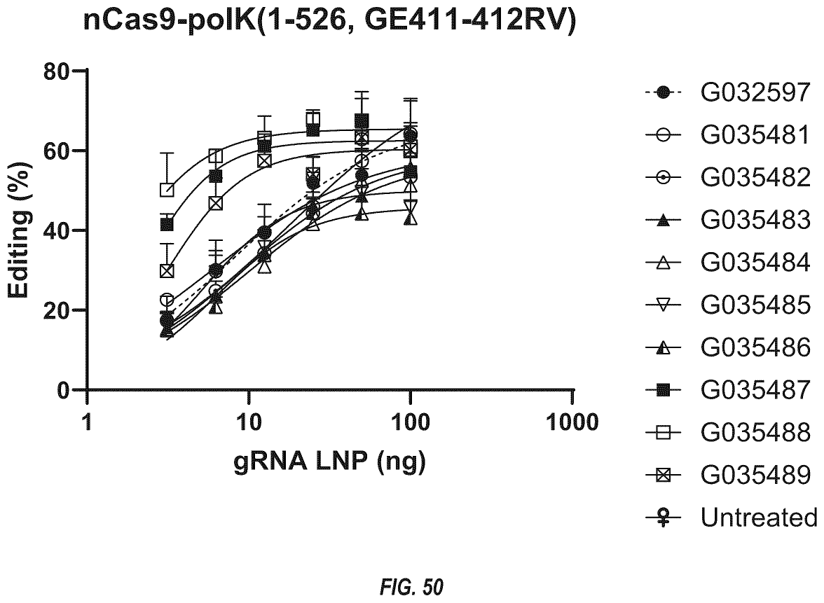

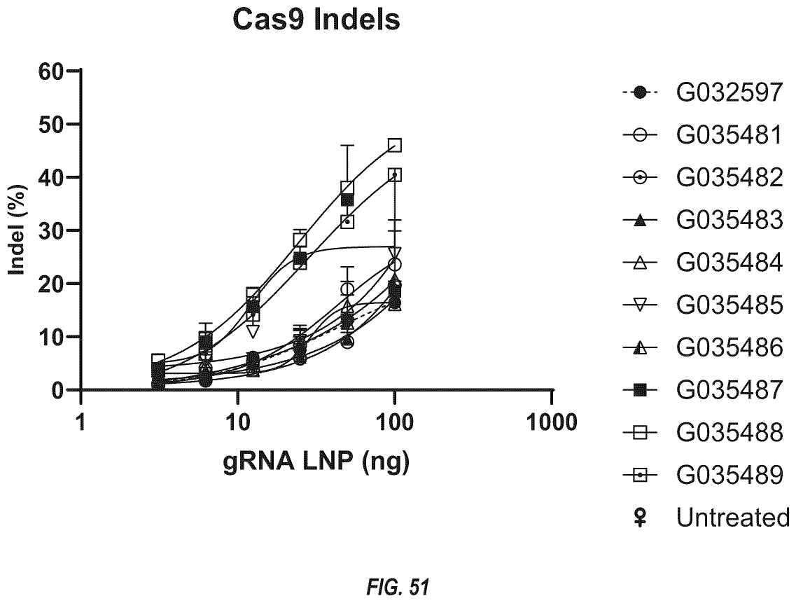

Primary human hepatocytes (Gibco, Lot 8290) were prepared as described in Example 1, and plated at a density of 30,000 cells/well. Plated cells were allowed to settle and adhere for 4 hours in a tissue culture incubator at 37° C. and 5% CO₂ atmosphere. After incubation, cells were checked for monolayer formation and washed once with hepatocyte maintenance medium (Gibco Williams' Medium E, Cat. A12176-01) plus cell maintenance supplements (Gibco, Cat. A15564 CM4000). After 24 hours from plating, LNPs comprising a single RNA species were used for lipofection. The LNPs were prepared as described in Example 1. The LNPs were made with a molar ratio of 50% Lipid A, 38% cholesterol, 9% DSPC, and 3% PEG2k-DMG, and formulated with a lipid amine to RNA phosphate (N:P) molar ratio of about 6. LNPs were incubated in media containing 6% fetal bovine serum (FBS) at 37° C. for 10 minutes, and were added to the PHH as follows. A LNP comprising a tgRNA, a LNP comprising a Vpx-encoding mRNA, and a LNP comprising either a SpyCas9-encoding mRNA (SEQ ID NO: 1001) or a nCas9-polK(1-526, GE411-412RV) encoding mRNA (SEQ ID NO: 1232) were delivered to the cells. The tgRNAs were titrated in a 7-point, 2-fold dose response curve with a top dose of 100 ng. The mRNA cargo was kept constant with the SpyCas9 encoding mRNA or the nCas9-polK(1-526, GE411-412RV) encoding mRNA at 200 ng/reaction, and the Vpx-encoding mRNA at 10 ng/reaction. After delivery and incubation at 37° C., 5% CO₂ for 72 hours, samples were prepared for next-generation sequencing (NGS) and analysis for editing efficiency as described in Example 1. Table 56 and FIG. 50 show templated editing efficiency results. Table 57 and FIG. 51 show SpyCas9 indel efficiency. The “untreated” controls did not receive any mRNA or gRNA.

TABLE 56
Templated editing efficiency with chemically modified
tgRNAs at the VEGFA locus in primary human hepatocytes
treated with nCas9-polK(1-526, GE411-412RV) and Vpx.

| | Chemical | gRNA | Mean | SD | |
|---|---|---|---|---|---|
| | modification in | Concentra- | % | (N = | EC50 |
| Guide ID | guide | tion (ng) | Editing | 2) | (ng) |
| G032597 | one PS bond on the | 100 | 63.65 | 0.50 | 8.475 |
| (SEQ ID | 3′-end | 50 | 53.85 | 6.72 | |
| NO: 530) | | 25 | 51.85 | 6.72 | |
| | | 12.5 | 39.45 | 7.14 | |
| | | 6.25 | 30.20 | 3.54 | |
| | | 3.125 | 17.70 | 5.80 | |
| | | 0 | 0.35 | 0.50 | |
| G035481 | two PS bonds on | 100 | 53.45 | 4.88 | 6.079 |
| (SEQ ID | the 3′-end | 50 | 57.45 | 11.53 | |
| NO: 531) | | 25 | 46.05 | 2.33 | |
| | | 12.5 | 39.50 | 3.96 | |
| | | 6.25 | 29.80 | 7.78 | |
| | | 3.125 | 22.65 | 0.50 | |
| | | 0 | 0.00 | 0.00 | |
| G035482 | three PS bonds on | 100 | 64.20 | 2.83 | 18.06 |
| (SEQ ID | the 3′-end | 50 | 62.90 | 2.26 | |
| NO: 532) | | 25 | 44.30 | 0.85 | |
| | | 12.5 | 34.30 | 5.80 | |
| | | 6.25 | 24.85 | 2.47 | |
| | | 3.125 | 17.35 | 0.64 | |
| | | 0 | 0.00 | 0.00 | |
| G035483 | eight 2′-Ome | 100 | 56.00 | 7.50 | 8.919 |
| (SEQ ID | nucleotide in the | 50 | 48.65 | 17.89 | |
| NO: 533) | DRS | 25 | 46.30 | 6.51 | |
| | | 12.5 | 34.65 | 0.50 | |
| | | 6.25 | 23.80 | 1.56 | |
| | | 3.125 | 15.65 | 3.75 | |
| | | 0 | 0.00 | 0.00 | |
| G035484 | nine 2′-Ome | 100 | 51.30 | 14.85 | 9.946 |
| (SEQ ID | nucleotides in the | 50 | 52.40 | 7.78 | |
| NO: 534) | DRS | 25 | 41.65 | 6.29 | |
| | | 12.5 | 30.95 | 0.21 | |
| | | 6.25 | 23.45 | 0.07 | |
| | | 3.125 | 15.25 | 4.46 | |
| | | 0 | 0.00 | 0.00 | |
| G035485 | ten 2′-Ome | 100 | 45.70 | 8.49 | 5.161 |
| (SEQ ID | nucleotides in the | 50 | 49.70 | 10.04 | |
| NO: 535) | DRS | 25 | 53.05 | 0.50 | |
| | | 12.5 | 35.85 | 4.31 | |
| | | 6.25 | 29.55 | 5.45 | |
| | | 3.125 | 16.20 | 2.97 | |
| | | 0 | 0.00 | 0.00 | |
| G035486 | eleven 2′-Ome | 100 | 43.10 | 4.53 | 6.3 |
| (SEQ ID | nucleotides in the | 50 | 44.20 | 11.31 | |
| NO: 536) | DRS | 25 | 44.45 | 5.16 | |
| | | 12.5 | 33.85 | 0.64 | |
| | | 6.25 | 20.85 | 2.47 | |
| | | 3.125 | 14.95 | 4.31 | |
| | | 0 | 0.00 | 0.00 | |
| G035487 | one 2′-O-methyl-U | 100 | 54.70 | 17.82 | 2.243 |
| (SEQ ID | on the 3′-end | 50 | 67.25 | 5.87 | |
| NO: 537 | | 25 | 65.25 | 4.31 | |
| | | 12.5 | 61.15 | 3.04 | |
| | | 6.25 | 53.65 | 6.72 | |
| | | 3.125 | 41.50 | 2.69 | |
| | | 0 | 0.00 | 0.00 | |
| G035488 | two 2′-O-methyl- | 100 | 61.00 | 3.25 | 1.598 |
| (SEQ ID | Us on the 3′-end | 50 | 67.60 | 7.21 | |
| NO: 538) | | 25 | 67.75 | 2.47 | |
| | | 12.5 | 63.25 | 5.44 | |
| | | 6.25 | 58.70 | 0.00 | |
| | | 3.125 | 50.15 | 9.26 | |
| | | 0 | 1.60 | 2.26 | |
| G035489 | three 2′-O-methyl- | 100 | 60.20 | 12.87 | 3.148 |
| (SEQ ID | Us on the 3′-end | 50 | 63.35 | 1.63 | |
| NO: 539) | | 25 | 54.10 | 4.24 | |
| | | 12.5 | 57.50 | 2.40 | |
| | | 6.25 | 46.85 | 6.29 | |
| | | 3.125 | 29.85 | 6.86 | |
| | | 0 | 0.00 | 0.00 | |
| Untreated | N/A | 0 | 0.00 | 0.00 | N/A |

TABLE 57
Indel percentage with chemically modified tgRNAs at the VEGFA locus
in primary human hepatocytes treated with SpyCas9 and Vpx.

| | gRNA Concentration | | SD | EC50 |
|---|---|---|---|---|
| Guide ID | (ng) | Mean % Indels | (N = 2) | (ng) |
| G032597 | 100 | 16.45 | 4.03 | 42.79 |
| | 50 | 13.05 | 0.92 | |
| | 25 | 7.50 | 3.68 | |
| | 12.5 | 6.20 | 0.57 | |
| | 6.25 | 1.70 | 1.84 | |
| | 3.125 | 1.80 | 0.99 | |
| | 0 | 0.00 | 0.00 | |
| G035481 | 100 | 23.65 | 6.29 | 43.25 |
| | 50 | 18.95 | 1.48 | |
| | 25 | 8.45 | 3.75 | |
| | 12.5 | 5.45 | 0.49 | |
| | 6.25 | 4.10 | 0.42 | |
| | 3.125 | 0.85 | 0.64 | |
| | 0 | 0.00 | 0.00 | |
| G035482 | 100* | 19.70* | N/A | Unstable |
| | 50 | 9.00 | 1.84 | |
| | 25 | 5.90 | 4.1 | |
| | 12.5 | 3.75 | 1.06 | |
| | 6.25 | 1.75 | 2.48 | |
| | 3.125 | 1.20 | 0.42 | |
| | 0 | 0.00 | 0.00 | |
| G035483 | 100 | 17.35 | 0.35 | Unstable |
| | 50 | 9.60 | 3.25 | |
| | 25 | 6.15 | 5.16 | |
| | 12.5 | 5.00 | 1.98 | |
| | 6.25 | 3.10 | 0.57 | |
| | 3.125 | 1.10 | 0.57 | |
| G035484 | 100* | 16.20* | N/A | 28.96 |
| | 50 | 15.80 | 7.35 | |
| | 25 | 7.40 | 1.27 | |
| | 12.5 | 3.80 | 0.71 | |
| | 6.25 | 4.55 | 2.62 | |
| | 3.125 | 1.30 | 1.41 | |
| G035485 | 100 | 25.55 | 6.44 | Unstable |
| | 50 | 12.45 | 5.45 | |
| | 25 | 9.20 | 0.85 | |
| | 12.5 | 10.95 | 6.58 | |
| | 6.25 | 5.40 | 2.26 | |
| | 3.125 | 1.90 | 0.57 | |
| G035486 | 100 | 20.75 | 0.92 | Unstable |
| | 50 | 12.70 | 1.56 | |
| | 25 | 8.55 | 3.04 | |
| | 12.5 | 5.85 | 0.64 | |
| | 6.25 | 3.40 | 2.26 | |
| | 3.125 | 1.05 | 1.20 | |
| G035487 | 100 | 18.55 | 23.12 | 12.10 |
| | 50 | 35.8 | 1.13 | |
| | 25 | 24.75 | 2.19 | |
| | 12.5 | 15.70 | 0.99 | |
| | 6.25 | 9.00 | 1.98 | |
| | 3.125 | 4.00 | 0.00 | |
| G035488 | 100 | 46.00 | 1.27 | 24.53 |
| | 50 | 38.05 | 7.99 | |
| | 25 | 28.20 | 1.98 | |
| | 12.5 | 17.95 | 1.34 | |
| | 6.25 | 9.60 | 2.97 | |
| | 3.125 | 5.45 | 1.34 | |
| G035489 | 100 | 40.45 | 1.20 | 28.56 |
| | 50 | 31.65 | 5.16 | |
| | 25 | 23.95 | 0.35 | |
| | 12.5 | 14.15 | 1.63 | |
| | 6.25 | 6.85 | 3.04 | |
| | 3.125 | 3.65 | 0.78 | |
| Untreated | 0 | 0.00 | 0.00 | N/A |

*N = 1

In a second experiment, templated guide RNAs (tgRNAs) targeting the VEGFA locus with varying amounts of 2′-O-methyl-U on the 3′-end were delivered to primary human hepatocytes to assess SpyCas9 indel activity and template-based editing efficiency.

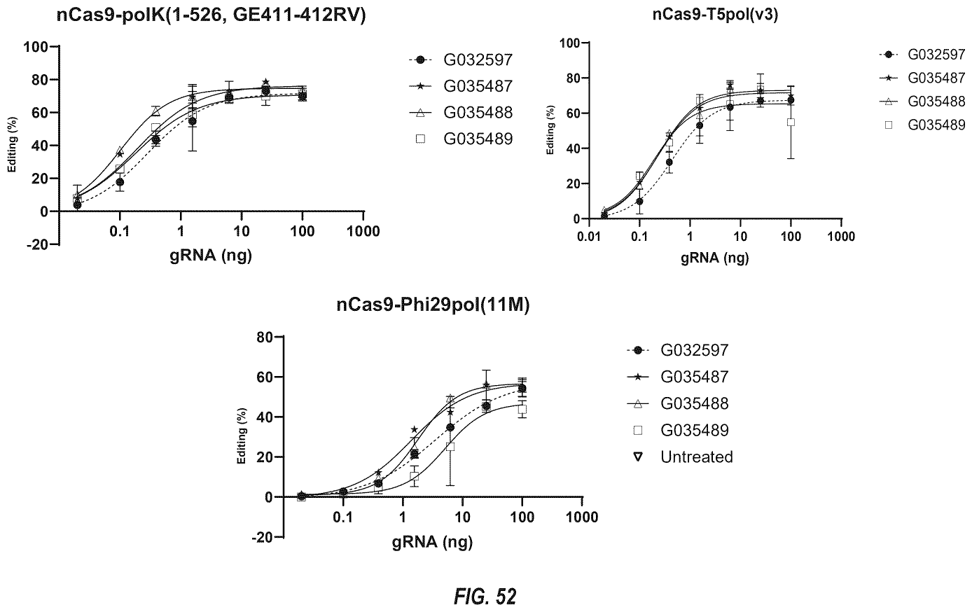

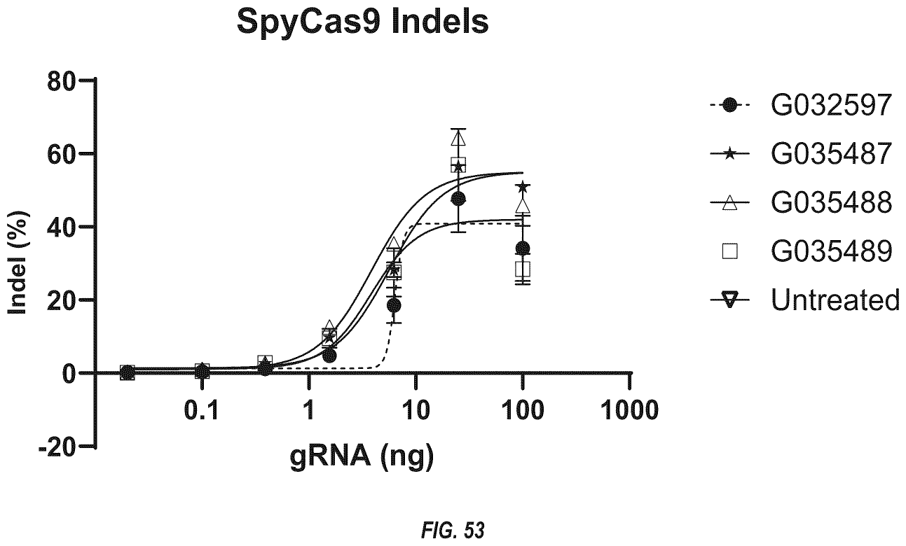

Primary human hepatocytes (Gibco, Lot 8381) were prepared as described in Example 1. After 24 hours from plating, LNPs comprising a single RNA species were used for lipofection. The LNPs were prepared as described in Example 1. The LNPs were made with a molar ratio of 50% Lipid A, 38% cholesterol, 9% DSPC, and 3% PEG2k-DMG, and formulated with a lipid amine to RNA phosphate (N:P) molar ratio of about 6. LNPs were incubated in media containing 10% fetal bovine serum (FBS) at 37° C. for 10 minutes, and were added to the PHH as follows. A LNP comprising a tgRNA, a LNP comprising a Vpx-encoding mRNA (SEQ ID NO: 1501), a LNP comprising a FEN1-encoding mRNA (SEQ ID NO: 1506), and a LNP comprising a mRNA encoding one of a SpyCas9 (SEQ ID NO: 1001), a nCas9-polK(1-526, GE411-412RV), a nCas9-T5pol(v3) (SEQ ID NO: 1222), or a nCas9-Phi29pol(11M) (SEQ ID NO: 1237) were delivered to the cells. The tgRNAs were titrated in an 8-point, 4-fold dose response curve with a top dose of 100 ng. The mRNA cargo was kept constant with the SpyCas9 encoding mRNA or the nCas9-polymerase fusion encoding mRNA at 150 ng/reaction, the Vpx encoding mRNA at 1 ng/reaction, and the FEN1 encoding mRNA at 50 ng/reaction. After delivery and incubation at 37° C., 5% CO₂ for 72 hours, samples were prepared for next-generation sequencing (NGS) and analysis for templated editing efficiency as described in Example 1. Table 58 and FIG. 52 show the editing efficiency results. Table 59 and FIG. 53 show SpyCas9 indel efficiency. The “untreated” controls did not receive any mRNA or gRNA.

TABLE 58
Templated editing efficiency with chemically modified tgRNAs
at VEGFA locus in primary human hepatocytes with FEN1 and Vpx

| | | Number | | | |
|---|---|---|---|---|---|
| | | of 2′-O- | | | |
| | | methyl-U | Guide | | |
| | | on the 3′end | Concen- | Mean | SD |
| | Guide | of the | tration | % | (N = |
| mRNA ID | ID | guide | (ng) | Editing | 2) |
| nCas9-polK(1- | G032597 | 0 | 100 | 69.95 | 0.35 |
| 526, GE411- | (SEQ ID | | 25 | 73.05 | 3.04 |
| 412RV), FEN1, | NO: 530) | | 6.25* | 69.10* | N/A |
| Vpx | | | 1.56 | 54.70 | 18.10 |
| | | | 0.39 | 43.60 | 2.55 |
| | | | 0.10 | 17.80 | 5.52 |
| | | | 0.02 | 3.80 | 1.27 |
| | | | 0* | 0.00* | N/A |
| | G035487 | 1 | 100 | 71.60 | 4.53 |
| | (SEQ ID | | 25 | 78.50 | 0.99 |
| | NO: 537) | | 6.25* | 72.90* | N/A |
| | | | 1.56 | 69.40 | 6.51 |
| | | | 0.39 | 44.15 | 4.60 |
| | | | 0.10* | 34.60* | N/A |
| | | | 0.02 | 7.95 | 7.99 |
| | | | 0* | 0.00* | N/A |
| | G035488 | 2 | 100 | 73.40 | 0.42 |
| | (SEQ ID | | 25 | 78.15 | 1.34 |
| | NO: 538) | | 6.25 | 72.40 | 6.51 |
| | | | 1.56 | 69.65 | 7.43 |
| | | | 0.39 | 60.85 | 2.90 |
| | | | 0.10* | 37.30* | N/A |
| | | | 0.02 | 10.05 | 0.35 |
| | | | 0 | 0.00 | 0.00 |
| | G035489 | 3 | 100 | 71.20 | 3.54 |
| | (SEQ ID | | 25 | 69.90 | 5.66 |
| | NO: 539) | | 6.25 | 67.85 | 1.63 |
| | | | 1.56 | 58.50 | 7.21 |
| | | | 0.39* | 50.90* | N/A |
| | | | 0.10 | 25.70 | 2.12 |
| | | | 0.02* | 7.90* | N/A |
| | | | 0.00 | 0.00 | 0.00 |
| nCas9- | G032597 | 0 | 100 | 67.45 | 1.34 |
| T5pol(v3), | | | 25 | 67.05 | 0.78 |
| FEN1, Vpx | | | 6.25 | 63.40 | 13.29 |
| | | | 1.56 | 53.00 | 10.18 |
| | | | 0.39 | 32.10 | 6.22 |
| | | | 0.10 | 9.80 | 7.07 |
| | | | 0.02 | 1.45 | 0.92 |
| | | | 0 | 0.00 | 0.00 |
| | G035487 | 1 | 100 | 69.85 | 5.30 |
| | | | 25 | 72.85 | 4.03 |
| | | | 6.25 | 76.00 | 2.55 |
| | | | 1.56 | 62.85 | 1.91 |
| | | | 0.39 | 46.65 | 0.21 |
| | | | 0.10* | 20.70* | N/A |
| | | | 0.02 | 3.05 | 0.21 |
| | | | 0* | 0.00* | N/A |
| | G035488 | 2 | 100 | 69.20 | 1.13 |
| | | | 25 | 72.85 | 9.40 |
| | | | 6.25 | 73.75 | 3.89 |
| | | | 1.56 | 58.90 | 11.74 |
| | | | 0.39 | 48.75 | 0.07 |
| | | | 0.10 | 18.60 | 8.06 |
| | | | 0.02* | 5.00* | N/A |
| | | | 0 | 0.00 | 0.00 |
| | G035489 | 3 | 100 | 54.75 | 20.72 |
| | | | 25 | 73.40 | 1.27 |
| | | | 6.25 | 65.15 | 9.12 |
| | | | 1.56* | 67.00* | N/A |
| | | | 0.39 | 43.30 | 5.52 |
| | | | 0.10* | 24.30* | N/A |
| | | | 0.02 | N/A | N/A |
| | | | 0* | 0.00* | N/A |
| nCas9- | G032597 | 0 | 100 | 54.40 | 4.38 |
| Phi29pol(11M), | | | 25 | 45.45 | 3.18 |
| FEN1, Vpx | | | 6.25* | 34.80* | N/A |
| | | | 1.56 | 21.55 | 2.19 |
| | | | 0.39 | 6.80 | 0.71 |
| | | | 0.10* | 2.70* | N/A |
| | | | 0.02 | 0.35 | 0.49 |
| | | | 0 | 0.00 | 0.00 |
| | G035487 | 1 | 100 | 54.85 | 4.60 |
| | | | 25 | 55.95 | 7.42 |
| | | | 6.25 | 42.40 | 7.92 |
| | | | 1.56* | 33.70* | N/A |
| | | | 0.39* | 12.20* | N/A |
| | | | 0.10* | 2.60* | N/A |
| | | | 0.02* | 1.00* | N/A |
| | | | 0 | 0.00 | 0.00 |
| | G035488 | 2 | 100 | 55.00 | 2.69 |
| | | | 25 | 56.20 | 1.56 |
| | | | 6.25* | 49.50* | N/A |
| | | | 1.56 | 24.80 | 4.95 |
| | | | 0.39 | 9.25 | 1.91 |
| | | | 0.10* | 1.50* | N/A |
| | | | 0.02* | 1.20* | N/A |
| | | | 0 | 0.00 | 0.00 |
| | G035489 | 3 | 100 | 43.90 | 4.24 |
| | | | 25 | 45.25 | 0.07 |
| | | | 6.25 | 25.15 | 19.45 |
| | | | 1.56 | 10.40 | 5.23 |
| | | | 0.39 | 4.90 | 3.25 |
| | | | 0.10* | 1.60* | N/A |
| | | | 0.02* | 0.00* | N/A |
| | | | 0 | 0.00 | 0.00 |
| Untreated | N/A | N/A | 0 | 0.00 | 0.00 |

*N = 1 due to technical error

TABLE 59
Indel analysis with chemically modified tgRNAs at the VEGFA locus
in primary human hepatocytes treated with SpyCas9, FEN1 and Vpx.

| | | | Guide | | | |
|---|---|---|---|---|---|---|
| mRNA | Guide | Number of 2′- | Concentration | Mean % | SD | EC50 |
| ID | ID | O-methyl-U | (ng) | Editing | (N = 2) | (ng) |
| SpyCas9, | G032597 | 0 | 100.00 | 34.10 | 8.91 | 6.40 |
| FEN1, | | | 25.00 | 47.70 | 9.19 | |
| Vpx | | | 6.25 | 18.55 | 4.88 | |
| | | | 1.56 | 4.75 | 0.35 | |
| | | | 0.39 | 1.20 | 0.14 | |
| | | | 0.10 | 0.40 | 0.14 | |
| | | | 0.02 | 0.20 | 0.00 | |
| | | | 0.00 | 0.10 | 0.00 | |
| | G035487 | 1 | 100.00 | 50.90 | 1.98 | 5.64 |
| | | | 25.00 | 56.50 | 1.84 | |
| | | | 6.25 | 28.25 | 2.05 | |
| | | | 1.56 | 9.70 | 0.85 | |
| | | | 0.39 | 2.65 | 0.35 | |
| | | | 0.10 | 0.95 | 0.07 | |
| | | | 0.02 | 0.30 | 0.00 | |
| | | | 0.00 | 0.10 | 0.00 | |
| | G035488 | 2 | 100.00 | 45.85 | 5.59 | 3.85 |
| | | | 25.00 | 64.30 | 1.41 | |
| | | | 6.25 | 35.35 | 1.34 | |
| | | | 1.56 | 12.65 | 0.07 | |
| | | | 0.39 | 2.70 | 0.57 | |
| | | | 0.10 | 1.05 | 0.07 | |
| | | | 0.02 | 0.25 | 0.07 | |
| | | | 0.00 | 0.05 | 0.07 | |
| | G035489 | 3 | 100.00 | 28.45 | 4.17 | 3.80 |
| | | | 25.00 | 56.95 | 9.83 | |
| | | | 6.25 | 27.50 | 6.65 | |
| | | | 1.56 | 9.55 | 2.62 | |
| | | | 0.39 | 2.75 | 0.50 | |
| | | | 0.10 | 0.50 | 0.28 | |
| | | | 0.02 | 0.15 | 0.07 | |
| | | | 0.00 | 0.10 | 0.00 | |
| Untreated | N/A | N/A | 0.00 | 0.05 | 0.07 | N/A |

### Example 24—Templated Editing in Primary Human Hepatocytes in the Presence of Enhancers of Template-Based Editing (FEN1, Vpx, MLH1-dn, and MLH1-NTD) with Polymerase K or T5 DNA Polymerase

This Example evaluated the impact of accessory factors (i.e., editing enhancers) on templated editing efficiency. In particular, the editing accessory factors FEN1, Vpx, and MLH1-dn, and MLH1-NTD were assessed in this Example.

Editing accessory factors, FEN1 (SEQ ID NO: 1506), Vpx (SEQ ID NO: 1501), MLH1-dn (SEQ ID NO: 1548), and MLH1-NTD (SEQ ID NO: 1549), were delivered into primary human hepatocytes (PHH) in conjunction with a tgRNA (G032595) comprising a spacer sequence targeting the VEGFA locus and a 3′ extension encoding a GAGG-to-CACT base edit, and either a mRNA encoding polk-nCas9 (SEQ ID NO: 1025) or a mRNA encoding nCas9-T5Pol(D164A,E166A) (SEQ ID NO: 1130). PHH (Gibco, Lot 8381) were prepared as described in Example 1, and plated on a collagen I coated 96-well plate (Stem cell technologies, Cat. 100-036) at a density of 33,000 cells/well. LNPs comprising a single RNA species were used for lipofection. Each LNP contained one of an mRNA encoding an editing enhancer (FEN1, Vpx, MLH1-dn, or MLH1-NTD); an mRNA encoding an nCas9-polymerase fusion protein (polK-nCas9 or nCas9-T5Pol(D164A,E166A)); or a tgRNA (G032595). The LNPs were prepared as described in Example 1. The LNPs were made with a molar ratio of 50% Lipid A, 38% cholesterol, 9% DSPC, and 3% PEG2k-DMG, and formulated with a lipid amine to RNA phosphate (N:P) molar ratio of about 6. Cells were incubated at 37° C., 5% CO₂ for 24 hours prior to treatment with LNPs. LNPs were incubated in media containing 6% fetal bovine serum (FBS) at 37° C. for 10 minutes. Post-incubation, the LNPs were added to the PHH as follows. The guide RNA, and the mRNA encoding the fusion protein (polK-nCas9 ornCas9-T5Pol(D164A,E166A)) were delivered to the cells in a tgRNA:mRNA ratio of 1:2 by weight, at 100 ng total LNP. In addition to the guide RNA and mRNA encoding the fusion protein, an mRNA encoding an editing enhancer (FEN1, Vpx, MLH1-dn, or MLH1-NTD) was delivered via an additional LNP in a 6-point 3-fold dilution series starting at 50 ng. The cells were lysed 72 hours post-treatment for NGS analysis as described in Example 1. Samples were run in duplicate.

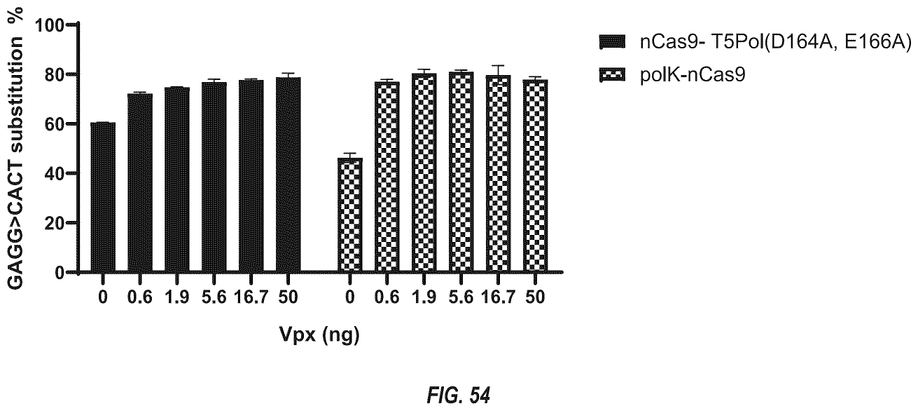

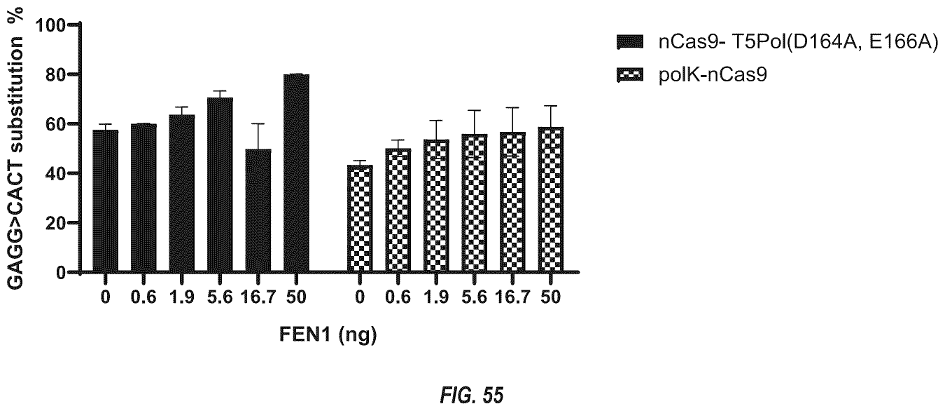

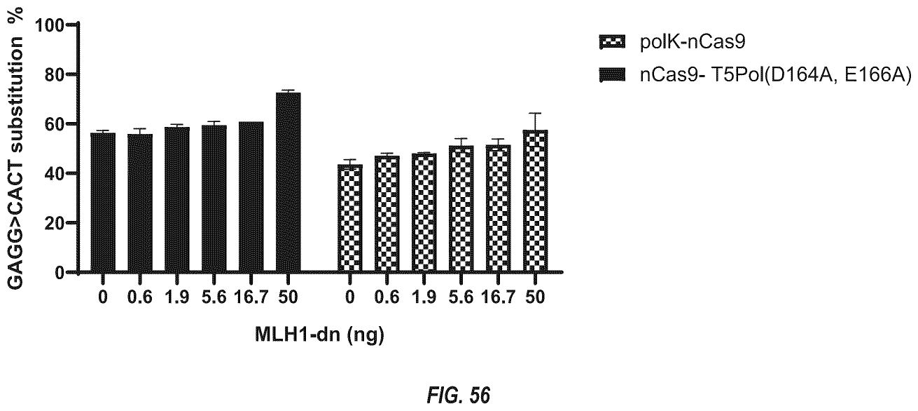

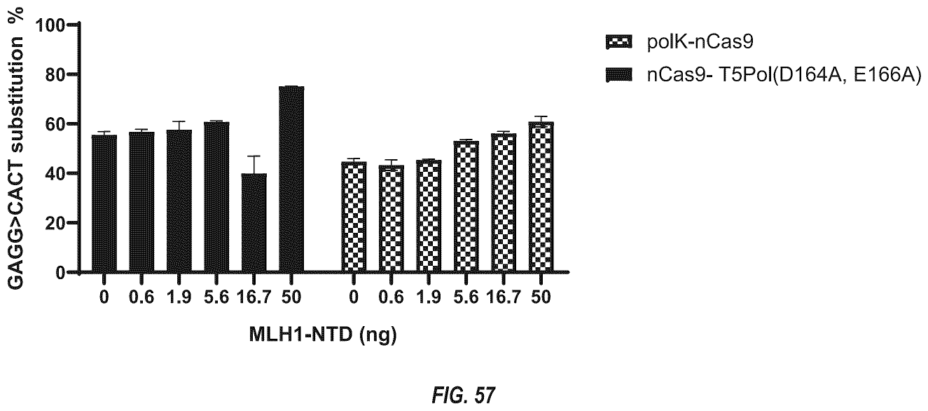

Table 60 and FIG. 54 show editing efficiencies obtained with polk-nCas9 or nCas9-T5Pol(D164A,E166A) in cells treated with Vpx. Table 61 and FIG. 55 show editing efficiencies obtained with polk-nCas9 or nCas9-T5Pol(D164A,E166A) in cells treated with FEN1. Table 62 and FIG. 56 show editing efficiencies obtained with polk-nCas9 or nCas9-T5Pol(D164A,E166A) in cells treated with MLH1-dn. Table 63 and FIG. 57 show editing efficiencies obtained with polk-nCas9 or nCas9-T5Pol(D164A,E166A) in cells treated with MLH1-NTD.

TABLE 60
Templated editing at the VEGFA locus in cells treated with
Vpx, along with PolK-nCas9 or nCa9-T5 T5Pol(D164A, E166A)

| Guide ID | mRNA | Vpx (ng) | Mean editing % | SD |
|---|---|---|---|---|
| G032595 | nCas9- | 50 | 78.80 | 1.70 |
| | T5Pol(D164A, | 16.7 | 77.70 | 0.42 |
| | E166A) (SEQ ID | 5.6 | 76.90 | 1.13 |
| | NO: 1130) | 1.9 | 74.75 | 0.21 |
| | | 0.6 | 72.20 | 0.57 |
| | | 0 | 60.50 | 0.14 |
| | polK-nCas9 | 50 | 77.85 | 1.20 |
| | (SEQ ID NO: | 16.7 | 79.70 | 3.82 |
| | 1025) | 5.6 | 81.05 | 0.64 |
| | | 1.9 | 80.45 | 1.63 |
| | | 0.6 | 77.05 | 0.92 |
| | | 0 | 46.30 | 1.84 |

TABLE 61
Templated editing at the VEGFA locus in cells treated with
FEN1, along with PolK-nCas9 or nCa9-T5 T5Pol(D164A, E166A)

| Guide ID | mRNA | FEN1 (ng) | Mean editing % | SD |
|---|---|---|---|---|
| G032595 | nCas9- | 50 | 79.95 | 0.21 |
| | T5Pol(D164A, | 16.7 | 49.75 | 10.25 |
| | E166A) (SEQ ID | 5.6 | 70.60 | 2.69 |
| | NO: 1130) | 1.9 | 63.70 | 3.11 |
| | | 0.6 | 60.05 | 0.07 |
| | | 0 | 57.60 | 2.26 |
| | polK-nCas9 | 50 | 58.80 | 8.49 |
| | (SEQ ID NO: | 16.7 | 56.75 | 9.83 |
| | 1025) | 5.6 | 55.95 | 9.55 |
| | | 1.9 | 53.70 | 7.64 |
| | | 0.6 | 50.15 | 3.32 |
| | | 0 | 43.40 | 1.70 |

TABLE 62
Templated editing at the VEGFA locus in cells treated with MLH1-
dn, along with PolK-nCas9 or nCa9-T5 T5Pol(D164A, E166A)

| Guide ID | mRNA | MLH1-dn (ng) | Mean editing % | SD |
|---|---|---|---|---|
| G032595 | nCas9- | 50 | 72.60 | 0.99 |
| | T5Pol(D164A, | 16.7* | 60.90* | N/A |
| | E166A) (SEQ ID | 5.6 | 59.45 | 1.48 |
| | NO: 1130) | 1.9 | 58.70 | 1.13 |
| | | 0.6 | 55.85 | 2.19 |
| | | 0 | 56.35 | 0.92 |
| | polK-nCas9 | 50 | 57.55 | 6.72 |
| | (SEQ ID NO: | 16.7 | 51.50 | 2.40 |
| | 1025) | 5.6 | 51.20 | 2.83 |
| | | 1.9 | 48.05 | 0.35 |
| | | 0.6 | 47.10 | 0.99 |
| | | 0 | 43.60 | 1.98 |

*N = 1

TABLE 63
Templated editing at the VEGFA locus in cells treated with MLH1-
NTD, along with PolK-nCas9 or nCa9-T5 T5Pol(D164A, E166A)

| Guide ID | mRNA | MLH1-NTD (ng) | Mean editing % | SD |
|---|---|---|---|---|
| G032595 | nCas9- | 50 | 75.15 | 0.07 |
| | T5Pol(D164A, | 16.7 | 39.95 | 7.00 |
| | E166A) (SEQ | 5.6 | 60.75 | 0.49 |
| | ID NO: 1130) | 1.9 | 57.65 | 3.32 |
| | | 0.6 | 56.75 | 1.06 |
| | | 0 | 55.55 | 1.34 |
| | polK-nCas9 | 50 | 60.90 | 2.12 |
| | (SEQ ID NO: | 16.7 | 56.15 | 0.78 |
| | 1025) | 5.6 | 53.10 | 0.57 |
| | | 1.9 | 45.40 | 0.28 |
| | | 0.6 | 43.30 | 2.12 |
| | | 0 | 44.70 | 1.27 |

### Example 25—Templated Editing in Primary Human Hepatocytes in the Presence of Enhancers of Template-Based Editing (FEN1 and Vpx) with Polymerase Kappa or T5 DNA Polymerase

This Example evaluated the impact of accessory factors (i.e., editing enhancers) on templated editing efficiency. The editing accessory factors FEN1 and Vpx were assessed in this Example.

Editing accessory factors, FEN1 (SEQ ID NO: 1506) and Vpx (SEQ ID NO: 1501), were delivered into primary human hepatocytes (PHH) in conjunction with a tgRNA (G032595) comprising a spacer sequence targeting the VEGFA locus and a 3′ extension encoding a GAGG-to-CACT base edit, and either a mRNA encoding polk-nCas9 (SEQ ID NO: 1025) or a mRNA encoding nCas9-T5Pol(D164A,E166A) (SEQ ID NO: 1130).

PHH (Gibco, Lot 8381) were prepared as described in Example 1, and plated on a collagen I coated 96-well plate (Stem cell technologies, Cat. 100-036) at a density of 33,000 cells/well. LNPs comprising a single RNA species were used for lipofection. Each LNP contained one of an mRNA encoding an editing enhancer (FEN1 or Vpx); an mRNA encoding an nCas9-polymerase fusion protein (polK-nCas9 or nCas9-T5Pol(D164A, E166A)); or a tgRNA (G032595). The LNPs were prepared as described in Example 1. The LNPs were made with a molar ratio of 50% Lipid A, 38% cholesterol, 9% DSPC, and 3% PEG2k-DMG, and formulated with a lipid amine to RNA phosphate (N:P) molar ratio of about 6. Cells were incubated at 37° C., 5% CO₂ for 24 hours prior to treatment with LNPs. LNPs were incubated in media containing 10% fetal bovine serum (FBS) at 37° C. for 10 minutes. Post-incubation, the LNPs were added to the PHH as follows. The guide_RNA and the mRNA encoding the fusion protein (polK-nCas9 or nCas9-T5Pol(D164A, E166A)) were delivered to the cells in a tgRNA:mRNA ratio of 1:2 by weight, in a 5-point 3-fold dilution series starting at 200 ng total LNP. For each dilution of the guide RNA and mRNA encoding the fusion protein, Vpx was added at a fixed concentration of 1 ng on its own or combined with 2, 10, or 50 ng of FEN1. For comparison, cells were also treated with 2, 10, or 50 ng of FEN1 without Vpx. The cells were lysed 72 hours post-treatment for NGS analysis as described in Example 1. Samples were run in duplicate unless otherwise indicated.

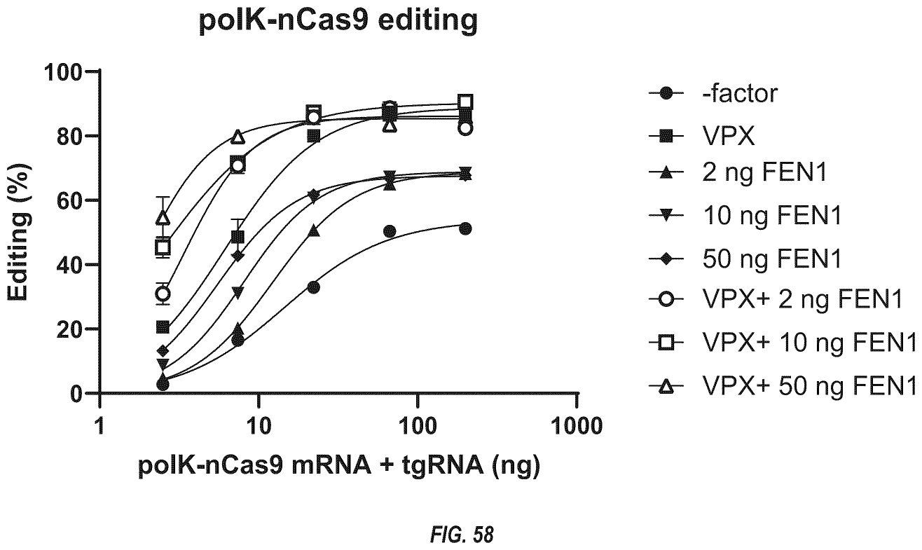

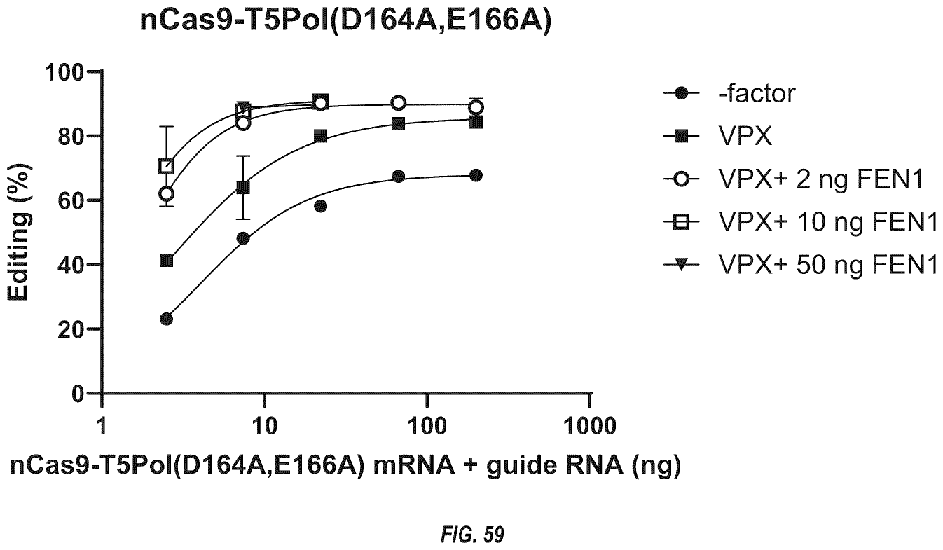

FIG. 58 and Table 64 show editing efficiencies obtained with polk-nCas9 at the VEGFA locus in cells treated with FEN1, Vpx, or a combination of Vpx and FEN1. FIG. 59 and Table 65 show editing efficiencies obtained with nCas9-T5Pol(D164A,E166A) at the VEGFA locus in cells treated with FEN1, Vpx, or a combination of Vpx and FEN1.

TABLE 64
Templated editing at the VEGFA locus in cells treated with polK-
nCas9, and Vpx, FEN1, or a combination of Vpx and FEN1. EC50 values
are indicated for conditions with the highest editing.

| | | polK-nCas9 mRNA and guide RNA (2:1 ratio) (ng) | | | | | |
|---|---|---|---|---|---|---|---|
| | | 200 | 66.7 | 22.2 | 7.4 | 2.5 | 0 |
| -factor | Editing % | 51.20* | 50.40* | 32.95 | 16.50 | 2.80 | 0.00 |
| | SD | N/A | N/A | 1.48 | 0.14 | 0.14 | 0.00 |
| 1 ng Vpx | Editing % | 86.55 | 87.10 | 80.00 | 48.60 | 20.65 | 0.00 |
| | SD | 2.05 | 2.12 | 1.70 | 5.52 | 1.63 | 0.00 |
| | EC50 (ng) | 6.14 | | | | | |
| 2 ng FEN1 | Editing % | 68.40* | 65.10* | 50.80* | 20.30* | 4.70* | 0.00 |
| | SD | N/A | N/A | N/A | N/A | N/A | 0.00 |
| 10 ng FEN1 | Editing % | 68.40* | 67.20* | 60.70* | 31.00* | 8.70* | 0.00 |
| | SD | N/A | N/A | N/A | N/A | N/A | 0.00 |
| 50 ng FEN1 | Editing % | 67.80* | 66.90* | 61.70* | 42.80* | 13.20* | 0.00 |
| | SD | N/A | N/A | N/A | N/A | N/A | 0.00 |
| 1 ng Vpx + | Editing % | 82.45 | 88.60 | 85.75 | 70.80 | 30.95 | 0.00* |
| 2 ng FEN1 | SD | 0.64 | 1.27 | 2.19 | 2.40 | 3.32 | N/A |
| | EC50 (ng) | 3.37 | | | | | |
| 1 ng Vpx + | Editing % | 90.60 | 87.60 | 87.30* | 71.50* | 45.40 | 0.00 |
| 10 ng FEN1 | SD | 1.41 | 2.97 | N/A | N/A | 3.25 | 0.00 |
| | EC50 (ng) | 2.49 | | | | | |
| 1 ng Vpx + | Editing % | 85.90* | 83.50* | 86.50* | 79.90* | 54.70 | 0.00 |
| 50 ng FEN1 | SD | N/A | N/A | N/A | N/A | 6.36 | 0.00 |

| | |
|---|---|
| EC50 (ng) | 1.87 |

*N = 1

TABLE 65
Templated editing at the VEGFA locus in cells treated with nCas9-T5Pol(D164A,
E166A), and Vpx, FEN1, or a combination of Vpx and FEN1. EC50 values
are indicated for conditions with the highest editing

| | | nCas9-T5Pol(D164A, E166A) mRNA and guide RNA (2:1 ratio) (ng) | | | | | |
|---|---|---|---|---|---|---|---|
| | | 200 | 66.7 | 22.2 | 7.4 | 2.5 | 0 |
| -factor | Editing % | 67.75 | 67.45 | 58.20* | 48.20 | 23.15 | 0.00 |
| | SD | 1.91 | 0.92 | N/A | 0.42 | 0.21 | 0.00 |
| 1 ng Vpx | Editing % | 84.40 | 83.85 | 80.00 | 63.95 | 41.40* | 0.00* |
| | SD | 0.99 | 0.21 | 0.99 | 9.83 | N/A | N/A |
| | EC50 (ng) | 2.75 | | | | | |
| 1 ng Vpx + | Editing % | 88.85 | 90.30 | 90.20* | 84.00* | 62.05 | 0.00 |
| 2 ng FEN1 | SD | 2.76 | 1.56 | N/A | N/A | 0.07 | 0.00 |
| | EC50 (ng) | 1.60 | | | | | |
| 1 ng Vpx + | Editing % | | | 90.70* | 87.60* | 70.50 | 0.00 |
| 10 ng FEN1 | SD | | | N/A | N/A | 12.45 | 0.00 |
| | EC50 (ng) | 1.27 | | | | | |
| 1 ng Vpx+ | Editing % | | | 89.80* | 88.80* | | |
| 50 ng FEN1 | SD | | | N/A | N/A | | |

### Example 26—Dose Response Experiment with VEGFA Guide RNA in Huh7 Cells and Primary Human Hepatocytes Using Fusion Proteins Containing Polymerase K Orthologs

This Example evaluated the impact of different polymerase K orthologs on templated editing efficiency. Human, mouse, and cynomolgus polymerase K orthologs were assessed in the context of single polypeptide fusion proteins encoding a SpyCas9 nickase (nCas9) moiety and a DNA-dependent DNA polymerase moiety.

mRNAs encoding fusion proteins comprising a SpyCas9 nickase (nCas9) moiety and a DNA-dependent DNA polymerase moiety were delivered to Huh7 cells and to primary human hepatocytes (PHH) with a template guide RNA (tgRNA, G032596; SEQ ID NO: 417) comprising a spacer sequence targeting the VEGFA locus and a 3′ extension sequence comprising a DNA polymerase recruitment site (i.e., DNA polymerase recruiting sequence; DRS) and a DNA polymerase template encoding a GAGG-to-CACT 3-nt substitution mutation. Each mRNA encoded one of the following fusion proteins: nCas9-POLK(human, 1-526,GE411-412RV) (SEQ ID NO: 1232), nCas9-polK(mouse, 1-525,GE410-411RV) (SEQ ID NO: 1246), and nCas9-polK (cyno, 1-526,GE411-412RV) (SEQ ID NO: 1241).

Huh7 cells (Japanese Collection of Research Biosources (JCRB) Cell Bank, lot #JCRB0403) were prepared as described in Example 1, plated at a density of 15,000 cells/well in a 96-well plate format and incubated at 37° C. and 5% CO₂ atmosphere for 24 hours. Nucleic acid components were delivered using Lipofectamine™ MessengerMAX™ Reagent (Thermo Fisher, Cat. LMRNA001) according to the manufacturer's protocol. Huh7 cells were transfected with mRNA encoding one of the fusion proteins and tgRNA targeting VEGFA (G032596) in a tgRNA:mRNA ratio of 1:2 by weight. Transfection was done in a six-point, two-fold dilution series, where the highest dose was 300 ng total RNA, including 200 ng of mRNA and 100 ng of tgRNA. Samples were run in triplicate.

Primary human hepatocytes (Gibco, lot 8381) were prepared as described in Example 1, and plated at a density of 33,000 cells/well. After 24 hours of incubation at 37° C., 5% CO₂, cells were lipofected as described in Example 1, with mRNA encoding one of the fusion proteins and tgRNA targeting VEGFA (G032596). Guide nucleic acids and mRNAs were prepared in separate lipoplexes before mixing and diluting in a six-point, two-fold dilution series. The highest dose was 300 ng total RNA. Samples were run in triplicate.

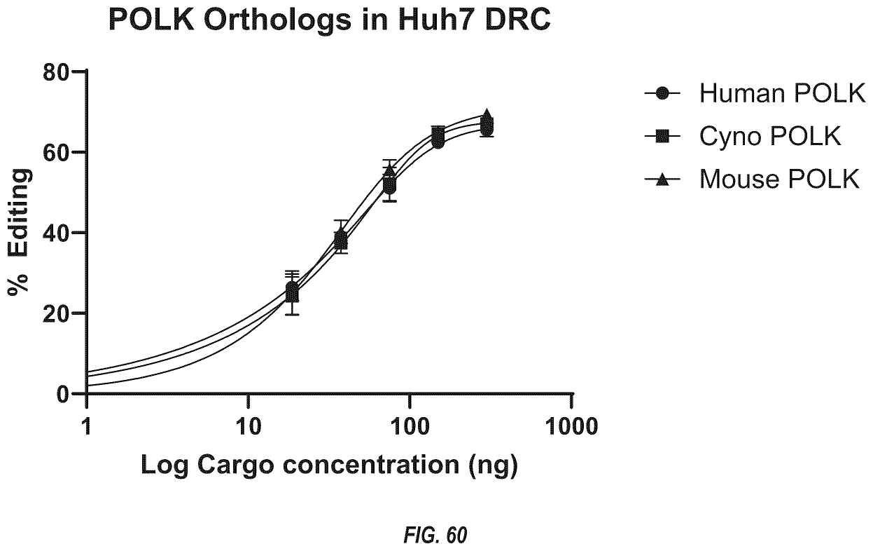

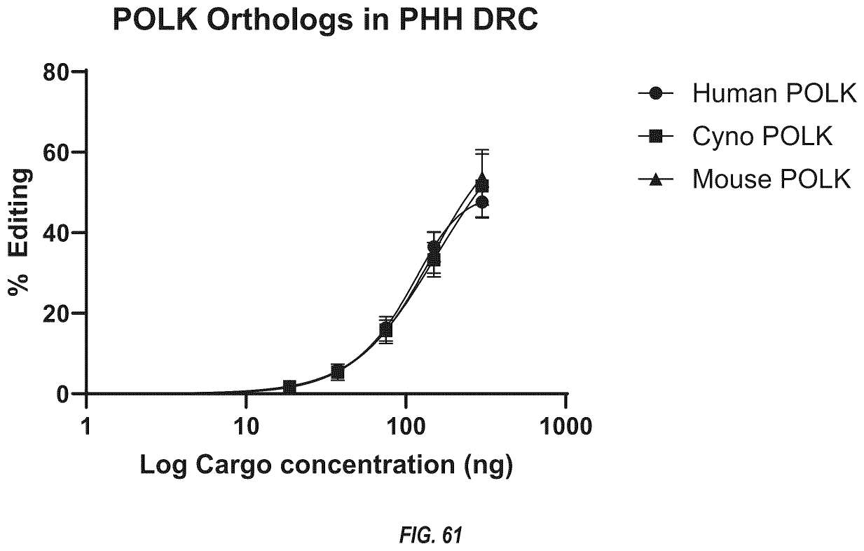

Cells were lysed 3 days post-treatment for NGS analysis as described in Example 1 to measure editing at the VEGFA locus. Mean percent templated editing with standard deviation (SD) are shown in Table 66 and FIG. 60 for Huh7 cells and in Table 67 and FIG. 61 for PHH cells.

TABLE 66
Templated editing at the VEGFA locus in Huh7 cells.

| | | Total | Mean % | SD |
|---|---|---|---|---|
| mRNA ID | Guide ID | RNA (ng) | Editing | (N = 3) |
| nCas9-POLK(1- | G032596 | 300 | 65.57 | 1.64 |
| 526, GE411-412RV) | | 150 | 62.43 | 1.19 |
| (SEQ ID NO: 1232) | | 75 | 51.07 | 3.43 |
| | | 37.5 | 39.03 | 0.75 |
| | | 18.8 | 26.40 | 3.36 |
| | | 0 | 0.00 | 0.00 |
| nCas9-polK(mouse, 1- | | 300 | 69.33 | 0.80 |
| 525, GE410-411RV) | | 150 | 65.07 | 1.06 |
| (SEQ ID NO: 1246) | | 75 | 55.67 | 2.46 |
| | | 37.5 | 40.23 | 2.89 |
| | | 18.8 | 25.07 | 5.40 |
| | | 0 | 0.00 | 0.00 |
| nCas9-polK(cyno, 1- | | 300 | 67.03 | 0.55 |
| 526, GE411-412RV | | 150 | 64.50 | 1.91 |
| (SEQ ID NO: 1241 | | 75 | 52.07 | 4.14 |
| | | 37.5 | 37.47 | 2.65 |
| | | 18.8 | 24.27 | 4.76 |
| | | 0 | 0.00 | 0.00 |

TABLE 67
Templated editing at the VEGFA locus in PHH cells.

| | Guide | Total RNA | Mean % | |
|---|---|---|---|---|
| mRNA ID | ID | (ng) | Editing | SD |
| nCas9-POLK(1- | G032596 | 300 | 47.65* | 3.75* |
| 526, GE411-412RV) | | 150 | 36.50 | 3.70 |
| (SEQ ID NO: 1232) | | 75 | 16.40* | 0.99* |
| | | 37.5 | 5.87 | 1.31 |
| | | 18.8 | 1.73 | 0.64 |
| | | 0 | 0.00* | 0.00* |
| nCas9-polK(mouse, 1- | | 300 | 53.77 | 6.86 |
| 525, GE410-411RV) | | 150 | 35.03 | 5.06 |
| (SEQ ID NO: 1246) | | 75 | 15.67 | 2.63 |
| | | 37.5 | 5.70 | 1.08 |
| | | 18.8 | 1.83 | 0.21 |
| | | 0 | 0.05* | 0.07* |
| nCas9-polK(cyno, 1- | | 300 | 51.60 | 7.98 |
| 526, GE411-412RV | | 150 | 33.30 | 4.25 |
| (SEQ ID NO: 1241) | | 75 | 15.80 | 3.39 |
| | | 37.5 | 5.37 | 1.99 |
| | | 18.8 | 1.77 | 0.59 |
| | | 0 | 0.00 | 0.00 |

*N = 2

### Example 27—Templated Editing in Primary Human Hepatocytes with T5 DNA Polymerase in the Presence of FEN1 Variants and Orthologs

This Example evaluated the impact of different variants and orthologs of the FEN1 accessory factor (i.e., editing enhancer) on templated editing efficiency. In particular, human, cynomolgus, and Chrysemys picta bellii FEN1 orthologs were assessed. Additionally assessed were FEN1 fusions including a heterologous nuclear localization signal (NLS), an EI sequence, or a KI sequence. The KI and EI sequences are coiled coil heterodimerization domains capable of self-assembly into stable heterodimers to promote polymerase-FEN1 heterodimer formation.

An mRNA encoding one of variants of editing accessory factor FEN1, including a human FEN1 (hFEN1, SEQ ID NO: 1506), a FEN1 construct w/o a heterologous NLS sequence (hFEN1, SEQ ID NO: 1472), a cynomolgus monkey FEN1 ortholog (cynoFEN1, SEQ ID NO: 1477), a Chrysemys picta bellii FEN1 ortholog (pictaFEN1, SEQ ID NO: 1482) and a human FEN1 fused to either a KI (hFEN1-KI, SEQ ID NO: 1487) or an EI (hFEN1-EI, SEQ ID NO: 1492) sequence, were delivered into primary human hepatocytes (PHH) in conjunction with a tgRNA targeting VEGFA locus (G032596), and a mRNA encoding either one of nCas9-T5pol(v3) (SEQ ID NO: 1222), a nCas9-T5Pol fusion protein comprising a KI sequence (nCas9-T5-KI, SEQ ID NO: 1256) or a nCas9-T5Pol fusion protein comprising an EI sequence (nCas9-T5-EI, SEQ ID NO: 1251).

Dilution Series of Cas9-DNA Polymerase and tgRNA

PHH (Gibco, Lot 8381) were prepared as described in Example 1, and plated on a collagen I coated 96-well plate (StemCell Technologies, Cat. 100-0366) at a density of 33,000 cells/well. LNPs comprising a single RNA species were used for lipofection. Each LNP contained one of an mRNA encoding a FEN1 variant or ortholog (hFEN1, hFEN1 w/o a heterologous NLS, cynoFEN1, pictaFEN1, hFEN1-KI, or hFEN1-EI); an mRNA encoding a nCas9-polymerase fusion (nCas9-T5pol(v3), nCas9-T5-KI, or nCas9-T5-KI); or a tgRNA (G032596). The LNPs were prepared as described in Example 1. The LNPs were made with a molar ratio of 50% Lipid A, 38% cholesterol, 9% DSPC, and 3% PEG2k-DMG, and formulated with a lipid amine to RNA phosphate (N:P) molar ratio of about 6. Cells were incubated at 37° C., 5% CO₂ for 24 hours prior to treatment with LNPs. LNPs were incubated in media containing 10% fetal bovine serum (FBS) at 37° C. for 10 minutes. Post-incubation, the LNPs were added to the PHH as follows. The mRNA encoding a FEN1 variant or ortholog (hFEN1, hFEN1 w/o a heterologous NLS, cynoFEN1, pictaFEN1, hFEN1-KI, or hFEN1-EI) was delivered to the cells at a dose of 100 ng. In addition to the mRNA encoding the FEN1 variant or ortholog, LNPs comprising the guide RNA, and the mRNA encoding the nCas9-polymerase fusion nCas9-T5pol(v3), nCas9-T5-KI, or nCas9-T5-KI) were delivered to the cells in a tgRNA:mRNA ratio of 1:2 by weight, in an 8-point 3-fold dilution series starting at 200 ng total LNP. Control groups (“no FEN1”) consisted of cells transfected with the tgRNA and the mRNA encoding one of the nCas9-polymerase fusions without any editing accessory factor. The cells were lysed 72 hours post-treatment for NGS analysis as described in Example 1. Samples were run in duplicate.

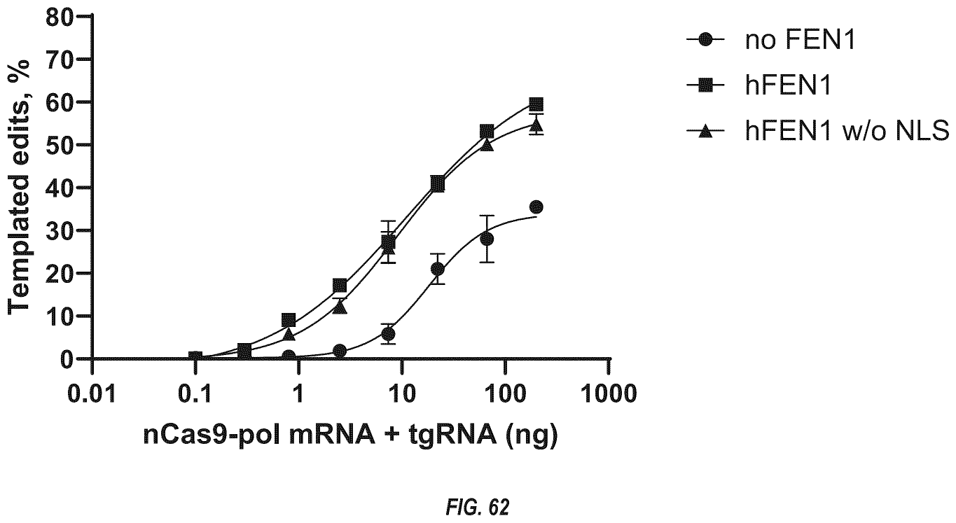

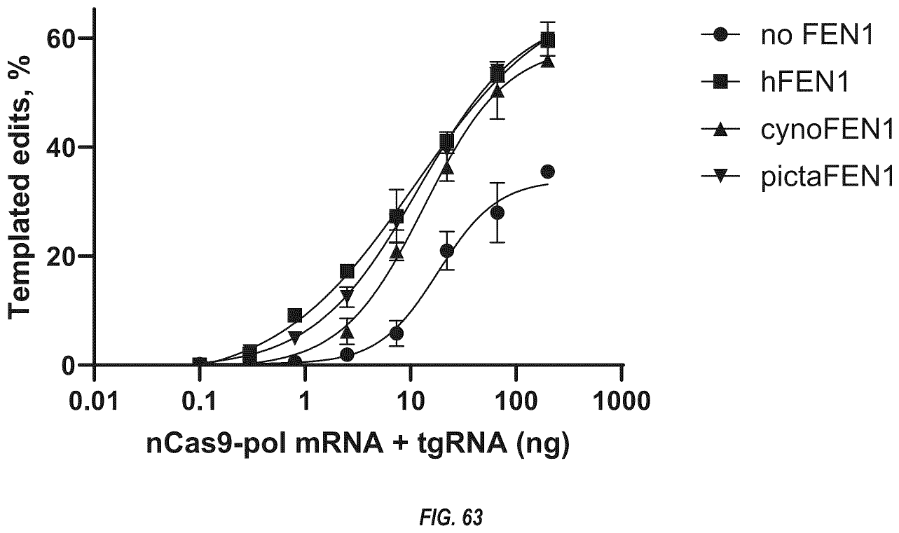

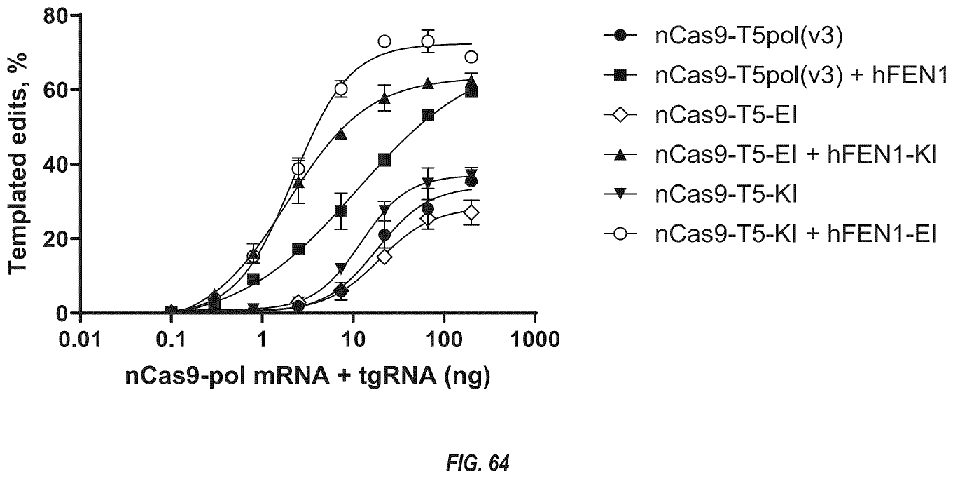

Table 68 and FIG. 62 show editing efficiencies in cells treated with human FEN1, with or without an NLS sequence, along with nCas9-T5pol(v3). Table 69 and FIG. 63 show editing efficiencies in cells treated with hFEN1, cyno FEN1, or picta FEN1, in combination with nCas9-T5pol(v3). Table 70 and FIG. 64 show editing efficiencies in cells treated with hFEN1, hFEN1-KI, or hFEN1-EI, along with one of nCas9-T5pol(v3), or with nCas9-T5-EI or nCas9-T5-KI with the hFEN1 having the coiled coil heterodimerization domain for heterodimer formation with the polymerase, i.e., nCas9-T5-EI with hFEN1-KI; and nCas9-T5-KI with hFEN1-EI as shown in the tables below.

TABLE 68
Templated editing at the VEGFA locus in PHH treated
with one of hFEN1 or hFEN1 w/o a heterologous
NLS, in combination with nCas9-T5pol(v3).

| | | Total (ng) tgRNA + | | |
|---|---|---|---|---|
| | Guide | nCas9-polymerase | Mean % | |
| FEN1 variant | ID | mRNA | Editing | SD |
| no FEN1 | G032596 | 200 | 35.50* | N/A |
| | | 66.7 | 28.00 | 5.52 |
| | | 22.2 | 21.00 | 3.54 |
| | | 7.4 | 5.85 | 2.33 |
| | | 2.5 | 1.90 | 0.14 |
| | | 0.8 | 0.55 | 0.07 |
| | | 0.3 | 0.50 | 0.00 |
| | | 0.1 | 0.20* | N/A |
| hFEN1 (SEQ ID | | 200 | 59.50 | 0.85 |
| NO: 1506) | | 66.7 | 53.25 | 1.34 |
| | | 22.2 | 41.15 | 1.63 |
| | | 7.4 | 27.35 | 4.88 |
| | | 2.5 | 17.25 | 0.78 |
| | | 0.8 | 9.10* | N/A |
| | | 0.3 | 2.10 | 0.85 |
| | | 0.1 | 0.10 | 0.14 |
| hFEN1 w/o | | 200 | 54.80 | 2.40 |
| heterologous NLS | | 66.7 | 50.15 | 0.07 |
| (SEQ ID NO: | | 22.2 | 40.50 | 1.27 |
| 1472) | | 7.4 | 26.00 | 3.68 |
| | | 2.5 | 12.45 | 1.77 |
| | | 0.8 | 5.90 | 0.71 |
| | | 0.3 | 1.70 | 0.71 |
| | | 0.1 | 0.15 | 0.21 |

*N = 1

TABLE 69
Templated editing at the VEGFA locus in PHH
treated with one of hFEN1, cynoFEN1, or pictaFEN1,
in combination with nCas9-T5pol(v3).

| | | Total (ng) tgRNA + | | |
|---|---|---|---|---|
| | Guide | nCas9-polymerase | Mean % | |
| FEN1 variant | ID | mRNA | Editing | SD |
| no FEN1 | G032596 | 200 | 35.50* | N/A |
| | | 66.7 | 28.00 | 5.52 |
| | | 22.2 | 21.00 | 3.54 |
| | | 7.4 | 5.85 | 2.33 |
| | | 2.5 | 1.90 | 0.14 |
| | | 0.8 | 0.55 | 0.07 |
| | | 0.3 | 0.50 | 0.00 |
| | | 0.1 | 0.20* | N/A |
| hFEN1 (SEQ ID | | 200 | 59.50 | 0.85 |
| NO: 1506 | | 66.7 | 53.25 | 1.34 |
| | | 22.2 | 41.15 | 1.63 |
| | | 7.4 | 27.35 | 4.88 |
| | | 2.5 | 17.25 | 0.78 |
| | | 0.8 | 9.10* | N/A |
| | | 0.3 | 2.10 | 0.85 |
| | | 0.1 | 0.10 | 0.14 |
| cynoFEN1 (SEQ | | 200 | 55.95 | 0.92 |
| ID NO: 1477) | | 66.7 | 50.40 | 5.23 |
| | | 22.2 | 36.30 | 2.55 |
| | | 7.4 | 20.90 | 1.70 |
| | | 2.5 | 6.20 | 2.40 |
| | | 0.8 | no data | no data |
| | | 0.3 | 0.40* | N/A |
| | | 0.1 | 0.10 | 0.14 |
| pictaFEN1 (SEQ | | 200 | 59.80 | 3.11 |
| ID NO: 1482) | | 66.7 | 54.00 | 0.85 |
| | | 22.2 | 39.50* | N/A |
| | | 7.4 | 26.25 | 1.48 |
| | | 2.5 | 12.50 | 1.84 |
| | | 0.8 | 4.90 | 0.14 |
| | | 0.3 | 2.50 | 0.14 |
| | | 0.1 | 0.00 | 0.00 |

*N = 1

TABLE 70
Templated editing at the VEGFA locus in PHH treated with
one of hFEN1, hFEN1-KI, or hFEN1-EI, in combination with
one of nCas9-T5pol(v3), nCas9-T5-EI, or nCas9-T5-KI.

| | | | Total (ng) | | |
|---|---|---|---|---|---|
| | | | tgRNA + | | |
| | | nCas9- | nCas9- | Mean | |
| | Guide | polymerase | polymerase | % | |
| FEN1 variant | ID | mRNA | mRNA | Editing | SD |
| no FEN1 | G032596 | nCas9- | 200 | 35.50* | N/A |
| | | T5pol(v3) | 66.7 | 28.00 | 5.52 |
| | | | 22.2 | 21.00 | 3.54 |
| | | | 7.4 | 5.85 | 2.33 |
| | | | 2.5 | 1.90 | 0.14 |
| | | | 0.8 | 0.55 | 0.07 |
| | | | 0.3 | 0.50 | 0.00 |
| | | | 0.1 | 0.20* | N/A |
| hFEN1 (SEQ | | | 200 | 59.50 | 0.85 |
| ID NO: 1506) | | | 66.7 | 53.25 | 1.34 |
| | | | 22.2 | 41.15 | 1.63 |
| | | | 7.4 | 27.35 | 4.88 |
| | | | 2.5 | 17.25 | 0.78 |
| | | | 0.8 | 9.10* | N/A |
| | | | 0.3 | 2.10 | 0.85 |
| | | | 0.1 | 0.10 | 0.14 |
| no FEN1 | | nCas9-T5-EI | 200 | 27.00 | 3.39 |
| | | | 66.7 | 25.40 | 1.41 |
| | | | 22.2 | 15.10 | 0.00 |
| | | | 7.4 | 6.00 | 0.99 |
| | | | 2.5 | 2.90* | N/A |
| | | | 0.8 | 0.40* | N/A |
| | | | 0.3 | 0.40 | 0.00 |
| | | | 0.1 | 0.15 | 0.21 |
| hFEN1-KI | | | 200 | 62.50 | 1.98 |
| (SEQ ID | | | 66.7 | 61.85 | 0.49 |
| NO: 1487) | | | 22.2 | 57.85 | 3.46 |
| | | | 7.4 | 48.30 | 0.28 |
| | | | 2.5 | 35.20 | 5.80 |
| | | | 0.8 | 16.05 | 2.62 |
| | | | 0.3 | 5.05 | 0.49 |
| | | | 0.1 | 0.00 | 0.00 |
| no FEN1 | | nCas9-T5-KI | 200 | 37.05 | 2.05 |
| | | | 66.7 | 34.75 | 4.31 |
| | | | 22.2 | 27.50 | 2.55 |
| | | | 7.4 | 11.80* | N/A |
| | | | 2.5 | 2.85 | 1.34 |
| | | | 0.8 | 1.05 | 0.07 |
| | | | 0.3 | 1.80 | 1.70 |
| | | | 0.1 | 0.10 | 0.14 |
| hFEN1-EI | | | 200 | 68.85 | 1.06 |
| (SEQ ID | | | 66.7 | 73.05 | 3.04 |
| NO: 1492) | | | 22.2 | 73.00 | 0.99 |
| | | | 7.4 | 60.25 | 2.19 |
| | | | 2.5 | 38.75 | 2.90 |
| | | | 0.8 | 15.30* | N/A |
| | | | 0.3 | 3.85 | 0.49 |
| | | | 0.1 | 0.00 | 0.00 |

*N = 1

### Dilution Series of FEN1

In another experiment, human FEN1 (hFEN1, SEQ ID NO: 1506), hFEN1-KI (SEQ ID NO: 1487), or hFEN1-EI (SEQ ID NO: 1492) sequence, was delivered into primary human hepatocytes (PHH) in conjunction with a tgRNA targeting VEGFA (G032596), and a mRNA encoding one of nCas9-T5pol(v3) (SEQ ID NO: 1222), nCas9-T5-KI (SEQ ID NO: 1256), or nCas9-T5-EI (SEQ ID NO: 1251), with the hFEN1 having the coiled coil heterodimerization domain for heterodimer formation with the polymerase, i.e., nCas9-T5-EI with hFEN1-KI; and nCas9-T5-KI with hFEN1-EI as shown in the tables below.

PHH (Gibco, Lot 8381) were prepared as described in Example 1, and plated on a collagen I coated 96-well plate (StemCell Technologies, Cat. 100-0366) at a density of 33,000 cells/well. LNPs comprising a single RNA species were used for lipofection. Each LNP contained one of an mRNA encoding a FEN1 variant (hFEN1, hFEN1-KI, or hFEN1-EI); an mRNA encoding a nCas9-polymerase fusion (nCas9-T5pol(v3), nCas9-T5-KI, or nCas9-T5-KI); or a tgRNA (G032596). The LNPs were prepared as described in Example 1. The LNPs were made with a molar ratio of 50% Lipid A, 38% cholesterol, 9% DSPC, and 3% PEG2k-DMG, and formulated with a lipid amine to RNA phosphate (N:P) molar ratio of about 6. Cells were incubated at 37° C., 5% CO₂ for 24 hours prior to treatment with LNPs. LNPs were incubated in media containing 10% fetal bovine serum (FBS) at 37° C. for 10 minutes. Post-incubation, the LNPs were added to the PHH as follows. For each well, an LNP comprising a mRNA encoding a nCas9-polymerase fusion (nCas9-T5pol(v3), nCas9-T5-KI, or nCas9-T5-KI) and an LNP comprising the tgRNA were delivered at a total RNA amount (tgRNA+mRNA) of 10 ng, with a mass ratio of 1:2 tgRNA:mRNA. In addition to the tgRNA and the mRNA encoding the nCas9-polymerase fusion, a LNP comprising a FEN1 variant (hFEN1, hFEN1-KI, or hFEN1-EI) was delivered to the cells in a 6-point 2-fold dilution series starting at 25 ng. The cells were lysed 72 hours post-treatment for NGS analysis as described in Example 1. Samples were run in quadruplicate.

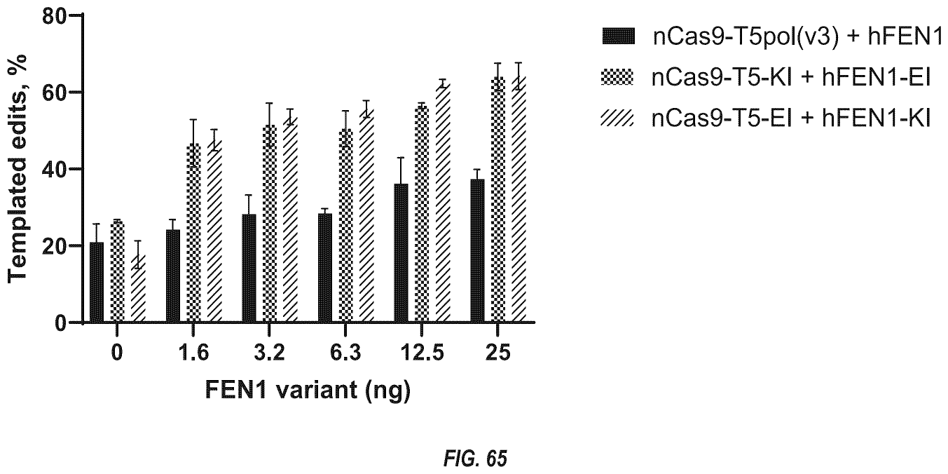

Table 71 and FIG. 65 show editing efficiencies in cells treated with hFEN1, hFEN1-KI, or hFEN1-EI, along with one of nCas9-T5pol(v3), nCas9-T5-KI, or nCas9-T5-KI.

TABLE 71
Templated editing at the VEGFA locus in PHH treated with
one of hFEN1, hFEN1-KI, or hFEN1-EI, in combination with
one of nCas9-T5pol(v3), nCas9-T5-EI, or nCas9-T5-KI.

| | | nCas9- | | | |
|---|---|---|---|---|---|
| | Guide | polymerase | FEN1 | Mean % | |
| FEN1 variant | ID | mRNA | (ng) | Editing | SD |
| hFEN1 (SEQ | G032596 | nCas9-T5pol(v3) | 25 | 37.27** | 2.60** |
| ID NO: 1506) | | | 12.5 | 36.18 | 6.77 |
| | | | 6.3 | 28.43** | 1.29** |
| | | | 3.2 | 28.13** | 5.08** |
| | | | 1.6 | 24.23** | 2.57** |
| | | | 0 | 20.90 | 4.83 |
| hFEN1-EI | | nCas9-T5-KI | 25 | 64.00* | 3.54* |
| (SEQ ID NO: | | | 12.5 | 56.50* | 0.71* |
| 1492) | | | 6.3 | 50.50* | 4.67* |
| | | | 3.2 | 51.58 | 5.60 |
| | | | 1.6 | 46.73** | 6.15** |
| | | | 0 | 26.45 | 0.40 |
| hFEN1-KI | | nCas9-T5-EI | 25 | 64.20 | 3.51 |
| (SEQ ID NO: | | | 12.5 | 62.30** | 1.04** |
| 1487) | | | 6.3 | 55.65* | 2.19* |
| | | | 3.2 | 53.60 | 2.03 |
| | | | 1.6 | 47.53** | 2.76** |
| | | | 0 | 17.70** | 3.60** |

*N = 2;
**N = 3

### Example 28—Templated Editing in Primary Human Hepatocytes in the Presence of Enhancers of Template-Based Editing

This Example evaluated templated editing efficiency in the presence of various combinations of accessory factors (i.e., editing enhancers). The editing accessory factors assessed were Vpx and M97 in combination with human FEN1, human FEN1-KI, or human FEN1-EI.

An mRNA encoding editing accessory factors Vpx (SEQ ID NO: 1501) and M97 (SEQ ID NO: 1497), were delivered into primary human hepatocytes (PHH) in combination with either one of hFEN1 (SEQ ID NO: 1506), hFEN1-KI (SEQ ID NO: 1487), or hFEN1-EI (SEQ ID NO: 1492), and in conjunction with a tgRNA (G032596) targeting VEGFA, and a mRNA encoding either one of nCas9-T5pol(v3) (SEQ ID NO: 1222), nCas9-T5-KI (SEQ ID NO: 1256), or nCas9-T5-EI (SEQ ID NO: 1251).

PHH (Gibco, Lot 8381) were prepared as described in Example 1, and plated on a collagen I coated 96-well plate (StemCell Technologies, Cat. 100-0366) at a density of 33,000 cells/well. LNPs comprising a single RNA species were used for lipofection. Each LNP contained one of an mRNA encoding an editing enhancer (hFEN1, hFEN1-KI, hFEN1-EI, M97, or Vpx); an mRNA encoding an nCas9-polymerase fusion protein (nCas9-T5pol(v3), nCas9-T5-KI, or nCas9-T5-EI); or a tgRNA (G032596). The LNPs were prepared as described in Example 1. The LNPs were made with a molar ratio of 50% Lipid A, 38% cholesterol, 9% DSPC, and 3% PEG2k-DMG, and formulated with a lipid amine to RNA phosphate (N:P) molar ratio of about 6. Cells were incubated at 37° C., 5% CO₂ for 24 hours prior to treatment with LNPs. LNPs were incubated in media containing 10% fetal bovine serum (FBS) at 37° C. for 10 minutes. Post-incubation, the LNPs were added to the PHH as follows. For each well, an LNP comprising a mRNA encoding a nCas9-polymerase fusion (nCas9-T5pol(v3), nCas9-T5-KI, or nCas9-T5-EI) and an LNP comprising the tgRNA were delivered at a total RNA amount (tgRNA+mRNA) of 10 ng, with a mass ratio of 1:2 tgRNA:mRNA. In addition to the tgRNA and mRNA encoding the nCas9-polymerase fusion, an LNP comprising either Vpx or M97 was delivered to the cells in an 8-point 2-fold dilution series starting at 25 ng. For each dilution of Vpx or M97, an LNP comprising a FEN1 variant (hFEN1, hFEN1-KI, or hFEN1-EI) was also delivered to the cells in a 6-point 2-fold dilution series starting at 25 ng. The cells were lysed 72 hours post-treatment for NGS analysis as described in Example 1. Samples were run in duplicates.

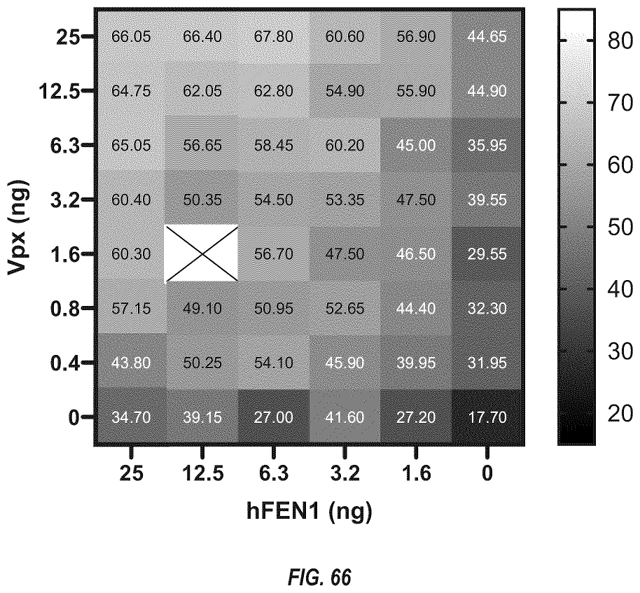

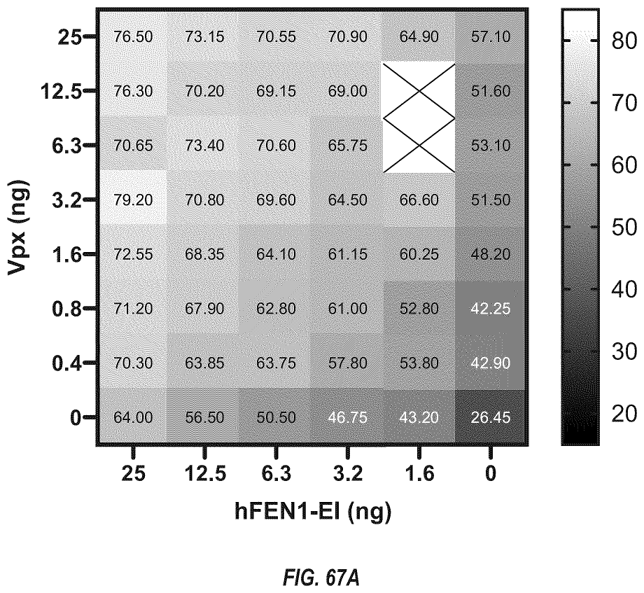

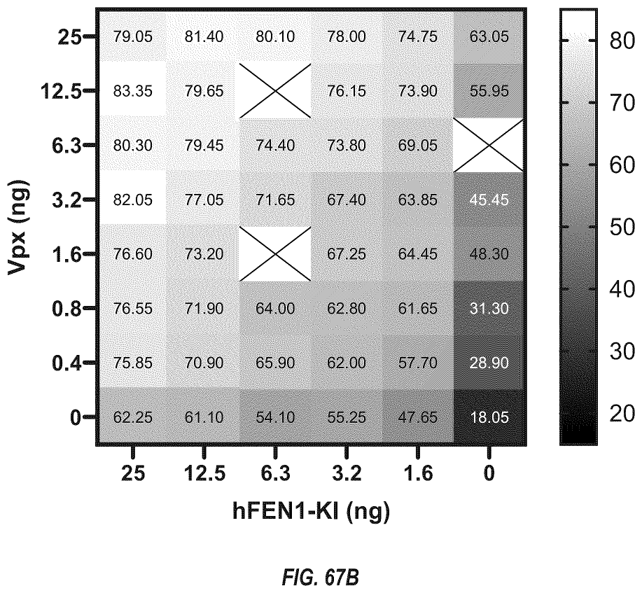

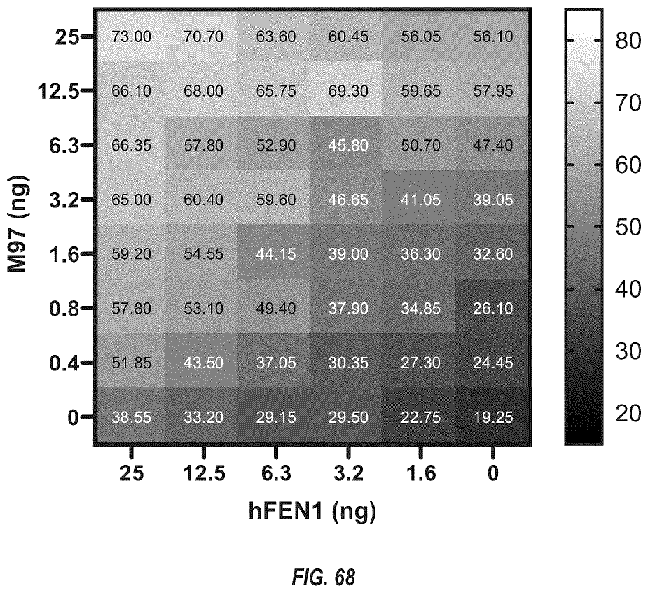

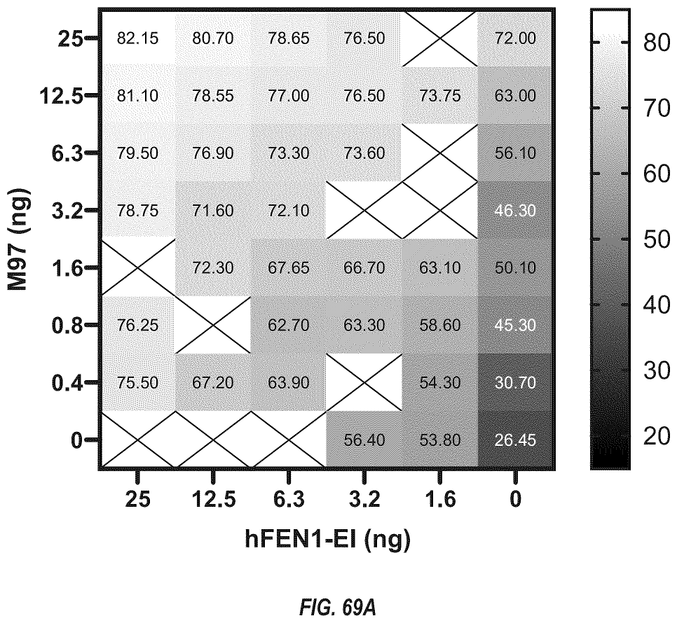

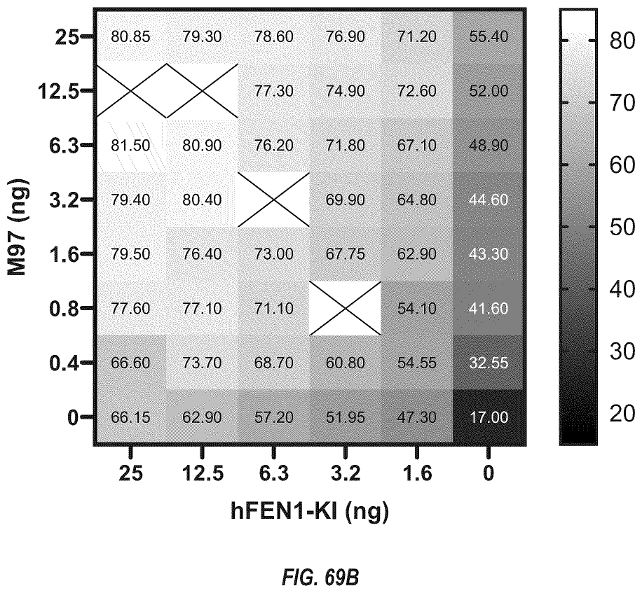

FIG. 66 and Table 72 show editing efficiencies at the VEGFA locus in cells treated with nCas9-T5pol(v3), hFEN1, and Vpx. FIG. 67A and Table 73 show editing efficiencies at the VEGFA locus in cells treated with nCas9-T5-KI, hFEN1-EI, and Vpx. FIG. 67B and Table 74 show editing efficiencies obtained at the VEGFA locus in cells treated with nCas9-T5-EI, hFEN1-KI, and Vpx. FIG. 68 and Table 75 show editing efficiencies at the VEGFA locus in cells treated with nCas9-T5pol(v3), hFEN1, and M97. FIG. 69a and Table 76 show editing efficiencies at the VEGFA locus in cells treated with nCas9-T5-KI, hFEN1-EI, and M97. FIG. 69B and Table 77 show editing efficiencies at the VEGFA locus in cells treated with nCas9-T5-EI, hFEN1-KI, and M97.

TABLE 72
Editing efficiencies at the VEGFA locus in cells transfected with nCas9-
T5pol(v3) in the presence of serial dilutions of hFEN1 and Vpx

| | hFEN1 (ng) | | | | | | | | | | | |
|---|---|---|---|---|---|---|---|---|---|---|---|---|
| Vpx | 25 | | 12.5 | | 6.3 | | 3.2 | | 1.6 | | 0 | |
| (ng) | Mean | SD | Mean | SD | Mean | SD | Mean | SD | Mean | SD | Mean | SD |
| 25 | 66.05 | 6.72 | 66.40 | 0.14 | 67.80 | 0.14 | 60.60 * | N/A | 56.90 | 3.39 | 44.65 | 2.90 |
| 12.5 | 64.75 | 11.81 | 62.05 | 7.14 | 62.80 | 9.05 | 54.90 | 0.28 | 55.90 | 9.33 | 44.90 * | N/A |
| 6.3 | 65.05 | 1.20 | 56.65 | 4.31 | 58.45 | 1.77 | 60.20 | 14.00 | 45.00 | 1.70 | 35.95 | 0.64 |
| 3.2 | 60.40 | 9.33 | 50.35 | 7.14 | 54.50 | 1.98 | 53.35 | 2.47 | 47.50 | 2.26 | 39.55 | 6.01 |
| 1.6 | 60.30 | 1.41 | no | no | 56.70 * | N/A | 47.50 * | N/A | 46.50 * | N/A | 29.55 | 5.30 |
| | | | data | data | | | | | | | | |
| 0.8 | 57.15 | 8.70 | 49.10 * | N/A | 50.95 | 6.86 | 52.65 | 7.28 | 44.40 * | N/A | 32.30 | 0.57 |
| 0.4 | 43.80 * | N/A | 50.25 | 12.94 | 54.10 | 8.63 | 45.90 | 7.07 | 39.95 | 9.12 | 31.95 | 7.57 |
| 0 | 34.70 * | N/A | 39.15 | 10.11 | 27.00 * | N/A | 41.60 | 22.91 | 27.20 * | N/A | 17.70 * | N/A |

* N = 1

TABLE 73
Editing efficiencies at the VEGFA locus in cells transfected with
nCas9-T5-KI in the presence of serial dilutions of hFEN1-EI and Vpx

| | hFEN1-EI (ng) | | | | | | | | | | | |
|---|---|---|---|---|---|---|---|---|---|---|---|---|
| | 25 | | 12.5 | | 6.3 | | 3.2 | | 1.6 | | 0 | |
| Vpx (ng) | Mean | SD | Mean | SD | Mean | SD | Mean | SD | Mean | SD | Mean | SD |
| 25 | 76.50 | 2.26 | 73.15 | 1.34 | 70.55 | 1.48 | 70.90* | N/A | 64.90* | N/A | 57.10* | N/A |
| 12.5 | 76.30* | N/A | 70.20 | 2.83 | 69.15 | 3.75 | 69.00* | N/A | no data | no data | 51.60 | 2.12 |
| 6.3 | 70.65 | 6.01 | 73.40 | 2.83 | 70.60* | N/A | 65.75 | 4.17 | no data | no data | 53.10* | N/A |
| 3.2 | 79.20* | N/A | 70.80* | N/A | 69.60 | 5.52 | 64.50 | 5.66 | 66.60* | N/A | 51.50* | N/A |
| 1.6 | 72.55 | 2.19 | 68.35 | 4.03 | 64.10* | N/A | 61.15 | 0.78 | 60.25 | 1.63 | 48.20 | 2.55 |
| 0.8 | 71.20* | N/A | 67.90* | N/A | 62.80 | 7.92 | 61.00 | 4.38 | 52.80 | 7.64 | 42.25 | 0.64 |
| 0.4 | 70.30* | N/A | 63.85 | 0.78 | 63.75 | 1.91 | 57.80 | 1.13 | 53.80 | 5.66 | 42.90* | N/A |
| 0 | 64.00 | 3.54 | 56.50 | 0.71 | 50.50 | 4.67 | 46.75 | 0.78 | 43.20 | 0.85 | 26.45 | 0.49 |

*N = 1

TABLE 74
Editing efficiencies obtained for at the VEGFA locus in cells transfected
with nCas9-T5-EI in the presence of serial dilutions of hFEN1-KI and Vpx

| | hFEN1-KI (ng) | | | | | | | | | | | |
|---|---|---|---|---|---|---|---|---|---|---|---|---|
| | 25 | | 12.5 | | 6.3 | | 3.2 | | 1.6 | | 0 | |
| Vpx (ng) | Mean | SD | Mean | SD | Mean | SD | Mean | SD | Mean | SD | Mean | SD |
| 25 | 79.05 | 1.63 | 81.40 | 1.98 | 80.10* | N/A | 78.00 | 3.82 | 74.75 | 4.88 | 63.05 | 7.57 |
| 12.5 | 83.35 | 1.91 | 79.65 | 2.47 | no data | no data | 76.15 | 2.90 | 73.90 | 3.54 | 55.95 | 5.16 |
| 6.3 | 80.30 | 2.83 | 79.45 | 0.21 | 74.40 | 0.85 | 73.80 | 0.14 | 69.05 | 1.91 | no data | no data |
| 3.2 | 82.05 | 1.20 | 77.05 | 3.18 | 71.65 | 2.47 | 67.40 | 4.24 | 63.85 | 0.07 | 45.45 | 3.04 |
| 1.6 | 76.60* | N/A | 73.20* | N/A | no data | no data | 67.25 | 5.73 | 64.45 | 0.64 | 48.30* | N/A |
| 0.8 | 76.55 | 1.77 | 71.90* | N/A | 64.00* | N/A | 62.80* | N/A | 61.65 | 4.45 | 31.30* | N/A |
| 0.4 | 75.85 | 1.77 | 70.90 | 5.23 | 65.90 | 2.40 | 62.00* | N/A | 57.70 | 4.24 | 28.90 | 6.79 |
| 0 | 62.25 | 2.76 | 61.10* | N/A | 54.10* | N/A | 55.25 | 1.06 | 47.65 | 3.89 | 18.05 | 5.02 |

*N = 1

TABLE 75
Editing efficiencies at the VEGFA locus in cells transfected with nCas9-T5pol(v3), hFEN1, and M97

| | hFEN1 (ng) | | | | | | | | | | | |
|---|---|---|---|---|---|---|---|---|---|---|---|---|
| | 25 | | 12.5 | | 6.3 | | 3.2 | | 1.6 | | 0 | |
| M97 (ng) | Mean | SD | Mean | SD | Mean | SD | Mean | SD | Mean | SD | Mean | SD |
| 25 | 73.00* | N/A | 70.70* | N/A | 63.60 | 2.55 | 60.45 | 5.87 | 56.05 | 5.87 | 56.10 | 6.08 |
| 12.5 | 66.10* | N/A | 68.00 | 3.54 | 65.75 | 4.88 | 69.30* | N/A | 59.65 | 11.67 | 57.95 | 6.29 |
| 6.3 | 66.35 | 0.64 | 57.80* | N/A | 52.90* | N/A | 45.80* | N/A | 50.70 | 15.56 | 47.40 | 6.65 |
| 3.2 | 65.00 | 5.09 | 60.40* | N/A | 59.60* | N/A | 46.65 | 9.97 | 41.05 | 12.23 | 39.05 | 3.46 |
| 1.6 | 59.20* | N/A | 54.55 | 4.60 | 44.15 | 3.32 | 39.00 | 9.62 | 36.30 | 5.94 | 32.60 | 7.50 |
| 0.8 | 57.80* | N/A | 53.10* | N/A | 49.40* | N/A | 37.90 | 0.14 | 34.85 | 2.62 | 26.10 | 0.57 |
| 0.4 | 51.85 | 1.48 | 43.50 | 3.39 | 37.05 | 4.31 | 30.35 | 6.15 | 27.30 | 6.36 | 24.45 | 3.32 |
| 0 | 38.55 | 1.91 | 33.20 | 0.00 | 29.15 | 0.49 | 29.50 | 6.36 | 22.75 | 0.07 | 19.25 | 3.46 |

*N = 1

TABLE 76
Editing efficiencies at the VEGFA locus in cells transfected
with nCas9-T5-KI in the presence of hFEN1-EI and M97

| | hFEN1-EI (ng) | | | | | | | | | | | |
|---|---|---|---|---|---|---|---|---|---|---|---|---|
| | 25 | | 12.5 | | 6.3 | | 3.2 | | 1.6 | | 0 | |
| M97 (ng) | Mean | SD | Mean | SD | Mean | SD | Mean | SD | Mean | SD | Mean | SD |
| 25 | 82.15 | 0.64 | 80.70 | 3.25 | 78.65 | 2.47 | 76.50* | N/A | no | no | 72.00* | N/A |
| | | | | | | | | | data | data | | |
| 12.5 | 81.10 | 2.26 | 78.55 | 1.91 | 77.00* | N/A | 76.50 | 1.27 | 73.75 | 1.77 | 63.00* | N/A |
| 6.3 | 79.50 | 2.69 | 76.90* | N/A | 73.30* | N/A | 73.60* | N/A | no | no | 56.10* | N/A |
| | | | | | | | | | data | data | | |
| 3.2 | 78.75 | 2.47 | 71.60* | N/A | 72.10 | 1.13 | no | no | no | no | 46.30* | N/A |
| | | | | | | | data | data | data | data | | |
| 1.6 | no | no | 72.30* | N/A | 67.65 | 4.03 | 66.70* | N/A | 63.10* | N/A | 50.10 | 3.82 |
| | data | data | | | | | | | | | | |
| 0.8 | 76.25 | 1.06 | no | no | 62.70* | N/A | 63.30* | N/A | 58.60* | N/A | 45.30* | N/A |
| | | | data | data | | | | | | | | |
| 0.4 | 75.50* | N/A | 67.20* | N/A | 63.90* | N/A | no | no | 54.30* | N/A | 30.70* | N/A |
| | | | | | | | data | data | | | | |
| 0 | no | no | no | no | no | no | 56.40 | 0.57 | 53.80* | N/A | 26.45 | 0.49 |
| | data | data | data | data | data | data | | | | | | |

*N = 1

TABLE 77
Editing efficiencies at the VEGFA locus in cells transfected
with nCas9-T5-EI in the presence hFEN1-KI

| | hFEN1-KI (ng) | | | | | | | | | | | |
|---|---|---|---|---|---|---|---|---|---|---|---|---|
| M97 | 25 | | 12.5 | | 6.3 | | 3.2 | | 1.6 | | 0 | |
| (ng) | Mean | SD | Mean | SD | Mean | SD | Mean | SD | Mean | SD | Mean | SD |
| 25 | 80.85 | 0.35 | 79.30* | N/A | 78.60 | 1.56 | 76.90 | 2.26 | 71.20* | N/A | 55.40 | 7.35 |
| 12.5 | no | no | no | no | 77.30* | N/A | 74.90* | N/A | 72.60* | N/A | 52.00* | N/A |
| | data | data | data | data | | | | | | | | |
| 6.3 | 81.50* | N/A | 80.90* | N/A | 76.20* | N/A | 71.80* | N/A | 67.10* | N/A | 48.90* | N/A |
| 3.2 | 79.40* | N/A | 80.40* | N/A | no | no | 69.90 | 1.27 | 64.80 | 2.83 | 44.60* | N/A |
| | | | | | data | data | | | | | | |
| 1.6 | 79.50* | N/A | 76.40* | N/A | 73.00* | N/A | 67.75 | 3.89 | 62.90* | N/A | 43.30* | N/A |
| 0.8 | 77.60* | N/A | 77.10* | N/A | 71.10* | N/A | no | no | 54.10* | N/A | 41.60* | N/A |
| | | | | | | | data | data | | | | |
| 0.4 | 66.60* | N/A | 73.70* | N/A | 68.70* | N/A | 60.80 | N/A | 54.55 | 0.07 | 32.55 | 1.06 |
| 0 | 66.15 | 3.75 | 62.90 | 0.00 | 57.20* | N/A | 51.95 | 0.64 | 47.30* | N/A | 17.00* | N/A |

*N = 1

### Example 29—Templated Editing in Primary Human Hepatocytes and Huh7 Cells in the Presence of Different Combinations of Accessory Factors

This Example evaluated templated editing efficiency in the presence of various combinations of accessory factors (i.e., editing enhancers). The editing accessory factor Vpx was assessed in combination with FEN1 or MLH1-dn. Different accessory factors were tested in PHH and Huh7 cells to evaluate their effects on editing with polk-nCas9 (SEQ ID NO: 1025) at the VEGFA locus.

PHH (Gibco, Lot 8381) were prepared as described in Example 1, and plated on a collagen I coated 96-well plate (StemCell Technologies, Cat. 100-0366) at a density of 33,000 cells/well. Huh7 cells were prepared as described in Example 1, plated on a collagen I coated 96-well plate (StemCell Technologies, Cat. 100-1056) at a density of 15,000 cells/well, and incubated in a tissue culture incubator at 37° C. and 5% CO₂ atmosphere for 24 hours.

LNPs comprising a single RNA species were used for lipofection. Each LNP contained one of a tgRNA targeting VEGFA (G032595), or an mRNA encoding one of polK-nCas9, FEN1 (SEQ ID NO: 1506), or Vpx (SEQ ID NO: 1501). The LNPs were prepared as described in Example 1, using a molar ratio of 50% Lipid A, 38% cholesterol, 9% DSPC, and 3% PEG2k-DMG, and formulated with a lipid amine to RNA phosphate (N:P) molar ratio of about 6. Cells were incubated at 37° C., 5% CO₂ for 24 hours prior to treatment with LNPs. LNPs were incubated in media containing 10% fetal bovine serum (FBS) at 37° C. for 10 minutes.

In PHH post-incubation, LNPs comprising the mRNA encoding the polK-Cas9 and LNPs comprising the G032595 guide RNA were added at 100 ng total RNA (mRNA+gRNA) in a mRNA: gRNA weight ratio of 2:1. LNPs comprising Vpx were added at a dose of 0.6 ng mRNA, and LNPs comprising FEN1 were also added to the PHH in a 6-point 3-fold dilution series starting at 50 ng mRNA. Control groups consisted of PHH transfected with G032595 and the mRNA encoding polk-nCas9 without Vpx or FEN1.

In huh7 cells post-incubation, LNPs comprising the mRNA encoding the polK-Cas9 and LNPs comprising the G032595 guide RNA were added at 50 ng total RNA (mRNA+gRNA) in a mRNA: gRNA weight ratio of 2:1. LNPs comprising MLH1-dn (SEQ ID NO: 1548) were also added to the huh7 cells in a 6-point 3-fold dilution series starting at 50 ng. Control groups consisted of PHH transfected with G032595 and the mRNA encoding polk-nCas9 without MLH1-dn.

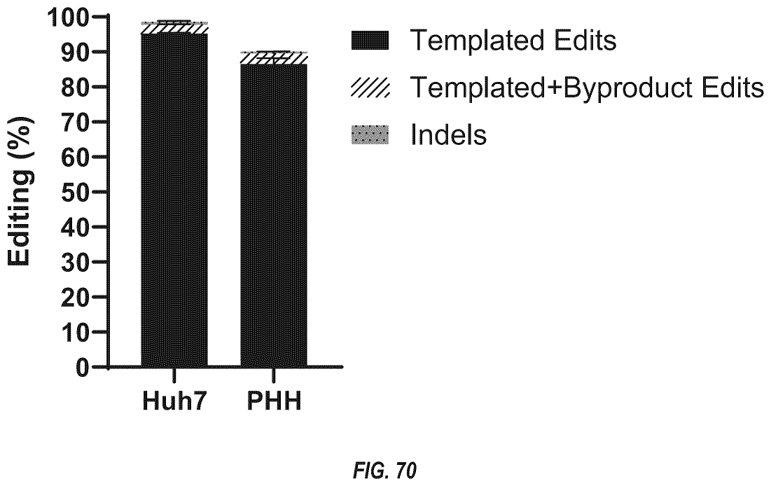

The cells were lysed 72 hours post-treatment for NGS analysis as described in Example 1 with PHRED>10 (data with PHRED>20 varies from the data reported here by less than 1%). Samples were run in duplicate. The different concentrations of editing accessory factors used generated similar levels of editing in each cell type. FIG. 70 and Table 78 show levels of editing in PHH transfected with 0.6 ng Vpx and 1.9 ng FEN1, and in huh7 cells transfected with 50 ng of MLH1-dn.

TABLE 78
Templated editing at the VEGFA locus in cells
treated with Vpx and FEN1 or MLH1-dn

| | Templated Edits | | Templated + Byproduct Edits | | Indels | |
|---|---|---|---|---|---|---|
| | Mean % | SD | Mean % | SD | Mean % | SD |
| Huh7 | 95.2 | 0.3 | 2.5 | 0.1 | 0.9 | 0.3 |
| PHH | 86.5 | 1.8 | 3.0 | 0.7 | 0.7 | 0.0 |

### Example 30—Templated Editing at the VEGFA Locus in Human Bronchial Epithelial Cells

This example assessed templated editing in human bronchial epithelial cells. mRNAs encoding fusion proteins comprising a SpyCas9 nickase (nCas9) moiety and a DNA polymerase moiety were delivered to human bronchial epithelial (16HBE) cells, along with a tgRNA targeting the VEGFA locus (G032595).

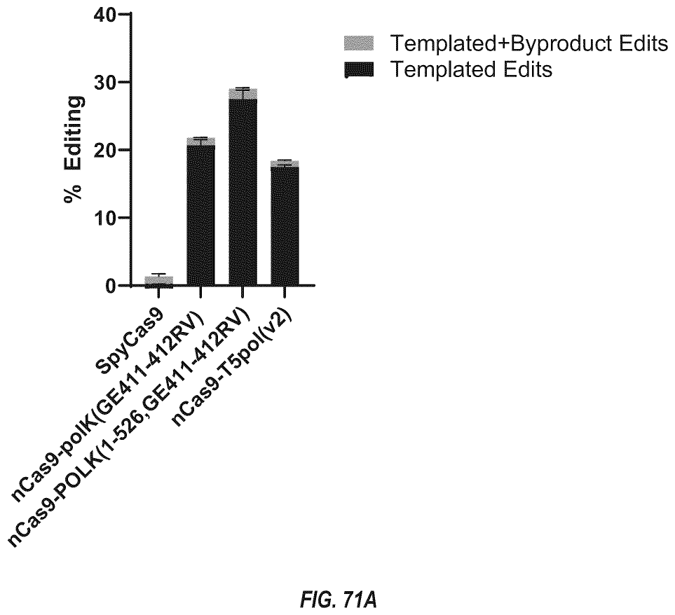

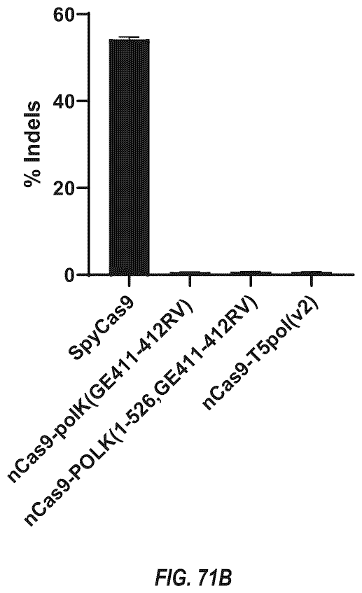

16HBE cells comprising a F508del mutation were prepared using methods known in the art, plated at a density of 15,000 cells/well in a 96-well plate format and incubated at 37° C. and 5% CO₂ atmosphere for 24 hours. Nucleic acid components were delivered using Lipofectamine™ MessengerMAX™ Reagent (Thermo Fisher, Cat. LMRNA001) according to the manufacturer's protocol. 16HBE cells were transfected with G032595, along with an mRNA encoding either a SpyCas9 or a fusion protein corresponding to one of nCas9-polK(GE411-412RV) (SEQ ID NO: 1028), nCas9-POLK(1-526,GE411-412RV) (SEQ ID NO: 1266), and nCas9-T5pol(v2) (SEQ ID NO: 1217), at 200 ng total RNA (mRNA+gRNA) in a tgRNA:mRNA ratio of 1:2 by weight. Samples were run in triplicate. Cells were lysed 3 days post-treatment for NGS analysis as described in Example 1 to measure editing at the VEGFA locus. Mean percent templated editing with standard deviation (SD), and templated editing with byproduct edits, are shown in Table 79 and FIG. 71A. Mean indel percentage with SD is shown in Table 80 and FIG. 71B.

TABLE 79
Editing analysis in 16HBE cells transfected with a
mRNA encoding SpyCas9 or a fusion protein comprising
a SpyCas9 nickase moiety and a polymerase moiety.

| | Templated Edits | | Templated + Byproduct Edits | |
|---|---|---|---|---|
| | Mean % | SD | Mean % | SD |
| SpyCas9 | 0.17 | 0.06 | 1.17 | 0.40 |
| nCas9-polK(GE411- | 20.67 | 0.84 | 1.13 | 0.06 |
| 412RV) | | | | |
| nCas9-POLK(1-526, | 27.47 | 1.36 | 1.57 | 0.15 |
| GE411-412RV) | | | | |
| nCas9-T5pol(v2) | 17.47 | 0.32 | 0.93 | 0.12 |

TABLE 80
Indel analysis in 16HBE cells transfected with a mRNA
encoding SpyCas9 or a fusion protein comprising a
SpyCas9 nickase moiety and a polymerase moiety.

Indels

| | Mean % | SD |
|---|---|---|
| SpyCas9 | 54.23 | 0.55 |
| nCas9-polK(GE411-412RV) | 0.63 | 0.06 |
| nCas9-POLK(1-526, GE411-412RV) | 0.73 | 0.06 |
| nCas9-T5pol(v2) | 0.67 | 0.06 |

### Example 31—Templated Editing at the VEGFA Locus in Huh7 Cells Using MS2-Loop Linked 3′ Extensions with Various Chemical Modifications and Lengths

This Example assessed: (a) the editing efficiency of MS2-loop-linked 3′ extension incorporating deoxynucleotides at various positions 3′ of the DRS/template junction of the tgRNA; (b) the editing efficiency of designs incorporating varying numbers and positions of phosphorothioate (PS) bonds, or 2′-O-methyl modified (2′-Ome) nucleotides within the DRS; and (c) editing efficiency of designs incorporating various numbers and positions of MS2-loops within the MS2-loop-linked 3′ extension.

Indel analysis and editing analysis were performed using guide RNAs (gRNAs) targeting the VEGFA locus. These gRNAs consisted of the same sgRNA (G034491) paired with an MS2-loop-linked 3′ extension comprising a DNA template sequence and a DRS sequence.

Guide RNAs and MS2-loop-linked 3′ extensions as described in Table 81 were delivered to Huh7 cells with either a SpyCas9 encoding mRNA (SEQ ID NO: 1001) to observe indels resulting from SpyCas9 cleavage activity, or an mRNA encoding an nCas9-polymerase fusion protein such as nCas9-T5Pol(D164A,E166A)-MCP (SEQ ID No. 1168) or MCP-PolK-nCas9 (SEQ ID No. 1057) as indicated in Table 81.

Huh7 cells were prepared as described in Example 1, and plated at a density of 15,000 cells/well. After 24 hours of incubation at 37° C., 5% CO₂, cells were transfected using Lipofectamine™ MessengerMAX™ Reagent (Invitrogen, Cat. LMRNA015) according to manufacturer's instructions with 133.3 ng of mRNA, 33.3 ng sgRNA (G034491), and 33.3 ng MS2-loop-linked 3′ extension. Control groups treated with only tgRNA (G032595) or sgRNA (G034491) (i.e., no MS2-loop-linked 3′ extension) were transfected with 133 ng of mRNA and a final concentration of 66.7 ng gRNA. Samples were prepared in duplicate. After transfection and incubation at 37° C., 5% CO₂ for 72 hours, samples were prepared for next-generation sequencing (NGS) and analysis for editing efficiency as described in Example 1.

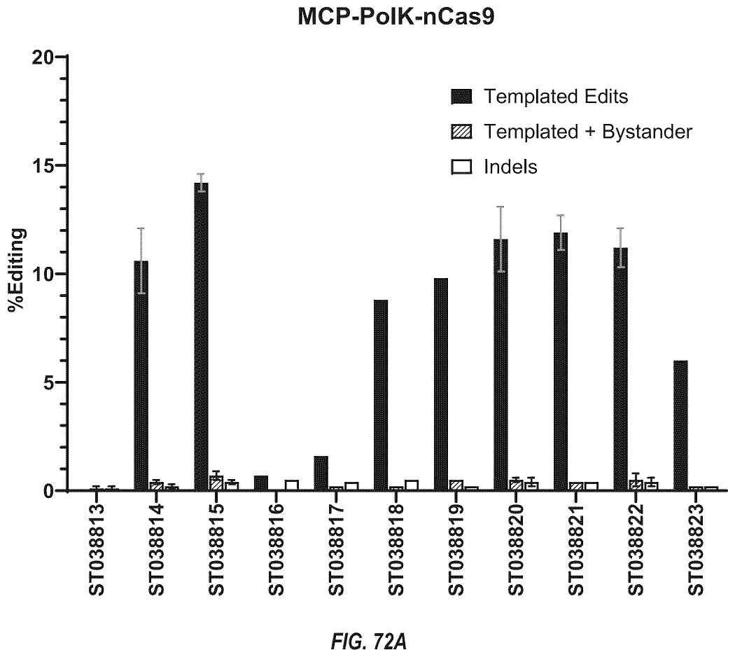

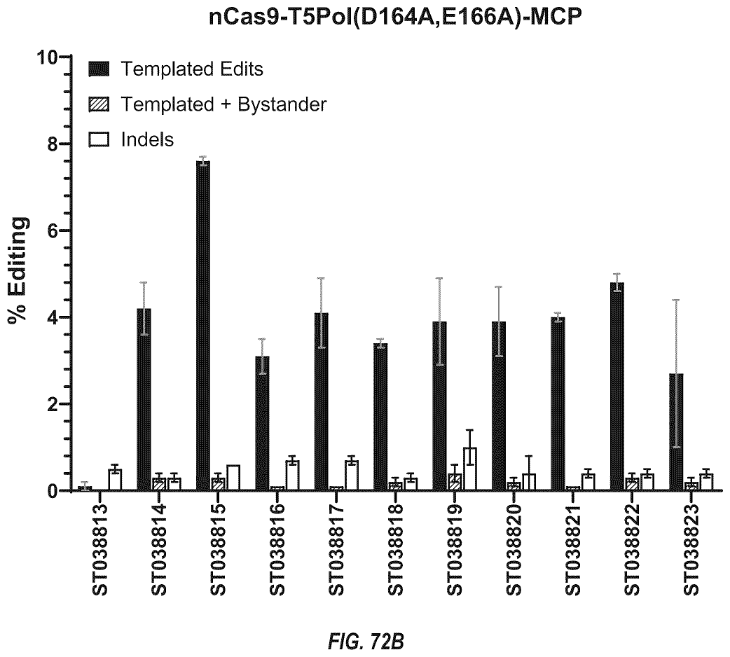

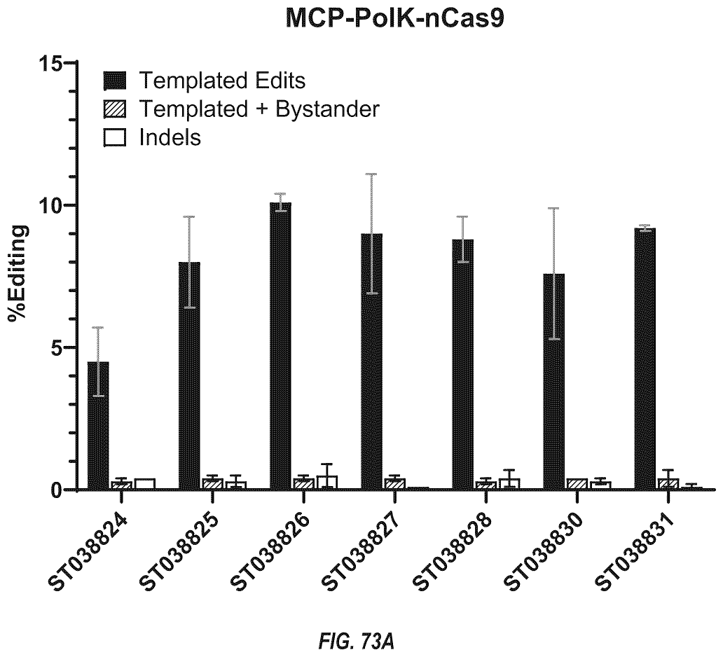

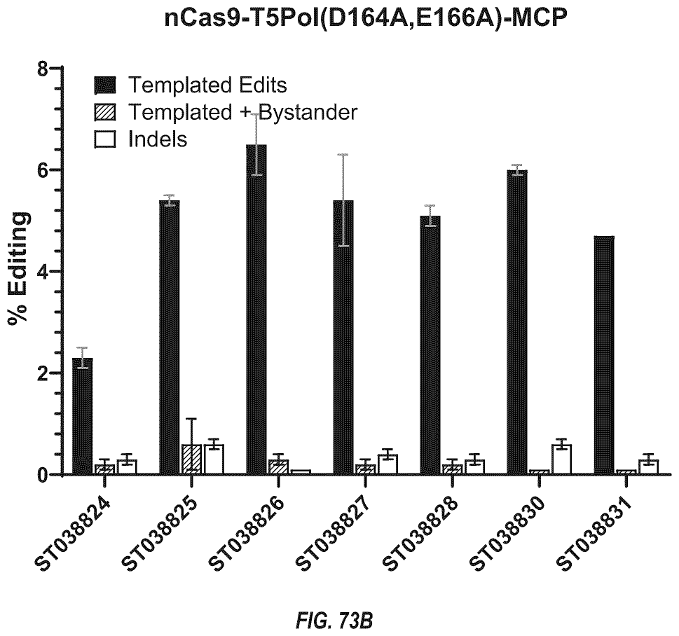

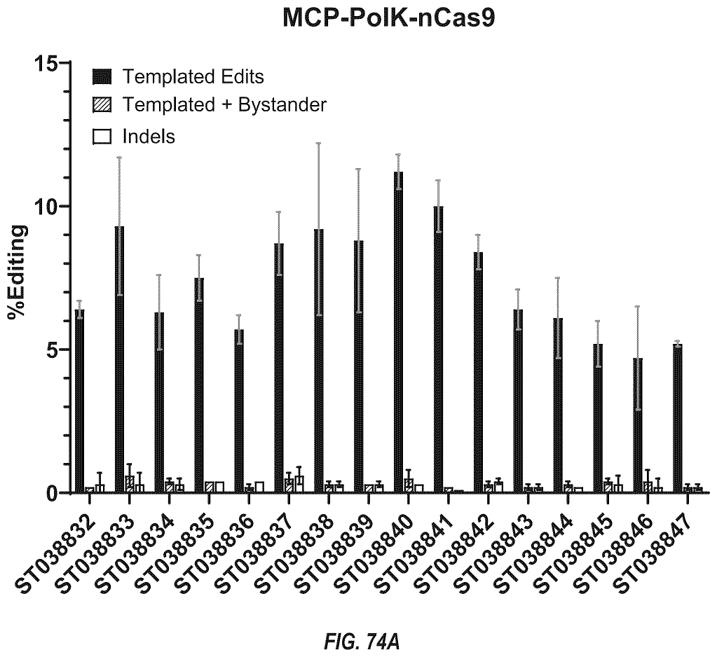

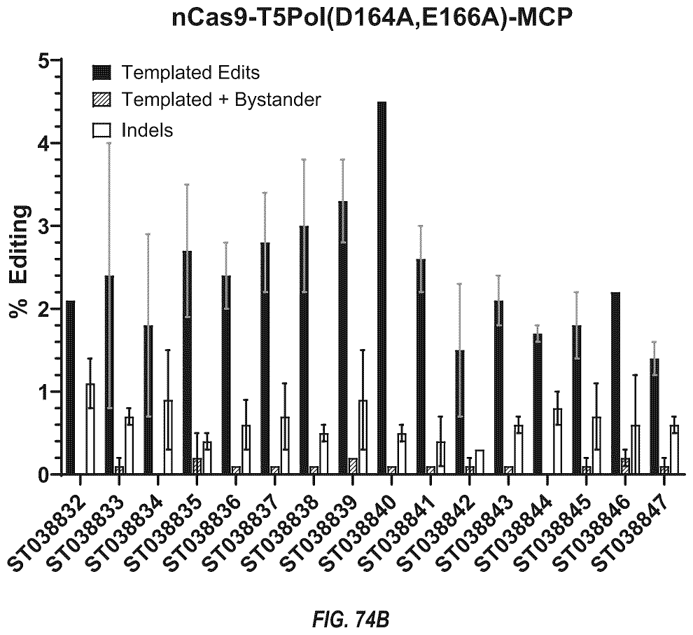

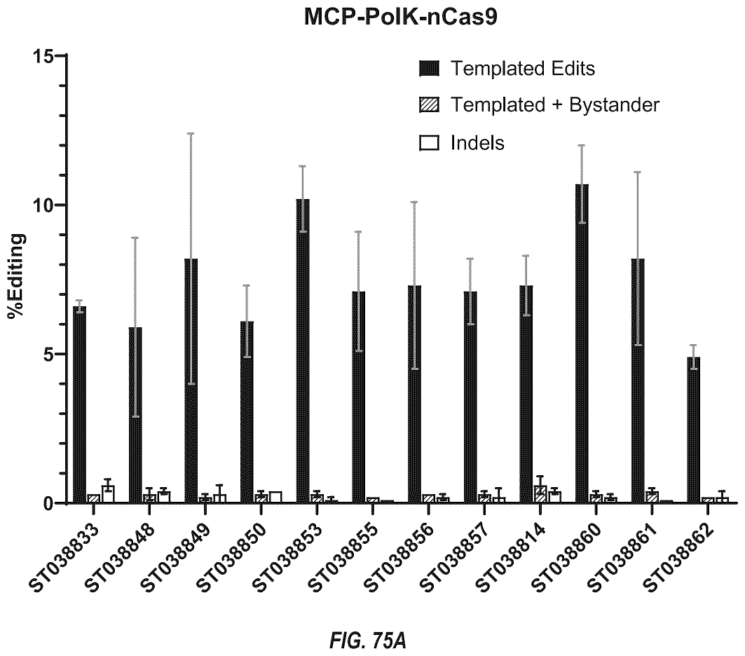

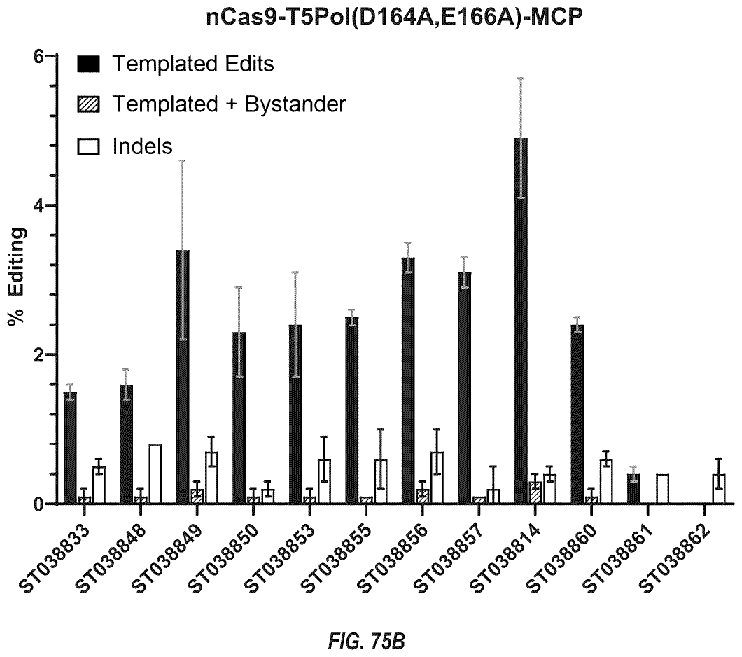

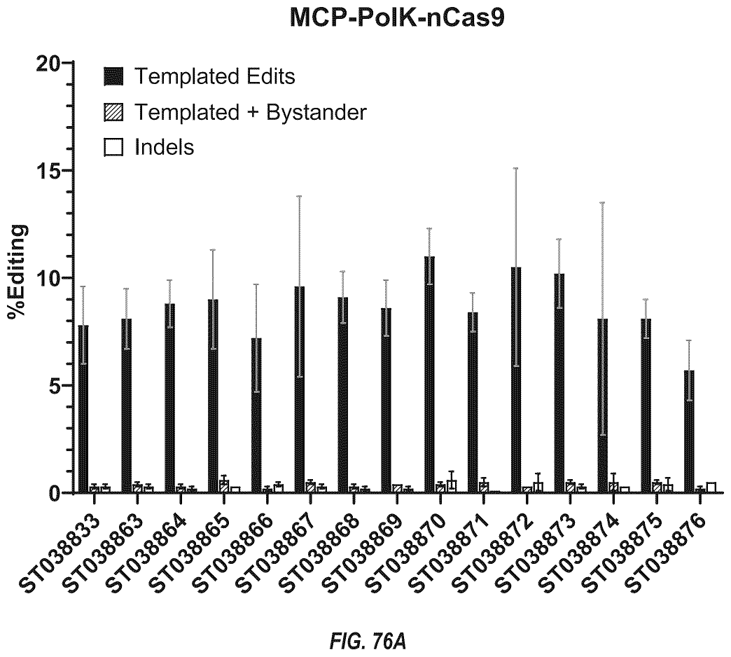

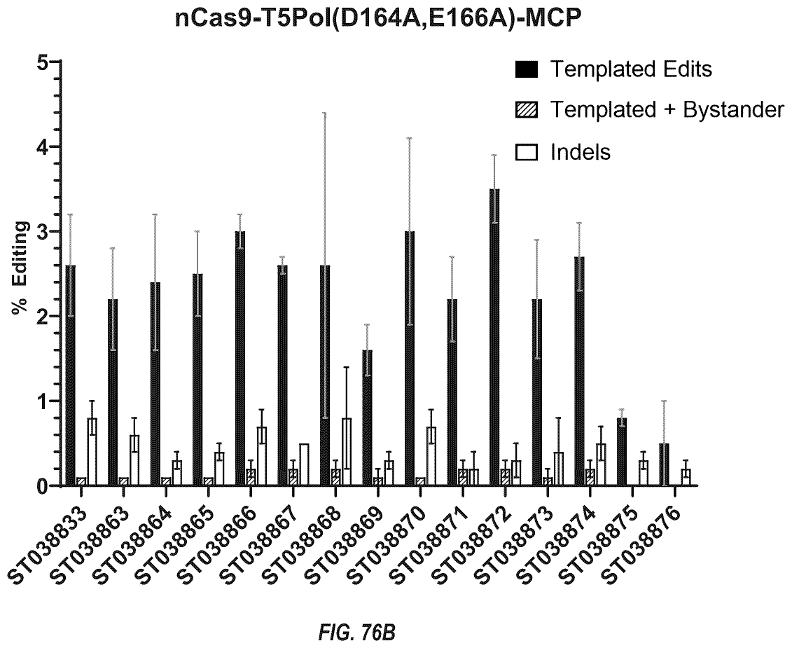

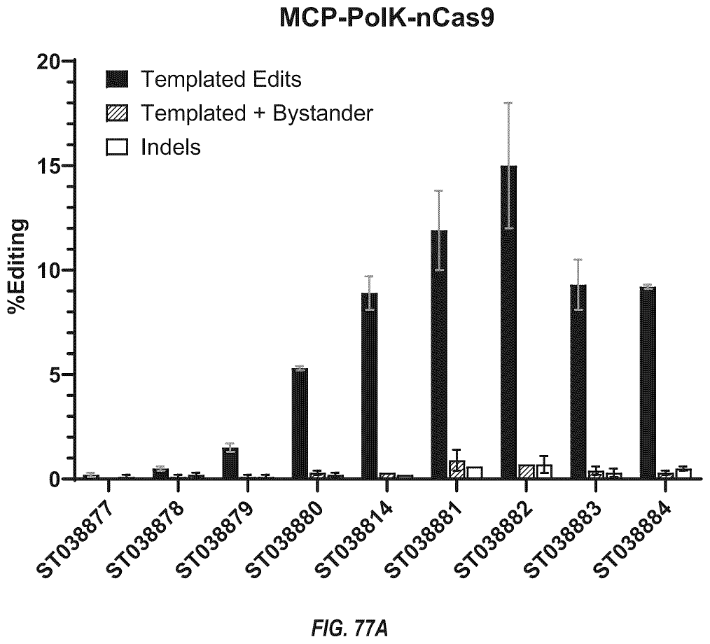

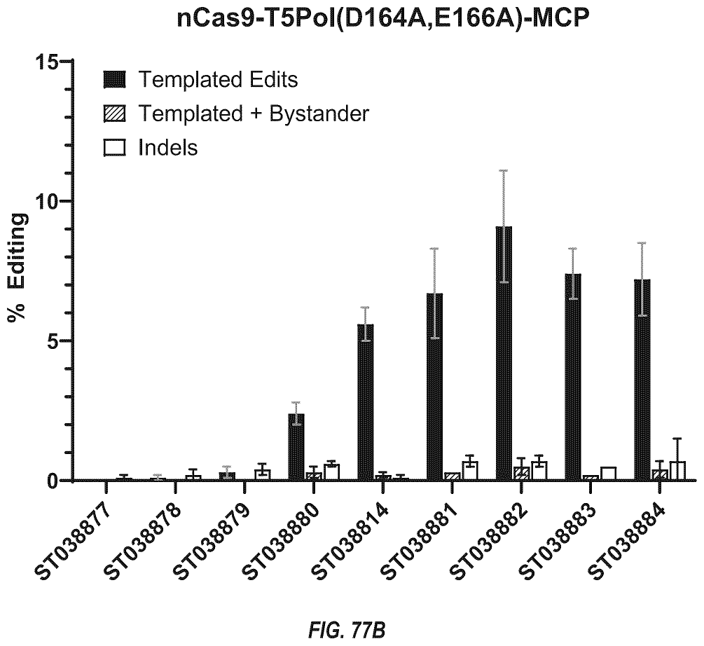

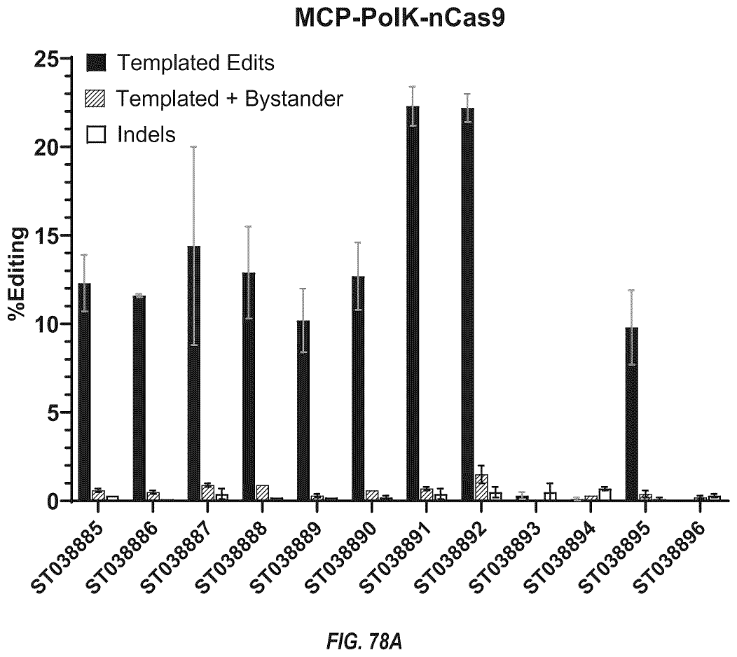

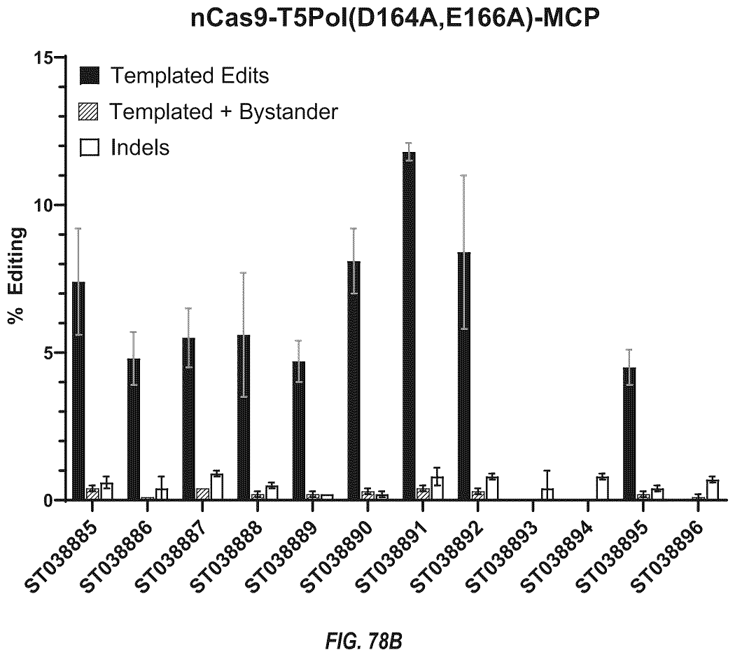

Table 81 shows the editing data at the VEGFA locus using varying guide designs. Three measurements of editing are shown: the “templated edits” the “templated+byproduct edits”, and the ‘indels” corresponding to reads where indels are observed at or near the target site of the tgRNA as described in Example 1. Positions within the DRS and template are defined as described in FIGS. 2A-2B. FIGS. 72A and 72B show editing using guide designs varying the position, number and chemical modifications on or surrounding the MS2-loop feature. FIGS. 73A and B show editing using guide designs varying the number of phosphorothioate bonds (PS). FIGS. 74A and B show editing using guide designs varying the position of phosphorothioate bonds. FIGS. 75A and B show editing using guide designs with varying number of nucleotides with a 2’ O-methyl modification (2′OMe). FIGS. 76A and B show editing using guide designs varying the position of 2′ O-methyl modification. FIGS. 77A and B show editing using guide designs incorporating deoxynuclotides at various positions relative to the template:DRS junction. FIGS. 78A and B show editing using guide designs with varying template lengths, varying DRS lengths, or the incorporation of deoxyuracil at select positions.

Three measurements are shown: the “templated edits”, the “templated+byproduct edits”, and the “indels” as described in Example 1.

TABLE 81
Mean percent of sequencing reads with only templated edits, with templated edits and byproduct edits, and
with indels at the predicted nicking site of the sgRNA for various MS2-loop-linked 3′ extension designs.

| | | | | | | | | | | | nCas9-T5Pol(D164A, E166A)- | | | | | | | | |
|---|---|---|---|---|---|---|---|---|---|---|---|---|---|---|---|---|---|---|---|
| | Description of | MCP-PolK-nCas9 (SEQ ID NO: 1057) | | | | | | | | | MCP (SEQ ID NO: 1168) | | | | | | | | |
| MS2-loop- | modification in the | | | | Templated + | | | | | | Templated | | | Templated + | | | | | |
| linked 3′ | MS2-loop-linked 3′ | Templated Edits | | | Byproduct | | | Indels | | | Edits | | | Byproduct | | | Indels | | |
| extension ID | extension | Mean | SD | n | Mean | SD | n | Mean | SD | n | Mean | SD | n | Mean | SD | n | Mean | SD | n |
| ST038813 (SEQ | rNTP | 0.0 | 0.0 | 2 | 0.1 | 0.1 | 2 | 0.1 | 0.1 | 2 | 0.1 | 0.1 | 2 | 0.0 | 0.0 | 2 | 0.5 | 0.1 | 2 |
| ID NO: 799) | | | | | | | | | | | | | | | | | | | |
| ST038814 (SEQ | 1 MS2 5′ | 10.6 | 1.5 | 2 | 0.4 | 0.1 | 2 | 0.2 | 0.1 | 2 | 4.2 | 0.6 | 2 | 0.3 | 0.1 | 2 | 0.3 | 0.1 | 2 |
| ID NO: 487) | | | | | | | | | | | | | | | | | | | |
| ST038815 (SEQ | 2 MS2 5′ | 14.2 | 0.4 | 2 | 0.7 | 0.2 | 2 | 0.4 | 0.1 | 2 | 7.6 | 0.1 | 2 | 0.3 | 0.1 | 2 | 0.6 | 0.0 | 2 |
| ID NO: 801) | | | | | | | | | | | | | | | | | | | |
| ST038816 (SEQ | 1 MS2 3′ | 0.7 | 0.0 | 1 | 0.0 | 0.0 | 1 | 0.5 | 0.0 | 1 | 3.1 | 0.4 | 2 | 0.1 | 0.0 | 2 | 0.7 | 0.1 | 2 |
| ID NO: 802) | | | | | | | | | | | | | | | | | | | |
| ST038817 (SEQ | 2 MS2 3′ | 1.6 | 0.0 | 1 | 0.2 | 0.0 | 1 | 0.4 | 0.0 | 1 | 4.1 | 0.8 | 2 | 0.1 | 0.0 | 2 | 0.7 | 0.1 | 2 |
| ID NO: 803) | | | | | | | | | | | | | | | | | | | |
| ST038818 (SEQ | 2 MS2 5′ and 3′ | 8.8 | 0.0 | 1 | 0.2 | 0.0 | 1 | 0.5 | 0.0 | 1 | 3.4 | 0.1 | 2 | 0.2 | 0.1 | 2 | 0.3 | 0.1 | 2 |
| ID NO: 804) | | | | | | | | | | | | | | | | | | | |
| ST038819 (SEQ | End-mod-MS2 | 9.8 | 0.0 | 1 | 0.5 | 0.0 | 1 | 0.2 | 0.0 | 1 | 3.9 | 1.0 | 2 | 0.4 | 0.2 | 2 | 1.0 | 0.4 | 2 |
| ID NO: 805) | | | | | | | | | | | | | | | | | | | |
| ST038820 (SEQ | End-mod-MS2- | 11.6 | 1.5 | 2 | 0.5 | 0.1 | 2 | 0.4 | 0.2 | 2 | 3.9 | 0.8 | 2 | 0.2 | 0.1 | 2 | 0.4 | 0.4 | 2 |
| ID NO: 806) | No_5′ps | | | | | | | | | | | | | | | | | | |
| ST038821 (SEQ | End-mod-MS2- | 11.9 | 0.8 | 2 | 0.4 | 0.0 | 2 | 0.4 | 0.0 | 2 | 4.0 | 0.1 | 2 | 0.1 | 0.0 | 2 | 0.4 | 0.1 | 2 |
| ID NO: 807) | No_ps | | | | | | | | | | | | | | | | | | |
| ST038822 (SEQ | Fully 2′-Methylated | 11.2 | 0.9 | 2 | 0.5 | 0.3 | 2 | 0.4 | 0.2 | 2 | 4.8 | 0.2 | 2 | 0.3 | 0.1 | 2 | 0.4 | 0.1 | 2 |
| ID NO: 808) | MS2 | | | | | | | | | | | | | | | | | | |
| ST038823 (SEQ | MS2 complement | 6.0 | 0.0 | 1 | 0.2 | 0.0 | 1 | 0.2 | 0.0 | 1 | 2.7 | 1.7 | 2 | 0.2 | 0.1 | 2 | 0.4 | 0.1 | 2 |
| ID NO: 809) | | | | | | | | | | | | | | | | | | | |
| ST038824 (SEQ | PS D17 | 4.5 | 1.2 | 2 | 0.3 | 0.1 | 2 | 0.4 | 0.0 | 2 | 2.3 | 0.2 | 2 | 0.2 | 0.1 | 2 | 0.3 | 0.1 | 2 |
| ID NO: 810) | | | | | | | | | | | | | | | | | | | |
| ST038825 (SEQ | PS D16-D17 | 8.0 | 1.6 | 2 | 0.4 | 0.1 | 2 | 0.3 | 0.2 | 2 | 5.4 | 0.1 | 2 | 0.6 | 0.5 | 2 | 0.6 | 0.1 | 2 |
| ID NO: 811) | | | | | | | | | | | | | | | | | | | |
| ST038826 (SEQ | PS D15-D17 | 10.1 | 0.3 | 2 | 0.4 | 0.1 | 2 | 0.5 | 0.4 | 2 | 6.5 | 0.6 | 2 | 0.3 | 0.1 | 2 | 0.1 | 0.0 | 2 |
| ID NO: 812) | | | | | | | | | | | | | | | | | | | |
| ST038827 (SEQ | PS D14-D17 | 9.0 | 2.1 | 2 | 0.4 | 0.1 | 2 | 0.1 | 0.0 | 2 | 5.4 | 0.9 | 2 | 0.2 | 0.1 | 2 | 0.4 | 0.1 | 2 |
| ID NO: 813) | | | | | | | | | | | | | | | | | | | |
| ST038828 (SEQ | PS D13-D17 | 8.8 | 0.8 | 2 | 0.3 | 0.1 | 2 | 0.4 | 0.3 | 2 | 5.1 | 0.2 | 2 | 0.2 | 0.1 | 2 | 0.3 | 0.1 | 2 |
| ID NO: 814) | | | | | | | | | | | | | | | | | | | |
| ST038830 (SEQ | PS D18-D19 | 7.6 | 2.3 | 2 | 0.4 | 0.0 | 2 | 0.3 | 0.1 | 2 | 6.0 | 0.1 | 2 | 0.1 | 0.0 | 2 | 0.6 | 0.1 | 2 |
| ID NO: 816) | | | | | | | | | | | | | | | | | | | |
| ST038831 (SEQ | PS D18-D20 | 9.2 | 0.1 | 2 | 0.4 | 0.3 | 2 | 0.1 | 0.1 | 2 | 4.7 | 0.0 | 2 | 0.1 | 0.0 | 2 | 0.3 | 0.1 | 2 |
| ID NO: 817) | | | | | | | | | | | | | | | | | | | |
| ST038832 (SEQ | No PS in DRS | 6.4 | 0.3 | 2 | 0.2 | 0.0 | 2 | 0.3 | 0.4 | 2 | 2.1 | 0.0 | 2 | 0.0 | 0.0 | 2 | 1.1 | 0.3 | 2 |
| ID NO: 818) | | | | | | | | | | | | | | | | | | | |
| ST038833 (SEQ | PS D17 | 9.3 | 2.4 | 2 | 0.6 | 0.4 | 2 | 0.3 | 0.4 | 2 | 2.4 | 1.6 | 2 | 0.1 | 0.1 | 2 | 0.7 | 0.1 | 2 |
| ID NO: 819) | | | | | | | | | | | | | | | | | | | |
| ST038834 (SEQ | PS D16 & D17 | 6.3 | 1.3 | 2 | 0.4 | 0.1 | 2 | 0.3 | 0.2 | 2 | 1.8 | 1.1 | 2 | 0.0 | 0.0 | 2 | 0.9 | 0.6 | 2 |
| ID NO: 820) | | | | | | | | | | | | | | | | | | | |
| ST038835 (SEQ | PS D15& D17 | 7.5 | 0.8 | 2 | 0.4 | 0.0 | 2 | 0.4 | 0.0 | 2 | 2.7 | 0.8 | 2 | 0.2 | 0.3 | 2 | 0.4 | 0.1 | 2 |
| ID NO: 821) | | | | | | | | | | | | | | | | | | | |
| ST038836 (SEQ | PS D14& D17 | 5.7 | 0.5 | 2 | 0.2 | 0.1 | 2 | 0.4 | 0.0 | 2 | 2.4 | 0.4 | 2 | 0.1 | 0.0 | 2 | 0.6 | 0.3 | 2 |
| ID NO: 822) | | | | | | | | | | | | | | | | | | | |
| ST038837 (SEQ | PS D13& D17 | 8.7 | 1.1 | 2 | 0.5 | 0.2 | 2 | 0.6 | 0.3 | 2 | 2.8 | 0.6 | 2 | 0.1 | 0.0 | 2 | 0.7 | 0.4 | 2 |
| ID NO: 823) | | | | | | | | | | | | | | | | | | | |
| ST038838 (SEQ | PS D12& D17 | 9.2 | 3.0 | 2 | 0.3 | 0.1 | 2 | 0.3 | 0.1 | 2 | 3.0 | 0.8 | 2 | 0.1 | 0.0 | 2 | 0.5 | 0.1 | 2 |
| ID NO: 824) | | | | | | | | | | | | | | | | | | | |
| ST038839 (SEQ | PS D11& D17 | 8.8 | 2.5 | 2 | 0.3 | 0.0 | 2 | 0.3 | 0.1 | 2 | 3.3 | 0.5 | 2 | 0.2 | 0.0 | 2 | 0.9 | 0.6 | 2 |
| ID NO: 825) | | | | | | | | | | | | | | | | | | | |
| ST038840 (SEQ | PS D10& D17 | 11.2 | 0.6 | 2 | 0.5 | 0.3 | 2 | 0.3 | 0.0 | 2 | 4.5 | 0.0 | 2 | 0.1 | 0.0 | 2 | 0.5 | 0.1 | 2 |
| ID NO: 826) | | | | | | | | | | | | | | | | | | | |
| ST038841 (SEQ | PS D9& D17 | 10.0 | 0.9 | 2 | 0.2 | 0.0 | 2 | 0.1 | 0.0 | 2 | 2.6 | 0.4 | 2 | 0.1 | 0.0 | 2 | 0.4 | 0.3 | 2 |
| ID NO: 827) | | | | | | | | | | | | | | | | | | | |
| ST038842 (SEQ | PS D8& D17 | 8.4 | 0.6 | 2 | 0.3 | 0.1 | 2 | 0.4 | 0.1 | 2 | 1.5 | 0.8 | 2 | 0.1 | 0.1 | 2 | 0.3 | 0.0 | 2 |
| ID NO: 828) | | | | | | | | | | | | | | | | | | | |
| ST038843 (SEQ | PS D7& D17 | 6.4 | 0.7 | 2 | 0.2 | 0.1 | 2 | 0.2 | 0.1 | 2 | 2.1 | 0.3 | 2 | 0.1 | 0.0 | 2 | 0.6 | 0.1 | 2 |
| ID NO: 829) | | | | | | | | | | | | | | | | | | | |
| ST038844 (SEQ | PS D6& D17 | 6.1 | 1.4 | 2 | 0.3 | 0.1 | 2 | 0.2 | 0.0 | 2 | 1.7 | 0.1 | 2 | 0.0 | 0.0 | 2 | 0.8 | 0.2 | 2 |
| ID NO: 830) | | | | | | | | | | | | | | | | | | | |
| ST038845 (SEQ | PS D5& D17 | 5.2 | 0.8 | 2 | 0.4 | 0.1 | 2 | 0.3 | 0.3 | 2 | 1.8 | 0.4 | 2 | 0.1 | 0.1 | 2 | 0.7 | 0.4 | 2 |
| ID NO: 831) | | | | | | | | | | | | | | | | | | | |
| ST038846 (SEQ | PS D4& D17 | 4.7 | 1.8 | 2 | 0.4 | 0.4 | 2 | 0.2 | 0.3 | 2 | 2.2 | 0.0 | 2 | 0.2 | 0.1 | 2 | 0.6 | 0.6 | 2 |
| ID NO: 832) | | | | | | | | | | | | | | | | | | | |
| ST038847 (SEQ | PS D3& D17 | 5.2 | 0.1 | 2 | 0.2 | 0.1 | 2 | 0.2 | 0.1 | 2 | 1.4 | 0.2 | 2 | 0.1 | 0.1 | 2 | 0.6 | 0.1 | 2 |
| ID NO: 833) | | | | | | | | | | | | | | | | | | | |
| ST038833 (SEQ | 2′OMe D17 | 6.6 | 0.2 | 2 | 0.3 | 0.0 | 2 | 0.6 | 0.2 | 2 | 1.5 | 0.1 | 2 | 0.1 | 0.1 | 2 | 0.5 | 0.1 | 2 |
| ID NO: 834) | | | | | | | | | | | | | | | | | | | |
| ST038848 (SEQ | 2′ OMe D16-D17 | 5.9 | 3.0 | 2 | 0.3 | 0.2 | 2 | 0.4 | 0.1 | 2 | 1.6 | 0.2 | 2 | 0.1 | 0.1 | 2 | 0.8 | 0.0 | 2 |
| ID NO: 835) | | | | | | | | | | | | | | | | | | | |
| ST038849 (SEQ | 2′ OMe D15-D17 | 8.2 | 4.2 | 2 | 0.2 | 0.1 | 2 | 0.3 | 0.3 | 2 | 3.4 | 1.2 | 2 | 0.2 | 0.1 | 2 | 0.7 | 0.2 | 2 |
| ID NO: 836) | | | | | | | | | | | | | | | | | | | |
| ST038850 (SEQ | 2′ OMe D14-D17 | 6.1 | 1.2 | 2 | 0.3 | 0.1 | 2 | 0.4 | 0.0 | 2 | 2.3 | 0.6 | 2 | 0.1 | 0.1 | 2 | 0.2 | 0.1 | 2 |
| ID NO: 837) | | | | | | | | | | | | | | | | | | | |
| ST038853 (SEQ | 2′ OMe D11-D17 | 10.2 | 1.1 | 2 | 0.3 | 0.1 | 2 | 0.1 | 0.1 | 2 | 2.4 | 0.7 | 2 | 0.1 | 0.1 | 2 | 0.6 | 0.3 | 2 |
| ID NO: 840) | | | | | | | | | | | | | | | | | | | |
| ST038855 (SEQ | 2′ OMe D9-D17 | 7.1 | 2.0 | 2 | 0.2 | 0.0 | 2 | 0.1 | 0.0 | 2 | 2.5 | 0.1 | 2 | 0.1 | 0.0 | 2 | 0.6 | 0.4 | 2 |
| ID NO: 842) | | | | | | | | | | | | | | | | | | | |
| ST038856 (SEQ | 2′ OMe D8-D17 | 7.3 | 2.8 | 2 | 0.3 | 0.0 | 2 | 0.2 | 0.1 | 2 | 3.3 | 0.2 | 2 | 0.2 | 0.1 | 2 | 0.7 | 0.3 | 2 |
| ID NO: 843) | | | | | | | | | | | | | | | | | | | |
| ST038857 (SEQ | 2′ OMe D7-D17 | 7.1 | 1.1 | 2 | 0.3 | 0.1 | 2 | 0.2 | 0.3 | 2 | 3.1 | 0.2 | 2 | 0.1 | 0.0 | 2 | 0.2 | 0.3 | 2 |
| ID NO: 844) | | | | | | | | | | | | | | | | | | | |
| ST038814 (SEQ | 2′ OMe D6-D17 | 7.3 | 1.0 | 2 | 0.6 | 0.3 | 2 | 0.4 | 0.1 | 2 | 4.9 | 0.8 | 2 | 0.3 | 0.1 | 2 | 0.4 | 0.1 | 2 |
| ID NO: 487) | | | | | | | | | | | | | | | | | | | |
| ST038860 (SEQ | 2′ OMe D3-D17 | 10.7 | 1.3 | 2 | 0.3 | 0.1 | 2 | 0.2 | 0.1 | 2 | 2.4 | 0.1 | 2 | 0.1 | 0.1 | 2 | 0.6 | 0.1 | 2 |
| ID NO: 848) | | | | | | | | | | | | | | | | | | | |
| ST038861 (SEQ | 2′ OMe D2-D17 | 8.2 | 2.9 | 2 | 0.4 | 0.1 | 2 | 0.1 | 0.0 | 2 | 0.4 | 0.1 | 2 | 0.0 | 0.0 | 2 | 0.4 | 0.0 | 2 |
| ID NO: 849) | | | | | | | | | | | | | | | | | | | |
| ST038862 (SEQ | 2′ OMe D1-D17 | 4.9 | 0.4 | 2 | 0.2 | 0.0 | 2 | 0.2 | 0.2 | 2 | 0.0 | 0.0 | 2 | 0.0 | 0.0 | 2 | 0.4 | 0.2 | 2 |
| ID NO: 850) | | | | | | | | | | | | | | | | | | | |
| ST038833 (SEQ | 2′ OMe D17 | 7.8 | 1.8 | 2 | 0.3 | 0.1 | 2 | 0.3 | 0.1 | 2 | 2.6 | 0.6 | 2 | 0.1 | 0.0 | 2 | 0.8 | 0.2 | 2 |
| ID NO: 819) | | | | | | | | | | | | | | | | | | | |
| ST038863 (SEQ | 2′ OMe D15 & D17 | 8.1 | 1.4 | 2 | 0.4 | 0.1 | 2 | 0.3 | 0.1 | 2 | 2.2 | 0.6 | 2 | 0.1 | 0.0 | 2 | 0.6 | 0.2 | 2 |
| ID NO: 852) | | | | | | | | | | | | | | | | | | | |
| ST038864 (SEQ | 2′ OMe D14 & D17 | 8.8 | 1.1 | 2 | 0.3 | 0.1 | 2 | 0.2 | 0.1 | 2 | 2.4 | 0.8 | 2 | 0.1 | 0.0 | 2 | 0.3 | 0.1 | 2 |
| ID NO: 853) | | | | | | | | | | | | | | | | | | | |
| ST038865 (SEQ | 2′ OMe D13 & D17 | 9.0 | 2.3 | 2 | 0.6 | 0.2 | 2 | 0.3 | 0.0 | 2 | 2.5 | 0.5 | 2 | 0.1 | 0.0 | 2 | 0.4 | 0.1 | 2 |
| ID NO: 854) | | | | | | | | | | | | | | | | | | | |
| ST038866 (SEQ | 2′ OMe D12 & D17 | 7.2 | 2.5 | 2 | 0.2 | 0.1 | 2 | 0.4 | 0.1 | 2 | 3.0 | 0.2 | 2 | 0.2 | 0.1 | 2 | 0.7 | 0.2 | 2 |
| ID NO: 855) | | | | | | | | | | | | | | | | | | | |
| ST038867 (SEQ | 2′ OMe D11 & D17 | 9.6 | 4.2 | 2 | 0.5 | 0.1 | 2 | 0.3 | 0.1 | 2. | 2.6 | 0.1 | 2 | 0.2 | 0.1 | 2 | 0.5 | 0.0 | 2 |
| ID NO: 856) | | | | | | | | | | | | | | | | | | | |
| ST038868 (SEQ | 2′ OMe D10 & D17 | 9.1 | 1.2 | 2 | 0.3 | 0.1 | 2 | 0.2 | 0.1 | 2 | 2.6 | 1.8 | 2 | 0.2 | 0.1 | 2 | 0.8 | 0.6 | 2 |
| ID NO: 857) | | | | | | | | | | | | | | | | | | | |
| ST038869 (SEQ | 2′ OMe D9 & D17 | 8.6 | 1.3 | 2 | 0.4 | 0.0 | 2 | 0.2 | 0.1 | 2 | 1.6 | 0.3 | 2 | 0.1 | 0.1 | 2 | 0.3 | 0.1 | 2 |
| ID NO: 858) | | | | | | | | | | | | | | | | | | | |
| ST038870 (SEQ | 2′ OMe D8 & D17 | 11.0 | 1.3 | 2 | 0.4 | 0.1 | 2 | 0.6 | 0.4 | 2 | 3.0 | 1.1 | 2 | 0.1 | 0.0 | 2 | 0.7 | 0.2 | 2 |
| ID NO: 859) | | | | | | | | | | | | | | | | | | | |
| ST038871 (SEQ | 2′ OMe D7 & D17 | 8.4 | 0.9 | 2 | 0.5 | 0.2 | 2 | 0.1 | 0.0 | 2 | 2.2 | 0.5 | 2 | 0.2 | 0.1 | 2 | 0.2 | 0.2 | 2 |
| ID NO: 860) | | | | | | | | | | | | | | | | | | | |
| ST038872 (SEQ | 2′ OMe D6 & D17 | 10.5 | 4.6 | 2 | 0.3 | 0.0 | 2 | 0.5 | 0.4 | 2 | 3.5 | 0.4 | 2 | 0.2 | 0.1 | 2 | 0.3 | 0.2 | 2 |
| ID NO: 861) | | | | | | | | | | | | | | | | | | | |
| ST038873 (SEQ | 2′ OMe D5 & D17 | 10.2 | 1.6 | 2 | 0.5 | 0.1 | 2 | 0.3 | 0.1 | 2 | 2.2 | 0.7 | 2 | 0.1 | 0.1 | 2 | 0.4 | 0.4 | 2 |
| ID NO: 862) | | | | | | | | | | | | | | | | | | | |
| ST038874 (SEQ | 2′ OMe D4 & D17 | 8.1 | 5.4 | 2. | 0.5 | 0.4 | 2 | 0.3 | 0.0 | 2 | 2.7 | 0.4 | 2 | 0.2 | 0.1 | 2 | 0.5 | 0.2 | 2 |
| ID NO: 863) | | | | | | | | | | | | | | | | | | | |
| ST038875 (SEQ | 2′ OMe D3 & D17 | 8.1 | 0.9 | 2 | 0.5 | 0.1 | 2 | 0.4 | 0.3 | 2 | 0.8 | 0.1 | 2 | 0.0 | 0.0 | 2 | 0.3 | 0.1 | 2 |
| ID NO: 864) | | | | | | | | | | | | | | | | | | | |
| ST038876 (SEQ | 2′ OMe D2 & D17 | 5.7 | 1.4 | 2 | 0.2 | 0.1 | 2 | 0.5 | 0.0 | 2 | 0.5 | 0.5 | 2 | 0.0 | 0.0 | 2 | 0.2 | 0.1 | 2 |
| ID NO: 865) | | | | | | | | | | | | | | | | | | | |
| ST038877 (SEQ | DNA::RNA T4-T3 | 0.2 | 0.1 | 2 | 0.0 | 0.0 | 2 | 0.1 | 0.1 | 2 | 0.0 | 0.0 | 2 | 0.0 | 0.0 | 2 | 0.1 | 0.1 | 2 |
| ID NO: 866) | | | | | | | | | | | | | | | | | | | |
| ST038878 (SEQ | DNA::RNA T3-T2 | 0.5 | 0.1 | 2 | 0.1 | 0.1 | 2 | 0.2 | 0.1 | 2 | 0.1 | 0.1 | 2 | 0.0 | 0.0 | 2 | 0.2 | 0.2 | 2 |
| ID NO: 867) | | | | | | | | | | | | | | | | | | | |
| ST038879 (SEQ | DNA::RNA T2-T1 | 1.5 | 0.2 | 2 | 0.1 | 0.1 | 2 | 0.1 | 0.1 | 2 | 0.3 | 0.2 | 2 | 0.0 | 0.0 | 2 | 0.4 | 0.2 | 2 |
| ID NO: 868) | | | | | | | | | | | | | | | | | | | |
| ST038880 (SEQ | DNA::RNA T1-D1 | 5.3 | 0.1 | 2 | 0.3 | 0.1 | 2 | 0.2 | 0.1 | 2 | 2.4 | 0.4 | 2 | 0.3 | 0.2 | 2 | 0.6 | 0.1 | 2 |
| ID NO: 869) | | | | | | | | | | | | | | | | | | | |
| ST038814 (SEQ | DNA::RNA D1-D2 | 8.9 | 0.8 | 2 | 0.3 | 0.0 | 2 | 0.2 | 0.0 | 2 | 5.6 | 0.6 | 2 | 0.2 | 0.1 | 2 | 0.1 | 0.1 | 2 |
| ID NO: 487) | | | | | | | | | | | | | | | | | | | |
| ST038881 (SEQ | DNA::RNA D2-D3 | 11.9 | 1.9 | 2 | 0.9 | 0.5 | 2 | 0.6 | 0.0 | 2 | 6.7 | 1.6 | 2 | 0.3 | 0.0 | 2 | 0.7 | 0.2 | 2 |
| ID NO: 871) | | | | | | | | | | | | | | | | | | | |
| ST038882 (SEQ | DNA::RNA D3-D4 | 15.0 | 3.0 | 2 | 0.7 | 0.0 | 2 | 0.7 | 0.4 | 2 | 9.1 | 2.0 | 2 | 0.5 | 0.3 | 2 | 0.7 | 0.2 | 2 |
| ID NO: 872) | | | | | | | | | | | | | | | | | | | |
| ST038883 (SEQ | DNA::RNA D5-D6 | 9.3 | 1.2 | 2 | 0.4 | 0.2 | 2 | 0.3 | 0.2 | 2 | 7.4 | 0.9 | 2 | 0.2 | 0.0 | 2 | 0.5 | 0.0 | 2 |
| ID NO: 873) | | | | | | | | | | | | | | | | | | | |
| ST038884 (SEQ | DNA::RNA D7-D8 | 9.2 | 0.1 | 2 | 0.3 | 0.1 | 2 | 0.5 | 0.1 | 2 | 7.2 | 1.3 | 2 | 0.4 | 0.3 | 2 | 0.7 | 0.8 | 2 |
| ID NO: 874) | | | | | | | | | | | | | | | | | | | |
| ST038885 (SEQ | 20 nt Template | 12.3 | 1.6 | 2. | 0.6 | 0.1 | 2 | 0.3 | 0.0 | 2 | 7.4 | 1.8 | 2 | 0.4 | 0.1 | 2 | 0.6 | 0.2 | 2 |
| ID NO: 875) | | | | | | | | | | | | | | | | | | | |
| ST038886 (SEQ | 25 nt Template | 11.6 | 0.1 | 2 | 0.5 | 0.1 | 2 | 0.1 | 0.0 | 2 | 4.8 | 0.9 | 2 | 0.1 | 0.0 | 2 | 0.4 | 0.4 | 2 |
| ID NO: 876) | | | | | | | | | | | | | | | | | | | |
| ST038887 (SEQ | 30 nt Template | 14.4 | 5.6 | 2 | 0.9 | 0.1 | 2 | 0.4 | 0.3 | 2 | 5.5 | 1.0 | 2 | 0.4 | 0.0 | 2 | 0.9 | 0.1 | 2 |
| ID NO: 877) | | | | | | | | | | | | | | | | | | | |
| ST038888 (SEQ | 35 nt Template | 12.9 | 2.6 | 2 | 0.9 | 0.0 | 2 | 0.2 | 0.0 | 2 | 5.6 | 2.1 | 2 | 0.2 | 0.1 | 2 | 0.5 | 0.1 | 2 |
| ID NO: 878) | | | | | | | | | | | | | | | | | | | |
| ST038889 (SEQ | 19 nt DRS | 10.2 | 1.8 | 2 | 0.3 | 0.1 | 2 | 0.2 | 0.0 | 2 | 4.7 | 0.7 | 2 | 0.2 | 0.1 | 2 | 0.2 | 0.0 | 2 |
| ID NO: 879) | | | | | | | | | | | | | | | | | | | |
| ST038890 (SEQ | 15 nt DRS | 12.7 | 1.9 | 2 | 0.6 | 0.0 | 2 | 0.2 | 0.1 | 2 | 8.1 | 1.1 | 2 | 0.3 | 0.1 | 2 | 0.2 | 0.1 | 2 |
| ID NO: 880) | | | | | | | | | | | | | | | | | | | |
| ST038891 (SEQ | 13 nt DRS | 22.3 | 1.1 | 2 | 0.7 | 0.1 | 2 | 0.4 | 0.3 | 2 | 11.8 | 0.3 | 2 | 0.4 | 0.1 | 2 | 0.8 | 0.3 | 2 |
| ID NO: 881) | | | | | | | | | | | | | | | | | | | |
| ST038892 (SEQ | 12 nt DRS | 22.2 | 0.8 | 2 | 1.5 | 0.5 | 2 | 0.5 | 0.3 | 2 | 8.4 | 2.6 | 2 | 0.3 | 0.1 | 2 | 0.8 | 0.1 | 2 |
| ID NO: 882) | | | | | | | | | | | | | | | | | | | |
| ST038893 (SEQ | dU T3 | 0.3 | 0.2 | 2 | 0.0 | 0.0 | 2 | 0.5 | 0.5 | 2 | 0.0 | 0.0 | 2 | 0.0 | 0.0 | 2 | 0.4 | 0.6 | 2 |
| ID NO: 883) | | | | | | | | | | | | | | | | | | | |
| ST038894 (SEQ | dU D1 | 0.1 | 0.1 | 2 | 0.3 | 0.0 | 2 | 0.7 | 0.1 | 2 | 0.0 | 0.0 | 2 | 0.0 | 0.0 | 2 | 0.8 | 0.1 | 2 |
| ID NO: 884) | | | | | | | | | | | | | | | | | | | |
| ST038895 (SEQ | dU D18 | 9.8 | 2.1 | 2 | 0.4 | 0.2 | 2 | 0.1 | 0.1 | 2 | 4.5 | 0.6 | 2 | 0.2 | 0.1 | 2 | 0.4 | 0.1 | 2 |
| ID ON: 885) | | | | | | | | | | | | | | | | | | | |
| ST038896 (SEQ | dU D1 & D19 | 0.0 | 0.0 | 2 | 0.2 | 0.1 | 2 | 0.3 | 0.1 | 2 | 0.0 | 0.0 | 2 | 0.1 | 0.1 | 2 | 0.7 | 0.1 | 2 |
| ID NO: 886) | | | | | | | | | | | | | | | | | | | |

### Example 32—Templated Editing at the VEGFA Locus in Huh7 Cells with 3′ Extensions Having Various DNA Incorporation Patterns

This Example assessed the editing efficiency of tgRNA incorporating different numbers of ribonucleotides (rNTP) in the template sequence and different numbers of deoxyribonucleotides in the DRS sequence. Indel analysis and editing analysis were performed using templated guide RNAs (tgRNAs) targeting the VEGFA locus.

Guide RNAs as described in Table 82 were delivered to Huh7 cells with either a SpyCas9 encoding mRNA (SEQ ID NO: 1001) to observe indels resulting from SpyCas9 cleavage activity, or an mRNA encoding an nCas9-polymerase fusion protein such as nCas9-T5Pol(D164A,E166A) (SEQ ID No. 1130) or nCas9-polK (SEQ ID No. 1022) as indicated in Table 82.

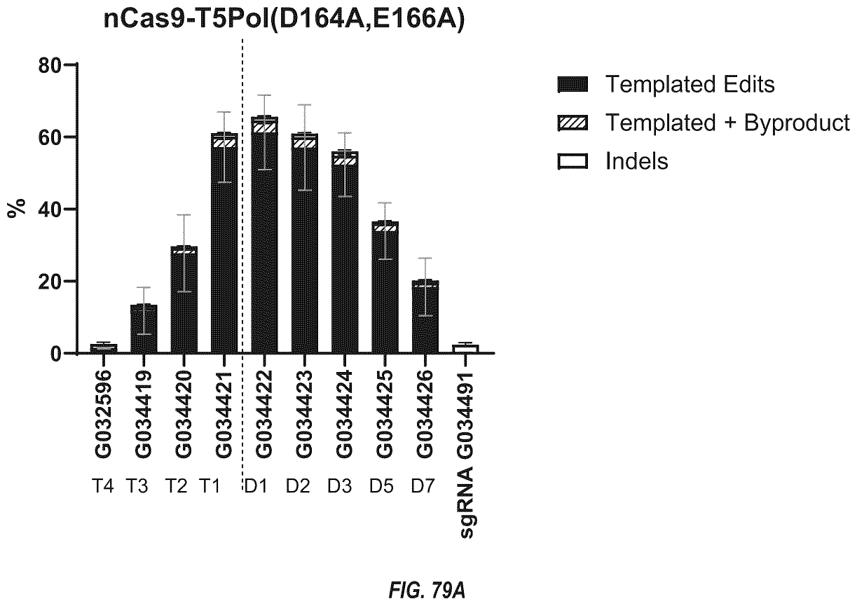

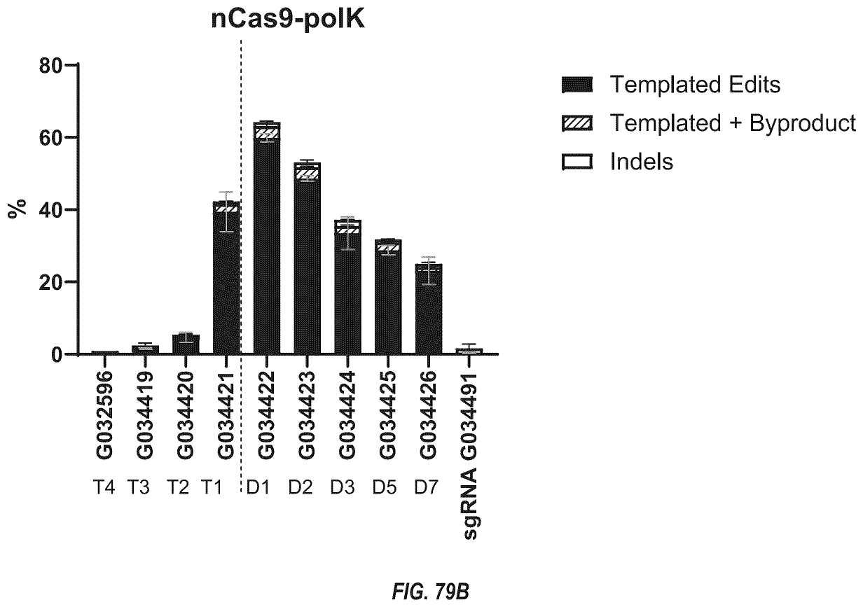

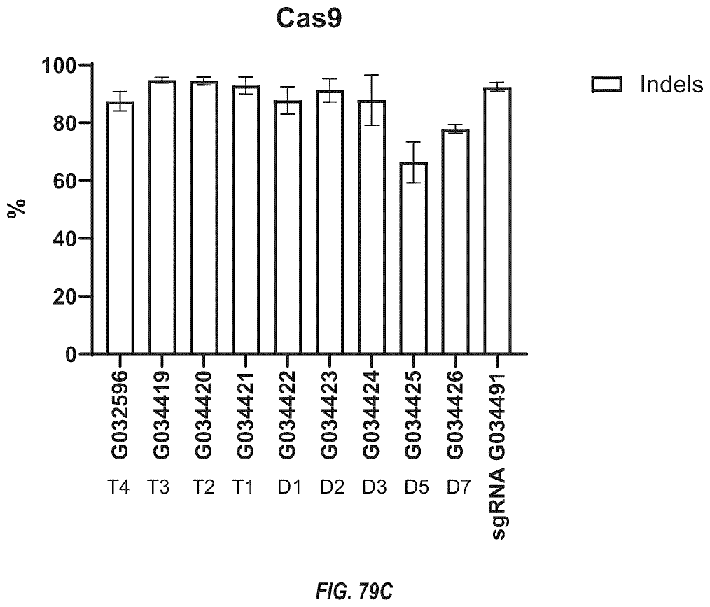

Huh7 cells were prepared as described in Example 1, and plated at a density of 15,000 cells/well. After 24 hours of incubation at 37° C., 5% CO₂, cells were transfected using Lipofectamine MessengerMAX Reagent (Invitrogen, Cat. LMRNA015) according to manufacturer's instructions with 150 ng of mRNA, 50 nM gRNA as indicated in Table X151. Samples were prepared in duplicate. After transfection and incubation at 37° C., 5% CO₂ for 72 hours, samples were prepared for next-generation sequencing (NGS) and analysis for editing efficiency as described in Example 1. Table 82 shows the editing data at the VEGFA locus for all mRNAs. FIG. 79A shows editing in cells treated with nCas9-T5Pol(D164A,E166A) (SEQ ID No. 1130). FIG. 79B shows editing in cells treated with nCas9-polK (SEQ ID No. 1022). FIG. 79C shows indel analysis in cells treated with SpyCas9 (SEQ ID No. 1001).

TABLE 82
Mean percent of sequencing reads with only templated edits, with templated edits and
byproduct edits, and with indels, as described in Example 1, for tgRNAs incorporating
deoxynuclotides at various positions relative to the template: DRS junction.

| | | nCas9-T5Pol(D164A, E166A) | | | | | | | | | | | | | |
|---|---|---|---|---|---|---|---|---|---|---|---|---|---|---|---|
| | | (SEQ ID NO: 1130) | | | | | | nCas9-polK (SEQ ID NO: 1022) | | | | | | Cas9 (SEQ | |
| | | Templated | | Templated + | | | | Templated | | Templated + | | | | ID NO: 1004) | |
| | Guide | Edit | | Byproduct | | Indels | | Edit | | Byproduct | | Indels | | Indels | |
| Guide | Descripción | Mean | SD | Mean | SD | Mean | SD | Mean | SD | Mean | SD | Mean | SD | Mean | SD |
| G032596 (SEQ | D1 dNTP | 0.6 | 0.0 | 0.0 | 0.0 | 0.3 | 0.0 | 1.4 | 0.1 | 0.2 | 0.1 | 1.1 | 0.4 | 87.5 | 3.3 |
| ID NO: 417) | | | | | | | | | | | | | | | |
| G034419 (SEQ | T1-T3 | 1.6 | 0.2 | 0.2 | 0.1 | 0.8 | 0.6 | 11.8 | 6.5 | 0.9 | 0.6 | 0.8 | 0.1 | 94.8 | 0.9 |
| ID NO: 418) | rNTP | | | | | | | | | | | | | | |
| G034420 (SEQ | T1-T2 | 4.7 | 1.4 | 0.3 | 0.0 | 0.4 | 0.1 | 27.8 | 10.7 | 1.5 | 0.4 | 0.5 | 0.1 | 94.5 | 1.4 |
| ID NO: 419) | rNTP | | | | | | | | | | | | | | |
| G034421 (SEQ | T1 rNTP | 39.4 | 5.5 | 2.2 | 0.1 | 0.8 | 0.1 | 57.2 | 9.8 | 3.0 | 0.8 | 1.0 | 0.2 | 92.9 | 3.0 |
| ID NO: 420) | | | | | | | | | | | | | | | |
| G034422 (SEQ | All | 59.9 | 1.1 | 3.3 | 0.6 | 1.1 | 0.3 | 61.3 | 10.3 | 3.4 | 0.9 | 1.0 | 0.3 | 87.8 | 4.7 |
| ID NO: 421) | template | | | | | | | | | | | | | | |
| | dNTP, all | | | | | | | | | | | | | | |
| | DRS rNTP | | | | | | | | | | | | | | |
| G034423 (SEQ | D1-D2 | 48.7 | 0.8 | 3.1 | 0.1 | 1.4 | 0.6 | 57.1 | 11.9 | 2.9 | 0.5 | 1.0 | 0.3 | 91.3 | 4.0 |
| ID NO: 422) | dNTP | | | | | | | | | | | | | | |
| G034424 (SEQ | D1-D3 | 33.5 | 4.5 | 2.0 | 0.3 | 1.8 | 0.1 | 52.4 | 8.8 | 2.5 | 0.2 | 1.2 | 0.4 | 87.9 | 8.7 |
| ID NO: 423) | dNTP | | | | | | | | | | | | | | |
| G034425 (SEQ | D1-D5 | 28.8 | 1.3 | 2.2 | 0.1 | 0.8 | 0.1 | 34.0 | 7.8 | 2.2 | 0.6 | 0.6 | 0.1 | 66.3 | 7.1 |
| ID NO: 424) | dNTP | | | | | | | | | | | | | | |
| G034426 (SEQ | D1-D7 | 23.1 | 3.8 | 1.0 | 0.1 | 0.9 | 0.4 | 18.5 | 8.0 | 1.2 | 0.6 | 0.6 | 0.2 | 77.9 | 1.5 |
| ID NO: 425) | dNTP | | | | | | | | | | | | | | |
| G034622 (SEQ | ngRNA | 0.2 | 0.2 | 0.0 | 0.0 | 0.0 | 0.0 | 0.1 | 0.1 | 0.0 | 0.0 | 0.0 | 0.0 | 0.6 | 0.2 |
| ID NO: 527) | | | | | | | | | | | | | | | |
| G034491 (SEQ | sgRNA | 0.4 | 0.1 | 0.0 | 0.0 | 1.3 | 1.2 | 0.0 | 0.0 | 0.1 | 0.1 | 2.4 | 0.5 | 92.4 | 1.6 |
| ID NO: 482) | | | | | | | | | | | | | | | |
| Untreated | N/A | 0.0 | 0.0 | 0.0 | 0.0 | 0.0 | 0.0 | | | | | | | | |

### Example 33—Templated Editing in Primary Cynomolgus Hepatocyte (PCH) Cells in the Presence of Enhancers of Template-Based Editing and T5 DNA Polymerase

This Example evaluated templated editing efficiency in the presence of various accessory factors (i.e., editing enhancers) in primary cynomolgus hepatocytes (PCH). The editing accessory factors assessed were HIV-Vpx-1, SIV-Vpx, M97, huFEN1, cynoFEN1, and MLH1-NTD.

Editing accessory factors, HIV-Vpx-1 (SEQ ID NO: 1501), SIV-Vpx (SEQ ID NO: 1585), M97 (SEQ ID NO: 1497), huFEN1 (SEQ ID NO: 1506), cynoFEN1 (SEQ ID NO: 1477), and MLH1-NTD (SEQ ID NO: 1549), were delivered into primary cynomolgus hepatocytes (PCH) in conjunction with a tgRNA (G037455; SEQ ID NO: 426) comprising a spacer sequence targeting the TTR locus and a 3′ extension encoding a TG-to-AC edit, resulting in a STOP codon, and a mRNA encoding nCas9-T5pol(v3) (SEQ ID NO: 1222).

PCH (Gibco, Lot CY423) were prepared as described in Example 1, and plated on a collagen I coated 96-well plate (Stem cell technologies, Cat. 100-0366) at a density of 40,000 cells/well. LNPs comprising a single RNA species were used for lipofection. Each LNP contained one of an mRNA encoding an editing enhancer (HIV-Vpx-1, SIV-Vpx, M97, huFEN1, cynoFEN1, or MLH1); an mRNA encoding nCas9-T5pol(v3); or a tgRNA (G037455). The LNPs were prepared as described in Example 1. The LNPs were made with a molar ratio of 50% Lipid A, 38% cholesterol, 9% DSPC, and 3% PEG2k-DMG, and formulated with a lipid amine to RNA phosphate (N:P) molar ratio of about 6. Cells were incubated at 37° C., 5% CO₂ for 24 hours prior to treatment with LNPs. LNPs were incubated in media containing 10% fetal bovine serum (FBS) at 37° C. for 10 minutes. Post-incubation, the LNPs were added to the PCH as follows. The guide RNA and the mRNA encoding nCas9-T5pol(v3) were delivered to the cells in a tgRNA:mRNA ratio of 1:2 by weight, in an 8-point 3-fold dilution series starting at 200 ng total LNP. For each dilution of the guide RNA and mRNA encoding the nCas9-T5pol(v3) fusion protein, an mRNA encoding an editing enhancer (HIV-Vpx-1, SIV-Vpx, M97, huFEN1, cynoFEN1, or MLH1) was added at a fixed concentration of 100 ng. Control groups were also included in which no editing enhancer was added. The cells were lysed 72 hours post-treatment for NGS analysis as described in Example 1. Samples were run in triplicates, unless otherwise indicated.

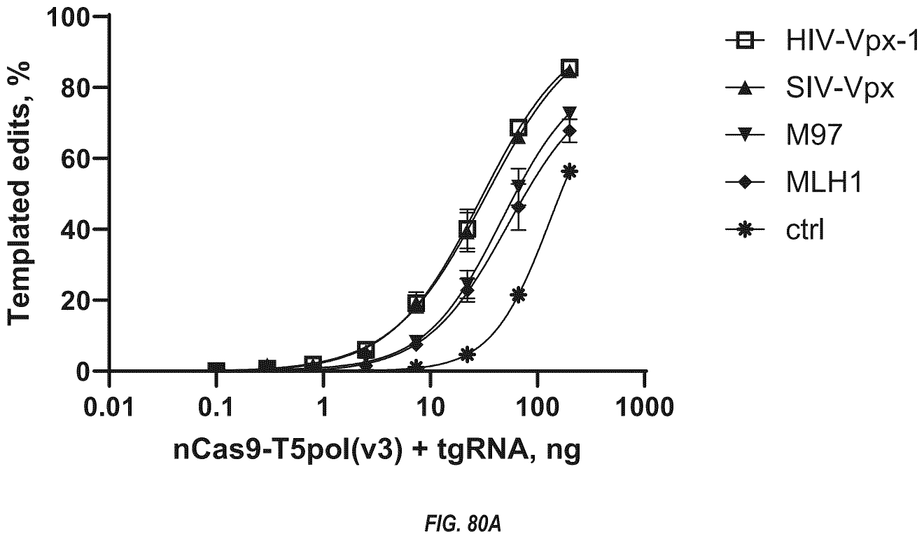

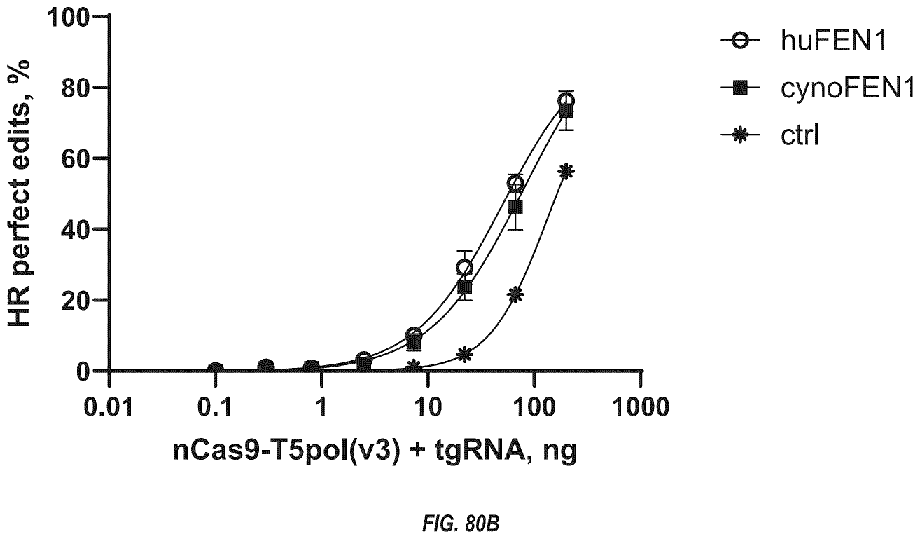

FIGS. 80A-80B and Table 83 show editing efficiencies obtained at the TTR locus in PCH treated with nCas9-T5pol(v3) and G037455, in the presence of HIV-Vpx-1, SIV-Vpx, M97, huFEN1, cynoFEN1, or MLH1.

TABLE 83
Templated editing at the TTR locus in PCH treated with nCas9-T5pol(v3) and G037455,
in the presence of HIV-Vpx-1, SIV-Vpx, M97, huFEN1, cynoFEN1, or MLH1

| | | nCas9-T5pol(v3) and G037455 (2:1 ratio) (ng) | | | | | | | |
|---|---|---|---|---|---|---|---|---|---|
| | | 200 | 66.7 | 22.2 | 7.4 | 2.5 | 0.8 | 0.3 | 0.1 |
| Control | Editing % | 56.35* | 21.53 | 4.70* | 1.05* | 0.15* | 0.00* | 0.00* | 0.00* |
| (no editing | SD | 0.49* | 1.74 | 1.27* | 0.07* | 0.07* | 0.00* | 0.00* | 0.00* |
| enhancer) | | | | | | | | | |
| HIV-Vpx-1- | Editing % | 85.60 | 68.67 | 40.10* | 19.23 | 6.00 | 1.83 | 0.67 | 0.00 |
| | SD | 2.25 | 1.46 | 5.52* | 1.86 | 0.72 | 0.47 | 0.12 | 0.00 |
| SIV-Vpx | Editing % | 84.67 | 66.13 | 39.20 | 19.37 | 5.50 | 1.70 | 1.80 | 0.00 |
| | SD | 1.51 | 1.00 | 5.48 | 2.90 | 1.15 | 0.40 | 0.44 | 0.00 |
| M97 | Editing % | 72.63 | 51.97 | 24.45* | 8.17 | 3.87 | 0.33 | 0.37 | 0.00 |
| | SD | 1.93 | 5.21 | 3.89* | 0.81 | 0.67 | 0.06 | 0.29 | 0.00 |
| huFEN1 | Editing % | 76.20 | 52.97 | 29.20 | 10.07 | 3.10 | 0.80 | 1.07 | 0.00 |
| | SD | 2.93 | 2.48 | 4.65 | 0.74 | 1.65 | 0.44 | 0.15 | 0.00 |
| cynoFEN1 | Editing % | 73.47 | 46.20 | 23.73 | 8.10 | 1.90 | 0.77 | 0.80 | 0.00 |
| | SD | 5.54 | 6.44 | 3.79 | 2.36 | 0.80 | 0.23 | 0.17 | 0.00 |
| MLH1 | Editing % | 67.80* | 46.27 | 22.77 | 7.43 | 1.50 | 0.37 | 0.00 | 0.00* |
| | SD | 3.25* | 6.55 | 3.25 | 1.46 | 0.17 | 0.21 | 0.00 | 0.00* |

*N = 2

### Example 34—Templated Editing in Primary Cynomolgus Hepatocytes in the Presence of FEN1 and T5 DNA Polymerase

This Example evaluated templated editing efficiency in the presence of various accessory factors (i.e., editing enhancers) in primary cynomolgus hepatocytes (PCH). The editing accessory factor FEN1 or FEN1 fused to a KI domain were assessed in this Example.

An mRNA encoding one of FEN1 (SEQ ID NO: 1506), or a FEN1 fused to a KI (FEN1-KI, SEQ ID NO: 1487), was delivered into primary cynomolgus hepatocytes (PCH) in conjunction with a tgRNA (G037455; SEQ ID NO: 426) comprising a spacer sequence targeting the TTR locus and a 3′ extension encoding a TG-to-AC edit, resulting in a STOP codon, and a mRNA encoding one of nCas9-T5pol(v3) (SEQ ID NO: 1222), or a nCas9-T5Pol fusion protein comprising an EI sequence (nCas9-T5-EI, SEQ ID NO: 1251).

PCH (Gibco, Lot CY423) were prepared as described in Example 1, and plated on a collagen I coated 96-well plate (Stem cell technologies, Cat. 100-0366) at a density of 40,000 cells/well. LNPs comprising a single RNA species were used for lipofection. Each LNP contained an mRNA encoding one of FEN1 or FEN1-KI; an mRNA encoding one of nCas9-T5pol(v3) or nCas9-T5-EI; or a tgRNA (G037455). The LNPs were prepared as described in Example 1. The LNPs were made with a molar ratio of 50% Lipid A, 38% cholesterol, 9% DSPC, and 3% PEG2k-DMG, and formulated with a lipid amine to RNA phosphate (N:P) molar ratio of about 6. Cells were incubated at 37° C., 5% CO₂ for 24 hours prior to treatment with LNPs. LNPs were incubated in media containing 10% fetal bovine serum (FBS) at 37° C. for 10 minutes. Post-incubation, the LNPs were added to the PCH as follows. The guide RNA and the mRNA encoding nCas9-T5pol(v3) or nCas9-T5-EI were delivered to the cells in a tgRNA:mRNA ratio of 1:2 by weight, in an 8-point 3-fold dilution series starting at 200 ng total LNP. For each dilution of the guide RNA and mRNA encoding the nCas9-T5pol(v3) or nCas9-T5-EI, an mRNA encoding either one of FEN1 or FEN1-KI was added at a fixed concentration of 100 ng. Control groups were also included in which no editing enhancer was added. The cells were lysed 72 hours post-treatment for NGS analysis as described in Example 1. Samples were run in triplicates, unless otherwise indicated.

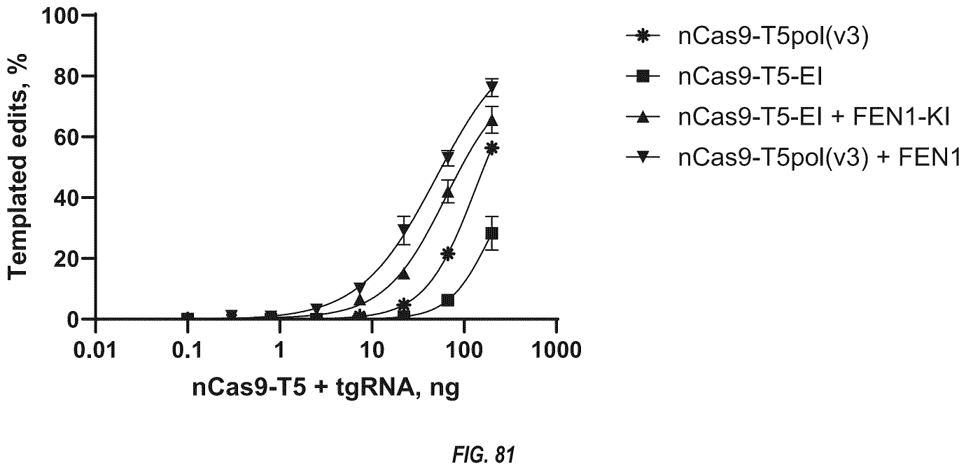

FIG. 81 and Table 84 show editing efficiencies obtained at the TTR locus in PCH treated with G037455 and with nCas9-T5pol(v3) or nCas9-T5-EI, in the presence of FEN1 or FEN1-KI.

TABLE 84
Templated editing at the TTR locus in PCH treated
with G037455 and with nCas9-T5pol(v3) or nCas9-
T5-EI, in the presence of FEN1 or FEN1-KI.

| | Total (ng) tgRNA + | | |
|---|---|---|---|
| mRNA species | nCas9-T5 mRNA | Mean % Editing | SD |
| nCas9-T5pol(v3) | 200 | 56.35* | 0.49* |
| | 66.7 | 21.53 | 1.74 |
| | 22.2 | 4.70* | 1.27* |
| | 7.4 | 1.05* | 0.07* |
| | 2.5 | 0.15* | 0.07* |
| | 0.8 | 0.00* | 0.00* |
| | 0.3 | 0.00* | 0.00* |
| | 0.1 | 0.00* | 0.00* |
| nCas9-T5-EI | 200 | 28.30* | 5.52* |
| | 66.7 | 6.30 | 1.57 |
| | 22.2 | 0.85* | 0.78* |
| | 7.4 | 0.15* | 0.07* |
| | 2.5 | 0.00* | 0.00* |
| | 0.8 | 0.00* | 0.00* |
| | 0.3 | no data | no data |
| | 0.1 | 0.03 | 0.06 |
| nCas9-T5-EI + | 200 | 65.65* | 4.45* |
| FEN1-KI | 66.7 | 42.05* | 3.75* |
| | 22.2 | 15.15* | 1.91* |
| | 7.4 | 6.65* | 0.78* |
| | 2.5 | 1.05* | 1.20* |
| | 0.8 | 0.50** | N/A |
| | 0.3 | 0.17 | 0.21 |
| | 0.1 | 0.00* | 0.00* |
| nCas9-T5pol(v3) + | 200 | 76.20 | 2.93 |
| FEN1 | 66.7 | 52.97 | 2.48 |
| | 22.2 | 29.20 | 4.65 |
| | 7.4 | 10.07 | 0.74 |
| | 2.5 | 3.10 | 1.65 |
| | 0.8 | 0.80 | 0.44 |
| | 0.3 | 1.07 | 0.15 |
| | 0.1 | 0.00 | 0.00 |

*N = 2;
**N = 1

### Example 35—Templated Editing in Primary Cynomolgus Hepatocytes in the Presence of Enhancers of Template-Based Editing with T5 DNA Polymerase

This Example evaluated templated editing efficiency in the presence of a combination of accessory factors (i.e., editing enhancers) in primary cynomolgus hepatocytes (PCH). The editing accessory factors assessed were a combination of FEN1 and Vpx.

Primary cynomolgus hepatocytes (PCH) were treated with a nCas9-T5pol(v2) (SEQ ID NO: 1217) fusion protein and a tgRNA (G037455; SEQ ID NO: 426) comprising a spacer sequence targeting the TTR locus and a 3′ extension encoding a TG-to-AC edit, resulting in a STOP codon, in the presence of editing accessory factors Vpx (SEQ ID NO: 1501) and FEN1 (SEQ ID NO: 1506).

PCH (Gibco, Lot CY423) were prepared as described in Example 1, and plated on a collagen I coated 96-well plate (Corning, Cat. 354407) at a density of 50,000 cells/well. LNPs comprising a single RNA species were used for lipofection. Each LNP contained one of an mRNA encoding nCas9-T5pol(v2), an mRNA encoding an editing enhancer (Vpx or FEN1), or a tgRNA (G037455). The LNPs were prepared as described in Example 1. The LNPs were made with a molar ratio of 50% Lipid A, 38% cholesterol, 9% DSPC, and 3% PEG2k-DMG, and formulated with a lipid amine to RNA phosphate (N:P) molar ratio of about 6. Cells were incubated at 37° C., 5% CO₂ for 24 hours prior to treatment with LNPs. LNPs were incubated in media containing 10% fetal bovine serum (FBS) at 37° C. for 10 minutes. Post-incubation, the LNPs were added to the PCH as follows. The guide RNA was delivered to the cells in an 8-point 3-fold dilution series starting at 300 ng total LNP. For each dilution of the guide RNA, 200 ng of an mRNA encoding the nCas9-T5pol(v2) fusion protein, 25 ng of an mRNA encoding Vpx, and 25 ng of an mRNA encoding FEN1 were also delivered to the cells. The cells were lysed 72 hours post-treatment for NGS analysis as described in Example 1. Samples were run in duplicates. An additional computational analysis was conducted on the NGS data to quantify reads containing a stop codon (i.e., percent reads with stop codon relative to total reads).

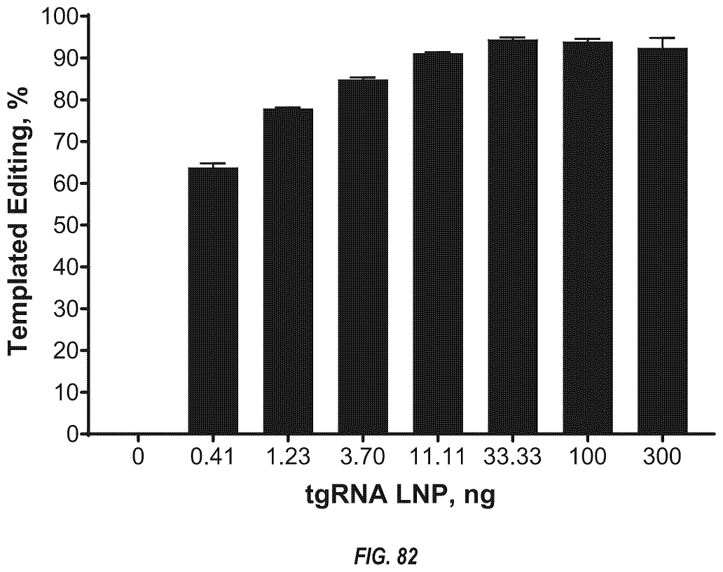

FIGS. 82 and Table 85 show editing efficiencies obtained at the TTR locus in PCH treated with nCas9-T5pol(v2), Vpx, and FEN1, at different dilutions of G037455.

TABLE 85
Editing at the TTR locus in PCH treated with nCas9-T5pol(v2), in
the presence of FEN1 and Vpx, with different amounts of G037455.

| | | | Templated + | | | | | |
|---|---|---|---|---|---|---|---|---|
| | Templated | | Byproduct | | | | STOP | |
| tgRNA | Edits % | | Edits % | | Indels % | | codon % | |
| (ng) | Mean | SD | Mean | SD | Mean | SD | Mean | SD |
| 300 | 92.45 | 2.33 | 1.40 | 0.14 | 0.35 | 0.07 | 93.65 | 2.33 |
| 100 | 93.95 | 0.64 | 1.35 | 0.35 | 0.40 | 0.42 | 94.95 | 0.64 |
| 33.33 | 94.45 | 0.49 | 1.25 | 0.07 | 0.35 | 0.07 | 95.55 | 0.64 |
| 11.11 | 91.15 | 0.21 | 1.05 | 0.21 | 0.30 | 0.14 | 92.00 | 0.28 |
| 3.7 | 84.85 | 0.49 | 1.25 | 0.07 | 0.45 | 0.07 | 85.80 | 0.28 |
| 1.23 | 77.95 | 0.21 | 1.15 | 0.07 | 0.30 | 0.00 | 78.90 | 0.42 |
| 0.41 | 63.80 | 0.99 | 0.75 | 0.07 | 0.20 | 0.00 | 64.55 | 0.92 |
| 0 | 0.00 | 0.00 | 0.00 | 0.00 | 0.00 | 0.00 | 0.25 | 0.07 |

### Example 36—In Vivo Editing at the DNMT1 Locus in Mouse Liver with Polymerase Kappa Delivered Using LNPs with Different Formulations

This Example evaluated in vivo templated editing efficiency in mice. Further, the editing efficiency at the DNMT1 locus in mouse liver was evaluated using polymerase Kappa delivered via LNPs prepared using different lipid components with different molar compositions.

LNPs comprising an mRNA encoding nCas9-PolK (1-526, GE411-412RV) (SEQ ID NO: 1232) and a nicking guide RNA (ngRNA, G035727; SEQ ID NO: 428), along with a template guide RNA (tgRNA, G034987; SEQ ID NO: 427) comprising a spacer sequence targeting the mouse DNMT1 locus and a 3′ extension encoding a G-to-C edit, were prepared as described in Example 1, with a ngRNA: tgRNA:mRNA ratio of 2:3:10 by mass, and the following molar compositions:
LNP #1 was prepared with a molar ratio of 50% Lipid A, 38% cholesterol, 9% DSPC, and 3% PEG2k-DMG,
LNP #2 was prepared with a molar ratio of 50% Lipid B, 38% cholesterol, 9% DSPC, and 3% methoxy-PEG2000-carbamoyl-1,2-tridecyoxypropylamine (also referred to herein as “C13 Ether PEG lipid”),
LNP #3 was prepared with a molar ratio of 50% Lipid C, 38% cholesterol, 9% DSPC, and 3% C13 Ether PEG lipid,
I LNP #4 was prepared with a molar ratio of 50% Lipid D, 38.5% cholesterol, 10% DSPC, and 1.5% C13 Ether PEG lipid, and
LNP #5 was prepared with a molar ratio of 50% Lipid E, 38.5% cholesterol, 10% DSPC, and 1.5% C13 Ether PEG lipid.

The chemical names corresponding to lipids used in the LNP formulations above are listed in Table 86.

TABLE 86
Exemplary ionizable lipids used in LNP formulations described herein.

| LNP | | | |
|---|---|---|---|
| formu- | Ionizable | | |
| lation | lipid | Chemical name | Chemical Formula |
| LNP#1 | Lipid A | (9Z,12Z)-3- ((4,4-bis(oct- yloxy)butano- yl)oxy)-2-((((3- (diethylamino)- propoxy)carbon- yl)oxy)methyl)- propyl octadeca- 9,12-dienoate | 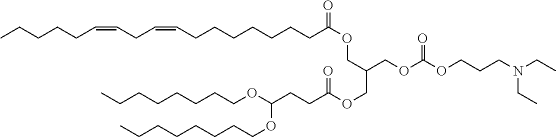 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| LNP#2 | Lipid B | O,O′-(2-((((2- (diethylamino)- ethyl)carbamo- yl)oxy)methyl)- propane-1,3- diyl) di(hepta- decan-9-yl)- diglutarate | 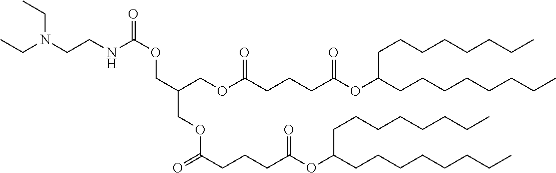 [SMILES: CCCCCCCCC(CCCCCCCC)OC(=O)CCCC(=O)OCC(COC(=O)CCCC(=O)OC(CCCCCCCC)CCCCCCCC)COC(=O)NCCN(CC)CC] |
| LNP#3 | Lipid C | 3-(((2-(azepan- 1-yl)ethyl)- carbamoyl)- oxy)-2- ((((9Z,12Z)- octadeca-9,12- dienoyl)oxy)- methyl)propyl heptadecan-9- yl glutarate | 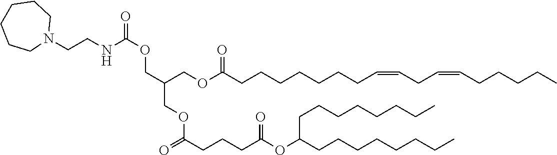 [SMILES: CCCCC/C=C\C/C=C\CCCCCCCC(=O)OCC(COC(=O)CCCC(=O)OC(CCCCCCCC)CCCCCCCC)COC(=O)NCCN1CCCCCC1] |
| LNP#4 | Lipid D | 2-((4-(((3-(pyr- rolidin-1-yl)- propoxy)carbon- yl)oxy)decano- yl)oxy)propane- 1,3-diyl (9Z,9′Z,12Z, 12′Z)-bis(octa- deca-9,12- dienoate) | 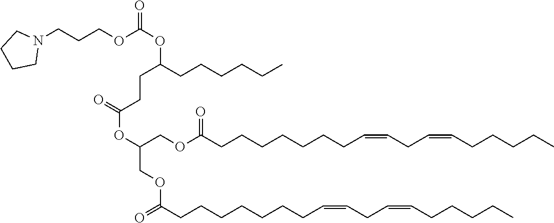 [SMILES: CCCCC/C=C\C/C=C\CCCCCCCC(=O)OCC(COC(=O)CCCCCCC/C=C\C/C=C\CCCCC)OC(=O)CCC(CCCCCC)OC(=O)OCCCN1CCCC1] |
| LNP#5 | Lipid E | 8-((8,8-bis- (oct-yloxy)oct- yl)(2-hydroxy- | 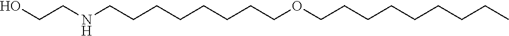 [SMILES: CCCCCCCCCOCCCCCCCCNCCO] |
| | | ethyl)amino- | |
| | | octyl nonanoate | |

Female C57Bl/6J mice, six to seven weeks of age, were dosed with LNPs based on individual body weight measured the morning of dosing. LNPs were dosed via the lateral tail vein at a dose of 3 mpk. The number of animals in each group was N=5. The animals were periodically observed for adverse effects for at least 24 hours post dose.

Animals were euthanized seven days post-dose by exsanguination via cardiac puncture under isoflurane anesthesia. Liver tissue was collected, and genomic DNA was isolated from the liver tissue for editing measurement by NGS, as described in Example 1.

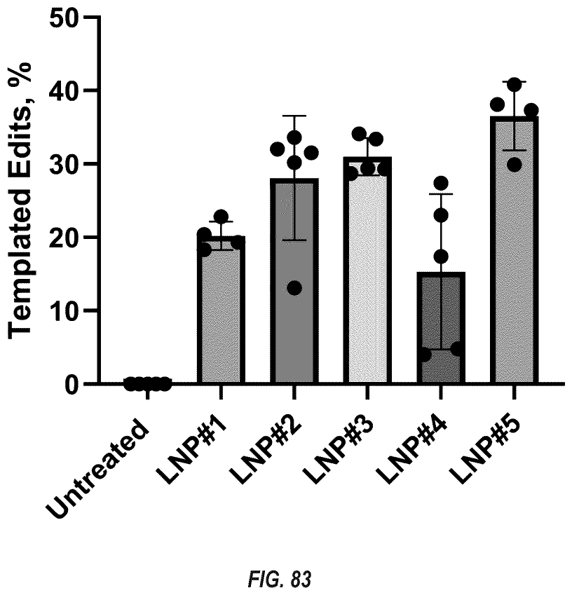

Table 87 and FIG. 83 show editing efficiencies at the DNMT1 locus in mice treated with the LNPs as described above.

TABLE 87
Editing efficiencies at the DNMT1 locus in mice treated
with LNPs comprising an mRNA encoding nCas9-PolK(1-526,
GE411-412RV), a ngRNA (G035727) and a tgRNA (G034987).

| | | | Templated + | | | |
|---|---|---|---|---|---|---|
| | Templated | | Byproduct | | | |
| | Edits % | | Edits % | | Indels % | |
| | Mean | SD | Mean | SD | Mean | SD |
| Untreated | 0.00 | 0.00 | 0.00 | 0.00 | 0.00 | 0.00 |
| LNP#1 | 20.20* | 1.93* | 0.63* | 0.13* | 0.45* | 0.06* |
| LNP#2 | 28.08 | 8.46 | 1.00 | 0.33 | 0.96 | 0.32 |
| LNP#3 | 30.98 | 2.55 | 1.02 | 0.08 | 0.78 | 0.08 |
| LNP#4 | 15.32 | 10.58 | 0.40 | 0.31 | 0.28 | 0.23 |
| LNP#5 | 36.53* | 4.66* | 1.00* | 0.22* | 0.73* | 0.24* |

*N = 4

### Example 37—Expression of nCas9-Polymerase Fusion Protein in Rat Liver Following Delivery of mRNAs Encoding Polymerase and SpyCas9 Nickase in Trans

This Example evaluated whether an nCas9-polymerase fusion protein can be expressed in vivo following the delivery in trans of an mRNA encoding a SpyCas9 nickase operably linked to a split intein, and a mRNA encoding a polymerase operably linked to a complementary split intein. The exemplary split inteins evaluated in this Example were CfaN (e.g., linked to nCas9) and CfaC (e.g., linked to a DNA polymerase).

LNPs were prepared as described in Example 1 and formulated with a lipid amine to RNA phosphate (N:P) molar ratio of about 6, with the following formulations:
LNP #6 was prepared with a molar ratio of 50% Lipid A, 38% cholesterol, 9% DSPC, and 3% PEG2k-DMG lipid,
LNP #7 and LNP #9 were each prepared with a molar ratio of 50% Lipid B, 38% cholesterol, 9% DSPC, and 3% C13 Ether PEG lipid, and
LNP #8 was prepared with a molar ratio of 50% Lipid C, 38% cholesterol, 9% DSPC, and 3% C13 Ether PEG lipid.

LNPs #6, 7, and 8 each comprised (i) an mRNA encoding a nCas9-CfaN (SEQ ID NO: 1362) fusion protein comprising a nCas9 moiety and a CfaN split intein, and (ii) an mRNA encoding a CfaC-PolK(1-526, GE411-412RV) fusion protein (SEQ ID NO: 1368) comprising a CfaC split intein capable of undergoing a trans-splicing reaction with a CfaN split intein, and a PolK variant moiety, in a nCas9-CfaN: CfaC-PolK(1-526, GE411-412RV) mass ratio of 5:1. LNP #9 comprised an mRNA encoding nCas9-PolKv2 (SEQ ID NO: 1371).

Female Sprague Dawley rats (Charles River Labs), six weeks of age, were dosed with a LNP as described above, based on individual body weight measured the morning of dosing. LNPs were dosed via the lateral tail vein at a dose of 1 mpk of total RNA. The number of animals in each group was N=3. Animals were euthanized 2.5 hours post-dose by exsanguination via cardiac puncture under isoflurane anesthesia. Liver tissue was collected, and protein was extracted by using RIPA buffer (+Halt inhibitor 100×) to homogenize liver tissue at 100 mg tissue per ml of lysis buffer.

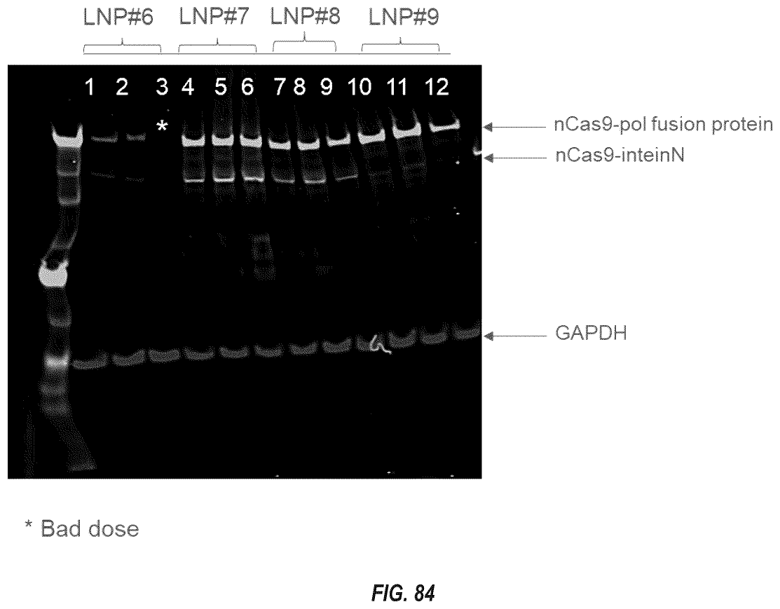

A Western blot was performed with the protein lysates and anti-SpyCas9 antibodies (Genscript, catalog #U0912VYTG0-4/P14IK001). A GAPDH positive control was used in each well. The gel was visualized under infrared light at 700 and 800 nm. The results of the gel are shown in FIG. 84.

### Example 38—Templated Editing at the TTR Locus in Primary Cynomolgus Hepatocytes Using a Polymerase Delivered in Trans with a SpyCas9 Nickase

This Example compared the editing efficiency of nCas9-polymerase constructs delivered in a split configuration and recombined by inteins, to that of a full-length nCas9-polymerase construct delivered as a single fusion protein. A SpyCas9 H840A nickase (nCas9) and a polymerase (e.g., polymerase Kappa variant, or T5 polymerase variant) were delivered into primary cynomolgus hepatocytes (PCH) in cis (as a fusion protein) or in trans (using inteins), along with a mRNA encoding Vpx, and a tgRNA (G036237; SEQ ID NO: 477) comprising (i) a spacer sequence targeting the TTR locus, (ii) a scaffold with the structure shown in FIG. 6D, and (iii) a 3′ extension comprising a template encoding a TG-to-AC edit, resulting in a STOP codon, and a DRS.

PCH (Gibco, Lot CY423) were prepared as described in Example 1, and plated on a collagen I coated 96-well plate (Stem cell technologies, Cat. 100-0366) at a density of 40,000 cells/well. PCH were lipofected using pre-mixed lipid formulations with a molar composition of 50% Lipid A, 38% cholesterol, 9% DSPC, and 3% PEG2k-DMG as described in Example 1. Lipofections were performed with a tgRNA (G036237) at a fixed dose of 25 nM, and a mRNA load in a 5-point 2-fold dilution series starting at 400 ng total mRNA.

The mRNA transfected consisted of:
a mRNA encoding a nCas9-PolK (1-526, GE411-412RV) fusion protein (SEQ ID NO: 1232) comprising an nCas9 moiety and a polymerase Kappa variant moiety,
a mRNA encoding a nCas9-T5pol(v4) fusion protein (SEQ ID NO: 1374) comprising a nCas9 moiety and a T5 polymerase variant moiety, or
a combination, in a 1:1 mass ratio, of either:
   a mRNA encoding a nCas9-CfaN fusion protein (SEQ ID NO: 1377) comprising a nCas9 moiety and a CfaN split intein, and a mRNA encoding a CfaC-PolK(1-526, GE411-412RV) fusion protein (SEQ ID NO: 1383) comprising a CfaC split intein capable of undergoing a trans-splicing reaction with a CfaN split intein, and a polymerase Kappa variant moiety, or
   a mRNA encoding a nCas9-CfaN fusion protein (SEQ ID NO: 1377), and a mRNA encoding a CfaC-T5pol(v4) fusion protein (SEQ ID NO: 1380) comprising a CfaC split intein, and a T5 polymerase variant moiety.

In addition, the cells were also treated with an LNP, prepared as described in Example 1 with a molar ratio of 50% Lipid A, 38% cholesterol, 9% DSPC, and 3% PEG2k-DMG and comprising a mRNA encoding Vpx (SEQ ID NO: 1501), at a fixed dose of 50 ng/well. LNPs were incubated in media containing 10% fetal bovine serum (FBS) at 37° C. for 10 minutes before being added to the cells.

Cells were incubated at 37° C., 5% CO₂ for 24 hours prior to treatment with the pre-mixed lipid formulations and with the LNPs. The cells were lysed 72 hours post-treatment for NGS analysis as described in Example 1.

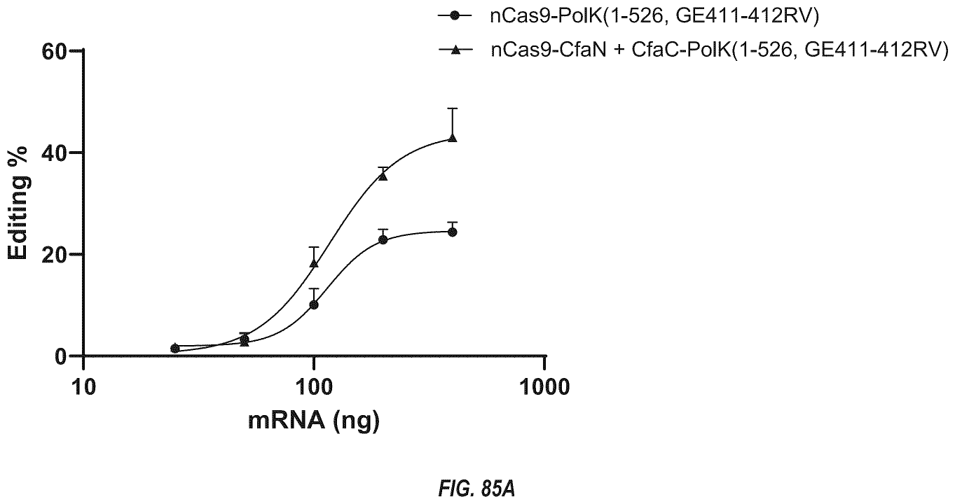

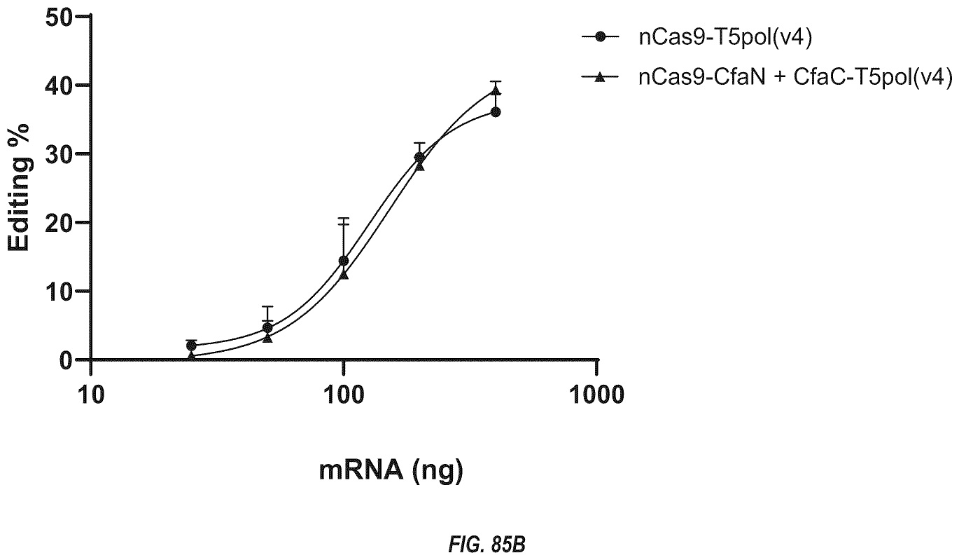

FIGS. 85A-B and Table 88 show editing efficiencies obtained at the TTR locus in PCH treated with a tgRNA (G036237), along with (i) nCas9-PolK (1-526, GE411-412RV), (ii) nCas9-T5pol(v4), (iii) a combination of nCas9-CfaN and Cfa-PolK (1-526, GE411-412RV), or (iv) a combination of nCas9-CfaN and Cfa-T5pol(v4), in the presence of Vpx.

TABLE 88
Editing at the TTR locus in PCH treated with a tgRNA (G036237),
along with nCas9-PolK(1-526, GE411-412RV), nCas9-T5pol(v4), or
a combination of either nCas9-CfaN and Cfa-PolK(1-526, GE411-412RV),
or nCas9-CfaN and Cfa-T5pol(v4), in the presence of Vpx.

| | | | total mRNA (ng) | | | | |
|---|---|---|---|---|---|---|---|
| | | | 400 | 200 | 100 | 50 | 25 |
| nCas9-PolK(1- | % Templated | mean | 24.35 | 22.88 | 10.1 | 3.3 | 1.48 |
| 526, GE411- | edits | SD | 1.98 | 2.05 | 3.19 | 1.28 | 0.56 |
| 412RV) | | N | 4 | 4 | 4 | 4 | 4 |
| | % Templated + | mean | 2.10 | 2.58 | 2.55 | N/A | N/A |
| | byproduct | SD | 0.57 | 0.42 | 0.31 | N/A | N/A |
| | edits | N | 4 | 4 | 4 | N/A | N/A |
| | % Indels | mean | 0.03 | 0.23 | 0.08 | N/A | N/A |
| | | SD | 0.05 | 0.26 | 0.15 | N/A | N/A |
| | | N | 4 | 4 | 4 | N/A | N/A |
| nCas9-CfaN + | % Templated | mean | 43.05 | 35.43 | 18.37 | 2.8 | 1.75 |
| CfaC-PolK(1- | edits | SD | 5.67 | 1.71 | 3.07 | 1.63 | 0.5 |
| 526, GE411- | | N | 4 | 4 | 3 | 4 | 4 |
| 412RV) | % Templated + | mean | 2.83 | 3.00 | 2.90 | N/A | N/A |
| | byproduct | SD | 0.21 | 0.55 | 1.30 | N/A | N/A |
| | edits | N | 4 | 4 | 4 | N/A | N/A |
| | % Indels | mean | 0.20 | 0.35 | 0.33 | N/A | N/A |
| | | SD | 0.22 | 0.26 | 0.58 | N/A | N/A |
| | | N | 4 | 4 | 3 | N/A | N/A |
| nCas9- | % Templated | mean | 36.10 | 29.50 | 14.43 | 4.70 | 2.05 |
| T5pol(v4) | edits | SD | 2.68 | 2.09 | 5.32 | 3.09 | 0.79 |
| | | N | 4 | 4 | 4 | 4 | 4 |
| | % Templated + | mean | 2.28 | 2.38 | 1.85 | N/A | N/A |
| | byproduct | SD | 0.36 | 0.64 | 0.38 | N/A | N/A |
| | edits | N | 4 | 4 | 4 | N/A | N/A |
| | % Indels | mean | 0.18 | 0.30 | 0.13 | N/A | N/A |
| | | SD | 0.17 | 0.48 | 0.25 | N/A | N/A |
| | | N | 4 | 4 | 4 | N/A | N/A |
| nCas9-CfaN + | % Templated | mean | 39.30 | 28.30 | 12.50 | 3.25 | 0.65 |
| CfaC- | edits | SD | 1.28 | 1.20 | 8.13 | 2.46 | 0.47 |
| T5pol(v4) | | N | 4 | 4 | 4 | 4 | 4 |
| | % Templated + | mean | 2.58 | 2.30 | 1.75 | N/A | N/A |
| | byproduct | SD | 0.54 | 0.74 | 0.31 | N/A | N/A |
| | edits | N | 4 | 4 | 4 | N/A | N/A |
| | % Indels | mean | 0.23 | 0.08 | 0.15 | N/A | N/A |
| | | SD | 0.45 | 0.15 | 0.30 | N/A | N/A |
| | | N | 4 | 4 | 4 | N/A | N/A |

### Example 39—Templated Editing at the DNMT1 Locus in Primary Mouse Hepatocytes Using a Polymerase Kappa Variant Delivered in Trans with a SpyCas9 Nickase in Different Ratios

This Example compared the editing efficiency of nCas9-polymerase constructs delivered in a split configuration and recombined by inteins, when different ratios of nCas9 to polymerase are used. A SpyCas9 H840A nickase (nCas9) and a polymerase Kappa variant were delivered into primary mouse hepatocytes (PMH) in cis (as a fusion protein) or in trans (using inteins), along with a tgRNA (G034987). The tgRNA comprised a spacer sequence targeting the DNMT1 locus, a scaffold with the structure shown in FIG. 6E, and a 3′ extension comprising a template encoding a G-to-C edit, and a DRS.

PMH (Gibco, Lot MC932) were prepared as described in Example 1, and plated on a collagen I coated 96-well plate (Stem cell technologies, Cat. 100-0366) at a density of 15,000 cells/well. LNPs comprising a single RNA species were used for lipofection. Each LNP contained one mRNA encoding one of:
a nCas9-PolK (1-526, GE411-412RV) fusion protein (SEQ ID NO: 1232) comprising a nCas9 moiety and a polymerase Kappa variant moiety,
a nCas9-CfaN fusion protein (SEQ ID NO: 1377) comprising a nCas9 moiety and a CfaN split intein,
a CfaC-PolK(1-526, GE411-412RV) fusion protein (SEQ ID NO: 1383) comprising a CfaC split intein capable of undergoing a trans-splicing reaction with a CfaN split intein, and a polymerase Kappa variant moiety, and
a tgRNA (G034987).

The LNPs were prepared as described in Example 1. The LNPs were made with a molar ratio of 50% Lipid A, 38% cholesterol, 9% DSPC, and 3% PEG2k-DMG, and formulated with a lipid amine to RNA phosphate (N:P) molar ratio of about 6. Cells were incubated at 37° C., 5% CO₂ for 24 hours prior to treatment with LNPs. LNPs were incubated in media containing 10% fetal bovine serum (FBS) at 37° C. for 10 minutes. Post-incubation, the LNPs were added to the PMH as follows. LNPs comprising tgRNA were added at a dose of 50 ng/well. In one condition, LNPs comprising mRNA encoding nCas9-PolK (1-526, GE411-412RV) fusion protein were delivered to the cells. In another condition, a combination of LNPs comprising an mRNA encoding nCas9-CfaN and LNPs comprising an mRNA encoding CfaC-PolK(1-526, GE411-412RV) were delivered to the cells in a nCas9-CfaN: CfaC-PolK(1-526, GE411-412RV) mass ratio of 5:1, 3:1, 1:1, or 1:3. LNPs in each condition were delivered to the cells in a 7-point 2-fold dilution series starting at 300 ng total mRNA. The cells were lysed 72 hours post-treatment for NGS analysis as described in Example 1. Samples were run in triplicates, unless otherwise indicated.

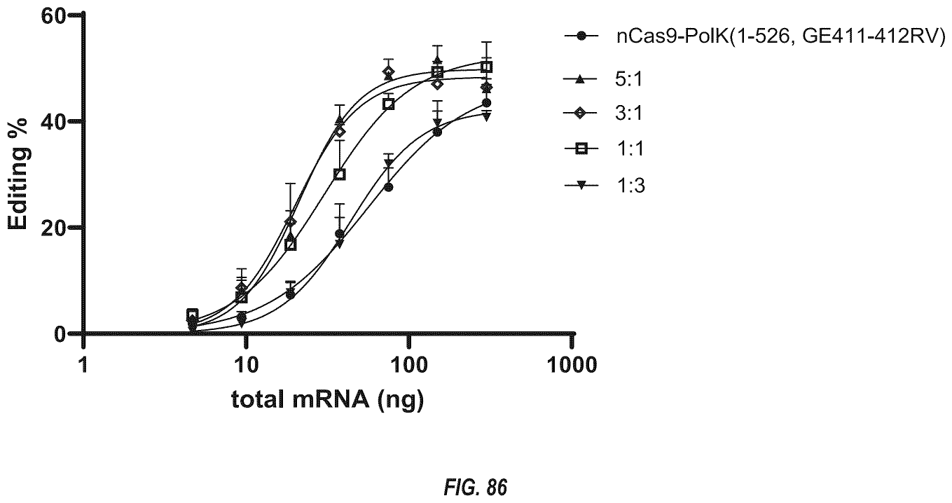

FIG. 86 and Table 89 show editing efficiencies obtained at the DNMT1 locus in PMH treated with a tgRNA (G034987), along with nCas9-PolK (1-526, GE411-412RV) or a combination of nCas9-CfaN and Cfa-PolK (1-526, GE411-412RV) in a nCas9-CfaN: CfaC-PolK(1-526, GE411-412RV) mass ratio of 5:1, 3:1, 1:1, or 1:3.

TABLE 89
Editing efficiencies at the DNMTI locus in PMH treated with a tgRNA (G034987), along with
nCas9-PolK(1-526, GE411-412RV) or a combination of nCas9-CfaN and Cfa-PolK(1-526, GE411-
412RV) in a nCas9-CfaN:CfaC-PolK(1-526, GE411-412RV) mass ratio of 5:1, 3:1, 1:1, or 1:3.

| | nCas9- | | | | | | | | | |
|---|---|---|---|---|---|---|---|---|---|---|
| | CfaN:CfaC- | | | | | | | | | |
| | PolK(1-26, | | | | | | | | | |
| | GE411-412RV) | | | total mRNA (ng) | | | | | | |
| | mass ratio | Edit Type | | 300 | 150 | 75 | 37.5 | 18.75 | 9.38 | 4.69 |
| nCas9- | N/A | % Templated | mean | 43.50 | 38.00 | 27.60 | 18.83 | 7.33 | 3.20 | 1.93 |
| PolK(1-526, | | edits | SD | 3.47 | 5.90 | 3.64 | 5.63 | 2.31 | 0.96 | 0.55 |
| GE411- | | % Templated + | mean | 0.70 | 0.57 | 0.57 | 0.27 | 0.07 | 0.03 | 0.00 |
| 412RV) | | Byproduct edits | SD | 0.00 | 0.06 | 0.21 | 0.21 | 0.06 | 0.06 | 0.00 |
| | | % Indels | mean | 0.00 | 0.00 | 0.00 | 0.00 | 0.00 | 0.20 | 0.00 |
| | | | SD | 0.00 | 0.00 | 0.00 | 0.00 | 0.00 | 0.35 | 0.00 |
| nCas9-CfaN + | 5:1 | % Templated | mean | 46.20 | 51.67 | 48.63 | 40.50 | 18.60* | 8.20 | 2.87 |
| CfaC-PolK(1- | | edits | SD | 1.82 | 2.59 | 0.06 | 2.57 | 2.12* | 2.44 | 0.06 |
| 526, GE411- | | % Templated + | mean | 0.67 | 0.87 | 0.90 | 0.80 | 0.23 | 0.10 | 0.07 |
| 412RV) | | Byproduct edits | SD | 0.06 | 0.12 | 0.10 | 0.10 | 0.15 | 0.10 | 0.06 |
| | | % Indels | mean | 0.00 | 0.03 | 0.00 | 0.00 | 0.07 | 0.00 | 0.00 |
| | | | SD | 0.00 | 0.06 | 0.00 | 0.00 | 0.12 | 0.00 | 0.00 |
| | 3:1 | % Templated | mean | 46.43 | 47.07 | 49.43 | 38.07 | 21.10* | 8.67 | 2.50 |
| | | edits | SD | 5.55 | 3.09 | 2.27 | 1.32 | 7.21* | 3.59 | 0.62 |
| | | % Templated + | mean | 0.73 | 0.80 | 0.80 | 0.57 | 0.33 | 0.13 | 0.00 |
| | | Byproduct edits | SD | 0.06 | 0.10 | 0.17 | 0.06 | 0.15 | 0.06 | 0.00 |
| | | % Indels | mean | 0.00 | 0.03 | 0.07 | 0.00 | 0.03 | 0.00 | 0.00 |
| | | | SD | 0.00 | 0.06 | 0.06 | 0.00 | 0.06 | 0.00 | 0.00 |
| | 1:1 | % Templated | mean | 50.25* | 49.33 | 43.27 | 30.03 | 16.77 | 6.87 | 3.50 |
| | | edits | SD | 4.74* | 1.96 | 2.01 | 6.40 | 6.40 | 3.19 | 1.21 |
| | | % Templated + | mean | 0.87 | 0.83 | 0.70 | 0.47 | 0.27 | 0.10 | 0.03 |
| | | Byproduct edits | SD | 0.06 | 0.06 | 0.10 | 0.15 | 0.15 | 0.10 | 0.06 |
| | | % Indels | mean | 0.00 | 0.07 | 0.07 | 0.00 | 0.00 | 0.00 | 0.00 |
| | | | SD | 0.00 | 0.06 | 0.06 | 0.00 | 0.00 | 0.00 | 0.00 |
| | 1:3 | % Templated | mean | 40.77 | 39.63 | 32.00 | 16.80 | 7.70 | 1.87 | 0.43 |
| | | edits | SD | 1.29 | 2.33 | 1.90 | 5.11 | 2.17 | 1.04 | 0.40 |
| | | % Templated + | mean | 0.80 | 0.57 | 0.50 | 0.20 | 0.10 | 0.00 | 0.00 |
| | | Byproduct edits | SD | 0.00 | 0.06 | 0.17 | 0.10 | 0.00 | 0.00 | 0.00 |
| | | % Indels | mean | 0.03 | 0.00 | 0.03 | 0.00 | 0.00 | 0.00 | 0.00 |
| | | | SD | 0.06 | 0.00 | 0.06 | 0.00 | 0.00 | 0.00 | 0.00 |

*N = 2

### Example 40—Templated Editing at the DNMT1 Locus in Primary Mouse Hepatocytes Using a Polymerase Delivered in Trans with a SpyCas9 Nickase at Different Ratios, Wherein Linkers with Different Lengths are Used to Link the Nickase and the Polymerase

This Example compared the editing efficiency of nCas9-polymerase constructs delivered in a split configuration and recombined by inteins, to that of a full-length nCas9-polymerase construct delivered as a single fusion protein. A SpyCas9 H840A nickase (nCas9) and a T5 polymerase variant were delivered into primary mouse hepatocytes (PMH) in cis (as a fusion protein) or in trans (using inteins), along with a tgRNA (G034987) comprising (i) a spacer sequence targeting the DNMT1 locus, (ii) a scaffold with the structure shown FIG. 6E, and (iii) a 3′ extension comprising a template encoding a G-to-C edit, and a DRS.

PMH (Gibco, Lot MC1007) were prepared as described in Example 1, and plated on a collagen I coated 96-well plate (Stem cell technologies, Cat. 100-0366) at a density of 15,000 cells/well. PMH were lipofected using Lipofectamine MessengerMAX (ThermoFisher Scientific, LMRNA015) lipoplexes, prepared according to manufacturer's instructions, with 25 nM/well of tgRNA and a 8-point 2-fold dilution series starting at 800 ng of mRNA. For each dose, the mRNA transfected consisted of:
a mRNA encoding a nCas9-T5(D164A, E166A) fusion protein (SEQ ID NO: 1304) comprising a nCas9 moiety, a 49-amino acid linker comprising a SV40 NLS, and a T5 polymerase variant moiety, or
a combination, in a 5:1 mass ratio or a 1:1 mass ratio, of either:
   a mRNA encoding nCas9-CfaN fusion protein (SEQ ID NO: 1362) comprising a nCas9 moiety and a CfaN split intein, and a mRNA encoding a CfaC-L30(SV40)-T5(D164A, E166A) fusion protein (SEQ ID NO: 1957) comprising a CfaC split intein capable of undergoing a trans-splicing reaction with a CfaN split intein, a 30-amino acid linker comprising a bpSV40 NLS, and a T5 polymerase variant moiety, or
   a mRNA encoding nCas9-CfaN (SEQ ID NO: 1362), and a mRNA encoding a CfaC-L49 (SV40)-T5(D164A, E166A) fusion protein (SEQ ID NO: 1918) comprising a CfaC split intein, a 49-amino acid linker comprising a SV40 NLS, and a T5 polymerase variant moiety.

The cells were lysed 72 hours post-treatment for NGS analysis as described in Example 1. Samples were run in triplicates.

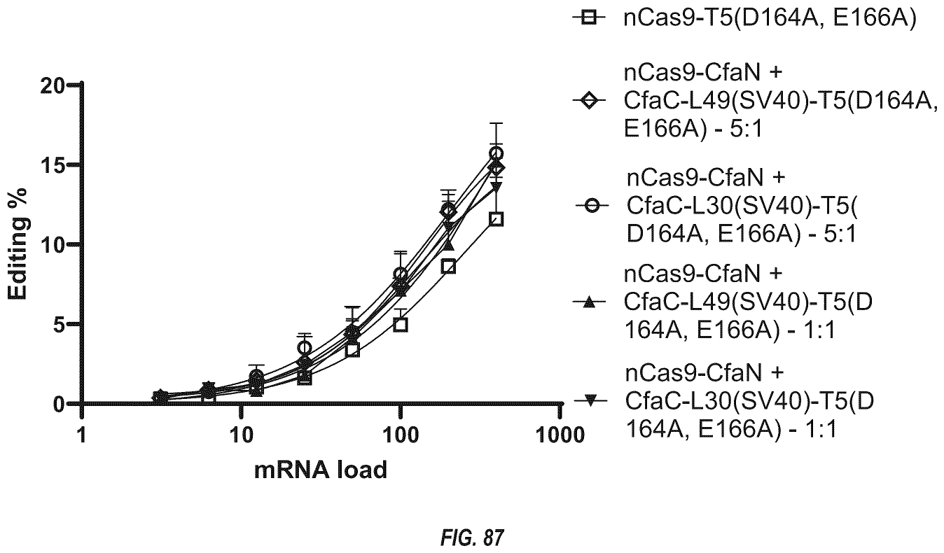

FIG. 87 and Table 90 show editing efficiencies obtained at the DNMT1 locus in PMH treated with a tgRNA (G034987), along with (i) nCas9-T5(D164A, E166A) (SEQ ID NO: 1304), (ii) a combination of nCas9-CfaN and CfaC-L30(SV40)-T5(D164A, E166A) (SEQ ID NO: 1957) in a 5:1 or 1:1 mass ratio, or (iii) a combination of nCas9-CfaN and CfaC-L49 (SV40)-T5(D164A, E166A) in a 5:1 or 1:1 mass ratio.

TABLE 90
Templated editing at the DNMT1 locus in PMH treated with a tgRNA (G034987), along
with (i) nCas9-T5(D164A, E166A), (ii) a combination of nCas9-CfaN and CfaC-L30(SV40)-
T5(D164A, E166A) in a 5:1 or 1:1 mass ratio, or (iii) a combination of nCas9-CfaN
and CfaC-L49(SV40)-T5(D164A, E166A) in a 5:1 or 1:1 mass ratio.

| | | | total mRNA (ng) | | | | | | | |
|---|---|---|---|---|---|---|---|---|---|---|
| | | | 800 | 400 | 200 | 100 | 50 | 25 | 12.5 | 6.25 |
| nCas9- | % Templated edits | mean | 11.60 | 8.62 | 4.97 | 3.39 | 1.63 | 1.05 | 0.32 | 0.29 |
| T5(D164A, | | SD | 2.63 | 0.48 | 0.98 | 0.86 | 0.39 | 0.16 | 0.19 | 0.17 |
| E166A) | % Templated + | mean | 0.09 | N/A | N/A | N/A | N/A | N/A | N/A | N/A |
| | byproduct edits | SD | 0.04 | N/A | N/A | N/A | N/A | N/A | N/A | N/A |
| | % Indels | mean | 0.01 | N/A | N/A | N/A | N/A | N/A | N/A | N/A |
| | | SD | 0.01 | N/A | N/A | N/A | N/A | N/A | N/A | N/A |
| nCas9-CfaN + | % Templated edits | mean | 15.71 | 12.19 | 8.13 | 4.50 | 3.51 | 1.72 | 0.77 | 0.37 |
| CfaC- | | SD | 1.90 | 0.95 | 1.44 | 0.85 | 0.90 | 0.18 | 0.54 | 0.17 |
| L30(SV40)- | % Templated + | mean | 0.15 | N/A | N/A | N/A | N/A | N/A | N/A | N/A |
| T5(D164A, | byproduct edits | SD | 0.02 | N/A | N/A | N/A | N/A | N/A | N/A | N/A |
| E166A) - 5:1 | % Indels | mean | 0.14 | N/A | N/A | N/A | N/A | N/A | N/A | N/A |
| | | SD | 0.12 | N/A | N/A | N/A | N/A | N/A | N/A | N/A |
| nCas9-CfaN + | % Templated edits | mean | 13.55 | 11.05 | 7.05 | 4.49 | 2.45 | 1.20 | 0.89 | 0.31 |
| CfaC- | | SD | 1.89 | 1.66 | 2.39 | 1.54 | 0.14 | 0.53 | 0.30 | 0.09 |
| L30(SV40)- | % Templated + | mean | 0.12 | N/A | N/A | N/A | N/A | N/A | N/A | N/A |
| T5(D164A, | byproduct edits | SD | 0.03 | N/A | N/A | N/A | N/A | N/A | N/A | N/A |
| E166A) - 1:1 | % Indels | mean | 0.12 | N/A | N/A | N/A | N/A | N/A | N/A | N/A |
| | | SD | 0.13 | N/A | N/A | N/A | N/A | N/A | N/A | N/A |
| nCas9-CfaN + | % Templated edits | mean | 14.83 | 12.04 | 7.38 | 4.34 | 2.60 | 1.46 | 0.87 | 0.38 |
| CfaC- | | SD | 0.17 | 1.39 | 0.51 | 1.77 | 1.63 | 0.98 | 0.08 | 0.13 |
| L49(SV40)- | % Templated + | mean | 0.18 | N/A | N/A | N/A | N/A | N/A | N/A | N/A |
| T5(D164A, | byproduct edits | SD | 0.01 | N/A | N/A | N/A | N/A | N/A | N/A | N/A |
| E166A) - 5:1 | % Indels | mean | 0.06 | N/A | N/A | N/A | N/A | N/A | N/A | N/A |
| | | SD | 0.06 | N/A | N/A | N/A | N/A | N/A | N/A | N/A |
| nCas9-CfaN + | % Templated edits | mean | 15.21 | 10.01 | 7.11 | 4.14 | 1.78 | 0.83 | 0.86 | 0.34 |
| CfaC- | | SD | 1.10 | 1.84 | 0.47 | 1.07 | 0.15 | 0.51 | 0.12 | 0.40 |
| L49(SV40)- | % Templated + | mean | 0.15 | N/A | N/A | N/A | N/A | N/A | N/A | N/A |
| T5(D164A, | byproduct edits | SD | 0.02 | N/A | N/A | N/A | N/A | N/A | N/A | N/A |
| E166A) - 1:1 | % Indels | mean | 0.09 | N/A | N/A | N/A | N/A | N/A | N/A | N/A |
| | | SD | 0.11 | N/A | N/A | N/A | N/A | N/A | N/A | N/A |

### Example 41—Templated Editing at the TTR Locus in Primary Cynomolgus Hepatocytes Using a First Portion of SpyCas9 Nickase Operably Linked to a First Intein and a Second Portion of SpyCas9 Operably Linked to a Second Intein

This Example compared the editing efficiency of nCas9-polymerase constructs delivered in a split configuration and recombined by inteins, to that of a full-length nCas9-polymerase construct delivered as a single fusion protein, wherein the split occurs in the interior of the nCas9 sequence. A first moiety of a SpyCas9 H840A nickase (Spy(2-713)) was delivered in trans with and a fusion protein comprising a second moiety of the a SpyCas9 H840A nickase (Spy(714-1368)) and a polymerase moiety (e.g., polymerase Kappa variant, or T5 polymerase variant) into primary cynomolgus hepatocytes (PCH), along with a mRNA encoding Vpx, and a tgRNA (G039268) comprising (i) a spacer sequence targeting the TTR locus, (ii) a scaffold with the structure shown in FIG. 6G, and (iii) a 3′ extension comprising a template encoding a TG-to-AC edit, resulting in a STOP codon, and a DRS.

PCH (Gibco, Lot CY423) were prepared as described in Example 1, and plated on a collagen I coated 96-well plate (Stem cell technologies, Cat. 100-0366) at a density of 40,000 cells/well. PCH were lipofected using pre-mixed lipid formulations with a molar composition of 50% Lipid A, 38% cholesterol, 9% DSPC, and 3% PEG2k-DMG as described in Example 1. Lipofections were performed with a tgRNA (G039268) at a fixed dose of 25 ng/well, and a mRNA load in an 8-point 2-fold dilution series starting at 300 ng mRNA.

The mRNA transfected consisted of:
a mRNA encoding a nCas9-PolKv2 fusion protein (SEQ ID NO: 1371) comprising a nCas9 moiety and a polymerase Kappa variant moiety,
a mRNA encoding a (nCas9-T5(D164A, E166A) fusion protein (SEQ ID NO: 1304) comprising a nCas9 moiety and a T5 polymerase variant moiety, or
a combination of either:
   a mRNA encoding a nCas9-CfaN fusion protein (SEQ ID NO: 1362) comprising a nCas9 moiety and a CfaN split intein, and a mRNA encoding a CfaC-PolK(1-526, GE411-412RV) fusion protein (SEQ ID NO: 1368) comprising a CfaC split intein capable of undergoing a trans-splicing reaction with a CfaN split intein, and a polymerase Kappa variant moiety, in a 5:1 mass ratio,
   a mRNA encoding a Spy(2-713)-CfaN fusion protein (SEQ ID NO: 1939) comprising a first moiety of a nCas9 sequence and a CfaN split intein, and a mRNA encoding a CfaC-Spy(714-1368)-PolKvariant fusion protein (SEQ ID NO: 1945) comprising a CfaC split intein, a second moiety of a nCas9 sequence, and a polymerase Kappa variant moiety, in a 1:1 mass ratio,
   a mRNA encoding a Spy(2-713)-CfaN fusion protein (SEQ ID NO: 1939), and a mRNA encoding a CfaC-Spy(714-1368)-T5polvariant fusion protein (SEQ ID NO: 1951) comprising a CfaC split intein, a second moiety of a nCas9 sequence, and a T5 polymerase variant moiety, in a 1:1 mass ratio, or
   a mRNA encoding a HMGB1dT-Spy(2-713)-CfaN fusion protein (SEQ ID NO: 1942) comprising an HMGB1 polypeptide comprising the HMGB1 Box A domain and Box B domain and lacking an acidic tail domain (“HMGB1_dTtail”), a first moiety of a nCas9 sequence, and a CfaN split intein, and a mRNA encoding a CfaC-Spy(714-1368)-PolKvariant fusion protein (SEQ ID NO: 1945) comprising a CfaC split intein, a second moiety of a nCas9 sequence, and a polymerase Kappa variant moiety, in a 1:1 mass ratio.

In addition, the cells were also treated with a LNP, prepared as described in Example 1 with a molar ratio of 50% Lipid A, 38% cholesterol, 9% DSPC, and 3% PEG2k-DMG and comprising a mRNA encoding Vpx (SEQ ID NO: 1462), at a fixed dose of 25 ng/well. LNPs were incubated in media containing 10% fetal bovine serum (FBS) at 37° C. for 10 minutes before being added to the cells.

Cells were incubated at 37° C., 5% CO₂ for 24 hours prior to treatment with the pre-mixed lipid formulations and with the LNPs. The cells were lysed 72 hours post-treatment for NGS analysis as described in Example 1. Samples were run in triplicates, unless otherwise indicated.

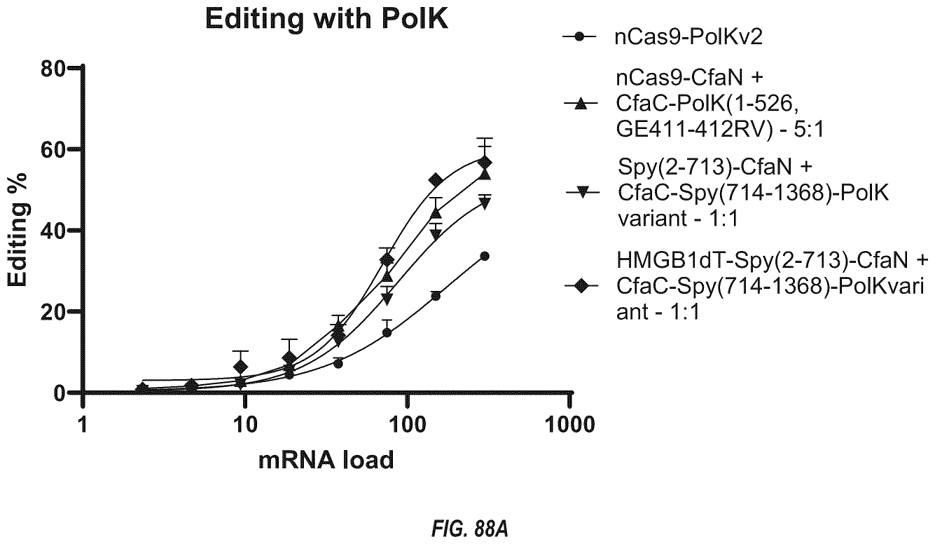

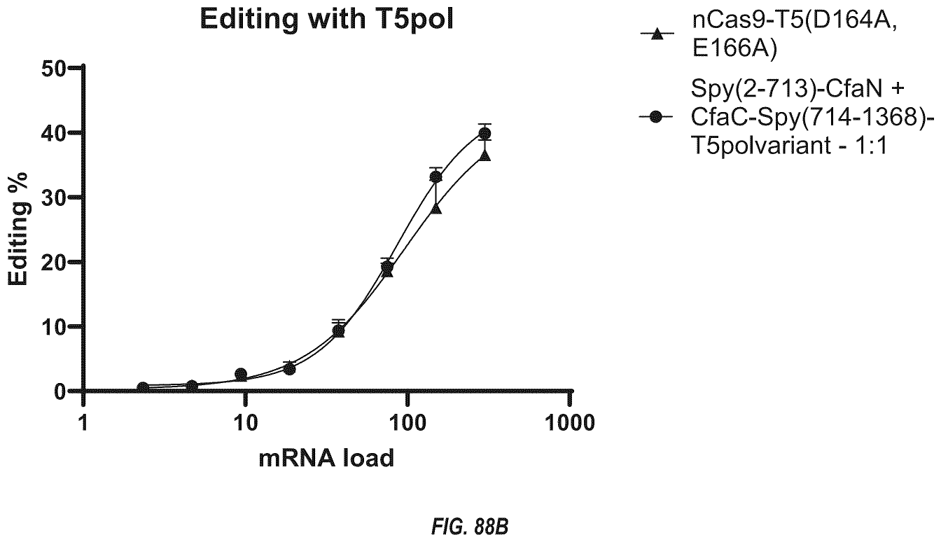

FIGS. 88A-B and Table 91 show editing efficiencies obtained at the TTR locus in PCH treated with a tgRNA (G039268), along with (i) nCas9-PolKv2, (ii) nCas9-T5(D164A, E166A, (iii) a combination of nCas9-CfaN and Cfa-PolK (1-526, GE411-412RV) at a 5:1 mass ratio, (iv) a combination of Spy(2-713)-CfaN with either one of CfaC-Spy(714-1368)-PolKvariant or CfaC-Spy(714-1368)-T5polvariant, at a 1:1 mass ratio, or (v) a combination of HMGB1dT-Spy(2-713)-CfaN and CfaC-Spy(714-1368)-PolKvariant at a 1:1 mass ratio, in the presence of Vpx.

TABLE 91
Editing at the TTR locus in PCH treated with a tgRNA (G039268), along with (i) nCas9-PolKv2, (ii)
nCas9-T5(D164A, E166A, (iii) a combination of nCas9-CfaN and Cfa-PolK(1-526, GE411-412RV) at a
5:1 mass ratio, (iv) a combination of Spy(2-713)-CfaN with either one of CfaC-Spy(714-1368)-PolKvariant
or CfaC-Spy(714-1368)-T5polvariant, at a 1:1 mass ratio, or (v) a combination of HMGB1dT-Spy(2-
713)-CfaN and CfaC-Spy(714-1368)-PolKvariant at a 1:1 mass ratio, in the presence of Vpx.

| | | | total mRNA (ng) | | | | | | | |
|---|---|---|---|---|---|---|---|---|---|---|
| | | | 300 | 150 | 75 | 37.5 | 18.75 | 9.38 | 4.69 | 2.34 |
| nCas9-PolKv2 | % Templated edits | mean | 33.69* | 23.78 | 14.80 | 7.13 | 4.41 | 2.38 | 1.34 | 0.24 |
| | | SD | 0.01* | 1.18 | 3.14 | 1.47 | 0.18 | 0.31 | 0.12 | 0.23 |
| | % Templated + | mean | 0.52* | N/A | N/A | N/A | N/A | N/A | N/A | N/A |
| | byproduct edits | SD | 0.13* | N/A | N/A | N/A | N/A | N/A | N/A | N/A |
| | % Indels | mean | 0.26* | N/A | N/A | N/A | N/A | N/A | N/A | N/A |
| | | SD | 0.13* | N/A | N/A | N/A | N/A | N/A | N/A | N/A |
| nCas9- | % Templated edits | mean | 54.07 | 44.51 | 28.89 | 16.62 | 6.87 | 2.99 | 1.67 | 1.06 |
| CfaN + CfaC- | | SD | 6.63 | 3.55 | 3.17 | 2.44 | 1.25 | 0.60 | 0.78 | 0.04 |
| PolK(1-526, | % Templated + | mean | 0.75 | N/A | N/A | N/A | N/A | N/A | N/A | N/A |
| GE411-412RV) - | byproduct edits | SD | 0.21 | N/A | N/A | N/A | N/A | N/A | N/A | N/A |
| 5:1 | % Indels | mean | 0.13 | N/A | N/A | N/A | N/A | N/A | N/A | N/A |
| | | SD | 0.10 | N/A | N/A | N/A | N/A | N/A | N/A | N/A |
| Spy(2-713)- | % Templated edits | mean | 46.49 | 38.70 | 22.93 | 12.63 | 5.37 | 2.04 | 0.87 | 0.44 |
| CfaN + CfaC- | | SD | 2.29 | 3.05 | 3.28 | 1.67 | 0.67 | 0.22 | 0.59 | 0.19 |
| Spy(714-1368)- | % Templated + | mean | 0.86 | N/A | N/A | N/A | N/A | N/A | N/A | N/A |
| PolKvariant - | byproduct edits | SD | 0.42 | N/A | N/A | N/A | N/A | N/A | N/A | N/A |
| 1:1 | % Indels | mean | 0.13 | N/A | N/A | N/A | N/A | N/A | N/A | N/A |
| | | SD | 0.10 | N/A | N/A | N/A | N/A | N/A | N/A | N/A |
| HMGB1dT- | % Templated edits | mean | 56.77 | 52.44 | 32.77 | 14.14 | 8.58 | 6.40 | 1.80 | 0.83 |
| Spy(2-713)- | | SD | 5.99 | 1.83 | 2.91 | 2.68 | 4.62 | 3.90 | 1.47 | 0.30 |
| CfaN +CfaC- | % Templated + | mean | 1.12 | N/A | N/A | N/A | N/A | N/A | N/A | N/A |
| Spy(714-1368)- | byproduct edits | SD | 0.30 | N/A | N/A | N/A | N/A | N/A | N/A | N/A |
| PolKvariant - | % Indels | mean | 0.30 | N/A | N/A | N/A | N/A | N/A | N/A | N/A |
| 1:1 | | SD | 0.20 | N/A | N/A | N/A | N/A | N/A | N/A | N/A |
| nCas9- | % Templated edits | mean | 36.56 | 28.39 | 18.60 | 9.24 | 3.87 | 2.41 | 0.88 | 0.33 |
| T5(D164A, | | SD | 2.30 | 4.26 | 1.21 | 1.82 | 0.44 | 0.92 | 0.41 | 0.23 |
| E166A) | % Templated + | mean | 0.48 | N/A | N/A | N/A | N/A | N/A | N/A | N/A |
| | byproduct edits | SD | 0.19 | N/A | N/A | N/A | N/A | N/A | N/A | N/A |
| | % Indels | mean | 0.31 | N/A | N/A | N/A | N/A | N/A | N/A | N/A |
| | | SD | 0.16 | N/A | N/A | N/A | N/A | N/A | N/A | N/A |
| Spy(2-713)- | % Templated edits | mean | 39.90 | 33.14 | 19.23 | 9.39 | 3.38 | 2.67 | 0.76 | 0.49 |
| CfaN + CfaC- | | SD | 1.44 | 1.44 | 1.35 | 1.22 | 1.13 | 0.58 | 0.28 | 0.26 |
| Spy(714-1368)- | % Templated + | mean | 0.38 | N/A | N/A | N/A | N/A | N/A | N/A | N/A |
| T5polvariant - | byproduct edits | SD | 0.24 | N/A | N/A | N/A | N/A | N/A | N/A | N/A |
| 1:1 | % Indels | mean | 0.20 | N/A | N/A | N/A | N/A | N/A | N/A | N/A |
| | | SD | 0.18 | N/A | N/A | N/A | N/A | N/A | N/A | N/A |

*N = 2

### Example 42—In Vivo Editing at the DNMT1 Locus in Mouse Liver Using a Polymerase Kappa Variant and a SpyCas9 Nickase Delivered in Trans or in Cis

This Example evaluated the in vivo editing efficiency of nCas9-polymerase constructs delivered in a split configuration and recombined by inteins in a mouse model. The editing efficiency at the DNMT1 locus in mouse liver was evaluated using a SpyCas9 H840A nickase (nCas9) and a polymerase Kappa variant delivered in cis (as a fusion protein) or in trans (using inteins), along with a tgRNA (G034987) comprising a spacer sequence targeting the DNMT1 locus and a 3′ extension encoding a G-to-C edit, in the presence of a ngRNA (G035727).

LNPs were prepared as described in Example 1, with a molar ratio of 50% Lipid A, 38% cholesterol, 9% DSPC, and 3% PEG2k-DMG, and formulated with a lipid amine to RNA phosphate (N:P) molar ratio of about 6. The LNPs comprised one of the following cargos:
a ngRNA (G035727),
a tgRNA (G034987) and an mRNA encoding nCas9-PolK (1-526, GE411-412RV) (SEQ ID NO: 1232) in a tgRNA:mRNA mass ratio of 1:2,
a tgRNA (G034987) and an mRNA encoding nCas9-PolKv2 (SEQ ID NO: 1371) in a tgRNA:mRNA mass ratio of 1:2, and
a tgRNA (G034987), an mRNA encoding a nCas9-CfaN fusion protein (SEQ ID NO: 1362) comprising a nCas9 moiety and a CfaN split intein, and an mRNA encoding a CfaC-PolK(1-526, GE411-412RV) fusion protein (SEQ ID NO: 1368) comprising a CfaC split intein capable of undergoing a trans-splicing reaction with a CfaN split intein, and a polymerase Kappa variant moiety, in a tgRNA: nCas9-CfaN: CfaC-PolK(1-526, GE411-412RV) mass ratio of 3:5:1.

Female C57BL/6J mice (Jackson Labs), six to seven weeks of age, were dosed with a LNP comprising ngRNA and a LNP comprising a tgRNA and mRNA as described above, based on individual body weight measured the morning of dosing. LNPs were dosed via the lateral tail vein at a dose of 3.5 mpk of total RNA (0.5 mpk of ngRNA, 1 mpk of tgRNA, and 2 mpk of mRNA). The number of animals in each group was N=4. The animals were periodically observed for adverse effects for at least 24 hours post dose.

Animals were euthanized seven days post-dose by exsanguination via cardiac puncture under isoflurane anesthesia. Liver tissue was collected, and genomic DNA was isolated from the liver tissue for editing measurement by NGS, as described in Example 1.

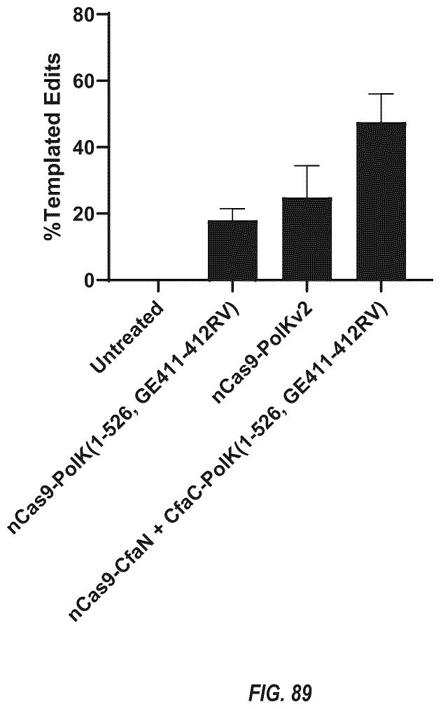

Table 92 and FIG. 89 show editing efficiencies at the DNMT1 locus in mice treated with the LNPs as described above.

TABLE 92
Editing efficiencies at the DNMTI locus in mice treated
with a fusion protein comprising a nCas9 moiety and a polymerase
Kappa variant moiety, or a combination of a nCas9-CfaN
fusion protein and a CfaC-PolK(1-526, GE411-412RV), along
with a tgRNA (G034987), and a ngRNA (G035727).

| | % Templated | | % Templated + | | | |
|---|---|---|---|---|---|---|
| | Edits | | Byproduct Edits | | % Indels | |
| mRNA | Mean | SD | Mean | SD | Mean | SD |
| Untreated | 0.00 | 0.00 | 0.00 | 0.00 | 0.00 | 0.01 |
| nCas9-PolK(1-526, | 18.05 | 3.46 | 0.64 | 0.16 | 0.42 | 0.12 |
| GE411-412RV) | | | | | | |
| nCas9-PolKv2 | 24.90 | 9.53 | 0.90 | 0.43 | 0.62 | 0.39 |
| nCas9-CfaN + | 47.58 | 8.44 | 2.01 | 0.41 | 1.32 | 0.19 |
| CfaC-PolK (1-526, | | | | | | |
| GE411-412RV) | | | | | | |

### Example 43—In Vivo Editing at the DNMT1 Locus in Mouse Liver Using a Polymerase Kappa Variant and a SpyCas9 Nickase Delivered in Cis at Different Doses

This Example evaluated the in vivo editing efficiency of nCas9-polymerase constructs delivered at different doses in a split configuration and recombined by inteins in a mouse model. The editing efficiency at the DNMT1 locus in mouse liver was evaluated using a SpyCas9 H840A nickase (nCas9) and a polymerase Kappa variant delivered in trans (using inteins), along with a tgRNA (G034987) comprising a spacer sequence targeting the DNMT1 locus and a 3′ extension encoding a G-to-C edit, in the presence of a ngRNA (G035727).

LNPs were prepared as described in Example 1, with a molar ratio of 50% Lipid B, 38% cholesterol, 9% DSPC, and 3% C13 Ether PEG lipid, and formulated with a lipid amine to RNA phosphate (N:P) molar ratio of about 6. The LNPs comprised a ngRNA (G035727), a tgRNA (G034987), an mRNA encoding a nCas9-CfaN fusion protein (SEQ ID NO: 1362) comprising a nCas9 moiety and a CfaN split intein, and an mRNA encoding a CfaC-PolK(1-526, GE411-412RV) fusion protein (SEQ ID NO: 1368) comprising a CfaC split intein capable of undergoing a trans-splicing reaction with a CfaN split intein, and a polymerase Kappa variant moiety. The mass ratios of the different RNA components comprised in the LNPs were 3:2 tgRNA:ngRNA, 5:1 nCas9-CfaN: CfaC-PolK(1-526, GE411-412RV), and 1:2 gRNA: mRNA.

Female C57BL/6J mice (Jackson Labs), six to seven weeks of age, were dosed with the LNPs as described above, based on individual body weight measured the morning of dosing. LNPs were dosed via the lateral tail vein at a dose of 0.1, 0.3 and 1 mpk of total RNA. The number of animals in each group was N=5. The animals were periodically observed for adverse effects for at least 24 hours post dose.

Animals were euthanized six days post-dose by exsanguination via cardiac puncture under isoflurane anesthesia. Liver tissue was collected, and genomic DNA was isolated from the liver tissue for editing measurement by NGS, as described in Example 1.

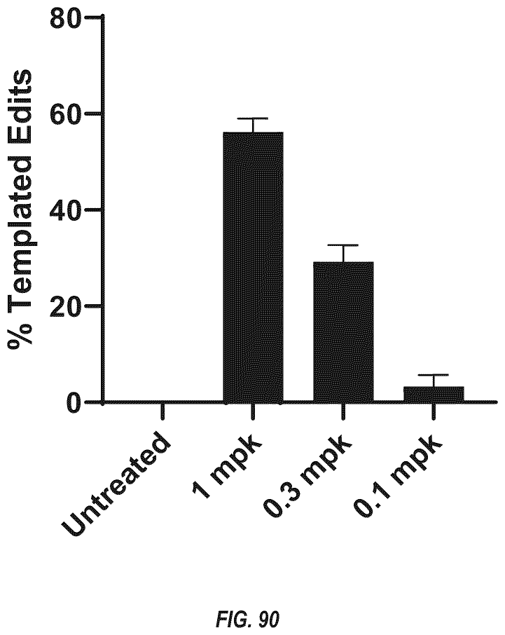

Table 93 and FIG. 90 show editing efficiencies at the DNMT1 locus in mice treated with the LNPs as described above.

TABLE 93
Editing efficiencies at the DNMTI locus in mice treated
with a combination of a nCas9-CfaN fusion protein
and a CfaC-PolK(1-526, GE411-412RV), along with
a tgRNA (G034987), and a ngRNA (G035727).

| | % Templated | | % Templated + | | | |
|---|---|---|---|---|---|---|
| Dose | Edits | | Byproduct Edits | | % Indels | |
| (mpk) | Mean | SD | Mean | SD | Mean | SD |
| Untreated | 0.00 | 0.01 | 0.00 | 0.00 | 0.00 | 0.00 |
| 1 | 56.23 | 2.83 | 1.41 | 0.09 | 0.33 | 0.15 |
| 0.3 | 29.24 | 3.47 | 0.60 | 0.09 | 0.05 | 0.03 |
| 0.1 | 3.33 | 2.36 | 0.06 | 0.04 | 0.01 | 0.00 |

### Example 44—Editing at the VEGFA Locus in PHH Using Constructs Comprising a SpyCas9 Nickase, a Polymerase Kappa Variant and Editing Enhancers Operably Linked to Split Inteins

This experiment compares the editing efficiency of a SpyCas9 H840A nickase (nCas9) delivered in trans with a polymerase in the presence of editing enhancers, wherein the editing enhancers are delivered (i) in trans, or (ii) in cis with either the polymerase or the nCas9.

Primary human hepatocytes (PHH, Gibco, Lot 8381) were prepared as described in Example 1, and plated on a collagen I coated 96-well plate (Stem cell technologies, Cat. 100-0366) at a density of 33,000 cells/well. LNPs were prepared as described in Example 1 to deliver RNA cargo to the cells. The LNPs were made with a molar ratio of 50% Lipid A, 38% cholesterol, 9% DSPC, and 3% PEG2k-DMG, and formulated with a lipid amine to RNA phosphate (N:P) molar ratio of about 6.

The cells were treated with a total RNA cargo comprising a tgRNA cargo and a mRNA cargo. The tgRNA cargo, delivered via a LNP, comprised a tgRNA (G032596). The tgRNA comprised a spacer sequence targeting the VEGFA locus, a scaffold with the structure shown FIG. 6E, and a 3′ extension comprising a template encoding a GAGG-to-CACT edit, and a DRS. The mRNA cargo consisted of one of the following cargos:
a LNP comprising a mRNA encoding nCas9-PolKv2 (SEQ ID NO: 1371),
a LNP comprising a mRNA encoding nCas9-PolKv2 (SEQ ID NO: 1371), a LNP comprising a mRNA encoding Vpx (SEQ ID NO: 1462), and a LNP comprising a mRNA encoding FEN1 (SEQ ID NO: 1465), wherein the mass ratio of nCas9-PolKv2:Vpx:FEN1 was 12:1:1,
a LNP comprising a mRNA encoding a nCas9-CfaN fusion protein (SEQ ID NO: 1362) comprising a nCas9 moiety and a CfaN split intein, and a LNP comprising a mRNA encoding a CfaC-polKvariant-FEN1 fusion protein (SEQ ID NO: 1283), comprising a CfaC split intein, a polymerase Kappa variant moiety, and a FEN1 moiety, wherein the LNPs were delivered in a nCas9:polymerase mass ratio of 5:1,
a LNP comprising a mRNA encoding a nCas9-CfaN fusion protein (SEQ ID NO: 1362), a LNP comprising a mRNA encoding a CfaC-polKvariant-FEN1 fusion protein (SEQ ID NO: 1283), and a LNP comprising a mRNA encoding Vpx (SEQ ID NO: 1462), wherein the LNPs were delivered in a nCas9:polymerase:Vpx mass ratio of 5:1:1.
a LNP comprising a mRNA encoding a nCas9-CfaN fusion protein (SEQ ID NO: 1362), and an LNP comprising a mRNA encoding a CfaC-polKvariant-FEN1-P2A-Vpx fusion protein (SEQ ID NO: 1286), comprising a CfaC split intein, a polymerase Kappa variant moiety, a FEN1 moiety, a P2A peptide, and a Vpx moiety, wherein the LNPs were delivered in a nCas9:polymerase mass ratio of 5:1,
a LNP comprising a mRNA encoding a nCas9-CfaN fusion protein (SEQ ID NO: 1362), and an LNP comprising a mRNA encoding a CfaC-polKvariant-T2A-FEN1-P2A-Vpx fusion protein (SEQ ID NO: 1289), comprising a CfaC split intein, a polymerase Kappa variant moiety, a T2A peptide, a FEN1 moiety, a P2A peptide, and a Vpx moiety, wherein the LNPs were delivered in a nCas9:polymerase mass ratio of 5:1,
a LNP comprising a mRNA encoding a nCas9-CfaN fusion protein (SEQ ID NO: 1362), and an LNP comprising a mRNA encoding a CfaC-polKvariant-T2A-Vpx-P2A-FEN1 fusion protein (SEQ ID NO: 1292), comprising a CfaC split intein, a polymerase Kappa variant moiety, a T2A domain, a Vpx moiety, a P2A peptide, and a FEN1 moiety, wherein the LNPs were delivered in a nCas9:polymerase mass ratio of 5:1,
a LNP comprising a mRNA encoding a nCas9-CfaN fusion protein (SEQ ID NO: 1362), and a LNP comprising a mRNA encoding a CfaC-polKvariant-EI-T2A-FEN1-KI-P2A-Vpx fusion protein (SEQ ID NO: 1295), comprising a CfaC split intein, a polymerase Kappa variant moiety, a EI domain, a T2A domain, a FEN1 moiety, a KI domain, a P2A peptide, and a Vpx moiety, wherein the LNPs were delivered in a nCas9:polymerase mass ratio of 5:1,
a LNP comprising a mRNA encoding a nCas9-CfaN-T2A-Vpx fusion protein (SEQ ID NO: 1277) comprising a nCas9 moiety, a CfaN split intein, a T2A domain, and a Vpx moiety, and a LNP comprising a mRNA encoding a CfaC-polKvariant-FEN1 fusion protein (SEQ ID NO: 1283), wherein the LNPs were delivered in a nCas9:polymerase mass ratio of 5:1, or
a LNP comprising a mRNA encoding a HMGBldT-nCas9-CfaN-T2A-Vpx fusion protein (SEQ ID NO: 1280) comprising HMGB1_dTail, a nCas9 moiety, a CfaN split intein, a T2A domain, and a Vpx moiety, and a LNP comprising a mRNA encoding a CfaC-polKvariant-FEN1 fusion protein (SEQ ID NO: 1283), wherein the LNPs were delivered in a nCas9:polymerase mass ratio of 5:1.

Cells were incubated at 37° C., 5% CO₂ for 24 hours prior to treatment with LNPs. LNPs were incubated in media containing 10% fetal bovine serum (FBS) at 37° C. for 10 minutes. Post-incubation, the LNPs were delivered to the cells, in an 8-point 3-fold dilution series starting at 300 ng total RNA cargo, wherein the mass ratio of tgRNA to mRNA encoding nCas9 and polymerase was kept constant at 1:2. The cells were lysed 72 hours post-treatment for NGS analysis as described in Example 1. Samples were run in duplicates, unless otherwise indicated.

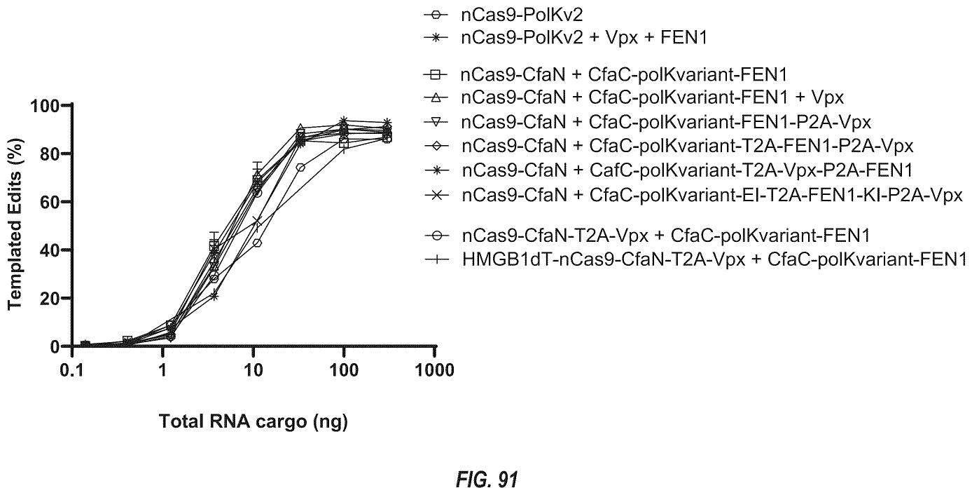

FIG. 91 and Table 94 show editing efficiencies obtained at the VEGFA locus in PHH treated with a tgRNA (G032596), along with split intein constructs comprising a nCas9 and a polymerase delivered in trans, with editing enhancers delivered in trans or in cis with the split intein constructs.

TABLE 94
Editing efficiencies obtained at the VEGFA locus in PHH treated with a tgRNA (G032596),
along with split intein constructs comprising a nCas9 and a polymerase delivered in trans,
with editing enhancers delivered in trans or in cis with the split intein constructs.

| | | | Total RNA cargo (ng) | | | | | | | |
|---|---|---|---|---|---|---|---|---|---|---|
| mRNA species | | | 300 | 100 | 33.33 | 11.11 | 3.7 | 1.23 | 0.41 | 0.14 |
| nCas9-PolKv2 | % Templated | Mean | 85.93* | 86.10 | 74.28 | 42.94* | 28.02 | 8.79 | 1.32* | 0.59 |
| | Edits | SD | N/A | 0.33 | 5.03 | N/A | 5.47 | 1.37 | N/A | 0.43 |

| | | | | | | | | | |
|---|---|---|---|---|---|---|---|---|---|
| % Templated | Mean | 1.93* | 1.90 | N/A | N/A | 0.40* | 0.14* | 0.01* | 0.01* |
| Edits + | SD | N/A | 0.21 | N/A | N/A | N/A | N/A | N/A | N/A |
| Byproducts | | | | | | | | | |
| % Indels | Mean | 0.78 | 0.70 | N/A | N/A | 0.13* | 0.03* | 0.01* | 0.00* |
| | SD | 0.00 | 0.05 | N/A | N/A | N/A | N/A | N/A | N/A |

| | | | | | | | | | | |
|---|---|---|---|---|---|---|---|---|---|---|
| nCas9-PolKv2 + | % Templated | Mean | 92.91 | 93.65 | 84.04* | N/A | 20.80* | 7.55* | 2.09 | 0.78 |
| Vpx + FEN1 | Edits | SD | 0.24 | 0.27 | N/A | N/A | N/A | N/A | 0.06 | 0.23 |

| | | | | | | | | | |
|---|---|---|---|---|---|---|---|---|---|
| % Templated Edits + | Mean | 2.31 | 2.15 | 1.73* | N/A | 0.50* | 0.17* | 0.05 | 0.11 |
| Byproducts | SD | 0.05 | 0.10 | N/A | N/A | N/A | N/A | 0.02 | 0.13 |
| % Indels | Mean | 0.79 | 0.66 | 0.39* | N/A | 0.21* | 0.06* | 0.03 | 0.00 |

| | | SD | 0.06 | 0.01 | N/A | N/A | N/A | N/A | 0.02 | 0.00 |
|---|---|---|---|---|---|---|---|---|---|---|
| nCas9-CfaN + CfaC- | % Templated | Mean | 86.82* | 84.40* | 85.36* | 69.04 | 41.66 | 8.73* | 2.34* | 0.18* |
| polKvariant-FEN1 | Edits | SD | N/A | N/A | N/A | 4.53 | 5.71 | N/A | N/A | N/A |

| | | | | | | | | | |
|---|---|---|---|---|---|---|---|---|---|
| % Templated | Mean | 2.01* | 1.49* | 1.64* | 1.17 | 0.71 | 0.12* | 0.08* | 0.00* |
| Edits + | SD | N/A | N/A | N/A | 0.05 | 0.09 | N/A | N/A | N/A |
| Byproducts | | | | | | | | | |
| % Indels | Mean | 0.95* | 0.76* | 0.65* | 0.31 | 0.17 | 0.03* | 0.03* | 0.00* |
| | SD | N/A | N/A | N/A | 0.01 | 0.02 | N/A | N/A | N/A |

| | | | | | | | | | | |
|---|---|---|---|---|---|---|---|---|---|---|
| nCas9-CfaN + CfaC- | % Templated | Mean | 90.43 | 91.95* | 90.65* | 71.37 | 33.48* | 5.18* | 1.26 | 0.21 |
| polKvariant- | Edits | SD | 0.90 | N/A | N/A | 5.11 | N/A | N/A | 0.47 | 0.11 |
| FEN1 + Vpx | | | | | | | | | | |

| | | | | | | | | | |
|---|---|---|---|---|---|---|---|---|---|
| % Templated Edits + | Mean | 2.53 | 2.13* | 2.24* | 1.51 | 0.63* | 0.17* | 0.02 | 0.00 |
| Byproducts | SD | 0.07 | N/A | N/A | 0.01 | N/A | N/A | 0.01 | 0.00 |
| % Indels | Mean | 1.03 | 0.68* | 0.72* | 0.34 | 0.10* | 0.00* | 0.00 | 0.01 |

| | | SD | 0.28 | N/A | N/A | 0.04 | N/A | N/A | 0.00 | 0.00 |
|---|---|---|---|---|---|---|---|---|---|---|
| nCas9-CfaN + CfaC- | % Templated | Mean | 88.80 | 90.20 | 88.22* | 64.50 | 32.95 | 3.56 | 0.88* | 0.07 |
| polKvariant-FEN1- | Edits | SD | 0.33 | 0.03 | N/A | 4.12 | 5.95 | 0.49 | N/A | 0.01 |
| P2A-Vpx | | | | | | | | | | |

| | | | | | | | | | |
|---|---|---|---|---|---|---|---|---|---|
| % Templated Edits + | Mean | 1.89 | 1.89 | 1.62* | 1.05 | 0.47 | 0.09 | 0.03* | 0.00 |
| Byproducts | SD | 0.09 | 0.07 | N/A | 0.21 | 0.04 | 0.03 | N/A | 0.00 |
| % Indels | Mean | 0.80 | 0.77 | 0.63* | 0.31 | 0.15 | 0.02 | 0.02* | 0.01 |

| | | SD | 0.10 | 0.05 | N/A | 0.06 | 0.05 | 0.01 | N/A | 0.01 |
|---|---|---|---|---|---|---|---|---|---|---|
| nCas9-CfaN + CfaC- | % Templated | Mean | 91.27* | 89.96* | 85.43 | 65.86 | 35.37 | 4.83* | 1.07* | 0.06 |
| polKvariant-T2A- | Edits | SD | N/A | N/A | 1.15 | 2.89 | 6.46 | N/A | N/A | 0.01 |
| FEN1-P2A-Vpx | | | | | | | | | | |

| | | | | | | | | | |
|---|---|---|---|---|---|---|---|---|---|
| % Templated | Mean | 1.68* | 1.67* | 1.60 | 1.15 | 0.50 | 0.09* | 0.02* | 0.00 |
| Edits + | SD | N/A | N/A | 0.12 | 0.16 | 0.13 | N/A | N/A | 0.00 |
| Byproducts | | | | | | | | | |
| % Indels | Mean | 0.65* | 0.64* | 0.45 | 0.33 | 0.13 | 0.03* | 0.00* | 0.01 |
| | SD | N/A | N/A | 0.03 | 0.07 | 0.01 | N/A | N/A | 0.01 |

| | | | | | | | | | | |
|---|---|---|---|---|---|---|---|---|---|---|
| nCas9-CfaN + CfaN- | % Templated | Mean | 88.86 | 88.23* | 85.27 | 68.18 | 38.53 | 7.70* | 0.97 | 0.13 |
| polKvariant-T2A-Vpx- | Edits | SD | 0.03 | N/A | 0.32 | 0.21 | 5.07 | N/A | 0.25 | 0.04 |
| P2A-FEN1 | | | | | | | | | | |

| | | | | | | | | | |
|---|---|---|---|---|---|---|---|---|---|
| % Templated Edits + | Mean | 1.64 | 1.60* | 1.42 | 1.23 | 0.63 | 0.15* | 0.02 | 0.00 |
| Byproducts | SD | 0.00 | N/A | 0.05 | 0.06 | 0.06 | N/A | 0.01 | 0.00 |
| % Indels | Mean | 0.65 | 0.53* | 0.46 | 0.32 | 0.15 | 0.01* | 0.02 | 0.01 |

| | | SD | 0.04 | N/A | 0.01 | 0.06 | 0.00 | N/A | 0.01 | 0.01 |
|---|---|---|---|---|---|---|---|---|---|---|
| nCas9-CfaN + CfaC- | % Templated | Mean | 89.55* | 90.26* | 86.53 | 52.11* | 40.41 | 5.88 | 0.28* | 0.10 |
| polKvariant-EI-T2A- | Edits | SD | N/A | N/A | 0.15 | N/A | 3.92 | 1.85 | N/A | 0.02 |
| FEN1-KI-P2A-Vpx | | | | | | | | | | |

| | | | | | | | | | |
|---|---|---|---|---|---|---|---|---|---|
| % Templated Edits + | Mean | 1.96* | 2.01* | 1.57 | 1.88* | 0.66 | 0.08 | 0.00* | 0.00 |
| Byproducts | SD | N/A | N/A | 0.01 | N/A | 0.11 | 0.03 | N/A | 0.00 |
| % Indels | Mean | 0.75* | 0.70* | 0.50 | N/A | 0.22 | 0.05 | 0.00* | 0.01 |

| | | SD | N/A | N/A | 0.01 | N/A | 0.02 | 0.02 | N/A | 0.01 |
|---|---|---|---|---|---|---|---|---|---|---|
| nCas9-CfaN-T2A- | % Templated | Mean | 88.36* | 88.49* | 86.77* | 63.76* | 29.42* | 4.17* | 0.58* | N/A |
| Vpx + CfaC- | Edits | SD | N/A | N/A | N/A | N/A | N/A | N/A | N/A | N/A |
| polKvariant-FEN1 | | | | | | | | | | |

| | | | | | | | | | |
|---|---|---|---|---|---|---|---|---|---|
| % Templated | Mean | 2.25* | 2.11* | 1.54* | 1.26* | 0.44* | 0.00* | 0.00* | N/A |
| Edits + | SD | N/A | N/A | N/A | N/A | N/A | N/A | N/A | N/A |
| Byproducts | | | | | | | | | |
| % Indels | Mean | 0.69* | 0.72* | 0.62* | 0.27* | 0.19* | 0.18* | 0.00* | N/A |
| | SD | N/A | N/A | N/A | N/A | N/A | N/A | N/A | N/A |

| | | | | | | | | | | |
|---|---|---|---|---|---|---|---|---|---|---|
| HMGB1dT-nCas9-CfaN- | % Templated | Mean | 86.31* | 81.89 | N/A | 49.24* | 22.15* | N/A | 0.27* | N/A |
| T2A-Vpx + CfaC- | Edits | SD | N/A | 0.71 | N/A | N/A | N/A | N/A | N/A | N/A |
| polKvariant-FEN1 | | | | | | | | | | |

| | | | | | | | | | |
|---|---|---|---|---|---|---|---|---|---|
| % Templated | Mean | 1.71* | 1.55 | N/A | 0.82* | 0.42* | N/A | 0.00* | N/A |
| Edits + | SD | N/A | 0.12 | N/A | N/A | N/A | N/A | N/A | N/A |
| Byproducts | Mean | 1.19* | 0.89 | N/A | 0.38* | 0.45* | N/A | 0.01* | N/A |
| % Indels | SD | N/A | 0.04 | N/A | N/A | N/A | N/A | N/A | N/A |

*N = 1

### Example 45—Editing at the TTR Locus in PCH Using Constructs Comprising a SpyCas9 Nickase, a Polymerase Kappa Variant and Editing Enhancers Operably Linked to Split Inteins

This Example evaluates the editing efficiency of a SpyCas9 H840A nickase (nCas9) delivered in trans with a polymerase in the presence of editing enhancers, wherein the editing enhancers are delivered (i) in trans, or (ii) in cis with either the polymerase or the nCas9.

Primary cynomolgus hepatocytes (PCH, Gibco, Lot CY423) were prepared as described in Example 1, and plated on a collagen I coated 96-well plate (Stem cell technologies, Cat. 100-0366) at a density of 40,000 cells/well. LNPs were prepared as described in Example 1 to deliver RNA cargo to the cells. The LNPs were made with a molar ratio of 50% Lipid A, 38% cholesterol, 9% DSPC, and 3% PEG2k-DMG, and formulated with a lipid amine to RNA phosphate (N:P) molar ratio of about 6.

The cells were treated with a total RNA cargo comprising a tgRNA cargo, a ngRNA cargo and a mRNA cargo. The tgRNA cargo, delivered via a LNP, comprised a tgRNA (G039267; SEQ ID NO: 597). The tgRNA comprised a spacer sequence targeting the TTR locus, a scaffold with the structure shown FIG. 6C, and a 3′ extension comprising a template encoding a TG-to-AC edit, and a DRS. The ngRNA cargo, delivered via a LNP, comprised a ngRNA (G032630; SEQ ID NO: 599). The mRNA cargo consisted of one of the following cargos:
a LNP comprising a mRNA encoding a nCas9-CfaN fusion protein (SEQ ID NO: 1362) comprising a nCas9 moiety and a CfaN split intein, and a mRNA encoding a encoding a CfaC-PolK (1-526, GE411-412RV) fusion protein (SEQ ID NO: 1368) comprising a CfaC split intein capable of undergoing a trans-splicing reaction with a CfaN split intein and a PolK variant moiety, wherein the nCas9:polymerase mass ratio was 5:1,
a LNP comprising a mRNA encoding a nCas9-CfaN fusion protein (SEQ ID NO: 1362), and a mRNA encoding a CfaC-PolK(1-526, GE411-412RV) fusion protein (SEQ ID NO: 1368), wherein the nCas9:polymerase mass ratio was 5:1, and a LNP comprising a mRNA encoding Vpx (SEQ ID NO: 1462), wherein the LNPs were delivered in a Cas9: polymerase: Vpx mass ratio of 5:1:1,
a LNP comprising a mRNA encoding a nCas9-CfaN fusion protein (SEQ ID NO: 1362), and a mRNA encoding a encoding a CfaC-PolK(1-526, GE411-412RV) fusion protein (SEQ ID NO: 1368), wherein the nCas9:polymerase mass ratio was 5:1, a LNP comprising a mRNA encoding Vpx (SEQ ID NO: 1462), and a LNP comprising a mRNA encoding FEN1 (SEQ ID NO: 1465), wherein the LNPs were delivered in a nCas9:polymerase:Vpx:FEN mass ratio of 10:2:1:1,
a LNP comprising a mRNA encoding a nCas9-CfaN fusion protein (SEQ ID NO: 1362), and a LNP comprising a mRNA encoding a CfaC-polKvariant-FEN1 fusion protein (SEQ ID NO: 1283), comprising a CfaC split intein, a polymerase Kappa variant moiety, and a FEN1 moiety, wherein the LNPs were delivered in a nCas9:polymerase mass ratio of 5:1,
a LNP comprising a mRNA encoding a nCas9-CfaN fusion protein (SEQ ID NO: 1362), a LNP comprising a mRNA encoding a CfaC-polKvariant-FEN1 fusion protein (SEQ ID NO: 1283), and a LNP comprising a mRNA encoding Vpx (SEQ ID NO: 1462), wherein the LNPs were delivered in a nCas9:polymerase:Vpx mass ratio of 5:1:1,
a LNP comprising a mRNA encoding a nCas9-CfaN fusion protein (SEQ ID NO: 1362), and an LNP comprising a mRNA encoding a CfaC-polKvariant-FEN1-P2A-Vpx fusion protein (SEQ ID NO: 1286), comprising a CfaC split intein, a polymerase Kappa variant moiety, a FEN1 moiety, a P2A peptide, and a Vpx moiety, wherein the LNPs were delivered in a nCas9:polymerase mass ratio of 5:1,
a LNP comprising a mRNA encoding a nCas9-CfaN fusion protein (SEQ ID NO: 1362), and an LNP comprising a mRNA encoding a CfaC-polKvariant-T2A-FEN1-P2A-Vpx fusion protein (SEQ ID NO: 1289), comprising a CfaC split intein, a polymerase Kappa variant moiety, a T2A peptide, a FEN1 moiety, a P2A peptide, and a Vpx moiety, wherein the LNPs were delivered in a nCas9:polymerase mass ratio of 5:1,
a LNP comprising a mRNA encoding a nCas9-CfaN fusion protein (SEQ ID NO: 1362), and an LNP comprising a mRNA encoding a CfaC-polKvariant-T2A-Vpx-P2A-FEN1 fusion protein (SEQ ID NO: 1292), comprising a CfaC split intein, a polymerase Kappa variant moiety, a T2A peptide, a Vpx moiety, a P2A peptide, and a FEN1 moiety, wherein the LNPs were delivered in a nCas9:polymerase mass ratio of 5:1,
a LNP comprising a mRNA encoding a nCas9-CfaN fusion protein (SEQ ID NO: 1362), and a LNP comprising a mRNA encoding a CfaC-polKvariant-EI-T2A-FEN1-KI-P2A-Vpx fusion protein (SEQ ID NO: 1295), comprising a CfaC split intein, a polymerase Kappa variant moiety, a EI domain, a T2A peptide, a FEN1 moiety, a KI domain, a P2A peptide, and a Vpx moiety, wherein the LNPs were delivered in a nCas9:polymerase mass ratio of 5:1,
a LNP comprising a mRNA encoding a nCas9-CfaN-T2A-Vpx fusion protein (SEQ ID NO: 1277) comprising a nCas9 moiety, a CfaN split intein, a T2A peptide, and a Vpx moiety, and a LNP comprising a mRNA encoding a CfaC-polKvariant-FEN1 fusion protein (SEQ ID NO: 1283), wherein the LNPs were delivered in a nCas9:polymerase mass ratio of 5:1,
a LNP comprising a mRNA encoding a HMGB1dT-nCas9-CfaN fusion protein (SEQ ID NO: 1365) comprising a HMGB1_dTail, a nCas9 moiety and a CfaN split intein, and a LNP comprising a mRNA encoding a CfaC-polKvariant-FEN1 fusion protein (SEQ ID NO: 1283), wherein the LNPs were delivered in a nCas9:polymerase mass ratio of 5:1, or
a LNP comprising a mRNA encoding a HMGBldT-nCas9-CfaN-T2A-Vpx fusion protein (SEQ ID NO: 1280) comprising HMGB1_dTail, a nCas9 moiety, a CfaN split intein, a T2A peptide, and a Vpx moiety, and a LNP comprising a mRNA encoding a CfaC-polKvariant-FEN1 fusion protein (SEQ ID NO: 1283), wherein the LNPs were delivered in a nCas9:polymerase mass ratio of 5:1.

Cells were incubated at 37° C., 5% CO₂ for 24 hours prior to treatment with LNPs. LNPs were incubated in media containing 10% fetal bovine serum (FBS) at 37° C. for 10 minutes. Post-incubation, the LNPs were delivered to the cells, in an 8-point 3-fold dilution series starting at 300 ng total RNA cargo, wherein the mass ratio of tgRNA to ngRNA was kept constant at 4:1 and the mass ratio of gRNA (i.e., tgRNA and ngRNA) to mRNA encoding nCas9 and polymerase was kept constant at 1:2. The cells were lysed 72 hours post-treatment for NGS analysis as described in Example 1. Samples were run in triplicates, unless otherwise indicated.

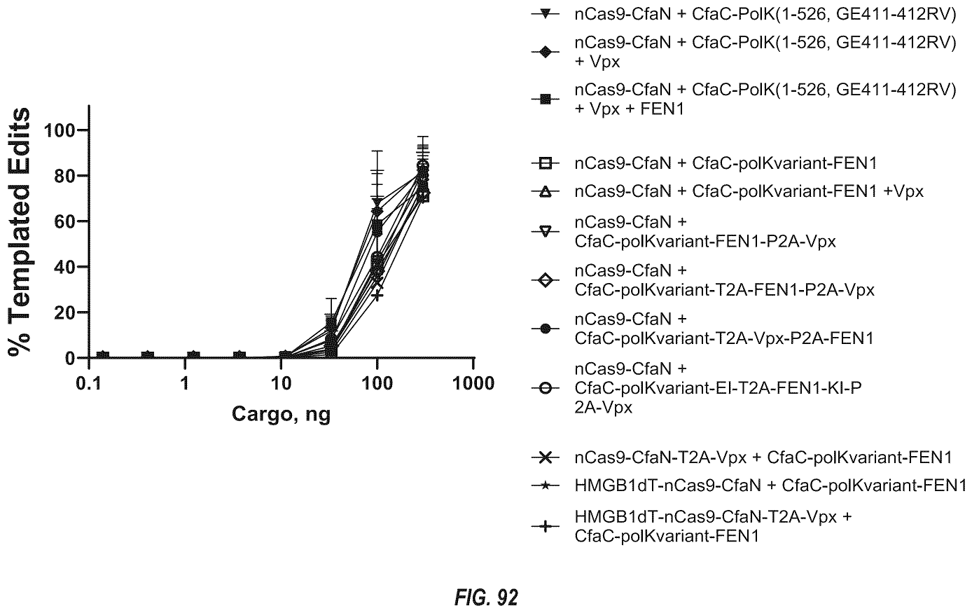

FIG. 92 and Table 95 show editing efficiencies obtained at the TTR locus in PCH treated with a tgRNA (G039267) and a ngRNA (G032630), along with split intein constructs comprising a nCas9 and a polymerase delivered in trans, with editing enhancers delivered in trans or in cis with the split intein constructs.

TABLE 95
Editing efficiencies obtained at the TTR locus in PCH treated with a tgRNA (G039267) and a ngRNA
(G032630), along with split intein constructs comprising a nCas9 and a polymerase delivered in
trans, with editing enhancers delivered in trans or in cis with the split intein constructs.

| | | | RNA cargo (ng) | | | | | | | |
|---|---|---|---|---|---|---|---|---|---|---|
| mRNA species | Edit Type | | 300 | 100 | 33.33 | 11.11 | 3.7 | 1.23 | 0.41 | 0.14 |
| nCas9-CfaN + | % Templated | Mean | 81.48 | 67.87 | 11.91 | 0.76 | 0.00 | 0.00 | 0.01* | 0.00 |
| CfaC- | Edits | SD | 7.17 | 13.07 | 6.46 | 0.38 | 0.00 | 0.00 | 0.01* | 0.00 |
| PolK(1-526, | % Templated + | Mean | 1.61 | 0.92 | 0.14 | 0.00 | 0.00 | 0.00 | 0.00* | 0.00 |
| GE411-412RV) | Byproduct Edits | SD | 0.82 | 0.28 | 0.11 | 0.00 | 0.00 | 0.00 | 0.00* | 0.00 |
| | % Indels | Mean | 0.97 | 0.50 | 0.05 | 0.00 | 0.00 | 0.00 | 0.00* | 0.01 |
| | | SD | 0.52 | 0.21 | 0.05 | 0.01 | 0.00 | 0.01 | 0.00* | 0.01 |
| nCas9-CfaN + | % Templated | Mean | 82.34 | 64.28 | 13.03 | 0.71 | 0.03 | 0.00 | 0.01* | 0.00 |
| CfaC- | Edits | SD | 7.95 | 18.03 | 6.19 | 0.34 | 0.02 | 0.01 | 0.01* | 0.00 |
| PolK(1-526, | % Templated + | Mean | 1.46 | 0.99 | 0.19 | 0.01 | 0.00 | 0.00 | 0.00* | 0.00 |
| GE411- | Byproduct Edits | SD | 0.61 | 0.38 | 0.13 | 0.01 | 0.01 | 0.00 | 0.00* | 0.00 |
| 412RV) + Vpx | % Indels | Mean | 1.45 | 0.60 | 0.10 | 0.00 | 0.01 | 0.00 | 0.01* | 0.00 |
| | | SD | 1.11 | 0.32 | 0.08 | 0.01 | 0.01 | 0.01 | 0.01* | 0.01 |
| nCas9-CfaN + | % Templated | Mean | 74.89 | 58.52 | 15.64 | 0.51 | 0.03 | 0.00 | 0.00* | 0.00 |
| CfaC- | Edits | SD | 22.20 | 32.24 | 10.44 | 0.44 | 0.03 | 0.00 | 0.00* | 0.00 |
| PolK(1-526, | % Templated + | Mean | 1.79 | 0.93 | 0.22 | 0.00 | 0.00 | 0.00 | 0.00* | 0.00 |
| GE411- | Byproduct Edits | SD | 0.19 | 0.59 | 0.18 | 0.01 | 0.00 | 0.00 | 0.00* | 0.00 |
| 412RV) + Vpx + | % Indels | Mean | 1.39 | 0.67 | 0.12 | 0.00 | 0.00 | 0.00 | 0.00* | 0.01 |
| FEN1 | | SD | 0.27 | 0.13 | 0.06 | 0.00 | 0.01 | 0.00 | 0.00* | 0.00 |
| nCas9-CfaN + | % Templated | Mean | 71.04 | 40.98 | 3.71 | 0.17 | 0.01 | 0.00 | 0.00* | 0.00 |
| CfaC- | Edits | SD | 16.18 | 24.53 | 2.99 | 0.09 | 0.02 | 0.00 | 0.00* | 0.00 |
| polKvariant- | % Templated + | Mean | 0.92 | 0.75 | 0.06 | 0.00 | 0.00 | 0.00 | 0.00* | 0.00 |
| FEN1 | Byproduct Edits | SD | 0.22 | 0.52 | 0.07 | 0.00 | 0.00 | 0.00 | 0.00* | 0.00 |
| | % Indels | Mean | 0.66 | 0.30 | 0.05 | 0.00 | 0.00 | 0.00 | 0.00* | 0.00 |
| | | SD | 0.21 | 0.41 | 0.03 | 0.00 | 0.00 | 0.00 | 0.00* | 0.01 |
| nCas9-CfaN + | % Templated | Mean | 82.17 | 38.94 | 3.46 | 0.10 | 0.01 | 0.00 | 0.00* | 0.00 |
| CfaC- | Edits | SD | 11.23 | 25.48 | 1.98 | 0.07 | 0.02 | 0.00 | 0.00* | 0.01 |
| polKvariant- | % Templated + | Mean | 1.47 | 0.57 | 0.05 | 0.00 | 0.00 | 0.00 | 0.00* | 0.00 |
| FEN1 + | Byproduct Edits | SD | 0.12 | 0.52 | 0.05 | 0.00 | 0.00 | 0.00 | 0.00* | 0.00 |
| Vpx | % Indels | Mean | 0.91 | 0.28 | 0.01 | 0.00 | 0.00 | 0.01 | 0.01* | 0.00 |
| | | SD | 0.20 | 0.25 | 0.01 | 0.00 | 0.00 | 0.01 | 0.01* | 0.01 |
| nCas9-CfaN + | % Templated | Mean | 78.04 | 42.44 | 3.52 | 0.09 | 0.00 | 0.00 | 0.00 | 0.01 |
| CfaC- | Edits | SD | 5.92 | 15.56 | 2.13 | 0.10 | 0.01 | 0.00 | 0.00 | 0.02 |
| polKvariant- | % Templated + | Mean | 1.32 | 0.60 | 0.04 | 0.00 | 0.00 | 0.00 | 0.00 | 0.00 |
| FEN1-P2A- | Byproduct Edits | SD | 0.40 | 0.11 | 0.03 | 0.00 | 0.00 | 0.00 | 0.00 | 0.00 |
| Vpx | % Indels | Mean | 0.85 | 0.41 | 0.03 | 0.00 | 0.00 | 0.00 | 0.01 | 0.00 |
| | | SD | 0.29 | 0.19 | 0.01 | 0.01 | 0.01 | 0.01 | 0.01 | 0.00 |
| nCas9-CfaN + | % Templated | Mean | 72.44 | 38.07 | 5.28 | 0.21 | 0.00 | 0.00 | 0.00* | 0.00 |
| CfaC- | Edits | SD | 17.74 | 32.82 | 6.56 | 0.10 | 0.00 | 0.00 | 0.00* | 0.01 |
| polKvariant- | % Templated + | Mean | 0.96 | 0.45 | 0.06 | 0.00 | 0.00 | 0.00 | 0.00* | 0.00 |
| T2A-FEN1- | Byproduct Edits | SD | 0.38 | 0.64 | 0.09 | 0.00 | 0.00 | 0.00 | 0.00* | 0.00 |
| P2A-Vpx | % Indels | Mean | 0.80 | 0.32 | 0.03 | 0.01 | 0.00 | 0.00 | 0.01* | 0.00 |
| | | SD | 0.42 | 0.54 | 0.04 | 0.01 | 0.01 | 0.01 | 0.01* | 0.01 |
| nCas9-CfaN + | % Templated | Mean | 81.03 | 55.15 | 7.66 | 0.33 | 0.01 | 0.00 | 0.00* | 0.00 |
| CfaC- | Edits | SD | 4.12 | 21.01 | 6.46 | 0.33 | 0.00 | 0.00 | 0.00* | 0.00 |
| polKvariant- | % Templated + | Mean | 1.19 | 0.71 | 0.10 | 0.00 | 0.00 | 0.00 | 0.00* | 0.00 |
| T2A-Vpx- | Byproduct Edits | SD | 0.04 | 0.35 | 0.11 | 0.00 | 0.00 | 0.00 | 0.00* | 0.00 |
| P2A-FEN1 | % Indels | Mean | 0.75 | 0.53 | 0.03 | 0.01 | 0.01 | 0.00 | 0.00* | 0.00 |
| | | SD | 0.03 | 0.48 | 0.04 | 0.01 | 0.01 | 0.01 | 0.00* | 0.01 |
| nCas9-CfaN + | % Templated | Mean | 84.41 | 44.24 | 8.33 | 0.30 | 0.03 | 0.00 | 0.00* | 0.00 |
| CfaC- | Edits | SD | 7.39 | 25.47 | 8.23 | 0.36 | 0.04 | 0.00 | 0.00* | 0.00 |
| polKvariant- | % Templated + | Mean | 1.30 | 0.87 | 0.10 | 0.00 | 0.00 | 0.00 | 0.00* | 0.00 |
| EI-T2A-FEN1- | Byproduct Edits | SD | 0.28 | 0.37 | 0.13 | 0.00 | 0.00 | 0.00 | 0.00* | 0.00 |
| KI-P2A-Vpx | % Indels | Mean | 0.90 | 0.53 | 0.05 | 0.00 | 0.01 | 0.01 | 0.01* | 0.00 |
| | | SD | 0.35 | 0.10 | 0.04 | 0.00 | 0.01 | 0.01 | 0.01* | 0.01 |
| nCas9-CfaN-T2A- | % Templated | Mean | 76.17 | 32.99 | 2.61 | 0.06 | 0.00 | 0.00 | 0.01 | 0.03 |
| Vpx + CfaC- | Edits | SD | 6.81 | 21.36 | 3.24 | 0.06 | 0.00 | 0.00 | 0.01 | 0.06 |
| polKvariant- | % Templated + | Mean | 1.32 | 0.56 | 0.04 | 0.00 | 0.00 | 0.00 | 0.00 | 0.00 |
| FEN1 | Byproduct Edits | SD | 0.76 | 0.42 | 0.05 | 0.00 | 0.00 | 0.00 | 0.00 | 0.00 |
| | % Indels | Mean | 0.56 | 0.28 | 0.02 | 0.01 | 0.00 | 0.01 | 0.00 | 0.00 |
| | | SD | 0.05 | 0.23 | 0.03 | 0.01 | 0.00 | 0.01 | 0.01 | 0.00 |
| HMGB1dT-nCas9- | % Templated | Mean | 75.64 | 35.79* | 3.88* | 0.64 | 0.03 | 0.01 | 0.00 | 0.00* |
| CfaN + CfaC- | Edits | SD | 16.90 | 4.54* | N/A | 0.58 | 0.04 | 0.01 | 0.00 | 0.00* |
| polKvariant- | % Templated + | Mean | 0.97 | 0.88 | 0.19* | 0.00 | 0.00 | 0.00 | 0.00 | 0.00* |
| FEN1 | Byproduct Edits | SD | 0.23 | 1.02 | 0.22* | 0.01 | 0.00 | 0.00 | 0.00 | 0.00* |
| | % Indels | Mean | 0.71 | 0.30 | 0.08* | 0.00 | 0.01 | 0.00 | 0.01 | 0.02* |
| | | SD | 0.30 | 0.47 | 0.05* | 0.01 | 0.01 | 0.01 | 0.01 | 0.01* |
| HMGB1dT-nCas9- | % Templated | Mean | 70.39 | 27.50 | 1.53 | 0.06 | 0.00 | 0.04 | 0.00 | 0.00 |
| CfaN-T2A-Vpx + | Edits | SD | 13.24 | 22.39 | 1.75 | 0.05 | 0.01 | 0.06 | 0.00 | 0.00 |
| CfaC- | % Templated + | Mean | 0.90 | 0.33 | 0.10 | 0.00 | 0.00 | 0.00 | 0.00 | 0.00 |
| polKvariant- | Byproduct Edits | SD | 0.21 | 0.29 | 0.18 | 0.00 | 0.00 | 0.00 | 0.00 | 0.00 |
| FEN1 | % Indels | Mean | 0.71 | 0.18 | 0.01 | 0.01 | 0.00 | 0.01 | 0.01 | 0.00 |
| | | SD | 0.28 | 0.20 | 0.01 | 0.01 | 0.00 | 0.01 | 0.00 | 0.01 |

*N = 2

### Example 46—Editing at the VEGFA Locus in PHH Using Constructs Comprising a SpyCas9 Nickase, a Polymerase Kappa Variant, HMGB1, and Editing Enhancers Operably Linked to Split Inteins

This Example evaluates the editing efficiency of a SpyCas9 H840A nickase (nCas9) fused with an HMGB1 moiety. The fusion construct was delivered in trans with a polymerase in the presence of editing enhancers, wherein the editing enhancers are delivered (i) in trans, or (ii) in cis with either the polymerase or the nCas9.

Primary human hepatocytes (PHH, Gibco, Lot 8381) were prepared as described in Example 1, and plated on a collagen I coated 96-well plate (Stem cell technologies, Cat. 100-0366) at a density of 33,000 cells/well. LNPs were prepared as described in Example 1 to deliver RNA cargo to the cells. The LNPs were made with a molar ratio of 50% Lipid A, 38% cholesterol, 9% DSPC, and 3% PEG2k-DMG, and formulated with a lipid amine to RNA phosphate (N:P) molar ratio of about 6.

The cells were treated with a total RNA cargo comprising a tgRNA cargo and a mRNA cargo. The tgRNA cargo, delivered via a LNP, comprised a tgRNA (G032596). The tgRNA comprised a spacer sequence targeting the VEGFA locus, a scaffold with the structure shown FIG. 6E, and a 3′ extension comprising a template encoding a GAGG-to-CACT edit, and a DRS. The mRNA cargo consisted of one of the following cargos:
a LNP comprising a mRNA encoding a nCas9-CfaN fusion protein (SEQ ID NO: 1362) comprising a nCas9 moiety and a CfaN split intein, and a mRNA encoding a CfaC-PolK(1-526, GE411-412RV) fusion protein (SEQ ID NO: 1368) comprising a CfaC split intein capable of undergoing a trans-splicing reaction with a CfaN split intein and a PolK variant moiety, wherein the nCas9:polymerase mass ratio was 5:1,
a LNP comprising a mRNA encoding a nCas9-CfaN fusion protein (SEQ ID NO: 1362), and a mRNA encoding a CfaC-PolK(1-526, GE411-412RV) fusion protein (SEQ ID NO: 1368), wherein the nCas9:polymerase mass ratio was 5:1, a LNP comprising a mRNA encoding Vpx (SEQ ID NO: 1462), and a LNP comprising a mRNA encoding FEN1 (SEQ ID NO: 1465), wherein the LNPs were delivered in a nCas9:polymerase:Vpx:FEN mass ratio of 10:2:1:1,
a LNP comprising a mRNA encoding a nCas9-CfaN-T2A-Vpx fusion protein (SEQ ID NO: 1277) comprising a nCas9 moiety, a CfaN split intein, a T2A peptide, and a Vpx moiety, and a LNP comprising a mRNA encoding a CfaC-PolK(1-526, GE411-412RV) fusion protein (SEQ ID NO: 1368), wherein the LNPs were delivered in a nCas9:polymerase mass ratio of 5:1,
a LNP comprising a mRNA encoding a nCas9-CfaN-T2A-Vpx fusion protein (SEQ ID NO: 1277), a LNP comprising a mRNA encoding a CfaC-PolK(1-526, GE411-412RV) fusion protein (SEQ ID NO: 1368), and a LNP comprising a mRNA encoding FEN1 (SEQ ID NO: 1465), wherein the LNPs were delivered in a nCas9:polymerase:FEN1 mass ratio of 5:1:1,
a LNP comprising a mRNA encoding a nCas9-CfaN-T2A-Vpx fusion protein (SEQ ID NO: 1277), and a LNP comprising a mRNA encoding a CfaC-polKvariant-FEN1 fusion protein (SEQ ID NO: 1283), comprising a CfaC split intein, a polymerase Kappa variant moiety, and a FEN1 moiety, wherein the LNPs were delivered in a nCas9:polymerase mass ratio of 5:1,
a LNP comprising a mRNA encoding a HMGBldT-nCas9-CfaN fusion protein (SEQ ID NO: 1365) comprising HMGB1_dTail, a nCas9 moiety and a CfaN split intein, and a LNP comprising a mRNA encoding a CfaC-PolK(1-526, GE411-412RV) fusion protein (SEQ ID NO: 1368), wherein the LNPs were delivered in a nCas9:polymerase mass ratio of 5:1,
a LNP comprising a mRNA encoding a HMGB1dT-nCas9-CfaN fusion protein (SEQ ID NO: 1365), a LNP comprising a mRNA encoding a CfaC-PolK(1-526, GE411-412RV) fusion protein (SEQ ID NO: 1368), and a LNP comprising a mRNA encoding FEN1 (SEQ ID NO: 1465), wherein the LNPs were delivered in a nCas9:polymerase:FEN1 mass ratio of 5:1:1,
a LNP comprising a mRNA encoding a HMGBldT-nCas9-CfaN fusion protein (SEQ ID NO: 1365), a LNP comprising a mRNA encoding a CfaC-PolK(1-526, GE411-412RV) fusion protein (SEQ ID NO: 1368), a LNP comprising a mRNA encoding Vpx (SEQ ID NO: 1462), and a LNP comprising a mRNA encoding FEN1 (SEQ ID NO: 1465), wherein the LNPs were delivered in a nCas9:polymerase:Vpx:FEN mass ratio of 10:2:1:1,
a LNP comprising a mRNA encoding a HMGBldT-nCas9-CfaN fusion protein (SEQ ID NO: 1365), and a LNP comprising a mRNA encoding a CfaC-polKvariant-FEN1 fusion protein (SEQ ID NO: 1283), wherein the LNPs were delivered in a nCas9:polymerase mass ratio of 5:1,
a LNP comprising a mRNA encoding a HMGBldT-nCas9-CfaN-T2A-Vpx fusion protein (SEQ ID NO: 1280) comprising HMGB1_dTail, a nCas9 moiety, a CfaN split intein, a T2A peptide, and a Vpx moiety, and a LNP comprising a mRNA encoding a CfaC-PolK(1-526, GE411-412RV) fusion protein (SEQ ID NO: 1368), wherein the LNPs were delivered in a nCas9:polymerase mass ratio of 5:1,
a LNP comprising a mRNA encoding a HMGBldT-nCas9-CfaN-T2A-Vpx fusion protein (SEQ ID NO: 1280), a LNP comprising a mRNA encoding a CfaC-PolK(1-526, GE411-412RV) fusion protein (SEQ ID NO: 1368), and a LNP comprising a mRNA encoding FEN1 (SEQ ID NO: 1465), wherein the LNPs were delivered in a nCas9:polymerase:FEN1 mass ratio of 5:1:1, or
a LNP comprising a mRNA encoding a HMGBldT-nCas9-CfaN-T2A-Vpx fusion protein (SEQ ID NO: 1280), and a LNP comprising a mRNA encoding a CfaC-polKvariant-FEN1 fusion protein (SEQ ID NO: 1283), wherein the LNPs were delivered in a nCas9:polymerase mass ratio of 5:1.

Cells were incubated at 37° C., 5% CO₂ for 24 hours prior to treatment with LNPs. LNPs were incubated in media containing 10% fetal bovine serum (FBS) at 37° C. for 10 minutes. Post-incubation, the LNPs were delivered to the cells, in an 8-point 3-fold dilution series starting at 300 ng total RNA cargo, wherein the mass ratio of tgRNA to mRNA encoding nCas9 and polymerase was kept constant at 1:2. The cells were lysed 72 hours post-treatment for NGS analysis as described in Example 1. Samples were run in duplicates, unless otherwise indicated.

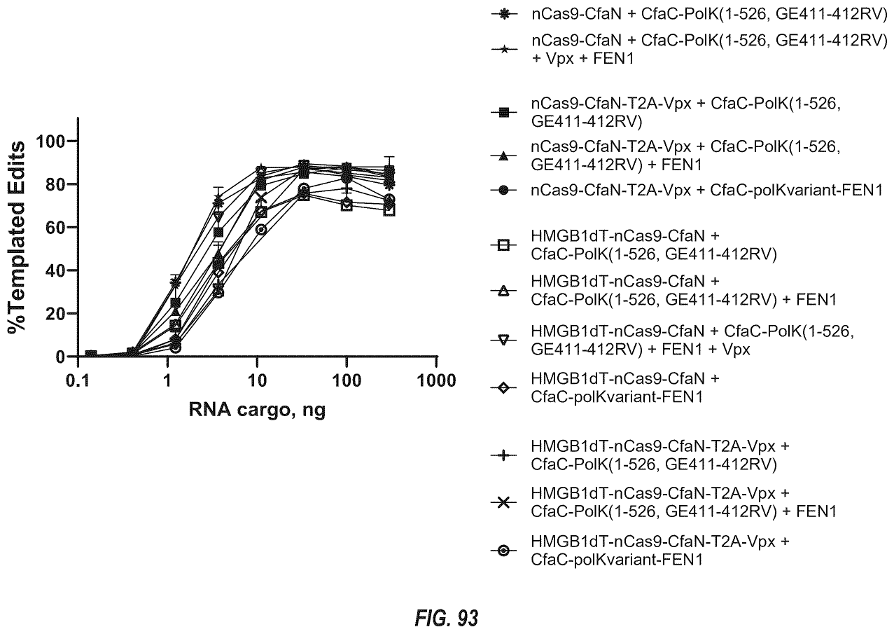

FIG. 93 and Table 96 show editing efficiencies obtained at the VEGFA locus in PHH treated with a tgRNA (G032596), along with split intein constructs comprising a nCas9 fused with HMGB1_dTail, and a polymerase delivered in trans, with editing enhancers delivered in trans or in cis with the split intein constructs.

TABLE 96
Editing efficiencies obtained at the VEGFA locus in PHH treated with a tgRNA (G032596), along with
split intein constructs comprising a nCas9 fused with HMGB1_dTail, and a polymerase delivered
in trans, with editing enhancers delivered in trans or in cis with the split intein constructs.

| | | | RNA cargo (ng) | | | | | | | |
|---|---|---|---|---|---|---|---|---|---|---|
| mRNA species | Edit Type | | 300 | 100 | 33.33 | 11.11 | 3.7 | 1.23 | 0.41 | 0.14 |
| nCas9-CfaN + | % Templated | Mean | 79.53 | 83.59* | 85.58 | 82.82 | 71.12* | 34.33* | 2.05 | 0.37 |
| CfaC- | Edits | SD | 1.34 | N/A | 1.99 | 2.50 | N/A | N/A | 1.75 | 0.16 |
| PolK(1-526, | % Templated + | Mean | 1.97 | 1.91* | 1.85 | 2.04 | 1.44* | 0.92* | 0.03 | 0.00 |
| GE411-412RV) | Byproduct Edits | SD | 0.15 | N/A | 0.18 | 0.45 | N/A | N/A | 0.03 | 0.00 |
| | % Indels | Mean | 0.81 | 1.05* | 0.58 | 0.45 | 0.21* | 0.19* | 0.01 | 0.05 |
| | | SD | 0.08 | N/A | 0.18 | 0.11 | N/A | N/A | 0.01 | 0.04 |
| nCas9-CfaN + | % Templated | Mean | 87.93 | 87.91 | 87.79 | 87.64 | 74.14 | 32.92 | 0.17* | 0.13 |
| CfaC- | Edits | SD | 4.82 | 1.99 | 1.38 | 0.37 | 4.37 | 4.99 | N/A | 0.16 |
| PolK(1-526, | % Templated + | Mean | 2.23 | 3.10 | 2.47 | 2.79 | 1.67 | 0.64 | 0.00* | 0.00 |
| GE411-412RV) + | Byproduct Edits | SD | 0.32 | 0.33 | 0.16 | 0.18 | 0.15 | 0.01 | N/A | 0.00 |
| Vpx + FEN1 | % Indels | Mean | 0.45 | 0.69 | 0.65 | 0.82 | 0.73 | 0.41 | 0.00* | 0.03 |
| | | SD | 0.30 | 0.04 | 0.36 | 0.25 | 0.49 | 0.46 | N/A | 0.03 |
| nCas9-CfaN-T2A- | % Templated | Mean | 86.25* | 87.53 | 84.84* | 79.33 | 57.70 | 25.07 | 1.99 | 0.21 |
| Vpx + CfaC- | Edits | SD | N/A | 0.47 | N/A | 2.47 | 8.13 | 10.65 | 2.19 | 0.25 |
| PolK(1-526, | % Templated + | Mean | 2.95* | 1.86 | 2.00* | 1.93 | 1.40 | 0.32 | 0.03 | 0.00 |
| GE411-412RV) | Byproduct Edits | SD | N/A | 0.17 | N/A | 0.65 | 0.23 | 0.45 | 0.03 | 0.00 |
| | % Indels | Mean | 0.18* | 0.62 | 0.34* | 0.45 | 0.30 | 0.00 | 0.01 | 0.01 |
| | | SD | N/A | 0.28 | N/A | 0.33 | 0.32 | 0.00 | 0.01 | 0.01 |
| nCas9-CfaN-T2A- | % Templated | Mean | 84.07 | 88.36* | 89.41* | 83.62* | 47.89* | 21.11* | 1.51 | 0.50* |
| Vpx + CfaC- | Edits | SD | 4.02 | N/A | N/A | N/A | N/A | N/A | 1.51 | N/A |
| PolK(1-526, | % Templated + | Mean | 2.25 | 2.40* | 2.47* | 1.83* | 1.21* | 0.37* | 0.05 | 0.01* |
| GE411-412RV) + | Byproduct Edits | SD | 0.23 | N/A | N/A | N/A | N/A | N/A | 0.05 | N/A |
| FEN1 | % Indels | Mean | 0.42 | 0.51* | 0.47* | 0.49* | 0.20* | 0.05* | 0.01 | 0.01* |
| | | SD | 0.05 | N/A | N/A | N/A | N/A | N/A | 0.01 | N/A |
| nCas9-CfaN-T2A- | % Templated | Mean | 83.26 | 87.89* | 86.92 | N/A | 42.82 | 8.01* | 1.00* | 0.02* |
| Vpx + CfaC- | Edits | SD | 2.39 | N/A | 0.39 | N/A | 10.39 | N/A | N/A | N/A |
| polKvariant-FEN1 | % Templated + | Mean | 2.10 | 2.45* | 2.32 | N/A | 0.81 | 0.18* | 0.01* | 0.00* |
| | Byproduct Edits | SD | 0.02 | N/A | 0.45 | N/A | 0.20 | N/A | N/A | N/A |
| | % Indels | Mean | 0.83 | 0.73* | 0.70 | N/A | 0.28 | 0.02* | 0.01* | 0.00* |
| | | SD | 0.00 | N/A | 0.06 | N/A | 0.07 | N/A | N/A | N/A |
| HMGB1dT-nCas9- | % Templated | Mean | 67.81 | 70.13 | 74.82 | 67.09* | 43.43* | 14.31* | 1.31 | 0.11* |
| CfaN + CfaC- | Edits | SD | 3.70 | 1.34 | 1.55 | N/A | N/A | N/A | 1.05 | N/A |
| PolK(1-526, | % Templated + | Mean | 1.64 | 1.73 | 1.97 | 1.44* | 1.00* | 0.45* | 0.02 | 0.00* |
| GE411-412RV) | Byproduct Edits | SD | 0.08 | 0.14 | 0.25 | N/A | N/A | N/A | 0.01 | N/A |
| | % Indels | Mean | 1.52 | 1.84 | 1.38 | 0.78* | 0.51* | 0.17* | 0.02 | 0.00* |
| | | SD | 0.04 | 0.50 | 0.40 | N/A | N/A | N/A | 0.01 | N/A |
| HMGB1dT-nCas9- | % Templated | Mean | 81.68 | 84.22* | 87.87* | 81.78 | N/A | 15.05* | 1.90* | 0.16* |
| CfaN + CfaC- | Edits | SD | 1.27 | N/A | N/A | 2.84 | N/A | N/A | N/A | N/A |
| PolK(1-526, | % Templated + | Mean | 2.13 | 2.61* | 2.47* | 1.81 | N/A | 0.36* | 0.04* | 0.00* |
| GE411-412RV) + | Byproduct Edits | SD | 0.01 | N/A | N/A | 0.10 | N/A | N/A | N/A | N/A |
| FEN1 | % Indels | Mean | 1.12 | 1.91* | 1.34* | 0.75 | N/A | 0.07* | 0.01* | 0.01* |
| | | SD | 0.01 | N/A | N/A | 0.01 | N/A | N/A | N/A | N/A |
| HMGB1dT-nCas9- | % Templated | Mean | 84.79* | N/A | 88.55* | 85.22 | 64.68 | N/A | 1.52 | 0.12 |
| CfaN + CfaC- | Edits | SD | N/A | N/A | N/A | 2.00 | 6.04 | N/A | 1.51 | 0.06 |
| PolK(1-526, | % Templated + | Mean | 2.69* | N/A | 2.66* | 2.45 | 1.66 | N/A | 0.03 | 0.00 |
| GE411-412RV) + | Byproduct Edits | SD | N/A | N/A | N/A | 0.01 | 0.21 | N/A | 0.02 | 0.00 |
| FEN1 + Vpx | % Indels | Mean | 0.85* | N/A | 0.93* | 0.87 | 0.56 | N/A | 0.01 | 0.00 |
| | | SD | N/A | N/A | N/A | 0.01 | 0.36 | N/A | 0.01 | 0.00 |
| HMGB1dT-nCas9- | % Templated | Mean | 70.59 | 71.52 | 75.65 | 67.54 | 39.04 | 8.07 | 0.33 | 0.03 |
| CfaN + CfaC- | Edits | SD | 0.58 | 4.35 | 1.46 | 4.89 | 12.62 | 3.46 | 0.29 | 0.04 |
| polKvariant-FEN1 | | Mean | 1.79 | 1.64 | 1.91 | 1.76 | 0.91 | 0.13 | 0.01 | 0.00 |
| | % Templated + | SD | 0.11 | 0.40 | 0.07 | 0.14 | 0.19 | 0.07 | 0.01 | 0.00 |
| | Byproduct Edits | | | | | | | | | |
| | % Indels | Mean | 1.34 | 1.54 | 1.31 | 1.16 | 0.39 | 0.06 | 0.01 | 0.01 |
| | | SD | 0.05 | 0.13 | 0.08 | 0.25 | 0.03 | 0.06 | 0.01 | 0.00 |
| HMGB1dT-nCas9- | % Templated | Mean | 72.35 | 77.99 | 75.79 | N/A | 33.50 | 5.85* | 0.71 | 0.01 |
| CfaN-T2A- | Edits | SD | 0.21 | 1.06 | 1.02 | N/A | 10.61 | N/A | 0.59 | 0.01 |
| Vpx + CfaC- | % Templated + | Mean | 1.70 | 2.00 | 1.82 | N/A | 0.75 | 0.13* | 0.01 | 0.00 |
| PolK(1-526, | Byproduct Edits | SD | 0.25 | 0.12 | 0.13 | N/A | 0.28 | N/A | 0.01 | 0.00 |
| GE411-412RV) | % Indels | Mean | 1.15 | 1.32 | 0.99 | N/A | 0.35 | 0.02* | 0.07 | 0.01 |
| | | SD | 0.34 | 0.22 | 0.08 | N/A | 0.11 | N/A | 0.08 | 0.01 |
| HMGB1dT-nCas9- | % Templated | Mean | 83.11 | N/A | 87.11* | 73.79 | 30.50* | 6.28* | 0.93 | 0.00 |
| CfaN-T2A- | Edits | SD | 0.49 | N/A | N/A | 7.04 | N/A | N/A | 0.71 | 0.00 |
| Vpx + CfaC- | % Templated + | Mean | 2.28 | N/A | 2.12* | 1.67 | 0.68* | 0.12* | 0.02 | 0.00 |
| PolK(1-526, | Byproduct Edits | SD | 0.04 | N/A | N/A | 0.46 | N/A | N/A | 0.01 | 0.00 |
| GE411-412RV) + | % Indels | Mean | 0.94 | N/A | 0.69* | 0.72 | 0.19* | 0.04* | 0.02 | 0.00 |
| FEN1 | | SD | 0.10 | N/A | N/A | 0.11 | N/A | N/A | 0.01 | 0.00 |
| HMGB1dT-nCas9- | % Templated | Mean | 72.94* | 82.85* | 77.94* | 58.89* | 29.64 | 4.11 | 0.16* | 0.00 |
| CfaN-T2A- | Edits | SD | N/A | N/A | N/A | N/A | 8.91 | 0.30 | N/A | 0.00 |
| Vpx + CfaC- | % Templated + | Mean | 1.96* | 2.08* | 2.37* | 1.30* | 0.63 | 0.16 | 0.00* | 0.00 |
| polKvariant-FEN1 | Byproduct Edits | SD | N/A | N/A | N/A | N/A | 0.22 | 0.11 | N/A | 0.00 |
| | % Indels | Mean | 1.33* | 1.19* | 0.94* | 0.73* | 0.44 | 0.10 | 0.00* | 0.01 |
| | | SD | N/A | N/A | N/A | N/A | 0.02 | 0.05 | N/A | 0.00 |

*N = 1

### Example 47—Editing at the TTR Locus in PCH Using Constructs Comprising a SpyCas9 Nickase, a Polymerase Kappa Variant, HMGB1, and Editing Enhancers Operably Linked to Split Inteins

This Example evaluated the editing efficiency of a SpyCas9 H840A nickase (nCas9) fused with HMGB1_dTail, and delivered in trans with a polymerase in the presence of editing enhancers, wherein the editing enhancers are delivered (i) in trans, or (ii) in cis with either the polymerase or the nCas9.

Primary cynomolgus hepatocytes (PCH, Gibco, Lot CY423) were prepared as described in Example 1, and plated on a collagen I coated 96-well plate (Stem cell technologies, Cat. 100-0366) at a density of 40,000 cells/well. LNPs were prepared as described in Example 1 to deliver RNA cargo to the cells. The LNPs were made with a molar ratio of 50% Lipid A, 38% cholesterol, 9% DSPC, and 3% PEG2k-DMG, and formulated with a lipid amine to RNA phosphate (N:P) molar ratio of about 6.

The cells were treated with a total RNA cargo comprising a tgRNA cargo, a ngRNA cargo and a mRNA cargo. The tgRNA cargo, delivered via a LNP, comprised a tgRNA (G039267). The tgRNA comprised a spacer sequence targeting the TTR locus, a scaffold with the structure shown FIG. 6C, and a 3′ extension comprising a template encoding a TG-to-AC edit, and a DRS. The ngRNA cargo, delivered via a LNP, comprised a ngRNA (G032630). The mRNA cargo consisted of one of the following cargos:
a LNP comprising a mRNA encoding a nCas9-CfaN fusion protein (SEQ ID NO: 1362) comprising a nCas9 moiety and a CfaN split intein, and a mRNA encoding a CfaC-PolK(1-526, GE411-412RV) fusion protein (SEQ ID NO: 1368) comprising a CfaC split intein capable of undergoing a trans-splicing reaction with a CfaN split intein and a PolK variant moiety, wherein the nCas9:polymerase mass ratio was 5:1,
a LNP comprising a mRNA encoding a nCas9-CfaN fusion protein (SEQ ID NO: 1362), and a mRNA encoding a CfaC-PolK(1-526, GE411-412RV) fusion protein (SEQ ID NO: 1368), wherein the nCas9:polymerase mass ratio was 5:1, a LNP comprising a mRNA encoding Vpx (SEQ ID NO: 1462), and a LNP comprising a mRNA encoding FEN1 (SEQ ID NO: 1465), wherein the LNPs were delivered in a nCas9:polymerase:Vpx:FEN mass ratio of 10:2:1:1,
a LNP comprising a mRNA encoding a nCas9-CfaN-T2A-Vpx fusion protein (SEQ ID NO: 1277) comprising a nCas9 moiety, a CfaN split intein, a T2A peptide, and a Vpx moiety, and a LNP comprising a mRNA encoding a CfaC-PolK(1-526, GE411-412RV) fusion protein (SEQ ID NO: 1368), wherein the LNPs were delivered in a nCas9:polymerase mass ratio of 5:1,
a LNP comprising a mRNA encoding a nCas9-CfaN-T2A-Vpx fusion protein (SEQ ID NO: 1277), a LNP comprising a mRNA encoding a CfaC-PolK(1-526, GE411-412RV) fusion protein (SEQ ID NO: 1368), and a LNP comprising a mRNA encoding FEN1 (SEQ ID NO: 1465), wherein the LNPs were delivered in a nCas9:polymerase:FEN1 mass ratio of 5:1:1,
a LNP comprising a mRNA encoding a nCas9-CfaN-T2A-Vpx fusion protein (SEQ ID NO: 1277), and a LNP comprising a mRNA encoding a CfaC-polKvariant-FEN1 fusion protein (SEQ ID NO: 1283), comprising a CfaC split intein, a polymerase Kappa variant moiety, and a FEN1 moiety, wherein the LNPs were delivered in a nCas9:polymerase mass ratio of 5:1,
a LNP comprising a mRNA encoding a HMGB1dT-nCas9-CfaN fusion protein (SEQ ID NO: 1365) comprising HMGB1_dTail, a nCas9 moiety and a CfaN split intein, and a LNP comprising a mRNA encoding a CfaC-PolK(1-526, GE411-412RV) fusion protein (SEQ ID NO: 1368), wherein the LNPs were delivered in a nCas9:polymerase mass ratio of 5:1,
a LNP comprising a mRNA encoding a HMGB1dT-nCas9-CfaN fusion protein (SEQ ID NO: 1365), a LNP comprising a mRNA encoding a CfaC-PolK(1-526, GE411-412RV) fusion protein (SEQ ID NO: 1368), and a LNP comprising a mRNA encoding FEN1 (SEQ ID NO: 1465), wherein the LNPs were delivered in a nCas9:polymerase:FEN1 mass ratio of 5:1:1,
a LNP comprising a mRNA encoding a HMGB1dT-nCas9-CfaN fusion protein (SEQ ID NO: 1365), a LNP comprising a mRNA encoding a CfaC-PolK(1-526, GE411-412RV) fusion protein (SEQ ID NO: 1368), a LNP comprising a mRNA encoding Vpx (SEQ ID NO: 1462), and a LNP comprising a mRNA encoding FEN1 (SEQ ID NO: 1465), wherein the LNPs were delivered in a nCas9:polymerase:Vpx:FEN mass ratio of 10:2:1:1,
a LNP comprising a mRNA encoding a HMGBldT-nCas9-CfaN fusion protein (SEQ ID NO: 1365), and a LNP comprising a mRNA encoding a CfaC-polKvariant-FEN1 fusion protein (SEQ ID NO: 1283), wherein the LNPs were delivered in a nCas9:polymerase mass ratio of 5:1,
a LNP comprising a mRNA encoding a HMGBldT-nCas9-CfaN-T2A-Vpx fusion protein (SEQ ID NO: 1280) comprising HMGB1_dTail, a nCas9 moiety, a CfaN split intein, a T2A peptide, and a Vpx moiety, and a LNP comprising a mRNA encoding a CfaC-PolK(1-526, GE411-412RV) fusion protein (SEQ ID NO: 1368), wherein the LNPs were delivered in a nCas9:polymerase mass ratio of 5:1,
a LNP comprising a mRNA encoding a HMGBldT-nCas9-CfaN-T2A-Vpx fusion protein (SEQ ID NO: 1280), a LNP comprising a mRNA encoding a CfaC-PolK(1-526, GE411-412RV) fusion protein (SEQ ID NO: 1368), and a LNP comprising a mRNA encoding FEN1 (SEQ ID NO: 1465), wherein the LNPs were delivered in a nCas9:polymerase:FEN1 mass ratio of 5:1:1, or
a LNP comprising a mRNA encoding a HMGBldT-nCas9-CfaN-T2A-Vpx fusion protein (SEQ ID NO: 1280), and a LNP comprising a mRNA encoding a CfaC-polKvariant-FEN1 fusion protein (SEQ ID NO: 1283), wherein the LNPs were delivered in a nCas9:polymerase mass ratio of 5:1.

Cells were incubated at 37° C., 5% CO₂ for 24 hours prior to treatment with LNPs. LNPs were incubated in media containing 10% fetal bovine serum (FBS) at 37° C. for 10 minutes. Post-incubation, the LNPs were delivered to the cells, in a 7-point 3-fold dilution series starting at 300 ng total RNA cargo, wherein the mass ratio of tgRNA to ngRNA was kept constant at 4:1 and the mass ratio of gRNA (i.e., tgRNA and ngRNA) to mRNA encoding nCas9 and polymerase was kept constant at 1:2. The cells were lysed 72 hours post-treatment for NGS analysis as described in Example 1. Samples were run in duplicates, unless otherwise indicated.

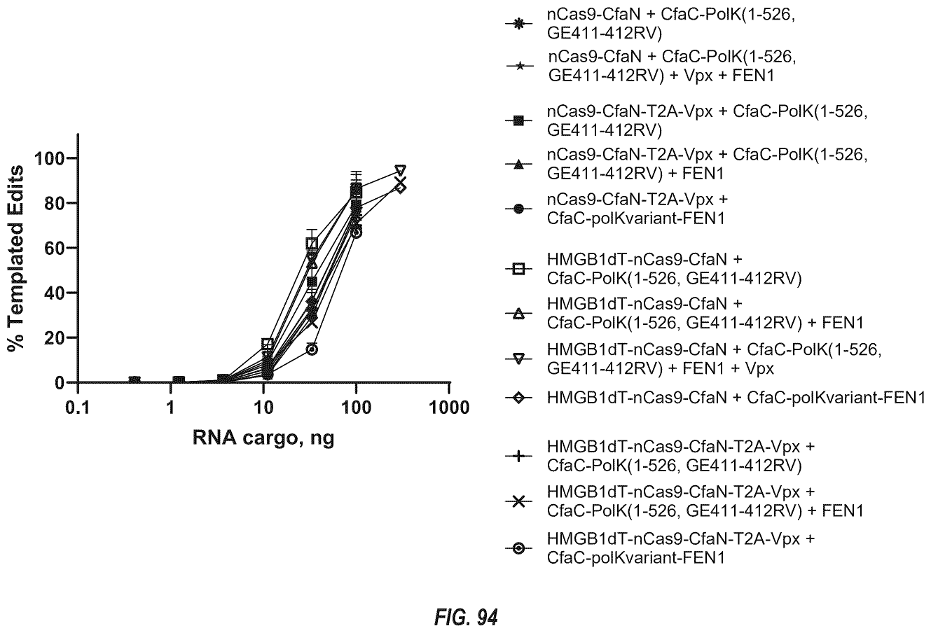

FIG. 94 and Table 97 show editing efficiencies obtained at the TTR locus in PCH treated with a tgRNA (G039267) and a ngRNA (G032630), along with split intein constructs comprising a nCas9 fused with HMGB1_dTail, and a polymerase delivered in trans, with editing enhancers delivered in trans or in cis with the split intein constructs.

TABLE 97
Editing efficiencies obtained at the TTR locus in PCH treated with a (G039267) and a ngRNA (G032630),
along with split intein constructs comprising a nCas9 fused with HMGB1_dTail, and a polymerase delivered
in trans, with editing enhancers delivered in trans or in cis with the split intein constructs.

| | | RNA | | | | | | | |
|---|---|---|---|---|---|---|---|---|---|
| mRNA species | Edit Type | cargo (ng) | 300 | 100 | 33.33 | 11.11 | 3.7 | 1.23 | 0.41 |
| nCas9-CfaN + CfaC- | % Templated | Mean | N/A | 74.62 | 32.01 | 5.08 | 0.0 | 0.01* | N/A |
| PolK(1-526, GE411- | Edits | SD | N/A | 7.83 | 0.16 | 0.76 | 0.01 | N/A | N/A |
| 412RV) | % Templated + | Mean | N/A | 1.40 | 0.28 | 0.04 | 0.00 | 0.00* | N/A |
| | Byproduct Edits | SD | N/A | 0.02 | 0.23 | 0.01 | 0.00 | N/A | N/A |
| | % Indels | Mean | N/A | 1.59 | 0.36 | 0.01 | 0.00 | 0.01* | N/A |
| | | SD | N/A | 1.10 | 0.44 | 0.00 | 0.00 | N/A | N/A |
| nCas9-CfaN + CfaC- | % Templated | Mean | N/A | 77.61 | 36.59 | 6.34 | 0.45 | 0.00* | N/A |
| PolK(1-526, GE411- | Edits | SD | N/A | 15.06 | 9.55 | 1.68 | 0.16 | N/A | N/A |
| 412RV) + Vpx + FEN1 | % Templated + | Mean | N/A | 1.36 | 0.77 | 0.11 | 0.00 | 0.00* | N/A |
| | Byproduct Edits | SD | N/A | 0.49 | 0.56 | 0.06 | 0.00 | N/A | N/A |
| | % Indels | Mean | N/A | 1.09 | 0.14 | 0.01 | 0.00 | 0.00* | N/A |
| | | SD | N/A | 0.70 | 0.20 | 0.01 | 0.00 | N/A | N/A |
| nCas9-CfaN-T2A- | % Templated | Mean | N/A | 79.06 | 44.97 | 8.81 | 0.73 | 0.08 | N/A |
| Vpx + CfaC-PolK(1- | Edits | SD | N/A | 5.56 | 12.81 | 4.57 | 0.18 | 0.02 | N/A |
| 526, GE411-412RV) | % Templated + | Mean | N/A | 2.31 | 0.71 | 0.09 | 0.01 | 0.00 | N/A |
| | Byproduct Edits | SD | N/A | 1.07 | 0.05 | 0.06 | 0.01 | 0.00 | N/A |
| | % Indels | Mean | N/A | 2.30 | 0.40 | 0.05 | 0.01 | 0.01 | N/A |
| | | SD | N/A | 1.50 | 0.01 | 0.05 | 0.00 | 0.00 | N/A |
| nCas9-CfaN-T2A- | % Templated | Mean | N/A | 76.50 | 32.14 | 3.63 | 0.27 | 0.01 | N/A |
| Vpx + CfaC-PolK(1- | Edits | SD | N/A | 7.33 | 4.06 | 1.09 | 0.06 | 0.01 | N/A |
| 526, GE411- | % Templated + | Mean | N/A | 1.38 | 0.45 | 0.05 | 0.00 | 0.00 | N/A |
| 412RV) + FEN1 | Byproduct Edits | SD | N/A | 0.81 | 0.11 | 0.05 | 0.00 | 0.00 | N/A |
| | % Indels | Mean | N/A | 1.00 | 0.25 | 0.02 | 0.00 | 0.00 | N/A |
| | | SD | N/A | 0.69 | 0.16 | 0.02 | 0.00 | 0.00 | N/A |
| nCas9-CfaN-T2A- | % Templated | Mean | N/A | 77.19 | 29.43 | 3.21 | 0.18 | 0.00 | N/A |
| Vpx + CfaC- | Edits | SD | N/A | 5.11 | 6.95 | 1.77 | 0.04 | 0.00 | N/A |
| polKvariant-FEN1 | % Templated + | Mean | N/A | 1.48 | 0.38 | 0.02 | 0.00 | 0.00 | N/A |
| | Byproduct Edits | SD | N/A | 0.54 | 0.08 | 0.02 | 0.00 | 0.00 | N/A |
| | % Indels | Mean | N/A | 0.68 | 0.35 | 0.00 | 0.01 | 0.00 | N/A |
| | | SD | N/A | 0.21 | 0.13 | 0.00 | 0.01 | 0.00 | N/A |
| HMGB1dT-nCas9- | % Templated | Mean | N/A | 84.75 | 61.95 | 17.11 | 0.92* | 0.07* | N/A |
| CfaN + CfaC-PolK(1- | Edits | SD | N/A | 9.25 | 6.22 | 1.68 | N/A | N/A | N/A |
| 526, GE411-412RV) | % Templated + | Mean | N/A | 1.77 | 0.76 | 0.19 | 0.02* | 0.00* | N/A |
| | Byproduct Edits | SD | N/A | 0.33 | 0.10 | 0.01 | N/A | N/A | N/A |
| | % Indels | Mean | N/A | 1.24 | 0.52 | 0.09 | 0.00* | 0.00* | N/A |
| | | SD | N/A | 0.34 | 0.14 | 0.07 | N/A | N/A | N/A |
| HMGB1dT-nCas9- | % Templated | Mean | N/A | 86.41 | 53.32 | 9.82 | 0.96 | 0.17* | N/A |
| CfaN + CfaC-PolK(1- | Edits | SD | N/A | 3.85 | 5.27 | 7.04 | 0.03 | N/A | N/A |
| 526, GE411- | % Templated + | Mean | N/A | 1.81 | 0.97 | 0.12 | 0.01 | 0.00* | N/A |
| 412RV) + FEN1 | Byproduct Edits | SD | N/A | 0.08 | 0.14 | 0.01 | 0.01 | N/A | N/A |
| | % Indels | Mean | N/A | 0.89 | 0.80 | 0.08 | 0.01 | 0.00* | N/A |
| | | SD | N/A | 0.08 | 0.36 | 0.10 | 0.01 | N/A | N/A |
| HMGB1dT-nCas9- | % Templated | Mean | 94.24* | 86.40 | 54.76 | 11.37 | 1.10 | 0.10* | 0.05* |
| CfaN + CfaC-PolK(1- | Edits | SD | N/A | 1.40 | 9.45 | 2.02 | 0.62 | N/A | N/A |
| 526, GE411- | % Templated + | Mean | 1.78* | 1.83 | 0.95 | 0.14 | 0.01 | 0.00* | 0.00* |
| 412RV) + FEN1 + Vpx | Byproduct Edits | SD | N/A | 0.05 | 0.21 | 0.05 | 0.01 | N/A | N/A |
| | % Indels | Mean | 2.48* | 1.08 | 0.73 | 0.10 | 0.01 | 0.00* | 0.00* |
| | | SD | N/A | 0.27 | 0.14 | 0.07 | 0.01 | N/A | N/A |
| HMGB1dT-nCas9- | % Templated | Mean | 86.74* | 77.89 | 36.19 | 6.44 | 0.46 | 0.00* | 0.00* |
| CfaN + CfaC- | Edits | SD | N/A | 3.34 | 3.95 | 0.52 | 0.25 | N/A | N/A |
| polKvariant-FEN1 | % Templated + | Mean | 2.09* | 1.29 | 1.55 | 0.03 | 0.04 | 0.00* | 0.00* |
| | Byproduct Edits | SD | N/A | 0.16 | 1.84 | 0.01 | 0.06 | N/A | N/A |
| | % Indels | Mean | 2.20* | 1.07 | 0.43 | 0.01 | 0.04 | 0.01* | 0.00* |
| | | SD | N/A | 0.13 | 0.29 | 0.01 | 0.06 | N/A | N/A |
| HMGB1dT-nCas9- | % Templated | Mean | N/A | 69.99* | 33.08 | 6.17 | 0.50 | 0.02 | N/A |
| CfaN-T2A-Vpx + CfaC- | Edits | SD | N/A | N/A | 8.36 | 1.90 | 0.71 | 0.03 | N/A |
| PolK(1-526, GE411- | % Templated + | Mean | N/A | 0.68* | 0.36 | 0.05 | 0.01 | 0.00 | N/A |
| 412RV) | Byproduct Edits | SD | N/A | N/A | 0.30 | 0.01 | 0.01 | 0.00 | N/A |
| | % Indels | Mean | N/A | 0.68* | 0.15 | 0.03 | 0.01 | 0.00 | N/A |
| | | SD | N/A | N/A | 0.12 | 0.03 | 0.01 | 0.00 | N/A |
| HMGB1dT-nCas9- | % Templated | Mean | 89.13* | 70.93 | 26.74 | 7.81 | 0.77 | 0.01* | 0.00* |
| CfaN-T2A-Vpx + CfaC- | Edits | SD | N/A | 5.11 | 1.17 | 3.97 | 0.14 | N/A | N/A |
| PolK(1-526, GE411- | % Templated + | Mean | 2.51* | 1.01 | 0.68 | 0.04 | 0.00 | 0.00* | 0.00* |
| 412RV) + FEN1 | Byproduct Edits | SD | N/A | 0.30 | 0.08 | 0.02 | 0.00 | N/A | N/A |
| | % Indels | Mean | 1.15* | 0.55 | 0.24 | 0.15 | 0.00 | 0.01* | 0.00* |
| | | SD | N/A | 0.18 | 0.34 | 0.20 | 0.00 | N/A | N/A |
| HMGB1dT-nCas9- | % Templated | Mean | N/A | 66.76 | 14.70 | 3.62 | 0.02 | 0.00* | 0.16* |
| CfaN-T2A-Vpx + CfaC- | Edits | SD | N/A | 5.54 | 2.88 | 1.32 | 0.03 | N/A | N/A |
| polKvariant-FEN1 | % Templated + | Mean | N/A | 1.10 | 0.11 | 0.04 | 0.00 | 0.00* | 0.00* |
| | Byproduct Edits | SD | N/A | 0.30 | 0.08 | 0.00 | 0.00 | N/A | N/A |
| | % Indels | Mean | N/A | 0.65 | 0.06 | 0.03 | 0.01 | 0.01* | 0.00* |
| | | SD | N/A | 0.22 | 0.08 | 0.02 | 0.01 | N/A | N/A |

*N = 1

### Example 48—Editing at the TTR Locus in PCH Using Various Constructs Comprising Split Inteins

This Example evaluates the editing efficiency of a SpyCas9 H840A nickase (nCas9) delivered in trans with a polymerase, using various pairs of inteins. The following split-inteins are assessed: (i) M86-N2 and M86-C2; (ii) NpuSsp-N3 and NpuSsp-C3, (iii) gp41-1-N2 and gp41-1-C₂, (iv) gp41-8-N2 and gp41-8-C2, (v) NrdJ-1-N2 and NrdJ-1-C₂, (vi) IMPDH-1-N2 and IMPDH-1-C₂, (vii) SspDnaX-N2 and SspDnaX-C2, (viii) SspGyrB-N2 and SspGyrB-C2; (ix) Cth-Ter-N2 and Cth-Ter-C2; (x) NrdA-2-N2 and NrdA-2-C₂; (xi) Mja-KlbA-N2 and Mja-KlbA-C2; (xii) RBS1N and RBS1C; (xiii) TE3S11N and TE3S11C; and (xiv) RmaN and RmaC.

A SpyCas9 H840A nickase (nCas9) and a polymerase Kappa variant are delivered into primary cynomolgus hepatocytes (PCH) in trans, along a mRNA encoding Vpx, and a tgRNA (G036237) comprising a spacer sequence targeting the TTR locus, a scaffold with the structure shown in FIG. 6D, and a 3′ extension comprising a template encoding a TG-to-AC edit, resulting in a STOP codon, and a DRS. The nCas9 and polymerase constructs, as well as the corresponding intein variants are listed in Table 98.

PCH (Gibco, Lot CY423) are prepared as described in Example 1, and plated on a collagen I coated 96-well plate (Stem cell technologies, Cat. 100-0366) at a density of 40,000 cells/well. PCH are lipofected using pre-mixed lipid formulations with a molar composition of 50% Lipid A, 38% cholesterol, 9% DSPC, and 3% PEG2k-DMG as described in Example 1. Lipofections are performed with a tgRNA (G036237) at a fixed dose of 25 nM, and a mRNA load in a 5-point 2-fold dilution series starting at 400 ng total mRNA. Cells are either treated with an mRNA encoding a full-length nCas9-PolKv2 fusion protein (SEQ ID NO: 1371), or with a combination of a mRNA with an ORF encoding a nCas9-inteinN and a mRNA encoding a corresponding inteinC-polymerase as listed in Table 98.

TABLE 98
List of ORFs encoding exemplary split
intein constructs used herein.

| SEQ ID NO: | Constructs name | Intein Variants |
|---|---|---|
| 1411 | nCas9-M86-N2 | M86-N2 |
| 1413 | M86-C2-polK | M86-C2 |
| 1415 | nCas9-NpuSsp-N3 | NpuSsp-N3 |
| 1417 | NpuSsp-C3-polK | NpuSsp-C3 |
| 1419 | nCas9-gp41-1-N2 | gp41-1-N2 |
| 1421 | gp41-1-C2-polK | gp41-1-C2 |
| 1423 | nCas9-gp41-8-N2 | gp41-8-N2 |
| 1425 | gp41-8-C2-polK | gp41-8-C2 |
| 1427 | nCas9-NrdJ-1-N2 | NrdJ-1-N2 |
| 1429 | NrdJ-1-C2-polK | NrdJ-1-C2 |
| 1431 | nCas9-IMPDH-1-N2 | IMPDH-1-N2 |
| 1433 | IMPDH-1-C2-polK | IMPDH-1-C2 |
| 1435 | nCas9-SspDnaX-N2 | SspDnaX-N2 |
| 1437 | SspDnaX-C2-polK | SspDnaX-C2 |
| 1439 | nCas9-SspGyrB-N2 | SspGyrB-N2 |
| 1441 | SspGyrB-C2-polK | SspGyrB-C2 |
| 1443 | nCas9-Cth-Ter-N2 | Cth-Ter-N2 |
| 1389 | Cth-Ter-C2-polK | Cth-Ter-C2 |
| 1391 | nCas9-NrdA-2-N2 | NrdA-2-N2 |
| 1393 | NrdA-2-C2-polK | NrdA-2-C2 |
| 1395 | nCas9-Mja-KlbA-N2 | Mja-KlbA-N2 |
| 1397 | Mja-KlbA-C2-polK | Mja-KlbA-C2 |
| 1399 | nCas9-RBS1N | RBS1N |
| 1401 | RBS1C-polK | RBS1C |
| 1403 | nCas9-TE3S11N | TE3S11N |
| 1405 | TE3S11C-polK | TE3S11C |
| 1407 | nCas9-RmaN | RmaN |
| 1409 | RmaC-polK | RmaC |

Cells are incubated at 37° C., 5% CO₂ for 24 hours prior to treatment with the pre-mixed lipid formulations. The cells are lysed 72 hours post-treatment for NGS analysis as described in Example 1.

### Example 49—Editing at the HEK3 Locus in PHH Using nCas9-Polymerase Fusion Proteins Comprising a HMGB1 Sequence

This Example evaluated the impact of a chromatin remodeler (HMGB1) on templated editing efficiency. Fusion proteins comprising a SpyCas9 nickase (nCas9) moiety, a polymerase moiety and a HMGB1 moiety were evaluated for editing efficiency at the HEK3 locus in primary human hepatocytes (PHH).

PHH (Gibco, Lot 8381) were prepared as described in Example 1, and plated on a collagen I coated 96-well plate (Stem cell technologies, Cat. 100-0366) at a density of 35,000 cells/well. PHH were lipofected using pre-mixed lipid formulations with a molar composition of 50% Lipid A, 38% cholesterol, 9% DSPC, and 3% PEG2k-DMG as described in Example 1. Lipofections were performed with 25 nM tgRNA (G032574, with the scaffold structure shown in FIG. 6E, and a template encoding a G-to-C edit), 50 ng/well of Vpx (SEQ ID NO: 1501), and 50 ng/well of a mRNA encoding a fusion protein selected from: nCas9-PolK (1-526, GE411-412RV) (SEQ ID NO: 1232), HMGB1dT-nCas9-PolK (1-526, GE411-412RV) (SEQ ID NO: 1445), nCas9-T5pol(v2) (SEQ ID NO: 1217), and HMGBldT-nCas9-T5pol(v2) (SEQ ID NO: 1448). The cells were lysed 72 hours post-treatment for NGS analysis as described in Example 1. Samples were run in triplicates, unless otherwise indicated.

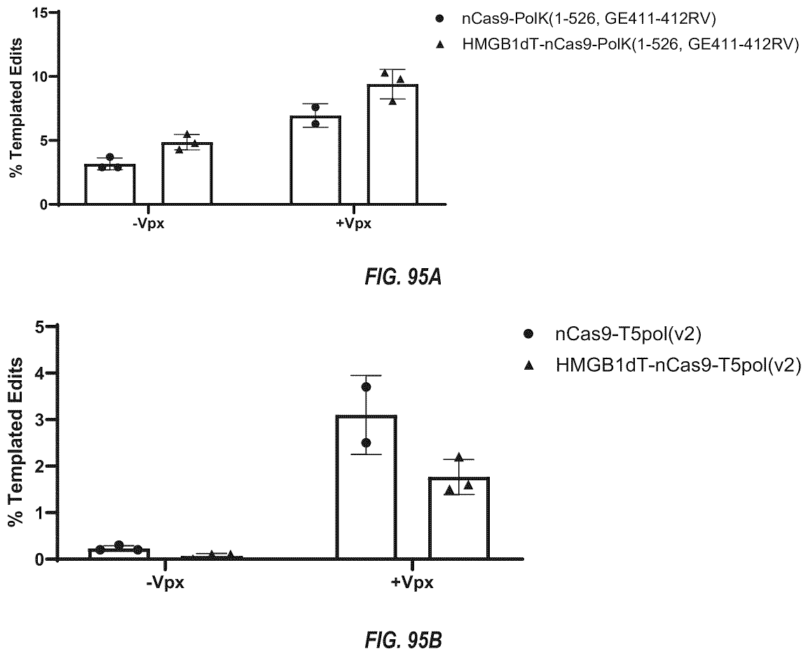

Table 99 and FIGS. 95A-B show the editing results at the HEK3 locus in PHH for cells treated with G032574, along with nCas9-PolK (1-526, GE411-412RV) (SEQ ID NO: 1232), HMGB1dT-nCas9-PolK (1-526, GE411-412RV) (SEQ ID NO: 1445), nCas9-T5pol(v2) (SEQ ID NO: 1217), or HMGB1dT-nCas9-T5pol(v2) (SEQ ID NO: 1448), in the absence or presence of Vpx.

TABLE 99
Editing at the HEK3 locus in PHH for cells treated with G032574,
along with nCas9-PolK(1-526, GE411-412RV) (SEQ ID NO: 1232),
HMGB1dT-nCas9-PolK(1-526, GE411-412RV) (SEQ ID NO: 1445),
nCas9-T5pol(v2) (SEQ ID NO: 1217), or HMGB1dT-nCas9-T5pol(v2)
(SEQ ID NO: 1448), in the absence or presence of Vpx.

| | | % Templated | | % Templated + | | | |
|---|---|---|---|---|---|---|---|
| | Editing | Edits | | Byproduct Edits | | % Indels | |
| nCas9-polymerase mRNA | Enhancer | Mean | SD | Mean | SD | Mean | SD |
| nCas9-PolK(1-526, GE411- | none | 3.17 | 0.46 | 0.27 | 0.06 | 0.00 | 0.00 |
| 412RV) | Vpx | 6.95* | 0.92* | 0.65* | 0.07* | 0.10* | 0.14* |
| HMGB1dT-nCas9-PolK(1- | none | 4.87 | 0.60 | 0.33 | 0.06 | 0.07 | 0.06 |
| 526, GE411-412RV) | Vpx | 9.40 | 1.15 | 0.83 | 0.21 | 0.13 | 0.06 |
| nCas9-T5pol(v2) | none | 0.23 | 0.06 | 0.00 | 0.00 | 0.00 | 0.00 |
| | Vpx | 3.10* | 0.85* | 0.20* | 0.14* | 0.00* | 0.00* |
| HMGB1dT-nCas9- | none | 0.07 | 0.06 | 0.00 | 0.00 | 0.00 | 0.00 |
| T5pol(v2) | Vpx | 1.77 | 0.38 | 0.13 | 0.12 | 0.07 | 0.06 |

*N = 2

### Example 50—Editing at the TTR Locus in PCH Using Constricts Comprising a SpyCas9 Nickase, a Polymerase Kappa Variant, and HMGB1 Operably Linked to Split Inteins

The Example evaluates the impact of a chromatin remodeler (e.g., HMGB1) on templated editing efficiency in the context of a SpyCas9 nickase and a DNA Polymerase delivered in trans via a split-intein system. The editing efficiency of a fusion protein comprising a SpyCas9 nickase (nCas9) moiety, a HMGB1 variant moiety and a first split intein, delivered along with a fusion protein comprising a polymerase moiety and a second, complementary split intein capable of undergoing a trans-splicing reaction with the first split intein, is evaluated in primary cynomolgus hepatocytes (PCH) in the presence of editing enhancers delivered in trans or in cis with the polymerase. A tgRNA (G036237) is also delivered to the cells, wherein the tgRNA comprises a spacer sequence targeting the TTR locus, a scaffold with the structure shown in FIG. 6D, and a 3′ extension comprising a template encoding a TG-to-AC edit, resulting in a STOP codon, and a DRS. FEN1 or Vpx are either fused to the polymerase, or are delivered in trans.

PCH (Gibco, Lot CY423) are prepared as described in Example 1, and plated on a collagen I coated 96-well plate (Stem cell technologies, Cat. 100-0366) at a density of 40,000 cells/well. PCH are lipofected using pre-mixed lipid formulations with a molar composition of 50% Lipid A, 38% cholesterol, 9% DSPC, and 3% PEG2k-DMG as described in Example 1. Lipofections are performed with a tgRNA (G036237) at a fixed dose of 25 nM, and a mRNA load in a 5-point 2-fold dilution series starting at 400 ng total mRNA. Cells are either treated with an mRNA encoding a full-length HMGBldT-nCas9-polymerase fusion protein (comprising, in some groups, an EI coil at its N-terminal) and one or more mRNA(s) encoding a Vpx or a FEN1 editing enhancer, or with a combination of a mRNA comprising a nCas9-inteinN sequence and a Vpx sequence separated by a T2A domain (and comprising, in some groups, a HMGB1 sequence) and a mRNA comprising a corresponding inteinC-polymerase sequence and a FEN1 or Vpx sequence (and comprising, in some groups, a EI coil or a KI coil) as listed in Table 100.

TABLE 100
Exemplary constructs used herein.

| SEQ | |
|---|---|
| ID | |
| NO: | Construct composition |
| 1295 | CfaC-linker-SV40NLS-polKvariant-X10linker-EIcoil-T2A- |
| | FEN1-KIcoil-P2A-VPX |
| 1292 | CfaC-linker-SV40NLS-polKvariant-linker-SV40NLS-T2A-VPX- |
| | P2A-FEN1 |
| 1289 | CfaC-linker-SV40NLS-polKvariant-linker-SV40NLS-T2A- |
| | FEN1-P2A-VPX |
| 1286 | CfaC-linker-SV40NLS-polKvariant-linker-SV40NLS-X10linker- |
| | FEN1-P2A-VPX |
| 1283 | CfaC-linker-SV40NLS-polKvariant-linker-SV40NLS-X10linker- |
| | FEN1 |
| 1280 | NucleoplasminNLS-X10linker-HMGBdT-linker-nCas9-linker- |
| | CfaN-T2A-VPX |
| 1277 | NucleoplasminNLS-nCas9-linker-CfaN-T2A-VPX |
| 1451 | NucleoplasminNLS-X10linker-HMGBdT-linker-nCas9-linker- |
| | SV40NLS-polKvariant-X10linker-SV40NLS-X10linker-EIcoil |
| 1298 | NucleoplasminNLS-X10linker-HMGBdT-linker-nCas9-linker- |
| | SV40NLS-polKvariant-linker-SV40NLS |
| 1445 | SV40NLS-X10linker-HMGB1dT-linker-nCas9- linker- |
| | polKvariant-SV40NLS |

Cells are incubated at 37° C., 5% CO₂ for 24 hours prior to treatment with the pre-mixed lipid formulations. The cells are lysed 72 hours post-treatment for NGS analysis as described in Example 1.

### Example 51—Editing at the VEGFA Locus in Huh7 Cells Using tgRNAs with Modified Nucleotides at the Template:DRS Junction

In this Example, templated editing efficiency was assessed with tgRNAs having different RNA/DNA compositions at the junction between the template sequence and DNA polymerase recruiting sequence (DRS).

Editing efficiency was assessed at the VEGFA locus in huh7 cells when tgRNAs with different rNTP/dNTP compositions at the template:DRS junction were used along with a mRNA encoding a nCas9-polymerase fusion protein.

Huh7 cells were prepared as described in Example 1, and plated in 96-well plates (Falcon, 353072) at a density of 15,000 cells/well. Lipofectamine MessengerMAX (ThermoFisher Scientific, LMRNA015) lipoplexes were prepared according to manufacturer's instructions, with 10 ng/well of tgRNA and 100 ng/well of mRNA encoding a SpyCas9 nickase fused to one of the following moieties: a polymerase Kappa variant (nCas9-PolKv2) (SEQ ID NO: 1371), a wild-type T5 polymerase (nCas9-T5(WT)) (SEQ ID NO: 1301), or a T5 polymerase variant (nCas9-T5(D164A, E166A)) (SEQ ID NO: 1304). The tgRNAs comprised a spacer targeting the VEGFA locus, a scaffold with the structure shown in FIG. 6D, a template sequence encoding a GAGG-to-CACT edit, and a DRS sequence. The tgRNAs differed in the dNTP/rNTP composition at the template:DRS junction, as shown in Table 111.

TABLE 111
Exemplary tgRNAs with various dNTP/rNTP compositions at the template:DRS junction.

| Guide ID | Description | 3′extension sequence (template sequence is shown in bolded italics) |
|---|---|---|
| TG039605 | rNTPs at | dTdGdCdCdAdTdCdTdGdGdAdGdCdAdGdTGAUCUGGmCmCmUmG |
| (SEQ ID | T1-T2 | mCmA*mG*mU (SEQ ID NO: 277) |
| NO: 543) | | |
| TG039606 | rNTP at T1 | dTdGdCdCdAdTdCdTdGdGdAdGdCdAdGdTdGAUCUGGmCmCmUm |
| (SEQ ID | | GmCmA*mG*mU (SEQ ID NO: 278) |
| NO: 544) | | |
| TG040924 | dNTPs in | dTdGdCdCdAdTdCdTdGdGdAdGdCdAdGdTdGdAUCUGGmCmCmU |
| (SEQ ID | template, | mGmCmA*mG*mU (SEQ ID NO: 279) |
| NO: 545) | rNTPs in | |
| | DRS | |
| TG039607 | dNTP at D1 | dTdGdCdCdAdTdCdTdGdGdAdGdCdAdGdTdGdAdTCUGGmCmCmU |
| (SEQ ID | | mGmCmA*mG*mU (SEQ ID NO: 280) |
| NO: 546) | | |
| TG039608 | dNTPs at | dTdGdCdCdAdTdCdTdGdGdAdGdCdAdGdTdGdAdTdCUGGmCmCm |
| (SEQ ID | D1-D2 | UmGmCmA*mG*mU (SEQ ID NO: 281) |
| NO: 547) | | |
| TG039609 | dNTPs at | dTdGdCdCdAdTdCdTdGdGdAdGdCdAdGdTdGdAdTdCdTGGmCmCm |
| (SEQ ID | D1-D3 | UmGmCmA*mG*mU (SEQ ID NO: 282) |
| NO: 548) | | |
| TG039610 | dNTPs at | dTdGdCdCdAdTdCdTdGdGdAdGdCdAdGdTdGdAdTdCdTdGGmCmC |
| (SEQ ID | D1-D4 | mUmGmCmA*mG*mU (SEQ ID NO: 283) |
| NO: 549) | | |
| TG039611 | dNTPs at | dTdGdCdCdAdTdCdTdGdGdAdGdCdAdGdTdGdAdTdCdTdGdGmCm |
| (SEQ ID | D1-D5 | CmUmGmCmA*mG*mU (SEQ ID NO: 284) |
| NO: 550) | | |
| TG039612 | dNTPs at | dTdGdCdCdAdTdCdTdGdGdAdGdCdAdGdTdGdAdTCdTGdGmCmCm |
| (SEQ ID | D1,D3,D5 | UmGmCmA*mG*mU (SEQ ID NO: 285) |
| NO: 551) | | |

Note:
*denotes that the nucleotide is linked to the next nucleotide with a phosphorothioate (PS bond), a lower case “m” denotes that the nucleotide is modified with 2′-O-Me, and a lowercase “d” denotes that the nucleotide is a DNA nucleotide.

The cells were lysed 72 hours post-treatment for NGS analysis as described in Example 1. Samples were run in triplicates, unless otherwise indicated.

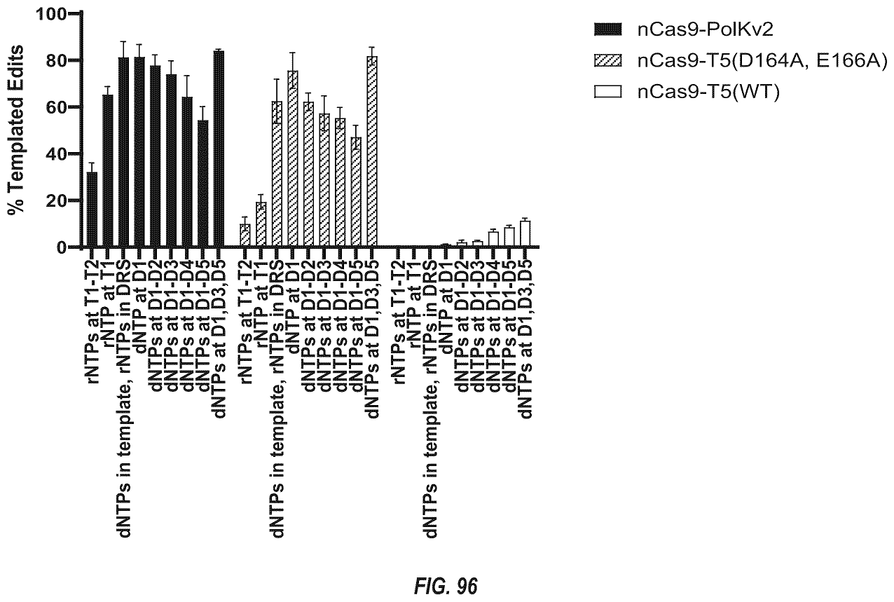

Table 112 and FIG. 96 show the editing results at the VEGFA locus in huh7 cells treated with tgRNAs comprising different dNTP/rNTP compositions at the template:DRS junction.

TABLE 112
Editing at the VEGFA locus in huh7 cells treated with a mRNA encoding a nCas9-polymerase fusion protein
selected from nCas9-PolKv2, nCas9-T5(WT), or nCas9-T5(D164A, E166A), and with tgRNAs encoding a GAGG-
to-CACT edit, and comprising different dNTP/rNTP compositions at the template: DRS junction.

| | | | Guide description | | | | | | | | |
|---|---|---|---|---|---|---|---|---|---|---|---|
| | | | rNTPs | | dNTPs in | | dNTPs | dNTPs | dNTPs | dNTPs | dNTPs |
| | | | at T1- | rNTP | template, | dNTP at | at D1- | at D1- | at D1- | at D1- | at D1, |
| | | | T2 | at T1 | rNTPs in DRS | D1 | D2 | D3 | D4 | D5 | D3, D5 |
| nCas9- | % | Mean | 32.19 | 65.30 | 81.29 | 81.45 | 77.78 | 74.06 | 64.40 | 54.37 | 84.10 |
| PolKv2 | Templated | SD | 3.94 | 3.51 | 6.72 | 5.39 | 4.64 | 5.74 | 9.08 | 5.83 | 0.71 |
| | Edits | | | | | | | | | | |
| | % | Mean | 0.49 | 1.24 | 0.78 | 1.27 | 1.60 | 1.31 | 1.11 | 1.20 | 1.20 |
| | Templated + | SD | 0.30 | 0.22 | 0.10 | 0.33 | 0.10 | 0.19 | 0.61 | 0.20 | 0.20 |
| | Byproduct | | | | | | | | | | |
| | Edits | | | | | | | | | | |
| | % Indels | Mean | 0.48 | 0.75 | 1.04 | 1.03 | 1.43 | 1.14 | 1.07 | 0.83 | 1.41 |
| | | SD | 0.26 | 0.20 | 0.07 | 0.33 | 0.32 | 0.38 | 0.47 | 0.23 | 0.11 |
| nCas9- | % | Mean | 9.98 | 19.40 | 62.52 | 75.65 | 62.26 | 57.33 | 55.29 | 47.08 | 81.80 |
| T5(D164A, | Templated | SD | 2.97 | 3.10 | 9.46 | 7.64 | 3.75 | 7.49 | 4.60 | 5.16 | 3.80 |
| E166A) | Edits | | | | | | | | | | |
| | % | Mean | 0.18 | 0.29 | 0.58 | 0.65 | 0.98 | 0.81 | 1.02 | 0.95 | 0.80 |
| | Templated + | SD | 0.15 | 0.07 | 0.12 | 0.17 | 0.23 | 0.19 | 0.16 | 0.35 | 0.14 |
| | Byproduct | | | | | | | | | | |
| | Edits | | | | | | | | | | |
| | % Indels | Mean | 0.73 | 0.96 | 0.80 | 1.01 | 1.39 | 1.08 | 0.98 | 1.08 | 1.29 |
| | | SD | 0.43 | 0.20 | 0.37 | 0.35 | 0.14 | 0.40 | 0.38 | 0.44 | 0.25 |
| nCas9- | % | Mean | 0.09 | 0.11 | 0.29 | 1.21 | 2.14 | 2.62 | 6.72 | 8.51 | 11.36 |
| T5(WT) | Templated | SD | 0.06 | 0.06 | 0.04 | 0.15 | 0.90 | 0.31 | 1.00 | 0.86 | 1.06 |
| | Edits | | | | | | | | | | |
| | % | Mean | 0.19 | 0.21 | 0.20 | 0.21 | 0.13 | 0.21 | 0.15 | 0.35 | 0.58 |
| | Templated + | SD | 0.16 | 0.21 | 0.04 | 0.08 | 0.11 | 0.14 | 0.14 | 0.12 | 0.03 |
| | Byproduct | | | | | | | | | | |
| | Edits | | | | | | | | | | |
| | % Indels | Mean | 5.74 | 7.32 | 6.51 | 7.16 | 7.20 | 6.09 | 3.80 | 3.47 | 7.73 |
| | | SD | 1.87 | 1.46 | 1.46 | 1.34 | 1.54 | 0.95 | 1.08 | 0.98 | 1.14 |

### Example 52—Editing at the VEGFA Locus in Huh7 Cells Using tgRNAs with Modified Nucleotides at Various Positions in the DRS Region

In this Example, templated editing efficiency was assessed with tgRNAs having various RNA/DNA compositions in the DNA polymerase recruiting sequence (DRS).

Editing efficiency was assessed at the VEGFA locus in huh7 cells when tgRNAs with different rNTP/dNTP compositions in the DRS were used along with a mRNA encoding a nCas9-polymerase fusion protein.

Huh7 cells were prepared as described in Example 1, and plated in 96-well plates (Falcon, 353072) at a density of 15,000 cells/well. Lipofectamine MessengerMAX (ThermoFisher Scientific, LMRNA015) lipoplexes were prepared according to manufacturer's instructions, with 10 ng/well of tgRNA and 100 ng/well of mRNA encoding nCas9-PolKv2 (SEQ ID NO: 1371) or nCas9-T5(D164A, E166A) (SEQ ID NO: 1304). The tgRNAs comprised a spacer targeting the VEGFA locus, a scaffold, a template sequence encoding a GAGG-to-CACT edit, and a DRS sequence. The tgRNAs differed in the dNTP/rNTP composition of the DRS, as shown in Table 113.

TABLE 113
Exemplary tgRNAs with various dNTP/rNTP compositions in the DRS.

| | dNTP positions in | |
|---|---|---|
| Guide ID | the DRS region | DRS sequence |
| TG039613 (SEQ ID | D1, D6-D12 | dTCUGGdCdCdTdGdCdA*dG*mU (SEQ ID |
| NO: 552) | | NO: 942) |
| TG039614 (SEQ ID | D1, D6-D8, D10-D12 | dTCUGGdCdCdTmGdCdA*dG*mU (SEQ ID |
| NO: 553) | | NO: 943) |
| TG039615 (SEQ ID | D1, D6-D7, D9-D10, D12 | dTCUGGdCdCmUdGdCmA*dG*mU (SEQ ID |
| NO: 554) | | NO: 944) |
| TG039617 (SEQ ID | D1, D7, D9, D11 | dTCUGGmCdCmUdGmCdA*mG*mU (SEQ ID |
| NO: 556) | | NO: 945) |
| TG039618 (SEQ ID | D1, D8, D11 | dTCUGGmCmCdTmGmCdA*mG*mU (SEQ ID |
| NO: 557) | | NO: 946) |
| TG039619 (SEQ ID | D1, D6, D9, D12 | dTCUGGdCmCmUdGmCmA*dG*mU (SEQ ID |
| NO: 558) | | NO: 947) |
| TG039620 (SEQ ID | D1, D6-D7, D10 | dTCUGGdCdCmUdGdCmA*mG*mU (SEQ ID |
| NO: 559) | | NO: 948) |
| TG039621 (SEQ ID | D1, D6-D7, D10-D11 | dTCUGGdCdCmUdGdCdA*mG*mU (SEQ ID |
| NO: 560) | | NO: 949) |
| TG039622 (SEQ ID | D1, D8-D9, D12 | dTCUGGmCmCdTdGmCmA*dG*mU (SEQ ID |
| NO: 561) | | NO: 950) |
| TG039623 (SEQ ID | D1, D9 | dTCUGGmCmCmUdGmCmA*mG*mU (SEQ |
| NO: 562) | | ID NO: 951) |
| TG039607 (SEQ ID | D1 | dTCUGGmCmCmUmGmCmA*mG*mU (SEQ |
| NO: 546) | | ID NO: 952) |
| TG039624 (SEQ ID | D1, D3, D5-D12 | dTCdTGdGdCdCdTdGdCdA*dG*mU (SEQ ID |
| NO: 563) | | NO: 953) |
| TG039625 (SEQ ID | D1, D3, D5-D8, D10-D12 | dTCdTGdGdCdCdTmGdCdA*dG*mU (SEQ ID |
| NO: 564) | | NO: 954) |
| TG039626 (SEQ ID | D1, D3, D5-D7, D9-D10, | dTCdTGdGdCdCm UdGdCmA*dG*mU (SEQ |
| NO: 565) | D12 | ID NO: 955) |
| TG039628 (SEQ ID | D1, D3, D5, D7, D9, D11 | dTCdTGdGmCdCmUdGmCdA*mG*mU (SEQ |
| NO: 567) | | ID NO: 956) |
| TG039629 (SEQ ID | D1, D3, D5, D8, D11 | dTCdTGdGmCmCdTmGmCdA*mG*mU (SEQ |
| NO: 568) | | ID NO: 957) |
| TG039630 (SEQ ID | D1, D3, D5-D6, D9, D12 | dTCdTGdGdCmCmUdGmCmA*dG*mU (SEQ |
| NO: 569) | | ID NO: 958) |
| TG039631 (SEQ ID | D1, D3, D5-D7, D10 | dTCdTGdGdCdCmUmGdCmA*mG*mU (SEQ |
| NO: 570) | | ID NO: 959) |
| TG039632 (SEQ ID | D1, D3, D5-D7, D10-D11 | dTCdTGdGdCdCmUmGdCdA*mG*mU (SEQ |
| NO: 571) | | ID NO: 960) |
| TG039633 (SEQ ID | D1, D3, D5, D8-D9, D12 | dTCdTGdGmCmCdTdGmCmA*dG*mU (SEQ |
| NO: 572) | | ID NO: 961) |
| TG039634 (SEQ ID | D1, D3, D5, D9 | dTCdTGdGmCmCmUdGmCmA*mG*mU (SEQ |
| NO: 573) | | ID NO: 962) |
| TG039612 (SEQ ID | D1, D3, D5 | dTCdTGdGmCmCmUmGmCmA*mG*mU |
| NO: 551) | | (SEQ ID NO: 963) |
| TG039635 (SEQ ID | D1-D13 | dTdCdTdGdGdCdCdTdGdCdA*dG*dT (SEQ ID |
| NO: 574) | | NO: 964) |
| TG039636 (SEQ ID | D1-D12 | dTdCdTdGdGdCdCdTdGdCdA*dG*mU (SEQ |
| NO: 575) | | ID NO: 965) |

Note:
*denotes that the nucleotide is linked to the next nucleotide with a phosphorothioate (PS bond), a lower case “m” denotes that the nucleotide is modified with 2′-O-Me, and a lowercase “d” denotes that the nucleotide is a DNA nucleotide.

The cells were lysed 72 hours post-treatment for NGS analysis as described in Example 1. Samples were run in triplicates, unless otherwise indicated.

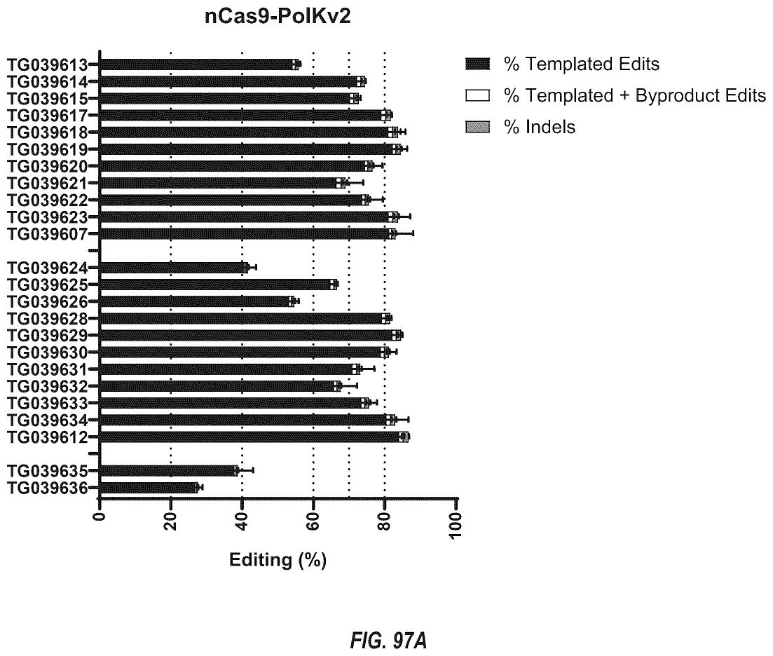

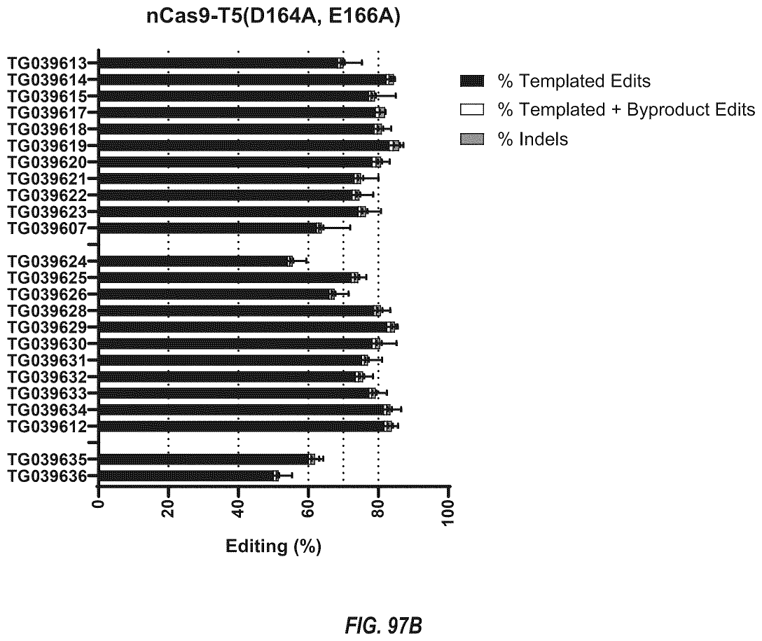

Table 114 and FIGS. 97A-B show the editing results at the VEGFA locus in huh7 cells treated with tgRNAs comprising different dNTP/rNTP compositions in the DRS region.

TABLE 114
Editing at the VEGFA locus in huh7 cells treated with a mRNA encoding a nCas9-polymerase
fusion (nCas9-PolKv2 or nCas9-T5(D164A, E166A)) and with tgRNAs encoding a GAGG-to-
CACT edit, and comprising different dNTP/rNTP compositions in the DRS region.

| | nCas9-PolKv2 | | | | | | nCas9-T5(D164A, E166A) | | | | | |
|---|---|---|---|---|---|---|---|---|---|---|---|---|
| | | | % Templated + | | | | % Templated | | % Templated + | | | |
| | % Templated Edits | | Byproduct Edits | | % Indels | | Edits | | Byproduct Edits | | % Indels | |
| Guide ID | Mean | SD | Mean | SD | Mean | SD | Mean | SD | Mean | SD | Mean | SD |
| TG039613 | 54.20 | 2.14 | 0.90 | 0.17 | 0.76 | 0.22 | 68.55 | 6.78 | 0.66 | 0.31 | 0.87 | 0.30 |
| TG039614 | 72.30 | 2.35 | 1.12 | 0.10 | 1.08 | 0.05 | 82.39 | 1.51 | 0.78 | 0.26 | 1.28 | 0.27 |
| TG039615 | 70.30 | 1.81 | 1.21 | 0.63 | 1.25 | 0.45 | 77.38 | 7.62 | 0.64 | 0.07 | 1.03 | 0.22 |
| TG039617 | 79.11 | 2.91 | 1.31 | 0.13 | 1.35 | 0.11 | 79.33 | 2.76 | 1.04 | 0.03 | 1.57 | 0.05 |
| TG039618 | 81.08 | 4.77 | 1.27 | 0.51 | 1.43 | 0.67 | 78.95 | 4.73 | 0.85 | 0.18 | 1.22 | 0.36 |
| TG039619 | 82.27 | 4.06 | 0.95 | 0.14 | 1.29 | 0.28 | 83.26 | 3.90 | 1.09 | 0.06 | 1.68 | 0.21 |
| TG039620 | 74.49 | 4.92 | 0.93 | 0.34 | 1.24 | 0.23 | 78.39 | 4.84 | 1.08 | 0.40 | 1.35 | 0.22 |
| TG039621 | 66.49 | 7.50 | 1.40 | 0.30 | 1.14 | 0.46 | 73.26 | 6.77 | 0.76 | 0.16 | 1.09 | 0.57 |
| TG039622 | 73.74 | 5.74 | 0.91 | 0.43 | 0.91 | 0.35 | 72.65 | 5.87 | 0.92 | 0.25 | 1.05 | 0.19 |
| TG039623 | 81.10 | 6.03 | 1.30 | 0.19 | 1.39 | 0.21 | 74.42 | 6.32 | 0.74 | 0.25 | 1.33 | 0.41 |
| TG039607 | 81.29 | 6.72 | 0.78 | 0.10 | 1.04 | 0.07 | 62.52 | 9.46 | 0.58 | 0.12 | 0.80 | 0.37 |
| TG039624 | 40.66 | 3.23 | 0.55 | 0.18 | 0.45 | 0.18 | 54.19 | 5.25 | 0.56 | 0.09 | 0.81 | 0.13 |
| TG039625 | 64.85 | 1.59 | 1.05 | 0.08 | 0.71 | 0.21 | 72.45 | 4.11 | 0.79 | 0.07 | 1.05 | 0.26 |
| TG039626 | 53.21 | 2.69 | 0.77 | 0.10 | 0.70 | 0.21 | 66.08 | 5.44 | 0.58 | 0.15 | 0.88 | 0.17 |
| TG039628 | 79.25 | 1.74 | 1.15 | 0.31 | 1.17 | 0.35 | 78.83 | 4.55 | 0.78 | 0.14 | 1.17 | 0.42 |
| TG039629 | 82.26 | 2.70 | 1.16 | 0.28 | 1.18 | 0.24 | 82.62 | 2.90 | 0.99 | 0.24 | 1.18 | 0.31 |
| TG039630 | 78.96 | 4.39 | 1.13 | 0.47 | 1.12 | 0.24 | 78.39 | 6.81 | 0.95 | 0.15 | 1.19 | 0.45 |
| TG039631 | 70.98 | 6.12 | 1.24 | 0.40 | 0.98 | 0.34 | 75.41 | 5.64 | 0.72 | 0.06 | 0.93 | 0.22 |
| TG039632 | 65.85 | 6.37 | 0.93 | 0.36 | 0.89 | 0.19 | 73.58 | 4.88 | 0.88 | 0.18 | 1.21 | 0.28 |
| TG039633 | 73.49 | 4.33 | 1.06 | 0.26 | 1.08 | 0.36 | 77.49 | 4.98 | 0.72 | 0.07 | 1.08 | 0.22 |
| TG039634 | 80.53 | 6.15 | 1.23 | 0.16 | 1.15 | 0.38 | 81.64 | 4.86 | 0.88 | 0.25 | 0.99 | 0.37 |
| TG039612 | 84.10 | 0.71 | 1.20 | 0.20 | 1.41 | 0.11 | 81.80 | 3.80 | 0.80 | 0.14 | 1.29 | 0.25 |
| TG039635 | 37.85 | 5.20 | 0.56 | 0.22 | 0.39 | 0.08 | 60.16 | 4.08 | 0.65 | 0.10 | 1.15 | 1.12 |
| TG039636 | 26.95 | 1.92 | 0.45 | 0.23 | 0.23 | 0.05 | 50.16 | 5.17 | 0.79 | 0.09 | 0.61 | 0.08 |

### Example 53—Editing at the VEGFA Locus in Huh7 Cells Using tgRNAs with Modified Nucleotides at Various Positions in the Spacer Sequence

In this Example, templated editing efficiency was assessed with tgRNAs having a spacer sequence with different DNA/RNA compositions or nucleotide modifications (e.g., 2′O-methyl(2′Ome) modifications) at various positions.

Editing efficiency was assessed at the VEGFA locus in huh7 cells when tgRNAs with different rNTP/dNTP compositions and different 2′Ome modifications in the spacer sequence were used along with a mRNA encoding an nCas9-polymerase fusion protein.

Huh7 cells were prepared as described in Example 1, and plated in 96-well plates (Falcon, 353072) at a density of 15,000 cells/well. Lipofectamine MessengerMAX (ThermoFisher Scientific, LMRNA015) lipoplexes were prepared according to manufacturer's instructions, with 10 ng/well of tgRNA and 100 ng/well of mRNA encoding a SpyCas9 nickase fused to one of the following moieties: a polymerase Kappa variant (nCas9-PolKv2) (SEQ ID NO: 1371), or a T5 polymerase variant (nCas9-T5(D164A, E166A)) (SEQ ID NO: 1304). The tgRNAs comprised a spacer targeting the VEGFA locus, a scaffold, a template sequence encoding a GAGG-to-CACT edit, and a DRS sequence. The tgRNAs differed in the dNTP/rNTP compositions and the 2′Ome modifications in the spacer sequence, as shown in Table 115.

TABLE 115
Exemplary tgRNAs with various dNTP/rNTP compositions and 2′Ome modifications in
the spacer sequence.

| | Modifications in the spacer | |
|---|---|---|
| Guide ID | sequence | Spacer sequence |
| TG039646 (SEQ | dNTPs at S4-S5, S9-S14, S17- | mG*mA*mU*dGdTCUGdCdAdGdGdCdCAG |
| ID NO: 576) | S18, S20 | dAdTGdA (SEQ ID NO: 969) |
| TG039645 (SEQ | dNTPs at S9-S14, S17-S18, | mG*mA*mU*GUCUGdCdAdGdGdCdCAGd |
| ID NO: 577) | S20 | AdTGdA (SEQ ID NO: 970) |
| TG039644 (SEQ | 2′Ome at S9, S13-S14, S20, | mG*mA*mU*dGdTCUGmCdAdGdGmCmC |
| ID NO: 578) | and dNTPs at S4-S5, S10-S12, | AGdAdTGmA (SEQ ID NO: 465) |
| | S17-S18 | |
| TG039643 (SEQ | 2′Ome at S9, S13-S14, S20, | mG*mA*mU*GUCUGmCdAdGdGmCmCAG |
| ID NO: 579) | and dNTPs at S10-S12, S17- | dAdTGmA (SEQ ID NO: 464) |
| | S18 | |
| TG039642 (SEQ | 2′Ome at S9-S10, S13-S14, | mG*mA*mU*GUCUGmCmAdGdGmCmCA |
| ID NO: 580) | S17, S20, and dNTPs at S11- | GmAdTGmA (SEQ ID NO: 968) |
| | S12, S18 | |
| TG039641 (SEQ | 2′Ome at S4-S5, S9-S14, S17- | mG*mA*mU*mGmUCUGmCmAmGmGmC |
| ID NO: 581) | S18, S20 | mCAGmAmUGmA (SEQ ID NO: 463) |
| TG039640 (SEQ | 2′Ome at S4-S5, S9, S13-S14, | mG*mA*mU*mGmUCUGmCAGGmCmCAG |
| ID NO: 582) | S20 | AUGmA (SEQ ID NO: 462) |
| TG039639 (SEQ | 2′Ome at S9-S14, S17-S18, | mG*mA*mU*GUCUGmCmAmGmGmCmC |
| ID NO: 583) | S20 | AGmAmUGmA (SEQ ID NO: 461) |
| TG039638 (SEQ | 2′Ome at S9-S10, S13-S14, | mG*mA*mU*GUCUGmCmAGGmCmCAGm |
| ID NO: 584) | S17, S20 | AUGmA (SEQ ID NO: 967) |
| TG039637 (SEQ | 2′Ome at S9, S13-S14, S20 | mG*mA*mU*GUCUGmCAGGmCmCAGAU |
| ID NO: 585) | | GmA (SEQ ID NO: 460) |
| TG039636 (SEQ | None (control) | mG*mA*mU*GUCUGCAGGCCAGAUGA |
| ID NO: 575) | | (SEQ ID NO: 966) |

Note:
*denotes that the nucleotide is linked to the next nucleotide with a phosphorothioate (PS bond), a lower case “m” denotes that the nucleotide is modified with 2′-O-Me, and a lowercase “d” denotes that the nucleotide is a DNA nucleotide.

The cells were lysed 72 hours post-treatment for NGS analysis as described in Example 1. Samples were run in triplicates, unless otherwise indicated.

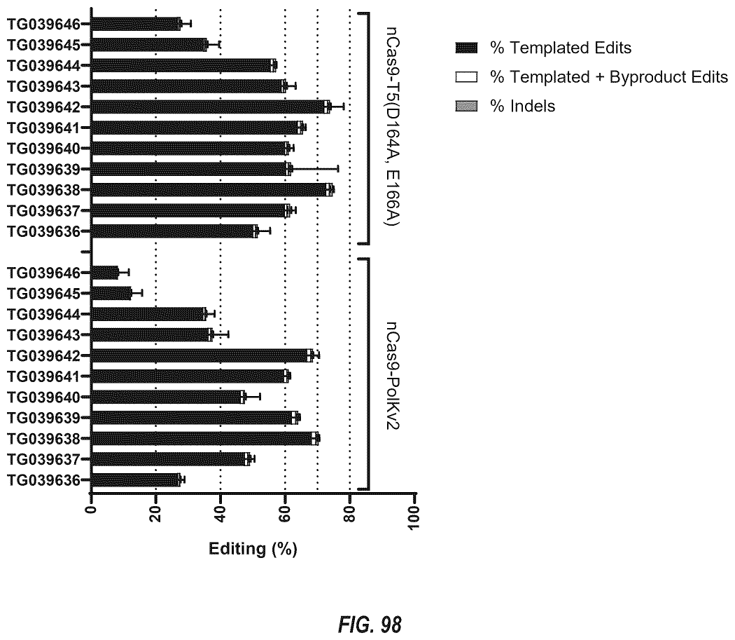

Table 116 and FIG. 98 show the editing results at the VEGFA locus in huh7 cells treated with tgRNAs comprising different dNTP/rNTP compositions and 2′Ome modifications in the spacer sequence.

TABLE 116
Editing at the VEGFA locus in huh7 cells treated with a mRNA encoding a nCas9-polymerase fusion
(nCas9-PolKv2 or nCas9-T5(D164A, E166A)) and with tgRNAs encoding a GAGG-to-CACT edit, and comprising
different dNTP/rNTP compositions and 2′Ome modifications in the spacer sequence.

| | nCas9-PolKv2 | | | | | | nCas9-T5(D164A, E166A) | | | | | |
|---|---|---|---|---|---|---|---|---|---|---|---|---|
| | % Templated | | % Templated + | | | | | | % Templated + | | | |
| | Edits | | Byproduct Edits | | % Indels | | % Templated Edits | | Byproduct Edits | | % Indels | |
| Guide ID | Mean | SD | Mean | SD | Mean | SD | Mean | SD | Mean | SD | Mean | SD |
| TG039646 | 8.00 | 3.65 | 0.20 | 0.11 | 0.09 | 0.06 | 26.80 | 4.03 | 0.45 | 0.29 | 0.40 | 0.30 |
| TG039645 | 11.92 | 3.85 | 0.32 | 0.10 | 0.08 | 0.08 | 34.90 | 4.65 | 0.42 | 0.08 | 0.45 | 0.26 |
| TG039644 | 34.68 | 3.54 | 0.64 | 0.08 | 0.28 | 0.08 | 55.56 | 1.02 | 0.88 | 0.18 | 0.76 | 0.18 |
| TG039643 | 36.30 | 6.12 | 0.75 | 0.27 | 0.53 | 0.17 | 58.97 | 4.26 | 0.60 | 0.09 | 0.72 | 0.22 |
| TG039642 | 66.84 | 3.67 | 1.18 | 0.22 | 0.59 | 0.09 | 72.17 | 5.96 | 1.01 | 0.14 | 0.74 | 0.22 |
| TG039641 | 59.67 | 1.95 | 0.96 | 0.21 | 0.47 | 0.21 | 63.98 | 2.36 | 0.91 | 0.30 | 0.64 | 0.03 |
| TG039640 | 46.30 | 5.90 | 0.88 | 0.14 | 0.40 | 0.24 | 60.02 | 2.58 | 0.61 | 0.23 | 0.51 | 0.16 |
| TG039639 | 62.12 | 2.45 | 1.21 | 0.28 | 0.76 | 0.11 | 60.32 | 16.04 | 0.77 | 0.34 | 0.78 | 0.32 |
| TG039638 | 68.26 | 1.47 | 1.31 | 0.25 | 0.81 | 0.22 | 72.76 | 2.29 | 0.99 | 0.15 | 0.99 | 0.02 |
| TG039637 | 47.56 | 2.97 | 0.93 | 0.20 | 0.65 | 0.16 | 59.96 | 3.30 | 0.67 | 0.05 | 0.89 | 0.38 |
| TG039636 | 26.95 | 1.92 | 0.45 | 0.23 | 0.23 | 0.05 | 50.16 | 5.17 | 0.79 | 0.09 | 0.61 | 0.08 |

### Example 54—Editing at the VEGFA Locus in Huh7 Cells Using tgRNAs with dNTPs in the DRS Region and with Modifications in the Spacer Sequence

Editing efficiency was assessed at the VEGFA locus in huh7 cells when tgRNAs with a DRS comprising dNTPs, and a spacer comprising modified nucleotides, were used along with a mRNA encoding a nCas9-polymerase fusion protein.

Huh7 cells were prepared as described in Example 1, and plated in 96-well plates (Falcon, 353072) at a density of 15,000 cells/well. Lipofectamine MessengerMAX (ThermoFisher Scientific, LMRNA015) lipoplexes were prepared according to manufacturer's instructions, with 10 ng/well of tgRNA and 100 ng/well of mRNA encoding a SpyCas9 nickase fused to one of the following moieties: a Kappa variant (nCas9-PolKv2) (SEQ ID NO: 1371), a wild-type T5 polymerase (nCas9-T5(WT)) (SEQ ID NO: 1301), or a T5 polymerase variant (nCas9-T5(D164A, E166A)) (SEQ ID NO: 1304). The tgRNAs comprised a spacer targeting the VEGFA locus, a scaffold, a template sequence encoding a GAGG-to-CACT edit, and a DRS sequence. The tgRNAs differed in the dNTP/rNTP composition of the DRS region, and in the modifications to nucleotides of the spacer region, as shown in Table 117.

TABLE 117
Exemplary tgRNAs with various dNTP/rNTP compositions in the DRS region and
various modifications in the spacer region.

| | | dNTP | | |
|---|---|---|---|---|
| | | positions | | |
| | Spacer | in the DRS | | |
| Guide ID | modifications | region | Spacer sequence | DRS sequence |
| TG039607 | none | D1 | mG*mA*mU*GUCUG | dTCUGGmCmCmUmGm |
| (SEQ ID | | | CAGGCCAGAUGA | CmA*mG*mU (SEQ ID |
| NO: 546) | | | (SEQ ID NO: 966) | NO: 952) |
| TG039613 | none | D1, D6-D12 | mG*mA*mU*GUCUG | dTCUGGdCdCdTdGdCd |
| (SEQ ID | | | CAGGCCAGAUGA | A*dG*mU (SEQ ID NO: |
| NO: 552) | | | (SEQ ID NO: 966) | 942) |
| TG039636 | none | D1-D12 | mG*mA*mU*GUCUG | dTdCdTdGdGdCdCdTdG |
| (SEQ ID | | | CAGGCCAGAUGA | dCdA*dG*mU (SEQ ID |
| NO: 575) | | | (SEQ ID NO: 966) | NO: 965) |
| TG039635 | none | D1-D13 | mG*mA*mU*GUCUG | dTdCdTdGdGdCdCdTdG |
| (SEQ ID | | | CAGGCCAGAUGA | dCdA*dG*dT (SEQ ID |
| NO: 574) | | | (SEQ ID NO: 966) | NO: 964) |
| TG039638 | 2′Ome at S9- | D1-D12 | mG*mA*mU*GUCUG | dTdCdTdGdGdCdCdTdG |
| (SEQ ID | S10, S13-S14, | | mCmAGGmCmCAGm | dCdA*dG*mU (SEQ ID |
| NO: 584) | S17, S20 | | AUGmA (SEQ ID NO: | NO: 965) |
| | | | 967) | |
| TG039642 | 2′Ome at S9- | D1-D12 | mG*mA*mU*GUCUG | dTdCdTdGdGdCdCdTdG |
| (SEQ ID | S10, S13-S14, | | mCmAdGdGmCmCA | dCdA*dG*mU (SEQ ID |
| NO: 580) | S17, S20, and | | GmAdTGmA (SEQ ID | NO: 965) |
| | dNTPs at S11- | | NO: 968) | |
| | S12, S18 | | | |

Note:
*denotes that the nucleotide is linked to the next nucleotide with a phosphorothioate (PS bond), a lower case “m” denotes that the nucleotide is modified with 2′-O-Me, and a lowercase “d” denotes that the nucleotide is a DNA nucleotide.

The cells were lysed 72 hours post-treatment for NGS analysis as described in Example 1. Samples were run in triplicates, unless otherwise indicated.

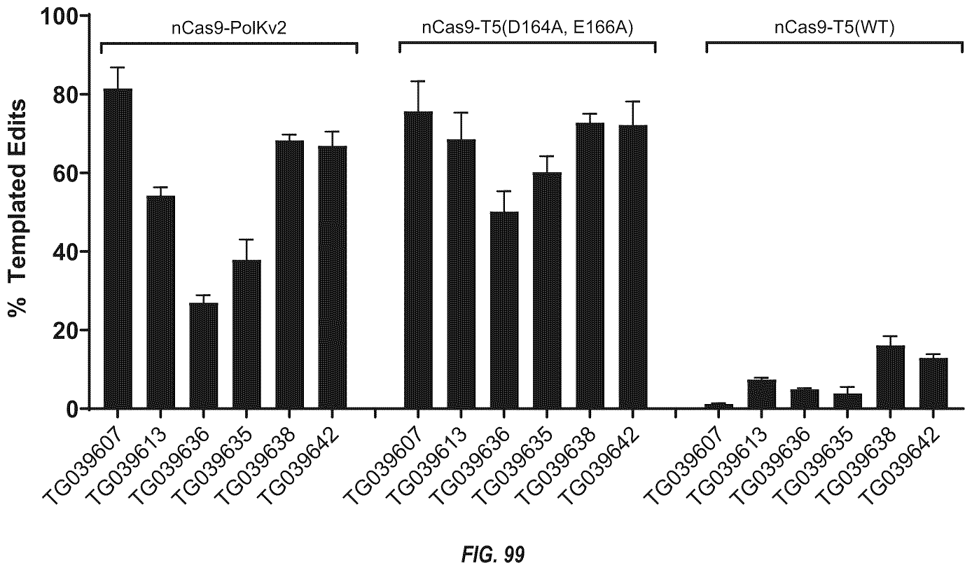

Tables 118 and 119 and FIG. 99 show the editing results at the VEGFA locus in huh7 cells treated with tgRNAs comprising different dNTP/rNTP compositions in the DRS region, and various modifications of the nucleotides in the spacer region.

TABLE 118
Editing at the VEGFA locus in huh7 cells treated with a mRNA encoding a nCas9-polymerase
fusion protein selected from nCas9-PolKv2, nCas9-T5(WT), or nCas9-T5(D164A, E166A), and
with tgRNAs encoding a GAGG-to-CACT edit, and comprising different dNTP/rNTP compositions
in the DRS region, and various modifications of the nucleotides in the spacer region.

| | | | TG039607 | TG039613 | TG039636 | TG039635 | TG039638 | TG039642 |
|---|---|---|---|---|---|---|---|---|
| nCas9-PolKv2 | % Templated | Mean | 81.29 | 54.20 | 26.95 | 37.85 | 68.26 | 66.84 |
| | Edits | SD | 6.72 | 2.14 | 1.92 | 5.20 | 1.47 | 3.67 |
| | % Templated + | Mean | 0.78 | 0.90 | 0.45 | 0.56 | 1.31 | 1.18 |
| | Byproduct | SD | 0.10 | 0.17 | 0.23 | 0.22 | 0.25 | 0.22 |
| | Edits | | | | | | | |
| | % Indels | Mean | 1.04 | 0.76 | 0.23 | 0.39 | 0.81 | 0.59 |
| | | SD | 0.07 | 0.22 | 0.05 | 0.08 | 0.22 | 0.09 |
| nCas9- | % Templated | Mean | 62.52 | 68.55 | 50.16 | 60.16 | 72.76 | 72.17 |
| T5(D164A, | Edits | SD | 9.46 | 6.78 | 5.17 | 4.08 | 2.29 | 5.96 |
| E166A) | % Templated + | Mean | 0.58 | 0.66 | 0.79 | 0.65 | 0.99 | 1.01 |
| | Byproduct | SD | 0.12 | 0.31 | 0.09 | 0.10 | 0.15 | 0.14 |
| | Edits | | | | | | | |
| | % Indels | Mean | 0.80 | 0.87 | 0.61 | 1.15 | 0.99 | 0.74 |
| | | SD | 0.37 | 0.30 | 0.08 | 1.12 | 0.02 | 0.22 |
| nCas9-T5(WT) | % Templated | Mean | 0.29 | 7.40 | 4.91 | 3.91 | 16.10 | 12.91 |
| | Edits | SD | 0.04 | 0.46 | 0.28 | 1.64 | 2.36 | 0.95 |
| | % Templated + | Mean | 0.20 | 0.26 | 0.32 | 0.80 | 0.51 | 0.35 |
| | Byproduct | SD | 0.04 | 0.13 | 0.11 | 0.43 | 0.09 | 0.19 |
| | Edits | | | | | | | |
| | % Indels | Mean | 6.51 | 5.13 | 5.49 | 7.68 | 5.33 | 3.94 |
| | | SD | 1.46 | 1.76 | 0.70 | 2.36 | 0.38 | 0.10 |

TABLE 119
Editing at the VEGFA locus in huh7 cells treated with a mRNA encoding a nCas9-polymerase
fusion protein selected from nCas9-PolKv2, nCas9-T5(WT), or nCas9-T5(D164A, E166A), and
with tgRNAs encoding a GAGG-to-CACT edit, and comprising different dNTP/rNTP compositions
in the DRS region, and various modifications of the nucleotides in the spacer region.

| | | | TG039607 | TG039613 | TG039636 | TG039635 | TG039638 | TG039642 |
|---|---|---|---|---|---|---|---|---|
| nCas9- | % | Mean | 81.29 | 54.20 | 26.95 | 37.85 | 68.26 | 66.84 |
| PolKv2 | Templated | SD | 6.72 | 2.14 | 1.92 | 5.20 | 1.47 | 3.67 |
| | Edits | | | | | | | |
| | % | Mean | 0.78 | 0.90 | 0.45 | 0.56 | 1.31 | 1.18 |
| | Templated + | SD | 0.10 | 0.17 | 0.23 | 0.22 | 0.25 | 0.22 |
| | Byproduct | | | | | | | |
| | Edits | | | | | | | |
| | % Indels | Mean | 1.04 | 0.76 | 0.23 | 0.39 | 0.81 | 0.59 |
| | | SD | 0.07 | 0.22 | 0.05 | 0.08 | 0.22 | 0.09 |
| nCas9- | % | Mean | 62.52 | 68.55 | 50.16 | 60.16 | 72.76 | 72.17 |
| T5(D164A, | Templated | SD | 9.46 | 6.78 | 5.17 | 4.08 | 2.29 | 5.96 |
| E166A) | Edits | | | | | | | |
| | % | Mean | 0.58 | 0.66 | 0.79 | 0.65 | 0.99 | 1.01 |
| | Templated + | SD | 0.12 | 0.31 | 0.09 | 0.10 | 0.15 | 0.14 |
| | Byproduct | | | | | | | |
| | Edits | | | | | | | |
| | % Indels | Mean | 0.80 | 0.87 | 0.61 | 1.15 | 0.99 | 0.74 |
| | | SD | 0.37 | 0.30 | 0.08 | 1.12 | 0.02 | 0.22 |
| nCas9- | % | Mean | 0.29 | 7.40 | 4.91 | 3.91 | 16.10 | 12.91 |
| T5(WT) | Templated | SD | 0.04 | 0.46 | 0.28 | 1.64 | 2.36 | 0.95 |
| | Edits | | | | | | | |
| | % | Mean | 0.20 | 0.26 | 0.32 | 0.80 | 0.51 | 0.35 |
| | Templated + | SD | 0.04 | 0.13 | 0.11 | 0.43 | 0.09 | 0.19 |
| | Byproduct | | | | | | | |
| | Edits | | | | | | | |
| | % Indels | Mean | 6.51 | 5.13 | 5.49 | 7.68 | 5.33 | 3.94 |
| | | SD | 1.46 | 1.76 | 0.70 | 2.36 | 0.38 | 0.10 |

### Example 55—Editing at the VEGFA Locus in PHH Using tgRNAs with Modified Nucleotides at The Template:DRS Junction

In this Example, templated editing efficiency was assessed in primary human hepatocytes with tgRNAs having various DNA/RNA compositions at the junction between the template sequence and the DNA Polymerase Recruiting Sequence (DRS).

A dose response experiment was conducted in Primary human hepatocytes (PHH, Gibco, Lot 8381) prepared as described in Example 1, and plated in 96-well plates (Falcon, 353072) at a density of 35,000 cells/well, using a tgRNA selected from the tgRNAs listed in Table 111, and a mRNA encoding one of nCas9-PolKv2 (SEQ ID NO: 1371) or nCas9-T5(D164A, E166A) (SEQ ID NO: 1304), in the presence of Vpx (SEQ ID NO: 1462) and FEN1 (SEQ ID NO: 1465).

LNPs were prepared as described in Example 1, with a molar ratio of 50% Lipid A, 38% cholesterol, 9% DSPC, and 3% PEG2k-DMG, and formulated with a lipid amine to RNA phosphate (N:P) molar ratio of about 6. LNPs comprised a single mRNA species encoding one of nCas9-PolKv2, nCas9-T5 (D164A, E166A), Vpx or FEN1. The tgRNAs were delivered using pre-mixed lipid formulations, prepared as described in Example 1, with a molar ratio of 50% Lipid A, 38% cholesterol, 9% DSPC, and 3% PEG2k-DMG.

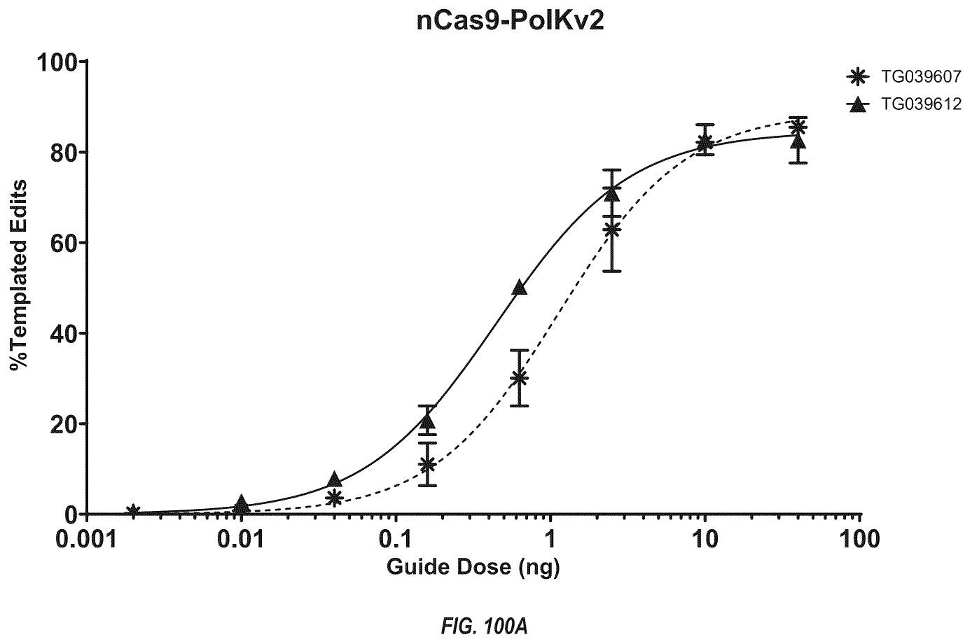

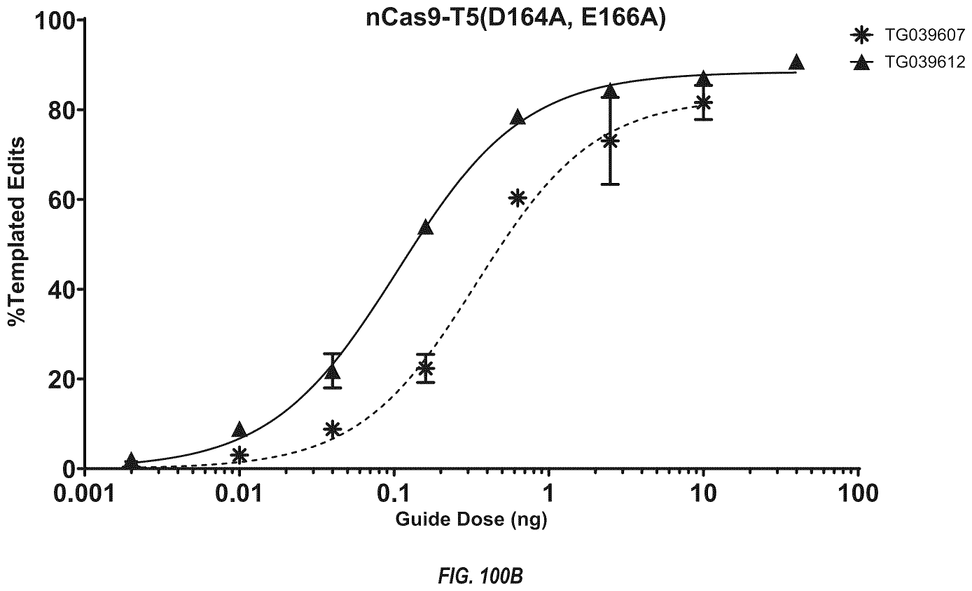

Cells were incubated at 37° C., 5% CO₂ for 24 hours prior to treatment with LNPs and the pre-mixed lipid formulations. LNPs and lipid formulations were incubated in media containing 10% fetal bovine serum (FBS) at 37° C. for 10 minutes. Post-incubation, cells were treated with 10 ng of mRNA encoding one of nCas9-PolKv2 or nCas9-T5(D164A, E166A), 5 ng of mRNA encoding VPX, and 25 ng of mRNA encoding FEN1, along with a tgRNA in an 8-point 4-fold dilution series starting at 40 ng of tgRNA. The cells were lysed 72 hours post-treatment for NGS analysis as described in Example 1. Samples were run in triplicates, unless otherwise indicated. Table 120 and FIGS. 100A and B show the editing results at the VEGFA locus in PHH treated with tgRNAs comprising different dNTP/rNTP compositions at the template:DRS junction.

TABLE 120
Editing at the VEGFA locus in PHH treated with tgRNAs comprising
different dNTP/rNTP compositions at the template: DRS junction

| | | | tgRNA | | | | | | | | |
|---|---|---|---|---|---|---|---|---|---|---|---|
| mRNA ID | Guide ID | Edit Type | (ng) | 40 | 10 | 2.5 | 0.63 | 0.16 | 0.04 | 0.01 | 0.002 |
| nCas9- | TG039607 | % Templated | Mean | 85.51* | 82.19* | 62.87** | 30.06 | 11.02** | 3.62 | 0.78 | 0.16 |
| PolKv2 | | Edits | SD | N/A | N/A | 9.20** | 6.14 | 4.71** | 0.12 | 0.75 | 0.13 |
| | | % Templated + | Mean | 2.14 | 1.87* | 1.39** | 0.77 | 0.39** | 0.03 | 0.01 | 0.00 |
| | | Byproduct | SD | N/A | N/A | 0.20** | 0.11 | 0.24** | 0.02 | 0.01 | 0.00 |
| | | Edits | | | | | | | | | |
| | | % Indels | Mean | 0.64* | 0.58* | 0.57** | 0.20 | 0.17** | 0.01 | 0.03 | 0.00 |
| | | | SD | N/A | N/A | 0.23** | 0.07 | 0.02** | 0.01 | 0.05 | 0.00 |
| | TG039612 | % Templated | Mean | 82.62 | 82.77 | 70.94 | 50.34 | 20.76 | 7.91 | 2.75 | 0.61 |
| | | Edits | SD | 5.03 | 3.32 | 5.14 | 1.22 | 3.18 | 0.87 | 0.6 | 0.3 |
| | | % Templated + | Mean | 2.30 | 1.83 | 1.99 | 1.10 | 0.68 | 0.43 | 0.07 | 0.00 |
| | | Byproduct | SD | 0.60 | 0.47 | 0.42 | 0.10 | 0.32 | 0.14 | 0.07 | 0.00 |
| | | Edits | | | | | | | | | |
| | | % Indels | Mean | 1.16 | 1.06 | 0.89 | 0.50 | 0.20 | 0.20 | 0.02 | 0.01 |
| | | | SD | 0.35 | 0.62 | 0.34 | 0.06 | 0.08 | 0.28 | 0.03 | 0.00 |
| nCas9- | TG039607 | % Templated | Mean | N/A | 81.62** | 73.06** | 60.34* | 22.34 | 8.80 | 3.02** | 0.36 |
| T5(D164A, | | Edits | SD | N/A | 3.83** | 9.68** | N/A | 3.14 | 1.10 | 0.95** | 0.50 |
| E166A) | | % Templated + | Mean | N/A | 0.85** | 0.75** | 1.00* | 0.43 | 0.22 | 0.03** | 0.00 |
| | | Byproduct | SD | N/A | 0.72** | 0.42** | N/A | 0.39 | 0.23 | 0.04** | 0.00 |
| | | Edits | | | | | | | | | |
| | | % Indels | Mean | N/A | 0.81** | 0.73** | 0.54* | 0.25 | 0.23 | 0.04** | 0.00 |
| | | | SD | N/A | 0.22** | 0.69** | N/A | 0.07 | 0.31 | 0.05** | 0.00 |
| | TG039612 | % Templated | Mean | 90.78* | 87.12 | 84.36 | 78.55** | 54.01** | 21.81 | 8.9 | 2.04** |
| | | Edits | SD | N/A | 1.36 | 1.17 | 0.63** | 0.65** | 3.8 | 1.54 | 0.43** |
| | | % Templated + | Mean | 1.02* | 1.02 | 0.68 | 0.95** | 0.58** | 0.27 | 0.07 | 0.00** |
| | | Byproduct | SD | N/A | 0.41 | 0.27 | 0.47** | 0.27** | 0.15 | 0.11 | 0.00** |
| | | Edits | | | | | | | | | |
| | | % Indels | Mean | 1.08* | 0.70 | 0.58 | 0.71 | 0.45** | 0.24 | 0.17 | 0.01** |
| | | | SD | N/A | 0.49 | 0.29 | 0.11** | 0.41** | 0.17 | 0.16 | 0.00** |

*N = 1;
**N = 2

### Example 56-Editing at the VEGFA Locus in PHH Using tgRNAs with Modified Nucleotides at Various Positions in the DRS Region

A dose response experiment was conducted in Primary human hepatocytes (PHH, Gibco, Lot 8381) prepared as described in Example 1, and plated in 96-well plates (Falcon, 353072) at a density of 35,000 cells/well, using a tgRNA selected from the tgRNAs listed in Table 113, and a mRNA encoding one of nCas9-PolKv2 (SEQ ID NO: 1371) or nCas9-T5(D164A, E166A) (SEQ ID NO: 1304), in the presence of Vpx (SEQ ID NO: 1462) and FEN1 (SEQ ID NO: 1465).

LNPs were prepared as described in Example 1, with a molar ratio of 50% Lipid A, 38% cholesterol, 9% DSPC, and 3% PEG2k-DMG, and formulated with a lipid amine to RNA phosphate (N:P) molar ratio of about 6. LNPs comprised a single mRNA species encoding one of nCas9-PolKv2, nCas9-T5 (D164A, E166A), Vpx or FEN1. The tgRNAs were delivered using pre-mixed lipid formulations, prepared as described in Example 1, with a molar ratio of 50% Lipid A, 38% cholesterol, 9% DSPC, and 3% PEG2k-DMG.

Cells were incubated at 37° C., 5% CO₂ for 24 hours prior to treatment with LNPs and the pre-mixed lipid formulations. LNPs and lipid formulations were incubated in media containing 10% fetal bovine serum (FBS) at 37° C. for 10 minutes. Post-incubation, cells were treated with 10 ng of mRNA encoding one of nCas9-PolKv2 or nCas9-T5(D164A, E166A), 5 ng of mRNA encoding VPX, and 25 ng of mRNA encoding FEN1, along with a tgRNA in an 8-point 4-fold dilution series starting at 40 ng of tgRNA. The cells were lysed 72 hours post-treatment for NGS analysis as described in Example 1. Samples were run in triplicates, unless otherwise indicated.

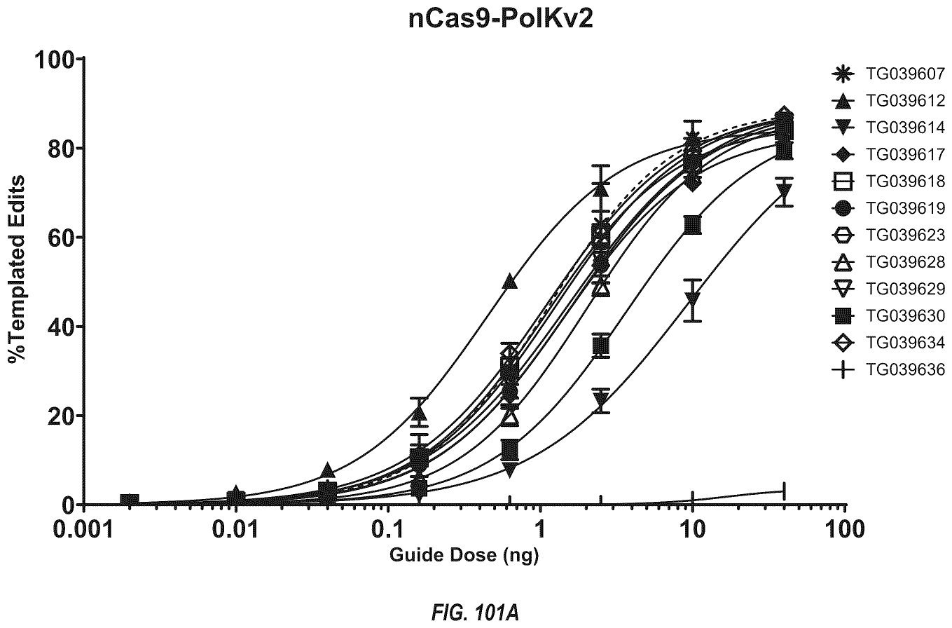

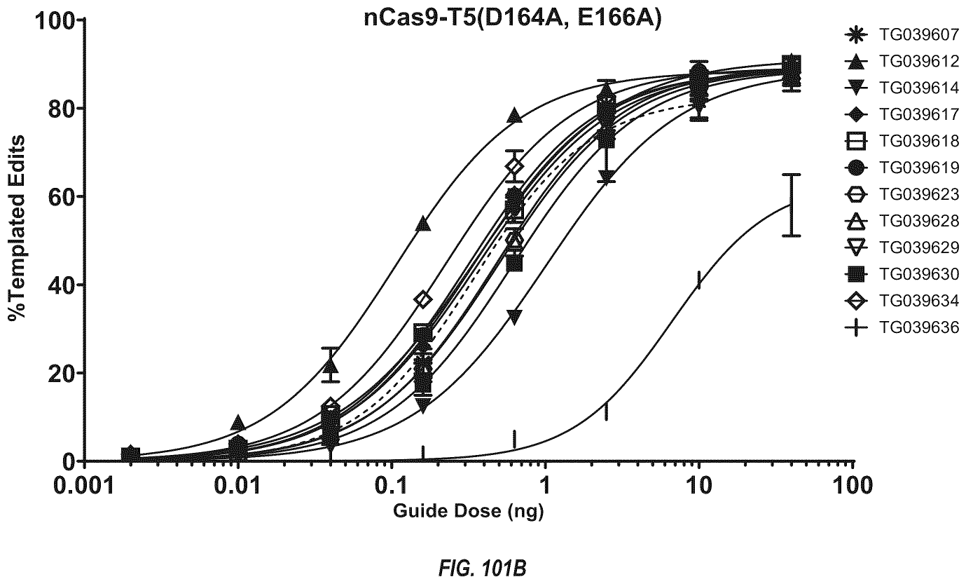

Table 121 and FIGS. 101A and B show the editing results at the VEGFA locus in PHH treated with tgRNAs comprising different dNTP/rNTP compositions in the DRS region.

TABLE 121
Editing at the VEGFA locus in PHH treated with tgRNAs comprising
different dNTP/rNTP compositions in the DRS region.

| | | | tgRNA | | | | | | | | |
|---|---|---|---|---|---|---|---|---|---|---|---|
| mRNA ID | Guide ID | Edit Type | (ng) | 40 | 10 | 2.5 | 0.63 | 0.16 | 0.04 | 0.01 | 0.002 |
| nCas9- | TG039607 | % Templated | Mean | 85.51* | 82.19* | 62.87** | 30.06 | 11.02** | 3.62 | 0.78 | 0.16 |
| PolKv2 | | Edits | SD | N/A | N/A | 9.20** | 6.14 | 4.71** | 0.12 | 0.75 | 0.13 |
| | | % Templated + | Mean | 2.14* | 1.87* | 1.39** | 0.77 | 0.39** | 0.03 | 0.01 | 0.00 |
| | | Byproduct | SD | N/A | N/A | 0.20** | 0.11 | 0.24** | 0.02 | 0.01 | 0.00 |
| | | Edits | | | | | | | | | |
| | | % Indels | Mean | 0.64* | 0.58* | 0.57** | 0.20 | 0.17** | 0.01 | 0.03 | 0.00 |
| | | | SD | N/A | N/A | 0.23** | 0.07 | 0.02** | 0.01 | 0.05 | 0.00 |
| | TG039612 | % Templated | Mean | 82.62 | 82.77 | 70.94 | 50.34 | 20.76 | 7.91 | 2.75 | 0.61 |
| | | Edits | SD | 5.03 | 3.32 | 5.14 | 1.22 | 3.18 | 0.87 | 0.6 | 0.3 |
| | | % Templated + | Mean | 2.30 | 1.83 | 1.99 | 1.10 | 0.68 | 0.43 | 0.07 | 0.00 |
| | | Byproduct | SD | 0.60 | 0.47 | 0.42 | 0.10 | 0.32 | 0.14 | 0.07 | 0.00 |
| | | Edits | | | | | | | | | |
| | | % Indels | Mean | 1.16 | 1.06 | 0.89 | 0.50 | 0.20 | 0.20 | 0.02 | 0.01 |
| | | | SD | 0.35 | 0.62 | 0.34 | 0.06 | 0.08 | 0.28 | 0.03 | 0.00 |
| | TG039614 | % Templated | Mean | 70.13 | 45.79 | 23.28 | 7.70 | 2.11 | 0.45 | 0.41 | 0.04 |
| | | Edits | SD | 3.13 | 4.64 | 2.63 | 1.27 | 0.49 | 0.06 | 0.19 | 0.03 |
| | | % Templated + | Mean | 1.64 | 1.14 | 0.66 | 0.10 | 0.06 | 0.04 | 0.00 | 0.00 |
| | | Byproduct | SD | 0.19 | 0.46 | 0.24 | 0.06 | 0.07 | 0.05 | 0.00 | 0.00 |
| | | Edits | | | | | | | | | |
| | | % Indels | Mean | 0.82 | 0.67 | 0.37 | 0.19 | 0.04 | 0.00 | 0.01 | 0.00 |
| | | | SD | 0.32 | 0.32 | 0.05 | 0.09 | 0.03 | 0.00 | 0.00 | 0.00 |
| | TG039617 | % Templated | Mean | 82.28** | 71.90 | 54.84 | 24.30** | 8.43 | 3.27 | 0.62 | 0.05** |
| | | Edits | SD | 1.37** | 0.48 | 5.12 | 2.68** | 1.19 | 0.33 | 0.14 | 0.03** |
| | | % Templated + | Mean | 1.77** | 1.93 | 1.33 | 0.85** | 0.25 | 0.04 | 0.00 | 0.02** |
| | | Byproduct | SD | 0.23** | 0.17 | 0.39 | 0.16** | 0.26 | 0.06 | 0.00 | 0.03** |
| | | Edits | | | | | | | | | |
| | | % Indels | Mean | 0.59** | 0.84 | 0.46 | 0.21** | 0.10 | 0.03 | 0.00 | 0.03** |
| | | | SD | 0.31** | 0.14 | 0.11 | 0.14** | 0.10 | 0.05 | 0.00 | 0.03** |
| | TG039618 | % Templated | Mean | 83.97 | 76.47 | 60.39 | 31.03 | 10.53 | 3.05 | 0.69 | 0.25 |
| | | Edits | SD | 2.72 | 2.99 | 2.06 | 2.02 | 1.72 | 0.27 | 0.05 | 0.02 |
| | | % Templated + | Mean | 2.21 | 1.76 | 1.48 | 0.65 | 0.23 | 0.06 | 0.01 | 0.00 |
| | | Byproduct | SD | 0.26 | 0.23 | 0.29 | 0.19 | 0.12 | 0.07 | 0.01 | 0.00 |
| | | Edits | | | | | | | | | |
| | | % Indels | Mean | 1.06 | 0.67 | 0.53 | 0.25 | 0.09 | 0.04 | 0.04 | 0.00 |
| | | | SD | 0.22 | 0.30 | 0.24 | 0.29 | 0.03 | 0.06 | 0.05 | 0.00 |
| | TG039619 | % Templated | Mean | 84.77 | 76.95** | 53.76 | 25.43 | 8.96 | 2.62 | 0.61 | 0.24 |
| | | Edits | SD | 2.10 | 1.40** | 3.94 | 2.98 | 0.52 | 1.09 | 0.30 | 0.16 |
| | | % Templated + | Mean | 1.38 | 1.57** | 0.74 | 0.83 | 0.23 | 0.11 | 0.00 | 0.00 |
| | | Byproduct | SD | 0.36 | 0.13** | 0.34 | 0.32 | 0.11 | 0.13 | 0.00 | 0.00 |
| | | Edits | | | | | | | | | |
| | | % Indels | Mean | 0.62 | 1.00** | 0.33 | 0.32 | 0.13 | 0.08 | 0.01 | 0.02 |
| | | | SD | 0.27 | 0.31** | 0.28 | 0.18 | 0.05 | 0.08 | 0.01 | 0.03 |
| | TG039623 | % Templated | Mean | 85.91 | 79.35 | 58.94 | 29.71 | 10.37 | 3.18 | 0.97 | 0.23 |
| | | Edits | SD | 0.49 | 1.21 | 1.29 | 1.29 | 1.41 | 0.29 | 0.08 | 0.06 |
| | | % Templated + | Mean | 1.57 | 1.79 | 1.47 | 0.80 | 0.33 | 0.03 | 0.03 | 0.00 |
| | | Byproduct | SD | 0.14 | 0.29 | 0.17 | 0.13 | 0.12 | 0.02 | 0.03 | 0.00 |
| | | Edits | | | | | | | | | |
| | | % Indels | Mean | 0.92 | 0.81 | 0.41 | 0.23 | 0.06 | 0.02 | 0.07 | 0.00 |
| | | | SD | 0.34 | 0.13 | 0.13 | 0.14 | 0.05 | 0.03 | 0.03 | 0.00 |
| | TG039628 | % Templated | Mean | 84.41 | 73.88 | 49.10 | 20.01 | 5.83 | 1.62 | 0.87 | 0.20 |
| | | Edits | SD | 0.44 | 1.16 | 2.24 | 2.22 | 0.53 | 0.88 | 0.22 | 0.11 |
| | | % Templated + | Mean | 1.86 | 1.74 | 1.34 | 0.61 | 0.14 | 0.02 | 0.01 | 0.00 |
| | | Byproduct | SD | 0.08 | 0.23 | 0.43 | 0.17 | 0.08 | 0.02 | 0.00 | 0.00 |
| | | Edits | | | | | | | | | |
| | | % Indels | Mean | 0.79 | 0.63 | 0.60 | 0.15 | 0.08 | 0.01 | 0.00 | 0.01 |
| | | | SD | 0.22 | 0.27 | 0.25 | 0.11 | 0.06 | 0.01 | 0.00 | 0.00 |
| | TG039629 | % Templated | Mean | 86.04 | 76.52 | 54.84 | 27.72 | 9.69 | 2.50 | 0.95 | 0.25 |
| | | Edits | SD | 1.37 | 1.32 | 1.07 | 0.80 | 0.26 | 0.72 | 0.09 | 0.15 |
| | | % Templated + | Mean | 1.78 | 1.49 | 1.61 | 0.71 | 0.25 | 0.13 | 0.00 | 0.00 |
| | | Byproduct | SD | 0.32 | 0.67 | 0.35 | 0.13 | 0.08 | 0.04 | 0.01 | 0.00 |
| | | Edits | | | | | | | | | |
| | | % Indels | Mean | 0.75 | 0.67 | 0.63 | 0.15 | 0.08 | 0.06 | 0.00 | 0.00 |
| | | | SD | 0.11 | 0.09 | 0.09 | 0.15 | 0.04 | 0.09 | 0.00 | 0.00 |
| | TG039630 | % Templated | Mean | 79.29 | 62.77 | 35.68 | 12.32 | 3.66 | 1.14 | 0.17 | 0.09 |
| | | Edits | SD | 1.07 | 1.90 | 2.62 | 2.18 | 0.12 | 0.30 | 0.13 | 0.13 |
| | | % Templated + | Mean | 1.53 | 2.10 | 1.19 | 0.29 | 0.14 | 0.13 | 0.00 | 0.02 |
| | | Byproduct | SD | 0.45 | 0.19 | 0.29 | 0.38 | 0.11 | 0.20 | 0.00 | 0.03 |
| | | Edits | | | | | | | | | |
| | | % Indels | Mean | 0.70 | 0.84 | 0.60 | 0.17 | 0.03 | 0.01 | 0.00 | 0.01 |
| | | | SD | 0.19 | 0.19 | 0.07 | 0.18 | 0.02 | 0.01 | 0.00 | 0.01 |
| | TG039634 | % Templated | Mean | 87.41 | 78.54 | 61.71 | 33.97 | 11.19 | 3.13 | 0.79 | 0.34 |
| | | Edits | SD | 1.47 | 2.27 | 1.50 | 0.36 | 2.28 | 0.91 | 0.31 | 0.43 |
| | | % Templated + | Mean | 1.83 | 2.14 | 1.68 | 0.93 | 0.42 | 0.07 | 0.02 | 0.03 |
| | | Byproduct | SD | 0.31 | 0.29 | 0.16 | 0.14 | 0.18 | 0.03 | 0.01 | 0.04 |
| | | Edits | | | | | | | | | |
| | | % Indels | Mean | 0.72 | 0.74 | 0.73 | 0.39 | 0.19 | 0.01 | 0.00 | 0.01 |
| | | | SD | 0.19 | 0.24 | 0.07 | 0.17 | 0.06 | 0.00 | 0.00 | 0.00 |
| | TG039636 | % Templated | Mean | 3.02 | 1.01** | 0.10 | 0.02 | 0.01 | 0.00 | 0.00 | 0.09 |
| | | Edits | SD | 0.18 | 0.48** | 0.12 | 0.03 | 0.01 | 0.00 | 0.00 | 0.15 |
| | | % Templated + | Mean | 0.09 | 0.01** | 0.01 | 0.00 | 0.00 | 0.00 | 0.00 | 0.00 |
| | | Byproduct | SD | 0.07 | 0.01** | 0.01 | 0.00 | 0.00 | 0.00 | 0.00 | 0.00 |
| | | Edits | | | | | | | | | |
| | | % Indels | Mean | 0.07 | 0.00** | 0.01 | 0.01 | 0.00 | 0.00 | 0.01 | 0.01 |
| | | | SD | 0.06 | 0.00** | 0.01 | 0.01 | 0.00 | 0.00 | 0.01 | 0.00 |
| nCas9- | TG039607 | % Templated | Mean | N/A | 81.62** | 73.06** | 60.34* | 22.34 | 8.8 | 3.02** | 0.36 |
| T5(D164A, | | Edits | SD | N/A | 3.83** | 9.68** | N/A | 3.14 | 1.1 | 0.95** | 0.5 |
| E166A) | | % Templated + | Mean | N/A | 0.85** | 0.75** | 1.00* | 0.43 | 0.22 | 0.03** | 0.00 |
| | | Byproduct | SD | N/A | 0.72** | 0.42** | N/A | 0.39 | 0.23 | 0.04** | 0.00 |
| | | Edits | | | | | | | | | |
| | | % Indels | Mean | N/A | 0.81** | 0.73** | 0.54* | 0.25 | 0.23 | 0.04** | 0.00 |
| | | | SD | N/A | 0.22** | 0.69** | N/A | 0.07 | 0.31 | 0.05** | 0.00 |
| | TG039612 | % Templated | Mean | 90.78* | 87.12 | 84.36 | 78.55** | 54.01** | 21.81 | 8.9 | 2.04** |
| | | Edits | SD | N/A | 1.36 | 1.17 | 0.63** | 0.65** | 3.8 | 1.54 | 0.43** |
| | | % Templated + | Mean | 1.02* | 1.02 | 0.68 | 0.95** | 0.58** | 0.27 | 0.07 | 0.00** |
| | | Byproduct | SD | N/A | 0.41 | 0.27 | 0.47** | 0.27** | 0.15 | 0.11 | 0.00** |
| | | Edits | | | | | | | | | |
| | | % Indels | Mean | 1.08* | 0.70 | 0.58 | 0.71 | 0.45** | 0.24 | 0.17 | 0.01** |
| | | | SD | N/A | 0.49 | 0.29 | 0.11** | 0.41** | 0.17 | 0.16 | 0.00** |
| | TG039614 | % Templated | Mean | 86.97 | 80.18 | 64.12** | 32.49* | 12.42 | 3.30 | 0.77 | 0.32** |
| | | Edits | SD | 0.54 | 2.93 | 0.38** | N/A | 0.98 | 0.22 | 0.17 | 0.17** |
| | | % Templated + | Mean | 1.01 | 0.77 | 0.89** | 0.47* | 0.08 | 0.06 | 0.00 | 0.00** |
| | | Byproduct | SD | 0.11 | 0.17 | 0.08** | N/A | 0.04 | 0.07 | 0.01 | 0.00** |
| | | Edits | | | | | | | | | |
| | | % Indels | Mean | 0.98 | 0.57 | 0.78** | 0.59* | 0.09 | 0.06 | 0.01 | 0.00** |
| | | | SD | 0.42 | 0.29 | 0.03** | N/A | 0.07 | 0.06 | 0.01 | 0.00** |
| | TG039617 | % Templated | Mean | 88.59 | 85.33 | 77.96 | 57.23 | 26.03 | 9.85 | 2.46 | 1.39** |
| | | Edits | SD | 0.75 | 2.49 | 0.28 | 1.56 | 3.85 | 0.70 | 0.57 | 0.76** |
| | | % Templated + | Mean | 1.12 | 1.05 | 1.23 | 0.75 | 0.76 | 0.05 | 0.05 | 0.01** |
| | | Byproduct | SD | 0.25 | 0.18 | 0.83 | 0.21 | 0.32 | 0.05 | 0.01 | 0.01** |
| | | Edits | | | | | | | | | |
| | | % Indels | Mean | 1.10 | 0.98 | 0.80 | 0.92 | 0.52 | 0.03 | 0.09 | 0.03** |
| | | | SD | 0.29 | 0.38 | 0.92 | 0.30 | 0.34 | 0.03 | 0.10 | 0.04** |
| | TG039618 | % Templated | Mean | 89.90** | 86.69** | 80.82 | 56.96 | 29.12 | 10.57 | 2.75 | 0.96 |
| | | Edits | SD | 1.44** | 1.45** | 0.61 | 2.83 | 1.74 | 1.37 | 0.75 | 0.31 |
| | | % Templated + | Mean | 1.05** | 0.89** | 0.99 | 0.58 | 0.34 | 0.12 | 0.01 | 0.01 |
| | | Byproduct | SD | 0.26** | 0.23** | 0.22 | 0.22 | 0.06 | 0.09 | 0.00 | 0.01 |
| | | Edits | | | | | | | | | |
| | | % Indels | Mean | 1.02** | 0.77** | 0.83 | 0.60 | 0.19 | 0.09 | 0.01 | 0.01 |
| | | | SD | 0.08** | 0.92** | 0.22 | 0.36 | 0.07 | 0.09 | 0.01 | 0.02 |
| | TG039619 | % Templated | Mean | 86.74 | 88.37** | 79.69 | 60.25 | 28.56 | 9.55 | 4.00 | 1.19 |
| | | Edits | SD | 2.82 | 0.82** | 0.83 | 0.20 | 0.73 | 0.18 | 0.83 | 0.88 |
| | | % Templated + | Mean | 0.88 | 0.81** | 0.70 | 0.52 | 0.27 | 0.12 | 0.01 | 0.04 |
| | | Byproduct | SD | 0.10 | 0.05** | 0.28 | 0.11 | 0.06 | 0.17 | 0.00 | 0.07 |
| | | Edits | | | | | | | | | |
| | | % Indels | Mean | 0.69 | 0.94** | 0.71 | 0.46 | 0.27 | 0.13 | 0.03 | 0.07 |
| | | | SD | 0.32 | 0.24** | 0.57 | 0.08 | 0.07 | 0.11 | 0.04 | 0.11 |
| | TG039623 | % Templated | Mean | 86.73** | 86.74 | 77.87 | 50.09 | 20.94 | 8.21** | 3.15 | 0.99 |
| | | Edits | SD | 1.62** | 0.72 | 2.92 | 0.98 | 2.06 | 0.99** | 0.09 | 0.45 |
| | | % Templated + | Mean | 0.75** | 0.96 | 0.78 | 0.58 | 0.26 | 0.12** | 0.05 | 0.01 |
| | | Byproduct | SD | 0.41** | 0.36 | 0.02 | 0.23 | 0.15 | 0.07** | 0.05 | 0.01 |
| | | Edits | | | | | | | | | |
| | | % Indels | Mean | 0.94** | 0.99 | 0.51 | 0.61 | 0.18 | 0.17** | 0.06 | 0.00 |
| | | | SD | 0.23** | 0.32 | 0.24 | 0.29 | 0.09 | 0.21** | 0.10 | 0.01 |
| | TG039628 | % Templated | Mean | 86.95** | 86.08 | 75.65 | 49.61 | 21.21 | 6.57 | 1.95 | 0.76 |
| | | Edits | SD | 0.24** | 1.15 | 1.47 | 3.10 | 3.16 | 0.23 | 0.59 | 0.25 |
| | | % Templated + | Mean | 0.82** | 0.80 | 0.45 | 0.38 | 0.29 | 0.07 | 0.04 | 0.00 |
| | | Byproduct | SD | 0.12** | 0.20 | 0.12 | 0.23 | 0.07 | 0.07 | 0.06 | 0.00 |
| | | Edits | | | | | | | | | |
| | | % Indels | Mean | 1.24** | 0.98 | 0.49 | 0.36 | 0.15 | 0.07 | 0.03 | 0.00 |
| | | | SD | 0.22** | 0.25 | 0.37 | 0.29 | 0.09 | 0.10 | 0.05 | 0.00 |
| | TG039629 | % Templated | Mean | 87.96 | 86.62** | 79.19** | 58.57** | 27.49 | 9.38** | 2.58 | 0.70 |
| | | Edits | SD | 2.32 | 0.00** | 1.66** | 0.03** | 1.74 | 0.30** | 0.98 | 0.69 |
| | | % Templated + | Mean | 1.04 | 1.00** | 0.51** | 0.73** | 0.19 | 0.06** | 0.00 | 0.01 |
| | | Byproduct | SD | 0.74 | 0.17** | 0.38** | 0.10** | 0.19 | 0.09** | 0.00 | 0.01 |
| | | Edits | | | | | | | | | |
| | | % Indels | Mean | 0.78 | 0.66** | 0.73** | 0.41** | 0.13 | 0.04** | 0.00 | 0.01 |
| | | | SD | 0.47 | 0.62** | 0.37** | 0.17** | 0.20 | 0.04** | 0.00 | 0.01 |
| | TG039630 | % Templated | Mean | 87.28 | 85.14** | 72.77** | 44.80 | 17.42 | 5.63** | 2.48** | 1.01** |
| | | Edits | SD | 1.29 | 3.11** | 1.04** | 1.45 | 2.47 | 1.84** | 0.11** | 0.16** |
| | | % Templated + | Mean | 0.86 | 0.71** | 0.87** | 0.53 | 0.28 | 0.09** | 0.02** | 0.01** |
| | | Byproduct | SD | 0.10 | 0.18** | 0.36** | 0.38 | 0.10 | 0.10** | 0.02** | 0.01** |
| | | Edits | | | | | | | | | |
| | | % Indels | Mean | 0.83 | 0.78** | 0.70** | 0.51 | 0.14 | 0.03** | 0.01** | 0.01** |
| | | | SD | 0.19 | 0.22** | 0.52** | 0.16 | 0.09 | 0.02** | 0.01** | 0.02** |
| | TG039634 | % Templated | Mean | 88.13 | 88.35 | 82.58 | 66.84 | 36.69 | 12.48 | 3.91 | 1.58 |
| | | Edits | SD | 2.99 | 2.25 | 3.71 | 3.50 | 1.14 | 0.71 | 1.00 | 0.55 |
| | | % Templated + | Mean | 0.68 | 0.76 | 0.63 | 0.65 | 0.50 | 0.24 | 0.11 | 0.03 |
| | | Byproduct | SD | 0.16 | 0.20 | 0.31 | 0.09 | 0.13 | 0.07 | 0.13 | 0.03 |
| | | Edits | | | | | | | | | |
| | | % Indels | Mean | 0.65 | 0.97 | 0.90 | 0.62 | 0.36 | 0.25 | 0.09 | 0.02 |
| | | | SD | 0.09 | 0.18 | 0.17 | 0.17 | 0.18 | 0.17 | 0.15 | 0.02 |
| | TG039636 | % Templated | Mean | 58.01 | 41.04** | 11.26** | 4.99 | 1.45 | 0.36 | 0.04 | 0.02 |
| | | Edits | SD | 6.94 | 0.37** | 0.72** | 0.36 | 0.40 | 0.30 | 0.06 | 0.03 |
| | | % Templated + | Mean | 1.13 | 1.64** | 0.62 | 0.14 | 0.10 | 0.01 | 0.00 | 0.00 |
| | | Byproduct | SD | 0.72 | 0.47** | 0.02 | 0.04 | 0.10 | 0.01 | 0.00 | 0.00 |
| | | Edits | | | | | | | | | |
| | | % Indels | Mean | 1.51 | 2.28** | 0.75 | 0.28 | 0.05 | 0.01 | 0.00 | 0.01 |
| | | | SD | 0.83 | 0.26** | 0.20 | 0.09 | 0.05 | 0.01 | 0.00 | 0.00 |

*N = 1;
**N = 2

### Example 57—Editing at the VEGFA Locus in PHH Using tgRNAs with Modified Nucleotides at Various Positions in the Spacer Sequence

In this Example, templated editing efficiency in primary human hepatocytes (PHH) was assessed with tgRNAs having a spacer sequence with various DNA/RNA compositions or nucleotide modifications (e.g., 2′O-methyl(2′Ome) modifications).

A dose response experiment was conducted in Primary human hepatocytes (PHH, Gibco, Lot 8381) prepared as described in Example 1, and plated in 96-well plates (Falcon, 353072) at a density of 35,000 cells/well, using a tgRNA selected from the tgRNAs listed in Table 115, and a mRNA encoding one of nCas9-PolKv2 (SEQ ID NO: 1371) or nCas9-T5(D164A, E166A) (SEQ ID NO: 1304), in the presence of Vpx (SEQ ID NO: 1462) and FEN1 (SEQ ID NO: 1465).

LNPs were prepared as described in Example 1, with a molar ratio of 50% Lipid A, 38% cholesterol, 9% DSPC, and 3% PEG2k-DMG, and formulated with a lipid amine to RNA phosphate (N:P) molar ratio of about 6. LNPs comprised a single mRNA species encoding one of nCas9-PolKv2, nCas9-T5 (D164A, E166A), Vpx or FEN1. The tgRNAs were delivered using pre-mixed lipid formulations, prepared as described in Example 1, with a molar ratio of 50% Lipid A, 38% cholesterol, 9% DSPC, and 3% PEG2k-DMG.

Cells were incubated at 37° C., 5% CO₂ for 24 hours prior to treatment with LNPs and the pre-mixed lipid formulations. LNPs and lipid formulations were incubated in media containing 10% fetal bovine serum (FBS) at 37° C. for 10 minutes. Post-incubation, cells were treated with 10 ng of mRNA encoding one of nCas9-PolKv2 or nCas9-T5(D164A, E166A), 5 ng of mRNA encoding VPX, and 25 ng of mRNA encoding FEN1, along with a tgRNA in an 8-point 4-fold dilution series starting at 40 ng of tgRNA. The cells were lysed 72 hours post-treatment for NGS analysis as described in Example 1. Samples were run in triplicates, unless otherwise indicated.

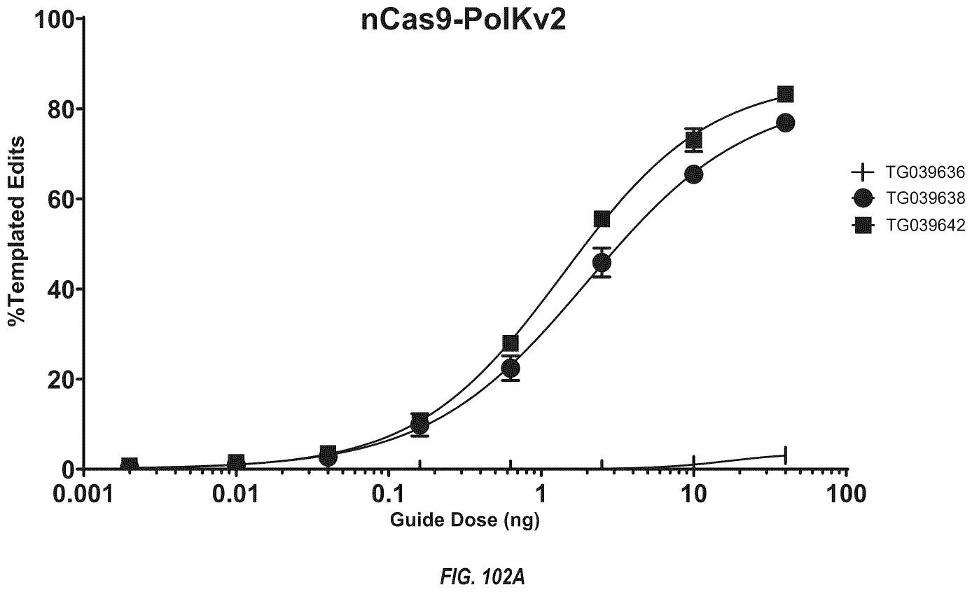

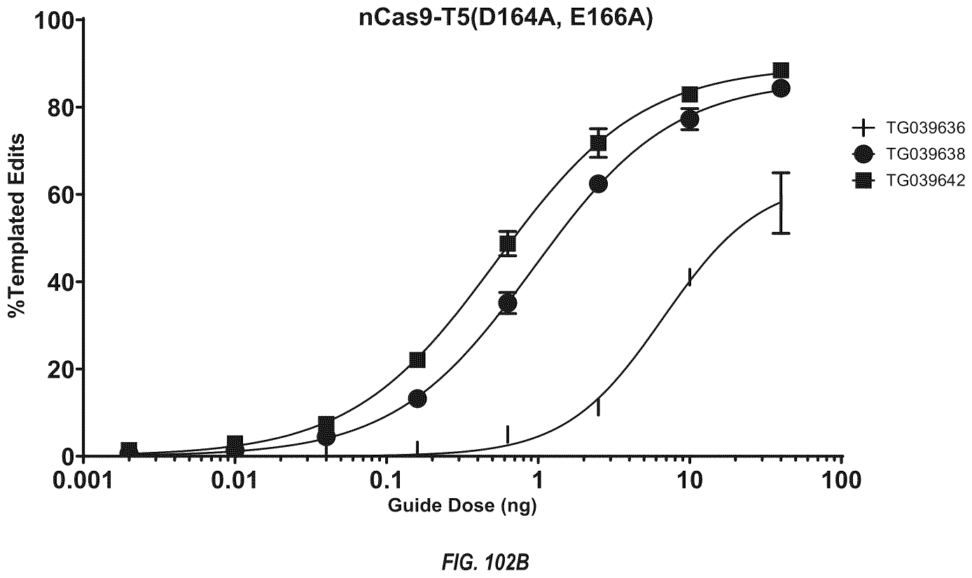

Table 122 and FIGS. 102A and B show the editing results at the VEGFA locus in PHH treated with tgRNAs comprising different dNTP/rNTP compositions and 2′Ome modifications in the spacer sequence.

TABLE 122
Editing results at the VEGFA locus in PHH treated with tgRNAs comprising different dNTP/rNTP
compositions and 2′OMe modifications in the spacer sequence, as described in Table 115.

| | | | tgRNA | | | | | | | | |
|---|---|---|---|---|---|---|---|---|---|---|---|
| mRNA ID | Guide ID | Edit Type | (ng) | 40 | 10 | 2.5 | 0.63 | 0.16 | 0.04 | 0.01 | 0.002 |
| nCas9- | TG039638 | % Templated | Mean | 76.90 | 65.43 | 45.87 | 22.42 | 9.84 | 2.68 | 0.87 | 0.37 |
| PolKv2 | | Edits | SD | 1.71 | 1.33 | 3.20 | 2.73 | 2.51 | 0.48 | 0.23 | 0.21 |
| | | % Templated + | Mean | 1.81 | 1.86 | 1.61 | 0.81 | 0.31 | 0.05 | 0.06 | 0.01 |
| | | Byproduct | SD | 0.21 | 0.17 | 0.39 | 0.29 | 0.27 | 0.03 | 0.06 | 0.02 |
| | | Edits | | | | | | | | | |
| | | % Indels | Mean | 1.02 | 0.86 | 0.78 | 0.29 | 0.16 | 0.04 | 0.03 | 0.01 |
| | | | SD | 0.19 | 0.03 | 0.18 | 0.09 | 0.10 | 0.04 | 0.03 | 0.00 |
| | TG039642 | % Templated | Mean | 83.27 | 73.08 | 55.55 | 27.95 | 10.55 | 3.49 | 1.48 | 0.74 |
| | | Edits | SD | 1.73 | 2.54 | 1.04 | 1.37 | 1.02 | 0.28 | 0.19 | 0.13 |
| | | % Templated + | Mean | 2.41 | 2.23 | 1.74 | 1.02 | 0.34 | 0.05 | 0.04 | 0.01 |
| | | Byproduct | SD | 0.36 | 0.07 | 0.15 | 0.32 | 0.10 | 0.04 | 0.05 | 0.01 |
| | | Edits | | | | | | | | | |
| | | % Indels | Mean | 1.36 | 1.10 | 0.98 | 0.55 | 0.19 | 0.04 | 0.03 | 0.01 |
| | | | SD | 0.11 | 0.42 | 0.15 | 0.04 | 0.11 | 0.02 | 0.03 | 0.01 |
| | TG039636 | % Templated | Mean | 3.02 | 1.01** | 0.10 | 0.02 | 0.01 | 0.00 | 0.00 | 0.09 |
| | | Edits | SD | 0.18 | 0.48** | 0.12 | 0.03 | 0.01 | 0.00 | 0.00 | 0.15 |
| | | % Templated + | Mean | 0.09 | 0.01** | 0.01 | 0.00 | 0.00 | 0.00 | 0.00 | 0.00 |
| | | Byproduct | SD | 0.07 | 0.01** | 0.01 | 0.00 | 0.00 | 0.00 | 0.00 | 0.00 |
| | | Edits | | | | | | | | | |
| | | % Indels | Mean | 0.07 | 0.00** | 0.01 | 0.01 | 0.00 | 0.00 | 0.01 | 0.01 |
| | | | SD | 0.06 | 0.00** | 0.01 | 0.01 | 0.00 | 0.00 | 0.01 | 0.00 |
| nCas9- | TG039638 | % Templated | Mean | 84.33 | 77.24 | 62.42 | 35.14 | 13.21 | 4.47 | 1.37 | 0.63 |
| T5(D164A, | | Edits | SD | 0.23 | 2.40 | 0.10 | 2.44 | 1.72 | 0.32 | 0.23 | 0.23 |
| E166A) | | % Templated + | Mean | 1.45 | 1.08 | 1.21 | 0.80 | 0.31 | 0.18 | 0.00 | 0.00 |
| | | Byproduct | SD | 0.17 | 0.34 | 0.24 | 0.23 | 0.09 | 0.13 | 0.00 | 0.00 |
| | | Edits | | | | | | | | | |
| | | % Indels | Mean | 1.66 | 1.55 | 1.52 | 1.02 | 0.46 | 0.20 | 0.01 | 0.01 |
| | | | SD | 0.24 | 0.49 | 0.34 | 0.20 | 0.08 | 0.12 | 0.02 | 0.01 |
| | TG039642 | % Templated | Mean | 88.45 | 82.88 | 71.76 | 48.75 | 22.09 | 7.40 | 2.94 | 1.43 |
| | | Edits | SD | 0.90 | 1.18 | 3.30 | 2.82 | 1.43 | 1.23 | 0.31 | 0.31 |
| | | % Templated + | Mean | 1.70 | 1.73 | 1.51 | 1.09 | 0.47 | 0.12 | 0.12 | 0.08 |
| | | Byproduct | SD | 0.35 | 0.24 | 0.58 | 0.08 | 0.16 | 0.09 | 0.12 | 0.02 |
| | | Edits | | | | | | | | | |
| | | % Indels | Mean | 2.13 | 2.29 | 2.67 | 1.66 | 0.88 | 0.23 | 0.21 | 0.12 |
| | | | SD | 0.46 | 0.09 | 0.84 | 0.59 | 0.26 | 0.02 | 0.00 | 0.01 |
| | TG039636 | % Templated | Mean | 58.01 | 41.04** | 11.26** | 4.99 | 1.45 | 0.36 | 0.04 | 0.02 |
| | | Edits | SD | 6.94 | 0.37** | 0.72** | 0.36 | 0.40 | 0.30 | 0.06 | 0.03 |
| | | % Templated + | Mean | 1.13 | 1.64** | 0.62 | 0.14 | 0.10 | 0.01 | 0.00 | 0.00 |
| | | Byproduct | SD | 0.72 | 0.47** | 0.02 | 0.04 | 0.10 | 0.01 | 0.00 | 0.00 |
| | | Edits | | | | | | | | | |
| | | % Indels | Mean | 1.51 | 2.28** | 0.75 | 0.28 | 0.05 | 0.01 | 0.00 | 0.01 |
| | | | SD | 0.83 | 0.26** | 0.20 | 0.09 | 0.05 | 0.01 | 0.00 | 0.00 |

**N = 2

### Example 58—Editing in a Single Dose Experiment at the TTR Locus in PCH Using tgRNAs with Different Template Lengths

In this Example, templated editing efficiency in primary cynomolgus hepatocytes (PCH) was assessed in a single-dose experiment with tgRNAs having a template sequence with various lengths (e.g., 14-20 nucleotides in length).

The editing efficiency of tgRNAs designed with the scaffold structure shown in FIG. 6F was evaluated at the TTR locus in Primary cynomolgus hepatocytes (PCH) treated with a nCas9-polymerase fusion protein, in the presence of Vpx (SEQ ID NO: 1501) and FEN-1 (SEQ ID NO: 1506). The tgRNAs comprised a spacer sequence targeting the TTR locus and a 3′ extension comprising a template sequence encoding a TG-to-AC edit, resulting in a STOP codon. The template sequences were designed with different lengths.

PCH (Gibco, Lot CY423) were prepared as described in Example 1, and plated on a collagen I coated 96-well plate (Corning, Cat. 354407) at a density of 50,000 cells/well. PCH were lipofected with a tgRNA selected from the tgRNAs described in Table 123, using pre-mixed lipid formulations with a molar composition of 50% Lipid A, 38% cholesterol, 9% DSPC, and 3% PEG2k-DMG as described in Example 1, at a fixed dose of 25 nM tgRNA per well.

TABLE 123
Exemplary tgRNAs designed with the scaffold structure shown in Fig. 6F, and with various
template lengths in the 3′ extension.

| | Template | |
|---|---|---|
| Guide ID | length (nt) | 3′extension sequence (template sequence is shown in bolded italics) |
| G037454 | 14 | dAdAdTdAdGdAdCdAdCdGdTdAdAdTdCUUACmUmGmGmAmAmG* |
| (SEQ ID | | mU (SEQ ID NO: 455) |
| NO: 586) | | |
| G037455 | 15 | dAdAdAdTdAdGdAdCdAdCdGdTdAdAdTdCUUACmUmGmGmAmAmG |
| (SEQ ID | | *mU (SEQ ID NO: 456) |
| NO: 587) | | |
| G037456 | 16 | dGdAdAdAdTdAdGdAdCdAdCdGdTdAdAdTdCUUACmUmGmGmAmA |
| (SEQ ID | | mG*mU (SEQ ID NO: 457) |
| NO: 588) | | |
| G037457 | 17 | dGdGdAdAdAdTdAdGdAdCdAdCdGdTdAdAdTdCUUACmUmGmGmA |
| (SEQ ID | | mAmG*mU (SEQ ID NO: 458) |
| NO: 589) | | |
| G037458 | 18 | dTdGdGdAdAdAdTdAdGdAdCdAdCdGdTdAdAdTdCUUACmUmGmGm |
| (SEQ ID | | AmAmG*mU (SEQ ID NO: 459) |
| NO: 590) | | |
| G037459 | 19 | dGdTdGdGdAdAdAdTdAdGdAdCdAdCdGdTdAdAdTdCUUACmUmGm |
| (SEQ ID | | GmAmAmG*mU (SEQ ID NO: 800) |
| NO: 591) | | |
| G037460 | 20 | dAdGdTdGdGdAdAdAdTdAdGdAdCdAdCdGdTdAdAdTdCUUACmUmG |
| (SEQ ID | | mGmAmAmG*mU (SEQ ID NO: 815) |
| NO: 592) | | |
| G037464 | 16 | dGdAdAdAdTdAdGdAdCdAdCdGdTdAdAdTdCUUACmUmGmGmAmA |
| (SEQ ID | | mGmU*mUU (SEQ ID NO: 838) |
| NO: 593) | | |
| G037465 | 16 | dGdAdAdAdTdAdGdAdCdAdCdGdTdAdAdTdCUUACmUmGmGmAmA |
| (SEQ ID | | mGmU*mU*U (SEQ ID NO: 839) |
| NO: 594) | | |
| G037466 | 16 | dGdAdAdAdTdAdGdAdCdAdCdGdTdAdAdTdCUUACmUmGmGmAmA |
| (SEQ ID | | mGmU*mU*mU (SEQ ID NO: 841) |
| NO: 595) | | |

Note:
*denotes that the nucleotide is linked to the next nucleotide with a phosphorothioate (PS bond), a lower case “m” denotes that the nucleotide is modified with 2′-O-Me, and a lowercase “d” denotes that the nucleotide is a DNA nucleotide.

LNPs were prepared as described in Example 1 with a molar ratio of 50% Lipid A, 38% cholesterol, 9% DSPC, and 3% PEG2k-DMG, and formulated with a lipid amine to RNA phosphate (N:P) molar ratio of about 6. Each LNP comprised a single mRNA species encoding one of the following proteins: nCas9-T5pol(v3) (SEQ ID NO: 1222), nCas9-PolK (1-526, GE411-412RV) (SEQ ID NO: 1232), Vpx (SEQ ID NO: 1501) or FEN-1 (SEQ ID NO: 1506). LNPs were incubated in media containing 10% fetal bovine serum (FBS) at 37° C. for 10 minutes. Post-incubation, the PCH were treated with LNPs comprising a mRNA encoding a nCas9-polymerase fusion protein selected from nCas9-T5pol(v3) (SEQ ID NO: 1222) or nCas9-PolK (1-526, GE411-412RV) (SEQ ID NO: 1232) (150 ng/well), a mRNA encoding Vpx (10 ng/well), and a mRNA encoding FEN-1 (10 ng/well).

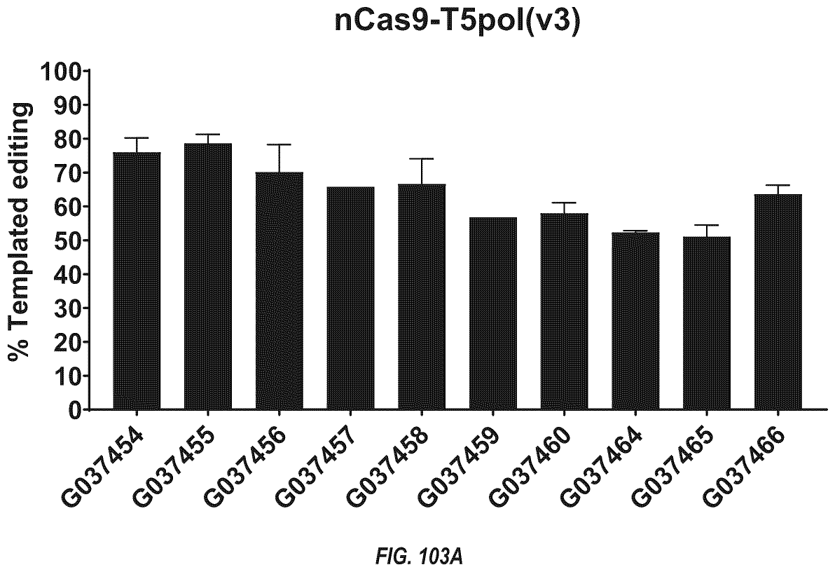

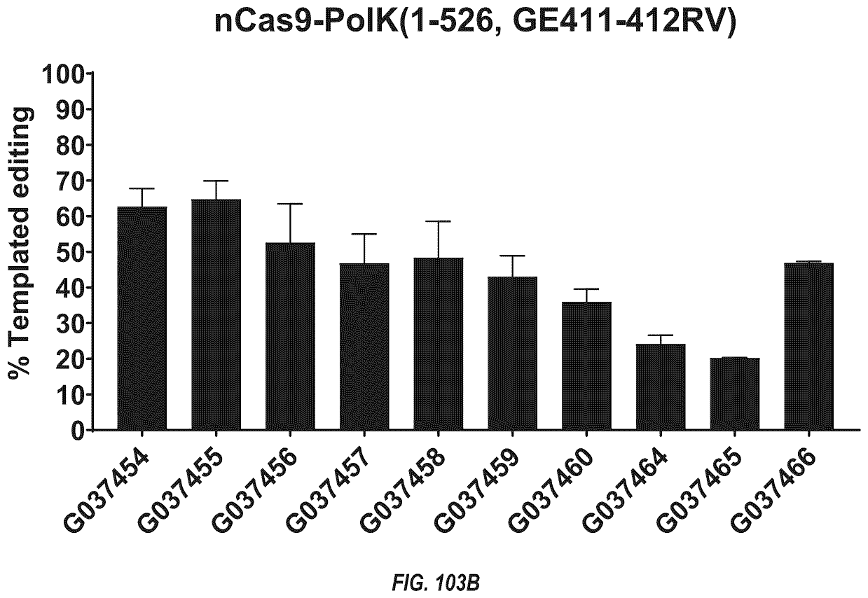

The cells were lysed 72 hours post-treatment for NGS analysis as described in Example 1. Samples were run in duplicates, unless otherwise indicated. Table 124 and FIGS. 103A-B show editing at the TTR locus in PCH treated with nCas9-T5pol(v3) (SEQ ID NO: 1222) or nCas9-PolK (1-526, GE411-412RV) (SEQ ID NO: 1232), in the presence of Vpx and FEN-1, and with tgRNAs designed with the scaffold structure shown in FIG. 6F, and with different template lengths.

TABLE 124
Editing at the TTR locus in PCH treated with nCas9-T5pol(v3) (SEQ ID NO: 1222) or nCas9-PolK(1-526, GE411-412RV) (SEQ ID NO: 1232), in the
presence of Vpx and FEN-1, and with tgRNAs designed with the scaffold structure shown in FIG. 6F, and with different template lengths.

| | | | G037454 | G037455 | G037456 | G037457 | G037458 | G037459 | G037460 | G037464 | G037465 | G037466 |
|---|---|---|---|---|---|---|---|---|---|---|---|---|
| nCas9- | % Templated | Mean | 76.00 | 78.65 | 70.15 | 65.8* | 66.65 | 56.80* | 58.00 | 52.35 | 51.10 | 63.65 |
| T5pol | Edits | SD | 4.24 | 2.62 | 8.13 | N/A | 7.42 | N/A | 3.11 | 0.49 | 3.39 | 2.62 |
| (v3) | % | Mean | 0.65 | 0.70 | 0.70 | 0.90* | 0.80 | 0.70* | 0.40 | 0.55 | 0.40 | 0.50 |
| | Templated + | SD | 0.07 | 0.14 | 0.00 | N/A | 0.14 | N/A | 0.14 | 0.07 | 0.14 | 0.00 |
| | Byproduct | | | | | | | | | | | |
| | Edits | | | | | | | | | | | |
| | % Indels | Mean | 0.30 | 0.20 | 0.35 | 0.25 | 0.25 | 0.70 | 0.30 | 0.30 | 0.20 | 0.30 |
| | | SD | 0.00 | 0.00 | 0.07 | 0.35 | 0.07 | 0.42 | 0.00 | 0.00 | 0.14 | 0.14 |
| nCas9- | % Templated | Mean | 62.70 | 64.75 | 52.60 | 46.80 | 48.40 | 43.05 | 36.00 | 24.20 | 20.25 | 46.90 |
| PolK | Edits | SD | 5.09 | 5.16 | 10.89 | 8.20 | 10.18 | 5.87 | 3.54 | 2.40 | 0.07 | 0.42 |
| (1- | % | Mean | 1.20 | 1.70 | 1.75 | 1.40 | 1.60 | 1.60 | 1.60 | 0.35 | 0.45 | 1.20 |
| 526, | Templated + | SD | 0.28 | 0.14 | 0.07 | 0.14 | 0.42 | 0.28 | 0.28 | 0.07 | 0.07 | 0.00 |
| GE411- | Byproduct | | | | | | | | | | | |
| 412RV) | Edits | | | | | | | | | | | |
| | % Indels | Mean | 0.15 | 0.30 | 0.35 | 0.20 | 0.25 | 0.20 | 0.20 | 0.05 | 0.15 | 0.20 |
| | | SD | 0.07 | 0.14 | 0.21 | 0.00 | 0.07 | 0.14 | 0.14 | 0.07 | 0.07 | 0.14 |

*N = 1

### Example 59—Editing in a Dose Response Experiment at the TTR Locus in PCH Using tgRNAs with Different Template Lengths

In this Example, templated editing efficiency in primary cynomolgus hepatocytes (PCH) was assessed in a dose-response experiment with tgRNAs having a template sequence with different lengths (e.g., 14-20 nucleotides in length).

The editing efficiency of tgRNAs designed with the scaffold structure shown in FIG. 6F was evaluated at the TTR locus in Primary cynomolgus hepatocytes (PCH) treated with a nCas9-polymerase fusion protein, in the presence of Vpx (SEQ ID NO: 1501) and FEN-1 (SEQ ID NO: 1506), in a dose response experiment. The tgRNAs comprised a spacer sequence targeting the TTR locus and a 3′ extension comprising a template sequence encoding a TG-to-AC edit, resulting in a STOP codon. The template sequences were designed with different lengths.

PCH (Gibco, Lot CY423) were prepared as described in Example 1, and plated on a collagen I coated 96-well plate (Corning, Cat. 354407) at a density of 50,000 cells/well. PCH were lipofected using pre-mixed lipid formulations with a molar composition of 50% Lipid A, 38% cholesterol, 9% DSPC, and 3% PEG2k-DMG as described in Example 1, and comprising a tgRNA selected from the tgRNAs described in Table 123, a mRNA encoding a nCas9-T5pol(v2) fusion protein (SEQ ID NO: 1217), a mRNA encoding Vpx (SEQ ID NO: 1501), and a mRNA encoding FEN-1 (SEQ ID NO: 1506), in a mass ratio of nCas9-T5pol(v2):tgRNA: FEN-1:Vpx of 6:3:1:1. Briefly, cells were incubated at 37° C., 5% CO₂ for 24 hours prior to treatment with the lipid nucleic acid mixtures. Post-incubation, the lipid nucleic acid mixtures were added to the cells in a 7-point 3-fold dilution series starting at 800 ng total RNA.

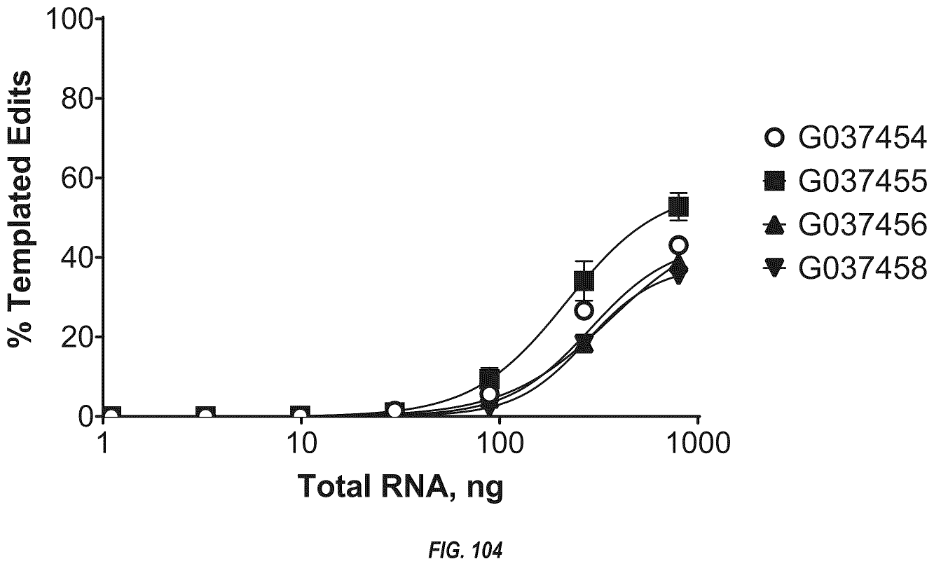

The cells were lysed 72 hours post-treatment for NGS analysis as described in Example 1. Samples were run in duplicates. Table 125 and FIG. 104 show editing at the TTR locus in PCH treated with nCas9-T5pol(v2), in the presence of Vpx and FEN-1, and with tgRNAs designed with the scaffold structure shown in FIG. 6F, and with different template lengths.

TABLE 125
Editing at the TTR locus in PCH treated with nCas9-T5pol(v2), in the presence
of Vpx and FEN-1, and with tgRNAs designed with the scaffold structure shown
in FIG. 6F, and with different template lengths, as described in Table 123

| | | | | Total RNA load (ng) | | | | | | | |
|---|---|---|---|---|---|---|---|---|---|---|---|
| mRNA ID | Guide ID | | | 800 | 266.67 | 88.89 | 29.63 | 9.88 | 3.29 | 1.1 | 0 |
| nCas9-T5pol(v2) | G037454 | % Templated | Mean | 43.10 | 26.65 | 5.65 | 1.45 | 0.15 | 0.05 | 0.00 | 0.00 |
| | | Edits | SD | 0.57 | 0.64 | 0.21 | 0.92 | 0.07 | 0.07 | 0.00 | 0.00 |
| | | % Templated + | Mean | 0.55 | 0.25 | 0.05 | 0.00 | 0.00 | 0.00 | 0.00 | 0.00 |
| | | Byproduct Edits | SD | 0.21 | 0.07 | 0.07 | 0.00 | 0.00 | 0.00 | 0.00 | 0.00 |
| | | % Indels | Mean | 0.10 | 0.10 | 0.00 | 0.00 | 0.00 | 0.00 | 0.00 | 0.00 |
| | | | SD | 0.00 | 0.00 | 0.00 | 0.00 | 0.00 | 0.00 | 0.00 | 0.00 |
| | G037455 | % Templated | Mean | 52.75 | 34.10 | 9.40 | 1.05 | 0.20 | 0.00 | 0.00 | 0.00 |
| | | Edits | SD | 3.46 | 4.95 | 2.83 | 0.07 | 0.14 | 0.00 | 0.00 | 0.00 |
| | | % Templated + | Mean | 0.50 | 0.15 | 0.10 | 0.00 | 0.00 | 0.05 | 0.00 | 0.00 |
| | | Byproduct Edits | SD | 0.28 | 0.07 | 0.00 | 0.00 | 0.00 | 0.07 | 0.00 | 0.00 |
| | | % Indels | Mean | 0.25 | 0.05 | 0.05 | 0.00 | 0.00 | 0.00 | 0.00 | 0.00 |
| | | | SD | 0.07 | 0.07 | 0.07 | 0.00 | 0.00 | 0.00 | 0.00 | 0.00 |
| | G037456 | % Templated | Mean | 38.55 | 18.45 | 4.50 | 0.35 | 0.15 | 0.00 | 0.00 | 0.00 |
| | | Edits | SD | 1.20 | 0.21 | 0.14 | 0.21 | 0.21 | 0.00 | 0.00 | 0.00 |
| | | % Templated + | Mean | 0.55 | 0.25 | 0.05 | 0.00 | 0.00 | 0.00 | 0.00 | 0.00 |
| | | Byproduct Edits | SD | 0.07 | 0.07 | 0.07 | 0.00 | 0.00 | 0.00 | 0.00 | 0.00 |
| | | % Indels | Mean | 0.15 | 0.10 | 0.00 | 0.00 | 0.00 | 0.00 | 0.00 | 0.00 |
| | | | SD | 0.07 | 0.14 | 0.00 | 0.00 | 0.00 | 0.00 | 0.00 | 0.00 |
| | G037458 | % Templated | Mean | 35.40 | 18.30 | 2.30 | 0.35 | 0.10 | 0.00 | 0.00 | 0.00 |
| | | Edits | SD | 0.28 | 0.42 | 0.42 | 0.07 | 0.14 | 0.00 | 0.00 | 0.00 |
| | | % Templated + | Mean | 0.50 | 0.20 | 0.00 | 0.00 | 0.00 | 0.00 | 0.00 | 0.00 |
| | | Byproduct Edits | SD | 0.14 | 0.14 | 0.00 | 0.00 | 0.00 | 0.00 | 0.00 | 0.00 |
| | | % Indels | Mean | 0.25 | 0.10 | 0.00 | 0.00 | 0.00 | 0.00 | 0.00 | 0.00 |
| | | | SD | 0.21 | 0.00 | 0.00 | 0.00 | 0.00 | 0.00 | 0.00 | 0.00 |

### Example 60—Editing in PCH with nCas9-Polymerase, Editing Enhancers, and tgRNAs with Different Template Lengths

The editing efficiency of tgRNAs designed with the scaffold structure shown in FIG. 6F was evaluated at the TTR locus in Primary cynomolgus hepatocytes (PCH) treated with a nCas9-polymerase fusion protein, in the presence of Vpx (SEQ ID NO: 1501) and FEN-1 (SEQ ID NO: 1506). The tgRNAs comprised a spacer sequence targeting the TTR locus and a 3′ extension comprising a template sequence encoding a TG-to-AC edit, resulting in a STOP codon. The template sequences were designed with different lengths.

PCH (Gibco, Lot CY423) were prepared as described in Example 1, and plated on a collagen I coated 96-well plate (Corning, Cat. 354407) at a density of 50,000 cells/well. LNPs were prepared as described in Example 1 with a molar ratio of 50% Lipid A, 38% cholesterol, 9% DSPC, and 3% PEG2k-DMG, and formulated with a lipid amine to RNA phosphate (N:P) molar ratio of about 6. Each LNP comprised a single RNA species corresponding to a tgRNA (G037454, or G037455), or a mRNA encoding one of the following proteins: nCas9-T5pol(v2) (SEQ ID NO: 1217), nCas9-PolK (1-526, GE411-412RV) (SEQ ID NO: 1232), Vpx, or FEN-1. LNPs were incubated in media containing 10% fetal bovine serum (FBS) at 37° C. for 10 minutes. Post-incubation, the PCH were treated with LNPs comprising a mRNA encoding nCas9-T5pol(v2) or nCas9-PolK (1-526, GE411-412RV) (200 ng/well), a mRNA encoding Vpx (25 ng/well), and a mRNA encoding FEN-1 (25 ng/well). The cells were also treated with a LNP comprising a tgRNA in a 8-point 3-fold dilution series starting at 300 ng tgRNA/well.

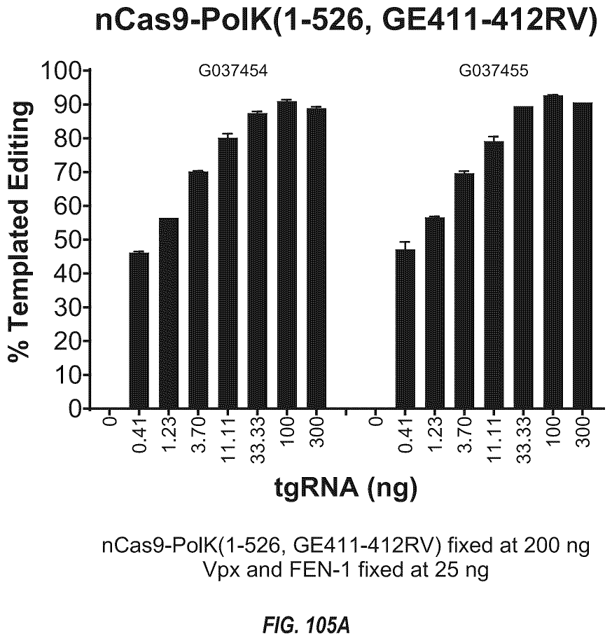

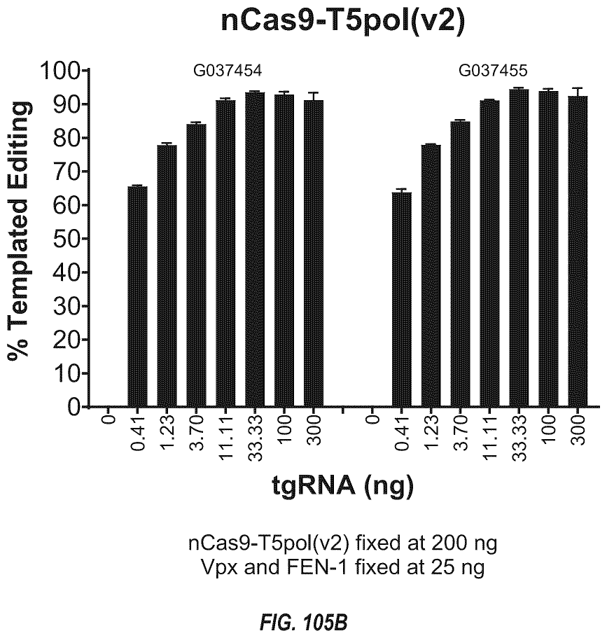

The cells were lysed 72 hours post-treatment for NGS analysis as described in Example 1. An additional computational analysis was conducted on the NGS data to quantify reads containing a stop codon (i.e., percent reads with stop codon relative to total reads). Samples were run in duplicates. Table 126 and FIGS. 105A-B show editing at the TTR locus in PCH treated with nCas9-T5pol(v2) (SEQ ID NO: 1217) or nCas9-PolK(1-526, GE411-412RV) (SEQ ID NO: 1232), in the presence of Vpx and FEN-1, and with tgRNAs designed with the scaffold structure shown in FIG. 6F, and with different template lengths.

TABLE 126
Editing at the TTR locus in PCH treated with nCas9-T5pol(v2) or nCas9-PolK(1-526, GE411-
412RV), in the presence of Vpx and FEN-1, and with tgRNAs designed with the scaffold structure
shown in FIG. 6F, and with different template lengths, as described in Table 123.

| | | | | | % Templated + | | | | | |
|---|---|---|---|---|---|---|---|---|---|---|
| | | Guide | % Templated Edits | | Byproduct Edits | | % Indels | | STOP codon % | |
| mRNA ID | Guide ID | dose (ng) | Mean | SD | Mean | SD | Mean | SD | Mean | SD |
| nCas9- | G037454 | 300 | 88.90 | 0.42 | 1.65 | 0.35 | 0.20 | 0.00 | 90.30 | 0.14 |
| PolK(1-526, | | 100 | 91.00 | 0.42 | 1.40 | 0.00 | 0.25 | 0.07 | 92.30 | 0.42 |
| GE411- | | 33.33 | 87.45 | 0.49 | 1.90 | 0.71 | 0.30 | 0.00 | 89.05 | 0.21 |
| 412RV) | | 11.11 | 80.15 | 1.20 | 1.70 | 0.14 | 0.25 | 0.21 | 81.65 | 1.20 |
| | | 3.7 | 70.20 | 0.14 | 1.55 | 0.07 | 0.25 | 0.07 | 71.60 | 0.14 |
| | | 1.23 | 56.50 | 0.00 | 1.45 | 0.35 | 0.25 | 0.07 | 57.85 | 0.35 |
| | | 0.41 | 46.15 | 0.35 | 1.05 | 0.07 | 0.10 | 0.00 | 47.20 | 0.28 |
| | | 0 | 0.00 | 0.00 | 0.00 | 0.00 | 0.00 | 0.00 | 0.20 | 0.00 |
| | G037455 | 300 | 90.60 | 0.00 | 1.70 | 0.28 | 0.25 | 0.21 | 92.05 | 0.21 |
| | | 100 | 92.70 | 0.14 | 1.75 | 0.49 | 0.30 | 0.00 | 94.20 | 0.42 |
| | | 33.33 | 89.50 | 0.00 | 2.05 | 0.07 | 0.30 | 0.14 | 91.20 | 0.14 |
| | | 11.11 | 79.15 | 1.34 | 2.10 | 0.42 | 0.30 | 0.14 | 81.00 | 1.98 |
| | | 3.7 | 69.65 | 0.64 | 1.55 | 0.07 | 0.20 | 0.00 | 71.15 | 0.64 |
| | | 1.23 | 56.60 | 0.28 | 1.40 | 0.14 | 0.25 | 0.07 | 57.90 | 0.28 |
| | | 0.41 | 47.15 | 2.19 | 1.15 | 0.07 | 0.15 | 0.07 | 48.30 | 2.26 |
| | | 0 | 0.00 | 0.00 | 0.00 | 0.00 | 0.00 | 0.00 | 0.25 | 0.07 |
| nCas9- | G037454 | 300 | 91.25 | 2.19 | 1.25 | 0.07 | 0.20 | 0.00 | 92.45 | 2.19 |
| T5pol(v2) | | 100 | 92.90 | 0.85 | 1.20 | 0.14 | 0.35 | 0.07 | 93.80 | 0.99 |
| | | 33.33 | 93.55 | 0.35 | 1.40 | 0.14 | 0.35 | 0.07 | 94.50 | 0.28 |
| | | 11.11 | 91.20 | 0.57 | 1.15 | 0.07 | 0.25 | 0.07 | 92.30 | 0.57 |
| | | 3.7 | 84.10 | 0.57 | 1.15 | 0.07 | 0.30 | 0.00 | 85.05 | 0.64 |
| | | 1.23 | 77.85 | 0.64 | 1.25 | 0.07 | 0.45 | 0.21 | 78.80 | 0.85 |
| | | 0.41 | 65.55 | 0.35 | 0.90 | 0.00 | 0.25 | 0.07 | 66.30 | 0.28 |
| | | 0 | 0.00 | 0.00 | 0.00 | 0.00 | 0.00 | 0.00 | 0.30 | 0.00 |
| | G037455 | 300 | 92.45 | 2.33 | 1.40 | 0.14 | 0.35 | 0.07 | 93.65 | 2.33 |
| | | 100 | 93.95 | 0.64 | 1.35 | 0.35 | 0.40 | 0.42 | 94.95 | 0.64 |
| | | 33.33 | 94.45 | 0.49 | 1.25 | 0.07 | 0.35 | 0.07 | 95.55 | 0.64 |
| | | 11.11 | 91.15 | 0.21 | 1.05 | 0.21 | 0.30 | 0.14 | 92.00 | 0.28 |
| | | 3.7 | 84.85 | 0.49 | 1.25 | 0.07 | 0.45 | 0.07 | 85.80 | 0.28 |
| | | 1.23 | 77.95 | 0.21 | 1.15 | 0.07 | 0.30 | 0.00 | 78.90 | 0.42 |
| | | 0.41 | 63.80 | 0.99 | 0.75 | 0.07 | 0.20 | 0.00 | 64.55 | 0.92 |
| | | 0 | 0.00 | 0.00 | 0.00 | 0.00 | 0.00 | 0.00 | 0.25 | 0.07 |

### Example 61—Production of 117-Nt tgRNA Through Ligation of Two Oligonucleotides

In this Example, a scalable production method was assessed for producing a template guide RNA (tgRNA) by ligation of two oligonucleotides (with the aid of a splint oligonucleotide).

Scalable Production of a 117-Nt tgRNA Through Ligation of Two Oligonucleotides

A method for producing a template guide RNA (tgRNA) by ligation was developed to produce a tgRNA with a length of 117 nucleotides at a 200 milligram scale, by ligating a 5′end split fragment and a 3′end split fragment of the desired final tgRNA product as shown in FIG. 106A.

The oligonucleotides listed in Table 127 were obtained from Axolabs (Kulmbach, Germany). These comprised two fragment oligonucleotides, and a 27-mer splint DNA oligonucleotide that was used to ligate the two fragment oligonucleotides into the final full-length guide RNA product (G039267).

TABLE 127
Exemplary oligonucleotides used for the guide ligation procedure described herein.

| | | | Con- | |
|---|---|---|---|---|
| | | | centration | Purity |
| | | Molecular | in | (%) |
| | | Weight | solution | measured |
| Description | Sequence | (g/mol) | (uM) | by UPLC |
| 27-mer splint | dGdTdGdAdTdAdAdCdGdGdAdCdTdAdGdCdCdTdTdAd | 8272.4 | 200.1 | 77.4 |
| DNA | TdTdTdTdAdAdC (SEQ ID NO: 474) | | | |
| 5′end ligation | mA*mA*mG*UGACUUCCAGUAAGAUUmGUUUUAGmAmGmCm | 18131.7 | 100.1 | 77.8 |
| split fragment | UmAmGmAmAmAmUmAmGmCmAAGUUmAAmAAmUmAmAGm | | | |
| | GC (SEQ ID NO: 475) | | | |
| 3′end ligation | pUmAGUCmCmGUUAUmCAmCGmAmAmAmGGmGmCmAmCmC | 20244.4 | 99.9 | 85.6 |
| split fragment | mGmAGUCmGmGmUmGmCdAdAdAdTdAdGdAdCdAdCdGdT | | | |
| | dAdAdTdCUUACmUmGmGmAmAmG*mU | | | |
| | (SEQ ID NO: 476) | | | |

Note:
*denotes that the nucleotide is linked to the next nucleotide with a phosphorothioate (PS bond), a lower case “m” denotes that the nucleotide is modified with 2′-O-Me, and a lowercase “d” denotes that the nucleotide is a DNA nucleotide.

The three oligonucleotides were thawed and mixed together in a 5′end split fragment: 3′end split fragment: splint DNA volume ratio of 1:1:0.5 for 5 minutes using a stir bar (total volume: 138 mL). Then the mixture was aliquoted into 10 conical tubes (Eppendorf™, 50 mL) that were loaded onto an Eppendorf ThermoMixer® and shaken at 300 rpm and 95° C. for 15 minutes. The temperature was then set to 4° C. for 40 minutes with shaking at 300 rpm with the lid removed. The 10 aliquots were brought back to room temperature and mixed together with 5 mL of T4 RNA ligase 2 (4.55 mg/ml), 20 mL of a 10×RNA ligase 2 buffer (500 mM Tris-HCl (pH7.5), 20 mM MgCl₂, 4 mM ATP, 10 mM DTT), 2.17 mL of a 40U/uL RNase inhibitor (Roche, cat. #03247058103), 35 mL of water (Corning, 46-000C1), for 5 minutes using a stir bar. The mixture was then aliquoted into 10 conical tubes (Eppendorf™, 50 mL) that were loaded onto the ThermoMixer® and shaken at 300 rpm at 37° C. for 40 minutes.

The mixture was purified by precipitation as follows. Four liters of an RNA precipitation buffer were prepared by mixing 400 mL of 250 mM sodium citrate buffer, pH 6.5, with 26.67 mL of 30% (v/v) N-lauroylsarcosine sodium, 1840 g of guanidine thiocyanate, and 1733 mL of water (Corning, 46-000Cl). The 10 aliquots were mixed together in a 5 L-bottle, and 750 mL of the prepared RNA precipitation buffer were added. The mixture was stirred for 5 minutes. Then, 2700 mL of 100% ethanol (Fisher Scientific, BP2818-500) were added and the mixture was stirred again for 5 minutes before being stored at 4° C. The next day, the mixture was divided in 4 aliquots that were each passed through a 90-mm diameter, 0.2 μm-pore filter (ThermoFisher Scientific™ Nalgene™ Rapid-Flow™ Sterile Disposable Filter Units with PES Membranes; cat. #0974103). The mixture was allowed to flow through the filter by gravity for 20 minutes, then vacuum was added to complete the filtering process. The filters were washed twice with 133 mL of a solution composed of 25% RNA precipitation buffer (prepared as described above) and 75% ethanol (by volume). Then, the filters were washed three times with 200 mL of a 90%/10% ethanol/water (by volume). The filtrate was discarded. The first filter was eluted with 80 mL of water, and the resulting eluate (E1) was used to re-elute the other three filters. Then, the first filter was eluted again with 80 mL of water, and the resulting second eluate (E2) was used to re-elute the other three filters. E1 and E2 were collected and mixed together for further analysis.

The procedure was repeated three times, generating three batches of eluates. One sample from each batch was analyzed using Nanodrop (ThermoFisher Scientific, ND8000). The results of the analysis are summarized in Table 128.

TABLE 128
Nanodrop analysis of the filtered material after ligation. Yield
comprises the splint DNA oligonucleotide, which explains why it
is greater than 200 mg. Recovery (%) is calculated according to
the following formula: 100% × Yield (mg)/Start material (mg).

| Start material | Scale (mg) | Yield (mg) | Recovery (%) |
|---|---|---|---|
| Batch 1 | 200 | 277 | >90 |
| Batch 2 | 200 | 270 | >90 |
| Batch 3 | 200 | 270 | >90 |

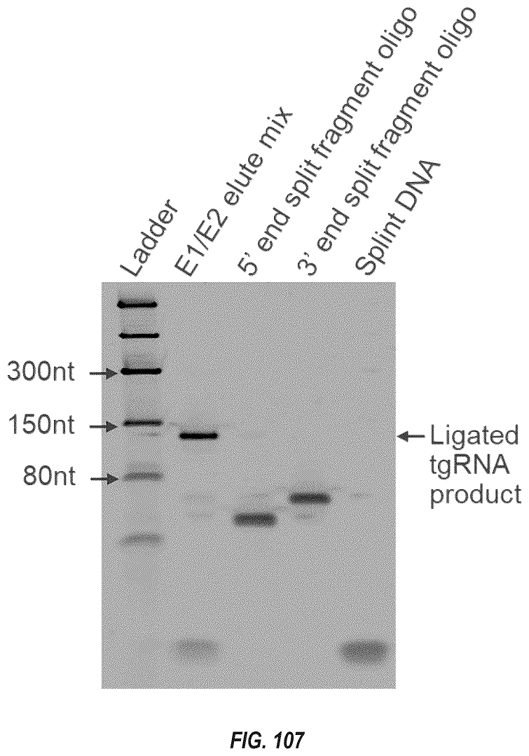

A sample from each eluate batch was assayed against samples from the three initial oligonucleotides on a 15% TBE-Urea gel (Invitrogen, EC68852BOX) using RNA loading buffer II (Invitrogen, AM8546G) and a low-range ssRNA ladder (NEB, N0364S). Ligation efficiency was determined based on the gel lines corresponding to the ligated tgRNA product, relative to the lines corresponding to traces of the initial three oligonucleotides in each eluate batch sample. The gel analysis is summarized for all three batches in Table 129, and shown for one batch (Batch 1) in FIG. 107.

TABLE 129
Ligation efficiency from the gel analysis of the eluate
mix following purification by precipitation.

| Start material | Ligation efficiency (%) |
|---|---|
| Batch 1 | 92.9 |
| Batch 2 | 92.2 |
| Batch 3 | 92.4 |

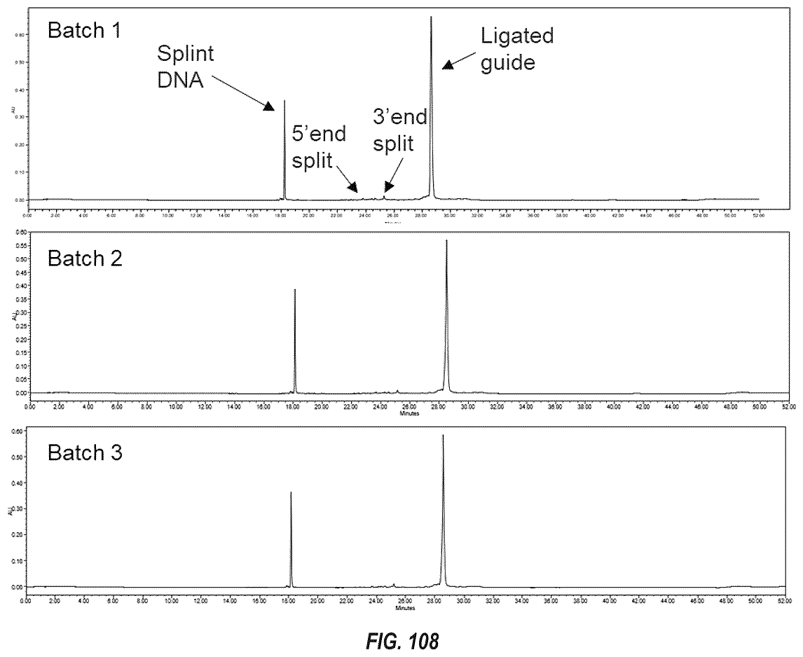

A sample from each eluate batch was further assessed with ultra-performance liquid chromatography (UPLC) to confirm the production of the ligated tgRNA. Purity of the ligated tgRNA product was determined using ion pairing reversed phase ultra performance liquid chromatography (IPRP-UPLC) with UV detection at 260 nm. The separation was achieved using a Waters ACQUITY UPLC Peptide BEH C18 Column, 300 Å, 1.7 μm, 2.1 mm×150 mm column with a flow rate of 0.3 mL/min. Column temperature was set at 75° C. Mobile phase A was 0.2% hexylamine and 2% 1,1,1,3,3,3-hexafluoroisopropanol in water. Mobile phase B was 50% methanol and 50% acetonitrile. The gradient was increased from 10% to 35% mobile phase B from 0 to 20 min, then from 35% to 50% mobile phase B from 20 to 45 min, then the column was re-equilibrated at 10% mobile phase B for 6 minutes. The purity of the final tgRNA product was determined as the fraction of total peak area (% AUC, also referred to as % area) attributed to the corresponding signal in the UPLC-UV chromatogram. The UPLC-UV chromatogram for each batch is shown in FIG. 108, and the purity analysis is shown in Table 130.

TABLE 130
Purity analysis from the UPLC-UV chromatograms of the eluate
batches obtained after purification by precipitation.

| Start material | Crude purity (%) | Purity without splint DNA (%) |
|---|---|---|
| Batch 1 | 61.1 | 72.7 |
| Batch 2 | 63.6 | 76.4 |
| Batch 3 | 62.8 | 76.8 |

Purification of the Crude Ligated 117-Nt tgRNA Using RP-HPLC

After the purification by precipitation as described above, each of the three collected eluate batches was split into two halves, thus creating a total of six crude ligated tgRNA batches for further processing. The optical density (OD) was measured for a sample from each of the six batches using a SpectraMax® Pro reader at 260 nm wavelength. The concentration of the crude ligated tgRNA in each sample was determined, based on the OD at 260 nm and the extinction coefficient of the desired tgRNA full-length sequence. The approximate amount (in milligrams) of oligonucleotide in each sample was calculated based on the determined concentration of the crude ligated tgRNA.

Then, each of the six batches was purified via reversed-phase high-performance liquid chromatography (RP-HPLC), as follows, using a GE ÄKTA Pure 150 system with a Timberline Mobile Phase Heating system set to 75° C. and flow rate of 2.6 mL/min. The reversed-phase (RP) purification column used was a Cytiva 16 mL Source 30 RPC 200 mm×10 mm in a stainless-steel vessel. Mobile phase A (Buffer A) comprised 100 mM triethylamine acetate (TEAA) in 95:5 Water: Acetonitrile pH ~7, and mobile phase B (Buffer B) comprised 100 mM TEAA in 65:35 Water: Acetonitrile pH ~7. First, the column was sanitized with 100% Buffer B for 4 column volumes (CV) followed by equilibration using 100% Buffer A for 4 CVs prior to loading the crude sample. Each batch of crude ligated tgRNA was diluted to ~50 OD/mL of column resin in 100 mM TEAA, via the addition of an adequate volume of 0.2 M TEAA and water, prior to application onto the column. After the sample application, the column was washed with 100% Buffer A for 3 CVs to remove any species not bound to the RP column, followed by an isocratic step at 5% Buffer B for 2 CVs before the start of the elution gradient. The elution gradient comprised a first linear increase to 20% Buffer B over 7.3 CVs, followed by an isocratic step at the same buffer concentration for 3.3 CVs to maximize the separation of the splint DNA species from the crude ligated tgRNA material, then ramping up to 40% Buffer B over 19.5 CVs to separate the unligated 5′end and 3′end split fragments from the full-length product (FLP, i.e. the desired ligated tgRNA product). An additional volume of 7 mL of the 40% Buffer B/60% Buffer A mixture was passed through the column at the end of this stage to account for any dead volume in the system. Then, the gradient was linearly increased to 100% Buffer B over 5 CVs, and a volume of 7 mL of 100% Buffer B was passed through the column to account for dead volume. The column was rinsed with 100% Buffer B for 3CVs to ensure that all oligonucleotide species were eluted from the RP column. Finally, the column was re-equilibrated using 100% Buffer A for 3 CVs.

During the elution gradient described above, fractions of the eluted material were automatically collected based on a 280 nm UV threshold. The collected fractions were then analyzed using OD readout, as described above, to determine yield, and liquid chromatography-mass spectrometry (LC-MS) was used for molecular weight confirmation. Based on the LC-MS data, mock pools were created from selected fractions to determine the fraction range containing the highest yield and proportion of FLP relative to other non-FLP species. The mock pools were analyzed via LC-MS for molecular weight confirmation, as well as UPLC-UV, as described above, to determine purity. Once the mock pool with the highest yield and proportion of FLP (based on LC-MS data) was determined, the appropriate fractions were combined for a final pool from that RP-HPLC run.

Each final pool obtained after the six batch purifications was analyzed for yield (using optical density as described above), molecular weight confirmation (using LC-MS) and % FLP purity (using UPLC-UV). Then, each pool was subjected to sodium salt (IM NaCl) exchange overnight (16 hr) via tangential flow filtration (TFF) using a ÄKTA Flux system with a Sartorius Sartocon Slice 200 ECO Hydrosart 10 kDa 200 cm² Filter Cassette for Ultrafiltration to remove the reversed-phase ion-pairing TEAA salt. Then, the sodium salt was replaced with 100% MilliQ water to allow for the pools to undergo buffer exchange and desalting until conductivity reached ~20 uS. All six purified and desalted final products were lyophilized to a white/off-white powder before being resuspended in cell culture grade water (Corning, 25-055-CM) to ~150 μM concentration each, based on UV readout at 260 nm. The resulting six solutions were combined, and concentration was brought down to 100 μM with the addition of more nuclease-free water.

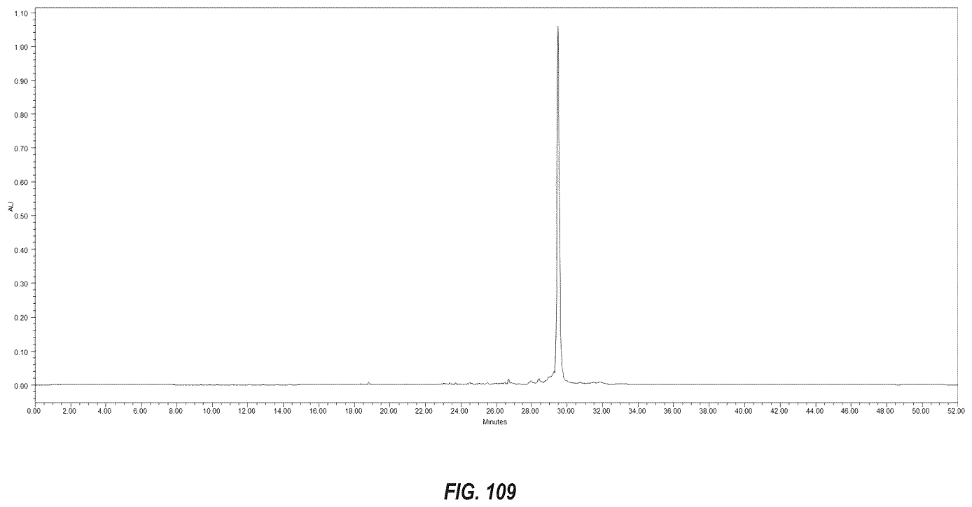

The final ligated 117-nucleotide tgRNA material was analyzed via UPLC to determine final purity, and via LC-MS to confirm molecular weight. FIG. 109 shows the resulting UPLC-UV chromatogram. Concentration and yield were determined based on optical density calculations at 260 nm, using the method described above. Table 131 shows the measured yield, as well as the purity of the ligated tgRNA measured by UPLC-UV.

TABLE 131
Yield (measured by optical density) and purity
(measured by UPLC-UV) of the final ligated
tgRNA product following RP-HPLC purification.

| Sample | Yield (mg) | % purity |
|---|---|---|
| Ligated tgRNA product (G039267) | 300 | 79.1 |

### Example 62-Production of tgRNA Longer than 150 Nucleotides Through a Two-Site Ligation Approach

Production of a 179-Nt tgRNA Through Ligation of Three Oligonucleotides

A two-site guide ligation method was developed to produce a tgRNA with a length of 179 nucleotides at a 5 milligram scale, by ligating three split fragments of the desired final tgRNA product as shown in FIGS. 106B-C.

The split fragment oligonucleotides listed in Table 132 were obtained from Genscript (Piscataway, NJ, USA). The splint DNA oligonucleotides listed in Table 132 were obtained from IDT (Coralville, IA, USA). The two splint DNA oligonucleotides were used to ligate the three fragment oligonucleotides into the final full-length guide RNA product (G038241; SEQ ID NO: 598).

TABLE 132
Exemplary oligonucleotides used for the guide ligation procedure described herein.

| | | Molecular | Concentration | Purity (%) |
|---|---|---|---|---|
| | | Weight | in solution | measured by |
| Description | Sequence | (g/mol) | (uM) | UPLC |
| 5′end | mG*mA*mU*GUCUGCAGGCCAGAU | 19971.91 | 100 | 53.7 |
| ligation split | GAGUUUUAGAmGmCmUmAmGmA | | | |
| fragment | mAmAmUmAmGmCAAGUUAAAAUA | | | |
| (SEQ ID | AGGCUAGUCC | | | |
| NO: 469) | | | | |
| middle | pGUUAUCACGAAAGGGCACCGAGU | 18479.12 | 100 | 53.6 |
| ligation split | CGGmUmGmCmCmAmCmAmGmUm | | | |
| fragment | GmUmGmUmCmCmCmUmCmUdGdA | | | |
| (SEQ ID | dCdAdAdTdGdTdGdCdCdA | | | |
| NO: 470) | | | | |
| 3′end | pdTdCdTdGdGdAdGdCdCdCdTdCdAd | 18954.85 | 100 | 80.3 |
| ligation split | AdTdGdAdTdCdCdTdGdAdCdGdAdCd | | | |
| fragment | GdGdAdGdAdCdCdGdCdCdGdTdCdGd | | | |
| (SEQ ID | TdCdGdAdCdAdAdGdCdCdTCUGGCm | | | |
| NO: 471) | CmUmG*mC | | | |
| Splint DNA- | CTTTCGTGATAACGGACTAGCCTTA | 8319.4 | 400 | Not available |
| 1 (SEQ ID | TT | | | |
| NO: 472) | | | | |
| Splint DNA- | TGAGGGCTCCAGATGGCACATTGT | 8419.44 | 400 | Not available |
| 2 (SEQ ID | CAG | | | |
| NO: 473) | | | | |

Note:
*denotes that the nucleotide is linked to the next nucleotide with a phosphorothioate (PS bond), a lower case “m” denotes that the nucleotide is modified with 2′-O-Me, and a lowercase “d” denotes that the nucleotide is a DNA nucleotide.

The five oligonucleotides were thawed and mixed together in equimolar quantities (about 30 μM each in a total volume of 2100 μL) and annealed at 85° C. for 3 minutes with shaking at 300 rpm, then the temperature was reduced to 25° C. with a 0.1° C./second ramp. The annealed oligos were mixed with 200 μL of T4 RNA ligase 2 (NEB, M0239B), 300 μL of a T4 DNA ligase buffer (NEB, cat. #B0202SVIAL), 20 μL of T4 DNA ligase (NEB, M0202M), 75 μL of a 40U/uL RNase inhibitor (Roche, cat. #9537589103), 305 μL of water (Corning, 46-000Cl) and incubated at 37° C. for 40 minutes with shaking at 300 rpm.

The mixture was purified by precipitation as follows. A volume of 11.25 mL of RNA precipitation buffer, prepared as described in Example 61, was mixed into the mixture for 5 minutes, then 41 mL of 100% ethanol (Fisher Scientific, BP2818-500) was added, and the mixture was stored at −20° C. for an hour. The mixture was then spun down at 4000×g for 30 minutes, and the supernatant was discarded. The pellet was washed with 3 mL of 70% ethanol pre-cooled at −20° C. and spun down at 4000×g for 5 minutes, and the supernatant was discarded. The ethanol wash was repeated once, and the resulting pellet was air dried for 10 minutes, before being resuspended in 1 mL of water.

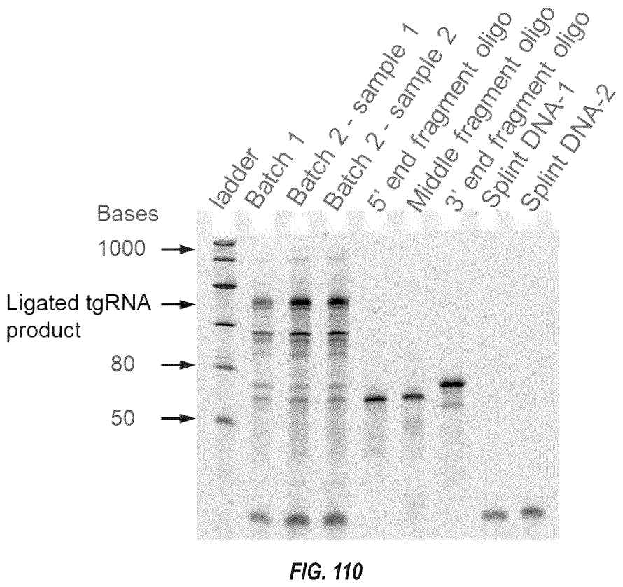

The procedure was repeated twice, resulting in two resuspended solution batches. One sample from the first batch and two samples from the second batch were assayed against samples from the five initial oligonucleotides on a 15% TBE-Urea gel (Invitrogen, EC6885BOX) using RNA loading buffer II (Invitrogen, AM8546G) and a low-range ssRNA ladder (NEB, N0364S). Ligation efficiency was determined based on the gel lines corresponding to the ligated tgRNA product, relative to the lines corresponding to traces of shorter oligonucleotides in each resuspended solution batch sample. The gel analysis is summarized for all three batches in Table 133, and shown in FIG. 110.

TABLE 133
Ligation efficiency from the gel analysis of the resuspended
solution following purification by precipitation.

| Start material | Ligation efficiency (%) |
|---|---|
| Batch 1 | 45 |
| Batch 2 - sample 1 | 48 |
| Batch 2 - sample 2 | 47 |

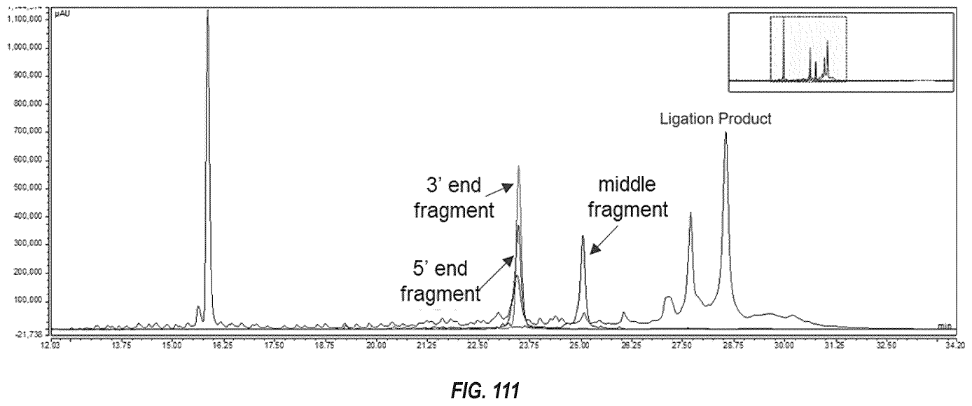

To confirm the production of the ligated tgRNA, and corroborate the results from the gel analysis, a sample from a resuspended solution obtained following the purification by precipitation (diluted to 5 μM) was analyzed using ultra-high-performance liquid chromatography (UPLC) on a Waters Acquity Peptide BEH C18 1.7 μM, 2.1×150 mm column coupled to an ultraviolet detector and a Thermo QExactive high-resolution mass spectrometer (UPLC-UV-HRMS), as follows. Mobile phase A comprised 150 mM 1,1,1,3,3,3-hexafluoroisopropanol (HFIP), 20 mM hexylamine (HA), and 5% methanol. Mobile phase B comprised of 75 mM HFIP, 10 mM HA, and 50% each of methanol and acetonitrile. Mobile phase C comprised methanol containing 0.2% formic acid. The column was operated at 70° C. with a flow rate of 200 uL/min, employing a gradient that comprised a first linear increase from 10% to 35% mobile phase B in 5.5 minutes in a background of mobile phase A (90%-65%), followed by a second linear increase to 55% mobile phase B/45% mobile phase A in 38 minutes. A 5-minute wash with 100% mobile phase B was then performed, followed by a 5-minute wash with 100% mobile phase C and a final column equilibration to 90% mobile phase B/10% mobile phase A over 10 minutes. The purity of the final ligated tgRNA product was determined as the fraction of total peak area (% AUC, also referred to as a % or area %) attributed to the corresponding signal in the UPLC-UV chromatogram. The UPLC-UV chromatogram for the resuspended solution batch is shown in FIG. 111. The measured purity of the final ligated tgRNA product was 37%.

Purification of the Crude Ligated 179-Nt tgRNA Using HPLC

Following the purification by precipitation as described above, the resuspended solution batch was further purified using preparative high-performance liquid chromatography (HPLC) on a Waters Acquity Peptide BEH C18 2.5 μM, 4.6×150 mm column coupled to an ultraviolet detector. Mobile phase A comprised 100 mM triethylamine acetate (TEAA), while mobile phase B comprised 100 mM TEAA and 40% acetonitrile. The column was operated at 75° C. with a flow rate of 500 μL/min, employing a gradient that comprised a linear increase from 10% to 30% mobile phase B in 55 minutes. A 5-minute wash with 100% mobile phase B was then performed, followed by a final column equilibration time of 10 minutes.

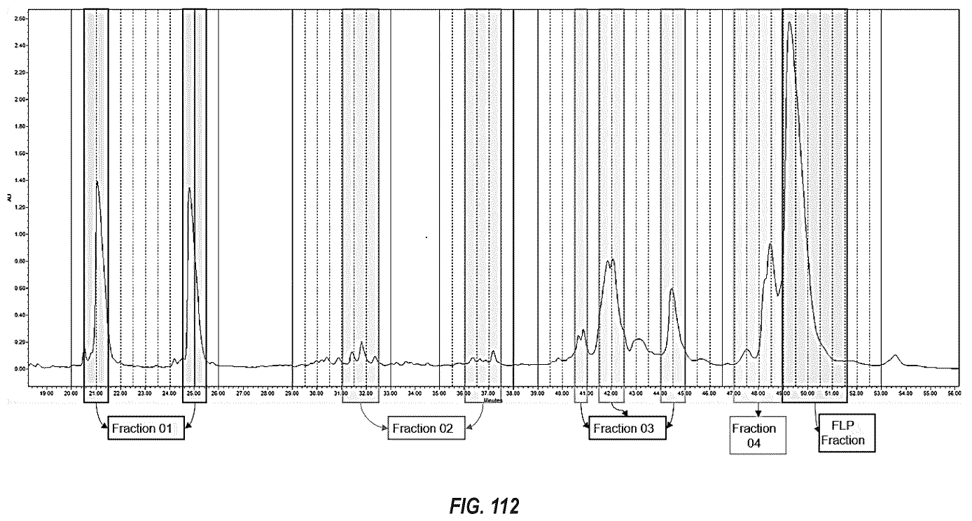

Fraction collection was enabled based on a retention time window. The ligated tgRNA, corresponding to the full-length product (FLP) fraction was subsequently concentrated and buffer-exchanged to water using a 15 mL-3 kDa cut-off Amicon filter. The fractionation and sample pools are shown in FIG. 112.

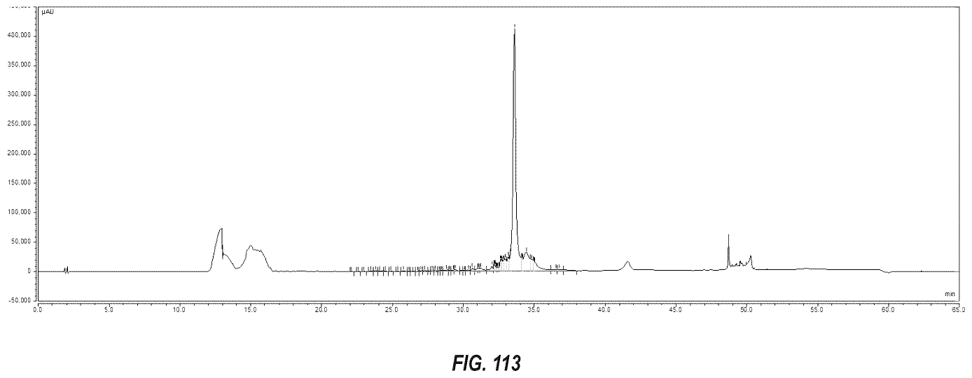

UPLC-UV was conducted to assess the purity of the ligated tgRNA product as described above. The chromatogram of the final product following HPLC purification is shown in FIG. 113. The final purity of the full-length product was determined to be 62%. The molecular weight of the final ligated tgRNA product was confirmed using the HRMS data.

### Example 63—Production of tgRNA Longer than 150 Nucleotides Through a One-Site Ligation Approach

In this Example, a production method was assessed for producing a template guide RNA (tgRNA) greater than 150 nucleotides in length by ligation of two oligonucleotides (with the aid of one splint oligonucleotides.

Production of a 179-Nt tgRNA Through Ligation of Two Oligonucleotides

A one-site guide ligation method was performed to produce a tgRNA with a length of 179 nucleotides at a 5 milligram scale, by ligating two split fragments of the desired final tgRNA product as shown in FIG. 106A and FIG. 106C.

The split fragment oligonucleotides listed in Table 134 were obtained from Genscript (Piscataway, NJ, USA). The splint DNA oligonucleotide listed in Table 134 was obtained from IDT (Coralville, IA, USA). The splint DNA oligonucleotide was used to ligate the two fragment oligonucleotides into the final full-length guide RNA product (G038241).

TABLE 134
Exemplary oligonucleotides used for the guide ligation procedure described herein.

| | | Molecular | Concentration | Purity (%) |
|---|---|---|---|---|
| | | Weight | in solution | measured by |
| Description | Sequence | (g/mol) | (uM) | UPLC |
| 5′end | mG*mA*mU*GUCUGCAGGCCAGAUG | 28093.79 | 100 | 31.2 |
| ligation split | AGUUUUAGAmGmCmUmAmGmAmAm | | | |
| fragment | AmUmAmGmCAAGUUAAAAUAAGGC | | | |
| | UAGUCCGUUAUCACGAAAGGGCACC | | | |
| | GAGUCG (SEQ ID NO: 466) | | | |
| 3′end | pGmUmGmCmCmAmCmAmGmUmGmU | 29294.94 | 100 | 20.1 |
| ligation split | mGmUmCmCmCmUmCmUdGdAdCdAd | | | |
| fragment | AdTdGdTdGdCdCdAdTdCdTdGdGdAdGd | | | |
| | CdCdCdTdCdAdAdTdGdAdTdCdCdTdGd | | | |
| | AdCdGdAdCdGdGdAdGdAdCdCdGdCdC | | | |
| | dGdTdCdGdTdCdGdAdCdAdAdGdCdCdT | | | |
| | CUGGCmCmUmG*mC (SEQ ID NO: 467) | | | |
| Splint DNA | CACACTGTGGCACCGACTCGGTGCCC | 8276.30 | 400 | Not available |
| | T (SEQ ID NO: 468) | | | |

Note:
*denotes that the nucleotide is linked to the next nucleotide with a phosphorothioate (PS bond), a lower case “m” denotes that the nucleotide is modified with 2′-O-Me, and a lowercase “d” denotes that the nucleotide is a DNA nucleotide.

The five oligonucleotides were thawed and mixed together in equimolar quantities (about 45 μM each in a total volume of 1350 μL) and annealed at 85° C. for 4 minutes with shaking at 300 rpm, then the temperature was reduced to 25° C. with a 0.1° C./second ramp. The annealed oligos were mixed with 200 μL of T4 RNA ligase 2 (NEB, M0239B), 300 μL of a 10×RNA ligase 2 buffer (NEB, cat. #B0239SVIAL), 75 μL of a 40U/uL RNase inhibitor (Roche, cat. #9537589103), 1075 μL of water (Corning, 46-000Cl) and incubated at 37° C. for 40 minutes with shaking at 300 rpm.

The mixture was purified by precipitation as follows. A volume of 11.25 mL of RNA precipitation buffer, prepared as described in Example 61, was mixed into the mixture for 5 minutes, then 41 mL of 100% ethanol (Fisher Scientific, BP2818-500) was added, and the mixture was stored at −20° C. for an hour. The mixture was then spun down at 4000×g for 30 minutes, and the supernatant was discarded. The pellet was washed with 3 mL of 70% ethanol pre-cooled at −20° C. and spun down at 4000×g for 5 minutes, and the supernatant was discarded. The ethanol wash was repeated once, and the resulting pellet was air dried for 10 minutes, before being resuspended in 1 mL of water.

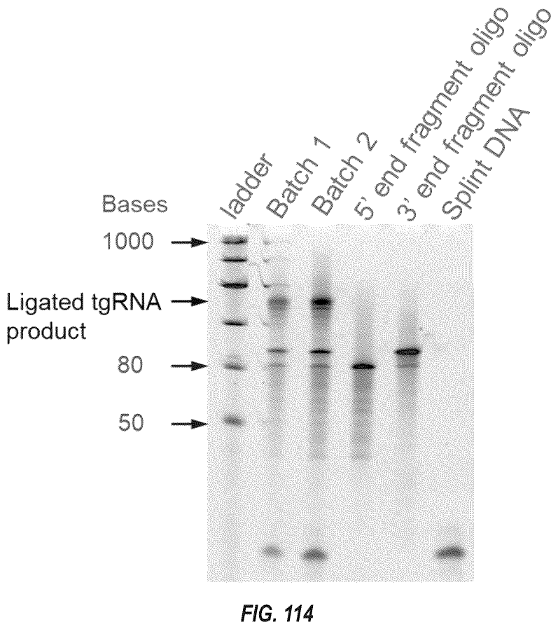

The procedure was repeated twice, resulting in two resuspended solution batches. One sample from each batch was assayed against samples from the three initial oligonucleotides on a 15% TBE-Urea gel (Invitrogen, EC6885BOX) using RNA loading buffer II (Invitrogen, AM8546G) and a low-range ssRNA ladder (NEB, N0364S). Ligation efficiency was determined based on the gel lines corresponding to the ligated tgRNA product, relative to the lines corresponding to traces of shorter oligonucleotides in each resuspended solution batch sample. The gel analysis is summarized in Table 135, and shown in FIG. 114.

TABLE 135
Ligation efficiency from the gel analysis of the resuspended
solution following purification by precipitation.

| Start material | Ligation efficiency (%) |
|---|---|
| Batch 1 | 65 |
| Batch 2 | 64 |

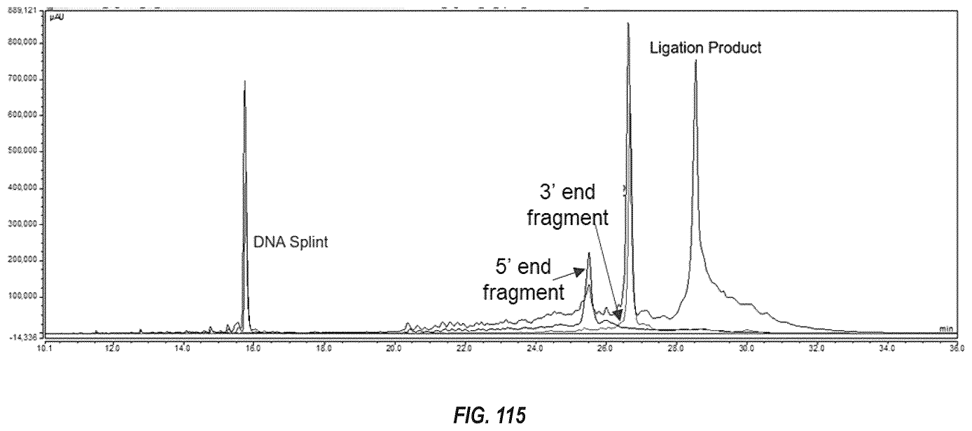

tgRNA production in a resuspended solution obtained after purification by precipitation was confirmed using UPLC-UV-HRMS, as described in Example 62. The UPLC-UV chromatogram is shown in FIG. 115. The measured purity of the ligated tgRNA product was 36%.

Purification of the Crude Ligated 179-Nt tgRNA Using HPLC

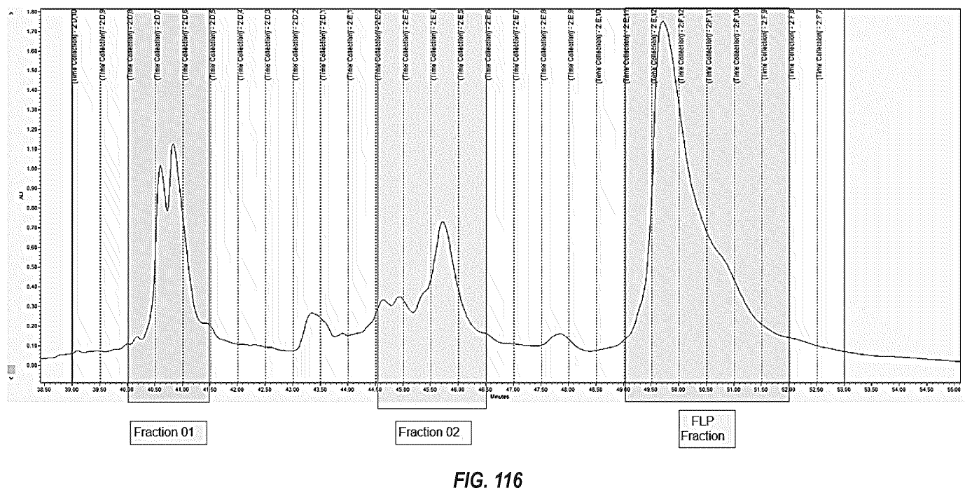

Following the purification by precipitation as described above, the resuspended solution batch was further purified using preparative high-performance liquid chromatography (HPLC) on a Waters Acquity Peptide BEH C18 2.5 μM, 4.6×150 mm column coupled to an ultraviolet detector, following the procedure described in Example 62. Fraction collection was enabled based on a retention time window as shown in FIG. 116. The ligated tgRNA (FLP) fraction was subsequently concentrated and buffer-exchanged to water using a 15 ml-3 kDa cut-off Amicon filter.

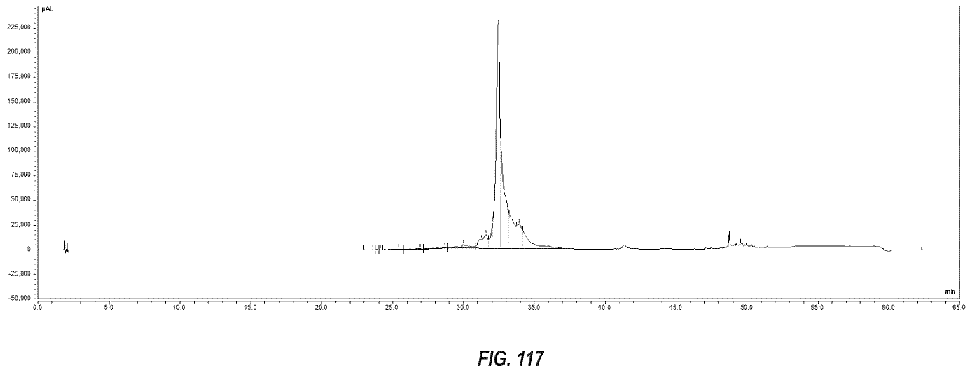

UPLC-UV-HRMS was conducted to assess the purity of the ligated tgRNA product as described above. The chromatogram of the final product following HPLC purification is shown in FIG. 117. The final purity of the full-length product was determined to be 43%. The molecular weight of the final ligated tgRNA product was confirmed using the HRMS data.

### Example 64—Exemplary Embodiments

Embodiment 1 is a system comprising a DNA-dependent DNA polymerase, a SpyCas9 nickase (nCas9), and a template guide RNA (tgRNA), wherein the tgRNA comprises a first polynucleotide and a second polynucleotide, wherein:
a) the first polynucleotide comprises a SpyCas9 guide RNA comprising, from 5′ to 3′:
   1) a spacer that is complementary to a target region of a target strand of a DNA duplex target nucleic acid; and
   2) a scaffold; and
b) the second polynucleotide is operably linked to the first polynucleotide and comprises, from 5′ to 3′:
   1) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; and
   2) a DNA-dependent DNA polymerase recruiting sequence (DRS) that is 6-20 nucleotides in length, optionally 10-14 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid, wherein the DRS comprises RNA nucleotides; and
   3) wherein the DRS comprises at least one of:
      1. a sequence not fully complementary to the DRS-complementary region of the non-target strand of the DNA duplex target nucleic acid;
      2. a 1-5 nucleotide, optionally a 1-3 nucleotide 3′ terminal tail comprising a 3′ terminal modified uridine nucleotide; or
      3. a 5′ terminal (D1) DNA nucleotide and a penultimate 5′ terminal (D2) RNA nucleotide wherein the 3′ end of the first polynucleotide is covalently linked to the 5′ end of the second polynucleotide by a phosphorothioate or phosphodiester linkage.

Embodiment 2 is the system of Embodiment 1, wherein the first polynucleotide is covalently linked to the second polynucleotide.

Embodiment 3 is the system of Embodiment 1 or 2, wherein the 3′ end of the first polynucleotide is covalently linked to the 5′ end of the second polynucleotide.

Embodiment 4 is the system of any one of Embodiments 1-3, wherein the first polynucleotide is linked to the second polynucleotide by a non-nucleotide linker.

Embodiment 5 is the system of any one of Embodiments 1-3, wherein the first polynucleotide is linked to the second polynucleotide by a phosphodiester bond or a phosphorothioate bond.

Embodiment 6 is the system of any one of Embodiments 1-5, wherein the 3′ terminal nucleotide of the template is a DNA nucleotide.

Embodiment 7 is the system of any one of Embodiments 1-6, wherein the 5′ terminal nucleotide of the DRS is a DNA nucleotide.

Embodiment 8 is the system of any one of Embodiments 1-7, wherein the penultimate 5′ terminal nucleotide of the DRS is an RNA nucleotide.

Embodiment 9 is the system of any one of Embodiments 1-8, wherein the 3′ terminal nucleotide of the template and the 5′ terminal nucleotide of the DRS are DNA nucleotides, and the penultimate 5′ terminal nucleotide of the DRS is an RNA nucleotide.

Embodiment 10 is the system of any one of Embodiments 1-6, wherein the 3′ terminal nucleotide in the DRS is an RNA nucleotide and the remaining nucleotides in the DRS are DNA nucleotides.

Embodiment 11 is the system of any one of Embodiments 1-10, wherein the template consists of DNA nucleotides.

Embodiment 12 is the system of any one of Embodiments 1-6, wherein the DRS consists of RNA nucleotides.

Embodiment 13 is the system of any one of Embodiments 1-12, wherein the sequence of the DRS that is complementary to the DRS-complementary region of the non-target strand of the DNA duplex target nucleic acid comprises nucleotides D2-D9 of the DRS, optionally D2-D8 or D3-D9 of the DRS.

Embodiment 14 is the system of any one of Embodiments 1-13, wherein the sequence of the DRS that is complementary to a DRS-complementary region of a non-target strand of the DNA duplex target nucleic acid comprises nucleotides D6-D13 of the DRS, optionally wherein 1, 2, 3, 4, 5, 6, 7, or 8 of nucleotides D6-D13 are DNA nucleotides.

Embodiment 15 is the system of Embodiment 1, wherein the tgRNA further comprises an affinity tag, wherein the first polynucleotide or the second polynucleotide are bound to a peptide or a peptide complex comprising the nCas9 and the DNA-dependent DNA polymerase by the affinity tag.

Embodiment 16 is the system of Embodiment 15, wherein 1-7 consecutive 5′ terminal nucleotides of the DRS are DNA nucleotides.

Embodiment 17 is the system of Embodiment 15 or 16, wherein the 5′ terminal and penultimate 5′ terminal nucleotides of the DRS are DNA nucleotides.

Embodiment 18 is the system of any one of Embodiments 15-17, wherein at least 3 consecutive 5′ terminal nucleotides of the DRS are DNA nucleotides.

Embodiment 19 is the system of any one of Embodiments 15-18, wherein 3 consecutive 5′ terminal nucleotides of the DRS are DNA nucleotides.

Embodiment 20 is the system of any one of Embodiments 15-19, wherein the affinity tag is located 5′ of the template sequence or the affinity tag is located 3′ of the DRS.

Embodiment 21 is the system of any one of Embodiments 15-20, wherein the tgRNA comprises two affinity tags.

Embodiment 22 is the system of any one of Embodiments 15-21, wherein the tgRNA comprises two affinity tags, further wherein both affinity tags are located 5′ of the template.

Embodiment 23 is the system of any one of Embodiments 15-22, wherein the tgRNA comprises two affinity tags, further wherein a first affinity tag is located 5′ of the template sequence and a second affinity tag is located 3′ of the DRS, wherein the DNA-dependent DNA polymerase is T5 DNA polymerase.

Embodiment 24 is the system of any one of Embodiments 15-23, wherein the affinity tag is an aptamer comprising one or more modified nucleotides.

Embodiment 25 is the system of any one of Embodiments 15-24, wherein the tgRNA comprises one affinity tag located 5′ of the template and has a 5′ terminus, and wherein the affinity tag is an aptamer comprising one or more modified nucleotide at the 5′ end thereof.

Embodiment 26 is the system of Embodiment 24 or 25, wherein the modified nucleotide is a 2′-O-methyl (2′-O-Me) modified nucleotide or a phosphorothioate (PS) linkage modified nucleotide.

Embodiment 27 is the system of any one of Embodiments 24-26, wherein the aptamer is located 5′ of the template sequence, further wherein the aptamer comprises a PS linkage between each of the first three nucleotides at the 5′ end.

Embodiment 28 is the system of Embodiment 26 or 27, wherein the aptamer is located 3′ of the DRS, further wherein the affinity tag comprises a PS linkage between the last two nucleotides at the 3′ end.

Embodiment 29 is the system of any one of Embodiments 24-27, wherein the aptamer is located 5′ of the template sequence, further wherein the aptamer comprises 2′-O-Me modified nucleotides at each of the first two nucleotides at the 5′ end.

Embodiment 30 is the system of any one of Embodiments 24-29, wherein the aptamer comprises a stem-loop, further wherein the aptamer comprises 2′-O-Me modified nucleotides at each nucleotide in the stem of the stem-loop.

Embodiment 31 is the system of any one of Embodiments 24-30, wherein the aptamer comprises 2′-O-Me modified nucleotides at each nucleotide.

Embodiment 32 is the system of any one of Embodiments 24-27 and 29-30, wherein the aptamer is located 5′ of the template sequence, further wherein the aptamer comprises 2′-O-Me modified nucleotides at each of the first two nucleotides at the 5′ end and a PS linkage between each of the first three nucleotides at the 5′ end.

Embodiment 33 is the system of any one of Embodiments 24-27, 29, 30 and 32, wherein the aptamer is located 5′ of the template sequence and comprises a stem-loop, further wherein the aptamer comprises 2′-O-Me modified nucleotides at each nucleotide in the stem of the stem-loop and a PS linkage between each of the first three nucleotides at the 5′ end.

Embodiment 34 is the system of any one of Embodiments 24, 26, 28, and 30, wherein the aptamer is located 3′ of the DRS and comprises a stem-loop, further wherein the aptamer comprises 2′-O-Me modified nucleotides at each nucleotide in the stem of the stem-loop and a PS linkage between the last two nucleotides at the 3′ end.

Embodiment 35 is the system of any one of Embodiments 1-34, wherein the DRS comprises a modified nucleotide.

Embodiment 36 is the system of any one of Embodiments 1-35, wherein the DRS comprises a modified nucleotide selected from:
a) 2′-O-methyl (2′-O-Me) modified nucleotide;
b) 2′-fluoro (2′-F) modified nucleotide;
c) an amine modified nucleotide;
d) an alkoxy modified nucleotide;
e) an LNA modified nucleotide;
f) methoxyethyl modified nucleotide;
g) a halo modified nucleotide; or
h) phosphorothioate (PS) linkage modified nucleotide;
optionally, wherein the modified nucleotide is a 2′-O-methyl (2′-O-Me) modified nucleotide or a phosphorothioate (PS) linkage modified nucleotide.

Embodiment 37 is the system of Embodiment 35 or 36, wherein the modification in the DRS comprises a phosphorothioate modification, and the DRS does not comprise a phosphorothioate modification at position D1-D9 in the DRS sequence.

Embodiment 38 is the system of any one of Embodiments 35-37, wherein the modification in the DRS comprises a phosphorothioate modification, and the DRS comprises a phosphorothioate modification at or 3′ of position D10 in the DRS sequence.

Embodiment 39 is the system of any one of Embodiments 35-38, wherein the modification in the DRS comprises a 2′-O-Me modification, and the DRS does not comprise a 2′-O-Me modification at position D1-D3 in the DRS sequence.

Embodiment 40 is the system of any one of Embodiments 35-39, wherein the modification in the DRS comprises a 2′-O-Me modification, and the DRS comprises a 2′-O-Me modification at or 3′ of position D4 in the DRS sequence.

Embodiment 41 is the system of any one of Embodiments 1-40, wherein the DNA-dependent DNA polymerase and nCas9 are provided as one or more polypeptides.

Embodiment 42 is the system of any one of Embodiments 1-40, wherein the DNA-dependent DNA polymerase and nCas9 are provided as one or more polynucleotides.

Embodiment 43 is the system of any one of Embodiments 1-42, wherein the DNA-dependent DNA polymerase comprises DNA polymerase K (polK), optionally human DNA polymerase K (polK), cynomolgus polK, or mouse polK, phage T5 DNA polymerase (T5 pol), DNA polymerase θ, E. coli DNA polymerase I, optionally wherein the E. coli dependent DNA polymerase is Klenow (exo-), or DNA polymerase N.

Embodiment 44 is the system of any one of Embodiments 1-43, wherein the DNA-dependent DNA polymerase is polK or T5 DNA polymerase.

Embodiment 45 is the system of Embodiments 1-44, comprising an nCas9-polymerase fusion polypeptide.

Embodiment 46 is the system of Embodiment 45, wherein the fusion polypeptide further comprises a heterologous nuclear localization signal (NLS).

Embodiment 47 is the system of Embodiment 45 or 46, wherein the fusion polypeptide comprises a linker or further comprises an additional linker.

Embodiment 48 is the system of Embodiment 45-47, wherein the polymerase is selected from: polymerase Θ, E. coli polymerase I, optionally an E. coli polymerase I Klenow (exo-), polymerase N, or Phi29 polymerase, wherein
a) the polymerase Θ and nCas9 are covalently linked sequentially in a polypeptide from N-terminus to C-terminus in an order selected from:
   4) nCas9-polymerase θ-NLS; or
   5) nCas9-linker-polymerase Θ-linker-NLS; or
b) the E. coli polymerase I and nCas9 are covalently linked sequentially in a polypeptide from N-terminus to C-terminus in an order selected from:
   6) nCas9-Klenow (exo-)-NLS;
   7) nCas9-linker-Klenow (exo-)-linker-NLS;
   8) NLS-nCas9-NLS-E coli PolI; or
   9) NLS-nCas9-linker-NLS-linker-E coli PolI; or
c) the polymerase N and nCas9 are covalently linked sequentially in a polypeptide from N-terminus to C-terminus in an order selected from:
   10) polymerase N-NLS-nCas9-NLS; or
   11) polymerase N-linker-NLS-nCas9-linker-NLS; or
d) the Phi29 polymerase and nCas9 are covalently linked sequentially in a polypeptide from N-terminus to C-terminus in an order selected from:
   12) NLS-nCas9-NLS-Phi29 polymerase;
   13) ii. NLS-nCas9-linker-NLS-linker-Phi29 polymerase;
   14) nCas9-Phi29 polymerase-NLS; or 15) nCas9-linker-Phi29 polymerase-linker-NLS.

Embodiment 49 is the system of Embodiment 46 or 47, wherein, the linker comprises an amino acid sequence of any one of SEQ ID NOs: 1596-1665, 1735, 1738, 1815, 1825, 1827, and 1829-1830.

Embodiment 50 is the system of any one of Embodiments 45-49, wherein the fusion polypeptide comprises an amino acid sequence that has at least 90%, 91%, 92%, 93%, 94%, 95%, 96%, 97%, 98%, 99%, or 100% identity to any one of SEQ ID NOs: 1024, 1027, 1030, 1033, 1036, 1039, 1042, 1045, 1048, 1053, 1056, 1059, 1074, 1081, 1084, 1086, 1094, 1097, 1100, 1102, 1112, 1114, 1124, 1129, 1134, 1137, 1142, 1147, 1152, 1157, 1162, 1167, 1172, 1192, 1195, 1198, 1219, 1224, 1229, 1234, 1236, 1239, 1254, and 1259, 1268, and 1273, 1276, 1300, 1303, 1307, 1373, 1376, 1388, 1447, 1450, 1453, 1923, 1926, 1929, 1932, 1935, and 1938, or a nucleic acid encoding the fusion protein sequence of any one of SEQ ID NOs: 1024, 1027, 1030, 1033, 1036, 1039, 1042, 1045, 1048, 1053, 1056, 1059, 1074, 1081, 1084, 1086, 1094, 1097, 1100, 1102, 1112, 1114, 1124, 1129, 1134, 1137, 1142, 1147, 1152, 1157, 1162, 1167, 1172, 1192, 1195, 1198, 1219, 1224, 1229, 1234, 1236, 1239, 1254, 1259, 1268, and 1273, 1276, 1300, 1303, 1307, 1373, 1376, 1388, 1477, 1450, 1453, 1923, 1926, 1929, 1932, 1935, and 1938.

Embodiment 51 is the system of any one of Embodiments 1-50, wherein the DNA-dependent DNA polymerase is a human DNA-dependent DNA polymerase.

Embodiment 52 is the system of any one of Embodiments 1-50, wherein the DNA-dependent DNA polymerase is a cynomolgus or mouse DNA-dependent DNA polymerase.

Embodiment 53 is the system of any one of Embodiments 1-52, wherein the DNA-dependent DNA polymerase is not a Phi29 DNA polymerase.

Embodiment 54 is the system of any one of Embodiments 1-53, wherein the DNA-dependent DNA polymerase does not comprise at least 8 point mutations.

Embodiment 55 is the system of any one of Embodiments 1-54, wherein the DNA-dependent DNA polymerase does not comprise at least 5 point mutations.

Embodiment 56 is the system of any one of Embodiments 1-55, wherein the wild-type DNA-dependent DNA polymerase has a polymerization rate at least twice as high, optionally at least five times as high at 37° C. as compared to 30° C.

Embodiment 57 is the system of any one of Embodiments 1-56, comprising a DNA polymerase K (polK), comprising an amino acid sequence that has at least 90%, 91%, 92%, 93%, 94%, 95%, 96%, 97%, 98%, 99%, or 100% identity to amino acid residues 19-526 of SEQ ID NO: 1021; or an amino acid sequence that has at least 90%, 91%, 92%, 93%, 94%, 95%, 96%, 97%, 98%, 99%, or 100% identity to SEQ ID NO: 1021 or 1206, optionally wherein the polk includes or lacks an N-terminal methionine relative to SEQ ID NO: 1021 or 1206; or a nucleic acid encoding the polk, comprising a nucleotide sequence that has at least 90%, 91%, 92%, 93%, 94%, 95%, 96%, 97%, 98%, 99%, or 100% identity to SEQ ID NO: 1020 or 1205, optionally wherein the nucleotide sequence includes or lacks a start or stop codon relative to SEQ ID NO: 1020 or 1205.

Embodiment 58 is the system of any one of Embodiments 1-56, comprising a T5 polymerase (T5 pol) comprising an amino acid sequence that has at least 90%, 91%, 92%, 93%, 94%, 95%, 96%, 97%, 98%, 99%, or 100% identity to any one of SEQ ID NO: 1116 or 1121, optionally wherein the T5 pol includes or lacks an N-terminal methionine relative to SEQ ID NO: 1116 or 1121; or a nucleic acid encoding the T5, comprising a nucleotide sequence that has at least 90%, 91%, 92%, 93%, 94%, 95%, 96%, 97%, 98%, 99%, or 100% identity to SEQ ID NO: 1115 or 1120, optionally wherein the nucleotide sequence includes or lacks a start or stop codon relative to SEQ ID NO: 1115 or 1120.

Embodiment 59 is the system of any one of Embodiments 1-58, wherein the DNA-dependent DNA polymerase comprises a heterodimerization domain or further comprises an additional heterodimerization domain.

Embodiment 60 is the system of any one of Embodiments 1-59, wherein the DNA-dependent DNA polymerase and nCas9 are covalently linked sequentially in a polypeptide from N-terminus to C-terminus in an order of NLS-nCas9-linker-NLS-linker-DNA-dependent DNA polymerase-NLS-NLS-linker-heterodimerization domain.

Embodiment 61 is the system of any one of Embodiments 1-60, wherein the DNA-dependent DNA polymerase and nCas9 are covalently linked sequentially in a polypeptide from N-terminus to C-terminus in an order, wherein the order is NLS-nCas9-linker-NLS-linker-DNA-dependent DNA polymerase-linker-NLS-linker-heterodimerization domain.

Embodiment 62 is the system of any one of Embodiments 59-61, wherein the heterodimerization domain comprises a coiled coil heterodimerization domain.

Embodiment 63 is the system of any one of Embodiments 59-62, wherein the heterodimerization domain comprises an EV, EI, KV, or KI domain.

Embodiment 64 is the system of any one of Embodiments 59-63, wherein the heterodimerization domain comprises an EI or KI domain.

Embodiment 65 is the system of any one of Embodiments 59-64, wherein the heterodimerization domain comprises SEQ ID NO: 1589, SEQ ID NO: 1593, or SEQ ID NO: 1595.

Embodiment 66 is the system of any one of Embodiments 1-44, 51-59, and 62-65, comprising:
a) a first polypeptide comprising the DNA-dependent DNA polymerase operably linked to a first split-intein, or a nucleic acid encoding the first polypeptide; and (ii) a second polypeptide comprising the nCas9 operably linked to a second split-intein that is complementary to the first split intein, or a nucleic acid encoding the second polypeptide;
b) a first polypeptide comprising a first portion of the DNA-dependent DNA polymerase operably linked to a first split-intein, or a nucleic acid encoding the first polypeptide; and (ii) a second polypeptide comprising a second portion of the DNA-dependent DNA polymerase and an nCas9 operably linked to a second split-intein that is complementary to the first split-intein; or
c) a first polypeptide comprising a DNA-dependent DNA polymerase and a first portion of an nCas9 operably linked to a first split-intein, or a nucleic acid encoding the first polypeptide; and (ii) a second polypeptide comprising a second portion of the nCas9 operably linked to a second split-intein that is complementary to the first split-intein, or a nucleic acid encoding the second polypeptide.

Embodiment 67 is the system of Embodiment 66, wherein the first split-intein and the second split-intein are capable of binding to each other to undergo intein-mediated protein splicing.

Embodiment 68 is the system of Embodiment 66 or 67, wherein the first split-intein is an N-split-intein and the second split-intein is a C-split-intein.

Embodiment 69 is the system of Embodiment 66 or 67, wherein the first split-intein is a C-split-intein and the second split intein is an N-split-intein.

Embodiment 70 is the system of any one of Embodiments 66-69, wherein the first split-intein and second split-intein are complementary inteins selected from Cfa, M86, Npu, NpuSsp, gp41-1, gp41-8, NrdJ-1, IMPDH-1, SspDnaX, SspGyrB, Cth-Ter, NrdA-2, Mja-KlbA, RBS1, TE3S11, and Rma.

Embodiment 71 is the system of any one of Embodiments 66-70, wherein the first split-intein and second split-intein are Cfa-inteins.

Embodiment 72 is the system of Embodiment 71, wherein the first split-intein is a CfaC split-intein and the second split-intein is a CfaN split-intein.

Embodiment 73 is the system of Embodiment 72, wherein the CfaC split-intein comprises an amino acid sequence having at least 90% identity to SEQ ID NO: 1746 and the CfaN split-intein comprises an amino acid sequence having at least 90% identity to SEQ ID NO: 1744.

Embodiment 74 is the system of Embodiment 73, wherein the CfaC split-intein comprises the amino acid sequence of SEQ ID NO: 1746 and the CfaN split-intein comprises the amino acid sequence of SEQ ID NO: 1744.

Embodiment 75 is the system of any one of Embodiments 66-74, wherein the DNA-dependent DNA polymerase or the first portion of the DNA-dependent DNA polymerase is operably linked to the first split-intein by a first peptide linker.

Embodiment 76 is the system of Embodiment 75, wherein the first peptide linker comprises 30 amino acids, optionally wherein the first peptide linker comprises an amino acids sequence at least 90%, 95%, 98%, or 100% identical to SEQ ID NO: 1829.

Embodiment 77 is the system of any one of Embodiments 66-76, wherein the nCas9 or second portion of the nCas9 is operably linked to the second split-intein by a second peptide linker.

Embodiment 78 is the system of Embodiment 77, wherein the second peptide linker is SGGS (SEQ ID NO: 1738).

Embodiment 79 is the system of any one of Embodiments 66-78, wherein the DNA-dependent DNA polymerase or the first portion of the DNA-dependent DNA polymerase is operably linked to a heterologous nuclear localization signal (NLS).

Embodiment 80 is the system of any one of Embodiments 66-79, wherein the nCas9 or the second portion of the nCas9 is operably linked to a heterologous nuclear localization signal (NLS).

Embodiment 81 is the system of Embodiment 79 or 80, wherein the heterologous NLS is selected from an SV40 NLS, SV40-c-Myc, and nucleoplasmin.

Embodiment 82 is the system of any one of Embodiments 1-81, further comprising at least one chromatin remodeler.

Embodiment 83 is the system of Embodiment 82, wherein the DNA-dependent DNA polymerase, nCas9, and chromatin remodeler are covalently linked sequentially in a polypeptide from N-terminus to C-terminus in an order selected from:
a) chromatin remodeler-nCas9-DNA-dependent DNA polymerase;
b) nCas9-chromatin remodeler-DNA-dependent DNA polymerase; or
c) nCas9-DNA-dependent DNA polymerase-chromatin remodeler.

Embodiment 84 is the system of Embodiment 83, further comprising a nuclear localization signal (NLS).

Embodiment 85 is the system of Embodiment 84, wherein the DNA-dependent DNA polymerase, nCas9, and chromatin remodeler are covalently linked sequentially in a polypeptide from N-terminus to C-terminus in an order selected from:
a) NLS-chromatin remodeler-nCas9-DNA-dependent DNA polymerase-NLS;
b) NLS-chromatin remodeler-nCas9-NLS-DNA-dependent DNA polymerase-NLS;
c) NLS-nCas9-chromatin remodeler-DNA-dependent DNA polymerase-NLS; or
d) NLS-nCas9-chromatin remodeler-NLS-DNA-dependent DNA polymerase-NLS; nCas9-DNA-dependent DNA polymerase-chromatin remodeler-NLS.

Embodiment 86 is the system of any one of Embodiments 82-85, further comprising one or more linkers between the NLS, nCas9, DNA-dependent DNA polymerase, and the chromatin remodeler.

Embodiment 87 is the system of any one of Embodiments 82-86, wherein the at least one chromatin remodeler is selected from a polypeptide comprising HMGB1, HMGN1, HMGN2, HMGN3a, HMGN3b, HMGN4, HMGN5, histone H1.0, histone H1.2, CHD1, ISWI, DNA helicase, TOP1, herpesvirus 8 LANA, CMV IE1 or a domain thereof.

Embodiment 88 is the system of any one of Embodiments 1-87, wherein the at least one chromatin remodeler comprises an HMGB1 polypeptide.

Embodiment 89 is the system of Embodiment 88, wherein the HMGB1 polypeptide comprises a sequence at least 90%, 91%, 92%, 93%, 94%, 95%, 96%, 97%, 98%, 99%, or 100% identical to SEQ ID NO: 1783.

Embodiment 90 is the system of Embodiment 88 or 89, wherein the HMGB1 polypeptide comprises an HMGB1 Box B domain and at least 7 contiguous amino acid residues C-terminal to the HMGB1 Box B domain, and wherein the HMGB1 polypeptide lacks all or part of an acidic tail domain.

Embodiment 91 is the system of any one of Embodiments 88-90, wherein the HMGB1 polypeptide comprises a cryptic nuclear localization signal (NLS).

Embodiment 92 is the system of Embodiment 91, wherein the cryptic NLS comprises the sequence of EKSKKKK (SEQ ID NO: 1787).

Embodiment 93 is the system of any one of Embodiments 88-92, wherein the HMGB1 polypeptide comprises a receptor for advanced glycation end-products (RAGE) binding domain.

Embodiment 94 is the system of any one of Embodiments 93, wherein the RAGE binding domain comprises the sequence of SEQ ID NO: 1803.

Embodiment 95 is the system of any one of Embodiments 88-94, wherein the HMGB1 polypeptide comprises 7-20 amino acid residues C-terminal to the HMGB1 Box B domain.

Embodiment 96 is the system of any one of Embodiments 88-95, wherein the HMGB1 polypeptide comprises the sequence of SEQ ID NO: 1804.

Embodiment 97 is the system of any one of Embodiments 88-96, wherein the HMGB1 polypeptide lacks a sequence at least 50%, 60%, 70%, 80%, 90%, 94%, or 97% identical to SEQ ID NO: 1788 or lacks the sequence of SEQ ID NO: 1788.

Embodiment 98 is the system of any one of Embodiments 88-97, wherein the HMGB1 polypeptide lacks transcriptional stimulatory function.

Embodiment 99 is the system of any one of Embodiments 88-98, wherein the HMGB1 polypeptide comprises a cryptic NLS that is located C-terminal to the HMGB1 Box B domain.

Embodiment 100 is the system of any one of Embodiments 88-99, wherein the at least 7-20 contiguous amino acid residues C-terminal end of the HMGB1 Box B domain comprise the cryptic NLS.

Embodiment 101 is the system of any one of Embodiments 88-100, wherein the HMGB1 polypeptide comprises a cryptic NLS that is located C-terminal to the HMGB1 Box B domain.

Embodiment 102 is the system of any one of Embodiments 88-101, wherein the cryptic NLS immediately follows the C-terminal end of the HMGB1 Box B domain.

Embodiment 103 is the system of any one of Embodiments 88-102, wherein the HMGB1 polypeptide lacks an HMGB1 Box A domain.

Embodiment 104 is the system of any one of Embodiments 88-103, wherein the HMGB1 polypeptide comprises two HMGB1 Box B domains.

Embodiment 105 is the system of any one of Embodiments 88-104, wherein the cryptic NLS is located C-terminal to the C-terminal end of the two HMGB1 Box B domains.

Embodiment 106 is the system of any one of Embodiments 88-102 and 104-105, wherein the HMGB1 polypeptide comprises an HMGB1 Box A domain.

Embodiment 107 is the system of any one of Embodiments 88-102 and 104-106, wherein the Box B domain is located C-terminal to the HMGB1 Box A domain.

Embodiment 108 is the system of any one of Embodiments 88-102 and 104-107, wherein the HMGB1 polypeptide comprises, from N-terminus to C-terminus:
a) HMGB1 Box A domain-HMGB1 Box B domain-cryptic NLS;
b) HMGB1 Box B domain-HMGB1 Box B domain-cryptic NLS; or
c) HMGB1 Box B domain-cryptic NLS.

Embodiment 109 is the system of any one of Embodiments 88-108, wherein the HMGB1 polypeptide comprises at least 90% sequence identity to SEQ ID NO: 1783-1792 and 1803-1804.

Embodiment 110 is the system of any one of Embodiments 88-109, wherein the HMGB1 polypeptide comprises the amino acid sequence of SEQ ID NO: 1783-1792 and 1803-1804.

Embodiment 111 is the system of any one of Embodiments 66-110, wherein the first or second polypeptide further comprises an HMGB1 polypeptide.

Embodiment 112 is the system of Embodiment 111, wherein:
a) the first polypeptide comprises, from N-terminus to C-terminus: HMGB-nCas9-N-split-intein; and
b) the second polypeptide comprises, from N-terminus to C-terminus: C-split-intein-DNA-dependent DNA polymerase.

Embodiment 113 is the system of Embodiment 111 or 112, wherein:
a) the first polypeptide comprises, from N-terminus to C-terminus: NLS-HMGB-linker-nCas9-linker-N-split-intein; and
b) the second polypeptide comprises, from N-terminus to C-terminus:
   1)C-split-intein-linker-NLS-DNA-dependent DNA polymerase-linker-coiled coil heterodimerization domain; or
2)C-split-intein-linker-NLS-DNA-dependent DNA polymerase-linker-NLS;
3) optionally wherein the second polypeptide comprises at least one enhancer of template-based genome editing at the C-terminus.

Embodiment 114 is A template guide RNA (tgRNA), wherein the tgRNA comprises a first polynucleotide and a second polynucleotide, wherein:
c) the first polynucleotide comprises a SpyCas9 guide RNA, comprising, from 5′ to 3′,
   1) a spacer that is complementary to a target region of a target strand of a DNA duplex target nucleic acid; and
   2) a scaffold;
d) the second polynucleotide is operably linked to the first polynucleotide and comprises, from 5′ to 3′:
   1) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; and
   2) ii) a DNA-dependent DNA polymerase recruiting sequence (DRS) having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid, optionally wherein the DRS is 6-20 nucleotides in length, further optionally 10-14 nucleotides in length.

Embodiment 115 is the tgRNA of Embodiment 114, wherein the DRS comprises at least one of:
1) a sequence not fully complementary to the DRS-complementary region of the non-target strand of the DNA duplex target nucleic acid;
2) a 1-5 nucleotide, optionally a 1-3 nucleotide 3′ terminal tail comprising a 3′ terminal modified uridine nucleotide; or
3) a 5′ terminal (D1) DNA nucleotide and a penultimate 5′ terminal (D2) RNA nucleotide wherein the 3′ end of the first polynucleotide is covalently linked to the 5′ end of the second polynucleotide by a phosphorothioate or phosphodiester linkage.

Embodiment 116 is the tgRNA of Embodiment 114 or 115, wherein the spacer sequence is 20 nucleotides in length.

Embodiment 117 is the tgRNA of any one of Embodiments 114-116, wherein the spacer of the guide RNA comprises a modified nucleotide.

Embodiment 118 is the tgRNA of any one of Embodiments 114-117, wherein the spacer of the guide RNA comprises a phosphorothioate (PS) modification, a 2′-O-methyl (2′-O-Me) modified nucleotide, or a 2′-F modified nucleotide.

Embodiment 119 is the tgRNA of any one of Embodiments 114-118, wherein the spacer of the guide RNA comprises DNA nucleotides.

Embodiment 120 is the tgRNA of any one of Embodiments 114-119, wherein the spacer of the guide RNA comprises a DNA nucleotide at at least 2, 3, 4, 5, 6, 7, 8, 9, or 10 nucleotides.

Embodiment 121 is the tgRNA of any one of Embodiments 114-120, wherein the spacer of the guide RNA comprises a DNA nucleotide at 1, 2, 3, 4, 5, 6, 7, 8, 9, 10, or 11 nucleotides.

Embodiment 122 is the tgRNA of any one of Embodiments 114-121, wherein the spacer of the guide RNA comprises a DNA nucleotide at one or more, two or more, three or more, or four or more of positions S1, S2, S3, S4, S5, S9, S10, S11, S12, S13, S17, S18, or S20, optionally wherein the spacer of the guide RNA comprises a DNA nucleotide at one or more of positions S11, S12, and S18, optionally at each of positions S11, S12, and S18.

Embodiment 123 is the tgRNA of any one of Embodiments 114-118, wherein the spacer of the guide RNA does not comprise DNA nucleotides.

Embodiment 124 is the tgRNA of any one of Embodiments 114-119, wherein the spacer of the guide RNA comprises a modified nucleotide at one or more of the first five nucleotides at the 5′ end.

Embodiment 125 is the tgRNA of any one of Embodiments 114-120, wherein the spacer of the guide RNA comprises a modified nucleotide at one or more of the first three nucleotides at the 5′ end of the spacer.

Embodiment 126 is the tgRNA of any one of Embodiments 114-125, wherein the spacer of the guide RNA does not comprise a modification at one or more of positions S6, S7, S8, S15, S16, and S19.

Embodiment 127 is the tgRNA of any one of Embodiments 114-126, wherein the spacer of the guide RNA does not comprise a modification at one or more of positions S4, S5, S6, S7, S8, S11, S12, S15, S16, S18, and S19.

Embodiment 128 is the tgRNA of any one of Embodiments 114-127, wherein the spacer of the guide RNA comprises a modification at one or more of positions S4, S5, S9, S10, S11, S12, S13, S14, S17, S18, and S20.

Embodiment 129 is the tgRNA of any one of Embodiments 114-128, wherein the spacer of the guide RNA comprises 3 PS linkages between the 4 consecutive 5′ terminal nucleotides of the spacer.

Embodiment 130 is the tgRNA of any one of Embodiments 114-129, wherein the spacer of the guide RNA comprises a PS linkage between positions S7 and S8.

Embodiment 131 is the tgRNA of any one of Embodiments 114-130, wherein the spacer of the guide RNA comprises a 2′-O-Me modified nucleotide or a 2′-F modified nucleotide in at least 2, 3, 4, 5, 6, 7, 8, 9, or 10 nucleotides.

Embodiment 132 is the tgRNA of any one of Embodiments 114-131, wherein the spacer of the guide RNA comprises 2′-O-Me modified nucleotides at each of the first three nucleotides (S1, S2, and S3) at the 5′ end of the spacer.

Embodiment 133 is the tgRNA of any one of Embodiments 114-132, wherein the spacer of the guide RNA comprises 2′-F modified nucleotides at one or more of positions S9, S10, S11, S13, S14, S17, and S18.

Embodiment 134 is the tgRNA of Embodiment 133, wherein the spacer of the guide RNA comprises a 2′-O-Me modified nucleotide at position S4 and 2′-F modified nucleotides at each of positions S9, S10, S11, S13, S14, S17, and S18

Embodiment 135 is the tgRNA of Embodiment 133, wherein the spacer of the guide RNA comprises a 2′-O-Me modified nucleotide at position S4, 2′-F modified nucleotides at each of positions S9, S10, S11, S13, S14, S17, and S18, and a PS linkage between positions S7 and S8.

Embodiment 136 is the tgRNA of any one of Embodiments 114-132, wherein the spacer of the guide RNA comprises 2′-O-Me modified nucleotides at one or more of positions S4, S5, S9, S10, S11, S12, S13, S14, S17, S18, and S20.

Embodiment 137 is the tgRNA of Embodiment 136, wherein the spacer of the guide RNA comprises 2′-O-Me modified nucleotides at one or more of positions S4, S5, S9, S10, S13, S14, S17 and S20.

Embodiment 138 is the tgRNA of Embodiment 137, wherein the spacer of the guide RNA comprises 2′-O-Me modified nucleotides at each of positions S9, S10, S13, S14, S17, and S20.

Embodiment 139 is the tgRNA of Embodiment 137, wherein the spacer of the guide RNA comprises 2′-O-Me modified nucleotides at each of positions S9, S10, S17, and S20 and DNA nucleotides at each of positions S11, S12, and S18.

Embodiment 140 is the tgRNA of Embodiment 136, wherein:
a) the spacer of the guide RNA comprises 2′-O-Me modified nucleotides at each of S1, S2, S3, S9, S10, S13, S14, S17, and S20; and
b) the spacer of the guide RNA does not comprise a modification at each of S4, S5, S6, S7, S8, S11, S12, S15, S16, S18, and S19.

Embodiment 141 is the tgRNA of Embodiment 136, wherein:
a) the spacer of the guide RNA comprises 2′-O-Me modified nucleotides at each of S1, S2, S3, S9, S10, S13, S14, S17, and S20;
b) the spacer of the guide RNA comprises DNA nucleotides at each of positions S11, S12, and S18; and
c) the spacer of the guide RNA does not comprise a modification at each of S4, S5, S6, S7, S8, S15, S16, and S19.

Embodiment 142 is the tgRNA of any one of Embodiments 114-141, wherein the scaffold of the guide RNA comprises the nucleotide sequence of SEQ ID NO: 27 or 40.

Embodiment 143 is the tgRNA of Embodiment 142, wherein:
a) nucleotides 5′-GUUUUAGAGCUAGAAAUAGCAAGUUAAAAU-3′ or 5′-GUUUUAGACGUAGAAAUACGAAGUUAAAAU-3′ constitute a repeat/antirepeat (R/AR) region, wherein nucleotides 1-6 (LS1-LS6) and 25-30 (LS7-LS12) of the R/AR constitute a lower stem (LS) region, nucleotides 7-8 (B1-B2) and 21-24 (B3-B6) of the R/AR constitute a bulge region; and nucleotides 9-20 (US1-US12) of the R/AR constitute an upper stem (US) region;
b) nucleotides 5′-AAGGCUAGUCCGUUAUCA-3′ constitute a nexus region (N1-N18);
c) nucleotides 5′-CGAAAG-3′ constitute a hairpin 1 (H1) region (from 5′ to 3′, H1-2, H1-5 to H1-8, and H1-11);
d) nucleotides 5′-GCACCGAGUCGGUGC-3′ constitute a hairpin 2 (H2) region H2-1 to H2-15); and
e) a G nucleotide between H1 region and H2 region constitutes nucleotide n;
wherein the scaffold comprises at least one region with unmodified and modified nucleotides selected from:
   1) the lower stem region comprising modified nucleotides at LS1, LS8, LS10, and LS12; nucleotides not modified with 2′-O-Me at LS2, LS3, LS4, LS5;
   2) the bulge region comprising nucleotides not modified with 2′-O-Me at B5 and B6;
   3) the upper stem region comprising modified nucleotides at at least 10 positions selected from US1, US2, US3, US4, US5, US6, US7, US8, US9, US10, US11, and US12, optionally at all positions US1, US2, US3, US4, US5, US6, US7, US8, US9, US10, US11, and US12;
   4) the nexus region comprising modified nucleotides at N1, N2, N4, N7, N11, N12, and N17; and nucleotides not modified with 2′-O-Me at N6, N8, N9, N10, N13, and N14;
   5) the hairpin 1 (H1) region, comprising modified nucleotides at H1-2, H1-6, H1-7, H1-8, and H1-11; and nucleotides not modified with 2′-O-Me at H1-5;
   6) the hairpin 2 (H2) region comprising modified nucleotides at H2-1, H2-2, H2-3, H2-4, H2-5, H2-6, H2-7, H2-12, H2-13, H2-14, and H2-15; and nucleotides not modified with 2′-O-Me at H2-8, H2-9, and H2-10; and
   7) the n is a modified or unmodified nucleotide.

Embodiment 144 is the tgRNA of Embodiment 143, wherein the scaffold comprises at least one region with unmodified and modified nucleotides selected from:
a) the lower stem region comprising modified nucleotides at LS1, LS8, LS10, and LS12; nucleotides not modified with 2′-O-Me at LS2, LS3, LS4, LS5, and LS6;
b) the bulge region comprising nucleotides not modified with 2′-O-Me at B5 and B6;
c) the upper stem region comprising modified nucleotides at at least 10 positions selected from US1, US2, US3, US4, US5, US6, US7, US8, US9, US10, US11, and US12, optionally at all positions US1, US2, US3, US4, US5, US6, US7, US8, US9, US10, US11, and US12;
d) the nexus region comprising modified nucleotides at N1, N2, N4, N7, N11, N12, and N17; and nucleotides not modified with 2′-O-Me at N6, N8, N9, N10, N13, N14, and N16;
e) the hairpin 1 (H1) region, comprising modified nucleotides at H1-2, H1-6, H1-7, H1-8, and H1-11; and nucleotides not modified with 2′-O-Me at H1-5;
f) the hairpin 2 (H2) region comprising modified nucleotides at H2-1, H2-2, H2-3, H2-4, H2-5, H2-6, H2-7, H2-11, H2-12, H2-13, H2-14, and H2-15; and nucleotides not modified with 2′-O-Me at H2-8, H2-9, and H2-10; and
g) the n is a modified or unmodified nucleotide.

Embodiment 145 is the tgRNA of Embodiment 142 or 143, wherein the scaffold comprises at least one region with unmodified and modified nucleotides selected from:
a) the lower stem region comprising modified nucleotides at LS1, LS8, LS10, and LS12; nucleotides not modified with 2′-O-Me at LS2, LS3, LS4, LS5, LS6, and LS9;
b) the bulge region comprising nucleotides not modified with 2′-O-Me at B5 and B6;
c) the upper stem region comprising modified nucleotides at at least 10 positions selected from US1, US2, US3, US4, US5, US6, US7, US8, US9, US10, US11, and US12, optionally at all positions US1, US2, US3, US4, US5, US6, US7, US8, US9, US10, US11, and US12;
d) the nexus region comprising modified nucleotides at N1, N2, N4, N7, N11, N12, N16, and N17; and nucleotides not modified with 2′-O-Me at N6, N8, N9, N10, N13, N14, and N16;
e) the hairpin 1 (H1) region, comprising modified nucleotides at H1-2, H1-6, H1-7, H1-8, and H1-11; and nucleotides not modified with 2′-O-Me at H1-5;
f) the hairpin 2 (H2) region comprising modified nucleotides at H2-1, H2-2, H2-3, H2-4, H2-5, H2-6, H2-7, H2-11, H2-12, H2-13, H2-14, and H2-15; and nucleotides not modified with 2′-O-Me at H2-8, H2-9, and H2-10; and
g) the n is a modified or unmodified nucleotide.

Embodiment 146 is the tgRNA of any one of Embodiments 142-145, wherein the lower stem region comprises modified nucleotides at LS1, LS8, LS10, and LS12; nucleotides not modified with 2′-O-Me at LS2, LS3, LS4, LS5, LS6, LS7, LS9, and LS11.

Embodiment 147 is the tgRNA of any one of Embodiments 142-146, wherein the lower stem region comprises unmodified nucleotides at LS2, LS3, LS4, and LS5, optionally at LS2, LS3, LS4, LS5, LS6, LS7, LS9, and LS11.

Embodiment 148 is the tgRNA of any one of Embodiments 142-147, wherein the lower stem region comprises unmodified nucleotides at LS7, LS9, and LS11.

Embodiment 149 is the tgRNA of any one of Embodiments 142-148, wherein the bulge region comprises modified nucleotides at B2 and B3.

Embodiment 150 is the tgRNA of any one of Embodiments 142-149, wherein the bulge region comprises unmodified nucleotides at B5 and B6, optionally at B1, B4, B5, and B6.

Embodiment 151 is the tgRNA of any one of Embodiments 142-150, wherein the modified nucleotides of the upper stem are 2′-O-Me modified nucleotides.

Embodiment 152 is the tgRNA of any one of Embodiments 142-151, wherein the nexus region comprises unmodified nucleotides at N6, N8, N9, N10, N13, N14, and N18, optionally at N3, N5, N6, N8, N9, N10, N13, N14, N15, and N18.

Embodiment 153 is the tgRNA of any one of Embodiments 142-152, wherein the nexus region comprises unmodified nucleotides at N3, N5, N6, N8, N9, N10, N13, N14, N15, and N18.

Embodiment 154 is the tgRNA of any one of Embodiments 142-153, wherein the hairpin 1 region comprises an unmodified nucleotide at H1-5.

Embodiment 155 is the tgRNA of any one of Embodiments 142-154, wherein the hairpin 2 region comprises unmodified nucleotides at H2-8, H2-9, and H2-10.

Embodiment 156 is the tgRNA of any one of Embodiments 142-155, further comprising a 3′ tail.

Embodiment 157 is the tgRNA of any one of Embodiments 142-156, wherein the gRNA comprises a 3′ end modification.

Embodiment 158 is the tgRNA of any one of Embodiments 114-157, wherein the scaffold sequence comprises an internal linker.

Embodiment 159 is the tgRNA of any one of Embodiments 114-158, wherein the scaffold sequence comprises (i) an internal linker in or substitutes for the upper stem region; (ii) an internal linker in a nexus region; or (iii) an internal linker in the hairpin 1 region.

Embodiment 160 is the tgRNA of any one of Embodiments 114-159, wherein the guide RNA comprises
a) the sequence of mGUUUUAGmAmGmC(L1)mGmCmAAGUUmAAmAAmUmAmAGmGCUmAG UCmCmGUUAUmCAmC(L1)mGGmGmCmAmCmCmGmAGUCmGmGmUmGm C (SEQ ID NO: 272) or a sequence having at least 90%, 95%, or 98% identity to SEQ ID NO: 272;
b) the sequence of GUUUUAGAmGmCmUmA(L1)mUmAmGmCAAGUUAAAAUAAGGCUAGUCC GUUAUCAC(L1)GGGCACCGAGUCGGmUmGmC (SEQ ID NO: 211) or a sequence having at least 90%, 95%, or 98% identity to SEQ ID NO: 211;
c) the sequence of GUUUUAGAmGmC(L1)mGmCAAGUUAAAAUAAGGCUAGUCCGUUAUCAC(L1)GGGCACCGAGUCGGmUmGmC (SEQ ID NO: 232) or a sequence having at least 90%, 95%, or 98% identity to SEQ ID NO: 232;
d) the sequence of mGUUUUAGmAmGmCmUmA(L1)mUmAmGmCmAAGUUmAAmAAmUmAmA GmGCUmAGUCmCmGUUAUmCAmC(L1)mGGmGmCmAmCmCmGmAGUCm GmGmUmGmC (SEQ ID NO: 224) or a sequence having at least 90%, 95%, or 98% identity to SEQ ID NO: 224,
wherein L1 denotes that the linker is S18, and L3 denotes that the linker is S6.

Embodiment 161 is the tgRNA of any one of Embodiments 114-160, wherein the spacer or the scaffold of the guide RNA comprises a modified nucleotide.

Embodiment 162 is the tgRNA of any one of Embodiments 114-161, wherein the first polynucleotide comprises a SpyCas9 guide RNA comprising, from 5′ to 3′, a guide sequence and any one of SEQ ID NOs: 5, 27, 35, 210, 269, 310, 322-325, 326, 347-349, 370, 201-208, 371, 373-374, and 384-400, 438.

Embodiment 163 is the tgRNA of any one of Embodiments 114-161, wherein the first polynucleotide is covalently linked to the second polynucleotide.

Embodiment 164 is the tgRNA of any one of Embodiments 114-162, wherein the 3′ end of the first polynucleotide is covalently linked to the 5′ end of the second polynucleotide.

Embodiment 165 is the tgRNA of any one of Embodiments 114-163, wherein the first polynucleotide is linked to the second polynucleotide by a non-nucleotide linker, optionally by a phosphodiester bond or a phosphorothioate bond.

Embodiment 166 is the tgRNA of any one of Embodiments 114-165, wherein the template comprises a mismatch to the genomic sequence, optionally 1-4 mismatches to the genomic sequence.

Embodiment 167 is the tgRNA of any one of Embodiments 114-166, wherein the mismatch that directs a template-based edit in the genomic DNA is within nucleotides T1-T25, optionally within nucleotides T1-T15 of the template.

Embodiment 168 is the tgRNA of any one of Embodiments 114-167, wherein the template-based edit in the genomic DNA comprises a substitution.

Embodiment 169 is the tgRNA of any one of Embodiments 114-168, wherein the template-based edit in the genomic DNA comprises an insertion of a nucleotide in the genomic sequence.

Embodiment 170 is the tgRNA of any one of Embodiments 114-169, wherein the template-based edit in the genomic DNA comprises a deletion of a nucleotide in the genomic sequence.

Embodiment 171 is the tgRNA of any one of Embodiments 114-170, wherein the template-based edit in the genomic sequence results in a change in an amino acid sequence encoded by a locus in which the genomic sequence is present.

Embodiment 172 is the tgRNA of any one of Embodiments 114-171, wherein the template-based edit in the genomic sequence does not result in a change in an amino acid sequence encoded by a locus in which the genomic sequence is present.

Embodiment 173 is the tgRNA of any one of Embodiments 114-172, wherein the template comprises DNA nucleotides.

Embodiment 174 is the tgRNA of any one of Embodiments 114-173, wherein the 3′ terminal nucleotide of the template is a DNA nucleotide.

Embodiment 175 is the tgRNA of any one of Embodiments 114-174, wherein the template consists of DNA nucleotides from nucleotide 4 (T4) from the 3′ end of the template to the 5′ end of the template.

Embodiment 176 is the tgRNA of any one of Embodiments 114-175, wherein the template consists of DNA nucleotides.

Embodiment 177 is the tgRNA of any one of Embodiments 114-176, wherein the template comprises RNA nucleotides.

Embodiment 178 is the tgRNA of any one of Embodiments 114-177 and 178, wherein the template comprises 0-1 RNA nucleotides, optionally 1 RNA nucleotide, at the 3′ end of the template.

Embodiment 179 is the tgRNA of any one of Embodiments 114-177 and 178-179, wherein the template comprises 1-3 RNA nucleotides, optionally only 1 RNA nucleotide, at the 3′ end of the template.

Embodiment 180 is the tgRNA of any one of Embodiments 114-179, wherein the DRS is 6-18 nucleotides in length, optionally 8-18 or 10-12 nucleotides in length.

Embodiment 181 is the tgRNA of any one of Embodiments 114-179, wherein the DRS is 10-16 or 12-14 nucleotides in length.

Embodiment 182 is the tgRNA of any one of Embodiments 114-180, wherein the sequence of the DRS that is complementary to the DRS-complementary region of the non-target strand of the DNA duplex target nucleic acid comprises nucleotides 5-10 5′ of the PAM in the non-target strand of the DNA duplex target nucleic acid.

Embodiment 183 is the tgRNA of any one of Embodiments 114-181, wherein the DRS comprises a sequence that is complementary to at least 6 nucleotides of the DRS-complementary region of the non-target strand of the DNA duplex target nucleic acid comprising nucleotides D2-D9 of the DRS, optionally D2-D8 or D3-D9 of the DRS.

Embodiment 184 is the tgRNA of any one of Embodiments 114-182, wherein the DRS comprises a sequence that is complementary to a DRS-complementary region of a non-target strand of the DNA duplex target nucleic acid comprising nucleotides D6-D13 of the DRS, optionally wherein 1, 2, 3, 4, 5, 6, 7, or 8 of nucleotides D6-D13 are DNA nucleotides.

Embodiment 185 is the tgRNA of any one of Embodiments 114-184, wherein the DRS comprises a sequence that is not fully complementary to the DRS-complementary region of the non-target strand of the DNA duplex target nuclei acid.

Embodiment 186 is the tgRNA of any one of Embodiments 114-185, wherein the DRS comprises RNA nucleotides.

Embodiment 187 is the tgRNA of any one of Embodiments 114-186, wherein the penultimate 5′ terminal nucleotide of the DRS (D2) is an RNA nucleotide.

Embodiment 188 is the tgRNA of any one of Embodiments 114-187, wherein each nucleotide from the penultimate 5′ nucleotide (D2) to the 3′ end nucleotide in the DRS is an RNA nucleotide.

Embodiment 189 is the tgRNA of any one of Embodiments 114-188, wherein the 3′ terminal nucleotide of the DRS is an RNA nucleotide.

Embodiment 190 is the tgRNA of any one of Embodiments 114-189, wherein the DRS consists of RNA nucleotides.

Embodiment 191 is the tgRNA of any one of Embodiments 114-189, wherein the DRS comprises DNA nucleotides.

Embodiment 192 is the tgRNA of any one of Embodiments 114-189 and 191, wherein the 5′ terminal nucleotide of the DRS (D1) is a DNA nucleotide.

Embodiment 193 is the tgRNA of any one of Embodiments 114-189 and 191-192, wherein the DRS comprises a 3′ end and a 5′ end, wherein 0-7 nucleotides at the 5′ end of the DRS (nucleotides D1-D7) are DNA nucleotides.

Embodiment 194 is the tgRNA of any one of Embodiment 193 wherein one or more of nucleotides 1-7 nucleotides at the 5′ end of the DRS (nucleotides D1-D7) are DNA nucleotides.

Embodiment 195 is the tgRNA of Embodiment 193, wherein one or more of nucleotides 1-5 at the 5′ end of the DRS (nucleotides D1-D5) are DNA nucleotides, optionally wherein the remaining nucleotides in the DRS are RNA nucleotides.

Embodiment 196 is the tgRNA of any one of Embodiment 193, wherein one or more of nucleotides 1-3 at the 5′ end of the DRS (nucleotides D1-D3), optionally the 5′ terminal nucleotide in the DRS (nucleotide D1), is a DNA nucleotide.

Embodiment 197 is the tgRNA of Embodiment 193, wherein nucleotides 1 and 3 at the 5′ end of the DRS (nucleotides D1 and D3) are DNA nucleotides, optionally wherein the remaining nucleotides in the DRS are RNA nucleotides.

Embodiment 198 is the tgRNA of Embodiment 193, wherein nucleotides 1, 3, and 5 at the 5′ end of the DRS (nucleotides D1, D3, and D5) are DNA nucleotides, optionally wherein the remaining nucleotides in the DRS are RNA nucleotides.

Embodiment 199 is the tgRNA of any one of Embodiments 114-189, and 191-198, wherein the 3′ terminal nucleotide of the template and the 5′ terminal nucleotide of the DRS are DNA nucleotides, and the penultimate 5′ terminal nucleotide of the DRS is an RNA nucleotide.

Embodiment 200 is the tgRNA of any one of Embodiments 114-189 and 191-199, wherein the 3′ terminal nucleotide in the DRS is an RNA nucleotide and the remaining nucleotides in the DRS are DNA nucleotides.

Embodiment 201 is the tgRNA of any one of Embodiments 114-189 and 191-199, wherein the DRS consists of DNA nucleotides.

Embodiment 202 is the tgRNA of Embodiment 200 or 201, wherein the spacer of the guide RNA comprises 2′-O-Me modified nucleotides at one or more of positions S1, S2, S3, S4, S5, S9, S10, S11, S12, S13, S14, S17, S18, and S20.

Embodiment 203 is the tgRNA of Embodiment 200 or 201, wherein the spacer of the guide RNA does not comprise a modification at one or more of positions S6, S7, S8, S15, S16, and S19.

Embodiment 204 is the tgRNA of any one of Embodiments 200-202, wherein:
a) the spacer of the guide RNA comprises 2′-O-Me modified nucleotides at each of S1, S2, S3, S9, S10, S13, S14, S17, and S20; and
b) the spacer of the guide RNA does not comprise a modification at each of S4, S5, S6, S7, S8, S11, S12, S15, S16, S18, and S19.

Embodiment 205 is the tgRNA of any one of Embodiments 197-199, wherein:
a) the spacer of the guide RNA comprises 2′-O-Me modified nucleotides at each of S1, S2, S3, S9, S10, S13, S14, S17, and S20;
b) the spacer of the guide RNA comprises DNA nucleotides at each of positions S11, S12, and S18; and
c) the spacer of the guide RNA does not comprise a modification at each of S4, S5, S6, S7, S8, S15, S16, and S19.

Embodiment 206 is the tgRNA of any one of Embodiments 114-205, wherein the DRS comprises a 3′ tail at the 3′ end of the DRS, wherein the tail is 1-5 nucleotides, optionally 1-3 nucleotides in length.

Embodiment 207 is the tgRNA of Embodiment 206, wherein the 3′ tail of the DRS comprises an RNA nucleotide.

Embodiment 208 is the tgRNA of Embodiment 206 or 207, wherein the 3′ tail comprises a uridine nucleotide, optionally wherein the 3′ terminal nucleotide is a modified uridine nucleotide.

Embodiment 209 is the tgRNA of any one of Embodiments 114-208, wherein the DRS comprises a modified nucleotide.

Embodiment 210 is the tgRNA of any one of Embodiments 114-209, wherein the DRS comprises a modified nucleotide selected from:
a) 2′-O-methyl (2′-O-Me) modified nucleotide;
b) 2′-fluoro (2′-F) modified nucleotide;
c) an amine modified nucleotide;
d) an alkoxy modified nucleotide;
e) an LNA modified nucleotide;
f) methoxyethyl modified nucleotide;
g) a halo modified nucleotide; or
h) phosphorothioate (PS) linkage modified nucleotide;
optionally, wherein the modified nucleotide is a 2′-O-methyl (2′-O-Me) modified nucleotide or a phosphorothioate (PS) linkage modified nucleotide.

Embodiment 211 is the tgRNA of Embodiment 209 or 210, wherein the modification in the DRS comprises a phosphorothioate modification, and the DRS does not comprise a phosphorothioate modification at position D1-D9 in the DRS sequence.

Embodiment 212 is the tgRNA of any one of Embodiments 209-211, wherein the modification in the DRS comprises a phosphorothioate modification, and the DRS comprises a phosphorothioate modification at or 3′ of position D10 in the DRS sequence.

Embodiment 213 is the tgRNA of any one of Embodiments 209-212, wherein the modification in the DRS comprises a 2′-O-Me modification, and the DRS does not comprise a 2′-O-Me modification at position D1-D3 in the DRS sequence.

Embodiment 214 is the tgRNA of any one of Embodiments 209-213, wherein the modification in the DRS comprises a 2′-O-Me modification, and the DRS does not comprise a 2′-O-Me modification at the 5′ terminal and 3′ terminal nucleotides of the DRS.

Embodiment 215 is the tgRNA of any one of Embodiments 209-214, wherein the modification in the DRS comprises a 2′-O-Me modification, and the DRS comprises a 2′-O-Me modification at or 3′ of position D4 in the DRS sequence.

Embodiment 216 is the tgRNA of any one of Embodiments 209-215, wherein the modification in the DRS comprises a 2′-O-Me or a 2′-F modification, and the DRS comprises a 2′-O-Me or 2′-F modification at the 3′ terminal nucleotide in the DRS.

Embodiment 217 is the tgRNA of any one of Embodiments 209-216, wherein the DRS comprises PS linkages between 2, 3, 4, 5, or 6 consecutive 3′ terminal nucleotides of the DRS

Embodiment 218 is the tgRNA of any one of Embodiments 209-217, wherein the DRS comprises a PS linkage between the 3′ terminal and penultimate 3′ terminal nucleotides of the DRS.

Embodiment 219 is the tgRNA of any one of Embodiments 209-218, wherein the DRS comprises 3 PS linkages between the 4 consecutive 3′ terminal nucleotides of the DRS.

Embodiment 220 is the tgRNA of any one of Embodiments 209-219, wherein the DRS comprises a PS linkage between 2 nucleotides other than the 3′ terminal and penultimate 3′ terminal nucleotides of the DRS.

Embodiment 221 is the tgRNA of any one of Embodiments 209-220, wherein the DRS comprises a phosphorothioate modification, and wherein the DRS does not comprise a phosphorothioate modification at position D1-D9 in the DRS sequence.

Embodiment 222 is the tgRNA of any one of Embodiments 209-221, wherein the DRS comprises a phosphorothioate modification at or 3′ of position D10 in the DRS sequence.

Embodiment 223 is the tgRNA of any one of Embodiments 114-161, and 166-222, wherein the first polynucleotide is not covalently linked to the second polynucleotide.

Embodiment 224 is the tgRNA of any one of Embodiments 114-161, 166-189, and 191-223, wherein 1-7 consecutive 5′ terminal nucleotides of the DRS are DNA nucleotides.

Embodiment 225 is the tgRNA of any one of Embodiments 114-161, 166-186, and 191-196, and 198-224, wherein the 5′ terminal and penultimate 5′ terminal nucleotides of the DRS are DNA nucleotides.

Embodiment 226 is the tgRNA of any one of Embodiments 114-161, 166-186, and 191-225, wherein at least 3 consecutive 5′ terminal nucleotides of the DRS are DNA nucleotides.

Embodiment 227 is the tgRNA of any one of Embodiments 114-161, 166-186, and 191-226, wherein 3 consecutive 5′ terminal nucleotides of the DRS are DNA nucleotides

Embodiment 228 is the tgRNA of any one of Embodiments 114-227, wherein the tgRNA further comprises an affinity tag, wherein the first polynucleotide or the second polynucleotide are bound to a peptide or a peptide complex comprising an nCas9 and a DNA-dependent DNA polymerase by the affinity tag.

Embodiment 229 is the tgRNA of any one of Embodiments 114-161, 166-189, and 191-228, wherein 1-7 consecutive 5′ terminal nucleotides of the DRS are DNA nucleotides.

Embodiment 230 is the tgRNA of any one of Embodiments 114-161, 166-186, and 191-196, and 198-229, wherein the 5′ terminal and penultimate 5′ terminal nucleotides of the DRS are DNA nucleotides.

Embodiment 231 is the tgRNA of any one of Embodiments 114-161, 166-186, and 191-196, and 198-230, wherein at least 3 consecutive 5′ terminal nucleotides of the DRS are DNA nucleotides.

Embodiment 232 is the tgRNA of any one of Embodiments 114-161, 166-186, and 191-196, and 198-231, wherein 3 consecutive 5′ terminal nucleotides of the DRS are DNA nucleotides.

Embodiment 233 is the tgRNA of any one of Embodiments 228-232, wherein the affinity tag is located 5′ of the template sequence or the affinity tag is located 3′ of the DRS.

Embodiment 234 is the tgRNA of any one of Embodiments 228-233, wherein the tgRNA comprises two affinity tags.

Embodiment 235 is the tgRNA of any one of Embodiments 228-234, wherein the tgRNA comprises two affinity tags, further wherein both affinity tags are located 5′ of the template.

Embodiment 236 is the tgRNA of any one of Embodiments 228-235, wherein the tgRNA comprises two affinity tags, further wherein a first affinity tag is located 5′ of the template sequence and a second affinity tag is located 3′ of the DRS, wherein the DNA-dependent DNA polymerase is T5 DNA polymerase.

Embodiment 237 is the tgRNA of any one of Embodiments 228-236, wherein the affinity tag is an aptamer comprising one or more modified nucleotides.

Embodiment 238 is the tgRNA of any one of Embodiments 228-237, wherein the tgRNA comprises one affinity tag located 5′ of the template and has a 5′ terminus, and wherein the affinity tag is an aptamer comprising one or more modified nucleotide at the 5′ end thereof.

Embodiment 239 is the tgRNA of Embodiment 237 or 238, wherein the modified nucleotide is a 2′-O-methyl (2′-O-Me) modified nucleotide or a phosphorothioate (PS) linkage modified nucleotide.

Embodiment 240 is the tgRNA of any one of Embodiments 237-239, wherein the aptamer is located 5′ of the template sequence, further wherein the aptamer comprises a PS linkage between each of the first three nucleotides at the 5′ end.

Embodiment 241 is the tgRNA of any one of Embodiments 237-240, wherein the aptamer is located 3′ of the DRS, further wherein the affinity tag comprises a PS linkage between the last two nucleotides at the 3′ end.

Embodiment 242 is the tgRNA of any one of Embodiments 237-241, wherein the aptamer is located 5′ of the template sequence, further wherein the aptamer comprises 2′-O-Me modified nucleotides at each of the first two nucleotides at the 5′ end.

Embodiment 243 is the tgRNA of any one of Embodiments 237-242, wherein the aptamer comprises a stem-loop, further wherein the aptamer comprises 2′-O-Me modified nucleotides at each nucleotide in the stem of the stem-loop.

Embodiment 244 is the tgRNA of any one of Embodiments 237-243, wherein the aptamer comprises 2′-O-Me modified nucleotides at each nucleotide.

Embodiment 245 is the tgRNA of any one of Embodiments 237-240 and 242-244, wherein the aptamer is located 5′ of the template sequence, further wherein the aptamer comprises 2′-O-Me modified nucleotides at each of the first two nucleotides at the 5′ end and a PS linkage between each of the first three nucleotides at the 5′ end.

Embodiment 246 is the tgRNA of any one of Embodiments 237-240 and 242-245, wherein the aptamer is located 5′ of the template sequence and comprises a stem-loop, further wherein the aptamer comprises 2′-O-Me modified nucleotides at each nucleotide in the stem of the stem-loop and a PS linkage between each of the first three nucleotides at the 5′ end.

Embodiment 247 is the tgRNA of any one of Embodiments 236, 241, and 243-244, wherein the aptamer is located 3′ of the DRS and comprises a stem-loop, further wherein the aptamer comprises 2′-O-Me modified nucleotides at each nucleotide in the stem of the stem-loop and a PS linkage between the last two nucleotides at the 3′ end.

Embodiment 248 is A system comprising:
a) a first polynucleotide encoding a first polypeptide comprising a DNA-dependent DNA polymerase, or portion thereof, operably linked to a first split-intein; and
b) a second polynucleotide encoding a second polypeptide comprising a SpyCas9 nickase (nCas9), or portion thereof, operably linked to a second split-intein that is complementary to the first split-intein, wherein the first split-intein and the second split-intein undergo trans-splicing reaction, resulting in the formation of a single fusion polypeptide comprising from N-terminus to C-terminus, the DNA-dependent DNA polymerase and the nCas9.

Embodiment 249 is the system of Embodiment 248, wherein:
a) (i) a first polynucleotide encoding the DNA-dependent DNA polymerase operably linked to the first split-intein; and (ii) the second polynucleotide encoding the nCas9 operably linked to the second split-intein that is complementary to the first split-intein;
b) (i) the first polynucleotide encoding a first portion of the DNA-dependent DNA polymerase operably linked to the first split-intein; and (ii) the second polynucleotide encoding a second portion of the DNA-dependent DNA polymerase and the nCas9 operably linked to the second split-intein that is complementary to the first split-intein; or
c) (i) the first polynucleotide encoding the DNA-dependent DNA polymerase and a first portion of the nCas9 operably linked to the first split-intein; and (ii) the second polynucleotide encoding a second portion of the nCas9 operably linked to the second split-intein that is complementary to the first split-intein.

Embodiment 250 is the system of Embodiment 248 or 249, wherein:
a) the DNA-dependent DNA polymerase is a polK comprising an amino acid sequence that has at least 90%, 91%, 92%, 93%, 94%, 95%, 96%, 97%, 98%, 99%, or 100% identity to amino acid residues 19-526 of SEQ ID NO: 1021; or an amino acid sequence that has at least 90%, 91%, 92%, 93%, 94%, 95%, 96%, 97%, 98%, 99%, or 100% identity to SEQ ID NO: 1021 or 1206, optionally wherein the polk includes or lacks an N-terminal methionine relative to SEQ ID NO: 1021 or 1206; or
b) the DNA-dependent DNA polymerase is a T5 pol comprising an amino acid sequence that has at least 90%, 91%, 92%, 93%, 94%, 95%, 96%, 97%, 98%, 99%, or 100% identity to any one of SEQ ID NO: 1116 or 1121, optionally wherein the T5 pol includes or lacks an N-terminal methionine relative to SEQ ID NO: 1116 or 1121.

Embodiment 251 is the system of any one of Embodiments 248-250, wherein the first split-intein and the second split-intein are capable of binding to each other to undergo intein-mediated protein splicing.

Embodiment 252 is the system of any one of Embodiments 248-251, wherein the first split-intein is an N-split-intein and the second split-intein is a C-split-intein.

Embodiment 253 is the system of any one of Embodiments 248-252, wherein the first split-intein is a C-split-intein and the second split-intein is an N-split-intein.

Embodiment 254 is the system of any one of Embodiments 248-253, wherein the first split-intein and second split-intein are complementary inteins selected from Cfa, M86, Npu, NpuSsp, gp41-1, gp41-8, NrdJ-1, IMPDH-1, SspDnaX, SspGyrB, Cth-Ter, NrdA-2, Mja-KlbA, RBS1, TE3S11, and Rma.

Embodiment 255 is the system of any one of Embodiments 248-254, wherein the first split-intein and second split-intein are Cfa-inteins.

Embodiment 256 is the system of Embodiment 255, wherein the first split-intein is a Cfa C-split-intein and the second split-intein is a Cfa N-split-intein.

Embodiment 257 is the system of Embodiment 256, wherein the Cfa-C split-intein comprises an amino acid sequence having at least 90% identity to SEQ ID NO: 1746 and the Cfa-N split-intein comprises an amino acid sequence having at least 90% identity to SEQ ID NO: 1744.

Embodiment 258 is the system of Embodiment 255 or 256, wherein the Cfa C-split-intein comprises the amino acid sequence of SEQ ID NO: 1746 and the Cfa N-split-intein comprises the amino acid sequence of SEQ ID NO: 1744.

Embodiment 259 is the system of any one of Embodiments 248-258, wherein the first polynucleotide further encodes a first peptide linker that operably links the first split-intein to the DNA-dependent DNA polymerase.

Embodiment 260 is the system of Embodiment 259, wherein the first peptide linker comprises 30 amino acids.

Embodiment 261 is the system of Embodiment 259 or 260, wherein the first peptide linker comprises an amino acid sequence at least 90%, 95%, 98%, or 100% identical to SEQ ID NO: 1829.

Embodiment 262 is the system of any one of Embodiments 248-261, wherein the second polynucleotide further encodes a second peptide linker that operably links the second split-intein to nCas9.

Embodiment 263 is the system of Embodiment 262, wherein the second peptide linker is SGGS (SEQ ID NO: 1738).

Embodiment 264 is the system of any one of Embodiments 248-263, wherein the first polynucleotide further encodes a heterologous nuclear localization signal (NLS) that is operably linked to the DNA-dependent DNA polymerase.

Embodiment 265 is the system of any one of Embodiments 248-264, wherein the second polynucleotide further encodes a heterologous nuclear localization signal (NLS) that is operably linked to the nCas9.

Embodiment 266 is the system of Embodiment 264 or 265, wherein the heterologous nuclear localization signal is selected from an SV40 NLS, c-Myc NLS, and nucleoplasmin NLS.

Embodiment 267 is the system of any one of Embodiments 248-266, wherein the first polynucleotide or second polynucleotide further comprise a 5′ cap selected from Cap0, Cap1, and Cap2.

Embodiment 268 is the system of Embodiment 267, wherein the 5′ cap is a cap analog.

Embodiment 269 is the system of any one of Embodiments 248-268, further comprising at least one enhancer of template-based genome editing selected from Vpx and FEN-1.

Embodiment 270 is the system of any one of Embodiments 248-268, further comprising at least two enhancers of template-based genome editing comprising Vpx and FEN-1.

Embodiment 271 is the system of Embodiment 269 or 270, wherein Vpx or FEN1 are encoded on the first polynucleotide and operably linked to the DNA-dependent DNA polymerase.

Embodiment 272 is the system of Embodiment 269 or 270, wherein Vpx or FEN1 are encoded on the second polynucleotide and operably linked to the nCas9.

Embodiment 273 is the system of Embodiment 269, further comprising a third polynucleotide encoding at least one enhancer of template-based genome editing selected from Vpx and FEN-1.

Embodiment 274 is the system of Embodiment 270, further comprising a third polynucleotide encoding at least two enhancers of template-based genome editing comprising Vpx and FEN-1.

Embodiment 275 is the system of any one of Embodiments 248-274, wherein the first polynucleotide encodes a fusion protein comprising the first split-intein, the DNA-dependent DNA polymerase, one or more peptide linkers, and one or more NLSs.

Embodiment 276 is the system of Embodiment 275, wherein the first polynucleotide encodes a first polypeptide comprising sequentially from N-terminus to C-terminus the first split-intein, the DNA-dependent DNA polymerase, and an NLS.

Embodiment 277 is the system of Embodiment 276, wherein the first polypeptide further comprises a peptide linker between the first split-intein and the DNA-dependent DNA polymerase.

Embodiment 278 is the system of Embodiment 277, wherein the first polypeptide further comprises a peptide linker between the NLS and the DNA-dependent DNA polymerase.

Embodiment 279 is the system of any one of Embodiments 248-278, wherein the second polynucleotide encodes a second polypeptide comprising one or more NLSs, the nCas9, one or more peptide linkers, and the second split-intein.

Embodiment 280 is the system of Embodiment 279, wherein the second polypeptide comprises sequentially from N-terminus to C-terminus an NLS, the nCas9, and the second split-intein.

Embodiment 281 is the system of Embodiment 280, wherein the second polypeptide further comprises a peptide linker between the nCas9 and the second split-intein.

Embodiment 282 is the system of any one of Embodiments 248-281, wherein:
a) the first polynucleotide comprises an open reading frame (ORF) of at least 90%, 91%, 92%, 93%, 94%, 95%, 96%, 97%, 98%, 99%, or 100% identical to any one of SEQ ID NOs: 1278, 1281, 1363, 1366, and 1378; 1391, 1395, 1399, 1403, 1407, 1411, 1415, 1419, 1423, 1427, 1431, 1435, 1439, 1443, 1907, 1913, 1940, 1943 or a sequence at least 90%, 91%, 92%, 93%, 94%, 95%, 96%, 97%, 98%, 99%, or 100% identical to any one of SEQ ID NOs: 1277, 1280, 1362, 1365, 1377, 1906, 1912, 1939, 1942, 3006-3008, 3010, 3012, and 3017-3018; or
b) the second polynucleotide comprises an open reading frame (ORF) of at least 90%, 91%, 92%, 93%, 94%, 95%, 96%, 97%, 98%, 99%, or 100% identical to any one of SEQ ID NOs: 1284, 1287, 1290, 1293, 1296, 1369, 1381, 1384, 1389, 1393; 1397, 1401, 1405, 1409, 1413, 1417, 1421, 1425, 1429, 1433, 1437, 1441, 1901, 1904, 1910, 1916, 1918, 1946, 1949, 1952, 1955, 1958 or a sequence at least 90%, 91%, 92%, 93%, 94%, 95%, 96%, 97%, 98%, 99%, or 100% identical to any one of SEQ ID NOs: 1283, 1286, 1289, 1292, 1295, 1368, 1380, 1383, 1900, 1903, 1909, 1915, 1919, 1945, 1948, 1951, 1954, 1957, 3001-3005, 3009, 3011, 3013-3016, and 3019-3022.

Embodiment 283 is a system comprising:
a) a DNA-dependent DNA polymerase and a SpyCas9 nickase (nCas9); and
b) at least one chromatin remodeler operably linked to the nCas9 and the DNA-dependent DNA polymerase.

Embodiment 284 is the system of Embodiment 283, wherein the DNA-dependent DNA polymerase and the nCas9 are covalently linked sequentially in a polypeptide comprising from N-terminus to C-terminus the nCas9 and the DNA-dependent DNA polymerase.

Embodiment 285 is the system of Embodiment 284, wherein the at least one chromatin remodeler is positioned N-terminal to the nCas9.

Embodiment 286 is the system of Embodiment 284, wherein the at least one chromatin remodeler is positioned between the nCas9 and the DNA-dependent DNA polymerase.

Embodiment 287 is the system of Embodiment 284, wherein the at least one chromatin remodeler is positioned C-terminal to the DNA-dependent DNA polymerase.

Embodiment 288 is the system of any one of Embodiments 283-287, further comprising a nuclear localization signal (NLS).

Embodiment 289 is the system of any one of Embodiments 283-288, wherein the DNA-dependent DNA polymerase and the nCas9 are covalently linked sequentially in a polypeptide from N-terminus to C-terminus, wherein the order is nuclear localization signal-chromatin remodeler-nCas9-DNA Dependent DNA polymerase-nuclear localization signal.

Embodiment 290 is the system of any one of Embodiments-283-288, wherein the DNA-dependent DNA polymerase and the nCas9 are covalently linked sequentially in a polypeptide from N-terminus to C-terminus, wherein the order is nuclear localization signal-nCas9-chromatin remodeler-DNA Dependent DNA polymerase-nuclear localization signal.

Embodiment 291 is the system of any one of Embodiments 283-288, wherein the DNA-dependent DNA polymerase and the nCas9 are covalently linked sequentially in a polypeptide from N-terminus to C-terminus, wherein the order is nuclear localization signal-nCas9-DNA Dependent DNA polymerase-chromatin remodeler-nuclear localization signal.

Embodiment 292 is the system of any one of Embodiments 289-291, further comprising one or more linkers between the nuclear localization signal(s), nCas9, DNA-dependent DNA polymerase, or the chromatin remodeler.

Embodiment 293 is the system of any one of Embodiments 283-292, wherein at least one chromatin remodeler is selected from a polypeptide comprising HMGB1, HMGN1, HMGN2, HMGN3a, HMGN3b, HMGN4, HMGN5, histone H1.0, histone H1.2, CHD1, ISWI, DNA helicase, TOP1, herpesvirus 8 LANA, CMV IE1 or a domain thereof.

Embodiment 294 is the system of any one of Embodiments 283-293, wherein the at least one chromatin remodeler comprises an HMGB1 polypeptide.

Embodiment 295 is the system of Embodiment 294, wherein the HMGB1 polypeptide comprises an HMGB1 polypeptide, wherein the HMGB1 polypeptide comprises an HMGB1 Box B domain and at least 7 contiguous amino acid residues C-terminal to the HMGB1 Box B domain, and wherein the HMGB1 polypeptide lacks all or part of an acidic tail domain.

Embodiment 296 is the system of Embodiment 294 or 295, wherein the HMGB1 polypeptide comprises a cryptic nuclear localization signal (NLS).

Embodiment 297 is the system of Embodiment 296, wherein the cryptic NLS comprises the sequence of EKSKKKK (SEQ ID NO: 1787).

Embodiment 298 is the system of any one of Embodiments 294-297, wherein the HMGB1 polypeptide comprises a receptor for advanced glycation end-products (RAGE) binding domain.

Embodiment 299 is the system of Embodiment 298, wherein the RAGE binding domain comprises the sequence of SEQ ID NO: 1803.

Embodiment 300 is the system of any one of Embodiments 294-299, wherein the HMGB1 polypeptide comprises 7-20 amino acid residues C-terminal to the HMGB1 Box B domain.

Embodiment 301 is the system of any one of Embodiments 294-300, wherein the polypeptide comprises the sequence of SEQ ID NO: 1804.

Embodiment 302 is the system of any one of Embodiments 294-301, wherein the HMGB1 polypeptide lacks a sequence at least 50%, 60%, 70%, 80%, 90%, 94%, or 97% identical to SEQ ID NO: 1788 or lacks the sequence of SEQ ID NO: 1788.

Embodiment 303 is the system of any one of Embodiments 294-302, wherein the HMGB1 polypeptide lacks transcriptional stimulatory function.

Embodiment 304 is the system of any one of Embodiments 294-303, wherein the HMGB1 polypeptide comprises a cryptic NLS that is located C-terminal to the HMGB1 Box B domain.

Embodiment 305 is the system of any one of Embodiments 294-304, wherein the at least 7 contiguous amino acid residues C-terminal end of the HMGB1 Box B domain comprise the cryptic NLS.

Embodiment 306 is the system of any one of Embodiments 294-305, wherein the cryptic NLS immediately follows the C-terminal end of the HMGB1 Box B domain.

Embodiment 307 is the system of any one of Embodiments 294-306, wherein the HMGB1 polypeptide lacks an HMGB1 Box A domain.

Embodiment 308 is the system of any one of Embodiments 294-307, wherein the HMGB1 polypeptide comprises two HMGB1 Box B domains.

Embodiment 309 is the system of any one of Embodiments 294-308, wherein the cryptic NLS is located C-terminal to the C-terminal end of the two HMGB1 Box B domains.

Embodiment 310 is the system of any one of Embodiments 294-306 and 308-309, wherein the HMGB1 polypeptide comprises an HMGB1 Box A domain.

Embodiment 311 is the system of any one of Embodiments 294-306 and 308-310, wherein the Box B domain is located C-terminal to the HMGB1 Box A domain.

Embodiment 312 is the system of any one of Embodiments 294-306 and 308-311, wherein the HMGB1 polypeptide comprises, from N-terminus to C-terminus:
a) HMGB1 Box A domain-HMGB1 Box B domain-cryptic NLS;
b) HMGB1 Box B domain-HMGB1 Box B domain-cryptic NLS; or
c) HMGB1 Box B domain-cryptic NLS.

Embodiment 313 is the system of any one of Embodiments 294-312, wherein the HMGB1 polypeptide or HMGB1 polypeptide comprises at least 90% sequence identity to SEQ ID NO: 1783-1792 or 1803-1804.

Embodiment 314 is the system of any one of Embodiment 294-313, wherein the HMGB1 polypeptide or HMGB1 polypeptide comprises the amino acid sequence of SEQ ID NO:1783-1792 or 1803-1804.

Embodiment 315 is the system of any one of Embodiments 248-314, further comprising a template guide RNA (tgRNA), wherein the tgRNA comprises a first polynucleotide and a second polynucleotide, wherein:
a) the first polynucleotide of the tgRNA comprises a SpyCas9 guide RNA comprising, from 5′ to 3′:
   1) a spacer that is complementary to a target region of a target strand of a DNA duplex target nucleic acid; and
   2) a scaffold; and
b) the second polynucleotide of the tgRNA is operably linked to the first polynucleotide of the tgRNA and comprises, from 5′ to 3′:
   1) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides;
   2) ii) a DNA-dependent DNA polymerase recruiting sequence (DRS) that is 6-18 nucleotides in length, optionally 8-18 or 10-12 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the nontarget strand of the DNA duplex target nucleic acid.

Embodiment 316 is the system of Embodiment 315, wherein the DRS comprises RNA nucleotides

Embodiment 317 is the system of Embodiment 315 or 316, wherein the 3′ terminal nucleotide in the DRS is an RNA nucleotide.

Embodiment 318 is the system of Embodiment 315, wherein the DRS comprises DNA nucleotides.

Embodiment 319 is the system of Embodiment 315 or 318, wherein the DRS consists of DNA nucleotides.

Embodiment 320 is the system of any one of Embodiments 315-318, wherein the 3′ terminal nucleotide in the DRS is an RNA nucleotide and the remaining nucleotides in the DRS are DNA nucleotides.

Embodiment 321 is the system of any one of Embodiments 316-317 and 320, wherein the RNA nucleotide is a modified RNA nucleotide, optionally wherein the modified nucleotide selected from a 2′-O-methyl (2′-O-Me) modified nucleotide, or a 2′-F modified nucleotide.

Embodiment 322 is the system of any one of Embodiments 315-321, wherein the spacer of the guide RNA comprises a modified nucleotide.

Embodiment 323 is the system of any one of Embodiments 315-322 wherein the spacer of the guide RNA comprises DNA nucleotides.

Embodiment 324 is the system of any one of Embodiments 315-323, wherein the spacer of the guide RNA comprises a DNA nucleotide at least 2, 3, 4, 5, 6, 7, 8, 9, or 10 nucleotides.

Embodiment 325 is the system of any one of Embodiments 315-323, wherein the spacer of the guide RNA comprises a DNA nucleotide at 1, 2, 3, 4, 5, 6, 7, 8, 9, 10, or 11 nucleotides.

Embodiment 326 is the system of any one of Embodiments 315-325, wherein the spacer of the guide RNA comprises a DNA nucleotide at one or more of positions S11, S12, and S18.

Embodiment 327 is the system of any one of Embodiments 315-322, wherein the spacer of the guide RNA does not comprise DNA nucleotides.

Embodiment 328 is the system of any one of Embodiments 315-327, wherein the spacer of the guide RNA comprises a modified nucleotide selected from a 2′-O-methyl (2′-O-Me) modified nucleotide, or a 2′-F modified nucleotide.

Embodiment 329 is the system of any one of Embodiments 315-328, wherein the spacer of the guide RNA comprises a modified nucleotide at one or more of the first five nucleotides at the 5′ end.

Embodiment 330 is the system of any one of Embodiments 315-329, wherein the spacer of the guide RNA comprises a 2′-O-Me modified nucleotide at least 2, 3, 4, 5, 6, 7, 8, 9, or 10 nucleotides.

Embodiment 331 is the system of any one of Embodiments 315-330, wherein the spacer of the guide RNA comprises 2′-O-Me modified nucleotides at one or more of positions S4, S5, S9, S10, S13, S14, S17 and S20.

Embodiment 332 is the system of any one of Embodiments 315-331, wherein the spacer of the guide RNA comprises 2′-O-Me modified nucleotides at each of the first three nucleotides at the 5′ end of the spacer.

Embodiment 333 is the system of any one of Embodiments 319-332, wherein the spacer of the guide RNA comprises 2′-O-Me modified nucleotides at each of S1, S2, S3, S9, S10, S13, S14, S16, and S20, and unmodified nucleotides at each of S6, S7, S8, S15, S16, and S19, optionally unmodified nucleotides at one, two, three, four, or all of S4, S5, S11, S12, and S18.

Embodiment 334 is the system of any one of Embodiments 319-333, wherein the spacer of the guide RNA comprises 2′-O-Me modified nucleotides at each of S1, S2, S3, S9, S10, S13, S14, S17, and S20, DNA nucleotides at each of S11, S12, and S18, and unmodified nucleotides at each of S6, S7, S8, S15, S16, and S19, optionally unmodified nucleotides at one or both of S4 and S5.

Embodiment 335 is A system comprising a DNA polymerase kappa (polK), a SpyCas9 nickase (nCas9), and a template guide RNA (tgRNA), wherein the tgRNA comprises a first polynucleotide and a second polynucleotide, wherein:
a) the first polynucleotide comprises a SpyCas9 guide RNA comprising, from 5′ to 3′:
   1) a spacer that is complementary to a target region of a target strand of a DNA duplex target nucleic acid; and
   2) a scaffold; and
b) the second polynucleotide is operably linked to the first polynucleotide and comprises, from 5′ to 3′:
   1) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides;
   2) a DNA-dependent DNA polymerase recruiting sequence (DRS) that is 6-18 nucleotides in length, optionally 8-18 or 10-12 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid, wherein the DRS comprises RNA nucleotides.

Embodiment 336 is the system of Embodiment 335, wherein the polK comprises an amino acid sequence that has at least 90%, 91%, 92%, 93%, 94%, 95%, 96%, 97%, 98%, 99%, or 100% identity to amino acid residues 19-526 of SEQ ID NO: 1021; or an amino acid sequence that has at least 90%, 91%, 92%, 93%, 94%, 95%, 96%, 97%, 98%, 99%, or 100% identity to SEQ ID NO: 1021, optionally wherein the polk includes or lacks an N-terminal methionine relative to SEQ ID NO: 1021.

Embodiment 337 is the system of Embodiment 335 or 336, wherein the polk comprises amino acids 19-526, optionally amino acids 1-526, of polK relative to SEQ ID NO: 1021.

Embodiment 338 is the system of any one of Embodiments 335-337, wherein the polK comprises a mutation at G411 or E412 relative to SEQ ID NO: 1021, optionally wherein the polK comprises a mutation at G411 and E412.

Embodiment 339 is the system of any one of Embodiments 335-338, wherein the polk comprises GE411-412RV mutations relative to SEQ ID NO: 1021.

Embodiment 340 is the system of any one of Embodiments 335-339, wherein the polypeptide comprises a heterodimerization domain or further comprises an additional heterodimerization domain.

Embodiment 341 is the system of any one of Embodiments 335-340, wherein the polk and nCas9 are covalently linked sequentially in a polypeptide from N-terminus to C-terminus, wherein the order is:
a) NLS-nCas9-linker-NLS-linker-polK; or
b) NLS-nCas9-linker-NLS-linker-polK-linker-NLS-linker-heterodimerization domain.

Embodiment 342 is the system of Embodiment 340 or 341, wherein the heterodimerization domain comprises a coiled coil heterodimerization domain.

Embodiment 343 is the system of any one of Embodiments 340-342, wherein the heterodimerization domain comprises an EV, EI, KV, or KI domain.

Embodiment 344 is the system of any one of Embodiments 340-343, wherein the heterodimerization domain comprises an EI or KI domain.

Embodiment 345 is the system of any one of Embodiments 340-344, wherein the heterodimerization domain comprises SEQ ID NO: 1589, SEQ ID NO: 1593, or SEQ ID NO: 1595.

Embodiment 346 is the system of any one of Embodiments 335-345, wherein polk is encoded by:
a) a nucleotide sequence that has at least 90%, 91%, 92%, 93%, 94%, 95%, 96%, 97%, 98%, 99%, or 100% identity to a coding sequence for amino acid residues 19-526 within SEQ ID NO: 1021;
b) a nucleotide sequence that has at least 90%, 91%, 92%, 93%, 94%, 95%, 96%, 97%, 98%, 99%, or 100% identity to SEQ ID NO: 1020;
c) a nucleotide sequence that has at least 90%, 91%, 92%, 93%, 94%, 95%, 96%, 97%, 98%, 99%, or 100% identity to SEQ ID NO: 1201;
d) a nucleotide sequence that has at least 90%, 91%, 92%, 93%, 94%, 95%, 96%, 97%, 98%, 99%, or 100% identity to SEQ ID NO: 1203;
e) a nucleotide sequence that has at least 90%, 91%, 92%, 93%, 94%, 95%, 96%, 97%, 98%, 99%, or 100% identity to SEQ ID NO: 1205;
f) a nucleotide sequence that has at least 90%, 91%, 92%, 93%, 94%, 95%, 96%, 97%, 98%, 99%, or 100% identity to SEQ ID NO: 1264; or
g) a nucleotide sequence that has at least 90%, 91%, 92%, 93%, 94%, 95%, 96%, 97%, 98%, 99%, or 100% identity to SEQ ID NO: 1018.

Embodiment 347 is the system of any one of Embodiments 335-346, wherein the polK is encoded by a nucleotide sequence that has at least 90%, 91%, 92%, 93%, 94%, 95%, 96%, 97%, 98%, 99%, or 100% identity to SEQ ID NO: 1020, optionally wherein the nucleotide sequence includes or lacks a start codon or a stop codon relative to SEQ ID NO: 1020.

Embodiment 348 is the system of any one of Embodiments 335-347, wherein the polK is encoded by a nucleotide sequence that has at least 90%, 91%, 92%, 93%, 94%, 95%, 96%, 97%, 98%, 99%, or 100% identity to SEQ ID NO: 1201.

Embodiment 349 is the system of any one of Embodiments 335-348, wherein the template comprises 1-3 RNA nucleotides, optionally only 1 RNA nucleotide, at the 3′ end of the template.

Embodiment 350 is the system of any one of Embodiments 335-349, wherein the template consists of DNA nucleotides from nucleotide 4 (T4) from the 3′ end of the template to the 5′ end of the template.

Embodiment 351 is the system of any one of Embodiments 335-348, wherein the template consists of DNA nucleotides.

Embodiment 352 is the system of any one of Embodiments 335-351, wherein the DRS comprises a 3′ end and a 5′ end, wherein one or more of nucleotides 1-7 nucleotides at the 5′ end of the DRS (nucleotides D1-D7) are DNA nucleotides.

Embodiment 353 is the system of any one of Embodiments 335-352, wherein one or more of nucleotides 1-5 at the 5′ end of the DRS (nucleotides D1-D5) are DNA nucleotides, optionally wherein the remaining nucleotides in the DRS are RNA nucleotides.

Embodiment 354 is the system of any one of Embodiments 335-353, wherein one or more of nucleotides 1-3 at the 5′ end of the DRS (nucleotides D1-D3), optionally the 5′ terminal nucleotide in the DRS (nucleotide D1), is a DNA nucleotide.

Embodiment 355 is the system of any one of Embodiments 335-354, wherein nucleotides 1 and 3 at the 5′ end of the DRS (nucleotides D1 and D3) are DNA nucleotides, or wherein nucleotides 1, 3, and 5 at the 5′ end of the DRS (nucleotides D1, D3, and D5) are DNA nucleotides, optionally wherein the remaining nucleotides in the DRS are RNA nucleotides.

Embodiment 356 is the system of any one of Embodiments 335-355, wherein the DRS comprises a sequence that is complementary to a DRS-complementary region of a non-target strand of the DNA duplex target nucleic acid comprising nucleotides D2-D9 of the DRS, optionally D2-D8 or D3-D9 of the DRS.

Embodiment 357 is the system of any one of Embodiments 335-356, wherein the DRS comprises a sequence that is complementary to a DRS-complementary region of a non-target strand of the DNA duplex target nucleic acid comprising nucleotides D6-D13 of the DRS, optionally wherein 1, 2, 3, 4, 5, 6, 7, or 8 of nucleotides D6-D13 are DNA nucleotides.

Embodiment 358 is the system of any one of Embodiments 335-357, wherein the 3′ end of the first polynucleotide is covalently linked to the 5′ end of the second polynucleotide by a phosphorothioate or phosphodiester bond.

Embodiment 359 is the system of Embodiment 358, wherein the 5′ terminal nucleotide in the DRS (D1) is a DNA nucleotide.

Embodiment 360 is the system of Embodiment 359, wherein the penultimate 5′ nucleotide (D2) in the DRS is an RNA nucleotide.

Embodiment 361 is the system of Embodiment 359, wherein each nucleotide from the penultimate 5′ nucleotide (D2) to the 3′ end nucleotide in the DRS is an RNA nucleotide.

Embodiment 362 is the system of any one of Embodiments 335-361, wherein the 3′ terminal nucleotide in the DRS is an RNA nucleotide and the remaining nucleotides in the DRS are DNA nucleotides.

Embodiment 363 is the system of any one of Embodiments 335-357, wherein the first polynucleotide is not covalently linked to the second polynucleotide.

Embodiment 364 is the system of any one of Embodiments 335-357 or 363, wherein 1-7 consecutive 5′ terminal nucleotides of the DRS are DNA nucleotides.

Embodiment 365 is the system of any one of Embodiments 335-357 or 363-364, wherein the 5′ terminal and penultimate 5′ terminal nucleotides of the DRS are DNA nucleotides.

Embodiment 366 is the system of any one of Embodiments 335-357 or 363-365, wherein at least 3 consecutive 5′ terminal nucleotides of the DRS are DNA nucleotides.

Embodiment 367 is the system of any one of Embodiments 335-357 or 363-366, wherein 3 consecutive 5′ terminal nucleotides of the DRS are DNA nucleotides.

Embodiment 368 is the system of any one of Embodiments 335-367, wherein the DRS comprises a 3′ tail at the 3′ end of the DRS, wherein the tail is optionally 1-3 nucleotides in length.

Embodiment 369 is the system of Embodiment 368, wherein the 3′ tail of the DRS comprises an RNA nucleotide.

Embodiment 370 is the system of Embodiment 368 or 369, wherein the 3′ tail comprises a uridine nucleotide, optionally wherein the 3′ terminal nucleotide is a modified uridine nucleotide.

Embodiment 371 is the system of any one of Embodiments 335-370, wherein the RNA nucleotide comprises a modified RNA nucleotide.

Embodiment 372 is the system of any one of Embodiments 335-371, wherein each modified RNA nucleotide is independently selected from:
1) 2′-O-methyl (2′-O-Me) modified nucleotide;
2) 2′-fluoro (2′-F) modified nucleotide;
3) an amine modified nucleotide;
4) an alkoxy modified nucleotide;
5) an LNA modified nucleotide;
6) methoxyethyl modified nucleotide;
7) a halo modified nucleotide; or 8 phosphorothioate (PS) linkage modified nucleotide;
   optionally, wherein the modified nucleotide is a 2′-O-methyl (2′-O-Me) modified nucleotide or a phosphorothioate (PS) linkage modified nucleotide.

Embodiment 373 is the system of any one of Embodiments 335-372, wherein the 3′ terminal nucleotide of the DRS comprises a 2′-O-Me modification.

Embodiment 374 is the system of any one of Embodiments 335-373, wherein the DRS comprises PS linkages between 2, 3, 4, 5, or 6 consecutive 3′ terminal nucleotides of the DRS

Embodiment 375 is the system of any one of Embodiments 335-374, wherein the DRS comprises a PS linkage between the 3′ terminal and penultimate 3′ terminal nucleotides of the DRS.

Embodiment 376 is the system of any one of Embodiments 335-375, wherein the DRS comprises 3 PS linkages between the 4 consecutive 3′ terminal nucleotides of the DRS.

Embodiment 377 is the system of any one of Embodiments 335-376, wherein the DRS comprises a PS linkage between 2 nucleotides other than the 3′ terminal and penultimate 3′ terminal nucleotides of the DRS.

Embodiment 378 is the system of any one of Embodiments 335-377, wherein the DRS comprises a 2′-O-Me modification at a nucleotide other than the 5′ terminal and 3′ terminal nucleotides of the DRS.

Embodiment 379 is the system of any one of Embodiments 335-378, wherein the DRS comprises a phosphorothioate modification, and wherein the DRS does not comprise a phosphorothioate modification at position D1-D9 in the DRS sequence.

Embodiment 380 is the system of any one of Embodiments 335-379, wherein the DRS comprises a phosphorothioate modification at or 3′ of position D10 in the DRS sequence.

Embodiment 381 is the system of any one of Embodiments 335-380, wherein the DRS comprises a 2′-O-Me modification, and wherein the DRS does not comprise a 2′-O-Me modification at position D1-D3 in the DRS sequence.

Embodiment 382 is the system of any one of Embodiments 335-381, wherein the DRS comprises a 2′-O-Me modification at or 3′ of position D4 in the DRS sequence.

Embodiment 383 is the system of any one of Embodiments 335-382, wherein the DRS comprises a sequence that is not fully complementary to the DRS-complementary region of the non-target strand of the DNA duplex target nuclei acid.

Embodiment 384 is the system of any one of Embodiments 335-383, wherein the template comprises a mismatch to the genomic sequence, optionally 1-4 mismatches to the genomic sequence.

Embodiment 385 is the system of Embodiment 384, wherein the mismatch that directs a template-based edit in the genomic DNA is within nucleotides T1-T25, optionally within nucleotides T1-T15 of the template.

Embodiment 386 is the system of Embodiment 385, wherein the template-based edit in the genomic DNA comprises a substitution.

Embodiment 387 is the system of Embodiment 385 or 386, wherein the template-based edit in the genomic DNA comprises an insertion of a nucleotide in the genomic sequence.

Embodiment 388 is the system of Embodiment 385 or 386, wherein the template-based edit in the genomic DNA comprises a deletion of a nucleotide in the genomic sequence.

Embodiment 389 is the system of any one of Embodiments 385-387, wherein the template-based edit in the genomic sequence results in a change in an amino acid sequence encoded by a locus in which the genomic sequence is present.

Embodiment 390 is the system of any one of Embodiments 385-387, wherein the template-based edit in the genomic sequence does not result in a change in an amino acid sequence encoded by a locus in which the genomic sequence is present.

Embodiment 391 is the system of any one of Embodiments 335-390, wherein the polK or the nCas9 is linked to a heterologous nuclear localization signal (NLS).

Embodiment 392 is the system of any one of Embodiments 335-391, wherein the polk is linked to an MS2 coat protein (MCP) domain.

Embodiment 393 is the system of any one of Embodiments 335-392, wherein the polk is covalently linked to the nCas9.

Embodiment 394 is the system of Embodiment 393, wherein the polK is covalently linked to the nCas9 in sequence from N-terminus to C-terminus, polk-nCas9.

Embodiment 395 is the system of Embodiment 393, wherein the polK is covalently linked to the nCas9 in sequence from N-terminus to C-terminus, nCas9-polK.

Embodiment 396 is the system of any one of Embodiments 393-395, wherein the polk and nCas9 are covalently linked with a peptide linker.

Embodiment 397 is the system of any one of Embodiments 335-396 wherein the polk and nCas9 are covalently linked sequentially in a polypeptide from N-terminus to C-terminus in an order selected from:
a) NLS-nCas9-NLS-polK;
b) NLS-nCas9-linker-NLS-linker-polK;
c) polK-NLS-nCas9-NLS;
d) polK-linker-NLS-linker-nCas9-linker-NLS;
e) NLS-nCas9-NLS-polK(GE411-412RV);
f) NLS-nCas9-linker-NLS-polK(GE411-412RV);
g) NLS-nCas9-NLS-polK(19-526);
h) NLS-nCas9-linker-NLS-linker-polK(19-526);
i) polK(19-526)-NLS-nCas9-NLS;
j) polK(19-526)-Linker-NLS-linker-nCas9-linker-NLS;
k) polK(GE411-412RV)-NLS-NLS-nCas9;
l) polK(GE411-412RV)-linker-NLS-linker-NLS-nCas9;
m) NLS-nCas9-NLS-polK(19-526, GE411-412RV);
n) NLS-nCas9-linker-NLS-linker-polK(19-526, GE411-412RV);
o) NLS-nCas9-NLS-polK-NLS;
p) NLS-nCas9-linker-NLS-linker-polK-linker-NLS;
q) NLS-nCas9-NLS-polK(1-526, GE411-412RV)-NLS; or
r) NLS-nCas9-linker-NLS-linker-polK(1-526, GE411-412RV)-linker-NLS;
s) nCas9-polK(1-526);
t) polK(1-526, GE411-412RV)-nCas9-NLS-heterodimerization domain, optionally wherein the heterodimerization domain comprises a coiled coil heterodimerization domain, optionally an EI domain or a KI domain;
u) polK(1-526, GE411-412RV)-linker-NLS-nCas9-linker-NLS-heterodimerization domain,
optionally wherein the heterodimerization domain comprises a coiled coil heterodimerization domain, optionally an EI domain or a KI domain;
optionally wherein the order is NLS-nCas9-linker-NLS-linker-polK(1-526, GE411-412RV)-linker-NLS, optionally further comprising a heterodimerization domain comprising a coiled coil heterodimerization domain, optionally an EI domain or a KI domain.

Embodiment 398 is the system of any one of Embodiments 335-397, wherein the DNA-dependent DNA polymerase and nCas9 are provided as one or more polypeptides.

Embodiment 399 is the system of any one of Embodiments 335-397, wherein the DNA-dependent DNA polymerase and nCas9 are provided as one or more nucleic acids.

Embodiment 400 is the system of any one of Embodiments 335-399, wherein the polK, or portion thereof, is operably linked to a first split-intein and the nCas9, or portion thereof, is operably linked to a second split-intein that is complementary to the first split intein, wherein the first split-intein and second split-intein undergo trans-splicing reaction, resulting in the formation of a single fusion polypeptide comprising, from N-terminus to C-terminus, (i) the polk and the nCas9 or (2) the polk and the nCas9.

Embodiment 401 is the system of any one of Embodiments 400, wherein the first split-intein and the second split-intein are capable of binding to each other to undergo intein-mediated protein splicing.

Embodiment 402 is the system of any one of Embodiments 400 or 401, wherein the first split-intein is an N-split-intein and the second split-intein is a C-split intein.

Embodiment 403 is the system of any one of Embodiments 400 or 401, wherein the first split-intein is a C-split-intein and the second split intein is an N-split-intein.

Embodiment 404 is the system of any one of Embodiments 400-403, wherein the first split-intein and second split-intein are complementary inteins selected from Cfa, M86, Npu, NpuSsp, gp41-1, gp41-8, NrdJ-1, IMPDH-1, SspDnaX, SspGyrB, Cth-Ter, NrdA-2, Mja-KlbA, RBS1, TE3S11, and Rma.

Embodiment 405 is the system of any one of Embodiments 400-404, wherein the first split-intein and second split-intein are Cfa-inteins.

Embodiment 406 is the system of any one of Embodiments 405, wherein the first split-intein is a Cfa C-split-intein and the second split-intein is a Cfa N-split-intein.

Embodiment 407 is the system of any one of Embodiments 405, wherein the Cfa C-split-intein comprises an amino acid sequence having at least 90% identity to SEQ ID NO: 1746 and the Cfa N-split-intein comprises an amino acid sequence having at least 90% identity to SEQ ID NO: 1744.

Embodiment 408 is the system of any one of Embodiments 407, wherein the Cfa C-split-intein comprises the amino acid sequence of SEQ ID NO: 1746 and the Cfa N-split-intein comprises the amino acid sequence of SEQ ID NO: 1744.

Embodiment 409 is the system of any one of Embodiments 400-408, wherein the polk is operably linked to the first split-intein by a first peptide linker.

Embodiment 410 is the system of Embodiment 409, wherein the first peptide linker comprises 30 amino acids, optionally wherein the first peptide linker comprises an amino acids sequence at least 90%, 95%, 98%, or 100% identical to SEQ ID NO: 1829.

Embodiment 411 is the system of any one of Embodiments 400-410, wherein the nCas9 is operably linked to the second split-intein by a second peptide linker, optionally, wherein the second peptide linker is SGGS (SEQ ID NO: 1738).

Embodiment 412 is the system of any one of Embodiments 400-411, wherein the polK is operably linked to a heterologous nuclear localization signal (NLS).

Embodiment 413 is the system of any one of Embodiments 400-412, wherein the nCas9 is operably linked to a heterologous nuclear localization signal (NLS).

Embodiment 414 is the system of Embodiment 413, wherein the heterologous nuclear localization signal is selected from an SV40 NLS, c-Myc NLS, and nucleoplasmin NLS.

Embodiment 415 is the system of any one of Embodiments 335-414, wherein the polK and nCas9 are operably linked to at least one chromatin remodeler.

Embodiment 416 is the system of Embodiment 415, wherein the polk, nCas9, and chromatin remodeler are covalently linked sequentially in a polypeptide from N-terminus to C-terminus in an order selected from:
a) chromatin remodeler-nCas9-polK;
b) nCas9-chromatin remodeler-polK; or
c) nCas9-polK-chromatin remodeler.
optionally further comprising a nuclear localization signal (NLS)

Embodiment 417 is the system of Embodiment 416, wherein the polk, nCas9, and chromatin remodeler are covalently linked sequentially in a polypeptide from N-terminus to C-terminus in an order selected from:
a) NLS-chromatin remodeler-nCas9-polK-NLS;
b) NLS-chromatin remodeler-nCas9-NLS-polK-NLS;
c) NLS-nCas9-chromatin remodeler-polK-NLS; or
d) nCas9-polK-chromatin remodeler-NLS.

Embodiment 418 is the system of Embodiment 416 or 417, further comprising one or more linkers between the NLS, nCas9, polK, and the chromatin remodeler.

Embodiment 419 is the system of any one of Embodiments 414-418, wherein the at least one chromatin remodeler is selected from a polypeptide comprising HMGB1, HMGN1, HMGN2, HMGN3a, HMGN3b, HMGN4, HMGN5, histone H1.0, histone H1.2, CHD1, ISWI, DNA helicase, TOP1, herpesvirus 8 LANA, CMV IE1 or a domain thereof.

Embodiment 420 is the system of any one of Embodiments 414-419, wherein the at least one chromatin remodeler comprises an HMGB1 polypeptide.

Embodiment 421 is the system of Embodiment 420, wherein the HMGB1 polypeptide comprises a sequence at least 90%, 91%, 92%, 93%, 94%, 95%, 96%, 97%, 98%, 99%, or 100% identical to SEQ ID NO: 1783.

Embodiment 422 is the system of Embodiment 420 or 421, wherein the HMGB1 polypeptide comprises an HMGB1 polypeptide, wherein the HMGB1 polypeptide comprises an HMGB1 Box B domain and at least 7 contiguous amino acid residues C-terminal to the HMGB1 Box B domain, and wherein the HMGB1 polypeptide lacks all or part of an acidic tail domain.

Embodiment 423 is the system of any one of Embodiments 420-422, wherein the HMGB1 polypeptide comprises a cryptic nuclear localization signal (NLS).

Embodiment 424 is the system of Embodiment 423, wherein the cryptic NLS comprises the sequence of EKSKKKK (SEQ ID NO: 1787).

Embodiment 425 is the system of any one of Embodiments 420-424, wherein the HMGB1 polypeptide comprises a receptor for advanced glycation end-products (RAGE) binding domain.

Embodiment 426 is the system of Embodiment 425, wherein the RAGE binding domain comprises the sequence of SEQ ID NO: 1803.

Embodiment 427 is the system of any one of Embodiments 420-426, wherein the HMGB1 polypeptide comprises 7-20 amino acid residues C-terminal to the HMGB1 Box B domain.

Embodiment 428 is the system of any one of Embodiments 420-427, wherein the HMGB1 polypeptide comprises the sequence of SEQ ID NO: 1804.

Embodiment 429 is the system of any one of Embodiments 420-428, wherein the HMGB1 polypeptide lacks a sequence at least 50%, 60%, 70%, 80%, 90%, 94%, or 97% identical to SEQ ID NO: 1788 or lacks the sequence of SEQ ID NO: 1788.

Embodiment 430 is the system of any one of Embodiments 420-429, wherein the HMGB1 polypeptide lacks transcriptional stimulatory function.

Embodiment 431 is the system of any one of Embodiments 420-430, wherein the HMGB1 polypeptide comprises a cryptic NLS that is located C-terminal to the HMGB1 Box B domain.

Embodiment 432 is the system of any one of Embodiments 420-431, wherein the at least 7 contiguous amino acid residues C-terminal end of the HMGB1 Box B domain comprise the cryptic NLS.

Embodiment 433 is the system of any one of Embodiments 420-432, wherein the cryptic NLS immediately follows the C-terminal end of the HMGB1 Box B domain.

Embodiment 434 is the system of any one of Embodiments 420-433, wherein the HMGB1 polypeptide lacks an HMGB1 Box A domain.

Embodiment 435 is the system of any one of Embodiments 420-434, wherein the HMGB1 polypeptide comprises two HMGB1 Box B domains.

Embodiment 436 is the system of any one of Embodiments 420-435, wherein the cryptic

NLS is located C-terminal to the C-terminal end of the two HMGB1 Box B domains.

Embodiment 437 is the system of any one of Embodiments 420-433 or 435-436, wherein the HMGB1 polypeptide comprises an HMGB1 Box A domain.

Embodiment 438 is the system of any one of Embodiments 420-433 or 435-437, wherein the Box B domain is located C-terminal to the HMGB1 Box A domain.

Embodiment 439 is the system of any one of Embodiments 420-433 or 435-438, wherein the HMGB1 polypeptide comprises, from N-terminus to C-terminus:
a) HMGB1 Box A domain-HMGB1 Box B domain-cryptic NLS;
b) HMGB1 Box B domain-HMGB1 Box B domain-cryptic NLS; or
c) HMGB1 Box B domain-cryptic NLS.

Embodiment 440 is the system of any one of Embodiments 420-439, wherein the HMGB1 polypeptide comprises at least 90% sequence identity to SEQ ID NO: 1783-1792 and 1803-1804 or the amino acid sequence of SEQ ID NO: 1783-1792 and 1803-1804.

Embodiment 441 is the system of any one of Embodiments 335-397, 398, 399, or 415-440, wherein the polk and nCas9 are not covalently linked, and the polK is provided with a 3′ extension recruiting domain, wherein, the polK and the 3′ extension recruiting domain are covalently linked sequentially in a polypeptide from N-terminus to C-terminus in an order selected from:
a) 3′ extension recruiting domain-polK-NLS;
b) 3′ extension recruiting domain-linker-polK-linker-NLS;
c) 3′ extension recruiting domain-polK(GE411-412RV)-NLS; and
d) 3′ extension recruiting domain-linker-polK(GE411-412RV)-linker-NLS.

Embodiment 442 is the system of Embodiment 397-441, wherein the polypeptide comprises a peptide linker or further comprises an additional peptide linker.

Embodiment 443 is the system of any one of Embodiments Embodiment 441 or 442, wherein the polypeptide comprises a heterodimerization domain or further comprises an additional heterodimerization domain.

Embodiment 444 is the system of Embodiment 443, wherein polK and nCas9 are covalently linked sequentially in a polypeptide from N-terminus to C-terminus, wherein the order is NLS-nCas9-linker-NLS-linker-polK (1-526, GE411-412RV)-linker-NLS-linker-heterodimerization domain.

Embodiment 445 is the system of any one of Embodiments 443 or 444, wherein polK and nCas9 are covalently linked sequentially in a polypeptide from N-terminus to C-terminus, wherein the order is:
a) NLS-nCas9-linker-NLS-linker-polK(1-526, GE411-412RV)-linker-NLS-linker-EI domain; or
b) NLS-nCas9-linker-NLS-linker-polK(1-526, GE411-412RV)-linker-NLS-linker-KI domain.

Embodiment 446 is the system of any one of Embodiments 443-445, wherein the heterodimerization domain comprises a coiled coil heterodimerization domain.

Embodiment 447 is the system of any one of Embodiments 443-446, wherein the heterodimerization domain comprises an EV, EI, KV, or KI domain.

Embodiment 448 is the system of any one of Embodiments 443-447, wherein the heterodimerization domain comprises an EI or KI domain.

Embodiment 449 is the system of any one of Embodiments 443-448, wherein the heterodimerization domain comprises SEQ ID NO: 1589, SEQ ID NO: 1593, or SEQ ID NO: 1595.

Embodiment 450 is the system of any one of Embodiments 443-449, wherein the NLS is selected from c-Myc, SV40, nucleoplasmin, or snurportin-1 importin-β NLS, optionally wherein the NLS is an SV40 NLS.

Embodiment 451 is the system of Embodiment 450, wherein polk and nCas9 are covalently linked sequentially in a polypeptide from N-terminus to C-terminus, wherein the order is:
a) SV40 NLS-nCas9-linker-SV40 NLS-linker-polK (1-526, GE411-412RV)-linker-SV40 NLS;
b) SV40 NLS-nCas9-linker-SV40 NLS-linker-polK (1-526, GE411-412RV)-linker-SV40 NLS-linker-EI domain; or
c) SV40 NLS-nCas9-linker-SV40 NLS-linker-polK (1-526, GE411-412RV)-linker-SV40 NLS-linker-KI domain.

Embodiment 452 is the system of any one of Embodiments 397-451, wherein the polypeptide comprises a sequence selected from any one of SEQ ID NOs: 1024, 1027, 1030, 1036, 1039, 1042, 1045, 1048, 1053, 1056, 1059, 1074, 1200, 1202, 1229, 1234, or 1268, or 1300, 1373, 1388, 1447, 1453, 1923, 1926, or 1929.

Embodiment 453 is the system of any one of Embodiments 397-451, wherein the polypeptide is encoded by a nucleotide sequence that has at least 90%, 91%, 92%, 93%, 94%, 95%, 96%, 97%, 98%, 99%, or 100% identity to any one of SEQ ID NOs: 1023, 1026, 1029, 1035, 1038, 1041, 1044, 1047, 1052, 1055, 1058, 1073, 1199, 1201, 1228, 1233, or 1267, and 1299, 1372, 1387, 1446, 1452, 1922, 1925, or 1928.

Embodiment 454 is the system of any one of Embodiments 397-453, wherein the polypeptide comprises a sequence selected from any one of SEQ ID NOs: 1062 and 1067.

Embodiment 455 is the system of any one of Embodiments 397-454, wherein the polypeptide is encoded by a nucleotide sequence that has at least 90%, 91%, 92%, 93%, 94%, 95%, 96%, 97%, 98%, 99%, or 100% identity to any one of SEQ ID NOs: 1061 and 1066.

Embodiment 456 is a system comprising a T5 DNA polymerase (T5 pol), a SpyCas9 nickase (nCas9), and a template guide RNA (tgRNA), wherein the tgRNA comprises a first polynucleotide and a second polynucleotide, wherein:
a) the first polynucleotide comprises a SpyCas9 guide RNA comprising, from 5′ to 3′:
   1) a spacer that is complementary to a target region of a target strand of a DNA duplex target nucleic acid; and
   2) a scaffold; and
b) the second polynucleotide is operably linked to the first polynucleotide and comprises, from 5′ to 3′:
   1) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; and
   2) a DNA-dependent DNA polymerase recruiting sequence (DRS) that is 6-20 nucleotides in length, optionally 10-16 or 12-14 nucleotides in length, and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid comprising nucleotides 5-10 5′ of the PAM in the non-target strand of the DNA duplex target nucleic acid, wherein the DRS comprises RNA nucleotides.

Embodiment 457 is the system of Embodiment 456, wherein the T5 pol comprises an amino acid sequence that has at least 90%, 91%, 92%, 93%, 94%, 95%, 96%, 97%, 98%, 99%, or 100% identity to any one of SEQ ID NO: 1116 or SEQ ID NO: 1121, optionally wherein the T5 pol includes or lacks an N-terminal methionine relative to SEQ ID NO: 1116 or 1121.

Embodiment 458 is the system of Embodiment 456 or 457, wherein the T5 pol comprises a mutation at D164 or E166 relative to SEQ ID NO: 1116.

Embodiment 459 is the system of any one of Embodiments 456-458, wherein the T5 pol comprises D164A and E166A mutations relative to SEQ ID NO: 1116.

Embodiment 460 is the system of any one of claims 456-459, wherein the T5 pol comprises an amino acid sequence comprising an N-terminal deletion of 30 amino acids relative to SEQ ID NO: 1116.

Embodiment 461 is the system of any one of Embodiments 456-460, wherein the T5 pol is encoded by a nucleotide sequence that has at least 90%, 91%, 92%, 93%, 94%, 95%, 96%, 97%, 98%, 99%, or 100% identity to any one of SEQ ID NOs: 1115 or SEQ ID NO: 1120, optionally wherein the T5 pol includes or lacks a start or stop codon relative to SEQ ID NO: 1115 or 1120.

Embodiment 462 is the system of Embodiment 456-461, wherein the template comprises 0-1 RNA nucleotides, optionally 1 RNA nucleotide, at the 3′ end of the template.

Embodiment 463 is the system of any one of Embodiments 456-462, wherein the 3′ terminal nucleotide in the DRS is an RNA nucleotide and the remaining nucleotides in the DRS are DNA nucleotides.

Embodiment 464 is the system of Embodiment 462 or 463, wherein the template consists of DNA nucleotides.

Embodiment 465 is the system of any one of Embodiments 456-464, wherein the DRS comprises a 3′ end and a 5′ end, wherein 0-7 nucleotides at the 5′ end of the DRS (nucleotides D1-D7) are DNA nucleotides.

Embodiment 466 is the system of any one of Embodiments 456-465, wherein one or more of nucleotides 1-5 at the 5′ end of the DRS (nucleotides D1-D5) are DNA nucleotides, optionally wherein the remaining nucleotides in the DRS are RNA nucleotides.

Embodiment 467 is the system of any one of Embodiments 456-466, wherein 1-3 nucleotides at the 5′ end of the DRS (nucleotides D1-D3), optionally the most 5′ nucleotide in the DRS (nucleotide D1), is a DNA nucleotide.

Embodiment 468 is the system of any one of Embodiments 456-467, wherein nucleotides 1 and 3 at the 5′ end of the DRS (nucleotides D1 and D3) are DNA nucleotides, optionally wherein the remaining nucleotides in the DRS are RNA nucleotides.

Embodiment 469 is the system of any one of Embodiments 456-468, wherein nucleotides 1, 3, and 5 at the 5′ end of the DRS (nucleotides D1, D3, and D5) are DNA nucleotides, optionally wherein the remaining nucleotides in the DRS are RNA nucleotides.

Embodiment 470 is the system of any one of Embodiments 456-469, wherein the sequence of the DRS that is complementary to the DRS-complementary region comprises nucleotides D2-D9 of the DRS, optionally D2-D8 or D3-D9 of the DRS.

Embodiment 471 is the system of any one of Embodiments 456-469, wherein the DRS comprises a sequence that is complementary to a DRS-complementary region of a non-target strand of the DNA duplex target nucleic acid comprising nucleotides D6-D13 of the DRS, optionally wherein 1, 2, 3, 4, 5, 6, 7, or 8 of nucleotides D6-D13 are DNA nucleotides.

Embodiment 472 is the system of any one of Embodiments 456-471, wherein the 3′ end of the first polynucleotide is covalently linked to the 5′ end of the second polynucleotide by a phosphorothioate or phosphodiester bond.

Embodiment 473 is the system of any one of Embodiments 456-472, wherein the 5′ terminal nucleotide in the DRS (D1) is a DNA nucleotide.

Embodiment 474 is the system of any one of Embodiments 456-473, wherein the penultimate 5′ nucleotide (D2) in the DRS is an RNA nucleotide.

Embodiment 475 is the system of Embodiment 474, wherein each nucleotide from the penultimate 5′ terminal nucleotide (D2) to the 3′ end nucleotide in the DRS is an RNA nucleotide.

Embodiment 476 is the system of any one of Embodiments 456-471, wherein the first polynucleotide is not covalently linked to the second polynucleotide.

Embodiment 477 is the system of any one of Embodiments 456-471 or 476, wherein 1-7 consecutive 5′ terminal nucleotides of the DRS are DNA nucleotides.

Embodiment 478 is the system of any one of Embodiments 456-471 or 476-477, wherein the 5′ terminal and penultimate 5′ terminal nucleotides of the DRS are DNA nucleotides.

Embodiment 479 is the system of any one of Embodiments 456-471 or 476-478, wherein at least 3 consecutive 5′ terminal nucleotides of the DRS are DNA nucleotides.

Embodiment 480 is the system of any one of Embodiments 456-471 or 476-479, wherein 3 consecutive 5′ terminal nucleotides of the DRS are DNA nucleotides.

Embodiment 481 is the system of any one of Embodiments 456-480, wherein the DRS comprises a 3′ tail at the 3′ end of the DRS, wherein the tail is optionally 1-3 nucleotides in length.

Embodiment 482 is the system of Embodiment 481, wherein the 3′ tail of the DRS comprises an RNA nucleotide.

Embodiment 483 is the system of Embodiment 481 or 482, wherein the 3′ tail comprises a uridine nucleotide, optionally wherein the 3′ terminal nucleotide is a modified uridine nucleotide.

Embodiment 484 is the system of any one of Embodiments 456-483, wherein the RNA nucleotide comprises a modified RNA nucleotide.

Embodiment 485 is the system of any one of Embodiments 456-484, wherein each modified RNA nucleotide is independently selected from:
a) 2′-O-methyl (2′-O-Me) modified nucleotide;
b) 2′-fluoro (2′-F) modified nucleotide;
c) an amine modified nucleotide;
d) an alkoxy modified nucleotide;
e) an LNA modified nucleotide;
f) methoxyethyl modified nucleotide;
g) a halo modified nucleotide; or
h) phosphorothioate (PS) linkage modified nucleotide;
optionally, wherein the modified nucleotide is a 2′-O-methyl (2′-O-Me) modified nucleotide or a phosphorothioate (PS) linkage modified nucleotide.

Embodiment 486 is the system of any one of Embodiments 456-485, wherein the 3′ terminal nucleotide of the DRS comprises a 2′-O-Me modification.

Embodiment 487 is the system of any one of Embodiments 456-486, wherein the DRS comprises PS linkages between 2, 3, 4, 5, or 6 consecutive 3′ terminal nucleotides of the DRS.

Embodiment 488 is the system of any one of Embodiments 456-487, wherein the DRS comprises a PS linkage between the 3′ terminal and penultimate 3′ terminal nucleotides of the DRS.

Embodiment 489 is the system of any one of Embodiments 456-488, wherein the DRS comprises three PS linkages between the 4 consecutive 3′ terminal nucleotides of the DRS.

Embodiment 490 is the system of any one of Embodiments 456-489, wherein the DRS comprises a PS linkage between two nucleotides other than the 3′ terminal and penultimate 3′ terminal nucleotides of the DRS.

Embodiment 491 is the system of any one of Embodiments 456-490, wherein the DRS comprises a 2′-O-Me modification at a nucleotide other than the 5′ terminal and 3′ terminal nucleotides of the DRS.

Embodiment 492 is the system of any one of Embodiments 456-491, wherein the DRS comprises a phosphorothioate modification, and wherein the DRS does not comprise a phosphorothioate modification at position D1-D9 in the DRS sequence.

Embodiment 493 is the system of any one of Embodiments 456-492, wherein the DRS comprises a phosphorothioate modification at or 3′ of position D10 in the DRS sequence.

Embodiment 494 is the system of any one of Embodiments 456-493, wherein the DRS comprises a 2′-O-Me modification, and wherein the DRS does not comprise a 2′-O-Me modification at position D1-D3 in the DRS sequence.

Embodiment 495 is the system of any one of Embodiments 456-494, wherein the DRS comprises a 2′-O-Me modification at or 3′ of position D4 in the DRS sequence.

Embodiment 496 is the system of any one of Embodiments 456-495, wherein the DRS comprises a sequence that is not fully complementary to the DRS-complementary region of the non-target strand of the DNA duplex target nucleic acid.

Embodiment 497 is the system of any one of Embodiments 456-496, wherein the template comprises a mismatch to the genomic sequence, optionally 1-4 mismatches to the genomic sequence.

Embodiment 498 is the system of Embodiment 497, wherein the mismatch that directs a template-based edit in the genomic DNA is within nucleotides T1-T25, optionally within nucleotides T1-T15 of the template.

Embodiment 499 is the system of Embodiment 498, wherein the template-based edit in the genomic DNA comprises a substitution.

Embodiment 500 is the system of Embodiment 498 or 499, wherein the template-based edit in the genomic DNA comprises an insertion of a nucleotide in the genomic sequence.

Embodiment 501 is the system of Embodiment 498 or 499, wherein the template-based edit in the genomic DNA comprises a deletion of a nucleotide in the genomic sequence.

Embodiment 502 is the system of any one of Embodiments 498-501, wherein the template-based edit in the genomic sequence results in a change in an amino acid sequence encoded by a locus in which the genomic sequence is present.

Embodiment 503 is the system of any one of Embodiments 498-501, wherein the template-based edit in the genomic sequence does not result in a change in an amino acid sequence encoded by a locus in which the genomic sequence is present.

Embodiment 504 is the system of any one of Embodiments 456-503, wherein the T5 pol or the nCas9 is linked to a heterologous nuclear localization signal (NLS).

Embodiment 505 is the system of any one of Embodiments 456-504, wherein the T5 pol is linked to an MS2 coat protein (MCP domain).

Embodiment 506 is the system of any one of Embodiments 456-505, wherein the T5 pol is covalently linked to the nCas9.

Embodiment 507 is the system of Embodiment 506, wherein the T5 pol is covalently linked to the nCas9 in sequence from N-terminus to C-terminus, nCas9-T5 pol.

Embodiment 508 is the system of Embodiment 506 or 507, wherein the T5 pol and nCas9 are covalently linked with a peptide linker.

Embodiment 509 is the system of any one of Embodiments 456-508, wherein the T5 pol and nCas9 are covalently linked sequentially in a polypeptide from N-terminus to C-terminus in an order selected from:
a) nCas9-T5Pol-NLS;
b) nCas9-linker-T5Pol-linker-NLS;
c) T5Pol-nCas9-NLS;
d) T5Pol-linker-nCas9-linker-NLS;
e) NLS-nCas9-NLS-T5Pol-binding site-NLS-NLS;
f) NLS-nCas9-linker-NLS-linker-T5Pol-linker-MCP-linker-NLS-linker-NLS;
g) nCas9-linker-T5Pol-linker-NLS;
h) nCas9-T5Pol(N30del)-NLS;
i) nCas9-linker-T5Pol(N30del)-linker-NLS;
j) nCas9-T5Pol(DA593R)-NLS;
k) nCas9-linker-T5Pol(A593R)-linker-NLS;
l) NLS-nCas9-NLS-T5Pol-NLS-NLS;
m) NLS-nCas9-linker-NLS-linker-T5Pol-linker-NLS-linker-NLS;
n) nCas9-NLS-T5Pol;
o) nCas9-linker-NLS-linker-T5Pol;
p) NLS-nCas9-NLS-T5Pol-NLS-NLS;
q) NLS-nCas9-linker-NLS-linker-T5Pol-linker-NLS-NLS;
r) nCas9-T5Pol(K425I)-NLS;
S) nCas9-linker-T5Pol(K425I)-linker-NLS;
t) nCas9-T5Pol-NLS-heterodimerization domain, optionally wherein the heterodimerization domain comprises a coiled coil heterodimerization domain, optionally an EI domain or a KI domain; or
u) nCas9-linker-T5Pol-linker-NLS-linker-heterodimerization domain, optionally wherein the heterodimerization domain comprises a coiled coil heterodimerization domain, optionally an EI domain or KI domain, and
optionally, wherein the T5pol comprises a mutation at D164 or E166 relative to SEQ ID NO: 1116, optionally wherein the T5 pol comprises D164A and E166A mutations relative to SEQ ID NO: 1116.

Embodiment 510 is the system of any one of Embodiments 456-509, wherein the T5 pol and nCas9 are covalently linked sequentially in a polypeptide from N-terminus to C-terminus in order wherein the order is NLS-nCas9-linker-NLS-linker-T5Pol-linker-NLS-NLS; optionally, wherein the T5pol comprises a mutation at D164 or E166 relative to SEQ ID NO: 1116, optionally wherein the T5 pol comprises D164A and E166A mutations relative to SEQ ID NO: 1116.

Embodiment 511 is the system of Embodiment 456-510, wherein the T5Pol and nCas9 are covalently linked sequentially in a polypeptide from N-terminus to C-terminus, wherein the order is NLS-nCas9-linker-NLS-linker-T5Pol-linker-NLS-NLS-linker, wherein the T5Pol comprises a mutation at D164 or E166 relative to SEQ ID NO: 1116.

Embodiment 512 is the system of any one of Embodiments 456-511, wherein the polypeptide comprises a heterodimerization domain or further comprises an additional heterodimerization domain.

Embodiment 513 is the system of Embodiment 512, wherein the heterodimerization domain comprises a coiled coil heterodimerization domain.

Embodiment 514 is the system of Embodiments 512 or 513, wherein the heterodimerization domain comprises an EV, EI, KV, or KI domain.

Embodiment 515 is the system of any one of Embodiments 512-514, wherein the heterodimerization domain comprises an EI or KI domain.

Embodiment 516 is the system of any one of Embodiments 512-515, wherein the heterodimerization domain comprises SEQ ID NO: 1589, SEQ ID NO: 1593, or SEQ ID NO: 1595.

Embodiment 517 is the system of Embodiment 516, wherein the T5Pol and nCas9 are covalently linked sequentially in a polypeptide from N-terminus to C-terminus in an order of:
a) NLS-nCas9-linker-NLS-linker-T5Pol-linker-NLS-NLS-linker-EI domain; or
b) NLS-nCas9-linker-NLS-linker-T5Pol-linker-NLS-NLS-linker-KI domain;
c) wherein the T5Pol comprises a mutation at D164 or E166 relative to SEQ ID NO: 1116.

Embodiment 518 is the system of any one of Embodiments 456-517, wherein the DNA-dependent DNA polymerase and nCas9 are provided as one or more polypeptides.

Embodiment 519 is the system of any one of Embodiments 456-517, wherein the DNA-dependent DNA polymerase and nCas9 are provided as one or more nucleic acids.

Embodiment 520 is the system of any one of Embodiments 456-504, and 512-519, wherein the T5 pol or a portion thereof, is operably linked to a first split-intein and the nCas9, or a portion thereof, is operably linked to a second split-intein that is complementary to the first split intein, wherein the first split-intein and second split-intein undergo trans-splicing reaction, resulting in the formation of a single fusion polypeptide comprising, from N-terminus to C-terminus, the polk and the nCas9.

Embodiment 521 is the system of Embodiment 520, wherein the first split-intein and the second split-intein are capable of binding to each other to undergo intein-mediated protein splicing.

Embodiment 522 is the system of Embodiment 520 or 521, wherein the first split-intein is an N-split-intein and the second split-intein is a C-split intein.

Embodiment 523 is the system of any one of Embodiments 520-521, wherein the first split-intein is a C-split-intein and the second split intein is an N-split-intein.

Embodiment 524 is the system of any one of Embodiments 520-523, wherein the first split-intein and second split-intein are complementary inteins selected from Cfa, M86, Npu, NpuSsp, gp41-1, gp41-8, NrdJ-1, IMPDH-1, SspDnaX, SspGyrB, Cth-Ter, NrdA-2, Mja-KlbA, RBS1, TE3S11, and Rma.

Embodiment 525 is the system of any one of Embodiments 520-524, wherein the first split-intein and second split-intein are Cfa-inteins.

Embodiment 526 is the system of any one of Embodiments 525, wherein the first split-intein is a Cfa C-split-intein and the second split-intein is a Cfa N-split-intein.

Embodiment 527 is the system of any one of Embodiments 525, wherein the Cfa C-split-intein comprises an amino acid sequence having at least 90% identity to SEQ ID NO: 1746 and the Cfa N-split-intein comprises an amino acid sequence having at least 90% identity to SEQ ID NO: 1744.

Embodiment 528 is the system of any one of Embodiments 525, wherein the Cfa C-split-intein comprises the amino acid sequence of SEQ ID NO: 1746 and the Cfa N-split-intein comprises the amino acid sequence of SEQ ID NO: 1744.

Embodiment 529 is the system of any one of Embodiments 520-528, wherein the T5 pol is operably linked to the first split-intein by a first peptide linker.

Embodiment 530 is the system of any one of Embodiments 529, wherein the first peptide linker comprises 30 amino acids, optionally wherein the first peptide linker comprises an amino acids sequence at least 90%, 95%, 98%, or 100% identical to SEQ ID NO: 1829.

Embodiment 531 is the system of any one of Embodiments 520-530, wherein the nCas9 is operably linked to the second split-intein by a second peptide linker, optionally wherein the second peptide linker is SGGS (SEQ ID NO: 1738).

Embodiment 532 is the system of any one of Embodiments 520-531, wherein the T5 pol is operably linked to a heterologous nuclear localization signal (NLS).

Embodiment 533 is the system of any one of Embodiments 520-532, wherein the nCas9 is operably linked to a heterologous nuclear localization signal (NLS).

Embodiment 534 is the system of any one of Embodiments 532 or 533, wherein the heterologous nuclear localization signal is selected from an SV40 NLS, SV40-c-Myc NLS, and nucleoplasmin NLS.

Embodiment 535 is the system of any one of Embodiments 456-519, wherein the T5 pol and the nCas9 are covalently linked, wherein the T5 pol and nCas9 are operably linked to at least one chromatin remodeler.

Embodiment 536 is the system of Embodiment 535, wherein the T5 pol, nCas9, and chromatin remodeler are covalently linked sequentially in a polypeptide from N-terminus to C-terminus in an order selected from:
a) chromatin remodeler-nCas9-T5 pol;
b) nCas9-chromatin remodeler-T5 pol; or
c) nCas9-T5 pol-chromatin remodeler.

Embodiment 537 is the system of Embodiment 535 or 536, further comprising a nuclear localization signal (NLS).

Embodiment 538 is the system of any one of Embodiments 535-537, wherein the T5 pol, nCas9, and chromatin remodeler are covalently linked sequentially in a polypeptide from N-terminus to C-terminus in an order selected from:
a) NLS-chromatin remodeler-nCas9-T5 pol-NLS;
b) NLS-chromatin remodeler-nCas9-NLS-T5 pol-NLS;
c) NLS-nCas9-chromatin remodeler-T5 pol-NLS; or
d) nCas9-T5 pol-chromatin remodeler-NLS.

Embodiment 539 is the system of any one of Embodiments 535-538, further comprising one or more linkers between the NLS, nCas9, T5 pol, and the chromatin remodeler.

Embodiment 540 is the system of Embodiment 535-539, wherein the at least one chromatin remodeler is selected from a polypeptide comprising HMGB1, HMGN1, HMGN2, HMGN3a, HMGN3b, HMGN4, HMGN5, histone H1.0, histone H1.2, CHD1, ISWI, DNA helicase, TOP1, herpesvirus 8 LANA, CMV IE1 or a domain thereof.

Embodiment 541 is the system of any one of Embodiments 535-540, wherein the at least one chromatin remodeler comprises an HMGB1 polypeptide.

Embodiment 542 is the system of Embodiment 541, wherein the HMGB1 polypeptide comprises a sequence at least 90%, 91%, 92%, 93%, 94%, 95%, 96%, 97%, 98%, 99%, or 100% identical to SEQ ID NO: 1783.

Embodiment 543 is the system of Embodiment 541 or 542, wherein the HMGB1 polypeptide comprises an HMGB1 polypeptide, wherein the HMGB1 polypeptide comprises an HMGB1 Box B domain and at least 7 contiguous amino acid residues C-terminal to the HMGB1 Box B domain, and wherein the HMGB1 polypeptide lacks all or part of an acidic tail domain.

Embodiment 544 is the system of any one of Embodiments 541-543, wherein the HMGB1 polypeptide comprises a cryptic nuclear localization signal (NLS).

Embodiment 545 is the system of Embodiment 544, wherein the cryptic NLS comprises the sequence of EKSKKKK (SEQ ID NO: 1787).

Embodiment 546 is the system of any one of Embodiments 541-545, wherein the HMGB1 polypeptide comprises a receptor for advanced glycation end-products (RAGE) binding domain.

Embodiment 547 is the system of Embodiment 546, wherein the RAGE binding domain comprises the sequence of SEQ ID NO: 1803.

Embodiment 548 is the system of any one of Embodiments 541-547, wherein the HMGB1 polypeptide comprises 7-20 amino acid residues C-terminal to the HMGB1 Box B domain.

Embodiment 549 is the system of any one of Embodiments 541-548, wherein the HMGB1 polypeptide comprises the sequence of SEQ ID NO: 1804.

Embodiment 550 is the system of any one of Embodiments 541-549, wherein the HMGB1 polypeptide lacks a sequence at least 50%, 60%, 70%, 80%, 90%, 94%, or 97% identical to SEQ ID NO: 1788 or lacks the sequence of SEQ ID NO: 1788.

Embodiment 551 is the system of any one of Embodiments 541-550, wherein the HMGB1 polypeptide lacks transcriptional stimulatory function.

Embodiment 552 is the system of any one of Embodiments 541-551, wherein the HMGB1 polypeptide comprises a cryptic NLS that is located C-terminal to the HMGB1 Box B domain.

Embodiment 553 is the system of any one of Embodiments 541-552, wherein the at least 7-20 contiguous amino acid residues C-terminal end of the HMGB1 Box B domain comprise the cryptic NLS.

Embodiment 554 is the system of any one of Embodiments 541-553, wherein the cryptic NLS immediately follows the C-terminal end of the HMGB1 Box B domain.

Embodiment 555 is the system of any one of Embodiments 541-554, wherein the HMGB1 polypeptide lacks an HMGB1 Box A domain.

Embodiment 556 is the system of any one of Embodiments 541-555, wherein the HMGB1 polypeptide comprises two HMGB1 Box B domains.

Embodiment 557 is the system of any one of Embodiments 541-556, wherein the cryptic NLS is located C-terminal to the C-terminal end of the two HMGB1 Box B domains.

Embodiment 558 is the system of any one of Embodiments 541-554 and 556-557, wherein the HMGB1 polypeptide comprises an HMGB1 Box A domain.

Embodiment 559 is the system of any one of Embodiments 541-554 and 556-558, wherein the Box B domain is located C-terminal to the HMGB1 Box A domain.

Embodiment 560 is the system of any one of Embodiments 541-554 and 556-559, wherein the HMGB1 polypeptide comprises, from N-terminus to C-terminus:
a) HMGB1 Box A domain-HMGB1 Box B domain-cryptic NLS;
b) HMGB1 Box B domain-HMGB1 Box B domain-cryptic NLS; or
c) HMGB1 Box B domain-cryptic NLS.

Embodiment 561 is the system of any one of Embodiments 541-560, wherein the HMGB1 polypeptide comprises at least 90% sequence identity to SEQ ID NO: 1783-1792 and 1803-1804 or the amino acid sequence of SEQ ID NO: 1783-1792 and 1803-1804.

Embodiment 562 is the system of any one of Embodiments 512-516 and 518-561, wherein the T5 pol and the nCas9 are not covalently linked, and the T5 pol is provided with a 3′ extension-recruiting domain, wherein, the T5 pol and the 3′ extension-recruiting domain are covalently linked sequentially in a polypeptide from N-terminus to C-terminus in an order selected from:
a) 3′ extension-recruiting domain-T5Pol(D164A,E166A)-NLS; or
b) 3′ extension-recruiting domain-linker-T5Pol(D164A,E166A)-linker-NLS.

Embodiment 563 is the system of any one of Embodiments 509-562, wherein the polypeptide comprises a peptide linker or further comprises an additional peptide linker.

Embodiment 564 is the system of Embodiment 562 or 563, wherein the polypeptide comprises a heterodimerization domain or further comprises an additional heterodimerization domain.

Embodiment 565 is the system of Embodiment 564, wherein the heterodimerization domain comprises a coiled coil heterodimerization domain.

Embodiment 566 is the system of Embodiment 564 or 565, wherein the heterodimerization domain comprises an EV, EI, KV, or KI domain.

Embodiment 567 is the system of any one of Embodiments 564-566, wherein the heterodimerization domain comprises an EI or KI domain.

Embodiment 568 is the system of any one of Embodiments 564-567, wherein the heterodimerization domain comprises SEQ ID NO: 1589, SEQ ID NO: 1593, or SEQ ID NO: 1595.

Embodiment 569 is the system of any one of Embodiments 504-568, wherein the NLS is selected from a c-Myc NLS, an SV40 NLS, a nucleoplasmin NLS, or a snurportin-1 importin-β NLS.

Embodiment 570 is the system of any one of Embodiment 569, wherein each NLS is independently selected from an SV40 NLS, a nucleoplasmin NLS, or a c-Myc NLS.

Embodiment 571 is the system of Embodiment 569 or 570, wherein the T5Pol and nCas9 are covalently linked sequentially in a polypeptide from N-terminus to C-terminus, wherein the order is:
a) nucleoplasmin NLS-nCas9-linker-SV40 NLS-linker-T5Pol-linker-SV40 NLS-c-Myc NLS;
b) nucleoplasmin NLS-nCas9-linker-SV40 NLS-linker-T5Pol-linker-SV40 NLS-c-Myc NLS-EI domain; or
c) nucleoplasmin NLS-nCas9-linker-SV40 NLS-linker-T5Pol-linker-SV40 NLS-c-Myc NLS-KI domain;
wherein the T5Pol comprises a mutation at D164 or E166 relative to SEQ ID NO: 1116.

Embodiment 572 is the system of any one of Embodiments 504-519 and 535-571, wherein the polypeptide comprises a sequence that has at least 90%, 91%, 92%, 93%, 94%, 95%, 96%, 97%, 98%, 99%, or 100% identity to any one of SEQ ID NO: 1124, 1127, 1129, 1132, 1134, 1137, 1140, 1142, 1145, 1147, 1150, 1152, 1155, 1157, 1160, 1162, 1165, 1167, 1170, 1172, 1216, 1219, 1224, 1254, and 1259, 1276, 1303, 1307, 1376, 1450, 1932, 1935, and 1938.

Embodiment 573 is the system of any one of Embodiments 504-519 and 535-572, wherein the polypeptide comprises a sequence that has at least 90%, 91%, 92%, 93%, 94%, 95%, 96%, 97%, 98%, 99%, or 100% identity to any one of SEQ ID NO: 1124, 1129, 1134, 1137, 1142, 1147, 1152, 1157, 1162, 1167, 1172, 1219, 1224, 1254, and 1259, 1276, 1307, 1932, 1935, and 1938.

Embodiment 574 is the system of any one of Embodiments 504-519 and 535-573, wherein the polypeptide comprises a sequence selected from any one of SEQ ID NO: 1124, 1127, 1129, 1132, 1134, 1137, 1140, 1142, 1145, 1147, 1150, 1152, 1155, 1157, 1160, 1162, 1165, 1167, 1170, 1172, 1216, 1219, 1224, 1254, and 1259, 1276, 1303, 1307, 1376, 1450, 1932, 1935, and 1938.

Embodiment 575 is the system of any one of Embodiments 504-519 and 535-574, wherein the polypeptide comprises a sequence selected from any one of SEQ ID NO: 1124, 1129, 1134, 1137, 1142, 1147, 1152, 1157, 1162, 1167, 1172, 1219, 1224, 1254, and 1259, 1276, 1307, 1932, 1935, and 1938.

Embodiment 576 is the system of any one of Embodiments 504-519 and 535-575, wherein the polypeptide is encoded by a nucleotide sequence that has at least 90%, 91%, 92%, 93%, 94%, 95%, 96%, 97%, 98%, 99%, or 100% identity to any one of SEQ ID NOs: 1123, 1126, 1128, 1131, 1133, 1136, 1139, 1141, 1144, 1146, 1149, 1151, 1154, 1156, 1159, 1161, 1164, 1166, 1169, 1171, 1215, 1218, 1223, 1253, and 1258, 1275, 1302, 1306, 1375, 1449, 1931, 1934, and 1937.

Embodiment 577 is the system of any one of Embodiments 504-519 and 535-576, wherein the polypeptide is encoded by a nucleotide sequence that has at least 90%, 91%, 92%, 93%, 94%, 95%, 96%, 97%, 98%, 99%, or 100% identity to any one of SEQ ID NOs: 1123, 1128, 1133, 1136, 1141, 1146, 1151, 1156, 1161, 1166, 1171, 1218, 1223, 1253, and 1258, 1275, 1306, 1931, 1934, and 1937.

Embodiment 578 is the system of any one of Embodiments 562-571, wherein the polypeptide comprises a sequence that has at least 90%, 91%, 92%, 93%, 94%, 95%, 96%, 97%, 98%, 99%, or 100% identity to any one of SEQ ID NO: 1175, 1177, 1180, and 1182.

Embodiment 579 is the system of any one of Embodiments 562-571 or 578, wherein the polypeptide comprises a sequence that has at least 90%, 91%, 92%, 93%, 94%, 95%, 96%, 97%, 98%, 99%, or 100% identity to SEQ ID NO: 1177 or 1182.

Embodiment 580 is the system of any one of Embodiments 562-571 or 578 or 579, wherein the polypeptide is encoded by a nucleotide sequence that has at least 90%, 91%, 92%, 93%, 94%, 95%, 96%, 97%, 98%, 99%, or 100% identity to any one of SEQ ID NOs: 1174, 1176, 1179, and 1181.

Embodiment 581 is the system of any one of Embodiments 562-571 or 578-580, wherein the polypeptide is encoded by a nucleotide sequence that has at least 90%, 91%, 92%, 93%, 94%, 95%, 96%, 97%, 98%, 99%, or 100% identity to SEQ ID NOs: 1176 or 1181.

Embodiment 582 is a fusion polypeptide comprising a polK comprising GE411-412RV mutations relative to SEQ ID NO: 1021, a SpyCas9 nickase (nCas9), and a heterologous nuclear localization signal (NLS) or a nucleic acid encoding the fusion polypeptide.

Embodiment 583 is the fusion polypeptide or nucleic acid of Embodiment 582, wherein the polK comprises (1) an amino acid sequence that has at least 90%, 91%, 92%, 93%, 94%, 95%, 96%, 97%, 98%, or 99% identity to amino acid residues 19-526 of SEQ ID NO: 1021; or (2) an amino acid sequence that has at least 90%, 91%, 92%, 93%, 94%, 95%, 96%, 97%, 98%, 99%, or 100% identity to SEQ ID NO: 1021; or (3) an amino acid sequence of residues 19-526 of SEQ ID NO: 1021.

Embodiment 584 is the fusion polypeptide or nucleic acid of any one of Embodiments 582 or 583, wherein the polK is encoded by a nucleotide sequence that has at least 90%, 91%, 92%, 93%, 94%, 95%, 96%, 97%, 98%, 99%, or 100% identity to any one of SEQ ID NOs: 1020 or 1205.

Embodiment 585 is the fusion polypeptide or nucleic acid of any one of Embodiments 582-584, wherein the nCas9 comprises an amino acid sequence that has at least 90%, 91%, 92%, 93%, 94%, 95%, 96%, 97%, 98%, 99%, or 100% identity to any one of SEQ ID NOs: 972, 974, 976, 982, 984, 986, 988, 990, 992, 994, 996, 998, 1000, and 1014.

Embodiment 586 is the fusion polypeptide or nucleic acid of any one of Embodiments 582-585, wherein the nCas9 comprises an amino acid sequence of any one of SEQ ID NOs: 972, 974, 976, 982, 984, 986, 988, 990, 992, 994, 996, 998, 1000, and 1014.

Embodiment 587 is the fusion polypeptide or nucleic acid of any one of Embodiments 582-586, wherein the nCas9 is encoded by a nucleotide sequence that has at least 90%, 91%, 92%, 93%, 94%, 95%, 96%, 97%, 98%, 99%, or 100% identity to any one of SEQ ID NOs: 971, 973, 975, 981, 983, 985, 987, 989, 991, 993, 995, 997, 999, and 1013.

Embodiment 588 is the fusion polypeptide or nucleic acid of any one of Embodiments 582-587, wherein the polK and nCas9 are in a sequence from N-terminus to C-terminus, polk-nCas9.

Embodiment 589 is the fusion polypeptide or nucleic acid of any one of Embodiments 582-587, wherein the polk and nCas9 are in a sequence from N-terminus to C-terminus, nCas9-polK.

Embodiment 590 is the fusion polypeptide or nucleic acid of any one of Embodiments 582-589, further comprising a peptide linker or a heterologous nuclear localization signal (NLS).

Embodiment 591 is the fusion polypeptide or nucleic acid of any one of Embodiments 582-590, wherein the polk and nCas9 are covalently linked sequence from N-terminus to C-terminus selected from:
a) NLS-nCas9-NLS-polK;
b) NLS-nCas9-linker-NLS-linker-polK;
c) polK-NLS-nCas9-NLS;
d) polK-linker-NLS-linker-nCas9-linker-NLS;
e) NLS-nCas9-NLS-polK(GE411-412RV);
f) NLS-nCas9-linker-NLS-polK(GE411-412RV);
g) NLS-nCas9-NLS-polK(19-526);
h) NLS-nCas9-linker-NLS-linker-polK(19-526);
i) polK(19-526)-NLS-nCas9-NLS;
j) polK(19-526)-Linker-NLS-linker-nCas9-linker-NLS;
k) polK(GE411-412RV)-NLS-NLS-nCas9;
l) polK(GE411-412RV)-linker-NLS-linker-NLS-nCas9;
m) NLS-nCas9-NLS-polK(19-526, GE411-412RV);
n) NLS-nCas9-linker-NLS-linker-polK(19-526, GE411-412RV);
o) NLS-nCas9-NLS-polK-NLS;
p) NLS-nCas9-linker-NLS-linker-polK-linker-NLS;
q) NLS-nCas9-NLS-polK(1-526, GE411-412RV)-NLS;
r) NLS-nCas9-linker-NLS-linker-polK(1-526, GE411-412RV)-linker-NLS;
s) nCas9-polK(1-526);
t) polK(1-526, GE411-412RV)-nCas9-NLS-heterodimerization domain, optionally wherein the heterodimerization domain comprises a coiled coil heterodimerization domain, optionally an EI domain or a KI domain;
u) polK(1-526, GE411-412RV)-linker-NLS-nCas9-linker-NLS-heterodimerization domain, optionally wherein the heterodimerization domain comprises a coiled coil heterodimerization domain, optionally an EI domain or KI domain;
optionally wherein the order is NLS-nCas9-linker-NLS-linker-polK(1-526, GE411-412RV)-linker-NLS, optionally further comprising a heterodimerization domain comprising a coiled coil heterodimerization domain, optionally an EI domain or KI domain.

Embodiment 592 is the fusion polypeptide of Embodiment 591, wherein polk and nCas9 are covalently linked sequentially in a polypeptide from N-terminus to C-terminus, wherein the order is:
a) NLS-nCas9-linker-NLS-linker-polK(1-526, GE411-412RV)-linker-NLS-linker-EI domain; or
b) NLS-nCas9-linker-NLS-linker-polK(1-526, GE411-412RV)-linker-NLS-linker-KI domain.

Embodiment 593 is the fusion polypeptide or nucleic acid of any one of Embodiments 582-592, wherein the polypeptide comprises a peptide linker or further comprises an additional peptide linker.

Embodiment 594 is the fusion polypeptide of any one of Embodiments 582-591 or 593, wherein the polypeptide comprises a heterodimerization domain or further comprises an additional heterodimerization domain.

Embodiment 595 is the fusion polypeptide of Embodiment 594, wherein the heterodimerization domain comprises a coiled coil heterodimerization domain.

Embodiment 596 is the fusion polypeptide of Embodiment 594 or 595, wherein the heterodimerization domain comprises an EV, EI, KV, or KI domain.

Embodiment 597 is the fusion polypeptide of any one of Embodiments 594-596, wherein the heterodimerization domain comprises an EI or KI domain.

Embodiment 598 is the fusion polypeptide of any one of Embodiments 594-597, wherein the heterodimerization domain comprises SEQ ID NO: 1589, SEQ ID NO: 1593, or SEQ ID NO: 1595.

Embodiment 599 is the fusion polypeptide or nucleic acid of any one of Embodiments 590-598, wherein the NLS is selected from a c-Myc NLS, an SV40 NLS, a nucleoplasmin NLS, or a snurportin-1 importin-β NLS, optionally wherein the NLS is an SV40 NLS.

Embodiment 600 is the fusion polypeptide of any one of Embodiments 582-599, wherein polK and nCas9 are covalently linked sequentially in a polypeptide from N-terminus to C-terminus, wherein the order is:
a) SV40 NLS-nCas9-linker-SV40 NLS-linker-polK(1-526, GE411-412RV)-linker-SV40NLS;
b) SV40 NLS-nCas9-linker-SV40 NLS-linker-polK(1-526, GE411-412RV)-linker-SV40 NLS-linker-EI domain; or
c) SV40 NLS-nCas9-linker-SV40 NLS-linker-polK(1-526, GE411-412RV)-linker-SV40 NLS-linker-KI domain.

Embodiment 601 is the fusion polypeptide or nucleic acid of any one of Embodiments 582-600, wherein the polypeptide comprises a sequence selected from any one of SEQ ID NOs: 1024, 1027, 1030, 1036, 1039, 1042, 1045, 1048, 1053, 1056, 1059, 1074, 1200, 1202, 1229, 1234, and 1268, 1300, 1373, 1388, 1447, 1453, 1923, 1926, and 1929.

Embodiment 602 is the fusion polypeptide or nucleic acid of any one of Embodiments 582-601, wherein the polypeptide is encoded by a nucleic acid comprising a nucleotide sequence that has at least 90%, 91%, 92%, 93%, 94%, 95%, 96%, 97%, 98%, 99%, or 100% identity to any one of SEQ ID NOs: 1023, 1026, 1029, 1035, 1038, 1041, 1044, 1047, 1052, 1055, 1058, 1073, 1199, 1201, 1228, 1233, and 1267, and 1299, 1372, 1387, 1446, 1452, 1922, 1925, and 1928.

Embodiment 603 is a fusion polypeptide comprising a T5 pol comprising D164A and E166A mutations relative to SEQ ID NO: 1116, a SpyCas9 nickase (nCas9), a heterologous nuclear localization signal (NLS), and optionally a heterodimerization domain, or a nucleic acid encoding the fusion polypeptide thereof.

Embodiment 604 is the fusion polypeptide or nucleic acid of Embodiment 603, wherein the T5 pol comprises an amino acid sequence that has at least 90%, 91%, 92%, 93%, 94%, 95%, 96%, 97%, 98%, 99%, or 100% identity to any one of SEQ ID NO: 1116 or 1121, optionally further wherein the T5 pol comprises a heterodimerization domain.

Embodiment 605 is the fusion polypeptide or nucleic acid of Embodiment 603 or 604, wherein the T5 pol comprises an N-terminal deletion of 30 amino acids relative to SEQ ID NO: 1116.

Embodiment 606 is the fusion polypeptide or nucleic acid of any one of Embodiments 603-605, wherein the T5 pol is encoded by a nucleotide sequence that has at least 90%, 91%, 92%, 93%, 94%, 95%, 96%, 97%, 98%, 99%, or 100% identity to any one of SEQ ID NOs: 1115 or 1120.

Embodiment 607 is the fusion polypeptide or nucleic acid of any one of Embodiments 603-606, wherein the nCas9 comprises an amino acid sequence that has at least 90%, 91%, 92%, 93%, 94%, 95%, 96%, 97%, 98%, 99%, or 100% identity to any one of SEQ ID NOs: 972, 974, 976, 982, 984, 986, 988, 990, 992, 994, 996, 998, 1000, and 1014, optionally wherein the nCas9 protein includes or lacks an N-terminal methionine relative to any one of SEQ ID NOs: 972, 974, 976, 982, 984, 986, 988, 990, 992, 994, 996, 998, 1000, and 1014.

Embodiment 608 is the fusion polypeptide or nucleic acid of any one of Embodiments 603-607, wherein the nCas9 comprises an amino acid sequence of any one of SEQ ID NOs: 972, 974, 976, 982, 984, 986, 988, 990, 992, 994, 996, 998, 1000, and 1014, optionally wherein the nCas9 protein includes or lacks an N-terminal methionine relative to any one of SEQ ID NOs: 972, 974, 976, 982, 984, 986, 988, 990, 992, 994, 996, 998, 1000, and 1014.

Embodiment 609 is the fusion polypeptide or nucleic acid of any one of Embodiments 603-608, wherein the nCas9 is encoded by a nucleotide sequence that has at least 90%, 91%, 92%, 93%, 94%, 95%, 96%, 97%, 98%, 99%, or 100% identity to any one of SEQ ID NOs: 971, 973, 975, 981, 983, 985, 987, 989, 991, 993, 995, 997, 999, and 1013, optionally wherein the nCas9 protein includes or lacks a start codon or stop codon relative to any one of SEQ ID NOs: 971, 973, 975, 981, 983, 985, 987, 989, 991, 993, 995, 997, 999, and 1013.

Embodiment 610 is the fusion polypeptide or nucleic acid of any one of Embodiments 603-609, wherein the T5 pol and the nCas9 are in a sequence from N-terminus to C-terminus, nCas9-T5 pol.

Embodiment 611 is the fusion polypeptide or nucleic acid of any one of Embodiments 603-610, further comprising a peptide linker.

Embodiment 612 is the fusion polypeptide or nucleic acid of any one of Embodiments 603-611, further comprising a heterodimerization domain.

Embodiment 613 is the fusion polypeptide or nucleic acid of any one of Embodiments 603-612, wherein the T5Pol and nCas9 are covalently linked in sequence from N-terminus to C-terminus selected from:
a) nCas9-T5Pol-NLS
b) nCas9-linker-T5Pol-linker-NLS;
c) T5Pol-nCas9-NLS;
d) T5Pol-linker-nCas9-linker-NLS;
e) NLS-nCas9-NLS-T5Pol-binding site-NLS-NLS;
f) NLS-nCas9-linker-NLS-linker-T5Pol-linker-MCP-linker-NLS-linker-NLS;
g) nCas9-linker-T5Pol-linker-NLS;
h) nCas9-T5Pol(N30del,)-NLS;
i) nCas9-linker-T5Pol(N30del)-linker-NLS;
j) nCas9-T5Pol(A593R)-NLS;
k) nCas9-linker-T5Pol(DA593R)-linker-NLS;
l) NLS-nCas9-NLS-T5Pol-NLS-NLS;
m) NLS-nCas9-linker-NLS-linker-T5Pol-linker-NLS-linker-NLS;
n) nCas9-NLS-T5Pol;
o) nCas9-linker-NLS-linker-T5Pol;
p) NLS-nCas9-NLS-T5Pol-NLS-NLS;
q) NLS-nCas9-linker-NLS-linker-T5Pol-linker-NLS-NLS;
r) nCas9-T5Pol(K425I)-NLS; or
s) nCas9-linker-T5Pol(K425I)-linker-NLS;
t) nCas9-T5Pol-NLS-heterodimerization domain, optionally wherein the heterodimerization domain comprises a coiled coil heterodimerization domain, optionally an EI domain or a KI domain; or
u) nCas9-linker-T5Pol-linker-NLS-linker-heterodimerization domain, optionally wherein the heterodimerization domain comprises a coiled coil heterodimerization domain, optionally an EI domain or a KI domain,
optionally, wherein the T5pol comprises a mutation at D164 or E166 relative to SEQ ID NO: 1116, optionally wherein the T5 pol comprises D164A and E166A mutations relative to SEQ ID NO: 1116.

Embodiment 614 is the fusion polypeptide or nucleic acid of any one of Embodiments 603-613, wherein the T5 pol and nCas9 are covalently linked sequentially in a polypeptide from N-terminus to C-terminus in order wherein the order is NLS-nCas9-linker-NLS-linker-T5Pol-linker-NLS-NLS; optionally, wherein the T5pol comprises a mutation at D164 or E166 relative to SEQ ID NO: 1116, optionally wherein the T5 pol comprises D164A and E166A mutations relative to SEQ ID NO: 1116.

Embodiment 615 is the fusion polypeptide of any one of Embodiments 603-614, wherein the polypeptide comprises a heterodimerization domain or further comprises an additional heterodimerization domain.

Embodiment 616 is the fusion polypeptide of Embodiment 615, wherein the heterodimerization domain comprises a coiled coil heterodimerization domain.

Embodiment 617 is the fusion polypeptide of Embodiment 615 or 616, wherein the heterodimerization domain comprises an EV, EI, KV, or KI domain.

Embodiment 618 is the fusion polypeptide of any one of Embodiments 615-617, wherein the heterodimerization domain comprises an EI or KI domain.

Embodiment 619 is the fusion polypeptide of any one of Embodiments 615-618, wherein the heterodimerization domain comprises SEQ ID NO: 1589, SEQ ID NO: 1593, or SEQ ID NO: 1595.

Embodiment 620 is the fusion polypeptide or nucleic acid of any one of Embodiments 613-619, wherein the polypeptide comprises a linker or further comprises an additional linker.

Embodiment 621 is the fusion polypeptide or nucleic acid of one of Embodiments 611-620, wherein, the linker comprises an amino acid sequence of any one of SEQ ID NOs: 1596-1665, 1735, 1738, 1815, 1825, 1827, and 1829-1830.

Embodiment 622 is the fusion polypeptide or nucleic acid of any one of Embodiments 603-621, wherein the NLS is selected from a c-Myc NLS, an SV40 NLS, a nucleoplasmin NLS, or a snurportin-1 importin-β NLS, optionally wherein the NLS is selected from c-Myc NLS, an SV40 NLS, or a nucleoplasmin NLS.

Embodiment 623 is the fusion polypeptide of Embodiment 622, wherein the T5Pol and nCas9 are covalently linked sequentially in a polypeptide from N-terminus to C-terminus, wherein the order is:
a) nucleoplasmin NLS-nCas9-linker-SV40 NLS-linker-T5Pol-linker-SV40 NLS-c-Myc NLS;
b) nucleoplasmin NLS-nCas9-linker-SV40 NLS-linker-T5Pol-linker-SV40 NLS-c-Myc NLS-EI domain; or
c) nucleoplasmin NLS-nCas9-linker-SV40 NLS-linker-T5Pol-linker-SV40 NLS-c-Myc NLS-KI domain;
d) wherein the T5Pol comprises a mutation at D164 or E166 relative to SEQ ID NO: 1116.

Embodiment 624 is the fusion polypeptide or nucleic acid of any one of Embodiments 603-623, wherein the polypeptide comprises a sequence selected from any one of SEQ ID NO: 1124, 1127, 1129, 1132, 1134, 1137, 1140, 1142, 1145, 1147, 1150, 1152, 1155, 1157, 1160, 1162, 1165, 1167, 1170, 1172, 1216, 1219, 1224, 1254, and 1259, 1276, 1303, 1307, 1376, 1450, 1932, 1935, and 1938.

Embodiment 625 is the fusion polypeptide or nucleic acid of any one of Embodiments 603-624, wherein the polypeptide comprises a sequence selected from any one of SEQ ID NO: 1124, 1129, 1134, 1137, 1142, 1147, 1152, 1157, 1162, 1167, 1172, 1219, 1224, 1254, and 1259, 1276, 1307, 1932, 1935, and 1938.

Embodiment 626 is the fusion polypeptide or nucleic acid of any one of Embodiments 603-625, wherein the polypeptide is encoded by a nucleotide sequence that has at least 90%, 91%, 92%, 93%, 94%, 95%, 96%, 97%, 98%, 99%, or 100% identity to any one of SEQ ID NOs: 1123, 1126, 1128, 1131, 1133, 1136, 1139, 1141, 1144, 1146, 1149, 1151, 1154, 1156, 1159, 1161, 1164, 1166, 1169, 1171, 1215, 1218, 1223, 1253, and 1258, 1275, 1302, 1306, 1375, 1449, 1931, 1934, and 1937.

Embodiment 627 is the fusion polypeptide or nucleic acid of any one of Embodiments 603-626, wherein the polypeptide is encoded by a nucleotide sequence that has at least 90%, 91%, 92%, 93%, 94%, 95%, 96%, 97%, 98%, 99%, or 100% identity to any one of SEQ ID NOs: 1123, 1128, 1133, 1136, 1141, 1146, 1151, 1156, 1161, 1166, 1171, 1218, 1223, 1253, and 1258, 1275, 1306, 1931, 1934, and 1937.

Embodiment 628 is a system comprising (i) a fusion polypeptide comprising a DNA-dependent DNA polymerase and a SpyCas9 nickase (nCas9), a nucleic acid encoding the fusion polypeptide, (ii) a template guide RNA (tgRNA), and (iii) an enhancer of template-based genome editing, wherein:
a) the fusion polypeptide comprising the DNA-dependent DNA polymerase and the nCas9 is a fusion polypeptide of any one of Embodiments 45-65, 82-111, or 582-627;
b) the tgRNA comprises a first polynucleotide and a second polynucleotide, wherein:
   1) the first polynucleotide comprises a SpyCas9 guide RNA comprising, from 5′ to 3′:
      1. a spacer that is complementary to a target region of a target strand of a DNA duplex target nucleic acid; and
      2. a scaffold; and
   2) the second polynucleotide is operably linked to the first polynucleotide and comprises, from 5′ to 3′:
      1. a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides;
      2. a DNA-dependent DNA polymerase recruiting sequence (DRS) that is 6-20 nucleotides in length, optionally 8-17 or 10-14 nucleotides in length and having a 3′ end and a 5′ end; wherein the 5′ end of the DRS is covalently attached to the 3′ end of the template sequence, and wherein the DRS comprises a sequence that is complementary to at least 6 consecutive nucleotides of a DRS-complementary region of the non-target strand of the DNA duplex target nucleic acid; and
c) the enhancer of template-based genome editing.

Embodiment 629 is A system comprising (i) a fusion polypeptide comprising a DNA-dependent DNA polymerase and a SpyCas9 nickase (nCas9), a nucleic acid encoding the fusion polypeptide, (ii) a template guide RNA (tgRNA), and (iii) at least two enhancers of template-based genome editing, wherein:
a) the fusion polypeptide comprising the DNA-dependent DNA polymerase and the nCas9 is a fusion polypeptide of any one of Embodiments 45-65, 82-111, or 582-627;
b) the tgRNA comprises a first polynucleotide and a second polynucleotide, wherein:
   3) the first polynucleotide comprises a SpyCas9 guide RNA comprising, from 5′ to 3′:
      1. a spacer that is complementary to a target region of a target strand of a DNA duplex target nucleic acid; and
      2. a scaffold; and
   4) the second polynucleotide is operably linked to the first polynucleotide and comprises, from 5′ to 3′:
      1. a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides;
      2. a DNA-dependent DNA polymerase recruiting sequence (DRS) that is 6-20 nucleotides in length, optionally 8-17 or 10-14 nucleotides in length and having a 3′ end and a 5′ end; wherein the 5′ end of the DRS is covalently attached to the 3′ end of the template sequence, and wherein the DRS comprises a sequence that is complementary to at least 6 consecutive nucleotides of a DRS-complementary region of the non-target strand of the DNA duplex target nucleic acid; and
c) the at least two enhancers of template-based genome editing.

Embodiment 630 is the system of Embodiment 628 or 629, wherein the enhancer of template-based genome editing comprises a nucleic acid encoding the enhancer of template-based genome editing or an enhancer of template-based genome editing polypeptide.

Embodiment 631 is the system of any one of Embodiments 628-630, wherein the enhancer of template-based genome editing is an inhibitor of deoxynucleotide triphosphohydrolase.

Embodiment 632 is the system of Embodiment 631, wherein the inhibitor of deoxynucleotide triphosphohydrolase comprises a Vpx, a BGLF4, an M97, or an ORF36.

Embodiment 633 is the system of Embodiment 632, wherein the enhancer of template-based genome editing is selected from:
a) a Vpx protein comprising an amino acid sequence that has at least 90%, 91%, 92%, 93%, 94%, 95%, 96%, 97%, 98%, 99%, or 100% identity to any one of SEQ ID NOs: 1464, 1469, and 1503-1505, optionally wherein the Vpx protein includes or lacks an N-terminal methionine relative to any one of SEQ ID NO: 1464, 1469, and 1503-1505, or a nucleic acid encoding the amino acid sequence;
b) a BGLF4 protein comprising an amino acid sequence that has at least 90%, 91%, 92%, 93%, 94%, 95%, 96%, 97%, 98%, 99%, or 100% identity to SEQ ID NO: 1517 or 1518, optionally wherein the BGLF4 protein includes or lacks an N-terminal methionine relative to SEQ ID NO: 1517 or 1518, or a nucleic acid encoding the amino acid sequence; or
c) an M97 protein comprising an amino acid sequence that has at least 90%, 91%, 92%, 93%, 94%, 95%, 96%, 97%, 98%, 99%, or 100% identity to SEQ ID NO: 1500, optionally wherein the M97 protein includes or lacks an N-terminal methionine relative to SEQ ID NO: 1500, or a nucleic acid encoding the amino acid sequence; or
d) a KSHV ORF36 protein comprising an amino acid sequence that has at least 90%, 91%, 92%, 93%, 94%, 95%, 96%, 97%, 98%, 99%, or 100% identity to SEQ ID NO: 1576, optionally wherein the KSHV ORF36 protein includes or lacks an N-terminal methionine relative to SEQ ID NO: 1576, or a nucleic acid encoding the amino acid sequence.

Embodiment 634 is the system of Embodiment 632 or 633, wherein the inhibitor of deoxynucleotide triphosphohydrolase is encoded by a nucleotide sequence that has at least 90%, 91%, 92%, 93%, 94%, 95%, 96%, 97%, 98%, 99%, or 100% identity to any one of SEQ ID NOs: 1462, 1463, 1468, 1470, 1499, 1501, 1502, 1521-1523, 1574-1575, and 1585, and 3530.

Embodiment 635 is the system of Embodiment 628 or 630, wherein the enhancer of template-based genome editing is a DNA repair protein, or a nucleic acid encoding the amino acid sequence.

Embodiment 636 is the system of Embodiment 635, wherein the DNA repair protein is a single-stranded endonuclease that removes a 5′ flap at the 3′ end of a nick.

Embodiment 637 is the system of Embodiment 636, wherein the DNA repair protein is flap structure-specific endonuclease 1 (FEN1).

Embodiment 638 is the system of Embodiment 637, wherein the FEN1 does not comprise a heterologous nuclear localization signal (NLS).

Embodiment 639 is the system of Embodiment 637 or 638, wherein the FEN1 is recruited to the nick site of the tgRNA.

Embodiment 640 is the system of any one of Embodiments 635-639, wherein the DNA repair protein comprises a heterodimerization domain or further comprises an additional heterodimerization domain.

Embodiment 641 is the system of Embodiment 640, wherein the heterodimerization] domain comprises an EV, EI, KV, or KI domain.

Embodiment 642 is the system of Embodiment 640 or 641, wherein the heterodimerization domain comprises an EI or KI domain.

Embodiment 643 is the system of any one of Embodiments 640-642, wherein the heterodimerization domain comprises SEQ ID NO: 1589, SEQ ID NO: 1593, or SEQ ID NO: 1595.

Embodiment 644 is the system of any one of Embodiments 635-636 and 640-643, wherein the DNA repair protein is MLH1.

Embodiment 645 is the system of any one of Embodiments 635-636 and 640-643, wherein the DNA repair protein is selected from:
a) a FEN1 protein comprising an amino acid sequence that has at least 90%, 91%, 92%, 93%, 94%, 95%, 96%, 97%, 98%, 99%, or 100% identity to any one of SEQ ID NOs: 1467, 1475, 1480, 1490, 1495, 1510-1516 and 1550-1555, optionally wherein the FEN1 protein includes or lacks an N-terminal methionine relative to any one of SEQ ID NOs: 1467, 1475, 1485, 1490, 1495, and 1510-1516, or a nucleic acid encoding the amino acid sequence; or
b) a MLH1 protein comprising an amino acid sequence that has at least 90%, 91%, 92%, 93%, 94%, 95%, 96%, 97%, 98%, 99%, or 100% identity to any one of SEQ ID NOs: 1572 or 1573, optionally wherein the MLH1 protein includes or lacks an N-terminal methionine relative to any one of SEQ ID NOs: 1572 or 1573, or a nucleic acid encoding the amino acid sequence.

Embodiment 646 is the system of Embodiment 629, wherein the at least two enhancers of template-based genome editing comprise an inhibitor of deoxynucleotide triphosphohydrolase and a DNA repair protein, or a nucleic acid encoding the DNA repair protein.

Embodiment 647 is the system of any one of Embodiments 629-646, wherein the at least two enhancers of template-based genome editing comprise Vpx and FEN-1.

Embodiment 648 is the system of Embodiment 647, wherein the at least two enhancers comprising Vpx and FEN1 are encoded on a same polypeptide.

Embodiment 649 is the system of Embodiment 648, wherein the polypeptide encoding Vpx and FEN1 comprises an amino acid sequence that has at least 90%, 91%, 92%, 93%, 94%, 95%, 96%, 97%, 98%, 99%, or 100% identity to any one of SEQ ID NOs: 1578, 1580, 1582, and 1584; or wherein the polypeptide encoding Vpx and FEN1 is encoded by a nucleic acid sequence that has at least 90%, 91%, 92%, 93%, 94%, 95%, 96%, 97%, 98%, 99%, or 100% identity to any one of SEQ ID NOs: 1577, 1579, 1581, and 1583, 3523, 3524, 3526, and 3527.

Embodiment 650 is the system of any one of Embodiment 629-64, wherein the at least two enhancers of template-based genome editing comprise M97 and FEN-1.

Embodiment 651 is the system of any one of Embodiments 629-650, further comprising an Sso7 DNA binding accessory factor.

Embodiment 652 is A system comprising:
a) a polK polymerase and a SpyCas9 nickase (nCas9), wherein the polK and nCas9 are covalently linked sequentially in a polypeptide from N-terminus to C-terminus wherein the order is SV40 NLS-nCas9-linker-SV40 NLS-linker-polK(1-526, GE411-412RV)-linker-SV40NLS; and
b) at least two enhancers of template-based genome editing, comprising Vpx and FEN 1.

Embodiment 653 is A system comprising:
a) a polK polymerase and a SpyCas9 nickase (nCas9), wherein the polk and nCas9 are covalently linked sequentially in a polypeptide from N-terminus to C-terminus wherein the order is SV40 NLS-nCas9-linker-SV40 NLS-linker-polK(1-526, GE411-412RV)-linker-SV40NLS; and
b) a template guide RNA (tgRNA) comprising a first polynucleotide and a second polynucleotide, wherein:
   1) the first polynucleotide comprises a SpyCas9 guide RNA comprising, from 5′ to 3′, a guide sequence and any one of SEQ ID NOs: 5, 27, 35, 210, 269, 310, 323-325, 326, 347-349, 370, 201-208, 371, 373-374, and 384-400, 438; and
   2) the second polynucleotide is operably linked to the first polynucleotide and comprises, from 5′ to 3′, a template sequence and a DNA-dependent DNA polymerase recruiting sequence (DRS).

Embodiment 654 is a system comprising:
c) a polK polymerase and a SpyCas9 nickase (nCas9), wherein the polk and nCas9 are covalently linked sequentially in a polypeptide from N-terminus to C-terminus wherein the order is SV40 NLS-nCas9-linker-SV40 NLS-linker-polK(1-526, GE411-412RV)-linker-SV40NLS;
d) a template guide RNA (tgRNA) comprising a first polynucleotide and a second polynucleotide, wherein:
   1) the first polynucleotide comprises a SpyCas9 guide RNA comprising, from 5′ to 3′, a guide sequence and any one of SEQ ID NOs: 5, 27, 35, 210, 269, 310, 322-325, 326, 347-349, 370, 201-208, 371, 373-374, and 384-400, 434, 438; and
   2) the second polynucleotide is operably linked to the first polynucleotide and comprises, from 5′ to 3′, a template sequence and a DNA-dependent DNA polymerase recruiting sequence (DRS)
e) at least at least two enhancers of template-based genome editing, comprising Vpx and FEN-1.

Embodiment 655 is the system of any one of Embodiments 652-654, wherein the polypeptide comprises the sequence of SEQ ID NO: 1234 or a nucleic acid encoding the polypeptide is provided, the nucleic acid comprising the nucleotide sequence of SEQ ID NO: 1232 or 1233.

Embodiment 656 is a system comprising:
a) a first polypeptide comprising a polK polymerase and a second polypeptide comprising a SpyCas9 nickase (nCas9), wherein:
   1) the first polypeptide comprises, from N-terminus to C-terminus, nucleoplasmin NLS-nCas9-linker-CfaN; and
   2) the second polypeptide comprises, from N-terminus to C-terminus, CfaC-linker-SV40 NLS-linker-polK-linker-SV40 NLS-c-Myc NLS; and
b) at least one enhancer of template-based genome editing, selected from Vpx and FEN 1.

Embodiment 657 is a system comprising:
c) a first polypeptide comprising a polK polymerase and a second polypeptide comprising a SpyCas9 nickase (nCas9), wherein
   3) the first polypeptide comprises, from N-terminus to C-terminus, nucleoplasmin NLS-nCas9-linker-CfaN; and
   4) the second polypeptide comprises, from N-terminus to C-terminus, CfaC-linker-SV40 NLS-linker-polK-linker-SV40 NLS-c-Myc NLS; and
d) a template guide RNA (tgRNA) comprising a first polynucleotide and a second polynucleotide, wherein:
e) the first polynucleotide comprises a SpyCas9 guide RNA comprising, from 5′ to 3′, a guide sequence and any one of SEQ ID NOs: 5, 27, 35, 210, 269, 310, 323-325, 326, 347-349, 370, 201-208, 371, 373-374, and 384-400, 438; and
f) the second polynucleotide is operably linked to the first polynucleotide and comprises, from 5′ to 3′, a template sequence and a DNA-dependent DNA polymerase recruiting sequence (DRS).

Embodiment 658 is a system comprising:
g) a first polypeptide comprising a polK polymerase and a second polypeptide comprising a SpyCas9 nickase (nCas9), wherein:
   1) the first polypeptide comprises, from N-terminus to C-terminus, nucleoplasmin NLS-nCas9-linker-CfaN; and
   2) the second polypeptide comprises, from N-terminus to C-terminus, CfaC-linker-SV40 NLS-linker-polK-linker-SV40 NLS-c-Myc NLS; and
h) a template guide RNA (tgRNA) comprising a first polynucleotide and a second polynucleotide, wherein:
   1) the first polynucleotide comprises a SpyCas9 guide RNA comprising, from 5′ to 3′, a guide sequence and any one of SEQ ID NOs: 5, 27, 35, 210, 269, 310, 323-325, 326, 347-349, 370, 201-208, 371, 373-374, and 384-400, 438; and
   2) the second polynucleotide is operably linked to the first polynucleotide and comprises, from 5′ to 3′, a template sequence and a DNA-dependent DNA polymerase recruiting sequence (DRS)
i) at least one enhancer of template-based genome editing, selected from Vpx and FEN-1.

Embodiment 659 is the system of any one of Embodiments 652-658, wherein the polk is polK(1-526, GE411-412RV).

Embodiment 660 is the system of any one of Embodiments 652-659, further comprising a nicking guide RNA (ngRNA).

Embodiment 661 is a system comprising:
a) a T5Pol polymerase and a SpyCas9 nickase (nCas9), wherein the T5Pol and nCas9 are covalently linked sequentially in a polypeptide from N-terminus to C-terminus wherein the order is nucleoplasmin NLS-nCas9-linker-SV40 NLS-linker-T5Pol-linker-SV40 NLS-c-Myc NLS; and
b) at least one enhancer of template-based genome editing, selected from Vpx and FEN-1.

Embodiment 662 is a system comprising:
a) a T5Pol polymerase and a SpyCas9 nickase (nCas9), wherein the T5Pol and nCas9 are covalently linked sequentially in a polypeptide from N-terminus to C-terminus wherein the order is nucleoplasmin NLS-nCas9-linker-SV40 NLS-linker-T5Pol-linker-SV40 NLS-c-Myc NLS; and
b) a template guide RNA (tgRNA) comprising a first polynucleotide and a second polynucleotide, wherein:
   1) the first polynucleotide comprises a SpyCas9 guide RNA comprising, from 5′ to 3′, a guide sequence and the sequence of SEQ ID NO: 5, 27, 35, 210, 269, 310, 323-325, 326, 347-349, 370, 201-208, 371, 373-374, and 384-400, and 438; and
   2) the second polynucleotide is operably linked to the first polynucleotide and comprises, from 5′ to 3′, a template sequence and a DNA-dependent DNA polymerase recruiting sequence (DRS).

Embodiment 663 is a system comprising:
c) a T5Pol polymerase and a SpyCas9 nickase (nCas9), wherein the T5Pol and nCas9 are covalently linked sequentially in a polypeptide from N-terminus to C-terminus wherein the order is nucleoplasmin NLS-nCas9-linker-SV40 NLS-linker-T5Pol-linker-SV40 NLS-c-Myc NLS;
d) a template guide RNA (tgRNA) comprising a first polynucleotide and a second polynucleotide, wherein:
   1) the first polynucleotide comprises a SpyCas9 guide RNA comprising, from 5′ to 3′, a guide sequence and the sequence of SEQ ID NO: 5, 27, 35, 210, 269, 310, 323-325, 326, 347-349, 370, 201-208, 371, 373-374, and 384-400, 438; and
   2) the second polynucleotide is operably linked to the first polynucleotide and comprises, from 5′ to 3′, a template sequence and a DNA-dependent DNA polymerase recruiting sequence (DRS)
e) at least one enhancer of template-based genome editing, selected from Vpx and FEN-1.

Embodiment 664 is the system of any one of Embodiments 661-663, wherein the polypeptide comprises the sequence of SEQ ID NO: 1219 or a nucleic acid encoding the polypeptide is provided, the nucleic acid comprising the nucleotide sequence of SEQ ID NO: 1217 or 1218.

Embodiment 665 is the system of any one of Embodiments 1-113 and 248-581, 628-664 or the fusion protein or nucleic acid of any one of Embodiments 582-627, wherein the heterologous NLS is independently selected from Nucleoplasmin NLS, C-Myc NLS, and SV40 NLS and the linker is independently selected from SEQ ID NOs: 1596, 1597, 1600, 1601, 1605, 1661, 1735, 1738, and 1815.

Embodiment 666 is the nucleic acid of any one of Embodiments 582-627 or 665, wherein the nucleotide sequence encoding a polypeptide comprising the nCas9 or DNA-dependent DNA polymerase, or the fusion protein comprises:
a) a Cap selected from Cap0 or Cap1;
b) a 5′ UTR selected from SEQ ID NOs: 1720-1723;
c) a Kozak sequence selected from SEQ ID NOs: 1724 and 1725;
d) a 3′ UTR selected from SEQ ID NOs: 1684 and 1740;
e) a poly-A tail selected from SEQ ID NOs: 1741 and 1742; or
f) a combination of two, three, four, or five of (i)-(v).

Embodiment 667 is the system of any one of Embodiments 1-113, 248-581, and 628-666, wherein the DNA-dependent DNA polymerase, nCas9, and enhancers are provided as one or more polypeptides.

Embodiment 668 is the system of any one of Embodiments 1-113, 248-581, and 628-666, wherein the DNA-dependent DNA polymerase, nCas9, and enhancers are provided as one or more nucleic acids.

Embodiment 669 is the system of any one of Embodiments 1, 248, 283, 335, 456, 628, 629, 652-654, 656-658, and 661-663, further comprising a nicking guide RNA (ngRNA).

Embodiment 670 is a method for template-based genomic editing, comprising delivering to a cell or subject the system of any one of Embodiments 1-113 and 248-581, 628-669, or the fusion protein or nucleic acid of any one of Embodiments 582-627.

Embodiment 671 is the use of the system of any one of Embodiments 1-113 and 248-581, 628-669, or the fusion protein or nucleic acid of any one of Embodiments 582-627 for template-based genome editing in a cell or subject.

Embodiment 672 is the method or use of Embodiment 670 or 671, wherein template-based genome editing results in edits comprising an insertion or deletion in a nucleotide sequence.

Embodiment 673 is the method or use of Embodiment 670 or 671, wherein template-based genome editing results in edits comprising a substitution in a nucleotide sequence.

Embodiment 674 is the method of Embodiment 673, wherein the substitution is a transition or transversion.

Embodiment 675 is the method or use of any one of Embodiments 670-673, wherein template-based genome editing results in at least 20%, 25%, 30%, 35%, 40%, 45%, optionally at least 50%, 55%, 60%, 65%, optionally at least 70%, 75%, or 80% templated edits as a percent of total edits in the target nucleic acid.

Embodiment 676 is the method or use of Embodiment 675, wherein no more than 30% of the templated edits, optionally no more than 20% or 10% of the templated edits further comprise a byproduct edit.

Embodiment 677 is the method or use of any one of Embodiments 670-673, wherein the cell is a quiescent cell and wherein at least 5% of the cells in the cell population comprise a template-based genome edit in the target nucleic acid.

Embodiment 678 is the method or use of Embodiment 677, wherein the quiescent cell is selected from a hepatocyte, unactivated T-cell, muscle cell, neuronal cell, and a lung cell.

Embodiment 679 is the method or use of Embodiment 678, wherein the hepatocyte, muscle cell, neuronal cell, or lung cell is in vivo.

Embodiment 680 is the method or use of any one of Embodiments 670-679, wherein the cell is a primary cell.

Embodiment 681 is the method or use of any one of Embodiments 670-676, wherein the cell is an immune cell and wherein at least 50% of the cells in the cell population, optionally at least 70% of the cells in the cell population, comprise a template-based genome edit in the target nucleic acid.

Embodiment 682 is the method or use of Embodiment 681, wherein no more than 30% of the templated edits, optionally no more than 20% or 10% of the templated edits further comprise a byproduct edit.

Embodiment 683 is the method or use of any one of Embodiments 670-682, wherein the length of the templated insertion is at least 5, 10, 15, 20, 25, 30, 35, or 40 nucleotides in length.

Embodiment 684 is the method or use of any one of Embodiments 670-683, wherein the length of the templated insertion is up to 500, 1000, 3500, 5000, or 10,000 nucleotides in length.

Embodiment 685 is the method or use of any one of Embodiments 670-684, wherein the length of the templated deletion is 1-3500 nucleotides in length, optionally 1-10,000 nucleotides in length.

Embodiment 686 is the method of any one of Embodiments 670-684, wherein the length of the templated insertion is 1-10, 1-20, 1-50, 1-75, or 1-100 nucleotides in length.

Embodiment 687 is the method or use of any one of Embodiments 670-686, wherein the system further comprises a nicking guide RNA (ngRNA) or the method further comprises delivering the ngRNA to a cell or subject.

Embodiment 688 is the method or use of Embodiment 687, wherein the ngRNA comprises a spacer that is complementary to the non-target strand of the DNA duplex target nucleic acid.

Embodiment 689 is the method or use of Embodiment 687 or 688, wherein the ngRNA comprises a spacer that hybridizes to a sequence in the non-target strand such that the nick site of the ngRNA is within 200 nucleotides 5′ or 3′ from the nick site of the template guide RNA.

Embodiment 690 is the method or use of Embodiment 687 or 688, wherein the ngRNA comprises a spacer that hybridizes to a sequence on the non-target strand such that the nick site of the ngRNA is 20-200 nucleotides 5′ or 3′ from the nick site of the template guide RNA.

Embodiment 691 is the method or use of Embodiment 687 or 688, wherein the ngRNA comprises a spacer that hybridizes to a sequence on the non-target strand such that the nick site of the ngRNA is within the genomic locus complementary to the spacer sequence of the template guide RNA.

Embodiment 692 is the method or use of Embodiment 687 or 688, wherein the ngRNA comprises a spacer that hybridizes to a sequence on the non-target strand such that the nick site of the ngRNA is outside of the genomic locus complementary to the spacer sequence of the template guide RNA.

Embodiment 693 is the method or use of any one of Embodiments 670-692, wherein the system further comprises at least one enhancer of template-based editing.

Embodiment 694 is the method or use of any one of Embodiments 670-693, wherein the system further comprises at least two enhancers of template-based genome editing.

Embodiment 695 is a system comprising the nucleic acid of any one of Embodiments 582-627 or a composition comprising the nucleic acid of any one of Embodiments 582-627 and an enhancer of template-based genome editing, optionally further comprising a template guide RNA of any one of Embodiments 114-247.

Embodiment 696 is the system or composition of Embodiment 695, wherein the enhancer of template-based genome editing comprises a nucleic acid encoding the enhancer of template-based genome editing or an enhancer of template-based genome editing polypeptide.

Embodiment 697 is the system or composition of Embodiment 695 or 696, wherein the enhancer of template-based genome editing is an inhibitor of deoxynucleotide triphosphohydrolase.

Embodiment 698 is the system of any one of Embodiments 695-697, wherein the deoxynucleotide triphosphohydrolase comprises SAM domain and HD domain-containing protein 1 (SAMHD1).

Embodiment 699 is the system or composition of Embodiment 698, wherein the inhibitor of SAMHD1 comprises microRNA, shRNA, or siRNA that hybridizes to a SAMHD1 mRNA.

Embodiment 700 is the system or composition of Embodiment 698, wherein the inhibitor of SAMHD1 comprises a Vpx, a BGLF4, an M97, or KSHV ORF36.

Embodiment 701 is the system or composition of Embodiment 697, wherein the enhancer of template-based genome editing is selected from:
a) a Vpx protein comprising an amino acid sequence that has at least 90%, 91%, 92%, 93%, 94%, 95%, 96%, 97%, 98%, 99%, or 100% identity to any one of SEQ ID NOs: 1464, 1469, 1503-1505, optionally wherein the Vpx protein includes or lacks an N-terminal methionine relative to any one of SEQ ID NOs: 1464, 1469, 1503-1505, or a nucleic acid encoding the amino acid sequence;
b) a BGLF4 protein comprising an amino acid sequence that has at least 90%, 91%, 92%, 93%, 94%, 95%, 96%, 97%, 98%, 99%, or 100% identity to SEQ ID NO: 1517 or 1518, optionally wherein the BGLF4 protein includes or lacks an N-terminal methionine relative to SEQ ID NO: 1517 or 1518, or a nucleic acid encoding the amino acid sequence;
c) an M97 protein comprising an amino acid sequence that has at least 90%, 91%, 92%, 93%, 94%, 95%, 96%, 97%, 98%, 99%, or 100% identity to SEQ ID NO: 1500, optionally wherein the M97 protein includes or lacks an N-terminal methionine relative to SEQ ID NO: 1500, or a nucleic acid encoding the amino acid sequence; or
d) a KSHV ORF36 protein comprising an amino acid sequence that has at least 90%, 91%, 92%, 93%, 94%, 95%, 96%, 97%, 98%, 99%, or 100% identity to SEQ ID NO: 1576, optionally wherein the KSHV ORF36 protein includes or lacks an N-terminal methionine relative to SEQ ID NO: 1576, or a nucleic acid encoding the amino acid sequence.

Embodiment 702 is the system or composition of Embodiment 695 or 696, wherein the enhancer of template-based genome editing is a DNA repair protein, or a nucleic acid encoding the amino acid sequence.

Embodiment 703 is the system or composition of Embodiment 702, wherein the wherein the DNA repair protein is a single-stranded endonuclease that removes a 5′ flap at the 3′ end of a nick.

Embodiment 704 is the system or composition of Embodiment 703, wherein the DNA repair protein is flap structure-specific endonuclease 1 (FEN1).

Embodiment 705 is the system or composition of Embodiment 704, wherein the FEN1 does not comprise a heterologous nuclear localization signal (NLS).

Embodiment 706 is the system or composition of Embodiment 704 or 705, wherein the FEN1 is recruited to the nick site of the tgRNA.

Embodiment 707 is the system or composition of any one of Embodiments 702-706, wherein the DNA repair protein comprises a heterodimerization domain or further comprises an additional heterodimerization domain.

Embodiment 708 is the system or composition of Embodiment 707, wherein the heterodimerization domain comprises a coiled coil heterodimerization domain.

Embodiment 709 is the system or composition of Embodiment 707 or 708, wherein the heterodimerization domain comprises an EV, EI, KV, or KI domain.

Embodiment 710 is the system or composition of any one of Embodiments 707-709, wherein the heterodimerization domain comprises an EI or KI domain.

Embodiment 711 is the system or composition of any one of Embodiments 707-710, wherein the heterodimerization domain comprises SEQ ID NO: 1589, SEQ ID NO: 1593, or SEQ ID NO: 1595.

Embodiment 712 is the system or composition of any one of Embodiments 702-703 and 707-711, wherein the DNA repair protein is MLH1.

Embodiment 713 is the system or composition of any one of Embodiments 702-703 and 707-711, wherein the DNA repair protein is selected from:
a) a FEN1 protein comprising an amino acid sequence that has at least 90%, 91%, 92%, 93%, 94%, 95%, 96%, 97%, 98%, 99%, or 100% identity to any one of SEQ ID NOs: 1467, 1475, 1485, 1490, 1495, and 1510-1516, optionally wherein the FEN1 protein includes or lacks an N-terminal methionine relative to any one of SEQ ID NOs: 1467, 1475, 1485, 1490, 1495, and 1510-1516, or a nucleic acid encoding the amino acid sequence; or
b) MLH1 protein comprising an amino acid sequence that has at least 90%, 91%, 92%, 93%, 94%, 95%, 96%, 97%, 98%, 99%, or 100% identity to any one of SEQ ID NOs: 1572 or 1573, optionally wherein the MLH1 protein includes or lacks an N-terminal methionine relative to any one of SEQ ID NOs: 1572 or 1573, or a nucleic acid encoding the amino acid sequence.

Embodiment 714 is the system or composition of any one of Embodiments 695-713, wherein the enhancer of template-based genome editing does not comprise a heterologous NLS.

Embodiment 715 is the system or composition of any one of Embodiments 695-713, wherein the enhancer of template-based genome editing comprises a heterologous NLS.

Embodiment 716 is the system or composition of Embodiment 715, wherein the NLS is selected from a c-Myc NLS, an SV40 NLS, a nucleoplasmin NLS, or a snurportin-1 importin-β NLS.

Embodiment 717 is the system or composition of any one of Embodiments 695-716 comprising two nucleic acids encoding two enhancers of template-based genome editing.

Embodiment 718 is the system or composition of Embodiment 717, comprising Vpx and FEN-1.

Embodiment 719 is the system or composition of Embodiment 717, comprising M97 and FEN-1.

Embodiment 720 is the system or composition of any one of Embodiments 308-332, further comprising an Sso7 DNA binding accessory factor.

Embodiment 721 is the system or composition of Embodiment 720, wherein the Sso7 DNA binding accessory factor comprises an amino acid sequence that has at least 90%, 91%, 92%, 93%, 94%, 95%, 96%, 97%, 98%, 99%, or 100% identity to any one of SEQ ID NOs: 1547, or a nucleic acid encoding the amino acid sequence.

Embodiment 722 is the system or composition of any one of Embodiments 1-113, 248-581, and 695-721, wherein further comprises a nicking guide RNA (ngRNA).

Embodiment 723 is the system or composition of Embodiment 722, wherein the ngRNA comprises a spacer that is complementary to the non-target strand of the DNA duplex target nucleic acid.

Embodiment 724 is the system or composition of Embodiment 723, wherein the ngRNA comprises a spacer that hybridizes to a sequence on the non-target strand such that the nick site of the ngRNA is within 200 nucleotides 5′ or 3′ from the nick site of the template guide RNA.

Embodiment 725 is the system or composition of any one of Embodiments 722-724, wherein the ngRNA comprises a spacer that hybridizes to a sequence on the non-target strand such that the nick site of the ngRNA is about 20-200 nucleotides 5′ or 3′ from the nick site of the template guide RNA.

Embodiment 726 is the system or composition of any one of Embodiments 722-724, wherein the ngRNA comprises a spacer that hybridizes to a sequence on the non-target strand such that the nick site of the ngRNA is outside of the genomic locus of the spacer sequence of the template guide RNA.

Embodiment 727 is the system or composition of any one of Embodiments 1-113, 248-581, and 695-726, wherein the nCas9 comprises an amino acid sequence that has at least 90%, 91%, 92%, 93%, 94%, 95%, 96%, 97%, 98%, 99%, or 100% identity to any one of SEQ ID NOs: 972, 974, 976, 982, 984, 986, 988, 990, 992, 994, 996, 998, 1000, and 1014, optionally wherein the nCas9 protein includes or lacks an N-terminal methionine relative to any one of SEQ ID NOs: 972, 974, 976, 982, 984, 986, 988, 990, 992, 994, 996, 998, 1000, and 1014.

Embodiment 728 is the system or composition of Embodiment 727, wherein the nCas9 comprises an amino acid sequence of any one of SEQ ID NOs: 972, 974, 976, 982, 984, 986, 988, 990, 992, 994, 996, 998, 1000, and 1014.

Embodiment 729 is the system or composition of any one of Embodiments 1-113, 248-581, and 695-728, wherein the nCas9 is encoded by a nucleotide sequence that has at least 90%, 91%, 92%, 93%, 94%, 95%, 96%, 97%, 98%, 99%, or 100% identity to any one of SEQ ID NOs: 971, 973, 975, 981, 983, 985, 987, 989, 991, 993, 995, 997, 999, and 1013, optionally wherein the nCas9 protein includes or lacks a start codon or stop codon relative to any one of SEQ ID NOs: 971, 973, 975, 981, 983, 985, 987, 989, 991, 993, 995, 997, 999, and 1013.

Embodiment 730 is the system or composition of Embodiment 729, wherein the nCas9 is encoded by a nucleotide sequence of any one of SEQ ID NOs: 971, 973, 975, 981, 983, 985, 987, 989, 991, 993, 995, 997, 999, and 1013.

Embodiment 731 is the system or composition of any one of Embodiments 1-113, 248-581, and 695-730, wherein the nCas9 comprises an H840A point mutation.

Embodiment 732 is the system or composition of any one of Embodiments 695-731, wherein the nucleotide sequence encoding the nCas9, DNA-dependent DNA polymerase, nCas9/DNA-dependent DNA polymerase fusion protein, enhancer of template-based genome editing, or Sso7 DNA binding accessory factor comprises a 5′ cap selected from Cap0, Cap1, and Cap2.

Embodiment 733 is the system or composition of any one of Embodiments 695-732, wherein the polynucleotide further comprises a poly-adenylated (poly-A) tail sequence or a polyadenylation signal sequence.

Embodiment 734 is the system or composition of Embodiment 733, wherein the poly-A tail sequence is not encoded, optionally wherein the poly-A tail sequence is added enzymatically.

Embodiment 735 is the system or composition of Embodiment 733, wherein the poly-A tail sequence is an encoded poly-A tail sequence.

Embodiment 736 is the system or composition of any one of Embodiments 733-735, wherein the poly-A sequence comprises non-adenine nucleotides.

Embodiment 737 is the system or composition of any one of Embodiments 733-736, wherein the poly-A sequence comprises 100-400 nucleotides.

Embodiment 738 is the system or composition of any one of Embodiments 733-737, wherein the poly-A sequence comprises a sequence of any one of SEQ ID NOs: 1692-1693 and 1741-1742.

Embodiment 739 is the system or composition of any one of Embodiments 695-738, wherein at least 70%, 75%, 80%, 85%, 90%, or 95% of the uridine in the nucleic acid is substituted with a modified uridine.

Embodiment 740 is the system or composition of Embodiment 739, wherein the modified uridine is one or more of N1-methyl-pseudouridine, pseudouridine, 5-methoxyuridine, or 5-iodouridine.

Embodiment 741 is the system or composition of Embodiment 739, wherein the modified uridine is N1-methyl-pseudouridine or 5-methoxyuridine.

Embodiment 742 is the system or composition of Embodiment 739, wherein the modified uridine is N1-methyl-pseudouridine.

Embodiment 743 is the system or composition of Embodiment 739, wherein the modified uridine is 5-methoxyuridine.

Embodiment 744 is the system or composition of any one of Embodiments 1-113, 248-581, 628-669, and 695-743, further comprising a template guide RNA, if not present, wherein the template guide RNA is a template guide RNA of Embodiment 114.

Embodiment 745 is the system or composition of Embodiment 744, wherein the DRS bound to the DRS-complementary region in the non-target strand of the DNA duplex target nucleic acid forms a binding site for a DNA-dependent DNA polymerase, optionally wherein the DNA-dependent DNA polymerase is polK or T5 pol.

Embodiment 746 is the system or composition of Embodiment 744 or 745, wherein the DRS comprises a 3′ tail.

Embodiment 747 is the system or composition of Embodiment 746, wherein the 3′ tail comprises 1, 2, 3, 4, or 5 nucleotides, optionally 1-3 nucleotides.

Embodiment 748 is the system or composition of Embodiment 746 or 747, wherein the 3′ tail comprises a terminal uridine nucleotide or, wherein the 3′ tail consists of a uridine nucleotide.

Embodiment 749 is the system or composition of any one of Embodiments 745-748, wherein the tail comprises RNA nucleotides.

Embodiment 750 is the system or composition of any one of Embodiments 745-749, wherein the tail comprises only RNA nucleotides.

Embodiment 751 is the system or composition of any one of Embodiments 745-750, wherein the 3′ tail comprises modified RNA nucleotides.

Embodiment 752 is the system or composition of any one of Embodiments 745-751, wherein the 3′ tail consists of modified RNA nucleotides.

Embodiment 753 is the system or composition of any one of Embodiments 745-752, wherein the 3′ terminal nucleotide is a modified RNA nucleotide.

Embodiment 754 is the system or composition of any one of Embodiments 745-753, wherein the modified nucleotide comprises a modification selected from 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, or an inverted abasic modified nucleotide, optionally 2′-O-me and phosphorothioate.

Embodiment 755 is the system or composition of any one of Embodiments 744-754, wherein the DRS comprises a 3′ end and a 5′ end, wherein 0-7 nucleotides at the 5′ end of the DRS are DNA nucleotides (nucleotides D1-D7).

Embodiment 756 is the system of any one of Embodiments 744-754, wherein one or more of nucleotides 1-5 at the 5′ end of the DRS (nucleotides D1-D5) are DNA nucleotides, optionally wherein the remaining nucleotides in the DRS are RNA nucleotides.

Embodiment 757 is the system or composition of any one of Embodiments 744-754, and 756, wherein 1-3 nucleotides at the 5′ end of the DRS (nucleotides D1-D3), optionally the most 5′ nucleotide in the DRS (nucleotide D1), is a DNA nucleotide.

Embodiment 758 is the system or composition of any one of Embodiments 744-757, wherein nucleotides 1 and 3 at the 5′ end of the DRS (nucleotides D1 and D3) are DNA nucleotides, optionally wherein the remaining nucleotides in the DRS are RNA nucleotides.

Embodiment 759 is the system of any one of Embodiments 744-758, wherein nucleotides 1, 3, and 5 at the 5′ end of the DRS (nucleotides D1, D3, and D5) are DNA nucleotides, optionally wherein the remaining nucleotides in the DRS are RNA nucleotides.

Embodiment 760 is the system or composition of any one of Embodiments 744 wherein the 5′ terminal nucleotide in the DRS (nucleotide D1) is a DNA nucleotide.

Embodiment 761 is the system or composition of any one of Embodiments 744 wherein each nucleotide from the penultimate 5′ terminal nucleotide to the 3′ terminal nucleotide in the DRS (nucleotide D2) is an RNA nucleotide.

Embodiment 762 is the system of any one of Embodiments 744-761, wherein the 3′ terminal nucleotide in the DRS is an RNA nucleotide and the remaining nucleotides in the DRS are DNA nucleotides.

Embodiment 763 is the system or composition of any one of Embodiments 744-762, wherein the first polynucleotide is not covalently linked to the second polynucleotide.

Embodiment 764 is the system or composition of Embodiment 763, wherein 1-7 consecutive 5′ terminal nucleotides of the DRS are DNA nucleotides.

Embodiment 765 is the system or composition of Embodiment 763 or 764, wherein the 5′ terminal and penultimate 5′ terminal nucleotides of the DRS are DNA nucleotides.

Embodiment 766 is the system or composition of any one of Embodiments 763-765, wherein at least 3 consecutive 5′ terminal nucleotides of the DRS are DNA nucleotides.

Embodiment 767 is the system or composition of any one of Embodiments 763-766, wherein 3 consecutive 5′ terminal nucleotides of the DRS are DNA nucleotides.

Embodiment 768 is the system or composition of any one of 744-754, wherein the DRS consists of RNA nucleotides.

Embodiment 769 is the system or composition of any one of Embodiments 1744-768, wherein the DRS comprises a modified RNA nucleotide.

Embodiment 770 is the system or composition of Embodiment 769, wherein the modified RNA nucleotide is selected from:
a) 2′-O-methyl (2′-O-Me) modified nucleotide;
b) 2′-fluoro (2′-F) modified nucleotide;
c) an amine modified nucleotide;
d) an alkoxy modified nucleotide;
e) an LNA modified nucleotide;
f) methoxyethyl modified nucleotide;
g) a halo modified nucleotide; or
h) phosphorothioate (PS) linkage modified nucleotide;
optionally, wherein the modified nucleotide is a 2′-O-methyl (2′-O-Me) modified nucleotide or a phosphorothioate (PS) linkage modified nucleotide.

Embodiment 771 is the system or composition of Embodiment 769 or 770, wherein 1-3 terminal nucleotides at the 3′ end of the DRS are chemically modified, optionally wherein the nucleotide at the 3′ end of the DRS is chemically modified.

Embodiment 772 is the system or composition of any one of Embodiments 769-771, wherein 1-3 terminal nucleotides at the 3′ end of the DRS comprises a phosphorothioate (PS) modification, optionally wherein at least one PS modification is between the last two nucleotides at the 3′ terminus.

Embodiment 773 is the system or composition of any one of Embodiments 769-772, wherein the 3′ terminal nucleotide of the DRS comprises a 2′-O-Me modification.

Embodiment 774 is the system or composition of any one of Embodiments 769-773, wherein the DRS comprises PS linkages between 2, 3, 4, 5, or 6 consecutive 3′ terminal nucleotides of the DRS

Embodiment 775 is the system or composition of any one of Embodiments 769-774, wherein the DRS comprises a PS linkage between the 3′ terminal and penultimate 3′ terminal nucleotides of the DRS.

Embodiment 776 is the system or composition of any one of Embodiments 769-775, wherein the DRS comprises three PS linkages between the 4 consecutive 3′ terminal nucleotides of the DRS.

Embodiment 777 is the system or composition of any one of Embodiments 769-776, wherein the DRS comprises a PS linkage between two nucleotides other than the 3′ terminal and penultimate 3′ terminal nucleotides of the DRS.

Embodiment 778 is the system or composition of any one of Embodiments 769-777, wherein the DRS comprises a 2′-O-Me modification at a nucleotide other than the 5′ terminal and 3′ terminal nucleotides of the DRS.

Embodiment 779 is the system or composition of any one of Embodiments 769-778, wherein the DRS comprises a phosphorothioate modification at or 3′ of position D10 in the DRS sequence.

Embodiment 780 is the system or composition of any one of Embodiments 769-779, wherein the DRS comprises a phosphorothioate modification, and wherein the DRS does not comprise a phosphorothioate modification at position D1-D9 in the DRS sequence.

Embodiment 781 is the system or composition of any one of Embodiments 769-780, wherein the DRS comprises a 2′-O-Me modification at or 3′ of position D4 in the DRS sequence.

Embodiment 782 is the system or composition of any one of Embodiments 769-781, wherein the DRS comprises a 2′-O-Me modification, and wherein the DRS does not comprise a 2′-O-Me modification at position D1-D3 in the DRS sequence.

Embodiment 783 is the system or composition of any one of Embodiments 744-782, wherein all RNA nucleotides in the DRS comprise modified RNA nucleotides.

Embodiment 784 is the system or composition of any one of Embodiments 744, wherein the DRS is 6-20 nucleotides in length.

Embodiment 785 is the system or composition of any one of Embodiments 744-784, wherein the DRS is 6-17, 8-17, 10-12, 10-14, 10-16, or 12-14 nucleotides in length.

Embodiment 786 is the system or composition of any one of Embodiments 744-785, wherein the DRS is 10-16 nucleotides in length.

Embodiment 787 is the system or composition of any one of Embodiments 744-786, wherein the DRS has 0, 1, 2, 3, or 4 mismatches to the corresponding genomic sequence.

Embodiment 788 is the system or composition of any one of Embodiments 744-787, wherein 6 terminal nucleotides at the 5′ end of the DRS have 0, 1, 2, or 3 mismatches to the corresponding genomic sequence.

Embodiment 789 is the system or composition of any one of Embodiments 744-788, wherein the first 3 nucleotides at the 3′ end of the DRS have 0, 1, or 2 mismatches to the genomic sequence.

Embodiment 790 is the system or composition of any one of Embodiments 744-789, wherein the DRS is 100% complementary to the corresponding genomic sequence for no more than 6 consecutive nucleotides, optionally no more than 4 consecutive nucleotides, without a mismatch interruption.

Embodiment 791 is the system or composition of any one of Embodiments 744-790, wherein 10 terminal nucleotides at the 5′ end of the DRS are 100% complementary to the corresponding genomic sequence.

Embodiment 792 is the system or composition of any one of Embodiments 787-791, wherein the at least one mismatched RNA nucleotides in the DRS comprises a modified RNA nucleotide.

Embodiment 793 is the system or composition of any one of Embodiments 787-792, wherein all of the mismatched RNA nucleotides in the DRS comprise a modified RNA nucleotide.

Embodiment 794 is the system or composition of Embodiment 792 or 793, wherein the modified RNA nucleotide at the mismatched RNA nucleotide in the DRS is selected from:
a) 2′-O-methyl (2′-O-Me) modified nucleotide;
b) 2′-fluoro (2′-F) modified nucleotide;
c) an amine modified nucleotide;
d) an alkoxy modified nucleotide;
e) an LNA modified nucleotide;
f) methoxyethyl modified nucleotide;
g) a halo modified nucleotide;
h) phosphorothioate (PS) linkage modified nucleotide; or
i) optionally 2′O-me modified RNA nucleotide.

Embodiment 795 is the system or composition of any one of Embodiments 744-794, wherein the template sequence is 10-10000 nucleotides in length.

Embodiment 796 is the system or composition of any one of Embodiments 744-795, wherein the template sequence is 10-500, optionally 10-100 nucleotides in length.

Embodiment 797 is the system or composition of any one of Embodiments 744-796, wherein the template sequence is 10-20 nucleotides in length.

Embodiment 798 is the system or composition of any one of Embodiments 744-797, wherein the template sequence is 14-18 nucleotides in length, optionally 14 or 15 nucleotides in length.

Embodiment 799 is the system or composition of any one of Embodiments 744-798, wherein the template sequence further comprises at least 2 nucleotides 5′ of a sequence to be edited by template-based genome editing, wherein the at least 8 nucleotides across the template base pair with the non-target strand of the DNA duplex target nucleic acid.

Embodiment 800 is the system or composition of any one of Embodiments 744-799, wherein the template sequence further comprises at least 5 nucleotides 5′ of a sequence to be edited by template-based genome editing, wherein the at least 10 nucleotides across the template base pair with the non-target strand of the DNA duplex target nucleic acid.

Embodiment 801 is the system or composition of any one of Embodiments 744-800, wherein the template sequence comprises a sequence of at least 5 consecutive nucleotides that base pair with the non-target strand of the DNA duplex target nucleic acid.

Embodiment 802 is the system or composition of any one of Embodiments 744-801, wherein the template sequence comprises a mismatch to the genomic sequence at at least one of positions 1-6, optionally at at least one of positions 5-6 5′ of the template junction with the DRS (positions T1-T6, optionally positions T5-T6).

Embodiment 803 is the system or composition of Embodiment 802, wherein the template sequence comprises a mismatch to the genomic sequence at at least one of positions 1-6, optionally at at least one of positions 5-6 5′ of the template junction with the DRS introduces a silent mutation.

Embodiment 804 is the system or composition of Embodiment 802, wherein the template sequence comprises a mismatch to the genomic sequence at at least one of positions T1-T6, optionally at at least one of positions T5-T6 5′ of the template junction with the DRS, wherein the template introduces a change in the amino acid sequence encoded by the genomic sequence.

Embodiment 805 is the system or composition of any one of Embodiments 744-804, wherein the template sequence comprises an insertion sequence relative to the corresponding genomic sequence.

Embodiment 806 is the system or composition of Embodiment 805, wherein the template sequence comprises an insertion sequence of up to 500, 1000, 3500, or 5000 nucleotides in length, optionally up to 10,000 nucleotides in length.

Embodiment 807 is the system or composition of Embodiment 805 or 806, wherein the length of the templated insertion is 1-10, 1-20, 1-50, 1-75, or 1-100 nucleotides in length.

Embodiment 808 is the system or composition of any one of Embodiments 805-807, wherein the template comprises a nucleotide sequence 5′ of the insertion sequence that is at least 5 nucleotides, optionally at least 10, 20, 30, 40, or 50 nucleotides complementary to the genomic sequence of the non-target strand of the DNA duplex target nucleic acid.

Embodiment 809 is the system or composition of any one of Embodiments 805-808, wherein the template comprises a nucleotide sequence 5′ of the insertion sequence that is at least 5 nucleotides, optionally at least 10 nucleotides complementary to the genomic sequence of the non-target strand of the DNA duplex target nucleic acid.

Embodiment 810 is the system or composition of any one of Embodiments 744-804, wherein the template sequence comprises a sequence for directing a deletion relative to the corresponding genomic sequence.

Embodiment 811 is the system or composition of Embodiment 810, wherein the template sequence comprises a sequence to direct a deletion of at least 3, 10, 15, 20, 30, 40, or 50 nucleotides in length.

Embodiment 812 is the system or composition of Embodiment 810 or 811, wherein the template sequence comprises a sequence to direct a deletion of up to 3500 nucleotides in length, optionally up to 10,000 nucleotides in length.

Embodiment 813 is the system or composition of any one of Embodiments 810-812, wherein the template comprises a nucleotide sequence 5′ of the sequence to direct a deletion that is at least 10 nucleotides, optionally at least 20, 30, 40, or 50 nucleotides complementary to the genomic sequence of the non-target strand of the DNA duplex target nucleic acid.

Embodiment 814 is the system or composition of any one of Embodiments 805-813, wherein the insertion sequence or sequence for directing a deletion in the template sequence disrupts the spacing between a spacer target sequence and a PAM.

Embodiment 815 is the system or composition of any one of Embodiments 744-814, wherein the template sequence comprises at least one mismatch, optionally at least 3 mismatches relative to the corresponding genomic sequence wherein the mismatch provides the template sequence for template-based genome editing comprising a substitution.

Embodiment 816 is the system or composition of any one of Embodiments 744-815, wherein the template sequence comprises 1-10 mismatches relative to the corresponding genomic sequence wherein the mismatch provides the template sequence for template-based genome editing comprising a substitution.

Embodiment 817 is the system or composition of Embodiment 815 or 816, wherein the substitution is a transition or transversion.

Embodiment 818 is the system or composition of any one of Embodiment 815-817, wherein the template-based edit in the genomic sequence results in a change in an amino acid sequence encoded by a locus in which the genomic sequence is present.

Embodiment 819 is the system or composition of any one of Embodiments 815-817, wherein the template-based edit in the genomic sequence does not result in a change in an amino acid sequence encoded by a locus in which the genomic sequence is present.

Embodiment 820 is the system or composition of any one of Embodiments 815-819, wherein the template further comprises an insertion sequence or a sequence for directing a deletion.

Embodiment 821 is the system or composition of any one of Embodiments 744-820, wherein the template comprises both DNA nucleotides and RNA nucleotides.

Embodiment 822 is the system or composition of any one of Embodiments 744-821, wherein the template comprises 1-3 RNA nucleotides, optionally 2 RNA nucleotides, optionally only 1 RNA nucleotide, at the 3′ end of the template (T1-T3, optionally T1 and T2, optionally T1).

Embodiment 823 is the system or composition of Embodiment 822, wherein at least one nucleotide at positions T1 or T2 of the template is an RNA nucleotide.

Embodiment 824 is the system or composition of any one of Embodiments 744-823, wherein the template consists of DNA nucleotides from nucleotide 4 from the 3′ end of the template (T4) to the 5′ end of the template.

Embodiment 825 is the system or composition of any one of Embodiments 744-821 or 824, wherein the template consists of DNA nucleotides.

Embodiment 826 is the system or composition of any one of Embodiments 744-825, wherein the tgRNA further comprises an affinity tag, wherein the first polynucleotide or the second polynucleotide are bound to a peptide or a peptide complex comprising the nCas9 and the DNA-dependent DNA polymerase by the affinity tag.

Embodiment 827 is the system or composition of Embodiment 826, wherein 1-7 consecutive 5′ terminal nucleotides of the DRS are DNA nucleotides.

Embodiment 828 is the system or composition of Embodiment 826 or 827, wherein the 5′ terminal and penultimate 5′ terminal nucleotides of the DRS are DNA nucleotides.

Embodiment 829 is the system or composition of any one of Embodiments 826-828, wherein at least 3 consecutive 5′ terminal nucleotides of the DRS are DNA nucleotides.

Embodiment 830 is the system or composition of any one of Embodiments 826-829, wherein 3 consecutive 5′ terminal nucleotides of the DRS are DNA nucleotides.

Embodiment 831 is the system, tgRNA, or composition of any one of Embodiments 1-581, 628-669, and 695-830, wherein the SpyCas9 guide RNA scaffold sequence comprises an internal linker.

Embodiment 832 is the system, tgRNA, or composition of any one of Embodiments 1-581, 628-669, and 695-831, wherein the SpyCas9 guide RNA scaffold sequence comprises (i) an internal linker in or substitutes for the upper stem region; (ii) an internal linker in a nexus region; or (iii) an internal linker in the hairpin 1 region.

Embodiment 833 is the system, tgRNA, or composition of any one of Embodiments 831 or 832, wherein the internal linker in the upper stem region substitutes for US1 through US12 relative to SEQ ID NO: 22.

Embodiment 834 is the system, tgRNA, or composition of any one of Embodiments 831-833, wherein the internal linker in the hairpin 1 substitutes for H1-5 through H1-8 relative to SEQ ID NO: 22.

Embodiment 835 is the system, tgRNA, or composition of any one of Embodiments 831-834, wherein the internal linker has a bridging length of about 3 Angstroms-37 Angstroms.

Embodiment 836 is the system, tgRNA, or composition of any one of Embodiments 831-835, wherein the internal linker has a bridging length of about 7-22 Angstroms.

Embodiment 837 is the system, tgRNA, or composition of any one of Embodiments 831-836, wherein the internal linker comprises a structure of formula (I):

~-L0-L1-L2-# (I)
wherein:
~ indicates a bond to a 3′ substituent of the preceding nucleotide;
# indicates a bond to a 5′ substituent of the following nucleotide;
L0 is null or C1-3 aliphatic;
L1 is -[E1-(R1)]m-, where
each R1 is independently a C1-5 aliphatic group, optionally substituted with 1 or 2 E2,
each E1 and E2 are independently a hydrogen bond acceptor, or are each independently chosen from cyclic hydrocarbons, and heterocyclic hydrocarbons, and
each m is 1, 2, 3, 4, 5, 6, 7, 8, 9, or 10; and
L2 is null, C1-3 aliphatic, or is a hydrogen bond acceptor.

Embodiment 838 is the system, tgRNA, or composition of any one of Embodiments 831-837, wherein one of the internal linkers is in the repeat/anti-repeat region.

Embodiment 839 is the system, tgRNA, or composition of any one of Embodiments 831-838, further comprising an internal linker in a nexus region.

Embodiment 840 is the system, tgRNA, or composition of any one of Embodiments 831-839, wherein one of the internal linkers is in a hairpin 1 region.

Embodiment 841 is the system, tgRNA, or composition of any one of Embodiments 831-840, wherein nucleotides 29-40 (US1 through US12 of the upper stem region) are substituted for the first internal linker relative to SpyCas9 guide RNA reference sequence (SEQ ID NO: 22); and nucleotides 70-78 (HP1-HP10 of the hairpin 1 region) are substituted for the second internal linker relative to SpyCas9 guide RNA reference sequence (SEQ ID NO: 22).

Embodiment 842 is the system, tgRNA, or composition of any one of Embodiments 831-841, wherein the guide RNA comprises
a) the sequence of GUUUUAGA(L3)AAGUUAAAAUAAGGCUAGUCCGUUAUCAC(L1)GGGCAC CGAGUCGGUGCU (SEQ ID NO: 370) or a sequence having at least 90%, 95%, or 98% identity to SEQ ID NO: 370;
b) the sequence of GUUUUAGAGC(L1)GCAAGUUAAAAUAAGGCUAGUCCGUUAUCAC(L1) GG GCACCGAGUCGGUGC (SEQ ID NO: 429) or a sequence having at least 90%, 95%, 98%, 99% identity to SEQ ID NO: 429; or
c) the sequence of GUUUUAGAGCUA(L1)UAGCAAGUUAAAAUAAGGCUAGUCCGUUAUCAC(L1)GGGCACCGAGUCGGUGC (SEQ ID NO: 430) or a sequence having at least 90%, 95%, 98%, 99% identity to SEQ ID NO: 430, wherein L1 denotes that the linker is S18, and L3 denotes that the linker is S6.

Embodiment 843 is the system, tgRNA, or composition of any one of Embodiments 1-581, 628-669, and 695-842, wherein the spacer or the scaffold of the guide RNA comprises a modified nucleotide.

Embodiment 844 is the system, tgRNA, or composition of any one of Embodiments 1-581, 628-669, and 695-842, wherein the spacer of the guide RNA comprises a DNA nucleotide.

Embodiment 845 is the system, tgRNA, or composition of any one of Embodiments 1-581, 628-669, and 695-844, wherein the spacer or the scaffold of the guide RNA comprises a modified nucleotide selected from a 2′-O-methyl (2′-O-Me) modified nucleotide, or a 2′-F modified nucleotide.

Embodiment 846 is the system, tgRNA, or composition of any one of Embodiments 1-581, 628-669, and 695-845, wherein the spacer or the scaffold of the guide RNA comprises a phosphorothioate (PS) bond between nucleotides.

Embodiment 847 is the system, tgRNA, or composition of any one of Embodiments 1-581, 628-669, and 695-846, wherein the guide RNA comprises a modified nucleotide at one or more of the first five nucleotides at the 5′ end of the spacer.

Embodiment 848 is the system, tgRNA, or composition of any one of Embodiments 1-581, 628-669, and 695-847, wherein the guide RNA comprises a modified nucleotide at one or more of the last five nucleotides at the 3′ end of the scaffold.

Embodiment 849 is the system, tgRNA, or composition of any one of Embodiments 1-581, 628-669, and 695-848, wherein the guide RNA comprises a DNA nucleotide at at least 2, 3, 4, 5, 6, 7, 8, 9, or 10 nucleotides of the spacer.

Embodiment 850 is the system, tgRNA, or composition of any one of Embodiments 1-581, 628-669, and 695-849, wherein the guide RNA comprises a DNA nucleotide at 1, 2, 3, 4, 5, 6, 7, 8, 9, 10, or 11 nucleotides of the spacer.

Embodiment 851 is the system, tgRNA, or composition of any one of Embodiments 1-581, 628-669, and 695-850, wherein the guide RNA comprises a DNA nucleotide at one or more of positions S11, S12, and S18.

Embodiment 852 is the system, tgRNA, or composition of any one of Embodiments 1-581, 628-669, and 695-851, wherein the guide RNA comprises a PS bond between each of the first four nucleotides at the 5′ end of the spacer.

Embodiment 853 is the system, tgRNA, or composition of any one of Embodiments 1-581, 628-669, and 695-852, wherein the guide RNA comprises a PS bond between each of the last four nucleotides at the 3′ end of the scaffold.

Embodiment 854 is the system, tgRNA, or composition of any one of Embodiments 1-581, 628-669, and 695-853, wherein the guide RNA comprises a 2′-O-Me or 2′-F modified nucleotide at at least 2, 3, 4, 5, 6, 7, 8, 9, or 10 nucleotides of the spacer.

Embodiment 855 is the system, tgRNA, or composition of any one of Embodiments 1-581, 628-669, and 695-854, wherein the guide RNA comprises 2′-O-Me or 2′-F modified nucleotides at one or more of positions S4, S5, S9, S10, S13, S14, S17 and S20.

Embodiment 856 is the system, tgRNA, or composition of any one of Embodiments 1-581, 628-669, and 695-855, wherein the guide RNA comprises a 2′-O-Me modified nucleotide at each of the first three nucleotides at the 5′ end of the spacer.

Embodiment 857 is the system, tgRNA, or composition of any one of Embodiments 1-581, 628-669, and 695-856, wherein the guide RNA comprises a 2′-O-Me modified nucleotide at each of the last three or four nucleotides at the 3′ end of the scaffold.

Embodiment 858 is the system, tgRNA, or composition of any one of Embodiments 1-581, 628-669, and 695-857, wherein the guide RNA terminates with 1-4 uridine nucleotides or 1-4 modified uridine nucleotides, optionally a modified uridine nucleotide at the 3′ end.

Embodiment 859 is the system, tgRNA, or composition of any one of Embodiments 1-581 and 695-858, wherein the guide RNA comprises a 3′ end modification and 5′ end modification.

Embodiment 860 is the system, tgRNA, or composition of Embodiment 859, wherein the 3′ end modification or the 5′ end modification comprises a protective end modification, optionally a modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, or an inverted abasic modified nucleotide.

Embodiment 861 is the system, tgRNA, or composition of Embodiment 859 or 860, wherein the 3′ end modification or 5′ end modification comprises or further comprises a 2′-O-methyl (2′—O-Me) modified nucleotide.

Embodiment 862 is the system, tgRNA, or composition of any one of Embodiments 1-581, 628-669, and 695-861, wherein the guide RNA terminates with 1-4 uridine nucleotides or 1-4 modified uridine nucleotides.

Embodiment 863 is the system, tgRNA, or composition of any one of Embodiments 859-862, wherein the 3′ end modification or 5′ end modification comprises or further comprises a phosphorothioate (PS) linkage between nucleotides.

Embodiment 864 is the system, tgRNA, or composition of any one of Embodiments 1-581, 628-669, and 695-863, wherein the guide RNA comprises a modification in a hairpin 1 region or a hairpin 2 region.

Embodiment 865 is the system, tgRNA, or composition of any one of Embodiments 1-581, 628-669, and 695-864, comprising a modification in the hairpin 1 or hairpin 2 region, wherein the modification in the hairpin 1 or hairpin 2 region comprises a modified nucleotide selected from a 2′-O-methyl (2′-O-Me) modified nucleotide, a 2′-fluoro (2′-F) modified nucleotide, or a phosphorothioate (PS) linkage between nucleotides.

Embodiment 866 is the system, tgRNA, or composition of Embodiment 864 or 865, wherein the modification in the hairpin 1 or hairpin 2 region comprises or further comprises a 2′-O-methyl (2′-Ome) modified nucleotide.

Embodiment 867 is the system, tgRNA, or composition of any one of Embodiments 864-866, wherein the modification in the hairpin 1 or hairpin 2 region comprises or further comprises a phosphorothioate (PS) linkage between nucleotides.

Embodiment 868 is the system, tgRNA, or composition of any one of Embodiments 1-581, 628-669, and 695-867, wherein the guide RNA comprises a modification in a bulge region.

Embodiment 869 is the system, tgRNA, or composition of any one of Embodiments 1-581, 628-669, and 695-868, wherein the guide RNA comprises a modification in a nexus region.

Embodiment 870 is the system, tgRNA, or composition of any one of Embodiments 1-581, 628-669, and 695-869, wherein the SpyCas9 guide RNA comprises a repeat/anti-repeat region modification, wherein the repeat/anti-repeat region modification comprises 2′-O-methyl (2′-O-Me) modifications at each nucleotide in the upper stem.

Embodiment 871 is the system, tgRNA, or composition of any one of Embodiments 1-581, 628-669, and 695-870, wherein the SpyCas9 guide RNA comprises a modified nucleotide sequence having at least 90% identity to any one of (a) SEQ ID NOs: 201-218, 224, 225-231, 232-235, 236, 237-239, 240-269, 270-276, 300-318, 323-325, 326-344, 345-349, and 372-374, 375-383, and 431-434, or any one of the sequences listed in Tables 1C and 1D, or a modified nucleotide sequence having at least 90%, 95%, 98% identity to any one of SEQ ID NOs: 350-374 and 384-400, 429-430, 435-454, or any one of the sequences listed in Table 4; or (b) SEQ ID NOs: 201-218, 224, 225-269, 270-271, 272-273, 274, 275 276, 300-318, 323-325, 326-344, 345-346, 347-349, 372-383, and 431-433, 434 or any one of the sequences listed in Tables 1C and 1D, or a modified nucleotide sequence having at least 90%, 95%, 98% identity to any one of SEQ ID NOs: 350-374, 384-400, and 429-430, and 435-454 or any one of the sequences listed in Table 4; optionally wherein the SpyCas9 guide RNA comprises a modified nucleotide sequence of any one of SEQ ID NOs: 201-218, 224, 225-250, 269, 272-273, 274, 275, 276, 301-318, 323-325, 326-334, 347-349, 372-374, and 431-433, 434.

Embodiment 872 is the system, tgRNA, or composition of any one of Embodiments 1-581, 628-669, and 695-870, wherein the SpyCas9 guide RNA comprises a modified nucleotide sequence of any one of SEQ ID NOs: 201-218, 224, 225-231, 232-235, 236, 237-239, 240-269, 270-273, 274, 275, 276, 300-318, 323-325, 326-344, 345-349,372-374, and 375-383 or any one of the sequences listed in Tables 1C and 1D or a modified nucleotide sequence of any one of SEQ ID NOs: 350-374 and 384-400, 429-430, 435-454 or any one of the sequences listed in Table 4; or (b) SEQ ID NOs: 201-218, 224, 225-269, 270-271, 272-273, 274, 275, 276, 300-318, 323-325, 326-344, 345-346, 347-349, 372-383, and 431-433, 434 or any one of the sequences listed in Tables 1C and 1D or a modified nucleotide sequence of any one of SEQ ID NOs: 350-374 and 384-400, and 435-454 or any one of the sequences listed in Table 4; optionally wherein the SpyCas9 guide RNA comprises a modified nucleotide sequence of any one of SEQ ID NOs: 201-218, 224, 225-250, 269, 272-273, 274, 275, 276, 301-318, 323-325, 326-334, 347-349, 372-374, and 431-433, 434.

Embodiment 873 is the system, tgRNA, or composition of any one of Embodiments 1-581, 628-669, and 695-870, wherein the SpyCas9 guide RNA comprises:
a) mN*mN*mN*NNNNNNNNNNNNNNNNNGUUUUAGA(L3)AAGUUAAAAUA AGGCUAGUCCGUUAUCAC(L1)GGGCACCGAGUCGGmU*mG*mC*mU (SEQ ID NO: 371) or a sequence having at least 90%, 95%, 98%, or 99% identity to SEQ ID NO: 371;
b) mN*mN*mN*NNNNNNNNNNNNNNNNNmGUUUUAGmAmGmC(L1)mGmCm AAGUUmAAmAAmUmAmAGmGCUmAGUCmCmGUUAUmCAmC(L1)mGGm GmCmAmCmCmGmAGUCmGmGmUmGmC (SEQ ID NO: 347) or a sequence having at least 90%, 95%, 98%, or 99% identity to SEQ ID NO: 347; or
c) mN*mN*mN*NNNNNNNNNNNNNNNNNGUUUUAGAmGmCmUmA(L1)mUm AmGmCAAGUUAAAAUAAGGCUAGUCCGUUAUCAC(L1)GGGCACCGAGU CGGmUmGmC (SEQ ID NO: 348) or a sequence having at least 90%, 95%, 98%, or 99% identity to SEQ ID NO: 348;
wherein * denotes that the nucleotide is linked to the next nucleotide with a PS bond, a lower case “m” denotes that the nucleotide is modified with 2′-O-Me, where N is any nucleotide.

Embodiment 874 is the system, tgRNA, or composition of any one of Embodiments 1-581, 628-669, and 695-870, wherein the SpyCas9 guide RNA comprises GUUUUAGAGCUAGAAAUAGCAAGUUAAAAUAAGGCUAGUCCGUUAUCAACUUGAAAAAG UGGCACCGAGUCGGUGCUUUU (SEQ ID NO: 4) or a sequence having at least 90%, 95%, 98%, or 99% identity to SEQ ID NO: 4.

Embodiment 875 is the system, tgRNA, or composition of any one of Embodiments 1-581, 628-669, and 695-870, wherein the SpyCas9 guide RNA comprises a sequence having at least 90%, 95%, 98%, 99% identity to SEQ ID NO: 209 or the sequence of SEQ ID NO: 209.

Embodiment 876 is the system, tgRNA, or composition of any one of Embodiments 1-581, 628-669 and 695-870, wherein the SpyCas9 guide RNA comprises GUUUUAGAGCUAGAAAUAGCAAGUUAAAAUAAGGCUAGUCCGUUAUCACGAAAGGGCAC CGAGUCGGUGC (SEQ ID NO: 27); or a sequence having at least 90%, 95%, 98%, or 99% identity to SEQ ID NO: 27.

Embodiment 877 is the system, tgRNA, or composition of any one of Embodiments 1-581 628-669 and 695-870, guide wherein the SpyCas9 RNA comprises GUUUUAGAGCUAGAAAUAGCAAGUUAAAAUAAGGCUAGUCCGUUAUCACGAAAGGGCAC CGAGUCGGUGCU (SEQ ID NO: 5) or a sequence having at least 90%, 95%, 98%, or 99% identity to SEQ ID NO: 5.

Embodiment 878 is the system, tgRNA, or composition of any one of Embodiments 1-581 628-669 and 695-870, wherein the SpyCas9 guide RNA comprises a sequence having at least 90%, 95%, 98%, 99% identity to SEQ ID NO: 210 or 269 or the sequence of SEQ ID NO: 210 or 269.

Embodiment 879 is the system, tgRNA, or composition of any one of Embodiments 1-581 628-669 and 695-870, wherein the SpyCas9 guide RNA comprises one or more of:
a) a shortened hairpin 1 region, or a substituted and optionally shortened hairpin 1 region, wherein
   1) at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks
      a. any one or two of H1-5 through H1-8,
      3. one, two, or three of the following pairs of nucleotides: H1-1 and H1-12, H1-2 and H1-11, H1-3 and H1-10, and H1-4 and H1-9, or
      4. 1-8 nucleotides of hairpin 1 region; or
   2) the shortened hairpin 1 region lacks 4-8 nucleotides, preferably 4-6 nucleotides; and
      1. one or more of positions H1-1, H1-2, or H1-3 is deleted or substituted relative to Exemplary SpyCas9 sgRNA-1 (SEQ ID NO: 22) or
      2. one or more of positions H1-6 through H1-10 is substituted relative to Exemplary SpyCas9 sgRNA-1 (SEQ ID NO: 22); or
   3) the shortened hairpin 1 region lacks 5-10 nucleotides, preferably 5-6 nucleotides, and one or more of positions N18, H1-12, or n is substituted relative to Exemplary SpyCas9 sgRNA-1 (SEQ ID NO: 22); or
b) a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1 (SEQ ID NO: 22); or
c) a substitution relative to Exemplary SpyCas9 sgRNA-1 (SEQ ID NO: 22) at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or
d) an Exemplary SpyCas9 sgRNA-1 (SEQ ID NO: 22) with an upper stem region, wherein the upper stem modification comprises a modification to any one or more of US1-US12 in the upper stem region.

Embodiment 880 is a guide RNA comprising, from 5′ to 3′, a guide region comprising 17-20 nucleotides, and
a) the sequence of GUUUUAGA(L3)AAGUUAAAAUAAGGCUAGUCCGUUAUCAC(L1)GGGCAC CGAGUCGGUGCU (SEQ ID NO: 370) or a sequence having at least 90%, 95%, 98%, 99% identity to SEQ ID NO: 370;
b) the sequence of GUUUUAGAGC(L1)GCAAGUUAAAAUAAGGCUAGUCCGUUAUCAC(L1) GG GCACCGAGUCGGUGC (SEQ ID NO: 429) or a sequence having at least 90%, 95%, 98%, 99% identity to SEQ ID NO: 429; or
c) the sequence of GUUUUAGAGCUA(L1)UAGCAAGUUAAAAUAAGGCUAGUCCGUUAUCAC(L1)GGGCACCGAGUCGGUGC (SEQ ID NO: 430) or a sequence having at least 90%, 95%, 98%, 99% identity to SEQ ID NO: 430,
wherein L1 denotes that the linker is S18, and L3 denotes that the linker is S6.

Embodiment 881 is a lipid nanoparticle (LNP) composition comprising the system, tgRNA, guide RNA, or composition of any one of Embodiments 1-581, 628-669, and 695-880.

Embodiment 882 is the system, tgRNA, guide RNA, composition, or LNP composition of any one of Embodiments 1-581, 628-669, and 695-881, wherein one or more of the tgRNA, first polynucleotide, second polynucleotide, nucleic acid encoding the DNA-dependent DNA polymerase, nucleic acid encoding the nCas9, or nucleic acid encoding the fusion protein is associated with one or more LNP.

Embodiment 883 is the system, tgRNA, guide RNA, composition, or LNP composition of any one of Embodiments 1-581, 628-669, and 695-882, wherein the tgRNA and nucleic acid encoding the DNA-dependent DNA polymerase and nCas9 are each associated with a separate lipid nanoparticle.

Embodiment 884 is the system, tgRNA, guide RNA, composition, or LNP composition of any one of Embodiments 880-883, wherein the tgRNA and nucleic acid encoding the DNA-dependent DNA polymerase and nCas9 are associated with the same LNP.

Embodiment 885 is the system, tgRNA, guide RNA, composition, or LNP composition of any one of Embodiments 1-581, 628-669, and 695-884, wherein the DNA-dependent DNA polymerase and nCas9 are a fusion protein encoded by a single mRNA associated with one LNP.

Embodiment 886 is the system, tgRNA, guide RNA, composition, or LNP composition of Embodiment 885, wherein the tgRNA is associated with a separate LNP from the LNP associated with the mRNA encoding the DNA-dependent DNA polymerase and nCas9 are a fusion protein.

Embodiment 887 is the system, tgRNA, guide RNA, composition, or LNP composition of Embodiment 885 or 886, wherein the system or composition further comprises an mRNA encoding an enhancer of template-based genome editing associated with an LNP.

Embodiment 888 is the system, tgRNA, guide RNA, composition, or LNP composition of Embodiment 887, wherein the mRNA encoding an enhancer of template based genome editing is associated with a separate LNP.

Embodiment 889 is the system, tgRNA, guide RNA, composition, or LNP composition of any one of Embodiments 881-889, wherein the LNP comprises (i) an ionizable lipid; (ii) a helper lipid; (iii) a stealth lipid; (iv) a neutral lipid; or combinations of one or more of (i)-(iv).

Embodiment 890 is the system, tgRNA, guide RNA, composition, or LNP composition of Embodiment 889, wherein the ionizable lipid is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate.

Embodiment 891 is the system, tgRNA, guide RNA, composition, or LNP composition of Embodiment 889 or 890, wherein the helper lipid is cholesterol.

Embodiment 892 is the system, tgRNA, guide RNA, composition, or LNP of any one of Embodiments 889-891, wherein the stealth lipid is PEG-DMG.

Embodiment 893 is the system, tgRNA, guide RNA, composition, or LNP composition of Embodiment 892, wherein the PEG-DMG is 1,2-dimyristoyl-rac-glycero-3-methoxypolyethylene glycol-2000 (PEG2k-DMG).

Embodiment 894 is the system, tgRNA, guide RNA, composition, or LNP composition of any one of Embodiments 889-893, wherein the neutral lipid is DSPC.

Embodiment 895 is the system, tgRNA, guide RNA, composition, or LNP composition of any one of Embodiments 881-894 wherein the LNP composition comprises about 50 mol-% ionizable lipid; about 9 mol-% neutral lipid; about 3 mol-% of stealth lipid, and the remainder of the lipid component is helper lipid such as cholesterol.

Embodiment 896 is the system, tgRNA, guide RNA, composition, or LNP composition of any one of Embodiments 881-895, wherein the LNP comprises (9Z,12Z)-3-((4,4-bis(octyloxy)butanoyl)oxy)-2-((((3-(diethylamino)propoxy)carbonyl)oxy)methyl)propyl octadeca-9, 12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate, DSPC, cholesterol, and PEG2k-DMG.

Embodiment 897 is a pharmaceutical composition comprising the system, tgRNA, guide RNA, composition, fusion polypeptide, or LNP composition of any one of Embodiments 1-581, 628-669, and 695-896 and a pharmaceutically acceptable carrier.

Embodiment 898 is a method for template-based genomic editing, comprising delivering to a cell or subject the system, tgRNA, guide RNA, composition, or LNP composition of any one of Embodiments 1-581, 628-669, and 695-896 or the fusion polypeptide of any one of Embodiments 208-252.

Embodiment 899 is the use of the system, tgRNA, guide RNA, composition, or LNP composition of any one of Embodiments 1-581, 628-669, and 695-896 or the fusion polypeptide of any one of Embodiments 208-252 for template-based genome editing in a cell or subject.

Embodiment 900 is the method or use of Embodiment 898 or 899, wherein template-based genome editing results in edits comprising an insertion or deletion in a nucleotide sequence.

Embodiment 901 is the method or use of Embodiment 898 or 899, wherein template-based genome editing results in edits comprising a substitution in a nucleotide sequence.

Embodiment 902 is the method or use of Embodiment 901, wherein the substitution is a transition or transversion.

Embodiment 903 is the method or use of any one of Embodiments 898-902 wherein template-based genome editing results in at least 20%, 25%, 30%, 35%, 40%, 45%, optionally at least 50%, 55%, 60%, 65%, optionally at least 70%, 75%, or 80% templated edits as a percent of total edits in the target nucleic acid.

Embodiment 904 is the method or use of any one of Embodiments 898-903, wherein the cell is a quiescent cell and wherein at least 5% of the cells in the cell population comprise a template-based genome edit in the target nucleic acid.

Embodiment 905 is the method or use of Embodiment 904, wherein the quiescent cell is selected from a hepatocyte, unactivated T-cell, muscle cell, and neuronal cell.

Embodiment 906 is the method or use of Embodiment 905, wherein the hepatocyte, muscle cell, or neuronal cell is in vivo.

Embodiment 907 is the method or use of any one of Embodiments 898-906, wherein the cell is a primary cell.

Embodiment 908 is the method or use of any one of Embodiments 898-907, wherein the cell is an immune cell and wherein at least 50% of the cells in the cell population comprise a template-based genome edit in the target nucleic acid.

Embodiment 909 is the method or use of any one of Embodiments 898-908, wherein the length of the templated insertion is at least 5, 10, 15, 20, 25, 30, 35, or 40 nucleotides in length.

Embodiment 910 is the method or use of any one of Embodiments 898-909, wherein the length of the templated deletion is 1-10,000 consecutive nucleotides.

Embodiment 911 is the method or use of any one of Embodiments 898-910, wherein the system further comprises a nicking guide RNA (ngRNA) or the method further comprises delivering the ngRNA to a cell or subject.

Embodiment 912 is the method or use of Embodiment 911, wherein the ngRNA comprises a spacer that is complementary to the non-target strand of the DNA duplex target nucleic acid.

Embodiment 913 is the method or use of Embodiment 911 or 912, wherein the ngRNA comprises a spacer that hybridizes to a sequence such that the nick site of the ngRNA is within 200 nucleotides 5′ or 3′ from the nick site of the template guide RNA and on the target strand of the DNA duplex target nucleic acid.

Embodiment 914 is the method or use of Embodiment 911 or 912, wherein the nick site of the ngRNA is about 20-200 nucleotides 5′ or 3′ from the nick site of the template guide RNA and on the target strand of the DNA duplex target nucleic acid.

Embodiment 915 is the method or use of Embodiment 911 or 912, wherein the nick site of the ngRNA is within the genomic locus complementary to the spacer sequence of the template guide RNA.

Embodiment 916 is the method or use of Embodiment 911 or 912, wherein the nick site of the ngRNA is outside of the genomic locus complementary to the spacer sequence of the template guide RNA.

Embodiment 917 is a method of producing the template guide RNA (tgRNA) of any one of Embodiments 114-247, the method comprising:
a) providing a first polynucleotide comprising a 3′ end comprising at least 10 nucleotides;
b) providing a second polynucleotide comprising a 5′ end comprising at least 10 nucleotides;
c) providing a splint oligonucleotide capable of annealing to the at least 10 nucleotides at the 3′ end of the first polynucleotide and the at least 10 nucleotides at the 5′ end of the second polynucleotide; and
d) providing a ligase to ligate the 3′ end of the first polynucleotide to the 5′ end of the second polynucleotide thereby forming a ligated tgRNA.

Embodiment 918 is a method of producing the template guide RNA (tgRNA) of any one of Embodiments 114-247, the method comprising:
a) providing a first polynucleotide comprising a 3′ end comprising at least 10 nucleotides;
b) providing a second polynucleotide comprising a 5′ end comprising at least 10 nucleotides and a 3′ end comprising at least 10 nucleotides;
c) providing a third polynucleotide comprising a 5′ end comprising at least 10 nucleotides;
d) providing a first splint oligonucleotide capable of annealing to the at least 10 nucleotides at the 3′ end of the first polynucleotide and the at least 10 nucleotides at the 5′ end of the second polynucleotide;
e) providing a second splint oligonucleotide capable of annealing to the at least 10 nucleotides at the 3′ end of the second polynucleotide and the at least 10 nucleotides at the 5′ end of the third polynucleotide; and
f) providing one or more ligases to ligate (i) the 3′ end of the first polynucleotide to the 5′ end of the second polynucleotide and (ii) the 3′ end of the second polynucleotide to the 5′ end of the third polynucleotide thereby forming a ligated tgRNA.

Embodiment 919 is a method of producing a ligated template guide RNA (tgRNA), the method comprising:
a) providing a first polynucleotide comprising a 3′ end comprising at least 10 nucleotides;
b) providing a second polynucleotide comprising a 5′ end comprising at least 10 nucleotides;
c) providing a splint oligonucleotide capable of annealing to the at least 10 nucleotides at the 3′ end of the first polynucleotide and the at least 10 nucleotides at the 5′ end of the second polynucleotide; and
d) providing a ligase to ligate the 3′ end of the first polynucleotide to the 5′ end of the second polynucleotide thereby forming a ligated tgRNA;
wherein the ligated tgRNA comprises a SpyCas9 guide RNA, a template sequence, and a DNA-dependent DNA polymerase recruiting sequence (DRS).

Embodiment 920 is a method of producing a ligated template guide RNA (tgRNA), the method comprising:
a) providing a first polynucleotide comprising a 3′ end comprising at least 10 nucleotides; providing a second polynucleotide comprising a 5′ end comprising at least 10 nucleotides and a 3′ end comprising at least 10 nucleotides;
b) providing a third polynucleotide comprising a 5′ end comprising at least 10 nucleotides; providing a first splint oligonucleotide capable of annealing to the at least 10 nucleotides at the 3′ end of the first polynucleotide and the at least 10 nucleotides at the 5′ end of the second polynucleotide;
c) providing a second splint oligonucleotide capable of annealing to the at least 10 nucleotides at the 3′ end of the second polynucleotide and the at least 10 nucleotides at the 5′ end of the third polynucleotide; and
d) providing one or more ligases to ligate (i) the 3′ end of the first polynucleotide to the 5′ end of the second polynucleotide and (ii) the 3′ end of the second polynucleotide to the 5′ end of the third polynucleotide thereby forming a ligated tgRNA;
wherein the ligated tgRNA comprises a SpyCas9 guide RNA, a template sequence, and a DNA-dependent DNA polymerase recruiting sequence (DRS).

Embodiment 921 is the method of Embodiment 919 or 920, wherein the SpyCas9 gRNA comprises i) a spacer that is complementary to a target region of a target strand of a DNA duplex target nucleic acid; and ii) a scaffold.

Embodiment 922 is the method of Embodiment 919 or 920, wherein the template sequence is at least 10 nucleotides in length and complementary to at least a portion of a non-target strand of the DNA duplex target nucleic acid, wherein the template sequence comprises a 3′ end and a 5′ end; and wherein the template comprises DNA nucleotide.

Embodiment 923 is the method of Embodiment 919 or 920, wherein the DRS is 6-18 nucleotides in length, optionally 8-18 or 10-12 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the nontarget strand of the DNA duplex target nucleic acid, wherein the DRS comprises RNA nucleotides.

Embodiment 924 is the method of Embodiment 919 or 920, wherein the ligated tgRNA comprises in order from 5′ to 3′ the SpyCas9 guide RNA, the template sequence, and the DNA-dependent DNA polymerase recruiting sequence (DRS)

Embodiment 925 is the method of any one of Embodiments 919-924, wherein the ligated tgRNA is at least 100 nucleotides in length, at least 110 nucleotides in length, at least 120 nucleotides in length, at least 130 nucleotides in length, at least 140 nucleotides in length, at least 150 nucleotides in length, at least 160 nucleotides in length, or at least 170 nucleotides in length.

Embodiment 926 is the method of any one of Embodiments 919-925, further comprising separating the ligated tgRNA from the splint oligonucleotide.

Embodiment 927 is the method of Embodiment 926, wherein the separating is achieved by chromatography.

Embodiment 928 is the method of any one of Embodiments 919-927, further comprising subjecting the ligated tgRNA to a purification step.

Embodiment 929 is the method of Embodiment 928, wherein the purification steps comprises subjecting the ligated tgRNA to high-performance liquid chromatography (HPLC).

Embodiment 930 is the method of any one of Embodiments 919-929, wherein the 5′ end of the second polynucleotide comprises a 5′ phosphate moiety.

Embodiment 931 is the method of any one of Embodiments 919-930, wherein the splint oligonucleotide is a DNA splint oligonucleotide.

Embodiment 932 is the method of any one of Embodiments 919-931, wherein the splint oligonucleotide is at least 20 nucleotides in length.

Embodiment 933 is the method of any one of Embodiments 919-932, wherein the ligase is an RNA ligase and wherein ligation occurs at a ligation site flanked by RNA nucleotides.

Embodiment 934 is the method of any one of Embodiments 919-933, wherein the ligase is a DNA ligase and wherein ligation occurs at a ligation site that is flanked by DNA nucleotides.

Embodiment 935 is the method of any one of Embodiments 919-934, wherein ligation occurs at a ligation site within the scaffold of the SpyCas9 guide RNA.

Embodiment 936 is the method of Embodiment 935, wherein the ligation site within the scaffold of the SpyCas9 guide RNA is within the hairpin 2 region.

Embodiment 937 is the method of any one of Embodiments 919-936, further comprising separating the ligated tgRNA from the first and second splint oligonucleotides.

Embodiment 938 is the method of Embodiment 937, wherein the separating is achieved by chromatography.

Embodiment 939 is the method of any one of Embodiments 919-938, further comprising subjecting the ligated tgRNA to a purification step.

Embodiment 940 is the method of Embodiment 939, wherein the purification step comprises subjecting the ligated tgRNA to high-performance liquid chromatography (HPLC).

Embodiment 941 is the method of any one of Embodiments 919-940, wherein the 5′ ends of both the second polynucleotide and the third polynucleotide comprise a 5′ phosphate moiety.

Embodiment 942 is the method of any one of Embodiments 919-941, wherein the first and second splint oligonucleotides are DNA splint oligonucleotides.

Embodiment 943 is the method of any one of Embodiments 919-942, wherein the first splint oligonucleotide is at least 20 nucleotides in length.

Embodiment 944 is the method of any one of Embodiments 919-943, wherein the second splint oligonucleotide is at least 20, 30, 40, 50, 60, 70, 80, or 90 nucleotides in length.

Embodiment 945 is the method of any one of Embodiments 919-944, wherein the one or more ligases comprises an RNA ligase or a DNA ligase.

Embodiment 946 is the method of any one of Embodiments 919-944, wherein the one or more ligases comprises an RNA ligase and a DNA ligase.

Embodiment 947 is the method of any one of Embodiments 919-946, wherein the one or more ligases is selected from a T4 DNA ligase, T4 RNA ligase 1, T4 RNA ligase 2, RtcB ligase, T3 DNA ligase, T7 DNA ligase, Taq DNA ligase, PBCV-1 DNA ligase, thermostable DNA ligase (e.g., 5′AppDNA/RNA ligase), an ATP dependent DNA ligase, or SplintR ligase.

Embodiment 948 is the method of any one of Embodiments 919-945 and 947, wherein the ligase comprises a T4 DNA ligase or a T4 RNA ligase.

Embodiment 949 is the method of any one of Embodiments 919-948, wherein a first ligation occurs at a first ligation site that is flanked by RNA nucleotides and a second ligation occurs at a second ligation site that is flanked by DNA nucleotides.

Embodiment 950 is the method of any one of Embodiments 919-948, wherein a first ligation occurs at a first ligation site within the scaffold of the SpyCas9 guide RNA.

Embodiment 951 is the method of Embodiment 950, wherein the first ligation site within the scaffold of the SpyCas9 guide RNA is within the nexus region.

Embodiment 952 is the method of any one of Embodiments 919-951, wherein a second ligation occurs at a second ligation site within the template region of the tgRNA.

LENGTHY TABLES
The patent application contains a lengthy table section. A copy of the table is available in electronic form from the USPTO web site (https://seqdata.uspto.gov/docdetail?docId=US20260286329A1). An electronic copy of the table will also be available from the USPTO upon request and payment of the fee set forth in 37 CFR 1.19(b)(3).

We claim:

1. A system comprising a DNA-dependent DNA polymerase, a SpyCas9 nickase (nCas9), and a template guide RNA (tgRNA), wherein the tgRNA comprises a first polynucleotide and a second polynucleotide, wherein:
a) the first polynucleotide comprises a SpyCas9 guide RNA comprising, from 5′ to 3′:
i) a spacer that is complementary to a target region of a target strand of a DNA duplex target nucleic acid; and
ii) a scaffold; and
b) the second polynucleotide is operably linked to the first polynucleotide and comprises, from 5′ to 3′:
i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; and
ii) a DNA-dependent DNA polymerase recruiting sequence (DRS) that is 6-20 nucleotides in length, optionally 10-14 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid, wherein the DRS comprises RNA nucleotides; and
iii) wherein the DRS comprises at least one of:
1) a sequence not fully complementary to the DRS-complementary region of the non-target strand of the DNA duplex target nucleic acid;
2) a 1-5 nucleotide, optionally a 1-3 nucleotide 3′ terminal tail comprising a 3′ terminal modified uridine nucleotide; or
3) a 5′ terminal (D1) DNA nucleotide and a penultimate 5′ terminal (D2) RNA nucleotide wherein the 3′ end of the first polynucleotide is covalently linked to the 5′ end of the second polynucleotide by a phosphorothioate or phosphodiester linkage.

2. The system of claim 1, wherein the first polynucleotide is covalently linked to the second polynucleotide, optionally wherein the 3′ end of the first polynucleotide is covalently linked to the 5′ end of the second polynucleotide.

3. The system of any one of claims 1-2, wherein the first polynucleotide is linked to the second polynucleotide by a non-nucleotide linker, optionally a phosphodiester bond or a phosphorothioate bond.

4. The system of any one of claims 1-3, wherein the 3′ terminal nucleotide of the template is a DNA nucleotide.

5. The system of any one of claims 1-4, wherein (i) the 5′ terminal nucleotide of the DRS is a DNA nucleotide; or (ii) the penultimate 5′ terminal nucleotide of the DRS is an RNA nucleotide.

6. The system of any one of claims 1-5, wherein the 3′ terminal nucleotide of the template and the 5′ terminal nucleotide of the DRS are DNA nucleotides, and the penultimate 5′ terminal nucleotide of the DRS is an RNA nucleotide.

7. The system of any one of claims 1-4, wherein the 3′ terminal nucleotide in the DRS is an RNA nucleotide and the remaining nucleotides in the DRS are DNA nucleotides.

8. The system of any one of claims 1-7, wherein the template consists of DNA nucleotides.

9. The system of any one of claims 1-4, wherein the DRS consists of RNA nucleotides.

10. The system of any one of claims 1-9, wherein (i) the sequence of the DRS that is complementary to the DRS-complementary region of the non-target strand of the DNA duplex target nucleic acid comprises nucleotides D2-D9 of the DRS, optionally D2-D8 or D3-D9 of the DRS; or (ii) the sequence of the DRS that is complementary to a DRS-complementary region of a non-target strand of the DNA duplex target nucleic acid comprises nucleotides D6-D13 of the DRS, optionally wherein 1, 2, 3, 4, 5, 6, 7, or 8 of nucleotides D6-D13 are DNA nucleotides.

11. The system of claim 1, wherein the tgRNA further comprises an affinity tag, wherein the first polynucleotide or the second polynucleotide are bound to a peptide or a peptide complex comprising the nCas9 and the DNA-dependent DNA polymerase by the affinity tag.

12. The system of claim 11, wherein 1-7 consecutive 5′ terminal nucleotides of the DRS are DNA nucleotides, optionally wherein:
i) the 5′ terminal and penultimate 5′ terminal nucleotides of the DRS are DNA nucleotides;
ii) at least 3 consecutive 5′ terminal nucleotides of the DRS are DNA nucleotides; or
iii) 3 consecutive 5′ terminal nucleotides of the DRS are DNA nucleotides.

13. The system of any one of claims 11-12, wherein the affinity tag is located 5′ of the template sequence or the affinity tag is located 3′ of the DRS.

14. The system of any one of claims 11-13, wherein the tgRNA comprises two affinity tags, optionally wherein (i) both affinity tags are located 5′ of the template; or (ii) a first affinity tag is located 5′ of the template sequence and a second affinity tag is located 3′ of the DRS, wherein the DNA-dependent DNA polymerase is T5 DNA polymerase.

15. The system of any one of claims 11-14, wherein the affinity tag is an aptamer comprising one or more modified nucleotides, optionally wherein the aptamer is located 5′ of the template and has a 5′ terminus, further optionally wherein the aptamer comprises one or more modified nucleotide at the 5′ end thereof.

16. The system of claim 15, wherein the modified nucleotide is a 2′-O-methyl (2′-O-Me) modified nucleotide or a phosphorothioate (PS) linkage modified nucleotide or wherein the aptamer comprises a stem-loop, optionally wherein the aptamer comprises 2′-O-Me modified nucleotides at each nucleotide in the stem of the stem-loop.

17. The system of any one of claim 15 or 16, wherein the aptamer is located 5′ of the template sequence, further wherein:
i) the aptamer comprises a PS linkage between each of the first three nucleotides at the 5′ end;
ii) the aptamer comprises 2′-O-Me modified nucleotides at each of the first two nucleotides at the 5′ end;
iii) the aptamer comprises 2′-O-Me modified nucleotides at each of the first two nucleotides at the 5′ end and a PS linkage between each of the first three nucleotides at the 5′ end; or
iv) the aptamer comprises 2′-O-Me modified nucleotides at each nucleotide in the stem of the stem-loop and a PS linkage between each of the first three nucleotides at the 5′ end.

18. The system of any one of claims 15-17, wherein the aptamer is located 3′ of the DRS, further wherein: the affinity tag comprises a PS linkage between the last two nucleotides at the 3′ end; or the aptamer comprises 2′-O-Me modified nucleotides at each nucleotide in the stem of the stem-loop and a PS linkage between the last two nucleotides at the 3′ end.

19. The system of any one of claims 15-18, wherein the aptamer comprises 2′-O-Me modified nucleotides at each nucleotide.

20. The system of any one of claims 1-19, wherein the DRS comprises a modified nucleotide.

21. The system of any one of claims 1-20, wherein the DRS comprises a modified nucleotide selected from:
i) 2′-O-methyl (2′-O-Me) modified nucleotide;
ii) 2′-fluoro (2′-F) modified nucleotide;
iii) an amine modified nucleotide;
iv) an alkoxy modified nucleotide;
v) an LNA modified nucleotide;
vi) methoxyethyl modified nucleotide;
vii) a halo modified nucleotide; or
viii) phosphorothioate (PS) linkage modified nucleotide;
optionally, wherein the modified nucleotide is a 2′-O-methyl (2′-O-Me) modified nucleotide or a phosphorothioate (PS) linkage modified nucleotide.

22. The system of claim 20 or 21, wherein:
i) the modification in the DRS comprises a phosphorothioate modification, and the DRS does not comprise a phosphorothioate modification at position D1-D9 in the DRS sequence;
ii) the modification in the DRS comprises a phosphorothioate modification, and the DRS comprises a phosphorothioate modification at or 3′ of position D10 in the DRS sequence;
iii) the modification in the DRS comprises a 2′-O-Me modification, and the DRS does not comprise a 2′-O-Me modification at position D1-D3 in the DRS sequence;
iv) the modification in the DRS comprises a 2′-O-Me modification, and the DRS comprises a 2′-O-Me modification at or 3′ of position D4 in the DRS sequence; or
v) the DRS comprises a 2′-O-Me modification at a nucleotide other than the 5′ terminal and 3′ terminal nucleotides of the DRS.

23. The system of any one of claims 1-22, wherein the DNA-dependent DNA polymerase and nCas9 are provided as one or more polypeptides.

24. The system of any one of claims 1-22, wherein the DNA-dependent DNA polymerase and nCas9 are provided as one or more polynucleotides.

25. The system of any one of claims 1-24, wherein the DNA-dependent DNA polymerase comprises DNA polymerase K (polK), optionally human DNA polymerase K (polK), cynomolgus polK, or mouse polK, phage T5 DNA polymerase (T5 pol), DNA polymerase θ, E. coli DNA polymerase I, optionally wherein the E. coli dependent DNA polymerase is Klenow (exo-), or DNA polymerase N, optionally wherein the DNA-dependent DNA polymerase is polK or T5 DNA polymerase.

26. The system of claims 1-25, comprising an nCas9-polymerase fusion polypeptide.

27. The system of claim 26, wherein the fusion polypeptide further comprises (i) a heterologous nuclear localization signal (NLS); or (ii) a linker or further comprises an additional linker.

28. The system of claim 26 or 27, wherein the polymerase is selected from: polymerase θ, E. coli polymerase I, optionally an E. coli polymerase I Klenow (exo-), polymerase N, or Phi29 polymerase, wherein:
i) the polymerase Θ and nCas9 are covalently linked sequentially in a polypeptide from N-terminus to C-terminus in an order selected from:
1) nCas9-polymerase Q-NLS; or
2) nCas9-linker-polymerase Q-linker-NLS; or
ii) the E. coli polymerase I and nCas9 are covalently linked sequentially in a polypeptide from N-terminus to C-terminus in an order selected from:
1) nCas9-Klenow (exo-)-NLS;
2) nCas9-linker-Klenow (exo-)-linker-NLS;
3) NLS-nCas9-NLS-E coli PolI; or
4) NLS-nCas9-linker-NLS-linker-E coli PolI; or
iii) the polymerase N and nCas9 are covalently linked sequentially in a polypeptide from N-terminus to C-terminus in an order selected from:
1) polymerase N-NLS-nCas9-NLS; or
2) polymerase N-linker-NLS-nCas9-linker-NLS; or
iv) the Phi29 polymerase and nCas9 are covalently linked sequentially in a polypeptide from N-terminus to C-terminus in an order selected from:
1) NLS-nCas9-NLS-Phi29 polymerase;
2) ii. NLS-nCas9-linker-NLS-linker-Phi29 polymerase;
3) nCas9-Phi29 polymerase-NLS; or
4) nCas9-linker-Phi29 polymerase-linker-NLS.

29. The system of claim 27 or 28, wherein, the linker comprises an amino acid sequence of any one of SEQ ID NOs: 1596-1665, 1735, 1738, 1815, 1825, 1827, and 1829-1830.

30. The system of any one of claims 26-29, wherein the fusion polypeptide comprises an amino acid sequence that has at least 90%, 91%, 92%, 93%, 94%, 95%, 96%, 97%, 98%, 99%, or 100% identity to any one of SEQ ID NOs: 1024, 1027, 1030, 1033, 1036, 1039, 1042, 1045, 1048, 1053, 1056, 1059, 1074, 1081, 1084, 1086, 1094, 1097, 1100, 1102, 1112, 1114, 1124, 1129, 1134, 1137, 1142, 1147, 1152, 1157, 1162, 1167, 1172, 1192, 1195, 1198, 1219, 1224, 1229, 1234, 1236, 1239, 1254, and 1259, 1268, and 1273, 1276, 1300, 1303, 1307, 1373, 1376, 1388, 1447, 1450, 1453, 1923, 1926, 1929, 1932, 1935, and 1938, or a nucleic acid encoding the fusion protein sequence of any one of SEQ ID NOs: 1024, 1027, 1030, 1033, 1036, 1039, 1042, 1045, 1048, 1053, 1056, 1059, 1074, 1081, 1084, 1086, 1094, 1097, 1100, 1102, 1112, 1114, 1124, 1129, 1134, 1137, 1142, 1147, 1152, 1157, 1162, 1167, 1172, 1192, 1195, 1198, 1219, 1224, 1229, 1234, 1236, 1239, 1254, 1259, 1268, and 1273, 1276, 1300, 1303, 1307, 1373, 1376, 1388, 1477, 1450, 1453, 1923, 1926, 1929, 1932, 1935, and 1938.

31. The system of any one of claims 1-30, wherein the DNA-dependent DNA polymerase is (i) a human DNA-dependent DNA polymerase; or (ii) a cynomolgus or mouse DNA-dependent DNA polymerase.

32. The system of any one of claims 1-31, wherein the DNA-dependent DNA polymerase is not a Phi29 DNA polymerase.

33. The system of any one of claims 1-32, wherein the DNA-dependent DNA polymerase does not comprise at least 8 point mutations, optionally wherein the DNA-dependent DNA polymerase does not comprise at least 5 point mutations.

34. The system of any one of claims 1-33, wherein the wild-type DNA-dependent DNA polymerase has a polymerization rate at least twice as high, optionally at least five times as high at 37° C. as compared to 30° C.

35. The system of any one of claims 1-34, comprising:
i) a DNA polymerase K (polK), comprising an amino acid sequence that has at least 90%, 91%, 92%, 93%, 94%, 95%, 96%, 97%, 98%, 99%, or 100% identity to amino acid residues 19-526 of SEQ ID NO: 1021; or an amino acid sequence that has at least 90%, 91%, 92%, 93%, 94%, 95%, 96%, 97%, 98%, 99%, or 100% identity to SEQ ID NO: 1021 or 1206, optionally wherein the polk includes or lacks an N-terminal methionine relative to SEQ ID NO: 1021 or 1206; or a nucleic acid encoding the polk, comprising a nucleotide sequence that has at least 90%, 91%, 92%, 93%, 94%, 95%, 96%, 97%, 98%, 99%, or 100% identity to SEQ ID NO: 1020 or 1205, optionally wherein the nucleotide sequence includes or lacks a start or stop codon relative to SEQ ID NO: 1020 or 1205; or
ii) a T5 polymerase (T5 pol) comprising an amino acid sequence that has at least 90%, 91%, 92%, 93%, 94%, 95%, 96%, 97%, 98%, 99%, or 100% identity to any one of SEQ ID NO:
1116 or 1121, optionally wherein the T5 pol includes or lacks an N-terminal methionine relative to SEQ ID NO: 1116 or 1121; or a nucleic acid encoding the T5, comprising a nucleotide sequence that has at least 90%, 91%, 92%, 93%, 94%, 95%, 96%, 97%, 98%, 99%, or 100% identity to SEQ ID NO:1115 or 1120, optionally wherein the nucleotide sequence includes or lacks a start or stop codon relative to SEQ ID NO: 1115 or 1120.

36. The system of any one of claims 1-35, wherein the DNA-dependent DNA polymerase comprises a heterodimerization domain or further comprises an additional heterodimerization domain, optionally wherein the DNA-dependent DNA polymerase and nCas9 are covalently linked sequentially in a polypeptide from N-terminus to C-terminus in an order of:
i) NLS-nCas9-linker-NLS-linker-DNA-dependent DNA polymerase-NLS-NLS-linker-heterodimerization domain; or
ii) NLS-nCas9-linker-NLS-linker-DNA-dependent DNA polymerase-linker-NLS-linker-heterodimerization domain.

37. The system of claim 36, wherein the heterodimerization domain comprises a coiled coil heterodimerization domain, optionally an EV, EI, KV, or KI domain.

38. The system of any one of claim 36 or 37, wherein the heterodimerization domain comprises an EI or KI domain, optionally wherein the heterodimerization domain comprises SEQ ID NO: 1589, SEQ ID NO: 1593, or SEQ ID NO: 1595.

39. The system of any one of claims 1-25 and 31-38, comprising:
a) a first polypeptide comprising the DNA-dependent DNA polymerase operably linked to a first split-intein, or a nucleic acid encoding the first polypeptide; and (ii) a second polypeptide comprising the nCas9 operably linked to a second split-intein that is complementary to the first split intein, or a nucleic acid encoding the second polypeptide;
b) a first polypeptide comprising a first portion of the DNA-dependent DNA polymerase operably linked to a first split-intein, or a nucleic acid encoding the first polypeptide; and (ii) a second polypeptide comprising a second portion of the DNA-dependent DNA polymerase and an nCas9 operably linked to a second split-intein that is complementary to the first split-intein; or
c) a first polypeptide comprising a DNA-dependent DNA polymerase and a first portion of an nCas9 operably linked to a first split-intein, or a nucleic acid encoding the first polypeptide; and (ii) a second polypeptide comprising a second portion of the nCas9 operably linked to a second split-intein that is complementary to the first split-intein, or a nucleic acid encoding the second polypeptide,
optionally wherein the first split-intein and the second split-intein are capable of binding to each other to undergo intein-mediated protein splicing.

40. The system of claim 39, wherein the first split-intein is an N-split-intein and the second split-intein is a C-split-intein, or wherein the first split-intein is a C-split-intein and the second split intein is an N-split-intein.

41. The system of any one of claims 39-40, wherein the first split-intein and second split-intein are complementary inteins selected from Cfa, M86, Npu, NpuSsp, gp41-1, gp41-8, NrdJ-1, IMPDH-1, SspDnaX, SspGyrB, Cth-Ter, NrdA-2, Mja-KlbA, RBS1, TE3S11, and Rma, optionally wherein the first split-intein and second split-intein are Cfa-inteins.

42. The system of claim 41, wherein the first split-intein is a CfaC split-intein and the second split-intein is a CfaN split-intein, optionally wherein (i) the CfaC split-intein comprises an amino acid sequence having at least 90% identity to SEQ ID NO: 1746 and the CfaN split-intein comprises an amino acid sequence having at least 90% identity to SEQ ID NO: 1744; or (ii) the CfaC split-intein comprises the amino acid sequence of SEQ ID NO: 1746 and the CfaN split-intein comprises the amino acid sequence of SEQ ID NO: 1744.

43. The system of any one of claims 39-42, wherein the DNA-dependent DNA polymerase or the first portion of the DNA-dependent DNA polymerase is operably linked to the first split-intein by a first peptide linker, optionally wherein the first peptide linker comprises 30 amino acids, optionally wherein the first peptide linker comprises an amino acids sequence at least 90%, 95%, 98%, or 100% identical to SEQ ID NO: 1829.

44. The system of any one of claims 39-43, wherein the nCas9 or second portion of the nCas9 is operably linked to the second split-intein by a second peptide linker, optionally wherein the second peptide linker is SGGS (SEQ ID NO: 1738).

45. The system of any one of claims 39-44, wherein (i) the DNA-dependent DNA polymerase or the first portion of the DNA-dependent DNA polymerase is operably linked to a heterologous nuclear localization signal (NLS); or (ii) the nCas9 or the second portion of the nCas9 is operably linked to a heterologous nuclear localization signal (NLS); optionally wherein the heterologous NLS is selected from an SV40 NLS, SV40-c-Myc, and nucleoplasmin.

46. The system of any one of claims 1-45, further comprising at least one chromatin remodeler.

47. The system of claim 46, wherein the DNA-dependent DNA polymerase, nCas9, and chromatin remodeler are covalently linked sequentially in a polypeptide from N-terminus to C-terminus in an order selected from:
i) chromatin remodeler-nCas9-DNA-dependent DNA polymerase;
ii) nCas9-chromatin remodeler-DNA-dependent DNA polymerase; or
iii) nCas9-DNA-dependent DNA polymerase-chromatin remodeler.

48. The system of claim 47, further comprising a nuclear localization signal (NLS), optionally wherein the DNA-dependent DNA polymerase, nCas9, and chromatin remodeler are covalently linked sequentially in a polypeptide from N-terminus to C-terminus in an order selected from:
i) NLS-chromatin remodeler-nCas9-DNA-dependent DNA polymerase-NLS;
ii) NLS-chromatin remodeler-nCas9-NLS-DNA-dependent DNA polymerase-NLS;
iii) NLS-nCas9-chromatin remodeler-DNA-dependent DNA polymerase-NLS; or
iv) NLS-nCas9-chromatin remodeler-NLS-DNA-dependent DNA polymerase-NLS;
nCas9-DNA-dependent DNA polymerase-chromatin remodeler-NLS.

49. The system of any one of claims 46-48, further comprising one or more linkers between the NLS, nCas9, DNA-dependent DNA polymerase, and the chromatin remodeler.

50. The system of any one of claims 46-49, wherein the at least one chromatin remodeler is selected from a polypeptide comprising HMGB1, HMGN1, HMGN2, HMGN3a, HMGN3b, HMGN4, HMGN5, histone H1.0, histone H1.2, CHD1, ISWI, DNA helicase, TOP1, herpesvirus 8 LANA, CMV IE1 or a domain thereof.

51. The system of any one of claims 1-50, wherein the at least one chromatin remodeler comprises an HMGB1 polypeptide, optionally wherein the HMGB1 polypeptide comprises a sequence at least 90%, 91%, 92%, 93%, 94%, 95%, 96%, 97%, 98%, 99%, or 100% identical to SEQ ID NO: 1783.

52. The system of claim 51, wherein the HMGB1 polypeptide comprises an HMGB1 Box B domain and at least 7 contiguous amino acid residues C-terminal to the HMGB1 Box B domain, and wherein the HMGB1 polypeptide lacks all or part of an acidic tail domain.

53. The system of any one of claims 51-52, wherein the HMGB1 polypeptide comprises a cryptic nuclear localization signal (NLS), optionally wherein the cryptic NLS comprises the sequence of EKSKKKK (SEQ ID NO: 1787).

54. The system of any one of claims 51-53, wherein the HMGB1 polypeptide lacks an HMGB1 Box A domain or wherein the HMGB1 polypeptide comprises two HMGB1 Box B domains, optionally wherein the cryptic NLS is located C-terminal to the C-terminal end of the two HMGB1 Box B domains.

55. The system of any one of claims 51-54, wherein the HMGB1 polypeptide comprises, from N-terminus to C-terminus:
1) HMGB1 Box A domain-HMGB1 Box B domain-cryptic NLS;
2) HMGB1 Box B domain-HMGB1 Box B domain-cryptic NLS; or
3) HMGB1 Box B domain-cryptic NLS.

56. The system of any one of claims 51-55, wherein the HMGB1 polypeptide comprises (i) at least 90% sequence identity to SEQ ID NO: 1783-1792 and 1803-1804; or (ii) the amino acid sequence of SEQ ID NO: 1783-1792 and 1803-1804.

57. The system of any one of claims 39-56, wherein the first or second polypeptide further comprises an HMGB1 polypeptide.

58. The system of claim 57, wherein the first or second polypeptide further comprises an HMGB1 polypeptide, further wherein: the first polypeptide comprises, from N-terminus to C-terminus: HMGB-nCas9-N-split-intein; and the second polypeptide comprises, from N-terminus to C-terminus: C-split-intein-DNA-dependent DNA polymerase.

59. The system of claim 57 or 58, wherein:
i) the first polypeptide comprises, from N-terminus to C-terminus: NLS-HMGB-linker-nCas9-linker-N-split-intein; and
ii) the second polypeptide comprises, from N-terminus to C-terminus:
1) C-split-intein-linker-NLS-DNA-dependent DNA polymerase-linker-coiled coil heterodimerization domain; or
2) C-split-intein-linker-NLS-DNA-dependent DNA polymerase-linker-NLS;
optionally wherein the second polypeptide comprises at least one enhancer of template-based genome editing at the C-terminus.

60. A template guide RNA (tgRNA), wherein the tgRNA comprises a first polynucleotide and a second polynucleotide, wherein:
i) the first polynucleotide comprises a SpyCas9 guide RNA, comprising, from 5′ to 3′,
1. a spacer that is complementary to a target region of a target strand of a DNA duplex target nucleic acid; and
2. a scaffold;
ii) the second polynucleotide is operably linked to the first polynucleotide and comprises, from 5′ to 3′:
1) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; and
2) a DNA-dependent DNA polymerase recruiting sequence (DRS) having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid, optionally wherein the DRS is 6-20 nucleotides in length, further optionally 6-17, 8-17, 10-12, 10-14, 10-16, or 12-14 nucleotides in length.

61. The tgRNA of claim 60, wherein the DRS comprises at least one of:
1) a sequence not fully complementary to the DRS-complementary region of the non-target strand of the DNA duplex target nucleic acid;
2) a 1-5 nucleotide, optionally a 1-3 nucleotide 3′ terminal tail comprising a 3′ terminal modified uridine nucleotide; or
3) a 5′ terminal (D1) DNA nucleotide and a penultimate 5′ terminal (D2) RNA nucleotide wherein the 3′ end of the first polynucleotide is covalently linked to the 5′ end of the second polynucleotide by a phosphorothioate or phosphodiester linkage.

62. The tgRNA of claim 60 or 61, wherein the spacer sequence is 20 nucleotides in length.

63. The tgRNA of any one of claims 60-62, wherein the spacer of the guide RNA comprises a modified nucleotide, optionally wherein the spacer of the guide RNA comprises a phosphorothioate (PS) modification, a 2′-O-methyl (2′-O-Me) modified nucleotide, or a 2′-F modified nucleotide.

64. The tgRNA of any one of claims 60-63, wherein the spacer of the guide RNA comprises DNA nucleotides, optionally wherein the spacer of the guide RNA comprises a DNA nucleotide at at least 2, 3, 4, 5, 6, 7, 8, 9, or 10 nucleotides, further optionally wherein the spacer of the guide RNA comprises a DNA nucleotide at 1, 2, 3, 4, 5, 6, 7, 8, 9, 10, or 11 nucleotides.

65. The tgRNA of any one of claims 60-64, wherein the spacer of the guide RNA comprises a DNA nucleotide at one or more, two or more, three or more, or four or more of positions S1, S2, S3, S4, S5, S9, S10, S11, S12, S13, S17, S18, or S20, optionally wherein the spacer of the guide RNA comprises a DNA nucleotide at one or more of positions S11, S12, and S18, optionally at each of positions S11, S12, and S18.

66. The tgRNA of any one of claims 60-63, wherein the spacer of the guide RNA does not comprise DNA nucleotides.

67. The tgRNA of any one of claims 60-64, wherein the spacer of the guide RNA comprises a modified nucleotide at one or more of the first five nucleotides at the 5′ end, optionally at one or more of the first three nucleotides at the 5′ end of the spacer.

68. The tgRNA of any one of claims 60-67, wherein the spacer of the guide RNA does not comprise a modification at one or more of positions S6, S7, S8, S15, S16, and S19, optionally at one or more of positions S4, S5, S6, S7, S8, S11, S12, S15, S16, S18, and S19.

69. The tgRNA of any one of claims 60-68, wherein the spacer of the guide RNA comprises a modification at one or more of positions S4, S5, S9, S10, S11, S12, S13, S14, S17, S18, and S20.

70. The tgRNA of any one of claims 60-69, wherein the spacer of the guide RNA comprises (i) 3 PS linkages between the 4 consecutive 5′ terminal nucleotides of the spacer; or (ii) a PS linkage between positions S7 and S8.

71. The tgRNA of any one of claims 60-70, wherein the spacer of the guide RNA comprises a 2′-O-Me modified nucleotide or a 2′-F modified nucleotide in at least 2, 3, 4, 5, 6, 7, 8, 9, or 10 nucleotides.

72. The tgRNA of any one of claims 60-71, wherein the spacer of the guide RNA comprises (i) 2′-O-Me modified nucleotides at each of the first three nucleotides (S1, S2, and S3) at the 5′ end of the spacer; or (ii) 2′-F modified nucleotides at one or more of positions S9, S10, S11, S13, S14, S17, and S18.

73. The tgRNA of claim 72, wherein the spacer of the guide RNA comprises a 2′-O-Me modified nucleotide at position S4 and 2′-F modified nucleotides at each of positions S9, S10, S11, S13, S14, S17, and S18, further optionally wherein the spacer of the guide RNA comprises a PS linkage between positions S7 and S8.

74. The tgRNA of any one of claims 60-72, wherein the spacer of the guide RNA comprises 2′-O-Me modified nucleotides at one or more of positions S4, S5, S9, S10, S11, S12, S13, S14, S17, S18, and S20, optionally wherein the spacer of the guide RNA comprises (i) 2′-O-Me modified nucleotides at one or more of positions S4, S5, S9, S10, S13, S14, S17 and S20; or (ii) 2′-O-Me modified nucleotides at each of positions S9, S10, S13, S14, S17, and S20.

75. The tgRNA of claim 74, wherein the spacer of the guide RNA comprises 2′-O-Me modified nucleotides at each of positions S9, S10, S17, and S20 and DNA nucleotides at each of positions S11, S12, and S18.

76. The tgRNA of any one of claims 60-72, wherein the spacer of the guide RNA comprises 2′-O-Me modified nucleotides at each of positions S1, S2, S3, S4, and 2′-F modified nucleotides at each of positions S9, S10, S11, S13, S14, S17, and S18, and unmodified nucleotides at each of positions S5, S6, S7, S8, S12, S15, S16, S19, an S20.

77. The system of any one of claims 60-72, wherein the spacer of the guide RNA comprises 2′-O-Me modified nucleotides at each of S1, S2, S3, S9, S10, S13, S14, S16, and S20, and unmodified nucleotides at each of S6, S7, S8, S15, S16, and S19, optionally unmodified nucleotides at one, two, three, four, or all of S4, S5, S11, S12, and S18.

78. The system of any one of claims 60-72, wherein the spacer of the guide RNA comprises 2′-O-Me modified nucleotides at each of S1, S2, S3, S9, S10, S13, S14, S17, and S20, DNA nucleotides at each of S11, S12, and S18, and unmodified nucleotides at each of S6, S7, S8, S15, S16, and S19, optionally unmodified nucleotides at one or both of S4 and S5.

79. The tgRNA of claim 74, wherein:
i) the spacer of the guide RNA comprises 2′-O-Me modified nucleotides at each of S1, S2, S3, S9, S10, S13, S14, S17, and S20; and
ii) the spacer of the guide RNA does not comprise a modification at each of S4, S5, S6, S7, S8, S11, S12, S15, S16, S18, and S19.

80. The tgRNA of claim 74, wherein:
i) the spacer of the guide RNA comprises 2′-O-Me modified nucleotides at each of S1, S2, S3, S9, S10, S13, S14, S17, and S20;
ii) the spacer of the guide RNA comprises DNA nucleotides at each of positions S11, S12, and S18; and
iii) the spacer of the guide RNA does not comprise a modification at each of S4, S5, S6, S7, S8, S15, S16, and S19.

81. The tgRNA of any one of claims 60-80, wherein the scaffold of the guide RNA comprises the nucleotide sequence of SEQ ID NO: 27 or 40.

82. The tgRNA of claim 81, wherein:
i) nucleotides 5′-GUUUUAGAGCUAGAAAUAGCAAGUUAAAAU-3′ or 5′-GUUUUAGACGUAGAAAUACGAAGUUAAAAU-3′ constitute a repeat/antirepeat (R/AR) region, wherein nucleotides 1-6 (LS1-LS6) and 25-30 (LS7-LS12) of the R/AR constitute a lower stem (LS) region, nucleotides 7-8 (B1-B2) and 21-24 (B3-B6) of the R/AR constitute a bulge region; and nucleotides 9-20 (US1-US12) of the R/AR constitute an upper stem (US) region;
ii) nucleotides 5′-AAGGCUAGUCCGUUAUCA-3′ constitute a nexus region (N1-N18);
iii) nucleotides 5′-CGAAAG-3′ constitute a hairpin 1 (H1) region (from 5′ to 3′, H1-2, H1-5 to H1-8, and H1-11);
iv) nucleotides 5′-GCACCGAGUCGGUGC-3′ constitute a hairpin 2 (H2) region H2-1 to H2-15); and
v) a G nucleotide between H1 region and H2 region constitutes nucleotide n;
wherein the scaffold comprises at least one region with unmodified and modified nucleotides selected from:
1) the lower stem region comprising modified nucleotides at LS1, LS8, LS10, and LS12; nucleotides not modified with 2′-O-Me at LS2, LS3, LS4, LS5;
2) the bulge region comprising nucleotides not modified with 2′-O-Me at B5 and B6;
3) the upper stem region comprising modified nucleotides at at least 10 positions selected from US1, US2, US3, US4, US5, US6, US7, US8, US9, US10, US11, and US12, optionally at all positions US1, US2, US3, US4, US5, US6, US7, US8, US9, US10, US11, and US12;
4) the nexus region comprising modified nucleotides at N1, N2, N4, N7, N11, N12, and N17; and nucleotides not modified with 2′-O-Me at N6, N8, N9, N10, N13, and N14;
5) the hairpin 1 (H1) region, comprising modified nucleotides at H1-2, H1-6, H1-7, H1-8, and H1-11; and nucleotides not modified with 2′-O-Me at H1-5;
6) the hairpin 2 (H2) region comprising modified nucleotides at H2-1, H2-2, H2-3, H2-4, H2-5, H2-6, H2-7, H2-12, H2-13, H2-14, and H2-15; and nucleotides not modified with 2′-O-Me at H2-8, H2-9, and H2-10; and
7) the n is a modified or unmodified nucleotide.

83. The tgRNA of claim 82, wherein the scaffold comprises at least one region with unmodified and modified nucleotides selected from:
i) the lower stem region comprising modified nucleotides at LS1, LS8, LS10, and LS12; nucleotides not modified with 2′-O-Me at LS2, LS3, LS4, LS5, and LS6;
ii) the bulge region comprising nucleotides not modified with 2′-O-Me at B5 and B6;
iii) the upper stem region comprising modified nucleotides at at least 10 positions selected from US1, US2, US3, US4, US5, US6, US7, US8, US9, US10, US11, and US12, optionally at all positions US1, US2, US3, US4, US5, US6, US7, US8, US9, US10, US11, and US12;
iv) the nexus region comprising modified nucleotides at N1, N2, N4, N7, N11, N12, and N17; and nucleotides not modified with 2′-O-Me at N6, N8, N9, N10, N13, N14, and N16;
v) the hairpin 1 (H1) region, comprising modified nucleotides at H1-2, H1-6, H1-7, H1-8, and H1-11; and nucleotides not modified with 2′-O-Me at H1-5;
vi) the hairpin 2 (H2) region comprising modified nucleotides at H2-1, H2-2, H2-3, H2-4, H2-5, H2-6, H2-7, H2-11, H2-12, H2-13, H2-14, and H2-15; and nucleotides not modified with 2′-O-Me at H2-8, H2-9, and H2-10; and
vii) the n is a modified or unmodified nucleotide.

84. The tgRNA of claim 81 or 82, wherein the scaffold comprises at least one region with unmodified and modified nucleotides selected from:
i) the lower stem region comprising modified nucleotides at LS1, LS8, LS10, and LS12; nucleotides not modified with 2′-O-Me at LS2, LS3, LS4, LS5, LS6, and LS9;
ii) the bulge region comprising nucleotides not modified with 2′-O-Me at B5 and B6;
iii) the upper stem region comprising modified nucleotides at at least 10 positions selected from US1, US2, US3, US4, US5, US6, US7, US8, US9, US10, US11, and US12, optionally at all positions US1, US2, US3, US4, US5, US6, US7, US8, US9, US10, US11, and US12;
iv) the nexus region comprising modified nucleotides at N1, N2, N4, N7, N11, N12, N16, and N17; and nucleotides not modified with 2′-O-Me at N6, N8, N9, N10, N13, N14, and N16;
v) the hairpin 1 (H1) region, comprising modified nucleotides at H1-2, H1-6, H1-7, H1-8, and H1-11; and nucleotides not modified with 2′-O-Me at H1-5;
vi) the hairpin 2 (H2) region comprising modified nucleotides at H2-1, H2-2, H2-3, H2-4, H2-5, H2-6, H2-7, H2-11, H2-12, H2-13, H2-14, and H2-15; and nucleotides not modified with 2′-O-Me at H2-8, H2-9, and H2-10; and
vii) the n is a modified or unmodified nucleotide.

85. The tgRNA of any one of claims 81-84, wherein the lower stem region comprises modified nucleotides at LS1, LS8, LS10, and LS12; nucleotides not modified with 2′-O-Me at LS2, LS3, LS4, LS5, LS6, LS7, LS9, and LS11.

86. The tgRNA of any one of claims 81-85, wherein the lower stem region comprises unmodified nucleotides at LS2, LS3, LS4, and LS5, optionally at LS2, LS3, LS4, LS5, LS6, LS7, LS9, and LS11, optionally wherein the lower stem region comprises unmodified nucleotides at LS7, LS9, and LS11.

87. The tgRNA of any one of claims 81-86, wherein the bulge region comprises (i) modified nucleotides at B2 and B3; or (ii) unmodified nucleotides at B5 and B6, optionally at B1, B4, B5, and B6.

88. The tgRNA of any one of claims 81-87, wherein the modified nucleotides of the upper stem are 2′-O-Me modified nucleotides.

89. The tgRNA of any one of claims 81-88, wherein the nexus region comprises unmodified nucleotides at N6, N8, N9, N10, N13, N14, and N18, optionally at N3, N5, N6, N8, N9, N10, N13, N14, N15, and N18.

90. The tgRNA of any one of claims 81-89, wherein the hairpin 1 region comprises (i) an unmodified nucleotide at H1-5; or (ii) unmodified nucleotides at H2-8, H2-9, and H2-10.

91. The tgRNA of any one of claims 81-90, further comprising a 3′ tail, optionally wherein the gRNA comprises a 3′ end modification.

92. The tgRNA of any one of claims 60-91, wherein the scaffold sequence comprises an internal linker, optionally wherein the scaffold sequence comprises (i) an internal linker in or substitutes for the upper stem region; (ii) an internal linker in a nexus region; or (iii) an internal linker in the hairpin 1 region.

93. The tgRNA of any one of claims 60-92, wherein the scaffold of the SpyCas9 guide RNA comprises a nucleotide sequence that is at least 90%, 91%, 92%, 93%, 93%, 94%, 95%, 96%, 97%, 98%, 99%, or 100% of any one of SEQ ID NOs: 5, 27, 35, 210, 212, 223, 269, 370, 201-208, 373-374, 393-400, 429, and 430; optionally,
i) the sequence of mGUUUUAGmAmGmC(L1)mGmCmAAGUUmAAmAAmUmAmAGmGCUmAGUCmC mGUUAUmCAmC(L1)mGGmGmCmAmCmCmGmAGUCmGmGmUmGmC (SEQ ID NO: 272) or a sequence having at least 90%, 95%, or 98% identity to SEQ ID NO: 272;
b) the sequence of GUUUUAGAmGmCmUmA(L1)mUmAmGmCAAGUUAAAAUAAGGCUAGUCCGUUA UCAC(L1)GGGCACCGAGUCGGmUmGmC (SEQ ID NO: 211) or a sequence having at least 90%, 95%, or 98% identity to SEQ ID NO: 211;
c) the sequence of GUUUUAGAmGmC(L1)mGmCAAGUUAAAAUAAGGCUAGUCCGUUAUCAC(L1) GG GCACCGAGUCGGmUmGmC (SEQ ID NO: 232) or a sequence having at least 90%, 95%, or 98% identity to SEQ ID NO: 232; or
d) the sequence of mGUUUUAGmAmGmCmUmA(L1)mUmAmGmCmAAGUUmAAmAAmUmAmAGmGC UmAGUCmCmGUUAUmCAmC(L1)mGGmGmCmAmCmCmGmAGUCmGmGmUmGmC (SEQ ID NO: 224) or a sequence having at least 90%, 95%, or 98% identity to SEQ ID NO: 224,
wherein L1 denotes that the linker is S18, and L3 denotes that the linker is S6.

94. The tgRNA of any one of claims 60-93, wherein the spacer or the scaffold of the guide RNA comprises a modified nucleotide.

95. The tgRNA of any one of claims 60-94, wherein the first polynucleotide comprises (i) a SpyCas9 guide RNA comprising, from 5′ to 3′, a guide sequence and any one of SEQ ID NOs: 5, 27, 35, 210, 212, 223, 269, 370, 201-208, 373-374, and 393-400; (ii) a SpyCas9 guide RNA comprising, from 5′ to 3′, a guide sequence and a nucleotide sequence that is at least 90%, 91%, 92%, 93%, 93%, 94%, 95%, 96%, 97%, 98%, 99%, or 100% of any one of SEQ ID NOs: 5, 27, 35, 210, 212, 223, 269, 370, 201-208, 373-374, 393-400, 429, and 430; or (iii) a SpyCas9 guide RNA comprising a nucleotide sequence that is at least 90%, 91%, 92%, 93%, 93%, 94%, 95%, 96%, 97%, 98%, 99%, or 100% of any one of SEQ ID NOs: 310, 312, 319-326, 347-349, 371, 384-392, 434, 438.

96. The tgRNA of any one of claims 60-95, wherein the first polynucleotide is covalently linked to the second polynucleotide, optionally wherein the 3′ end of the first polynucleotide is covalently linked to the 5′ end of the second polynucleotide.

97. The tgRNA of any one of claims 60-96, wherein the first polynucleotide is linked to the second polynucleotide by a non-nucleotide linker, optionally by a phosphodiester bond or a phosphorothioate bond.

98. The tgRNA of any one of claims 60-97, wherein the template comprises a mismatch to the genomic sequence, optionally 1-4 mismatches to the genomic sequence.

99. The tgRNA of any one of claims 60-98, wherein the mismatch that directs a template-based edit in the genomic DNA is within nucleotides T1-T25, optionally within nucleotides T1-T15 of the template.

100. The tgRNA of any one of claims 60-99, wherein the template comprises DNA nucleotides.

101. The tgRNA of any one of claims 60-100, wherein: (i) the 3′ terminal nucleotide of the template is a DNA nucleotide; or (ii) the template consists of DNA nucleotides from nucleotide 4 (T4) from the 3′ end of the template to the 5′ end of the template.

102. The tgRNA of any one of claims 60-101, wherein the template consists of DNA nucleotides.

103. The tgRNA of any one of claims 60-101, wherein the template comprises RNA nucleotides.

104. The tgRNA of any one of claims 60-101 and 103, wherein the template comprises both RNA nucleotide(s) and DNA nucleotide(s), optionally wherein at least one nucleotide at positions T1 or T2 of the template is an RNA nucleotide.

105. The tgRNA of any one of claims 60-101 and 103-104, wherein the template comprises 0-1 RNA nucleotides, optionally 1 RNA nucleotide, at the 3′ end of the template or wherein the template comprises 1-3 RNA nucleotides, optionally only 1 RNA nucleotide, at the 3′ end of the template.

106. The tgRNA of any one of claims 60-105, wherein (i) the DRS is 6-18 nucleotides in length, optionally 8-18 or 10-12 nucleotides in length; or (ii) the DRS is 10-16 or 12-14 nucleotides in length.

107. The tgRNA of any one of claims 60-106, wherein the DRS comprises (i) a sequence that is complementary to at least 6 nucleotides of the DRS-complementary region of the non-target strand of the DNA duplex target nucleic acid comprising nucleotides D2-D9 of the DRS, optionally D2-D8 or D3-D9 of the DRS; or (ii) a sequence that is complementary to a DRS-complementary region of a non-target strand of the DNA duplex target nucleic acid comprising nucleotides D6-D13 of the DRS, optionally wherein 1, 2, 3, 4, 5, 6, 7, or 8 of nucleotides D6-D13 are DNA nucleotides.

108. The tgRNA of any one of claims 60-107, wherein the DRS comprises a sequence that is not fully complementary to the DRS-complementary region of the non-target strand of the DNA duplex target nuclei acid.

109. The tgRNA of any one of claims 60-108, wherein the DRS comprises RNA nucleotides.

110. The tgRNA of any one of claims 60-109, wherein (i) the penultimate 5′ terminal nucleotide of the DRS (D2) is an RNA nucleotide; (ii) each nucleotide from the penultimate 5′ nucleotide (D2) to the 3′ terminal nucleotide in the DRS is an RNA nucleotide; or (iii) each nucleotide from the penultimate 5′ nucleotide (D2) to the 3′ end nucleotide in the DRS is an RNA nucleotide.

111. The tgRNA of any one of claims 60-110, wherein the DRS consists of RNA nucleotides.

112. The tgRNA of any one of claims 60-110, wherein the DRS comprises DNA nucleotides or wherein the DRS comprises both DNA nucleotide(s) and RNA nucleotide(s).

113. The tgRNA of any one of claims 60-110 and 112, wherein the DRS comprises a 3′ end and a 5′ end, and (i) the 5′ terminal nucleotide of the DRS (D1) is a DNA nucleotide; (ii) 0-7 nucleotides at the 5′ end of the DRS (nucleotides D1-D7) are DNA nucleotides; (iii) one or more of nucleotides 1-7 nucleotides at the 5′ end of the DRS (nucleotides D1-D7) are DNA nucleotides; or (iv) one or more of nucleotides 1-3 at the 5′ end of the DRS (nucleotides D1-D3), optionally the 5′ terminal nucleotide in the DRS (nucleotide D1) is a DNA nucleotide.

114. The tgRNA of claim 113, wherein:
i) one or more of nucleotides 1-5 at the 5′ end of the DRS (nucleotides D1-D5) are DNA nucleotides, optionally wherein the remaining nucleotides in the DRS are RNA nucleotides;
ii) 1-3 nucleotides at the 5′ end of the DRS (nucleotides D1-D3), optionally the most 5′ nucleotide in the DRS (nucleotide D1), is a DNA nucleotide;
iii) nucleotides 1 and 3 at the 5′ end of the DRS (nucleotides D1 and D3) are DNA nucleotides, optionally wherein the remaining nucleotides in the DRS are RNA nucleotides;
iv) nucleotides 1, 3, and 5 at the 5′ end of the DRS (nucleotides D1, D3, and D5) are DNA nucleotides, optionally wherein the remaining nucleotides in the DRS are RNA nucleotides; or
v) the 3′ terminal nucleotide in the DRS is an RNA nucleotide and the remaining nucleotides in the DRS are DNA nucleotides.

115. The tgRNA of any one of claims 60-110 and 112-114, wherein the 3′ terminal nucleotide of the template and the 5′ terminal nucleotide of the DRS are DNA nucleotides, and the penultimate 5′ terminal nucleotide of the DRS is an RNA nucleotide.

116. The tgRNA of any one of claims 60-110 and 112-113, wherein the 3′ terminal nucleotide in the DRS is an RNA nucleotide and the remaining nucleotides in the DRS are DNA nucleotides or wherein the DRS consists of DNA nucleotides.

117. The tgRNA of claim 116, wherein the spacer of the guide RNA comprises 2′-O-Me modified nucleotides at one or more of positions S1, S2, S3, S4, S5, S9, S10, S11, S12, S13, S14, S17, S18, and S20.

118. The tgRNA of claim 116, wherein the spacer of the guide RNA does not comprise a modification at one or more of positions S6, S7, S8, S15, S16, and S19.

119. The tgRNA of any one of claims 116-118, wherein:
i) the spacer of the guide RNA comprises 2′-O-Me modified nucleotides at each of S1, S2, S3, S9, S10, S13, S14, S17, and S20; and
ii) the spacer of the guide RNA does not comprise a modification at each of S4, S5, S6, S7, S8, S11, S12, S15, S16, S18, and S19.

120. The tgRNA of claim 114, wherein:
i) the spacer of the guide RNA comprises 2′-O-Me modified nucleotides at each of S1, S2, S3, S9, S10, S13, S14, S17, and S20;
ii) the spacer of the guide RNA comprises DNA nucleotides at each of positions S11, S12, and S18; and
iii) the spacer of the guide RNA does not comprise a modification at each of S4, S5, S6, S7, S8, S15, S16, and S19.

121. The tgRNA of any one of claims 60-120, wherein the DRS comprises a 3′ tail at the 3′ end of the DRS, wherein the tail is 1-5 nucleotides, optionally 1-3 nucleotides in length.

122. The tgRNA of claim 121, wherein the 3′ tail of the DRS comprises an RNA nucleotide.

123. The tgRNA of claim 121 or 122, wherein the 3′ tail comprises a uridine nucleotide, optionally wherein the 3′ terminal nucleotide is a modified uridine nucleotide.

124. The tgRNA of any one of claims 60-123, wherein the DRS comprises a modified nucleotide, optionally wherein the modified nucleotide is selected from:
i) 2′-O-methyl (2′-O-Me) modified nucleotide;
ii) 2′-fluoro (2′-F) modified nucleotide;
iii) an amine modified nucleotide;
iv) an alkoxy modified nucleotide;
v) an LNA modified nucleotide;
vi) methoxyethyl modified nucleotide;
vii) a halo modified nucleotide; or
viii) phosphorothioate (PS) linkage modified nucleotide;
optionally, wherein the modified nucleotide is a 2′-O-methyl (2′-O-Me) modified nucleotide or a phosphorothioate (PS) linkage modified nucleotide.

125. The tgRNA of claim 124, wherein
i) the modification in the DRS comprises a phosphorothioate modification, and the DRS does not comprise a phosphorothioate modification at position D1-D9 in the DRS sequence;
ii) the DRS comprises a phosphorothioate modification at or 3′ of position D10 in the DRS sequence;
iii) the modification in the DRS comprises a 2′-O-Me modification, and the DRS does not comprise a 2′-O-Me modification at position D1-D3 in the DRS sequence;
iv) the modification in the DRS comprises a 2′-O-Me modification, and the DRS does not comprise a 2′-O-Me modification at the 5′ terminal and 3′ terminal nucleotides of the DRS;
v) the modification in the DRS comprises a 2′-O-Me modification at a nucleotide other than the 5′ terminal and 3′ terminal nucleotides of the DRS;
vi) the modification in the DRS comprises a 2′-O-Me modification, and the DRS comprises a 2′-O-Me modification at or 3′ of position D4 in the DRS sequence;
vii) the modification in the DRS comprises a 2′-O-Me or a 2′-F modification, and the DRS comprises a 2′-O-Me or 2′-F modification at the 3′ terminal nucleotide in the DRS;
viii) 1-3 terminal nucleotides at the 3′ end of the DRS are chemically modified, optionally wherein the nucleotide at the 3′ end of the DRS is chemically modified;
ix) 1-3 terminal nucleotides at the 3′ end of the DRS comprises a phosphorothioate (PS) modification, optionally wherein at least one PS modification is between the last two nucleotides at the 3′ terminus;
x) the 3′ terminal nucleotide of the DRS comprises a 2′-O-Me modification;
xi) the DRS comprises PS linkages between 2, 3, 4, 5, or 6 consecutive 3′ terminal nucleotides of the DRS; or
xii) all RNA nucleotides in the DRS comprise modified RNA nucleotides.

126. The tgRNA of any one of claims 124-125, wherein:
i) the DRS comprises PS linkages between 2, 3, 4, 5, or 6 consecutive 3′ terminal nucleotides of the DRS;
ii) the DRS comprises a PS linkage between the 3′ terminal and penultimate 3′ terminal nucleotides of the DRS;
iii) the DRS comprises 3 PS linkages between the 4 consecutive 3′ terminal nucleotides of the DRS;
iv) the DRS comprises a PS linkage between 2 nucleotides other than the 3′ terminal and penultimate 3′ terminal nucleotides of the DRS;
v) the DRS comprises a phosphorothioate modification, wherein the DRS does not comprise a phosphorothioate modification at position D1-D9 in the DRS sequence; or
vi) the DRS comprises a phosphorothioate modification at or 3′ of position D10 in the DRS sequence.

127. The tgRNA of any one of claims 60-95 and 98-126, wherein the first polynucleotide is not covalently linked to the second polynucleotide.

128. The tgRNA of any one of claims 60-110, and 112-127, wherein
i) 1-7 consecutive 5′ terminal nucleotides of the DRS are DNA nucleotides;
ii) the 5′ terminal and penultimate 5′ terminal nucleotides of the DRS are DNA nucleotides;
iii) at least 3 consecutive 5′ terminal nucleotides of the DRS are DNA nucleotides; or
iv) 3 consecutive 5′ terminal nucleotides of the DRS are DNA nucleotides.

129. The tgRNA of any one of claims 60-128, wherein the tgRNA further comprises an affinity tag, wherein the first polynucleotide or the second polynucleotide are bound to a peptide or a peptide complex comprising an nCas9 and a DNA-dependent DNA polymerase by the affinity tag.

130. The tgRNA of any one of claims 60-110, and 112-129, wherein 1-7 consecutive 5′ terminal nucleotides of the DRS are DNA nucleotides, optionally wherein:
i) the 5′ terminal and penultimate 5′ terminal nucleotides of the DRS are DNA nucleotides;
ii) at least 3 consecutive 5′ terminal nucleotides of the DRS are DNA nucleotides; or
iii) 3 consecutive 5′ terminal nucleotides of the DRS are DNA nucleotides.

131. The tgRNA of any one of claims 129-130, wherein the affinity tag is located 5′ of the template sequence or the affinity tag is located 3′ of the DRS.

132. The tgRNA of any one of claims 129-131, wherein the tgRNA comprises two affinity tags, optionally wherein (i) both affinity tags are located 5′ of the template; or (ii) a first affinity tag is located 5′ of the template sequence and a second affinity tag is located 3′ of the DRS, wherein the DNA-dependent DNA polymerase is T5 DNA polymerase.

133. The tgRNA of any one of claims 129-132, wherein the affinity tag is an aptamer comprising one or more modified nucleotides, optionally wherein the aptamer is located 5′ of the template and has a 5′ terminus, further optionally wherein the aptamer comprises one or more modified nucleotide at the 5′ end thereof.

134. The tgRNA of claim 133, wherein the modified nucleotide is a 2′-O-methyl (2′-O-Me) modified nucleotide or a phosphorothioate (PS) linkage modified nucleotide.

135. The tgRNA of any one of claims 133-134, wherein the aptamer comprises a stem-loop, optionally wherein the aptamer comprises 2′-O-Me modified nucleotides at each nucleotide in the stem of the stem-loop.

136. The tgRNA of any one of claims 133-135, wherein the aptamer is located 5′ of the template sequence, further wherein:
i) the aptamer comprises a PS linkage between each of the first three nucleotides at the 5′ end;
ii) the aptamer is located 5′ of the template sequence, further wherein the aptamer comprises 2′-O-Me modified nucleotides at each of the first two nucleotides at the 5′ end;
iii) the aptamer comprises 2′-O-Me modified nucleotides at each of the first two nucleotides at the 5′ end and a PS linkage between each of the first three nucleotides at the 5′ end; or
iv) the aptamer comprises 2′-O-Me modified nucleotides at each nucleotide in the stem of the stem-loop and a PS linkage between each of the first three nucleotides at the 5′ end.

137. The tgRNA of any one of claims 133-136, wherein the aptamer is located 3′ of the DRS, further wherein: (i) the affinity tag comprises a PS linkage between the last two nucleotides at the 3′ end; or (ii) the aptamer comprises 2′-O-Me modified nucleotides at each nucleotide in the stem of the stem-loop and a PS linkage between the last two nucleotides at the 3′ end.

138. The tgRNA of any one of claims 133-137, wherein the aptamer comprises 2′-O-Me modified nucleotides at each nucleotide.

139. A system comprising:
a) a first polynucleotide encoding a first polypeptide comprising a DNA-dependent DNA polymerase, or portion thereof, operably linked to a first split-intein; and
b) a second polynucleotide encoding a second polypeptide comprising a SpyCas9 nickase (nCas9), or portion thereof, operably linked to a second split-intein that is complementary to the first split-intein, wherein the first split-intein and the second split-intein undergo trans-splicing reaction, resulting in the formation of a single fusion polypeptide comprising from N-terminus to C-terminus, (i) the DNA-dependent DNA polymerase and the nCas9, or (ii) the nCas9 and the DNA-dependent DNA polymerase.

140. The system of claim 139, comprising:
i) a first polynucleotide encoding the DNA-dependent DNA polymerase operably linked to the first split-intein; and (ii) a second polynucleotide encoding the nCas9 operably linked to the second split-intein that is complementary to the first split-intein;
ii) a first polynucleotide encoding a first portion of the DNA-dependent DNA polymerase operably linked to the first split-intein; and (ii) a second polynucleotide encoding a second portion of the DNA-dependent DNA polymerase and the nCas9 operably linked to the second split-intein that is complementary to the first split-intein; or
iii) a first polynucleotide encoding the DNA-dependent DNA polymerase and a first portion of the nCas9 operably linked to the first split-intein; and (ii) a second polynucleotide encoding a second portion of the nCas9 operably linked to the second split-intein that is complementary to the first split-intein.

141. The system of claim 139 or 140, wherein:
i) the DNA-dependent DNA polymerase is a polK comprising an amino acid sequence that has at least 90%, 91%, 92%, 93%, 94%, 95%, 96%, 97%, 98%, 99%, or 100% identity to amino acid residues 19-526 of SEQ ID NO: 1021; or an amino acid sequence that has at least 90%, 91%, 92%, 93%, 94%, 95%, 96%, 97%, 98%, 99%, or 100% identity to SEQ ID NO: 1021, 1265, or 1206, optionally wherein the polk includes or lacks an N-terminal methionine relative to SEQ ID NO: 1021, 1265, or 1206; or
ii) the DNA-dependent DNA polymerase is a T5 pol comprising an amino acid sequence that has at least 90%, 91%, 92%, 93%, 94%, 95%, 96%, 97%, 98%, 99%, or 100% identity to any one of SEQ ID NO: 1116 or 1121, optionally wherein the T5 pol includes or lacks an N-terminal methionine relative to SEQ ID NO: 1116 or 1121.

142. The system of any one of claims 139-141, wherein the first split-intein is an N-split-intein and the second split-intein is a C-split-intein or wherein the first split-intein is a C-split-intein and the second split-intein is an N-split-intein, optionally wherein the first split-intein and second split-intein are Cfa-inteins.

143. The system of any one of claims 139-142, wherein:
1) the first polynucleotide comprises an open reading frame (ORF) of at least 90%, 91%, 92%, 93%, 94%, 95%, 96%, 97%, 98%, 99%, or 100% identical to any one of SEQ ID NOs: 1278, 1281, 1363, 1366, and 1378; 1391, 1395, 1399, 1403, 1407, 1411, 1415, 1419, 1423, 1427, 1431, 1435, 1439, 1443, 1907, 1913, 1940, 1943 or a sequence at least 90%, 91%, 92%, 93%, 94%, 95%, 96%, 97%, 98%, 99%, or 100% identical to any one of SEQ ID NOs: 1277, 1280, 1362, 1365, 1377, 1906, 1912, 1939, 1942, 3006-3008, 3010, 3012, and 3017-3018; or
2) the second polynucleotide comprises an open reading frame (ORF) of at least 90%, 91%, 92%, 93%, 94%, 95%, 96%, 97%, 98%, 99%, or 100% identical to any one of SEQ ID NOs: 1284, 1287, 1290, 1293, 1296, 1369, 1381, 1384, 1389, 1393; 1397, 1401, 1405, 1409, 1413, 1417, 1421, 1425, 1429, 1433, 1437, 1441, 1901, 1904, 1910, 1916, 1918, 1946, 1949, 1952, 1955, 1958 or a sequence at least 90%, 91%, 92%, 93%, 94%, 95%, 96%, 97%, 98%, 99%, or 100% identical to any one of SEQ ID NOs: 1283, 1286, 1289, 1292, 1295, 1368, 1380, 1383, 1900, 1903, 1909, 1915, 1919, 1945, 1948, 1951, 1954, 1957, 3001-3005, 3009, 3011, 3013-3016, and 3019-3022.

144. A system comprising: (a) a DNA-dependent DNA polymerase and a SpyCas9 nickase (nCas9); and (b) at least one chromatin remodeler operably linked to the nCas9 and the DNA-dependent DNA polymerase.

145. The system of claim 144, further comprising a nuclear localization signal, wherein the DNA-dependent DNA polymerase and the nCas9 are covalently linked sequentially in a polypeptide from N-terminus to C-terminus, wherein the order is:
i) nuclear localization signal-chromatin remodeler-nCas9-DNA Dependent DNA polymerase-nuclear localization signal;
ii) nuclear localization signal-nCas9-chromatin remodeler-DNA Dependent DNA polymerase-nuclear localization signal; or
iii) nuclear localization signal-nCas9-DNA Dependent DNA polymerase-chromatin remodeler-nuclear localization signal.

146. The system of any one of claims 144-145, wherein the at least one chromatin remodeler comprises an HMGB1 polypeptide.

147. The system of claim 146, wherein the HMGB1 polypeptide comprises an HMGB1 polypeptide, wherein the HMGB1 polypeptide comprises an HMGB1 Box B domain and at least 7 contiguous amino acid residues C-terminal to the HMGB1 Box B domain, and wherein the HMGB1 polypeptide lacks all or part of an acidic tail domain.

148. The system of claim 146 or 147, wherein the HMGB1 polypeptide comprises a cryptic nuclear localization signal (NLS), optionally wherein the cryptic NLS comprises the sequence of EKSKKKK (SEQ ID NO: 1787).

149. The system of any one of claims 146-148, wherein the HMGB1 polypeptide lacks an HMGB1 Box A domain.

150. The system of any one of claims 146-149, wherein the HMGB1 polypeptide comprises two HMGB1 Box B domains, optionally wherein the cryptic NLS is located C-terminal to the C-terminal end of the two HMGB1 Box B domains.

151. The system of any one of claims 146-150, wherein the HMGB1 polypeptide comprises at least 90% sequence identity to SEQ ID NO: 1783-1792 or 1803-1804, or wherein the HMGB1 polypeptide comprises the amino acid sequence of SEQ ID NO: 1783-1792 or 1803-1804.

152. The system of any one of claims 139-151, further comprising a template guide RNA (tgRNA), wherein the tgRNA comprises a first polynucleotide and a second polynucleotide, wherein:
a) the first polynucleotide of the tgRNA comprises a SpyCas9 guide RNA comprising, from 5′ to 3′:
i) a spacer that is complementary to a target region of a target strand of a DNA duplex target nucleic acid; and
ii) a scaffold; and
b) the second polynucleotide of the tgRNA is operably linked to the first polynucleotide of the tgRNA and comprises, from 5′ to 3′:
i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides;
ii) a DNA-dependent DNA polymerase recruiting sequence (DRS) that is 6-18 nucleotides in length, optionally 8-18 or 10-12 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the nontarget strand of the DNA duplex target nucleic acid.

153. A system comprising a DNA polymerase kappa (polK), a SpyCas9 nickase (nCas9), and a template guide RNA (tgRNA), wherein the tgRNA comprises a first polynucleotide and a second polynucleotide, wherein:
a) the first polynucleotide comprises a SpyCas9 guide RNA comprising, from 5′ to 3′:
i) a spacer that is complementary to a target region of a target strand of a DNA duplex target nucleic acid; and
ii) a scaffold; and
b) the second polynucleotide is operably linked to the first polynucleotide and comprises, from 5′ to 3′:
i) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides;
ii) a DNA-dependent DNA polymerase recruiting sequence (DRS) that is 6-18 nucleotides in length, optionally 8-18 or 10-12 nucleotides in length and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid, wherein the DRS comprises RNA nucleotides.

154. The system of claim 153, wherein the polk comprises an amino acid sequence that has at least 90%, 91%, 92%, 93%, 94%, 95%, 96%, 97%, 98%, 99%, or 100% identity to amino acid residues 19-526 of SEQ ID NO: 1021; or an amino acid sequence that has at least 90%, 91%, 92%, 93%, 94%, 95%, 96%, 97%, 98%, 99%, or 100% identity to SEQ ID NO: 1021, optionally wherein the polk includes or lacks an N-terminal methionine relative to SEQ ID NO: 1021.

155. The system of claim 153 or 154, wherein the polK comprises amino acids 19-526, optionally amino acids 1-526 of polK relative to SEQ ID NO: 1021.

156. The system of any one of claims 335-337, wherein the polk comprises a mutation at G411 or E412 relative to SEQ ID NO: 1021, optionally wherein the polk comprises a mutation at G411 and E412.

157. The system of any one of claims 153-156, wherein the polk comprises GE411-412RV mutations relative to SEQ ID NO: 1021.

158. The system of any one of claims 153-157, wherein polK is encoded by:
a) a nucleotide sequence that has at least 90%, 91%, 92%, 93%, 94%, 95%, 96%, 97%, 98%, 99%, or 100% identity to a coding sequence for amino acid residues 19-526 within SEQ ID NO: 1021;
a) a nucleotide sequence that has at least 90%, 91%, 92%, 93%, 94%, 95%, 96%, 97%, 98%, 99%, or 100% identity to SEQ ID NO: 1020;
b) a nucleotide sequence that has at least 90%, 91%, 92%, 93%, 94%, 95%, 96%, 97%, 98%, 99%, or 100% identity to SEQ ID NO: 1201;
c) a nucleotide sequence that has at least 90%, 91%, 92%, 93%, 94%, 95%, 96%, 97%, 98%, 99%, or 100% identity to SEQ ID NO: 1203;
d) a nucleotide sequence that has at least 90%, 91%, 92%, 93%, 94%, 95%, 96%, 97%, 98%, 99%, or 100% identity to SEQ ID NO: 1205;
e) a nucleotide sequence that has at least 90%, 91%, 92%, 93%, 94%, 95%, 96%, 97%, 98%, 99%, or 100% identity to SEQ ID NO: 1264; or
f) a nucleotide sequence that has at least 90%, 91%, 92%, 93%, 94%, 95%, 96%, 97%, 98%, 99%, or 100% identity to SEQ ID NO: 1018.

159. The system of any one of claims 153-158, wherein the polk or the nCas9 is linked to a heterologous nuclear localization signal (NLS).

160. The system of any one of claims 153-159, wherein the polK is linked to an MS2 coat protein (MCP) domain.

161. The system of any one of claims 153-160, wherein the polk is covalently linked to the nCas9.

162. The system of any one of claims 153-161 wherein the polk and nCas9 are covalently linked sequentially in a polypeptide from N-terminus to C-terminus in an order selected from:
1) NLS-nCas9-NLS-polK;
2) NLS-nCas9-linker-NLS-linker-polK;
3) polK-NLS-nCas9-NLS;
4) polK-linker-NLS-linker-nCas9-linker-NLS;
5) NLS-nCas9-NLS-polK (GE411-412RV);
6) NLS-nCas9-linker-NLS-polK (GE411-412RV);
7) NLS-nCas9-NLS-polK (19-526);
8) NLS-nCas9-linker-NLS-linker-polK (19-526);
9) polK (19-526)-NLS-nCas9-NLS;
10) polK (19-526)-Linker-NLS-linker-nCas9-linker-NLS;
11) polK (GE411-412RV)-NLS-NLS-nCas9;
12) polK (GE411-412RV)-linker-NLS-linker-NLS-nCas9;
13) NLS-nCas9-NLS-polK (19-526, GE411-412RV);
14) NLS-nCas9-linker-NLS-linker-polK (19-526, GE411-412RV);
15) NLS-nCas9-NLS-polK-NLS;
16) NLS-nCas9-linker-NLS-linker-polK-linker-NLS;
17) NLS-nCas9-NLS-polK (1-526, GE411-412RV)-NLS;
18) NLS-nCas9-linker-NLS-linker-polK (1-526, GE411-412RV)-linker-NLS;
19) nCas9-polK (1-526);
20) polK (1-526, GE411-412RV)-nCas9-NLS-heterodimerization domain, optionally wherein the heterodimerization domain comprises a coiled coil heterodimerization domain, optionally an EI domain or a KI domain; or
21) polK (1-526, GE411-412RV)-linker-NLS-nCas9-linker-NLS-heterodimerization domain, optionally wherein the heterodimerization domain comprises a coiled coil heterodimerization domain, optionally an EI domain or a KI domain;
optionally wherein the order is NLS-nCas9-linker-NLS-linker-polK (1-526, GE411-412RV)-linker-NLS, optionally further comprising a heterodimerization domain comprising a coiled coil heterodimerization domain, optionally an EI domain or a KI domain.

163. The system of any one of claims 153-162, wherein the polK, or portion thereof, is operably linked to a first split-intein and the nCas9, or portion thereof, is operably linked to a second split-intein that is complementary to the first split intein, wherein the first split-intein and second split-intein undergo trans-splicing reaction, resulting in the formation of a single fusion polypeptide comprising, from N-terminus to C-terminus, (i) the polk and the nCas9 or (2) the polk and the nCas9.

164. The system of any one of claim 163, wherein the polK is operably linked to a heterologous nuclear localization signal (NLS) or, wherein the nCas9 is operably linked to a heterologous nuclear localization signal (NLS).

165. The system of any one of claims 153-164, wherein the polk and nCas9 are operably linked to at least one chromatin remodeler, and wherein the polk, nCas9, and chromatin remodeler are covalently linked sequentially in a polypeptide from N-terminus to C-terminus in an order selected from:
ii) chromatin remodeler-nCas9-polK;
iii) nCas9-chromatin remodeler-polK; or
iv) nCas9-polK-chromatin remodeler.
optionally further comprising a nuclear localization signal (NLS)

166. The system of any one of claim 165, wherein the at least one chromatin remodeler comprises an HMGB1 polypeptide.

167. The system of claim 166, wherein the HMGB1 polypeptide comprises (i) at least 90% sequence identity to SEQ ID NO: 1783-1792 and 1803-1804 or (ii) the amino acid sequence of SEQ ID NO: 1783-1792 and 1803-1804.

168. The system of any one of claims 153-160, wherein the polK and nCas9 are not covalently linked, and the polK is provided with a 3′ extension recruiting domain, wherein, the polk and the 3′ extension recruiting domain are covalently linked sequentially in a polypeptide from N-terminus to C-terminus in an order selected from:
1) 3′ extension recruiting domain-polK-NLS;
2) 3′ extension recruiting domain-linker-polK-linker-NLS;
3) 3′ extension recruiting domain-polK (GE411-412RV)-NLS; and
4) 3′ extension recruiting domain-linker-polK (GE411-412RV)-linker-NLS.

169. The system of any one of claim 168, wherein the polypeptide comprises a heterodimerization domain or further comprises an additional heterodimerization domain.

170. The system of any one of claims 161-169, wherein (i) the polypeptide comprises a sequence selected from any one of SEQ ID NOs: 1024, 1027, 1030, 1036, 1039, 1042, 1045, 1048, 1053, 1056, 1059, 1074, 1200, 1202, 1229, 1234, or 1268, or 1300, 1373, 1388, 1447, 1453, 1923, 1926, or 1929; or (ii) the polypeptide is encoded by a nucleotide sequence that has at least 90%, 91%, 92%, 93%, 94%, 95%, 96%, 97%, 98%, 99%, or 100% identity to any one of SEQ ID NOs: 1023, 1026, 1029, 1035, 1038, 1041, 1044, 1047, 1052, 1055, 1058, 1073, 1199, 1201, 1228, 1233, or 1267, and 1299, 1372, 1387, 1446, 1452, 1922, 1925, or 1928.

171. The system of any one of claims 161-170, wherein (i) the polypeptide comprises a sequence selected from any one of SEQ ID NOs: 1062 and 1067; or (ii) the polypeptide is encoded by a nucleotide sequence that has at least 90%, 91%, 92%, 93%, 94%, 95%, 96%, 97%, 98%, 99%, or 100% identity to any one of SEQ ID NOs: 1061 and 1066.

172. A system comprising a T5 DNA polymerase (T5 pol), a SpyCas9 nickase (nCas9), and a template guide RNA (tgRNA), wherein the tgRNA comprises a first polynucleotide and a second polynucleotide, wherein:
1) the first polynucleotide comprises a SpyCas9 guide RNA comprising, from 5′ to 3′:
a. a spacer that is complementary to a target region of a target strand of a DNA duplex target nucleic acid; and
b. a scaffold; and
2) the second polynucleotide is operably linked to the first polynucleotide and comprises, from 5′ to 3′:
a. a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides; and
b. a DNA-dependent DNA polymerase recruiting sequence (DRS) that is 6-20 nucleotides in length, optionally 10-16 or 12-14 nucleotides in length, and having a 3′ end and a 5′ end; wherein the 5′ end of the DRS is covalently attached to the 3′ end of the template sequence, and wherein the DRS comprises a sequence that is complementary to at least 6 nucleotides of a DRS-complementary region of the non-target strand of the DNA duplex target nucleic acid comprising nucleotides 5-10 5′ of the PAM in the non-target strand of the DNA duplex target nucleic acid, wherein the DRS comprises RNA nucleotides.

173. The system of claim 172, wherein the T5 pol comprises an amino acid sequence that has at least 90%, 91%, 92%, 93%, 94%, 95%, 96%, 97%, 98%, 99%, or 100% identity to any one of SEQ ID NO: 1116 or SEQ ID NO: 1121, optionally wherein the T5 pol includes or lacks an N-terminal methionine relative to SEQ ID NO: 1116 or 1121.

174. The system of claim 172 or 173, wherein the T5 pol comprises a mutation at D164 or E166 relative to SEQ ID NO: 1116.

175. The system of any one of claims 172-174, wherein the T5 pol comprises D164A and E166A mutations relative to SEQ ID NO: 1116.

176. The system of any one of claims 172-175, wherein the T5 pol comprises an amino acid sequence comprising an N-terminal deletion of 30 amino acids relative to SEQ ID NO: 1116.

177. The system of any one of claims 172-176, wherein the T5 pol is encoded by a nucleotide sequence that has at least 90%, 91%, 92%, 93%, 94%, 95%, 96%, 97%, 98%, 99%, or 100% identity to any one of SEQ ID NOs: 1115 or SEQ ID NO: 1120, optionally wherein the T5 pol includes or lacks a start or stop codon relative to SEQ ID NO: 1115 or 1120.

178. The system of any one of claims 172-177, wherein the template comprises 0-1 RNA nucleotides, optionally 1 RNA nucleotide, at the 3′ end of the template.

179. The system of any one of claims 172-178, wherein the T5 pol or the nCas9 is linked to a heterologous nuclear localization signal (NLS).

180. The system of any one of claims 172-179, wherein the T5 pol is linked to an MS2 coat protein (MCP domain).

181. The system of any one of claims 172-180, wherein the T5 pol is covalently linked to the nCas9.

182. The system of any one of claims 172-181, wherein the T5 pol and nCas9 are covalently linked sequentially in a polypeptide from N-terminus to C-terminus in an order selected from:
1) nCas9-T5Pol-NLS;
2) nCas9-linker-T5Pol-linker-NLS;
3) T5Pol-nCas9-NLS;
4) T5Pol-linker-nCas9-linker-NLS;
5) NLS-nCas9-NLS-T5Pol-binding site-NLS-NLS;
6) NLS-nCas9-linker-NLS-linker-T5Pol-linker-MCP-linker-NLS-linker-NLS;
7) nCas9-linker-T5Pol-linker-NLS;
8) nCas9-T5Pol(N30del)-NLS;
9) nCas9-linker-T5Pol(N30del)-linker-NLS;
10) nCas9-T5Pol(DA593R)-NLS;
11) nCas9-linker-T5Pol(A593R)-linker-NLS;
12) NLS-nCas9-NLS-T5Pol-NLS-NLS;
13) NLS-nCas9-linker-NLS-linker-T5Pol-linker-NLS-linker-NLS;
14) nCas9-NLS-T5Pol;
15) nCas9-linker-NLS-linker-T5Pol;
16) NLS-nCas9-NLS-T5Pol-NLS-NLS;
17) NLS-nCas9-linker-NLS-linker-T5Pol-linker-NLS-NLS;
18) nCas9-T5Pol(K425I)-NLS;
19) nCas9-linker-T5Pol(K425I)-linker-NLS;
20) nCas9-T5Pol-NLS-heterodimerization domain, optionally wherein the heterodimerization domain comprises a coiled coil heterodimerization domain, optionally an EI domain or a KI domain; or
21) nCas9-linker-T5Pol-linker-NLS-linker-heterodimerization domain, optionally wherein the heterodimerization domain comprises a coiled coil heterodimerization domain, optionally an EI domain or KI domain, and
optionally, wherein the T5pol comprises a mutation at D164 or E166 relative to SEQ ID NO: 1116, optionally wherein the T5 pol comprises D164A and E166A mutations relative to SEQ ID NO: 1116.

183. The system of any one of claims 172-182, wherein the T5 pol and nCas9 are covalently linked sequentially in a polypeptide from N-terminus to C-terminus in order wherein the order is NLS-nCas9-linker-NLS-linker-T5Pol-linker-NLS-NLS; optionally, wherein the T5pol comprises a mutation at D164 or E166 relative to SEQ ID NO: 1116, optionally wherein the T5 pol comprises D164A and E166A mutations relative to SEQ ID NO: 1116.

184. The system of any one of claims 172-183, wherein the T5Pol and nCas9 are covalently linked sequentially in a polypeptide from N-terminus to C-terminus, wherein the order is NLS-nCas9-linker-NLS-linker-T5Pol-linker-NLS-NLS-linker, wherein the T5Pol comprises a mutation at D164 or E166 relative to SEQ ID NO: 1116.

185. The system of any one of claims 172-179, wherein the T5 pol or a portion thereof, is operably linked to a first split-intein and the nCas9, or a portion thereof, is operably linked to a second split-intein that is complementary to the first split intein, wherein the first split-intein and second split-intein undergo trans-splicing reaction, resulting in the formation of a single fusion polypeptide comprising, from N-terminus to C-terminus, the polk and the nCas9.

186. The system of claim 185, wherein the T5 pol is operably linked to a heterologous nuclear localization signal (NLS) or wherein the nCas9 is operably linked to a heterologous nuclear localization signal (NLS).

187. The system of any one of claims 172-186, wherein the T5 pol and the nCas9 are covalently linked, and wherein the T5 pol and the nCas9 are operably linked to at least one chromatin remodeler, further wherein the T5 pol, nCas9, and chromatin remodeler are covalently linked sequentially in a polypeptide from N-terminus to C-terminus in an order selected from:
1) chromatin remodeler-nCas9-T5 pol;
2) nCas9-chromatin remodeler-T5 pol; or
3) nCas9-T5 pol-chromatin remodeler.

188. The system of claim 187, wherein the at least one chromatin remodeler comprises an HMGB1 polypeptide.

189. The system of claim 188, wherein the HMGB1 polypeptide comprises (i) at least 90% sequence identity to SEQ ID NO: 1783-1792 and 1803-1804 or (ii) the amino acid sequence of SEQ ID NO: 1783-1792 and 1803-1804.

190. The system of any one of claims 185-189, wherein the T5 pol and the nCas9 are not covalently linked, and the T5 pol is provided with a 3′ extension-recruiting domain, wherein, the T5 pol and the 3′ extension-recruiting domain are covalently linked sequentially in a polypeptide from N-terminus to C-terminus in an order selected from:
i) 3′ extension-recruiting domain-T5Pol(D164A,E166A)-NLS; or
ii) 3′ extension-recruiting domain-linker-T5Pol(D164A,E166A)-linker-NLS.

191. The system of claim 190, wherein each NLS is independently selected from an SV40 NLS, a nucleoplasmin NLS, or a c-Myc NLS.

192. The system of any one of claims 179-184 and 187-191, wherein (i) the polypeptide comprises a sequence that has at least 90%, 91%, 92%, 93%, 94%, 95%, 96%, 97%, 98%, 99%, or 100% identity to any one of SEQ ID NO: 1124, 1127, 1129, 1132, 1134, 1137, 1140, 1142, 1145, 1147, 1150, 1152, 1155, 1157, 1160, 1162, 1165, 1167, 1170, 1172, 1216, 1219, 1224, 1254, and 1259, 1276, 1303, 1307, 1376, 1450, 1932, 1935, and 1938; or (ii) the polypeptide comprises a sequence that has at least 90%, 91%, 92%, 93%, 94%, 95%, 96%, 97%, 98%, 99%, or 100% identity to any one of SEQ ID NO: 1124, 1129, 1134, 1137, 1142, 1147, 1152, 1157, 1162, 1167, 1172, 1219, 1224, 1254, and 1259, 1276, 1307, 1932, 1935, and 1938.

193. The system of any one of claims 179-184 and 187-192, wherein (i) the polypeptide comprises a sequence selected from any one of SEQ ID NO: 1124, 1127, 1129, 1132, 1134, 1137, 1140, 1142, 1145, 1147, 1150, 1152, 1155, 1157, 1160, 1162, 1165, 1167, 1170, 1172, 1216, 1219, 1224, 1254, and 1259, 1276, 1303, 1307, 1376, 1450, 1932, 1935, and 1938 or comprises a sequence selected from any one of SEQ ID NO: 1124, 1129, 1134, 1137, 1142, 1147, 1152, 1157, 1162, 1167, 1172, 1219, 1224, 1254, and 1259, 1276, 1307, 1932, 1935, and 1938; (ii) is encoded by a nucleotide sequence that has at least 90%, 91%, 92%, 93%, 94%, 95%, 96%, 97%, 98%, 99%, or 100% identity to any one of SEQ ID NOs: 1123, 1126, 1128, 1131, 1133, 1136, 1139, 1141, 1144, 1146, 1149, 1151, 1154, 1156, 1159, 1161, 1164, 1166, 1169, 1171, 1215, 1218, 1223, 1253, and 1258, 1275, 1302, 1306, 1375, 1449, 1931, 1934, and 1937; or (iii) is encoded by a nucleotide sequence that has at least 90%, 91%, 92%, 93%, 94%, 95%, 96%, 97%, 98%, 99%, or 100% identity to any one of SEQ ID NOs: 1123, 1128, 1133, 1136, 1141, 1146, 1151, 1156, 1161, 1166, 1171, 1218, 1223, 1253, and 1258, 1275, 1306, 1931, 1934, and 1937.

194. The system of any one of claims 190-191, wherein (i) the polypeptide comprises a sequence that has at least 90%, 91%, 92%, 93%, 94%, 95%, 96%, 97%, 98%, 99%, or 100% identity to any one of SEQ ID NO: 1175, 1177, 1180, and 1182, optionally a sequence that has at least 90%, 91%, 92%, 93%, 94%, 95%, 96%, 97%, 98%, 99%, or 100% identity to SEQ ID NO: 1177 or 1182; or (ii) the polypeptide is encoded by a nucleotide sequence that has at least 90%, 91%, 92%, 93%, 94%, 95%, 96%, 97%, 98%, 99%, or 100% identity to any one of SEQ ID NOs: 1174, 1176, 1179, and 1181, optionally a nucleotide sequence that has at least 90%, 91%, 92%, 93%, 94%, 95%, 96%, 97%, 98%, 99%, or 100% identity to SEQ ID NOs: 1176 or 1181.

195. A fusion polypeptide comprising a polK comprising GE411-412RV mutations relative to SEQ ID NO: 1021, a SpyCas9 nickase (nCas9), and a heterologous nuclear localization signal (NLS) or a nucleic acid encoding the fusion polypeptide.

196. The fusion polypeptide or nucleic acid of claim 195, wherein the polK comprises (1) an amino acid sequence that has at least 90%, 91%, 92%, 93%, 94%, 95%, 96%, 97%, 98%, or 99% identity to amino acid residues 19-526 of SEQ ID NO: 1021; or (2) an amino acid sequence that has at least 90%, 91%, 92%, 93%, 94%, 95%, 96%, 97%, 98%, 99%, or 100% identity to SEQ ID NO: 1021; or (3) an amino acid sequence of residues 19-526 of SEQ ID NO: 1021.

197. The fusion polypeptide or nucleic acid of any one of claim 195 or 196, wherein the polk is encoded by a nucleotide sequence that has at least 90%, 91%, 92%, 93%, 94%, 95%, 96%, 97%, 98%, 99%, or 100% identity to any one of SEQ ID NOs: 1020 or 1205.

198. The fusion polypeptide or nucleic acid of any one of claims 195-197, wherein the polk and nCas9 are in a sequence from N-terminus to C-terminus, (i) polk-nCas9; or (ii) nCas9-polK.

199. The fusion polypeptide or nucleic acid of any one of claims 195-198, further comprising a peptide linker or a heterologous nuclear localization signal (NLS), optionally wherein the polk and nCas9 are covalently linked sequence from N-terminus to C-terminus selected from:
1) NLS-nCas9-NLS-polK;
2) NLS-nCas9-linker-NLS-linker-polK;
3) polK-NLS-nCas9-NLS;
4) polK-linker-NLS-linker-nCas9-linker-NLS;
5) NLS-nCas9-NLS-polK (GE411-412RV);
6) NLS-nCas9-linker-NLS-polK (GE411-412RV);
7) NLS-nCas9-NLS-polK (19-526);
8) NLS-nCas9-linker-NLS-linker-polK (19-526);
9) polK (19-526)-NLS-nCas9-NLS;
10) polK (19-526)-Linker-NLS-linker-nCas9-linker-NLS;
11) polK (GE411-412RV)-NLS-NLS-nCas9;
12) polK (GE411-412RV)-linker-NLS-linker-NLS-nCas9;
13) NLS-nCas9-NLS-polK (19-526, GE411-412RV);
14) NLS-nCas9-linker-NLS-linker-polK (19-526, GE411-412RV);
15) NLS-nCas9-NLS-polK-NLS;
16) NLS-nCas9-linker-NLS-linker-polK-linker-NLS;
17) NLS-nCas9-NLS-polK (1-526, GE411-412RV)-NLS;
18) NLS-nCas9-linker-NLS-linker-polK (1-526, GE411-412RV)-linker-NLS;
19) nCas9-polK (1-526);
20) polK (1-526, GE411-412RV)-nCas9-NLS-heterodimerization domain, optionally wherein the heterodimerization domain comprises a coiled coil heterodimerization domain, optionally an EI domain or a KI domain;
21) polK (1-526, GE411-412RV)-linker-NLS-nCas9-linker-NLS-heterodimerization domain, optionally wherein the heterodimerization domain comprises a coiled coil heterodimerization domain, optionally an EI domain or KI domain;
optionally wherein the order is NLS-nCas9-linker-NLS-linker-polK (1-526, GE411-412RV)-linker-NLS, optionally further comprising a heterodimerization domain comprising a coiled coil heterodimerization domain, optionally an EI domain or KI domain.

200. The fusion polypeptide of any one of claims 195-199, wherein polk and nCas9 are covalently linked sequentially in a polypeptide from N-terminus to C-terminus, wherein the order is:
1) SV40 NLS-nCas9-linker-SV40 NLS-linker-polK (1-526, GE411-412RV)-linker-SV40NLS;
2) SV40 NLS-nCas9-linker-SV40 NLS-linker-polK (1-526, GE411-412RV)-linker-SV40 NLS-linker-EI domain; or
3) SV40 NLS-nCas9-linker-SV40 NLS-linker-polK (1-526, GE411-412RV)-linker-SV40 NLS-linker-KI domain.

201. The fusion polypeptide or nucleic acid of any one of claims 195-200, wherein (i) the polypeptide comprises a sequence selected from any one of SEQ ID NOs: 1024, 1027, 1030, 1036, 1039, 1042, 1045, 1048, 1053, 1056, 1059, 1074, 1200, 1202, 1229, 1234, and 1268, 1300, 1373, 1388, 1447, 1453, 1923, 1926, and 1929; or (ii) the polypeptide is encoded by a nucleic acid comprising a nucleotide sequence that has at least 90%, 91%, 92%, 93%, 94%, 95%, 96%, 97%, 98%, 99%, or 100% identity to any one of SEQ ID NOs: 1023, 1026, 1029, 1035, 1038, 1041, 1044, 1047, 1052, 1055, 1058, 1073, 1199, 1201, 1228, 1233, and 1267, and 1299, 1372, 1387, 1446, 1452, 1922, 1925, and 1928.

202. A fusion polypeptide comprising a T5 pol comprising D164A and E166A mutations relative to SEQ ID NO: 1116, a SpyCas9 nickase (nCas9), a heterologous nuclear localization signal (NLS), and optionally a heterodimerization domain, or a nucleic acid encoding the fusion polypeptide thereof.

203. The fusion polypeptide or nucleic acid of claim 202, wherein the T5 pol comprises an amino acid sequence that has at least 90%, 91%, 92%, 93%, 94%, 95%, 96%, 97%, 98%, 99%, or 100% identity to any one of SEQ ID NO: 1116 or 1121, optionally further wherein the T5 pol comprises a heterodimerization domain.

204. The fusion polypeptide or nucleic acid of claim 202 or 203, wherein the T5 pol comprises an N-terminal deletion of 30 amino acids relative to SEQ ID NO: 1116.

205. The fusion polypeptide or nucleic acid of any one of claims 202-204, wherein the T5 pol is encoded by a nucleotide sequence that has at least 90%, 91%, 92%, 93%, 94%, 95%, 96%, 97%, 98%, 99%, or 100% identity to any one of SEQ ID NOs: 1115 or 1120.

206. The fusion polypeptide or nucleic acid of any one of claims 202-205, wherein the T5 pol and the nCas9 are in a sequence from N-terminus to C-terminus, (i) nCas9-T5 pol or (ii) T5 pol-nCas9.

207. The fusion polypeptide or nucleic acid of any one of claims 202-206, wherein the T5Pol and nCas9 are covalently linked in sequence from N-terminus to C-terminus selected from:
1) nCas9-T5Pol-NLS
2) nCas9-linker-T5Pol-linker-NLS;
3) T5Pol-nCas9-NLS;
4) T5Pol-linker-nCas9-linker-NLS;
5) NLS-nCas9-NLS-T5Pol-binding site-NLS-NLS;
6) NLS-nCas9-linker-NLS-linker-T5Pol-linker-MCP-linker-NLS-linker-NLS;
7) nCas9-linker-T5Pol-linker-NLS;
8) nCas9-T5Pol(N30del,)-NLS;
9) nCas9-linker-T5Pol(N30del)-linker-NLS;
10) nCas9-T5Pol(A593R)-NLS;
11) nCas9-linker-T5Pol(DA593R)-linker-NLS;
12) NLS-nCas9-NLS-T5Pol-NLS-NLS;
13) NLS-nCas9-linker-NLS-linker-T5Pol-linker-NLS-linker-NLS;
14) nCas9-NLS-T5Pol;
15) nCas9-linker-NLS-linker-T5Pol;
16) NLS-nCas9-NLS-T5Pol-NLS-NLS;
17) NLS-nCas9-linker-NLS-linker-T5Pol-linker-NLS-NLS;
18) nCas9-T5Pol(K425I)-NLS;
19) nCas9-linker-T5Pol(K425I)-linker-NLS;
20) nCas9-T5Pol-NLS-heterodimerization domain, optionally wherein the heterodimerization domain comprises a coiled coil heterodimerization domain, optionally an EI domain or a KI domain; or
21) nCas9-linker-T5Pol-linker-NLS-linker-heterodimerization domain, optionally wherein the heterodimerization domain comprises a coiled coil heterodimerization domain, optionally an EI domain or a KI domain,
optionally, wherein the T5pol comprises a mutation at D164 or E166 relative to SEQ ID NO: 1116, optionally wherein the T5 pol comprises D164A and E166A mutations relative to SEQ ID NO: 1116.

208. The fusion polypeptide or nucleic acid of any one of claims 202-207, wherein the T5 pol and nCas9 are covalently linked sequentially in a polypeptide from N-terminus to C-terminus in order wherein the order is NLS-nCas9-linker-NLS-linker-T5Pol-linker-NLS-NLS; optionally, wherein the T5pol comprises a mutation at D164 or E166 relative to SEQ ID NO: 1116, optionally wherein the T5 pol comprises D164A and E166A mutations relative to SEQ ID NO: 1116.

209. The fusion polypeptide or nucleic acid of any one of claims 202-208, wherein the polypeptide comprises a sequence selected from any one of SEQ ID NO: 1124, 1127, 1129, 1132, 1134, 1137, 1140, 1142, 1145, 1147, 1150, 1152, 1155, 1157, 1160, 1162, 1165, 1167, 1170, 1172, 1216, 1219, 1224, 1254, and 1259, 1276, 1303, 1307, 1376, 1450, 1932, 1935, and 1938, optionally a sequence selected from any one of SEQ ID NO: 1124, 1129, 1134, 1137, 1142, 1147, 1152, 1157, 1162, 1167, 1172, 1219, 1224, 1254, and 1259, 1276, 1307, 1932, 1935, and 1938.

210. The fusion polypeptide or nucleic acid of any one of claims 202-209, wherein the polypeptide is encoded by a nucleotide sequence that has at least 90%, 91%, 92%, 93%, 94%, 95%, 96%, 97%, 98%, 99%, or 100% identity to any one of SEQ ID NOs: 1123, 1126, 1128, 1131, 1133, 1136, 1139, 1141, 1144, 1146, 1149, 1151, 1154, 1156, 1159, 1161, 1164, 1166, 1169, 1171, 1215, 1218, 1223, 1253, and 1258, 1275, 1302, 1306, 1375, 1449, 1931, 1934, and 1937, optionally a nucleotide sequence that has at least 90%, 91%, 92%, 93%, 94%, 95%, 96%, 97%, 98%, 99%, or 100% identity to any one of SEQ ID NOs: 1123, 1128, 1133, 1136, 1141, 1146, 1151, 1156, 1161, 1166, 1171, 1218, 1223, 1253, and 1258, 1275, 1306, 1931, 1934, and 1937.

211. A system comprising (i) a fusion polypeptide comprising a DNA-dependent DNA polymerase and a SpyCas9 nickase (nCas9), a nucleic acid encoding the fusion polypeptide, (ii) a template guide RNA (tgRNA), and (iii) an enhancer of template-based genome editing, wherein:
1) the fusion polypeptide comprising the DNA-dependent DNA polymerase and the nCas9 is a fusion polypeptide of any one of claim 45-65, 82-111, or 582-627;
2) the tgRNA comprises a first polynucleotide and a second polynucleotide, wherein:
i) the first polynucleotide comprises a SpyCas9 guide RNA comprising, from 5′ to 3′:
1) a spacer that is complementary to a target region of a target strand of a DNA duplex target nucleic acid; and
2) a scaffold; and
ii) the second polynucleotide is operably linked to the first polynucleotide and comprises, from 5′ to 3′:
1) a template sequence that is at least 10 nucleotides in length and complementary to at least a portion of a non-target strand of the DNA duplex target nucleic acid, wherein the template sequence comprises a 3′ end and a 5′ end; and wherein the template comprises DNA nucleotides;
2) a DNA-dependent DNA polymerase recruiting sequence (DRS) that is 6-20 nucleotides in length, optionally 8-17 or 10-14 nucleotides in length and having a 3′ end and a 5′ end; wherein the 5′ end of the DRS is covalently attached to the 3′ end of the template sequence, and wherein the DRS comprises a sequence that is complementary to at least 6 consecutive nucleotides of a DRS-complementary region of the non-target strand of the DNA duplex target nucleic acid; and
3) the enhancer of template-based genome editing.

212. The system of claim 211, wherein the enhancer of template-based genome editing is an inhibitor of deoxynucleotide triphosphohydrolase, optionally wherein the inhibitor of deoxynucleotide triphosphohydrolase comprises a Vpx, a BGLF4, an M97, or an ORF36.

213. The system of claim 211 or 212, wherein the enhancer of template-based genome editing is selected from:
i) a Vpx protein comprising an amino acid sequence that has at least 90%, 91%, 92%, 93%, 94%, 95%, 96%, 97%, 98%, 99%, or 100% identity to any one of SEQ ID NOs: 1464, 1469, and 1503-1505, optionally wherein the Vpx protein includes or lacks an N-terminal methionine relative to any one of SEQ ID NO: 1464, 1469, and 1503-1505, or a nucleic acid encoding the amino acid sequence;
ii) a BGLF4 protein comprising an amino acid sequence that has at least 90%, 91%, 92%, 93%, 94%, 95%, 96%, 97%, 98%, 99%, or 100% identity to SEQ ID NO: 1517 or 1518, optionally wherein the BGLF4 protein includes or lacks an N-terminal methionine relative to SEQ ID NO: 1517 or 1518, or a nucleic acid encoding the amino acid sequence; or
iii) an M97 protein comprising an amino acid sequence that has at least 90%, 91%, 92%, 93%, 94%, 95%, 96%, 97%, 98%, 99%, or 100% identity to SEQ ID NO: 1500, optionally wherein the M97 protein includes or lacks an N-terminal methionine relative to SEQ ID NO: 1500, or a nucleic acid encoding the amino acid sequence; or
iv) a KSHV ORF36 protein comprising an amino acid sequence that has at least 90%, 91%, 92%, 93%, 94%, 95%, 96%, 97%, 98%, 99%, or 100% identity to SEQ ID NO: 1576, optionally wherein the KSHV ORF36 protein includes or lacks an N-terminal methionine relative to SEQ ID NO: 1576, or a nucleic acid encoding the amino acid sequence.

214. The system of any one of claims 211-213, wherein the inhibitor of deoxynucleotide triphosphohydrolase is encoded by a nucleotide sequence that has at least 90%, 91%, 92%, 93%, 94%, 95%, 96%, 97%, 98%, 99%, or 100% identity to any one of SEQ ID NOs: 1462, 1463, 1468, 1470, 1499, 1501, 1502, 1521-1523, 1574-1575, and 1585, and 3530.

215. The system of claim 211, wherein the enhancer of template-based genome editing is a DNA repair protein, or a nucleic acid encoding the amino acid sequence.

216. The system of claim 215, wherein the DNA repair protein is a single-stranded endonuclease that removes a 5′ flap at the 3′ end of a nick, optionally a flap structure-specific endonuclease 1 (FEN1) or MLH1.

217. The system of any one of claims 215-216, wherein the DNA repair protein is selected from:
i) a FEN1 protein comprising an amino acid sequence that has at least 90%, 91%, 92%, 93%, 94%, 95%, 96%, 97%, 98%, 99%, or 100% identity to any one of SEQ ID NOs: 1467, 1475, 1480, 1490, 1495, 1510-1516 and 1550-1555, optionally wherein the FEN1 protein includes or lacks an N-terminal methionine relative to any one of SEQ ID NOs: 1467, 1475, 1485, 1490, 1495, and 1510-1516, or a nucleic acid encoding the amino acid sequence; or
ii) a MLH1 protein comprising an amino acid sequence that has at least 90%, 91%, 92%, 93%, 94%, 95%, 96%, 97%, 98%, 99%, or 100% identity to any one of SEQ ID NOs: 1572 or 1573, optionally wherein the MLH1 protein includes or lacks an N-terminal methionine relative to any one of SEQ ID NOs: 1572 or 1573, or a nucleic acid encoding the amino acid sequence.

218. The system of any one of claims 211-217, comprising at least two enhancers of template-based genome editing,

219. The system of claim 218, wherein the at least two enhancers of template-based genome editing comprise Vpx and FEN-1.

220. The system of claim 219, wherein the at least two enhancers comprising Vpx and FEN1 are encoded on a same polypeptide, optionally wherein the polypeptide encoding Vpx and FEN1 comprises an amino acid sequence that has at least 90%, 91%, 92%, 93%, 94%, 95%, 96%, 97%, 98%, 99%, or 100% identity to any one of SEQ ID NOs: 1578, 1580, 1582, and 1584; or wherein the polypeptide encoding Vpx and FEN1 is encoded by a nucleic acid sequence that has at least 90%, 91%, 92%, 93%, 94%, 95%, 96%, 97%, 98%, 99%, or 100% identity to any one of SEQ ID NOs: 1577, 1579, 1581, and 1583, 3523, 3524, 3526, and 3527.

221. The system of any one of claim 1-59, 139-194, or 211-220 or the fusion protein or nucleic acid of any one of claims 195-210, wherein the heterologous NLS is independently selected from Nucleoplasmin NLS, C-Myc NLS, and SV40 NLS and the linker is independently selected from SEQ ID NOs: 1596, 1597, 1600, 1601, 1605, 1661, 1735, 1738, and 1815.

222. The system of any one of claim 1-59, 139-194, or 211-220 or the fusion protein or nucleic acid of any one of claims 195-210, wherein the linker comprises an amino acid sequence of any one of SEQ ID NOs: 1596-1665, 1735, 1738, 1815, 1825, 1827, and 1829-1830.

223. The nucleic acid of any one of claims 195-210 and 221-222, wherein the nucleotide sequence encoding a polypeptide comprising the nCas9 or DNA-dependent DNA polymerase, or the fusion protein comprises:
i) a Cap selected from Cap0 or Cap1;
ii) a 5′ UTR selected from SEQ ID NOs: 1720-1723;
iii) a Kozak sequence selected from SEQ ID NOs: 1724 and 1725;
iv) a 3′ UTR selected from SEQ ID NOs: 1684 and 1740;
v) a poly-A tail selected from SEQ ID NOs: 1741 and 1742; or
vi) a combination of two, three, four, or five of (i)-(v).

224. The system of any one of claims 1-59, 139-194, and 211-223, wherein the DNA-dependent DNA polymerase, nCas9, and enhancers are provided as one or more polypeptides.

225. The system of any one of claims 1-59, 139-194, and 211-223, wherein the DNA-dependent DNA polymerase, nCas9, and enhancers are provided as one or more nucleic acids.

226. A system comprising the nucleic acid of any one of claims 195-210 or a composition comprising the nucleic acid of any one of claims 195-210 and an enhancer of template-based genome editing, optionally further comprising a template guide RNA of any one of claims 60-138.

227. The system or composition of claim 226, wherein the enhancer of template-based genome editing comprises a nucleic acid encoding the enhancer of template-based genome editing or an enhancer of template-based genome editing polypeptide.

228. The system or composition of claim 226 or 227, wherein the enhancer of template-based genome editing is an inhibitor of deoxynucleotide triphosphohydrolase.

229. The system of any one of claims 226-228, wherein the deoxynucleotide triphosphohydrolase comprises SAM domain and HD domain-containing protein 1 (SAMHD1).

230. The system or composition of claim 229, wherein the inhibitor of SAMHD1 comprises microRNA, shRNA, or siRNA that hybridizes to a SAMHD1 mRNA.

231. The system or composition of claim 229, wherein the inhibitor of SAMHD1 comprises a Vpx, a BGLF4, an M97, or KSHV ORF36.

232. The system or composition of claim 227 or 228, wherein the enhancer of template-based genome editing is a DNA repair protein, or a nucleic acid encoding the amino acid sequence.

233. The system or composition of claim 232, wherein the DNA repair protein is flap structure-specific endonuclease 1 (FEN1) or wherein the DNA repair protein is MLH1.

234. The system or composition of any one of claims 232-233, wherein the DNA repair protein comprises a heterodimerization domain or further comprises an additional heterodimerization domain.

235. The system or composition of any one of claim 232 or 234, wherein the DNA repair protein is selected from:
i) a FEN1 protein comprising an amino acid sequence that has at least 90%, 91%, 92%, 93%, 94%, 95%, 96%, 97%, 98%, 99%, or 100% identity to any one of SEQ ID NOs: 1467, 1475, 1485, 1490, 1495, and 1510-1516, optionally wherein the FEN1 protein includes or lacks an N-terminal methionine relative to any one of SEQ ID NOs: 1467, 1475, 1485, 1490, 1495, and 1510-1516, or a nucleic acid encoding the amino acid sequence; or
ii) MLH1 protein comprising an amino acid sequence that has at least 90%, 91%, 92%, 93%, 94%, 95%, 96%, 97%, 98%, 99%, or 100% identity to any one of SEQ ID NOs: 1572 or 1573, optionally wherein the MLH1 protein includes or lacks an N-terminal methionine relative to any one of SEQ ID NOs: 1572 or 1573, or a nucleic acid encoding the amino acid sequence.

236. The system or composition of any one of claims 226-235 comprising two nucleic acids encoding two enhancers of template-based genome editing, optionally comprising (i) Vpx and FEN-1 or (ii) M97 and FEN-1.

237. The system or composition of any one of claims 1-59, 139-194, and 221-236, wherein further comprises a nicking guide RNA (ngRNA).

238. The system or composition of claim 237, wherein the ngRNA comprises a spacer that is complementary to the non-target strand of the DNA duplex target nucleic acid.

239. The system or composition of claim 238, wherein the ngRNA comprises a spacer that hybridizes to a sequence on the non-target strand such that (i) the nick site of the ngRNA is within 200 nucleotides 5′ or 3′ from the nick site of the template guide RNA; (ii) the nick site of the ngRNA is 20-200 nucleotides 5′ or 3′ from the nick site of the template guide RNA; or (iii) the nick site of the ngRNA is outside of the genomic locus of the spacer sequence of the template guide RNA.

240. The system or composition of any one of claims 237-239, wherein the ngRNA comprises, from 5′ to 3′, a guide sequence and any one of SEQ ID NOs: 5, 27, 35, 210, 223, 269, 370, 201-208, 373-374, and 393-400; (ii) a SpyCas9 guide RNA comprising, from 5′ to 3′, a guide sequence and a nucleotide sequence that is at least 90%, 91%, 92%, 93%, 93%, 94%, 95%, 96%, 97%, 98%, 99%, or 100% of any one of SEQ ID NOs: 5, 27, 35, 210, 223, 269, 370, 201-208, 373-374, 393-400, 429, and 430; or (iii) a SpyCas9 guide RNA comprising a nucleotide sequence that is at least 90%, 91%, 92%, 93%, 93%, 94%, 95%, 96%, 97%, 98%, 99%, or 100% of any one of SEQ ID NOs: 310, 319-326, 347-349, 371, 384-392, 434, 438

241. The system, fusion protein, or composition of any one of claims 1-59 and 139-240, (i) wherein the nCas9 comprises an amino acid sequence that has at least 90%, 91%, 92%, 93%, 94%, 95%, 96%, 97%, 98%, 99%, or 100% identity to any one of SEQ ID NOs: 972, 974, 976, 982, 984, 986, 988, 990, 992, 994, 996, 998, 1000, and 1014, optionally wherein the nCas9 comprises an amino acid sequence of any one of SEQ ID NOs: 972, 974, 976, 982, 984, 986, 988, 990, 992, 994, 996, 998, 1000, and 1014, optionally wherein the nCas9 protein includes or lacks an N-terminal methionine relative to any one of SEQ ID NOs: 972, 974, 976, 982, 984, 986, 988, 990, 992, 994, 996, 998, 1000, and 1014; or (ii) wherein the nCas9 is encoded by a nucleotide sequence that has at least 90%, 91%, 92%, 93%, 94%, 95%, 96%, 97%, 98%, 99%, or 100% identity to any one of SEQ ID NOs: 971, 973, 975, 981, 983, 985, 987, 989, 991, 993, 995, 997, 999, and 1013, optionally wherein the nCas9 is encoded by a nucleotide sequence of any one of SEQ ID NOs: 971, 973, 975, 981, 983, 985, 987, 989, 991, 993, 995, 997, 999, and 1013, optionally wherein the nCas9 protein includes or lacks a start codon or stop codon relative to any one of SEQ ID NOs: 971, 973, 975, 981, 983, 985, 987, 989, 991, 993, 995, 997, 999, and 1013.

242. The system, fusion protein, or composition of any one of claims 1-59 and 139-241, wherein the nCas9 comprises an H840A point mutation.

243. The system, fusion protein, or composition of any one of claims 226-242, wherein the nucleotide sequence encoding the nCas9, DNA-dependent DNA polymerase, nCas9/DNA-dependent DNA polymerase fusion protein, enhancer of template-based genome editing, or Sso7 DNA binding accessory factor comprises a 5′ cap selected from Cap0, Cap1, and Cap2.

244. The system, fusion protein, or composition of any one of claims 226-243, wherein the polynucleotide further comprises a poly-adenylated (poly-A) tail sequence or a polyadenylation signal sequence.

245. The system, fusion protein, or composition of any claim 243 or 244, wherein the poly-A sequence comprises a sequence of any one of SEQ ID NOs: 1692-1693 and 1741-1742.

246. The system, fusion protein, or composition of any one of claims 226-245, wherein at least 70%, 75%, 80%, 85%, 90%, or 95% of the uridine in the nucleic acid is substituted with a modified uridine, optionally wherein the modified uridine is one or more of N1-methyl-pseudouridine, pseudouridine, 5-methoxyuridine, or 5-iodouridine.

247. The system or composition of any one of claims 1-59, 139-194, and 211-246, further comprising a template guide RNA, if not present, wherein the template guide RNA is a template guide RNA of any one of claims 60-138.

248. The system or composition of claim 247, wherein the DRS bound to the DRS-complementary region in the non-target strand of the DNA duplex target nucleic acid forms a binding site for a DNA-dependent DNA polymerase, optionally wherein the DNA-dependent DNA polymerase is polK or T5 pol.

249. The system or composition of any one of claims 247-248, wherein (i) the DRS has 0, 1, 2, 3, or 4 mismatches to the corresponding genomic sequence; (ii) 6 terminal nucleotides at the 5′ end of the DRS have 0, 1, 2, or 3 mismatches to the corresponding genomic sequence; (iii) the first 3 nucleotides at the 3′ end of the DRS have 0, 1, or 2 mismatches to the genomic sequence; (iv) the DRS is 100% complementary to the corresponding genomic sequence for no more than 6 consecutive nucleotides, optionally no more than 4 consecutive nucleotides, without a mismatch interruption; or (v) 10 terminal nucleotides at the 5′ end of the DRS are 100% complementary to the corresponding genomic sequence.

250. The system or composition of any one of claims 247-249, wherein the at least one mismatched RNA nucleotides in the DRS comprises a modified RNA nucleotide, optionally all of the mismatched RNA nucleotides in the DRS comprise a modified RNA nucleotide.

251. The system or composition of any one of claims 247-250, wherein the template sequence is 10-10000 nucleotides in length, optionally 10-20, 10-100, 10-500.

252. The system or composition of any one of claims 247-251, wherein the template sequence is 14-18 nucleotides in length, optionally 14 or 15 nucleotides in length.

253. The system or composition of any one of claims 247-252, wherein the template sequence further comprises at least 2 nucleotides 5′ of a sequence to be edited by template-based genome editing, wherein the at least 8 nucleotides across the template base pair with the non-target strand of the DNA duplex target nucleic acid.

254. The system or composition of any one of claims 247-253, wherein the template sequence further comprises at least 5 nucleotides 5′ of a sequence to be edited by template-based genome editing, wherein the at least 10 nucleotides across the template base pair with the non-target strand of the DNA duplex target nucleic acid.

255. The system or composition of any one of claims 247-254, wherein the template sequence comprises a sequence of at least 5 consecutive nucleotides that base pair with the non-target strand of the DNA duplex target nucleic acid.

256. The system or composition of any one of claims 247-255, wherein the template sequence comprises a mismatch to the genomic sequence at at least one of positions 1-6, optionally at at least one of positions 5-6 5′ of the template junction with the DRS (positions T1-T6, optionally positions T5-T6).

257. The system or composition of claim 256, wherein the template sequence comprises a mismatch to the genomic sequence at at least one of positions 1-6, optionally at at least one of positions 5-6 5′ of the template junction with the DRS introduces a silent mutation.

258. The system or composition of claim 256, wherein the template sequence comprises a mismatch to the genomic sequence at at least one of positions T1-T6, optionally at at least one of positions T5-T6 5′ of the template junction with the DRS, wherein the template introduces a change in the amino acid sequence encoded by the genomic sequence.

259. The system or composition of any one of claims 247-258, wherein the template sequence comprises an insertion sequence relative to the corresponding genomic sequence.

260. The system or composition of claim 259, wherein the template sequence comprises an insertion sequence of up to 500, 1000, 3500, or 5000 nucleotides in length, optionally up to 10,000 nucleotides in length.

261. The system or composition of claim 259 or 260, wherein the length of the templated insertion is 1-10, 1-20, 1-50, 1-75, or 1-100 nucleotides in length.

262. The system or composition of any one of claims 247-261, wherein the template sequence comprises a sequence for directing a deletion relative to the corresponding genomic sequence.

263. The system or composition of claim 262, wherein the template sequence comprises a sequence to direct a deletion of at least 3, 10, 15, 20, 30, 40, or 50 nucleotides in length, optionally wherein the template sequence comprises a sequence to direct a deletion of up to 3500 nucleotides in length, optionally up to 10,000 nucleotides in length.

264. The system or composition of any one of claims 247-263, wherein the template sequence comprises at least one mismatch, optionally at least 3 mismatches relative to the corresponding genomic sequence wherein the mismatch provides the template sequence for template-based genome editing comprising a substitution.

265. The system or composition of any one of claims 247-264, wherein the template sequence comprises 1-10 mismatches relative to the corresponding genomic sequence wherein the mismatch provides the template sequence for template-based genome editing comprising a substitution.

266. The system or composition of claim 264 or 265, wherein the substitution is a transition or transversion.

267. The system or composition of any one of claims 264-266, wherein the template-based edit in the genomic sequence results in a change in an amino acid sequence encoded by a locus in which the genomic sequence is present.

268. The system or composition of any one of claims 264-267, wherein the template-based edit in the genomic sequence does not result in a change in an amino acid sequence encoded by a locus in which the genomic sequence is present.

269. The system or composition of any one of claims 264-268, wherein the template further comprises an insertion sequence or a sequence for directing a deletion.

270. The system, tgRNA, or composition of any one of claims 1-194 and 211-269, wherein the SpyCas9 guide RNA comprises, from 5′ to 3′, a spacer sequence and a SpyCas9 guide RNA scaffold sequence.

271. The system, tgRNA, or composition of any one of claims 1-194 and 211-270, wherein the SpyCas9 guide RNA scaffold sequence comprises an internal linker.

272. The system, tgRNA, or composition of any one of claims 1-194 and 211-271, wherein the spacer of the guide RNA comprises a DNA nucleotide.

273. The system, tgRNA, or composition of any one of claims 1-194 and 211-272, wherein the spacer or the scaffold of the guide RNA comprises a modified nucleotide selected from a 2′-O-methyl (2′-O-Me) modified nucleotide, or a 2′-F modified nucleotide or wherein the spacer or the scaffold of the guide RNA comprises a phosphorothioate (PS) bond between nucleotides.

274. The system, tgRNA, or composition of any one of claims 1-194 and 211-273, wherein the guide RNA comprises a DNA nucleotide at at least 2, 3, 4, 5, 6, 7, 8, 9, or 10 nucleotides of the spacer, optionally wherein the guide RNA comprises a DNA nucleotide at one or more of positions S11, S12, and S18.

275. The system, tgRNA, or composition of any one of claims 1-194 and 211-274,

276. The system, tgRNA, or composition of any one of claims 1-194 and 211-275, wherein the guide RNA comprises a 2′-O-Me or 2′-F modified nucleotide at at least 2, 3, 4, 5, 6, 7, 8, 9, or 10 nucleotides of the spacer, optionally wherein the guide RNA comprises 2′-O-Me or 2′-F modified nucleotides at one or more of positions S4, S5, S9, S10, S13, S14, S17 and S20.

277. The system, tgRNA, or composition of any one of claims 1-194 and 211-276, wherein the guide RNA terminates with 1-4 uridine nucleotides or 1-4 modified uridine nucleotides, optionally a modified uridine nucleotide at the 3′ end.

278. The system, tgRNA, or composition of any one of claims 1-194 and 211-277, wherein the guide RNA comprises a 3′ end modification and 5′ end modification.

279. The system, tgRNA, or composition of claim 278, wherein the 3′ end modification or 5′ end modification comprises or further comprises a 2′-O-methyl (2′-O-Me) modified nucleotide or a phosphorothioate (PS) linkage between nucleotides.

280. The system, tgRNA, or composition of any one of claims 1-194 and 211-279, wherein the SpyCas9 guide RNA comprises, from 5′ to 3′, a spacer sequence and a scaffold sequence having at least 90%, 91%, 92%, 93%, 94%, 95%, 96%, 97%, 98%, 99%, or 100% identity to any one of SEQ ID NOs: 4, 5, 27, 370, 429, and 430, or the scaffold portion of any one of the sequences listed in Tables 1A-1D.

281. The system, tgRNA, or composition of any one of claims 1-194 and 211-280, wherein the SpyCas9 guide RNA comprises a modified nucleotide sequence having at least 90% identity to any one of (a) SEQ ID NOs: 201-218, 223, 224, 225-231, 232-235, 236, 237-239, 240-269, 270-276, 300-318, 319, 323-325, 326-344, 345-349, and 372-374, 375-383, and 431-434, or any one of the sequences listed in Tables 1C and 1D, or a modified nucleotide sequence having at least 90%, 95%, 98% identity to any one of SEQ ID NOs: 350-374 and 384-400, 429-430, 435-454, or any one of the sequences listed in Table 4; or (b) SEQ ID NOs: 201-218, 223, 224, 225-269, 270-271, 272-273, 274, 275 276, 300-318, 319, 323-325, 326-344, 345-346, 347-349, 372-383, and 431-433, 434 or any one of the sequences listed in Tables 1C and 1D, or a modified nucleotide sequence having at least 90%, 95%, 98% identity to any one of SEQ ID NOs: 350-374, 384-400, and 429-430, and 435-454 or any one of the sequences listed in Table 4; optionally wherein the SpyCas9 guide RNA comprises a modified nucleotide sequence of any one of SEQ ID NOs: 201-218, 223, 224, 225-250, 269, 272-273, 274, 275, 276, 301-318, 319, 323-325, 326-334, 347-349, 372-374, and 431-433, 434; or wherein the SpyCas9 guide RNA comprises a modified nucleotide sequence of any one of SEQ ID NOs: 201-218, 224, 225-231, 232-235, 236, 237-239, 240-269, 270-273, 274, 275, 276, 300-318, 319, 323-325, 326-344, 345-349, 372-374, and 375-383 or any one of the sequences listed in Tables 1C and 1D or a modified nucleotide sequence of any one of SEQ ID NOs: 350-374 and 384-400, 429-430, 435-454 or any one of the sequences listed in Table 4; or (b) SEQ ID NOs: 201-218, 224, 225-269, 270-271, 272-273, 274, 275, 276, 300-318, 319, 323-325, 326-344, 345-346, 347-349, 372-383, and 431-433, 434 or any one of the sequences listed in Tables 1C and 1D or a modified nucleotide sequence of any one of SEQ ID NOs: 350-374 and 384-400, and 435-454 or any one of the sequences listed in Table 4;
optionally wherein the SpyCas9 guide RNA comprises a modified nucleotide sequence of any one of SEQ ID NOs: 201-218, 224, 225-250, 269, 272-273, 274, 275, 276, 301-318, 319, 323-325, 326-334, 347-349, 372-374, and 431-433, 434.

282. The system, tgRNA, or composition of any one of claims 1-194 and 211-281, wherein the SpyCas9 guide RNA comprises:
i) mN*mN*mN*NNNNNNNNNNNNNNNNNGUUUUAGA(L3)AAGUUAAAAUAAGGCU AGUCCGUUAUCAC(L1)GGGCACCGAGUCGGmU*mG*mC*mU (SEQ ID NO: 371) or a sequence having at least 90%, 95%, 98%, or 99% identity to SEQ ID NO: 371;
ii) mN*mN*mN*NNNNNNNNNNNNNNNNNmGUUUUAGmAmGmC(L1)mGmCmAAGUU mAAmAAmUmAmAGmGCUmAGUCmCmGUUAUmCAmC(L1)mGGmGmCmAmCmCm GmAGUCmGmGmUmGmC (SEQ ID NO: 347) or a sequence having at least 90%, 95%, 98%, or 99% identity to SEQ ID NO: 347; or
iii) mN*mN*mN*NNNNNNNNNNNNNNNNNGUUUUAGAmGmCmUmA(L1)mUmAmGmC AAGUUAAAAUAAGGCUAGUCCGUUAUCAC(L1)GGGCACCGAGUCGGmUmGmC (SEQ ID NO: 348) or a sequence having at least 90%, 95%, 98%, or 99% identity to SEQ ID NO: 348;
wherein * denotes that the nucleotide is linked to the next nucleotide with a PS bond, a lower case “m” denotes that the nucleotide is modified with 2′-O-Me, where N is any nucleotide.

283. A lipid nanoparticle (LNP) composition comprising the system, tgRNA, guide RNA, or composition of any one of claims 1-194 and 211-282.

284. The system, tgRNA, guide RNA, composition, or LNP composition of any one of claims 1-194 and 211-283, wherein one or more of the tgRNA, first polynucleotide, second polynucleotide, nucleic acid encoding the DNA-dependent DNA polymerase, nucleic acid encoding the nCas9, or nucleic acid encoding the fusion protein is associated with one or more LNP.

285. The system, tgRNA, guide RNA, composition, or LNP composition of any one of claims 1-194 and 211-284, wherein the DNA-dependent DNA polymerase and nCas9 are a fusion protein encoded by a single mRNA associated with one LNP; or, wherein the tgRNA is associated with a separate LNP from the LNP associated with the mRNA encoding the DNA-dependent DNA polymerase and nCas9 are a fusion protein, optionally wherein the system or composition further comprises an mRNA encoding an enhancer of template-based genome editing associated with an LNP.

286. The system, tgRNA, guide RNA, composition, or LNP composition of any one of claims 283-285, wherein the LNP comprises (i) an ionizable lipid; (ii) a helper lipid; (iii) a stealth lipid; (iv) a neutral lipid; or combinations of one or more of (i)-(iv).

287. The system, tgRNA, guide RNA, composition, or LNP composition of claim 286, wherein the ionizable lipid is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate.

288. The system, tgRNA, guide RNA, composition, or LNP composition of claim 286 or 287, wherein the helper lipid is cholesterol; the stealth lipid is PEG-DMG; or the neutral lipid is DSPC.

289. A pharmaceutical composition comprising the system, tgRNA, guide RNA, composition, fusion polypeptide, or LNP composition of any one of claims 1-194 and 211-288 and a pharmaceutically acceptable carrier.

290. A method for template-based genomic editing, comprising delivering to a cell or subject the system, tgRNA, guide RNA, composition, or LNP composition of any one of claims 1-194 and 211-288 or the fusion polypeptide or nucleic acid of any one of claims 195-210.

291. Use of the system, tgRNA, guide RNA, composition, or LNP composition of any one of claims 1-194 and 211-288 or the fusion polypeptide or nucleic acid of any one of claims 195-210 for template-based genome editing in a cell or subject.

292. The method or use of claim 290 or 291, wherein template-based genome editing results in edits comprising an insertion or deletion in a nucleotide sequence.

293. The method or use of claim 290 or 291, wherein template-based genome editing results in edits comprising a substitution in a nucleotide sequence, optionally wherein the substitution is a transition or transversion.

294. The method or use of any one of claims 290-293, wherein template-based genome editing results in at least 20%, 25%, 30%, 35%, 40%, 45%, optionally at least 50%, 55%, 60%, 65%, optionally at least 70%, 75%, or 80% templated edits as a percent of total edits in the target nucleic acid, further optionally wherein no more than 30% of the templated edits, optionally no more than 20% or 10% of the templated edits further comprise a byproduct edit.

295. The method or use of any one of claims 290-294, wherein the cell is a quiescent cell, optionally wherein the quiescent cell is selected from a hepatocyte, unactivated T-cell, muscle cell, neuronal cell, and a lung cell, and wherein at least 5% of the cells in the cell population comprise a template-based genome edit in the target nucleic acid.

296. The method or use of any one of claims 290-295, wherein the cell is a primary cell.

297. The method or use of any one of claims 290-296, wherein the cell is an immune cell and wherein at least 50% of the cells in the cell population comprise a template-based genome edit in the target nucleic acid, optionally wherein no more than 30% of the templated edits, optionally no more than 20% or 10% of the templated edits further comprise a byproduct edit.

298. The method or use of any one of claims 290-297, wherein (i) the length of the templated insertion is at least 5, 10, 15, 20, 25, 30, 35, or 40 nucleotides in length, optionally wherein the length of the templated insertion is 1-10, 1-20, 1-50, 1-75, or 1-100 nucleotides in length; or (ii) the length of the templated insertion is up to 500, 1000, 3500, 5000, or 10,000 nucleotides in length.

299. A method of producing a ligated template guide RNA (tgRNA), the method comprising:
providing a first polynucleotide comprising a 3′ end comprising at least 10 nucleotides; providing a second polynucleotide comprising a 5′ end comprising at least 10 nucleotides;
providing a splint oligonucleotide capable of annealing to the at least 10 nucleotides at the 3′ end of the first polynucleotide and the at least 10 nucleotides at the 5′ end of the second polynucleotide; and
providing a ligase to ligate the 3′ end of the first polynucleotide to the 5′ end of the second polynucleotide thereby forming a ligated tgRNA;
wherein the ligated tgRNA comprises a SpyCas9 guide RNA, a template sequence, and a DNA-dependent DNA polymerase recruiting sequence (DRS).

300. A method of producing a ligated template guide RNA (tgRNA), the method comprising:
providing a first polynucleotide comprising a 3′ end comprising at least 10 nucleotides;
providing a second polynucleotide comprising a 5′ end comprising at least 10 nucleotides and a 3′ end comprising at least 10 nucleotides;
providing a third polynucleotide comprising a 5′ end comprising at least 10 nucleotides.
providing a first splint oligonucleotide capable of annealing to the at least 10 nucleotides at the 3′ end of the first polynucleotide and the at least 10 nucleotides at the 5′ end of the second polynucleotide;
providing a second splint oligonucleotide capable of annealing to the at least 10 nucleotides at the 3′ end of the second polynucleotide and the at least 10 nucleotides at the 5′ end of the third polynucleotide; and
providing one or more ligases to ligate (i) the 3′ end of the first polynucleotide to the 5′ end of the second polynucleotide and (ii) the 3′ end of the second polynucleotide to the 5′ end of the third polynucleotide thereby forming a ligated tgRNA;
wherein the ligated tgRNA comprises a SpyCas9 guide RNA, a template sequence, and a DNA-dependent DNA polymerase recruiting sequence (DRS).

301. The method of any one of claims 299-300, wherein the ligated tgRNA is at least 100 nucleotides in length, at least 110 nucleotides in length, at least 120 nucleotides in length, at least 130 nucleotides in length, at least 140 nucleotides in length, at least 150 nucleotides in length, at least 160 nucleotides in length, or at least 170 nucleotides in length.

302. The method of any one of claims 299-301, further comprising separating the ligated tgRNA from the splint oligonucleotide.

303. The method of any one of claims 299-302, further comprising subjecting the ligated tgRNA to a purification step.

304. The method of any one of claims 299 and 301-303, wherein the splint oligonucleotide is a DNA splint oligonucleotide.

305. The method of any one of claims 299-and 301-304, wherein the splint oligonucleotide is at least 15 or at least-20 nucleotides in length, or optionally about 15-20 nucleotides in length.

306. The method of any one of claims 299-305, wherein ligation occurs at a ligation site within the scaffold of the SpyCas9 guide RNA.

307. The method of any one of claims 300-303 and 306, wherein the first and second splint oligonucleotides are DNA splint oligonucleotides.

308. The method of any one of claims 300-303 and 306-307, wherein the first splint oligonucleotide is at least at least 15, 20, 30, 40, 50, 60, 70, 80, or 90 1nucleotides in length, optionally from about 15 to 25 nucleotides in length, or the second splint oligonucleotide is at least 15, 20, 30, 40, 50, 60, 70, 80, or 90 nucleotides in length, optionally from about 15 to 25 nucleotides in length.

309. The method of any one of claims 299-308, wherein the one or more ligases comprises an RNA ligase or a DNA ligase, optionally wherein the ligase comprises a T4 DNA ligase or a T4 RNA ligase.

310. The method of any one of claims 300-303 and 306-309, wherein a first ligation occurs at a first ligation site that is flanked by RNA nucleotides and a second ligation occurs at a second ligation site that is flanked by DNA nucleotides.
